(19) United States
(12) Patent Application Publication
(10) Pub. No.: US 2026/0297042 A1
(43) Pub. Date: Oct. 1, 2026

(54) BIARYLAMIDE DERIVATIVES AND THEIR USE AS PKMYT1 INHIBITORS

(71) Applicants: CANCER RESEARCH TECHNOLOGY LIMITED, London (GB); MY-T BIO LIMITED, London (GB)

(72) Inventors: Ali RAOOF, Macclesfield (GB); Efthymios SPYRIDON-GAVRIlL, London (GB); Dan NICULESCU-DUVAZ, Macclesfield (GB); Michael BROWN, Macclesfield (GB); Deborah SMITHEN, Macclesfield (GB); Christopher FITZPATRICK, Macclesfield (GB); Oleksandr GRYTSAI, Macclesfield (GB); Mohammed ALJARAH, Macclesfield (GB); Monika LISAUSKAITE, Macclesfield (GB); Ryan BURGIN, Macclesfield (GB); Christopher Stephen KERSHAW, Macclesfield (GB); Leo LEUNG, Macclesfield (GB); Iain HAGAN, Manchester (GB); Richard MARAIS, Macclesfield (GB); Caroline SPRINGER, Macclesfield (GB)

(21) Appl. No.: 19/162,777

(22) PCT Filed: Mar. 8, 2024
(86) PCT No.: PCT/EP2024/056305

§ 371 Date: Sep. 5, 2025

(30) Foreign Application Priority Data
Mar. 9, 2023 (EP) 23386018.8

(51) Int. Cl. C07D 277/56; A61K 31/426; A61K 31/427; A61K 31/429; A61K 31/433; A61K 31/437; A61K 31/438; A61K 31/439; A61K 31/454; A61K 31/4545; A61K 31/4709; A61K 31/4725; A61K 31/496; A61K 31/506; A61K 31/519; A61K 31/53; A61K 31/5377; A61K 31/551; A61P 35/00; C07D 277/82; C07D 417/12; C07D 417/14; C07D 471/04; C07D 471/10; C07D 495/04; C07D 513/04

(52) U.S. Cl. CPC C07D 277/56; A61K 31/426; A61K 31/427; A61K 31/429; A61K 31/433; A61K 31/437; A61K 31/438; A61K 31/439; A61K 31/454; A61K 31/4545; A61K 31/4709; A61K 31/4725; A61K 31/496; A61K 31/506; A61K 31/519; A61K 31/53; A61K 31/5377; A61K 31/551; A61P 35/00; C07D 277/82; C07D 417/12; C07D 417/14; C07D 471/04; C07D 471/10; C07D 495/04; C07D 513/04

(57) ABSTRACT

The present invention pertains generally to the field of therapeutic compounds. More specifically the present invention pertains to certain biarylamide compounds of the following formula (also referred to herein as “BAA compounds”) which inhibit Protein Kinase, Membrane Associated Tyrosine/Threonine 1 (PKMYT1). The present invention also pertains to pharmaceutical compositions comprising such compounds, and the use of such compounds and compositions, both in vitro and in vivo, to inhibit PKMYT1 kinase; to treat disorders (e.g., diseases) that are ameliorated by the inhibition of PKMYT1 kinase; to treat a proliferative disorder, cancer, etc.

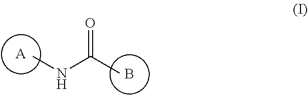

### TECHNICAL FIELD

The present invention pertains generally to the field of therapeutic compounds. More specifically the present invention pertains to certain biarylamide compounds (also referred to herein as “BAA compounds”) which inhibit Protein Kinase, Membrane Associated Tyrosine/Threonine 1 (PKMYT1). The present invention also pertains to pharmaceutical compositions comprising such compounds, and the use of such compounds and compositions, both in vitro and in vivo, to inhibit PKMYT1 kinase; to treat disorders (e.g., diseases) that are ameliorated by the inhibition of PKMYT1 kinase; to treat a proliferative disorder, cancer, etc.

### BACKGROUND

Publications are cited herein in order to more fully describe the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise.

Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art.

Protein kinase, membrane-associated tyrosine/threonine 1 (PKMYT1) A key hallmark of cancer is that cancer cells override the cell cycle controls that prevent commitment to division until the appropriate conditions have been fulfilled. Once the conditions are right, cells are pushed through a decision point called the “restriction point” into the cell division cycle by the activity of Cdk4/6-Cyclin D complexes. Once through this point of no return, cells activate Cdk2-Cyclin E in order to drive the duplication of the DNA that will be segregated into two daughter cells later in the cell cycle (by Cdk1-Cyclin B).

In order to be able to proliferate illegitimately, cancer cells inappropriately boost the kinase activity of Cdk4/6-Cyclin D complexes or bypass the requirement for Cdk4/6-Cyclin D activation, by activating the downstream Cdk2-Cyclin E complex, independently of any input from Cdk4/6-Cyclin D. Implementation of either of these two approaches enable cancers to evade the normal controls that maintain balanced growth and homeostasis within the body. Consequently, cancer proliferation is unregulated.

Drugs that inhibit the Cdk4/6-Cyclin D complexes are having a major therapeutic impact in hormone responsive HER2 negative breast cancer (HER2-ER+) and are being trialed in a variety of other cancers.

In contrast, counteracting Cdk2-Cyclin E hyperactivation was considered to be undruggable until a recent study reported that a WEE1 family kinase called PKMYT1 is only essential when Cyclin E levels are abnormally high (Gallo et al., 2022). Importantly, they found that PKMYT1 ablation does not kill normal cells.

The same study showed that PKMYT1 ablation is synthetically lethal in the presence of Cyclin E (CCNE1) over-expression; CCNE1 overexpression drives the transcription of Cyclin B to elevate Cyclin B levels to generate so much Cdk1-Cyclin B that all the available Cdk1 inhibitory activity is required to restrain this Cdk1-CyclinB and prevent a catastrophic mitosis. Thus, CCNE1 overproduction generates a dependency on PKMYT1.

FBXW7 is a gene which encodes an E3 ligase that degrades Cyclin E. FBXW7 loss, has also been found to be synthetically lethal in the presence of PKMYT1 inhibition (Durocher et al., 2021), demonstrating that PKMYT1 drugs hold potential as first line therapy in several cancers. CCNE1 amplification has been reported in several cancer types including endometrial, ovarian, breast and gastric, ranging in frequency from 5-40%. CCNE1 amplification and/or FBXW7 mutations occur in >60% of uterine carcinosarcomas, >20% of uterine cancers, ~20% of ovarian cancers, ~18% of stomach cancers, ~14% of colorectal cancer, ~12% of bladder cancers, 11.5% of oesophageal cancers, ~11% of cervical cancers, 7.5% of sarcomas and ~7% of lung squamous cancers (Durocher et al., 2021). CCNE1 also occurs at lower levels in other cancers such as adenoid cystic carcinoma, pancreatic cancer, mesothelioma, lung adenocarcinoma, head & neck cancers, difuse large B-cells lymphoma, liver cancers and others (Gorski et al., 2020). Moreover, CCNE1 over expressing ovarian cancers are a subset of the 50% that are recombination proficient so do not benefit from PARP inhibitors (Gorski et al., 2020), highlighting the unmet need in these indications.

CCNE1 amplification is observed in the more aggressive subtypes including uterine carcinosarcoma (UCS; ~40%), uterine serous carcinoma (USC; ~25%), high-grade serous ovarian carcinoma (HGSOC; ~20%), and triple-negative breast cancer (TNBC; ~8%). CCNE1 over-expression in tumor biopsies is linked to lower overall survival compared to patients with normal Cyclin E1 levels. HGSOC patients with CCNE1 over-expression have a lower response rate to cisplatin, the current standard of care.

Similarly, FBXW7 is frequently mutated in several cancer types including uterine carcinosarcoma, endometrial, colorectal, cervical, bladder, head & neck, gastric, cancers and lung squamous cells carcinoma ranging in frequency from 5-39%. Like CCNE1 overexpression, FBXW7 driver mutations are observed in the more aggressive subtypes of endometrial cancer including UCS and USC.

Elevation of Cdk2-Cyclin E activity, via a variety of means, is also associated with resistance to Cdk4/6 inhibitors (Fassl et al., 2022); this suggests that PKMYT1 inhibition will also constitute a robust second line treatment in the cohort of HER2-ER+breast cancer patients treated with Cdk4/6 inhibitors who generally develop resistance after around 2 years of therapy. A recently discovered inhibitor of PKMYT1, RP-6306 (Szychowski et al., 2022), has shown efficacy in vivo in models of breast and ovarian cancers overexpressing CCNE1, as well as in a pancreatic PDX model with increased expression of CCNE1, alone or in combination with Gemcitabine (Gallo et al., 2022).

It has been claimed that synthetic lethality occurs in cancer cells between PKMYT1 inhibition and deficiency in protein phosphatase 2 (PP2A), in particular, regulatory subunit B alpha (PPP2R2A)(Yost et al., 2021). PPP2R2A inactivation is present in 15% of prostate adenocarcinoma, and at >5% in Ovarian serous cystadenocarcinoma, rectum adenocarcinoma, Bladder Urothelial Carcinoma, colorectal adenocarcinoma, breast invasive carcinoma, Uterine Corpus Endometrial Carcinoma, Uterine Carcinosarcoma, Liver hepatocellular carcinoma, Lung squamous cell carcinoma, lung adenocarcinoma.

PKMYT1 is a cell cycle regulating kinase, part of the WEE1 family of kinases that includes WEE1 and WEE2. WEE2 is restricted to gonads as it regulates meiosis. In contrast both PKMYT1 and WEE1 are ubiquitously expressed. PKMYT1 is localized predominantly in the endoplasmic reticulum and Golgi complex, while WEE1 is predominantly a nuclear protein. PKMYT1 is involved in the negative regulation of the CDK1-Cyclin B complex which promotes the progression of cells from G2-phase into the mitotic phase (M-phase) of the cell cycle. The biology of Cyclin E overproduction generates a need for the otherwise non-essential PKMYT1. Cyclin E accumulation boosts the transcription of cyclin B1; the potential to form active Cdk1-Cyclin B is greatly enhanced by CCNE1 overexpression. This places a far greater demand upon the Cdk1-Cyclin B inhibitory activity of WEE1 and PKMYT1 such that PKMYT1 becomes essential. Furthermore, CCNE1 overproduction stimulates abnormally high levels of DNA replication that deplete the nucleotide pool and generate DNA damage (Jones et al, 2013). The DNA damage generated by Cyclin E accumulation is not in itself lethal because cells have G2/M checkpoints that restrain commitment to genome segregation in mitosis until all damage is repaired. These checkpoint pathways boost the activity of the Wee1 family kinases WEE1 and PKMYT1, which restrain division by phosphorylating Cdk1 kinase to block Cdk1-Cyclin B activity. While damage persists, WEE1 and PKMYT1 activities remain high and cells cannot divide. Thus, WEE1 or PKMYT1 inhibition kills damaged cells by forcing them to divide when their DNA is still damaged and/or un-replicated. This places higher demands upon the ability of WEE1 and PKMYT1 to restrain CDK1-Cyclin B activity to maintain cell viability.

PKMYT1 can be removed from untransformed cells because the requirement for restraint of CDK1-CyclinB1 activity can be met by WEE1 alone. It is only when abnormally high levels of DNA damage generates a greater need for CDK1 cyclin B inhibition that PKMYT1's activities become essential.

The WEE1 inhibitor adavosertib has progressed to clinical trials in a number of solid tumours (clinicaltrials.gov) but presented significant toxicity. WEE1 inhibition toxicity most likely arises from its ability to inhibit both CDK2 and CDK1 complexes. CDK2-Cyclin E and CDK2-Cyclin A regulate the initiation and progression through DNA replication. Release of excessive levels of CDK2-Cyclin activities will generate DNA damage in a phenomenon known as oncogene induced replicative senescence. PKMYT1 inhibition is unlikely to display similar S phase toxicity, because, unlike WEE1, it phosphorylates CDK1 (Booher et al., 1997; Liu et al., 1997).

Collectively, the dependency on PKMYT1 that is generated by excessive Cdk2-Cyclin E activity in cancer cells and the markedly reduced toxicity in normal tissues arising from its restriction to CDK1 regulation make PKMYT1 is a highly attractive target for inhibition for patients whose tumours proliferate inappropriately because of enhanced Cdk2-Cyclin E activity.

Overexpression of PKMYT1 has been observed in various cancers (compared to normal tissues), including Lung squamous cell carcinoma, lung adenocarcinoma, Uterine Corpus Endometrial Carcinoma, breast invasive carcinoma, hepatocellular carcinoma, clear-cell renal-cell carcinoma, Kidney Chromophobe cancer, renal papillary cell carcinoma, Head and Neck squamous cell carcinoma, colon adenocarcinoma, stomach adenocarcinoma, thyroid carcinoma, prostate adenocarcinoma. Elevated expression of PKMYT1 is associated with poor prognosis in adrenocortical carcinoma, kidney chromophobe, kidney renal clear cell carcinoma, kidney renal papillary cell carcinoma, lower grade glioma, liver hepatocellular carcinoma, lung adenocarcinoma, mesothelioma, pancreatic adenocarcinoma, prostate adenocarcinoma, skin cutaneous melanoma, uveal melanoma (Shao et al., 2021), and breast cancer (Liu et al., 2020).

PKMYT1 is involved in the progression, invasion and/or metastasis of many solid tumours, for example non-small cell lung cancer (Zhang et al., 2022; He et al., 2021; Sun et al., 2019), osteosarcoma (Luo et al., 2022), clear cell renal cell carcinoma (Chen et al., 2020; Chen et al., 2021), oral squamous cell carcinoma (Cai et al., 2022), gastric cancer (Hu et al., 2022; Zhang et al., 2020), prostate cancer (Wang et al., 2020), oesophageal squamous cell carcinoma (Zhang et al., 2019), colorectal cancer (Jeong et al., 2018), hepatocellular carcinoma (Liu et al., 2017), ovarian cancer (Xuan et al., 2020), neuroblastoma (in particular with MYCN amplification) (Chayka et al., 2015), glioblastoma (Toledo et al., 2015). PKMYT1 is essential for survival of some haematologic malignancies, such as acute lymphoblastic leukemia and multiple myeloma (Ghelli Luserna di Rora et al., 2020).

PKMYT1 can have application in addressing resistance to treatment or improving the efficacy of cancer treatment agents. PKMYT1 elevation has been reported as a resistance mechanism to sustained WEE1 inhibition (Lewis et al., 2019). PKMYT1 inhibitors may also be a useful second line treatment to complement the emerging WEE1i based therapies.

Knockdown of PKMYT1 can eliminate the radiation-induced G2/M arrest, resulting in a lower survival rate for cells receiving radiation therapy and is therefore a promising target to improve the radiosensitivity of lung adenocarcinoma (Long et al., 2020). PKMYT1 could be also prove useful to enhance the efficacy of anti-microtubule cancer drugs (Visconti et al., 2017).

PKMYT1 also plays a role in viral infection. Knockdown of PKMYT1 reduces the number of cells supporting Kaposi sarcoma herpesvirus (KSHV) lytic infection in S phase of the cell cycle (Bryan et al., 2006). KSHV is the cause of Kaposi's sarcoma, primary effusion lymphoma (PEL) and the plasmablastic variant of multicentric Castleman's disease.

There is a clear need for PKMYT1 selective inhibitors with good pharmacokinetic properties, which are suitable for oral dosing with minimal or no toxicity.

This disclosure provides compounds and compositions that selectively inhibit PKMYT1 to treat cancer.

### SUMMARY OF THE INVENTION

One aspect of the invention pertains to certain biarylamide compounds (also referred to herein as “BAA compounds”) which inhibit Protein Kinase, Membrane Associated Tyrosine/Threonine 1 (PKMYT1), as described herein.

Another aspect of the invention pertains to a composition (e.g., a pharmaceutical composition) comprising a BAA compound, as described herein, and a pharmaceutically acceptable carrier or diluent.

Another aspect of the invention pertains to a method of preparing a composition (e.g., a pharmaceutical composition) comprising the step of mixing a BAA compound, as described herein, and a pharmaceutically acceptable carrier or diluent.

Another aspect of the present invention pertains to a method of inhibiting PKMYT1 (e.g., inhibiting or reducing or blocking the activity or function of PKMYT1), in vitro or in vivo, comprising contacting the PKMYT1 with an effective amount of a BAA compound, as described herein.

Another aspect of the present invention pertains to a method of inhibiting PKMYT1 (e.g., inhibiting or reducing or blocking the activity or function of PKMYT1) in a cell, in vitro or in vivo, comprising contacting the cell with an effective amount of a BAA compound, as described herein.

Another aspect of the present invention pertains to a BAA compound as described herein for use in a method of treatment of the human or animal body by therapy, for example, for use in a method of treatment of a disorder (e.g., a disease) as described herein.

Another aspect of the present invention pertains to use of a BAA compound as described herein in a method of treatment of the human or animal body by therapy, for example, in a method of treatment of a disorder (e.g., a disease) as described herein.

Another aspect of the present invention pertains to use of a BAA compound, as described herein, in the manufacture of a medicament, for example, for use in a method of treatment, for example, for use in a method of treatment of a disorder (e.g., a disease) as described herein.

Another aspect of the present invention pertains to a method of treatment, for example, a method of treatment of a disorder (e.g., a disease) as described herein, comprising administering to a subject in need of treatment a therapeutically-effective amount of a BAA compound, as described herein, preferably in the form of a pharmaceutical composition.

In one embodiment, the disorder is a disorder that is ameliorated by the inhibition of PKMYT1 (e.g., by the inhibition or reduction or blockage of the activity or function of PKMYT1).

In one embodiment, the disorder is, for example, a proliferative condition, cancer, etc., as described herein.

Another aspect of the present invention pertains to a kit comprising (a) a BAA compound, as described herein, preferably provided as a composition (e.g., a pharmaceutical composition) and in a suitable container and/or with suitable packaging; and (b) instructions for use, for example, in a method of treatment of a disorder (e.g., a disease) as described herein, for example, written instructions on how to administer the compound.

Another aspect of the present invention pertains to a BAA compound obtainable by a method of synthesis as described herein, or a method comprising a method of synthesis as described herein.

Another aspect of the present invention pertains to a BAA compound obtained by a method of synthesis as described herein, or a method comprising a method of synthesis as described herein.

Another aspect of the present invention pertains to novel intermediates, as described herein, which are suitable for use in the methods of synthesis described herein.

Another aspect of the present invention pertains to the use of such novel intermediates, as described herein, in the methods of synthesis described herein.

As will be appreciated by one of skill in the art, features and preferred embodiments of one aspect of the invention will also pertain to other aspects of the invention.

### DETAILED DESCRIPTION

### Compounds

One aspect of the present invention is a compound of the following formula, or a pharmaceutically acceptable salt or solvate thereof, wherein Ring A and Ring B are as defined herein (for convenience, collectively referred to herein as “biarylamide compounds” or “BAA compounds”):

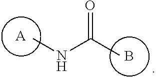

Some embodiments include the following:
(1) A compound of the following formula:

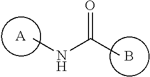

or a pharmaceutically acceptable salt or solvate thereof;
wherein:
—Ring A is:

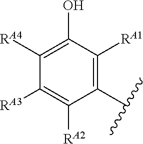

wherein:
—R^{A1} is —R^{A11};
—R^{A11} is —R^{A111}, —F, —Cl, —Br, —I, —CF₃, —CHF₂, —OH, —OR^{A111}, —OCF₃, —NH₂, NHR^{A111}, —NR^{A111}₂, —CN, —C(═O)R^{A111}, —C(═O)OH, —C(═O)OR^{A111}, —C(═O)NH₂, —C(═O)NHR^{A111}, —C(═O)NR^{A111}₂, or —S(═O)₂R^{A111};
each —R^{A111} is independently linear or branched saturated C₁₋₄alkyl; R^{A2} is —R^{A22};
—R^{A22} is —R^{A222}, —F, —Cl, —Br, —I, —CF₃, —CHF₂, —OH, —OR^{A222}, —OCF₃, —NH₂, NHR^{A222}, —NR^{A222}₂, —CN, —C(═O)R^{A222}, —C(═O)OH, —C(═O)OR^{A222}, —C(═O)NH₂, —C(═O)NHR^{A222}, —C(═O)NR^{A222}₂, or —S(═O)₂R^{A222};
each —R^{A222} is independently linear or branched saturated C₁₋₄alkyl;
—R^{A3} is —H or —R^{A33};
—R^{A33} is —R^{A333}, —F, —Cl, —Br, —I, —CF₃, —OH, —OR^{A333}, or —OCF₃;
each —R^{A333} is independently linear or branched saturated C₁₋₄alkyl;
—R^{A4} is —H or —R^{A44};
—R^{A44} is —R^{A444}, —F, —Cl, —Br, —I, —CF₃, —OH, —OR^{A444}, or —OCF₃;
each —RA444 is independently linear or branched saturated C₁₋₄alkyl;
and:
—Ring B is selected from:

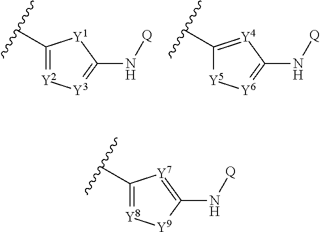

wherein:
Y¹ is S, O, NH, or NR^{Y1};
Y² is CH, CR^{Y2}, or N;
Y³ is N, CH, or CR^{Y3};
Y⁴ is N, CH, or CR^{Y4};
Y⁵ is S, O, NH, or NR^{Y5};
Y⁶ is N, CH, or CR^{Y6};
Y⁷ is N, CH, or CR^{Y7};
Y⁸ is N, CH, or CR^{Y8};
Y⁹ is S, O, NH, or NR^{Y9};
wherein:
each —R^{Y2}, —R^{Y3}, —R^{Y4}, —R^{Y6}, —R^{Y7}, and —R^{Y8} is independently —H, —F, —Cl, —Br, —I, —R^{YY}, —CF₃, —OH, —OR^{YY}, —OCF₃, —NH₂, —NHR^{YY}, or —NR^{YY}₂;
each —R^{YY} is independently linear or branched saturated C₁₋₄alkyl;
and wherein:
each —R^{Y1}, —R^{Y5}, and —R^{Y9} is independently —R^{YYN}, —C(═O)R^{YYN}, —C(═O)OR^{YYN}, —C(═O)NH₂, —C(═O)NHR^{YYN}, —C(═O)NR^{YYN}₂, or —S(═O)₂R^{YYN};
each —R^{YYN} is independently linear or branched saturated C₁₋₄alkyl, C₃-6cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected
from: —F, —Cl, —CH₃, —CF₃, —OH, —OCH₃, —NH₂,
—NH(CH₃), and —N(CH₃)₂;
and wherein:
-Q is -Q¹, -L^{Q1}-Q¹, -Q², -L^{Q2}-Q², -Q³, -L^{Q3}-Q³, -Q⁴, -L^{Q4}-Q⁴, -Q⁵, or —H;
wherein:
Q¹ is C₅₋₁₀heteroaryl;
and is:
optionally substituted on carbon with one or more groups —R^{Q1C}; and
optionally substituted on secondary nitrogen, if present, with one or more groups —R^{Q1N};
-L^{Q1}- is linear or branched saturated C₁₋₄alkylene;
Q² is C₃₋₁₀heterocyclyl;
and is:
optionally substituted on sulfur, if present, with one or two groups ═O;
optionally substituted on carbon with one or more groups —R^{Q2C}; and
optionally substituted on secondary nitrogen, if present, with one or more groups —R^{Q2N};
-L^{Q2}- is linear or branched saturated C₁₋₄alkylene;
Q³ is phenyl or naphthyl;
and is optionally substituted with one or more groups —R^{Q3C};
-L^{Q3}- is linear or branched saturated C₁₋₄alkylene;
Q⁴ is C₃₋₁₀carbocyclyl;
and is optionally substituted with one or more groups —R^{Q4C};
L^{Q4}- is linear or branched saturated C₁₋₄alkylene;
Q⁵ is linear or branched saturated C1-6alkyl;
and is optionally substituted with one or more groups —R^{Q5C};
and wherein:
each —R^{Q1C} is independently:
—F, —Cl, —Br, —I,
—R^{Q1CC},
—R^{Q1CX}, —OR^{Q1CX},
—OH, —OR^{Q1CC},
-L^{Q1C}—OH, -L^{Q1C}-OR^{Q1CC},
—NH₂, —NHR^{Q1CC}, —NR^{Q1CC}₂, —R^{Q1CM},
L^{Q1C}—NH₂, -L^{Q1C}-NHR^{Q1CC}-L^{Q1C}-NR^{Q1CC}₂, -L^{Q1C}-R^{Q1CM},
—NHC(═O)R^{Q1CC}, —N(R^{Q1CC})C(═O)R^{Q1CC}, —NHC(═O)OR^{Q1CC},
-L^{Q1C}—NHC(═O)R^{Q1CC}, -L^{Q1C}-NHC(═O)OR^{Q1CC},
—C(═O)NH₂, —C(═O)NHR^{Q1CC}, —C(═O)NR^{Q1CC}₂, —C(═O)R^{Q1CM}, ═O
—NHC(═O)NH₂, —NHC(═O)NHR^{Q1CC}, —NHC(═O)NR^{Q1CC}₂, —NHC(═O)R^{Q1CM},
-L^{Q1C}—C(═O)NH₂, -L^{Q1C}—C(═O)NHR^{Q1CC}, -L^{Q1C}-C(═O)NR^{Q1CC}₂, -L^{Q1C}-C(═O)R^{Q1CM},
—C(═O)OH, —C(═O)OR^{Q1CC},
—OC(═O)R^{Q1CC},
—OC(═O)NH₂, —OC(═O)NHR^{Q1CC}, —OC(═O)NR^{Q1CC}₂, —OC(═O)R^{Q1CM},
—S(═O)₂R^{Q1CC}, —S(═O)₂R^{Q1CX},
—S(═O)₂NH₂, —S(═O)₂NHR^{Q1CC}, —S(═O)₂NR^{Q1CC}₂, —S(═O)₂R^{Q1CM},
—CN, —C≡CH, or —NO₂;
and two adjacent —R^{Q1C}, if present, taken together may form —(CH₂)ₙ₁—O—(CH₂)ₘ₁— or
—O—(CH₂)ₚ₁—O—, wherein: n1 is 0, 1, 2, or 3; m1 is 0, 1, 2, or 3; and p1 is 1 or 2; with the proviso that m1+n1 is 2 or 3;
wherein:
each —R^{Q1CC} is independently linear or branched saturated C1-6alkyl, C₂₋₆alkenyl,
C₃₋₆cycloalkyl, C₃₋₆cycloalkyl-C₁₋₃alkyl, C₃₋₇heterocyclyl, phenyl, phenyl-C₁₋₃alkyl,
C₅₋₆heteroaryl, or C₅₋₆heteroaryl-C₁₋₃alkyl, wherein C1-6alkyl is optionally substituted
with —OH, —CN or —OCH₃, and each cycloalkyl, phenyl and heteroaryl is optionally
substituted with one or more groups selected
from: —F, —Cl, —C₁₋₄alkyl, —CHF₂, —CF₃, —OH, —OCH₃, —NH₂,
—NH(CH₃), and —N(CH₃)₂;
each —R^{Q1CX} is independently linear or branched saturated C₁₋₄haloalkyl;
each -L^{Q1C}- is independently linear or branched saturated C₁₋₄alkylene;
each —R^{Q1CM} is independently non-aromatic C₃₋₁₁heterocyclyl having at least one N ring atom, and is attached via that N ring atom; and is:
optionally substituted on carbon with one or more groups —R^{Q1CMM};
optionally substituted on sulfur, if present, with one or two ═O groups; and
optionally substituted on secondary nitrogen, if present, with a group selected from: —R^{Q1CMM}, —C(═O)R^{Q1CMM}, —C(═O)OR^{Q1CMM}, —C(═O)NH₂, —C(═O)NHR^{Q1CMM}; —C(═O)NR^{Q1CMM}₂, and —S(═O)₂R^{Q1CMM}; and
each —R^{Q1CMM} is independently —F, —NH₂, linear or branched saturated C₁₋₄alkyl, C₁₋₄alkylOC(═O)NH—, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
each —R^{Q1N} is independently:
—R^{Q1NC}, -L^{Q1N}-R^{Q1NC},
—R^{Q1NX},
—R^{Q1Nhet}-L^{Q1N}-R^{Q1Nhet},
-L^{Q1N}-OH, -L^{Q1N}-OR^{Q1NC},
-L^{Q1N}—C(═O)R^{Q1NC},
—S(═O)₂R^{Q1NC},
-L^{Q1N}-C(═O)OH, -L^{Q1N}-C(═O)OR^{Q1NC},
-L^{Q1N}-C(═O)NH₂, -L^{Q1N}-C(═O)NHR^{Q1NK}, -L^{Q1N}-C(═O)NR^{Q1NC}₂, -L^{Q1N}-C(═O)R^{Q1NP},
-L^{Q1N}-NH₂, -L^{Q1N}-NHR^{Q1NC}, -L^{Q1N}-NR^{Q1NC}₂, -L^{Q1N}-R^{Q1NM},
or -L^{Q1N}—NHC(═O)OR^{Q1NC};
wherein:
each —R^{Q1NC} is independently linear or branched saturated C₁₋₄alkyl, C₃₋₆cycloalkyl,
C₃₋₆cycloalkyl-C₁₋₃alkyl, phenyl, phenyl-C₁₋₃alkyl, C₅₋₆heteroaryl, or C₅₋₆heteroaryl-C₁₋₃alkyl,
wherein each C₁₋₄alkyl is optionally substituted by —F, —OH, —C≡N, —SO₂—CH₃, or —OCH₃,
wherein each cycloalkyl, phenyl and heteroaryl is optionally substituted with one or more groups selected from: —F, —Cl, —Br, linear or branched saturated C₁₋₄alkyl, —CHF₂, —CF₃, —OH, —OCH₃, —CH₂—O—CH₃, —OCH₂CH₃, —C(═O)—NH—phenyl, —NH₂, —N H(CH₃), and —N(CH₃)₂; wherein C₁₋₄alkyl and phenyl are independently optionally substituted by —CH₃ or —OH;
each —R^{Q1NX} is independently linear or branched saturated C₁₋₄haloalkyl;
each -L^{Q1N}- is independently linear or branched saturated C₁₋₄alkylene; wherein C₁₋₄alkylene is optionally substituted by —OH or —OMe
each —R^{Q1NM} is independently non-aromatic C₃₋₇heterocyclyl having at least one N ring atom, and is attached via that N ring atom; and is:
optionally substituted on carbon with one or more groups —R^{Q1NMM};
optionally substituted on sulfur, if present, with one or two ═O groups; and
optionally substituted on secondary nitrogen, if present, with a group selected from: —R^{Q1NMM}, —C(═O)R^{Q1NMM}, —C(═O)OR^{Q1NMM}, —C(═O)NH₂, —C(═O)NHR^{Q1NMM}, —C(═O)NR^{Q1NMM}₂, and —S(═O)₂R^{Q1NMM}; and
each —R^{Q1NMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl,
wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
each —R^{Q1Nhet} is independently non-aromatic C₃₋₇heterocyclyl; and is:
optionally substituted on sulfur, if present, with one or two groups ═O;
optionally substituted on carbon with one or more groups —R^{Q1NHH} or ═O; and
optionally substituted on secondary nitrogen, if present, with a group selected from: —R^{Q1NHH}, —C(═O)R^{Q1NJJ}, —C(═O)OR^{Q1NHH}, —C(═O)NH₂, —C(═O)NHR^{Q1NHH}, —C(═O)NR^{Q1NHH}₂, and —S(═O)₂R^{Q1NHH},
wherein:
each —R^{Q1NHH} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
—R^{Q1NJJ} is —R^{J1}, —R^{J2}, -L^{J}-R^{J2}, —R^{J3}, -L^{J}-R^{J3}, —R^{J4}, -L^{J}-R^{J4}, —R^{J5}, or -L^{J}-R^{J5};
—R^{J1} is linear or branched saturated C1-6alkyl; and is optionally substituted with one or more groups selected from: —F, —OH, —OR^{JJ}, —O-phenyl, —C(═O)OH, —C(═O)OR^{JJ}, —NH₂, —NHR^{J}, and —NR^{JJ}₂;
each —R^{J2} is independently C₃₋₆cycloalkyl; and is optionally substituted with one or more groups selected from: —F, —R^{J}, —CF₃, —OH, —OR^{J}, —NH₂, —NHR^{J}, and —NR^{J2};
each —R^{J3} is independently non-aromatic C₃₋₇heterocyclyl; and is:
optionally substituted on sulfur, if present, with one or two groups ═O;
optionally substituted on carbon with one or more groups selected from —F, —R^{J}, —CF₃, —OH, —OR^{JJ}, —NH₂, —NHR^{JJ}, and —NR^{JJ}₂; and
optionally substituted on secondary nitrogen, if present, with a group selected from: —R^{JJ}, —C(═O)R^{JJ}, —C(═O)OR^{JJ}, and —S(═O)₂R^{JJ};
each —R^{J4} is phenyl; and is optionally substituted with one or more groups selected from: —F, —Cl, —R^{J}, —CF₃, —OH, —OR^{JJ}, —NH₂, —NHR^{J}, and —NR^{JJ}₂;
each —R^{J5} is independently C₅₋₆heteroaryl; and is:
optionally substituted on carbon with one or more groups selected from —F, —R^{J}, —CF₃, —OH, —OR^{J}, —NH₂, —NHR^{J}, and —NR^{JJ}₂; and
optionally substituted on secondary nitrogen, if present, with a group selected from: —R^{J}, —C(═O)R^{JJ}, —C(═O)OR^{JJ}, and —S(═O)₂R^{JJ};
each -L′- is independently linear or branched saturated C₁₋₄alkylene, and is optionally substituted with one or more-F;
each —R^{J} is linear or branched saturated C₁₋₄alkyl;
and wherein:
—R^{Q1NK} is —R^{K1}, —R^{K2}, -L^{K}-R^{K2}, —R^{K3}, -L^{K}-R^{K3}, —R^{K4}, -L^{K}-R^{K4}, —R^{K5}, or -L^{K}-R^{K5};
—R^{K1} is linear or branched saturated C1-7alkyl; and is optionally substituted with one or more groups selected from: —F, —OH, —OR^{KK}, —OCH₂CH₂OCH₃, —O-phenyl,
—C(═O)OH, —C(═O)OR^{KK}, —NH₂, —NHR^{KK}, and —NR^{KK2};
each —R^{K2} is independently C₃₋₆cycloalkyl; and is optionally substituted with one or more groups selected from: —F, —R^{KK}, —CF₃, —OH, —OR^{KK}, —NH₂, —NHR^{KK}, and —NR^{KK2};
each —R^{K3} is independently non-aromatic C₃₋₇heterocyclyl; and is:
optionally substituted on carbon with one or more groups selected from —F, —R^{KK}, —CF₃, —OH, —OR^{KK}, —NH₂, —NHR^{KK}, and —NR^{KK2}; and
optionally substituted on secondary nitrogen, if present, with a group selected from: —R^{KK}, —C(═O)R^{KK}, —C(═O)OR^{KK}, and —S(═O)₂R^{KK};
each —R^{K4} is phenyl; and is optionally substituted with one or more groups selected from: —F, —Cl, —R^{KK}, —CF₃, —OH, —OR^{KK}, —NH₂, —NHR^{KK}, and —NR^{KK2};
each —R^{K5} is independently C₅₋₆heteroaryl; and is:
optionally substituted on carbon with one or more groups selected from —F, —R^{KK}, —CF₃, —OH, —OR^{KK}, —NH₂, —NHR^{KK}, and —NR^{KK2}; and
optionally substituted on secondary nitrogen, if present, with a group selected from: —R^{KK}, —C(═O)R^{KK}, —C(═O)OR^{KK}, and —S(═O)₂R^{KK};
each -L^{K}- is linear or branched saturated C₁₋₄alkylene, and is optionally substituted with one or more —F;
each —R^{KK} is linear or branched saturated C₁₋₄alkyl;
and wherein:
—R^{Q1NP} is non-aromatic C₃₋₁₁heterocyclyl having at least one N ring atom, and is attached via that N ring atom; and is:
optionally substituted on sulfur, if present, with one or two ═O groups;
optionally substituted on carbon with one or more groups selected from —R^{Q1NPP}, —F, —OH, —OR^{Q1NPP}, and ═O; and
optionally substituted on secondary nitrogen, if present, with a group selected from: —R^{Q1NPP}, —R^{Q1NPPX}, —C(═O)R^{Q1NPP}, —C(═O)OR^{Q1NPP}, —C(═O)NH₂, —C(═O)NHR^{Q1NPP}, —C(═O)NR^{Q1NPP}₂, and —S(═O)₂R^{Q1NPP};
each —R^{Q1NPP} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl,
wherein C₁₋₄alkyl is optionally substituted with —F, —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
—R^{Q1NPPX} is linear or branched saturated C₁₋₄haloalkyl;
and wherein:
each —R^{Q2C} is independently:
—F,
—R^{Q2CC}—R^{Q2CX},
—OH, —OR^{Q2CC}, —OR^{Q2CX},
—NH₂, —NHR^{Q2CC}, —NR^{Q2CC}₂, —R^{Q2CM},
—NHC(═O)R^{Q2CC}, —NHC(═O)OR^{Q2CC},
—C(═O)NH₂, —C(═O)NHR^{Q2CC}, —C(═O)NR^{Q2CC}₂, —C(═O)R^{Q2CM},
—C(═O)OH, —C(═O)OR^{Q2CC},
—OC(═O)R^{Q2CC},
—OC(═O)NH₂, —OC(═O)NHR^{Q2CC}, —OC(═O)NR^{Q2CG}₂, —OC(═O)R^{Q2CM}, or
═O;
wherein:
each —R^{Q2CC} is independently linear or branched saturated C₁₋₄alkyl, C₃₋₆cycloalkyl, C₃₋₆cycloalkyl-C₁₋₃alkyl, phenyl, C₃₋₇heterocyclyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
each —R^{Q2CX} is independently linear or branched saturated C₁₋₄haloalkyl;
each —R^{Q2CM} is independently non-aromatic C₃₋₁₁heterocyclyl having at least one N ring atom, and is attached via that N ring atom; and is:
optionally substituted on carbon with one or more groups —R^{Q2CMM};
optionally substituted on sulfur, if present, with one or two ═O groups; and
optionally substituted on secondary nitrogen, if present, with a group selected from: —R^{Q2CMM}, —C(═O)R^{Q2CMM}, —C(═O)OR^{Q2CMM}, —C(═O)NH₂, —C(═O)NHR^{Q2CMM}, —C(═O)NR^{Q2CMM}₂, and —S(═O)₂R^{Q2CMM}; and
each —R^{Q2CMM} is independently linear or branched saturated C₁₋₄alkyl, C₃₋₆cycloalkyl,
C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂,
—NH(CH₃), and —N(CH₃)₂;
and wherein:
each —R^{Q2N} is independently:
—R^{Q2NC},
═O, —C(═O)R^{Q2NC},
—C(═O)-L^{Q2N}—OH, —C(═O)-L^{Q2N}-OR^{Q2NC},
C(═O)-L^{Q2N}-NH₂, —C(═O)-L^{Q2N}-NHR^{Q2NC}, —C(═O)-L^{Q2N}-NR^{Q2NC}₂, —C(═O)-L^{Q2N}-R^{Q2NM},
—C(═O)OR^{Q2NC},
-L^{Q2N}-NH₂, -L^{Q2N}-NHR^{Q2NC}-L^{Q2N}-NR^{Q2NC2}, -L^{Q2N}-R^{Q2NM},
—C(═O)NH₂, —C(═O)NHR^{Q2NC}, —C(═O)NR^{Q2NC}₂, —C(═O)R^{Q2NM},
-L^{Q2N}-C(═O)NH₂, -L^{Q2N}-C(═O)NHR^{Q2NC}, -L^{Q2N}-C(═O)NR^{Q2NC}₂, -L^{Q2N}-C(═O)R^{Q2NM}, or
—S(═O)₂R^{Q2NC};
wherein:
each —R^{Q2NC} is independently linear or branched saturated C1-6alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C1-6alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
each -L^{Q2N}- is independently linear or branched saturated C₁₋₄alkylene;
each —R^{Q2NM} is independently non-aromatic C₃₋₁₁heterocyclyl having at least one N ring atom, and is attached via that N ring atom; and is:
optionally substituted on carbon with one or more groups —R^{Q2NMM}, optionally substituted on sulfur, if present, with one or two ═O groups; and
optionally substituted on secondary nitrogen, if present, with a group selected from: —R^{Q2NMM}, —C(═O)R^{Q2NMM}, —C(═O)OR^{Q2NMM}, —C(═O)NH₂, —C(═O)NHR^{Q2NMM}, —C(═O)NR^{Q2NMM}₂, and —S(═O)₂R^{Q2NMM}; and
each —R^{Q2NMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected
from: —F, —Cl, —CH₃, —CF₃, —OH, —OCH₃, —NH₂,
—NH(CH₃), and —N(CH₃)₂;
and wherein:
each —R^{Q3C} is independently:
—F, —Cl, —Br, —I,
—R^{Q3CC},
—RO3CX, —OR^{Q3CX},
—OH, —OR^{Q3CC},
-L^{Q3C}—OH, -L^{Q3C}-OR^{Q3CC},
—NH₂, —NHR^{Q3CC}, —NR^{Q3CC2}, -RQ³ CM
-L^{Q3C}-NH₂, -L^{Q3C}-NHR^{Q3CC}, -L^{Q3C}-NR^{Q3CC}₂, -L^{Q3C}-RQ³ CM
—NHC(═O)R^{Q3CC}, —NHC(═O)OR^{Q3CC},
-L^{Q3C}—NHC(═O)R^{Q3CC}, -L^{Q3C}-C(═O)OR^{Q3CC},
—C(═O)NH₂, —C(═O)NHR^{Q3CC}, —C(═O)NR^{Q3CC}₂, —C(═O)RQ³ CM
-L^{Q3C}-C(═O)NH₂, -L^{Q3C}-C(═O)NHR^{Q3CC}, -L^{Q3C}-C(═O)NR^{Q3CC}₂, -L^{Q3C}-C(═O)RQ³ CM,
—C(═O)OH, —C(═O)OR^{G3CC},
—OC(═O)R^{Q3CC},
—OC(═O)NH₂, —OC(═O)NHR^{Q3CC}, —OC(═O)NR^{Q3CC}₂, —OC(═O)RQ³ CM,
—S(═O)₂R^{Q3CC}, S(═O)₂RQ³ CM;
—CN, or —NO₂;
and two adjacent —R^{Q3C}, if present, taken together may form —(CH₂)ₙ₃—O—(CH₂)ₘ₃—, —(CH₂)ₙ₃—NH—(CH₂) m3-, —(CH₂)_{q3} (C(O)—NH—(CH₂)ᵥ₃—, —NH—(CH₂)_{q3}C(O)(CH₂) v3—O—, —NH—(CH₂)ₚ₃—NH—or —O—(CH₂)ₚ₃—O—, wherein: n3 is 0, 1, 2, or 3;
m3 is 0, 1, 2, or 3; p3 is 1 or 2; q3 is 0, 1, 2, or 3; v3 is 0, 1, 2, or 3 with the proviso that m3+n3 is 2 or 3;
wherein:
each —R^{Q3CC} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C1.3alkyl, wherein C₁₋₄alkyl is optionally substituted with —F, —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
each —R^{Q3CX} is independently linear or branched saturated C₁₋₄haloalkyl;
each -L^{Q3C}- is independently linear or branched saturated C₁₋₄alkylene;
each —RO3 CM is independently non-aromatic C₃₋₁₁heterocyclyl having at least one N ring atom, and is attached via that N ring atom; and is:
optionally substituted on carbon with one or more groups —R^{Q3CXCMM}, optionally substituted on sulfur, if present, with one or two ═O groups; and
optionally substituted on secondary nitrogen, if present, with a group selected from: —R^{Q3CCMM}, —C(═O)R^{Q3CCMM}, —C(═O)OR^{Q3CCMM}, —C(═O)NH₂, —C(═O)NHR^{Q3CCMM}, —C(═O)NR^{Q3CMM}₂, and —S(═O)₂R^{23CMM}; and
each —R^{Q3CCMM} is independently linear or branched saturated-F, C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
each —R^{Q4C} is independently:
—F,
—R^{Q4CC}, —R^{Q4CX},
—OH, —OR^{Q4CC}, —OR^{Q4CX},
—NH₂, —NHR^{Q4CC}, —NR^{Q4CC}₂, -RQ⁴ CM
—NHC(═O)R^{Q4CC}, —NHC(═O)OR^{Q4CC},
—C(═O)NH₂, —C(═O)NHR^{Q4CC}, —C(═O)NR^{Q4CC}₂, —C(═O)RQ⁴ CM
—C(═O)OH, —C(═O)OR^{Q4CC},
—OC(═O)R^{Q4CC},
—OC(═O)NH₂, —OC(═O)NHR^{Q4CC}, —OC(═O)N^{RC4CC}₂, —OC(═O)RO4 CM, or
═O;
wherein:
each —R^{Q4CC} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
each —R^{Q4CX} is independently linear or branched saturated C₁₋₄haloalkyl;
each -RQ⁴ CM is independently non-aromatic C₃₋₁₁heterocyclyl having at least one N ring atom, and is attached via that N ring atom; and is:
optionally substituted on carbon with one or more groups -RQ4CMM.
optionally substituted on sulfur, if present, with one or two ═O groups; and
optionally substituted on secondary nitrogen, if present, with a group selected from: —R^{Q4CMM}, —C(═O)R^{Q4CMM}, —C(═O)OR^{Q4CMM}, —C(═O)NH₂, —C(═O)NHRQ4CMM —C(═ONR^{Q4CMM}₂, and —S(═O)₂R^{Q4CMM};
each —R^{Q4CMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
each —R05° C. is independently:
F, —OH, —OR^{Q5CC}, —OCF₃,
—NH₂, —NHR^{Q5CC}, —NR^{Q5CC}₂, -RQ⁵ CM
—NHC(═O)R^{Q5CC}, —NHC(═O)ORQ5CC
—C(═O)NH₂, —C(═O)NHR^{Q5CC}, —C(═O)NR^{Q5CC}₂, —C(═O)RQ⁵ CM
—C(═O)OH, —C(═O)ORQ5CC
—OC(═O)RQ5CC
—OC(═O)NH₂, —OC(═O)NHR^{Q5CC}, —OC(═O)NR^{Q5CC}₂, or —OC(═O)R^{Q5CM},
wherein:
each —R^{Q5CC} is independently linear or branched saturated C₁₋₄alkyl, C₃₋₆cycloalkyl, C₃₋₆Cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
each -RQ⁵ CM is independently non-aromatic C₃₋₁₁heterocyclyl having at least one N ring atom, and is attached via that N ring atom; and is:
optionally substituted on carbon with one or more groups —R^{Q5CMM}, optionally substituted on sulfur, if present, with one or two ═O groups; and optionally substituted on secondary nitrogen, if present, with a group selected from: —R^{Q5CMM}, —C(═O)R^{Q5CM}, —C(═O)ORQ⁵ CM, —C(═O)NH₂, —C(═O)NHR^{Q5CMM}, —C(═O)NR^{Q5CMM}₂, and —S(═O)₂R^{Q5CMM},
each —R^{Q5CMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂.
   (2) A compound of the following formula:

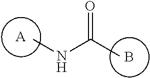

or a pharmaceutically acceptable salt or solvate thereof (e.g., or pharmaceutically acceptable salt thereof);
wherein:
Ring A is:

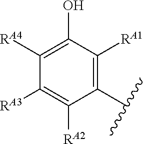

wherein:
   —R^{A1} is —R^{A11};
   —R^{A11} is —R^{A111}, —F, —Cl, —Br, —I, —CF₃, —CHF₂, —OH, —OR^{A111}, —OCF₃, —NH₂, —NHR^{A111}, —NR^{A111}₂, —CN, —C(═O)R^{A111}, —C(═O)OH, —C(═O)OR^{A111}, —C(═O)NH₂, —C(═O)NHR^{A111}, —C(═O)NR^{A111}₂, or —S(═O)₂R^{A111};
   each —R^{A111} is independently linear or branched saturated C₁₋₄alkyl; —R^{A2} is -RA22:
   —R^{A22} is —R^{A222}, —F, —Cl, —Br, —I, —CF₃, —CHF₂, —OH, —OR^{A222}, —OCF₃, —NH₂, —NHR^{A222}; —NR^{A222}₂, —CN, —C(═O)R^{A222}, —C(═O)OH, —C(═O)OR^{A222}, —C(═O)NH₂, —C(═O)NHR^{A222}, —C(═O)NR^{A222}₂, or —S(═O)₂R^{A222};
   each —R^{A222} is independently linear or branched saturated C₁₋₄alkyl;
   —R^{A3} is —H or -RA33;
   —RA33 is -RA333, —F, —Cl, —Br, —I, —CF₃, —OH, -ORA333, or —OCF₃;
   each —R^{A333} is independently linear or branched saturated C₁₋₄alkyl;
   —R^{A4} is —H or —R^{A44};
   —R^{A44} is —R^{A444}, —F, —Cl, —Br, —I, —CF₃, —OH, —OR^{A444}, or —OCF₃;
   each -RA444 is independently linear or branched saturated C₁₋₄alkyl;
and:
   Ring B is selected from:

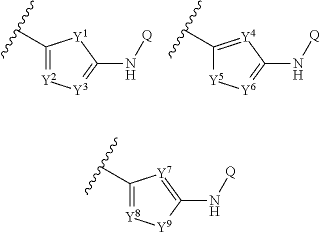

wherein:
   Y¹ is S, O, NH, NR^{Y1};
   Y² is CH, CR^{Y2}, or N;
   Y³ is N, CH, or CR^{Y3};
   Y⁴ is N, CH, or CR^{Y4};
   Y⁵ is S, O, NH, NR^{Y5};
   Y⁶ is N, CH, or CR^{Y6},
   Y⁷ is N, CH, or CR^{Y7};
   Y⁸ is N, CH, or CR^{Y8};
   Y⁹ is S, O, NH, NR^{Y9};
wherein:
   each —R^{Y2}, —R^{Y3}, —R^{Y4}, —R^{Y6}, —R^{Y7}, and —R^{Y8} is independently —H, —F, —Cl, —Br, —I, —R^{YY}, —CF₃, —OH, —OR^{YY}, —OCF₃, —NH₂, —NHR^{YY}, or —NR^{YY}₂;
   each —R^{YY} is independently linear or branched saturated C₁₋₄alkyl;
and wherein:
   each —R^{Y1}, —R^{Y5}, and —R^{Y9} is
independently —R^{YN}, —C(═O)R^{YN}, —C(═O)OR^{YN}, —C(═O)NH₂, —C(═O)NHR^{YN}, —C(═O)NR^{YN}₂, or —S(═O)₂R^{YYN};
   each —R^{YYN} is independently linear or branched saturated C₁₋₄alkyl, C₃₋₆cycloalkyl, C₃₋₆Cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
   -Q is -Q¹, -L Q1-Q¹, -Q², -LQ2-Q², -Q³, -LO3-Q³, -Q⁴, -L^{Q4}-Q⁴, -Q⁵, or —H;
wherein:
   Q¹ is C₅₋₁₀heteroaryl;
   and is:
   optionally substituted on carbon with one or more groups -RQ1C; and
   optionally substituted on secondary nitrogen, if present, with one or more groups —R^{Q1N},
   -L^{Q1}- is linear or branched saturated C₁₋₄alkylene;
   Q² is non-aromatic C₃₋₇heterocyclyl;
   and is:
   optionally substituted on sulfur, if present, with one or two groups ═O;
   optionally substituted on carbon with one or more groups —R020; and
   optionally substituted on secondary nitrogen, if present, with one or more groups —RO2N,
   -L^{Q2}- is linear or branched saturated C₁₋₄alkylene;
   -Q³ is phenyl or naphthyl;
   and is optionally substituted with one or more groups —R^{Q3C};
   -L^{Q3}- is linear or branched saturated C₁₋₄alkylene;
   Q⁴ is C₃₋₇cycloalkyl;
   and is optionally substituted with one or more groups —R^{Q4C};
   -L^{Q4}- is linear or branched saturated C₁₋₄alkylene;
   Q⁵ is linear or branched saturated C1-6alkyl;
   and is optionally substituted with one or more groups —R95C;
and wherein:
   each —R^{Q1C} is independently:
   —F, —Cl, —Br, —I,
   —R^{Q1CC},
   —R^{Q1CX}, —OR^{Q1CX},
   —OH, —OR^{Q1CC},
   L^{Q1C}—OH, -L^{Q1C}-OR^{Q1CC},
   —NH₂, —NHR^{Q1CC}, —NR^{Q1CC}₂, -RQ¹ CM
   L^{Q1C}—NH₂, -L^{Q1C}-NHR^{Q1CC}, -L^{Q1C}-NR^{Q1CC}₂, -L^{Q1C}-RQ¹ CM
   —NHC(═O)R^{Q1CC}, —NHC(═O)OR^{Q1CC},
   L^{Q1C}—NHC(═O)R^{Q1CC}, -L^{Q1C}-NHC(═O)OR^{Q1CC},
   —C(═O)NH₂, —C(═O)NHR^{Q1CC}, —C(═O)NR^{Q1CC}₂, —C(═O)R^{Q1CM},
   LIC-C(═O)NH₂, L^{Q1C}—C(═O)NHR^{Q1CC}, -L^{Q1C}-C(═O)NR^{Q1CC}₂, -LQ10-C(═O)RQ¹ CM
   —C(═O)OH, —C(═O)OR^{Q1CC},
   —OC(═O)R^{Q1CC},
   —OC(═O)NH₂, —OC(═O)NHR^{Q1CC}, —OC(═O)NR^{Q1CG}₂, —OC(═O)R^{Q1CM},
   —S(═O)₂R^{Q1CC},
   —S(═O) 2NH₂, —S(═O)₂NHR^{Q1CC}, —S(═O)₂NR°1CC2, —S(═O)₂NRQ¹ CM
   —CN, or —NO2;
   and two adjacent-RQ10, if present, taken together may form —(CH₂)ₙ₁—O—(CH₂)ₘ₁—or —O—(CH₂)ₚ₁—O—, wherein: n1 is 0, 1, 2, or 3; m1 is 0, 1, 2, or 3; and p1 is 1 or 2; with the proviso that m1+n1 is 2 or 3;
wherein:
   each —R^{Q1CC} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
   each —R^{Q1CX} is independently linear or branched saturated C₁₋₄haloalkyl;
   each -L^{Q1C}- is independently linear or branched saturated C₁₋₄alkylene;
   each —R^{Q1CM} is independently non-aromatic C₃₋₁₁heterocyclyl having at least one N ring atom, and is attached via that N ring atom; and is:
   optionally substituted on carbon with one or more groups —R^{Q1CMM};
   optionally substituted on sulfur, if present, with one or two ═O groups; and
   optionally substituted on secondary nitrogen, if present, with a group selected from: R^{Q1CMM}, —C(═O)R^{Q1CMM}, —C(═O)OR^{Q1CMM}, —C(═O)NH₂, —C(═O)NHRQ1CMM, —C(═O)NR^{Q1CMM}₂, and —S(═O)₂R^{Q1CMM}; and
   each —R^{Q1CMM} is independently linear or branched saturated C₁₋₄alkyl, C₃₋₆cycloalkyl, C₃₋₆Cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected
from: —F, —Cl, —CH₃, —CF₃, —OH, —OCH₃, —NH₂, —NH(CH₃), and —N(CH₃)₂;
and wherein:
   each —R^{Q1N} is independently:
   —R^{Q1NC},
   —RQ1NX
   —RQ1Nhet, -LQ1N-RQ1Nhet
   L^{Q1N}-OH, -L^{Q1N}-OR^{Q1NC},
   —L^{Q1N}-C(═O)R^{Q1NC},
   —L^{Q1N}-C(═O)OH, -L^{Q1N}-C(═O)OR^{Q1NC},
   —L^{Q1N}-C(═O)NH₂, -L^{Q1N}-C(═O)NHRQ1NK, -L^{Q1N}-C(═O)NR^{Q1NC}₂, -L^{Q1N}-C(═O)RQ1NP
   —L^{Q1N}-NH₂, -L^{Q1N}-NHR^{Q1NC} -L^{Q1N}-NR^{Q1NG}₂, -L^{Q1N}-R^{Q1NM},
   or -LQIN—NHC(═O)OR^{Q1N} C:
wherein:
   each —R^{Q1NC} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
   each -RQ1NX is independently linear or branched saturated C₁₋₄haloalkyl;
   each -L^{Q1N}- is independently linear or branched saturated C₁₋₄alkylene;
   each —R^{Q1NM} is independently non-aromatic C₃₋₇heterocyclyl having at least one N ring atom, and is attached via that N ring atom; and is:
   optionally substituted on carbon with one or more groups R^{Q1CMM},
   optionally substituted on sulfur, if present, with one or two ═O groups; and
   optionally substituted on secondary nitrogen, if present, with a group selected from: —R^{Q1NMM}, —C(═O)R^{Q1NMM}, —C(═O)OR^{Q1NMM}, —C(═O)NH₂, —C(═O)NHR^{Q1NMM}, —C(═O)NR°1NMM2, and —S(═O)₂R^{Q1NMM}; and
   each —R^{Q1NMM} is independently linear or branched saturated C₁₋₄alkyl, C₃₋₆cycloalkyl,
C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
   each —R^{Q1Nhet} is independently non-aromatic C₃₋₇heterocyclyl; and is:
   optionally substituted on sulfur, if present, with one or two groups ═O;
   optionally substituted on carbon with one or more groups -RQ1NHH or ═O; and
   optionally substituted on secondary nitrogen, if present, with a group selected from: —R^{Q1NHH}, —C(═O)R^{Q1NJJ}, —C(═O)OR^{Q1NHH}, —C(═O)NH₂, —C(═O)NHR^{Q1NHH}, —C(═O)NR^{Q1NHH}₂, and —S(═O)₂R^{Q1NHH},
wherein:
   each -RQ1NHH is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
   —R^{Q1NJJ} is —R^{J1}, —R^{J2}, -LJ-R^{J2}, —R^{J3}, -L^{J}-R^{J3}, —R^{J4}, -L^{J}-R^{J4}, —R^{J5}, or -L^{J}-R^{J5};
   —R^{J1} is linear or branched saturated C1-6alkyl; and is optionally substituted with one or more groups selected from: —F, —OH, —OR^{J}, —O-phenyl, —C(═O)OH, —C(═O)OR″, —NH₂, —NHR^{J}, and —NR^{J2};
   each —R^{J2} is independently C₃₋₆cycloalkyl; and is optionally substituted with one or more groups selected from: —F, —R^{J}, —CF₃, —OH, —OR^{J}, —NH₂, —NHR^{J}, and —NR^{J2};
   each —R^{J3} is independently non-aromatic C₃₋₇heterocyclyl; and is:
   optionally substituted on sulfur, if present, with one or two groups ═O;
   optionally substituted on carbon with one or more groups selected from —F, —R^{J}, —CF₃, —OH, —OR^{J}, —NH₂, —NHR^{J}, and -NRJ2; and
   optionally substituted on secondary nitrogen, if present, with a group selected from: —R^{J}, —C(═O)R^{J}, —C(═O)OR^{JJ}, and —S(═O)₂RJ;
   each —R^{J4} is phenyl; and is optionally substituted with one or more groups selected from: —F, —Cl, —R^{J}, —CF₃, —OH, —OR^{J}, —NH₂, —NHR^{J}, and —NR^{J2};
   each —R^{J5} is independently C₅₋₆heteroaryl; and is:
   optionally substituted on carbon with one or more groups selected from —F, —R^{J}, —CF₃, —OH, —OR″, —NH₂, —NHR^{J}, and -NRJ2; and
   optionally substituted on secondary nitrogen, if present, with a group selected from: —R^{J}, —C(═O)R^{J}, —C(═O)OR″, and —S(═O)₂RJ;
   each -L′- is independently linear or branched saturated C₁₋₄alkylene, and is optionally substituted with one or more-F;
   each -RJJ is linear or branched saturated C₁₋₄alkyl;
and wherein:
   R^{Q1NK} is —R^{K1}, -RK2-L^{K}-R^{K2}, -RK3-L^{K}-R^{K3}, —R^{K4}, -L^{K}-R^{K4}, —R^{K5}, or -L^{K}-R^{K5};
   —R^{K1} is linear or branched saturated C1-7alkyl; and is optionally substituted with one or more groups selected from: —F, —OH, —OR^{KK}, —OCH₂CH₂OCH₃, —O-phenyl, —C(═O)OH, —C(═O)OR^{KK}, —NH₂, —NHR^{KK}, and —NR^{KK2};
   each —R^{K2} is independently C₃₋₆cycloalkyl; and is optionally substituted with one or more groups select9*5-ed from: —F, —R^{KK}, —CF₃, —OH, —OR^{KK}, —NH₂, —NHR^{KK}, and —NR^{KK2};
   each —R^{K3} i95—** independently non-aromatic C₃₋₇heterocyclyl; and is:
   optionally substituted on carbon with one or more groups selected from —F, —R^{KK}, —CF₃, —OH, —OR^{KK}, —NH₂, —NHR^{KK}, and —NR^{KK2}; and
   optionally substituted on secondary nitrogen, if present, with a group selected from: —R^{KK}, —C(═O)R^{KK}, —C(═O)OR^{KK}, and —S(═O)₂R^{KK};
   each —R^{K4} is phenyl; and is optionally substituted with one or more groups selected from: —F, —Cl, —R^{KK}, —CF₃, —OH, —OR^{KK}, —NH₂, —NHR^{KK}, and —NR^{KK2};
   each —R^{K5} is independently C₅₋₆heteroaryl; and is:
   optionally substituted on carbon with one or more groups selected from —F, —R^{KK}, —CF₃, —OH, —OR^{KK}, —NH₂, —NHR^{KK}, and —NR^{KK2}; and
   optionally substituted on secondary nitrogen, if present, with a group selected from: —R^{KK}, —C(═O)R^{KK}, —C(═O)OR^{KK}, and —S(═O)₂R^{KK};
   each -L^{K}- is linear or branched saturated C₁₋₄alkylene, and is optionally substituted with one or more-F;
   each —R^{KK} is linear or branched saturated C₁₋₄alkyl;
and wherein:
   R^{Q1NP} is non-aromatic C₃₋₁₁heterocyclyl having at least one N ring atom, and is attached via that N ring atom; and is:
   optionally substituted on sulfur, if present, with one or two ═O groups;
   optionally substituted on carbon with one or more groups selected from —R^{Q1NPP}, —F, —OH, —OR^{Q1NPP}, and ═O; and
   optionally substituted on secondary nitrogen, if present, with a group selected from: —R^{Q1NPP}, -RQ1NPPX—C(═O)R^{Q1NPP}, —C(═O)OR^{Q1NPP}, —C(═O)NH₂, —C(═O)NHRQ1NPP —C(═O)NR^{Q1NPP}₂, and —S(═O)₂RQ1NPP:
   each —R^{Q1NPP} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
   —R^{Q1NPPX} is linear or branched saturated C₁₋₄haloalkyl;
and wherein:
   each -RQ2C is independently:
   —F,
   —RQ2CC, -RQ2CX,
   —OH, —OR^{Q2CC}, -ORQ2CX
   —NH₂, —NHR^{Q2CC}, —NR^{Q2CC}₂, —R^{Q2CM}, —NHC(═O)R^{Q2CC}, —NHC(═O)OR^{Q2CC},
   —C(═O)NH₂, —C(═O)NHR^{Q2CC}, —C(═O)NRQ20° C.2, —C(═O)R^{Q2CM},
   —C(═O)OH, —C(═O)OR^{Q2CC},
   —OC(═O)R^{Q2CC},
   —OC(═O)NH₂, —OC(═O)NHR^{Q2CC}, —OC(═O)NR02CG2, —OC(═O)R92 CM, or
   ═O;
wherein:
   each —RO2CC is independently linear or branched saturated C₁₋₄alkyl, C₃₋₆cycloalkyl, C₃₋₆cycloalkyl-C₁₋₃alkyl, phenyl, C3.7heterocyclyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
   each —R^{Q2CX} is independently linear or branched saturated C₁₋₄haloalkyl;
   each -RQ² CM is independently non-aromatic C₃₋₁₁heterocyclyl having at least one N ring atom, and is attached via that N ring atom; and is:
   optionally substituted on carbon with one or more groups -RQ2CMM.
   optionally substituted on sulfur, if present, with one or two ═O groups; and
   optionally substituted on secondary nitrogen, if present, with a group selected from: —R^{Q2CMM}, —C(═O)R92CMM, —C(═O)OR^{Q2CMM}, —C(═O)NH₂, —C(═O)NHR^{Q2CMM}, —C(═O)NR^{Q2CMM}₂, and —S(═O)₂R92CMM; and
   each -R92CMM is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
   each —R02N is independently:
   R^{Q2NC},
   —C(═O)RQ2NC
   —C(═O-L^{Q2N}—OH, —C(═O)-L^{Q2N}-ORQ2NC,
   —C(═O)-L^{Q2N}-NH₂, —C(═O)-LO2N-NHRQ2NC, —C(═O)-L^{Q2N}-NR^{Q2NC}₂, —C(═O)-L^{Q2N}-R^{Q2NM},
   —C(═O)ORQ2NC
   L^{Q2N}-NH₂, -LQ2N-NHRQ2NC-L^{Q2N}-NR^{Q2NC}₂, -LQ2N-RQ2NM,
   —C(═O)NH₂, —C(═O)NHR^{Q2NC}, —C(═O)NR^{Q2NC}₂, —C(═O)R^{Q2NM},
   L^{Q2N}-C(═O)NH₂, -L^{Q2N}-C(═O)NHR^{Q2NC}, -L^{Q2N}-C(═O)NR^{Q2NC}₂, -L^{Q2N}-C(═O)R^{Q2NM}, or
   —S(═O)₂RQ2NC;
wherein:
   each —R^{Q2NC} is independently linear or branched saturated C1-6alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C1-6alkyl is optionally substituted with —OH or —OCH₃, and each cycloalkyl, phenyl and heteroaryl is optionally substituted with one or more groups selected
from: —F, —Cl, —CH₂, —CF₃, —OH, —OCH₃, —NH₂, —NH(CH₃), and —N(CH₃)₂;
   each -LO2N- is independently linear or branched saturated C₁₋₄alkylene;
   each -RO2NM is independently non-aromatic C₃₋₁₁heterocyclyl having at least one N ring atom, and is attached via that N ring atom; and is:
   optionally substituted on carbon with one or more groups —R^{Q2NMM};
   optionally substituted on sulfur, if present, with one or two ═O groups; and
   optionally substituted on secondary nitrogen, if present, with a group selected from: —R^{Q2NMM}, —C(═O)R^{Q2NMM}, —C(═O)OR^{Q2NMM}, —C(═O)NH₂, —C(═O)NHRQ2NMM —C(═O)NRQ2NMM2, and —S(═O)₂R^{Q2NMM}; and
   each —R^{Q2NMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected
from: —F, —Cl, —CH₃, —CF₃, —OH, —OCH₃, —NH₂, —NH(CH₃), and —N(CH₃)₂;
and wherein:
   each —R23C is independently:
   —F, —Cl, —Br, —I,
   —R^{Q3CC},
   R^{Q3CX} CX, —OR^{Q3CX} CX
   OH, —OR^{Q3CC},
   L^{Q3C}—OH, -L^{Q3C}-OR^{Q3CC},
   —NH₂, —NHR^{Q3CC}, —NR^{Q3CX} CC₂, -RQ³ CM
   L^{Q3C}-NH₂, -L^{Q3C}-NHR^{Q3CX} CC-Q3C—NR^{Q3CX} CC₂, -L^{Q3C}-Q³ CM
   —NHC(═O)R93CC, —NHC(═O)OR^{Q3CC},
   L^{Q3C}—NHC(═O)R^{Q3CC}, -L^{Q3C}-C(═O)OR^{Q3CC},
   —C(═O)NH₂, —C(═O)NHR^{Q3CC}, —C(═O)NR^{Q3CXCC}₂, —C(═O)RQ³ CM,
   L^{Q3C}-C(═O)NH₂, -L^{Q3C}-C(═O)NHR^{Q3CC}, -L^{Q3C}-C(═O)NR^{Q3CXCC}₂, -L^{Q3C}-C(═O)RQ³ CM
   —C(═O)OH, —C(═O)OR^{Q3CC},
   —OC(═O)R^{Q3CC},
   —OC(═O)NH₂, —OC(═O)NHR^{Q3CC}, —OC(═O)NR^{Q3CCG}₂, —OC(═O)RQ³ CM,
   —CN, or —NO₂;
   and two adjacent-R^{Q3CX} C, if present, taken together may
form —(CH₂)ₙ₃—O—(CH₂) m3- or —O—(CH₂)ₚ₃—O—, wherein: n3 is 0, 1, 2, or 3; m3 is 0, 1, 2, or 3; and p3 is 1 or 2; with the proviso that m3+n3 is 2 or 3;
wherein:
   each —R^{Q3CC} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected
   each —R^{Q3CX} is independently linear or branched saturated C₁₋₄haloalkyl;
   each -L^{Q3C} is independently linear or branched saturated C₁₋₄alkylene;
   each —R^{Q3CM} is independently non-aromatic C₃₋₁₁heterocyclyl having at least one N ring atom, and is attached via that N ring atom; and is:
   optionally substituted on carbon with one or more groups —R^{Q3CMM};
   optionally substituted on sulfur, if present, with one or two ═O groups; and
   optionally substituted on secondary nitrogen, if present, with a group selected from: —R^{Q3CCMM}, —C(═O)ROSCMM, —C(═O)OR^{Q3CCMM}, —C(═O)NH₂, —C(═O)NHR Q3CMM —C(═O)NR^{Q3CXCMM}₂, and —S(═O)₂R^{Q3CCMM}; and
   each —R^{Q3CCMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
   each —R^{Q4C} is independently:
   F,
   —R^{Q4CC},
   RQ4CX
   OH, —OR^{Q4CC}, —OR^{Q4CX},
   —NH₂, —NHR^{Q4CC}, —NR^{Q4CC}₂, -RQ⁴ CM
   —NHC(═O)R^{Q4CC}, —NHC(═O)OR^{Q4CC},
   —C(═O)NH₂, —C(═O)NHR^{Q4CC}, —C(═O)NR^{Q4CC}₂, —C(═O)RQ⁴ CM,
   —C(═O)OH, —C(═O)OR^{Q4CC},
   —OC(═O)R^{Q4CC},
   —OC(═O)NH₂, —OC(═O)NHR^{Q4CC}, —OC(═O)NR^{Q4CC}₂, —OC(═O)RQ⁴ CM, or
   ═O;
wherein:
   each —R^{Q4CC} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
   each —R^{Q4CX} is independently linear or branched saturated C₁₋₄haloalkyl;
   each -RQ⁴ CM is independently non-aromatic C₃₋₁₁heterocyclyl having at least one N ring atom, and is attached via that N ring atom; and is:
   optionally substituted on carbon with one or more groups —R^{Q4CMM};
   optionally substituted on sulfur, if present, with one or two ═O groups; and
   optionally substituted on secondary nitrogen, if present, with a group selected from: —R^{Q4CMM}, —C(═O)R^{Q4CMM}, —C(═O)OR^{Q4CMM}, —C(═O)NH₂, —C(═O)NHRQ4CMM, —C(═ONR^{Q4CMM}₂, and —S(═O)₂RQ4CMM.
   each —R^{Q4CMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
   each —R95° C. is independently:
   —F, —OH, —OR^{Q5CC}, —OCF₃,
   —NH₂, —NHR^{Q5CC}, —NR^{Q5CC}₂, -RQ⁵ CM
   —NHC(═O)R^{Q5CC}, —NHC(═O)ORQ5CC
   —C(═O)NH₂, —C(═O)NHR^{Q5CC}, —C(═O)NR^{Q5CC}₂, —C(═O)RQ⁵ CM
   —C(═O)OH, —C(═O)ORQ5CC
   —OC(═O)R^{Q5CC},
   —OC(═O)NH₂, —OC(═O)NHR^{Q5CC}, —OC(═O)NR^{Q5CC}₂, or —OC(═O)R^{Q5CM},
wherein:
   each —R^{Q5CC} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
   each -RQ⁵ CM is independently non-aromatic C₃₋₁₁heterocyclyl having at least one N ring atom, and is attached via that N ring atom; and is:
   optionally substituted on carbon with one or more groups —R^{Q5CMM},
   optionally substituted on sulfur, if present, with one or two ═O groups; and
   optionally substituted on secondary nitrogen, if present, with a group selected from: —R^{Q5CMM}, —C(═O)R^{Q5CMM}, —C(═O)OR^{Q5CMM}, —C(═O)NH₂, —C(═O)NHR^{Q5CMM}, —C(═O)NR^{Q5CMM}₂, and —S(═O)₂R^{Q5CMM},
   each -RO5CMM is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected
from: —F, —Cl, —CH₃, —CF₃, —OH, —OCH₃, —NH₂, —NH(CH₃), and —N(CH₃)₂.
For the avoidance of doubt:

It is intended that the nitrogen atom of the central amide group, —NH—C(═O)—, linking Ring A and Ring B, is unsubstituted.

It is intended that the meta-OH substituent on Ring A is unsubstituted.

It is intended that Ring B is not fused to any other ring.

The index “C_{x-y}” in terms such as “C₉₋₁₀heteroaryl”, “C₃₋₇heterocyclyl”, and the like, refers to the number of ring atoms, which may be carbon atoms or heteroatoms (e.g., N, O, S, as the case may be). For example, pyridyl is an example of a C₆heteroaryl group, and piperidino is an example of a C₆heterocyclyl group.

The term “heteroaryl” refers to a group that is attached to the rest of the molecule by an atom that is part of an aromatic ring, wherein the aromatic ring is part of an aromatic ring system, and the aromatic ring system has one or more heteroatoms (e.g., N, O, S, as the case may be). Unless otherwise stated, the heteroaryl may be attached via a ring carbon or a ring nitrogen atom. For example, pyridyl is an example of a C₆heteroaryl group, and quinolyl (e.g., quinolin-2-yl, quinolin-7-yl, etc.) is an example of a C₁₀heteroaryl group. Furthermore, the aromatic ring system may optionally be fused with one or more non-aromatic rings which may contain one or more heteroatoms (e.g., N, O, S, as the case may be) or only carbon atoms. For example: 4,5,6,7-tetrahydro-1H-indol-2-yl is an example of a C₆heteroaryl group; 4,5,6,7-tetrahydro-1H-pyrrolo[2,3-b]pyridin-2-yl is an example of a C₆heteroaryl group. Pyridyl and furan are examples of C₅₋₆heteroaryl.

The term “heterocyclyl” refers to a group that contains at least one non aromatic ring system comprising one or more heteroatoms (e.g., N, O, S, as the case may be) that is attached to the rest of the molecule by an atom that is part of a non aromatic ring system comprising one or more heteroatoms (e.g., N, O, S, as the case may be). Unless otherwise stated, the heterocyclyl may be attached via a ring carbon or a ring nitrogen atom. In one embodiment, the term “heterocyclyl” refers to a group that is attached to the rest of the molecule by an atom that is part of a non-aromatic ring, wherein the non-aromatic ring is part of a non-aromatic ring system, and the non-aromatic ring system has one or more heteroatoms (e.g., N, O, S, as the case may be). Unless otherwise specified, a sulfur ring atom may be substituted with one or two oxo (═O) groups (for example, as in 1,1-dioxo-1,4-thiazinany-4-yl). Unless otherwise specified, “heterocyclyl” includes monocyclic heterocyclyl (e.g., piperidinyl, an example of a monocyclic C₆heterocyclyl), fused heterocyclyl (e.g., 3-azabicyclo[3.1.0]hexyl, an example of a fused C₆heterocyclyl; decahydroquinolinyl, an example of a fused C₁₀heterocyclyl), bridged heterocyclyl (e.g., 6-azabicyclo[3.1.1]heptanyl and 2,5-diazabicyclo[2.2.1]heptane, examples of a bridged C₇heterocyclyl; 3,8-diazabicyclo[3.2.1]octanyl, an example of a bridged C₈heterocyclyl), and spiro heterocyclyl(2,6-diazaspiro[3.3]heptane, an example of a spiro C₇heterocyclyl; 7-azaspiro[3.5]nonyl, an example of a spiro C₆heterocyclyl; 2,8-diazaspiro[4.5]decane, an example of a spiro C₁₀heterocyclyl). Tetrahydropyran and pyrrolidine are examples of C₃₋₇heterocyclyl. Furthermore, the non-aromatic ring system may optionally be fused with one or more aromatic rings which may contain one or more heteroatoms (e.g., N, O, S, as the case may be) or only carbon atoms. For example: 4,5,6,7-tetrahydro-1H-pyrrolo[2,3-b]pyridin-5-yl is an example of a C₆heterocyclyl group; 1,2,3,4-tetrahydroisoquinolin-3-yl is an example of a C₁₀heterocyclyl group. 2,3-Dihydro-1-benzofuran is an example of a C₉heterocyclyl group where a non-aromatic ring system is fused with an aromatic ring. 3,4-Dihydro-2H-1,4-benzoxazine and 2,3-dihydro-1,4-benzodioxine are examples of a C₁₀heterocyclyl group where a non-aromatic ring system is fused with an aromatic ring. Examples of a “non-aromatic C₃₋₇heterocyclyl” include oxetanyl, piperidine, pyrrolidine, tetrahydrofuran, tetrahydropyran, pyrrolidine, morpholinyl, azetidine and tetrahydrothiophene.

A “non-aromatic C₃₋₁₁heterocyclyl having at least one N ring atom, and is attached via that N ring atom” contains at least one non aromatic ring system comprising at least one N ring atom that is attached to the rest of the molecule by that ring N atom, and may optionally contain one or more additional heteroatoms (e.g., N, O, S, as the case may be). Furthermore, the non-aromatic ring system may optionally be fused with one or more aromatic rings which may contain one or more heteroatoms (e.g., N, O, S, as the case may be) or only carbon atoms. In one embodiment the term “non-aromatic C₃₋₁₁heterocyclyl having at least one N ring atom, and is attached via that N ring atom” refers to a heterocyclyl group that is attached via a N ring atom, but which may have additional N ring atoms. Examples include: aziridino, azetidino, pyrrolidino, piperidino, piperazino, morpholino, thiomorpholino, azepano, and diazepano.

The term “carbocyclyl” refers to a ring system that contains at least one non aromatic carbon ring, and contains only carbon atoms. Unless otherwise specified, “carbocyclyl” includes monocyclic carbocyclyl(eg cyclopropyl, cyclobutyl, cyclopentyl, cyclohexyl), bicyclic carbocyclyl(eg 2,3-dihydro-1H-indene), fused carbocyclyl(eg 7,8,9,11,12,13,14, 15, 16, 17-decahydro-6H-cyclopenta[a]phenanthrene, core scaffold of estradiol), bridged carbocyclyl(eg 1,2,3,4-tetrahydro-1,4-methanonaphthalene), and spiro carbocyclyl(eg 3′,4′-dihydro-2′H-spiro[cyclopentane-1,1′-naphthalene]).

The term “haloalkyl” refers to an alkyl group substituted with one or more halo groups (e.g., —F, —Cl, —Br, —I). The term “fluoroalkyl” refers to an alkyl group substituted with one or more-F groups. For example, —CF₃ and —CHF₂ are examples of a C₁fluoroalkyl group; —CH₂CF₃ and —CH₂CHF₂ are examples of a C₂fluoroalkyl group.

The term “C₁₋₄alkylene” refers to an alkyl group with two points of attachment. For example, —CH₂—is an example of a C₁alkylene group; —CH₂CH₂—and —CH(CH₃)—are examples of a C₂alkylene group; —CH₂CH₂CH₂— and —CH(CH₃)₂—are examples of a C₃alkylene group.

Examples of C1-7alkyl, C₁-6alkyl and C₁₋₄alkyl include methyl, ethyl, propyl, isopropyl and butyl. Examples of C₃₋₆cycloalkyl include cyclopropyl and cyclohexyl. Examples of C₃₋₆cycloalkyl-C₁₋₃alkyl include cyclopropylmethyl and cyclohexylethyl. Examples of phenyl-C₁₋₃alkyl include benzyl and 2-phenylpropyl. Examples of C₅₋₆heteroaryl-C₁₋₃alkyl include pyrimidin-2-ylmethyl and thiazol-4-ylethyl. Examples of C₂₋₆alkenyl include ethenyl and propenyl.

The phrase “one or more groups” in the context of optional substituents (e.g., “one or more groups —R^{AR1C}”, etc.) is necessarily constrained by the parent moiety and the number of positions on it that are suitable for substitution. In some parent moieties (e.g., tetrazolyl) there is only one position available for substitution. However, for other parent moieties, there may be several (e.g., phenyl has five). Except when constrained by the parent moiety, the “one or more groups” may be, e.g., 1, 2, 3, 4, etc., though more preferably is 1, 2, or 3, yet more preferably 1 or 2, still more preferably 1.

The phrase “substituent on carbon” is intended to refer to a substituent which is attached to a carbon ring atom. Similarly, the phrase “substituent on secondary nitrogen” is intended to refer to a substituent which is attached to a nitrogen ring atom which, in the absence of the substituent, would be a secondary nitrogen ring atom (i.e., —NH—). Consequently, a pyridyl group may only have “substituents on carbon”, whereas 1H-pyrrole may have both “substituents on carbon” and a “substituent on secondary nitrogen”, as illustrated below.

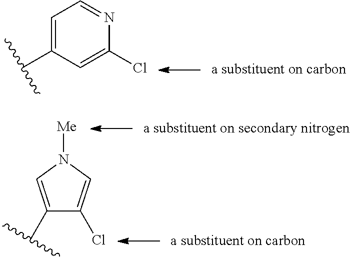 [SMILES: *c1ccnc(Cl)c1 ; *c1cn(C)cc1Cl]

Similarly, a piperidino group may only have “substituents on carbon”, whereas piperizino may have both “substituents on carbon” and a “substituent on secondary nitrogen”, as illustrated below.

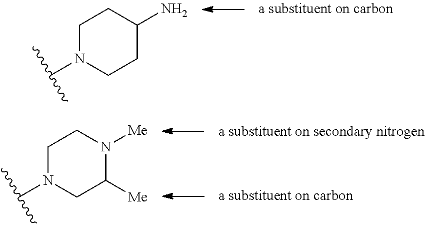 [SMILES: *N1CCC(N)CC1 ; *N1CCN(C)C(C)C1]

Certain groups despite having carbon ring atoms may not have any carbon ring atoms available for substitution. For example, a tetrazolyl group may only permit a “substituent on carbon” or may only permit a “substituent on secondary nitrogen”, as illustrated below.

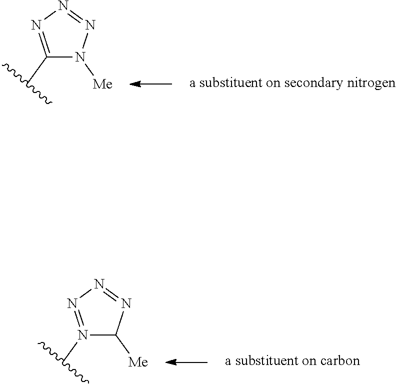

Unless otherwise indicated, where a compound is shown or described which has one or more chiral centres, and two or more stereoisomers are possible, all such stereoisomers are disclosed and encompassed, both individually (e.g., as isolated from the other stereoisomer(s)) and as mixtures (e.g., as equimolar or non-equimolar mixtures of two or more stereoisomers). For example, unless otherwise indicated, where a compound has one chiral centre, each of the (R) and(S) enantiomers are disclosed and encompassed, both individually (e.g., as isolated from the other enantiomer) and as a mixture (e.g., as equimolar or non-equimolar mixtures of the two enantiomers). For example, the initial carbon atom of a pendant sec-butyl group, —CH(CH₃) CH₂CH₃ is usually chiral, and so gives rise to stereoisomers, e.g., (R) and(S) enantiomers if it is the only chiral centre, each of which is disclosed and encompassed.

### The Group —R^{A11}

(3) A compound according to (1) or (2), wherein:
   —R^{A11} is —R^{A111}, —F, —Cl, —Br, —I, —CF₃, —CHF₂, —OH, —OR^{A111}, —OCF₃, or —CN.
(4) A compound according to (1) or (2), wherein:
   —R^{A11} is —R^{A111}, —F, —Cl, —Br, —I, —CF₃, —CHF₂, —OH, —OR^{A111}, or —OCF₃.
(5) A compound according to (1) or (2), wherein:
   —R^{A11} is —R^{A111}, —F, —Cl, —Br, —I, —CF₃, —OH, —OR^{A111}, or —OCF₃.
(6) A compound according to (1) or (2), wherein:
   —R^{A11} is —R^{A111}, —F, —Cl, —Br, —I, —OH, or —OR^{A111}.
(7) A compound according to (1) or (2), wherein:
   —R^{A11} is —R^{A111}, —F, —Cl, —Br, —I, or —OH.
(8) A compound according to (1) or (2), wherein:
   —R^{A11} is —R^{A111}, —F, —Cl, or —OH.
(9) A compound according to (1) or (2), wherein:
   —R^{A11} is —R^{A111}, —F, or —Cl.
(10) A compound according to (1) or (2), wherein:
   —R^{A11} is —R^{A111}.
(11) A compound according to (1) or (2), wherein:
   —R^{A11} is —R^{A111}, —F, —Cl, or —Br.
(12) A compound according to (1) or (2), wherein:
   —R^{A11} is —F.
(13) A compound according to (1) or (2), wherein:
   R^{A11} is —Cl.
(14) A compound according to (1) or (2), wherein:
   R^{A11} is —Br.
(15)(11) A compound according to (1) or (2), wherein:
   R^{A11} is —R^{A111}, or —Br.

### The Group —R^{A111}

(16) A compound according to any one of (1) to (15), wherein:
   —R^{A111} is -Me, -Et, -nPr, -iPr, -nBu, or -tBu.
(17) A compound according to any one of (1) to (15), wherein:
   —R^{A111} is -Me, -Et, -nPr, or -iPr.
(18) A compound according to any one of (1) to (15), wherein:
   —R^{A111} is -Me or -Et.
(19) A compound according to any one of (1) to (15), wherein:
   —R^{A111} is -Me.
(20) A compound according to any one of (1) to (15), wherein:
   —R^{A111} is -Et.

### The Group-RA22

(21) A compound according to any one of (1) to (20), wherein:
   —R^{A22} is —R^{A222}, —F, —Cl, —Br, —I, —CF₃, —CHF₂, —OH, —OR^{A222}, —OCF₃, or —CN.
(22) A compound according to any one of (1) to (20), wherein:
   —R^{A22} is —R^{A222}, —F, —Cl, —Br, —I, —CF₃, —CHF₂, —OH, —OR^{A222}, or —OCF₃.
(23) A compound according to any one of (1) to (20), wherein:
   —R^{A22} is —R^{A222}, —F, —Cl, —Br, —I, —CF₃, —OH, —OR^{A222}, or —OCF₃.
(24) A compound according to any one of (1) to (20), wherein:
   —R^{A22} is —R^{A222}, —F, —Cl, —Br, —I, —OH, or —OR^{A222}.
(25) A compound according to any one of (1) to (20), wherein:
   —R^{A22} is —R^{A222}, —F, —Cl, —Br, —I, or —OH.
(26) A compound according to any one of (1) to (20), wherein:
   —R^{A22} is —R^{A222}, —F, —Cl, or —OH.
(27) A compound according to any one of (1) to (20), wherein:
   —R^{A22} is —R^{A222}, —F, or —Cl.
(28) A compound according to any one of (1) to (20), wherein:
   —R^{A22} is -RA222.
(29) A compound according to any one of (1) to (20), wherein:
   —R^{A22} is —Cl.
(30) A compound according to any one of (1) to (20), wherein:
   —R^{A22} is —R^{A222}, —F, —Cl, or —Br.
(31) A compound according to any one of (1) to (20), wherein:
   —R^{A22} is —F.
(32) A compound according to any one of (1) to (20), wherein:
   —R^{A22} is —Br.

### The Group —R^{A222}

(33) A compound according to any one of (1) to (32), wherein:
   each —R^{A222}, if present, is linear saturated C₁₋₄alkyl.
(34) A compound according to any one of (1) to (32), wherein:
   each —R^{A222}, if present, is -Me, -Et, -nPr, -iPr, -nBu, or -tBu.
(35) A compound according to any one of (1) to (32), wherein:
   each —R^{A222}, if present, is -Me, -Et, -nPr, or -iPr.
(36) A compound according to any one of (1) to (32), wherein:
   each —R^{A222}, if present, is -Me or -Et.
(37) A compound according to any one of (1) to (32), wherein:
   each —R^{A222}, if present, is -Me.

### The Group-R^{A3}

(38) A compound according to any one of (1) to (37), wherein:
   —R^{A3} is —H.
(39) A compound according to any one of (1) to (37), wherein:
   —R^{A3} is —R^{A33} The Group —R^{A33}
(40) A compound according to any one of (1) to (37), wherein:
   —RA33, if present, is:
   —RA333, —F, —Cl, —Br, —I, —OH, or —OR^{A333}.
(41) A compound according to any one of (1) to (37), wherein:
   —R^{A33}, if present, is:
   —R^{A333}, —F, —Cl, —Br, —I, or —OH.
(42) A compound according to any one of (1) to (37), wherein:
   —R^{A33}, if present, is:
   —R^{A333}, —F, —Cl, or —OH.
(43) A compound according to any one of (1) to (37), wherein:
   —R^{A33}, if present, is:
   —R^{A333}, —F, or —Cl.
(44) A compound according to any one of (1) to (37), wherein:
   —R^{A33}, if present, is:
   —R^{A333},
(45) A compound according to any one of (1) to (37), wherein:
   —R^{A33}, if present, is:
   —Cl.
(46) A compound according to any one of (1) to (37), wherein:
   —R^{A33}, if present, is:
   —Br.

### The Group —R^{A333}

(47) A compound according to any one of (1) to (37), wherein:
   each —R^{A333}, if present, is -Me, -Et, -nPr, -iPr, -nBu, or -tBu.
(48) A compound according to any one of (1) to (37), wherein:
   each —R^{A333}, if present, is -Me, -Et, -nPr, or -iPr.
(49) A compound according to any one of (1) to (37), wherein:
   each —R^{A333}, if present, is -Me or -Et.
(50) A compound according to any one of (1) to (37), wherein:
   each —R^{A333}, if present, is -Me.

### The Group-RA4

(51) A compound according to any one of (1) to (50), wherein:
   —R^{A4} is —H.
(52) A compound according to any one of (1) to (50), wherein:
   —R^{A4} is —R^{A44}.

### The Group-R^{A44}.

(53) A compound according to any one of (1) to (52), wherein:
   —R^{A44}, if present, is:
   —R^{A44}, —F, —Cl, —Br, —I, —OH, or —OR^{A4}.
(54) A compound according to any one of (1) to (52), wherein:
   —R^{A44}, if present, is:
   —R^{A444}, —F, —Cl, —Br, —I, or —OH.
(55) A compound according to any one of (1) to (52), wherein:
   —R^{A44}, if present, is:
   —R^{A444}, —F, —Cl, or —OH.
(56) A compound according to any one of (1) to (52), wherein:
   —R^{A44}, if present, is:
   —R^{A444}, —F, or —Cl.
(57) A compound according to any one of (1) to (52), wherein:
   —R^{A44}, if present, is:
   —R^{A444}.
(58) A compound according to any one of (1) to (52), wherein:
   —R^{A44}, if present, is:
   —Cl.
(59) A compound according to any one of (1) to (52), wherein:
   —R^{A44}, if present, is:
—R^{A444}, —Cl, or —Br. The Group —R^{A444}
(60) A compound according to any one of (1) to (59), wherein:
   each —R^{A444}, if present, is linear saturated C₁₋₄alkyl.
(61) A compound according to any one of (1) to (59), wherein:
   each —R^{A444}, if present, is -Me, -Et, -nPr, -iPr, -nBu, or -tBu.
(62) A compound according to any one of (1) to (59), wherein:
   each —R^{A444}, if present, is -Me, -Et, -nPr, or -iPr.
(63) A compound according to any one of (1) to (59), wherein:
   each —R^{A444}, if present, is -Me or -Et.
(64) A compound according to any one of (1) to (59), wherein:
   each —R^{A444}, if present, is -Me.

### Ring B

(65) A compound according to any one of (1) to (64), wherein:
   Ring B is:

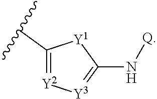

(66) A compound according to any one of (1) to (64), wherein:
   Ring B is:

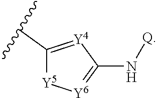

(67) A compound according to any one of (1) to (64), wherein:
   Ring B is:

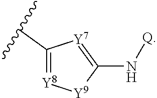

(68) A compound according to any one of (1) to (64), wherein:
   Y¹, if present, is S, O, or NH;
   Y², if present, is CH or N;
   Y³, if present, is N or CH;
   Y⁴, if present, is N or CH;
   Y⁵, if present, is S, O, or NH;
   Y⁶, if present, is N or CH;
   Y⁷, if present, is N or CH;
   Y⁸, if present, is N or CH; and
   Y⁹, if present, is S, O, or NH.

For example, wherein Ring B is selected from:

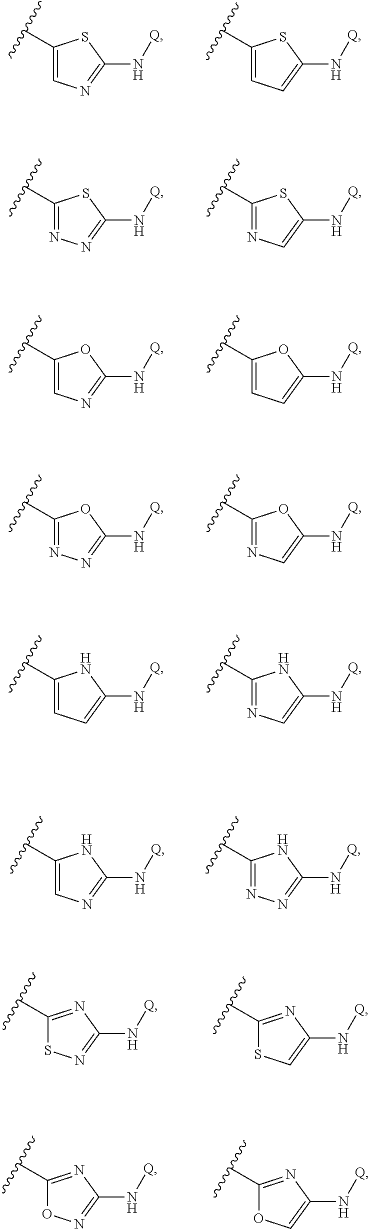

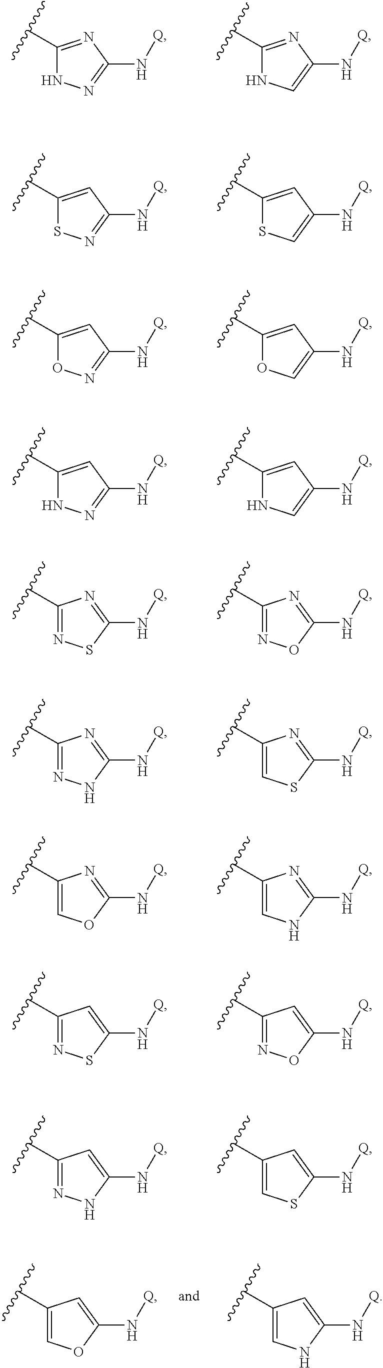

(69) A compound according to any one of (1) to (64), wherein:
   Y¹, if present, is S;
   Y², if present, is CH, CR^{Y2}, or N;
   Y³, if present, is N, CH, or CR^{Y3};
   Y⁴, if present, is N, CH, or CR^{Y4};
   Y⁵, if present, is S;
   Y⁶, if present, is N, CH, or CR^{Y6};
   Y⁷, if present, is N, CH, or CR^{Y7};
   Y⁸, if present, is N, CH, or CR^{Y8}; and
   Y⁹, if present, is S.
(70) A compound according to any one of (1) to (64), wherein:
   Y¹, if present, is S;
   Y², if present, is CH or N;
   Y³, if present, is N or CH;
   Y⁴, if present, is N or CH;
   Y⁵, if present, is S;
   Y⁶, if present, is N or CH;
   Y⁷, if present, is N or CH;
   Y⁸, if present, is N or CH; and
   Y⁹, if present, is S.
(71) A compound according to any one of (1) to (64), wherein:
   Y¹, if present, is S;
   Y², if present, is CH, CR^{Y2}, or N;
   Y³, if present, is N, CH, or CR^{Y3};
   wherein exactly one of Y² and Y³ is N;
   Y⁴, if present, is N, CH, or CR^{Y4};
   Y⁵, if present, is S;
   Y⁶, if present, is N, CH, or CR^{Y6},
   wherein exactly one of Y⁴ and Y⁶ is N;
   Y⁷, if present, is N, CH, or CR^{Y7};
   Y⁸, if present, is N, CH, or CR^{Y8};
   Y⁹, if present, is S; and
   wherein exactly one of Y⁷ and Y⁸ is N.
(72) A compound according to any one of (1) to (64), wherein Ring B is:

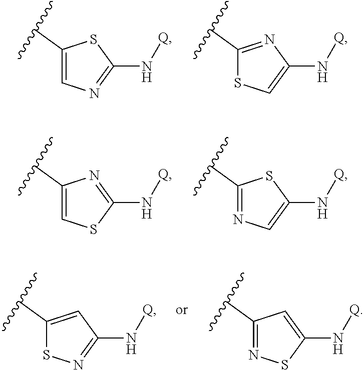

(73) A compound according to any one of (1) to (64), wherein:
   Y¹, if present, is S;
   Y², if present, is CH, CR^{Y2}, or N;
   Y³, if present, is N, CH, or CR^{Y3};
   wherein exactly one of Y² and Y³ is N;
   Y⁴, if present, is N;
   Y⁵, if present, is S;
   Y⁶, if present, is CH or CR^{Y6};
   Y⁷, if present, is N;
   Y⁸, if present, is CH or CR^{Y8}; and
   Y⁹, if present, is S.
(74) A compound according to any one of (1) to (64), wherein Ring B is:

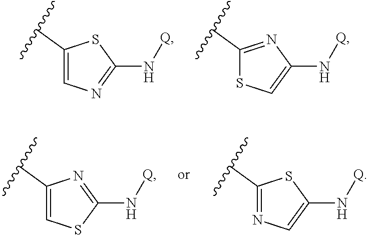

(75) A compound according to any one of (1) to (64), wherein:
   Y¹, if present, is S;
   Y², if present, is CH, CR^{Y2}, or N;
   Y³, if present, is N, CH, or CR^{Y3}; and
   wherein exactly one of Y² and Y³ is N.
(76) A compound according to any one of (1) to (64), wherein Ring B is:

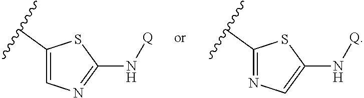

(77) A compound according to any one of (1) to (64), wherein:
   Y¹, if present, is S;
   Y², if present, is CH or CR^{Y2};
   Y³, if present, is N.
(78) A compound according to any one of (1) to (64), wherein Ring B is:

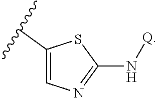

(79) A compound according to any one of (1) to (64), wherein:
   Ring B is:

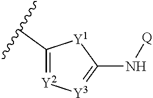

wherein:
Y¹ is S, or O;
Y² is CH, CR^{Y2}, or N;
Y³ is N.
(80) A compound according to any one of (1) to (64), wherein:
   Ring B is:

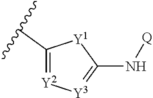

wherein:
Y¹ is S, or O;
Y² is CH, or N;
Y³ is N.
   The Groups-R^{Y2}, —R^{Y3}, etc.
(81) A compound according to any one of (1) to (80), wherein:
   each —R^{Y2}, —R^{Y3}, —R^{Y4}, —R^{Y6}, —R^{Y7}, and —R^{Y8}, if present, is independently:
   —F, —Cl, —Br, —I, —R^{YY}, —CF₃, —OH, —OR^{YY}, —OCF₃, or —NH₂.
(82) A compound according to any one of (1) to (80), wherein:
   each —R^{Y2}, —R^{Y3}, —R^{Y4}, —R^{Y6}, —R^{Y7}, and —R^{Y8}, if present, is independently:
   —F, —Cl, —Br, —I, —R^{YY}, or —NH₂.
(83) A compound according to any one of (1) to (80), wherein:
   each —R^{Y2}, —R^{Y3}, —R^{Y4}, —R^{Y6}, —R^{Y7}, and —R^{Y8}, if present, is independently:
   —F, —Cl, —R^{YY}, or —NH₂.
(84) A compound according to any one of (1) to (80), wherein:
   each —R^{Y2}, —R^{Y3}, —R^{Y4}, —R^{Y6}, —R^{Y7}, and —R^{Y8}, if present, is independently:
   —H.
(85) A compound according to any one of (1) to (80), wherein:
   each —R^{Y2}, —R^{Y3}, —R^{Y4}, —R^{Y6}, —R^{Y7}, and —R^{Y8}, if present, is independently:
   —F, —Cl, or —R^{YY}.
(86) A compound according to any one of (1) to (80), wherein:
   each —R^{Y2} is —NH₂.

### The Group —R^{YY}

(87) A compound according to any one of (1) to (86), wherein:
   each —R^{YY}, if present, is:
   -Me, -Et, -nPr, -iPr, -nBu, or -tBu.
(88) A compound according to any one of (1) to (86), wherein:
   each —R^{YY}, if present, is:
   -Me, -Et, -nPr, or -iPr.
(89) A compound according to any one of (1) to (86), wherein:
   each —R^{YY}, if present, is:
   -Me or -Et.
(90) A compound according to any one of (1) to (86), wherein:
   each —R^{YY}, if present, is:
   -Me.
      The Groups-R^{Y1}, —R^{Y5}, and —R^{Y9}
(91) A compound according to any one of (1) to (90), wherein:
   each —R^{Y1}, —R^{Y5}, and —R^{Y9}, if present, is independently:
   —R^{YN} or —C(═O)R^{YN}.
(92) A compound according to any one of (1) to (90), wherein:
   each —R^{Y1}, —R^{Y5}, and —R^{Y9}, if present, is independently:
   —R^{YN}.

### The Group-R^{YYN}

(93) A compound according to any one of (1) to (92), wherein:
   each —R^{YYN}, if present, is independently linear or branched saturated C₁₋₄alkyl, C₃₋₆cycloalkyl, C₃₋₆cycloalkyl-C₁₋₃alkyl, phenyl, phenyl-C₁₋₃alkyl, wherein each phenyl is optionally substituted with one or more groups selected from: —F, —Cl, —CH₃, —CF₃, —OH, —OCH₃, —NH₂, —NH(CH₃), and —N(CH₃)₂.
(94) A compound according to any one of (1) to (92), wherein:
   each —R^{YYN}, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(95) A compound according to any one of (1) to (92), wherein:
   each —R^{YYN}, if present, is independently linear or branched saturated C₁₋₄alkyl, phenyl, or benzyl.
(96) A compound according to any one of (1) to (92), wherein:
   each —R^{YYN}, if present, is independently linear or branched saturated C₁₋₄alkyl.
(97) A compound according to any one of (1) to (92), wherein:
   each —R^{YYN}, if present, is -Me.

### The Group-Q

(98) A compound according to any one of (1) to (97), wherein:
   -Q is -Q¹, -L^{Q1}-Q¹-Q², -L^{Q2}-Q², -Q³, -L^{Q3}-Q³, -Q⁴, -L^{Q4}-Q⁴, or -Q⁵,
(99) A compound according to any one of (1) to (97), wherein:
   -Q is -Q¹, -L^{Q1}-Q¹, -Q², or -L^{Q2}-Q².
(100) A compound according to any one of (1) to (97), wherein:
   -Q is -Q¹ or -L^{Q2}-Q²
(101) A compound according to any one of (1) to (97), wherein:
   -Q is -Q¹.
(102) A compound according to any one of (1) to (97), wherein:
   -Q is -L^{Q1}-Q¹.
(103) A compound according to any one of (1) to (97), wherein:
   -Q is -Q².
(104)(84) A compound according to any one of (1) to (97), wherein:
   -Q is -L^{Q2}-Q².
(105) A compound according to any one of (1) to (97), wherein:
   -Q is -Q³.
(106) A compound according to any one of (1) to (97), wherein:
   -Q is -L^{Q3}-Q³.
(107) A compound according to any one of (1) to (97), wherein:
   -Q is -Q⁴.
(108) A compound according to any one of (1) to (97), wherein:
   -Q is -L^{Q4}-Q⁴.
(109) A compound according to any one of (1) to (97), wherein:
   -Q is -Q⁵.
(110) A compound according to any one of (1) to (97), wherein:
   -Q is —H.

### The Group-Q¹

(111) A compound according to any one of (1) to (110), wherein:
   -Q¹, if present, is C₅₋₉heteroaryl;
   and is:
   optionally substituted on carbon with one or more groups —R^{Q1C}; and
   optionally substituted on secondary nitrogen, if present, with a group —R^{Q1N}.
(112) A compound according to any one of (1) to (110), wherein:
   -Q¹, if present, is C₅₋₆heteroaryl;
   and is:
   optionally substituted on carbon with one or more groups —R^{Q1C}; and
   optionally substituted on secondary nitrogen, if present, with a group —R^{Q1N}.
(113) A compound according to any one of (1) to (110), wherein:
   Q¹, if present, is C₅heteroaryl,
   and is:
   optionally substituted on carbon with one or more groups —R^{Q1C}; and
   optionally substituted on secondary nitrogen with a group —R^{Q1N}.
(114) A compound according to any one of (1) to (110), wherein:
   -Q¹, if present, is pyrazolyl, pyrrolyl, imidazolyl, triazolyl, tetrazolyl, furanyl, thienyl, oxazolyl, isoxazolyl, thiazolyl, isothiazolyl, oxadiazolyl, or thiadiazolyl;
   and is:
   optionally substituted on carbon with one or more groups —R^{Q1C}; and
   optionally substituted on secondary nitrogen, if present, with a group —R^{Q1N}.
(115) A compound according to any one of (1) to (110), wherein:
   -Q¹, if present, is pyrazolyl, pyrrolyl, imidazolyl, furanyl, oxazolyl, isoxazolyl, thiazolyl, isothiazolyl, oxadiazolyl, or thiadiazolyl;
   and is:
   optionally substituted on carbon with one or more groups —R^{Q1C}; and
   optionally substituted on secondary nitrogen, if present, with a group —R^{Q1N}.
(116) A compound according to any one of (1) to (110), wherein:
   -Q¹, if present, is pyrazolyl, imidazolyl, oxazolyl, isoxazolyl, thiazolyl, isothiazolyl, oxadiazolyl, or thiadiazolyl;
   and is:
   optionally substituted on carbon with one or more groups —R^{Q1C}; and
   optionally substituted on secondary nitrogen, if present, with a group —R^{Q1N}
(117) A compound according to any one of (1) to (110), wherein:
   -Q¹, if present, is pyrazolyl (e.g., 1H-pyrazol-3-yl, 1H-pyrazol-4-yl, 1H-pyrazol-5-yl, or pyrazol-1-yl);
   and is:
   optionally substituted on carbon with one or more groups —R^{Q1C}; and
   optionally substituted on secondary nitrogen, if present, with a group —R^{Q1N}.

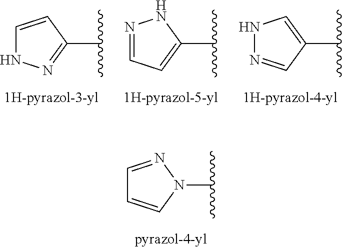

(118) A compound according to any one of (1) to (110), wherein
   -Q¹, if present, is pyrazol-1-yl;
   and is optionally substituted on carbon with one or more groups -RQ10.
(119) A compound according to any one of (1) to (110), wherein
   -Q¹, if present, is 1H-pyrazol-3-yl;
   and is:
   optionally substituted on carbon with one or more groups —R^{Q1C}; and
   optionally substituted on secondary nitrogen with a group —R^{Q1N}.
(120) A compound according to any one of (1) to (110), wherein
   -Q¹, if present, is 1H-pyrazol-4-yl;
   and is:
   optionally substituted on carbon with one or more groups -RQ10, and
   optionally substituted on secondary nitrogen with a group —R^{Q1N}.
(121) A compound according to any one of (1) to (110), wherein
   -Q¹, if present, is 1H-pyrazol-5-yl;
   and is:
   optionally substituted on carbon with one or more groups —R^{Q1C}; and
   optionally substituted on secondary nitrogen with a group —R^{Q1N}.
(122) A compound according to any one of (1) to (110), wherein:
   -Q¹, if present, is C₉heteroaryl;
   and is:
   optionally substituted on carbon with one or more groups —R^{Q1C}; and
   optionally substituted on secondary nitrogen, if present, with a group —R^{Q1N}
(123) A compound according to any one of (1) to (81105), wherein:
   -Q¹, if present, is C₉heteroaryl;
   wherein, the C₆heteroaryl is a 5:6-fused heteroaryl;
   and is:
   optionally substituted on carbon with one or more groups —R^{Q1C}; and
   optionally substituted on secondary nitrogen, if present, with a group —R^{Q1N}.
(124) A compound according to any one of (1) to (110), wherein:
   -Q¹, if present, is indolyl, indazolyl, benzimidazolyl, benzoxazolyl, pyrazolo-pyridinyl, pyrazolo-pyrimidinyl, imidazo-pyridinyl, or pyrrolo-pyridinyl;
   and is:
   optionally substituted on carbon with one or more groups —R^{Q1C}; and
   optionally substituted on secondary nitrogen, with a group —R^{Q1N}.
(125) A compound according to any one of (1) to (110), wherein:
   -Q¹, if present, is indol-2-yl or indol-3-yl;
   and is:
   optionally substituted on carbon with one or more groups —R^{Q1C}; and optionally substituted on secondary nitrogen, with a group -R^{Q1N}

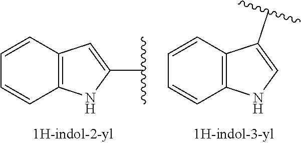

(126) A compound according to any one of (1) to (110), wherein:
   -Q¹, if present, is 2H-indazol-3-yl or 1H-indazol-3-yl;
   and is:
   optionally substituted on carbon with one or more groups —R^{Q1C}; and
   optionally substituted on secondary nitrogen, with a group —R^{Q1N}

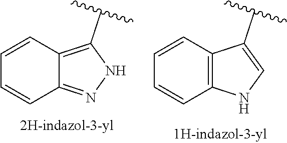 [SMILES: *c1[nH]nc2ccccc12 ; *c1c[nH]c2ccccc12]

(127) A compound according to any one of (1) to (110), wherein:
   -Q¹, if present, is benzimidazol-2-yl;
   and is:
   optionally substituted on carbon with one or more groups —R^{Q1C}; and
   optionally substituted on secondary nitrogen, with a group -R^{Q1N}

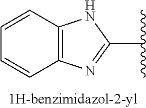 [SMILES: *c1nc2ccccc2[nH]1]

(128) A compound according to any one of (1) to (110), wherein:
   -Q¹, if present, is benzoxazol-2-yl;
   and is:
   optionally substituted on carbon with one or more groups —R^{Q1C}.

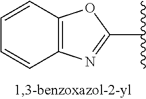 [SMILES: *c1nc2ccccc2o1]

(129) A compound according to any one of (1) to (110), wherein:
   -Q¹, if present, is pyrazolo[1,5-a]pyridin-2-yl or pyrazolo[1,5-a]pyridin-3-yl;
   and is:
   optionally substituted on carbon with one or more groups —R^{Q1C}.

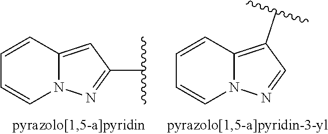

(130) A compound according to any one of (1) to (110), wherein:
   -Q¹, if present, is 1H-pyrazolo[3,4-b]pyridin-3-yl, 1H-pyrazolo[3,4-c]pyridin-3-yl, 1H-pyrazolo[4,3-c]pyridin-3-yl, or 1H-pyrazolo[4,3-b]pyridin-3-yl;
   and is:
   optionally substituted on carbon with one or more groups —R^{Q1C}; and
   optionally substituted on secondary nitrogen, with a group —R^{Q1N}

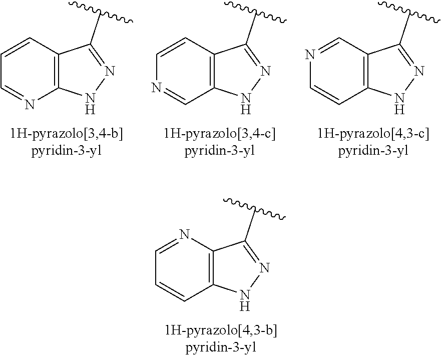 [SMILES: *c1n[nH]c2ncccc12 ; *c1n[nH]c2cnccc12 ; *c1n[nH]c2ccncc12 ; *c1n[nH]c2cccnc12]

(131) A compound according to any one of (1) to (110), wherein:
   -Q¹, if present, is imidazo[1,2-a]pyridin-2-yl or imidazo[1,2-a]pyridin-3-yl;
   and is:
   optionally substituted on carbon with one or more groups —R^{Q1C}.

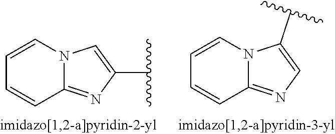

(132) A compound according to any one of (1) to (110), wherein:
   -Q¹, if present, is 1H-pyrrolo[3,2-b]pyridin-2-yl or 1H-pyrrolo[3,2-b]pyridin-3-yl;
   and is:
   optionally substituted on carbon with one or more groups —R^{Q1C}; and optionally substituted on secondary nitrogen, with a group -R^{Q1N}

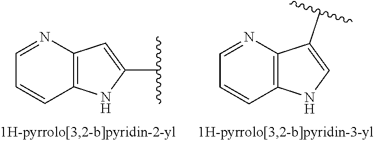 [SMILES: *c1cc2ncccc2[nH]1 ; *c1c[nH]c2cccnc12]

(133) A compound according to any one of (1) to (110), wherein:
   -Q¹, if present, is 1H-pyrrolo[3,2-c]pyridin-2-yl or 1H-pyrrolo[3,2-c]pyridin-3-yl;
   and is:
   optionally substituted on carbon with one or more groups —R^{Q1C}; and
   optionally substituted on secondary nitrogen, with a group -R^{Q1N}

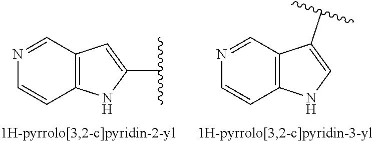 [SMILES: *c1cc2cnccc2[nH]1 ; *c1c[nH]c2ccncc12]

(134) A compound according to any one of (1) to (110), wherein:
   -Q¹, if present, is 1H-pyrrolo[2,3-c]pyridin-2-yl or 1H-pyrrolo[2,3-c]pyridin-3-yl;
   and is:
   optionally substituted on carbon with one or more groups —R^{Q1C}; and
   optionally substituted on secondary nitrogen, with a group —R^{Q1N}.

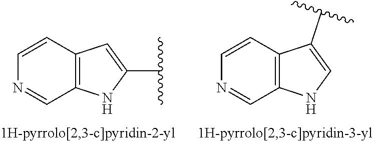 [SMILES: *c1cc2ccncc2[nH]1 ; *c1c[nH]c2cnccc12]

(135) A compound according to any one of (1) to (110), wherein:
   -Q¹, if present, is pyrazolo[1,5-c]pyrimidin-2-yl, pyrazolo[1,5-c]pyrimidin-3-yl, pyrazolo[1,5-a]pyrimidin-2-yl, or pyrazolo[1,5-a]pyrimidin-3-yl;
   and is:
   optionally substituted on carbon with one or more groups —R^{Q1C}.

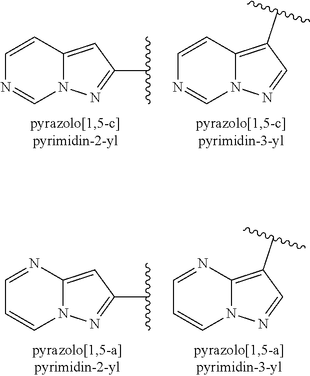

(136) A compound according to any one of (1) to (110), wherein:
   -Q¹, if present, is pyrazolyl, oxazolyl, isoxazolyl, thiazolyl, thiophenenyl, 1,3,4-oxadiazolyl, 1,2,4-oxadiazolyl, 1,3,4-thiadiazolyl, 6,7-dihydro-4H-pyrano[4,3-d]thiazolyl, imidazolyl, 1,3-benzoxazolyl, pyrazolo[1,5-a]pyridinyl, indazolyl, pyrazolo[4,3-b]pyridinyl, pyrimidinyl, pyrazolo[3,4-b]pyridinyl, pyrazolo[3,4-c]pyridinyl, 4,5,6,7-tetrahydro-1H-indazolyl, 1,4,5,6-tetrahydrocyclopenta[c]pyrazolyl, pyrazolo[4,3-c]pyridinyl, 4,5,6,7-tetrahydropyrazolo[1,5-a]pyrazinyl, 1,2,4-triazolyl, pyridinyl, pyrimidinyl, quinolinyl, isoquinolinyl, quinazolinyl, 1,8-naphthyridinyl, pyridazinyl, indolyl, 7H-pyrrolo[2,3-d]pyrimidinyl, 1,3-benzothiazolyl, thieno[2,3-d]pyrimidinyl, pyrrolo[2,1-f][1,2,4]triazinyl, benzofuranyl, 1,5-naphthyridinyl, 1,7-naphthyridinyl, 2,7-naphthyridinyl, or 1,6-naphthyridinyl;
   and is:
   optionally substituted on carbon with one or more groups —R^{Q1C}; and
   optionally substituted on secondary nitrogen, if present, with one or more groups —R^{Q1N}

### The Group-L^{Q1}-

(137) A compound according to any one of (1) to (136), wherein:
   -LQ1—, if present, is —CH₂—, —C(CH₃)₂—, —CH₂CH₂—, or —CH₂CH₂CH₂—.
(138) A compound according to any one of (1) to (136), wherein:
   -LQ1—, if present, is —CH₂—, —CH₂CH₂—, or —CH₂CH₂CH₂—.
(139) A compound according to any one of (1) to (136), wherein:
   -LQ1—, if present, is —CH₂—.
(140) A compound according to any one of (1) to (136), wherein:
   -LQ1—, if present, is —CH₂CH₂—.
(141) A compound according to any one of (1) to (136), wherein:
   -LQ1—, if present, is —CH₂CH₂CH₂—.

### The Group-Q²

(142) A compound according to any one of (1) to (141), wherein:
   -Q², if present, is azetidinyl, pyrrolidinyl, piperidinyl, piperazinyl, morpholinyl, thiomorpholinyl, azepanyl, or diazepanyl;
   and is:
   optionally substituted on sulfur, if present, with one or two groups ═O;
   optionally substituted on carbon with one or more groups -RQ20; and
   optionally substituted on secondary nitrogen, if present, with one or more groups -RQ2N
(143) A compound according to any one of (1) to (141), wherein:
   -Q², if present, is pyrrolidinyl, piperidinyl, piperazinyl, morpholinyl, or thiomorpholinyl;
   and is:
   optionally substituted on sulfur, if present, with one or two groups ═O;
   optionally substituted on carbon with one or more groups -RQ2C; and
   optionally substituted on secondary nitrogen, if present, with a group -RQ2N
(144) A compound according to any one of (1) to (141), wherein:
   -Q², if present, is piperidinyl or piperazinyl;
   and is:
   optionally substituted on carbon with one or more groups -RQ20; and
   optionally substituted on secondary nitrogen, if present, with a group —RO2N
(145) A compound according to any one of (1) to (141), wherein:
   -Q², if present, is piperidinyl (e.g., piperidin-1-yl, piperidin-2-yl, piperidin-3-yl, piperidin-4-yl);
   and is:
   optionally substituted on carbon with one or more groups —R020; and
   optionally substituted on secondary nitrogen, if present, with a group -RQ2N

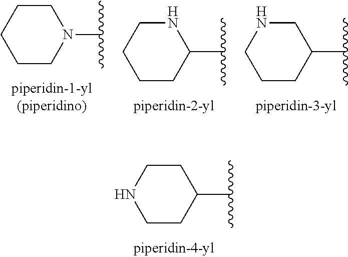

(146) A compound according to any one of (1) to (141), wherein:
   -Q², if present, is piperidin-4-yl;
   and is:
   optionally substituted on carbon with one or more groups —R^{Q2C}; and
   optionally substituted on secondary nitrogen, with a group -RQ2N
(147) A compound according to any one of (1) to (141), wherein:
   -Q², if present, is piperazinyl (e.g., piperazin-1-yl, piperazin-2-yl);
   and is:
   optionally substituted on carbon with one or more groups —R02C; and
   optionally substituted on secondary nitrogen with a group —R02N.

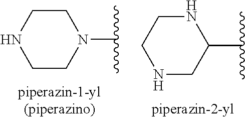

(148) A compound according to any one of (1) to (141), wherein:
   -Q², if present, is piperazin-1-yl;
   and is:
   optionally substituted on carbon with one or more groups -RQ20; and
   optionally substituted on secondary nitrogen with a group —RO2N.

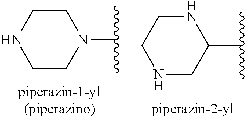

(149) A compound according to any one of (1) to (141), wherein:
-Q², if present, is pyrrolidinyl, morpholinyl, piperidinyl, thiomorpholinyl, piperazinyl, 2,3-dihydro-1,4-benzodioxinyl, 3,4-dihydro-2H-1,4-benzoxazinyl, or 2,3-dihydro-1-benzofuranyl;
   and is:
   optionally substituted on sulfur, if present, with one or two groups ═O;
   optionally substituted on secondary nitrogen, if present, with one or more groups -RQ2N

### The Group -LQ2-

(150) A compound according to any one of (1) to (149), wherein:
   -L92-, if present, is —CH₂—, —C(CH₃)₂—, —CH₂CH₂—, or —CH₂CH₂CH₂—.
(151) A compound according to any one of (1) to (149), wherein:
   L92-, if present, is —CH₂—, —CH₂CH₂—, or —CH₂CH₂CH₂—.
(152) A compound according to any one of (1) to (149), wherein:
   L92-, if present, is —CH₂—, or —CH₂CH₂CH₂—.
(153) A compound according to any one of (1) to (149), wherein:
   -L^{Q2}-, if present, is —CH₂—.
(154) A compound according to any one of (1) to (149), wherein:
   -LQ2—, if present, is —CH₂CH₂—.
(155) A compound according to any one of (1) to (149), wherein:
   -L92-, if present, is —CH₂CH₂CH₂—.

### The Group -Q³

(156) A compound according to any one of (1) to (155), wherein:
   -Q³, if present, is phenyl;
   and is optionally substituted with one or more groups —R^{Q3CX} C.
(157) A compound according to any one of (1) to (155), wherein:
   -Q³, if present, is naphthyl;
   and is optionally substituted with one or more groups —R^{Q3CX} C.

### The Group -LQ3-

(158) A compound according to any one of (1) to (157), wherein:
   -L93-, if present, is —CH₂—, —C(CH₃)₂—, —CH₂CH₂—, or —CH₂CH₂CH₂—.
(159) A compound according to any one of (1) to (157), wherein:
   -LQ3—, if present, is —CH₂—, —CH₂CH₂—, or —CH₂CH₂CH₂—.
(160) A compound according to any one of (1) to (157), wherein:
   -L^{Q3}-, if present, is —CH₂—, or —CH₂CH₂—.
(161) A compound according to any one of (1) to (157), wherein:
   -LQ3—, if present, is —CH₂—.
(162) A compound according to any one of (1) to (157), wherein:
   -L^{Q3}—, if present, is —CH₂CH₂—.
(163) A compound according to any one of (1) to (157), wherein:
   -L^{Q3}—, if present, is —CH₂CH₂CH₂—.

### The Group -Q⁴

(164) A compound according to any one of (1) to (163), wherein:
   -Q⁴, if present, is cyclopropyl, cyclobutyl, cyclopentyl, or cyclohexyl;
   and is optionally substituted with one or more groups -R^{Q4C}.
(165) A compound according to any one of (1) to (163), wherein:
   -Q⁴, if present, is cyclopropyl;
   and is optionally substituted with one or more groups -RQ4C
(166) A compound according to any one of (1) to (163), wherein:
   -Q⁴, if present, is cyclobutyl;
   and is optionally substituted with one or more groups -RQ4C
(167) A compound according to any one of (1) to (163), wherein:
   -Q⁴, if present, is cyclopentyl;
   and is optionally substituted with one or more groups -RQ4C
(168) A compound according to any one of (1) to (163), wherein:
   -Q⁴, if present, is cyclohexyl;
   and is optionally substituted with one or more groups -R^{Q4C}.
(169) A compound according to any one of (1) to (163), wherein:
   -Q⁴, if present, is cyclopropyl, or cyclohexyl;
   and is optionally substituted with one or more groups -R^{Q4C}.

### The Group -LQ4-

(170) A compound according to any one of (1) to (169), wherein:
   -L04-, if present, is —CH₂—, —C(CH₃)₂—, —CH₂CH₂—, or —CH₂CH₂CH₂—.
(171) A compound according to any one of (1) to (169), wherein:
   -LQ4—, if present, is —CH₂—, —CH₂CH₂—, or —CH₂CH₂CH₂—.
(172) A compound according to any one of (1) to (169), wherein:
   -LQ4—, if present, is —CH₂—.
(173) A compound according to any one of (1) to (169), wherein:
   -LQ4—, if present, is —CH₂CH₂—.
(174) A compound according to any one of (1) to (169), wherein:
   -LQ4, if present, is —CH₂CH₂CH₂—.

### The Group -Q⁵

(175) A compound according to any one of (1) to (174), wherein:
   -Q⁵, if present, is -Me, -Et, -nPr, -iPr, -nBu, -iBu, -sBu, -tBu, n-pentyl, iso-pentyl, neo-pentyl, n-hexyl, iso-hexyl, or 3,3-dimethylbutyl;
   and is optionally substituted with one or more groups -RQ50.
(176) A compound according to any one of (1) to (174), wherein:
   -Q⁵, if present, is linear or branched saturated C₁₋₄alkyl;
   and is optionally substituted with one or more groups -RQ50.
(177) A compound according to any one of (1) to (174), wherein:
   -Q⁵, if present, is -Me, -Et, or -nPr;
   and is optionally substituted with one or more groups -RQ5° C.
(178) A compound according to any one of (1) to (174), wherein:
   -Q⁵, if present, is —CH₂-RQ5° C., —CH₂CH₂-RQ5C, or —CH₂CH₂CH₂-R^{Q5C}.
(179) A compound according to any one of (1) to (174), wherein:
   -Q⁵, if present, is —CH₂-RQ5C
(180) A compound according to any one of (1) to (174), wherein:
   -Q⁵, if present, is —CH₂CH₂-R^{Q5C}.
(181) A compound according to any one of (1) to (174), wherein:
   -Q⁵, if present, is —CH₂CH₂CH₂—R^{Q5C}.
(182) A compound according to any one of (1) to (174), wherein:
   -Q⁵, if present, is -Me, -Et, -nPr or 3,3-dimethylbutyl. and is optionally substituted with one or more groups -RQ5C

### The Group -RQ1C

(183) A compound according to any one of (1) to (182), wherein:
   each —R^{Q1C}, if present, is independently:
   —F, —Cl, —Br, —I,
   —RQ1CC
   —R^{Q1CX}, —OR^{Q1CX},
   —OH, —OR^{Q1CC},
   -L^{Q1C}—OH, -L^{Q1C}-OR^{Q1CC}, —NH₂, —NHR^{Q1CC}, —NR^{Q1CC}₂, -RQ¹ CM
   -L^{Q1C}—NH₂, -L^{Q1C}-NHR^{Q1CC}, -L^{Q1C}-NR^{Q1CC}₂, -L^{Q1C}-RQ¹ CM
   —C(═O)NH₂, —C(═O)NHR^{Q1CC}, —C(═O)NR^{Q1CC}₂, —C(═O)RQ¹ CM
   —C(═O)OH, or —C(═O)OR^{Q1CC},
   —S(═O)₂R^{Q1CC}, or —CN;
   and two adjacent-RQ10, if present, taken together may
form —(CH₂)ₙ₁—O—(CH₂)ₘ₁—or —O—(CH₂) p1—O—.
(184) A compound according to any one of (1) to (182), wherein:
   each —R^{Q1C}, if present, is independently:
   —F, —Cl, —Br, —I,
   R^{Q1CX}, —OR^{Q1CX},
   —OH, —OR^{Q1CC},
   —NH₂, —NHR^{Q1CC}, —NR^{Q1CC}₂, -RQ¹ CM
   —C(═O)NH₂, —C(═O)NHR^{Q1CC}, —C(═O)NR^{Q1CC}₂, —C(═O)RQ¹ CM
   —C(═O)OH, or —C(═O)ORQ1CC.
(185) A compound according to any one of (1) to (182), wherein:
   each —R^{Q1C}, if present, is independently:
   —R^{Q1CC},
   —R^{Q1CX},
   —NH₂, —NHR^{Q1CC}, —NR^{Q1CC}₂, -RQ¹ CM
   —C(═O)NH₂, —C(═O)NHR^{Q1CC}, —C(═O)NR^{Q1CC}₂, —C(═O)RQ¹ CM
   —C(═O)OH, or —C(═O)OR^{Q1CC},
(186) A compound according to any one of (1) to (182), wherein:
   each —R^{Q1C}, if present, is independently:
   —R^{Q1CC},
   —R^{Q1CX}, or
   —C(═O)OR^{Q1CC},
(187) A compound according to any one of (1) to (182), wherein:
   each -RQIC, if present, is:
   —RQ1CC.
(188) A compound according to any one of (1) to (182), wherein:
   each -RQIC, if present, is independently:
   F, —Cl, —Br,
   —R^{Q1CX}, —OR^{Q1CX},
   —OH, —OR^{Q1CC}, —NH₂, -NHRQiCC, —NR^{Q1CC}₂, -RQ¹ CM
   L^{Q1C}-RQ¹ CM
   —NHC(═O)R^{Q1CC}, —N(R^{Q1CC})C(═O)R^{Q1CC}, —NHC(═O)OR^{Q1CC},
   —C(═O)NH₂, —C(═O)NHR^{Q1CC}, —C(═O)NR^{Q1CC}₂, —C(═O)R^{Q1CM}, ═O,
   —NHC(═O)NHR^{Q1CC},
   L^{Q1C}—C(═O)NRQ1CC2
   —C(═O)OH, —C(═O)OR^{Q1CC},
   —OC(═O)NH₂,
   —S(═O)₂RQ1CX
   —S(═O)₂RQ¹ CM
   NHS(═O)R^{Q1CC}, —NHS(═O)₂R^{Q1CC},
   —CN, or —C≡CH.
(189) A compound according to any one of (1) to (182), wherein:
   each —R^{Q1C}, if present, is independently:
   F, —Cl,
   —R^{Q1CC},
   —R^{Q1CX},
   OR^{Q1CC}, —NR^{Q1CC}₂, —R^{Q1CM},
   —NHC(═O)R^{Q1CC}, or
   L^{Q1C}—C(═O)NRQ1CC2.

### The Group -RQ1CC

(190) A compound according to any one of (1) to (189), wherein:
   each —R^{Q1CC}, if present, is independently linear or branched saturated C₁₋₄alkyl, C₃₋₆cycloalkyl, C₃₋₆cycloalkyl-C1.3alkyl, phenyl, phenyl-C₁₋₃alkyl, wherein each phenyl is optionally substituted with one or more groups selected from: —F, —Cl, —CH₃, —CF₃, —OH, —OCH₃, —NH₂, —NH(CH₃), and —N(CH₃)₂.
(191) A compound according to any one of (1) to (189), wherein:
   each —R^{Q1CC}, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(192) A compound according to any one of (1) to (189), wherein:
   each —R^{Q1CC}, if present, is independently linear or branched saturated C₁₋₄alkyl, phenyl, or benzyl.
(193) A compound according to any one of (1) to (189), wherein:
   each -Ro1CC, if present, is independently linear or branched saturated C₁₋₄alkyl.
(194) A compound according to any one of (1) to (189), wherein:
   each —R^{Q1CC}, if present, is independently -Me, -Et, -nPr, -iPr, -nBu, or -tBu.
(195) A compound according to any one of (1) to (189), wherein:
   each —RO1CC, if present, is independently -Me, -Et, -nPr, or -iPr.
(196) A compound according to any one of (1) to (189), wherein:
   each —RO1CC, if present, is independently -Me or -Et.
(197) A compound according to any one of (1) to (189), wherein:
   each —R^{Q1CC}, if present, is -Me.
(198) A compound according to any one of (1) to (189), wherein:
   each —R^{Q1CC}, if present, is independently linear saturated C1-6alkyl, C₂₋₆alkenyl, C₃₋₆cycloalkyl, C₃₋₇heterocyclyl, phenyl, or C₅₋₆heteroaryl, wherein C1-6alkyl is optionally substituted with —OH, —CN, or —OCH₃, and each, phenyl and heteroaryl is optionally substituted with one or more groups selected from: —F, —Cl, —CH₃, —CHF₂, —CF₃, and —OCH₃.
(199) A compound according to any one of (1) to (189), wherein:
   each —R^{Q1CC}, if present, is independently -Me, -Et, -iPr, -iBu, -t-Bu, -heptyl, C₂₋₆alkenyl, cyclopropyl, tetrahydropyran, phenyl, or pyrazolyl, pyrimidinyl, wherein-Me, -Et, -iPr, -iBu, -t-Bu, or -heptyl is optionally substituted with —OH, —CN, or —OCH₃, and each, phenyl, pyrazolyl, and, pyrimidinyl is optionally substituted with one or more groups selected from: —F, —Cl, —CH₃, —CHF₂, —CF₃, and —OCH₃.
(200) A compound according to any one of (1) to (189), wherein:
each —R^{Q1CC}, if present, is independently linear C1-6alkyl, C₃₋₆cycloalkyl, phenyl, or C₅₋₆heteroaryl, wherein each cycloalkyl, phenyl and heteroaryl is optionally substituted with one or more groups selected from: —F, —Cl, —C₁₋₄alkyl-CHF₂, —CF₃, and —OCH₃.
   The Indices “n1” and “m” in-(CH₂) 11-O—(CH₂)ₘ₁—
(201) A compound according to any one of (1) to (200), wherein:
   n1, if present, is 0, 1, 2, or 3;
   m1, if present, is 0, 1, 2, or 3;
   with the proviso that m1+n1 is 2 or 3.
(202) A compound according to any one of (1) to (200), wherein:
   n1, if present, is 0, 1, or 2;
   m1, if present, is 0, 1, or 2;
   with the proviso that m1+n1 is 2 or 3.
(203) A compound according to any one of (1) to (200), wherein:
   n1, if present, is 1 or 2;
   m1, if present, is 1 or 2;
   with the proviso that m1+n1 is 2 or 3.

The Index “p1” in —O—(CH₂) p1-O—
(204) A compound according to any one of (1) to (203), wherein:
   p1, if present, is 1.
(205) A compound according to any one of (1) to (203), wherein:
   p1, if present, is 2.

### The Group -RQ1CX

(206) A compound according to any one of (1) to (203), wherein:
   each —R^{Q1CX}, if present, is independently linear or branched saturated C₁₋₄fluoroalkyl.
(207) A compound according to any one of (1) to (203), wherein:
   each —R^{Q1CX}, if present, is independently —CF₃, —CHF₂, —CH₂CF₃, or —CH₂CHF₂.
(208) A compound according to any one of (1) to (203), wherein:
   each —R^{Q1CX}, if present, is —CF₃.
(209) A compound according to any one of (1) to (203), wherein:
   each —R^{Q1CX}, if present, is independently —CF₃, or —CH₂CF₃.

### The Group -L^{Q1C}-

(210) A compound according to any one of (1) to (209), wherein:
   each -L^{Q1C}-, if present, is independently —CH₂—, —C(CH₃)₂—, —CH₂CH₂—, or —CH₂CH₂CH₂—.
(211) A compound according to any one of (1) to (209), wherein:
   each -LO1C, if present, is independently —CH₂—, —CH₂CH₂—, or —CH₂CH₂CH₂—.
(212) A compound according to any one of (1) to (209), wherein:
   each -L^{Q1C}-, if present, is —CH₂—.
(213) A compound according to any one of (1) to (209), wherein:
   each -L^{Q1C}-, if present, is —CH₂CH₂—.
(214) A compound according to any one of (1) to (209), wherein:
   each -L^{Q1C}-, if present, is —CH₂CH₂CH₂—.

### The Group -RQ¹ CM

(215) A compound according to any one of (1) to (214), wherein:
   each —R^{Q1CM}, if present, is independently azetidino, pyrrolidino, piperidino, piperazino, morpholino, thiomorpholino, azepano, or diazepano, and is:
   optionally substituted on carbon with one or more groups R^{Q1CMM}, and
   optionally substituted on secondary nitrogen, if present, with a group selected from: R^{Q1CMM}, —C(═O)R^{Q1CMM}, —C(═O)OR^{Q1CMM}, —C(═O)NH₂, —C(═O)NHRQ1CMM, —C(═O)NR^{Q1CMM}₂, and —S(═O)₂RQ1CMM
(216) A compound according to any one of (1) to (214), wherein:
   each —R^{Q1CM}, if present, is independently pyrrolidino, piperidino, piperazino, or morpholino, and is:
   optionally substituted on carbon with one or more groups -RQICMM, and
   optionally substituted on secondary nitrogen, if present, with a group selected from: R^{Q1CMM}, —C(═O)R^{Q1CMM}, —C(═O)OR^{Q1CMM}, —C(═O)NH₂, —C(═O)NHRQ1CMM, —C(═O)NR^{Q1CMM}₂, and —S(═O)₂RQ1CMM
(217) A compound according to any one of (1) to (214), wherein:
   each —R^{Q1CM}, if present, is independently pyrrolidino, piperidino, piperazino, or morpholino, and is:
   optionally substituted on carbon with one or more groups —R^{Q1CMM}; and
   optionally substituted on secondary nitrogen, if present, with a group selected from: R^{Q1CMM}, —C(═O)R^{Q1CMM}, and —C(═O)ORQ1CMM
(218) A compound according to any one of (1) to (214), wherein:
   each —R^{Q1CM}, if present, is independently non-aromatic C₃₋₁₁heterocyclyl having at least one
   N ring atom, and is attached via that N ring atom; and is:
   optionally substituted on carbon with one or more groups —R^{Q1CMM};
   optionally substituted on secondary nitrogen, if present, with a group selected
   from: -RQ1CMM
(219) A compound according to any one of (1) to (214), wherein:
   each —R^{Q1CM}, if present, is independently piperazinyl, piperidinyl, pyrrolidinyl, morpholinyl, attached via a N ring atom; and is:
   optionally substituted on carbon with one or more groups —R^{Q1CMM};
   optionally substituted on secondary nitrogen, if present, with a group selected from: —R^{Q1CMM};
(220) A compound according to any one of (1) to (214), wherein:
   each —R^{Q1CM}, if present, is optionally substituted on secondary nitrogen, if present, with a group selected from: -RQ1CMM

### The Group -RQ1CMM

(221) A compound according to any one of (1) to (220), wherein:
   each R^{Q1CMM}, if present, is independently linear or branched saturated C₁₋₄alkyl, C₃₋₆cycloalkyl, C₃₋₆cycloalkyl-C₁₋₃alkyl, phenyl, or phenyl-C₁₋₃alkyl, wherein C₁₋₄alkyl is optionally substituted with —OH or —OCH₃, and each cycloalkyl or phenyl is optionally substituted with one or more groups selected from: —F, —Cl, —CH₃, —CF₃, —OH, —OCH₃, —NH₂, —NH(CH₃), and —N(CH₃)₂.
(222) A compound according to any one of (1) to (220), wherein:
   each —R^{Q1CMM}, if present, is independently linear or branched saturated C₁₋₄alkyl, or phenyl-C₁₋₃alkyl, wherein C₁₋₄alkyl is optionally substituted with —OH or —OCH₃, and each cycloalkyl and phenyl is optionally substituted with one or more groups selected from: —F, —Cl, —CH₃, —CF₃, —OH, —OCH₃, —NH₂, —NH(CH₃), and —N(CH₃)₂;
(223) A compound according to any one of (1) to (220), wherein:
   each —R^{Q1CMM}, if present, is independently linear or branched saturated C₁₋₄alkyl, phenyl, or phenyl-CH₂—, wherein C₁₋₄alkyl is optionally substituted with —OH or —OCH₃.
(224) A compound according to any one of (1) to (220), wherein:
   each —R^{Q1CMM}, if present, is independently linear or branched saturated C₁₋₄alkyl, phenyl, or benzyl.
(225) A compound according to any one of (1) to (220), wherein:
   each R^{Q1CMM}, if present, is independently linear or branched saturated C₁₋₄alkyl, and is optionally substituted with —OH or —OCH₃.
(226) A compound according to any one of (1) to (220), wherein:
   each —R^{Q1CMM}, if present, is independently linear or branched saturated C₁₋₄alkyl.
(227) A compound according to any one of (1) to (220), wherein:
   each —R^{Q1CMM}, if present, is independently -Me, -Et, -nPr, -iPr, -nBu, or -tBu.
(228) A compound according to any one of (1) to (220), wherein:
   each —R^{Q1CMM}, if present, is independently -Me, -Et, -nPr, or -iPr.
(229) A compound according to any one of (1) to (220), wherein:
   each —R^{Q1CMM}, if present, is independently -Me or -Et.
(230) A compound according to any one of (1) to (220), wherein:
   each —R^{Q1CMM}, if present, is -Me.
(231) A compound according to any one of (1) to (220), wherein:
   each R^{Q1CMM}, if present, is independently —F, —NH₂, linear saturated C₁₋₄alkyl,
   C₁₋₄alkyIOC(═O)NH—, or C₃₋₆cycloalkyl, wherein C₁₋₄alkyl is optionally substituted with —OH.
(232) A compound according to any one of (1) to (220), wherein:
   each —R^{Q1CMM}, if present, is independently —F, —NH₂, -Me, -Et, EtOC(═O)NH—, or cyclopropyl, wherein-Me or -Et are optionally substituted with —OH.
(233) A compound according to any one of (1) to (220), wherein:
each —R^{Q1CMM}, if present, is independently —F, or linear or branched saturated C₁₋₄alkyl

### The Group —R^{Q1N}.

(234) A compound according to any one of (1) to (233), wherein:
   each —R^{Q1N}, if present, is independently:
   —R^{Q1NC},
   —RQ1NX.
   —R^{Q1Nhet}-L^{Q1N}-R^{Q1Nhet},
   -L^{Q1N}-OH, -L^{Q1N}-ORQ1NC
   -L^{Q1N}-C(═O)OH, -L^{Q1N}—C(═O)OR^{Q1NC},
   -L^{Q1N}-C(═O)NH₂, -L^{Q1N}-C(═O)NHRQ1NK, -L^{Q1N}-C(═O)NR^{Q1NC}₂, -L^{Q1N}-C(═O)R^{Q1NP},
   -L^{Q1N}-NH₂, -L^{Q1N}-NHR^{Q1NC}, -L^{Q1N}-NR^{Q1NC}₂, -L^{Q1N}-RQ1NM
   -L^{Q1N}—NHC(═O)OR^{Q1NC},
(235) A compound according to any one of (1) to (233), wherein:
   each —R^{Q1N}, if present, is independently:
   —R^{Q1NC},
   -L^{Q1N}—C(═O)NR^{Q1NC}₂, or -LOIN—C(═O)RQ1NP.
(236) A compound according to any one of (1) to (233), wherein:
   each —R^{Q1N}, if present, is independently:
   —R^{Q1Nhet} or -L^{Q1N}-RQ1Nhet
(237) A compound according to any one of (1) to (233), wherein:
   each —R^{Q1N}, if present, is:
   —R^{Q1Nhet},
(238) A compound according to any one of (1) to (233), wherein:
   each —R^{Q1N}, if present, is:
   -L^{Q1N}—C(═O)NH₂, -L^{Q1N}-C(═O)NHRQ1NK, -L^{Q1N}-C(═O)NR^{Q1N} G2, or -L^{Q1N}-C(═O)RQ1NP.
(239) A compound according to any one of (1) to (233), wherein:
   each —R^{Q1N}, if present, is:
   -L^{Q1N}-C(═O)NHR^{Q1NK}, -L^{Q1N}-C(═O)NR^{Q1NG}₂, or -LOIN—C(═O)RQ1NP.
(240) A compound according to any one of (1) to (233), wherein:
   each —R^{Q1N}, if present, is:
   -L^{Q1N}-C(═O)NHRQ1NK
(241) A compound according to any one of (1) to (233), wherein:
   each —R^{Q1N}, if present, is:
   -L^{Q1N}-C(═O)RQ1NP.
(242) A compound according to any one of (1) to (233), wherein:
   each —R^{Q1N}, if present, is independently:
   —R^{Q1NC},
(243) A compound according to any one of (1) to (233), wherein:
   each —R^{Q1N}, if present, is independently:
   -L^{Q1N}-OH or -L^{Q1N}-ORQ1NC.
(244) A compound according to any one of (1) to (233), wherein:
   each —R^{Q1N}, if present, is independently:
   -L^{Q1N}-NH₂, -LQ1N—NHR^{Q1NC}, -L^{Q1N}-NR^{Q1NC}₂, or -L^{Q1N}-RQ1NM
(245) A compound according to any one of (1) to (233), wherein:
   each —R^{Q1N}, if present, is independently:
   —R^{Q1NC} -L^{Q1N}-R^{Q1NC}; -RQ1NX
   —R^{Q1Nhet}, -LQ1N—R^{Q1Nhet},
   -L^{Q1N}-OH, -L^{Q1N}-OR^{Q1NC},
   —S(═O)₂R^{Q1NC},
   -L^{Q1N}-C(═O)OH, -LOIN—C(═O)OR^{Q1NC},
   -L^{Q1N}-C(═O)NH₂, -LIN—C(═O)NHRQ1NK, -L^{Q1N}-C(═O)NR^{Q1NC}₂, -L^{Q1N}-C(═O)R^{Q1NP},
   -L^{Q1N}-NH₂, -L^{Q1N}-NR^{Q1NC}₂,
   or -L^{Q1N}—NHC(═O)OR^{Q1NC};
(246) A compound according to any one of (1) to (233), wherein:
   each —R^{Q1N}, if present, is independently:
   —RQ1NC
   —R^{Q1NX},
   —R^{Q1Nhet}, -LQ1N—R^{Q1Nhet},
   —S(═O)₂RQ1NC
   -L^{Q1N}-C(═O)NR^{Q1NG}₂, or -L^{Q1N}-C(═O)R^{Q1N} P.

### The Group -RQ1NC

(247) A compound according to any one of (1) to (246), wherein:
   each —R^{Q1NC} if present, is independently linear or branched saturated C₁₋₄alkyl, C₃₋₆cycloalkyl, C₃₋₆cycloalkyl-C₁₋₃alkyl, phenyl, phenyl-C₁₋₃alkyl, wherein each phenyl is optionally substituted with one or more groups selected from: —F, —Cl, —CH₃, —CF₃, —OH, —OCH₃, —NH₂, —NH(CH₃), and —N(CH₃)₂.
(248) A compound according to any one of (1) to (246), wherein:
   each —R^{Q1NC} if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(249) A compound according to any one of (1) to (246), wherein:
   each —R^{Q1NC} if present, is independently linear or branched saturated C₁₋₄alkyl, phenyl, or benzyl.
(250) A compound according to any one of (1) to (246), wherein:
   each —R^{Q1NC} if present, is independently linear or branched saturated C₁₋₄alkyl.
(251) A compound according to any one of (1) to (246), wherein:
   each —R^{Q1NC} if present, is independently -Me, -Et, -nPr, -iPr, -nBu, or -tBu.
(252) A compound according to any one of (1) to (246), wherein:
   each —R^{Q1NC} if present, is independently -Me, -Et, -nPr, or -iPr.
(253) A compound according to any one of (1) to (246), wherein:
   each —R^{Q1NC} if present, is independently -Me or -Et.
(254) A compound according to any one of (1) to (246), wherein:
   each —R^{Q1NC} if present, is -Me.
(255) A compound according to any one of (1) to (246), wherein:
   each —R^{Q1NC} if present, is independently linear or branched saturated C₁₋₄alkyl, C₃₋₆cycloalkyl, phenyl, or C₅₋₆heteroaryl, wherein each C₁₋₄alkyl is optionally substituted by —F, —OH, -CEN, —SO₂—CH₃, or —OCH₃, wherein each cycloalkyl, phenyl and heteroaryl is optionally substituted with one or more groups selected from: —F, —Cl, —Br, linear or branched saturated C₁₋₄alkyl, —CHF₂, —CF₃, —CH₂—O—CH₃, —OCH₂CH₃, and —C(═O)—NH—phenyl, wherein C₁₋₄alkyl and phenyl are independently optionally substituted by —CH₃ or —OH.
(256) A compound according to any one of (1) to (246), wherein:
   each —R^{Q1NC} if present, is independently methyl, ethyl, propyl, i-propyl, butyl, i-butyl, t-butyl, cyclopropyl, cyclobutyl, cyclopentyl, cyclohexyl, phenyl, pyrimidinyl, oxazolyl, pyridyl, thiazolyl, imidazolyl, or pyrazolyl, wherein each methyl, ethyl, propyl, i-propyl, butyl, i-butyl, or t-butyl is optionally substituted by —F, —OH, —C≡N, —SO₂—CH₃, or —OCH₃, wherein each cyclopropyl, cyclobutyl, cyclopentyl, cyclohexyl, phenyl, pyrimidinyl, oxazolyl, pyridyl, thiazolyl, imidazolyl, or pyrazolyl, is optionally substituted with one or more groups selected from: —F, —Cl, —Br, linear or branched saturated
   C₁₋₄alkyl, —CHF₂, —CF₃, —CH₂—O—CH₃, —OCH₂CH₃, and —C(═O)—NH—phenyl, wherein C₁₋₄alkyl and phenyl are independently optionally substituted by —CH₃ or —OH.
(257) A compound according to any one of (1) to (246), wherein:
   each —ROINC, if present, is independently linear or branched saturated C₁₋₄alkyl, C₃₋₆cycloalkyl, phenyl, or C₅₋₆heteroaryl, wherein each C₁₋₄alkyl is optionally substituted by—F, —OH, —C≡N, —SO₂—CH₃, or —OCH₃, wherein each cycloalkyl, phenyl and heteroaryl is optionally substituted with one or more groups selected from: —F, —Cl, —CF₃, —OCH₃.

### The Group -RQ1NX

(258) A compound according to any one of (1) to (257), wherein:
   each —R^{Q1N} X, if present, is independently linear or branched saturated C₁₋₄fluoroalkyl.
(259) A compound according to any one of (1) to (257), wherein:
   each —R^{Q1N} X, if present, is independently —CF₃, —CHF₂, —CH₂CF₃, or —CH₂CHF₂.
(260) A compound according to any one of (1) to (257), wherein:
   each —R^{Q1N} X, if present, is —CHF₂.
(261) A compound according to any one of (1) to (257), wherein:
   each —R^{Q1N} X, if present, is —CH₂CH₂F, —CH₂CHF₂, —CH₂CF₃, and —CH₂CH₂Br.

### The Group -L^{Q1N}-

(262) A compound according to any one of (1) to (261), wherein:
   each -L^{Q1N}-, if present, is independently —CH₂—, —C(CH₃)₂—, —CH₂CH₂—, or —CH₂CH₂CH₂—.
(263) A compound according to any one of (1) to (261), wherein:
   each -L^{Q1N}-, if present, is independently —CH₂—, —CH₂CH₂—, or —CH₂CH₂CH₂—.
(264) A compound according to any one of (1) to (226112), wherein:
   each -L^{Q1N}-, if present, is —CH₂—.
(265) A compound according to any one of (1) to (261), wherein:
   each -L^{Q1N}-, if present, is —CH₂CH₂—.
(266) A compound according to any one of (1) to (261), wherein:
   each -L^{Q1N}-, if present, is —CH₂CH₂CH₂—.
(267) A compound according to any one of (1) to (261), wherein:
   each -L^{Q1N}—, if present, is —CH₂—, —C(CH₃) 2CH₂—-, —CH₂CH₂—, or —CH₂CH₂CH₂—, wherein-CH₂—, —C(CH₃) 2CH₂—-, —CH₂CH₂—, or —CH₂CH₂CH₂—is optionally substituted by —OH or —OMe.

### The Group -RQ1NM

(268) A compound according to any one of (1) to (267), wherein:
   each -RO1NM, if present, is independently azetidino, pyrrolidino, piperidino, piperazino, morpholino, thiomorpholino, azepano, or diazepano, and is:
   optionally substituted on carbon with one or more groups —R^{Q1NMM}, and
   optionally substituted on secondary nitrogen, if present, with a group selected from: —R^{Q1NMM}, —C(═O)R^{Q1NMM}, —C(═O)OR^{Q1NMM}, —C(═O)NH₂, —C(═O)NHR^{Q1NMM}, —C(═O)NR^{Q1NMM}₂, and —S(═O)₂RQ1NMM
(269) A compound according to any one of (1) to (267), wherein:
   each —R^{Q1N} M, if present, is independently pyrrolidino, piperidino, piperazino, or morpholino, and is:
   optionally substituted on carbon with one or more groups —R^{Q1NMM}, and
   optionally substituted on secondary nitrogen, if present, with a group selected from: —R^{Q1NMM}, —C(═O)R^{Q1NMM}, —C(═O)OR^{Q1NMM}, —C(═O)NH₂, —C(═O)NHR^{Q1NMM}, —C(═O)NR^{Q1NMM}₂, and —S(═O)₂RQ1NMM
(270) A compound according to any one of (1) to (267), wherein:
   each —R^{Q1N} M, if present, is independently pyrrolidino, piperidino, piperazino, or morpholino, and is:
   optionally substituted on carbon with one or more groups —R^{Q1NMM}; and
   optionally substituted on secondary nitrogen, if present, with a group selected from: —R^{Q1NMM}, —C(═O)R^{Q1N} MM, and —C(═O)ORQ1NMM

### The Group -RQ1NMM

(271) A compound according to any one of (1) to (270), wherein:
   each —R^{Q1N} MM, if present, is independently linear or branched saturated C₁₋₄alkyl, C₃₋₆cycloalkyl, C₃₋₆cycloalkyl-C₁₋₃alkyl, phenyl, phenyl-C₁₋₃alkyl, wherein each phenyl is optionally substituted with one or more groups selected from: —F, —Cl, —CH₃, —CF₃, —OH, —OCH₃, —NH₂, —NH(CH₃), and —N(CH₃)₂.
(272) A compound according to any one of (1) to (270), wherein:
   each —R^{Q1N} MM, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(273) A compound according to any one of (1) to (270), wherein:
   each —R^{Q1N} MM, if present, is independently linear or branched saturated C₁₋₄alkyl, phenyl, or benzyl.
(274) A compound according to any one of (1) to (270), wherein:
   each —R^{Q1NMM}, if present, is independently linear or branched saturated C₁₋₄alkyl.
(275) A compound according to any one of (1) to (270), wherein:
   each —R^{Q1NMM}, if present, is -Me.

### The Group —R^{Q1Nhet},

(276) A compound according to any one of (1) to (275), wherein:
   each —R^{Q1Nhet}, if present, is independently azetidinyl, oxetanyl, pyrrolidinyl, tetrahydrofuranyl, piperidinyl, piperazinyl, tetrahydropyranyl, morpholinyl, thiomorpholinyl, azepanyl, or diazepanyl;
   and is:
   optionally substituted on sulfur, if present, with one or two groups ═O;
   optionally substituted on carbon with one or more groups —R^{Q1NHH} or ═O; and
   optionally substituted on secondary nitrogen, if present, with a group selected from: —R^{Q1NHH}, —C(═O)RQ1NJ, —C(═O)OR^{Q1NHH}, —C(═O)NH₂, —C(═O)NHR^{Q1NHH}, —C(═O)NR^{Q1NHH}₂, and —S(═O)₂RQ1NHH
(277) A compound according to any one of (1) to (275), wherein:
   each —R^{Q1Nhet}, if present, is independently oxetanyl, pyrrolidinyl, tetrahydrofuranyl, piperidinyl, piperazinyl, tetrahydropyranyl, or morpholinyl;
   and is:
   optionally substituted on carbon with one or more groups —R^{Q1NHH} or ═O; and
   optionally substituted on secondary nitrogen, if present, with a group selected from: —R^{Q1NHH}, —C(═O)R^{Q1NJJ}, —C(═O)OR^{Q1NHH}, —C(═O)NH₂, —C(═O)NHR^{Q1NHH}, —C(═O)NR^{Q1NHH}₂, and —S(═O)₂RQ1NHH
(278) A compound according to any one of (1) to (275), wherein:
   each —R^{Q1Nhet}, if present, is independently pyrrolidinyl, piperidinyl, or piperazinyl; and is:
   optionally substituted on carbon with one or more groups —R^{Q1NHH} or ═O; and
   optionally substituted on secondary nitrogen, if present, with a group selected from: —R^{Q1NHH}, —C(═O)R^{Q1NHH}, —C(═O)OR^{Q1NHH}, —C(═O)NH₂, —C(═O)NHR^{Q1NHH},1 —C(═O)NR^{Q1NHH}₂, and —S(═O)₂RQ1NHH
(279) A compound according to any one of (1) to (275), wherein:
   each —R^{Q1Nhet}, if present, is independently pyrrolidinyl or piperidinyl;
   and is:
   optionally substituted on carbon with one or more groups —R^{Q1NHH} or ═O; and
   optionally substituted on secondary nitrogen, if present, with a group selected from: —R^{Q1NHH}, —C(═O)R^{Q1NJJ}, —C(═O)OR^{Q1NHH}, —C(═O)NH₂, —C(═O)NHR^{Q1NHH}, —C(═O)NR^{Q1NHH}₂, and —S(═O)₂RQ1NHH
(280) A compound according to any one of (1) to (275), wherein:
   each —R^{Q1Nhet}, if present, is pyrrolidinyl;
   and is:
   optionally substituted on carbon with one or more groups —R^{Q1NHH} or ═O; and optionally substituted on secondary nitrogen, if present, with a group selected from: —R^{Q1NHH}, —C(═O)R^{Q1NJJ}, —C(═O)OR^{Q1NHH}, —C(═O)NH₂, —C(═O)NHR^{Q1NHH}, —C(═O)NR^{Q1NHH}₂, and —S(═O)₂RQ1NHH
(281) A compound according to any one of (1) to (275), wherein:
   each —R^{Q1Nhet}, if present, is independently non-aromatic C₃₋₇heterocyclyl;
   and is:
   optionally substituted on carbon with one or more groups ═O; and
   optionally substituted on secondary nitrogen, if present, with a group selected from: —R^{Q1NHH}, —C(═O)R^{Q1NJJ}, —C(═O)OR^{Q1NHH}, —C(═O)NHR^{Q1NHH}, and —C(═O)NRQ1NHH2.
(282) A compound according to any one of (1) to (275), wherein:
   each —R^{Q1Nhet}, if present, is independently oxetanyl, piperidinyl, pyrrolidinyl, tetrahydrofuranyl, tetrahydropyranyl, morpholinyl, azetidinyl, or tetrahydrothiophenyl;
   and is:
   optionally substituted on carbon with one or more groups ═O; and
   optionally substituted on secondary nitrogen, if present, with a group selected from: —R^{Q1NHH}, —C(═O)R^{Q1NJJ}, —C(═O)OR^{Q1NHH}, —C(═O)NHR^{Q1NHH}, and —C(═O)NRQ1NHH2.

### The Group -RQ1NHH

(283) A compound according to any one of (1) to (283), wherein:
   each —R^{Q1NHH}, if present, is independently linear or branched saturated C₁₋₄alkyl,
   C₃₋₆cycloalkyl, C₃₋₆cycloalkyl-C₁₋₃alkyl, phenyl, phenyl-C₁₋₃alkyl, wherein each phenyl is optionally substituted with one or more groups selected from: —F, —Cl, —CH₃, —CF₃, —OH, —OCH₃, —NH₂, —NH(CH₃), and —N(CH₃)₂.
(284) A compound according to any one of (1) to (283), wherein:
   each —R^{Q1NHH}, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(285) A compound according to any one of (1) to (283), wherein:
   each —R^{Q1NHH}, if present, is independently linear or branched saturated C₁₋₄alkyl, phenyl, or benzyl.
(286) A compound according to any one of (1) to (283), wherein:
   each —R^{Q1NHH}, if present, is independently linear or branched saturated C₁₋₄alkyl.
(287) A compound according to any one of (1) to (283), wherein:
   each —R^{Q1NHH}, if present, is independently -Me, -Et, -nPr, -iPr, -nBu, or -tBu.
(288) A compound according to any one of (1) to (283), wherein:
   each —R^{Q1NHH}, if present, is independently -Me, -Et, -nPr, or -iPr.
(289) A compound according to any one of (1) to (283), wherein:
   each —R^{Q1NHH}, if present, is independently -Me or -Et.
(290) A compound according to any one of (1) to (283), wherein:
   each —R^{Q1NHH}, if present, is -Me.
(291) A compound according to any one of (1) to (283), wherein:
   each —R^{Q1NHH}, if present, is independently linear or branched saturated C₁₋₄alkyl, C₃₋₆cycloalkyl, or phenyl-C₁₋₃alkyl.
(292) A compound according to any one of (1) to (283), wherein:
   each —R^{Q1NHH}, if present, is independently -Me, -Et, -tBu, cyclopropyl, benzyl or phenylethyl.

### The Group -RQ1NJJ

(293) A compound according to any one of (1) to (292), wherein:
   —R^{Q1NHH}, if present, is —R^{J1}, -L^{J}-R^{J2}, —R^{J3}, -L^{J}-R^{J3}, —R^{J4}, -L^{J}-R^{J4}, or -L^{J}-RJ5
(294) A compound according to any one of (1) to (292), wherein:
   —R^{Q1NHH}, if present, is —R^{J1}, -L^{J}-R^{J2}, -L^{J}-R^{J3}, -L^{J}-R^{J4}, or -L^{J}-RJ5
(295) A compound according to any one of (1) to (292), wherein:
   —Ra1NJJ, if present, is —R^{J1}, -L^{J}-R^{J3}, -L^{J}-R^{J4}, or -L^{J}-RJ5.
(296) A compound according to any one of (1) to (292), wherein:
   —R^{Q1NHH}, if present, is —R^{J1}, -L^{J}-R^{J4}, or -L^{J}-RJ5.
(297) A compound according to any one of (1) to (292), wherein:
   —RQ1NJJ, if present, is —R^{J1} or -L^{J}-RJ4
(298) A compound according to any one of (1) to (292), wherein:
   —R^{Q1NHH}, if present, is -L^{J}-R^{J2}, -L^{J}-R^{J3}, -L^{J}-R^{J4}, or -L′-RJ5.
(299) A compound according to any one of (1) to (292), wherein:
   —RQ1NJJ, if present, is -L^{J}-R^{J3}, -L^{J}-R^{J4}, or -L^{J}-RJ5
(300) A compound according to any one of (1) to (292), wherein:
   —R^{Q1NHH}, if present, is -L^{J}-R^{J4} or -L^{J}-RJ5.
(301) A compound according to any one of (1) to (292), wherein:
   —RQ1NJJ, if present, is -L^{J}-RJ4.

### The Group -RJ1

(302) A compound according to any one of (1) to (301), wherein:
   —R^{J1}, if present, is linear or branched saturated C1-6alkyl; and is optionally substituted with one or more groups selected from: —F, —OH, —OR^{JJ}, —O-phenyl, —C(═O)OH, —C(═O)OR^{J}, —NH₂, —NHR^{J}, and -NRJ2.
(303) A compound according to any one of (1) to (301), wherein:
   —R^{J1}, if present, is linear or branched saturated C₁₋₄alkyl; and is optionally substituted with one or more groups selected from: —F, —OH, —OR^{J}, —O-phenyl, —C(═O)OH, —C(═O)OR^{JJ}, —NH₂, —NHR^{JJ}, and -NRJ2.
(304) A compound according to any one of (1) to (301), wherein:
   R^{J1}, if present, is linear or branched saturated C₁₋₄alkyl; and is optionally substituted with one or more groups selected from: —F, —OH, or -ORJ.
(305) A compound according to any one of (1) to (301), wherein:
   —R^{J1}, if present, is linear or branched saturated C₁₋₄alkyl.
(306) A compound according to any one of (1) to (301), wherein:
   —R^{J1}, if present, is -Me, -Et, -nPr, -iPr, -nBu, or -tBu.
(307) A compound according to any one of (1) to (301), wherein:
   —R^{J1}, if present, is -Me, -Et, -nPr, or -iPr.
(308) A compound according to any one of (1) to (301), wherein:
   —R^{J1}, if present, is -Me or -Et.
(309) A compound according to any one of (1) to (301), wherein:
   —R^{J1}, if present, is -Me.
(310) A compound according to any one of (1) to (301), wherein:
   —R^{J1}, if present, is linear or branched saturated C1-6alkyl; and is optionally substituted with one or more groups selected from: —F, —OR^{J}, —O-phenyl, and —C(═O)ORJ.
(311) A compound according to any one of (1) to (301), wherein:
   —R^{J1}, if present, is -Me, -Et, -iPr, -iBu; and is optionally substituted with one or more groups selected from: —F, —OR^{JJ}, —O-phenyl, and —C(═O)ORJJ The Group-RJ2
(312) A compound according to any one of (1) to (311), wherein:
   each —R^{J2}, if present, is independently cyclopropyl, cyclobutyl, cyclopentyl, or cyclohexyl.
(313) A compound according to any one of (1) to (311), wherein:
   each —R^{J2}, if present, is cyclopropyl.
(314) A compound according to any one of (1) to (311), wherein:
   each —R^{J2}, if present, is independently cyclobutyl.
(315) A compound according to any one of (1) to (311), wherein:
   each —R^{J2}, if present, is independently cyclopentyl.
(316) A compound according to any one of (1) to (311), wherein:
   each —R^{J2}, if present, is independently cyclohexyl.

### The Group -RJ3

(317) A compound according to any one of (1) to (316), wherein:
   each —R^{J3}, if present, is independently non-aromatic C₃₋₇heterocyclyl.
(318) A compound according to any one of (1) to (316), wherein:
   each —R^{J3}, if present, is independently azetidinyl, oxetanyl, pyrrolidinyl, tetrahydrofuranyl, piperidinyl, piperazinyl, tetrahydropyranyl, morpholinyl, thiomorpholinyl, azepanyl, or diazepanyl;
   and is:
   optionally substituted on sulfur, if present, with one or two groups ═O;
   optionally substituted on carbon with one or more groups selected from —F, —R^{J}, —CF₃, —OH, —OR^{J}, —NH₂, —NHR^{J}, and -NRJ2; and
   optionally substituted on secondary nitrogen, if present, with a group selected from: —R^{J}, —C(═O)R^{JJ}, —C(═O)OR^{J}, and —S(═O)₂RJJ.
(319) A compound according to any one of (1) to (316), wherein:
   each —R^{J3}, if present, is independently pyrrolidinyl, tetrahydrofuranyl, piperidinyl, piperazinyl, tetrahydropyranyl, or morpholinyl;
   and is:
   optionally substituted on carbon with one or more groups selected from —F, —R^{J}, —CF₃, —OH, —OR^{JJ}, —NH₂, —NHR^{J}, and —NR^{JJ}₂; and optionally substituted on secondary nitrogen, if present, with a group selected from: —R^{JJ}, —C(═O)R^{J}, —C(═O)OR^{JJ}, and —S(═O)₂RJJ.
(320) A compound according to any one of (1) to (316), wherein:
   each —R^{J3}, if present, is independently pyrrolidinyl, tetrahydrofuranyl, piperidinyl, piperazinyl, tetrahydropyranyl, or morpholinyl;
   and is:
   optionally substituted on carbon with one or more groups selected from —F and -RJ; and
   optionally substituted on secondary nitrogen, if present, with a group selected from: —R^{J}, —C(═O)R″, —C(═O)OR^{JJ}, and —S(═O)₂RJJ.
(321) A compound according to any one of (1) to (316), wherein:
   each —R^{J3}, if present, is independently pyrrolidinyl, tetrahydrofuranyl, piperidinyl, piperazinyl, tetrahydropyranyl, or morpholinyl;
   and is:
   optionally substituted on secondary nitrogen, if present, with a group selected from: —R^{J}, —C(═O)R^{J}, —C(═O)OR^{JJ}, and —S(═O)₂RJJ.
(322) A compound according to any one of (1) to (316), wherein:
   each —R^{J3}, if present, is independently tetrahydropyranyl or piperidinyl.

### The Group -RJ4

(323) A compound according to any one of (1) to (322), wherein:
   each —R^{J4}, if present, is phenyl; and is optionally substituted with one or more groups selected from: —F, —Cl, —R^{J}, —OH, —NH₂, —NHR^{J}, and -NRJJ.
(324) A compound according to any one of (1) to (322), wherein:
   each —R^{J4}, if present, is phenyl.
(325) A compound according to any one of (1) to (322), wherein:
   each —R^{J4}, if present, is phenyl; and is optionally substituted with one or more groups selected from: —F, —OR^{JJ}, —NH₂, and -NRJJ2.

### The Group -RJ5

(326) A compound according to any one of (1) to (325), wherein:
   each —R^{J5}, if present, is independently C₅₋₆heteroaryl.
(327) A compound according to any one of (1) to (325), wherein:
   each —R^{J5}, if present, is independently pyrrolyl, furanyl, thienyl, pyrazolyl, oxazolyl, isoxazolyl, thiazolyl, isothiazolyl, pyridinyl, pyrimidinyl, or pyridizinyl; and is: optionally substituted on carbon with one or more groups selected from —F, —R^{J}, —CF₃, —OH, —OR″, —NH₂, —NHR^{J}, and -NRJ2; and optionally substituted on secondary nitrogen, if present, with a group selected from: —R^{JJ}, —C(═O)R^{JJ}, —C(═O)OR^{JJ}, and —S(═O)₂RJJ.
(328) A compound according to any one of (1) to (325), wherein:
   each —R^{J5}, if present, is independently pyrrolyl, furanyl, thienyl, pyrazolyl, pyridinyl, pyrimidinyl, or pyridizinyl; and is:
   optionally substituted on carbon with one or more groups selected from —F, —R^{J}, —CF₃, —OH, —OR^{JJ}, —NH₂, —NHR^{J}, and —NR^{JJ}₂; and
   optionally substituted on secondary nitrogen, if present, with a group selected from: —R^{J}, —C(═O)R^{JJ}, —C(═O)OR^{JJ}, and —S(═O)₂RJJ.
(329) A compound according to any one of (1) to (325), wherein:
   each —R^{J5}, if present, is independently thienyl, pyrazolyl, or pyridinyl; and is:
   optionally substituted on carbon with one or more groups selected from —F, —R^{J}, —CF₃, —OH, —OR^{JJ}, —NH₂, —NHR^{J}, and —NR^{JJ}₂; and
   optionally substituted on secondary nitrogen, if present, with a group selected from: —R^{J}, —C(═O)R^{JJ}, —C(═O)OR^{JJ}, and —S(═O)₂RJJ.
(330) A compound according to any one of (1) to (325), wherein:
   each —R^{J5}, if present, is independently thienyl, pyrazolyl, or pyridinyl.

### The Group -L^{J}-

(331) A compound according to any one of (1) to (330), wherein:
   each -L^{J}-, if present, is independently —CH₂—, —CF₂—, —C(CH₃)₂—, —CH₂CH₂—, or —CH₂CH₂CH₂—.
(332) A compound according to any one of (1) to (330), wherein:
   each -L^{J}-, if present, is independently linear or branched saturated C₁₋₄alkylene.
(333) A compound according to any one of (1) to (330), wherein:
   each -L^{J}-, if present, is independently —CH₂—, —C(CH₃)₂—, —CH₂CH₂—, or —CH₂CH₂CH₂—.
(334) A compound according to any one of (1) to (330), wherein:
   each -L^{J}-, if present, is independently —CH₂—, —CH₂CH₂—, or —CH₂CH₂CH₂—.
(335) A compound according to any one of (1) to (330), wherein:
   each -L′-, if present, is —CH₂—.
(336) A compound according to any one of (1) to (330), wherein:
   each -L^{J}-, if present, is —CH₂CH₂—.
(337) A compound according to any one of (1) to (330), wherein:
   each -L^{J}-, if present, is —CH₂CH₂CH₂—.
(338) A compound according to any one of (1) to (330), wherein:
   each -L^{J}-, if present, is independently —CH₂—, or —CH₂CH₂—, and is optionally substituted with one or more-F.

### The Group -RJJ

(339) A compound according to any one of (1) to (338), wherein:
   each —R^{J}, if present, is:
   -Me, -Et, -nPr, -iPr, -nBu, or -tBu.
-(340) A compound according to any one of (1) to (338), wherein:
   each —R^{J}, if present, is:
   -Me, -Et, -nPr, or -iPr.
(341) A compound according to any one of (1) to (338), wherein:
   each —R^{JJ}, if present, is:
   -Me or -Et.
(342) A compound according to any one of (1) to (338), wherein:
   each —R^{JJ}, if present, is:
   -Me.

### The Group —R^{Q1NK}

(343) A compound according to any one of (1) to (342), wherein:
—R^{Q1NK}, if present, is —R^{K1}, —R^{K2}, -L^{K}-R^{K2}, —R^{K3}, or -L^{K}-R^{K3};
(344) A compound according to any one of (1) to (342), wherein:
—R^{Q1NK}, if present, is —R^{K1}, —R^{K2}, —R^{K3}, or -L^{K}-R^{K3},
(345) A compound according to any one of (1) to (342), wherein:
—R^{Q1NK}, if present, is —R^{K1}.
(346) A compound according to any one of (1) to (342), wherein:
—R^{Q1NK}, if present, is —R^{K2}.
(347) A compound according to any one of (1) to (342), wherein:
—R^{Q1NK}, if present, is —R^{K3}.
(348) A compound according to any one of (1) to (342), wherein:
—R^{Q1NK}, if present, is -L^{K}-R^{K3}.

### The Group —R^{K1}

(349) A compound according to any one of (1) to (348), wherein:
   —R^{K1}, if present, is linear or branched saturated C1-6alkyl; and is optionally substituted with one or more groups selected from: —F, —OH, —OR^{KK}, —O-phenyl, —C(═O)OH, —C(═O)OR^{KK}, —NH₂, —NHR^{KK}, and —NR^{KK}₂.
(350) A compound according to any one of (1) to (348), wherein:
   —R^{K1}, if present, is linear or branched saturated C₁₋₄alkyl; and is optionally substituted with one or more groups selected from: —F, —OH, —OR^{KK}, —O-phenyl, —C(═O)OH, —C(═O)OR^{KK}, —NH₂, —NHR^{KK}, and —NR^{KK}₂.
(351) A compound according to any one of (1) to (348), wherein:
   —R^{K1}, if present, is linear or branched saturated C₁₋₄alkyl; and is optionally substituted with one or more groups selected from: —F, —OH, or —OR^{KK}.
(352) A compound according to any one of (1) to (348), wherein:
   R^{K1}, if present, is linear or branched saturated C₁₋₄alkyl.
(353) A compound according to any one of (1) to (348), wherein:
   —R^{K1}, if present, is -Me, -Et, -nPr, -iPr, -nBu, or -tBu.
(354) A compound according to any one of (1) to (348), wherein:
   —R^{K1}, if present, is -Me, -Et, -nPr, or -iPr.
(355) A compound according to any one of (1) to (348), wherein:
   —R^{K1}, if present, is -Me or -Et.
(356) A compound according to any one of (1) to (348), wherein:
   —R^{K1}, if present, is -Me.
(357) A compound according to any one of (1) to (348), wherein:
   —R^{K1}, if present, is linear or branched saturated C1-7alkyl; and is optionally substituted with one or more groups selected from: —OH, —OR^{KK}, —OCH₂CH₂OCH₃, and —NR^{KK}₂.
(358) A compound according to any one of (1) to (348), wherein:
   —R^{K1}, if present, is -Me, -Et, -nPr, -iPr, -tBu, or heptane; and is optionally substituted with one or more groups selected from: —OH, —OR^{KK}, —OCH₂CH₂OCH₃, and —NR^{KK}₂.

### The Group —R^{K2}

(359) A compound according to any one of (1) to (358), wherein:
   each —R^{K2}, if present, is independently C₃₋₆cycloalkyl.
(360) A compound according to any one of (1) to (358), wherein:
   each —R^{K2}, if present, is independently cyclopropyl, cyclobutyl, cyclopentyl, or cyclohexyl.
(361) A compound according to any one of (1) to (358), wherein:
   each —R^{K2}, if present, is cyclopropyl.
(362) A compound according to any one of (1) to (358), wherein:
   each —R^{K2}, if present, is independently cyclobutyl.
(363) A compound according to any one of (1) to (358), wherein:
   each —R^{K2}, if present, is independently cyclopentyl.
(364) A compound according to any one of (1) to (358), wherein:
   each —R^{K2}, if present, is independently cyclohexyl.

### The Group —R^{K3}

(365) A compound according to any one of (1) to (364), wherein:
each —R^{K3}, if present, is independently non-aromatic C₃₋₇heterocyclyl; and is:
   optionally substituted on secondary nitrogen, if present, with a group selected from: —R^{KK}, and —C(═O)OR^{KK}.
(366) A compound according to any one of (1) to (364), wherein:
   each —R^{K3}, if present, is independently morpholinyl, piperidinyl, piperazinyl, pyrrolidinyl, or tetrahydropyranyl; and is:
   optionally substituted on secondary nitrogen, if present, with a group selected from: —R^{KK}, and —C(═O)OR^{KK}.
(367) A compound according to any one of (1) to (364), wherein:
   each —R^{K3}, if present, is independently azetidinyl, oxetanyl, pyrrolidinyl, tetrahydrofuranyl, piperidinyl, piperazinyl, tetrahydropyranyl, morpholinyl, thiomorpholinyl, azepanyl, or diazepanyl;
   and is:

optionally substituted on sulfur, if present, with one or two groups ═O;
optionally substituted on carbon with one or more groups selected from —F, —R^{KK}, —CF₃, —OH, —OR^{KK}, —NH₂, —NHR^{KK}, and —NR^{KK2}; and
   optionally substituted on secondary nitrogen, if present, with a group selected from: —R^{KK}, —C(═O)R^{KK}, —C(═O)OR^{KK}, and —S(═O)₂R^{KK}.
(368) A compound according to any one of (1) to (364), wherein:
   each —R^{K3}, if present, is independently pyrrolidinyl, tetrahydrofuranyl, piperidinyl, piperazinyl, tetrahydropyranyl, or morpholinyl;
   and is:
   optionally substituted on carbon with one or more groups selected
from —F, —R^{KK}, —CF₃, —OH, —OR^{KK}, —NH₂, —NHR^{KK}, and —NR^{KK2}; and
   optionally substituted on secondary nitrogen, if present, with a group selected from: —R^{KK}, —C(═O)R^{KK}, —C(═O)OR^{KK}, and —S(═O)₂R^{KK}.
(369) A compound according to any one of (1) to (364), wherein:
   each —R^{K3}, if present, is independently pyrrolidinyl, tetrahydrofuranyl, piperidinyl, piperazinyl, tetrahydropyranyl, or morpholinyl;
   and is:
   optionally substituted on carbon with one or more groups selected from —F and —R^{KK}; and
   optionally substituted on secondary nitrogen, if present, with a group selected from: —R^{KK}, —C(═O)R^{KK}, —C(═O)OR^{KK}, and —S(═O)₂R^{KK}
(370) A compound according to any one of (1) to (364), wherein:
   each —R^{K3}, if present, is independently pyrrolidinyl, tetrahydrofuranyl, piperidinyl, piperazinyl, tetrahydropyranyl, or morpholinyl;
   and is:
   optionally substituted on secondary nitrogen, if present, with a group selected from: —R^{KK}, —C(═O)R^{KK}, —C(═O)OR^{KK}, and —S(═O)₂R^{KK}

### The Group —R^{K4}

(371) A compound according to any one of (1) to (370), wherein:
   each —R^{K4}, if present, is phenyl; and is optionally substituted with one or more groups selected from: —F, —Cl, —R^{KK}, —CF₃, —OH, and —OR^{KK}
(372) A compound according to any one of (1) to (370), wherein:
   each —R^{K4}, if present, is phenyl.

### The Group —R^{K5}

(373) A compound according to any one of (1) to (372), wherein:
   each —R^{K5}, if present, is independently pyrrolyl, furanyl, thienyl, pyrazolyl, oxazolyl, isoxazolyl, thiazolyl, isothiazolyl, pyridinyl, pyrimidinyl, or pyridizinyl; and is:
   optionally substituted on carbon with one or more groups selected from —F, —R^{KK}, —CF₃, —OH, —OR^{KK}, —NH₂, —NHR^{KK}, and —NR^{KK2}; and
   optionally substituted on secondary nitrogen, if present, with a group selected from: —R^{KK}, —C(═O)R^{KK}, —C(═O)OR^{KK}, and —S(═O)₂R^{KK}
(374) A compound according to any one of (1) to (372), wherein:
   each —R^{K5}, if present, is independently pyrrolyl, furanyl, thienyl, pyrazolyl, pyridinyl, pyrimidinyl, or pyridizinyl; and is:
   optionally substituted on carbon with one or more groups selected from —F, —R^{KK}, —CF₃, —OH, —OR^{KK}, —NH₂, —NHR^{KK}, and —NR^{KK2}; and
   optionally substituted on secondary nitrogen, if present, with a group selected from: —R^{KK}, —C(═O)R^{KK}, —C(═O)OR^{KK}, and —S(═O)₂R^{KK}.
(375) A compound according to any one of (1) to (372), wherein:
   each —R^{K5}, if present, is independently pyrrolyl, furanyl, thienyl, pyrazolyl, pyridinyl, pyrimidinyl, or pyridizinyl.

### The Group -LK

(376) A compound according to any one of (1) to (375), wherein:
   each -L^{K}-, if present, is independently linear or branched saturated C₁₋₄alkylene.
(377) A compound according to any one of (1) to (375), wherein:
   each -L^{K}-, if present, is independently —CH₂—, —C(CH₃)₂—, —CH₂CH₂—, or —CH₂CH₂CH₂—.
(378) A compound according to any one of (1) to (375), wherein:
   each -L^{K}-, if present, is independently —CH₂—, —CH₂CH₂—, or —CH₂CH₂CH₂—.
(379) A compound according to any one of (1) to (375), wherein:
   each -L^{K}-, if present, is independently —CH₂—, or —CH₂CH₂—.
(380) A compound according to any one of (1) to (375), wherein:
   each -L^{K}-, if present, is —CH₂—.
(381) A compound according to any one of (1) to (375), wherein:
   each -L^{K}-, if present, is —CH₂CH₂—.
(382) A compound according to any one of (1) to (375), wherein:
   each -L^{K}-, if present, is —CH₂CH₂CH₂—.

### The Group —R^{KK}

(383) A compound according to any one of (1) to (382), wherein:
   each —R^{KK}, if present, is:
   -Me, -Et, -nPr, -iPr, -nBu, or -tBu.
(384) A compound according to any one of (1) to (382), wherein:
   each —R^{KK}, if present, is:
   -Me, -Et, -nPr, or -iPr.
(385) A compound according to any one of (1) to (382), wherein:
   each —R^{KK}, if present, is:
   -Me, or -tBu.
(386) A compound according to any one of (1) to (382), wherein:
   each —R^{KK}, if present, is:
   -Me or -Et.
(387) A compound according to any one of (1) to (382), wherein:
   each —R^{KK}, if present, is:
   -Me.

### The Group —R^{Q1NP}

(388) A compound according to any one of (1) to (387), wherein:
   —R^{Q1NP}, if present, is independently non-aromatic C₃₋₁₁heterocyclyl having at least one N ring atom, and is attached via that N ring atom; and is:
   optionally substituted on carbon with one or more groups selected from —R^{Q1NPP}, —F, —OH, —OR^{Q1NPP}, and ═O; and
   optionally substituted on secondary nitrogen, if present, with a group selected from: —R^{Q1NPP}, —R^{Q1NPPX}, —C(═O)R^{Q1NPP}, —C(═O)OR^{Q1NPP}, —C(═O)NR^{Q1NPP}₂, and —S(═O)₂R^{Q1NPP}.
(389) A compound according to any one of (1) to (387), wherein:
   —R^{Q1NP}, if present, is independently morpholinyl, piperazinyl, piperidinyl, pyrrolidinyl, 2,5-diazabicyclo[2.2.1]heptanyl, 2,6-diazaspiro[3.3]heptanyl, 2,8-diazaspiro[4.5]decanyl, 1,4-diazepanyl, azetidinyl, 3,8-diazabicyclo[3.2.1]octanyl, 3-azabicyclo[3.1.0]hexanyl, azetidinyl, 2-azaspiro[3.3]heptanyl, or 2,6-diazaspiro[3.3]heptanyl, attached via a N ring atom; and is: optionally substituted on carbon with one or more groups selected
   from —R^{Q1NPP}, —F, —OH, —OR^{Q1NPP}, and ═O; and
   optionally substituted on secondary nitrogen, if present, with a group selected
   from: —R^{Q1NPP}, —R^{Q1NPPX}, —C(═O)R^{Q1NPP}, —C(═O)OR^{Q1NPP}, —C(═O)NR^{Q1NPP}₂, and —S(═O)₂R^{Q1NPP}.
(390) A compound according to any one of (1) to (387), wherein:
   —R^{Q1NP}, if present, is independently optionally substituted on carbon with one or more groups selected from —R^{Q1N} PP; and
   optionally substituted on secondary nitrogen, if present, with a group selected from: —R^{Q1NPP}.
(391) A compound according to any one of (1) to (387), wherein:
   —R^{Q1NP}, if present, is independently:
   azetidinyl, pyrrolidinyl, piperidinyl, piperazinyl, morpholinyl, thiomorpholinyl, azepanyl, diazepanyl, 3-azabicyclo[3.1.0]hexanyl, 3,6-diazabicyclo[3.1.1]heptanyl, 3-azabicyclo[3.1.1]heptanyl, 2,5-diazabicyclo[2.2.1]heptanyl, 2-azabicyclo[2.2.1]heptanyl, 3,8-diazabicyclo[3.2.1]octanyl, 3-azabicyclo[3.2.1]octanyl, 2-azaspiro[3.3]heptanyl, 2,6-diazaspiro[3.3]heptanyl, 6-azaspiro[3.4]octanyl, 2-azaspiro[3.4]octanyl, 2,7-diazaspiro[3.4]octanyl, 7-azaspiro[3.5]nonanyl, 2-azaspiro[3.5]nonanyl, 2,7-diazaspiro[3.5]nonanyl, 2,7-diazaspiro[4.4]nonanyl, 2,8-diazaspiro[4.5]decanyl, or 3,9-diazaspiro[5.5]undecanyl;
   and is attached via an N ring atom;
   and is:
   optionally substituted on sulfur, if present, with one or two ═O groups;
   optionally substituted on carbon with one or more groups selected from —R^{Q1NPP}, —F, —OH, —OR^{Q1NPP}, and ═O; and
   optionally substituted on secondary nitrogen, if present, with a group selected from: —R^{Q1NPP}, —R^{Q1NPPX}, —C(═O)R^{Q1NPP}, —C(═O)OR^{Q1NPP}, —C(═O)NH₂, —C(═O)NHR^{Q1NPP}, —C(═O)NR^{Q1NPP}₂, and —S(═O)₂R^{Q1NPP}.

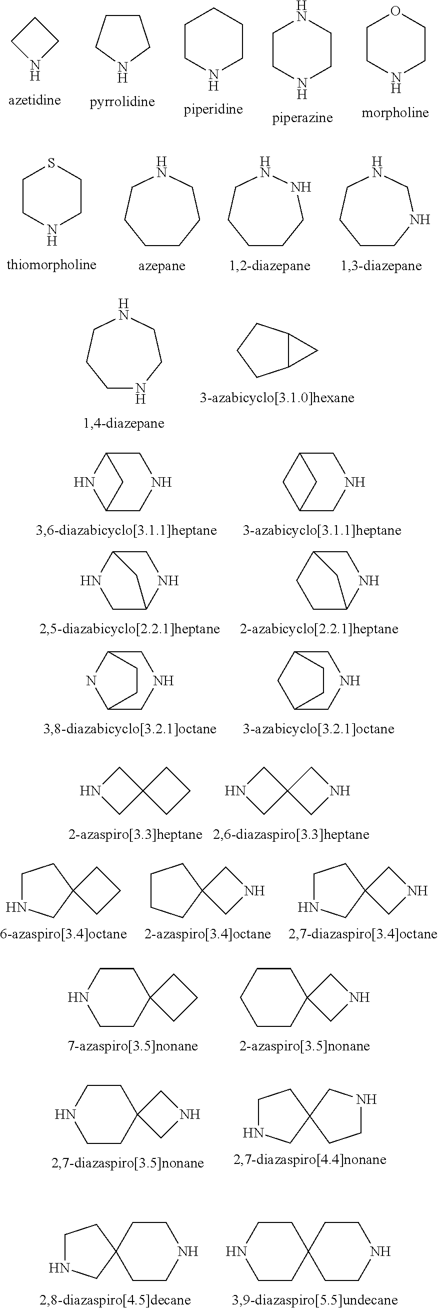

(392) A compound according to any one of (1) to (387), wherein:
   —R^{Q1NP}, if present, is independently:
   azetidinyl, pyrrolidinyl, piperidinyl, piperazinyl, 2-azaspiro[3.3]heptanyl, morpholinyl, thiomorpholinyl, azepanyl, or diazepanyl;
   and is attached via an N ring atom;
   and is:
   optionally substituted on sulfur, if present, with one or two ═O groups;
   optionally substituted on carbon with one or more groups selected from —R^{Q1NPP}, —F, —OH, —OR^{Q1NPP}, and ═O; and
   optionally substituted on secondary nitrogen, if present, with a group selected
   from: —R^{Q1NPP}, —R^{Q1NPPX}, —C(═O)R^{Q1NPP}, —C(═O)OR^{Q1NPP}, —C(═O)NH₂, —C(═O)NHR^{Q1NPP}, —C(═O)NR^{Q1NPP}₂, and —S(═O)₂R^{Q1NPP}.
(393) A compound according to any one of (1) to (387), wherein:
   —R^{Q1NP}, if present, is independently:
   azetidinyl, pyrrolidinyl, piperidinyl, piperazinyl, or morpholinyl;
   and is attached via an N ring atom;
   and is:
   optionally substituted on carbon with one or more groups selected from —R^{Q1NPP}, —F, —OH, —OR^{Q1NPP}, and ═O; and optionally substituted on secondary nitrogen, if present, with a group selected from: —R^{Q1NPP}, —R^{Q1NPPX}, —C(═O)R^{Q1NPP}, —C(═O)OR^{Q1NPP}, —C(═O)NH₂, —C(═O)NHR^{Q1NPP}, —C(═O)NR^{Q1NPP}₂, and —S(═O)₂R^{Q1NPP},

### The Group —R^{Q1NPP},

(394) A compound according to any one of (1) to (393), wherein:
   each —R^{Q1NPP}, if present, is independently linear or branched saturated C₁₋₄alkyl, C₃₋₆cycloalkyl, C₃₋₆cycloalkyl-C₁₋₃alkyl, or phenyl, wherein C₁₋₄alkyl is optionally substituted with —F, —OH or —OCH₃.
(395) A compound according to any one of (1) to (393), wherein:
   each —R^{Q1NPP}, if present, is independently -Me, -Et, -iPr, -iBu, -tBu, cyclopropyl, cyclobutyl, cyclopentyl, cyclohexyl, cyclopropylmethyl or phenyl, wherein-Me, -Et, -iPr, -iBu, or -tBu is optionally substituted with —F, —OH or —OCH₃.
(396) A compound according to any one of (1) to (393), wherein:
   each —R^{Q1NPP}, if present, is independently linear or branched saturated C₁₋₄alkyl, or phenyl, wherein C₁₋₄alkyl is optionally substituted with —F, —OH or —OCH₃.
(397) A compound according to any one of (1) to (393), wherein:
   each —R^{Q1NPP}, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(398) A compound according to any one of (1) to (393), wherein:
   each —R^{Q1NPP}, if present, is independently linear or branched saturated C₁₋₄alkyl, phenyl, or phenyl-CH₂—.
(399) A compound according to any one of (1) to (393), wherein:
   each —R^{Q1NPP}, if present, is independently linear or branched saturated C₁₋₄alkyl.
(400) A compound according to any one of (1) to (393), wherein:
   each —R^{Q1NPP}, if present, is:
   -Me, -Et, -nPr, -iPr, -nBu, or -tBu.
(401) A compound according to any one of (1) to (393), wherein:
   each —R^{Q1NPP}, if present, is:
   -Me, -Et, -nPr, or -iPr.
(402) A compound according to any one of (1) to (393), wherein:
   each —R^{Q1NPP}, if present, is:
   -Me or -Et.
(403) A compound according to any one of (1) to (393), wherein:
   each —R^{Q1NPP}, if present, is:
   -Me.

### The Group —R^{Q1NPPX}

(404) A compound according to any one of (1) to (403), wherein:
   —R^{Q1NPPX}, if present, is independently linear or branched saturated C₁₋₄fluoroalkyl.
(405) A compound according to any one of (1) to (403), wherein:
   —R^{Q1NPPX}, if present, is independently —CF₃, —CHF₂, —CH₂CF₃, or —CH₂CHF₂.
(406) A compound according to any one of (1) to (403), wherein:
   —R^{Q1NPPX}, if present, is —CH₂CF₃.

### The Group —R^{Q2C}

(407) A compound according to any one of (1) to (406), wherein:
   each —R^{Q2C}, if present, is independently:
   —F.
   —R^{Q2CC}, —R^{Q2CX},
   —OH, —OR^{Q2CC}, —OR^{Q2CX},
   —NH₂, —NHR^{Q2CC}, —NR^{Q2CC}₂, —R^{Q2CM}, —NHC(═O)R^{Q2CC}, —NHC(═O)OR^{Q2CC}, or
   ═O.
(408) A compound according to any one of (1) to (406), wherein:
   each —R^{Q2C}, if present, is independently:
   —F,
   —R^{Q2CC}, —R^{Q2CX},
   —OH, —OR^{Q2CC}, —OR^{Q2CX},
   —NH₂, —NHR^{Q2CC}, —NR^{Q2CC}₂, or —R^{Q2CM}.
(409) A compound according to any one of (1) to (406), wherein:
   each —R92C, if present, is independently:
   —F,
   —R^{Q2CC}, —R^{Q2CX},
   OH, —OR^{Q2CC}, or —OR^{Q2CX},

### The Group —R^{Q2CC}

(410) A compound according to any one of (1) to (409), wherein:
   each —R^{Q2CC}, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(411) A compound according to any one of (1) to (409), wherein:
   each —RO2CC, if present, is independently linear or branched saturated C₁₋₄alkyl, phenyl, or phenyl-CH₂—.
(412) A compound according to any one of (1) to (409), wherein:
   each —RO2CC, if present, is independently linear or branched saturated C₁₋₄alkyl.
(413) A compound according to any one of (1) to (409), wherein:
   each —RO2CC, if present, is:
   -Me, -Et, -nPr, -iPr, -nBu, or -tBu.
(414) A compound according to any one of (1) to (409), wherein:
   each —R^{Q2CC}, if present, is:
   -Me, -Et, -nPr, or -iPr.
(415) A compound according to any one of (1) to (409), wherein:
   each —R92CC, if present, is:
   -Me or -Et.
(416) A compound according to any one of (1) to (409), wherein:
   each —R^{Q2CC}, if present, is:
   -Me.

### The Group —R^{Q2CX},

(417) A compound according to any one of (1) to (416), wherein:
   each —RO2CX, if present, is independently linear or branched saturated C1.4fluoroalkyl.
(418) A compound according to any one of (1) to (341), wherein:
   each —RO2CX, if present, is independently —CF₃, —CHF₂, —CH₂CF₃, or —CH₂CHF₂.
(419) A compound according to any one of (1) to (416), wherein:
   each —R^{Q2CX}, if present, is —CF₃.
      The Group —R^{Q2CM}, (420) A compound according to any one of (1) to (419), wherein:
each -RQ² CM, if present, is independently azetidino, pyrrolidino, piperidino, piperazino, morpholino, thiomorpholino, azepano, or diazepano, and is:
   optionally substituted on carbon with one or more groups —R^{Q2CMM}, and
   optionally substituted on secondary nitrogen, if present, with a group selected from: —R^{Q2CMM}, —C(═O)R^{Q2CMM}, —C(═O)OR^{Q2CMM}, —C(═O)NH₂, —C(═O)NHR^{Q2CMM}, —C(═O)NR92CMM2, and —S(═O)₂RQ2CMM
(421) A compound according to any one of (1) to (419), wherein:
   each —R^{Q2CM}, if present, is independently pyrrolidino, piperidino, piperazino, or morpholino, and is:
   optionally substituted on carbon with one or more groups —R^{Q2CMM}; and
   optionally substituted on secondary nitrogen, if present, with a group selected from: —R^{Q2CMM}, —C(═O)R^{Q2CMM}, —C(═O)OR^{Q2CMM}, —C(═O)NH₂, —C(═O)NHR^{Q2CMM}, —C(═O)NR^{Q2CMM}₂, and —S(═O)₂RQ2CMM
(422) A compound according to any one of (1) to (419), wherein:
   each -RQ² CM, if present, is independently pyrrolidino, piperidino, piperazino, or morpholino, and is:
   optionally substituted on carbon with one or more groups —R^{Q2CMM}; and
   optionally substituted on secondary nitrogen, if present, with a group selected from: —R^{Q2CMM}, —C(═O)R^{Q2CMM}, and —C(═O)ORQ2CMM

### The Group -RQ2CMM

(423) A compound according to any one of (1) to (422), wherein:
   each -RO2CMM, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(424) A compound according to any one of (1) to (422), wherein:
   each —R^{Q2CMM}, if present, is independently linear or branched saturated C₁₋₄alkyl, phenyl, or phenyl-CH₂—.
(425) A compound according to any one of (1) to (422), wherein:
   each —R^{Q2CMM}, if present, is independently linear or branched saturated C₁₋₄alkyl.
(426) A compound according to any one of (1) to (422), wherein:
   each —R^{Q2CMM}, if present, is:
   -Me, -Et, -nPr, -iPr, -nBu, or -tBu.
(427) A compound according to any one of (1) to (422), wherein:
   each —R^{Q2CMM}, if present, is:
   -Me, -Et, -nPr, or -iPr.
(428) A compound according to any one of (1) to (422), wherein:
   each -R02CMM, if present, is:
   -Me or -Et.
(429) A compound according to any one of (1) to (422), wherein:
   each —R^{Q2CMM}, if present, is:
   -Me.

### The Group —R^{Q2N}

(430) A compound according to any one of (1) to (429), wherein:
   each —R02N, if present, is independently:
   —R^{Q2NC}
   ═O, —C(═O)RQ2NC
   C(═O)-L^{Q2N}-R^{Q2NM},
   —C(═OOR^{Q2NC},
   -L^{Q2N}-C(═O)NR^{Q2NC}₂, or
   —S(═O)₂ROZNC.
(431) A compound according to any one of (1) to (429), wherein:
   each —R^{Q2N}; if present, is independently:
   —R^{Q2NC},
   —C(═O)R^{Q2NC},
   —C(═O-L^{Q2N}—OH, —C(═O)-LQ2N-ORQ2NC
   —C(═O)-L^{Q2N}-NH₂, —C(═O)-L^{Q2N}-NHRQ2NC, —C(═O)-L^{Q2N}-NRQ2NC2, —C(═O)-L^{Q2N}-R^{Q2NM},
   —C(═OOR^{Q2NC},
   —C(═O)NH₂, —C(═O)NHR^{Q2NC}, —C(═O)NR^{Q2NC}₂, —C(═O)RQ2NM
   -L^{Q2N}-C(═O)NH₂, -L^{Q2N}-C(═O)NHR^{Q2NC}, -L^{Q2N}-C(═O)NR^{Q2NC}₂, or -L^{Q2N}-C(═O)RQ2NM
(432) A compound according to any one of (1) to (429), wherein:
   each —R^{Q2N}, if present, is independently:
   —RQ2NC,
   —C(═O)RQ2NC
   —C(═O-L^{Q2N}—OH, —C(═O)-LQ2N-ORQ2NC
   —C(═O)-L^{Q2N}-NH₂, —C(═O)-L^{Q2N}-NHR^{Q2NC}, —C(═O)-L^{Q2N}-NR^{Q2NC}₂, —C(═O)-LQ2N-RO2NM
   —C(═O)NH₂, —C(═O)NHR^{Q2NC}, —C(═O)NR^{Q2NC2}, or —C(═O)R^{Q2NM}.
(433) A compound according to any one of (1) to (429), wherein:
   each —R^{Q2N}, if present, is independently:
   —R^{Q2NC} or
   —C(═O)R^{Q2NC}.
(434) A compound according to any one of (1) to (429), wherein:
   each —R92N, if present, is:
   —C(═O)R^{Q2NC}.

### The Group -RQ2NC

(435) A compound according to any one of (1) to (434), wherein:
   each —RO2NC, if present, is independently linear or branched saturated C1-6alkyl, phenyl-C₁₋₃alkyl, or C₅₋₆heteroaryl-C₁₋₃alkyl, wherein C1-6alkyl is optionally substituted with —OH, and each phenyl and heteroaryl is optionally substituted with one or more groups selected from: —CI, and —OCH₃.
(436) A compound according to any one of (1) to (434), wherein:
   each —RO2NC, if present, is independently -Me, -Et, -t-Bu, t-pentyl, benzyl, or pyridylmethyl, wherein-Me, -Et, -t-Bu, t-pentyl, is optionally substituted with —OH, and each phenyl and pyridyl is optionally substituted with one or more groups selected from: —CI, and —OCH₃.
(437) A compound according to any one of (1) to (434), wherein:
   each —RO2NC, if present, is independently linear or branched saturated C1-6alkyl, phenyl, phenyl-CH₂—, pyridyl, or pyridyl-CH₂—, wherein C1-6alkyl is optionally substituted with —OH or —OCH₃, and each phenyl and pyridyl is optionally substituted with one or more groups selected from: —F, —Cl, —CH₃, —CF₃, —OH, —OCH₃, —NH₂, —NH(CH₃), and —N(CH₃)₂;
(438) A compound according to any one of (1) to (434), wherein:
   each —RO2NC, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(439) A compound according to any one of (1) to (434), wherein:
   each —RO2NC, if present, is independently linear or branched saturated C₁₋₄alkyl, phenyl, or phenyl-CH₂—.
(440) A compound according to any one of (1) to (434), wherein:
   each —RO2NC, if present, is independently linear or branched saturated C₁₋₄alkyl.
(441) A compound according to any one of (1) to (434), wherein:
   each —RO2NC, if present, is:
   -Me, -Et, -nPr, -iPr, -nBu, or -tBu.
(442) A compound according to any one of (1) to (434), wherein:
   each —RO2NC, if present, is:
   -Me, -Et, -nPr, or -iPr.
(443) A compound according to any one of (1) to (434), wherein:
   each -RQ2NC, if present, is:
   -Me or -Et.
(444) A compound according to any one of (1) to (434), wherein:
   each -RQ2NC, if present, is:
   -Me.

### The Group -LQ2N—

(445) A compound according to any one of (1) to (444), wherein:
   each -LO2N—, if present, is independently —CH₂—, —C(CH₃)₂—, —CH₂CH₂—, or —CH₂CH₂CH₂—.
(446) A compound according to any one of (1) to (444), wherein:
   each -LO2N—, if present, is independently —CH₂—, —CH₂CH₂—, or —CH₂CH₂CH₂—.
(447) A compound according to any one of (1) to (444), wherein:
   each -LO2N—, if present, is —CH₂—.
(448) A compound according to any one of (1) to (444), wherein:
   each -LO2N—, if present, is —CH₂CH₂—.
(449) A compound according to any one of (1) to (444), wherein:
   each -L^{Q2N}-, if present, is —CH₂CH₂CH₂—.

### The Group —R^{Q2NM}

(450) A compound according to any one of (1) to (449), wherein:
   each -RO2NM, if present, is independently non-aromatic C₃₋₁₁heterocyclyl having at least one N ring atom, and is attached via that N ring atom.
(451) A compound according to any one of (1) to (449), wherein:
   each —R^{Q2NM}, if present, is independently pyrrolidinyl or morpholinyl attached via an N ring atom.
(452) A compound according to any one of (1) to (449), wherein:
   each -RO2NM, if present, is independently azetidino, pyrrolidino, piperidino, piperazino, morpholino, thiomorpholino, azepano, or diazepano, and is:
   optionally substituted on carbon with one or more groups —R^{Q2NMM}; and
   optionally substituted on secondary nitrogen, if present, with a group selected from: —R^{Q2NMM}, —C(═O)R^{Q2NM}, AM, —C(═O)OR^{Q2NMM}, —C(═O)NH₂, —C(═O)NHRQ2NMM —C(═O)NRQ2NMM2, and —S(═O)₂RQ2NMM
   (453) A compound according to any one of (1) to (449), wherein:
   each —R^{Q2NM}, if present, is independently pyrrolidino, piperidino, piperazino, or morpholino, and is:
   optionally substituted on carbon with one or more groups —R^{Q2NMM}; and optionally substituted on secondary nitrogen, if present, with a group selected from: —R^{Q2NMM}, —C(═O)R92NMM, —C(═O)OR^{Q2NMM}, —C(═O)NH₂, —C(═O)NHRQ2NMM —C(═O)NR^{Q2NMM}₂, and —S(═O)₂RQ2NMM
(454) A compound according to any one of (1) to (449), wherein:
   each -RO2NM, if present, is independently pyrrolidino, piperidino, piperazino, or morpholino, and is:
   optionally substituted on carbon with one or more groups —R^{Q2NMM}, and
   optionally substituted on secondary nitrogen, if present, with a group selected from: —R^{Q2NMM}, —C(═O)R^{Q2NMM}, and —C(═O)ORQ2NMM

### The Group -RQ2NMM

(455) A compound according to any one of (1) to (454), wherein:
   each —R^{Q2NMM}, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(456) A compound according to any one of (1) to (454), wherein:
   each —R^{Q2NMM}, if present, is independently linear or branched saturated C₁₋₄alkyl, phenyl, or phenyl-CH₂—.
(457) A compound according to any one of (1) to (454), wherein:
   each -RO2NMM, if present, is independently linear or branched saturated C₁₋₄alkyl.
(458) A compound according to any one of (1) to (454), wherein:
   each -R02NMM, if present, is:
   -Me, -Et, -nPr, -iPr, -nBu, or -tBu.
(459) A compound according to any one of (1) to (454), wherein:
   each —R^{Q2NMM}, if present, is:
   -Me, -Et, -nPr, or -iPr.
(460) A compound according to any one of (1) to (454), wherein:
   each —R^{Q2NMM}, if present, is:
   -Me or -Et.
(461) A compound according to any one of (1) to (454), wherein:
   each —R^{Q2NMM}, if present, is:
   -Me.

### The Group —R^{Q3CX} C

(462) A compound according to any one of (1) to (461), wherein:
   each —RO3C, if present, is independently:
   —F,
   —R^{Q3CX} CC
   —R^{Q3CX} CX,
   —OH, —OR^{Q3CC},
   —NHC(═O)R^{Q3CX} CC
   —C(═O)NHR^{Q3CC}, cc, —C(═O)RQ³ CM
   —S(═O)₂R^{Q3CX} CC or or —S(═O)₂RQ³ CM.
   and two adjacent-RG3C, if present, taken together may form —NH—(CH₂) 93C(O)(CH₂) v3—O—,
   wherein: q3 is 0 and v3 is 1.
(463) A compound according to any one of (1) to (461), wherein:
   each —RO3C, if present, is independently:
   —F, —Cl, —Br, —I,
   —R^{Q3CX} CC
   —R^{Q3CX} CX, —OR^{Q3CX} CX
   OH, —OR^{Q3CX} CC
   —NH₂, —NHR^{Q3CC}, —NRO3CC2, -RQ³ CM
   —NHC(═O)R^{Q3CC}, —NHC(═O)OR^{Q3CC},
   —C(═O)NH₂, —C(═O)NHR^{Q3CC}, —C(═O)NR^{Q3CXCC}₂, —C(═O)RQ³ CM
   —C(═O)OH, —C(═O)OR^{Q3CC},
   —OC(═O)RO3CC,
   —OC(═O)NH₂, —OC(═O)NHR^{Q3CC}, —OC(═O)NR^{Q3CG}₂, —OC(═O)RQ³ CM
   —CN, or —NO₂;
   and two adjacent-R^{Q3C}, if present, taken together may
   form —(CH₂)ₙ₃—O—(CH₂)ₘ₃—or —O—(CH₂)ₚ₃—O.
(464) A compound according to any one of (1) to (461), wherein:
   each —R^{Q3C}, if present, is independently:
   —F, —Cl, —Br, —I,
   —R^{Q3CC},
   —R^{Q3CX} CX, —OR^{Q3CX} CX
   —OH, —ORO3CC,
   —NH₂, —NHR^{Q3CC}, —NR^{Q3CCG}₂, or —R^{Q3CX} CM;
   and two adjacent-R^{Q3CX} C, if present, taken together may
form —(CH₂)ₙ₃—O—(CH₂)ₘ₃—or —O—(CH₂)ₚ₃—O.
(465) A compound according to any one of (1) to (461), wherein:
   each —R^{Q3CX} C, if present, is independently:
   —F, —Cl, —Br, —I,
   —R^{Q3CC},
   —R^{Q3CX} CX, —OR^{Q3CX} CX
   —OH, or —OR^{Q3CX} CC

The Indice “q3” in —(CH₂)_{q3} (C(O)—NH—(CH₂)ᵥ₃—and —NH—(CH₂)_{q3}C(O)(CH₂)ᵥ₃—O—.
(466) A compound according to any one of (1) to (465), wherein:
   q3, if present, is 0.
(467) A compound according to any one of (1) to (465), wherein:
   q3, if present, is 1.
(468) A compound according to any one of (1) to (465), wherein:
   q3, if present, is 2.
(469) A compound according to any one of (1) to (465), wherein:
   q3, if present, is 3.

The Indice “3” in —(CH₂)_{q3} (C(O)—NH—(CH₂)ᵥ₃—and —NH—(CH₂)_{q3}C(O)(CH₂)ᵥ₃—O—.
(470) A compound according to any one of (1) to (469), wherein:
   v3, if present, is 0.
(471) A compound according to any one of (1) to (469), wherein:
   v3, if present, is 1.
(472) A compound according to any one of (1) to (469), wherein:
   v3, if present, is 2.
(473) A compound according to any one of (1) to (469), wherein:
   v3, if present, is 3.

### The Group —R^{Q3CX} CC

(474) A compound according to any one of (1) to (473), wherein:
   each —RO3CC, if present, is independently linear or branched saturated C₁₋₄alkyl, C₃₋₆cycloalkyl, or C₃₋₇heterocyclyl, wherein C₁₋₄alkyl is optionally substituted with —F.
(475) A compound according to any one of (1) to (473), wherein:
   each —RO3CC, if present, is independently -Me, -Et, --iPr-iBu, cyclopropyl or 4,5-dihydro-1,3-oxazolyl, wherein-Me, -Et, --iPr, or -iBu is optionally substituted with —F.
(476) A compound according to any one of (1) to (473), wherein:
   each —RO3CC, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(477) A compound according to any one of (1) to (473), wherein:
   each —RO3CC, if present, is independently linear or branched saturated C₁₋₄alkyl, phenyl, or phenyl-CH₂—.
(478) A compound according to any one of (1) to (473), wherein:
   each —RO3CC, if present, is independently linear or branched saturated C₁₋₄alkyl.
(479) A compound according to any one of (1) to (473), wherein:
   each —R^{Q3CC}, if present, is:
   -Me, -Et, -nPr, -iPr, -nBu, or -tBu.
(480) A compound according to any one of (1) to (473), wherein:
   each —RO3CC, if present, is:
   -Me, -Et, -nPr, or -iPr.
(481) A compound according to any one of (1) to (473), wherein:
   each —RO3CC, if present, is:
   -Me or -Et.
(482) A compound according to any one of (1) to (473), wherein:
   each —RO3CC, if present, is:
   -Me.

### The Group —R^{Q3CX} CX

(483) A compound according to any one of (1) to (482), wherein:
   each —RO3CX, if present, is independently linear or branched saturated C₁₋₄fluoroalkyl.
(484) A compound according to any one of (1) to (482), wherein:
   each —RO3CX, if present, is independently —CF₃, —CHF₂, —CH₂CF₃, or —CH₂CHF₂.
(485) A compound according to any one of (1) to (482), wherein:
   each —RO3CX, if present, is —CF₃.

### The Group -L^{Q3C}-

(486) A compound according to any one of (1) to (485), wherein:
   each -L^{Q3C}-, if present, is independently —CH₂—, —C(CH₃)₂—, —CH₂CH₂—, or —CH₂CH₂CH₂—.
(487) A compound according to any one of (1) to (485), wherein:
   each -L^{Q3C}-, if present, is independently —CH₂—, —CH₂CH₂—, or —CH₂CH₂CH₂—.
(488) A compound according to any one of (1) to (485), wherein:
   each -L^{Q3C}-, if present, is —CH₂—.
(489) A compound according to any one of (1) to (485), wherein:
   each -L^{Q3C}-, if present, is —CH₂CH₂—.
(490) A compound according to any one of (1) to (485), wherein:
   each -L93G-, if present, is —CH₂CH₂CH₂—.

### The Group -RQ³ CM

(491) A compound according to any one of (1) to (490), wherein:
   each —RO3 CM, if present, is independently non-aromatic C₃₋₁₁heterocyclyl having at least one N ring atom, and is attached via that N ring atom; and is:
   optionally substituted on carbon with one or more groups —R^{Q3CCMM}.
(492) A compound according to any one of (1) to (490), wherein:
   each -RQ³ CM, if present, is independently pyrrolidinyl, or 2-azabicyclo[2.2.1]heptyl and is attached via an N ring atom; and is:
   optionally substituted on carbon with one or more groups —R^{Q3CCMM}.
(493) A compound according to any one of (1) to (490), wherein:
   each -RQ³ CM, if present, is independently azetidino, pyrrolidino, piperidino, piperazino, morpholino, thiomorpholino, azepano, or diazepano, and is:
   optionally substituted on carbon with one or more groups —R^{Q3CCMM}; and
   optionally substituted on secondary nitrogen, if present, with a group selected from: —R^{Q3CX} CM, —C(═O)RQ³ CM, —C(═O)ORQ³ CM, —C(═O)NH₂, —C(═O)NHRQ³ CM, —C(═O)NR^{Q3CXCMM}₂, and —S(═O)₂R^{Q3CCMM}.
(494) A compound according to any one of (1) to (490), wherein:
   each —RO3 CM, if present, is independently pyrrolidino, piperidino, piperazino, or morpholino, and is:
   optionally substituted on carbon with one or more groups —R^{Q3CCMM}; and
   optionally substituted on secondary nitrogen, if present, with a group selected from: —R^{Q3CCMM} —C(═O)R^{Q3CCMM}, —C(═O)OR^{Q3CX} CM, —C(═O)NH₂, —C(═O)NHRQ³ CM, —C(═ONR^{Q3CMM}₂, and —S(═O)₂R^{Q3CCMM}.
(495) A compound according to any one of (1) to (490), wherein:
   each —RO3 CM, if present, is independently pyrrolidino, piperidino, piperazino, or morpholino, and is:
   optionally substituted on carbon with one or more groups —R^{Q3CCMM}, and optionally substituted on secondary nitrogen, if present, with a group selected from: —R^{Q3CCMM}, —C(═O)R^{Q3CCMM}, and —C(═O)OR^{Q3CCMM}.

### The Group —R^{Q3CCMM}.

(496) A compound according to any one of (1) to (495), wherein:
   each -RO3CMM, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(497) A compound according to any one of (1) to (495), wherein:
   each -RO3CMM, if present, is independently linear or branched saturated C₁₋₄alkyl, phenyl, or phenyl-CH₂—.
(498) A compound according to any one of (1) to (495), wherein:
   each —R^{Q3CCMM}, if present, is independently linear or branched saturated C₁₋₄alkyl.
(499) A compound according to any one of (1) to (495), wherein:
   each -R93CMM, if present, is:
   -Me, -Et, -nPr, -iPr, -nBu, or -tBu.
(500) A compound according to any one of (1) to (495), wherein:
   each -R03CMM, if present, is:
   -Me, -Et, -nPr, or -iPr.
(501) A compound according to any one of (1) to (495), wherein:
   each -R03CMM, if present, is:
   -Me or -Et.
(502) A compound according to any one of (1) to (495), wherein:
   each —R^{Q3CCMM}, if present, is:
   -Me.
(503) A compound according to any one of (1) to (495), wherein:
   each —R^{Q3CCMM}, if present, is:
   —F.
(504) The Indices “n3” and “m3” in-(CH₂)ₙ₃—O—(CH₂)ₘ₃—and —(CH₂)ₙ₃—NH—(CH₂)ₘ₃-A compound according to any one of (1) to (503), wherein:
   n3, if present, is 0, 1, 2, or 3;
   m3, if present, is 0, 1, 2, or 3;
   with the proviso that m3+n3 is 2 or 3.
(505) A compound according to any one of (1) to (503), wherein:
   n3, if present, is 0, 1, or 2;
   m3, if present, is 0, 1, or 2;
   with the proviso that m3+n3 is 2 or 3.
(506) A compound according to any one of (1) to (503), wherein:
   n3, if present, is 1 or 2;
   m3, if present, is 1 or 2;
   with the proviso that m3+n3 is 2 or 3.

The Indices “p3” in-O—(CH₂)ₚ₃—O- and —NH—(CH₂)ₚ₃—NH—

(507) A compound according to any one of (1) to (503), wherein:
   p3, if present, is 1.
(508) A compound according to any one of (1) to (503), wherein:
   p3, if present, is 2.

### The Group -RQ4C

(509) A compound according to any one of (1) to (508), wherein:
   each -RQ4C, if present, is independently:
   —F,
   —RQ4CC—R^{Q4CX},
   —OH, —OR^{Q4CC}, -ORQ4CX
   —NH₂, —NHR Q⁴CC, —NR^{Q4CC}₂, or -RQ4CM
(510) A compound according to any one of (1) to (508), wherein:
   each —R94C, if present, is independently:
   —R°4CC, —R^{Q4CX},
   —OH, —OR94CC, or -ORQ4CX
(511) A compound according to any one of (1) to (508), wherein:
   each -RQ4C, if present, is independently:
   —RQ4CC
   —OH, or -ORQ4CC

### The Group -RQ4CC

(512) A compound according to any one of (1) to (511), wherein:
   each -ROACC, if present, is independently linear or branched saturated C₁₋₄alkyl, phenyl, phenyl-C₁₋₃alkyl, C₅₋₆heteroaryl, or C₅₋₆heteroaryl-C₁₋₃alkyl, wherein each phenyl and heteroaryl is optionally substituted with one or more groups selected from: —F, —Cl, —CH₃, —CF₃, —OH, —OCH₃, —NH₂, —NH(CH₃), and —N(CH₃)₂.
(513) A compound according to any one of (1) to (511), wherein:
   each —RO4CC, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(514) A compound according to any one of (1) to (511), wherein:
   each —R^{Q4CC}, if present, is independently linear or branched saturated C₁₋₄alkyl, phenyl, or phenyl-CH₂—.
(515) A compound according to any one of (1) to (511), wherein:
   each —RO4CC, if present, is independently linear or branched saturated C₁₋₄alkyl.
(516) A compound according to any one of (1) to (511), wherein:
   each —R^{Q4CC}, if present, is:
   -Me, -Et, -nPr, -iPr, -nBu, or -tBu.
(517) A compound according to any one of (1) to (511), wherein:
   each —RO4CC, if present, is:
   -Me, -Et, -nPr, or -iPr.
(518) A compound according to any one of (1) to (511), wherein:
   each —R^{Q4CC}, if present, is:
   Me or -Et.
(519) A compound according to any one of (1) to (511), wherein:
   each —R^{Q4CC}, if present, is:
   -Me.

### The Group -RQ4CX

(520) A compound according to any one of (1) to (519), wherein:
   each —RO4CX, if present, is independently linear or branched saturated C₁₋₄fluoroalkyl.
(521) A compound according to any one of (1) to (519), wherein:
   each —R94CX, if present, is independently —CF₃, —CHF₂, —CH₂CF₃, or —CH₂CHF₂.
(522) A compound according to any one of (1) to (519), wherein:
   each —R^{Q4CX}, if present, is —CF₃.

### The Group -RQ⁴ CM

(523) A compound according to any one of (1) to (522), wherein:
   each -RQ⁴ CM, if present, is independently azetidino, pyrrolidino, piperidino, piperazino, morpholino, thiomorpholino, azepano, or diazepano, and is:
   optionally substituted on carbon with one or more groups —R^{Q4CMM}; and optionally substituted on secondary nitrogen, if present, with a group selected from: —R^{Q4CMM}, —C(═O)R^{Q4CMM}, —C(═O)OR^{Q4CMM}, —C(═O)NH₂, —C(═O)NHRQ4CMM —C(═O)NRQ⁴ CM2, and —S(═O)₂RO4 CM
(524) A compound according to any one of (1) to (522), wherein:
   each -RQ⁴ CM, if present, is independently pyrrolidino, piperidino, piperazino, or morpholino, and is:
   optionally substituted on carbon with one or more groups —R^{Q4CMM}; and
   optionally substituted on secondary nitrogen, if present, with a group selected from: —R^{Q4CMM}, —C(═O)R^{Q4CMM}, —C(═O)OR^{Q4CMM}, —C(═O)NH₂, —C(═O)NHRQ4CMM —C(═O)NR^{Q4CMM}₂, and —S(═O)₂RQ4CMM
(525) A compound according to any one of (1) to (522), wherein:
   each -RQ⁴ CM, if present, is independently pyrrolidino, piperidino, piperazino, or morpholino, and is:
   optionally substituted on carbon with one or more groups -R94CMM; and
   optionally substituted on secondary nitrogen, if present, with a group selected from: —R^{Q4CMM}, —C(═O)R^{Q4CMM}, and —C(═O)ORQ4CMM

### The Group -RQ4CMM

(526) A compound according to any one of (1) to (522), wherein:
   each —R^{Q4CMM}, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(527) A compound according to any one of (1) to (522), wherein:
   each —R^{Q4CMM}, if present, is independently linear or branched saturated C₁₋₄alkyl, phenyl, or phenyl-CH₂—.
(528) A compound according to any one of (1) to (522), wherein:
each —R^{Q4CMM}, if present, is independently linear or branched saturated C₁₋₄alkyl.
(529) A compound according to any one of (1) to (522), wherein:
   each —R^{Q4CMM}, if present, is:
   -Me, -Et, -nPr, -iPr, -nBu, or -tBu.
(530) A compound according to any one of (1) to (522), wherein:
   each —R^{Q4CMM}, if present, is:
   -Me, -Et, -nPr, or -iPr.
(531) A compound according to any one of (1) to (522), wherein:
   each —R^{Q4CMM}, if present, is:
   -Me or -Et.
(532) A compound according to any one of (1) to (522), wherein:
   each —R^{Q4CMM}, if present, is:
   -Me.

### The Group -RQ5C

(533) A compound according to any one of (1) to (532), wherein:
   each -RQ5° C., if present, is independently:
   —OH, -ORQ5CC
   —NHC(═O)R^{Q5CC}, —NHC(═O)OR^{Q5CC},
   —C(═O)NH₂, —C(═O)NHR^{Q5CC}, or —C(═O)RQ⁵ CM.
(534) A compound according to any one of (1) to (532), wherein:
   each -RQ5C, if present, is independently:
   —OH, —OR^{Q5CC},
   —NH₂, —NHR Q⁵CC, —NR^{Q5CC}₂, -RQ⁵ CM,
   —NHC(═O)R05CC, —NHC(═O)ORQ5CC
   —C(═O)NH₂, —C(═O)NHR^{Q5CC}, —C(═O)NR^{Q5CC}₂, —C(═O)RQ⁵ CM
   —OC(═O)NH₂, —OC(═O)NHR^{Q5CC}, —OC(═O)NR^{Q5CC}₂, or —OC(═O)RQ⁵ CM
(535) A compound according to any one of (1) to (532), wherein:
   each —R95° C., if present, is independently:
   —OH, —OR^{Q5CC},
   —NH₂, —NHR Q⁵CC, —NR^{Q5CC}₂, -RQ⁵ CM
   —NHC(═O)R^{Q5CC}, or —NHC(═O)ORQ5CC
(536) A compound according to any one of (1) to (532), wherein:
   each —R95C, if present, is independently:
   —NH₂, —NHR^{Q5CC}, —NR^{Q5CC}₂, -RQ⁵ CM
   —NHC(═O)R^{Q5CC}, or —NHC(═O)ORQ5CC.
(537) A compound according to any one of (1) to (532), wherein:
   each -RQ5C, if present, is independently:
   —NHC(═O)R^{Q5CC} or —NHC(═O)ORQ5CC.

### The Group -RQ5CC

(538) A compound according to any one of (1) to (537), wherein:
   each —RO5CC, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(539) A compound according to any one of (1) to (537), wherein:
   each -RQ5CC, if present, is independently linear or branched saturated C₁₋₄alkyl, phenyl, or phenyl-CH₂—.
(540) A compound according to any one of (1) to (537), wherein:
   each —R05CC, if present, is independently linear or branched saturated C₁₋₄alkyl.
(541) A compound according to any one of (1) to (537), wherein:
   each —RO5CC, if present, is:
   -Me, -Et, -nPr, -iPr, -nBu, or -tBu.
(542) A compound according to any one of (1) to (537), wherein:
   each -RQ5CC, if present, is:
   -Me, -Et, or -tBu.
(543) A compound according to any one of (1) to (537), wherein:
   each -RQ5CC, if present, is:
   -Me, -Et, -nPr, or -iPr.
(544) A compound according to any one of (1) to (537), wherein:
   each -RQ5CC, if present, is:
   -Me or -Et.
(545) A compound according to any one of (1) to (537), wherein:
   each —R°5CC, if present, is:
   -Me.

### The Group -RQ⁵ CM

(546) A compound according to any one of (1) to (545), wherein:
   each -RQ⁵ CM, if present, is independently non-aromatic C₃₋₁₁heterocyclyl having at least one N ring atom, and is attached via that N ring atom.
(547) A compound according to any one of (1) to (545), wherein:
   each —RO5 CM, if present, is independently azetidino, pyrrolidino, piperidino, piperazino, morpholino, thiomorpholino, azepano, or diazepano, and is:
   optionally substituted on carbon with one or more groups —R^{Q5CMM}; and optionally substituted on secondary nitrogen, if present, with a group selected from: —R^{Q5CMM}, —C(═O)R^{Q5CMM}, —C(═O)OR^{Q5CMM}, —C(═O)NH₂, —C(═O)NHR^{Q5CMM}, —C(═O)NR^{Q5CMM}₂, and —S(═O)₂RQ5CMM
(548) A compound according to any one of (1) to (545), wherein:
   each -RQ⁵ CM, if present, is independently pyrrolidino, piperidino, piperazino, or morpholino, and is:
   optionally substituted on carbon with one or more groups —R^{Q5CMM}, and optionally substituted on secondary nitrogen, if present, with a group selected from: —R^{Q5CMM}, —C(═O)R^{Q5CMM}, —C(═O)OR^{Q5CMM}, —C(═O)NH₂, —C(═O)NHR^{Q5CMM}, —C(═O)NR^{Q5CMM}₂, and —S(═O)₂RQ5CMM
(549) A compound according to any one of (1) to (545), wherein:
   each -RQ⁵ CM, if present, is independently pyrrolidino, piperidino, piperazino, or morpholino, and is:
   optionally substituted on carbon with one or more groups —R^{Q5CMM}; and
   optionally substituted on secondary nitrogen, if present, with a group selected from: —R^{Q5CMM}, —C(═O)R05CMM, and —C(═O)ORQ5CMM
(550) A compound according to any one of (1) to (545), wherein:
   each -RQ⁵ CM, if present, is independently morpholino.

### The Group -RQ5CMM

(551) A compound according to any one of (1) to (550), wherein:
   each —R^{Q5CMM}, if present, is independently linear or branched saturated C₁₋₄alkyl, phenyl, or phenyl-CH₂—, wherein each phenyl is optionally substituted with one or more groups selected from: —F, —Cl, —CH₃, —CF₃, —OH, —OCH₃, —NH₂, —NH(CH₃), and —N(CH₃)₂.
(552) A compound according to any one of (1) to (550), wherein:
   each -RQ⁵ CM, if present, is independently linear or branched saturated C₁₋₄alkyl, phenyl, or phenyl-CH₂—.
(553) A compound according to any one of (1) to (550), wherein:
   each -RO5CMM, if present, is independently linear or branched saturated C₁₋₄alkyl.
(554) A compound according to any one of (1) to (550), wherein:
   each —R°5CMM, if present, is:
   -Me, -Et, -nPr, -iPr, -nBu, or -tBu.
(555) A compound according to any one of (1) to (550), wherein:
   each -RQ5CM, if present, is:
   -Me, -Et, -nPr, or -iPr.
(556) A compound according to any one of (1) to (550), wherein:
   each —R^{Q5CMM}, if present, is:
   -Me or -Et.
(557) A compound according to any one of (1) to (550), wherein:
   each —R^{Q5CMM}, if present, is:
   -Me.

### Certain Preferred Combinations

(558) A compound according to any one of (1) to (557), as applicable, wherein:
   —R^{A3} is —H; and
   —R^{A4} is —H.
For example, Ring A is:

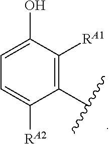

(559) A compound according to any one of (1) to (557), as applicable, wherein:
   —R^{A1} is —R^{A11}, R^{A11} is —R^{A111} and —R^{A111} is -Me;
   R^{A2} is —R^{A22}, —R^{A22} is —R^{A222}, and —R^{A222} is -Me;
   R^{A3} is —H; and
   —R^{A4} is —H.
For example, Ring A is:

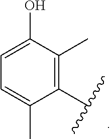 [SMILES: *c1c(C)ccc(O)c1C]

(560) A compound according to any one of (1) to (557), as applicable, wherein:

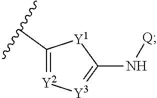

Ring B is
Y¹ is S;
Y² is CH or CR^{Y2};
Y³ is N;
—R^{A1} is —R^{A11}, R^{A11} is —R^{A111} and —R^{A111} is -Me;
—R^{A2} is —R^{A22}, —R^{A22} is —R^{A222}, and —R^{A222} is -Me;
R^{A3} is —H; and
—R^{A4} is —H.
For example, the compound is a compound having the following structural formula:

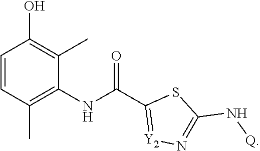

(561) A compound according to any one of (1) to (557), as applicable, wherein:
   Ring B is

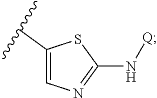

—R^{A1} is —R^{A11}, R^{A11} is —R^{A111} and —R^{A111} is -Me;
—R^{A2} is —R^{A22}, —R^{A22} is —R^{A222}, and —R^{A222} is -Me;
—R^{A3} is —H;
R^{A4} is —H; and
Q is -Q¹.
For example, the compound is a compound having the following structural formula:

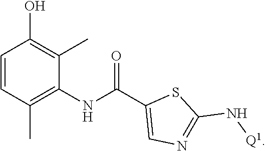

(562) A compound according to any one of (1) to (557), as applicable, wherein:
   Ring B is

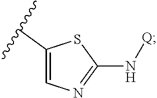

—R^{A1} is —R^{A11}, R^{A11} is —R^{A111} and —R^{A111} is -Me;
—R^{A2} is —R^{A22}, —R^{A22} is —R^{A222}, and —R^{A222} is -Me;
—R^{A3} is —H;
—R^{A4} is —H;
Q is -Q¹; and
Q¹ is pyrazolyl and is:
optionally substituted on carbon with one or more groups —R°1C; and
optionally substituted on secondary nitrogen, if present, with —R^{Q1N}.
(563) A compound according to any one of (1) to (557), as applicable, wherein:
   Ring B is

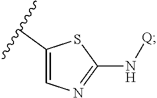

—R^{A1} is —R^{A11}, R^{A11} is —R^{A111} and —R^{A111} is -Me;
—R^{A2} is —R^{A2}, —R^{A22} is —R^{A22}, and —R^{A22} is -Me;
—R^{A3} is —H;
—R^{A4} is —H;
Q is -Q¹; and
-Q¹ is 1H-pyrazol-3-yl and is:
optionally substituted on carbon with one or more groups —R^{Q1C}; and
substituted on secondary nitrogen with —R^{Q1N}.
For example, the compound is a compound having the following structural formula:

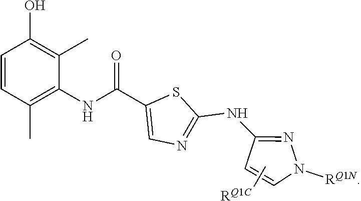

(564) A compound according to any one of (1) to (557), as applicable, wherein:
   Ring B is

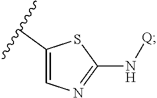

-Q is -Q¹; and
-Q¹ is pyridyl and is:
optionally substituted on carbon with one or more groups —R°1C; and
substituted on secondary nitrogen with —R^{Q1N}.
(565) A compound according to any one of (1) to (557), as applicable, wherein:
   Ring B is

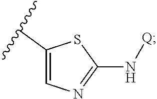

—R^{A1} is —R^{A11}, R^{A11} is —R^{A111} and —R^{A111} is -Me;
—R^{A2} is -RA22-R^{A2} is —R^{A22}, and —R^{A22} is -Me;
—R^{A3} is —H;
R^{A4} is —H;
Q is -Q¹; and
Q¹ is 1H-pyrazol-3-yl and is:
optionally substituted on carbon with one or more groups —R^{Q1C}; and
substituted on secondary nitrogen with —R^{Q1N};
wherein:
—R^{Q1N} is —R^{Q1NC}—R^{Q1Nhet}, -L^{Q1N}-C(═O)R^{Q1NP}, or -L^{Q1N}-C(═O)NR^{Q1NC}₂.
(566) A compound according to any one of (1) to (557), as applicable, wherein:
   Ring B is

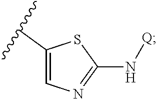

—R^{A1} is —R^{A11}, R^{A11} is —R^{A111} and —R^{A111} is -Me;
—R^{A2} is —R^{A22}, —R^{A22} is —R^{A222}, and —R^{A222} is -Me;
R^{A3} is —H;
R^{A4} is —H;
Q is -Q¹; and
Q¹ is 1H-pyrazol-3-yl and is:
optionally substituted on carbon with one or more groups —R^{Q1C}; and
substituted on secondary nitrogen with —R^{Q1N};
wherein:
—R^{Q1N} is -L^{Q1N}-C(═O)R^{Q1NP}; and
L^{Q1N}-is-CH₂—.
For example, the compound is a compound having the following structural formula:

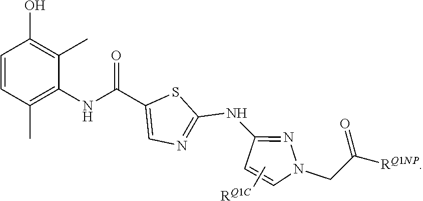

(567) A compound according to any one of (1) to (557), as applicable, wherein:
   Ring B is

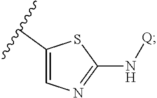

—R^{A1} is —R^{A11}, R^{A11} is —R^{A111} and —R^{A111} is -Me;
—R^{A2} is —R^{A22}, —R^{A22} is —R^{A222}, and —R^{A222} is -Me;
—R^{A3} is —H;
—R^{A4} is —H;
-Q is -Q¹; and
-Q¹ is 1H-pyrazol-3-yl and is:
optionally substituted on carbon with one or more groups —R^{Q1C}; and
substituted on secondary nitrogen with —R^{Q1N};
wherein:
R^{Q1N} is —R^{Q1NC}.
For example, the compound is a compound having the following structural formula:

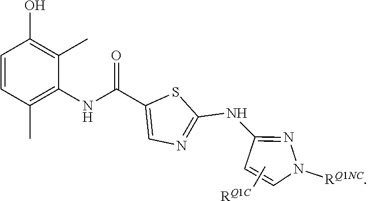

(568) A compound according to any one of (1) to (557), as applicable, wherein:
   Ring B is

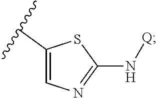

—R^{A1} is —R^{A11}, R^{A11} is —R^{A111} and —R^{A111} is -Me;
—R^{A2} is —R^{A22}, —R^{A22} is —R^{A222}, and —R^{A222} is -Me;
—R^{A3} is —H;
—R^{A4} is —H;
-Q is -Q¹; and
-Q¹ is 1H-pyrazol-3-yl and is:
substituted on secondary nitrogen with —R^{Q1N},
wherein:
—R^{Q1N} is —R^{Q1Nhet},
For example, the compound is a compound having the following structural formula:

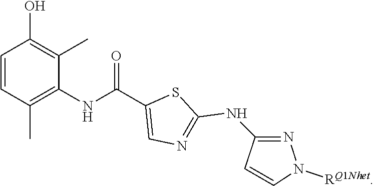

### Some Specific Examples

(569) A compound according to (1), selected from compounds of the following formulae and pharmaceutically acceptable salts and solvates thereof (e.g., and pharmaceutically acceptable salts thereof):

| # | Structure |
|---|---|
| BAA- 001 | 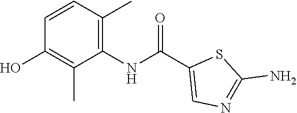 [SMILES: Cc1ccc(O)c(C)c1NC(=O)c1cnc(N)s1] |
| BAA- 002 | 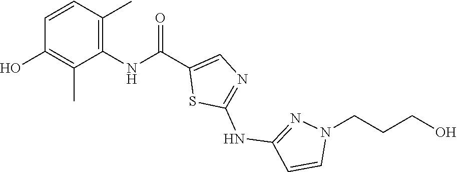 [SMILES: Cc1ccc(O)c(C)c1NC(=O)c1cnc(Nc2ccn(CCCO)n2)s1] |
| BAA- 003 | 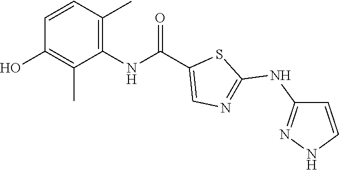 [SMILES: Cc1ccc(O)c(C)c1NC(=O)c1cnc(Nc2cc[nH]n2)s1] |
| BAA- 004 | 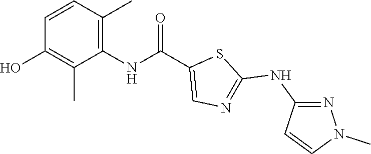 [SMILES: Cc1ccc(O)c(C)c1NC(=O)c1cnc(Nc2ccn(C)n2)s1] |
| BAA- 005 | 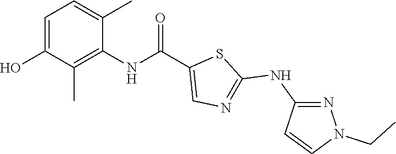 [SMILES: CCn1ccc(Nc2ncc(C(=O)Nc3c(C)ccc(O)c3C)s2)n1] |
| BAA- 006 | 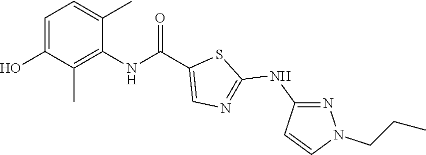 [SMILES: CCCn1ccc(Nc2ncc(C(=O)Nc3c(C)ccc(O)c3C)s2)n1] |
| BAA- 007 | 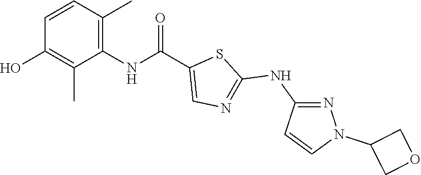 [SMILES: Cc1ccc(O)c(C)c1NC(=O)c1cnc(Nc2ccn(C3COC3)n2)s1] |
| BAA- 008 | 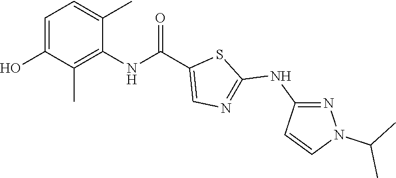 [SMILES: Cc1ccc(O)c(C)c1NC(=O)c1cnc(Nc2ccn(C(C)C)n2)s1] |
| BAA- 009 | 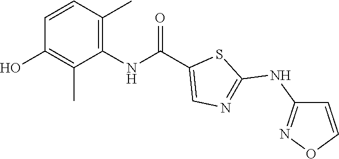 [SMILES: Cc1ccc(O)c(C)c1NC(=O)c1cnc(Nc2ccon2)s1] |
| BAA- 010 | 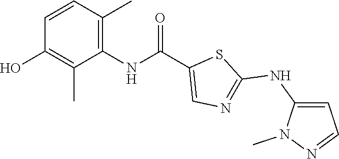 [SMILES: Cc1ccc(O)c(C)c1NC(=O)c1cnc(Nc2ccnn2C)s1] |
| BAA- 011 | 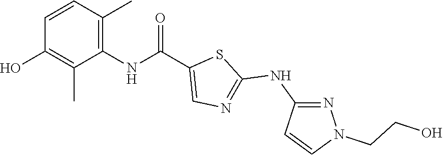 [SMILES: Cc1ccc(O)c(C)c1NC(=O)c1cnc(Nc2ccn(CCO)n2)s1] |
| BAA- 012 | 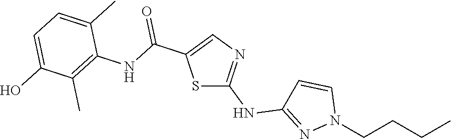 [SMILES: CCCCn1ccc(Nc2ncc(C(=O)Nc3c(C)ccc(O)c3C)s2)n1] |
| BAA- 013 | 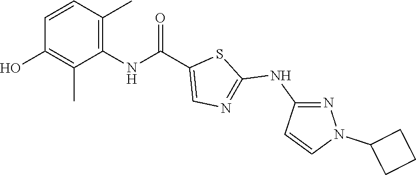 [SMILES: Cc1ccc(O)c(C)c1NC(=O)c1cnc(Nc2ccn(C3CCC3)n2)s1] |
| BAA- 014 | 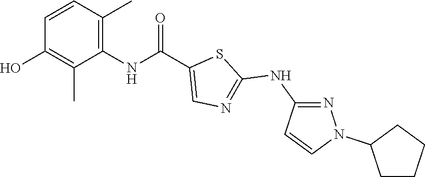 [SMILES: Cc1ccc(O)c(C)c1NC(=O)c1cnc(Nc2ccn(C3CCCC3)n2)s1] |
| BAA- 015 | 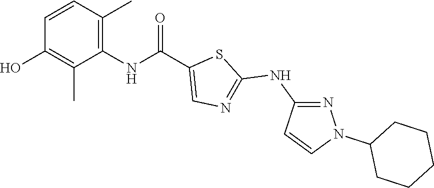 [SMILES: Cc1ccc(O)c(C)c1NC(=O)c1cnc(Nc2ccn(C3CCCCC3)n2)s1] |
| BAA- 016 | 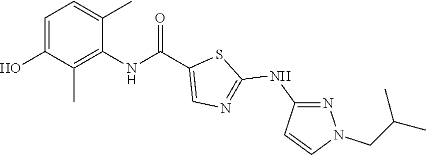 [SMILES: Cc1ccc(O)c(C)c1NC(=O)c1cnc(Nc2ccn(CC(C)C)n2)s1] |
| BAA- 017 | 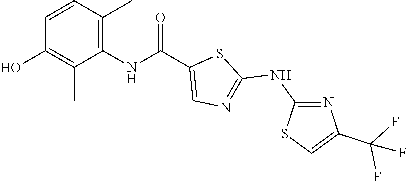 [SMILES: Cc1ccc(O)c(C)c1NC(=O)c1cnc(Nc2nc(C(F)(F)F)cs2)s1] |
| BAA- 018 | 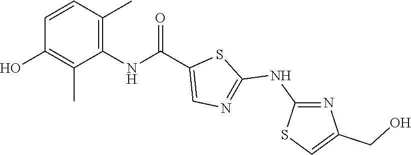 [SMILES: Cc1ccc(O)c(C)c1NC(=O)c1cnc(Nc2nc(CO)cs2)s1] |
| BAA- 019 | 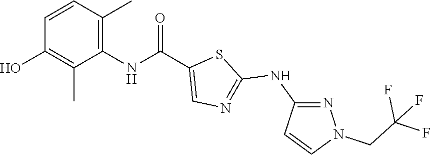 [SMILES: Cc1ccc(O)c(C)c1NC(=O)c1cnc(Nc2ccn(CC(F)(F)F)n2)s1] |
| BAA- 020 | 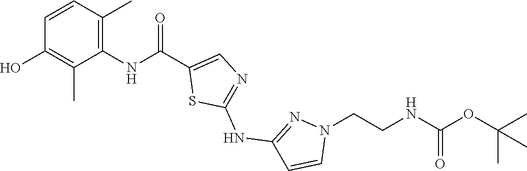 [SMILES: Cc1ccc(O)c(C)c1NC(=O)c1cnc(Nc2ccn(CCNC(=O)OC(C)(C)C)n2)s1] |
| BAA- 021 | 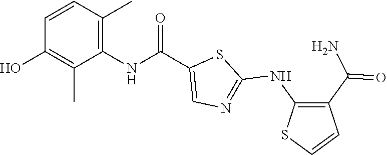 [SMILES: Cc1ccc(O)c(C)c1NC(=O)c1cnc(Nc2sccc2C(N)=O)s1] |
| BAA- 022 | 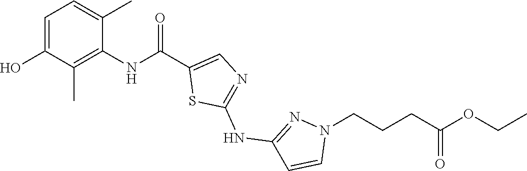 [SMILES: CCOC(=O)CCCn1ccc(Nc2ncc(C(=O)Nc3c(C)ccc(O)c3C)s2)n1] |
| BAA- 023 | 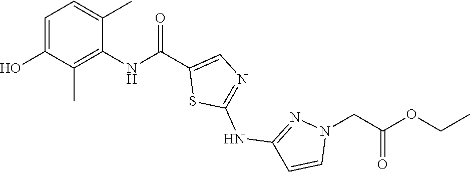 [SMILES: CCOC(=O)Cn1ccc(Nc2ncc(C(=O)Nc3c(C)ccc(O)c3C)s2)n1] |
| BAA- 024 | 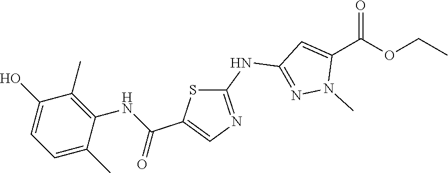 [SMILES: CCOC(=O)c1cc(Nc2ncc(C(=O)Nc3c(C)ccc(O)c3C)s2)nn1C] |
| BAA- 025 | 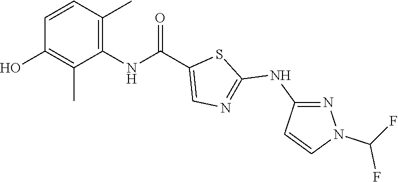 [SMILES: Cc1ccc(O)c(C)c1NC(=O)c1cnc(Nc2ccn(C(F)F)n2)s1] |
| BAA- 026 | 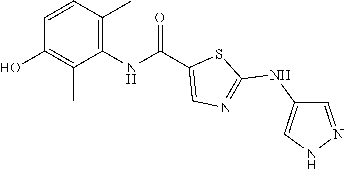 [SMILES: Cc1ccc(O)c(C)c1NC(=O)c1cnc(Nc2cn[nH]c2)s1] |
| BAA- 027 | 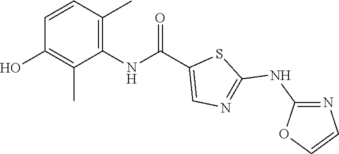 [SMILES: Cc1ccc(O)c(C)c1NC(=O)c1cnc(Nc2ncco2)s1] |
| BAA- 028 | 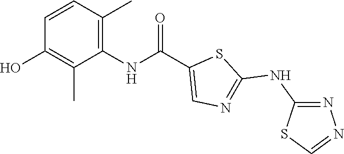 [SMILES: Cc1ccc(O)c(C)c1NC(=O)c1cnc(Nc2nncs2)s1] |
| BAA- 029 | 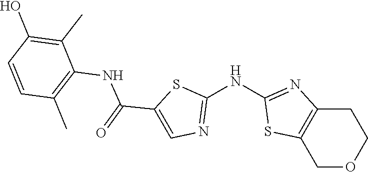 [SMILES: Cc1ccc(O)c(C)c1NC(=O)c1cnc(Nc2nc3c(s2)COCC3)s1] |
| BAA- 030 | 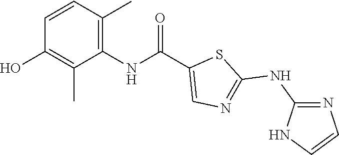 [SMILES: Cc1ccc(O)c(C)c1NC(=O)c1cnc(Nc2ncc[nH]2)s1] |
| BAA- 031 | 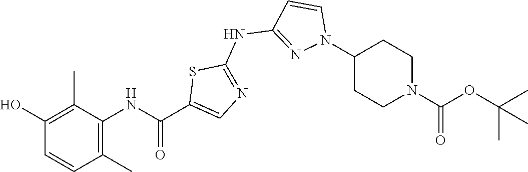 [SMILES: Cc1ccc(O)c(C)c1NC(=O)c1cnc(Nc2ccn(C3CCN(C(=O)OC(C)(C)C)CC3)n2)s1] |
| BAA- 032 | 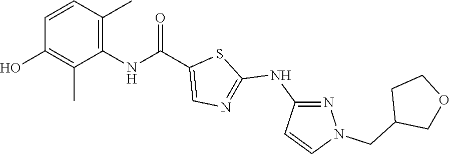 [SMILES: Cc1ccc(O)c(C)c1NC(=O)c1cnc(Nc2ccn(CC3CCOC3)n2)s1] |
| BAA- 033 | 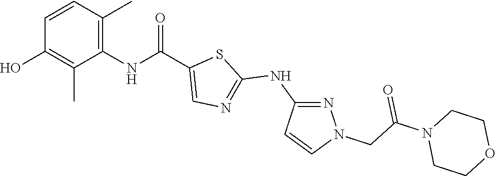 [SMILES: Cc1ccc(O)c(C)c1NC(=O)c1cnc(Nc2ccn(CC(=O)N3CCOCC3)n2)s1] |
| BAA- 034 | 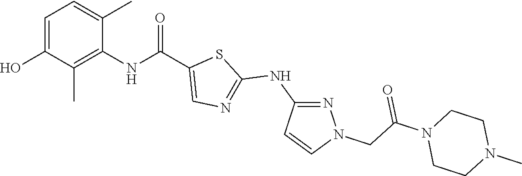 [SMILES: Cc1ccc(O)c(C)c1NC(=O)c1cnc(Nc2ccn(CC(=O)N3CCN(C)CC3)n2)s1] |
| BAA- 035 | 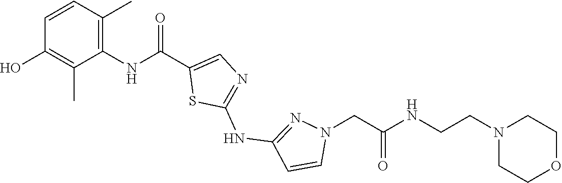 [SMILES: Cc1ccc(O)c(C)c1NC(=O)c1cnc(Nc2ccn(CC(=O)NCCN3CCOCC3)n2)s1] |
| BAA- 036 | 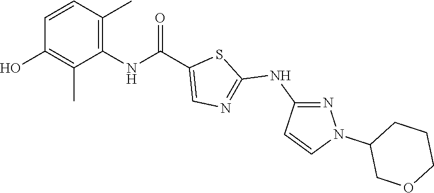 [SMILES: Cc1ccc(O)c(C)c1NC(=O)c1cnc(Nc2ccn(C3CCCOC3)n2)s1] |
| BAA- 037 | 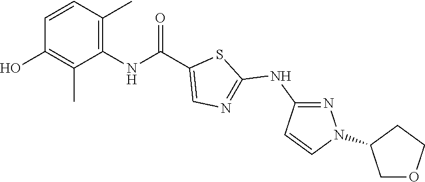 [SMILES: Cc1ccc(O)c(C)c1NC(=O)c1cnc(Nc2ccn([C@@H]3CCOC3)n2)s1] |
| BAA- 038 | 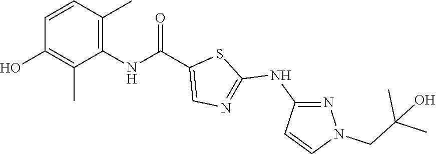 [SMILES: Cc1ccc(O)c(C)c1NC(=O)c1cnc(Nc2ccn(CC(C)(C)O)n2)s1] |
| BAA- 039 | 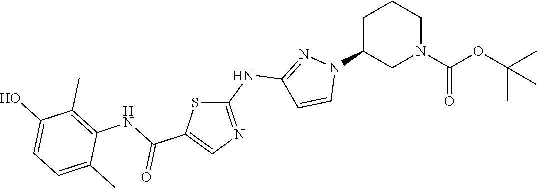 [SMILES: Cc1ccc(O)c(C)c1NC(=O)c1cnc(Nc2ccn([C@H]3CCCN(C(=O)OC(C)(C)C)C3)n2)s1] |
| BAA- 040 | 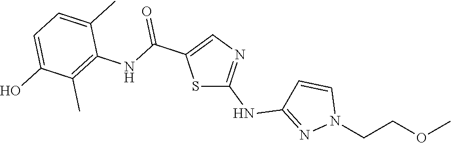 [SMILES: COCCn1ccc(Nc2ncc(C(=O)Nc3c(C)ccc(O)c3C)s2)n1] |
| BAA- 041 | 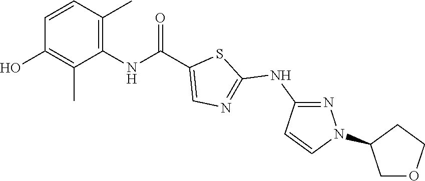 [SMILES: Cc1ccc(O)c(C)c1NC(=O)c1cnc(Nc2ccn([C@H]3CCOC3)n2)s1] |
| BAA- 042 | 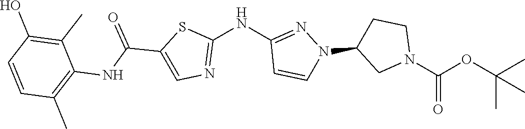 [SMILES: Cc1ccc(O)c(C)c1NC(=O)c1cnc(Nc2ccn([C@H]3CCN(C(=O)OC(C)(C)C)C3)n2)s1] |
| BAA- 043 | 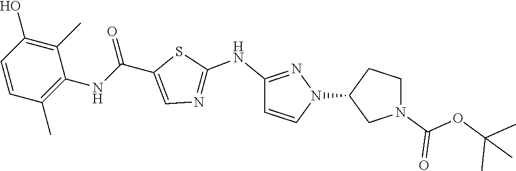 [SMILES: Cc1ccc(O)c(C)c1NC(=O)c1cnc(Nc2ccn([C@@H]3CCN(C(=O)OC(C)(C)C)C3)n2)s1] |
| BAA- 044 | 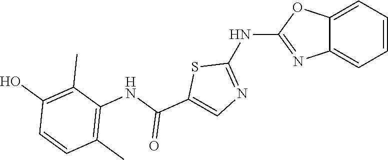 [SMILES: Cc1ccc(O)c(C)c1NC(=O)c1cnc(Nc2nc3ccccc3o2)s1] |
| BAA- 045 | 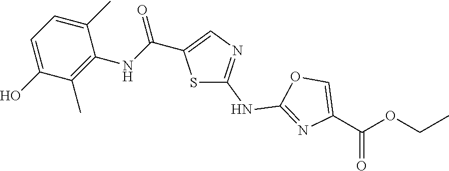 [SMILES: CCOC(=O)c1coc(Nc2ncc(C(=O)Nc3c(C)ccc(O)c3C)s2)n1] |
| BAA- 046 | 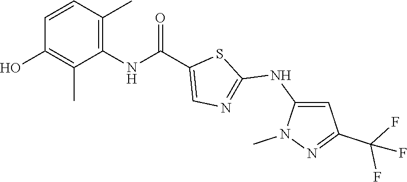 [SMILES: Cc1ccc(O)c(C)c1NC(=O)c1cnc(Nc2cc(C(F)(F)F)nn2C)s1] |
| BAA- 047 | 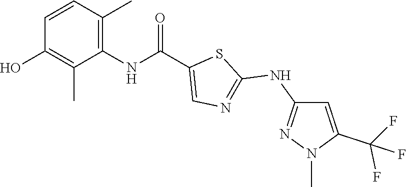 [SMILES: Cc1ccc(O)c(C)c1NC(=O)c1cnc(Nc2cc(C(F)(F)F)n(C)n2)s1] |
| BAA- 048 | 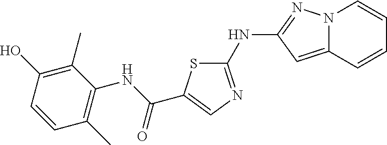 [SMILES: Cc1ccc(O)c(C)c1NC(=O)c1cnc(Nc2cc3ccccn3n2)s1] |
| BAA- 049 | 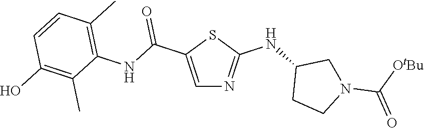 [SMILES: Cc1ccc(O)c(C)c1NC(=O)c1cnc(N[C@H]2CCN(C(=O)OC(C)(C)C)C2)s1] |
| BAA- 050 | 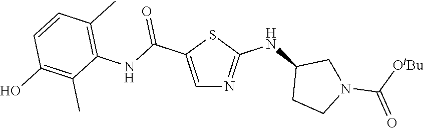 [SMILES: Cc1ccc(O)c(C)c1NC(=O)c1cnc(N[C@@H]2CCN(C(=O)OC(C)(C)C)C2)s1] |
| BAA- 051 | 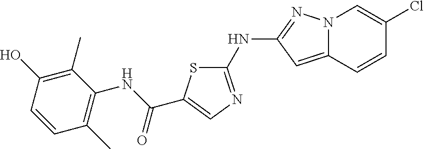 [SMILES: Cc1ccc(O)c(C)c1NC(=O)c1cnc(Nc2cc3ccc(Cl)cn3n2)s1] |
| BAA- 052 | 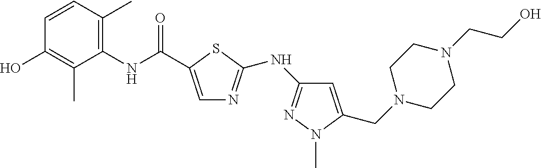 [SMILES: Cc1ccc(O)c(C)c1NC(=O)c1cnc(Nc2cc(CN3CCN(CCO)CC3)n(C)n2)s1] |
| BAA- 053 | 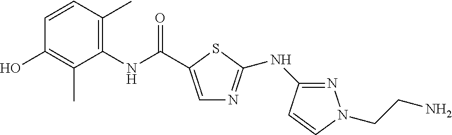 [SMILES: Cc1ccc(O)c(C)c1NC(=O)c1cnc(Nc2ccn(CCN)n2)s1] |
| BAA- 054 | 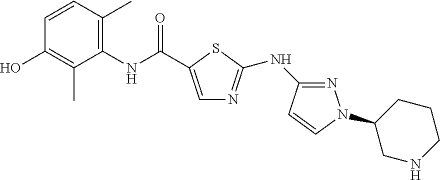 [SMILES: Cc1ccc(O)c(C)c1NC(=O)c1cnc(Nc2ccn([C@H]3CCCNC3)n2)s1] |
| BAA- 055 | 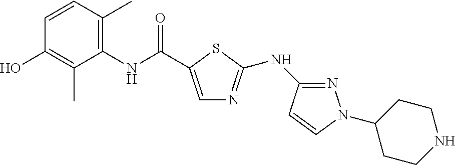 [SMILES: Cc1ccc(O)c(C)c1NC(=O)c1cnc(Nc2ccn(C3CCNCC3)n2)s1] |
| BAA- 056 | 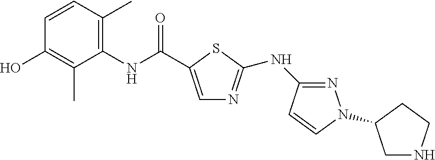 [SMILES: Cc1ccc(O)c(C)c1NC(=O)c1cnc(Nc2ccn([C@@H]3CCNC3)n2)s1] |
| BAA- 057 | 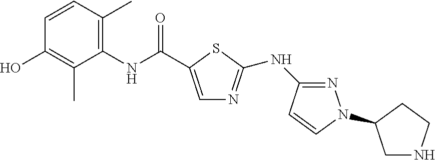 [SMILES: Cc1ccc(O)c(C)c1NC(=O)c1cnc(Nc2ccn([C@H]3CCNC3)n2)s1] |
| BAA- 058 | 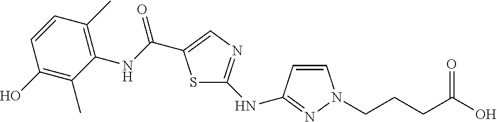 [SMILES: Cc1ccc(O)c(C)c1NC(=O)c1cnc(Nc2ccn(CCCC(=O)O)n2)s1] |
| BAA- 059 | 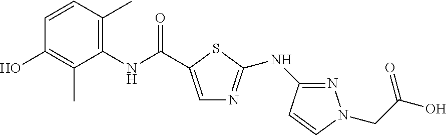 [SMILES: Cc1ccc(O)c(C)c1NC(=O)c1cnc(Nc2ccn(CC(=O)O)n2)s1] |
| BAA- 060 | 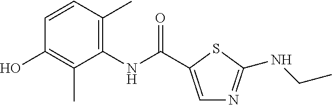 [SMILES: CCNc1ncc(C(=O)Nc2c(C)ccc(O)c2C)s1] |
| BAA- 061 | 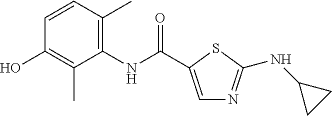 [SMILES: Cc1ccc(O)c(C)c1NC(=O)c1cnc(NC2CC2)s1] |
| BAA- 062 | 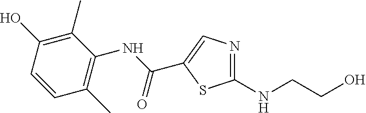 [SMILES: Cc1ccc(O)c(C)c1NC(=O)c1cnc(NCCO)s1] |
| BAA- 063 | 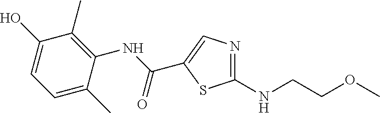 [SMILES: COCCNc1ncc(C(=O)Nc2c(C)ccc(O)c2C)s1] |
| BAA- 064 | 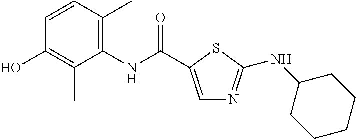 [SMILES: Cc1ccc(O)c(C)c1NC(=O)c1cnc(NC2CCCCC2)s1] |
| BAA- 065 | 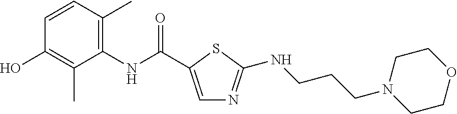 [SMILES: Cc1ccc(O)c(C)c1NC(=O)c1cnc(NCCCN2CCOCC2)s1] |
| BAA- 066 | 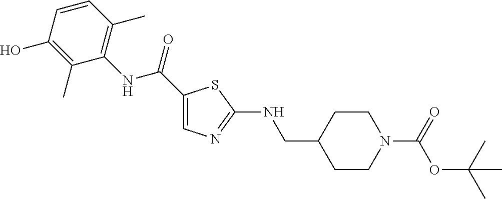 [SMILES: Cc1ccc(O)c(C)c1NC(=O)c1cnc(NCC2CCN(C(=O)OC(C)(C)C)CC2)s1] |
| BAA- 067 | 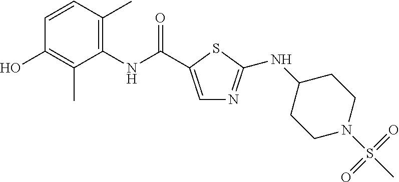 [SMILES: Cc1ccc(O)c(C)c1NC(=O)c1cnc(NC2CCN(S(C)(=O)=O)CC2)s1] |
| BAA- 068 | 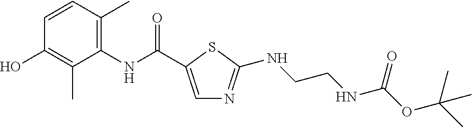 [SMILES: Cc1ccc(O)c(C)c1NC(=O)c1cnc(NCCNC(=O)OC(C)(C)C)s1] |
| BAA- 069 | 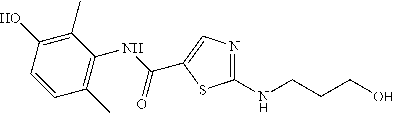 [SMILES: Cc1ccc(O)c(C)c1NC(=O)c1cnc(NCCCO)s1] |
| BAA- 070 | 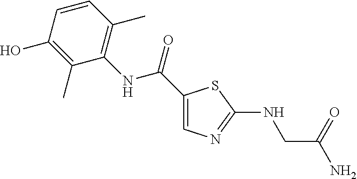 [SMILES: Cc1ccc(O)c(C)c1NC(=O)c1cnc(NCC(N)=O)s1] |
| BAA- 071 | 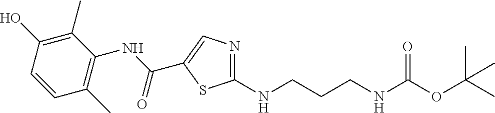 [SMILES: Cc1ccc(O)c(C)c1NC(=O)c1cnc(NCCCNC(=O)OC(C)(C)C)s1] |
| BAA- 072 | 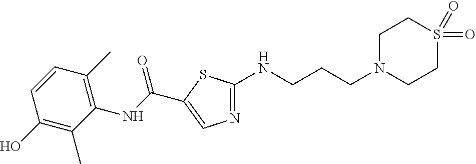 [SMILES: Cc1ccc(O)c(C)c1NC(=O)c1cnc(NCCCN2CCS(=O)(=O)CC2)s1] |
| BAA- 073 | 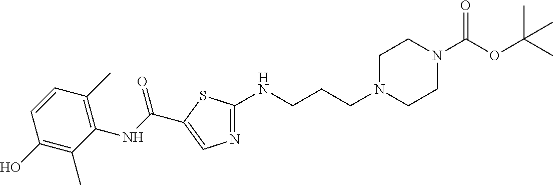 [SMILES: Cc1ccc(O)c(C)c1NC(=O)c1cnc(NCCCN2CCN(C(=O)OC(C)(C)C)CC2)s1] |
| BAA- 074 | 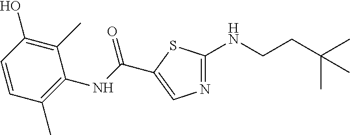 [SMILES: Cc1ccc(O)c(C)c1NC(=O)c1cnc(NCCC(C)(C)C)s1] |
| BAA- 075 | 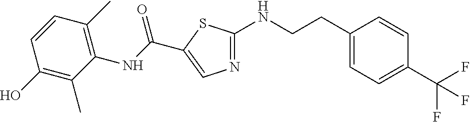 [SMILES: Cc1ccc(O)c(C)c1NC(=O)c1cnc(NCCc2ccc(C(F)(F)F)cc2)s1] |
| BAA- 076 | 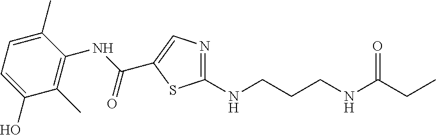 [SMILES: CCC(=O)NCCCNc1ncc(C(=O)Nc2c(C)ccc(O)c2C)s1] |
| BAA- 077 | 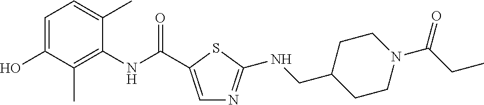 [SMILES: CCC(=O)N1CCC(CNc2ncc(C(=O)Nc3c(C)ccc(O)c3C)s2)CC1] |
| BAA- 078 | 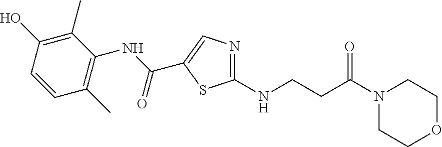 [SMILES: Cc1ccc(O)c(C)c1NC(=O)c1cnc(NCCC(=O)N2CCOCC2)s1] |
| BAA- 079 | 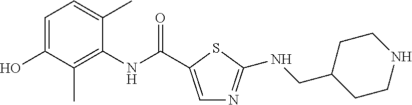 [SMILES: Cc1ccc(O)c(C)c1NC(=O)c1cnc(NCC2CCNCC2)s1] |
| BAA- 080 | 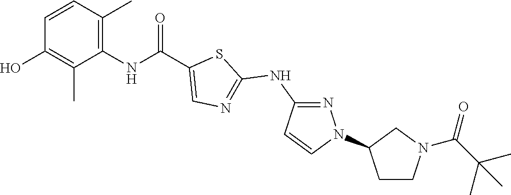 [SMILES: Cc1ccc(O)c(C)c1NC(=O)c1cnc(Nc2ccn([C@@H]3CCN(C(=O)C(C)(C)C)C3)n2)s1] |
| BAA- 081 | 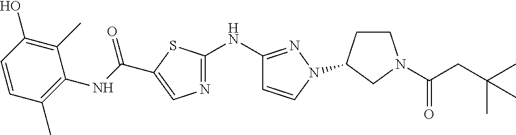 [SMILES: Cc1ccc(O)c(C)c1NC(=O)c1cnc(Nc2ccn([C@@H]3CCN(C(=O)CC(C)(C)C)C3)n2)s1] |
| BAA- 082 | 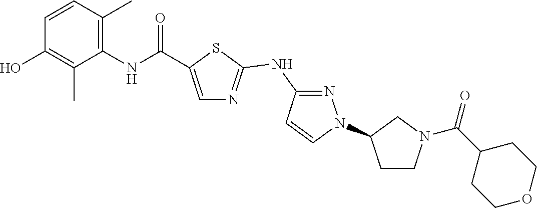 [SMILES: Cc1ccc(O)c(C)c1NC(=O)c1cnc(Nc2ccn([C@@H]3CCN(C(=O)C4CCOCC4)C3)n2)s1] |
| BAA- 083 | 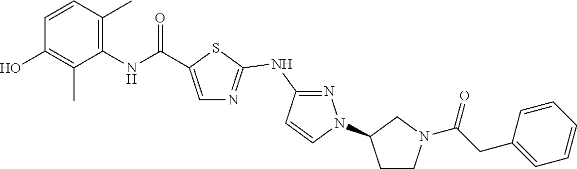 [SMILES: Cc1ccc(O)c(C)c1NC(=O)c1cnc(Nc2ccn([C@@H]3CCN(C(=O)Cc4ccccc4)C3)n2)s1] |
| BAA- 084 | 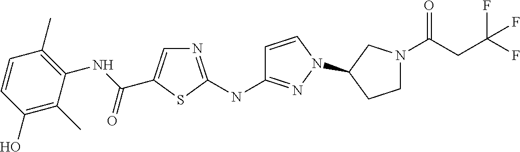 [SMILES: Cc1ccc(O)c(C)c1NC(=O)c1cnc(Nc2ccn([C@@H]3CCN(C(=O)CC(F)(F)F)C3)n2)s1] |
| BAA- 085 | 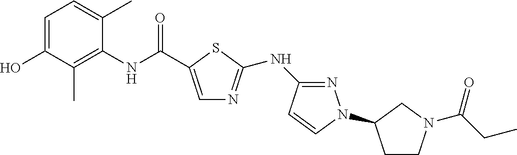 [SMILES: CCC(=O)N1CC[C@@H](n2ccc(Nc3ncc(C(=O)Nc4c(C)ccc(O)c4C)s3)n2)C1] |
| BAA- 086 | 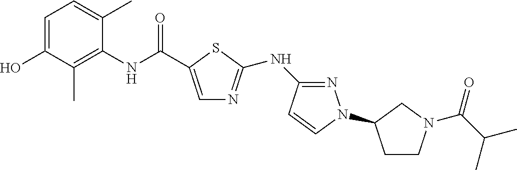 [SMILES: Cc1ccc(O)c(C)c1NC(=O)c1cnc(Nc2ccn([C@@H]3CCN(C(=O)C(C)C)C3)n2)s1] |
| BAA- 087 | 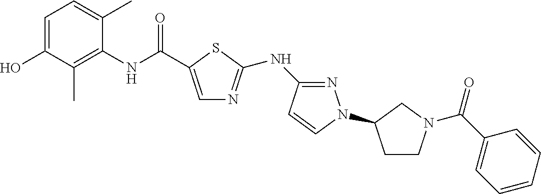 [SMILES: Cc1ccc(O)c(C)c1NC(=O)c1cnc(Nc2ccn([C@@H]3CCN(C(=O)c4ccccc4)C3)n2)s1] |
| BAA- 087 | 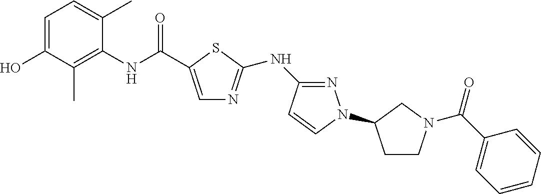 [SMILES: Cc1ccc(O)c(C)c1NC(=O)c1cnc(Nc2ccn([C@@H]3CCN(C(=O)c4ccccc4)C3)n2)s1] |
| BAA- 088 | 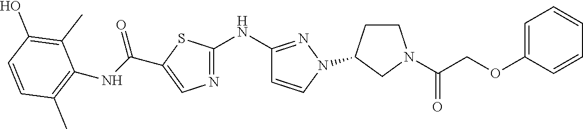 [SMILES: Cc1ccc(O)c(C)c1NC(=O)c1cnc(Nc2ccn([C@@H]3CCN(C(=O)COc4ccccc4)C3)n2)s1] |
| BAA- 089 | 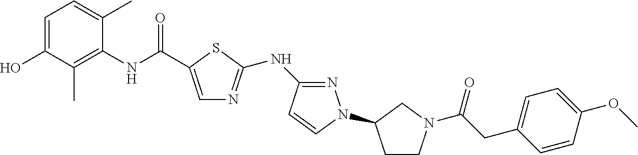 [SMILES: COc1ccc(CC(=O)N2CC[C@@H](n3ccc(Nc4ncc(C(=O)Nc5c(C)ccc(O)c5C)s4)n3)C2)cc1] |
| BAA- 090 | 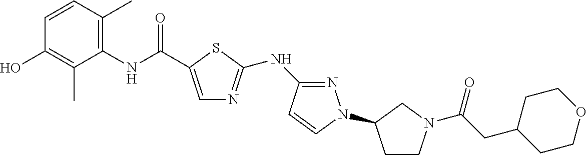 [SMILES: Cc1ccc(O)c(C)c1NC(=O)c1cnc(Nc2ccn([C@@H]3CCN(C(=O)CC4CCOCC4)C3)n2)s1] |
| BAA- 091 | 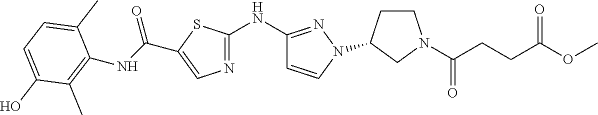 [SMILES: COC(=O)CCC(=O)N1CC[C@@H](n2ccc(Nc3ncc(C(=O)Nc4c(C)ccc(O)c4C)s3)n2)C1] |
| BAA- 092 | 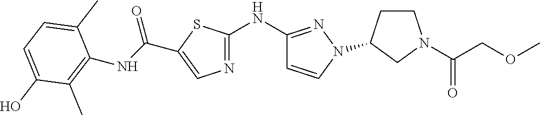 [SMILES: COCC(=O)N1CC[C@@H](n2ccc(Nc3ncc(C(=O)Nc4c(C)ccc(O)c4C)s3)n2)C1] |
| BAA- 093 | 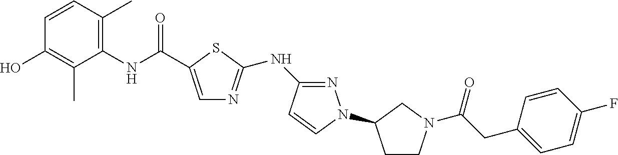 [SMILES: Cc1ccc(O)c(C)c1NC(=O)c1cnc(Nc2ccn([C@@H]3CCN(C(=O)Cc4ccc(F)cc4)C3)n2)s1] |
| BAA- 094 | 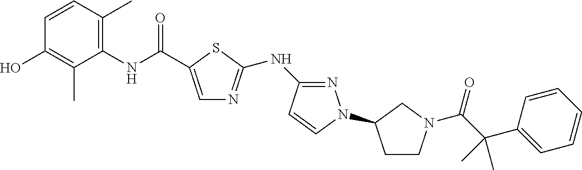 [SMILES: Cc1ccc(O)c(C)c1NC(=O)c1cnc(Nc2ccn([C@@H]3CCN(C(=O)C(C)(C)c4ccccc4)C3)n2)s1] |
| BAA- 095 | 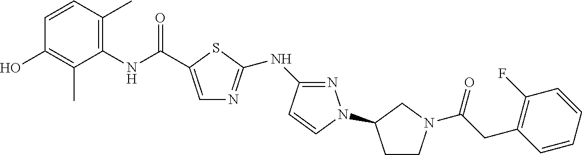 [SMILES: Cc1ccc(O)c(C)c1NC(=O)c1cnc(Nc2ccn([C@@H]3CCN(C(=O)Cc4ccccc4F)C3)n2)s1] |
| BAA- 096 | 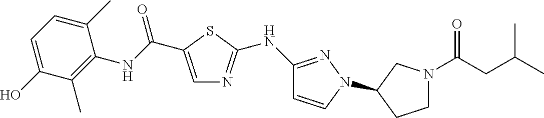 [SMILES: Cc1ccc(O)c(C)c1NC(=O)c1cnc(Nc2ccn([C@@H]3CCN(C(=O)CC(C)C)C3)n2)s1] |
| BAA- 097 | 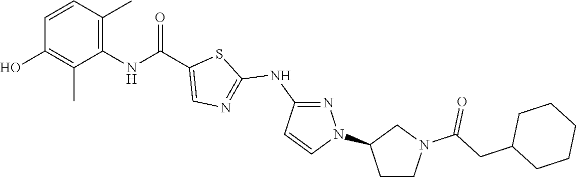 [SMILES: Cc1ccc(O)c(C)c1NC(=O)c1cnc(Nc2ccn([C@@H]3CCN(C(=O)CC4CCCCC4)C3)n2)s1] |
| BAA- 098 | 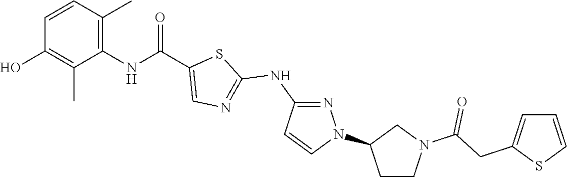 [SMILES: Cc1ccc(O)c(C)c1NC(=O)c1cnc(Nc2ccn([C@@H]3CCN(C(=O)Cc4cccs4)C3)n2)s1] |
| BAA- 099 | 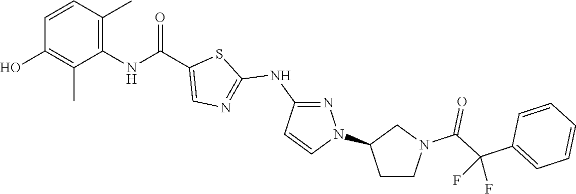 [SMILES: Cc1ccc(O)c(C)c1NC(=O)c1cnc(Nc2ccn([C@@H]3CCN(C(=O)C(F)(F)c4ccccc4)C3)n2)s1] |
| BAA- 100 | 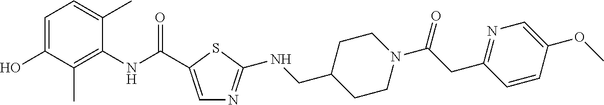 [SMILES: COc1ccc(CC(=O)N2CCC(CNc3ncc(C(=O)Nc4c(C)ccc(O)c4C)s3)CC2)nc1] |
| BAA- 101 | 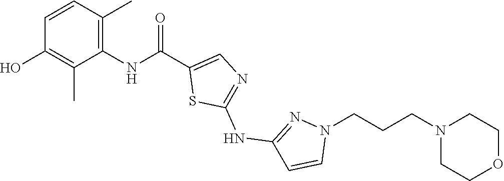 [SMILES: Cc1ccc(O)c(C)c1NC(=O)c1cnc(Nc2ccn(CCCN3CCOCC3)n2)s1] |
| BAA- 102 | 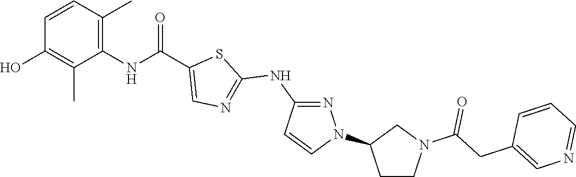 [SMILES: Cc1ccc(O)c(C)c1NC(=O)c1cnc(Nc2ccn([C@@H]3CCN(C(=O)Cc4cccnc4)C3)n2)s1] |
| BAA- 103 | 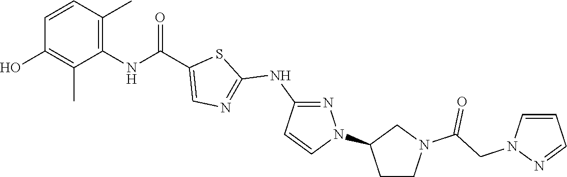 [SMILES: Cc1ccc(O)c(C)c1NC(=O)c1cnc(Nc2ccn([C@@H]3CCN(C(=O)Cn4cccn4)C3)n2)s1] |
| BAA- 104 | 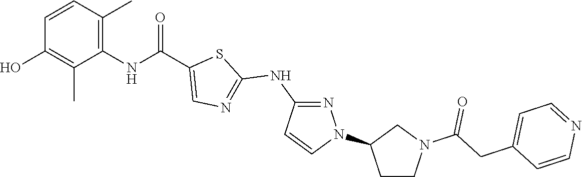 [SMILES: Cc1ccc(O)c(C)c1NC(=O)c1cnc(Nc2ccn([C@@H]3CCN(C(=O)Cc4ccncc4)C3)n2)s1] |
| BAA- 105 | 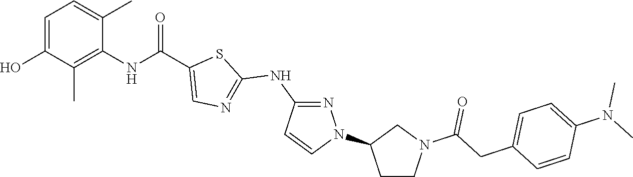 [SMILES: Cc1ccc(O)c(C)c1NC(=O)c1cnc(Nc2ccn([C@@H]3CCN(C(=O)Cc4ccc(N(C)C)cc4)C3)n2)s1] |
| BAA- 106 | 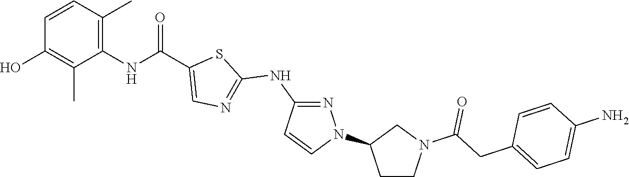 [SMILES: Cc1ccc(O)c(C)c1NC(=O)c1cnc(Nc2ccn([C@@H]3CCN(C(=O)Cc4ccc(N)cc4)C3)n2)s1] |
| BAA- 107 | 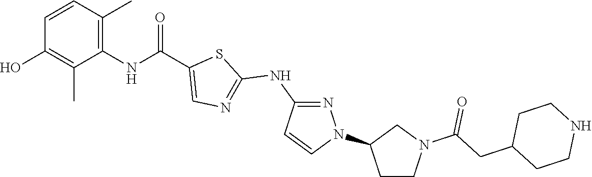 [SMILES: Cc1ccc(O)c(C)c1NC(=O)c1cnc(Nc2ccn([C@@H]3CCN(C(=O)CC4CCNCC4)C3)n2)s1] |
| BAA- 108 | 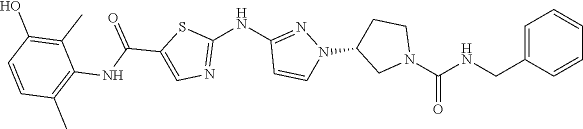 [SMILES: Cc1ccc(O)c(C)c1NC(=O)c1cnc(Nc2ccn([C@@H]3CCN(C(=O)NCc4ccccc4)C3)n2)s1] |
| BAA- 109 | 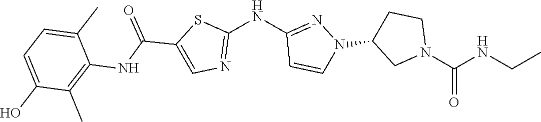 [SMILES: CCNC(=O)N1CC[C@@H](n2ccc(Nc3ncc(C(=O)Nc4c(C)ccc(O)c4C)s3)n2)C1] |
| BAA- 110 | 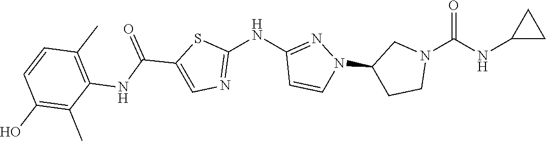 [SMILES: Cc1ccc(O)c(C)c1NC(=O)c1cnc(Nc2ccn([C@@H]3CCN(C(=O)NC4CC4)C3)n2)s1] |
| BAA- 111 | 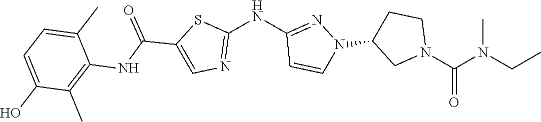 [SMILES: CCN(C)C(=O)N1CC[C@@H](n2ccc(Nc3ncc(C(=O)Nc4c(C)ccc(O)c4C)s3)n2)C1] |
| BAA- 112 | 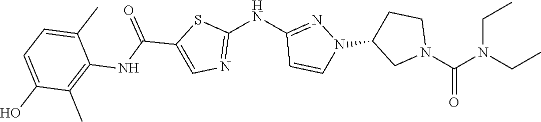 [SMILES: CCN(CC)C(=O)N1CC[C@@H](n2ccc(Nc3ncc(C(=O)Nc4c(C)ccc(O)c4C)s3)n2)C1] |
| BAA- 113 | 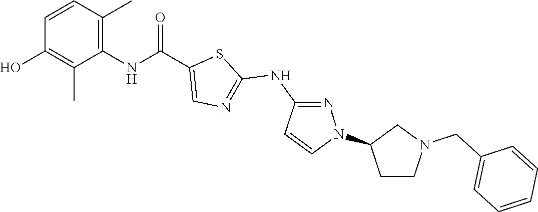 [SMILES: Cc1ccc(O)c(C)c1NC(=O)c1cnc(Nc2ccn([C@@H]3CCN(Cc4ccccc4)C3)n2)s1] |
| BAA- 114 | 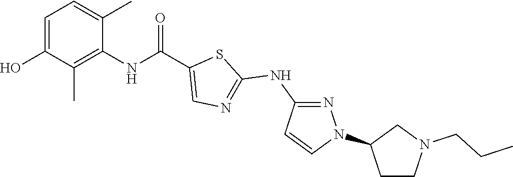 [SMILES: CCCN1CC[C@@H](n2ccc(Nc3ncc(C(=O)Nc4c(C)ccc(O)c4C)s3)n2)C1] |
| BAA- 115 | 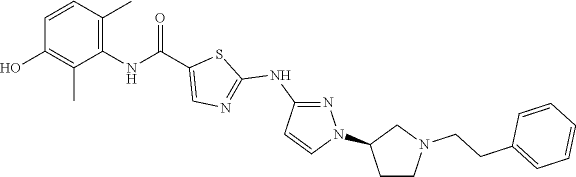 [SMILES: Cc1ccc(O)c(C)c1NC(=O)c1cnc(Nc2ccn([C@@H]3CCN(CCc4ccccc4)C3)n2)s1] |
| BAA- 116 | 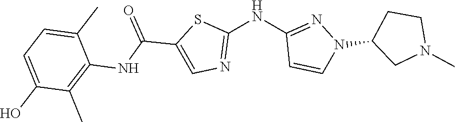 [SMILES: Cc1ccc(O)c(C)c1NC(=O)c1cnc(Nc2ccn([C@@H]3CCN(C)C3)n2)s1] |
| BAA- 117 | 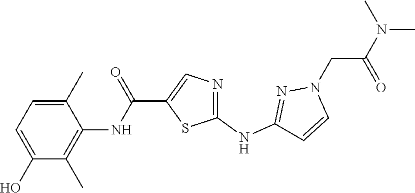 [SMILES: Cc1ccc(O)c(C)c1NC(=O)c1cnc(Nc2ccn(CC(=O)N(C)C)n2)s1] |
| BAA- 118 | 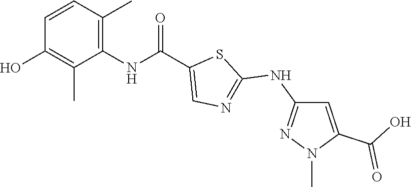 [SMILES: Cc1ccc(O)c(C)c1NC(=O)c1cnc(Nc2cc(C(=O)O)n(C)n2)s1] |
| BAA- 119 | 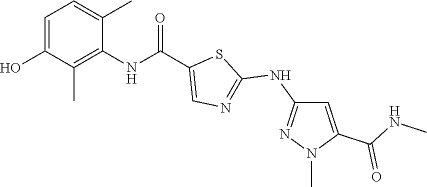 [SMILES: CNC(=O)c1cc(Nc2ncc(C(=O)Nc3c(C)ccc(O)c3C)s2)nn1C] |
| BAA- 120 | 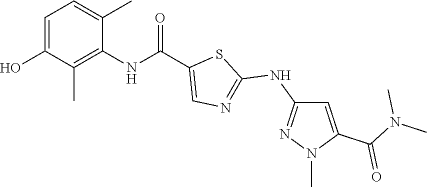 [SMILES: Cc1ccc(O)c(C)c1NC(=O)c1cnc(Nc2cc(C(=O)N(C)C)n(C)n2)s1] |
| BAA- 121 | 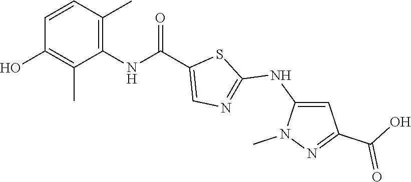 [SMILES: Cc1ccc(O)c(C)c1NC(=O)c1cnc(Nc2cc(C(=O)O)nn2C)s1] |
| BAA- 122 | 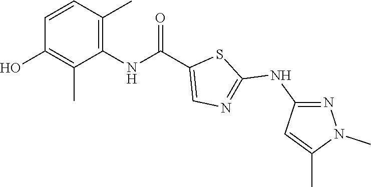 [SMILES: Cc1ccc(O)c(C)c1NC(=O)c1cnc(Nc2cc(C)n(C)n2)s1] |
| BAA- 123 | 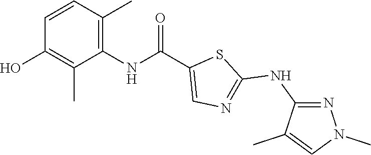 [SMILES: Cc1cn(C)nc1Nc1ncc(C(=O)Nc2c(C)ccc(O)c2C)s1] |
| BAA- 124 | 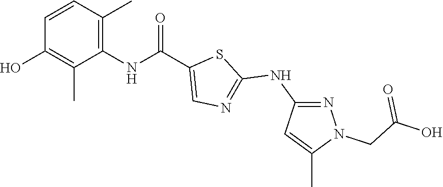 [SMILES: Cc1ccc(O)c(C)c1NC(=O)c1cnc(Nc2cc(C)n(CC(=O)O)n2)s1] |
| BAA- 125 | 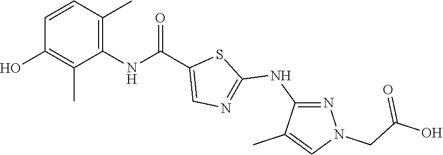 [SMILES: Cc1cn(CC(=O)O)nc1Nc1ncc(C(=O)Nc2c(C)ccc(O)c2C)s1] |
| BAA- 126 | 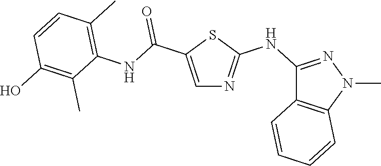 [SMILES: Cc1ccc(O)c(C)c1NC(=O)c1cnc(Nc2nn(C)c3ccccc23)s1] |
| BAA- 127 | 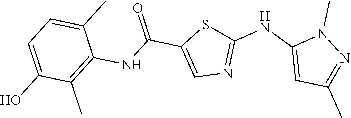 [SMILES: Cc1cc(Nc2ncc(C(=O)Nc3c(C)ccc(O)c3C)s2)n(C)n1] |
| BAA- 128 | 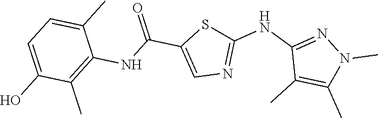 [SMILES: Cc1ccc(O)c(C)c1NC(=O)c1cnc(Nc2nn(C)c(C)c2C)s1] |
| BAA- 129 | 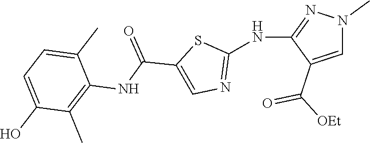 [SMILES: CCOC(=O)c1cn(C)nc1Nc1ncc(C(=O)Nc2c(C)ccc(O)c2C)s1] |
| BAA- 130 | 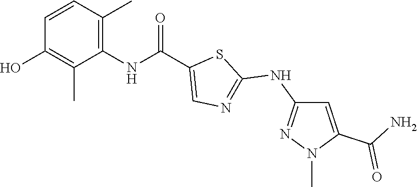 [SMILES: Cc1ccc(O)c(C)c1NC(=O)c1cnc(Nc2cc(C(N)=O)n(C)n2)s1] |
| BAA- 131 | 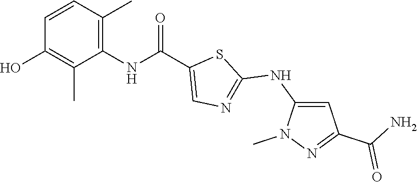 [SMILES: Cc1ccc(O)c(C)c1NC(=O)c1cnc(Nc2cc(C(N)=O)nn2C)s1] |
| BAA- 132 | 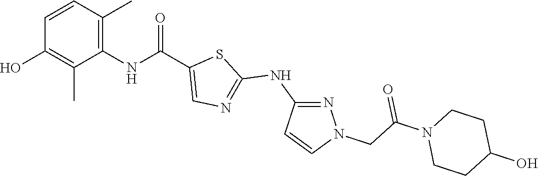 [SMILES: Cc1ccc(O)c(C)c1NC(=O)c1cnc(Nc2ccn(CC(=O)N3CCC(O)CC3)n2)s1] |
| BAA- 133 | 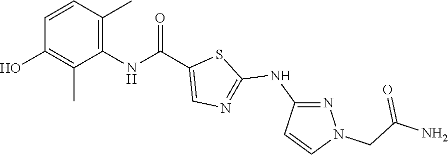 [SMILES: Cc1ccc(O)c(C)c1NC(=O)c1cnc(Nc2ccn(CC(N)=O)n2)s1] |
| BAA- 134 | 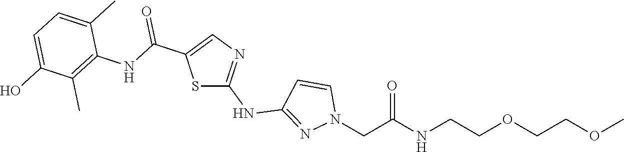 [SMILES: COCCOCCNC(=O)Cn1ccc(Nc2ncc(C(=O)Nc3c(C)ccc(O)c3C)s2)n1] |
| BAA- 135 | 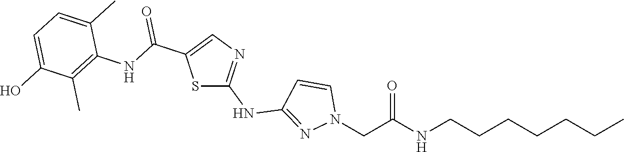 [SMILES: CCCCCCCNC(=O)Cn1ccc(Nc2ncc(C(=O)Nc3c(C)ccc(O)c3C)s2)n1] |
| BAA- 136 | 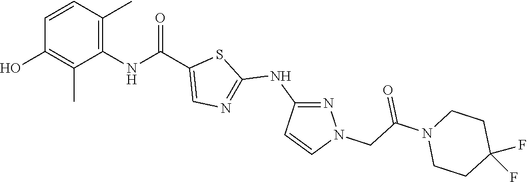 [SMILES: Cc1ccc(O)c(C)c1NC(=O)c1cnc(Nc2ccn(CC(=O)N3CCC(F)(F)CC3)n2)s1] |
| BAA- 137 | 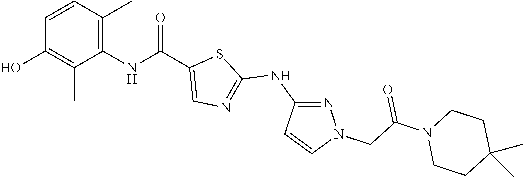 [SMILES: Cc1ccc(O)c(C)c1NC(=O)c1cnc(Nc2ccn(CC(=O)N3CCC(C)(C)CC3)n2)s1] |
| BAA- 138 | 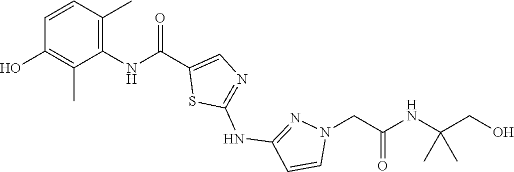 [SMILES: Cc1ccc(O)c(C)c1NC(=O)c1cnc(Nc2ccn(CC(=O)NC(C)(C)CO)n2)s1] |
| BAA- 139 | 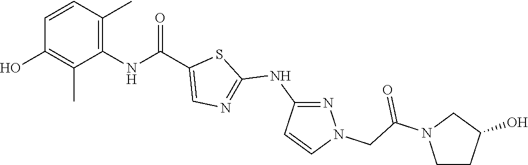 [SMILES: Cc1ccc(O)c(C)c1NC(=O)c1cnc(Nc2ccn(CC(=O)N3CC[C@@H](O)C3)n2)s1] |
| BAA- 140 | 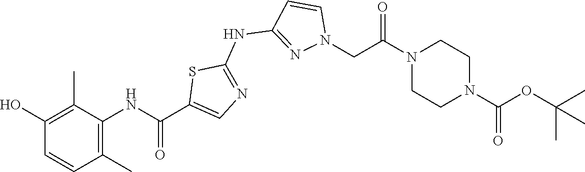 [SMILES: Cc1ccc(O)c(C)c1NC(=O)c1cnc(Nc2ccn(CC(=O)N3CCN(C(=O)OC(C)(C)C)CC3)n2)s1] |
| BAA- 141 | 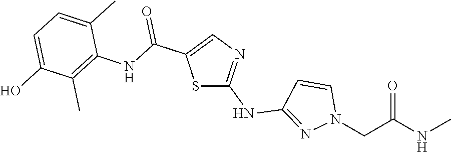 [SMILES: CNC(=O)Cn1ccc(Nc2ncc(C(=O)Nc3c(C)ccc(O)c3C)s2)n1] |
| BAA- 142 | 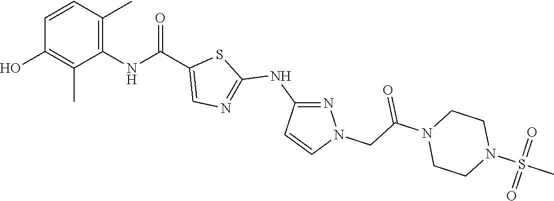 [SMILES: Cc1ccc(O)c(C)c1NC(=O)c1cnc(Nc2ccn(CC(=O)N3CCN(S(C)(=O)=O)CC3)n2)s1] |
| BAA- 143 | 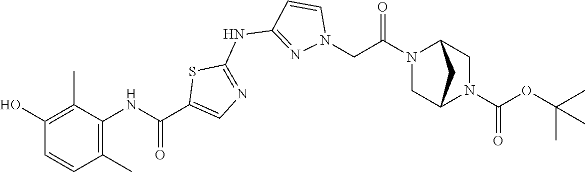 [SMILES: Cc1ccc(O)c(C)c1NC(=O)c1cnc(Nc2ccn(CC(=O)N3C[C@@H]4C[C@H]3CN4C(=O)OC(C)(C)C)n2)s1] |
| BAA- 144 | 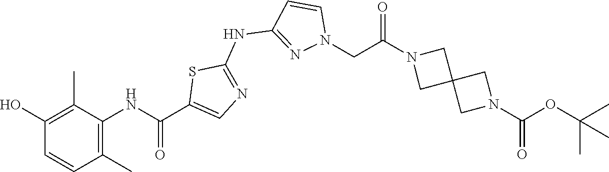 [SMILES: Cc1ccc(O)c(C)c1NC(=O)c1cnc(Nc2ccn(CC(=O)N3CC4(C3)CN(C(=O)OC(C)(C)C)C4)n2)s1] |
| BAA- 145 | 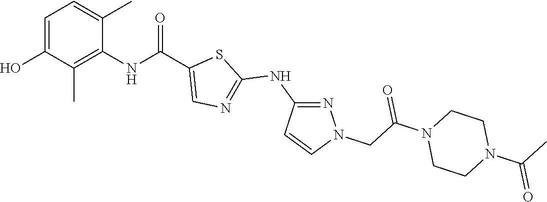 [SMILES: CC(=O)N1CCN(C(=O)Cn2ccc(Nc3ncc(C(=O)Nc4c(C)ccc(O)c4C)s3)n2)CC1] |
| BAA- 146 | 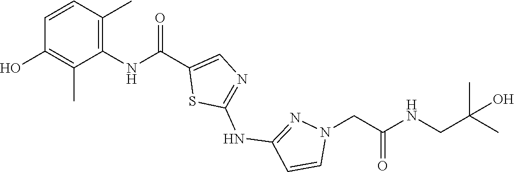 [SMILES: Cc1ccc(O)c(C)c1NC(=O)c1cnc(Nc2ccn(CC(=O)NCC(C)(C)O)n2)s1] |
| BAA- 147 | 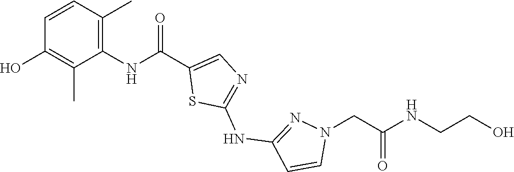 [SMILES: Cc1ccc(O)c(C)c1NC(=O)c1cnc(Nc2ccn(CC(=O)NCCO)n2)s1] |
| BAA- 148 | 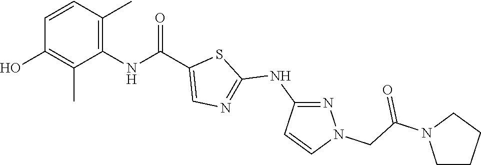 [SMILES: Cc1ccc(O)c(C)c1NC(=O)c1cnc(Nc2ccn(CC(=O)N3CCCC3)n2)s1] |
| BAA- 149 | 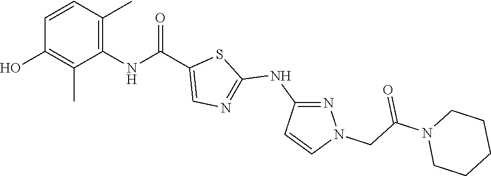 [SMILES: Cc1ccc(O)c(C)c1NC(=O)c1cnc(Nc2ccn(CC(=O)N3CCCCC3)n2)s1] |
| BAA- 150 | 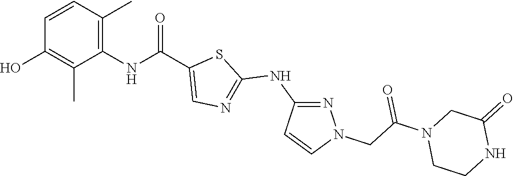 [SMILES: Cc1ccc(O)c(C)c1NC(=O)c1cnc(Nc2ccn(CC(=O)N3CCNC(=O)C3)n2)s1] |
| BAA- 151 | 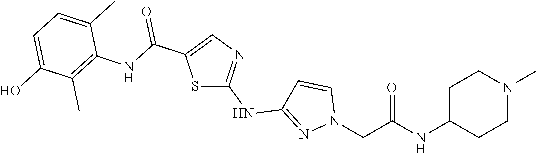 [SMILES: Cc1ccc(O)c(C)c1NC(=O)c1cnc(Nc2ccn(CC(=O)NC3CCN(C)CC3)n2)s1] |
| BAA- 152 | 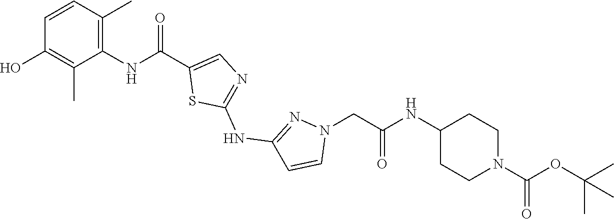 [SMILES: Cc1ccc(O)c(C)c1NC(=O)c1cnc(Nc2ccn(CC(=O)NC3CCN(C(=O)OC(C)(C)C)CC3)n2)s1] |
| BAA- 153 | 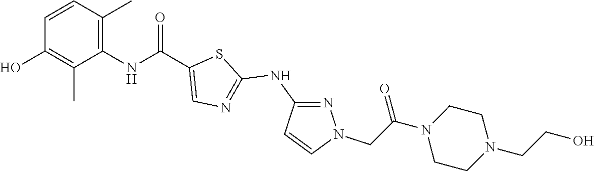 [SMILES: Cc1ccc(O)c(C)c1NC(=O)c1cnc(Nc2ccn(CC(=O)N3CCN(CCO)CC3)n2)s1] |
| BAA- 154 | 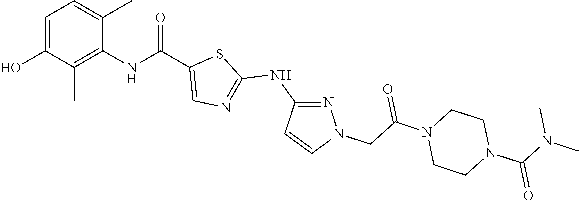 [SMILES: Cc1ccc(O)c(C)c1NC(=O)c1cnc(Nc2ccn(CC(=O)N3CCN(C(=O)N(C)C)CC3)n2)s1] |
| BAA- 155 | 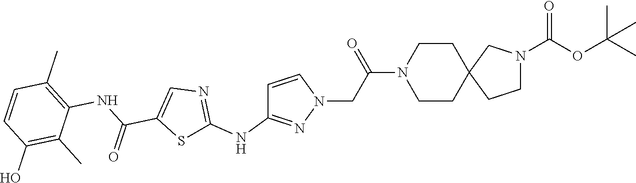 [SMILES: Cc1ccc(O)c(C)c1NC(=O)c1cnc(Nc2ccn(CC(=O)N3CCC4(CC3)CCN(C(=O)OC(C)(C)C)C4)n2)s1] |
| BAA- 156 | 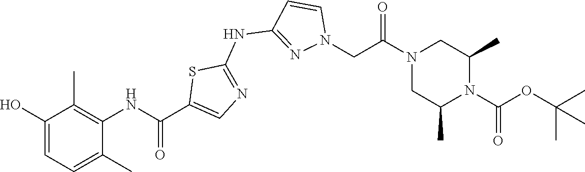 [SMILES: Cc1ccc(O)c(C)c1NC(=O)c1cnc(Nc2ccn(CC(=O)N3C[C@@H](C)N(C(=O)OC(C)(C)C)[C@@H](C)C3)n2)s1] |
| BAA- 157 | 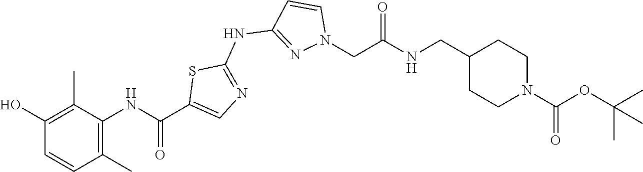 [SMILES: Cc1ccc(O)c(C)c1NC(=O)c1cnc(Nc2ccn(CC(=O)NCC3CCN(C(=O)OC(C)(C)C)CC3)n2)s1] |
| BAA- 158 | 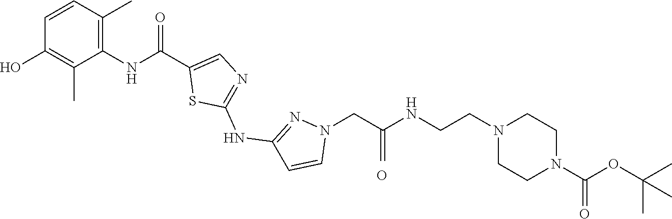 [SMILES: Cc1ccc(O)c(C)c1NC(=O)c1cnc(Nc2ccn(CC(=O)NCCN3CCN(C(=O)OC(C)(C)C)CC3)n2)s1] |
| BAA- 159 | 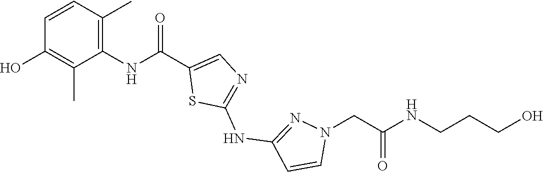 [SMILES: Cc1ccc(O)c(C)c1NC(=O)c1cnc(Nc2ccn(CC(=O)NCCCO)n2)s1] |
| BAA- 160 | 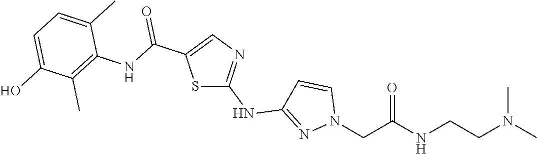 [SMILES: Cc1ccc(O)c(C)c1NC(=O)c1cnc(Nc2ccn(CC(=O)NCCN(C)C)n2)s1] |
| BAA- 161 | 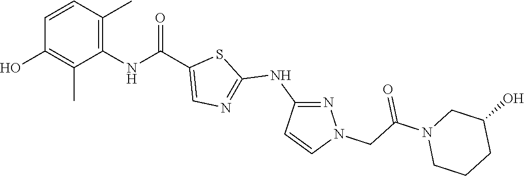 [SMILES: Cc1ccc(O)c(C)c1NC(=O)c1cnc(Nc2ccn(CC(=O)N3CCC[C@@H](O)C3)n2)s1] |
| BAA- 162 | 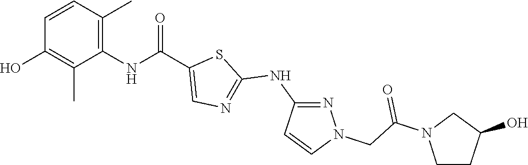 [SMILES: Cc1ccc(O)c(C)c1NC(=O)c1cnc(Nc2ccn(CC(=O)N3CC[C@H](O)C3)n2)s1] |
| BAA- 163 | 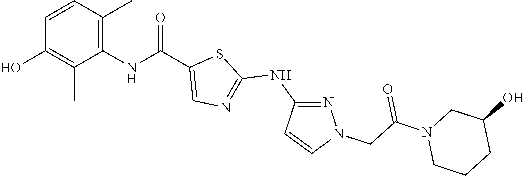 [SMILES: Cc1ccc(O)c(C)c1NC(=O)c1cnc(Nc2ccn(CC(=O)N3CCC[C@H](O)C3)n2)s1] |
| BAA- 164 | 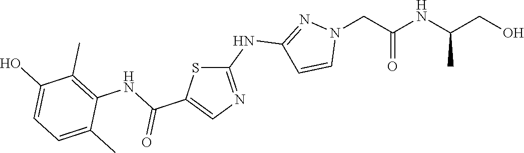 [SMILES: Cc1ccc(O)c(C)c1NC(=O)c1cnc(Nc2ccn(CC(=O)N[C@H](C)CO)n2)s1] |
| BAA- 165 | 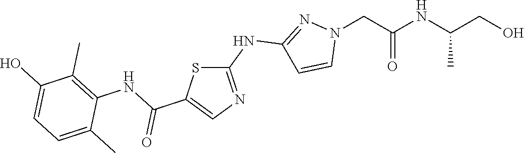 [SMILES: Cc1ccc(O)c(C)c1NC(=O)c1cnc(Nc2ccn(CC(=O)N[C@@H](C)CO)n2)s1] |
| BAA- 166 | 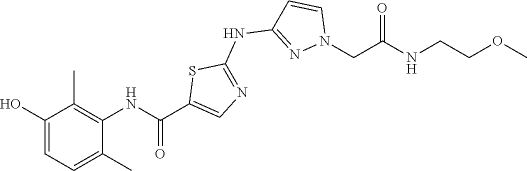 [SMILES: COCCNC(=O)Cn1ccc(Nc2ncc(C(=O)Nc3c(C)ccc(O)c3C)s2)n1] |
| BAA- 167 | 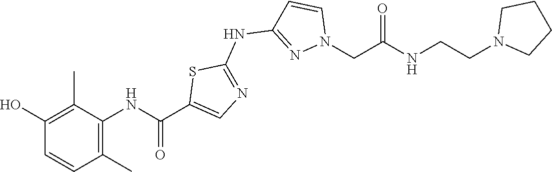 [SMILES: Cc1ccc(O)c(C)c1NC(=O)c1cnc(Nc2ccn(CC(=O)NCCN3CCCC3)n2)s1] |
| BAA- 168 | 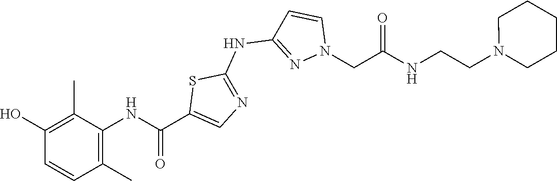 [SMILES: Cc1ccc(O)c(C)c1NC(=O)c1cnc(Nc2ccn(CC(=O)NCCN3CCCCC3)n2)s1] |
| BAA- 169 | 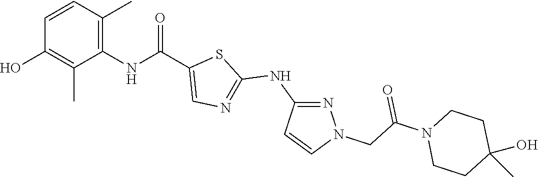 [SMILES: Cc1ccc(O)c(C)c1NC(=O)c1cnc(Nc2ccn(CC(=O)N3CCC(C)(O)CC3)n2)s1] |
| BAA- 170 | 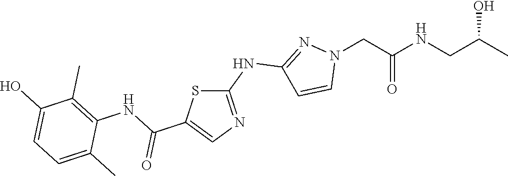 [SMILES: Cc1ccc(O)c(C)c1NC(=O)c1cnc(Nc2ccn(CC(=O)NC[C@@H](C)O)n2)s1] |
| BAA- 171 | 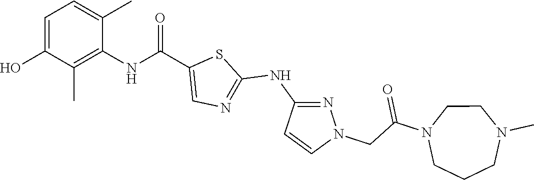 [SMILES: Cc1ccc(O)c(C)c1NC(=O)c1cnc(Nc2ccn(CC(=O)N3CCCN(C)CC3)n2)s1] |
| BAA- 172 | 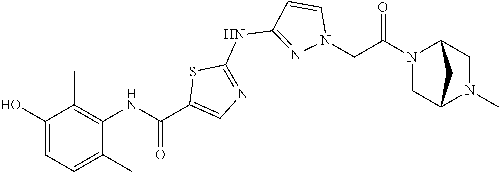 [SMILES: Cc1ccc(O)c(C)c1NC(=O)c1cnc(Nc2ccn(CC(=O)N3C[C@@H]4C[C@H]3CN4C)n2)s1] |
| BAA- 173 | 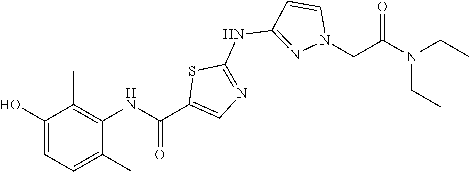 [SMILES: CCN(CC)C(=O)Cn1ccc(Nc2ncc(C(=O)Nc3c(C)ccc(O)c3C)s2)n1] |
| BAA- 174 | 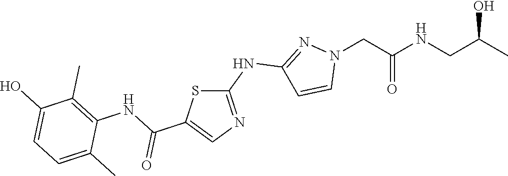 [SMILES: Cc1ccc(O)c(C)c1NC(=O)c1cnc(Nc2ccn(CC(=O)NC[C@H](C)O)n2)s1] |
| BAA- 175 | 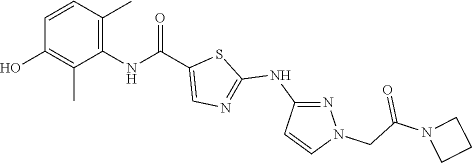 [SMILES: Cc1ccc(O)c(C)c1NC(=O)c1cnc(Nc2ccn(CC(=O)N3CCC3)n2)s1] |
| BAA- 176 | 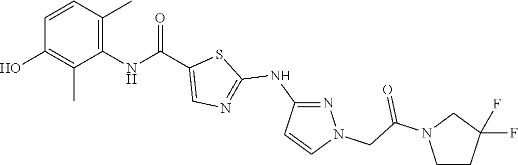 [SMILES: Cc1ccc(O)c(C)c1NC(=O)c1cnc(Nc2ccn(CC(=O)N3CCC(F)(F)C3)n2)s1] |
| BAA- 177 | 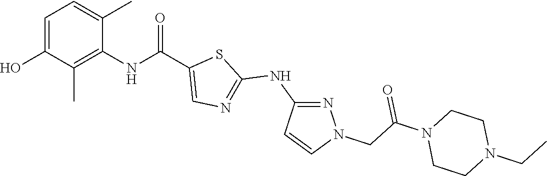 [SMILES: CCN1CCN(C(=O)Cn2ccc(Nc3ncc(C(=O)Nc4c(C)ccc(O)c4C)s3)n2)CC1] |
| BAA- 178 | 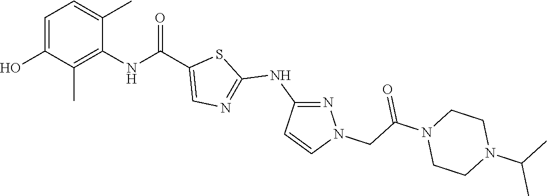 [SMILES: Cc1ccc(O)c(C)c1NC(=O)c1cnc(Nc2ccn(CC(=O)N3CCN(C(C)C)CC3)n2)s1] |
| BAA- 179 | 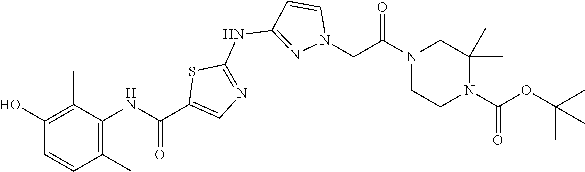 [SMILES: Cc1ccc(O)c(C)c1NC(=O)c1cnc(Nc2ccn(CC(=O)N3CCN(C(=O)OC(C)(C)C)C(C)(C)C3)n2)s1] |
| BAA- 180 | 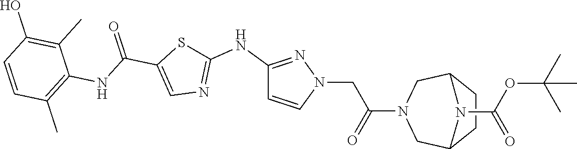 [SMILES: Cc1ccc(O)c(C)c1NC(=O)c1cnc(Nc2ccn(CC(=O)N3CC4CCC(C3)N4C(=O)OC(C)(C)C)n2)s1] |
| BAA- 181 | 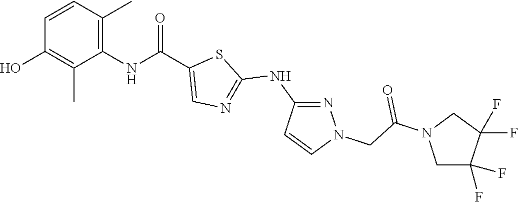 [SMILES: Cc1ccc(O)c(C)c1NC(=O)c1cnc(Nc2ccn(CC(=O)N3CC(F)(F)C(F)(F)C3)n2)s1] |
| BAA- 182 | 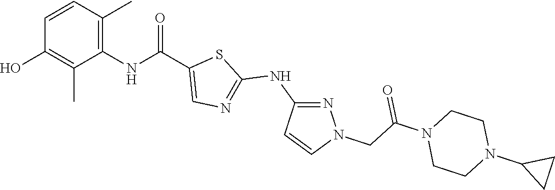 [SMILES: Cc1ccc(O)c(C)c1NC(=O)c1cnc(Nc2ccn(CC(=O)N3CCN(C4CC4)CC3)n2)s1] |
| BAA- 183 | 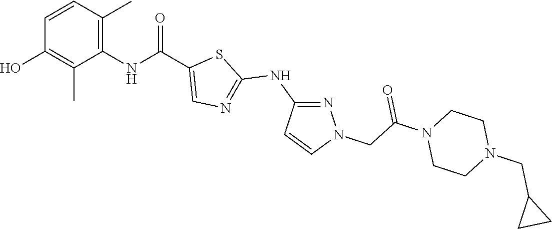 [SMILES: Cc1ccc(O)c(C)c1NC(=O)c1cnc(Nc2ccn(CC(=O)N3CCN(CC4CC4)CC3)n2)s1] |
| BAA- 184 | 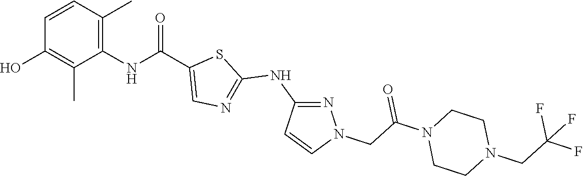 [SMILES: Cc1ccc(O)c(C)c1NC(=O)c1cnc(Nc2ccn(CC(=O)N3CCN(CC(F)(F)F)CC3)n2)s1] |
| BAA- 185 | 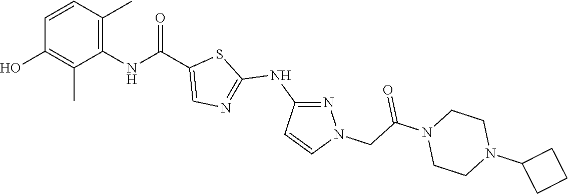 [SMILES: Cc1ccc(O)c(C)c1NC(=O)c1cnc(Nc2ccn(CC(=O)N3CCN(C4CCC4)CC3)n2)s1] |
| BAA- 186 | 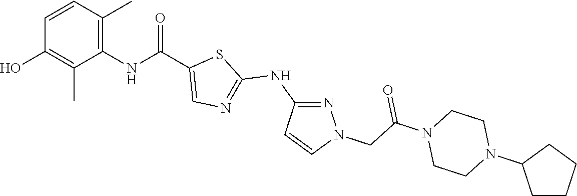 [SMILES: Cc1ccc(O)c(C)c1NC(=O)c1cnc(Nc2ccn(CC(=O)N3CCN(C4CCCC4)CC3)n2)s1] |
| BAA- 187 | 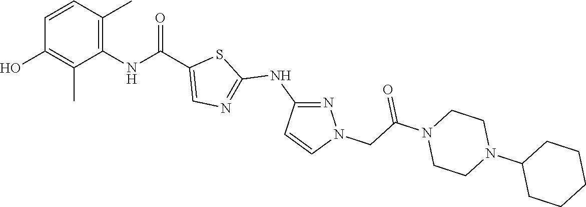 [SMILES: Cc1ccc(O)c(C)c1NC(=O)c1cnc(Nc2ccn(CC(=O)N3CCN(C4CCCCC4)CC3)n2)s1] |
| BAA- 188 | 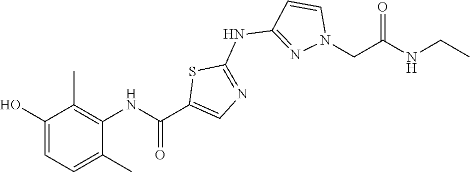 [SMILES: CCNC(=O)Cn1ccc(Nc2ncc(C(=O)Nc3c(C)ccc(O)c3C)s2)n1] |
| BAA- 189 | 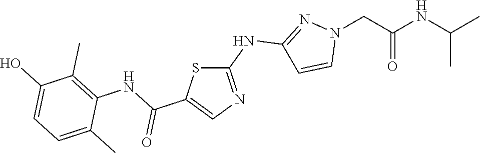 [SMILES: Cc1ccc(O)c(C)c1NC(=O)c1cnc(Nc2ccn(CC(=O)NC(C)C)n2)s1] |
| BAA- 190 | 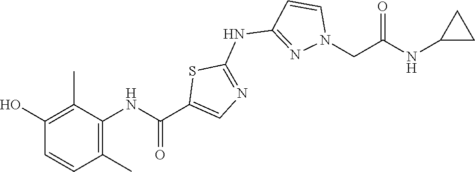 [SMILES: Cc1ccc(O)c(C)c1NC(=O)c1cnc(Nc2ccn(CC(=O)NC3CC3)n2)s1] |
| BAA- 191 | 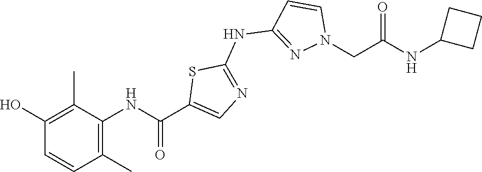 [SMILES: Cc1ccc(O)c(C)c1NC(=O)c1cnc(Nc2ccn(CC(=O)NC3CCC3)n2)s1] |
| BAA- 192 | 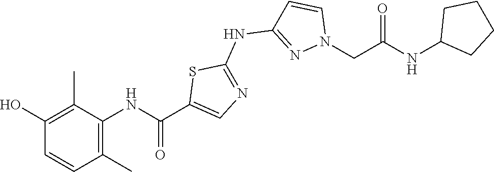 [SMILES: Cc1ccc(O)c(C)c1NC(=O)c1cnc(Nc2ccn(CC(=O)NC3CCCC3)n2)s1] |
| BAA- 193 | 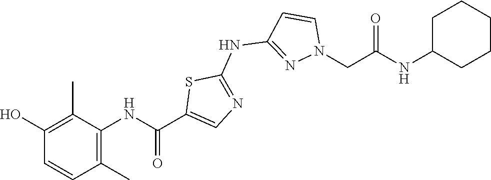 [SMILES: Cc1ccc(O)c(C)c1NC(=O)c1cnc(Nc2ccn(CC(=O)NC3CCCCC3)n2)s1] |
| BAA- 194 | 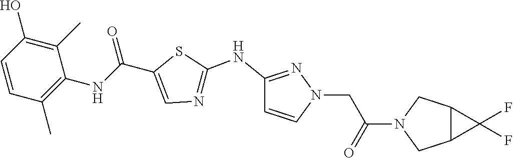 [SMILES: Cc1ccc(O)c(C)c1NC(=O)c1cnc(Nc2ccn(CC(=O)N3CC4C(C3)C4(F)F)n2)s1] |
| BAA- 195 | 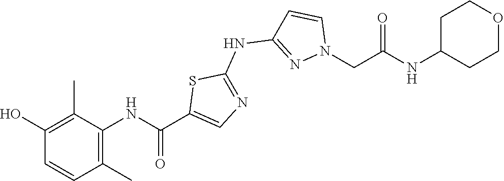 [SMILES: Cc1ccc(O)c(C)c1NC(=O)c1cnc(Nc2ccn(CC(=O)NC3CCOCC3)n2)s1] |
| BAA- 196 | 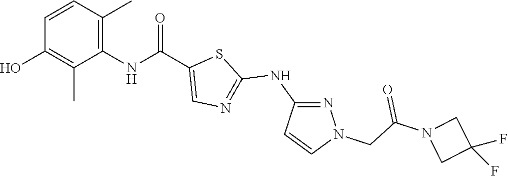 [SMILES: Cc1ccc(O)c(C)c1NC(=O)c1cnc(Nc2ccn(CC(=O)N3CC(F)(F)C3)n2)s1] |
| BAA- 197 | 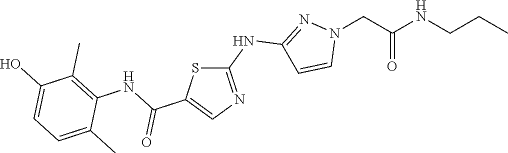 [SMILES: CCCNC(=O)Cn1ccc(Nc2ncc(C(=O)Nc3c(C)ccc(O)c3C)s2)n1] |
| BAA- 198 | 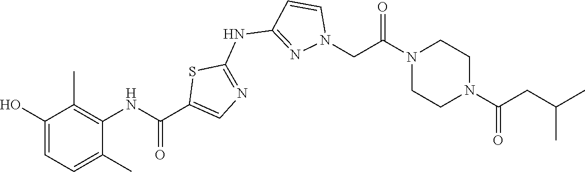 [SMILES: Cc1ccc(O)c(C)c1NC(=O)c1cnc(Nc2ccn(CC(=O)N3CCN(C(=O)CC(C)C)CC3)n2)s1] |
| BAA- 199 | 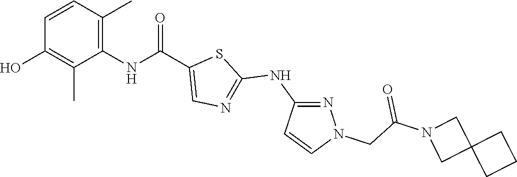 [SMILES: Cc1ccc(O)c(C)c1NC(=O)c1cnc(Nc2ccn(CC(=O)N3CC4(CCC4)C3)n2)s1] |
| BAA- 200 | 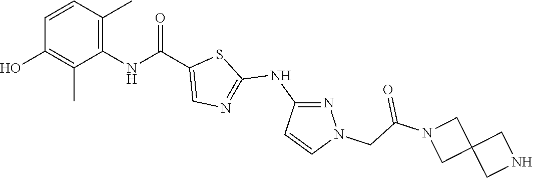 [SMILES: Cc1ccc(O)c(C)c1NC(=O)c1cnc(Nc2ccn(CC(=O)N3CC4(CNC4)C3)n2)s1] |
| BAA- 201 | 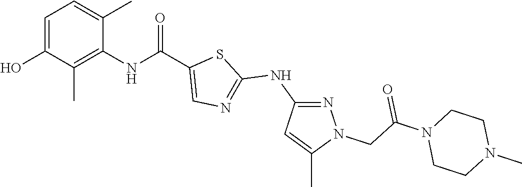 [SMILES: Cc1ccc(O)c(C)c1NC(=O)c1cnc(Nc2cc(C)n(CC(=O)N3CCN(C)CC3)n2)s1] |
| BAA- 202 | 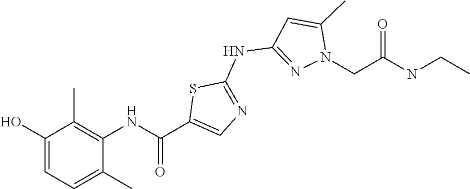 [SMILES: CCNC(=O)Cn1nc(Nc2ncc(C(=O)Nc3c(C)ccc(O)c3C)s2)cc1C] |
| BAA- 203 | 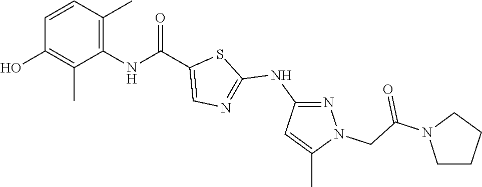 [SMILES: Cc1ccc(O)c(C)c1NC(=O)c1cnc(Nc2cc(C)n(CC(=O)N3CCCC3)n2)s1] |
| BAA- 204 | 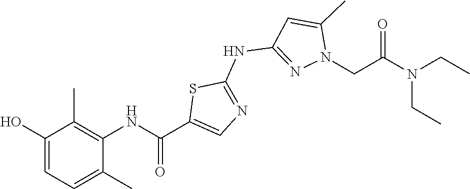 [SMILES: CCN(CC)C(=O)Cn1nc(Nc2ncc(C(=O)Nc3c(C)ccc(O)c3C)s2)cc1C] |
| BAA- 205 | 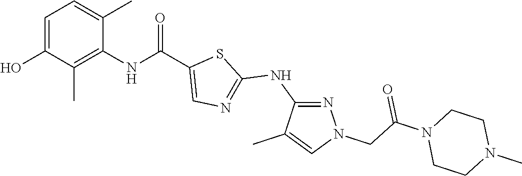 [SMILES: Cc1cn(CC(=O)N2CCN(C)CC2)nc1Nc1ncc(C(=O)Nc2c(C)ccc(O)c2C)s1] |
| BAA- 206 | 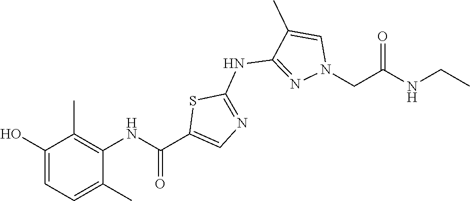 [SMILES: CCNC(=O)Cn1cc(C)c(Nc2ncc(C(=O)Nc3c(C)ccc(O)c3C)s2)n1] |
| BAA- 207 | 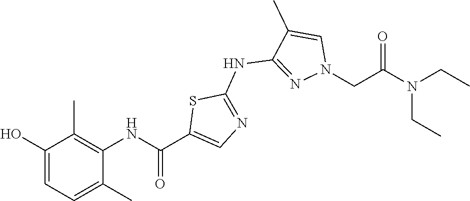 [SMILES: CCN(CC)C(=O)Cn1cc(C)c(Nc2ncc(C(=O)Nc3c(C)ccc(O)c3C)s2)n1] |
| BAA- 208 | 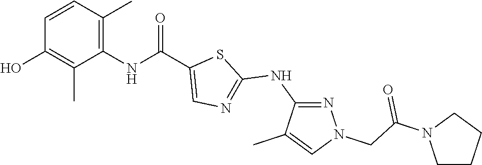 [SMILES: Cc1cn(CC(=O)N2CCCC2)nc1Nc1ncc(C(=O)Nc2c(C)ccc(O)c2C)s1] |
| BAA- 209 | 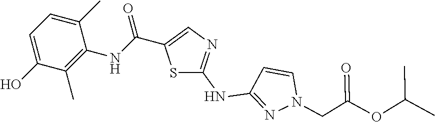 [SMILES: Cc1ccc(O)c(C)c1NC(=O)c1cnc(Nc2ccn(CC(=O)OC(C)C)n2)s1] |
| BAA- 210 | 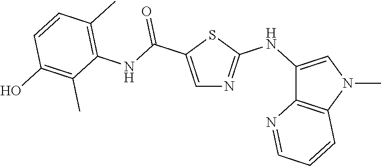 [SMILES: Cc1ccc(O)c(C)c1NC(=O)c1cnc(Nc2cn(C)c3cccnc23)s1] |
| BAA- 211 | 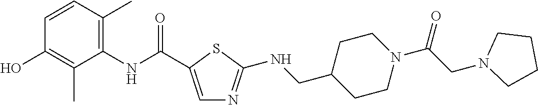 [SMILES: Cc1ccc(O)c(C)c1NC(=O)c1cnc(NCC2CCN(C(=O)CN3CCCC3)CC2)s1] |
| BAA- 212 | 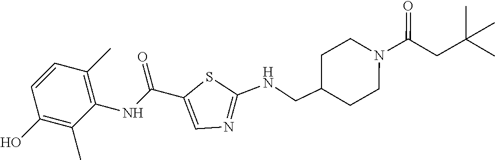 [SMILES: Cc1ccc(O)c(C)c1NC(=O)c1cnc(NCC2CCN(C(=O)CC(C)(C)C)CC2)s1] |
| BAA- 213 | 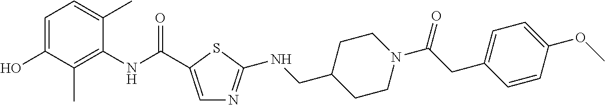 [SMILES: COc1ccc(CC(=O)N2CCC(CNc3ncc(C(=O)Nc4c(C)ccc(O)c4C)s3)CC2)cc1] |
| BAA- 214 | 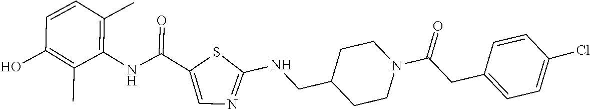 [SMILES: Cc1ccc(O)c(C)c1NC(=O)c1cnc(NCC2CCN(C(=O)Cc3ccc(Cl)cc3)CC2)s1] |
| BAA- 215 | 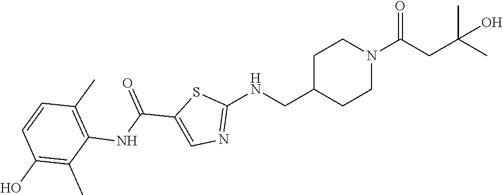 [SMILES: Cc1ccc(O)c(C)c1NC(=O)c1cnc(NCC2CCN(C(=O)CC(C)(C)O)CC2)s1] |
| BAA- 216 | 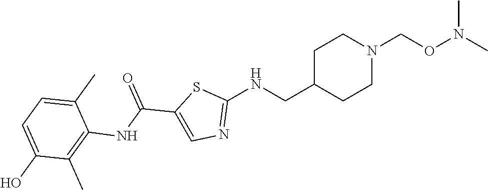 [SMILES: Cc1ccc(O)c(C)c1NC(=O)c1cnc(NCC2CCN(CON(C)C)CC2)s1] |
| BAA- 217 | 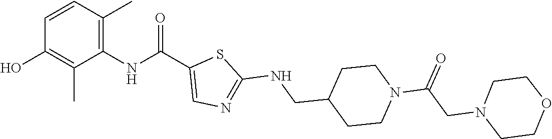 [SMILES: Cc1ccc(O)c(C)c1NC(=O)c1cnc(NCC2CCN(C(=O)CN3CCOCC3)CC2)s1] |
| BAA- 218 | 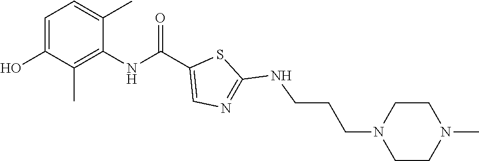 [SMILES: Cc1ccc(O)c(C)c1NC(=O)c1cnc(NCCCN2CCN(C)CC2)s1] |
| BAA- 219 | 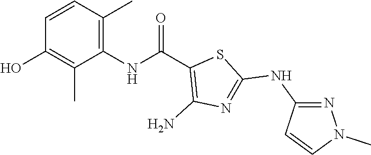 [SMILES: Cc1ccc(O)c(C)c1NC(=O)c1sc(Nc2ccn(C)n2)nc1N] |
| BAA- 220 | 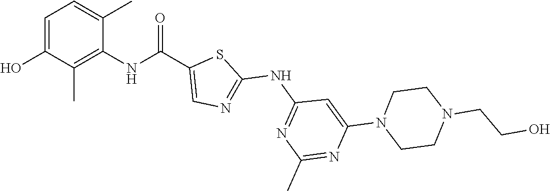 [SMILES: Cc1nc(Nc2ncc(C(=O)Nc3c(C)ccc(O)c3C)s2)cc(N2CCN(CCO)CC2)n1] |
| BAA- 221 | 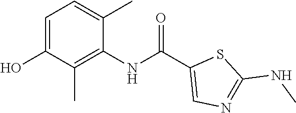 [SMILES: CNc1ncc(C(=O)Nc2c(C)ccc(O)c2C)s1] |
| BAA- 222 | 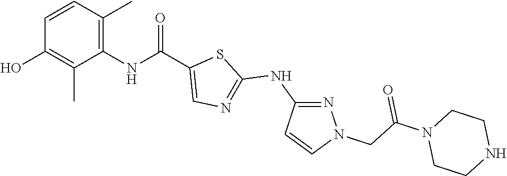 [SMILES: Cc1ccc(O)c(C)c1NC(=O)c1cnc(Nc2ccn(CC(=O)N3CCNCC3)n2)s1] |
| BAA- 223 | 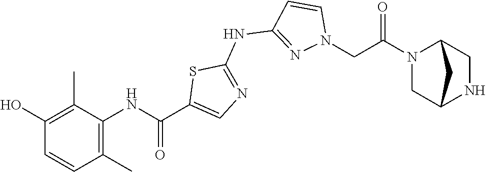 [SMILES: Cc1ccc(O)c(C)c1NC(=O)c1cnc(Nc2ccn(CC(=O)N3C[C@@H]4C[C@H]3CN4)n2)s1] |
| BAA- 224 | 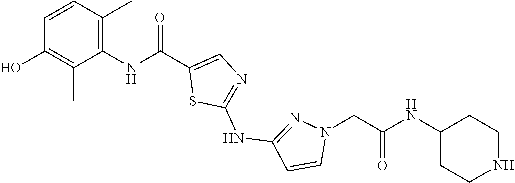 [SMILES: Cc1ccc(O)c(C)c1NC(=O)c1cnc(Nc2ccn(CC(=O)NC3CCNCC3)n2)s1] |
| BAA- 225 | 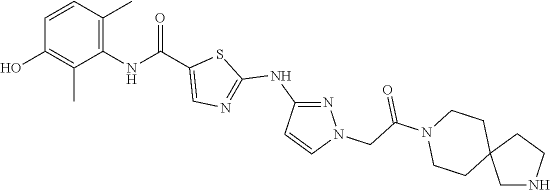 [SMILES: Cc1ccc(O)c(C)c1NC(=O)c1cnc(Nc2ccn(CC(=O)N3CCC4(CCNC4)CC3)n2)s1] |
| BAA- 226 | 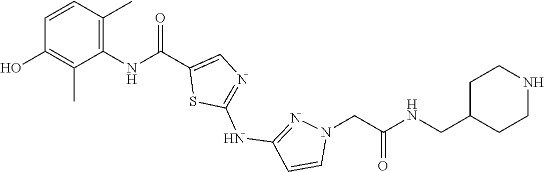 [SMILES: Cc1ccc(O)c(C)c1NC(=O)c1cnc(Nc2ccn(CC(=O)NCC3CCNCC3)n2)s1] |
| BAA- 227 | 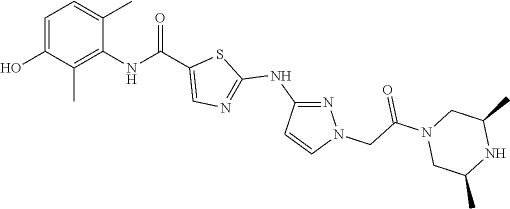 [SMILES: Cc1ccc(O)c(C)c1NC(=O)c1cnc(Nc2ccn(CC(=O)N3C[C@@H](C)N[C@@H](C)C3)n2)s1] |
| BAA- 228 | 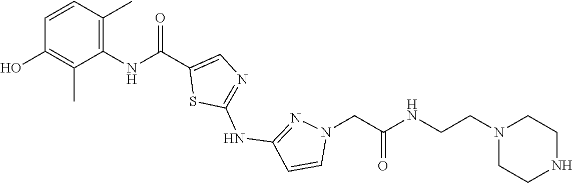 [SMILES: Cc1ccc(O)c(C)c1NC(=O)c1cnc(Nc2ccn(CC(=O)NCCN3CCNCC3)n2)s1] |
| BAA- 229 | 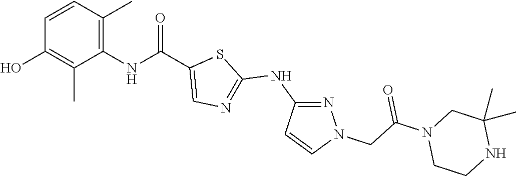 [SMILES: Cc1ccc(O)c(C)c1NC(=O)c1cnc(Nc2ccn(CC(=O)N3CCNC(C)(C)C3)n2)s1] |
| BAA- 230 | 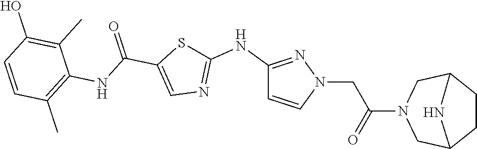 [SMILES: Cc1ccc(O)c(C)c1NC(=O)c1cnc(Nc2ccn(CC(=O)N3CC4CCC(C3)N4)n2)s1] |
| BAA- 231 | 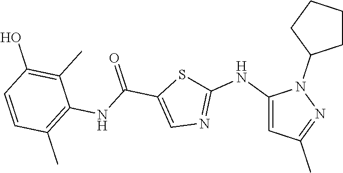 [SMILES: Cc1cc(Nc2ncc(C(=O)Nc3c(C)ccc(O)c3C)s2)n(C2CCCC2)n1] |
| BAA- 232 | 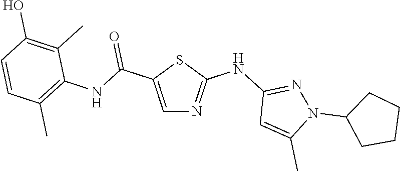 [SMILES: Cc1ccc(O)c(C)c1NC(=O)c1cnc(Nc2cc(C)n(C3CCCC3)n2)s1] |
| BAA- 233 | 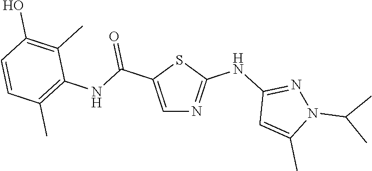 [SMILES: Cc1ccc(O)c(C)c1NC(=O)c1cnc(Nc2cc(C)n(C(C)C)n2)s1] |
| BAA- 234 | 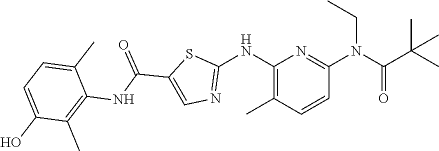 [SMILES: CCN(C(=O)C(C)(C)C)c1ccc(C)c(Nc2ncc(C(=O)Nc3c(C)ccc(O)c3C)s2)n1] |
| BAA- 235 | 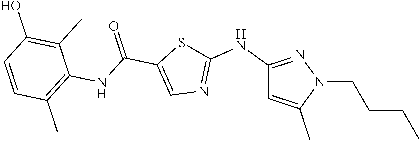 [SMILES: CCCCn1nc(Nc2ncc(C(=O)Nc3c(C)ccc(O)c3C)s2)cc1C] |
| BAA- 236 | 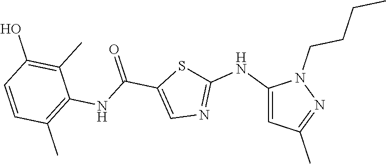 [SMILES: CCCCn1nc(C)cc1Nc1ncc(C(=O)Nc2c(C)ccc(O)c2C)s1] |
| BAA- 237 | 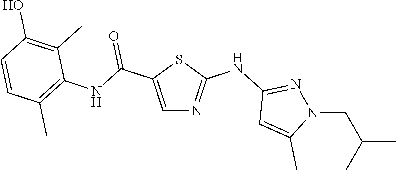 [SMILES: Cc1ccc(O)c(C)c1NC(=O)c1cnc(Nc2cc(C)n(CC(C)C)n2)s1] |
| BAA- 238 | 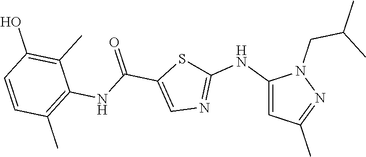 [SMILES: Cc1cc(Nc2ncc(C(=O)Nc3c(C)ccc(O)c3C)s2)n(CC(C)C)n1] |
| BAA- 239 | 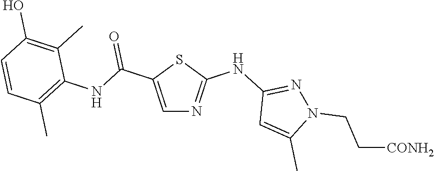 [SMILES: Cc1ccc(O)c(C)c1NC(=O)c1cnc(Nc2cc(C)n(CCC(N)=O)n2)s1] |
| BAA- 240 | 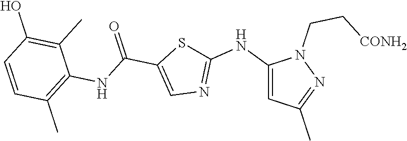 [SMILES: Cc1cc(Nc2ncc(C(=O)Nc3c(C)ccc(O)c3C)s2)n(CCC(N)=O)n1] |
| BAA- 241 | 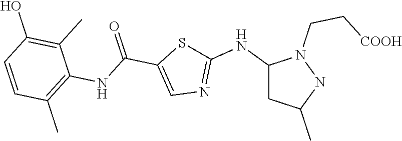 [SMILES: Cc1ccc(O)c(C)c1NC(=O)c1cnc(NC2CC(C)NN2CCC(=O)O)s1] |
| BAA- 242 | 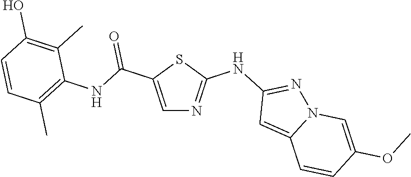 [SMILES: COc1ccc2cc(Nc3ncc(C(=O)Nc4c(C)ccc(O)c4C)s3)nn2c1] |
| BAA- 243 | 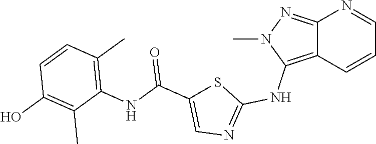 [SMILES: Cc1ccc(O)c(C)c1NC(=O)c1cnc(Nc2c3cccnc3nn2C)s1] |
| BAA- 244 | 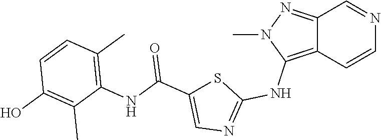 [SMILES: Cc1ccc(O)c(C)c1NC(=O)c1cnc(Nc2c3ccncc3nn2C)s1] |
| BAA- 245 | 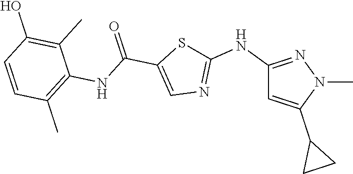 [SMILES: Cc1ccc(O)c(C)c1NC(=O)c1cnc(Nc2cc(C3CC3)n(C)n2)s1] |
| BAA- 246 | 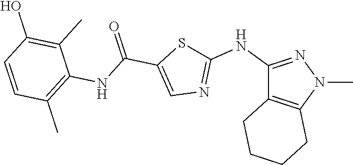 [SMILES: Cc1ccc(O)c(C)c1NC(=O)c1cnc(Nc2nn(C)c3c2CCCC3)s1] |
| BAA- 247 | 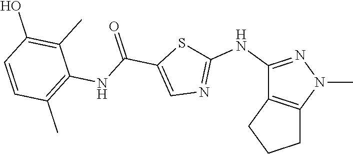 [SMILES: Cc1ccc(O)c(C)c1NC(=O)c1cnc(Nc2nn(C)c3c2CCC3)s1] |
| BAA- 248 | 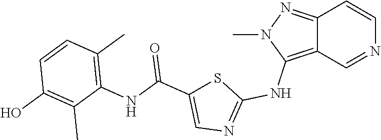 [SMILES: Cc1ccc(O)c(C)c1NC(=O)c1cnc(Nc2c3cnccc3nn2C)s1] |
| BAA- 249 | 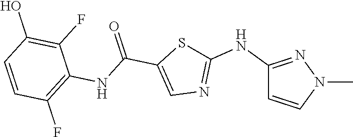 [SMILES: Cn1ccc(Nc2ncc(C(=O)Nc3c(F)ccc(O)c3F)s2)n1] |
| BAA- 250 | 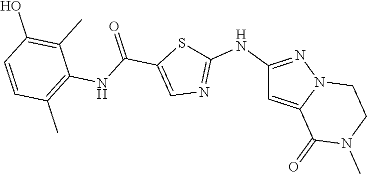 [SMILES: Cc1ccc(O)c(C)c1NC(=O)c1cnc(Nc2cc3n(n2)CCN(C)C3=O)s1] |
| BAA- 251 | 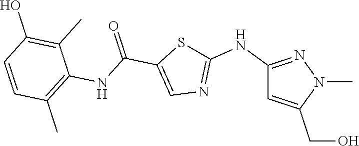 [SMILES: Cc1ccc(O)c(C)c1NC(=O)c1cnc(Nc2cc(CO)n(C)n2)s1] |
| BAA- 252 | 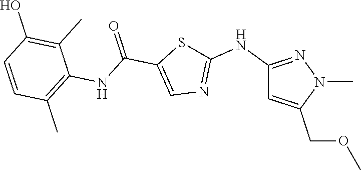 [SMILES: COCc1cc(Nc2ncc(C(=O)Nc3c(C)ccc(O)c3C)s2)nn1C] |
| BAA- 253 | 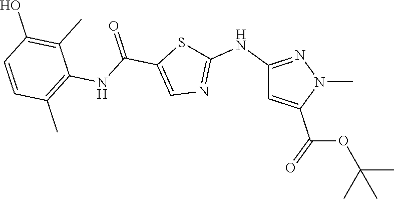 [SMILES: Cc1ccc(O)c(C)c1NC(=O)c1cnc(Nc2cc(C(=O)OC(C)(C)C)n(C)n2)s1] |
| BAA- 254 | 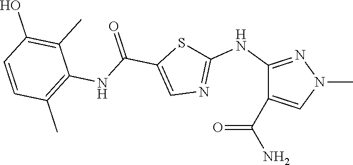 [SMILES: Cc1ccc(O)c(C)c1NC(=O)c1cnc(Nc2nn(C)cc2C(N)=O)s1] |
| BAA- 255 | 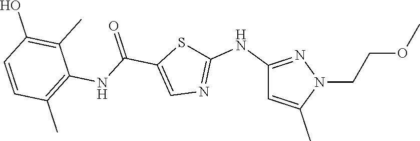 [SMILES: COCCn1nc(Nc2ncc(C(=O)Nc3c(C)ccc(O)c3C)s2)cc1C] |
| BAA- 256 | 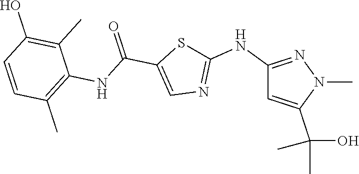 [SMILES: Cc1ccc(O)c(C)c1NC(=O)c1cnc(Nc2cc(C(C)(C)O)n(C)n2)s1] |
| BAA- 257 | 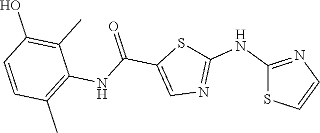 [SMILES: Cc1ccc(O)c(C)c1NC(=O)c1cnc(Nc2nccs2)s1] |
| BAA- 258 | 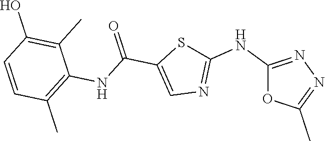 [SMILES: Cc1nnc(Nc2ncc(C(=O)Nc3c(C)ccc(O)c3C)s2)o1] |
| BAA- 259 | 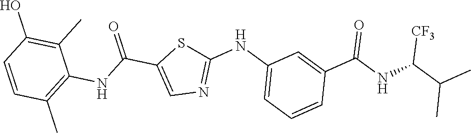 [SMILES: Cc1ccc(O)c(C)c1NC(=O)c1cnc(Nc2cccc(C(=O)N[C@@H](C(C)C)C(F)(F)F)c2)s1] |
| BAA- 260 | 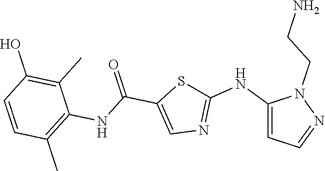 [SMILES: Cc1ccc(O)c(C)c1NC(=O)c1cnc(Nc2ccnn2CCN)s1] |
| BAA- 261 | 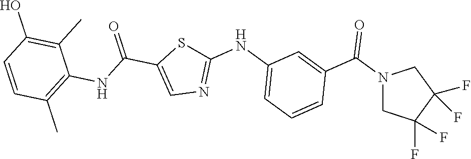 [SMILES: Cc1ccc(O)c(C)c1NC(=O)c1cnc(Nc2cccc(C(=O)N3CC(F)(F)C(F)(F)C3)c2)s1] |
| BAA- 262 | 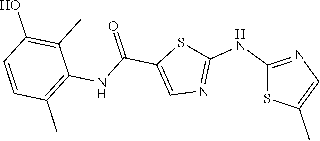 [SMILES: Cc1cnc(Nc2ncc(C(=O)Nc3c(C)ccc(O)c3C)s2)s1] |
| BAA- 263 | 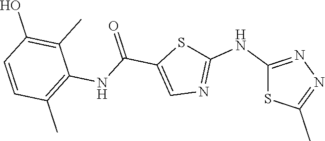 [SMILES: Cc1nnc(Nc2ncc(C(=O)Nc3c(C)ccc(O)c3C)s2)s1] |
| BAA- 264 | 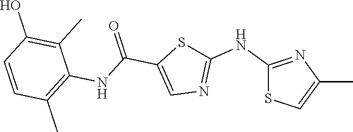 [SMILES: Cc1csc(Nc2ncc(C(=O)Nc3c(C)ccc(O)c3C)s2)n1] |
| BAA- 265 | 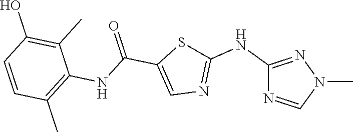 [SMILES: Cc1ccc(O)c(C)c1NC(=O)c1cnc(Nc2ncn(C)n2)s1] |
| BAA- 266 | 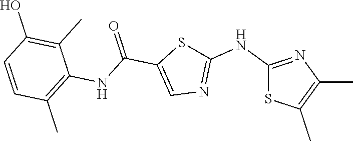 [SMILES: Cc1ccc(O)c(C)c1NC(=O)c1cnc(Nc2nc(C)c(C)s2)s1] |
| BAA- 267 | 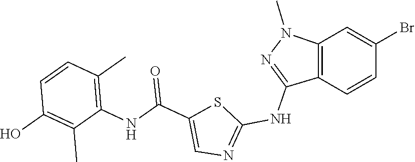 [SMILES: Cc1ccc(O)c(C)c1NC(=O)c1cnc(Nc2nn(C)c3cc(Br)ccc23)s1] |
| BAA- 268 | 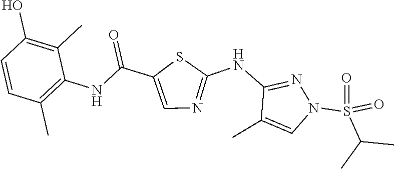 [SMILES: Cc1cn(S(=O)(=O)C(C)C)nc1Nc1ncc(C(=O)Nc2c(C)ccc(O)c2C)s1] |
| BAA- 269 | 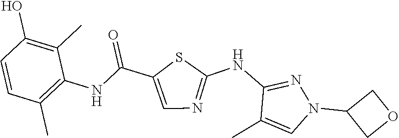 [SMILES: Cc1cn(C2COC2)nc1Nc1ncc(C(=O)Nc2c(C)ccc(O)c2C)s1] |
| BAA- 270 | 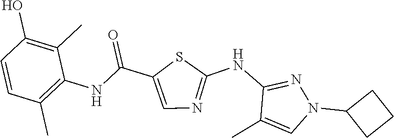 [SMILES: Cc1cn(C2CCC2)nc1Nc1ncc(C(=O)Nc2c(C)ccc(O)c2C)s1] |
| BAA- 271 | 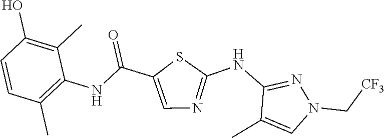 [SMILES: Cc1cn(CC(F)(F)F)nc1Nc1ncc(C(=O)Nc2c(C)ccc(O)c2C)s1] |
| BAA- 272 | 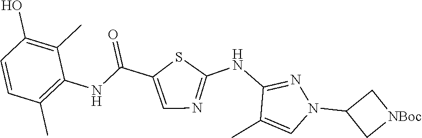 [SMILES: Cc1cn(C2CN(C(=O)OC(C)(C)C)C2)nc1Nc1ncc(C(=O)Nc2c(C)ccc(O)c2C)s1] |
| BAA- 273 | 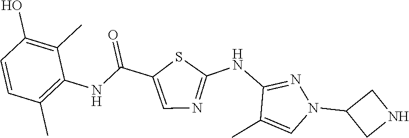 [SMILES: Cc1cn(C2CNC2)nc1Nc1ncc(C(=O)Nc2c(C)ccc(O)c2C)s1] |
| BAA- 274 | 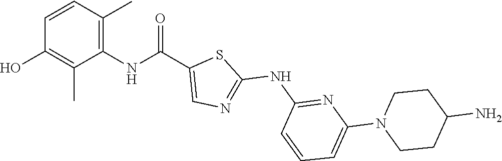 [SMILES: Cc1ccc(O)c(C)c1NC(=O)c1cnc(Nc2cccc(N3CCC(N)CC3)n2)s1] |
| BAA- 275 | 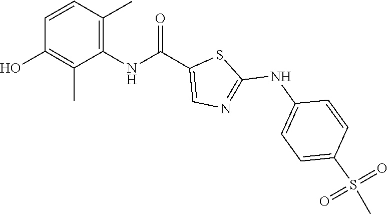 [SMILES: Cc1ccc(O)c(C)c1NC(=O)c1cnc(Nc2ccc(S(C)(=O)=O)cc2)s1] |
| BAA- 276 | 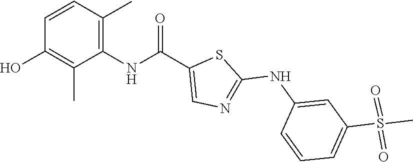 [SMILES: Cc1ccc(O)c(C)c1NC(=O)c1cnc(Nc2cccc(S(C)(=O)=O)c2)s1] |
| BAA- 277 | 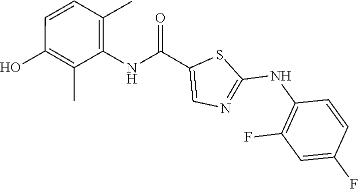 [SMILES: Cc1ccc(O)c(C)c1NC(=O)c1cnc(Nc2ccc(F)cc2F)s1] |
| BAA- 278 | 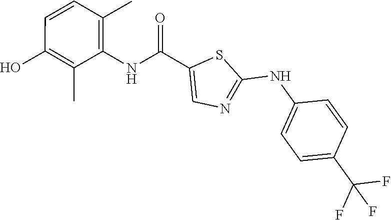 [SMILES: Cc1ccc(O)c(C)c1NC(=O)c1cnc(Nc2ccc(C(F)(F)F)cc2)s1] |
| BAA- 279 | 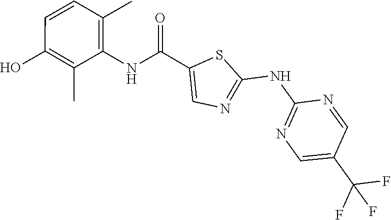 [SMILES: Cc1ccc(O)c(C)c1NC(=O)c1cnc(Nc2ncc(C(F)(F)F)cn2)s1] |
| BAA- 280 | 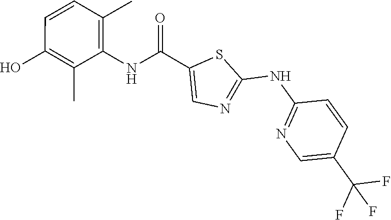 [SMILES: Cc1ccc(O)c(C)c1NC(=O)c1cnc(Nc2ccc(C(F)(F)F)cn2)s1] |
| BAA- 281 | 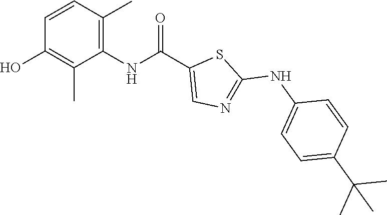 [SMILES: Cc1ccc(O)c(C)c1NC(=O)c1cnc(Nc2ccc(C(C)(C)C)cc2)s1] |
| BAA- 282 | 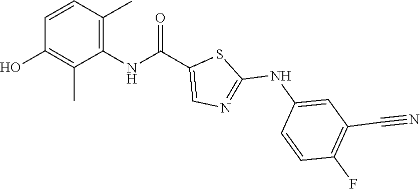 [SMILES: Cc1ccc(O)c(C)c1NC(=O)c1cnc(Nc2ccc(F)c(C#N)c2)s1] |
| BAA- 283 | 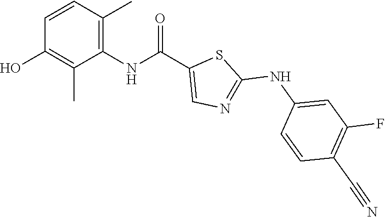 [SMILES: Cc1ccc(O)c(C)c1NC(=O)c1cnc(Nc2ccc(C#N)c(F)c2)s1] |
| BAA- 284 | 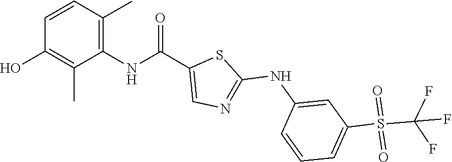 [SMILES: Cc1ccc(O)c(C)c1NC(=O)c1cnc(Nc2cccc(S(=O)(=O)C(F)(F)F)c2)s1] |
| BAA- 285 | 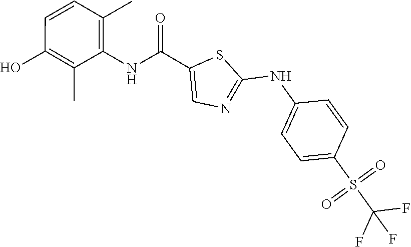 [SMILES: Cc1ccc(O)c(C)c1NC(=O)c1cnc(Nc2ccc(S(=O)(=O)C(F)(F)F)cc2)s1] |
| BAA- 286 | 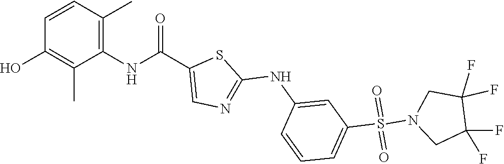 [SMILES: Cc1ccc(O)c(C)c1NC(=O)c1cnc(Nc2cccc(S(=O)(=O)N3CC(F)(F)C(F)(F)C3)c2)s1] |
| BAA- 287 | 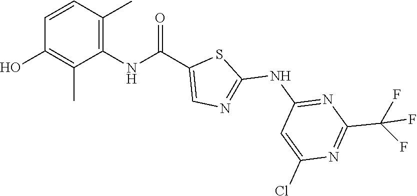 [SMILES: Cc1ccc(O)c(C)c1NC(=O)c1cnc(Nc2cc(Cl)nc(C(F)(F)F)n2)s1] |
| BAA- 288 | 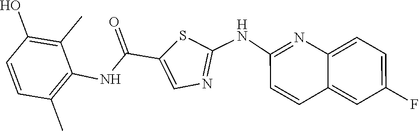 [SMILES: Cc1ccc(O)c(C)c1NC(=O)c1cnc(Nc2ccc3cc(F)ccc3n2)s1] |
| BAA- 289 | 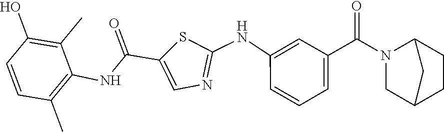 [SMILES: Cc1ccc(O)c(C)c1NC(=O)c1cnc(Nc2cccc(C(=O)N3CC4CCC3C4)c2)s1] |
| BAA- 290 | 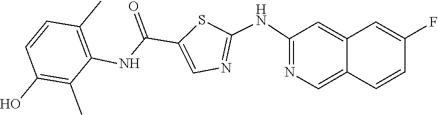 [SMILES: Cc1ccc(O)c(C)c1NC(=O)c1cnc(Nc2cc3cc(F)ccc3cn2)s1] |
| BAA- 291 | 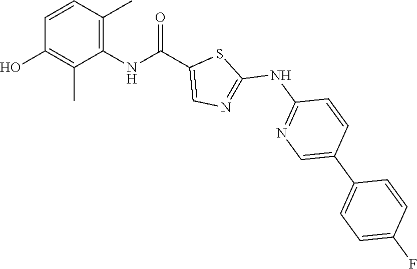 [SMILES: Cc1ccc(O)c(C)c1NC(=O)c1cnc(Nc2ccc(-c3ccc(F)cc3)cn2)s1] |
| BAA- 292 | 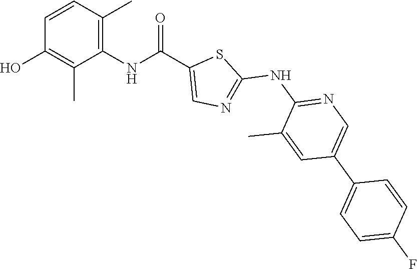 [SMILES: Cc1cc(-c2ccc(F)cc2)cnc1Nc1ncc(C(=O)Nc2c(C)ccc(O)c2C)s1] |
| BAA- 293 | 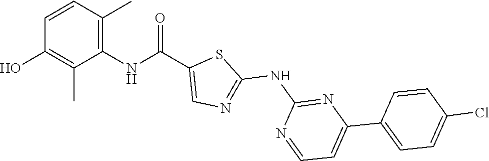 [SMILES: Cc1ccc(O)c(C)c1NC(=O)c1cnc(Nc2nccc(-c3ccc(Cl)cc3)n2)s1] |
| BAA- 294 | 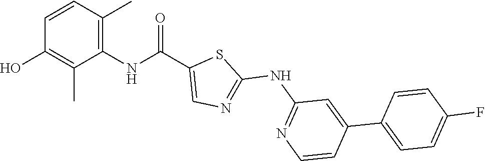 [SMILES: Cc1ccc(O)c(C)c1NC(=O)c1cnc(Nc2cc(-c3ccc(F)cc3)ccn2)s1] |
| BAA- 295 | 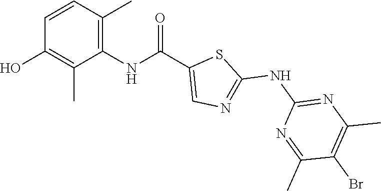 [SMILES: Cc1ccc(O)c(C)c1NC(=O)c1cnc(Nc2nc(C)c(Br)c(C)n2)s1] |
| BAA- 296 | 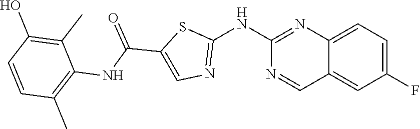 [SMILES: Cc1ccc(O)c(C)c1NC(=O)c1cnc(Nc2ncc3cc(F)ccc3n2)s1] |
| BAA- 297 | 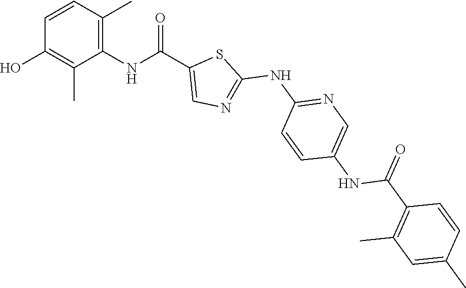 [SMILES: Cc1ccc(C(=O)Nc2ccc(Nc3ncc(C(=O)Nc4c(C)ccc(O)c4C)s3)nc2)c(C)c1] |
| BAA- 298 | 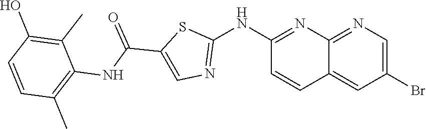 [SMILES: Cc1ccc(O)c(C)c1NC(=O)c1cnc(Nc2ccc3cc(Br)cnc3n2)s1] |
| BAA- 299 | 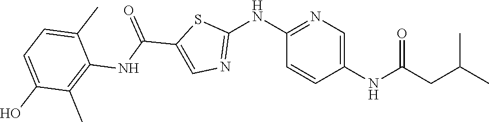 [SMILES: Cc1ccc(O)c(C)c1NC(=O)c1cnc(Nc2ccc(NC(=O)CC(C)C)cn2)s1] |
| BAA- 300 | 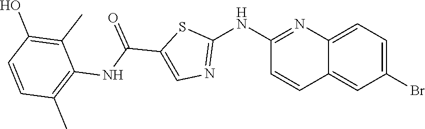 [SMILES: Cc1ccc(O)c(C)c1NC(=O)c1cnc(Nc2ccc3cc(Br)ccc3n2)s1] |
| BAA- 301 | 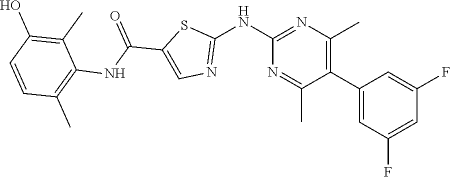 [SMILES: Cc1ccc(O)c(C)c1NC(=O)c1cnc(Nc2nc(C)c(-c3cc(F)cc(F)c3)c(C)n2)s1] |
| BAA- 302 | 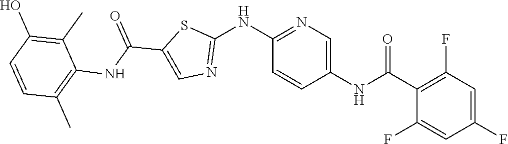 [SMILES: Cc1ccc(O)c(C)c1NC(=O)c1cnc(Nc2ccc(NC(=O)c3c(F)cc(F)cc3F)cn2)s1] |
| BAA- 303 | 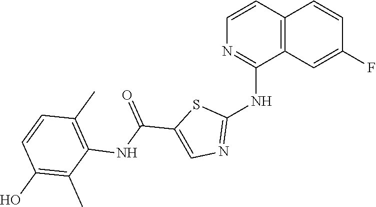 [SMILES: Cc1ccc(O)c(C)c1NC(=O)c1cnc(Nc2nccc3ccc(F)cc23)s1] |
| BAA- 304 | 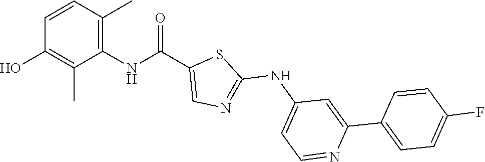 [SMILES: Cc1ccc(O)c(C)c1NC(=O)c1cnc(Nc2ccnc(-c3ccc(F)cc3)c2)s1] |
| BAA- 305 | 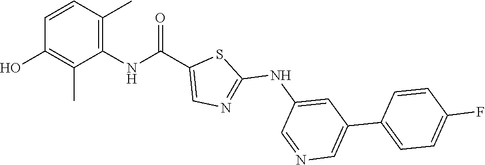 [SMILES: Cc1ccc(O)c(C)c1NC(=O)c1cnc(Nc2cncc(-c3ccc(F)cc3)c2)s1] |
| BAA- 306 | 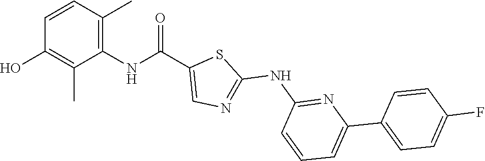 [SMILES: Cc1ccc(O)c(C)c1NC(=O)c1cnc(Nc2cccc(-c3ccc(F)cc3)n2)s1] |
| BAA- 307 | 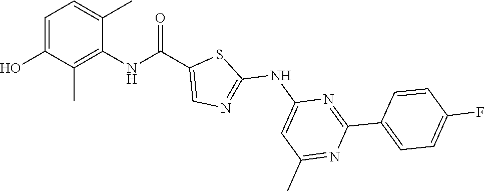 [SMILES: Cc1cc(Nc2ncc(C(=O)Nc3c(C)ccc(O)c3C)s2)nc(-c2ccc(F)cc2)n1] |
| BAA- 308 | 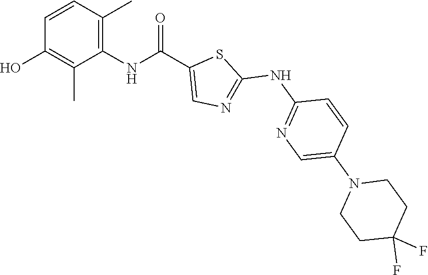 [SMILES: Cc1ccc(O)c(C)c1NC(=O)c1cnc(Nc2ccc(N3CCC(F)(F)CC3)cn2)s1] |
| BAA- 309 | 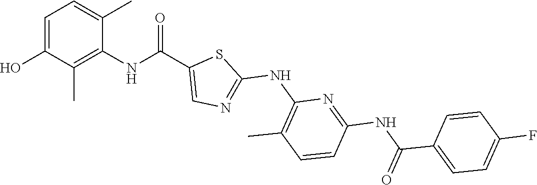 [SMILES: Cc1ccc(NC(=O)c2ccc(F)cc2)nc1Nc1ncc(C(=O)Nc2c(C)ccc(O)c2C)s1] |
| BAA- 310 | 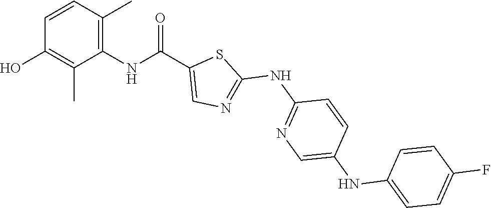 [SMILES: Cc1ccc(O)c(C)c1NC(=O)c1cnc(Nc2ccc(Nc3ccc(F)cc3)cn2)s1] |
| BAA- 311 | 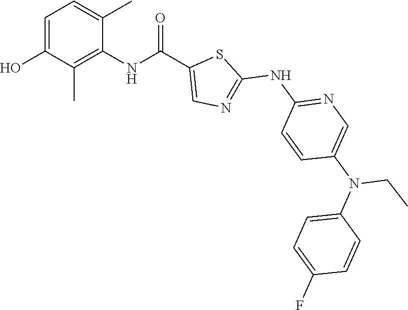 [SMILES: CCN(c1ccc(F)cc1)c1ccc(Nc2ncc(C(=O)Nc3c(C)ccc(O)c3C)s2)nc1] |
| BAA- 312 | 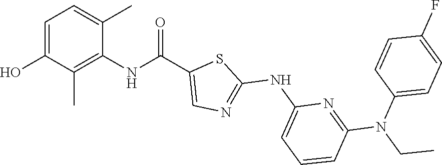 [SMILES: CCN(c1ccc(F)cc1)c1cccc(Nc2ncc(C(=O)Nc3c(C)ccc(O)c3C)s2)n1] |
| BAA- 313 | 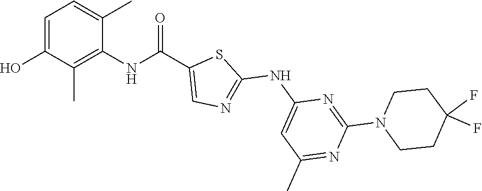 [SMILES: Cc1cc(Nc2ncc(C(=O)Nc3c(C)ccc(O)c3C)s2)nc(N2CCC(F)(F)CC2)n1] |
| BAA- 314 | 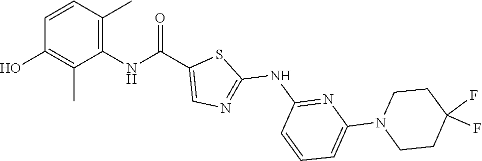 [SMILES: Cc1ccc(O)c(C)c1NC(=O)c1cnc(Nc2cccc(N3CCC(F)(F)CC3)n2)s1] |
| BAA- 315 | 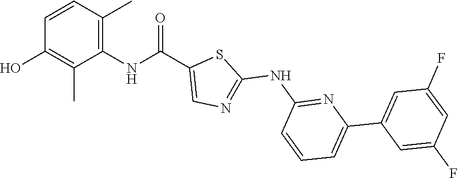 [SMILES: Cc1ccc(O)c(C)c1NC(=O)c1cnc(Nc2cccc(-c3cc(F)cc(F)c3)n2)s1] |
| BAA- 316 | 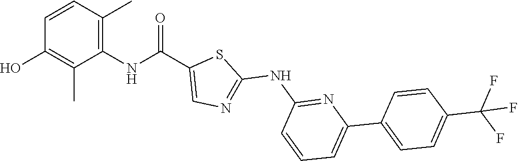 [SMILES: Cc1ccc(O)c(C)c1NC(=O)c1cnc(Nc2cccc(-c3ccc(C(F)(F)F)cc3)n2)s1] |
| BAA- 317 | 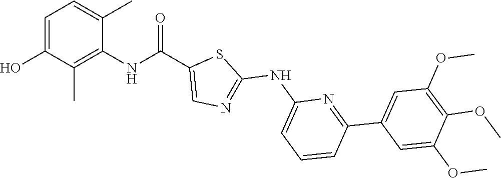 [SMILES: COc1cc(-c2cccc(Nc3ncc(C(=O)Nc4c(C)ccc(O)c4C)s3)n2)cc(OC)c1OC] |
| BAA- 318 | 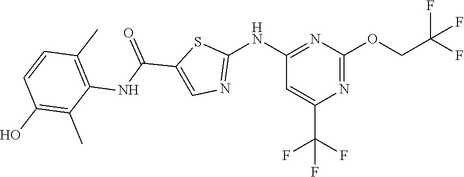 [SMILES: Cc1ccc(O)c(C)c1NC(=O)c1cnc(Nc2cc(C(F)(F)F)nc(OCC(F)(F)F)n2)s1] |
| BAA- 319 | 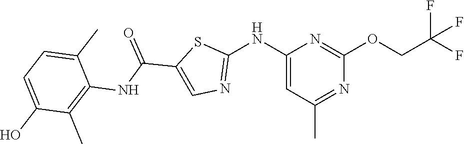 [SMILES: Cc1cc(Nc2ncc(C(=O)Nc3c(C)ccc(O)c3C)s2)nc(OCC(F)(F)F)n1] |
| BAA- 320 | 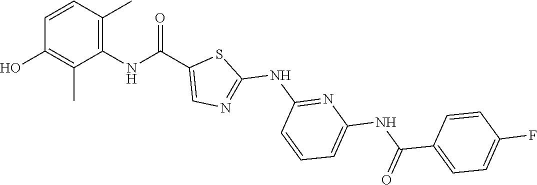 [SMILES: Cc1ccc(O)c(C)c1NC(=O)c1cnc(Nc2cccc(NC(=O)c3ccc(F)cc3)n2)s1] |
| BAA- 321 | 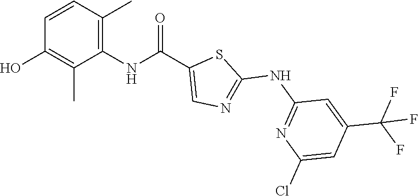 [SMILES: Cc1ccc(O)c(C)c1NC(=O)c1cnc(Nc2cc(C(F)(F)F)cc(Cl)n2)s1] |
| BAA- 322 | 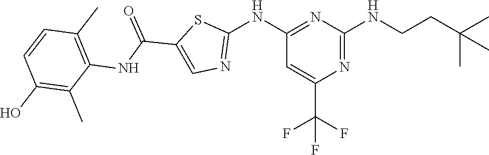 [SMILES: Cc1ccc(O)c(C)c1NC(=O)c1cnc(Nc2cc(C(F)(F)F)nc(NCCC(C)(C)C)n2)s1] |
| BAA- 323 | 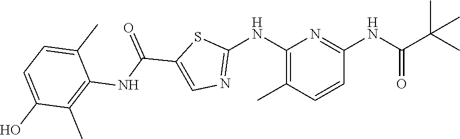 [SMILES: Cc1ccc(NC(=O)C(C)(C)C)nc1Nc1ncc(C(=O)Nc2c(C)ccc(O)c2C)s1] |
| BAA- 324 | 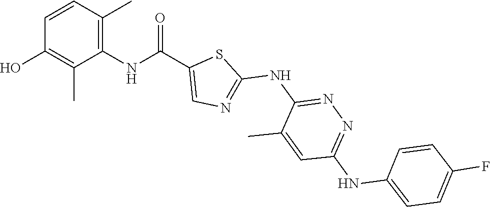 [SMILES: Cc1cc(Nc2ccc(F)cc2)nnc1Nc1ncc(C(=O)Nc2c(C)ccc(O)c2C)s1] |
| BAA- 325 | 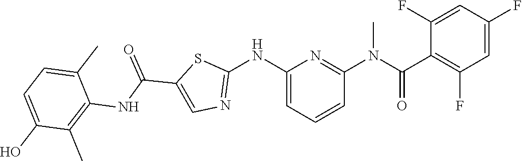 [SMILES: Cc1ccc(O)c(C)c1NC(=O)c1cnc(Nc2cccc(N(C)C(=O)c3c(F)cc(F)cc3F)n2)s1] |
| BAA- 326 | 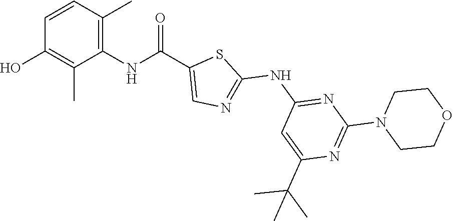 [SMILES: Cc1ccc(O)c(C)c1NC(=O)c1cnc(Nc2cc(C(C)(C)C)nc(N3CCOCC3)n2)s1] |
| BAA- 327 | 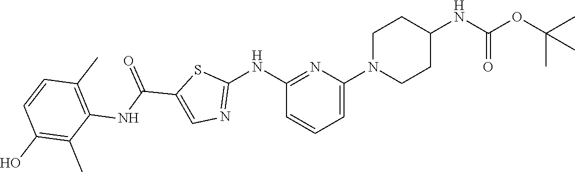 [SMILES: Cc1ccc(O)c(C)c1NC(=O)c1cnc(Nc2cccc(N3CCC(NC(=O)OC(C)(C)C)CC3)n2)s1] |
| BAA- 328 | 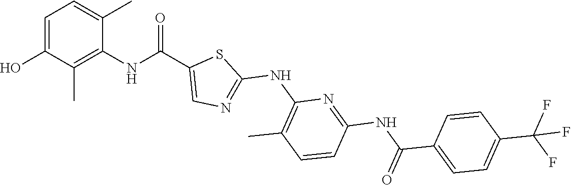 [SMILES: Cc1ccc(NC(=O)c2ccc(C(F)(F)F)cc2)nc1Nc1ncc(C(=O)Nc2c(C)ccc(O)c2C)s1] |
| BAA- 329 | 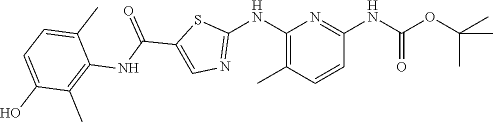 [SMILES: Cc1ccc(NC(=O)OC(C)(C)C)nc1Nc1ncc(C(=O)Nc2c(C)ccc(O)c2C)s1] |
| BAA- 330 | 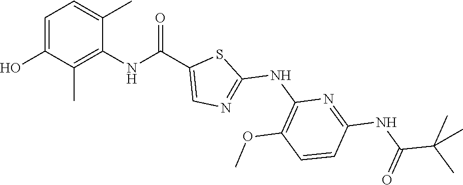 [SMILES: COc1ccc(NC(=O)C(C)(C)C)nc1Nc1ncc(C(=O)Nc2c(C)ccc(O)c2C)s1] |
| BAA- 331 | 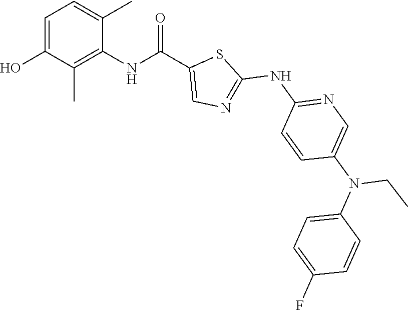 [SMILES: CCN(c1ccc(F)cc1)c1ccc(Nc2ncc(C(=O)Nc3c(C)ccc(O)c3C)s2)nc1] |
| BAA- 332 | 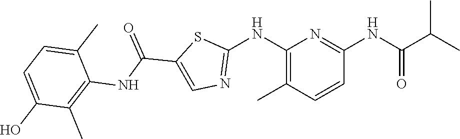 [SMILES: Cc1ccc(NC(=O)C(C)C)nc1Nc1ncc(C(=O)Nc2c(C)ccc(O)c2C)s1] |
| BAA- 333 | 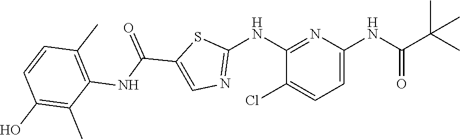 [SMILES: Cc1ccc(O)c(C)c1NC(=O)c1cnc(Nc2nc(NC(=O)C(C)(C)C)ccc2Cl)s1] |
| BAA- 334 | 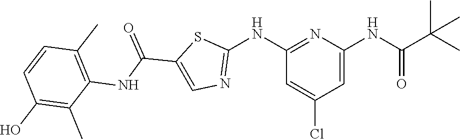 [SMILES: Cc1ccc(O)c(C)c1NC(=O)c1cnc(Nc2cc(Cl)cc(NC(=O)C(C)(C)C)n2)s1] |
| BAA- 335 | 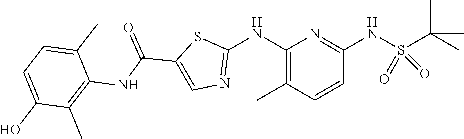 [SMILES: Cc1ccc(NS(=O)(=O)C(C)(C)C)nc1Nc1ncc(C(=O)Nc2c(C)ccc(O)c2C)s1] |
| BAA- 336 | 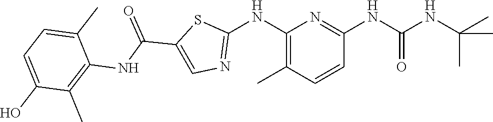 [SMILES: Cc1ccc(NC(=O)NC(C)(C)C)nc1Nc1ncc(C(=O)Nc2c(C)ccc(O)c2C)s1] |
| BAA- 337 | 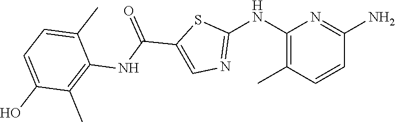 [SMILES: Cc1ccc(N)nc1Nc1ncc(C(=O)Nc2c(C)ccc(O)c2C)s1] |
| BAA- 338 | 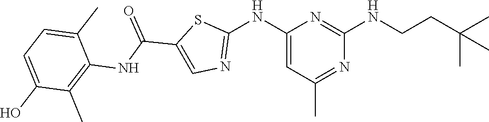 [SMILES: Cc1cc(Nc2ncc(C(=O)Nc3c(C)ccc(O)c3C)s2)nc(NCCC(C)(C)C)n1] |
| BAA- 339 | 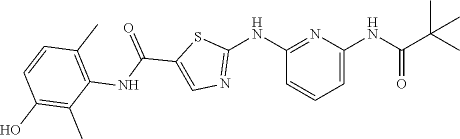 [SMILES: Cc1ccc(O)c(C)c1NC(=O)c1cnc(Nc2cccc(NC(=O)C(C)(C)C)n2)s1] |
| BAA- 340 | 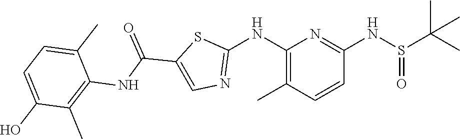 [SMILES: Cc1ccc(NS(=O)C(C)(C)C)nc1Nc1ncc(C(=O)Nc2c(C)ccc(O)c2C)s1] |
| BAA- 341 | 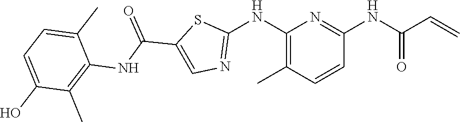 [SMILES: C=CC(=O)Nc1ccc(C)c(Nc2ncc(C(=O)Nc3c(C)ccc(O)c3C)s2)n1] |
| BAA- 342 | 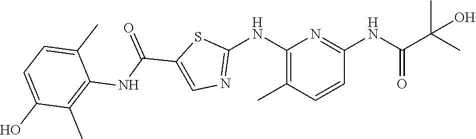 [SMILES: Cc1ccc(NC(=O)C(C)(C)O)nc1Nc1ncc(C(=O)Nc2c(C)ccc(O)c2C)s1] |
| BAA- 343 | 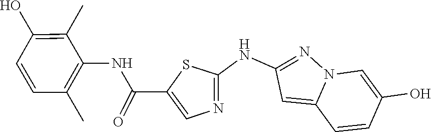 [SMILES: Cc1ccc(O)c(C)c1NC(=O)c1cnc(Nc2cc3ccc(O)cn3n2)s1] |
| BAA- 344 | 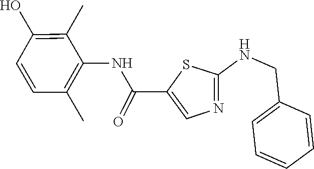 [SMILES: Cc1ccc(O)c(C)c1NC(=O)c1cnc(NCc2ccccc2)s1] |
| BAA- 345 | 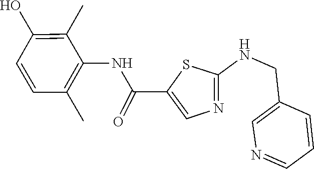 [SMILES: Cc1ccc(O)c(C)c1NC(=O)c1cnc(NCc2cccnc2)s1] |
| BAA- 346 | 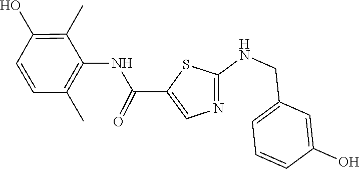 [SMILES: Cc1ccc(O)c(C)c1NC(=O)c1cnc(NCc2cccc(O)c2)s1] |
| BAA- 347 | 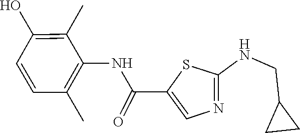 [SMILES: Cc1ccc(O)c(C)c1NC(=O)c1cnc(NCC2CC2)s1] |
| BAA- 348 | 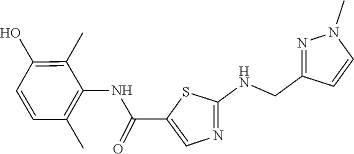 [SMILES: Cc1ccc(O)c(C)c1NC(=O)c1cnc(NCc2ccn(C)n2)s1] |
| BAA- 349 | 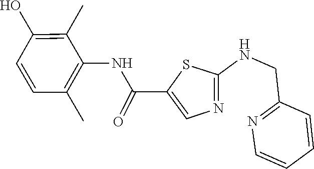 [SMILES: Cc1ccc(O)c(C)c1NC(=O)c1cnc(NCc2ccccn2)s1] |
| BAA- 350 | 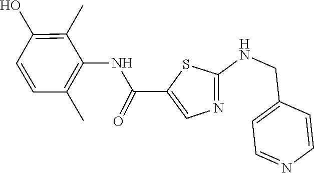 [SMILES: Cc1ccc(O)c(C)c1NC(=O)c1cnc(NCc2ccncc2)s1] |
| BAA- 351 | 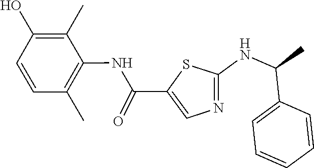 [SMILES: Cc1ccc(O)c(C)c1NC(=O)c1cnc(N[C@@H](C)c2ccccc2)s1] |
| BAA- 352 | 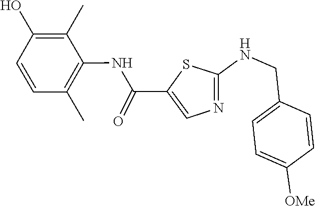 [SMILES: COc1ccc(CNc2ncc(C(=O)Nc3c(C)ccc(O)c3C)s2)cc1] |
| BAA- 353 | 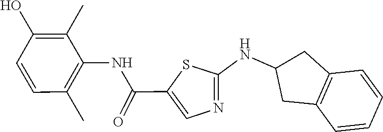 [SMILES: Cc1ccc(O)c(C)c1NC(=O)c1cnc(NC2Cc3ccccc3C2)s1] |
| BAA- 354 | 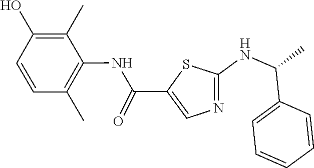 [SMILES: Cc1ccc(O)c(C)c1NC(=O)c1cnc(N[C@H](C)c2ccccc2)s1] |
| BAA- 355 | 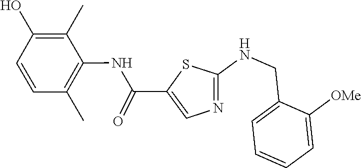 [SMILES: COc1ccccc1CNc1ncc(C(=O)Nc2c(C)ccc(O)c2C)s1] |
| BAA- 356 | 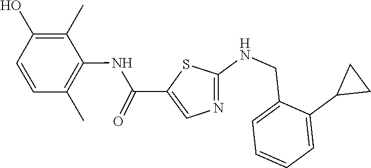 [SMILES: Cc1ccc(O)c(C)c1NC(=O)c1cnc(NCc2ccccc2C2CC2)s1] |
| BAA- 357 | 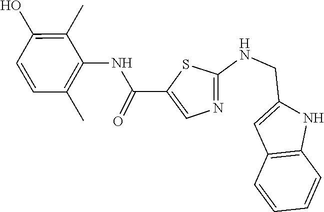 [SMILES: Cc1ccc(O)c(C)c1NC(=O)c1cnc(NCc2cc3ccccc3[nH]2)s1] |
| BAA- 358 | 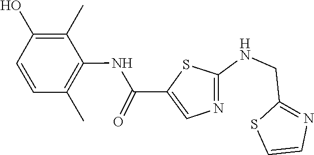 [SMILES: Cc1ccc(O)c(C)c1NC(=O)c1cnc(NCc2nccs2)s1] |
| BAA- 359 | 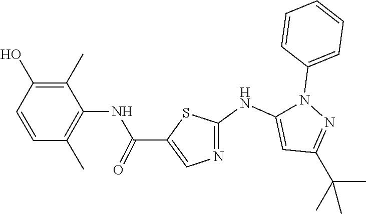 [SMILES: Cc1ccc(O)c(C)c1NC(=O)c1cnc(Nc2cc(C(C)(C)C)nn2-c2ccccc2)s1] |
| BAA- 360 | 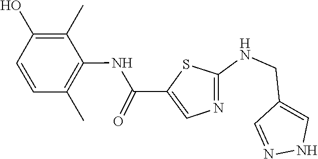 [SMILES: Cc1ccc(O)c(C)c1NC(=O)c1cnc(NCc2cn[nH]c2)s1] |
| BAA- 361 | 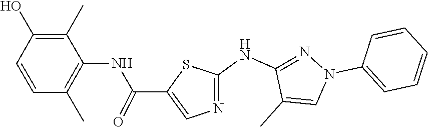 [SMILES: Cc1cn(-c2ccccc2)nc1Nc1ncc(C(=O)Nc2c(C)ccc(O)c2C)s1] |
| BAA- 362 | 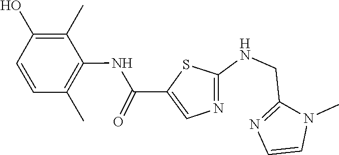 [SMILES: Cc1ccc(O)c(C)c1NC(=O)c1cnc(NCc2nccn2C)s1] |
| BAA- 363 | 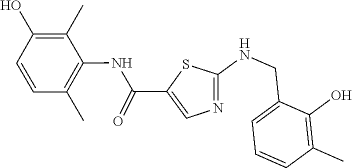 [SMILES: Cc1cccc(CNc2ncc(C(=O)Nc3c(C)ccc(O)c3C)s2)c1O] |
| BAA- 364 | 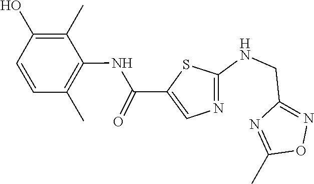 [SMILES: Cc1nc(CNc2ncc(C(=O)Nc3c(C)ccc(O)c3C)s2)no1] |
| BAA- 365 | 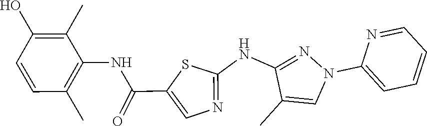 [SMILES: Cc1cn(-c2ccccn2)nc1Nc1ncc(C(=O)Nc2c(C)ccc(O)c2C)s1] |
| BAA- 366 | 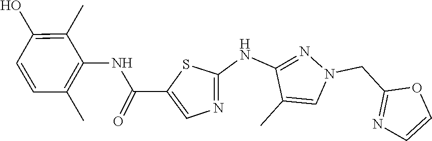 [SMILES: Cc1cn(Cc2ncco2)nc1Nc1ncc(C(=O)Nc2c(C)ccc(O)c2C)s1] |
| BAA- 367 | 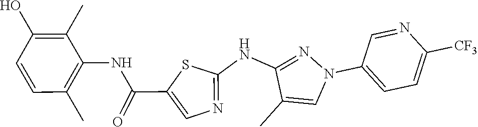 [SMILES: Cc1cn(-c2ccc(C(F)(F)F)nc2)nc1Nc1ncc(C(=O)Nc2c(C)ccc(O)c2C)s1] |
| BAA- 368 | 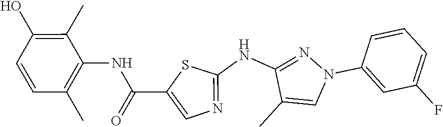 [SMILES: Cc1cn(-c2cccc(F)c2)nc1Nc1ncc(C(=O)Nc2c(C)ccc(O)c2C)s1] |
| BAA- 369 | 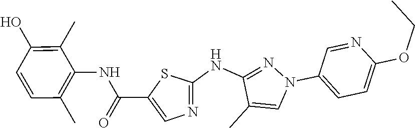 [SMILES: CCOc1ccc(-n2cc(C)c(Nc3ncc(C(=O)Nc4c(C)ccc(O)c4C)s3)n2)cn1] |
| BAA- 370 | 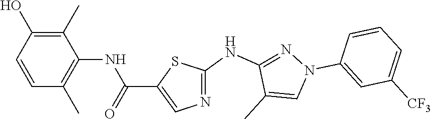 [SMILES: Cc1cn(-c2cccc(C(F)(F)F)c2)nc1Nc1ncc(C(=O)Nc2c(C)ccc(O)c2C)s1] |
| BAA- 371 | 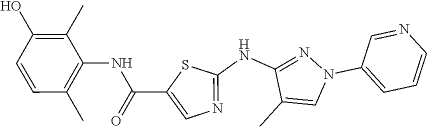 [SMILES: Cc1cn(-c2cccnc2)nc1Nc1ncc(C(=O)Nc2c(C)ccc(O)c2C)s1] |
| BAA- 372 | 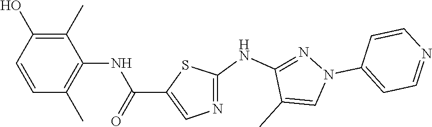 [SMILES: Cc1cn(-c2ccncc2)nc1Nc1ncc(C(=O)Nc2c(C)ccc(O)c2C)s1] |
| BAA- 373 | 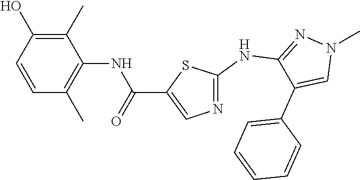 [SMILES: Cc1ccc(O)c(C)c1NC(=O)c1cnc(Nc2nn(C)cc2-c2ccccc2)s1] |
| BAA- 374 | 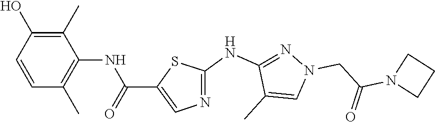 [SMILES: Cc1cn(CC(=O)N2CCC2)nc1Nc1ncc(C(=O)Nc2c(C)ccc(O)c2C)s1] |
| BAA- 375 | 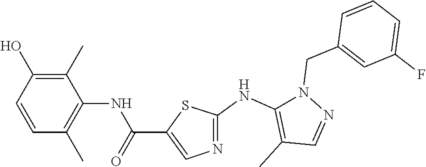 [SMILES: Cc1ccc(O)c(C)c1NC(=O)c1cnc(Nc2c(C)cnn2Cc2cccc(F)c2)s1] |
| BAA- 376 | 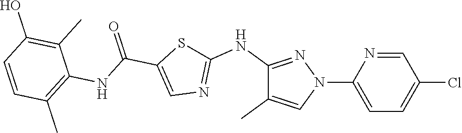 [SMILES: Cc1cn(-c2ccc(Cl)cn2)nc1Nc1ncc(C(=O)Nc2c(C)ccc(O)c2C)s1] |
| BAA- 377 | 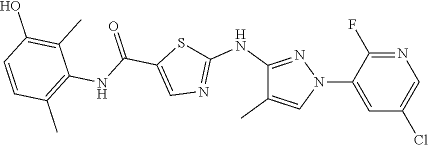 [SMILES: Cc1cn(-c2cc(Cl)cnc2F)nc1Nc1ncc(C(=O)Nc2c(C)ccc(O)c2C)s1] |
| BAA- 378 | 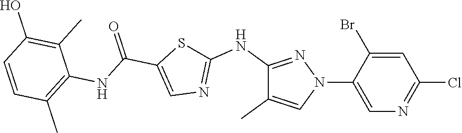 [SMILES: Cc1cn(-c2cnc(Cl)cc2Br)nc1Nc1ncc(C(=O)Nc2c(C)ccc(O)c2C)s1] |
| BAA- 379 | 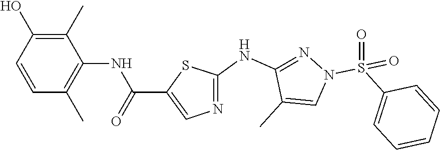 [SMILES: Cc1cn(S(=O)(=O)c2ccccc2)nc1Nc1ncc(C(=O)Nc2c(C)ccc(O)c2C)s1] |
| BAA- 380 | 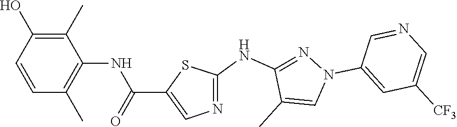 [SMILES: Cc1cn(-c2cncc(C(F)(F)F)c2)nc1Nc1ncc(C(=O)Nc2c(C)ccc(O)c2C)s1] |
| BAA- 381 | 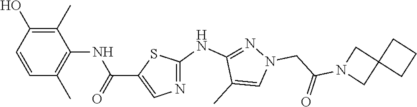 [SMILES: Cc1cn(CC(=O)N2CC3(CCC3)C2)nc1Nc1ncc(C(=O)Nc2c(C)ccc(O)c2C)s1] |
| BAA- 382 | 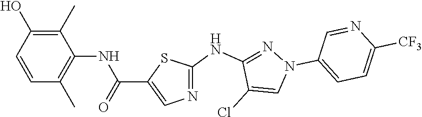 [SMILES: Cc1ccc(O)c(C)c1NC(=O)c1cnc(Nc2nn(-c3ccc(C(F)(F)F)nc3)cc2Cl)s1] |
| BAA- 383 | 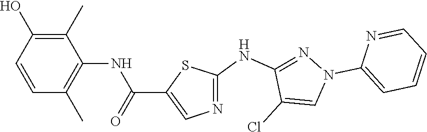 [SMILES: Cc1ccc(O)c(C)c1NC(=O)c1cnc(Nc2nn(-c3ccccn3)cc2Cl)s1] |
| BAA- 384 | 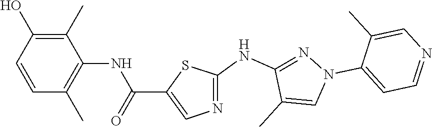 [SMILES: Cc1cnccc1-n1cc(C)c(Nc2ncc(C(=O)Nc3c(C)ccc(O)c3C)s2)n1] |
| BAA- 385 | 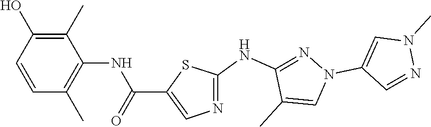 [SMILES: Cc1cn(-c2cnn(C)c2)nc1Nc1ncc(C(=O)Nc2c(C)ccc(O)c2C)s1] |
| BAA- 386 | 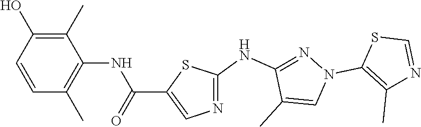 [SMILES: Cc1cn(-c2scnc2C)nc1Nc1ncc(C(=O)Nc2c(C)ccc(O)c2C)s1] |
| BAA- 387 | 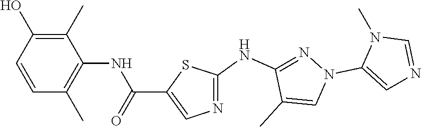 [SMILES: Cc1cn(-c2cncn2C)nc1Nc1ncc(C(=O)Nc2c(C)ccc(O)c2C)s1] |
| BAA- 388 | 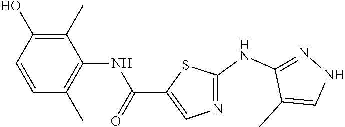 [SMILES: Cc1c[nH]nc1Nc1ncc(C(=O)Nc2c(C)ccc(O)c2C)s1] |
| BAA- 389 | 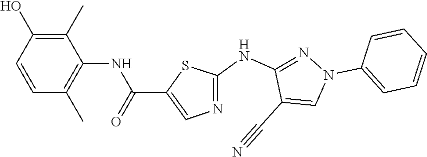 [SMILES: Cc1ccc(O)c(C)c1NC(=O)c1cnc(Nc2nn(-c3ccccc3)cc2C#N)s1] |
| BAA- 390 | 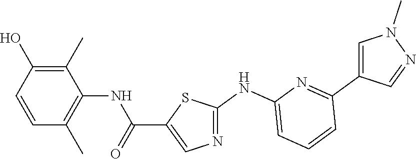 [SMILES: Cc1ccc(O)c(C)c1NC(=O)c1cnc(Nc2cccc(-c3cnn(C)c3)n2)s1] |
| BAA- 391 | 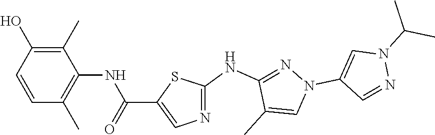 [SMILES: Cc1cn(-c2cnn(C(C)C)c2)nc1Nc1ncc(C(=O)Nc2c(C)ccc(O)c2C)s1] |
| BAA- 392 | 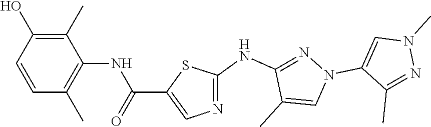 [SMILES: Cc1cn(-c2cn(C)nc2C)nc1Nc1ncc(C(=O)Nc2c(C)ccc(O)c2C)s1] |
| BAA- 393 | 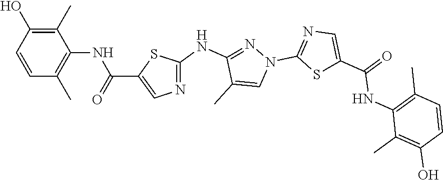 [SMILES: Cc1cn(-c2ncc(C(=O)Nc3c(C)ccc(O)c3C)s2)nc1Nc1ncc(C(=O)Nc2c(C)ccc(O)c2C)s1] |
| BAA- 394 | 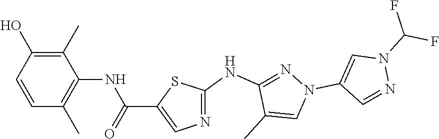 [SMILES: Cc1cn(-c2cnn(C(F)F)c2)nc1Nc1ncc(C(=O)Nc2c(C)ccc(O)c2C)s1] |
| BAA- 395 | 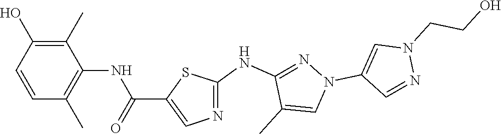 [SMILES: Cc1cn(-c2cnn(CCO)c2)nc1Nc1ncc(C(=O)Nc2c(C)ccc(O)c2C)s1] |
| BAA- 396 | 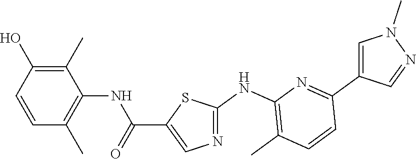 [SMILES: Cc1ccc(-c2cnn(C)c2)nc1Nc1ncc(C(=O)Nc2c(C)ccc(O)c2C)s1] |
| BAA- 397 | 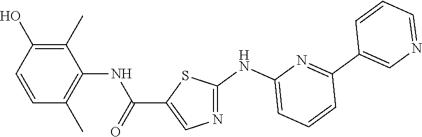 [SMILES: Cc1ccc(O)c(C)c1NC(=O)c1cnc(Nc2cccc(-c3cccnc3)n2)s1] |
| BAA- 398 | 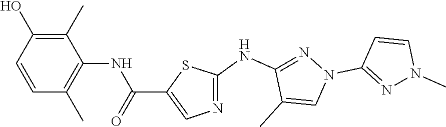 [SMILES: Cc1cn(-c2ccn(C)n2)nc1Nc1ncc(C(=O)Nc2c(C)ccc(O)c2C)s1] |
| BAA- 399 | 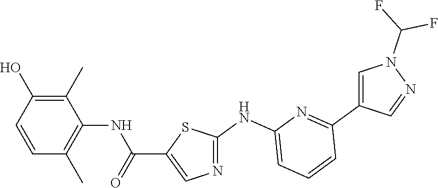 [SMILES: Cc1ccc(O)c(C)c1NC(=O)c1cnc(Nc2cccc(-c3cnn(C(F)F)c3)n2)s1] |
| BAA- 400 | 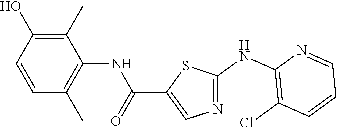 [SMILES: Cc1ccc(O)c(C)c1NC(=O)c1cnc(Nc2ncccc2Cl)s1] |
| BAA- 401 | 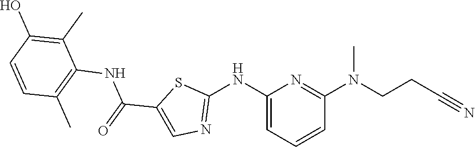 [SMILES: Cc1ccc(O)c(C)c1NC(=O)c1cnc(Nc2cccc(N(C)CCC#N)n2)s1] |
| BAA 402 | 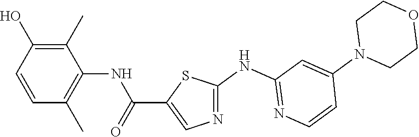 [SMILES: Cc1ccc(O)c(C)c1NC(=O)c1cnc(Nc2cc(N3CCOCC3)ccn2)s1] |
| BAA- 403 | 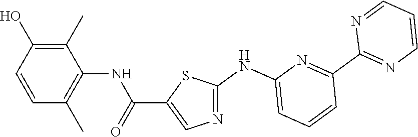 [SMILES: Cc1ccc(O)c(C)c1NC(=O)c1cnc(Nc2cccc(-c3ncccn3)n2)s1] |
| BAA- 404 | 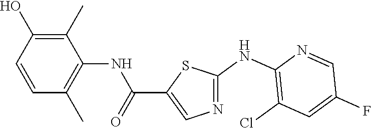 [SMILES: Cc1ccc(O)c(C)c1NC(=O)c1cnc(Nc2ncc(F)cc2Cl)s1] |
| BAA- 405 | 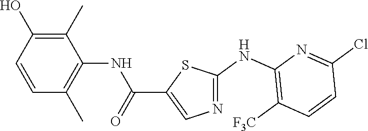 [SMILES: Cc1ccc(O)c(C)c1NC(=O)c1cnc(Nc2nc(Cl)ccc2C(F)(F)F)s1] |
| BAA- 406 | 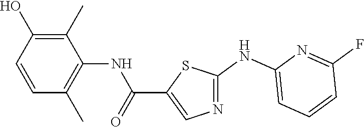 [SMILES: Cc1ccc(O)c(C)c1NC(=O)c1cnc(Nc2cccc(F)n2)s1] |
| BAA- 407 | 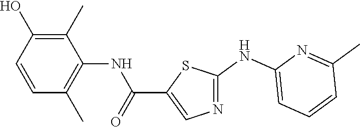 [SMILES: Cc1cccc(Nc2ncc(C(=O)Nc3c(C)ccc(O)c3C)s2)n1] |
| BAA- 408 | 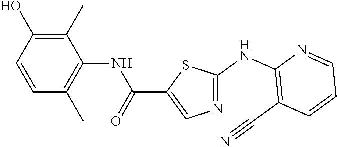 [SMILES: Cc1ccc(O)c(C)c1NC(=O)c1cnc(Nc2ncccc2C#N)s1] |
| BAA- 409 | 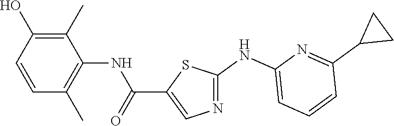 [SMILES: Cc1ccc(O)c(C)c1NC(=O)c1cnc(Nc2cccc(C3CC3)n2)s1] |
| BAA- 410 | 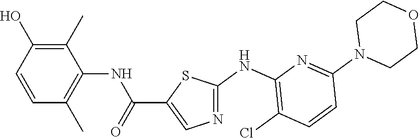 [SMILES: Cc1ccc(O)c(C)c1NC(=O)c1cnc(Nc2nc(N3CCOCC3)ccc2Cl)s1] |
| BAA- 411 | 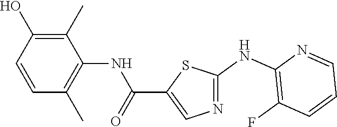 [SMILES: Cc1ccc(O)c(C)c1NC(=O)c1cnc(Nc2ncccc2F)s1] |
| BAA- 412 | 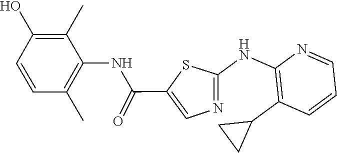 [SMILES: Cc1ccc(O)c(C)c1NC(=O)c1cnc(Nc2ncccc2C2CC2)s1] |
| BAA- 413 | 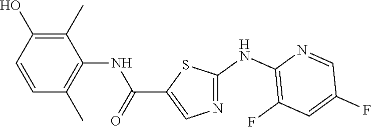 [SMILES: Cc1ccc(O)c(C)c1NC(=O)c1cnc(Nc2ncc(F)cc2F)s1] |
| BAA- 414 | 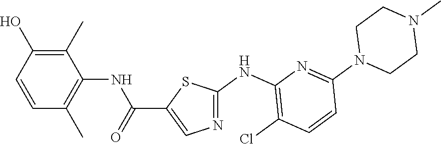 [SMILES: Cc1ccc(O)c(C)c1NC(=O)c1cnc(Nc2nc(N3CCN(C)CC3)ccc2Cl)s1] |
| BAA- 415 | 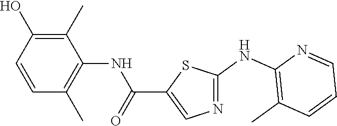 [SMILES: Cc1cccnc1Nc1ncc(C(=O)Nc2c(C)ccc(O)c2C)s1] |
| BAA- 416 | 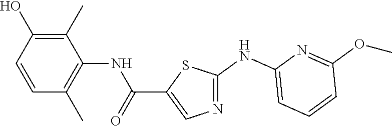 [SMILES: COc1cccc(Nc2ncc(C(=O)Nc3c(C)ccc(O)c3C)s2)n1] |
| BAA- 417 | 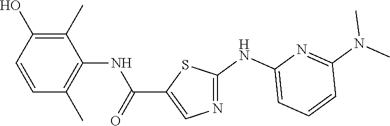 [SMILES: Cc1ccc(O)c(C)c1NC(=O)c1cnc(Nc2cccc(N(C)C)n2)s1] |
| BAA- 418 | 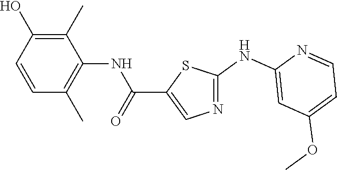 [SMILES: COc1ccnc(Nc2ncc(C(=O)Nc3c(C)ccc(O)c3C)s2)c1] |
| BAA- 419 | 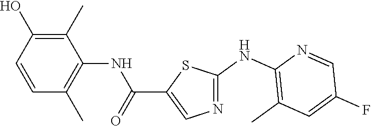 [SMILES: Cc1cc(F)cnc1Nc1ncc(C(=O)Nc2c(C)ccc(O)c2C)s1] |
| BAA- 420 | 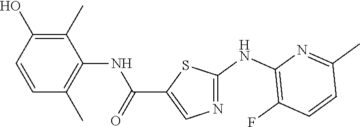 [SMILES: Cc1ccc(F)c(Nc2ncc(C(=O)Nc3c(C)ccc(O)c3C)s2)n1] |
| BAA- 421 | 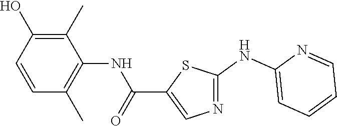 [SMILES: Cc1ccc(O)c(C)c1NC(=O)c1cnc(Nc2ccccn2)s1] |
| BAA- 422 | 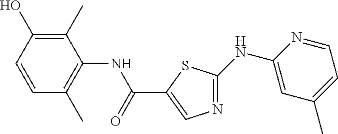 [SMILES: Cc1ccnc(Nc2ncc(C(=O)Nc3c(C)ccc(O)c3C)s2)c1] |
| BAA- 423 | 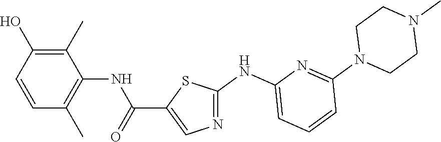 [SMILES: Cc1ccc(O)c(C)c1NC(=O)c1cnc(Nc2cccc(N3CCN(C)CC3)n2)s1] |
| BAA- 424 | 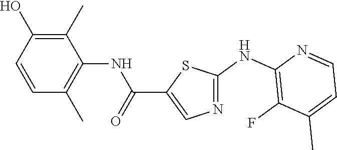 [SMILES: Cc1ccnc(Nc2ncc(C(=O)Nc3c(C)ccc(O)c3C)s2)c1F] |
| BAA- 425 | 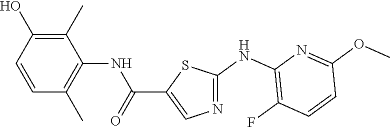 [SMILES: COc1ccc(F)c(Nc2ncc(C(=O)Nc3c(C)ccc(O)c3C)s2)n1] |
| BAA- 426 | 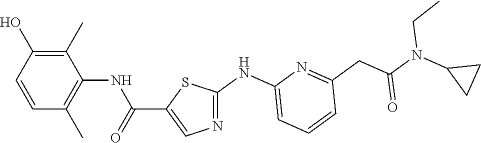 [SMILES: CCN(C(=O)Cc1cccc(Nc2ncc(C(=O)Nc3c(C)ccc(O)c3C)s2)n1)C1CC1] |
| BAA- 427 | 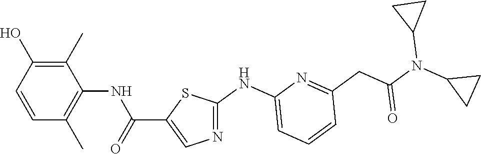 [SMILES: Cc1ccc(O)c(C)c1NC(=O)c1cnc(Nc2cccc(CC(=O)N(C3CC3)C3CC3)n2)s1] |
| BAA- 428 | 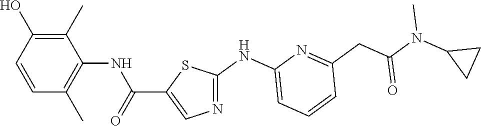 [SMILES: Cc1ccc(O)c(C)c1NC(=O)c1cnc(Nc2cccc(CC(=O)N(C)C3CC3)n2)s1] |
| BAA- 429 | 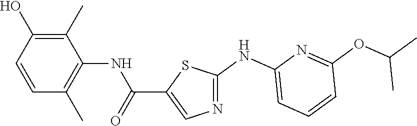 [SMILES: Cc1ccc(O)c(C)c1NC(=O)c1cnc(Nc2cccc(OC(C)C)n2)s1] |
| BAA- 430 | 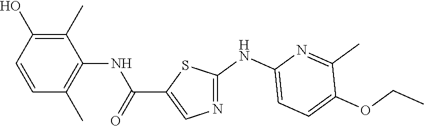 [SMILES: CCOc1ccc(Nc2ncc(C(=O)Nc3c(C)ccc(O)c3C)s2)nc1C] |
| BAA- 431 | 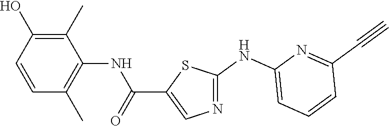 [SMILES: C#Cc1cccc(Nc2ncc(C(=O)Nc3c(C)ccc(O)c3C)s2)n1] |
| BAA- 432 | 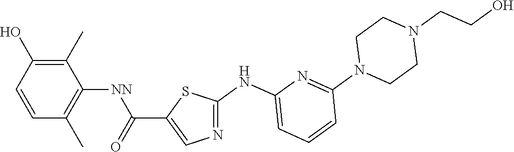 |
| BAA- 433 | 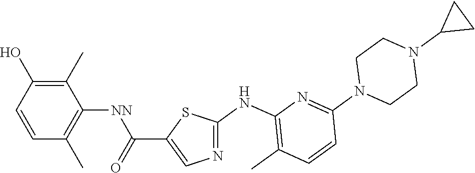 |
| BAA- 434 | 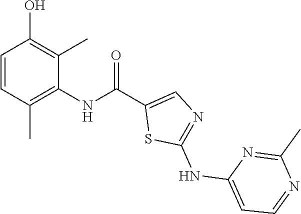 [SMILES: Cc1nccc(Nc2ncc(C(=O)Nc3c(C)ccc(O)c3C)s2)n1] |
| BAA- 435 | 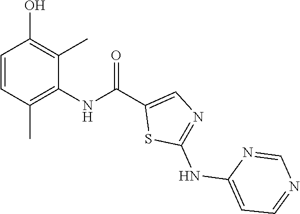 [SMILES: Cc1ccc(O)c(C)c1NC(=O)c1cnc(Nc2ccncn2)s1] |
| BAA- 436 | 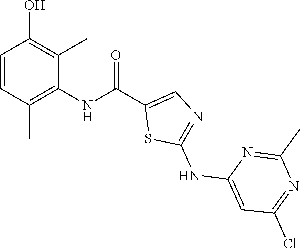 [SMILES: Cc1nc(Cl)cc(Nc2ncc(C(=O)Nc3c(C)ccc(O)c3C)s2)n1] |
| BAA- 437 | 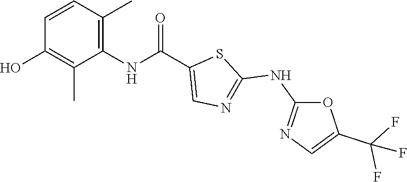 [SMILES: Cc1ccc(O)c(C)c1NC(=O)c1cnc(Nc2ncc(C(F)(F)F)o2)s1] |
| BAA- 438 | 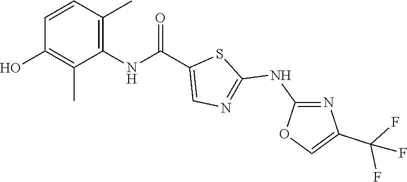 [SMILES: Cc1ccc(O)c(C)c1NC(=O)c1cnc(Nc2nc(C(F)(F)F)co2)s1] |
| BAA- 439 | 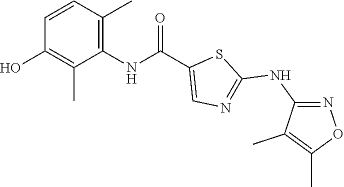 [SMILES: Cc1ccc(O)c(C)c1NC(=O)c1cnc(Nc2noc(C)c2C)s1] |
| BAA- 440 | 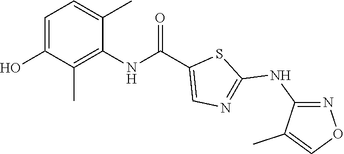 [SMILES: Cc1conc1Nc1ncc(C(=O)Nc2c(C)ccc(O)c2C)s1] |
| BAA- 441 | 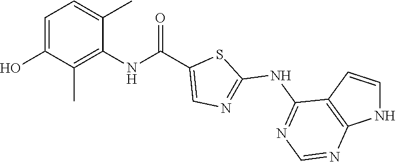 [SMILES: Cc1ccc(O)c(C)c1NC(=O)c1cnc(Nc2ncnc3[nH]ccc23)s1] |
| BAA- 442 | 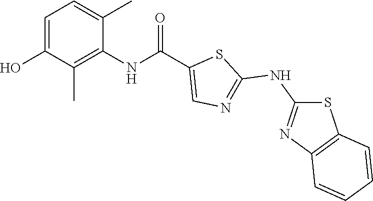 [SMILES: Cc1ccc(O)c(C)c1NC(=O)c1cnc(Nc2nc3ccccc3s2)s1] |
| BAA- 443 | 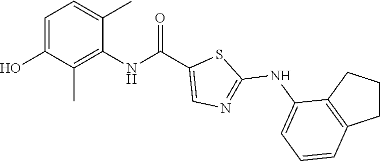 [SMILES: Cc1ccc(O)c(C)c1NC(=O)c1cnc(Nc2cccc3c2CCC3)s1] |
| BAA- 444 | 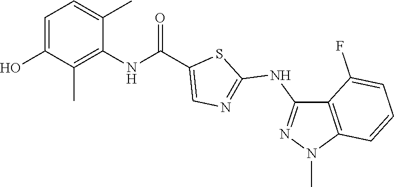 [SMILES: Cc1ccc(O)c(C)c1NC(=O)c1cnc(Nc2nn(C)c3cccc(F)c23)s1] |
| BAA- 445 | 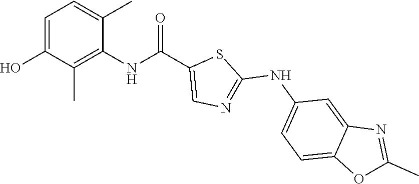 [SMILES: Cc1nc2cc(Nc3ncc(C(=O)Nc4c(C)ccc(O)c4C)s3)ccc2o1] |
| BAA- 446 | 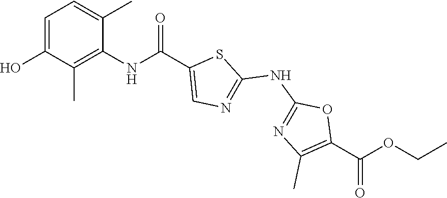 [SMILES: CCOC(=O)c1oc(Nc2ncc(C(=O)Nc3c(C)ccc(O)c3C)s2)nc1C] |
| BAA- 447 | 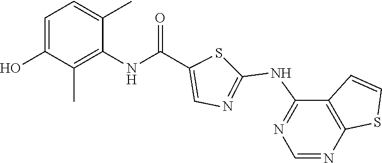 [SMILES: Cc1ccc(O)c(C)c1NC(=O)c1cnc(Nc2ncnc3sccc23)s1] |
| BAA- 448 | 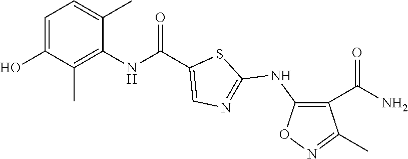 [SMILES: Cc1ccc(O)c(C)c1NC(=O)c1cnc(Nc2onc(C)c2C(N)=O)s1] |
| BAA- 449 | 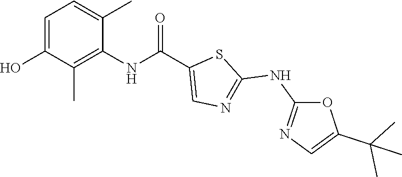 [SMILES: Cc1ccc(O)c(C)c1NC(=O)c1cnc(Nc2ncc(C(C)(C)C)o2)s1] |
| BAA- 450 | 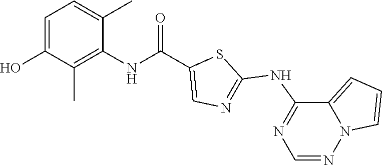 [SMILES: Cc1ccc(O)c(C)c1NC(=O)c1cnc(Nc2ncnn3cccc23)s1] |
| BAA- 451 | 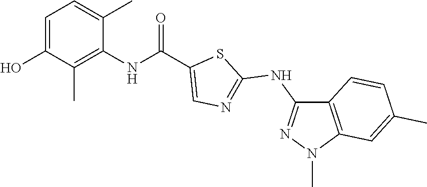 [SMILES: Cc1ccc2c(Nc3ncc(C(=O)Nc4c(C)ccc(O)c4C)s3)nn(C)c2c1] |
| BAA- 452 | 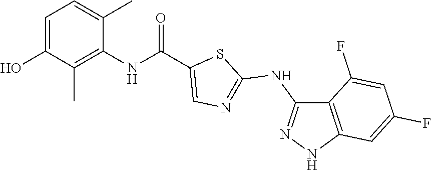 [SMILES: Cc1ccc(O)c(C)c1NC(=O)c1cnc(Nc2n[nH]c3cc(F)cc(F)c23)s1] |
| BAA- 453 | 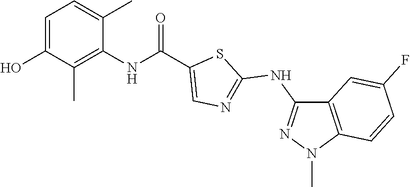 [SMILES: Cc1ccc(O)c(C)c1NC(=O)c1cnc(Nc2nn(C)c3ccc(F)cc23)s1] |
| BAA- 454 | 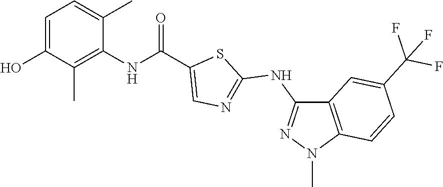 [SMILES: Cc1ccc(O)c(C)c1NC(=O)c1cnc(Nc2nn(C)c3ccc(C(F)(F)F)cc23)s1] |
| BAA- 455 | 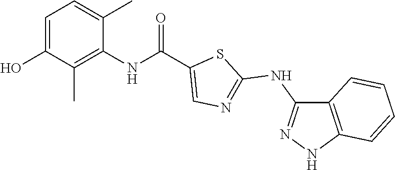 [SMILES: Cc1ccc(O)c(C)c1NC(=O)c1cnc(Nc2n[nH]c3ccccc23)s1] |
| BAA- 456 | 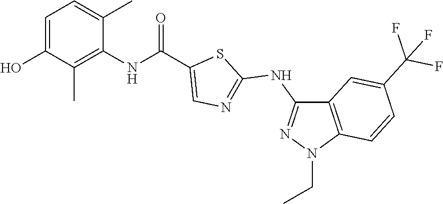 [SMILES: CCn1nc(Nc2ncc(C(=O)Nc3c(C)ccc(O)c3C)s2)c2cc(C(F)(F)F)ccc21] |
| BAA- 457 | 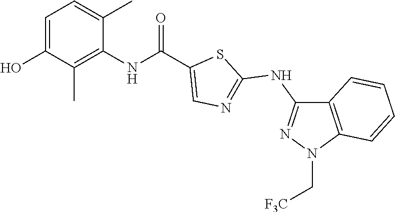 [SMILES: Cc1ccc(O)c(C)c1NC(=O)c1cnc(Nc2nn(CC(F)(F)F)c3ccccc23)s1] |
| BAA- 458 | 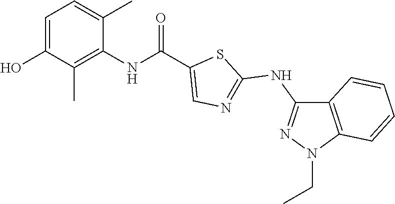 [SMILES: CCn1nc(Nc2ncc(C(=O)Nc3c(C)ccc(O)c3C)s2)c2ccccc21] |
| BAA- 459 | 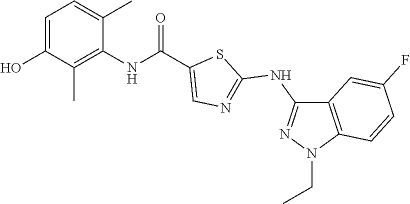 [SMILES: CCn1nc(Nc2ncc(C(=O)Nc3c(C)ccc(O)c3C)s2)c2cc(F)ccc21] |
| BAA- 460 | 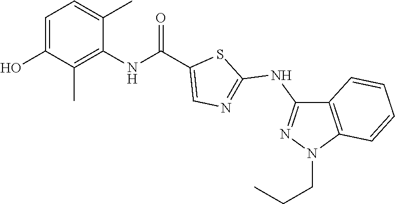 [SMILES: CCCn1nc(Nc2ncc(C(=O)Nc3c(C)ccc(O)c3C)s2)c2ccccc21] |
| BAA- 461 | 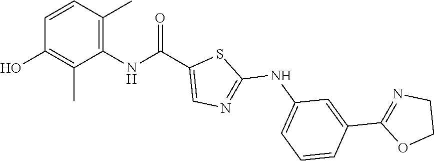 [SMILES: Cc1ccc(O)c(C)c1NC(=O)c1cnc(Nc2cccc(C3=NCCO3)c2)s1] |
| BAA- 462 | 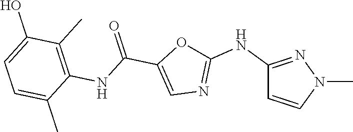 [SMILES: Cc1ccc(O)c(C)c1NC(=O)c1cnc(Nc2ccn(C)n2)o1] |
| BAA- 463 | 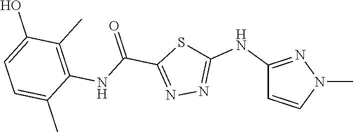 [SMILES: Cc1ccc(O)c(C)c1NC(=O)c1nnc(Nc2ccn(C)n2)s1] |
| BAA- 464 | 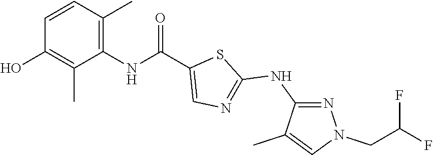 [SMILES: Cc1cn(CC(F)F)nc1Nc1ncc(C(=O)Nc2c(C)ccc(O)c2C)s1] |
| BAA- 465 | 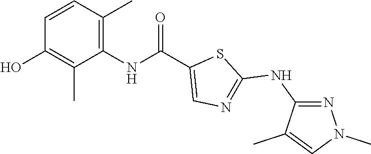 [SMILES: Cc1cn(C)nc1Nc1ncc(C(=O)Nc2c(C)ccc(O)c2C)s1] |
| BAA- 466 | 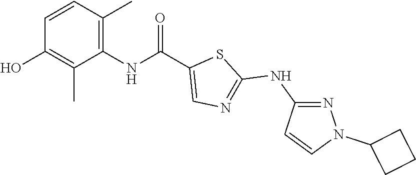 [SMILES: Cc1ccc(O)c(C)c1NC(=O)c1cnc(Nc2ccn(C3CCC3)n2)s1] |
| BAA- 467 | 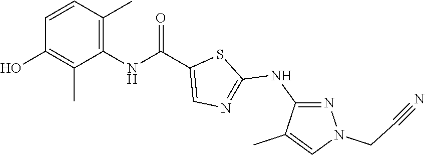 [SMILES: Cc1cn(CC#N)nc1Nc1ncc(C(=O)Nc2c(C)ccc(O)c2C)s1] |
| BAA- 468 | 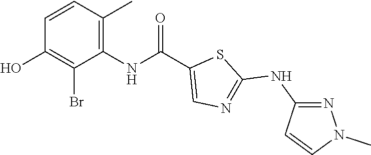 [SMILES: Cc1ccc(O)c(Br)c1NC(=O)c1cnc(Nc2ccn(C)n2)s1] |
| BAA- 469 | 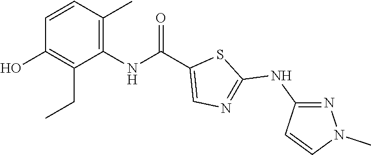 [SMILES: CCc1c(O)ccc(C)c1NC(=O)c1cnc(Nc2ccn(C)n2)s1] |
| BAA- 470 | 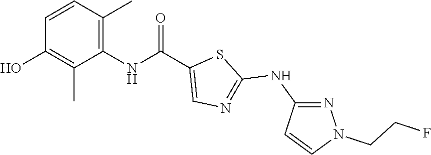 [SMILES: Cc1ccc(O)c(C)c1NC(=O)c1cnc(Nc2ccn(CCF)n2)s1] |
| BAA- 471 | 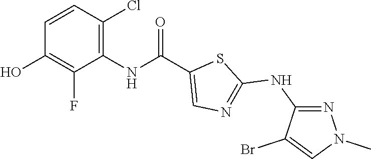 [SMILES: Cn1cc(Br)c(Nc2ncc(C(=O)Nc3c(Cl)ccc(O)c3F)s2)n1] |
| BAA- 472 | 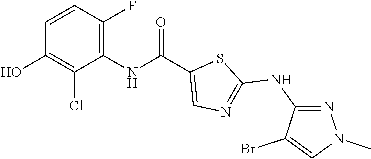 [SMILES: Cn1cc(Br)c(Nc2ncc(C(=O)Nc3c(F)ccc(O)c3Cl)s2)n1] |
| BAA- 473 | 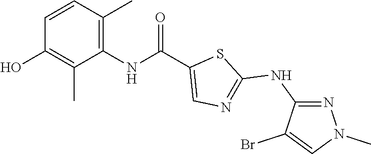 [SMILES: Cc1ccc(O)c(C)c1NC(=O)c1cnc(Nc2nn(C)cc2Br)s1] |
| BAA- 474 | 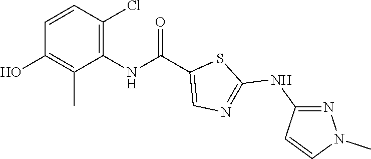 [SMILES: Cc1c(O)ccc(Cl)c1NC(=O)c1cnc(Nc2ccn(C)n2)s1] |
| BAA- 475 | 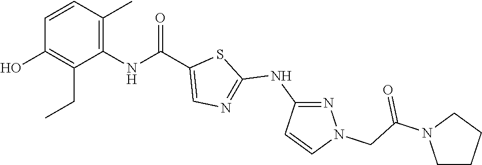 [SMILES: CCc1c(O)ccc(C)c1NC(=O)c1cnc(Nc2ccn(CC(=O)N3CCCC3)n2)s1] |
| BAA- 476 | 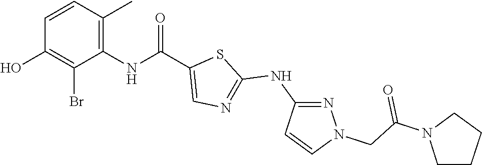 [SMILES: Cc1ccc(O)c(Br)c1NC(=O)c1cnc(Nc2ccn(CC(=O)N3CCCC3)n2)s1] |
| BAA- 477 | 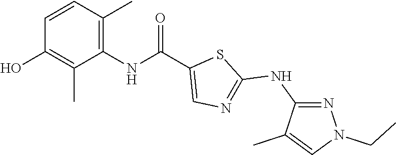 [SMILES: CCn1cc(C)c(Nc2ncc(C(=O)Nc3c(C)ccc(O)c3C)s2)n1] |
| BAA- 478 | 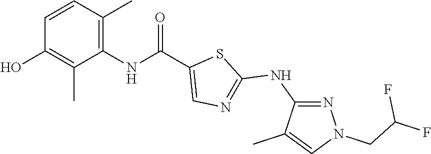 [SMILES: Cc1cn(CC(F)F)nc1Nc1ncc(C(=O)Nc2c(C)ccc(O)c2C)s1] |
| BAA- 479 | 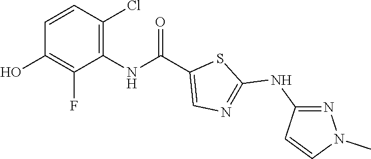 [SMILES: Cn1ccc(Nc2ncc(C(=O)Nc3c(Cl)ccc(O)c3F)s2)n1] |
| BAA- 480 | 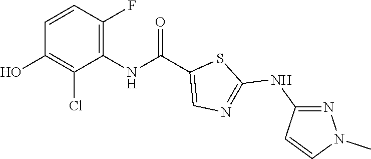 [SMILES: Cn1ccc(Nc2ncc(C(=O)Nc3c(F)ccc(O)c3Cl)s2)n1] |
| BAA- 481 | 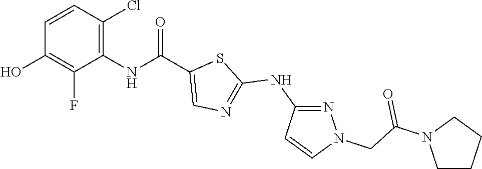 [SMILES: O=C(Nc1c(Cl)ccc(O)c1F)c1cnc(Nc2ccn(CC(=O)N3CCCC3)n2)s1] |
| BAA- 482 | 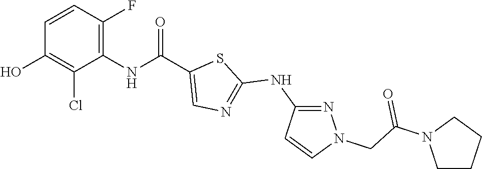 [SMILES: O=C(Nc1c(F)ccc(O)c1Cl)c1cnc(Nc2ccn(CC(=O)N3CCCC3)n2)s1] |
| BAA- 483 | 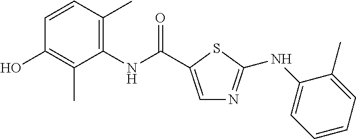 [SMILES: Cc1ccccc1Nc1ncc(C(=O)Nc2c(C)ccc(O)c2C)s1] |
| BAA- 484 | 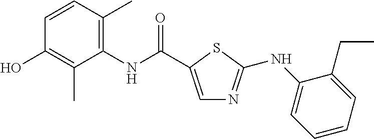 [SMILES: CCc1ccccc1Nc1ncc(C(=O)Nc2c(C)ccc(O)c2C)s1] |
| BAA- 485 | 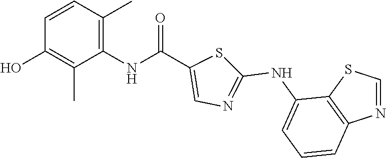 [SMILES: Cc1ccc(O)c(C)c1NC(=O)c1cnc(Nc2cccc3ncsc23)s1] |
| BAA- 486 | 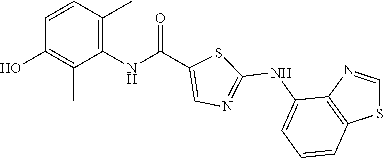 [SMILES: Cc1ccc(O)c(C)c1NC(=O)c1cnc(Nc2cccc3scnc23)s1] |
| BAA- 487 | 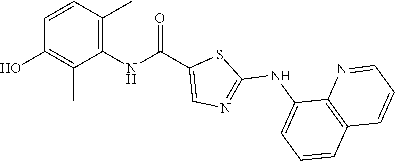 [SMILES: Cc1ccc(O)c(C)c1NC(=O)c1cnc(Nc2cccc3cccnc23)s1] |
| BAA- 488 | 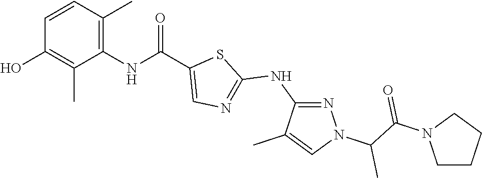 [SMILES: Cc1cn(C(C)C(=O)N2CCCC2)nc1Nc1ncc(C(=O)Nc2c(C)ccc(O)c2C)s1] |
| BAA- 489 | 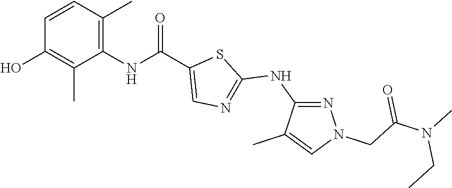 [SMILES: CCN(C)C(=O)Cn1cc(C)c(Nc2ncc(C(=O)Nc3c(C)ccc(O)c3C)s2)n1] |
| BAA- 490 | 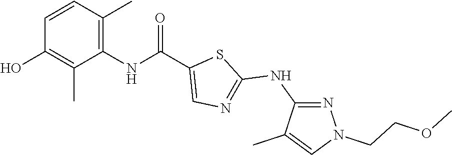 [SMILES: COCCn1cc(C)c(Nc2ncc(C(=O)Nc3c(C)ccc(O)c3C)s2)n1] |
| BAA- 491 | 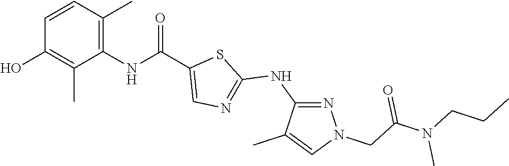 [SMILES: CCCN(C)C(=O)Cn1cc(C)c(Nc2ncc(C(=O)Nc3c(C)ccc(O)c3C)s2)n1] |
| BAA- 492 | 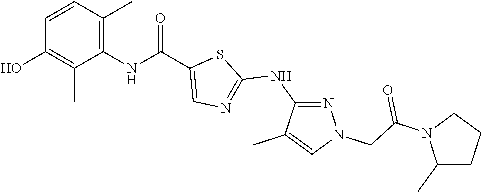 [SMILES: Cc1cn(CC(=O)N2CCCC2C)nc1Nc1ncc(C(=O)Nc2c(C)ccc(O)c2C)s1] |
| BAA- 493 | 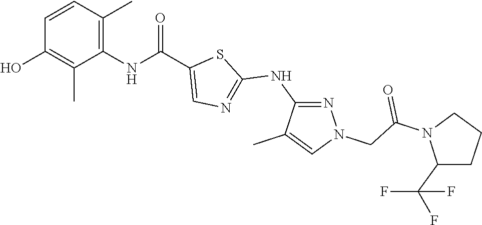 [SMILES: Cc1cn(CC(=O)N2CCCC2C(F)(F)F)nc1Nc1ncc(C(=O)Nc2c(C)ccc(O)c2C)s1] |
| BAA- 494 | 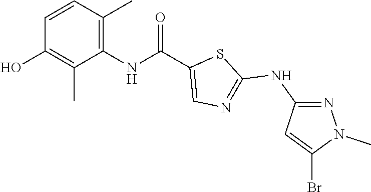 [SMILES: Cc1ccc(O)c(C)c1NC(=O)c1cnc(Nc2cc(Br)n(C)n2)s1] |
| BAA- 495 | 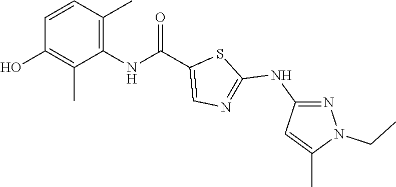 [SMILES: CCn1nc(Nc2ncc(C(=O)Nc3c(C)ccc(O)c3C)s2)cc1C] |
| BAA- 496 | 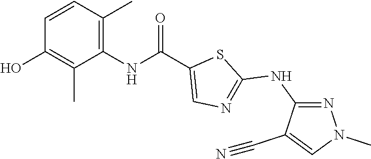 [SMILES: Cc1ccc(O)c(C)c1NC(=O)c1cnc(Nc2nn(C)cc2C#N)s1] |
| BAA- 497 | 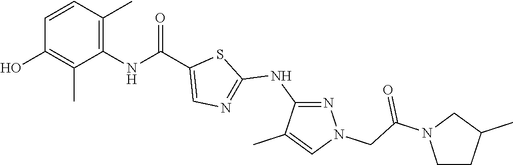 [SMILES: Cc1cn(CC(=O)N2CCC(C)C2)nc1Nc1ncc(C(=O)Nc2c(C)ccc(O)c2C)s1] |
| BAA- 498 | 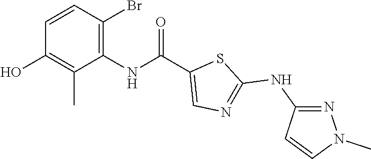 [SMILES: Cc1c(O)ccc(Br)c1NC(=O)c1cnc(Nc2ccn(C)n2)s1] |
| BAA- 499 | 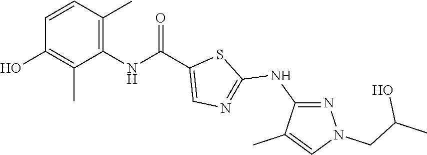 [SMILES: Cc1cn(CC(C)O)nc1Nc1ncc(C(=O)Nc2c(C)ccc(O)c2C)s1] |
| BAA- 500 | 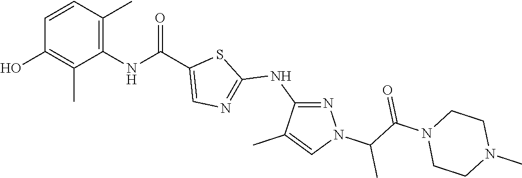 [SMILES: Cc1cn(C(C)C(=O)N2CCN(C)CC2)nc1Nc1ncc(C(=O)Nc2c(C)ccc(O)c2C)s1] |
| BAA- 501 | 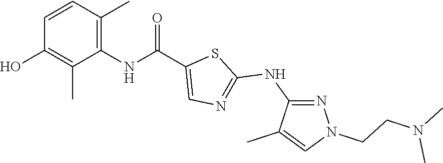 [SMILES: Cc1cn(CCN(C)C)nc1Nc1ncc(C(=O)Nc2c(C)ccc(O)c2C)s1] |
| BAA- 502 | 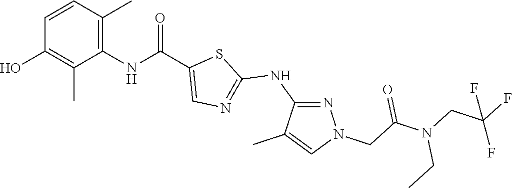 [SMILES: CCN(CC(F)(F)F)C(=O)Cn1cc(C)c(Nc2ncc(C(=O)Nc3c(C)ccc(O)c3C)s2)n1] |
| BAA- 503 | 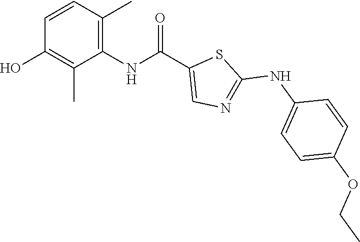 [SMILES: CCOc1ccc(Nc2ncc(C(=O)Nc3c(C)ccc(O)c3C)s2)cc1] |
| BAA- 504 | 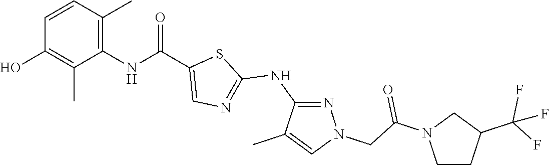 [SMILES: Cc1cn(CC(=O)N2CCC(C(F)(F)F)C2)nc1Nc1ncc(C(=O)Nc2c(C)ccc(O)c2C)s1] |
| BAA- 505 | 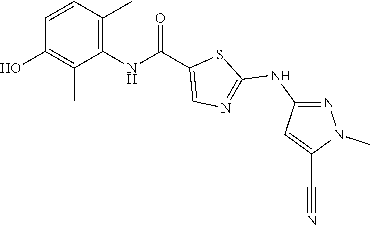 [SMILES: Cc1ccc(O)c(C)c1NC(=O)c1cnc(Nc2cc(C#N)n(C)n2)s1] |
| BAA- 506 | 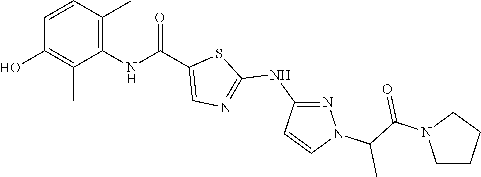 [SMILES: Cc1ccc(O)c(C)c1NC(=O)c1cnc(Nc2ccn(C(C)C(=O)N3CCCC3)n2)s1] |
| BAA- 507 | 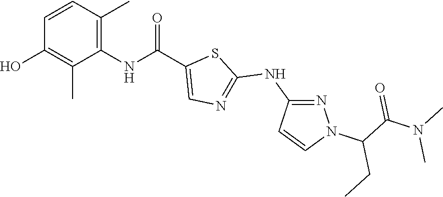 [SMILES: CCC(C(=O)N(C)C)n1ccc(Nc2ncc(C(=O)Nc3c(C)ccc(O)c3C)s2)n1] |
| BAA- 508 | 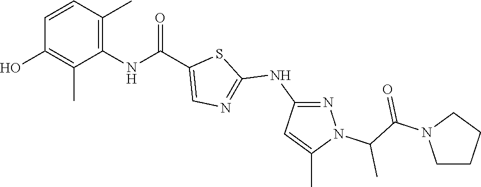 [SMILES: Cc1ccc(O)c(C)c1NC(=O)c1cnc(Nc2cc(C)n(C(C)C(=O)N3CCCC3)n2)s1] |
| BAA- 509 | 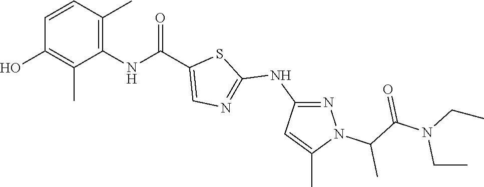 [SMILES: CCN(CC)C(=O)C(C)n1nc(Nc2ncc(C(=O)Nc3c(C)ccc(O)c3C)s2)cc1C] |
| BAA- 510 | 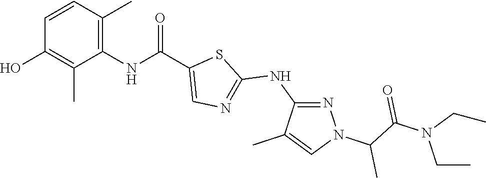 [SMILES: CCN(CC)C(=O)C(C)n1cc(C)c(Nc2ncc(C(=O)Nc3c(C)ccc(O)c3C)s2)n1] |
| BAA- 511 | 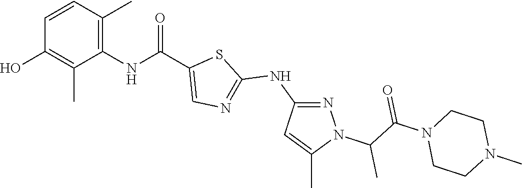 [SMILES: Cc1ccc(O)c(C)c1NC(=O)c1cnc(Nc2cc(C)n(C(C)C(=O)N3CCN(C)CC3)n2)s1] |
| BAA- 512 | 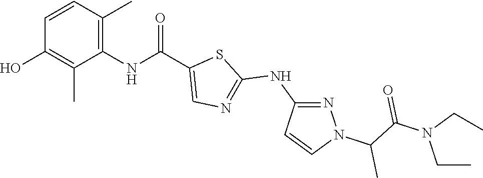 [SMILES: CCN(CC)C(=O)C(C)n1ccc(Nc2ncc(C(=O)Nc3c(C)ccc(O)c3C)s2)n1] |
| BAA- 513 | 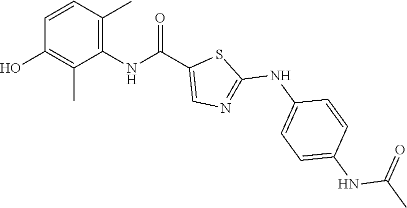 [SMILES: CC(=O)Nc1ccc(Nc2ncc(C(=O)Nc3c(C)ccc(O)c3C)s2)cc1] |
| BAA- 514 | 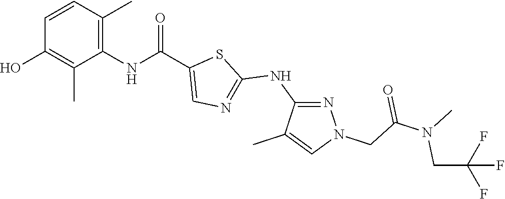 [SMILES: Cc1cn(CC(=O)N(C)CC(F)(F)F)nc1Nc1ncc(C(=O)Nc2c(C)ccc(O)c2C)s1] |
| BAA- 515 | 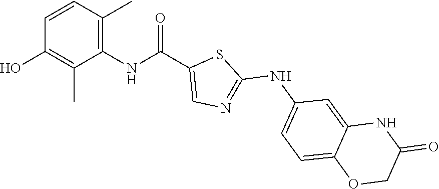 [SMILES: Cc1ccc(O)c(C)c1NC(=O)c1cnc(Nc2ccc3c(c2)NC(=O)CO3)s1] |
| BAA- 516 | 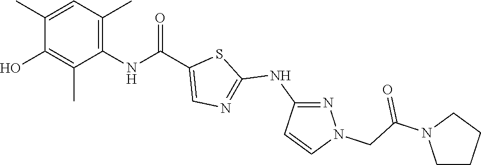 [SMILES: Cc1cc(C)c(NC(=O)c2cnc(Nc3ccn(CC(=O)N4CCCC4)n3)s2)c(C)c1O] |
| BAA- 517 | 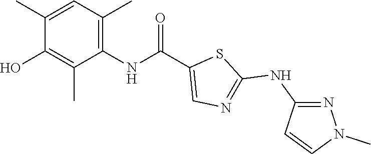 [SMILES: Cc1cc(C)c(NC(=O)c2cnc(Nc3ccn(C)n3)s2)c(C)c1O] |
| BAA- 518 | 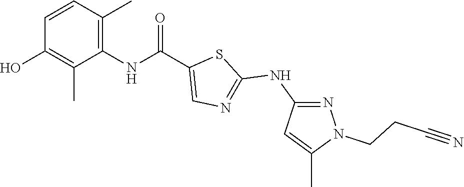 [SMILES: Cc1ccc(O)c(C)c1NC(=O)c1cnc(Nc2cc(C)n(CCC#N)n2)s1] |
| BAA- 519 | 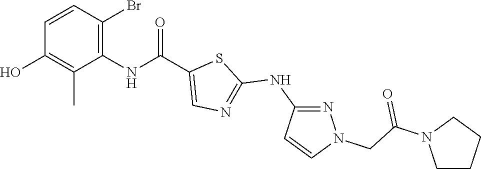 [SMILES: Cc1c(O)ccc(Br)c1NC(=O)c1cnc(Nc2ccn(CC(=O)N3CCCC3)n2)s1] |
| BAA- 520 | 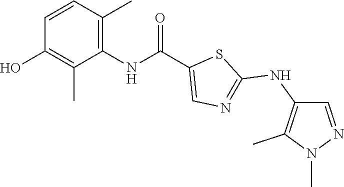 [SMILES: Cc1ccc(O)c(C)c1NC(=O)c1cnc(Nc2cnn(C)c2C)s1] |
| BAA- 521 | 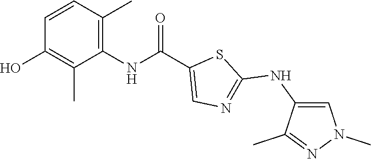 [SMILES: Cc1ccc(O)c(C)c1NC(=O)c1cnc(Nc2cn(C)nc2C)s1] |
| BAA- 522 | 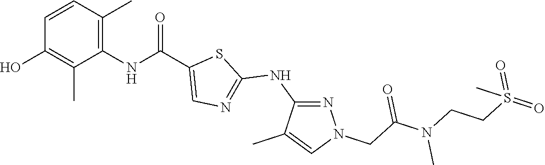 [SMILES: Cc1cn(CC(=O)N(C)CCS(C)(=O)=O)nc1Nc1ncc(C(=O)Nc2c(C)ccc(O)c2C)s1] |
| BAA- 523 | 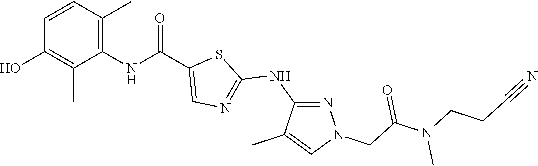 [SMILES: Cc1cn(CC(=O)N(C)CCC#N)nc1Nc1ncc(C(=O)Nc2c(C)ccc(O)c2C)s1] |
| BAA- 524 | 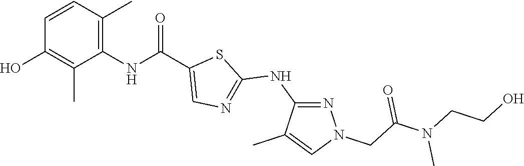 [SMILES: Cc1cn(CC(=O)N(C)CCO)nc1Nc1ncc(C(=O)Nc2c(C)ccc(O)c2C)s1] |
| BAA- 525 | 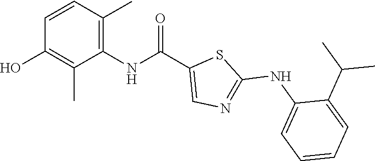 [SMILES: Cc1ccc(O)c(C)c1NC(=O)c1cnc(Nc2ccccc2C(C)C)s1] |
| BAA- 526 | 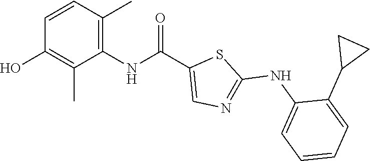 [SMILES: Cc1ccc(O)c(C)c1NC(=O)c1cnc(Nc2ccccc2C2CC2)s1] |
| BAA- 527 | 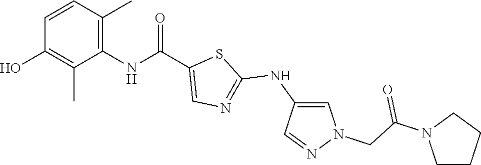 [SMILES: Cc1ccc(O)c(C)c1NC(=O)c1cnc(Nc2cnn(CC(=O)N3CCCC3)c2)s1] |
| BAA- 528 | 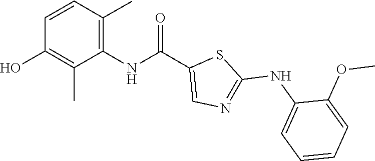 [SMILES: COc1ccccc1Nc1ncc(C(=O)Nc2c(C)ccc(O)c2C)s1] |
| BAA- 529 | 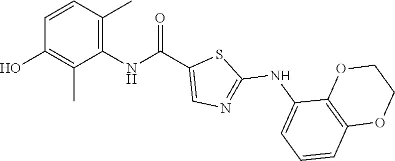 [SMILES: Cc1ccc(O)c(C)c1NC(=O)c1cnc(Nc2cccc3c2OCCO3)s1] |
| BAA- 530 | 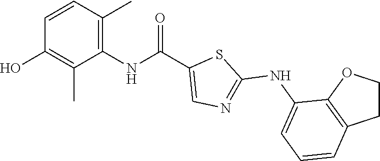 [SMILES: Cc1ccc(O)c(C)c1NC(=O)c1cnc(Nc2cccc3c2OCC3)s1] |
| BAA- 531 | 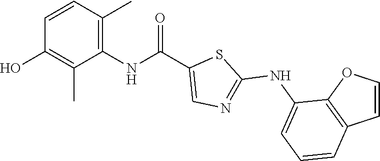 [SMILES: Cc1ccc(O)c(C)c1NC(=O)c1cnc(Nc2cccc3ccoc23)s1] |
| BAA- 532 | 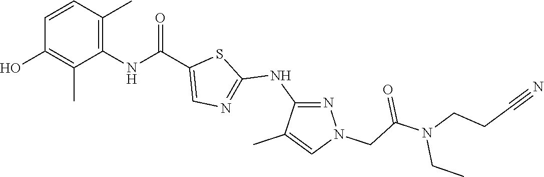 [SMILES: CCN(CCC#N)C(=O)Cn1cc(C)c(Nc2ncc(C(=O)Nc3c(C)ccc(O)c3C)s2)n1] |
| BAA- 533 | 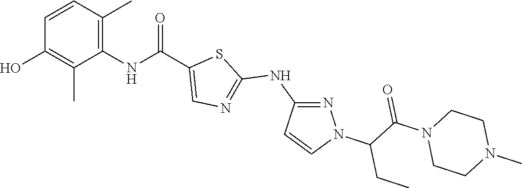 [SMILES: CCC(C(=O)N1CCN(C)CC1)n1ccc(Nc2ncc(C(=O)Nc3c(C)ccc(O)c3C)s2)n1] |
| BAA- 534 | 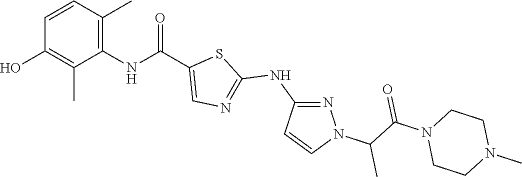 [SMILES: Cc1ccc(O)c(C)c1NC(=O)c1cnc(Nc2ccn(C(C)C(=O)N3CCN(C)CC3)n2)s1] |
| BAA- 535 | 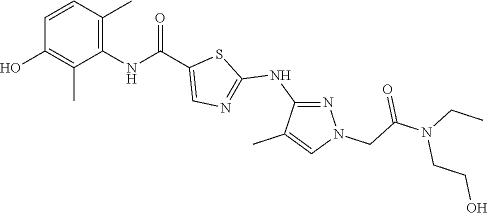 [SMILES: CCN(CCO)C(=O)Cn1cc(C)c(Nc2ncc(C(=O)Nc3c(C)ccc(O)c3C)s2)n1] |
| BAA- 536 | 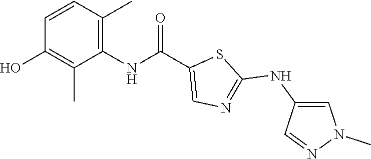 [SMILES: Cc1ccc(O)c(C)c1NC(=O)c1cnc(Nc2cnn(C)c2)s1] |
| BAA- 537 | 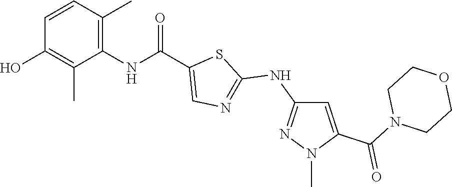 [SMILES: Cc1ccc(O)c(C)c1NC(=O)c1cnc(Nc2cc(C(=O)N3CCOCC3)n(C)n2)s1] |
| BAA- 538 | 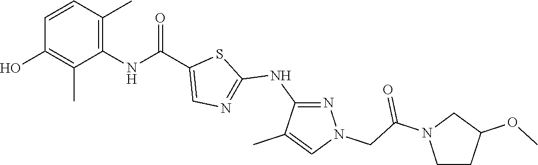 [SMILES: COC1CCN(C(=O)Cn2cc(C)c(Nc3ncc(C(=O)Nc4c(C)ccc(O)c4C)s3)n2)C1] |
| BAA- 539 | 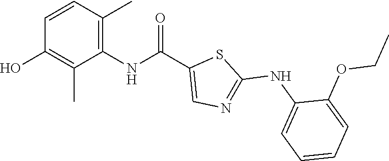 [SMILES: CCOc1ccccc1Nc1ncc(C(=O)Nc2c(C)ccc(O)c2C)s1] |
| BAA- 540 | 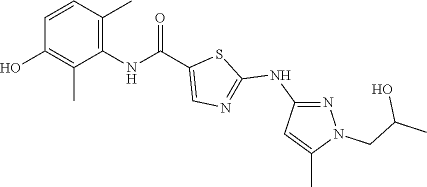 [SMILES: Cc1ccc(O)c(C)c1NC(=O)c1cnc(Nc2cc(C)n(CC(C)O)n2)s1] |
| BAA- 541 | 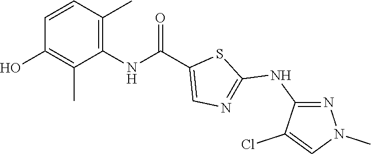 [SMILES: Cc1ccc(O)c(C)c1NC(=O)c1cnc(Nc2nn(C)cc2Cl)s1] |
| BAA- 542 | 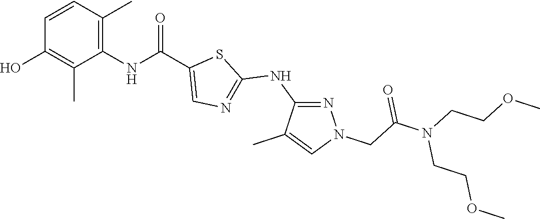 [SMILES: COCCN(CCOC)C(=O)Cn1cc(C)c(Nc2ncc(C(=O)Nc3c(C)ccc(O)c3C)s2)n1] |
| BAA- 543 | 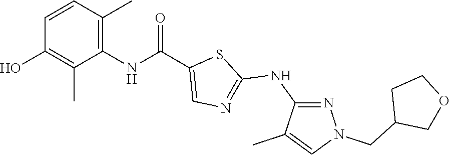 [SMILES: Cc1cn(CC2CCOC2)nc1Nc1ncc(C(=O)Nc2c(C)ccc(O)c2C)s1] |
| BAA- 544 | 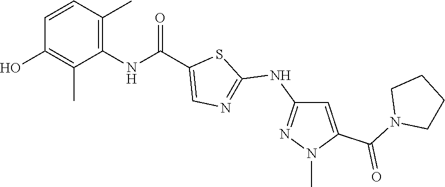 [SMILES: Cc1ccc(O)c(C)c1NC(=O)c1cnc(Nc2cc(C(=O)N3CCCC3)n(C)n2)s1] |
| BAA- 545 | 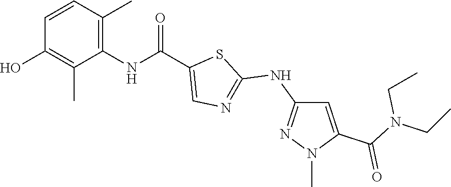 [SMILES: CCN(CC)C(=O)c1cc(Nc2ncc(C(=O)Nc3c(C)ccc(O)c3C)s2)nn1C] |
| BAA- 546 | 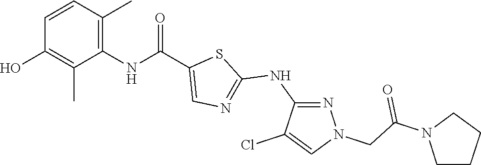 [SMILES: Cc1ccc(O)c(C)c1NC(=O)c1cnc(Nc2nn(CC(=O)N3CCCC3)cc2Cl)s1] |
| BAA- 547 | 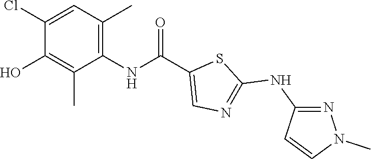 [SMILES: Cc1cc(Cl)c(O)c(C)c1NC(=O)c1cnc(Nc2ccn(C)n2)s1] |
| BAA- 548 | 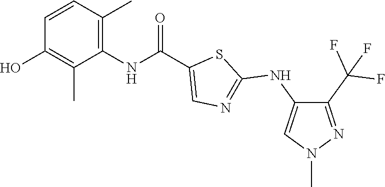 [SMILES: Cc1ccc(O)c(C)c1NC(=O)c1cnc(Nc2cn(C)nc2C(F)(F)F)s1] |
| BAA- 549 | 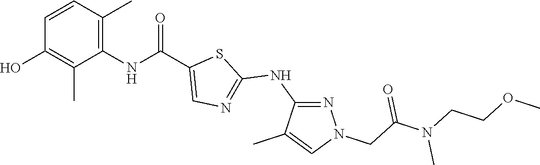 [SMILES: COCCN(C)C(=O)Cn1cc(C)c(Nc2ncc(C(=O)Nc3c(C)ccc(O)c3C)s2)n1] |
| BAA- 550 | 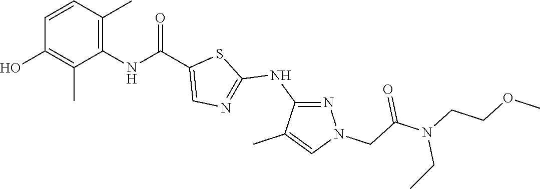 [SMILES: CCN(CCOC)C(=O)Cn1cc(C)c(Nc2ncc(C(=O)Nc3c(C)ccc(O)c3C)s2)n1] |
| BAA- 551 | 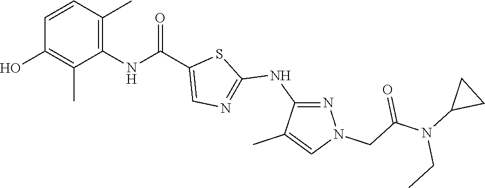 [SMILES: CCN(C(=O)Cn1cc(C)c(Nc2ncc(C(=O)Nc3c(C)ccc(O)c3C)s2)n1)C1CC1] |
| BAA- 552 | 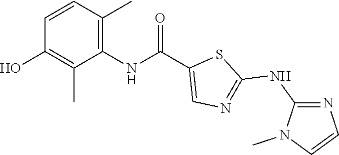 [SMILES: Cc1ccc(O)c(C)c1NC(=O)c1cnc(Nc2nccn2C)s1] |
| BAA- 553 | 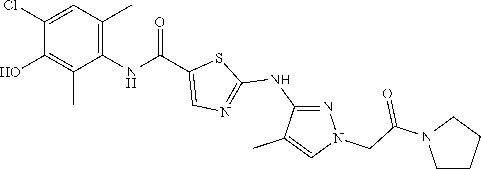 [SMILES: Cc1cn(CC(=O)N2CCCC2)nc1Nc1ncc(C(=O)Nc2c(C)cc(Cl)c(O)c2C)s1] |
| BAA- 554 | 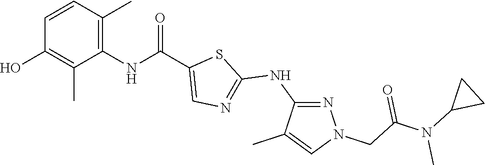 [SMILES: Cc1cn(CC(=O)N(C)C2CC2)nc1Nc1ncc(C(=O)Nc2c(C)ccc(O)c2C)s1] |
| BAA- 555 | 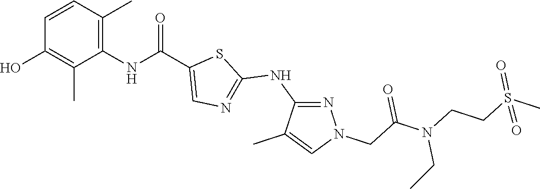 [SMILES: CCN(CCS(C)(=O)=O)C(=O)Cn1cc(C)c(Nc2ncc(C(=O)Nc3c(C)ccc(O)c3C)s2)n1] |
| BAA- 556 | 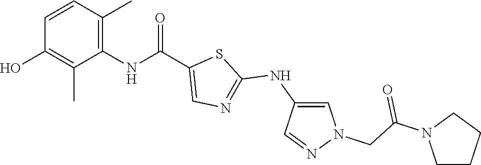 [SMILES: Cc1ccc(O)c(C)c1NC(=O)c1cnc(Nc2cnn(CC(=O)N3CCCC3)c2)s1] |
| BAA- 557 | 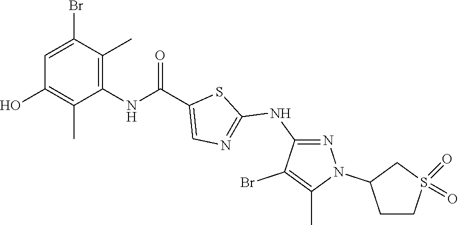 [SMILES: Cc1c(O)cc(Br)c(C)c1NC(=O)c1cnc(Nc2nn(C3CCS(=O)(=O)C3)c(C)c2Br)s1] |
| BAA- 558 | 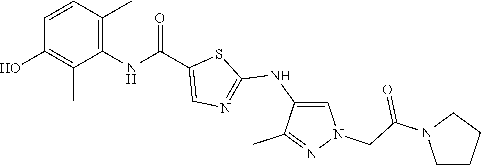 [SMILES: Cc1ccc(O)c(C)c1NC(=O)c1cnc(Nc2cn(CC(=O)N3CCCC3)nc2C)s1] |
| BAA- 559 | 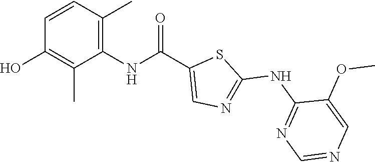 [SMILES: COc1cncnc1Nc1ncc(C(=O)Nc2c(C)ccc(O)c2C)s1] |
| BAA- 560 | 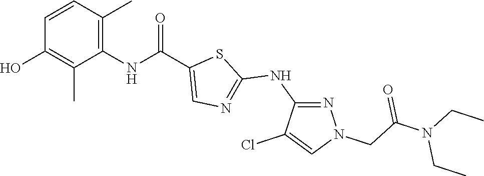 [SMILES: CCN(CC)C(=O)Cn1cc(Cl)c(Nc2ncc(C(=O)Nc3c(C)ccc(O)c3C)s2)n1] |
| BAA- 561 | 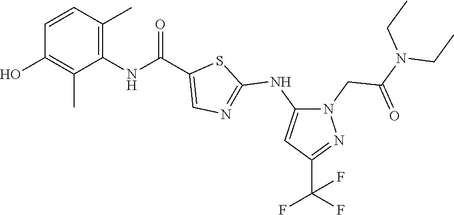 [SMILES: CCN(CC)C(=O)Cn1nc(C(F)(F)F)cc1Nc1ncc(C(=O)Nc2c(C)ccc(O)c2C)s1] |
| BAA- 562 | 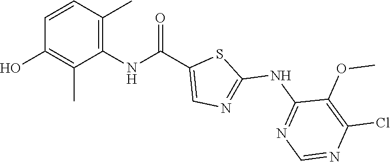 [SMILES: COc1c(Cl)ncnc1Nc1ncc(C(=O)Nc2c(C)ccc(O)c2C)s1] |
| BAA- 563 | 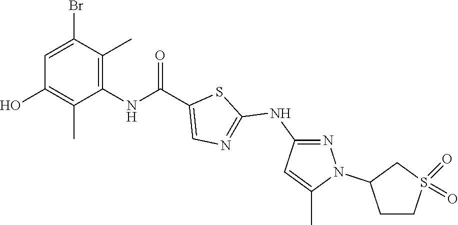 [SMILES: Cc1c(O)cc(Br)c(C)c1NC(=O)c1cnc(Nc2cc(C)n(C3CCS(=O)(=O)C3)n2)s1] |
| BAA- 564 | 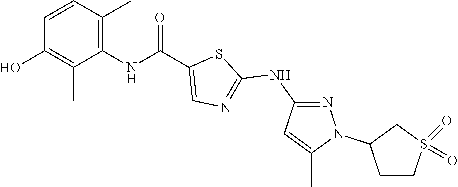 [SMILES: Cc1ccc(O)c(C)c1NC(=O)c1cnc(Nc2cc(C)n(C3CCS(=O)(=O)C3)n2)s1] |
| BAA- 565 | 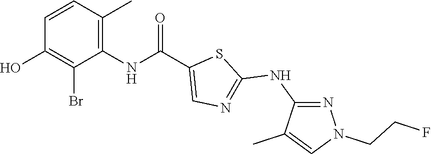 [SMILES: Cc1cn(CCF)nc1Nc1ncc(C(=O)Nc2c(C)ccc(O)c2Br)s1] |
| BAA- 566 | 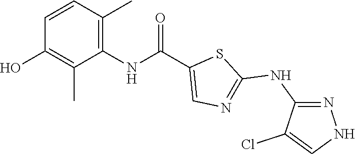 [SMILES: Cc1ccc(O)c(C)c1NC(=O)c1cnc(Nc2n[nH]cc2Cl)s1] |
| BAA- 567 | 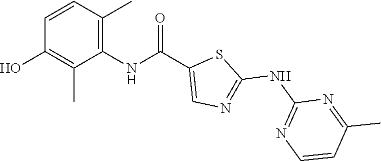 [SMILES: Cc1ccnc(Nc2ncc(C(=O)Nc3c(C)ccc(O)c3C)s2)n1] |
| BAA- 568 | 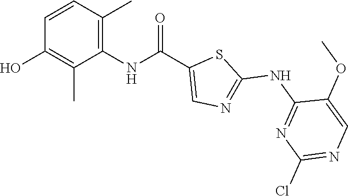 [SMILES: COc1cnc(Cl)nc1Nc1ncc(C(=O)Nc2c(C)ccc(O)c2C)s1] |
| BAA- 569 | 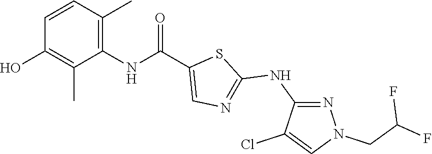 [SMILES: Cc1ccc(O)c(C)c1NC(=O)c1cnc(Nc2nn(CC(F)F)cc2Cl)s1] |
| BAA- 570 | 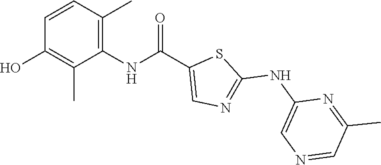 [SMILES: Cc1cncc(Nc2ncc(C(=O)Nc3c(C)ccc(O)c3C)s2)n1] |
| BAA- 571 | 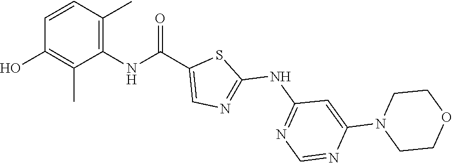 [SMILES: Cc1ccc(O)c(C)c1NC(=O)c1cnc(Nc2cc(N3CCOCC3)ncn2)s1] |
| BAA- 572 | 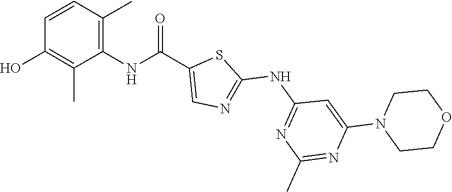 [SMILES: Cc1nc(Nc2ncc(C(=O)Nc3c(C)ccc(O)c3C)s2)cc(N2CCOCC2)n1] |
| BAA- 573 | 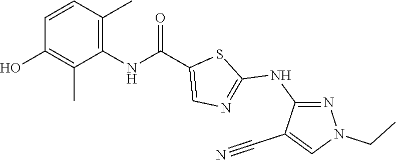 [SMILES: CCn1cc(C#N)c(Nc2ncc(C(=O)Nc3c(C)ccc(O)c3C)s2)n1] |
| BAA- 574 | 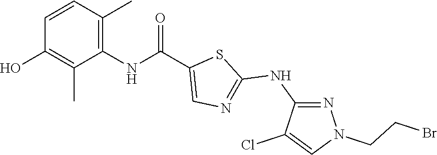 [SMILES: Cc1ccc(O)c(C)c1NC(=O)c1cnc(Nc2nn(CCBr)cc2Cl)s1] |
| BAA- 575 | 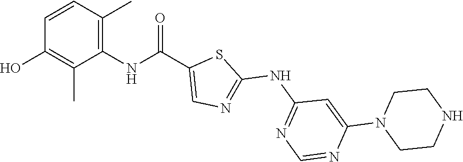 [SMILES: Cc1ccc(O)c(C)c1NC(=O)c1cnc(Nc2cc(N3CCNCC3)ncn2)s1] |
| BAA- 576 | 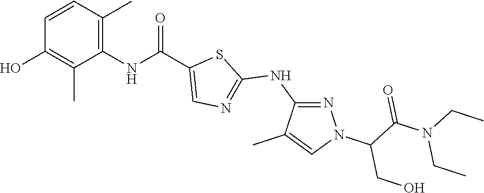 [SMILES: CCN(CC)C(=O)C(CO)n1cc(C)c(Nc2ncc(C(=O)Nc3c(C)ccc(O)c3C)s2)n1] |
| BAA- 577 | 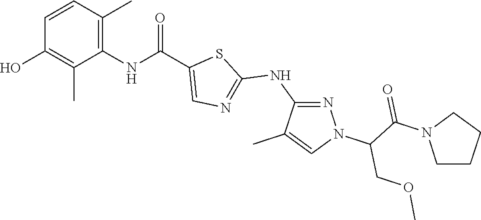 [SMILES: COCC(C(=O)N1CCCC1)n1cc(C)c(Nc2ncc(C(=O)Nc3c(C)ccc(O)c3C)s2)n1] |
| BAA- 578 | 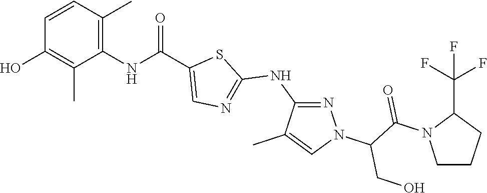 [SMILES: Cc1cn(C(CO)C(=O)N2CCCC2C(F)(F)F)nc1Nc1ncc(C(=O)Nc2c(C)ccc(O)c2C)s1] |
| BAA- 579 | 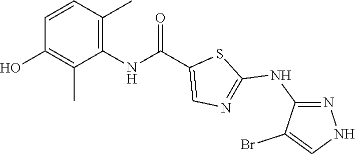 [SMILES: Cc1ccc(O)c(C)c1NC(=O)c1cnc(Nc2n[nH]cc2Br)s1] |
| BAA- 580 | 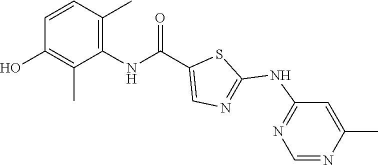 [SMILES: Cc1cc(Nc2ncc(C(=O)Nc3c(C)ccc(O)c3C)s2)ncn1] |
| BAA- 581 | 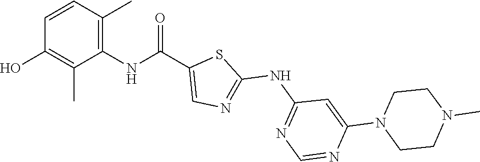 [SMILES: Cc1ccc(O)c(C)c1NC(=O)c1cnc(Nc2cc(N3CCN(C)CC3)ncn2)s1] |
| BAA- 582 | 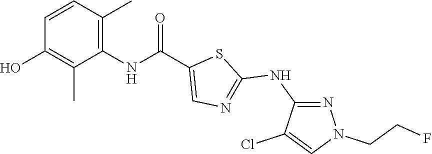 [SMILES: Cc1ccc(O)c(C)c1NC(=O)c1cnc(Nc2nn(CCF)cc2Cl)s1] |
| BAA- 583 | 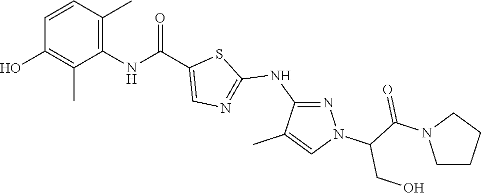 [SMILES: Cc1cn(C(CO)C(=O)N2CCCC2)nc1Nc1ncc(C(=O)Nc2c(C)ccc(O)c2C)s1] |
| BAA- 584 | 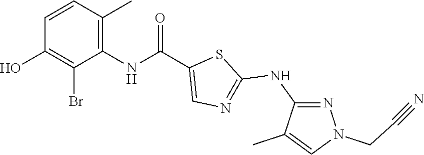 [SMILES: Cc1cn(CC#N)nc1Nc1ncc(C(=O)Nc2c(C)ccc(O)c2Br)s1] |
| BAA- 585 | 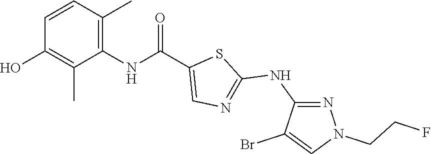 [SMILES: Cc1ccc(O)c(C)c1NC(=O)c1cnc(Nc2nn(CCF)cc2Br)s1] |
| BAA- 586 | 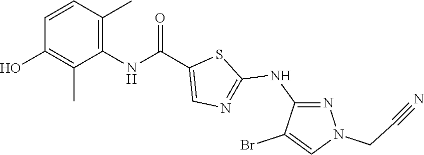 [SMILES: Cc1ccc(O)c(C)c1NC(=O)c1cnc(Nc2nn(CC#N)cc2Br)s1] |
| BAA- 587 | 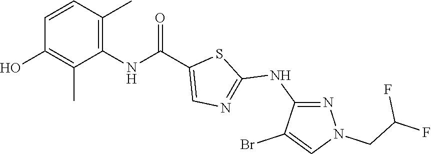 [SMILES: Cc1ccc(O)c(C)c1NC(=O)c1cnc(Nc2nn(CC(F)F)cc2Br)s1] |
| BAA- 588 | 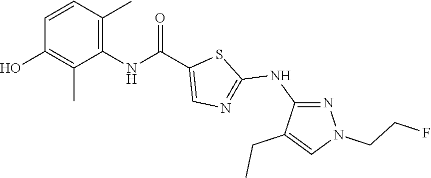 [SMILES: CCc1cn(CCF)nc1Nc1ncc(C(=O)Nc2c(C)ccc(O)c2C)s1] |
| BAA- 589 | 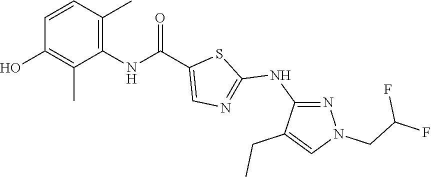 [SMILES: CCc1cn(CC(F)F)nc1Nc1ncc(C(=O)Nc2c(C)ccc(O)c2C)s1] |
| BAA- 590 | 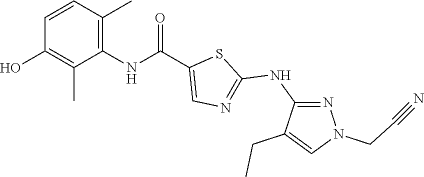 [SMILES: CCc1cn(CC#N)nc1Nc1ncc(C(=O)Nc2c(C)ccc(O)c2C)s1] |
| BAA- 591 | 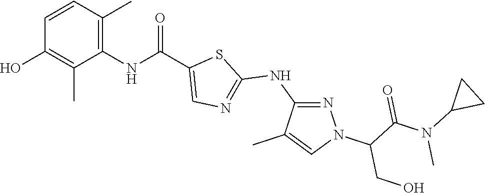 [SMILES: Cc1cn(C(CO)C(=O)N(C)C2CC2)nc1Nc1ncc(C(=O)Nc2c(C)ccc(O)c2C)s1] |
| BAA- 592 | 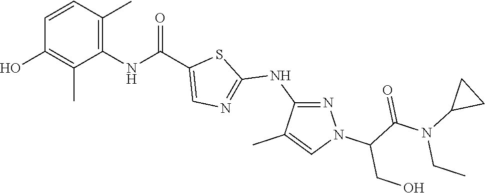 [SMILES: CCN(C(=O)C(CO)n1cc(C)c(Nc2ncc(C(=O)Nc3c(C)ccc(O)c3C)s2)n1)C1CC1] |
| BAA- 593 | 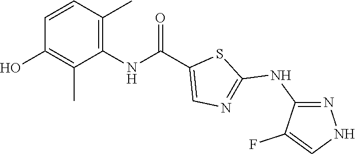 [SMILES: Cc1ccc(O)c(C)c1NC(=O)c1cnc(Nc2n[nH]cc2F)s1] |
| BAA- 594 | 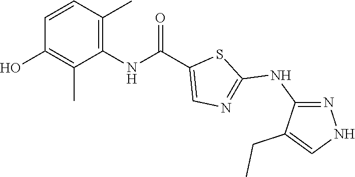 [SMILES: CCc1c[nH]nc1Nc1ncc(C(=O)Nc2c(C)ccc(O)c2C)s1] |
| BAA- 595 | 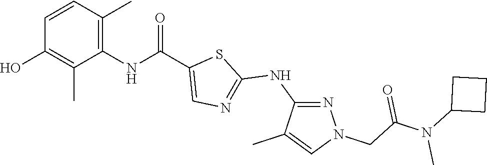 [SMILES: Cc1cn(CC(=O)N(C)C2CCC2)nc1Nc1ncc(C(=O)Nc2c(C)ccc(O)c2C)s1] |
| BAA- 596 | 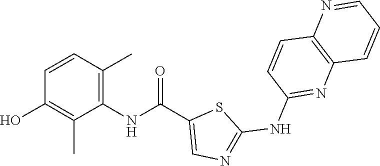 [SMILES: Cc1ccc(O)c(C)c1NC(=O)c1cnc(Nc2ccc3ncccc3n2)s1] |
| BAA- 597 | 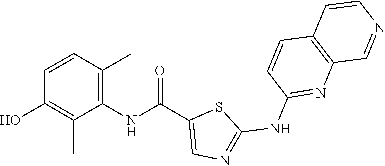 [SMILES: Cc1ccc(O)c(C)c1NC(=O)c1cnc(Nc2ccc3ccncc3n2)s1] |
| BAA- 598 | 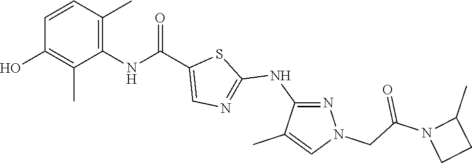 [SMILES: Cc1cn(CC(=O)N2CCC2C)nc1Nc1ncc(C(=O)Nc2c(C)ccc(O)c2C)s1] |
| BAA- 599 | 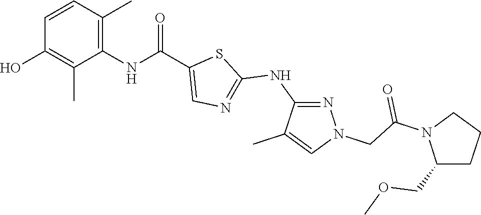 [SMILES: COC[C@H]1CCCN1C(=O)Cn1cc(C)c(Nc2ncc(C(=O)Nc3c(C)ccc(O)c3C)s2)n1] |
| BAA- 600 | 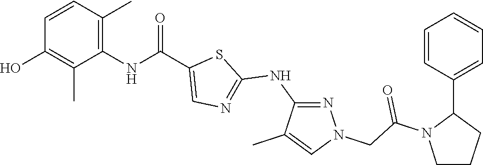 [SMILES: Cc1cn(CC(=O)N2CCCC2c2ccccc2)nc1Nc1ncc(C(=O)Nc2c(C)ccc(O)c2C)s1] |
| BAA- 601 | 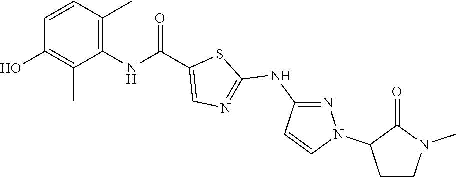 [SMILES: Cc1ccc(O)c(C)c1NC(=O)c1cnc(Nc2ccn(C3CCN(C)C3=O)n2)s1] |
| BAA- 602 | 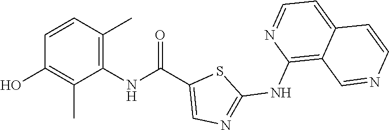 [SMILES: Cc1ccc(O)c(C)c1NC(=O)c1cnc(Nc2nccc3ccncc23)s1] |
| BAA- 603 | 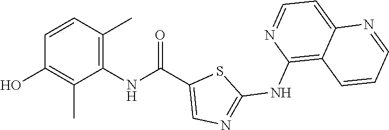 [SMILES: Cc1ccc(O)c(C)c1NC(=O)c1cnc(Nc2nccc3ncccc23)s1] |
| BAA- 604 | 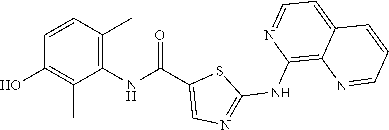 [SMILES: Cc1ccc(O)c(C)c1NC(=O)c1cnc(Nc2nccc3cccnc23)s1] |
| BAA- 605 | 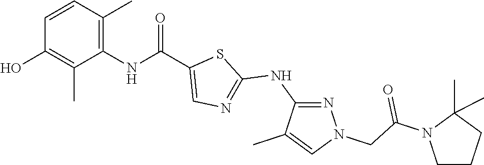 [SMILES: Cc1cn(CC(=O)N2CCCC2(C)C)nc1Nc1ncc(C(=O)Nc2c(C)ccc(O)c2C)s1] |
| BAA- 606 | 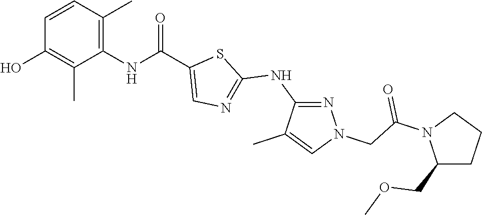 [SMILES: COC[C@@H]1CCCN1C(=O)Cn1cc(C)c(Nc2ncc(C(=O)Nc3c(C)ccc(O)c3C)s2)n1] |
| BAA- 607 | 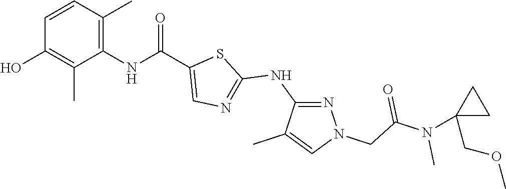 [SMILES: COCC1(N(C)C(=O)Cn2cc(C)c(Nc3ncc(C(=O)Nc4c(C)ccc(O)c4C)s3)n2)CC1] |
| BAA- 608 | 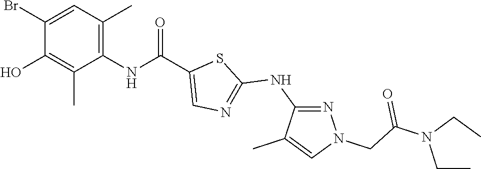 [SMILES: CCN(CC)C(=O)Cn1cc(C)c(Nc2ncc(C(=O)Nc3c(C)cc(Br)c(O)c3C)s2)n1] |
| BAA- 609 | 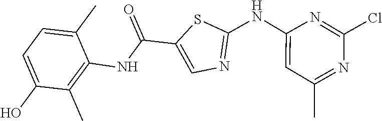 [SMILES: Cc1cc(Nc2ncc(C(=O)Nc3c(C)ccc(O)c3C)s2)nc(Cl)n1] |
| BAA- 610 | 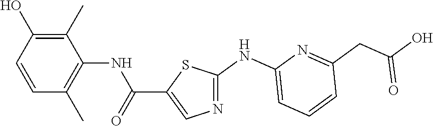 [SMILES: Cc1ccc(O)c(C)c1NC(=O)c1cnc(Nc2cccc(CC(=O)O)n2)s1] |

In one embodiment, the BAA compound is obtainable (or obtained) by following the methods described in the experimental section.

In one embodiment, the BAA compound is provided according to any embodiment described herein (for example, embodiment (1), (2) or (3); or claim 1) with the proviso that any of the specific Examples are individually disclaimed. For example, a further feature is any embodiment described herein (for example, embodiment (1), (2) or (3); or claim 1) with the proviso that any (for example any one, any two, or any three) of the compounds in the preceding table are individually disclaimed.

In one embodiment, the BAA compound is provided according to any embodiment described herein (for example, embodiment (1), (2) or (3); or claim 1) with the proviso that any other embodiment described herein is specifically disclaimed.

### Combinations

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the chemical groups represented by the variables (e.g., Ring A, Ring B, etc.) are specifically embraced by the present invention and are disclosed herein just as if each and every combination was individually and explicitly disclosed, to the extent that such combinations embrace compounds that are stable compounds (i.e., compounds that can be isolated, characterised, and tested for biological activity). In this context, the skilled person will readily appreciate that certain combinations of groups (e.g., substituents) may give rise to compounds which may not be readily synthesized and/or are chemically unstable. In addition, all sub-combinations of the chemical groups listed in the embodiments describing such variables are also specifically embraced by the present invention and are disclosed herein just as if each and every such sub-combination of chemical groups was individually and explicitly disclosed herein.

### Substantially Purified Forms

One aspect of the present invention pertains to BAA compounds, as described herein, in substantially purified form and/or in a form substantially free from contaminants.

In one embodiment, the substantially purified form is at least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless otherwise specified, the substantially purified form refers to the compound in any stereoisomeric or enantiomeric form. For example, in one embodiment, the substantially purified form refers to a mixture of stereoisomers, i.e., purified with respect to other compounds. In one embodiment, the substantially purified form refers to one stereoisomer, e.g., optically pure stereoisomer. In one embodiment, the substantially purified form refers to a mixture of enantiomers. In one embodiment, the substantially purified form refers to an equimolar mixture of enantiomers (i.e., a racemic mixture, a racemate). In one embodiment, the substantially purified form refers to one enantiomer, e.g., optically pure enantiomer.

In one embodiment, the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

Unless specified, the contaminants refer to other compounds, that is, other than stereoisomers or enantiomers. In one embodiment, the contaminants refer to other compounds and other stereoisomers. In one embodiment, the contaminants refer to other compounds and the other enantiomer.

In one embodiment, the substantially purified form is at least 60% optically pure (i.e., 60% of the compound, on a molar basis, is the desired stereoisomer or enantiomer, and 40% is the undesired stereoisomer or enantiomer), e.g., at least 70% optically pure, e.g., at least 80% optically pure, e.g., at least 90% optically pure, e.g., at least 95% optically pure, e.g., at least 97% optically pure, e.g., at least 98% optically pure, e.g., at least 99% optically pure.

Isomers Certain compounds may exist in one or more particular geometric, optical, enantiomeric, diastereoisomeric, epimeric, atropic, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

A reference to a class of structures may well include structurally isomeric forms falling within that class (e.g., C₁₋₆alkyl includes n-propyl and iso-propyl; butyl includes n-, iso-, sec-, and tert-butyl; methoxyphenyl includes ortho-, meta-, and para-methoxyphenyl).

However, reference to a specific group or substitution pattern is not intended to include other structural (or constitutional isomers) which differ with respect to the connections between atoms rather than by positions in space. For example, a reference to a methoxy group, —OCH₃, is not to be construed as a reference to its structural isomer, a hydroxymethyl group, —CH₂OH. Similarly, a reference specifically to ortho-chlorophenyl is not to be construed as a reference to its structural isomer, meta-chlorophenyl.

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro. A reference herein to one tautomer is intended to encompass both tautomers.

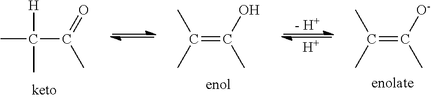

For example, 1H-pyridin-2-one-5-yl and 2-hydroxyl-pyridin-5-yl(shown below) are tautomers of one another. A reference herein to one is intended to encompass both.

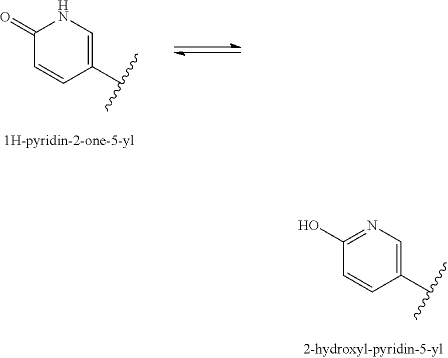 [SMILES: *c1ccc(=O)[nH]c1 ; *c1ccc(O)nc1]

Note that specifically included in the term “isomer” are compounds with one or more isotopic substitutions. For example, H may be in any isotopic form, including ¹H, ²H(D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the compound, for example, a pharmaceutically-acceptable salt. The term “salt” is used herein to refer to a solid complex comprising a first co-forming entity (e.g. a compound such as a BAA compound) and a second co-forming entity (e.g. a suitable Brønsted acid or base), where there is complete transfer of a proton from one entity to another. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group, which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺ as well as the ammonium ion (i.e., NH₄⁺). Examples of suitable organic cations include, but are not limited to substituted ammonium ions (e.g., NH_{3R}⁺, NH₂R₂⁺, NHR₃⁺, NR₄⁺), for example, where each R is independently linear or branched saturated C₁₋₁₈alkyl, C₃₋₈cycloalkyl, C₃₋₈cycloalkyl-C₁₋₆alkyl, and phenyl-C₁₋₆alkyl, wherein the phenyl group is optionally substituted. Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group, which upon protonation may become cationic (e.g., —NH₂ may become —NH₃⁺), then a salt may be formed with a suitable anion.

For example, if a parent structure contains a cationic group (e.g., —NMe₂⁺), or has a functional group, which upon protonation may become cationic (e.g., —NH₂ may become —NH₃⁺), then a salt may be formed with a suitable anion. In the case of a quaternary ammonium compound a counter-anion is generally always present in order to balance the positive charge. If, in addition to a cationic group (e.g., —NMe₂⁺, —NH₃⁺), the compound also contains a group capable of forming an anion (e.g., —COOH), then an inner salt (also referred to as a zwitterion) may be formed.

Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyloxybenzoic, acetic, trifluoroacetic, ascorbic, aspartic, benzoic, camphorsulfonic, cinnamic, citric, edetic, 1,2-ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Examples of suitable counter-ions which are especially suitable for quaternary ammonium compounds (e.g., those with a —NMe₂⁺group) include 1-adamantanesulfonate, benzenesulfonate, bisulfate, bromide, chloride, iodide, methanesulfonate, methylsulfate, 1,5-napthalene-bis-sulfonate, 4-nitrobenzenesulfonate, formate, tartrate, tosylate, trifluoroacetate, trifluoromethylsulfonate, sulphate. Again, if the compound also contains a group capable of forming an anion (e.g., —COOH), then an inner salt may be formed.

Unless otherwise specified, a reference to a particular compound also includes salt forms thereof.

In one embodiment, the BAA compound is provided in the form of a salt.

In one embodiment, the BAA compound is provided in a neutral form (for example as a free acid, free base, or zwitterion).

### Solvates and Hydrates

It may be convenient or desirable to prepare, purify, and/or handle a corresponding solvate of the compound. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate and hydrate forms thereof.

In one embodiment, the BAA compound is provided in the form of a solvate.

In one embodiment, the BAA compound is provided in the form of a hydrate.

In one embodiment, the BAA compound is provided in unsolvated form.

### Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, reactive chemical reagents, and the like). In practice, well-known chemical methods are employed to reversibly render unreactive a functional group, which otherwise would be reactive, under specified conditions. In a chemically protected form, one or more reactive functional groups are in the form of a protected or protecting group (alternatively as a masked or masking group or a blocked or blocking group). By protecting a reactive functional group, reactions involving other unprotected reactive functional groups can be performed, without affecting the protected group; the protecting group may be removed or the masking group transformed, usually in a subsequent step, without substantially affecting the remainder of the molecule. See, for example, Protective Groups in Organic Synthesis (T. Green and P. Wuts; 4th Edition; John Wiley and Sons, 2006).

A wide variety of such “protecting,” “blocking,” or “masking” methods are widely used and well known in organic synthesis. For example, a compound which has two non-equivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl(diphenylmethyl), or trityl(triphenylmethyl) ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a 1,1-diether (>C(OR)₂), by reaction with, for example, a primary alcohol in the presence of an acid. The aldehyde or ketone group is readily regenerated, for example, by hydrolysis using water in the presence of acid.

For example, an amine group may be protected, for example, as an amide (—NRCO—R), for example: as an acetamide (—NHCO—CH₃); or as a carbamate (—NRCO—OR), for example: as a benzyloxy carbamate (—NHCO—OCH₂C₆H₅, —NH-Cbz), as a t-butoxy carbamate (—NHCO—OC(CH₃)₃, —NH-Boc); as a 2-biphenyl-2-propoxy carbamate (—NHCO—OC(CH₃)₂C₆H₄C₆H₅, —NH-Bpoc), as a ₉-fluorenylmethoxy carbamate (—NH—Fmoc), as a 6-nitroveratryloxy carbamate (—NH—Nvoc), as a 2-trimethylsilylethyloxy carbamate (—NH—Teoc), a 2,2,2-trichloroethyloxy carbamate (—NH-Troc), as an allyloxy amide (—NH—Alloc), or as a 2 (-phenylsulfonyl)ethyloxy carbamate (—NH—Psec); or, in suitable cases (e.g., cyclic amines), as a nitroxide radical (>N—O·); or, in suitable cases (e.g., heterocyclic nitrogens), as a 2-trimethylsilylethoxymethyl (N-SEM).

For example, a carboxylic acid group may be protected as an ester for example, as: an C₁₋₇alkyl ester (e.g., a methyl ester; a t-butyl ester); a C₁₋₇haloalkyl ester (e.g., a 2,2,2-trihaloethyl ester); a 2-tri (C₁₋₇alkyl) silyl-ethyl ester; or a C₅₋₂₀aryl-C₁₋₇alkyl ester (e.g., a benzyl ester; a nitrobenzyl ester); or as an amide or hydrazide, for example, as acetamide or a N,N,N′-trimethylhydrazide.

For example, a thiol group may be protected as a thioether (—SR), for example, as: a benzyl thioether; an acetamidomethyl ether (—S—CH₂NHC(═O)CH₃).

### Prodrugs

It may be convenient or desirable to prepare, purify, and/or handle the compound in the form of a prodrug. The term “prodrug,” as used herein, pertains to a compound, which yields the desired active compound in vivo. Typically, the prodrug is inactive, or less active than the desired active compound, but may provide advantageous handling, administration, or metabolic properties.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O) OH) in the parent compound, with, where appropriate, prior protection of any other reactive groups present in the parent compound, followed by deprotection if required.

Also, some prodrugs are activated enzymatically to yield the active compound, or a compound, which, upon further chemical reaction, yields the active compound (for example, as in antibody directed enzyme prodrug therapy (ADEPT), gene directed enzyme prodrug therapy (GDEPT), ligand -directed enzyme prodrug therapy (LIDEPT), etc.). For example, the prodrug may be a sugar derivative or other glycoside conjugate, or may be an amino acid ester derivative.

### Compositions

Also described herein is a composition (e.g., a pharmaceutical composition) comprising a BAA compound, as described herein, and a pharmaceutically acceptable carrier, diluent, or excipient.

Also described herein is a method of preparing a composition (e.g., a pharmaceutical composition) comprising mixing a BAA compound, as described herein, and a pharmaceutically acceptable carrier, diluent, or excipient.

### Uses

The BAA compounds, as described herein, inhibit PKMYT1 (e.g., inhibit or reduce or block the activity or function of PKMYT1).

Accordingly, the BAA compounds, as described herein, are useful, for example, in the treatment of disorders (e.g., diseases) that are ameliorated by the inhibition of PKMYT1 (e.g., by the inhibition or reduction or blockage of the activity or function of PKMYT1).

### Use in Methods of Inhibiting PKMYT1

Also described herein is a method of inhibiting PKMYT1 (e.g., inhibiting or reducing or blocking the activity or function of PKMYT1), in vitro or in vivo, comprising contacting the PKMYT1 with an effective amount of a BAA compound, as described herein.

Also described herein is a method of inhibiting PKMYT1 (e.g., inhibiting or reducing or blocking the activity or function of PKMYT1) in a cell, in vitro or in vivo, comprising contacting the cell with an effective amount of a BAA compound, as described herein.

In one embodiment, the method is performed in vitro.

In one embodiment, the method is performed in vivo.

In one embodiment, the BAA compound is provided in the form of a pharmaceutically acceptable composition.

One of ordinary skill in the art is readily able to determine whether or not a candidate compound inhibits PKMYT1 (e.g., inhibits or reduces or blocks the activity or function of PKMYT1). For example, suitable assays are described herein and/or are known in the art.

One of ordinary skill in the art is readily able to determine whether or not a candidate compound inhibits PKMYT1 (e.g., inhibits or reduces or blocks or the activity or function of PKMYT1) in a cell. For example, a sample of cells may be grown in vitro and a compound brought into contact with said cells, and the effect of the compound on those cells observed. As an example of “effect,” the morphological status of the cells (e.g., alive or dead, etc.) may be determined. Where the compound is found to exert an influence on the cells, this may be used as a prognostic or diagnostic marker of the efficacy of the compound in methods of treating a subject (e.g., patient) carrying cells of the same cellular type. As another example of “effect,” the direct interaction of the compound with the target in cells could be measured (e.g., “target engagement assay”) using, e.g., a colorimetric, fluorescent, or luminescent readout.

### Use in Methods of Inhibiting Cell Proliferation, Etc.

The BAA compounds described herein may e.g., (a) regulate (e.g., inhibit) cell proliferation; (b) inhibit cell cycle progression; (c) promote apoptosis; (d) reduce clonogenicity; (e) reduce tumoursphere growth or self-renewal; (f) enhance impact of DNA-damaging agents on cell killing; or (g) a combination of one or more of these.

Accordingly, also described herein is a method of regulating (e.g., inhibiting) cell proliferation (e.g., proliferation of a cell), inhibiting cell cycle progression, promoting apoptosis, reducing clonogenicity, reducing tumoursphere growth or self-renewal, or a combination of one or more these, in vitro or in vivo, comprising contacting a cell with an effective amount of a BAA compound, as described herein.

In one embodiment, the method is performed in vitro.

In one embodiment, the method is performed in vivo.

In one embodiment, the BAA compound is provided in the form of a pharmaceutically acceptable composition.

Any type of cell may be treated or targeted, including for example blood (including, e.g., neutrophils, eosinophils, basophils, lymphocytes, monocytes, erythrocytes, thrombocytes), lung, gastrointestinal (including, e.g., bowel, colon), breast (mammary), ovarian, prostate, liver (hepatic), kidney (renal), bladder, pancreas, brain, and skin cells.

One of ordinary skill in the art is readily able to determine whether or not a candidate compound regulates (e.g., inhibits) cell proliferation, etc. For example, assays which may conveniently be used to assess the activity offered by a particular compound are described herein and/or are known in the art.

The BAA compounds described herein may inhibit cell migration and invasion, e.g., inhibit metastasis.

The BAA compounds described herein may restore sensitivity to another agent in a resistant cell population.

The BAA compounds described herein may prevent emergence of resistance to another agent in a cell population.

The BAA compounds described herein may enhance the impact of other agents on DNA damage and subsequent cell killing. Such agents can be therapeutic compounds generating DNA damage or interfering with DNA damage response.

### Use in Methods of Therapy

Also described herein is a BAA compound, as described herein, for use in a method of treatment of the human or animal body by therapy, for example, for use in a method of treatment of a disorder (e.g., a disease) as described herein.

Also described herein is use of a BAA compound, as described herein, in a method of treatment of the human or animal body by therapy, for example, in a method of treatment of a disorder (e.g., a disease) as described herein.

### Use in the Manufacture of Medicaments

Also described herein is use of a BAA compound, as described herein, in the manufacture of a medicament, for example, for use in a method of treatment, for example, for use in a method of treatment of a disorder (e.g., a disease) as described herein.

In one embodiment, the medicament comprises the BAA compound.

### Methods of Treatment

Also described herein is a method of treatment, for example, a method of treatment of a disorder (e.g., a disease) as described herein, comprising administering to a subject in need of treatment a therapeutically-effective amount of a BAA compound, as described herein, preferably in the form of a pharmaceutical composition.

### Disorders Treated-Disorders Ameliorated by the Inhibition of PKMYT1

In one embodiment (e.g., of compounds for use in methods of therapy, of use in methods of therapy, of use in the manufacture of medicaments, of methods of treatment), the treatment is treatment of a disorder (e.g., a disease) that is ameliorated by the inhibition of PKMYT1 (e.g., by the inhibition or reduction or blockage of the activity or function of PKMYT1).

### Disorders Treated

In one embodiment (e.g., of compounds for use in methods of therapy, of use in methods of therapy, of use in the manufacture of medicaments, of methods of treatment), the treatment is treatment of a disorder (e.g., a disease), for example, a proliferative disorder, cancer, etc., as described herein.

### Proliferative Disorders

In one embodiment, the disorder is: a proliferative disorder.

The term “proliferative disorder,” as used herein, pertains to an unwanted or uncontrolled cellular proliferation of excessive or abnormal cells which is undesired, such as neoplastic or hyperplastic growth.

In one embodiment, the proliferative disorder is characterised by benign, pre-malignant, malignant, pre-metastatic, metastatic, or non-metastatic cellular proliferation, including for example: neoplasms, hyperplasias, tumours (e.g., histocytoma, glioma, astrocyoma, osteoma), cancers, psoriasis, bone diseases, fibroproliferative disorders (e.g., of connective tissues), pulmonary fibrosis, atherosclerosis, and smooth muscle cell proliferation in the blood vessels, such as stenosis or restenosis following angioplasty.

### Disorders Treated—Proliferative Disorders

In one embodiment (e.g., for use in methods of therapy, of use in the manufacture of medicaments, of methods of treatment), the treatment is treatment of a proliferative disorder.

The term “proliferative disorder,” as used herein, pertains to an unwanted or uncontrolled cellular proliferation of excessive or abnormal cells which is undesired, such as neoplastic or hyperplastic growth.

In one embodiment, the treatment is treatment of: a proliferative disorder characterised by benign, pre-malignant, or malignant cellular proliferation.

In one embodiment, the treatment is treatment of a proliferative disorder characterised by, or further characterised by: inappropriate activity and/or expression of PKMYT1, CCNE1, FBXW7, or PPP2A or one of its subunits.

In one embodiment, the treatment is treatment of a proliferative disorder characterised by, or further characterised by: overexpression of PKMYT1 or CCNE1.

In one embodiment, the treatment is treatment of a proliferative disorder characterised by, or further characterised by overexpression of PKMYT1.

In one embodiment, the treatment is treatment of a proliferative disorder characterised by, or further characterised by overexpression of CCNE1.

In one embodiment, the treatment is treatment of a proliferative disorder characterised by, or further characterised by: inactivation, decreased activity, or decreased expression of FBXW7 or PPP2R2A.

In one embodiment, the treatment is treatment of a proliferative disorder characterised by, or further characterised by inactivation of FBXW7.

In one embodiment, the treatment is treatment of a proliferative disorder characterised by, or further characterised by decreased activity or decreased expression of PPP2R2A.

In one embodiment, the treatment is treatment of cancer.

### Disorders Treated—Cancer

In one embodiment (e.g., of use in methods of therapy, of use in the manufacture of medicaments, of methods of treatment), the treatment is treatment of cancer.

Included among cancers are:
(1) Carcinomas, including tumours derived from stratified squamous epithelia (squamous cell carcinomas) and tumours arising within organs or glands (adenocarcinomas). Examples include breast, colon, lung, prostate, ovary.
(2) Sarcomas, including: osteosarcoma and osteogenic sarcoma (bone); chondrosarcoma (cartilage); leiomyosarcoma (smooth muscle); rhabdomyosarcoma (skeletal muscle); mesothelial sarcoma and mesothelioma (membranous lining of body cavities); fibrosarcoma (fibrous tissue); angiosarcoma and haemangioendothelioma (blood vessels); liposarcoma (adipose tissue); glioma and astrocytoma (neurogenic connective tissue found in the brain); myxosarcoma (primitive embryonic connective tissue); mesenchymous and mixed mesodermal tumour (mixed connective tissue types).
(3) Myeloma.
(4) Haematopoietic tumours, including: myelogenous and granulocytic leukaemia (malignancy of the myeloid and granulocytic white blood cell series), e.g., chronic myeloid leukemia (CML), acute myeloid leukemia (AML); lymphatic, lymphocytic, and lymphoblastic leukaemia (malignancy of the lymphoid and lymphocytic blood cell series), e.g., acute lymphoblastic leukemia (ALL), chronic lymphocytic leukemia (CLL); polycythaemia vera (malignancy of various blood cell products, but with red cells predominating).
(5) Lymphomas, including: Hodgkin and Non-Hodgkin lymphomas.
(6) Mixed Types, including, e.g., adenosquamous carcinoma; mixed mesodermal tumour; carcinosarcoma; teratocarcinoma.

In one embodiment, the cancer is:
a bone or muscle sarcoma, for example: bone cancer; bone sarcoma; chondrosarcoma; Ewing's sarcoma; heart cancer; leiomyosarcoma; malignant fibrous histiocytoma of bone; osteosarcoma; or rhabdomyosarcoma;
a brain and nervous system cancer, for example: astrocytoma; brain cancer; brainstem glioma; cerebellar astrocytoma; cerebral astrocytoma; ependymoma; glioblastoma; glioma; medulloblastoma; neuroblastoma; oligodendroglioma; pilocytic astrocytoma; pineal astrocytoma; pituitary adenoma; primitive neuroectodermal tumor; schwannoma; or visual pathway and hypothalamic glioma;
a breast cancer, for example: breast cancer; invasive cribriform carcinoma; inflammatory breast cancer; invasive lobular carcinoma; medullary carcinoma; male breast cancer; phyllodes tumor; or tubular carcinoma;
an endocrine system cancer, for example: adrenal gland cancer; adrenocortical carcinoma; papillary thyroid cancer; follicular thyroid cancer; islet cell carcinoma; multiple endocrine neoplasia syndrome; parathyroid cancer; pheochromocytoma; thyroid cancer; or thyroid gland cancer;
an eye cancer, for example: retinoblastoma; or uveal melanoma;
a gastrointestinal cancer, for example:
   anal cancer; appendix cancer; biliary tract cancer; bowel cancer; cholangiocarcinoma; colon adenocarcinoma; colon adenoma; colon cancer; exocrine pancreatic carcinoma; extrahepatic bile duct cancer; gallbladder cancer; gastric (stomach) cancer; gastrointestinal cancer; gastrointestinal carcinoid tumor; gastrointestinal carcinoid tumor; gastrointestinal stromal tumor (GIST); hepatocellular cancer; hepatoblastoma; kidney cancer; large bowel cancer; liver cancer; ocolorectal cancer; pancreatic cancer; rectal cancer; or small bowel cancer;
a genitourinary or gynecologic cancer, for example: bladder cancer; cervical cancer; endometrial cancer; extragonadal germ cell tumor; genito-urinary cancer; gestational trophoblastic tumor; gynaecological cancer; ovarian cancer; ovarian epithelial cancer; ovarian germ cell tumor; penile cancer; prostate cancer; renal cell carcinoma; renal pelvis and ureter, transitional cell cancer; seminoma; teratocarcinoma; testicular cancer; transitional cell cancer of the ureter and renal pelvis; urethral cancer; uterine sarcoma; vaginal cancer; vulvar cancer; or Wilms tumor;
a cancer of the head or neck, for example: esophageal cancer; head and neck cancer; head and neck squamous cell carcinoma; hypopharyngeal cancer; nasopharyngeal cancer; nasopharyngeal carcinoma; oral cancer; oropharyngeal cancer; paranasal sinus and nasal cavity cancer; pharyngeal cancer; or salivary gland cancer;
a hematopoietic cancer, for example:
a plasma cell neoplasm, for example, plasmacytoma or multiple myeloma;
a leukemia, for example: acute biphenotypic leukemia; acute eosinophilic leukemia; acute lymphoblastic leukemia; acute myeloid dendritic cell leukemia; acute myeloid leukemia; acute promyelocytic leukemia; B-cell prolymphocytic leukemia; chronic lymphocytic leukemia; chronic myelogenous leukemia; hairy cell leukemia; large granular lymphocytic leukemia; mast cell leukemia; precursor B lymphoblastic leukemia; T-cell prolymphocytic leukemia;
a lymphoma, for example: AIDS-related lymphoma; anaplastic large cell lymphoma; angioimmunoblastic T-cell lymphoma; Burkitt's lymphoma; cutaneous T-cell lymphoma; diffuse large B-cell lymphoma; follicular lymphoma; hepatosplenic T-cell lymphoma; Hodgkin's lymphoma; intravascular large B-cell lymphoma; lymphomatoid granulomatosis; lymphoplasmacytic lymphoma; mantle cell lymphoma; marginal zone B-cell lymphoma; mediastinal large B cell lymphoma; mucosa-associated lymphoid tissue lymphoma; mycosis fungoides; nodal marginal zone B cell lymphoma; non-Hodgkin lymphoma; plasmablastic lymphoma; primary central nervous system lymphoma; primary cutaneous follicular lymphoma; primary cutaneous immunocytoma; primary effusion lymphoma; Sézary syndrome; or splenic marginal zone lymphoma; or a myelodysplastic syndrome;
a skin cancer, for example: basal cell carcinoma; dermatofibrosarcoma protuberans; fibrosarcoma; keratoacanthoma; malignant melanoma; melanoma; Merkel cell carcinoma; sebaceous carcinoma; or squamous cell carcinoma;
a thoracic and respiratory cancer, for example: adenocarcinoma; bronchial adenoma; bronchial carcinoid; laryngeal cancer; lung cancer; mediastinum cancer; mesothelioma; non-small cell lung cancer; peritoneal cancer; pleuropulmonary blastoma; small cell lung cancer; thymic carcinoma; or thymoma carcinoma;
an HIV/AIDS related cancer, for example, Kaposi sarcoma;
or other cancer, for example, epithelioid hemangioendothelioma; desmoplastic small round cell tumor; or liposarcoma.

In one embodiment, the cancer is:
endometrial cancer, uterine cancer, ovarian cancer, breast cancer, gastric cancer, bladder cancer, pancreatic cancer, mesothelioma, kidney cancer, stomach cancer, esophageal cancer, colorectal cancer, glioblastoma, lung cancer, or lung squamous cell carcinoma.

In one embodiment, the cancer is endometrial cancer.

In one embodiment, the cancer is uterine cancer.

In one embodiment, the cancer is ovarian cancer.

In one embodiment, the cancer is breast cancer.

In one embodiment, the cancer is gastric cancer.

In one embodiment, the cancer is bladder cancer.

In one embodiment, the cancer is pancreatic cancer.

In one embodiment, the cancer is mesothelioma.

In one embodiment, the cancer is kidney cancer.

In one embodiment, the cancer is stomach cancer.

In one embodiment, the cancer is esophageal cancer.

In one embodiment, the cancer is colorectal cancer.

In one embodiment, the cancer is glioblastoma.

In one embodiment, the cancer is lung cancer.

In one embodiment, the cancer is lung squamous cell carcinoma.

In one embodiment, the cancer is characterised by, or further characterised by inappropriate activity (e.g., overexpression) of PKMYT1. For example, in one embodiment, the cancer is:
lung squamous cell carcinoma, lung adenocarcinoma, uterine corpus endometrial carcinoma, breast cancer, breast invasive carcinoma, hepatocellular carcinoma, clear-cell renal-cell carcinoma, kidney chromophobe cancer, renal papillary cell carcinoma, head and neck squamous cell carcinoma, colon adenocarcinoma, stomach adenocarcinoma, thyroid carcinoma, prostate adenocarcinoma, adrenocortical carcinoma, lower grade glioma, mesothelioma, pancreatic adenocarcinoma, skin cutaneous melanoma, uveal melanoma.

In one embodiment, the cancer is characterised by, or further characterised by involvement of PKMYT1 in progression, invasion and/or metastasis. For example, in one embodiment, the cancer is:
non-small cell lung cancer, osteosarcoma, clear cell renal cell carcinoma, oral squamous cell carcinoma, gastric cancer, prostate cancer, oesophageal squamous cell carcinoma, colorectal cancer, hepatocellular carcinoma, ovarian cancer, neuroblastoma (in particular, with MYCN amplification), glioblastoma, acute lymphoblastic leukemia, multiple myeloma, Kaposi's sarcoma, primary effusion lymphoma (PEL), or the plasmablastic variant of multicentric Castleman's disease.

In one embodiment, the cancer is characterised by, or further characterised by inappropriate activity (e.g., overexpression) of CCNE1. For example, in one embodiment, the cancer is:
uterine carcinosarcoma, uterine cancer, endometrial cancer, breast cancer, ovarian cancer, stomach cancer, colorectal cancer, bladder cancer, oesophageal cancer, cervical cancer, sarcoma, lung squamous cancer, adenoid cystic carcinoma, pancreatic cancer, mesothelioma, lung adenocarcinoma, head & neck cancer, diffuse large B-cells lymphoma, or liver cancer.

In one embodiment, the cancer is characterised by, or further characterised by inappropriate activity (e.g., overexpression) of CCNE1. For example, in one embodiment, the cancer is:
uterine carcinosarcoma, uterine cancer, endometrial cancer, breast cancer, ovarian cancer, stomach cancer, colorectal cancer, bladder cancer, oesophageal cancer, cervical cancer, sarcoma, or lung squamous cancer.

In one embodiment, the cancer is uterine carcinosarcoma (UCS) or uterine serous carcinoma (USC).

In one embodiment, the cancer is high-grade serous ovarian carcinoma (HGSOC).

In one embodiment, the cancer is high-grade serous ovarian cancer with CCNE1 amplification.

In one embodiment, the cancer is triple-negative breast cancer (TNBC).

In one embodiment, the cancer is characterised by, or further characterised by inappropriate activity (e.g., inactivation) of FBXW7. For example, in one embodiment, the cancer is:
uterine carcinosarcoma, uterine cancer, endometrial cancer, breast cancer, ovarian cancer, stomach cancer, colorectal cancer, bladder cancer, oesophageal cancer, cervical cancer, sarcoma, lung squamous cancer, or head & neck cancer.

In one embodiment, the cancer is characterised by, or further characterised by inappropriate activity (e.g., inactivation) of FBXW7. For example, in one embodiment, the cancer is:
uterine carcinosarcoma, endometrial cancer, colorectal cancer, cervical cancer, bladder cancer, head & neck cancer, gastric cancer, or lung squamous cells carcinoma.

In one embodiment, the cancer is uterine carcinosarcoma (UCS) or uterine serous carcinoma (USC).

In one embodiment, the cancer is characterised by, or further characterised by inappropriate activity (e.g., inactivation, decreased activity, decreased expression) of PPP2R2A. For example, in one embodiment, the cancer is:
prostate adenocarcinoma, ovarian serous cystadenocarcinoma, rectum adenocarcinoma, bladder urothelial carcinoma, colorectal adenocarcinoma, breast invasive carcinoma, uterine corpus endometrial carcinoma, uterine carcinosarcoma, liver hepatocellular carcinoma, lung squamous cell carcinoma, or lung adenocarcinoma.

In one embodiment, the cancer (e.g., as above) is characterised, or further characterised, as treatment resistant cancer, e.g., chemotherapy-resistant cancer, radiotherapy-resistant cancer, and/or immunotherapy-resistant cancer.

In one embodiment, the treatment resistant cancer is resistant to standard of care therapy.

In one embodiment, the treatment resistant cancer is resistant to one or more of PARP inhibitors, cisplatin, WEE1 inhibitors and Cdk4/6 inhibitors.

In one embodiment, the treatment resistant cancer is recombination proficient ovarian cancer.

In one embodiment, the treatment resistant cancer is HER2-ER+breast cancer with Cdk4/6 resistance.

In one embodiment, the cancer (e.g., as above) is characterised, or further characterised, as metastatic cancer.

The anti-cancer effect may arise through one or more mechanisms, including but not limited to, the regulation of cell proliferation, the inhibition of cell cycle progression, the inhibition of angiogenesis (the formation of new blood vessels), the inhibition of metastasis (the spread of a tumour from its origin), the inhibition of cell migration (the spread of cancer cells to other parts of the body), the inhibition of invasion (the spread of tumour cells into neighbouring normal structures), the promotion of apoptosis (programmed cell death), death by necrosis, or induction of death by autophagy. The compounds described herein may be used in the treatment of the cancers described herein, independent of the mechanisms discussed herein.

### Treatment

The term “treatment,” as used herein in the context of treating a disorder (e.g., disease), pertains generally to treatment of a human or an animal (e.g., in veterinary applications), in which some desired therapeutic effect is achieved, for example, the inhibition of the progress of the disorder (including, e.g., a reduction in the rate of progress, a halt in the rate of progress), alleviation of symptoms of the disorder, amelioration of the disorder, and cure of the disorder. Treatment as a prophylactic measure (i.e., prophylaxis) is also included. For example, use with subjects (e.g., patients) who have not yet developed the disorder, but who are at risk of developing the disorder, is encompassed by the term “treatment.”

For example, treatment of cancer includes reducing the progress of cancer, alleviating the symptoms of cancer, reducing the incidence of cancer, prophylaxis of cancer, etc.

The term “therapeutically-effective amount,” as used herein, pertains to that amount of a compound, or a material, composition, or dosage form comprising a compound, which is effective for producing some desired therapeutic effect, commensurate with a reasonable benefit/risk ratio, when administered in accordance with a desired treatment regimen.

### Combination Therapies

The term “treatment” as used herein includes combination treatments and therapies, in which two or more treatments or therapies are combined, for example, sequentially or simultaneously. For example, the BAA compounds described herein may also be used in combination therapies, e.g., in conjunction with other agents.

Accordingly, also described herein is a BAA compound, as described herein, in combination with one or more (e.g., 1, 2, 3, 4, etc.) additional therapeutic agents.

For example, also described herein is a BAA compound, as described herein, for use in a method of treatment of the human or animal body by therapy, for example, for use in a method of treatment of a disorder (e.g., a disease) as described herein, where the BAA compound is administered in combination with one or more (e.g., 1, 2, 3, 4, etc.) additional therapeutic agents.

Also described herein is use of a BAA compound, as described herein, in a method of treatment of the human or animal body by therapy, for example, in a method of treatment of a disorder (e.g., a disease) as described herein, where the BAA compound is administered in combination with one or more (e.g., 1, 2, 3, 4, etc.) additional therapeutic agents.

Also described herein is use of a BAA compound, as described herein, in a method of treatment of the human or animal body by therapy, for example, in a method of treatment of a disorder (e.g., a disease) as described herein, where the BAA compound is administered in combination with one or more (e.g., 1, 2, 3, 4, etc.) additional therapeutic agents.

Also described herein is use of a BAA compound, as described herein, in the manufacture of a medicament, for example, for use in a method of treatment, for example, for use in a method of treatment of a disorder (e.g., a disease) as described herein, where the BAA compound is administered in combination with one or more (e.g., 1, 2, 3, 4, etc.) additional therapeutic agents.

In one embodiment, the medicament comprises the BAA compound. Also described herein is a method of treatment, for example, a method of treatment of a disorder (e.g., a disease) as described herein, comprising administering to a subject in need of treatment a therapeutically effective amount of a BAA compound, as described herein, optionally in the form of a pharmaceutical composition, where the BAA compound is administered in combination with one or more (e.g., 1, 2, 3, 4, etc.) additional therapeutic agents.

The particular combination would be at the discretion of the physician who would select dosages using their common general knowledge and dosing regimens known to a skilled practitioner.

The agents (e.g., the BAA compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (e.g., the BAA compound described here, plus one or more other agents) may be formulated together in a single dosage form, or alternatively, the individual agents may be formulated separately, and optionally may be presented together in the form of a kit, optionally with instructions for their use.

In one embodiment, the other agent (e.g., the additional therapeutic agent) is an immunotherapeutic agent, for example, an immune checkpoint inhibitor.

In one embodiment, the other agent (e.g., the additional therapeutic agent, for example the additional anti-cancer agent) is an immunotherapy agent, such as a monoclonal antibody (for example trastuzumab, bevacizumab, cetuximab, daratumumab, or naxitamab, necitumumab, obinutuzumab, ofatumumab, panitumumab, pertuzumab, ramucirumab, or rituximab), a bispecific antibody (for example blinatumomab), an immune checkpoint inhibitor (for example ipilimumab, nivolumab, pembrolizumab, cemiplimab, atezolizumab, durvalumab, avelumab, dostarlimab, or tremelimumab), an immunomodulator (for example imiquimod, thalidomide, lenalidomide, or ponalidomide), a cytokine such as an interleukin (for example IL-2 aldesleukin), an interferon (for example IFNa), an oncolytic virus (for example talimogene laherparepvec), or a T-cell therapy.

In one embodiment, the other agent is an antibody-drug conjugate (i.e. an “ADC”, for example brentuximab vedotin, inotuzumab ozogamicin, mirvetuximab soravtansine-gynx, fam-trastuzumab deruxtecan-nxki, ado-trastuzumab emtansine, gemtuzumab ozogamicin, enfortumab vedotin-ejfv, polatuzumab vedotin-piiq, tisotumab vedotin-tftv, sacituzumab govitecan-hziy, loncastuximab tesirine-lpyl, or distamab vedotin).

In one embodiment, the other agent is a DNA-damaging agent, such as an alkylating agent (for example cis-platin, oxaliplatin, carboplatin, cyclophosphamide, melphalan, chlorambucil, bendamustine, temozolomide, trabectidin, mitomycin C, or dacarbazine); an antimetabolite (for example capecitabine, gemcitabine, 5-fluorouracil, fluoropyrimidine, trifluridine and tipiracil, cytarabine, or methotrexate); a DNA intercalator (for example an anthracycline like doxorubicin, epirubicin, or daunorubicin), an antibiotic (for example bleomycin, dactinomycin, or mithramycin); a topoisomerase 1 inhibitor (for example a camptothecin such as irinotecan, or topotecan), a topoisomerase 2 inhibitor (for example etoposide), a microtubule-targeting agent (for example a taxane such as paclitaxel; or a vinca alkaloid such as vincristine, vinblastine, vindesine, vinorelbine, or eribulin); or an antibiotic (for example bleomycin, or mitomycin-C).

In one embodiment, the other agent is a topoisomerase I inhibitor (for example irinotecan or topotecan), a topoisomerase II inhibitor (for example etoposide), or an antimetabolite (for example gemcitabine).

In one embodiment, the other agent is an alkylating agent (for example cis-platin).

In one embodiment, the other agent is a topoisomerase I inhibitor (for example irinotecan or topotecan).

In one embodiment, the other agent is a topoisomerase II inhibitor (for example etoposide).

In one embodiment, the other agent is an antimetabolite (for example gemcitabine).

In one embodiment, the other agent is a DNA-damage repair inhibitor (for example a PARP inhibitor such as olaparib, rucaparib, niraparib, or talazoparib; or a PARG inhibitor; or a USP1 inhibitor).

In one embodiment, the other agent is double strand -break repair inhibitor (for example a Pole inhibitor, or a RAD51 inhibitor).

In one embodiment, the other agent is a signalling pathway inhibitor, such as a kinase inhibitor (for example abemaciclib, acalabrutinib, afatinib, alectinib, avapritinib, axitinib, baricitinib, belumosudil, binimetinib, bosutinib, brigatinib, cabozantinib, capmatinib, ceritinib, cobimetinib, rizotinib, dabrafenib, dacomitinib, dasatinib, encorafenib, entrectinib, erdafitinib, erlotinib, everolimus, fedratinib, fostamatinib, gefitinib, gilteritinib, ibrutinib, imatinib, infigratinib, lapatinib, larotrectinib, lenvatinib, lorlatinib, midostaurin, mobocertinib, neratinib, netarsudil, nilotinib, nintedanib, osimertinib, palbociclib, pazopanib, pemigatinib, pexidartinib, ponatinib, pralsetinib, regorafenib, ribociclib, ripretinib, ruxolitinib, selpercatinib, selumetinib, sirolimus, sorafenib, sunitinib, temsirolimus, tepotinib, tivozanib, tofacitinib, trametinib, trilaciclib, tucatinib, upadacitinib, vandetanib, vemurafenib, or zanubrutinib).

In one embodiment, the other agent is a cell cycle targeting inhibitor, such as a CDK4/6 inhibitor (for example palbociclib, abemaciclib, or ribociclib).

In one embodiment, the other agent is an agent targeting DNA damage checkpoints, such as an ATR inhibitor (for example ceralasertib, gartisertib, tuvusertib, elimusertib, or camonsertib), an ATM inhibitor (for example AZD0156), a CHK1 inhibitor (for example prexasertib), a CHK2 inhibitor, a WEE1 inhibitor (for example adavosertib, or azenosertib), a PLK1 inhibitor (for example onvansertib), or an AUR-A inhibitor (for example JAB-2485).

In one embodiment, the other agent is an ATR inhibitor (for example ceralasertib, gartisertib, tuvusertib, elimusertib, or camonsertib), a CHK1 inhibitor (for example prexasertib), or a WEE1 inhibitor (for example adavosertib, or azenosertib).

In one embodiment, the other agent is an ATR inhibitor (for example ceralasertib, gartisertib, tuvusertib or camonsertib), or a WEE1 inhibitor (for example azenosertib).

In one embodiment, the other agent is a CHK1 inhibitor (for example prexasertib).

In one embodiment, the other agent is an ATR inhibitor (for example ceralasertib, gartisertib, tuvusertib, elimusertib, or camonsertib).

In one embodiment, the other agent is a WEE1 inhibitor (for example adavosertib, or azenosertib).

In one embodiment, the agent is a hormone therapy agent, such as an antiestrogen (for example tamoxifen, fulvestrant, toremifene, raloxifene, droloxifene, or idoxifene), an antiandrogen (for example abiraterone, bicalutamide, enzalutamide, flutamide, nilutamide, or cyproterone acetate), an LHRH antagonist or LHRH agonist (for example goserelin, leuprorelin, or buserelin), a progestogen (for example megestrol acetate), an aromatase inhibitor (for example as anastrozole, letrozole, vorazole, or exemestane) an inhibitor of 5α-reductase (for example finasteride) or an analogue of somatostatin (for example lanreotide).

In one embodiment, the other agent is a proteasome inhibitor (for example bortezomib), a histone deacetylase inhibitor (for example vorinostat, romidepsin, panobinostat, or belinostat), or a DNA demethylating agent (for example azacitidine, or decitabine).

In one embodiment, the other agent is radiotherapy, such as radiotherapy comprising treatment with a radiotherapeutic drug (for example lutetium Lu 177 dotatate, lutetium Lu 177 vipivotide tetraxetan, samarium Sm 153 lexidronam, radium Ra 223 dichloride, or Y-90 ibritumomab tiuxetan).

### Other Uses

The BAA compounds described herein may also be used as cell culture additives to inhibit PKMYT1 (e.g., to inhibit or reduce or block the sactivity or function of PKMYT1).

The BAA compounds described herein may also be used as part of an in vitro assay, for example, in order to determine whether a candidate host is likely to benefit from treatment with the compound in question.

The BAA compounds described herein may also be used as a standard, for example, in an assay, in order to identify other active compounds, other PKMYT1 inhibitors, etc.

### Kits

Also describes herein is a kit comprising (a) a BAA compound, as described herein, preferably provided as a composition (e.g., a pharmaceutical composition) and in a suitable container and/or with suitable packaging; and (b) instructions for use, for example, in a method of treatment of a disorder (e.g., a disease) as described herein, for example, written instructions on how to administer the compound.

The written instructions may also include a list of indications for which the BAA compound is a suitable treatment.

### Routes of Administration

The BAA compound or pharmaceutical composition comprising the BAA compound may be administered to a subject by any convenient route of administration, whether systemically/peripherally or topically (i.e., at the site of desired action).

Routes of administration include, for example: oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject

The subject (e.g., patient) may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutan, gibbon), or a human.

Furthermore, the subject (e.g., patient) may be any of its forms of development, for example, a foetus.

In one preferred embodiment, the subject (e.g., patient) is a human.

### Formulations

While it is possible for a BAA compound to be administered alone, it is preferable to present it as a pharmaceutical formulation (e.g., composition, preparation, medicament) comprising at least one BAA compound, as described herein, together with one or more other pharmaceutically acceptable ingredients well known to those skilled in the art, including, for example, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, also described herein are pharmaceutical compositions, as defined above, and methods of making a pharmaceutical composition comprising mixing at least one BAA compound, as described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington: The Science and Practice of Pharmacy, 21st edition, Lippinott Williams and Wilkins, 2005; Remington: The Science and Practice of Pharmacy, 22nd edition, Pharmaceutical Press, 2012; and Handbook of Pharmaceutical Excipients, 7th edition, Pharmaceutical Press, 2012.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The compound may be dissolved in, suspended in, or mixed with one or more other pharmaceutically acceptable ingredients. The compound may be presented in a liposome or other microparticulate which is designed to target the compound, for example, to blood components or one or more organs.

Formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, losenges, pastilles, as well as patches, adhesive plasters, depots, and reservoirs. Losenges typically comprise the compound in a flavoured basis, usually sucrose and acacia or tragacanth. Pastilles typically comprise the compound in an inert matrix, such as gelatin and glycerin, or sucrose and acacia. Mouthwashes typically comprise the compound in a suitable liquid carrier.

Formulations suitable for sublingual administration include tablets, losenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, losenges, pastilles, as well as patches, adhesive plasters, depots, and reservoirs.

Formulations suitable for non- oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent. The tablets may optionally be coated or scored and may be formulated so as to provide slow or controlled release of the compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the compound and a paraffinic or a water-miscible ointment base.

Creams are typically prepared from the compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the compound through the skin or other affected areas. Examples of such dermal penetration enhancers include dimethylsulfoxide and related analogues.

Emulsions are typically prepared from the compound and an oily phase, which may optionally comprise merely an emulsifier (otherwise known as an emulgent), or it may comprise a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichorotetrafluoroethane, carbon dioxide, or other suitable gases.

Formulations suitable for ocular administration include eye drops wherein the compound is dissolved or suspended in a suitable carrier, especially an aqueous solvent for the compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additionally contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/ml to about 10 μg/mL, for example from about 10 ng/ml to about 1 μg/mL. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the BAA compounds, and compositions comprising the BAA compounds, can vary from subject to subject (e.g., from patient to patient). Determining the optimal dosage will generally involve balancing the level of therapeutic benefit against any risk or deleterious side effects. The selected dosage level will depend on a variety of factors including, for example: the activity of the particular BAA compound; the route of administration; the time of administration; the rate of excretion of the BAA compound; the duration of the treatment; other drugs, compounds, and/or materials used in combination; the severity of the disorder; and the species, sex, age, weight, condition, general health, and prior medical history of the subject (e.g., patient). The amount of BAA compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the BAA compound is in the range of about 0.01 mg to about 5000 mg (more typically about 0.1 mg to about 1000 mg, e.g., about 0.1 mg to about 300 mg) per day.

Where the compound is a salt, a solvate, an ester, an amide, a prodrug, or the like, the amount administered is calculated on the basis of the parent compound and so the actual weight to be used is increased proportionately.

### Examples

### Chemical Synthesis

### Abbreviations

{1H} Proton decoupling
aq. Aqueous
BINAP 2,2′-Bis(diphenylphosphino)-1,1′-binaphthyl
DCM Dichloromethane (methylene chloride)
DIAD diisopropyl azodicarboxylate
DIPEA N,N-diisopropylethylamine
DMSO Dimethylsulfoxide
ES Electrospray ionisation
Et₂O Ether (diethyl ether)
EtOAc Ethyl acetate
EtOH Ethanol (ethyl alcohol)
HATU 2-(7-aza-1H-benzotriazole-1-yl)-1,1,3,3-tetramethyluronium hexafluorophosphate
MeCN Acetonitrile
MeOH Methanol (methyl alcohol)
Pd/C Palladium on carbon
Pd₂dba₃ tris(dibenzylideneacetone) dipalladium (0)
—RT Room temperature
Sat. aq. Saturated aqueous
STAB Sodium triacetoxyborohydride
T3P 1-Propanephosphonic anhydride
TFA Trifluroacetic acid
THF Tetrahydrofuran
UPLC Ultra Performance Liquid Chromatography
XantPhos 4,5-bis(diphenylphospheno)-9,9-dimethylxanthene

### Methods

### General Experimental

Flash chromatography was performed using pre-packed silica gel cartridges (RediSep Rf, Isco). Thin layer chromatography was conducted with 5×10 cm plates coated with Merck Type 60 F254 silica gel to a thickness of 0.25 mm. All reagents obtained from commercial sources were used without further purification. Anhydrous solvents were obtained from the Sigma-Aldrich Chemical Company Ltd. or Fisher Chemicals Ltd., and used without further drying. HPLC grade solvents were obtained from Fisher Chemicals Ltd.

All compounds were >90% purity as determined by examination of both the LCMS and 1H NMR spectra unless otherwise indicated. Where CI or Br were present, expected isotopic distribution patterns were observed.

### NMR

Proton (¹H) and carbon (¹³C) and (¹⁹F) NMR spectra were recorded on a 300 MHz Bruker or 400 MHz Jeol spectrometer. Solutions were typically prepared in either deuterated chloroform (Chloroform-d), deuterated methanol (Methanol-d₄) or deuterated dimethylsulfoxide (DMSO-d₆) with chemical shifts referenced to tetramethylsilane (TMS) or deuterated solvent as an internal standard. ¹H NMR data are reported indicating the chemical shift (δ), the integration (e.g., ¹H), the multiplicity (s, singlet; d, doublet; t, triplet; q, quartet; m, multiplet; br, broad; dd, doublet of doublets) and the coupling constant (J) in Hz. Deuterated solvents were purchased from the Sigma-Aldrich Chemical Company, Goss or Fluorochem.

### Analytical LCMS

LCMS analyses were performed on a Waters Acquity UPLC using BEH C₁₈ 1.7 μM columns
(2.1×50 mm) with a diode array detector coupled to a SQD mass spectrometer with optional ELS detection (Acquity UPLC ELS Detector) or, a Waters Acquity I-Class UPLC using BEH C₁₈ 1.7 μM columns (2.1×50 mm) with a diode array detector coupled to a QDa mass spectrometer. Analyses were performed with either buffered acidic or basic solvents using gradients as detailed below:
Low pH:
Solvent A—Water+10 mM ammonium hydrogen carbonate+0.1% formic acid
Solvent B—MeCN+5% water+0.1% formic acid
High pH:
Solvent A—Water+10 mM ammonium hydrogen carbonate+0.1% ammonia solution
Solvent B—MeCN+5% water+0.1% ammonia solution
Gradient:

| | Flow rate | | |
|---|---|---|---|
| Time | (mL/min) | % Solvent A | % Solvent B |
| 0 | 0.6 | 95 | 5 |
| 1.2 | 0.6 | 5 | 95 |
| 1.7 | 0.6 | 5 | 95 |
| 1.8 | 0.6 | 95 | 5 |

For some compounds, the following gradients were used:

### Acidic 2 min

0.1% v/v Formic acid in 10 mM ammonium formate [Eluent A]; 0.1% v/v Formic acid in MeCN [Eluent B]; flow rate 0.8 mL/min; column oven 50° C.; sample manager 20° C.; injection volume 2 μL and 1.5 minutes equilibration time between samples, on a Waters Acquity UPLC BEH C18 column (2.1×50 mm, 1.7 μm).

### Gradient:

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

### Acidic 4 min

0.1% v/v formic acid in 10 mM ammonium formate [Eluent A]; 0.1% v/v formic acid in MeCN [Eluent B]; flow rate 0.8 mL/min; column oven 50° C.; sample manager 20° C.; injection volume 2 μL and 1.5 minutes equilibration time between samples on a Waters Acquity UPLC BEH C18 column
(2.1×50 mm, 1.7 μm).

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

### Acidic 6 min (“Acidic Prep Analysis”)

0.1% v/v formic acid in 10 mM ammonium formate [Eluent A]; 0.1% v/v formic acid in MeCN [Eluent B]; flow rate 0.8 mL/min; column oven 50° C.; sample manager 20° C.; injection volume 2 μL and 1.5 minutes equilibration time between samples on a Waters Acquity UPLC BEH C18 column
(2.1× 50 mm, 1.7 μm).

| Time (min) | Eluent A (%) | Eluent B (%) |
|---|---|---|
| 0.00 | 95 | 5 |
| 0.30 | 95 | 5 |
| 6.00 | 5 | 95 |
| 6.10 | 95 | 5 |
| 7.00 | 95 | 5 |
| 2.00 | 95 | 5 |
| 4.00 | 95 | 5 |
| 7.00 | 95 | 5 |

### Basic 2 min

0.1% ammonia in water [Eluent A]; 0.1% ammonia in MeCN [Eluent B]; flow rate 0.8 mL/min; column oven 50° C.; sample manager 20° C.; injection volume 2 μL and 1.5 minutes equilibration time between samples on a Waters Acquity UPLC BEH C18 column (2.1×50 mm, 1.7 μm).

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

### Basic 4 min

0.1% ammonia in water [Eluent A]; 0.1% ammonia in MeCN [Eluent B]; flow rate 0.8 mL/min; column oven 50° C.; sample manager 20° C.; injection volume 2 μL and 1.5 minutes equilibration time between samples on a Waters Acquity UPLC BEH C18 column (2.1×50 mm, 1.7 μm).

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

### Basic 6 min (“Basic Prep Analysis”)

0.1% ammonia in water [Eluent A]; 0.1% ammonia in MeCN [Eluent B]; flow rate 0.8 mL/min; column oven 50° C.; sample manager 20° C.; injection volume 2 μL and 1.5 minutes equilibration time between samples on a Waters Acquity UPLC BEH C18 column (2.1×50 mm, 1.7 μm).

| Time (min) | Eluent A (%) | Eluent B (%) |
|---|---|---|
| 0.00 | 95 | 5 |
| 0.30 | 95 | 5 |
| 6.00 | 5 | 95 |
| 6.10 | 95 | 5 |
| 7.00 | 95 | 5 |

### Preparative HPLC-MS

Some compounds were purified by preparative HPLC on a Waters FractionLynx MS autopurification system, with a Phenomonex Gemini NX 5 μm C18, 100 mm×21.2 mm i.d. column (for low pH runs) or a Waters XBridge 5 μm C18, 100 mm×19 mm i.d. column (for high pH runs), running at a flow rate of 20 mL/min with UV diode array detection (210-400 nm) and mass-directed collection using both positive and negative mass ion detection.

Alternatively, Preparative HPLC purification was carried out either on a Teledyne ISCO ACCQPrep® HP150 system or on a Waters Mass-directed PrepLC system, with a C1 XBridge BEH C18 (dimensions: 19 mm×150 mm 5 μm) or a C2 XBridge BEH C18 (dimensions: 19 mm×150 mm 5 μm) column, running a flow rate of 20 mL/min. All masses were detected with electrospray ionisation (ESI).

Purifications were performed using buffered acidic or basic solvent systems as appropriate. Compound retention times on the system were routinely assessed using a 30-50 μL test injection and a standard gradient, then purified using an appropriately chosen focused gradient as detailed below, based upon observed retention time.
Low pH:
Solvent A—Water+10 mM ammonium formate+0.1% formic acid
Solvent B—MeCN+5% water+0.1% formic acid
High pH:
Solvent A—Water+10 mM ammonium formate+0.1% ammonia solution
Solvent B—MeCN+5% water+0.1% ammonia solution or MeCN+0.1% ammonia solution

### Standard Gradient:

| | Flow rate | | |
|---|---|---|---|
| Time | (mL/min) | % Solvent A | % Solvent B |
| 0 | 20 | 90 | 10 |
| 0.3 | 20 | 90 | 10 |
| 8.5 | 20 | 2 | 98 |
| 12 | 20 | 2 | 98 |
| 12.5 | 0 | 2 | 98 |

### Focused Gradients:

| | Flow | % Solvent B | | | | |
|---|---|---|---|---|---|---|
| | rate | Retention time on standard | | | | |
| | (mL/ | gradient (min.) | | | | |
| Time | min) | 0-5.2 | 4.9-6.6 | 6.3-7.5 | 7.3-9.5 | 9.3-12 |
| 0 | 20 | 10 | 10 | 10 | 10 | 10 |
| 0.25 | 20 | 10 | 10 | 10 | 10 | 10 |
| 0.35 | 20 | 10 | 20 | 35 | 45 | 60 |
| 10 | 20 | 45 | 55 | 65 | 75 | 98 |
| 12 | 20 | 98 | 98 | 98 | 98 | 98 |
| 12.5 | 0 | 98 | 98 | 98 | 98 | 98 |

### Synthetic Methods

Several methods for the chemical synthesis of the compounds of the present invention are described herein. These and/or other well-known methods may be modified and/or adapted in known ways in order to facilitate the synthesis of additional compounds within the scope of the present invention.

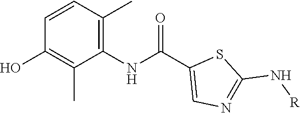

Amide coupling and deprotection to afford BAA-001

### 2-Amino-N-(3-hydroxy-2,6-dimethyl-phenyl)thiazole-5-carboxamide (BAA-001)

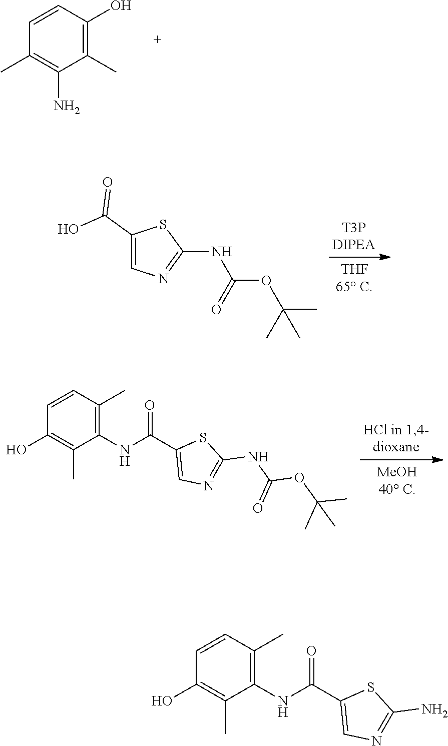

### Step 1: tert-Butyl N-[5-[(3-hydroxy-2,6-dimethyl-phenyl) carbamoyl]thiazol-2-yl]carbamate

To a stirring solution of 2-N-boc-amino-thiazole-5-carboxylic acid (350 mg, 1.43 mmol, 1.0 eq) and 3-amino-2,4-dimethyl-phenol (236 mg, 1.72 mmol, 1.2 eq), prepared as reported in International Patent Publication WO 2015/079251 A1, in THF (10 mL) was added DIPEA (0.50 mL, 2.87 mmol, 2.0 eq) and T3P (50% in EtOAc, 1.3 mL, 2.15 mmol, 1.5 eq). The mixture was heated to 65° C. and left to stir overnight. The mixture was cooled to RT, water and EtOAc added, and the layers separated. The aqueous was extracted with further EtOAc, the organics collected, passed through a phase separator and the solvents removed in vacuo. The crude material was chromatographed (SiO₂) eluting with 0-100% EtOAc: PE to afford tert-butyl N-[5-[(3-hydroxy-2,6-dimethyl-phenyl) carbamoyl]thiazol-2-yl]carbamate (64 mg, 0.176 mmol, 12%) as a pale orange solid. MS (ES+) m/z 364.2 (M+H). ¹H NMR (300 MHz, DMSO-d₆) δ 11.77 (br s, 1H), 9.64 (s, 1H), 9.18 (s, 1H), 8.17 (s, 1H), 6.89 (d, J=8.2 Hz, 1H), 6.69 (d, J=8.2 Hz, 1H), 2.06 (s, 3H), 1.98 (s, 3H), 1.51 (s, 9H).

### Step 2:2-Amino-N-(3-hydroxy-2,6-dimethyl-phenyl)thiazole-5-carboxamide (BAA-001)

To a stirring suspension of tert-butyl N-[5-[(3-hydroxy-2,6-dimethyl-phenyl) carbamoyl]thiazol-2-yl]carbamate (66 mg, 0.182 mmol, 1.0 eq) in MeOH(2 mL) was added

HCl (4M in 1,4-dioxane, 0.68 mL, 2.72 mmol, 15 eq). The mixture was warmed to 40° C. and stirred for 2 days. The solvents were removed in vacuo and chromatographed (SiO₂) using 0-100% EtOAc: PE followed by 0-20% MeOH: DCM. The resulting product was taken up in small amounts of MeOH and loaded onto a pre-equilibrated SCX-1 g column (washing with MeOH and eluting with 7N NH₃: MeOH) to afford the title compound (9.1 mg, 0.0346 mmol, 19%) as a pale yellow solid. MS (ES+) m/z 264.1 (M+H). ¹H NMR (300 MHz, DMSO-d₆) δ 9.32 (s, 1H), 9.13 (s, 1H), 7.83 (s, 1H), 7.53 (br s, 2H), 6.87 (d, J=8.2 Hz, 1H), 6.67 (d, J=8.2 Hz, 1H), 2.05 (s, 3H), 1.97 (s, 3H).

### Nitro Precursors Synthesis

### Nitration

### 3,4-Dimethyl-5-nitro-1H-pyrazole

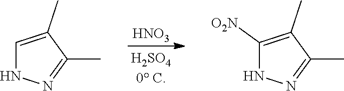

3,4-Dimethyl-1H-pyrazole (0.18 mL, 2.08 mmol, 1.0 eq) was added to sulfuric acid (95% in water, 5.0 mL, 88.6 mmol, 42.6 eq) at 0° C. then fuming nitric acid (0.13 mL, 3.12 mmol, 1.5 eq) added dropwise. The reaction stirred at RT for 18 h. The mixture was poured onto ice, added aq. ammonia solution to pH 4, extracted with EtOAc (x 3) and the combined organic layers dried (MgSO₄), filtered and concentrated in vacuo. The crude material was chromatographed (SiO₂) eluting with 0-100% EtOAc: PE to afford the title compound (60 mg, 0.425 mmol, 20%) as a white solid. MS (ES-) m/z 140.0 (M−H). ¹H NMR (300 MHz, DMSO-d₆) δ 13.62-13.42 (brs, 1H), 2.23 (s, 3H), 2.18 (s, 3H).

### Alkylation of Nitropyrazoles

### General method A1. Alkylation of 3-nitro-1H-pyrazoles

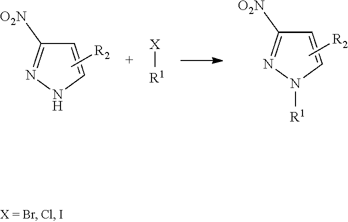

Alkyl halide (1 eq) is added dropwise to a suspension of unsubstituted or substituted nitropyrazole (1 eq) and potassium carbonate (1.5 eq) or caesium carbonate (1-1.5 eq) in anhydrous THF or MeCN, or NaH(1.2 eq) in DMF, with stirring at room temperature under nitrogen. The reaction mixture was then stirred at rt or heated to 65-75° C. with stirring for 4 hours, before cooling to room temperature and separating between ethyl acetate and water. The organic phase was washed with brine, dried over anhydrous magnesium sulfate, filtered and concentrated to give the product.

### 1-Cyclopentylpyrazol-3-amine

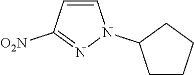 [SMILES: O=[N+]([O-])c1ccn(C2CCCC2)n1]

To a solution of 3-nitro-1H-pyrazole (1.0 g, 8.84 mmol, 1.0 eq) in DMF (25 mL) was added NaH(60% in mineral oil, 425 mg, 10.6 mmol, 1.2 eq) at RT, stirred for 10 min. then bromocyclopentane (0.95 mL, 8.84 mmol, 1.0 eq) was added. The mixture was stirred at RT for 12 h. The mixture was partitioned between EtOAc and water. The aqueous phase was extracted with EtOAc (3×50 mL). The combined organic phase was washed with water (2×50 mL), dried (MgSO₄) and concentrated in vacuo. The crude material was chromatographed (SiO₂) eluting with 0-100% EtOAc: PE to afford the title compound (850 mg, 4.69 mmol, 53%) as a white solid. MS (ES+) m/z 182.2 (M+H).

### Ethyl 2-methyl-5-nitro-pyrazole-3-carboxylate

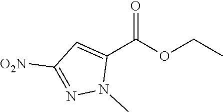

Prepared similarly from 5-nitro-1H-pyrazole-3-carboxylic acid ethyl ester (1.0 g, 5.40 mmol, 1.0 eq), NaH(60% in mineral oil, 238 mg, 5.94 mmol) and iodomethane (12 mL, 191 mmol, 3.0 eq) to afford the title compound (540 mg, 2.71 mmol, 50%) as a white solid after reverse phase chromatography (C18) eluting with 5-95% MeCN: H₂O which was used in next step without further purification. ¹H NMR (300 MHz, Chloroform-d) δ 7.37 (s, 1H), 4.39 (q, J=7.1 Hz, 2H), 4.26 (s, 3H), 1.39 (t, J=7.1 Hz, 3H).

### 3-Nitro-1-(2,2,2-trifluoroethyl)pyrazole

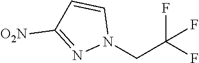 [SMILES: O=[N+]([O-])c1ccn(CC(F)(F)F)n1]

A mixture of 3-nitro-1H-pyrazole (0.70 g, 6.19 mmol, 1.0 eq) and Cs₂CO₃ (4.0 g, 12.4 mmol, 2.0 eq) in DMF (25 mL) was stirred for 5 min. then 2,2,2-trifluoroethyl trifluoromethanesulfonate (1.72 mg, 7.43 mmol, 1.2 eq) was added and the mixture stirred for 18 h at RT. The mixture was poured onto water (100 mL) and extracted with EtOAc (3×25 mL). The combined organic phase was washed with water, dried (MgSO₄) and concentrated in vacuo to afford the title compound (850 mg, 4.36 mmol, 70%) as a white solid. ¹H NMR (300 MHz, DMSO-d₆) δ 8.16 (d, J=2.6 Hz, 1H), 7.16 (d, J=2.6 Hz, 1H), 5.38 (q, J=9.0 Hz, 2H). 19F NMR (282 MHz, DMSO-d₆) δ-70.11 (t, J=9.0 Hz).

### 3-Nitro-1-(oxetan-3-yl)pyrazole

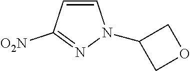 [SMILES: O=[N+]([O-])c1ccn(C2COC2)n1]

Prepared similarly from 3-iodooxetane (1.06 g, 5.75 mmol), 3-nitro-1H-pyrazole (0.65 g, 5.75 mmol) and Cs₂CO₃ (2.81 g, 8.62 mmol) at 100° C. for 16 h to afford the title compound (534 mg, 3.16 mmol, 55%) after normal phase chromatography (SiO₂) eluting with 0-100% EtOAc: PE. MS (ES-) m/z 168.0 (M−H). ¹H NMR (300 MHz, Chloroform-d) δ 7.73 (d, J=2.6 Hz, 1H), 6.97 (d, J=2.5 Hz, 1H), 5.62-5.46 (m, 1H), 5.15-4.95 (m, 4H).

### tert-Butyl N-[2-(3-nitropyrazol-1-yl)ethyl]carbamate

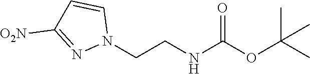 [SMILES: CC(C)(C)OC(=O)NCCn1ccc([N+](=O)[O-])n1]

Prepared similarly from tert-butyl N-(2-bromoethyl)carbamate (2.18 g, 9.73 mmol), 3-nitro-1H-pyrazole (1.00 g, 8.84 mmol) and Cs₂CO₃ (3.46 g, 10.6 mmol) at 60° C. for 12 h to afford the title compound (1.3 g, 5.07 mmol, 57%) as a beige solid after normal phase chromatography (SiO₂) eluting with 0-100% EtOAc: PE. MS (ES-) m/z 255.1 (M−H).

### 1-(Difluoromethyl)-3-nitro-pyrazole

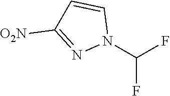 [SMILES: O=[N+]([O-])c1ccn(C(F)F)n1]

Prepared similarly from sodium chlorodifluoroacetate (5.39 g, 35.38 mmol, 2.0 eq), 3-Nitro-1H-pyrazole (2.00 g, 17.7 mmol), cesium carbonate (5.76 g, 17.7 mmol) at 120° C. for 2 h to afford the title compound (2.0 g, 12.3 mmol, 69%) as a white solid after normal phase chromatography (SiO₂) eluting with 0-10% EtOAc: PE. ¹H NMR (300 MHz, Chloroform-d) δ 7.98 (d, J=2.8 Hz, 1H), 7.29 (s, 1H), 7.09 (d, J=2.5 Hz, 1H). 19F NMR (282 MHz, Chloroform-d) 0-95.11 (d, J=59.7 Hz).

### 3-(3-Nitropyrazol-1-yl)propan-1-ol

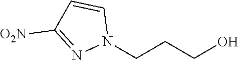 [SMILES: O=[N+]([O-])c1ccn(CCCO)n1]

To a solution of 3-nitro-1H-pyrazole (2.30 g, 20.3 mmol, 1.0 eq) in THF (65 mL) was added 3-chloro-1-propanol (1.8 mL, 21.4 mmol, 1.05 eq) and K₂CO₃ (5.62 g, 40.7 mmol, 2.0 eq). The mixture was heated to 60° C. for 72 h, followed by 75° C. for 18 h. The mixture was partitioned between EtOAc and water. The aqueous phase was extracted with EtOAc (3×50 mL). The combined organic phase was washed with water (2×50 mL), dried (MgSO₄), and concentrated under reduced pressure. The residue was chromatographed (SiO₂) eluting with 0-100% EtOAc: PE to afford the title compound (2.9 g, 17.2 mmol, 84%) as a colourless oil. MS (ES+) m/z 172.2 (M+H). ¹H NMR (300 MHz, Chloroform-d) δ 7.53 (d, J=2.4 Hz, 1H), 6.91 (d, J=2.5 Hz, 1H), 4.40 (t, J=6.8 Hz, 2H), 3.74-3.60 (m, 2H), 2.28-2.06 (m, 2H).

### Ethyl 4-(3-nitropyrazol-1-yl)butanoate

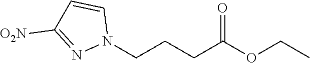 [SMILES: CCOC(=O)CCCn1ccc([N+](=O)[O-])n1]

Prepared similarly from ethyl 4-bromobutyrate (2.55 mL, 17.69 mmol), 3-nitro-1H-pyrazole
(2.00 g, 17.7 mmol) and K₂CO₃ (4.89 g, 35.4 mmol) to afford the title compound (3.7 g, 16.3 mmol, 92%) as a colourless oil after normal phase chromatography (SiO₂) eluting with 0-100% EtOAc: PE. ¹H NMR (300 MHz, Chloroform-d) o 7.49 (d, J=2.5 Hz, 1H), 6.92-6.73 (m, 1H), 4.21 (t, J=6.8 Hz, 2H), 3.99 (q, J=7.1 Hz, 2H), 2.33-2.03 (m, 4H), 1.12 (t, J=7.1 Hz, 3H).

### N, N-Dimethyl-2-(3-nitropyrazol-1-yl)acetamide

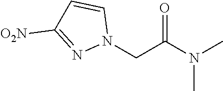

Prepared similarly from 2-bromo-N, N-dimethylacetamide (0.33 mL, 3.10 mmol, 1 eq), 3-nitro-1H-pyrazole (350 mg, 3.10 mmol) and K₂CO₃ (868 mg, 6.19 mmol) at 70° C. for 2 h to afford the title compound (400 mg, 2.02 mmol, 65%) as a white solid after normal phase chromatography (SiO₂) eluting with 0-20% MeOH: DCM. MS (ES-) m/z 197.1 (M−H). ¹H NMR (300 MHz, Chloroform-d) δ 7.64 (d, J=2.6 Hz, 1H), 6.97 (d, J=2.5 Hz, 1H), 5.10 (s, 2H), 3.12 (s, 3H), 3.02 (s, 3H).

### Ethyl 2-(5-methyl-3-nitro-pyrazol-1-yl)acetate

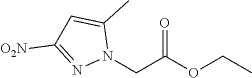

Prepared similarly from 5-methyl-3-nitro-1H-pyrazole (1.0 g, 7.87 mmol), K₂CO₃ (2.2 g, 15.7 mmol) using ethyl bromoacetate (0.87 mL, 7.87 mmol, 1.0 eq) with addition at 0° C., followed by heating at 70° C. for 2 h to afford the title compound (1.62 g, 7.60 mmol, 97%) as a white solid. MS (ES-) m/z 211.9 (M−H). ¹H NMR (300 MHz, DMSO-d₆) δ 6.92 (d, J=0.8 Hz, 1H), 5.26 (s, 2H), 4.20 (q, J=7.1 Hz, 2H), 2.29 (d, J=0.7 Hz, 3H), 1.23 (t, J=7.1 Hz, 3H).

### 1,4-Dimethyl-3-nitro-pyrazole

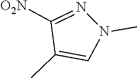

Prepared similarly from 4-methyl-3-nitro-1H-pyrazole (300 mg, 2.36 mmol, 1.0 eq), K₂CO₃
(662 mg, 4.72 mmol) using iodomethane (0.18 mL, 2.83 mmol, 1.2 eq) with addition at 0° C., followed by heating at 70° C. for 2 h to afford the title compound (270 mg, 1.91 mmol, 81%) as a white solid after normal phase chromatography (SiO₂) eluting with 0-100% EtOAc: PE. MS (ES+) m/z 142.1 (M+H). ¹H NMR (300 MHz, DMSO-d₆) δ 7.82 (d, J=1.0 Hz, 1H), 3.91 (s, 3H), 2.24 (d, J=0.8 Hz, 3H).

### Ethyl 2-(4-methyl-3-nitro-pyrazol-1-yl)acetate

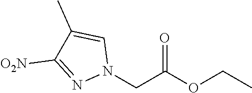

Prepared similarly from 4-methyl-3-nitro-1H-pyrazole (500 mg, 3.93 mmol) and K₂CO₃ (1.1 g, 7.87 mmol) using ethyl bromoacetate (0.44 mL, 3.93 mmol, 1.0 eq) with addition at 0° C., followed by heating at 70° C. for 2 h to afford the title compound (820 mg, 3.85 mmol, 98%) as a light yellow oil. MS (ES-) m/z 212.1 (M−H). ¹H NMR (300 MHz, DMSO-d₆) δ 7.88 (d, J=0.9 Hz, 1H), 5.22 (s, 2H), 4.18 (q, J=7.1 Hz, 2H), 2.27 (d, J=0.8 Hz, 3H), 1.23 (t, J=7.1 Hz, 3H).

### 1-Methyl-3-nitro-indazole

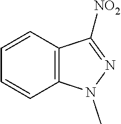

Prepared similarly from 3-nitro-1H-indazole (255 mg, 1.56 mmol) and K₂CO₃ (438 mg, 3.13 mmol) using iodomethane (0.12 mL, 1.88 mmol, 1.2 eq) with addition at 0° C., followed by heating at 70° C. for 2 h to afford the title compound (220 mg, 1.24 mmol, 79%) as a light yellow solid after normal phase chromatography (SiO₂) eluting with 0-100% EtOAc: PE. MS (ES+) m/z 178.0 (M+H). ¹H NMR (300 MHz, Chloroform-d) δ 8.31-8.20 (m, 1H), 7.63-7.43 (m, 3H), 4.22 (s, 3H).

### 1,4,5-Trimethyl-3-nitro-pyrazole

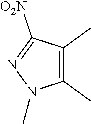

Prepared similarly from 3,4-dimethyl-5-nitro-1H-pyrazole (80 mg, 0.567 mmol, 1.0 eq) and K₂CO₃ (159 mg, 1.13 mmol) using iodomethane (0.042 mL, 0.680 mmol, 1.2 eq) with addition at 0° C., followed by heating at 70° C. for 2 h to afford the title compound (60 mg, 0.387 mmol, 68%) as a white solid after normal phase chromatography (SiO₂) eluting with 0-100% EtOAc: PE. MS (ES+) m/z 155.9 (M+H). ¹H NMR (300 MHz, Chloroform-d) δ 3.83 (s, 3H), 2.23 (s, 3H), 2.22 (s, 3H).

The following examples were prepared in a similar manner using method A1 with the appropriate nitro-pyrazole and alkyl halide:

| Structure | Name | Analytical Data |
|---|---|---|
| 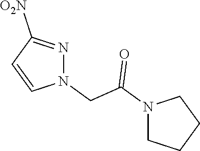 [SMILES: O=C(Cn1ccc([N+](=O)[O-])n1)N1CCCC1] | 2-(3-nitropyrazol- 1-yl)-1-pyrrolidin- 1-yl-ethanone | MS (ES+) m/z = 225.1 [M + H]+; ¹H NMR (400 MHz, CHLOROFORM-D) δ 7.67 (d, J = 2.5 Hz, 1H), 6.94 (d, J = 2.5 Hz, 1H), 4.99 (s, 2H), 3.51 (td, J = 6.9, 2.7 Hz, 4H), 2.10-1.98 (m, 2H), 1.96-1.84 (m, 2H). |
| 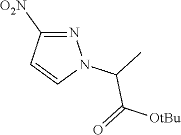 [SMILES: CC(C(=O)OC(C)(C)C)n1ccc([N+](=O)[O-])n1] | tert-butyl 2-(3- nitro-1H-pyrazol- 1-yl)propanoate | MS (ES−) m/z = 240.2 [M − H]−, ¹H NMR (400 MHz, DMSO-D6) δ 8.14 (d, J = 2.6 Hz, 1H), 7.10 (d, J = 2.6 Hz, 1H), 5.35 (q, J = 7.3 Hz, 1H), 1.70 (d, J = 7.3 Hz, 3H), 1.39 (s, 9H). |
| 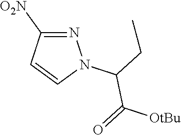 [SMILES: CCC(C(=O)OC(C)(C)C)n1ccc([N+](=O)[O-])n1] | tert-butyl 2-(3- nitro-1H-pyrazol- 1-yl)butanoate | MS (ES−) m/z = 254.2 [M − H]−, ¹H NMR (400 MHz, DMSO-D6) δ 8.14 (d, J = 2.6 Hz, 1H), 7.11 (d, J = 2.6 Hz, 1H), 5.14 (dd, J = 9.0, 6.3 Hz, 1H), 2.21-2.09 (m, 2H), 1.39 (s, 9H), 0.81 (t, J = 7.4 Hz, 3H). |
| 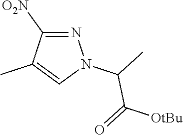 [SMILES: Cc1cn(C(C)C(=O)OC(C)(C)C)nc1[N+](=O)[O-]] | tert-butyl 2-(4- methyl-3-nitro- 1H-pyrazol-1-yl) propanoate | MS (ES−) m/z = 254.2 [M − H]−, 1H NMR (400 MHz, CHLOROFORM-D) δ 7.45 (d, J = 1.0 Hz, 1H), 5.01 (q, J = 7.4 Hz, 1H), 2.36 (d, J = 0.9 Hz, 3H), 1.77 (d, J = 7.4 Hz, 3H), 1.45 (s, 9H). |
| 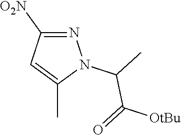 [SMILES: Cc1cc([N+](=O)[O-])nn1C(C)C(=O)OC(C)(C)C] | tert-butyl 2-(5- methyl-3-nitro- 1H-pyrazol-1-yl) propanoate | MS (ES−) m/z = 254.1 [M − H]⁻; ¹H NMR (400 MHz, DMSO-D6) δ 6.90 (q, J = 0.8 Hz, 1H), 5.36 (q, J = 7.1 Hz, 1H), 2.31 (d, J = 0.8 Hz, 3H), 1.66 (d, J = 7.1 Hz, 3H), 1.37 (d, J = 0.9 Hz, 9H). |
| 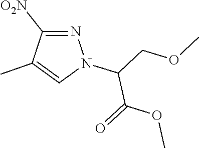 | methyl 3-meth- oxy-2-(4-methyl- 3-nitro-1H- pyrazol-1-yl) propanoate | MS (ES+) m/z = 230.1, [M + H]+; 1H NMR (400 MHz, CDCl3) δ 7.82 (d, J = 2.6 Hz, 1H), 6.93 (d, J = 2.6 Hz, 1H), 5.28 (dd, J = 5.3, 3.0 Hz, 1H), 4.16 (dd, J = 10.3, 5.3 Hz, 1H), 3.92 (dd, J = 10.3, 3.0 Hz, 1H), 3.80 (s, 3H), 3.38 (s, 3H). |
| 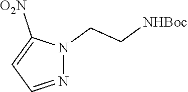 [SMILES: CC(C)(C)OC(=O)NCCn1nccc1[N+](=O)[O-]] | tert-butyl (2-(5- nitro-1H- pyrazol-1-yl)- ethyl)carbamate | MS (ES+) m/z 157.5 [M − Boc + H]⁺. ¹H NMR (300 MHz, DMSO) δ 7.67 (d, J = 2.2 Hz, 1H), 7.24 (d, J = 2.2 Hz, 1H), 6.90 (t, J = 6.1 Hz, 1H), 4.56 (dd, J = 6.3, 5.3 Hz, 2H), 3.40-3.32 (m, 2H), 1.30 (s, 9H). |
| 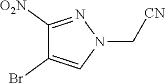 [SMILES: N#CCn1cc(Br)c([N+](=O)[O-])n1] | 2-(4-bromo-3- nitro-1H-pyrazol- 1-yl)acetonitrile | ¹H NMR (400 MHz, DMSO-d6) δ 8.44 (s, 1H), 5.68 (s, 2H) |
| 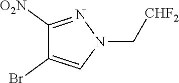 [SMILES: O=[N+]([O-])c1nn(CC(F)F)cc1Br] | 4-bromo-1-(2,2- difluoroethyl)- 3-nitro-1H- pyrazole | ¹H NMR (400 MHz, DMSO-d6) δ 7.67 (s, 1H), 6.15 (ttd, J = 54.7, 4.1, 1.2 Hz, 1H), 4.55 (td, J = 13.4, 4.0 Hz, 1H). |
| 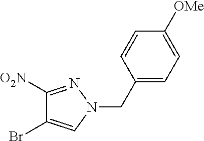 [SMILES: COc1ccc(Cn2cc(Br)c([N+](=O)[O-])n2)cc1] | 4-bromo-1-(4- methoxybenzyl)- 3-nitro-1H- pyrazole | 1H NMR (400 MHz, CDCl3) δ 7.38 (s, 1H), 7.26 (d, J = 8.7 Hz, 2H), 6.93 (d, J = 8.7 Hz, 2H), 5.27 (s, 2H), 3.82 (s, 3H). |

### 2-(3-Nitro-4-vinyl-1H-pyrazol-1-yl)acetonitrile

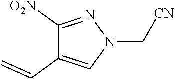

A solution of potassium vinyltrifluoroborate (175 mg, 1.31 mmol), palladium (II) chloride (12 mg, 0.0677 mmol), triphenylphosphine (52 mg, 0.198 mmol), caesium carbonate (640 mg, 1.96 mmol), and 2-(4-bromo-3-nitro-pyrazol-1-yl)acetonitrile (150 mg, 0.649 mmol) in a mixture of 1,4-dioxane (3 mL) and water (0.3 mL) was degassed with N₂ and stirred at 85° C. in a sealed tube for 6 h. The reaction was cooled to rt and quenched with water (15 mL). The resulting mixture was extracted with DCM (3×15 mL), and the combined organics were dried over Na₂SO₄, filtered and concentrated. The crude material was purified by column chromatography over silica (40 g cartridge) eluting with a gradient of ethyl acetate (5% to 80%; v/v) in iso-hexane to afford the title compound (67 mg, 0.342 mmol, 52.63% yield) as a yellow solid. ¹H NMR (400 MHz, CDCl₃) δ 7.84 (s, 1H), 7.04 (ddd, J=17.8, 11.1, 0.7 Hz, 1H), 5.69 (dd, J=17.8, 0.9 Hz, 1H), 5.47 (dd, J=11.0, 0.8 Hz, 1H), 5.16 (s, 2H).

### 1-(2.2-Difluoroethyl)-3-nitro-4-vinyl-1H-pyrazole

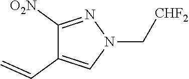

A solution of potassium vinyltrifluoroborate (50 mg, 0.373 mmol), palladium (II) chloride (3.5 mg, 0.0195 mmol), triphenylphosphine (15 mg, 0.0586 mmol), caesium carbonate (200 mg, 0.614 mmol), and 4-bromo-1-(2,2-difluoroethyl)-3-nitro-pyrazole (50 mg, 0.195 mmol) in a mixture of 1,4-Dioxane (1 mL) and Water (0.1 mL) was degassed with N₂ and stirred at 85° C. in a sealed tube for 6 h. The reaction was quenched with water (5 mL) and was extracted with DCM (3×5 mL), and the combined organics were dried over Na₂SO₄, filtered and concentrated. The crude material was purified by column chromatography over silica (12 g cartridge) eluting with a gradient of Ethyl acetate (5% to 80%; v/v) in iso-hexane to afford the desired product 1-(2,2-difluoroethyl)-3-nitro-4-vinyl-pyrazole (33 mg, 0.149 mmol, 76.29% yield) as a yellow solid. ¹H NMR (400 MHz, CDCl₃) δ 7.71 (s, 1H), 7.03 (ddd, J=17.8, 11.1, 0.7 Hz, 1H), 6.16 (tt, J=54.9, 4.1 Hz, 1H), 5.65 (dd, J=17.8, 1.0 Hz, 1H), 5.42 (dd, J=11.1, 1.0 Hz, 1H), 4.53 (td, J=13.4, 4.1 Hz, 2H). 19F NMR (376 MHz, CDCl₃) 0-122.32 (dt, J=54.8, 13.3 Hz).

### 1-(2-Fluoroethyl)-3-nitro-4-vinyl-1H-pyrazole

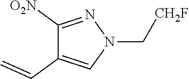

A solution of potassium vinyltrifluoroborate (170 mg, 1.27 mmol), palladium (II) chloride (11 mg, 0.0620 mmol), triphenylphosphine (50 mg, 0.191 mmol), caesium carbonate (620 mg, 1.90 mmol), and 4-bromo-1-(2-fluoroethyl)-3-nitro-pyrazole (150 mg, 0.630 mmol) in a mixture of 1,4-dioxane (3 mL) and water (0.3 mL) was degassed with N₂ and stirred at 85° C. in a sealed tube for 2 days. The reaction was cooled to rt and quenched with water (5 mL). The resulting mixture was extracted with DCM (3×5 mL), and the combined organics were dried over Na₂SO₄, filtered and concentrated. The crude material was purified by column chromatography over silica (12 g cartridge) eluting with a gradient of Ethyl acetate (5% to 80%; v/v) in iso-hexane to afford the title compound (111 mg, 0.535 mmol, 84.89% yield) as a yellow solid. ¹H NMR (400 MHz, CDCl₃) δ 7.71 (s, 1H), 7.06 (dd, J=17.8, 11.1 Hz, 1H), 5.64 (dd, J=17.8, 1.0 Hz, 1H), 5.40 (dd, J=11.1, 1.0 Hz, 1H), 4.91-4.71 (m, 2H), 4.47 (dt, J=26.9, 4.5 Hz, 2H). 19F NMR (376 MHz, CDCl₃) δ-221.93 (tt, J=46.9, 27.0 Hz).

### 1-(4-Methoxybenzyl)-3-nitro-4-vinyl-1H-pyrazole

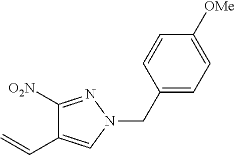 [SMILES: C=Cc1cn(Cc2ccc(OC)cc2)nc1[N+](=O)[O-]]

A solution of potassium vinyltrifluoroborate (130 mg, 0.971 mmol), palladium (II) chloride
(9.0 mg, 0.0508 mmol), triphenylphosphine (40 mg, 0.153 mmol), caesium carbonate (480 mg, 1.47 mmol), and 4-bromo-1-[(4-methoxyphenyl)methyl]-3-nitro-pyrazole (260 mg, 0.491 mmol) in a mixture of 1,4-dioxane (2.5 mL) and water (0.25 mL) was degassed with N₂ and stirred at 85° C. in a sealed tube overnight. The reaction was quenched with water (5 mL), extracted with DCM (3×5 mL), and the combined organics were dried over Na₂SO₄, filtered and concentrated. The crude material was purified by column chromatography over silica (24 g cartridge) eluting with a gradient of DCM (50% to 100%; v/v) in iso-hexane to afford the title compound (143 mg, 0.474 mmol, 96.45% yield) as a yellow solid. ¹H NMR (400 MHz, CDCl₃) δ 7.46 (s, 1H), 7.27 (d, J=9.1 Hz, 2H), 7.03 (dd, J=17.8, 11.1 Hz, 1H), 6.92 (d, J=8.7 Hz, 2H), 5.53 (dd, J=17.8, 1.1 Hz, 1H), 5.32 (dd, J=11.1, 1.1 Hz, 1H), 5.26 (s, 2H), 3.82 (s, 3H).

### 4-Methyl-3-nitro-1-(2,2,2-trifluoroethyl)-1H-pyrazole

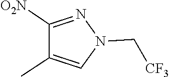

Prepared similarly from sodium hydride (60% in mineral oil, 0.19 g, 4.72 mmol, 1.20 eq), 4-methyl-3-nitro-1H-pyrazole (0.50 g, 3.93 mmol, 1.00 eq), DMF (10 mL), 1-lodo-2,2,2-trifluoroethane
(0.42 mL, 4.33 mmol, 1.10 eq) at 60° C. for 48 hours to afford the title compound after chromatography (0-100% EtOAc in PE) as a white powder (184 mg, 0.877 mmol, 22%). ¹H NMR
(300 MHz, CDCl₃) δ 7.46 (s, 1H), 4.76 (q, J=8.1 Hz, 2H), 2.40 (d, J=0.8 Hz, 3H). ¹⁹F NMR (282 MHz, CDCl₃) 0-71.21. MS (ES+) m/z 208.0 [M−H]⁻.

### tert-Butyl 3-(4-methyl-3-nitro-1H-pyrazol-1-yl) azetidine-1-carboxylate

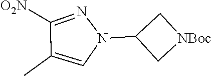 [SMILES: Cc1cn(C2CN(C(=O)OC(C)(C)C)C2)nc1[N+](=O)[O-]]

Prepared similarly from N-boc-3-iodoazetidine (0.51 mL, 2.95 mmol, 1.50 eq), cesium carbonate (1.29 g, 3.93 mmol, 2.00 eq), 4-methyl-3-nitro-1H-pyrazole (0.25 g, 1.97 mmol, 1.00 eq) and DMF (4 mL) at 70° C. overnight to afford the title compound after chromatography (0-100% EtOAc in PE) as a yellow powder (400 mg, 1.42 mmol, 72%). ¹H NMR (300 MHz, MeOD) δ 7.78 (d, J=0.9 Hz, 1H), 5.23 (tt, J=7.9, 5.2 Hz, 1H), 4.47-4.35 (m, 2H), 4.34-4.24 (m, 3H), 2.34 (d, J=0.9 Hz, 3H), 1.49 (s, 9H).

### 1-Cyclobutyl-4-methyl-3-nitro-1H-pyrazole

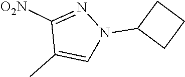

Prepared similarly from cesium carbonate (1.29 g, 3.93 mmol, 2.00 eq), bromocyclobutane
(0.56 mL, 5.90 mmol, 3.00 eq), 4-methyl-3-nitro-1H-pyrazole (0.25 g, 1.97 mmol, 1.00 eq) and DMF (4 mL) at 100° C. overnight to afford the title compound after chromatography (0-100% EtOAc in PE) as a yellow solid (294 mg, 1.62 mmol, 83%). ¹H NMR (300 MHz, DMSO) δ 7.99 (d, J=0.9 Hz, 1H), 5.04-4.82 (m, 1H), 2.52-2.32 (m, 4H), 2.26 (d, J=0.9 Hz, 3H), 1.92-1.72 (m, 2H). MS (ES+) m/z 181.9 [M+H]⁺.

### 4-Methyl-3-nitro-1-(oxetan-3-yl)-1H-pyrazole

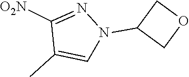

Prepared similarly from cesium carbonate (1.29 g, 3.93 mmol, 2.00 eq), 3-bromooxetane
(0.49 mL, 5.90 mmol, 3.00 eq), 4-methyl-3-nitro-1H-pyrazole (0.25 g, 1.97 mmol, 1.00 eq) and DMF (4 mL) at 100° C. overnight to afford the title compound after chromatography (0-100% EtOAc in PE) as a white powder (213 mg, 1.16 mmol, 59%). ¹H NMR (300 MHz, DMSO) δ 8.07 (q, J=0.9 Hz, 1H), 5.72-5.57 (m, 1H), 5.01-4.90 (m, 2H), 4.90-4.82 (m, 2H), 2.28 (d, J=0.8 Hz, 3H). MS (ES-) m/z 182.1 [M-H]—

### 2-[(4-Methyl-3-nitro-pyrazol-1-yl)methyl]oxazole

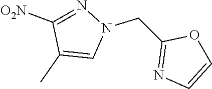

To a mixture of 2-hydroxymethyl oxazole (125 mg, 1.26 mmol, 1 eq) and triethylamine
(0.23 mL, 1.64 mmol, 1.3 eq) in CH₂Cl₂ (6 mL) at 0° C. was added dropwise methanesulfonyl chloride (0.12 mL, 1.58 mmol, 1.25 eq) and the reaction stirred at 0° C. for 1 h. CH₂Cl₂ (5 mL) and water (5 mL) were added, the phases were separated and the aqueous phase extracted with CH₂Cl₂ (2×5 mL). Combined organic phases were washed with NH₄Cl (10 mL, 10% w/w aqueous solution), brine (20 mL), dried over MgSO₄, filtered and concentrated in vacuo. This crude material was dissolved in acetone (6 mL). 4-methyl-3-nitro-1H-pyrazole (160 mg, 1.26 mmol, 1 eq), potassium carbonate (872 mg, 6.31 mmol, 5 eq) and tetrabutylammonium bromide (81 mg, 0.252 mmol, 0.2 eq) were added and the reaction stirred at rt until completion. The solvent was removed under reduced pressure. Ethyl acetate (5 mL) and water (5 mL) were added. The phases were separated and the aqueous phase extracted with ethyl acetate (2×5 mL). Combined organic phases were washed with brine (20 mL), dried over MgSO₄, filtered and concentrated in vacuo. Column chromatography (petroleum ether:ethyl acetate) gave 2-[(4-methyl-3-nitro-pyrazol-1-yl)methyl]oxazole (186 mg, 0.893 mmol, 71%). MS (ES+) m/z 209 (M+H). ¹H NMR (300 MHz, Chloroform-d) δ 7.67 (d, J=0.9 Hz, 1H), 7.44 (q, J=0.9 Hz, 1H), 7.12 (d, J=0.8 Hz, 1H), 5.45 (s, 2H), 2.31 (d, J=0.9 Hz, 3H).

### 1-(4-Methyl-3-nitro-1H-pyrazol-1-yl)propan-2-ol

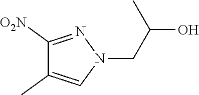

To a solution of 4-methyl-5-nitro-1H-pyrazole (300 mg, 2.36 mmol) in MeCN (23.6 mL), were added potassium; carbonate (936 mg, 9.44 mmol) and 1-chloro-2-propanol (0.86 mL, 7.08 mmol). The mixture was stirred at 70° C. overnight. Additional 1.5 eq of 1-chloro-2-propanol (0.43 mL) was added and the stirring continued at 80° C. for 5 h. The mixture was treated with water (20 ml) and EtOAc (30 mL) and then CHCl3: IPA (3:1)(2×30 ml). The organic phase was separated, dried over Na₂SO₄ and concentrated under reduce pressure. The product was used in the next step without purification. MS (ES+) m/z=186.1 [M+H]+, H NMR (400 MHz, CHLOROFORM-D) δ 7.38 (q, J=0.9 Hz, 1H), 4.86 (ddd, J=7.7, 7.2, 6.2 Hz, 1H), 4.55 (dd, J=8.4, 7.7 Hz, 1H), 4.20 (dd, J=13.8, 3.0 Hz, 1H), 4.03 (dd, J=4.6, 3.4 Hz, 1H), 2.34 (d, J=0.9 Hz, 3H), 1.26 (d, J=6.3 Hz, 3H).

### 1-(2-((tert-Butyldimethylsilyl)oxy)propyl)-4-methyl-3-nitro-1H-pyrazole

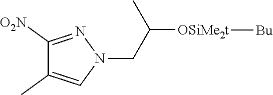

To a solution of 1-(4-methyl-3-nitro-pyrazol-1-yl)propan-2-ol (123 mg, 0.664 mmol) and imidazole (99 mg, 1.46 mmol) in DCM (6.64 mL) was added tert-butyldimethylchlorosilane (400 mg, 2.66 mmol) at rt. The reaction was stirred at 22° C. for 16 h, then was treated with NaHCO₃ (20 mL), water (20 mL) and DCM (50 ml). The organic phase was separated, dried over Na₂SO₄ and concentrated under reduce pressure to afford the desired product tert-butyl-dimethyl-[1-methyl-2
(4-methyl-3-nitro-pyrazol-1-yl) ethoxy]silane (166 mg, 0.543 mmol, 81.79% yield) as a yellow oil. MS (ES+) m/z=300.1 [M+H]+. ¹H NMR (400 MHz, CHLOROFORM-D) δ 7.29 (q, J=0.8 Hz, 1H), 4.21 (tdd, J=6.1, 3.0, 2.1 Hz, 1H), 4.15 (dd, J=13.5, 3.0 Hz, 1H), 3.91 (dd, J=13.5, 8.3 Hz, 1H), 2.33 (d, J=0.8 Hz, 3H), 1.18 (d, J=6.2 Hz, 3H), 0.81 (s, 9H), -0.03 (s, 3H), -0.24 (s, 3H).

### 3-(5-Methyl-3-nitro-1H-pyrazol-1-yl)tetrahydrothiophene 1.1-dioxide

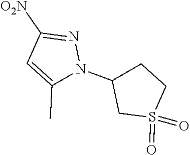 [SMILES: Cc1cc([N+](=O)[O-])nn1C1CCS(=O)(=O)C1]

To a solution of 5-methyl-3-nitro-1H-pyrazole (375 mg, 2.95 mmol) in MeCN (25 mL) was added potassium carbonate 325 mesh (1265 mg, 9.15 mmol) and (1,1-dioxothiolan-3-yl) methanesulfonate (719 mg, 2.95 mmol). The reaction mixture was stirred at 50° C. for 18 h and at 75° C. for 24 h, then it was quenched with saturated aqueous NH₄Cl (50 mL), filtered, washed with water and i-hexane and dried to afford the title compound (319 mg, 1.30 mmol, 44% yield) as a white solid. MS (ES+) m/z=246.0 [M+H]+. ¹H NMR (400 MHz, DMSO-D6) δ 6.91 (q, J=0.7 Hz, 1H), 5.49-5.27 (m, 1H), 3.80 (dd, J=13.7, 8.7 Hz, 1H), 3.55-3.45 (m, 1H), 3.42-3.33 (m, 1H), 3.32-3.22 (m, 1H), 2.72-2.60 (m, 1H), 2.56-2.52 (m, 1H), 2.39 (d, J=0.8 Hz, 3H).

### Sulphonylation of nitro-pyrazoles

### 1-(Isopropylsulfonyl)-4-methyl-3-nitro-1H-pyrazole

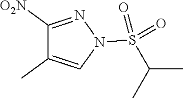

Iso-propylsulfonyl chloride (0.42 mL, 3.78 mmol, 1.20 eq) was added to a solution of 4-methyl-3-nitro-1H-pyrazole (0.40 g, 3.15 mmol, 1.00 eq) and triethylamine (0.53 mL, 3.78 mmol, 1.20 eq) in anhydrous DCM (4 mL) at 0° C. The reaction mixture was warmed to room temperature and allowed to stir for 2 hours. It was diluted with DCM and sat. NaHCO₃. The phases were separated, and the aqueous phase was further extracted with DCM (2×). The combined organic extracts were washed with water and brine. They were dried over anhydrous MgSO₄, filtered and concentrated in vacuo to afford the title compound (710 mg, 3.04 mmol, 97%) as a yellow oil. 1H NMR (300 MHz,) δ 8.50 (q, J=1.0 Hz, 1H), 4.09 (hept, J=6.8 Hz, 1H), 2.31 (d, J=1.0 Hz, 3H), 1.30 (d, J=6.8 Hz, 6H). MS (ES+) m/z 232.0 [M-H]−.

### Preparation of nitropyrazole acetamides from esters

### Conversion of ethyl 2-(3-nitropyrazol-1-yl)acetate to amide

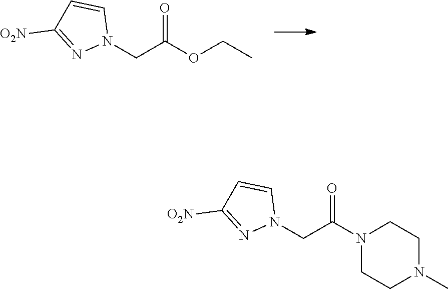 [SMILES: CCOC(=O)Cn1ccc([N+](=O)[O-])n1 ; CN1CCN(C(=O)Cn2ccc([N+](=O)[O-])n2)CC1]

### 1-(4-Methylpiperazin-1-yl)-2-(3-nitropyrazol-1-yl)ethenone

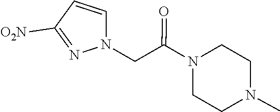 [SMILES: CN1CCN(C(=O)Cn2ccc([N+](=O)[O-])n2)CC1]

To a solution of ethyl 2-(3-nitropyrazol-1-yl)acetate (1.0 g, 5.02 mmol, 1.0 eq) in 1,4-dioxane (25 mL) was added N-methylpiperazine (3.0 mL, 27.0 mmol, 5.4 eq) and the mixture stirred at 100° C. for 72 h. The mixture was cooled, concentrated in vacuo and the residue chromatographed (SiO₂) eluting with 0-40% MeOH: DCM to afford the title compound (700 mg, 2.76 mmol, 55%) as a yellow solid. MS (ES+) m/z 254.3 (M+H).

### 3-Methoxy-2-(4-methyl-3-nitro-pyrazol-1-yl)-1-[(2R)-2-(trifluoromethyl)pyrrolidin-1-yl]propan-1-one

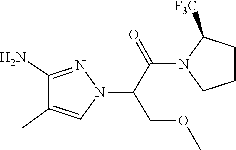 [SMILES: COCC(C(=O)N1CCC[C@@H]1C(F)(F)F)n1cc(C)c(N)n1]

To a solution of cesium 3-methoxy-2-(4-methyl-3-nitro-pyrazol-1-yl)propanoate (1.20 g, 3.32 mmol), DIPEA (1.8 mL, 10.0 mmol) and HATU (1.50 g, 3.94 mmol) in DMF (20 mL) was added (2R)-2-(trifluoromethyl) pyrrolidine (650 mg, 4.67 mmol). The reaction was stirred at rt overnight, then it was diluted with EtOAc (100 mL) and washed with aq. LiCl 1M (150 mL). The aqueous solution was extracted with EtOAc (150 mL), then the combined organics were dried over Na₂SO₄ and concentrated in vacuo. The crude material was purified by column chromatography over silica (120 g cartridge) eluting with a gradient of ethyl acetate (5% to 80%; v/v) in iso-hexane to afford the title compound (280 mg, 0.767 mmol, 23.07% yield) as a yellow oil. MS (ES+) m/z=351.1 [M+H]+. ¹H NMR (400 MHz, CDCl₃) δ 7.71 (d, J=1.0 Hz, 1H), 5.64-5.43 (m, 1H), 4.93-4.63 (m, 1H), 4.00-3.68 (m, 4H), 3.38 (s, 3H), 2.35 (d, J=0.9 Hz, 3H), 2.27-1.97 (m, 4H).

### 3-Methoxy-2-(4-methyl-3-nitro-pyrazol-1-yl)-1-[(2S)-2-(trifluoromethyl)pyrrolidin-1-yl]propan-1-one

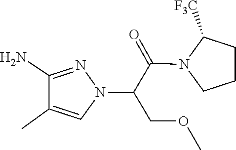 [SMILES: COCC(C(=O)N1CCC[C@H]1C(F)(F)F)n1cc(C)c(N)n1]

To a solution of cesium 3-methoxy-2-(4-methyl-3-nitro-pyrazol-1-yl)propanoate (1.20 g, 3.32 mmol), DIPEA (1.8 mL, 10.0 mmol) and HATU (1.50 g, 3.94 mmol) in DMF (20 mL) was added (2S)-2-(trifluoromethyl) pyrrolidine (650 mg, 4.67 mmol). The reaction was stirred at rt overnight, then it was diluted with EtOAc (100 mL) and washed with aq. LiCl 1M (150 mL). The aqueous solution was extracted with EtOAc (150 mL), then the combined organics were dried over Na₂SO₄ and concentrated in vacuo. The crude material was purified by column chromatography over silica (120 g cartridge) eluting with a gradient of ethyl acetate (5% to 80%; v/v) in iso-hexane to afford the title compound (280 mg, 0.767 mmol, 23.07% yield) as a yellow oil. MS (ES+) m/z=351.1 [M+H]+. ¹H NMR (400 MHz, CDCl₃) δ 7.71 (d, J=1.0 Hz, 1H), 5.63-5.43 (m, 1H), 4.89-4.68 (m, 1H), 4.03-3.69 (m, 4H), 3.39 (s, 3H), 2.35 (d, J=0.9 Hz, 3H), 2.28-1.95 (m, 4H).

### N-(2-morpholinoethyl)-2-(3-nitropyrazol-1-yl)acetamide

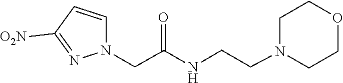 [SMILES: O=C(Cn1ccc([N+](=O)[O-])n1)NCCN1CCOCC1]

Prepared similarly using 4-(2-aminoethyl) morpholine (992 mg, 7.62 mmol, 1.5 eq) at 100° C. for 144 h to afford the title compound (750 mg, 2.65 mmol, 53%) as a yellow solid after normal phase chromatography (SiO₂) eluting with 0-100% EtOAc: PE followed by 0-20% MeOH: DCM. MS (ES+) m/z 284.3 (M+H).

### Nitropyrazole N-Alkylation Via Mitsunobu Reaction

### From Mitsunobu Reaction with 3-nitro-1H-pyrazole

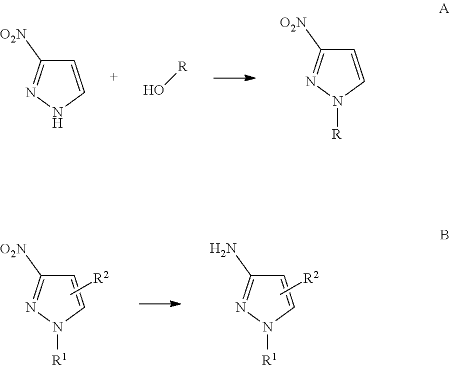

General Method A: Mitsunobu reaction with 3-nitro-1H-pyrazole DIAD (1.1-1.6 eq) was added dropwise to a solution of alcohol (1.0 eq), 3-nitro-1H-pyrazole (1.0 eq) and PPh₃ (1.1-1.5 eq) in anhydrous THF (0.2-0.4 M) with stirring, under nitrogen. The mixture was stirred overnight at RT before concentrating to dryness to give the crude product, which was chromatographed (SiO₂) eluting with 0-50% EtOAc: PE.

### tert-Butyl(3R)-3-(3-nitropyrazol-1-yl) pyrrolidine-1-carboxylate

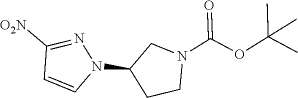 [SMILES: CC(C)(C)OC(=O)N1CC[C@@H](n2ccc([N+](=O)[O-])n2)C1]

Prepared as described in Method A from 3-nitro-1H-pyrazole (1.2 g, 12.7 mmol, 1.0 eq), (S)-1-N-boc-3-hydroxy-pyrrolidine (2.0 g, 10.7 mmol, 1.0 eq), PPh₃ (3.36 g, 12.82 mmol, 1.2 eq) and DIAD (2.52 mL, 12.82 mmol, 1.2 eq) in anhydrous THF (50 mL) to afford the title compound
(1.81 g, 6.41 mmol, 60%) as a clear colourless oil after normal phase chromatography (SiO₂) eluting with 0-50% EtOAc: PE. MS (ES+) m/z 227.2 (M-fBu+H). ¹H NMR (Chloroform -d, 300 MHz) δ 7.51 (s, 1H), 7.07 (d, 1H, J=2.1 Hz), 5.73-5.64 (m, 1H), 3.87-3.50 (m, 4H), 2.55-2.45 (m, 1H), 2.43-2.30 (m, 1H), 1.43 (s, 9H) ppm

The following examples were prepared in a similar manner from the appropriate alcohol.

| Structure | Name | Analytical Data |
|---|---|---|
| 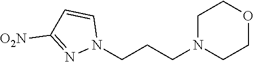 [SMILES: O=[N+]([O-])c1ccn(CCCN2CCOCC2)n1] | 4-[3-(3-nitropyrazol- 1-yl)propyl] morpholine | MS (ES+) m/z 241.3 (M + H). |
| 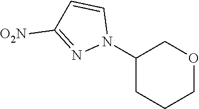 [SMILES: O=[N+]([O-])c1ccn(C2CCCOC2)n1] | 3-nitro-1-tetrahydro- pyran-3-yl-pyrazole | ¹H NMR (300 MHz, DMSO-d₆) δ 7.73 (d, J = 2.2 Hz, 1H), 7.26 (d, J = 2.2 Hz, 1H), 5.04 (tt, J = 10.1, 4.3 Hz, 1H), 4.04 (ddd, J = 10.6, 4.2, 1.9 Hz, 1H), 3.90- 3.81 (m, 1H), 3.60-3.49 (m, 1H), 3.44- 3.30 (m, 1H), 2.24-1.98 (m, 2H), 1.86- 1.61 (m, 2H). |
| 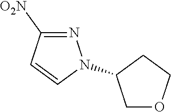 [SMILES: O=[N+]([O-])c1ccn([C@@H]2CCOC2)n1] | 3-nitro-1-[(3R)-tetra- hydrofuran-3-yl] pyrazole | ¹H NMR (300 MHz, DMSO-d₆) δ 7.71 (d, J = 2.2 Hz, 1H), 7.28 (d, J = 2.2 Hz, 1H), 5.69 (ddt, J = 7.8, 6.2, 3.2 Hz, 1H), 4.08-3.91 (m, 3H), 3.85 (td, J = 8.1, 5.0 Hz, 1H), 2.48-2.30 (m, 2H). |
| 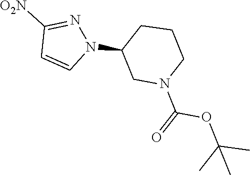 [SMILES: CC(C)(C)OC(=O)N1CCC[C@H](n2ccc([N+](=O)[O-])n2)C1] | tert-butyl (3S)-3-(3- nitropyrazol-1-yl)- piperidine-1-carbox- ylate | ¹H NMR (300 MHz, DMSO-d₆) δ 7.73 (d, J = 2.2 Hz, 1H), 7.28 (d, J = 2.2 Hz, 1H), 5.00-4.90 (m, 1H), 4.12 (d, J = 11.4 Hz, 1H), 3.79 (d, J = 13.2 Hz, 1H), 3.23 (s, 1H), 3.01-2.86 (m, 1H), 2.20-2.00 (m, 2H), 1.87 (dt, J = 13.2, 3.7 Hz, 1H), 1.55-1.44 (m, 1H), 1.37 (s, 9H). |
| 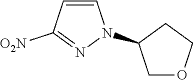 [SMILES: O=[N+]([O-])c1ccn([C@H]2CCOC2)n1] | 3-nitro-1-[(3S)-tetra- hydrofuran-3-yl] pyrazole | MS (ES+) m/z 184.2 (M + H). |
| 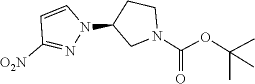 [SMILES: CC(C)(C)OC(=O)N1CC[C@H](n2ccc([N+](=O)[O-])n2)C1] | tert-butyl (3S)-3-(3- nitropyrazol-1-yl) pyrrolidine-1- carboxylate | ¹H NMR (300 MHz, DMSO-d₆) δ 7.73 (d, J = 2.2 Hz, 1H), 7.30 (d, J = 2.2 Hz, 1H), 5.66 (dt, J = 6.0, 3.1 Hz, 1H), 3.71 (qd, J = 13.8, 12.9, 4.7 Hz, 2H), 3.44 (q, J = 6.0, 5.4 Hz, 2H), 2.38 (dd, J = 15.8, 7.3 Hz, 2H), 1.40 (d, J = 4.3 Hz, 9H). |
| 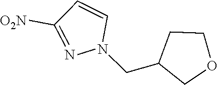 [SMILES: O=[N+]([O-])c1ccn(CC2CCOC2)n1] | 3-nitro-1-(tetra- hydrofuran-3-yl- methyl)pyrazole | MS (ES+) m/z 184.2 (M + H). |

### 2. Aromatic and Heteroaromatic Amine Precursors

### General Method B: Hydrogenation of N-Substituted Nitro-Pyrazoles

A mixture of 3-nitro-1-alkylpyrazoles (1.0 eq) and Pd/C(10% w/w, 0.05-0.15 eq) in MeOH(0.01-0.26 M) or THF was placed under a hydrogen atmosphere and stirred at RT for 12-72 h. The reaction was monitored by consumption of starting material via UPLC. The mixture was filtered through a pad of dicalite and concentrated in vacuo to afford the amine product.

### 3-(3-Aminopyrazol-1-yl)propan-1-ol

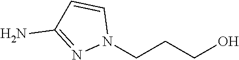

Prepared as described in Method B from 2-(3-nitropyrazol-1-yl) ethanol (500 mg, 3.18 mmol, 1.0 eq) and Pd/C(10% w/w, 17 mg, 0.159 mmol, 0.05 eq) in MeOH(50 mL) after 72 h to afford the title compound (2.1 g, 14.9 mmol, 93%) as a colourless oil. MS (ES+) m/z 141.9 (M+H). ¹H NMR (300 MHz, DMSO-d₆) δ 7.26 (d, J=2.2 Hz, 1H), 5.34 (d, J=2.2 Hz, 1H), 4.54-4.42 (m, 3H), 3.86 (t, J=6.9 Hz, 2H), 3.45-3.30 (m, 2H), 1.81 (p, J=6.6 Hz, 2H).

The following N-substituted amino-pyrazoles were obtained in a similar manner:

| Structure | Name | Analytical Data |
|---|---|---|
| 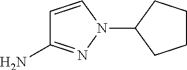 [SMILES: Nc1ccn(C2CCCC2)n1] | 1-cyclopentylpyrazol- 3-amine | MS (ES+) m/z 151.9 (M + H). |
| 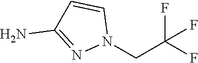 [SMILES: Nc1ccn(CC(F)(F)F)n1] | 1-(2,2,2-trifluoroethyl)- pyrazol-3-amine | MS (ES+) m/z 165.9 (M + H). |
| 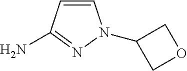 | 1-(oxetan-3-yl)pyrazol- 3-amine | ¹H NMR (300 MHz, DMSO-d₆) δ 7.41 (d, J = 2.1 Hz, 1H), 5.44 (d, J = 2.1 Hz, 1H), 5.27 (q, J = 6.8 Hz, 1H), 4.81 (p, J = 6.3 Hz, 6H). |
| 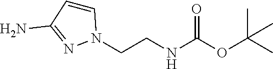 [SMILES: CC(C)(C)OC(=O)NCCn1ccc(N)n1] | tert-butyl N-[2-(3- aminopyrazol-1-yl) ethyl]carbamate | MS (ES+) m/z 227.3 (M + H). |
| 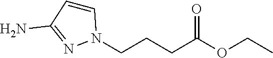 | ethyl 4-(3-aminopyr- azol-1-yl)butanoate | MS (ES+) m/z 197.9 (M + H). ¹H NMR (300 MHz, Chloro- form-d) δ 7.11 (d, J = 2.3 Hz, 1H), 5.56 (d, J = 2.3 Hz, 1H), |
| | | 4.12 (q, J = 7.2 Hz, 2H), 3.97 |
| | | (t, J = 6.6 Hz, 2H), 3.49 (s, |
| | | 2H), 2.28 (td, J = 7.1, 1.0 Hz, |
| | | 2H), 2.20-2.03 (m, 2H), 1.25 |
| | | (t, J = 7.1 Hz, 3H). |
| 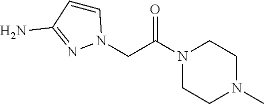 [SMILES: CN1CCN(C(=O)Cn2ccc(N)n2)CC1] | 2-(3-aminopyrazol-1- yl)-1-(4-methylpiper- azin-1-yl)ethenone | MS (ES+) m/z 223.9 (M + H). |
| 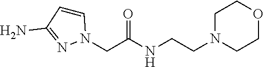 [SMILES: Nc1ccn(CC(=O)NCCN2CCOCC2)n1] | 2-(3-aminopyrazol-1- yl)-N-(2-morpholino- ethyl)acetamide | MS (ES+) m/z 254.3 (M + H). |
| 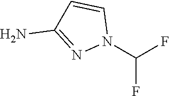 | 1-(difluoromethyl)pyr- azol-3-amine | MS (ES+) m/z 133.9 (M + H). |
| 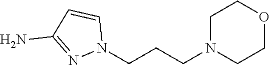 [SMILES: Nc1ccn(CCCN2CCOCC2)n1] | 1-(3-morpholinoprop- yl)pyrazol-3-amine) | MS (ES+) m/z 211.3 (M + H). |
| 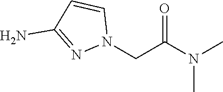 | 2-(3-aminopyrazol-1- yl)-N,N-dimethyl- acetamide | ¹H NMR (300 MHz, Chloro- form-d) δ 7.27 (d, J = 2.4 Hz, 1H), 5.68 (d, J = 2.4 Hz, 1H), 4.78 (s, 2H), 3.82 (s, 2H), 3.06 (s, 3H), 2.98 (s, 3H). |
| 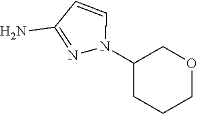 [SMILES: Nc1ccn(C2CCCOC2)n1] | 1-tetrahydropyran-3- ylpyrazol-3-amine | MS (ES+) m/z 168.3 (M + H). |
| 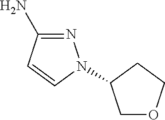 [SMILES: Nc1ccn([C@@H]2CCOC2)n1] | 1-[(3R)-tetrahydro- furan-3-yl]pyrazol-3- amine | MS (ES+) m/z 154.2 (M + H). |
| 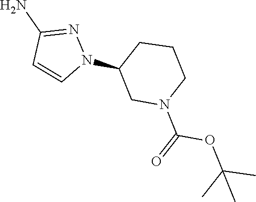 [SMILES: CC(C)(C)OC(=O)N1CCC[C@H](n2ccc(N)n2)C1] | tert-butyl (3S)-3-(3- aminopyrazol-1-yl) piperidine-1-carbox- ylate | MS (ES+) m/z 267.3 (M + H). |
| 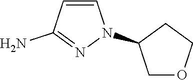 [SMILES: Nc1ccn([C@H]2CCOC2)n1] | 1-[(3S)-tetrahydro- furan-3-yl]pyrazol-3- amine | MS (ES+) m/z 154.3 (M + H). |
| 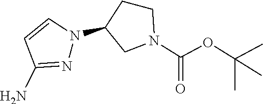 [SMILES: CC(C)(C)OC(=O)N1CC[C@H](n2ccc(N)n2)C1] | tert-butyl (3S)-3-(3- aminopyrazol-1-yl) pyrrolidine-1-carbox- ylate | MS (ES-) m/z 251.1 (M − H). |
| 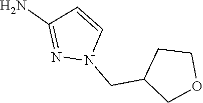 [SMILES: Nc1ccn(CC2CCOC2)n1] | 1-(tetrahydrofuran-3- ylmethyl)pyrazol-3- amine | MS (ES+) m/z 168.3 (M + H). |
| 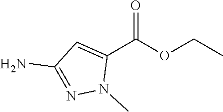 | ethyl 5-amino-2-meth- yl-pyrazole-3-carbox- ylate | MS (ES+) 169.9 (M + H). |
| 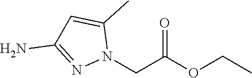 | ethyl 2-(3-amino-5- methyl-pyrazol-1-yl)- acetate | MS (ES+) m/z 184.2 (M + H). ¹H NMR (300 MHz, DMSO-d₆) δ 5.26 (d, J = 0.8 Hz, 1H), 4.64 (s, 2H), 4.48 (s, 2H), 4.12 (q, J = 7.1 Hz, 2H), 2.06 (d, J = 0.7 |
| | | Hz, 3H), 1.20 (t, J = 7.1 Hz, 3H) |
| 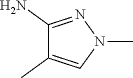 | 1,4-dimethylpyrazol- 3-amine | MS (ES+) m/z 112.1 (M + H). ¹H NMR (300 MHz, DMSO-d₆) δ 7.05 (s, 1H), 4.31 (s, 2H), 3.50 (s, 3H), 1.79 (s, 3H). |
| 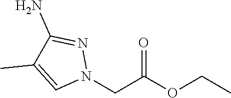 | ethyl 2-(3-amino-4- methyl-pyrazol-1-yl) acetate | MS (ES+) m/z 184.2 (M + H). ¹H NMR (300 MHz, DMSO- d₆) δ 7.15 (d, J = 0.9 Hz, 1H), 4.66 (s, 2H), 4.10 (q, J = 7.1 Hz, 2H), 1.82 (d, J = 0.7 Hz, 3H), 1.19 (t, J = 7.1 Hz, 3H). |
| 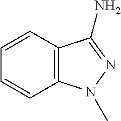 | 1-methylindazol-3- amine | MS (ES+) m/z 148.1 (M + H). ¹H NMR (300 MHz, DMSO- d₆) δ 7.68 (dt, J = 8.0, 1.0 Hz, 1H), 7.33-7.23 (m, 2H), 6.90 (ddd, J = 7.9, 6.3, 1.4 Hz, 1H), 5.51-5.31 (brs, 2H), 3.72 (s, 3H). |
| 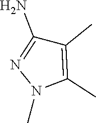 | 1,4,5-trimethylpyr- azol-3-amine | MS (ES+) m/z 126.0 (M + H). ¹H NMR (300 MHz, Chloro- form-d) δ 3.63 (s, 2H), 3.56 (s, 3H), 2.06 (s, 3H), 1.81 (s, 3H). |
| 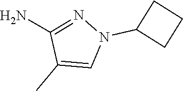 | 1-cyclobutyl-4-meth- yl-1H-pyrazol-3- amine | ¹H NMR (300 MHz, DMSO) δ 7.16 (d, J = 0.9 Hz, 1H), 4.51- 4.41 (m, 1H), 4.40 (s, 2H), 2.42-2.28 (m, 2H), 2.27-2.17 (m, 2H), 1.79 (d, J = 0.8 Hz, 3H), 1.73-1.60 (m, 2H). MS |
| | | (ES+) m/z 152.2 (M + H)⁺ |
| 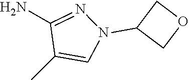 | 4-methyl-1-(oxetan- 3-yl)-1H-pyrazol-3- amine | ¹H NMR (300 MHz, MeOD) δ 7.27 (d, J = 0.9 Hz, 1H), 5.34- 5.19 (m, 1H), 4.96 (d, J = 6.9 Hz, 4H), 1.94 (d, J = 0.8 Hz, 3H). MS (ES+) m/z 154.2 (M + H)⁺ |
| 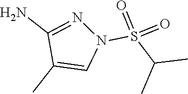 | 1-(isopropylsulfonyl)- 4-methyl-1H-pyrazol- 3-amine | ¹H NMR (300 MHz, DMSO) δ 7.66 (d, J = 1.2 Hz, 1H), 5.50 (s, 2H), 3.57 (hept, J = 6.8 Hz, 1H), 1.88 (d, J = 1.1 Hz, 3H), 1.15 (d, J = 6.8 Hz, 6H). MS (ES+) m/z 204.2 (M + H)⁺ |
| 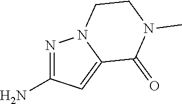 | 2-amino-5-methyl-6,7- dihydropyrazolo[1,5- a]pyrazin-4(5H)-one | 1H NMR (300 MHz, DMSO- d6) δ 5.82 (s, 1H), 4.84 (s, 2H), 4.11-4.01 (m, 2H), 3.74-3.63 (m, 2H), 2.96 (s, 3H). |
| 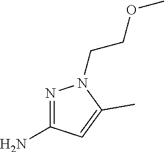 | 1-(2-methoxyethyl)-5- methyl-pyrazol-3- amine | ¹H NMR (300 MHz, DMSO- d6) δ 5.19 (d, J = 0.8 Hz, 1H), 4.39 (s, 2H), 3.88 (t, J = 5.6 Hz, 2H), 3.55 (t, J = 5.6 Hz, 2H), 3.21 (s, 3H), 2.10 (d, J = 0.7 Hz, 3H). MS (ES+) m/z 156.2 (M + H)⁺. |
| 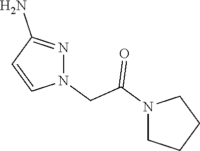 [SMILES: Nc1ccn(CC(=O)N2CCCC2)n1] | 2-(3-aminopyrazol-1- yl)-1-pyrrolidin-1-yl- ethanone | MS (ES+) m/z = 195 [M + H]+; ¹H NMR (400 MHz, CHLORO- FORM-D) δ 7.27 (d, J = 2.4 Hz, 1H), 5.65 (d, J = 2.3 Hz, 1H), 4.67 (s, 2H), 3.62 (brs, 2H), 3.47 (dt, J = 15.4, 6.9 Hz, 4H), 2.03-1.91 (m, 2H), 1.85 (pd, J = 6.8, 0.9 Hz, 2H). |
| 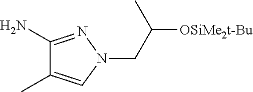 | 1-(2-((tert-butyldimeth- ylsilyl)oxy)propyl)-4- methyl-1H-pyrazol-3- amine | MS (ES+) m/z = 270.3 [M + H]+. 1H NMR (400 MHz, CHLOROFORM-D) δ 6.94 (d, J = 0.8 Hz, 1H), 4.09 (dqd, J = 7.7, 6.1, 4.1 Hz, 1H), 3.81 (dd, J = 13.8, 4.0 Hz, 1H), 3.74-3.64 |
| | | (m, 1H), 3.19 (s, 2H), 1.90 (d, |
| | | J = 0.9 Hz, 3H), 1.11 (d, J = 6.2 |
| | | Hz, 3H), 0.83 (s, 9H), −0.04 (s, |
| | | 3H), −0.16 (s, 3H). |
| 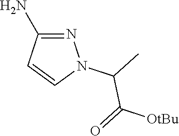 | tert-butyl 2-(3-amino- 1H-pyrazol-1-yl)pro- panoate | MS (ES+) m/z = 212.1 [M + H]+, ¹H NMR (400 MHz, DMSO-D6) δ 7.36 (d, J = 2.3 Hz, 1H), 5.41 (d, J = 2.3 Hz, 1H), 4.70 (q, J = 7.3 Hz, 1H), 4.60 (s, 2H), 1.52 (d, J = 7.3 Hz, 3H), 1.36 (s, 9H). |
| 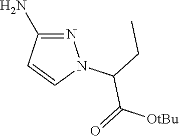 [SMILES: CCC(C(=O)OC(C)(C)C)n1ccc(N)n1] | tert-butyl 2-(3-amino- 1H-pyrazol-1-yl) butanoate | MS (ES+) m/z = 226.2 [M + H]+. ¹H NMR (400 MHz, DMSO-D6) δ 7.36 (d, J = 2.3 Hz, 1H), 5.42 (d, J = 2.3 Hz, 1H), 4.66 (d, J = 21.8 Hz, 2H), 4.47 (t, J = 7.7 Hz, 1H), 2.01- 1.88 (m, 2H), 1.37 (s, 9H), 0.79 (t, J = 7.4 Hz, 3H). |
| 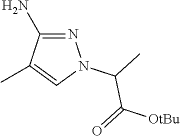 [SMILES: Cc1cn(C(C)C(=O)OC(C)(C)C)nc1N] | tert-butyl 2-(3-amino- 4-methyl-1H-pyrazol- 1-yl)propanoate | MS (ES+) m/z = 226.3 [M + H]+, 1H NMR (400 MHz, CHLOROFORM-D) δ 7.09 (q, J = 0.8 Hz, 1H), 4.67 (q, J = 7.4 Hz, 1H), 3.50 (s, 2H), 1.93 (d, J = 0.8 Hz, 3H), 1.64 (d, J = 7.3 Hz, 3H), 1.43 (s, 9H). |
| 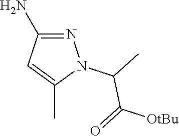 [SMILES: Cc1cc(N)nn1C(C)C(=O)OC(C)(C)C] | tert-butyl 2-(3-amino- 5-methyl-1H-pyrazol- 1-yl)propanoate | MS (ES+), m/z = 226.2 [M + H]+; 1H NMR (400 MHz, CHLOROFORM-D) δ 7.09 (q, J = 0.8 Hz, 1H), 4.67 (q, J = 7.4 Hz, 1H), 3.50 (s, 2H), 1.93 (d, J = 0.8 Hz, 3H), 1.64 (d, J = 7.3 Hz, 3H), 1.43 (s, 9H). |
| 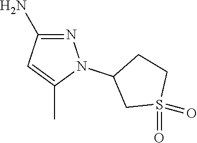 | 3-(3-amino-5-methyl- 1H-pyrazol-1-yl) tetrahydrothiophene 1,1-dioxide | 1H NMR (400 MHz, DMSO- D6) δ 5.24 (t, J = 0.7 Hz, 1H), 5.03-4.88 (m, 1H), 4.64 (s, 2H), 3.64-3.52 (m, 1H), 3.50-3.37 (m, 1H), 3.26-3.11 (m, 2H), 2.44-2.33 (m, 2H), 2.14 (d, J = 0.8 Hz, 3H). |
| 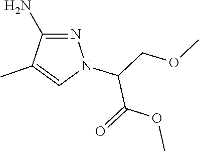 | methyl 2-(3-amino-4- methyl-1H-pyrazol-1- yl)-3-methoxypropan- oate | MS (ES+), m/z = 214.2 [M + H]+. 1H NMR (400 MHz, CDCl3) δ 7.19 (q, J = 0.9 Hz, 1H), 4.89-4.82 (m, 1H), 3.99 (ddd, J = 10.1, 6.6, 1.1 Hz, 1H), 3.91 (ddd, J = 10.0, 4.4, 1.0 Hz, 1H), 3.74 (s, 3H), 3.35 (s, 3H), 1.92 (d, J = 0.9 Hz, 3H). |
| 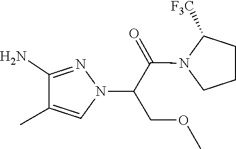 | 2-(3-amino-4-methyl- 1H-pyrazol-1-yl)-3- methoxy-1-((S)-2- (trifluoromethyl)pyr- rolidin-1-yl)propan- 1-one | MS (ES+) m/z = 351.1 [M + H]+; ¹H NMR (400 MHz, CDCl3) δ 7.71 (d, J = 1.0 Hz, 1H), 5.63-5.43 (m, 1H), 4.89- 4.68 (m, 1H), 4.03-3.69 (m, 4H), 3.39 (s, 3H), 2.35 (d, J = 0.9 Hz, 3H), 2.28-1.95 (m, 4H). ¹⁹F NMR (376 MHz, CDCl3) δ −73.97 (d, J = 7.6 Hz), −74.45 |
| | | (d, J = 7.6 Hz). Approx. 6:1 |
| | | mixture of diastereoisomers. |
| 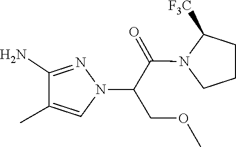 | 2-(3-amino-4-methyl- 1H-pyrazol-1-yl)-3- methoxy-1-((R)-2- (trifluoromethyl)pyr- rolidin-1-yl)propan-1- one | MS (ES+) m/z = 351.1 [M + H]+; ¹H NMR (400 MHz, CDCl3) δ 7.71 (d, J = 1.0 Hz, 1H), 5.64-5.43 (m, 1H), 4.93- 4.63 (m, 1H), 4.00-3.68 (m, 4H), 3.38 (s, 3H), 2.35 (d, J = 0.9 Hz, 3H), 2.27-1.97 (m, 4H). ¹⁹F NMR (376 MHz, CDCl₃) δ −73.97 (d, J = 7.5 Hz), −74.45 |
| | | (d, J = 7.6 Hz). Approx. 6:1 |
| | | mixture of diastereoisomers. |

### tert-Butyl(3R)-3-(3-aminopyrazol-1-yl) pyrrolidine-1-carboxylate

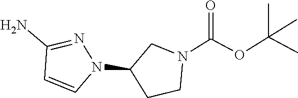 [SMILES: CC(C)(C)OC(=O)N1CC[C@@H](n2ccc(N)n2)C1]

Prepared by Method B from tert-butyl(3R)-3-(3-nitropyrazol-1-yl) pyrrolidine-1-carboxylate
(400 mg, 1.41 mmol) to afford the title compound (349 mg, 1.38 mmol, 98% yield) as a yellow solid after normal phase chromatography (SiO₂) eluting with 0-20% MeOH: DCM MS (ES+) 253.3 (M+H). ¹H NMR (Chloroform -d, 300 MHz) δ 7.29 (s, 1H), 5.57 (d, 1H, J=1.8 Hz), 4.76-4.66 (m, 1H), 3.79-3.60 (m, 3H), 3.51 (br s, 2H), 3.49-3.40 (m, 1H), 2.55-2.39 (m, 1H), 2.24 (dq, 1H, J=12.5, 7.0 Hz), 1.45 (s, 9H) ppm.

### 2-(3-Amino-4-ethyl-pyrazol-1-yl)acetonitrile

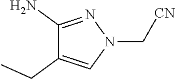

A solution of 2-(3-nitro-4-vinyl-pyrazol-1-yl)acetonitrile (67 mg, 0.342 mmol) in a mixture of ethanol (2 mL) and AcOEt (2 mL) was degassed twice with N₂. Palladium on activated Carbon (35 mg, 0.0329 mmol) was slurried in water (0.5 mL) and added to the reaction, then the solution was degassed twice with H₂. The reaction was stirred under 1 atm of H2 overnight. The reaction mixture was filtered through a short pad of Celite. The filtrate was concentrated in vacuo, to afford the title compound (51 mg, 0.183 mmol, 53.56% yield) as a yellow film. MS (ES+) m/z=151.0 [M+H]+. ¹H NMR (400 MHz, DMSO-d₆) δ 7.21 (s, 1H), 5.06 (s, 2H), 4.67 (s, 2H), 2.22 (q, J=7.7 Hz, 2H), 1.06 (t, J=7.5 Hz, 3H).

### 1-(2,2-Difluoroethyl)-4-ethyl-1H-pyrazol-3-amine

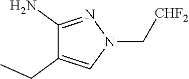

A solution of 1-(2,2-difluoroethyl)-3-nitro-4-vinyl-pyrazole (125 mg, 0.493 mmol) in a mixture of ethanol (2.5 mL) and AcOEt (2.5 mL) was degassed twice with N₂. Palladium on activated Carbon (53 mg, 0.0498 mmol) was slurried in water (0.5 mL) and added to the reaction, then the solution was degassed twice with H₂. The reaction was stirred under 1 atm of H2 overnight then was filtered through a short pad of Celite, and concentrated in vacuo, affording 1-(2,2-difluoroethyl)-4-ethyl-pyrazol-3-amine (87 mg, 0.400 mmol, 81.06% yield) as a colourless oil.MS (ES+) m/z=176.1 [M+H]+. ¹H NMR (400 MHz, DMSO-d₆) δ 7.17 (s, 1H), 6.17 (tt, J=55.5, 4.1 Hz, 1H), 4.47 (s, 2H), 4.22 (td, J=14.8, 4.1 Hz, 2H), 2.22 (q, J=7.5 Hz, 2H), 1.06 (t, J=7.5 Hz, 3H). ¹⁹F NMR (376 MHz, DMSO-d₆) 0-122.15 (dt, J=55.5, 14.8 Hz).

### 4-Ethyl-1-(2-fluoroethyl)-1H-pyrazol-3-amine

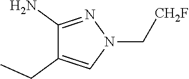

A solution of 1-(2-fluoroethyl)-3-nitro-4-vinyl-pyrazole (111 mg, 0.535 mmol) in a mixture of ethanol (2.5 mL) and AcOEt (2.5 mL) was degassed twice with N₂. Palladium on activated Carbon
(55 mg, 0.0517 mmol) was slurried in water (0.5 mL) and added to the reaction, then the solution was degassed twice with H₂. The reaction was stirred under 1 atm of H2 overnight then was filtered through a short pad of Celite, and concentrated in vacuo, to afford the title compound (82 mg, 0.464 mmol, 86.68% yield) as a colourless oil. MS (ES+) m/z=158.1 [M+H]⁺, ¹H NMR (400 MHz, DMSO-d₆) δ 7.14 (s, 1H), 4.62 (dt, J=47.4, 4.8 Hz, 2H), 4.38 (s, 2H), 4.06 (dt, J=27.4, 4.8 Hz, 2H), 2.21 (q, J=7.5 Hz, 2H), 1.06 (t, J=7.5 Hz, 3H).

### 4-Ethyl-1-(4-methoxybenzyl)-1H-pyrazol-3-amine

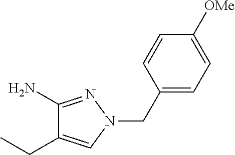 [SMILES: CCc1cn(Cc2ccc(OC)cc2)nc1N]

A solution of 1-[(4-methoxyphenyl)methyl]-3-nitro-4-vinyl-pyrazole (130 mg, 0.431 mmol) in a mixture of ethanol (2 mL) and AcOEt (2 mL) was degassed N2, palladium on activated carbon
(50 mg, 0.0470 mmol) was slurried in water (0.5 mL) and added to the reaction, then the solution was degassed with H₂. The reaction was stirred under 1 atm of H2 overnight, the reaction mixture was filtered through Celite and the filtrate was concentrated in vacuoto afford the title compound
(91 mg, 0.392 mmol, 90.83% yield) as a colourless oil. MS (ES+) m/z=232.2 [M+H]⁺, 79% purity. ¹H NMR (400 MHz, DMSO-d₆) δ 7.18 (s, 1H), 7.13 (d, J=8.7 Hz, 2H), 6.86 (d, J=8.7 Hz, 2H), 4.87 (s, 2H), 4.33 (s, 2H), 3.71 (s, 3H), 2.20 (q, J=7.5 Hz, 2H), 1.05 (t, J=7.5 Hz, 3H).

### 4-Fluoro-1-(4-methoxybenzyl)-1H-pyrazol-3-amine

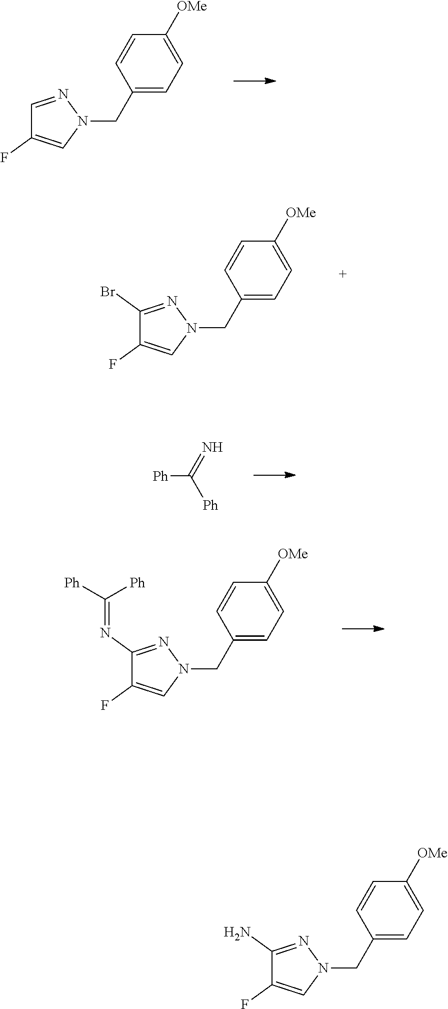 [SMILES: COc1ccc(Cn2cc(F)cn2)cc1 ; COc1ccc(Cn2cc(F)c(Br)n2)cc1 ; N=C(c1ccccc1)c1ccccc1 ; COc1ccc(Cn2cc(F)c(N=C(c3ccccc3)c3ccccc3)n2)cc1 ; COc1ccc(Cn2cc(F)c(N)n2)cc1]

To a stirring solution of 4-fluoro-1H-pyrazole (2.00 g, 23.2 mmol) in NMP (30 mL) at 0° C. under inert atmosphere was added sodium hydride, 60% in mineral oil (1.20 g, 30.0 mmol) in small portions. The mixture was stirred for 1 h, then 4-methoxybenzyl chloride (3.5 mL, 25.8 mmol) was added. The reaction was allowed to rt and stirred overnight, then was poured into ice (100 g) and extracted with DCM (3×100 mL). The combined organics were dried over Na₂SO₄ and concentrated. The crude material was purified by column chromatography over silica (120 g cartridge) eluting with a gradient of DCM (0% to 70%; v/v) in iso-hexane to afford the 4-fluoro-1-[(4-methoxyphenyl)methyl]pyrazole (4.33 g, 20.2 mmol, 86.83% yield) as a colourless oil. MS (ES+) m/z=207.2 [M+H]+. ¹H NMR (400 MHz, CDCl₃) δ 7.91 (dd, J=4.7, 0.9 Hz, 1H), 7.45 (dd, J=4.3, 0.9 Hz, 1H), 7.20 (d, J=8.8 Hz, 2H), 6.90 (d, J=8.8 Hz, 2H), 5.14 (s, 2H), 3.72 (s, 3H). ¹⁹F NMR
(376 MHz, CDCl₃) δ-177.74 (t, J=4.6 Hz).

To a stirred solution of 4-fluoro-1-[(4-methoxyphenyl)methyl]pyrazole (4.33 g, 19.9 mmol) in anhydrous THF (60 mL) at −78° C. was added n-butyllithium, 2.5 M in hexanes (11 mL, 26.3 mmol) dropwise. The reaction mixture was stirred at −78° C. for 30 min under nitrogen atmosphere, then a solution of 1,2-Dibromotetrachloroethane (9.10 g, 27.9 mmol) in anhydrous THF (20 mL) was added dropwise. The resulting solution was allowed to warm to room temperature and stirred at room temperature for 2 h. The reaction mixture was cooled to 0° C. in an ice bath and quenched with sat. aq. NH₄Cl (80 mL) and water (40 mL). The mixture was extracted with EtOAc (3×80 mL). The combined organics were washed with brine (160 mL), dried over Na₂SO₄, filtered and concentrated. The crude material was purified by column chromatography over silica (220 g cartridge) eluting with a gradient of ethyl acetate (0% to 20%; v/v) in iso-hexane to afford 3-bromo-4-fluoro-1-[(4-methoxyphenyl)methyl]pyrazole (4.17 g, 9.95 mmol, 49.88% yield). MS (ES-) m/z=283.2 [M-H]−. ¹H NMR (400 MHz, CDCl₃) δ 7.42 (d, J=4.2 Hz, 1H), 7.22-7.16 (m, 2H), 6.89-6.83 (m, 2H), 5.22 (s, 2H), 3.79 (s, 3H). ¹⁹F NMR (376 MHz, CDCl₃) δ-171.62 (d, J=4.8 Hz). 3-bromo-4-fluoro-1-[(4-methoxyphenyl)methyl]pyrazole (4.17 g, 14.6 mmol) and Xantphos (1.70 g, 2.94 mmol) were dissolved in 1,4-dioxane (50 mL), benzophenoneimine (3.8 mL, 22.6 mmol) and caesium carbonate (14.30 g, 43.9 mmol) were added, the mixture was degassed with nitrogen for 5 minutes, palladium (II)acetate (840 mg, 3.74 mmol) added, the mixture degassed with nitrogen for 5 more minutes, then the reaction was stirred at 110° C. overnight. The mixture was filtered through celite, rinsed with DCM, and the filtrate was evaporated. The crude material was purified by column chromatography over silica (120 g cartridge) eluting with a gradient of ethyl acetate (0% to 40%; v/v) in iso-hexane to afford N-[4-fluoro-1-[(4-methoxyphenyl)methyl]pyrazol-3-yl]-1, 1-diphenyl-methanimine (6.30 g, 13.6 mmol, 92.81% yield) as a thick brown oil. MS (ES+) m/z=386.2 [M+H]⁺, ¹H NMR (400 MHz, CDCl₃) δ 7.95 (dd, J=8.4, 1.2 Hz, 1H), 7.76 (dd, J=8.4, 1.3 Hz, 2H), 7.60 (t, J=7.8 Hz, 1H), 7.55-7.49 (m, 1H), 7.47-7.37 (m, 2H), 7.39-7.33 (m, 1H), 7.31-7.27 (m, 1H), 7.27-7.23 (m, 1H), 7.08 (d, J=4.2 Hz, 1H), 6.91 (dd, J=8.3, 1.3 Hz, 2H), 6.86-6.81 (m, 2H), 5.27 (s, 2H), 3.77 (s, 3H).

N-[4-fluoro-1-[(4-methoxyphenyl)methyl]pyrazol-3-yl]-1,1-diphenyl-methanimine (1.00 g, 2.28 mmol) was dissolved in a mixture of AcOEt (10 mL) and EtOH(10 mL), palladium on activated Carbon (250 mg, 0.235 mmol) suspended in water (0.5 mL) was added and the reaction was stirred at rt under 1 atm of H2 for 16 h. More Palladium on activated Carbon (250 mg, 0.235 mmol) suspended in water (0.5 mL) was added to the reaction mixture, which was stirred at rt under 1 atm of H2 for 4 days. The reaction mixture was filtered through Celite, evaporated, re-dissolved in a mixture of Ethanol (20 mL) and Ethyl acetate (5 mL), and acidified with Hydrochloric acid, 4N in dioxane (1.7 mL, 6.80 mmol). Palladium on activated Carbon (500 mg, 0.470 mmol) was suspended in water (1 mL) and added to the reaction mixture which was stirred under 1 atm of H2 for 2 days at rt, and at 80° C. for 3 further days. The reaction mixture was filtered through Celite, washed with ethanol, concentrated and loaded onto an SCX-2 cartridge (10 g) eluted with 7 M ammonia in MeOH(40 mL). The eluent was concentrated to dryness under reduced pressure to afford the title compound (278 mg, 1.25 mmol, 54.94% yield) as a brown oil. 1H-NMR analysis (sample reference: AC-0470-127-S9): ¹H NMR (400 MHz, DMSO-d₆) δ 7.19 (d, J=4.4 Hz, 1H), 7.11 (d, J=8.8 Hz, 2H), 6.86 (d, J=8.8 Hz, 2H), 5.17 (s, 2H), 5.00 (s, 2H), 3.71 (s, 3H). ¹⁹F NMR
(376 MHz, DMSO-d₆) δ-188.40 (d, J=4.4 Hz).

### General Procedure B2: Synthesis of Aminopyrazole Intermediates by Alkylation of Nitro-Pyrazoles and Hydrogenation

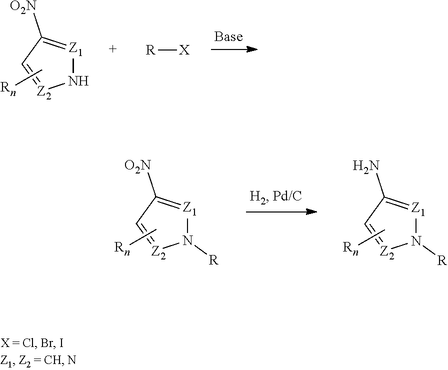

Potassium carbonate (1.5 equiv.) was added to a solution of alkyl halide (1 equiv.) and nitropyrazole (1 equiv.) in anhydrous THF or MeCN with stirring at room temperature under nitrogen. The reaction mixture was then heated to 75° C. with stirring for 4 hours, before cooling to room temperature and separating between ethyl acetate and water. The organic phase was washed with brine, dried over anhydrous magnesium sulfate, filtered and concentrated to give the crude product, which was purified using flash column chromatography over silica.

A solution of alkylated nitropyrazole (1 equiv.) in ethanol (0.1 M) was bubbled with nitrogen for 10 minutes before adding 10% Palladium on Carbon (10 mol %). The flask was then placed under vacuum and filled with nitrogen (x 2) before fitting a hydrogen balloon and stirring overnight at room temperature. The balloon was then removed and the reaction mixture bubbled with nitrogen for 5 minutes before filtering through dicalite, washing with ethyl acetate. The filtrate was concentrated to give the desired alkylated aminopyrazole.

The following heteroaromatic amines were obtained using general Method B2:

| Name | Structure | Analytical Data |
|---|---|---|
| 3-(3-amino-1H-pyr- azol-1-yl)-1,1,1- trifluoropropan-2-ol | 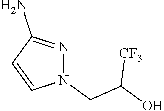 [SMILES: Nc1ccn(CC(O)C(F)(F)F)n1] | MS (ES+) 196.1 (M + H); ¹H NMR (DMSO- d₆, 300 MHz) δ 7.33 (d, 1H, J = 2.2 Hz), 6.55 (d, 1H, J = 6.7 Hz), 5.38 (d, 1H, J = 2.3 Hz), 4.62 (s, 2H), 4.32-4.24 (m, 1H), 4.08 (dd, 1H, J = 14.0, 3.4 Hz), 3.93 (dd, 1H, J = 14.0, 8.6 Hz) ppm; ¹⁹F NMR (DMSO-d₆, 282 MHz) δ −77.15 ppm |
| 1-(3,3,3-trifluoro-2- propoxypropyl)-1H- pyrazol-3-amine | 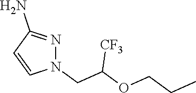 [SMILES: CCCOC(Cn1ccc(N)n1)C(F)(F)F] | MS (ES+) 238.2 (M + H); ¹H NMR (CDCl₃, 300 MHz) δ 7.17 (d, 1H, J = 2.3 Hz), 5.57 (d, 1H, J = 2.3 Hz), 4.22 (dd, 1H, J = 13.8, 2.5 Hz), 4.09-3.98 (m, 1H), 3.90 (dd, 1H, J = 13.8, 9.4 Hz), 3.67 (br s, 2H), 3.54 (dt, 1H, J = 8.9, 6.5 Hz), 3.16 (dt, 1H, J = 9.0, 6.6 Hz), 1.43 (h, |
| | | 2H, J = 7.3 Hz), 0.79 (t, 3H, J = 7.4 Hz) ppm; |
| | | ¹⁹F NMR (CDCl₃, 282 MHz) δ −76.57 (d, J = |
| | | 6.5 Hz) ppm |
| 2-(3-amino-1H- pyrazol-1-yl)-1- cyclopropylethan- 1-one | 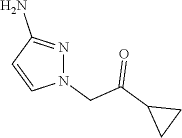 [SMILES: Nc1ccn(CC(=O)C2CC2)n1] | MS (ES+) 166.2 (M + H); ¹H NMR (CDCl₃, 300 MHz) 7.17 (d, 1H, J = 2.3 Hz), 5.66 (d, 1H, J = 2.3 Hz), 4.77 (s, 2H), 3.69 (br s, 2H), 1.77 (tt, 1H, J = 7.8, 4.6 Hz), 1.10-1.05 (m, 2H), 0.93-0.87 (m, 2H) ppm |
| tert-butyl 4-(2-(3- amino-1H-pyrazol- 1-yl)acetyl)piper- idine-1-carboxylate | 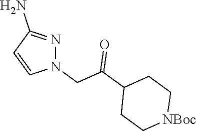 [SMILES: CC(C)(C)OC(=O)N1CCC(C(=O)Cn2ccc(N)n2)CC1] | MS (ES+) 309.1 (M + H); ¹H NMR (CDCl₃, 300 MHz) δ 7.12 (d, 1H, J = 2.4 Hz), 5.64 (d, 1H, J = 2.3 Hz), 4.71 (s, 2H), 4.10-4.03 (m, 2H), 3.73-3.60 (m, 2H), 2.71 (t, 2H, J = 11.8 Hz), 2.50 (tt, 1H, J = 11.3, 3.7 Hz), 1.77-1.71 (m, 2H), 1.58-1.48 (m, 2H), 1.41 (s, 9H) ppm |
| 2-(3-amino-1H- pyrazol-1-yl)-1- (tetrahydro-2H- pyran-4-yl)ethan- 1-one | 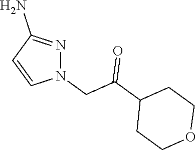 [SMILES: Nc1ccn(CC(=O)C2CCOCC2)n1] | MS (ES+) 210.5 (M + H); ¹H NMR (DMSO- d₆, 300 MHz) δ 7.31 (d, 1H, J = 2.3 Hz), 5.48 (d, 1H, J = 2.3 Hz), 4.86 (s, 2H), 3.84 (ddd, 2H, J = 11.4, 4.4, 2.6 Hz), 3.29 (td, 2H, J = 11.6, 2.4 Hz), 2.65 (tt, 1H, J = 11.4, 3.9 Hz), 1.71-1.65 (m, 2H), 1.53-1.39 (m, 2H) ppm |
| 1-(3-amino-1H- pyrazol-1-yl)butan- 2-one | 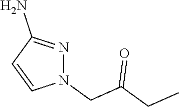 | MS (ES+) 154.1 (M + H); |
| 2-(3-amino-1H- pyrazol-1-yl)-1- cyclohexylethan- 1-one | 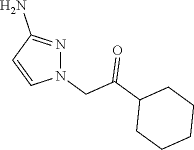 [SMILES: Nc1ccn(CC(=O)C2CCCCC2)n1] | MS (ES+) 208.6 (M + H); ¹H NMR (DMSO- d₆, 300 MHz) δ 7.27 (d, 1H, J = 2.3 Hz), 5.42 (d, 1H, J = 2.3 Hz), 4.55 (s, 2H), 2.40-2.31 (m, 1H), 1.79-1.58 (m, 6H), 1.24-1.15 (m, 4H) ppm |
| tert-butyl 4-(2-(3- amino-1H-pyrazol-1- yl)ethyl)piperazine- 1-carboxylate | 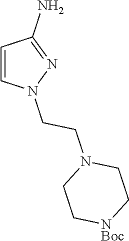 [SMILES: CC(C)(C)OC(=O)N1CCN(CCn2ccc(N)n2)CC1] | MS (ES+) 296.3 (M + H); |
| 2-(3-amino-4-meth- yl-1H-pyrazol-1-yl)- 1-(tetrahydro-2H- pyran-4-yl)ethan-1- one | 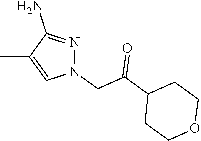 [SMILES: Cc1cn(CC(=O)C2CCOCC2)nc1N] | MS (ES+) 224.6 (M + H); ¹H NMR (DMSO- d₆, 300 MHz) δ 7.09 (s, 1H), 4.39 (s, 2H), 3.84 (ddd, 2H, J = 11.4, 4.4, 2.5 Hz), 3.33- 3.24 (m, 2H), 2.63 (tt, 1H, J = 11.4, 3.9 Hz), 1.81 (s, 3H), 1.70-1.64 (m, 2H), 1.52-1.38 (m, 2H) ppm |
| 2-(3-aminopyrazol- 1-yl)-1-pyrrolidin-1- yl-ethanone | 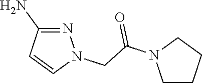 [SMILES: Nc1ccn(CC(=O)N2CCCC2)n1] | MS (ES+) m/z = 195 [M + H]+ 1H NMR (400 MHz, CHLOROFORM-D) δ 7.27 (d, J = 2.4 Hz, 1H), 5.65 (d, J = 2.3 Hz, 1H), 4.67 (s, 2H), 3.62 (brs, 2H), 3.47 (dt, J = 15.4, 6.9 Hz, 4H), 2.03-1.91 (m, 2H), 1.85 (pd, J = 6.8, 0.9 Hz, 2H) |
| 2-(3-amino-4-methyl- pyrazol-1-yl)-1- pyrrolidin-1-yl- ethanone | 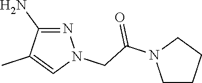 | MS (ES+) m/z = 209.1 [M + H]+. ¹H NMR (400 MHz, DMSO-D6) δ 7.07 (d, J = 0.9 Hz, 1H), 4.58 (s, 2H), 3.40 (t, J = 6.8 Hz, 3H), 3.27 (t, J = 6.9 Hz, 3H), 1.91-1.82 (m, 3H), 1.80 (d, J = 0.9 Hz, 4H), 1.79-1.71 (m, 3H). |
| 2-(3-amino-4-meth- yl-1H-pyrazol-1- yl)-N,N-diethylacet- amide | 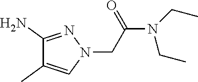 | ¹H NMR (400 MHz, DMSO-D6) δ 7.07-7.00 (m, 1H), 4.60 (s, 2H), 4.31 (s, 2H), 3.29 (q, J = 7.0 Hz, 2H), 3.20 (q, J = 7.1 Hz, 2H), 1.76 (d, J = 0.8 Hz, 3H), 1.12-1.02 (m, 3H), 0.97 (t, J = 7.1 Hz, 3H). |
| 5-methyl-1-(2-tetra- hydro pyran-2-yl- oxyethyl) pyrazol- 3-amine | 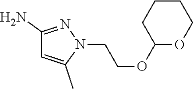 [SMILES: Cc1cc(N)nn1CCOC1CCCCO1] | ¹H NMR (400 MHz, DMSO-D6) δ 5.18 (d, J = 0.8 Hz, 1H), 4.46 (dd, J = 3.9, 2.8 Hz, 1H), 4.40 (s, 2H), 3.90 (t, J = 5.6 Hz, 2H), 3.83-3.69 (m, 1H), 3.61-3.51 (m, 2H), 3.40- 3.32 (m, 1H), 2.12 (s, 3H), 1.76-1.59 (m, 1H), 1.58-1.49 (m, 1H), 1.50-1.34 (m, 4H). |
| 5-methyl-1-(2-meth- ylsulfonylethyl)pyr- azol-3-amine | 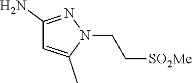 | MS (ES+) m/z = 204.3 [M + H]+, ¹H NMR (400 MHz, DMSO-D6) δ 5.19 (d, J = 0.8 Hz, 1H), 4.49 (s, 2H), 4.10 (t, J = 6.7 Hz, 2H), 3.47 (t, J = 6.7 Hz, 2H), 2.77 (s, 3H), 2.10 (d, J = 0.6 Hz, 3H). |
| 1-(2-methoxyethyl)- 5-methyl-pyrazol-3- amine | 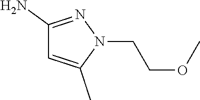 | MS (ES+) m/z = 156.2 [M + H]+. ¹H NMR (400 MHz, DMSO-D6) δ 5.17 (d, J = 0.9 Hz, 1H), 4.39 (s, 2H), 3.86 (t, J = 5.5 Hz, 2H), 3.53 (t, J = 5.5 Hz, 2H), 3.19 (s, 3H), 2.08 (d, J = 0.7 Hz, 3H). |
| 1-(2-fluoroethyl)- 5-methyl-pyrazol-3- amine | 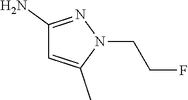 | MS (ES+) m/z = 144.0 [M + H]+. ¹H NMR (400 MHz, DMSO-D6) δ 5.22 (d, J = 0.8 Hz, 1H), 4.67 (t, J = 4.8 Hz, 1H), 4.55 (t, J = 4.8 Hz, 1H), 4.45 (s, 2H), 4.07 (t, J = 4.8 Hz, 1H), 4.00 (t, J = 4.8 Hz, 1H), 2.09 (d, J = 0.7 Hz, 3H). ¹⁹F NMR (376 MHz, DMSO-D6) δ −221.62 (tt, J = 47.5, 26.8 Hz) |
| 1-[2-(dimethyl- amino)ethyl]-5- methyl-pyrazol- 3-amine | 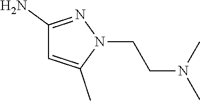 | MS (ES+) m/z = 169.1 [M + H]+, ¹H NMR (400 MHz, CHLOROFORM-D) δ 5.37 (q, J = 0.8 Hz, 1H), 3.97-3.89 (m, 2H), 3.51 (s, 2H), 2.69-2.60 (m, 2H), 2.28 (s, 6H), 2.18 (d, J = 0.7 Hz, 3H). |
| 2-(3-amino-5-meth- yl-pyrazol-1-yl) acetonitrile | 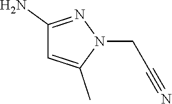 | 1H NMR (400 MHz, CHLOROFORM-D) δ 5.46 (q, J = 0.8 Hz, 1H), 4.75 (s, 2H), 3.70 (s, 2H), 2.22 (d, J = 0.9 Hz, 3H). |
| 4-methyl-1-(2-meth- ylsulfonylethyl)pyr- azol-3-amine | 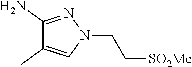 | MS (ES+) m/z = 204.1 [M + H]+. ¹H NMR (400 MHz, DMSO-D6) δ 7.18 (d, J = 1.0 Hz, 1H), 4.47 (s, 2H), 4.18 (t, J = 6.8 Hz, 2H), 3.50 (t, J = 6.8 Hz, 2H), 2.81 (d, J = 1.1 Hz, |
| | | 3H), 1.79 (t, J = 1.0 Hz, 3H). |
| 1-(2-methoxy- ethyl)-4-methyl-3- nitro-pyrazole | 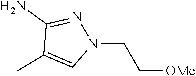 | MS (ES+) m/z = 156.0 [M + H]⁺, 1H NMR (400 MHz, CHLOROFORM-D) δ 6.99 (s, 1H), 4.00 (t, J = 5.3 Hz, 2H), 3.64 (t, J = 5.4 Hz, 2H), 3.46 (br s, 2H), 3.31 (s, 3H), 1.89 (s, 3H). |
| 1-(2-fluoroethyl)- 4-methyl-pyrazol-3- amine | 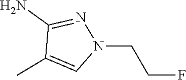 | MS (ES+) m/z = 144.0 [M + H]+. ¹H NMR (400 MHz, DMSO-d6) δ 7.13 (d, J = 1.0 Hz, 1H), 4.61 (ddd, J = 47.4, 5.1, 4.5 Hz, 2H), 4.39 (s, 2H), 4.05 (dt, J = 27.3, 4.8 Hz, 2H), 1.79 (d, J = 0.8 Hz, 3H). ¹⁹F NMR (376 MHz, |
| | | CDCl3) δ −221.05 (tt, J = 47.6, 27.4 Hz). |
| 1-[2-(dimethyl- amino)ethyl]-4-meth- yl-pyrazol-3-amine | 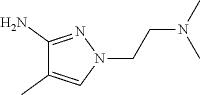 | MS (ES+) m/z = 169.1 [M + H]+, 1H NMR (400 MHz, CHLOROFORM-D) δ 7.00 (d, J = 0.9 Hz, 1H), 3.97 (t, J = 6.9 Hz, 2H), 3.45 (s, 2H), 2.68 (t, J = 6.9 Hz, 2H), 2.26 (s, 6H), 1.91 (t, J = 1.1 Hz, 3H). |
| 2-(4-amino-1H- pyrazol-1-yl)-1- (pyrrolidin-1-yl)- ethan-1-one | 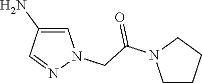 [SMILES: Nc1cnn(CC(=O)N2CCCC2)c1] | ¹H NMR (400 MHz, DMSO-D6) δ 6.97 (d, J = 0.9 Hz, 1H), 6.89 (d, J = 0.9 Hz, 1H), 4.77 (s, 2H), 3.41 (t, J = 6.8 Hz, 2H), 3.28 (t, J = 6.9 Hz, 2H), 1.96-1.82 (m, 2H), 1.76 (hd, J = 7.4, 1.0 Hz, 2H). |
| 2-(4-amino-3-meth- yl-1H-pyrazol-1-yl)- 1-(pyrrolidin-1-yl)- ethan-1-one | 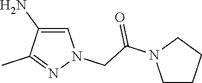 | ¹H NMR (400 MHz, DMSO-D6) δ 6.90 (s, 1H), 4.69 (s, 2H), 3.40 (t, J = 6.8 Hz, 2H), 3.27 (t, J = 6.9 Hz, 2H), 1.97 (s, 3H), 1.87 (p, J = 6.2 Hz, 2H), 1.75 (p, J = 6.6 Hz, 2H). |
| 2-(4-amino-5-meth- yl-1H-pyrazol-1-yl)- 1-(pyrrolidin-1-yl)- ethan-1-one | 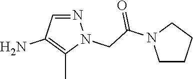 | ¹H NMR (400 MHz, DMSO-D6) δ 6.86 (s, 1H), 4.77 (s, 2H), 3.62 (s, 2H), 3.45 (t, J = 6.8 Hz, 2H), 3.28 (t, J = 6.8 Hz, 2H), 1.99 (s, 3H), 1.93-1.85 (m, 2H), 1.80-1.72 (m, 2H). |
| 4-methyl-1-(oxazol- 2-ylmethyl)pyrazol- 3-amine | 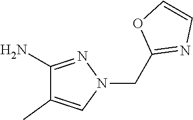 | MS (ES+) m/z = 179.1 [M + H]+; 1H NMR (400 MHz, DMSO-D6) δ 8.04 (d, J = 0.9 Hz, 1H), 7.24 (d, J = 0.9 Hz, 1H), 7.16 (d, J = 0.9 Hz, 1H), 5.11 (s, 2H), 4.45 (s, 2H), 1.80 (d, J = 0.9 Hz, 3H). |
| tert-butyl 2-(3- amino-4-methyl- pyrazol-1-yl)acetate | 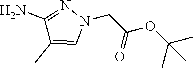 | MS (ES+) m/z = 212.2 [M + H]+. ¹H NMR (400 MHz, CHLOROFORM-D) δ 7.02 (s, 1H), 4.53 (d, J = 0.6 Hz, 2H), 3.36 (br s, 2H), 1.93 (s, 3H), 1.46 (s, 9H). |
| tert-butyl 2-(3- amino-1H-pyrazol- 1-yl)propanoate | 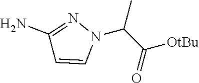 | MS (ES+) m/z = 212.1 [M + H]+, ¹H NMR (400 MHz, DMSO-D6) δ 7.36 (d, J = 2.3 Hz, 1H), 5.41 (d, J = 2.3 Hz, 1H), 4.70 (q, J = 7.3 Hz, 1H), 4.60 (s, 2H), 1.52 (d, J = 7.3 Hz, 3H), 1.36 (s, 9H). |
| tert-butyl 2-(3- amino-4-methyl- pyrazol-1-yl) propanoate | 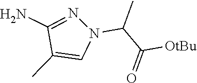 [SMILES: Cc1cn(C(C)C(=O)OC(C)(C)C)nc1N] | MS (ES+) m/z = 226.3 [M + H]+, 1H NMR (400 MHz, CHLOROFORM-D) δ 7.09 (q, J = 0.8 Hz, 1H), 4.67 (q, J = 7.4 Hz, 1H), 3.50 (s, 2H), 1.93 (d, J = 0.8 Hz, 3H), 1.64 (d, J = 7.3 Hz, 3H), 1.43 (s, 9H). |
| 2-(3-aminopyrazol- 1-yl)-1-[2-(trifluoro- methyl)pyrrolidin- 1-yl]ethanone | 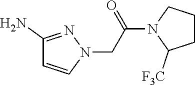 [SMILES: Nc1ccn(CC(=O)N2CCCC2C(F)(F)F)n1] | MS (ES+) m/z = 263.1 [M + H]+, ¹H NMR (400 MHz, DMSO-D6) δ 7.30 (dd, J = 39.0, 2.2 Hz, 1H), 5.95 (s, 2H), 5.49-5.39 (m, 1H), 5.03-4.54 (m, 3H), 3.63-3.14 (m, 2H), 2.16- 1.80 (m, 4H). ¹⁹F NMR (376 MHz, DMSO- D6) δ −72.47 (d, J = 8.1 Hz), −72.98 (d, J = |
| | | 8.0 Hz) |
| 2-(3-amino-4-meth- yl-pyrazol-1-yl)- 1-[2-(trifluorometh- yl)pyrrolidin-1-yl] ethanone | 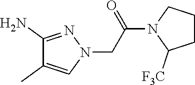 [SMILES: Cc1cn(CC(=O)N2CCCC2C(F)(F)F)nc1N] | MS (ES+) m/z = 277.1 [M + H]+, ¹H NMR (400 MHz, DMSO-D6) δ 7.29-7.05 (m, 1H), 4.93-4.51 (m, 3H), 3.63-3.49 (m, 2H), 3.29 (s, 2H), 2.08-1.84 (m, 4H), 1.80 (dd, J = 10.1, 0.8 Hz, 3H). ¹⁹F NMR (376 MHz, DMSO-D6) δ −72.47 (d, J = 8.1 Hz), −72.99 (d, J = 8.0 Hz). |

### 3-(3-Amino-1H-pyrazol-1-yl)-1-methylpyrrolidin-2-one

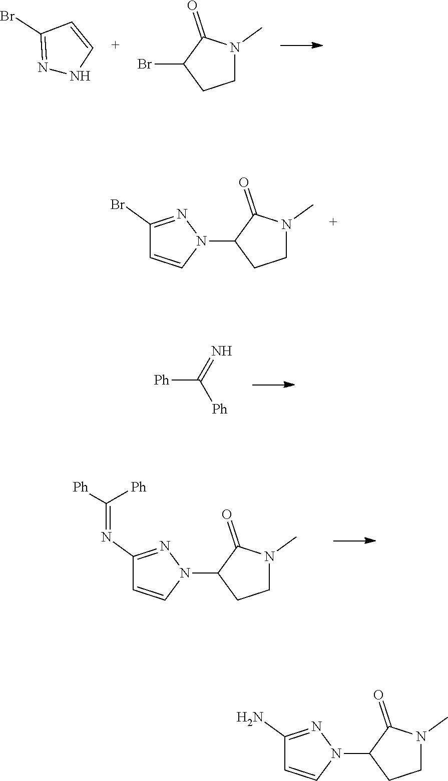

Potassium carbonate 325 mesh (3.49 eq, 1.30 g, 9.41 mmol) was added to a mixture of 3-bromo-1H-pyrazole (1.62 eq, 640 mg, 4.35 mmol) and 3-bromo-1-methylpyrrolidin-2-one (1.00 eq, 480 mg, 2.70 mmol) in MeCN (20 mL). The reaction mixture was heated at 60° C. for 18 h, cooled, filtered and evaporated. The crude material was purified by column chromatography over silica (24 g cartridge) eluting with isocratic EtOAc (5 vol) then a gradient of MeOH(0% to 20%; v/v) in EtOAc to afford 3-(3-bromopyrazol-1-yl)-1-methyl-pyrrolidin-2-one (520 mg, 2.11 mmol, 78.22% yield) as an off-white solid.MS (ES+) m/z=244.0/246.0 mono Br pattern. ¹H NMR analysis (sample reference: PT-0472-199-S2) ¹H NMR (400 MHz, CHLOROFORM-D) δ 7.50 (t, J=2.4 Hz, 1H), 6.35-6.27 (m, 1H), 4.83 (t, J=8.5 Hz, 1H), 3.60 (ddt, J=9.8, 8.7, 3.1 Hz, 1H), 3.51-3.39 (m, 1H), 2.93 (t, J=1.3 Hz, 3H), 2.76-2.53 (m, 2H).

3-(3-Bromopyrazol-1-yl)-1-methyl-pyrrolidin-2-one (520 mg, 2.13 mmol) and 4,5-bis(diphenylphosphino)-9,9-dimethylxanthene (Xantphos)(280 mg, 0.484 mmol) were dissolved in 1,4-dioxane (10 mL). Benzophenoneimine (0.60 mL, 3.58 mmol), caesium carbonate (2.00 g, 6.14 mmol) and palladium (II)acetate (0.314 eq, 150 mg, 0.668 mmol) were then added, under nitrogen. The reaction mixture was stirred at 110° C. for 18 h, then cooled, filtered through a short pad of celite and rinsed with DCM. The filtrate was concentrated. The crude material was purified by column chromatography over silica (24 g cartridge) eluting with a gradient of EtOAc (5% to 100%; v/v) in isohexane [note 3] to afford the 3-[3-(benzhydrylidene amino)pyrazol-1-yl]-1-methyl-pyrrolidin-2-one (550 mg, 1.44 mmol, 67.46% yield) as a yellow solid.MS (ES+) m/z=345.2 [M+H]+. ¹H NMR (400 MHz, CHLOROFORM-D) δ 7.84-7.78 (m, 2H), 7.41 (dd, J=5.2, 2.0 Hz, 4H), 7.39-7.32 (m, 2H), 7.27 (s, 1H), 7.25-7.19 (m, 2H), 5.02 (d, J=2.5 Hz, 1H), 4.78-4.68 (m, 1H), 3.58 (td, J=9.3, 4.0 Hz, 1H), 3.39 (ddd, J=9.7, 8.0, 6.4 Hz, 1H), 2.90 (s, 3H), 2.73 (ddt, J=13.5, 8.9, 6.8 Hz, 1H), 2.62-2.49 (m, 1H).

A slurry of palladium on activated carbon (100 mg, 0.0470 mmol) [type 39 paste] in EtOAc/EtOH [3:1](5 mL) was added under nitrogen to 3-[3-(benzhydrylideneamino)pyrazol-1-yl]-1-methyl-pyrrolidin-2-one (1.00 eq, 550 mg, 1.44 mmol) in Ethanol (5 mL) and Ethyl acetate (15 mL). The reaction mixture head space was flushed with hydrogen gas. The reaction mixture was stirred under hydrogen atmosphere for 3 days, purged with nitrogen, the catalyst was filtered off through a celite pad and washed copiously with MeCN. The filtrate was evaporated. The residue was dissolved in fresh ethanol (25 mL). Under nitrogen, a slurry of palladium hydroxide on carbon (Pd(OH) 2/C, 20% wt)(0.0991 eq, 100 mg, 0.142 mmol) in ethanol (2×5 mL) was added. The reaction mixture was stirred under hydrogen atmosphere for 7 days. The catalyst was filtered off through a celite pad and washed with EtOH. The filtrate was evaporated re-dissolved in MeOH and loaded onto an SCX-2 cartridge (5 g). The cartridge was washed with MeCN/MeOH 1:1 (25 mL), then the compound was eluted with 7 M ammonia in MeOH(25 mL). The eluent was concentrated to dryness under reduced pressure to afford 3-(3-aminopyrazol-1-yl)-1-methyl-pyrrolidin-2-one (340 mg, 1.51 mmol, 100% yield) as a brown oil. ¹H NMR (400 MHz, METHANOL-D4) δ 7.36 (d, J=2.4 Hz, 1H), 5.60 (d, J=2.4 Hz, 1H), 4.88-4.76 (m, 1H), 3.63-3.34 (m, 2H), 2.93-2.83 (m, 3H), 2.65-2.20 (m, 2H).

### Nitroaryl Amide or Sulfonamide Formation and Fe or Zn Metal Reduction of Nitro 3-(3,3,4,4-Tetrafluoropyrrolidin-1-yl)sulfonylaniline

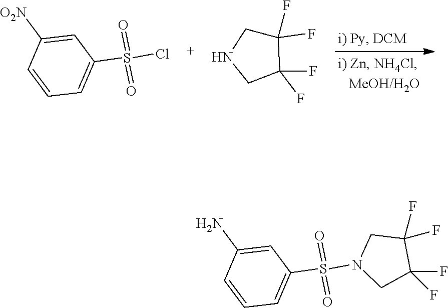 [SMILES: O=[N+]([O-])c1cccc(S(=O)(=O)Cl)c1 ; FC1(F)CNCC1(F)F ; Nc1cccc(S(=O)(=O)N2CC(F)(F)C(F)(F)C2)c1]

A mixture of 3-nitrobenzenesulfonyl chloride (136 mg, 0.613 mmol, 1.10 eq), 3,3,4,4-tetrafluoropyrrolidine hydrochloride (100 mg, 0.557 mmol, 1.00 eq), pyridine (0.14 mL, 1.67 mmol, 3.00 eq) and DCM (3.5 mL) was stirred at rt for 1 h before it was diluted with EtOAc (40 mL) and washed with 1 M HCl (2×30 mL) and sat. NaHCO₃ (2×30 mL), dried over MgSO₄ and filtered. The solvent was removed under reduced pressure and the crude was dissolved in methanol (3.6 mL) and water (0.4 mL), followed by the addition of zinc (182 mg, 2.78 mmol, 5.00 eq) and ammonium chloride (89 mg, 1.67 mmol, 3.00 eq). The mixture was stirred at rt for 5 h before it was diluted with THF (10 mL) and centrifuged. The liquid was decanted and evaporated under reduced pressure. The crude was purified by RP chromatography (MeCN/H2O 5>95%) to afford 3-(3,3,4,4-tetrafluoropyrrolidin-1-yl) sulfonylaniline (46 mg, 0.154 mmol, 28%) as a white solid. ¹H NMR (300 MHz, CDCl₃) δ 7.34 (t, J=7.9 Hz, 1H), 7.13 (ddd, J=7.7, 1.8, 1.0 Hz, 1H), 7.06 (t, J=2.1 Hz, 1H), 6.92 (ddd, J=8.1, 2.4, 0.9 Hz, 1H), 3.92 (br, 2H), 3.79-3.63 (m, 4H). MS (ES-) m/z 297.0 (M−H).

### (3-Aminophenyl)-(2-azabicyclo[2.2.1]heptan-2-yl)methanone

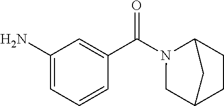 [SMILES: Nc1cccc(C(=O)N2CC3CCC2C3)c1]

Prepared similarly from i) 3-nitrobenzoyl chloride (150 mg, 0.808 mmol, 1.00 eq), 2-azabicyclo[2.2.1]heptane (102 mg, 1.05 mmol, 1.30 eq), pyridine (0.20 mL, 2.43 mmol, 3.00 eq) and DCM (3.5 mL) at rt for 2 h; ii) zinc (179 mg, 2.74 mmol, 5.00 eq), ammonium chloride (88 mg, 1.64 mmol, 3.00 eq), methanol (1.8 mL) and water (0.2 mL) at rt for 16 h to afford the title compound after triturated with isopropanol/H2O as a light brown oil (112 mg, 0.518 mmol, 94%). MS (ES+) m/z 217.5 (M+H)+.

### 3-Amino-N-(1,1,1-trifluoro-3-methylbutan-2-yl)benzamide

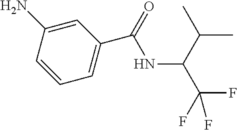 [SMILES: CC(C)C(NC(=O)c1cccc(N)c1)C(F)(F)F]

Prepared similarly from i) 3-nitrobenzoyl chloride (164 mg, 0.886 mmol, 1.00 eq), 2-methyl-1-trifluoromethyl-propylamine (125 mg, 0.886 mmol, 1.00 eq), pyridine (0.14 mL, 1.77 mmol, 2.00 eq) and DCM (4.3 mL) at rt for 1; ii) zinc (290 mg, 4.43 mmol, 5.00 eq), ammonium chloride (142 mg, 2.66 mmol, 3.00 eq), methanol (3.8 mL) and water (0.4 mL) at rt for 5 h to afford the title compound after RP chromatography (MeCN/H2O 5>95%) as white solids (49 mg, 0.0973 mmol, 25%). ¹H NMR (300 MHz, CDCl₃) δ 7.19 (m, 1H), 7.14-7.00 (m, 2H), 6.81 (d, J=7.9 Hz, 1H), 6.38 (br, 1H), 4.72 (m, 1H), 3.92 (s, 2H), 2.22 (m, 1H), 1.03 (t, J=6.9 Hz, 6H). MS (ES+) m/z 261.2 (M+H)⁺.

### (3-Aminophenyl)(3,3,4,4-tetrafluoropyrrolidin-1-yl)methanone

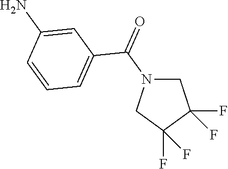 [SMILES: Nc1cccc(C(=O)N2CC(F)(F)C(F)(F)C2)c1]

Prepared similarly from i) 3,3,4,4-tetrafluoropyrrolidine hydrochloride (132 mg, 0.735 mmol, 1.00 eq), 3-nitrobenzoyl chloride (136 mg, 0.735 mmol, 1.00 eq), pyridine (0.18 mL, 2.21 mmol, 3.00 eq) and DCM (3.5 mL) at rt for 1 h; ii) zinc (240 mg, 3.68 mmol, 5.00 eq), ammonium chloride
(118 mg, 2.21 mmol, 3.00 eq), methanol (3 mL) and water (0.5 mL) at rt for 16 h to afford the title compound after RP chromatography (MeCN/H₂O 5>95%) as a light brown oil (67 mg, 0.256 mmol, 35%). H NMR (300 MHz, CDCl₃) δ 7.22-7.13 (m, 1H), 6.81-6.71 (m, 3H), 4.29-3.86 (m, 6H). MS (ES+) m/z 263.2 (M+H)⁺.

### N-(6-Aminopyridin-2-yl)-2,4,6-trifluoro-N-methylbenzamide

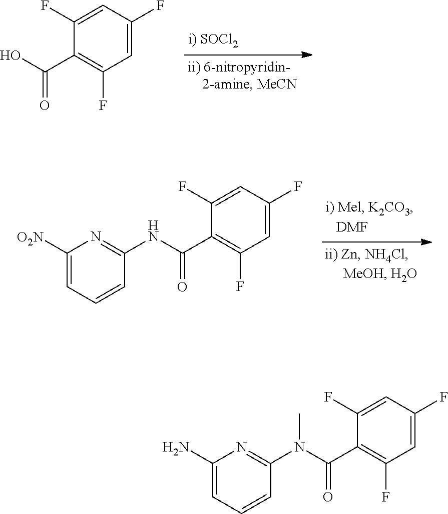 [SMILES: O=C(O)c1c(F)cc(F)cc1F ; O=C(Nc1cccc([N+](=O)[O-])n1)c1c(F)cc(F)cc1F ; CN(C(=O)c1c(F)cc(F)cc1F)c1cccc(N)n1]

A mixture of thionyl chloride (3.9 mL, 53.9 mmol, 30.0 eq) and 2,4,6-trifluorobenzoic acid
(949 mg, 5.39 mmol, 3.00 eq) was stirred at 60° C. for 1 h before it was evaporated to dryness under reduced pressure. To the formed acid chloride was added 6-nitropyridine-2-amine (250 mg, 1.80 mmol, 1.00 eq) and acetonitrile (9 mL) and the mixture was stirred at 60° C. for 3 h. After cooling to rt, the mixture was diluted with EtOAc (80 mL), washed with sat. NaHCO₃ (3×80 mL), dried over MgSO₄ and filtered. The solvent was removed under reduced pressure and the crude was purified by chromatography (EtOAc/pet ether 0>50%) to afford 2,4,6-trifluoro-N-(6-nitro-2-pyridyl)benzamide (295 mg, 0.993 mmol, 55%) as white solids. ¹H NMR (300 MHz, Methanol-d4/chloroform -d) δ 8.67 (d, J=8.2 Hz, 1H), 8.18 (t, J=8.0 Hz, 1H), 8.04 (d, J=7.8 Hz, 1H), 7.05-6.83 (m, 2H). ¹⁹F NMR (282 MHz, Methanol-d4/chloroform -d) δ-104.38--105.05 (m), -110.44-111.23 (2F, m). MS (ES-) m/z 296.2 (M−H).

A mixture of 2,4,6-trifluoro-N-(6-nitro-2-pyridyl)benzamide (137 mg, 0.461 mmol, 1.00 eq), iodomethane (0.086 mL, 1.38 mmol, 3.00 eq), potassium carbonate (194 mg, 1.38 mmol, 3.00 eq) and DMF (2.3 mL) was stirred at 80° C. for 3 h. After cooling to rt, the mixture was diluted with EtOAc (30 mL), washed with H₂O (2×30 mL), dried over MgSO₄ and filtered. The solvent was removed under reduced pressure. Zinc (241 mg, 3.69 mmol, 8.00 eq), ammonium chloride (99 mg, 1.84 mmol, 4.00 eq) methanol (12 mL) and water (3 mL) were added and the mixture was stirred vigorously for 5 h before it was filtered and washed with MeOH. The solvent was removed under reduced pressure. The crude was purified by RP chromatography (MeCN/H2O 5>95%) to afford N-(6-amino-2-pyridyl)-2,4,6-trifluoro-N-methyl-benzamide (87 mg, 0.309 mmol, 67%) as white solids. ¹H NMR (300 MHz, DMSO) δ 7.36-7.05 (m, 3H), 6.23 (d, J=8.2 Hz, 1H), 6.13 (d, J=7.5 Hz, 1H), 6.04 (s, 2H), 3.33 (s, 3H). ¹⁹F NMR (282 MHz, DMSO) δ-106.18-106.44 (m), −110.11-110.43 (2F, m). MS (ES+) m/z 282.2 (M+H)⁺.

### 4-Methyl-1-(oxazol-2-ylmethyl)-1H-pyrazol-3-amine

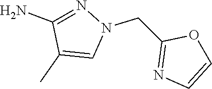

To 2-[(4-Methyl-3-nitro-pyrazol-1-yl)methyl]oxazole (186 mg, 0.893 mmol, 1 eq) and iron
(152 mg, 2.73 mmol, 3 eq) in EtOH(8 mL) and water (4 mL) at rt was added ammonium chloride
(246 mg, 4.60 mmol, 5.15 eq) and the reaction heated to 75° C. for 6 h, then filtered whilst hot (EtOH). CH2Cl2 (5 mL) and water (5 mL) were added. The phases were separated and the aqueous phase extracted with CH₂Cl₂ (2×5 mL). Combined organic phases were washed with brine (20 mL), dried over MgSO₄, filtered and concentrated in vacuo to give 4-methyl-1-(oxazol-2-ylmethyl)pyrazol-3-amine (151 mg, 0.847 mmol, 95%) which was used without further purification. MS (ES+) m/z 179 (M+H).¹H NMR (300 MHz, Chloroform-d) δ 7.60 (t, J=0.8 Hz, 1H), 7.07 (d, J=1.0 Hz, 2H), 5.14 (d, J=1.6 Hz, 2H), 3.54 (s, 2H), 1.90 (dd, J=1.9, 0.9 Hz, 3H).

### Heteroaryl Amines by Cyclisation

### 5-Cyclopropyl-1-methyl-pyrazol-3-amine

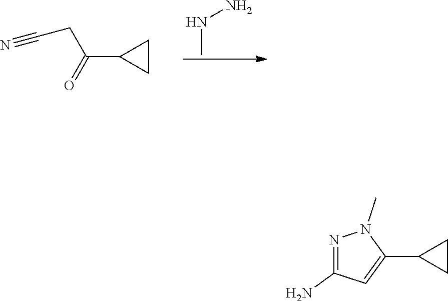

To a solution of 3-cyclopropyl-3-oxopropanenitrile (250 mg, 2.29 mmol, 1.00 equiv.) in ethanol (5 mL) was added methylhydrazine (0.24 mL, 4.58 mmol, 2.00 equiv.) and the mixture refluxed for 1 hour. The product mixture was cooled, concentrated in vacuo, quenched with water (10 mL) and extracted with EtOAc (2×10 mL). The combined organics were washed with brine (25 mL), dried over anhydrous magnesium sulfate, filtered and concentrated in vacuo to afford 5-cyclopropyl-1-methyl-pyrazol-3-amine (202 mg, 1.47 mmol, 64%). ¹H NMR (300 MHz, DMSO-d₆) δ 5.04-4.92 (m, 3H), 3.39 (s, 3H), 1.62 (tt, J=8.4, 5.0 Hz, 1H), 0.84-0.60 (m, 3H), 0.55-0.44 (m, 2H). MS (ES+) m/z 138.1 (M+H)⁺.

### 1-Methyl-4,5,6,7-tetrahydroindazol-3-amine

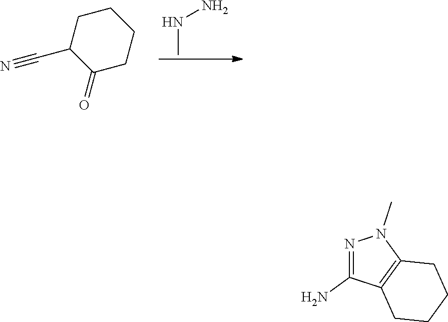

To a solution of 2-oxocyclohexane-1-carbonitrile (0.47 mL, 4.06 mmol, 1.00 eq) in EtOH(10 mL) was added methylhydrazine (0.43 mL, 8.12 mmol, 2.00 eq) and the mixture heated to reflux for 2.5 h. The mixture was cooled, concentrated in vacuo, quenched with water (10 mL), extracted with EtOAc (2×10 mL), the extracts washed with brine (25 mL), dried (MgSO₄), filtered and concentrated in vacuo to afford 1-methyl-4,5,6,7-tetrahydroindazol-3-amine (611 mg, 4.04 mmol, 100%) as a yellow oil which solidified on standing. MS (ES+) m/z 153.0 (M+H). ¹H NMR (300 MHz, DMSO-d₆) δ 4.78 (s, 2H), 3.42 (s, 3H), 2.34 (t, J=5.8 Hz, 2H), 2.21 (t, J=5.7 Hz, 2H), 1.61 (dd, J=7.4, 4.4 Hz, 4H).

### 1-Methyl-5,6-dihydro-4H-cyclopenta[c]pyrazol-3-amine

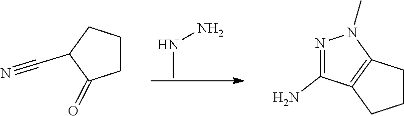

To a solution of cyclopentanone-2-carbonitrile (272 mg, 2.49 mmol, 1.00 eq) in ethanol (5 mL) was added Methylhydrazine (0.26 mL, 4.98 mmol, 2.00 eq) then heated to reflux for 20 h. The mixture was cooled, concentrated in vacuo, quenched with water (10 mL), extracted with EtOAc (2×10 mL), the extracts washed with brine (25 mL), dried (MgSO₄), filtered and concentrated in vacuo. The resulting residue was chromatographed (SiO₂) eluting with 0-5% MeOH: DCM to afford 1-methyl-5,6-dihydro-4H-cyclopenta[c]pyrazol-3-amine (120 mg, 0.875 mmol, 35%) as a beige powder.MS (ES+) m/z 138.2 (M+H). ¹H NMR (300 MHz, DMSO-d₆) δ 4.92 (s, 2H), 3.42 (s, 3H), 2.43-2.33 (m, 4H), 2.25-2.13 (m, 2H).

### N-Alkylation of Aminopyrazoles

### 1-Cyclopentyl-5-methyl-pyrazol-3-amine

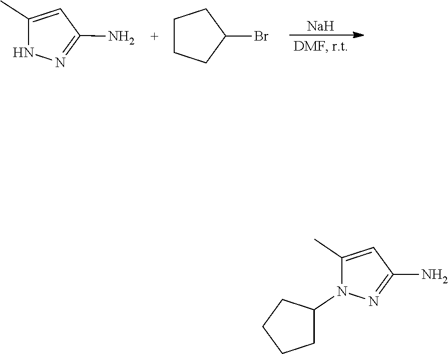

To a solution of 5-methyl-2H-pyrazol-3-yl amine (1.00 g, 10.3 mmol, 1 eq) in DMF (25 mL), sodium hydride (60% in mineral oil, 0.45 g, 11.3 mmol, 1.1 eq) was added and the reaction mixture was stirred 10 minutes at room temperature, followed the addition of bromocyclopentane (1.2 mL, 11.3 mmol, 1.1 eq). The reaction mixture was stirred at room temperature for 12 hours. After the completion, the reaction mixture was partitioned between EtOAc (50 mL) and water (100 mL). The aqueous phase was extracted 3 more times with EtOAc (50 mL). The combined organic phase was washed with water (2×50 mL), dried over MgSO₄, and concentrated under reduced pressure. The residue was purified by column chromatography (PE: EtOAc 0 to 100%) to yield 1-cyclopentyl-5-methyl-pyrazol-3-amine (430 mg, 2.60 mmol, 25%). ¹H NMR (300 MHz, DMSO-d₆) δ 5.17 (d, J=0.6 Hz, 1H), 4.46-4.29 (m, 3H), 2.10 (d, J=0.7 Hz, 3H), 1.89-1.71 (m, 6H), 1.54 (tt, J=6.2, 2.7 Hz, 2H).

### 1-Butyl-5-methyl-pyrazol-3-amine

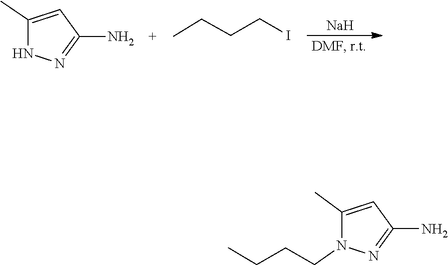

Sodium hydride (60% in mineral oil, 453 mg, 11.3 mmol, 1.10 eq) was added portion wise to a solution of 5-methyl-2H-pyrazol-3-yl amine (1.00 g, 10.3 mmol, 1 eq) in dry DMF (25 mL) under argon. The reaction mixture was stirred for 5 min, and then iodobutane (1.2 mL, 10.3 mmol, 1 eq) was added dropwise and the reaction mixture was stirred 60 h at room temperature. Upon completion, reaction mixture was cooled to room temperature and poured into water (300 mL). The aqueous layer was extracted with EtOAc (4×50 mL), and the combined organic layer was washed with brine, dried over MgSO₄, and concentrated in under reduced pressure. The crude product was purified by column chromatography (PE: EtOAc 0 to 100%) to afford 1-butyl-5-methyl-pyrazol-3-amine (320 mg, 2.09 mmol, 20%). ¹H NMR (300 MHz, Chloroform-d) δ 5.32 (s, 1H), 3.88-3.80 (m, 2H), 3.40 (s, 2H), 2.15 (s, 3H), 1.75 (p, J=7.5 Hz, 2H), 1.35 (dq, J=14.6, 7.3 Hz, 3H), 0.93 (t, J=7.3 Hz, 4H).

### 3-(5-Amino-3-methyl-pyrazol-1-yl)propanamide

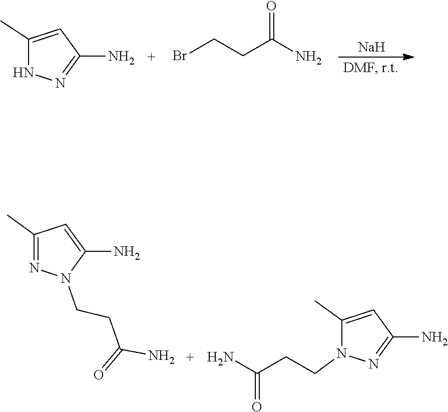

Sodium hydride (60% in mineral oil, 289 mg, 7.24 mmol, 1 eq) was added portionwise to a solution of 5-methyl-2H-pyrazol-3-yl amine (639 mg, 6.58 mmol, 1 eq) in dry DMF (25 mL) under argon. The reaction mixture was stirred for 5 min, and then 3-bromopropanamide (1.00 g, 6.58 mmol, 1 eq) was added and the reaction mixture was stirred 12 h at room temperature. Upon completion, the reaction mixture was poured into water (300 mL). The aqueous layer was extracted with EtOAc (4×50 mL), and the combined organic layer was washed with brine, dried over MgSO₄, and concentrated under reduced pressure. The crude product was purified by column chromatography (PE: EtOAc 0 to 100%) to afford 3-(3-amino-5-methyl-pyrazol-1-yl)propanamide
(300 mg, 1.78 mmol, 27%). ¹H NMR (300 MHz, Methanol-d4) δ 5.38 (s, 1H), 4.10 (t, J=6.8 Hz, 2H), 2.63 (t, J=6.8 Hz, 2H), 2.19 (s, 3H); and additionally, 3-(5-amino-3-methyl-pyrazol-1-yl)propanamide (250 mg, 1.49 mmol, 23%). MS (ES+) m/z 169.3 (M+H).

### 2-(3-Amino-4-chloro-pyrazol-1-yl)-1-pyrrolidin-1-yl-ethanone

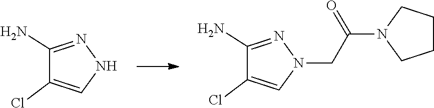 [SMILES: Nc1n[nH]cc1Cl ; Nc1nn(CC(=O)N2CCCC2)cc1Cl]

A solution of 2-chloro-1-pyrrolidin-1-yl-ethanone (1.30 g, 8.81 mmol) in MeCN (150 mL) was slowly added to a stirred suspension of 4-chloro-1H-pyrazol-3-amine (1.00 g, 8.51 mmol) and potassium carbonate 325 mesh (1.29 g, 9.36 mmol) in MeCN (100 mL) at 80° C. The reaction mixture was additionally stirred at 80° C. overnight. The reaction mixture was recharged with 2-chloro-1-pyrrolidin-1-yl-ethanone (251 mg, 1.70 mmol) and the stirring continued at 80° C. for 2 h. The reaction mixture was concentrated, preabsorbed on Celite and purified by column chromatography over silica (120 g cartridge) eluting with a gradient of MeOH(0% to 10%; v/v) in DCM (30 CV) to afford the title compound (854 mg, 3.59 mmol, 42.1% yield) as an off-white solid. MS (ES+) m/z=229.1, 230.9 [M+H]+, (mono CI pattern). ¹H NMR (400 MHz, DMSO-D6) δ 7.52 (s, 1H), 4.73 (s, 2H), 4.67 (s, 2H), 3.42 (t, J=6.8 Hz, 2H), 3.28 (t, J=6.9 Hz, 2H), 1.94-1.83 (m, 2H), 1.82-1.70 (m, 2H).

### 2-(3-Amino-4-chloro-pyrazol-1-yl)-N,N-diethyl-acetamide

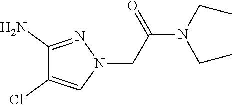

A mixture of 2-chloro-N, N-diethyl-acetamide (1.06 eq, 0.77 g, 5.15 mmol), 4-chloro-1H-pyrazol-3-amine (1 eq, 0.57 g, 4.85 mmol) and potassium carbonate 325 mesh (1.24 eq, 0.83 g, 6.01 mmol) in MeCN (10 mL) were heated at 80° C. for 4 h, then cooled, filtered and evaporated. The crude was purified by column chromatography over silica (40 g cartridge) eluting with a gradient of DCM-MeOH [9:1](0% to 80%; v/v) in DCM to afford the title compound (410 mg, 1.71 mmol, 35.18% yield) as an orange solid. MS (ES+) m/z=231.1/232.9 [M+H]+mono CI pattern. 1H NMR (400 MHz, CHLOROFORM-D) δ 7.32 (s, 1H), 4.69 (s, 2H), 3.43-3.29 (m, 4H), 1.20 (t, J=7.2 Hz, 3H), 1.12 (t, J=7.1 Hz, 3H).

### 2-[5-Amino-3-(trifluoromethyl)pyrazol-1-yl]-N, N-diethyl-acetamide

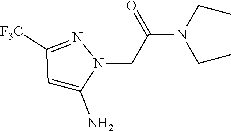 [SMILES: CCN(CC)C(=O)Cn1nc(C(F)(F)F)cc1N]

A mixture of 2-chloro-N,N-diethyl-acetamide (1.05 eq, 0.73 g, 4.88 mmol), 3-(trifluoromethyl)-1H-pyrazol-5-amine (1.00 eq, 0.70 g, 4.63 mmol) and potassium carbonate 325 mesh (1.33 eq, 0.85 g, 6.15 mmol) in MeCN (10 mL) were heated at 80° C. for 3.5h, then the reaction mixture was cooled, filtered and evaporated. The crude material was purified by column chromatography over silica (40 g cartridge) eluting with a gradient of MeOH(0% to 20%; v/v, over 20 column volumes) in DCM [Note 5] to afford 2-[5-amino-3-(trifluoromethyl)pyrazol-1-yl]-N,N-diethyl-acetamide (1.27 g, 4.33 mmol, 93.36% yield) as an orange solid. MS (ES+) m/z=265.1 [M+H]+. ¹H NMR (400 MHz, CHLOROFORM-D) § 5.79 (s, 1H), 4.86 (s, 2H), 3.46 (q, J=7.2 Hz, 2H), 3.39 (q, J=7.1 Hz, 2H), 1.24 (t, J=7.2 Hz, 3H), 1.13 (t, J=7.2 Hz, 3H). ¹⁹F NMR (376 MHz, CHLOROFORM-D) δ-62.70.

### 1-(2,2,2-Trifluoroethyl)-1H-indazol-3-amine

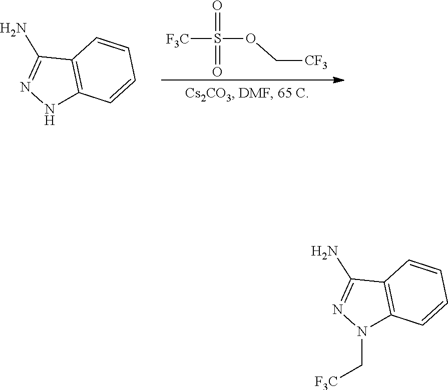 [SMILES: Nc1n[nH]c2ccccc12 ; O=S(=O)(OCC(F)(F)F)C(F)(F)F ; Nc1nn(CC(F)(F)F)c2ccccc12]

3-Amino-1H-indazole (250 mg, 1.88 mmol, 1.00 eq) was taken up in DMF (10 mL) and cesium carbonate (1224 mg, 3.76 mmol, 2.00 eq) added. The suspension was stirred for 2 mins before adding 2,2,2-trifluoroethyl trifluoromethanesulfonate (654 mg, 2.82 mmol, 1.50 eq). The reaction mixture was stirred at 65° C. for 2 days. The reaction was allowed to cool and water and ethyl acetate added and the layers separated. The aq layer was extracted into ethyl acetate (twice), and the combined organic layers were washed with water and brine (1×each). The organic layers were combined, dried (MgSO₄), filtered and concentrated to yield a brown oil that was purified on silica, eluting with Petroleum ether:EtOAc (0-100%), which yielded product as a light brown solid (130 mg). MS (ES+) m/z 216.2 (M+H)⁺. ¹H NMR (300 MHz, CDCl₃) δ 7.57 (dt, J=8.1, 1.0 Hz, 1H), 7.43 (ddd, J=8.6, 7.0, 1.1 Hz, 1H), 7.27 (d, J=8.6 Hz, 2H), 7.12 (ddd, J=8.0, 6.9, 0.9 Hz, 1H), 4.71 (q, J=8.6 Hz, 2H), 3.17 (br s, 2H).

### 1-Ethyl-5-fluoro-1H-indazol-3-amine

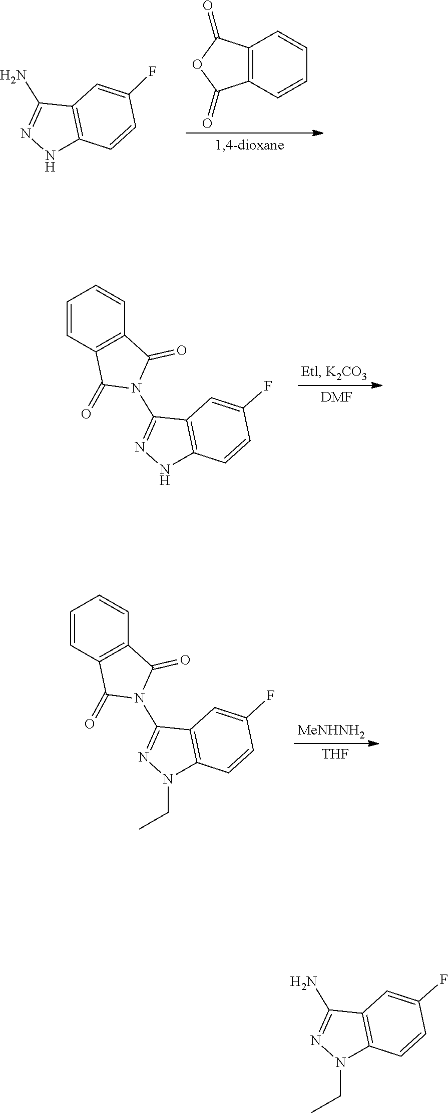

Step 1: Phthalic anhydride (1955 mg, 13.2 mmol, 4.00 eq), 5-Fluoro-1H-indazol-3-amine
(500 mg, 3.31 mmol, 1.00 eq) were taken up in 1,4-dioxane (50 mL). The solution was heated to 100° C. for 16 hours. The reaction was cooled to room temperature and concentrated under reduced pressure to leave a gum. Water (50 mL) and ethyl acetate (50 mL) were added to the residue, and the precipitated solid was collected through filtration, washed with distilled water and ethyl acetate in that order, and the cake sucked dry to afford the title compound as a light pink solid
(450 mg). MS (ES+) m/z 282.2 (M+H)+0.1H NMR (300 MHz,) δ 13.58 (s, 1H), 8.05-8.00 (m, 2H), 7.98-7.93 (m, 2H), 7.66 (dd, J=9.1, 5.0 Hz, 1H), 7.62-7.54 (m, 1H), 7.33 (td, J=9.2, 2.5 Hz, 1H).

Step 2: Potassium carbonate (383 mg, 2.73 mmol, 1.50 eq) 2-(5-fluoro-1H-indazol-3-yl) isoindoline-1,3-dione (150 mg, 0.533 mmol, 1.00 eq) and iodoethane (0.043 mL, 0.533 mmol, 1.00 eq) were stirred in DMF (7 mL) at room temp overnight. Water and EtOAc were added, and the layers separated. The aqueous layer was extracted with EtOAc (2 x), and the combined organic layers dried over MgSO₄, filtered and concentrated to yield a pink oil. This was purified on silica, eluting with 0-100% PE: EA, which yielded product as a light orange gum. MS (ES+) m/z 310.2 (M+H)⁺. ¹H NMR (300 MHz, CDCl₃) δ 8.01 (dd, J=5.6, 3.0 Hz, 2H), 7.83 (dd, J=5.5, 3.1 Hz, 2H), 7.43 (dd, J=9.8, 4.0 Hz, 1H), 7.22 (ddd, J=8.5, 6.1, 2.4 Hz, 2H), 4.48 (q, J=7.3 Hz, 2H), 1.58 (t, J=7.3 Hz, 3H); ¹⁹F NMR (282 MHz, CDCl₃) δ 121.24.

Step 3:2-(1-ethyl-5-fluoro-indazol-3-yl) isoindoline-1,3-dione (95 mg, 0.307 mmol, 1.00 eq) and Methylhydrazine (0.035 mL, 0.660 mmol, 3.00 eq) were stirred in THF (7 mL) at room temperature overnight. The solvent was evaporated, and the residue adsorbed onto isolute NH, and purified on silica gel chromatography (Pet ether/ethyl acetate (0-100%)), which yielded the product as a light orange gum. MS (ES+) m/z 180.2 (M+H)⁺.

### tert-Butyl 3-amino-4-chloro-1H-pyrazole-1-carboxylate

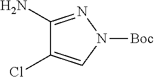 [SMILES: CC(C)(C)OC(=O)n1cc(Cl)c(N)n1]

4-Dimethylaminopyridine (20 mg, 0.164 mmol) was added to a stirring solution of 4-chloro-1H-pyrazol-3-amine (200 mg, 1.70 mmol), di-tert-butyl dicarbonate (450 mg, 2.06 mmol) and DIPEA (0.90 mL, 5.17 mmol) in DCM (15 mL). The solution was stirred at rt for 1h, sat. aq. NH₄Cl
(30 mL) was added, were separated, and the aqueous phase was extracted with DCM (2×30 mL). The combined organics were dried over Na₂SO₄ and concentrated, affording tert-butyl 3-amino-4-chloro-pyrazole-1-carboxylate (421 mg, 1.84 mmol, 107.99% yield) as a pale yellow oil. MS (ES+) m/z=117.9/119.9 (mono CI pattern) [M-Boc+H]⁺, ¹H NMR (400 MHz, CDCl₃) δ 7.85 (s, 1H), 1.61 (s, 9H).

### Bis-Aryl Amine by Suzuki Coupling of Aryl Halides with Aryl Boronic Acids (General Method P)

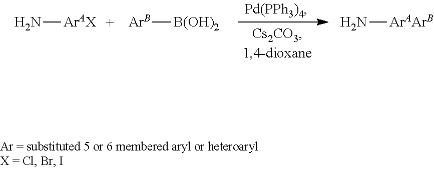

A mixture of arylboronic acid (1.0-1.5 eq), amino-aryl halide (1.0-1.5 eq), cesium carbonate
(1.1-2.0 eq), palladium (0)tetrakis(triphenylphosphine)(5-10%), and 1,4-dioxane (0.1-0.3 M) was degassed with nitrogen and stirred at reflux for 6-24 h. After cooling to rt, the mixture was diluted with H₂O and extracted with EOAc (3×). The combined organic phases were dried over MgSO₄, filtered and the solvent was removed under reduced pressure. The crude may be purified by chromatography or other methods if required.

### Representative Example: 4-(4-fluorophenyl)pyridin-2-amine

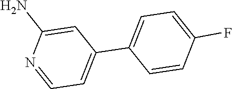 [SMILES: Nc1cc(-c2ccc(F)cc2)ccn1]

A mixture of 4-fluorobenzeneboronic acid (146 mg, 1.04 mmol, 1.20 eq), 2-amino-4-bromopyridine (150 mg, 0.867 mmol, 1.00 eq), palladium (0)tetrakis(triphenylphosphine)(100 mg, 0.0867 mmol, 0.100 eq), cesium carbonate (426 mg, 1.30 mmol, 1.50 eq) and 1,4-dioxane (4 mL) was degassed with nitrogen and stirred at 95° C. for 5 h. After cooling to rt, the mixture was diluted with H₂O (10 mL) and washed with EtOAc (3×10 mL). The combined organic phases were dried over MgSO₄, filtered and the solvent was removed under reduced pressure. The crude was purified by RP chromatography (MeCN/H₂O 5>95%) to afford 4-(4-fluorophenyl)pyridin-2-amine
(46 mg, 0.244 mmol, 28%) as a light yellow solid. ¹H NMR (300 MHz, Methanol-d4/chloroform -d) o 7.91 (d, J=5.4 Hz, 1H), 7.66-7.47 (m, 2H), 7.18-7.04 (m, 2H), 6.77 (dd, J=5.6, 1.7 Hz, 1H), 6.73 (br, 1H). MS (ES+) m/z 189.2 (M+H)⁺.

The following example compounds were prepared similarly using Method P using the appropriately substituted amine.

| Product structure and Name | Aryl halides | Aryl boronic acids | Analytical data |
|---|---|---|---|
| 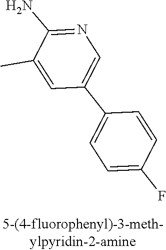 | 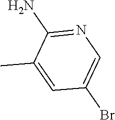 | 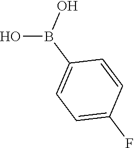 | ¹H NMR (300 MHz, Methanol- d4/chloroform-d) δ 7.97 (d, J = 2.5 Hz, 1H), 7.55-7.42 (m, 3H), 7.14-7.03 (m, 2H), 2.17 (s, 3H). MS (ES+) m/z 203.2 (M + H)⁺ |
| 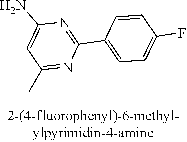 | 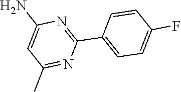 | 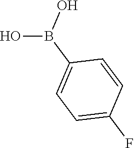 | ¹H NMR (300 MHz, Methanol- d4/chloroform-d) δ 8.30-8.05 (m, 2H), 7.23-7.03 (m, 2H), 6.25 (s, 1H), 2.34 (s, 3H). ¹⁹F NMR (282 MHz, Methanol-d4/chloro- form-d) δ −111.79 (tt, J = 8.6, 5.5 Hz). MS (ES+) m/z 204.2 (M + H)⁺ |

General Method R: Bis-Aryl Amine by Suzuki Coupling of Boc-Protected Aminoaryl Halides with Aryl Boronic Acids and Subsequent In-Situ Boc Removal

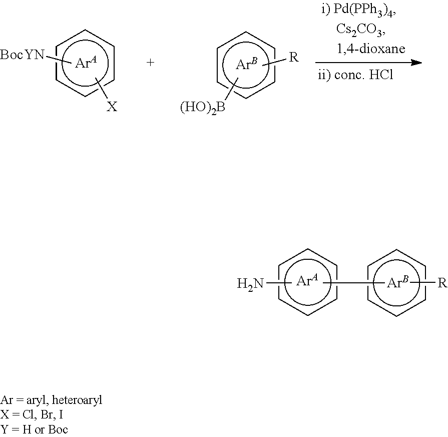

A mixture of arylboronic acid (1.0-1.5 eq), aryl halide (1.0-1.5 eq), cesium carbonate (1.1-2.0 eq), palladium (0)tetrakis(triphenylphosphine)(5%), and 1,4-dioxane (0.1-0.3 M) was degassed with nitrogen and stirred at reflux for 6-24 h. After cooling to rt, conc HCl (~1 part to 1 part 1,4-dioxane) was added and the mixture was stirred at rt for a further 1-3 h. The pH of the mixture was adjusted to 8 with 2 M NaOH and sat. NaHCO₃, followed by extraction with EOAc (3×). The combined organic phases were dried over MgSO₄, filtered and the solvent was removed under reduced pressure. The crude may be purified by chromatography or other methods if required.

### Representative Example: 6-(3,5-difluorophenyl)pyridin-2-amine

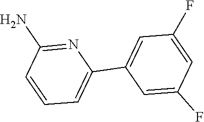 [SMILES: Nc1cccc(-c2cc(F)cc(F)c2)n1]

A mixture of tert-butyl 6-bromopyridin-2-yl carbamate (200 mg, 0.732 mmol, 1.00 eq), 3,5-difluorobenzeneboronic acid (127 mg, 0.805 mmol, 1.10 eq), palladium (0)tetrakis(triphenylphosphine)(85 mg, 0.0732 mmol, 0.100 eq), cesium carbonate (288 mg, 0.879 mmol, 1.20 eq) and 1,4-dioxane (3.5 mL) was degassed with nitrogen and stirred at 95° C. for 16 h. After cooling to rt, conc. hydrochloric acid (2.7 mL) was added and the mixture was stirred at rt for a further 3 h. The pH was adjusted to ~8 using 2 M NaOH and then sat. NaHCO₃before extraction with EtOAc (3×10 mL). The combined organic phases were dried over MgSO₄, filtered and the solvent was removed under reduced pressure. The crude was purified by RP chromatography (MeCN/H2O 5>95%) to afford 6-(3,5-difluorophenyl)pyridin-2-amine (70 mg, 0.339 mmol, 46%) as white solids. ¹H NMR (300 MHz, Methanol-d4/chloroform -d) o 7.44 (dd, J=8.3, 7.5 Hz, 1H), 7.41-7.32 (m, 2H), 6.92 (dd, J=7.4, 0.8 Hz, 1H), 6.79 (tt, J=8.8, 2.4 Hz, 1H), 6.51 (dd, J=8.3, 0.7 Hz, 1H). ¹⁹F NMR (282 MHz, Methanol-d4/chloroform -d) δ-110.60--110.79 (m). MS (ES+) m/z 207.2 (M+H)⁺.

The following example compounds were prepared similarly using Method R using the appropriately substituted amine.

| Product structure and Name | Aryl halides | Aryl boronic acids | Analytical data |
|---|---|---|---|
| 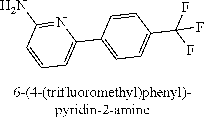 [SMILES: Nc1cccc(-c2ccc(C(F)(F)F)cc2)n1] | 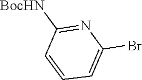 [SMILES: CC(C)(C)OC(=O)Nc1cccc(Br)n1] | 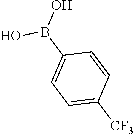 [SMILES: OB(O)c1ccc(C(F)(F)F)cc1] | ¹H NMR (300 MHz, Methanol- d4/chloroform-d) δ 7.95 (d, J = 8.1 Hz, 2H), 7.65 (d, J = 8.2 Hz, 2H), 7.49 (dd, J = 8.3, 7.4 Hz, 1H), 7.00 (dd, J = 7.4, 0.8 Hz, 1H), 6.55 (dd, J = 8.2, 0.8 Hz, 1H). ¹⁹F NMR (282 MHz, Meth- anol-d4/chloroform-d) δ −63.40- −63.45 (m). MS (ES−) m/z 237.2 |
| | | | (M − H)⁻ |
| 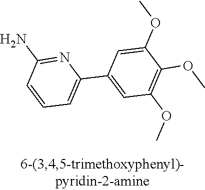 [SMILES: COc1cc(-c2cccc(N)n2)cc(OC)c1OC] | 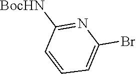 [SMILES: CC(C)(C)OC(=O)Nc1cccc(Br)n1] | 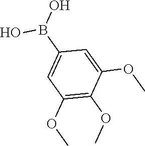 | ¹H NMR (300 MHz, Methanol- d4/chloroform-d) δ 7.44 (dd, J = 8.2, 7.5 Hz, 1H), 7.11 (s, 2H), 6.94 (dd, J = 7.5, 0.8 Hz, 1H), 6.48 (dd, J = 8.3, 0.8 Hz, 1H), 3.87 (s, 6H), 3.81 (s, 3H). MS (ES+) m/z 261.2 (M + H)⁺. |
| 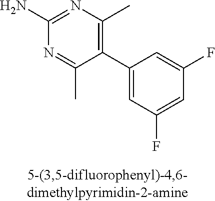 [SMILES: Cc1nc(N)nc(C)c1-c1cc(F)cc(F)c1] | 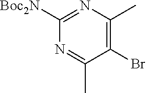 | 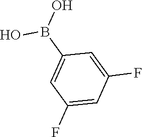 [SMILES: OB(O)c1cc(F)cc(F)c1] | ¹H NMR (300 MHz, Methanol- d4/chloroform-d) δ 6.87 (tt, J = 9.1, 2.4 Hz, 1H), 6.78-6.68 (m, 2H), 2.08 (s, 6H). ¹⁹F NMR (282 MHz, Methanol-d4/chloroform- d) δ −109.74-−109.95 (m). MS (ES+) m/z 236.6 (M + H)⁺ |

The following example compounds were prepared similarly using Method R using the appropriately substituted amine.

| Structure | Name | Analytical Data |
|---|---|---|
| 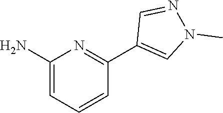 | 6-(1-methyl-1H- pyrazol-4-yl)pyridin- 2-amine | MS (ES+) m/z 175 (M + H). ¹H NMR (300 MHz, MeOD) δ 7.39 (t, J = 0.6 Hz, 1H), 7.30 (d, J = 0.8 Hz, 1H), 6.89-6.75 (m, 1H), 6.24 (dd, J = 7.4, 0.8 Hz, 1H), 5.81 (dd, J = 8.2, 0.8 Hz, 1H), 3.31 (s, 3H). |
| 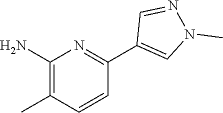 | 3-methyl-6-(1-methyl- 1H-pyrazol-4-yl)pyridin- 2-amine | MS (ES+) 189 (M + H); ¹H NMR (DMSO-d₆, 300 MHz) δ 8.01 (s, 1H), 7.80 (d, 1H, J = 0.8 Hz), 7.19 (dd, 1H, J = 7.4, 0.9 Hz), 6.73 (d, 1H, J = 7.3 Hz), 5.60 (s, 2H), 3.84 (s, 3H), 2.02 (s, 3H) ppm |
| 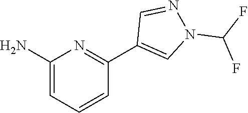 [SMILES: Nc1cccc(-c2cnn(C(F)F)c2)n1] | 6-(1-(difluoromethyl)- 1H-pyrazol-4-yl)pyridin- 2-amine | MS (ES+) m/z 211 (M + H). ¹H NMR (300 MHz, MeOD) δ 8.83 (d, J = 1.7 Hz, 1H), 8.29 (s, 1H), 7.98-7.85 (m, 1H), 7.61 (t, J = 60.9 Hz, 1H), 7.18 (dd, J = 7.6, 2.2 Hz, 1H), 6.90 (dd, J = 9.0, 1.7 Hz, 1H). |
| 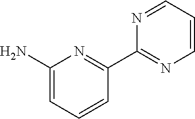 [SMILES: Nc1cccc(-c2ncccn2)n1] | 6-(pyrimidin-2-yl) pyridin-2-amine | MS (ES+) m/z 173 (M + H). ¹H NMR (300 MHz, MeOD) δ 9.32 (s, 1H), 9.20 (s, 2H), 7.99 (dd, J = 8.9, 7.4 Hz, 1H), 7.72- 7.61 (m, 1H), 7.61-7.51 (m, 1H), 7.25 (dd, J = 7.3, 0.9 Hz, 1H), 7.06 (dd, J = 8.9, 0.9 Hz, 1H). |

General Method T: Amino N-arylpyrazoles via copper-catalysed coupling of nitropyrazoles with aryl or heteroaryl halides or hypervalent aryl halides and subsequent reduction

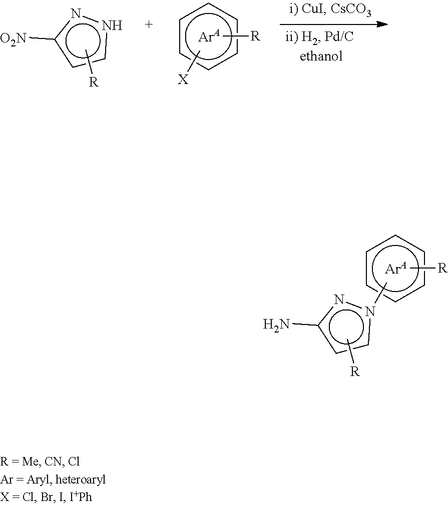

A mixture of nitropyrazole (1.0 eq), aryl or heteroaryl halide (1.0-1.2 eq), cesium carbonate
(1.1 eq) and copper (I) iodide (20 mol %), in dimethylacetamide or N-methyl pyrrolidine (0.1-0.3 M) was briefly degassed with nitrogen and stirred under microwave irradiation or conventional heating at 90-120° C. for 2-48 h. After cooling to rt, EtOAc and water were added, the phases were separated and the aqueous phase extracted with EtOAc. Combined organic phases were washed with brine dried over MgSO₄, filtered and concentrated in vacuo. Column chromatography (dichloromethane:methanol) afforded the intermediate aryl-substituted nitropyrazole which was dissolved in ethanol (0.05-0.3 M) and hydrogenated over palladium on carbon (10-30 mol %) under an atmosphere of hydrogen gas at room temperature or 40° C. for 1-16 h. The reaction was filtered through celite (ethanol) and concentrated in vacuo to give the aryl-substituted aminopyrazole which was used without further purification.

### Representative Example: 4-methyl-1-(pyridin-2-yl)-1H-pyrazol-3-amine

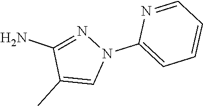

Prepared as described in Method T from 4-methyl-3-nitro-1H-pyrazole (150 mg, 1.18 mmol, 1.0 eq), 2-bromopyridine (0.11 mL, 1.18 mmol, 1.00 eq), copper (I) iodide (45 mg, 0.236 mmol, 0.2 eq) and cesium carbonate (426 mg, 1.30 mmol, 1.10 eq) in NMP (5 mL) at 120° C. for 2 h under microwave irradiation to afford 2-(4-methyl-3-nitro-pyrazol-1-yl)pyridine (195 mg, 0.96 mmol, 81%), followed by palladium on carbon (19 mg, 30 mol %) in ethanol (4 mL) at 40° C. for 16 h to afford the title compound (86 mg, 0.494 mmol, 42%) as a yellow crystalline solid. MS (ES+) m/z 175 (M+H). ¹H NMR (300 MHz, MeOD) δ 8.27 (s, 1H), 8.09 (s, 1H), 7.80 (t, J=7.7 Hz, 1H), 7.62 (d, J=7.2 Hz, 1H), 7.10 (s, 1H), 2.02 (s, 3H).

The following example compounds were prepared similarly using Method T using the appropriately substituted amine.

| Structure | Name | Analytical Data |
|---|---|---|
| 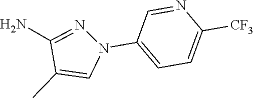 [SMILES: Cc1cn(-c2ccc(C(F)(F)F)nc2)nc1N] | 4-methyl-1-(6-(trifluoro- methyl)pyridin-3-yl)-1H- pyrazol-3-amine | MS (ES+) m/z 243 (M + H). ¹H NMR (300 MHz, MeOD) δ 8.84 (d, J = 2.6 Hz, 1H), 7.97 (dd, J = 8.6, 2.6 Hz, 1H), 7.73- 7.64 (m, 2H), 2.00 (d, J = 1.0 Hz, 3H). |
| 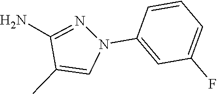 | 1-(3-fluorophenyl)-4- methyl-1H-pyrazol-3- amine | MS (ES+) m/z 242 (M + H). ¹H NMR (300 MHz, MeOD) δ 7.90-7.80 (m, 2H), 7.75 (dd, J = 8.1, 2.3 Hz, 1H), 7.53 (t, J = 8.0 Hz, 1H), 7.37 (dd, J = 7.7, 1.7 Hz, 1H), 2.01 (d, J = |
| | | 0.9 Hz, 3H). ¹⁹F NMR (282 |
| | | MHz, MeOD) δ 64.32. |
| 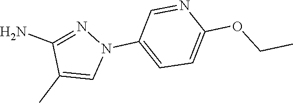 [SMILES: CCOc1ccc(-n2cc(C)c(N)n2)cn1] | 1-(6-ethoxypyridin-3-yl)- 4-methyl-1H-pyrazol-3- amine | MS (ES+) m/z 219 (M + H). ¹H NMR (300 MHz, MeOD) δ 8.30 (d, J = 2.8 Hz, 1H), 7.84 (dd, J = 8.9, 2.8 Hz, 1H), 7.65 (d, J = 1.1 Hz, 1H), 6.80 (d, J = 8.9 Hz, 1H), 4.31 (q, J = 7.1 Hz, 2H), 2.00 (d, J = 0.8 Hz, |
| | | 3H), 1.37 (t, J = 7.0 Hz, 3H). |
| 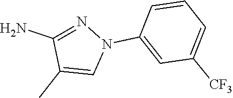 [SMILES: Cc1cn(-c2cccc(C(F)(F)F)c2)nc1N] | 4-methyl-1-(3-(trifluoro- methyl)phenyl)-1H- pyrazol-3-amine | MS (ES+) m/z 242 (M + H). ¹H NMR (300 MHz, MeOD) δ 7.90-7.80 (m, 2H), 7.75 (dd, J = 8.1, 2.3 Hz, 1H), 7.53 (t, J = 8.0 Hz, 1H), 7.37 (dd, J = 7.7, 1.7 Hz, 1H), 2.01 (d, J = |
| | | 0.9 Hz, 3H). ¹⁹F NMR (282 |
| | | MHz, MeOD) δ 64.32. |
| 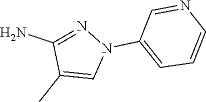 | 4-methyl-1-(pyridin-3- yl)-1H-pyrazol-3-amine | MS (ES+) m/z 175 (M + H). ¹H NMR (300 MHz, CDCl₃) δ 8.83 (dd, J = 2.8, 0.8 Hz, 1H), 8.38 (dd, J = 4.7, 1.5 Hz, 1H), 7.85 (ddd, J = 8.4, 2.7, 1.5 Hz, 1H), 7.55 (q, J = 0.9 Hz, 1H), 7.35-7.24 (m, 1H), 4.35-3.51 |
| | | (m, 2H), 2.01 (d, J = 1.0 Hz, |
| | | 3H). |
| 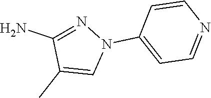 | 4-methyl-1-(pyridin-4- yl)-1H-pyrazol-3-amine | MS (ES+) m/z 175 (M + H). ¹H NMR (300 MHz, MeOD) δ 8.44-8.35 (m, 2H), 7.94 (q, J = 1.0 Hz, 1H), 7.60-7.52 (m, 2H), 2.02 (d, J = 1.0 Hz, 3H). |
| 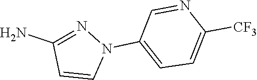 [SMILES: Nc1ccn(-c2ccc(C(F)(F)F)nc2)n1] | 1-(6-(trifluoromethyl)- pyridin-3-yl)-1H-pyr- azol-3-amine | MS (ES+) m/z 229 (M + H). ¹H NMR (300 MHz, MeOD) δ 9.02 (d, J = 2.6 Hz, 1H), 8.23- 8.10 (m, 2H), 7.82 (d, J = 8.6 Hz, 1H), 6.00 (d, J = 2.7 Hz, |
| | | 1H) ¹⁹F NMR (282 MHz, |
| | | MeOD) δ 68.66. |
| 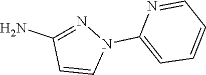 [SMILES: Nc1ccn(-c2ccccn2)n1] | 1-(pyridin-2-yl)-1H- pyrazol-3-amine | MS (ES+) m/z 161 (M + H). ¹H NMR (300 MHz, MeOD) δ 8.30 (s, 2H), 7.83 (t, J = 7.8 Hz, 1H), 7.67 (d, J = 8.4 Hz, 1H), 7.14 (s, 1H), 5.95-5.88 |
| | | (m, 1H). |
| 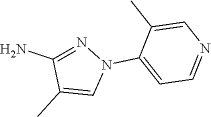 | 4-methyl-1-(3-methyl- pyridin-4-yl)-1H- pyrazol-3-amine | MS (ES+) m/z 189 (M + H). |
| 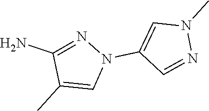 | 1′,4-dimethyl-1′H-[1,4′- bipyrazol]-3-amine | MS (ES+) m/z 178 (M + H). ¹H NMR (300 MHz, MeOD) δ 7.73 (s, 1H), 7.60 (d, J = 0.8 Hz, 1H), 7.41 (q, J = 0.8 Hz, 1H), 3.86 (s, 3H), 1.96 (d, J = 0.9 Hz, 3H). |
| 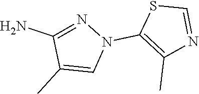 | 4-methyl-1-(4-methyl- thiazol-5-yl)-1H- pyrazol-3-amine | ¹H NMR (300 MHz, MeOD) δ 8.74 (s, 1H), 7.43 (q, J = 0.9 Hz, 1H), 2.40 (s, 3H), 2.02- 1.93 (m, 3H). |
| 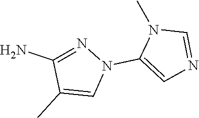 | 4-methyl-1-(1-methyl- 1H-imidazol-5-yl)-1H- pyrazol-3-amine | MS (ES+) m/z 178 (M + H). ¹H NMR (300 MHz, MeOD) δ 7.79-7.69 (m, 1H), 7.33 (q, J = 0.9 Hz, 1H), 7.02 (d, J = 1.2 Hz, 1H), 3.55 (s, 3H), 1.99 (d, J = 0.9 Hz, 3H). |
| 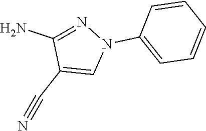 [SMILES: N#Cc1cn(-c2ccccc2)nc1N] | 3-amino-1-phenyl-1H- pyrazole-4-carbonitrile | MS (ES+) m/z 185 (M + H). |
| 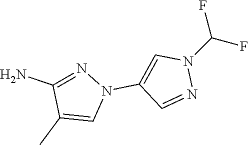 | 1′-(difluoromethyl)-4- methyl-1′H-[1,4′- bipyrazol]-3-amine | MS (ES+) m/z 214 (M + H). ¹H NMR (300 MHz, MeOD) δ 8.16 (s, 1H), 7.91 (s, 1H), 7.55 (s, 1H), 7.44 (t, J = 59.6 Hz, 1H), 1.98 (s, 3H). ¹⁹F NMR (282 MHz, MeOD) δ 96.17. |
| 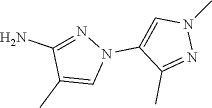 | 1′,3′,4-trimethyl-1′H- [1,4′-bipyrazol]-3- amine | MS (ES+) m/z 192 (M + H). ¹H NMR (300 MHz, MeOD) δ 7.66 (s, 1H), 7.28 (s, 1H), 3.83 (s, 3H), 2.23 (s, 3H), 2.00 (s, 3H). |
| 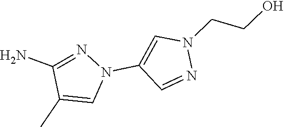 | 2-(3-amino-4-methyl- 1′H-[1,4′-bipyrazol]- 1′-yl)ethan-1-ol | MS (ES+) m/z 208 (M + H). ¹H NMR (300 MHz, MeOD) δ 7.82 (d, J = 0.8 Hz, 1H), 7.65 (d, J = 0.8 Hz, 1H), 7.44 (q, J = 0.9 Hz, 1H), 4.20 (dd, J = 5.8, 4.9 Hz, 2H), 3.88 (dd, J = 5.8, 4.9 Hz, 2H), 1.97 (d, J = 0.9 Hz, 3H). |
| 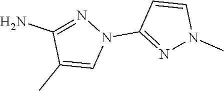 | 1′,4-dimethyl-1′H-[1,3′- bipyrazol]-3-amine | MS (ES+) m/z 178 (M + H). |

### 1′-Isopropyl-4-methyl-1′H-[1,4′-bipyrazol]-3-amine

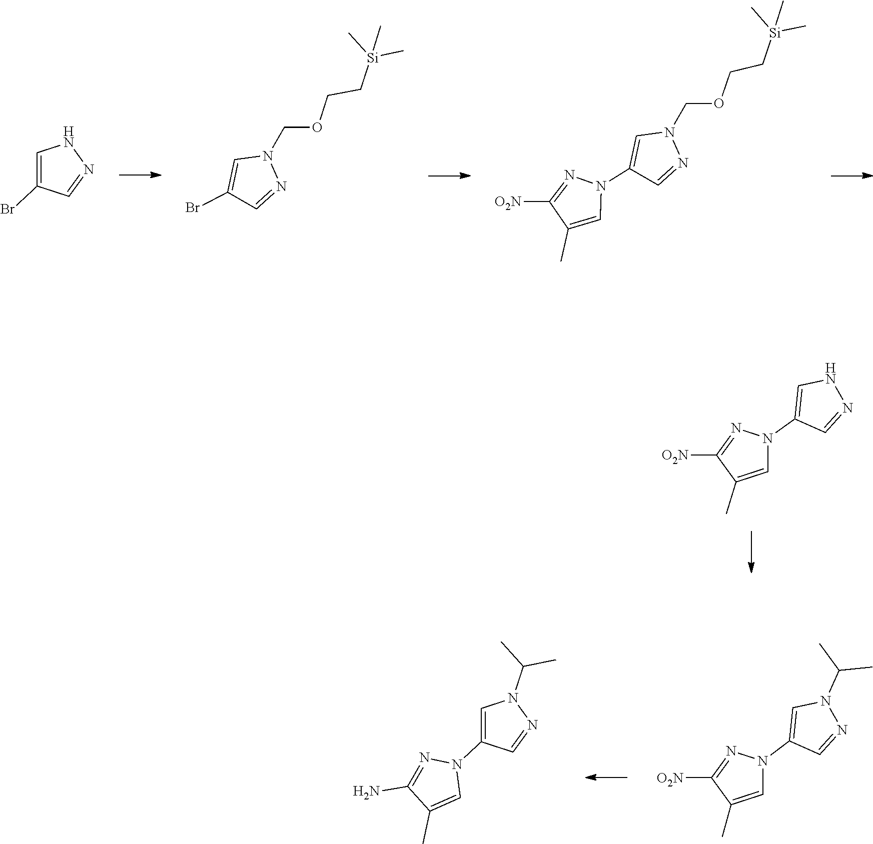 [SMILES: Brc1cn[nH]c1 ; C[Si](C)(C)CCOCn1cc(Br)cn1 ; Cc1cn(-c2cnn(COCC[Si](C)(C)C)c2)nc1[N+](=O)[O-] ; Cc1cn(-c2cn[nH]c2)nc1[N+](=O)[O-] ; Cc1cn(-c2cnn(C(C)C)c2)nc1N ; Cc1cn(-c2cnn(C(C)C)c2)nc1[N+](=O)[O-]]

Step 1: To 4-bromopyrazole (1.00 g, 6.80 mmol, 1 eq) and potassium carbonate (1.881 g, 13.6 mmol, 2 eq) in DMF (15 mL) was added dropwise 2-(chloromethoxyethyl)trimethyl silane (1.7 mL, 9.69 mmol, 1.42 eq) and the reaction stirred at rt for 6 h. EtOAc (5 mL) and water (5 mL) were added. The phases were separated and the aqueous phase extracted with EtOAc (2×5 mL). Combined organic phases were washed with water (4×10 mL), brine (20 mL), dried over MgSO₄, filtered and concentrated in vacuo. Column chromatography (dichloromethane:methanol) gave 4-bromo-1-((2-(trimethylsilyl) ethoxy)methyl)-1H-pyrazole (1497 mg, 5.40 mmol, 79%) which was used directly. MS (ES+) m/z 277, 279 (M+H).

Step 2: A mixture of 4-methyl-3-nitro-1H-pyrazole (657 mg, 5.17 mmol, 1 eq), copper (I) iodide (197 mg, 1.03 mmol, 0.2 eq) and cesium carbonate (1.864 g, 5.69 mmol, 1.10 eq) in DMA
(12 mL) was briefly degassed. 4-bromo-1-((2-(trimethylsilyl) ethoxy)methyl)-1H-pyrazole was added and the reaction heated to 120° C. for 2 h under microwave irradiation. EtOAc (5 mL) and water (5 mL) were added. The phases were separated and the aqueous phase extracted with ethyl acetate (2×5 mL). Combined organic phases were washed with brine (20 mL), dried over MgSO₄, filtered and concentrated in vacuo. Column chromatography (dichloromethane:methanol) gave 4-methyl-3-nitro-1′-((2-(trimethylsilyl) ethoxy)methyl)-1′H-1,4′-bipyrazole (267 mg, 0.826 mmol, 16%) which was used directly. ¹H NMR (300 MHz, CDCl₃) δ 8.00 (d, J=0.8 Hz, 1H), 7.81 (d, J=0.8 Hz, 1H), 7.58 (q, J=0.9 Hz, 1H), 5.44 (s, 2H), 3.64-3.52 (m, 2H), 2.40 (d, J=0.9 Hz, 3H), 0.99-0.84 (m, 2H), -0.02 (s, 9H).

Step 3: To 4-methyl-3-nitro-1′-((2-(trimethylsilyl) ethoxy)methyl)-1′H-1,4′-bipyrazole (267 mg, 0.826 mmol, 1.00 eq) in DCM (6 mL) was added trifluoroacetic acid (0.64 mL, 8.26 mmol, 10.0 eq) at rt and the reaction stirred for 5 h at rt, then concentrated in vacuo with the aid of dichloromethane (2×5 mL) to give 4-methyl-3-nitro-1-(1H-pyrazol-4-yl)pyrazole (148 mg, 0.766 mmol, 93%). MS (ES+) m/z 192 (M+H).

Step 4: To 4-methyl-3-nitro-1-(1H-pyrazol-4-yl)pyrazole (72 mg, 0.373 mmol, 1 eq) and potassium carbonate (270 mg, 1.95 mmol, 5.24 eq) in DMF (8 mL) at rt was added 2-bromopropane (0.17 mL, 1.86 mmol, 5 eq) and the reaction heated to 40° C. for 4 h. EtOAc (5 mL) and water (5 mL) were added. The phases were separated and the aqueous phase extracted with EtOAc (2×5 mL). Combined organic phases were washed with water (4×10 mL), brine (20 mL), dried over MgSO₄, filtered and concentrated in vacuo to give 1-(1-isopropylpyrazol-4-yl)-4-methyl-3-nitro-pyrazole (81 mg, 0.344 mmol, 92%) which was used without further purification. MS (ES+) m/z 236 (M+H). ¹H NMR (300 MHz, CDCl₃) δ 7.89 (d, J=0.8 Hz, 1H), 7.72 (d, J=0.8 Hz, 1H), 7.56 (q, J=0.9 Hz, 1H), 4.52 (hept, J=6.7 Hz, 1H), 2.40 (d, J=0.9 Hz, 3H), 1.53 (d, J=6.7 Hz, 6H).

Step 5:1-(1-Isopropylpyrazol-4-yl)-4-methyl-3-nitro-pyrazole (130 mg, 0.553 mmol, 1 eq) was added to palladium on carbon (13 mg, 0.121 mmol, 0.220 eq) in EtOH(5 mL) and the reaction flask evacuated and backfilled with hydrogen gas. The reaction was heated to 40° C. for 5 h, then allowed to cool and filtered through celite (ethanol) to give the title compound 1′-isopropyl-4-methyl-1′H-[1,4′-bipyrazol]-3-amine (104 mg, 0.507 mmol, 92%) which was used directly. MS (ES+) m/z 206 (M+H). ¹H NMR (300 MHz, MeOD) δ 7.83 (d, J=0.8 Hz, 1H), 7.62 (d, J=0.8 Hz, 1H), 7.44 (q, J=0.8 Hz, 1H), 4.59-4.41 (m, 1H), 1.97 (d, J=0.9 Hz, 3H), 1.50 (d, J=1.4 Hz, 3H), 1.48 (d, J=1.3 Hz, 3H).

### Halogenation of Amino-Pyrazoles

### 4-Chloro-1-(6-(trifluoromethyl)pyridin-3-yl)-1H-pyrazol-3-amine

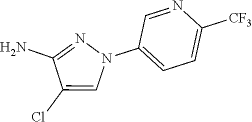 [SMILES: Nc1nn(-c2ccc(C(F)(F)F)nc2)cc1Cl]

To N-chlorosuccinimide (66 mg, 0.497 mmol, 1.05 eq) in THF (7 mL) at rt was added 1-[6-(trifluoromethyl)-3-pyridyl]pyrazol-3-amine (108 mg, 0.473 mmol, 1 eq) and the reaction stirred for 16 h rt, then concentrated in vacuo to give impure 4-chloro-1-[6-(trifluoromethyl)-3-pyridyl]pyrazol-3-amine (124 mg, 0.472 mmol, 100%) which was used without purification. MS (ES-) m/z 261 (M−H).

### 4-Chloro-1-(pyridin-2-yl)-1H-pyrazol-3-amine

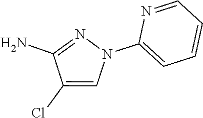 [SMILES: Nc1nn(-c2ccccn2)cc1Cl]

To 1-(2-pyridyl)pyrazol-3-amine (124 mg, 0.774 mmol, 1 eq) in THF (7 mL) at rt was added N-chlorosuccinimide (109 mg, 0.813 mmol, 1.05 eq) and the reaction stirred for 16 h rt, then concentrated in vacuo to give impure 4-chloro-1-(2-pyridyl)pyrazol-3-amine (150 mg, 0.771 mmol, 100%) which was used directly. MS (ES+) m/z 195 (M+H).

### 2.9 Diamino-Heteroaryl by SnAr of Halo-Nitroheteroaryl and Reduction of Nitro

### 6-(4,4-Difluoropiperidin-1-yl)pyridin-2-amine

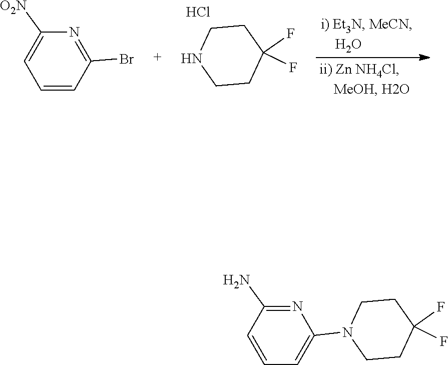 [SMILES: O=[N+]([O-])c1cccc(Br)n1 ; Cl ; FC1(F)CCNCC1 ; Nc1cccc(N2CCC(F)(F)CC2)n1]

A mixture 2-bromo-6-nitropyridine (200 mg, 0.985 mmol, 1.00 eq), 4,4-difluoropiperidine hydrochloride (186 mg, 1.18 mmol, 1.20 eq), triethylamine (0.41 mL, 2.96 mmol, 3.00 eq), acetonitrile (3 mL) water (0.5 mL) was stirred at 80° C. for 20 h. After cooling to rt, the mixture was diluted with EtOAc (30 mL), washed with 1 M HCl (30 mL) and sat. NaHCO₃ (30 mL), dried over MgSO₄ and filtered. The solvent was removed under reduced pressure. Zinc (322 mg, 4.93 mmol, 5.00 eq), ammonium chloride (158 mg, 2.96 mmol, 3.00 eq), methanol (3 mL) and water (1 mL) were added and the mixture was stirred vigorously for 2 h before it was filtered and washed with MeOH. The solvent was removed under reduced pressure and the crude was purified by RP chromatography (MeCN/H2O 5>95%) to afford 6-(4,4-difluoro-1-piperidyl)pyridin-2-amine (46 mg, 0.216 mmol, 22%) as a brown oil. ¹H NMR (300 MHz, Methanol-d4/chloroform -d) δ 7.27 (t, J=8.0 Hz, 1H), 6.04 (d, J=8.1 Hz, 1H), 5.93 (dd, J=7.7, 1.6 Hz, 1H), 3.61 (t, J=5.7 Hz, 4H), 1.97 (tt, J=13.4, 5.6 Hz, 4H). ¹⁹F NMR (282 MHz, Methanol-d4/chloroform -d) δ-96.85--97.30 (m). MS (ES+) m/z 214.2 (M+H)⁺.

### tert-Butyl(1-(6-aminopyridin-2-yl) piperidin-4-yl)carbamate

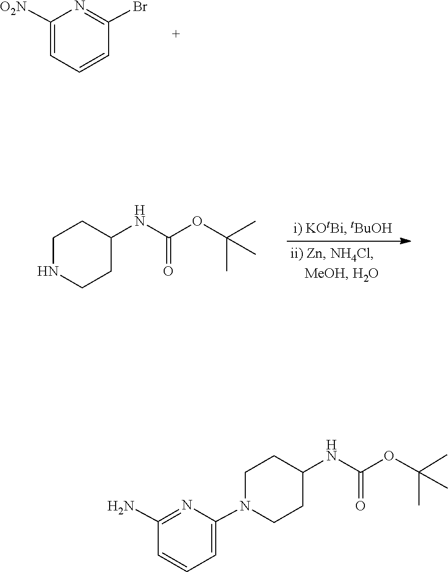 [SMILES: O=[N+]([O-])c1cccc(Br)n1 ; CC(C)(C)OC(=O)NC1CCNCC1 ; CC(C)(C)OC(=O)NC1CCN(c2cccc(N)n2)CC1]

A mixture of 2-bromo-6-nitropyridine (100 mg, 0.493 mmol, 1.00 eq), 4-N-Boc-amino-piperidine (109 mg, 0.542 mmol, 1.10 eq), potassium tert-butoxide (61 mg, 0.542 mmol, 1.10 eq) and tert-butanol (2.5 mL) was stirred at 85° C. for 16 h. After cooling to rt, H₂O (10 mL) was added, followed by extraction with EtOAc (3×10 mL). The combined organic phases were dried over MgSO₄, filtered and the solvent was removed under reduced pressure. Zinc (322 mg, 4.93 mmol, 10.0 eq), ammonium chloride (264 mg, 4.93 mmol, 10.0 eq), methanol (9 mL) and water (1 mL) were added and the mixture was stirred at rt for 3 h before it was centrifugated. The liquid was decanted and removed under reduced pressure. The crude was purified by RP chromatography (MeCN/H2O 5>95%) to afford as off white solid. ¹H NMR (300 MHz, Methanol-d4/chloroform -d) δ 7.23 (t, J=8.0 Hz, 1H), 5.98 (d, J=8.1 Hz, 1H), 5.87 (d, J=7.8 Hz, 1H), 4.13-3.94 (m, 2H), 3.54 (s, 1H), 2.94-2.75 (m, 2H), 2.04-1.81 (m, 2H), 1.48-1.29 (m, 11H). MS (ES+) m/z 293.3 (M+H)⁺.

### Diamino-Heteroaryl or Aminoalkoxy-Heteroaryl by Halogen Displacement of Halo-Aminoheteroaryl

### 2-(4,4-Difluoropiperidin-1-yl)-6-methylpyrimidin-4-amine

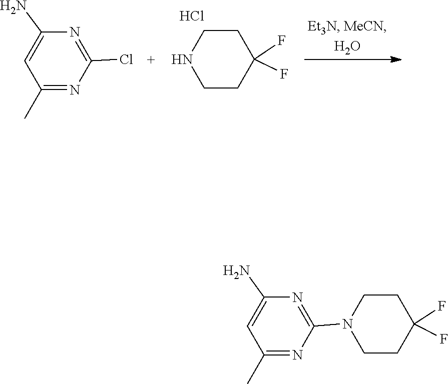

A mixture 2-chloro-6-methylpyrimidin-4-ylamine (200 mg, 1.39 mmol, 1.00 eq), 4,4-difluoropiperidine hydrochloride (263 mg, 1.67 mmol, 1.20 eq), triethylamine (0.58 mL, 4.18 mmol, 3.00 eq), acetonitrile (3 mL) and water (0.5 mL) was stirred at 80° C. for 20 h. After cooling to rt, the mixture was diluted with EtOAc (30 mL), washed with 1 M HCl (30 mL) and sat. NaHCO₃ (30 mL), dried over MgSO₄ and filtered. The solvent was removed under reduced pressure and the crude was purified by RP chromatography (MeCN/H2O 5>95%) to afford 2-(4,4-difluoro-1-piperidyl)-6-methyl-pyrimidin-4-amine (53 mg, 0.232 mmol, 17%) as white solids. ¹H NMR (300 MHz, Methanol-d4/chloroform -d) δ 5.70 (s, 1H), 3.93-3.74 (m, 4H), 2.16 (s, 3H), 2.04-1.83 (m, 4H). ¹⁹F NMR (282 MHz, Methanol-d4/chloroform -d) δ-97.03--97.36 (m). MS (ES+) m/z 229.2 (M+H)⁺.

### N²-(3,3-Dimethylbutyl)-6-(trifluoromethyl)pyrimidine-2,4-diamine

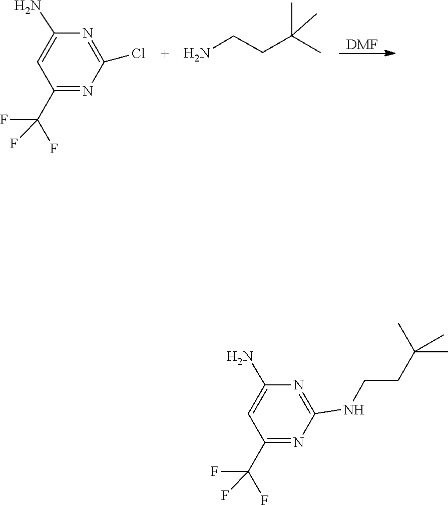

A mixture of 2-chloro-6-(trifluoromethyl)pyrimidin-4-amine (100 mg, 0.506 mmol, 1.00 eq), 3,3-dimethylbutylamine (0.27 mL, 2.02 mmol, 4.00 eq) and DMF (2.5 mL) was stirred at 80° C. for 4 h. After cooling to rt, the mixture was diluted with EtOAc (15 mL), washed with H₂O (2×10 mL), dried over MgSO₄ and filtered. The solvent was removed under reduced pressure and the crude was purified by RP chromatography (MeCN/H2O 5>95%) to afford N2-(3,3-dimethylbutyl)-6-(trifluoromethyl)pyrimidine-2,4-diamine (78 mg, 0.297 mmol, 59%) as a colourless oil. ¹H NMR
(300 MHz, CDCl₃) δ 6.04 (s, 1H), 5.27 (br, 3H), 3.46-3.23 (m, 2H), 1.55-1.35 (m, 2H), 0.99-0.82 (m, 9H). ¹⁹F NMR (282 MHz, CDCl₃) 0-71.19. MS (ES+) m/z 263.2 (M+H)⁺.

### 2-Morpholino-6-(trifluoromethyl)pyrimidin-4-amine

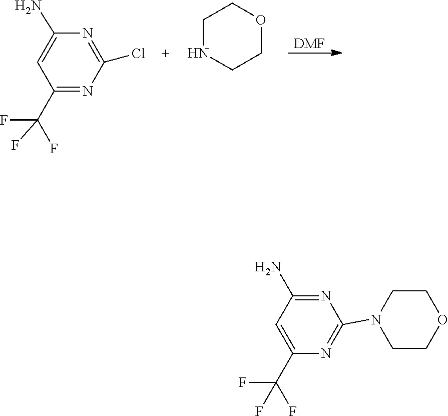

Prepared similarly from 2-chloro-6-(trifluoromethyl)pyrimidin-4-amine (120 mg, 0.607 mmol, 1.00 eq), morpholine (0.12 mL, 1.34 mmol, 2.20 eq) and DMF (3.2 mL) at 95° C. for 1 h to afford the titled compound after RP chromatography (MeCN/H2O 5>95%) as white solids (134 mg, 0.540 mmol, 89%). ¹H NMR (300 MHz, Methanol-d4/chloroform -d) δ 6.10 (s, 1H), 3.78-3.65 (m, 8H). ¹⁹F NMR (282 MHz, Methanol-d4/chloroform -d) 0-72.16. MS (ES+) m/z 249.2 (M+H)⁺.

### 2-(2,2,2-Trifluoroethoxy)-6-(trifluoromethyl)pyrimidin-4-amine

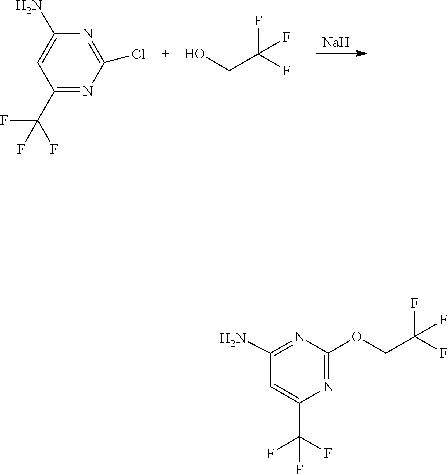

Sodium hydride (60% in mineral oil, 24 mg, 0.607 mmol, 1.20 eq) was added to a mixture of 2-chloro-6-(trifluoromethyl)pyrimidin-4-amine (100 mg, 0.506 mmol, 1.00 eq) and 2,2,2-trifluoroethanol (2.3 mL, 31.6 mmol, 62.4 eq) and the mixture was stirred at 80° C. for 20 h. After cooling to rt, the solvent was removed under reduced pressure and the crude was purified by RP chromatography (MeCN/H2O 5>95%) to afford 2-(2,2,2-trifluoroethoxy)-6-(trifluoromethyl)pyrimidin-4-amine (92 mg, 0.352 mmol, 70%) as white crystalline solids. ¹H NMR (300 MHz, Methanol-d4/chloroform -d) δ 6.55 (s, 1H), 4.78 (q, J=8.6 Hz, 2H). ¹⁹F NMR (282 MHz, Methanol-d4/chloroform -d) 0-72.03 (3F), -75.19 (3F, t, J=8.6 Hz). MS (ES+) m/z 262.2 (M+H)⁺.

### 6-Methyl-2-(2.2.2-trifluoroethoxy)pyrimidin-4-amine

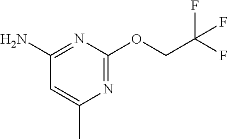

Prepared similarly from sodium hydride (60% in mineral oil, 33 mg, 0.836 mmol, 1.20 eq), 2-chloro-6-methylpyrimidin-4-ylamine (100 mg, 0.697 mmol, 1.00 eq) and 2,2,2-trifluoroethanol (2.3 mL) at 80° C. for 2 days to afford the titled compound after RP chromatography (MeCN/H2O 5>95%) as white crystalline solids (34 mg, 0.164 mmol, 24%). ¹H NMR (300 MHz, Methanol-d4/chloroform -d) δ 6.04 (d, J=0.8 Hz, 1H), 4.73 (q, J=8.7 Hz, 2H), 2.19 (d, J=0.7 Hz, 3H). 19F NMR (282 MHz, Methanol-d4/chloroform -d) 0-75.31 (t, J=8.7 Hz). MS (ES+) m/z 208.2 (M+H)⁺.

### 3-Chloro-6-(4-methylpiperazin-1-yl)pyridin-2-amine

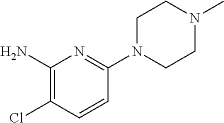

Step 1: Di-tert-butyl dicarbonate (604 mg, 2.77 mmol, 1.05 eq) was added in one portion to 6-bromo-3-chloro-pyridin-2-amine (1007 mg, 4.85 mmol, 1 eq) in dioxane (20 mL) and the reaction stirred at 40° C. for 16 h, then 90° C. for 6 h. Excess di-tert-butyl dicarbonate (604 mg, 2.77 mmol, 1.05 eq) and DMAP (5 mg, catalytic) were added and the reaction heated to 90° C. overnight then concentrated in vacuo. Column chromatography (dichloromethane:methanol) gave tert-butyl N-(6-bromo-3-chloro-2-pyridyl)-N-tert-butoxycarbonyl-carbamate (1645 mg, 4.03 mmol, 83%). MS (ES-) m/z 305, 307 (M-Boc-H). ¹H NMR (300 MHz, CDCl₃) δ 7.63 (d, J=8.3 Hz, 1H), 7.42 (dd, J=8.2, 0.6 Hz, 1H), 1.41 (d, J=1.1 Hz, 18H).

Step 2: tert-Butyl N-(6-bromo-3-chloro-2-pyridyl)-N-tert-butoxycarbonyl-carbamate (150 mg, 0.368 mmol, 1.00 eq) in N-methylpiperazine (1.0 mL, 9.01 mmol, 24.5 eq) was heated to 90° C. for 4 h. After allowing to cool, ethyl acetate (5 mL) and water (5 mL) were added. The phases were separated and the aqueous phase extracted with EtOAc (2×5 mL). Combined organic phases were washed with brine (20 mL), dried over MgSO₄, filtered and concentrated in vacuo. Column chromatography (dichloromethane:methanol) followed by treatment with trifluoroacetic acid (0.028 mL, 0.368 mmol, 1.00 eq) for 4 h at rt gave [3-chloro-6-(4-methylpiperazin-1-yl)-2-pyridyl]ammonium; 2,2,2-trifluoroacetate (36 mg, 0.106 mmol, 29%). MS (ES+) m/z 227, 229 (M+H). ¹H NMR (300 MHz, MeOD) δ 7.40 (d, J=8.5 Hz, 1H), 6.15 (d, J=8.5 Hz, 1H), 4.37 (s, 2H), 3.79-3.25 (m, 2H), 3.14 (s, 4H), 2.94 (s, 3H).

### 3-Chloro-6-morpholinopyridin-2-amine

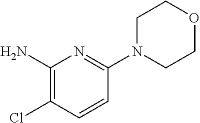 [SMILES: Nc1nc(N2CCOCC2)ccc1Cl]

Step 1: Di-tert-butyl dicarbonate (604 mg, 2.77 mmol, 1.05 eq) was added in one portion to 6-bromo-3-chloro-pyridin-2-amine (1007 mg, 4.85 mmol, 1 eq) in dioxane (20 mL) and the reaction stirred at 40° C. for 16 h, then 90° C. for 6 h. Excess di-tert-butyl dicarbonate (604 mg, 2.77 mmol, 1.05 eq) and DMAP (5 mg, catalytic) were added and the reaction heated to 90° C. overnight then concentrated in vacuo. Column chromatography (dichloromethane:methanol) gave tert-butyl N-(6-bromo-3-chloro-2-pyridyl)-N-tert-butoxycarbonyl-carbamate (1645 mg, 4.03 mmol, 83%). MS (ES-) m/z 305, 307 (M-Boc-H). ¹H NMR (300 MHz, CDCl₃) o 7.63 (d, J=8.3 Hz, 1H), 7.42 (dd, J=8.2, 0.6 Hz, 1H), 1.41 (d, J=1.1 Hz, 18H).

Step 2: A mixture of tert-butyl N-(6-bromo-3-chloro-2-pyridyl)-N-tert-butoxycarbonyl-carbamate (200 mg, 0.491 mmol, 1 eq) in morpholine (1.7 mL, 19.6 mmol, 40 eq) was heated to 90° C. for 4 h. After allowing to cool, EtOAc (5 mL) and water (5 mL) were added. The phases were separated and the aqueous phase extracted with EtOAc (2×5 mL). Combined organic phases were washed with brine (20 mL), dried over MgSO₄, filtered and concentrated in vacuo. Column chromatography (dichloromethane:methanol) followed by treatment with hydrochloric acid (2M in diethyl ether, 1 mL) for 4 h at rt gave (3-chloro-6-morpholino-2-pyridyl) ammonium chloride (41 mg, 0.164 mmol, 33%). MS (ES+) m/z 214 (M+H).¹H NMR (300 MHz, MeOD) δ 7.79 (d, J=9.1 Hz, 1H), 6.30 (d, J=9.1 Hz, 1H), 3.82 (dd, J=5.9, 3.8 Hz, 4H), 3.54 (t, J=4.9 Hz, 4H).

### 2-(4-(6-Aminopyridin-2-yl) piperazin-1-yl) ethan-1-ol. 2TFA

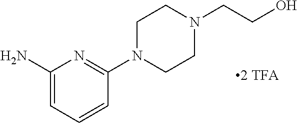 [SMILES: Nc1cccc(N2CCN(CCO)CC2)n1]

Step 1:1-(2-Hydroxyethyl) piperazine (0.12 ml, 1.01 mmol, 1.10 eq) was added to tert-butyl 6-bromopyridin-2-ylcarbamate (250 mg, 0.915 mmol, 1 eq) and the mixture heated at 115° C. for 2 h. After allowing to cool, the mixture was dissolved in DMSO (2 ml) and subject directly to reverse phase column chromatography (water:acetonitrile) to give tert-butyl N-[6-[4-(2-hydroxyethyl) piperazin-1-yl]-2-pyridyl]carbamate (37 mg, 0.115 mmol, 13%). MS (ES+) m/z 323 (M+H). ¹H NMR (300 MHz, CDCl₃) δ 7.46 (t, J=8.1 Hz, 1H), 7.20 (d, J=7.9 Hz, 1H), 6.95 (s, 1H), 6.29 (dd, J=8.3, 0.6 Hz, 1H), 3.70-3.61 (m, 2H), 3.53-3.41 (m, 4H), 2.64-2.52 (m, 7H), 1.50 (s, 9H).

Step 2: tert-Butyl N-[6-[4-(2-hydroxyethyl) piperazin-1-yl]-2-pyridyl]carbamate (37 mg, 0.115 mmol, 1.00 eq) was dissolved in CH₂Cl₂ (3 mL) and trifluoroacetic acid (6.0 mL, 77.9 mmol, 680 eq) was added dropwise. The reaction was stirred for 4 h at rt then concentrated in vacuo. Trituration with diethyl ether gave 2-[4-(6-amino-2-pyridyl) piperazin-1-yl]ethanol; 2,2,2-trifluoroacetic acid (51 mg, 0.113 mmol, 99%). MS (ES+) m/z 223 (M+H).¹H NMR (300 MHz, MeOD) δ 7.80-7.67 (m, 1H), 6.35 (dd, J=8.6, 0.7 Hz, 1H), 6.26 (dd, J=8.2, 0.8 Hz, 1H), 3.96-3.89 (m, 2H), 3.78 (s, 4H), 3.54 (d, J=11.7 Hz, 4H), 3.41-3.32 (m, 2H). ¹⁹F NMR (282 MHz, MeOD) δ 77.23.

### 2-(4-(6-Amino-5-methylpyridin-2-yl) piperazin-1-yl) ethan-1-ol

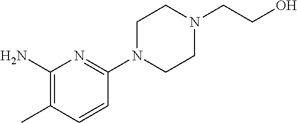 [SMILES: Cc1ccc(N2CCN(CCO)CC2)nc1N]

1-(2-Hydroxyethyl) piperazine (0.12 ml, 1.01 mmol, 1.1 eq) was added to 6-bromo-3-methylpyridin-2-amine (200 mg, 1.07 mmol, 1 eq) and the mixture heated at 115° C. for 2 h. After allowing to cool, the mixture was dissolved in DMSO (2 ml) and subject directly to reverse phase column chromatography (water:acetonitrile) to give 2-[4-(6-amino-5-methyl-2-pyridyl) piperazin-1-yl]ethanol (54 mg, 0.229 mmol, 21%). MS (ES+) m/z 238 (M+H).¹H NMR (300 MHz, MeOD) δ 7.15 (dd, J=8.0, 0.8 Hz, 1H), 6.02 (d, J=8.0 Hz, 1H), 3.74 (t, J=6.0 Hz, 2H), 3.46-3.36 (m, 4H), 2.67-2.62 (m, 4H), 2.59 (t, J=6.0 Hz, 2H), 2.03 (d, J=0.8 Hz, 3H).

### 6-(4-Cyclopropylpiperazin-1-yl)-3-methylpyridin-2-amine

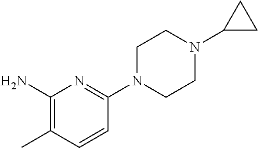 [SMILES: Cc1ccc(N2CCN(C3CC3)CC2)nc1N]

1-(2-Hydroxyethyl) piperazine (0.12 ml, 1.01 mmol, 1.1 eq) was added to 6-bromo-3-methylpyridin-2-amine (200 mg, 1.07 mmol, 1 eq) and the mixture heated at 115° C. for 2 h. After allowing to cool, the mixture was dissolved in DMSO (2 ml) and subject to reverse phase column chromatography (water:acetonitrile) to give 6-(4-cyclopropylpiperazin-1-yl)-3-methyl-pyridin-2-amine (36 mg, 0.155 mmol, 14%). MS (ES+) m/z 233 (M+H). ¹H NMR (300 MHz, MeOD) δ 7.13 (dq, J=8.0, 0.8 Hz, 1H), 6.00 (d, J=8.0 Hz, 1H), 3.36-3.32 (m, 4H), 2.75-2.70 (m, 4H), 2.01 (d, J=0.8 Hz, 3H), 1.70 (tt, J=6.6, 3.9 Hz, 1H), 0.57-0.40 (m, 4H).

### 3-((6-Aminopyridin-2-yl)(methyl)amino)propanenitrile

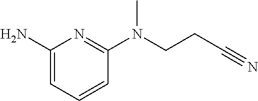

Step 1: To a mixture of 2-bromo-6-nitropyridine (300 mg, 1.48 mmol, 1 eq), 3-(methylamino)propanenitrile (0.19 mL, 2.07 mmol, 1.4 eq), 4,5-bis(diphenylphospheno)-9,9-dimethylxanthene (171 mg, 0.296 mmol, 0.2 eq) and cesium carbonate (581 mg, 1.77 mmol, 1.2 eq) in 1,4-dioxane (4 mL) and water (2 mL) was added tris(dibenzylideneacetone) dipalladium (0)
(135 mg, 0.148 mmol, 0.1 eq) and the mixture degassed with nitrogen, the heated to 90° C. for 16 h. EtOAc (5 mL) and water (5 mL) were added. The phases were separated and the aqueous phase extracted with EtOAc (2×5 mL). Combined organic phases were washed with brine (20 mL), dried over MgSO₄, filtered and concentrated in vacuo. Column chromatography (dichloromethane:methanol) gave 3-[methyl-(6-nitro-2-pyridyl)amino]propanenitrile (34 mg, 0.165 mmol, 11%). MS (ES+) m/z 207 (M+H). ¹H NMR (300 MHz, CDCl₃) δ 7.75 (dd, J=8.4, 7.6 Hz, 1H), 7.50 (dd, J=7.6, 0.5 Hz, 1H), 6.89-6.80 (m, 1H), 3.94 (t, J=6.3 Hz, 2H), 3.25 (s, 3H), 2.84 (t, J=6.2 Hz, 2H).

Step 2: Palladium on carbon (3.0 mg, 0.0282 mmol, 0.17 eq) was added to 3-[methyl-(6-nitro-2-pyridyl)amino]propanenitrile (35 mg, 0.169 mmol, 1 eq) in EtOH(5 mL) and the reaction flask evacuated and backfilled with hydrogen gas. The reaction was stirred at rt for 16 h, then allowed to cool and filtered through celite (EtOH) to give 3-[(6-amino-2-pyridyl)-methyl-amino]propanenitrile (24 mg, 0.136 mmol, 81%) which was used directly. MS (ES+) m/z 177 (M+H).

Buchwald Coupling of Boc-Protected Aryl Halides with Substituted Arylamines, Alkylamines and Amides, and Subsequent In-Situ Boc Removal (Method S).

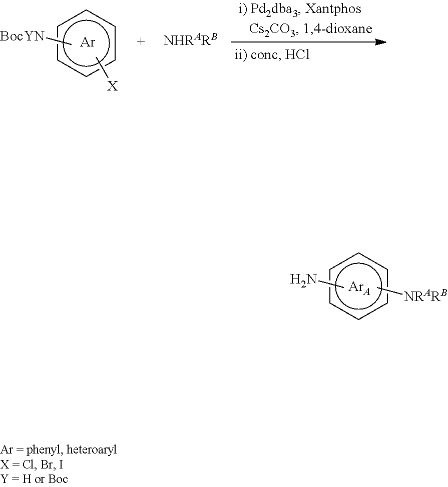

A mixture of aryl halide (1.0-1.5 eq), arylamine/alkylamine/amides (1.0-1.5 eq), cesium carbonate (1.1-2.0 eq), Pd₂dba₃ (10%), Xantphos (20%) and 1,4-dioxane (0.1-0.3 M) was degassed with nitrogen and stirred at reflux for 6-24 h. After cooling to rt, conc HCl (~1 part to 1 part 1,4-dioxane) was added and the mixture was stirred at rt for a further 1-3 h. The pH of the mixture was adjusted to 8 with 2 M NaOH and sat. NaHCO₃, followed by extraction with EOAc (3×). The combined organic phases were dried over MgSO₄, filtered and the solvent was removed under reduced pressure. The crude may be purified by chromatography or other methods if required.

### Representative Example: 5-(4,4-difluoro-1-piperidyl)pyridin-2-amine

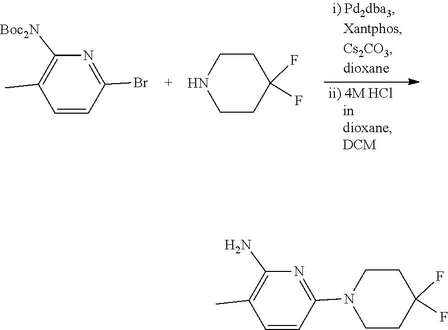

A mixture of tert-butyl N-(5-bromo-2-pyridyl)-N-tert-butoxycarbonyl-carbamate (200 mg, 0.536 mmol, 1.00 eq), 4,4-difluoropiperidine (0.072 mL, 0.643 mmol, 1.20 eq), tris(dibenzylideneacetone) dipalladium (0)(49 mg, 0.0536 mmol, 0.100 eq), 4,5-bis(diphenylphospheno)-9,9-dimethylxanthene (62 mg, 0.107 mmol, 0.200 eq), cesium carbonate
(263 mg, 0.804 mmol, 1.50 eq) and 1,4-dioxane (2.8 mL) was degassed with nitrogen and stirred at 95° C. for 5 h. After cooling to rt, conc. hydrochloric acid (1.4 mL) was added and the mixture was stirred at rt for a further 3 h before it was basified with 2 M NaOH(10 mL) and extracted with EtOAc (3×10 mL). The combined organic phases was dried over MgSO₄ and filtered. The solvent was removed under reduced pressure. The crude was purified by RP chromatography (5>95% MeCN/H2O) to afford 5-(4,4-difluoro-1-piperidyl)pyridin-2-amine (30 mg, 0.141 mmol, 26%) as white solids. ¹H NMR (300 MHz, Methanol-d4/chloroform -d) o 7.61 (dd, J=2.9, 0.8 Hz, 1H), 7.28 (dd, J=9.0, 2.9 Hz, 1H), 6.56 (dd, J=8.9, 0.8 Hz, 1H), 3.20-3.05 (m, 4H), 2.23-1.96 (m, 4H). ¹⁹F NMR (282 MHz, Methanol-d4/chloroform -d) 0-99.01 (p, J=13.8 Hz). MS (ES+) m/z 214.3 (M+H)⁺.

The following example compounds were prepared similarly using Method S using the appropriately substituted amine.

| Product structure and | | | |
|---|---|---|---|
| Name | Aryl halide | Amine/amide | Analytical data |
| 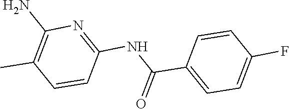 [SMILES: Cc1ccc(NC(=O)c2ccc(F)cc2)nc1N] | 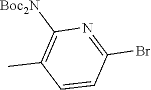 | 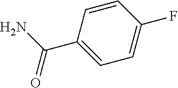 | ¹H NMR (300 MHz, Methanol- d4/chloroform-d) δ 7.97-7.87 (m, 2H), 7.41 (d, J = 7.9 Hz, 1H), 7.29 (dd, J = 7.9, 0.9 Hz, 1H), 7.19-7.06 (m, 2H), 2.07 (s, 3H). ¹⁹F NMR (282 MHz, Methanol-d4/chloroform-d) δ |
| | | | −108.24 (tt, J = 8.4, 5.3 Hz). MS |
| N-(6-amino-5-methyl-2- | | | (ES+) m/z 246.1 (M + H)⁺ |
| pyridyl)-4-fluoro- | | | |
| benzamide | | | |
| 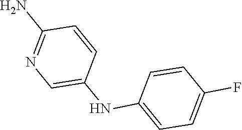 [SMILES: Nc1ccc(Nc2ccc(F)cc2)cn1] | 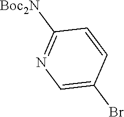 | 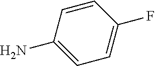 | ¹H NMR (300 MHz, Methanol- d4/chloroform-d) δ 7.73 (dd, J = 2.7, 0.8 Hz, 1H), 7.28 (dd, J = 8.8, 2.7 Hz, 1H), 6.91-6.72 (m, 4H), 6.54 (dd, J = 8.8, 0.8 Hz, 1H). ¹⁹F NMR (282 MHz, Methanol-d4/chloroform-d) δ −126.32 (tt, J = 8.6, 4.6 Hz). MS |
| | | | (ES+) m/z 204.2 (M + H)⁺ |
| N⁵-(4- | | | |
| fluorophenyl)pyridine-2,5- | | | |
| diamine | | | |
| 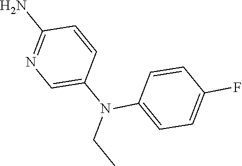 | 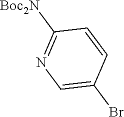 | 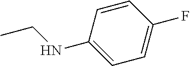 | ¹H NMR (300 MHz, Methanol- d4/chloroform-d) δ 7.69 (d, J = 2.7 Hz, 1H), 7.21 (dd, J= 8.8, 2.7 Hz, 1H), 6.90-6.79 (m, 2H), 6.71-6.62 (m, 2H), 6.58 (dd, J = 8.8, 0.8 Hz, 1H), 3.57 (q, J = 7.1 Hz, 2H), 1.14 (t, J = 7.1 Hz, 3H). ¹⁹F NMR (282 MHz, Methanol-d4/chloroform-d) δ −127.20 (m). MS (ES+) |
| | | | m/z 232.2 (M + H)⁺ |
| N5-N⁵- | | | |
| ethyl-N⁵-(4- | | | |
| fluorophenyl)pyridine-2,5- | | | |
| diamine | | | |
| 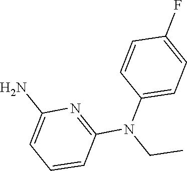 [SMILES: CCN(c1ccc(F)cc1)c1cccc(N)n1] | 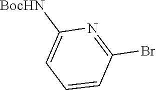 [SMILES: CC(C)(C)OC(=O)Nc1cccc(Br)n1] | 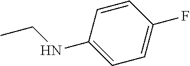 | ¹H NMR (300 MHz, Methanol- d4/chloroform-d) δ 7.23-7.13 (m, 2H), 7.13-6.98 (m, 3H), 5.87 (dd, J = 7.7, 0.7 Hz, 1H), 5.62 (dd, J = 8.1, 0.7 Hz, 1H), 3.87 (q, J = 7.0 Hz, 2H), 1.15 (t, J = 7.1 Hz, 3H). ¹⁹F NMR (282 MHz, Methanol- d4/chloroform-d) δ −117.94 (tt, J = 8.3, 5.0 Hz). MS (ES+) m/z 232.3 (M + H)⁺ |
| N²-ethyl-N²-(4- | | | |
| fluorophenyl)pyridine-2,6- | | | |
| diamine | | | |
| 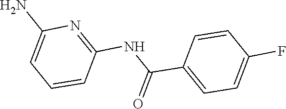 [SMILES: Nc1cccc(NC(=O)c2ccc(F)cc2)n1] | 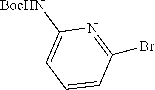 [SMILES: CC(C)(C)OC(=O)Nc1cccc(Br)n1] | 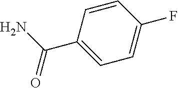 | ¹H NMR (300 MHz, Methanol- d4/chloroform-d) δ 8.00-7.88 (m, 2H), 7.53-7.37 (m, 2H), 7.24-7.10 (m, 2H), 6.33 (dd, J = 7.3, 1.5 Hz, 1H). ¹⁹F NMR (282 MHz, Methanol- d4/chloroform-d) δ −107.82- |
| | | | −108.13 (br). MS (ES−) m/z 230.2 |
| N-(6-aminopyridin-2-yl)-4- | | | (M − H)⁻ |
| fluorobenzamide | | | |
| 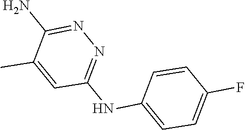 [SMILES: Cc1cc(Nc2ccc(F)cc2)nnc1N] | 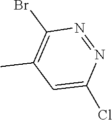 | 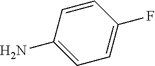 | ¹⁹F NMR (282 MHz, Methanol- d4/chloroform-d) δ −121.95- −124.75 (m). MS (ES+) m/z 219.2 (M + H)⁺ |
| N³-(4-fluorophenyl)-5- | | | |
| methylpyridazine-3,6- | | | |
| diamine | | | |
| 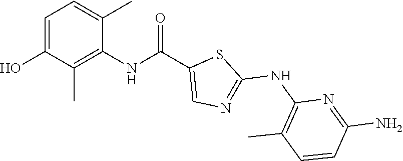 [SMILES: Cc1ccc(N)nc1Nc1ncc(C(=O)Nc2c(C)ccc(O)c2C)s1] | 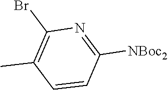 | 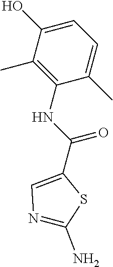 | ¹H NMR (300 MHz, Methanol- d4/chloroform-d) δ 8.08 (s, 1H), 7.23 (dd, J = 7.9, 0.8 Hz, 1H), 6.90 (d, J = 8.2 Hz, 1H), 6.68 (d, J = 8.2 Hz, 1H), 6.14 (d, J = 7.9 Hz, 1H), 2.19 (s, 3H), 2.16 (s, 3H), 2.12 (s, 3H). MS (ES+) 370.2 m/z (M + H)⁺ |
| 2-((6-amino-3- | | | |
| methylpyridin-2-yl)amino)- | | | |
| N-(3-hydroxy-2,6- | | | |
| dimethylphenyl)thiazole-5- | | | |
| carboxamide | | | |
| BAA-337 | | | |

### Acylation of Boc-Monoprotected Diamine and Boc Deprotection

### N-(6-Aminopyridin-3-yl)-2.4.6-trifluorobenzamide

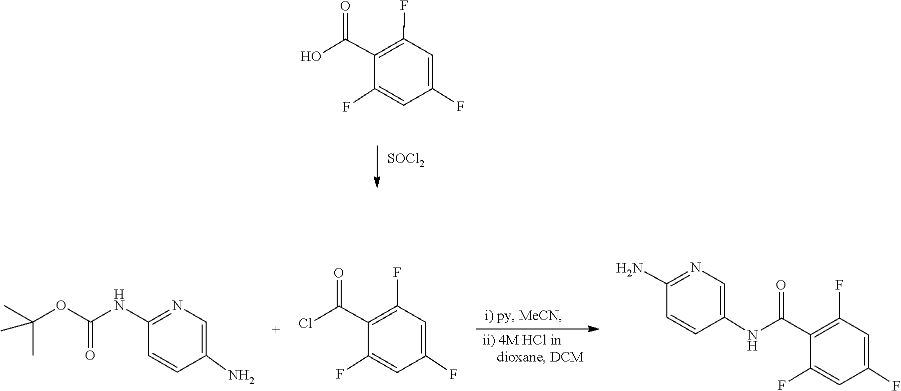

A mixture of 2,4,6-trifluorobenzoic acid (842 mg, 4.78 mmol, 5.00 eq) and thionyl chloride
(3.0 mL, 41.4 mmol, 43.3 eq) was stirred at 50° C. for 1 h and the solvent was removed under reduced pressure. To the crude acid chloride was added tert-butyl(5-aminopyridin-2-yl)carbamate
(200 mg, 0.956 mmol, 1.00 eq), acetonitrile (2.5 mL) and pyridine (0.77 mL, 9.56 mmol, 10.0 eq) and the mixture was stirred at 60° C. for 1 h. After cooling to rt, the mixture was diluted with EtOAc
(50 mL) and washed with sat. NaHCO₃ (3×40 mL), dried over MgSO₄, filtered and the solvent was removed under reduced pressure. The crude was dissolved in DCM (2 mL), followed by the addition of hydrochloric acid (4.0 M in dioxane, 2.0 mL) and the mixture was stirred at rt for a further 5 h before it was diluted with EtOAc (20 mL). The precipitated solids were collected by filtration before they were resuspended in EtOAc (50 mL) and shaken with 0.5 M NaOH(50 mL). The organic phase was washed further with H₂O (50 mL), dried over MgSO₄, filtered and the solvent was removed under reduced pressure to afford N-(6-amino-3-pyridyl)-2,4,6-trifluoro-benzamide (151 mg, 0.565 mmol, 59%) as white solids. ¹H NMR (300 MHz, Methanol-d4/chloroform -d) δ 8.13 (d, J=2.7 Hz, 1H), 7.80 (dd, J=8.9, 2.6 Hz, 1H), 6.86-6.75 (m, 2H), 6.57 (dd, J=8.9, 0.8 Hz, 1H). MS (ES+) m/z 268.5 (M+H)⁺.

### N-(6-Aminopyridin-3-yl)-3-methylbutanamide

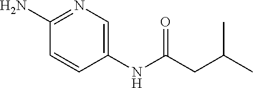

Prepared similarly (excluding acyl chloride formation) from i) tert-butyl(5-aminopyridin-2-yl)carbamate (200 mg, 0.956 mmol, 1.00 eq), isovaleryl chloride (0.15 mL, 1.24 mmol, 1.30 eq), pyridine (0.15 mL, 1.91 mmol, 2.00 eq) and acetonitrile (2.5 mL) at 60° C. for 1 h; ii) hydrogen chloride (4.0 M in dioxane, 2.0 mL), DCM (2 mL) at rt for 5 h to afford the titled compound as white solids (97 mg, 0.502 mmol, 53%). ¹H NMR (300 MHz, Methanol-d4/chloroform -d) δ 7.97 (dd, J=2.6, 0.8 Hz, 1H), 7.70 (dd, J=8.9, 2.6 Hz, 1H), 6.51 (dd, J=8.9, 0.8 Hz, 1H), 2.20-2.07 (m, 3H), 0.97 (s, 3H), 0.95 (s, 3H). MS (ES+) m/z 194.6 (M+H)⁺.

### N-(6-Aminopyridin-3-yl)-2,4-dimethylbenzamide

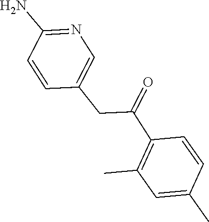 [SMILES: Cc1ccc(C(=O)Cc2ccc(N)nc2)c(C)c1]

Prepared similarly (excluding acyl chloride formation) from i) tert-butyl(5-aminopyridin-2-yl)carbamate (200 mg, 0.956 mmol, 1.00 eq), 2,4-dimethyl-benzoyl chloride (193 mg, 1.15 mmol, 1.20 eq), pyridine (0.15 mL, 1.91 mmol, 2.00 eq) and acetonitrile (2.5 mL) at 60° C. for 1 h; ii) hydrochloric acid (4.0 M in dioxane, 2.0 mL) and DCM (2 mL) at rt for 5 h to afford the titled compound as a pink solid (140 mg, 0.580 mmol, 61%). ¹H NMR (300 MHz, Methanol-d4/chloroform -d) δ 8.15 (dd, J=2.6, 0.8 Hz, 1H), 7.77 (dd, J=8.9, 2.6 Hz, 1H), 7.35 (d, J=7.6 Hz, 1H), 7.11-7.01 (m, 2H), 6.60 (dd, J=8.9, 0.8 Hz, 1H), 2.41 (s, 3H), 2.33 (s, 3H). MS (ES+) m/z 242.6 (M+H)⁺.

### Heteroaryl Halide Building Blocks

General Method O: Amide Formation from Aminopyridines and Acyl Chloride/Carboxylic Acids

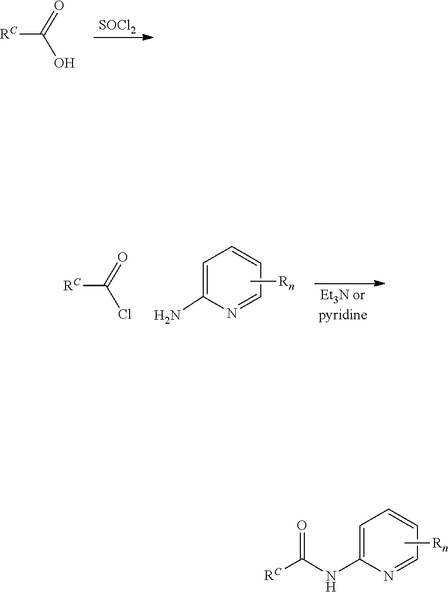

Acyl chloride formation: a mixture of carboxylic acid (1.0 eq) and thionyl chloride (0.1-0.2 M) was stirred at 50-60° C. for 0.5-2 h before the solvent was removed under reduced pressure. The crude acid chloride was taken forward immediately.

Amide formation: a mixture of amine (1.0 eq), acyl chloride (1.0-2.0 eq), triethylamine or pyridine (2.0-3.0 eq) and solvent (MeCN or DCM or 1,4-dioxane, 0.1-0.3 M) was stirred at rt for 1-16 h before it was diluted with H₂O and extracted with EtOAc (3×10 mL) [Alternative workup: for non acid-sensitive compounds, an aq. HCl wash can be incorporated to remove residual pyridine]. The combined organic phases was dried over MgSO₄ and filtered. The solvent was removed under reduced pressure. The crude could be purified by chromatography or other methods if necessary.

### N-(6-Bromo-5-methoxy-2-pyridyl)-2,2-dimethyl-propanamide

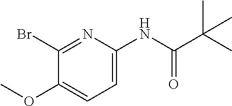 [SMILES: COc1ccc(NC(=O)C(C)(C)C)nc1Br]

A mixture of 6-bromo-5-methoxypyridin-2-amine (200 mg, 0.985 mmol, 1.00 eq), trimethylacetyl chloride (0.24 mL, 1.97 mmol, 2.00 eq), pyridine (0.16 mL, 1.97 mmol, 2.00 eq) and DCM (5 mL) was stirred at rt for 3 h before it was diluted with DCM (10 mL), washed with 0.5 M HCl (2×15 mL), sat. NaHCO₃ (15 mL), dried over MgSO₄ and filtered. The solvent was removed under reduced pressure to afford N-(6-bromo-5-methoxy-2-pyridyl)-2,2-dimethyl-propanamide (280 mg, 0.975 mmol, 99%) as light pink solids. ¹H NMR (300 MHz, CDCl₃) δ 8.14 (d, J=8.8 Hz, 1H), 8.08 (br, 1H), 7.21 (s, 1H), 3.85 (s, 3H), 1.25 (s, 9H). MS (ES-) m/z 285.1/287.0 (M−H)⁻.

The following example compounds were prepared similarly using Method O using the appropriately substituted amine.

| | | Acyl | |
|---|---|---|---|
| | | chloride/ | |
| | | carboxylic | |
| Structure and name | Amine | acid | Analytical data |
| 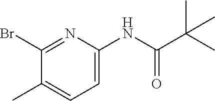 | 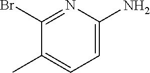 | 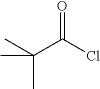 | ¹H NMR (300 MHz, CDCl₃) δ 8.05 (d, J = 8.2 Hz, 1H), 7.96 (br, 1H), 7.45 (d, J = 8.2 Hz, 1H), 2.27 (s, 3H), 1.25 (s, 9H). MS (ES+) m/z 271.2 (M + H)⁺. |
| N-(6-bromo-5-methyl-2- | | | |
| pyridyl)-2,2-dimethyl- | | | |
| propanamide | | | |
| 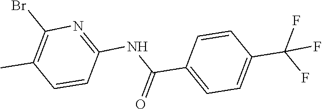 [SMILES: Cc1ccc(NC(=O)c2ccc(C(F)(F)F)cc2)nc1Br] | 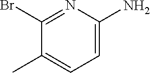 | 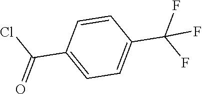 [SMILES: O=C(Cl)c1ccc(C(F)(F)F)cc1] | ¹H NMR (300 MHz, Methanol- d4/chloroform-d) δ 8.15 (d, J = 8.2 Hz, 1H), 8.10-8.03 (m, 2H), 7.79-7.73 (m, 2H), 7.63 (dd, J = 8.2, 0.8 Hz, 1H), 2.35 (s, 3H). ¹⁹F NMR (282 MHz, Methanol-d4/chloroform-d) δ |
| | | | −63.77. MS (ES+) |
| N-(6-bromo-5-methylpyridin-2- | | | m/z 359.1/361.1 (M + H)⁺ |
| yl)-4- | | | |
| (trifluoromethyl)benzamide | | | |
| 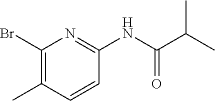 | 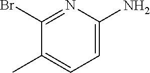 | 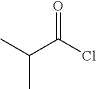 | ¹H NMR (300 MHz, CDCl₃) δ 8.11 (d, J = 8.2 Hz, 1H), 7.93 (br, 1H), 7.54 (d, J = 8.2 Hz, 1H), 2.52 (h, J = 6.9 Hz, 1H), 2.36 (s, 3H), 1.28 (s, 3H), 1.25 (s, 3H). MS (ES+) m/z 257.0/259.0 (M + H)⁺ |
| N-(6-bromo-5-methylpyridin-2- | | | |
| yl)isobutyramide | | | |
| 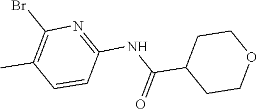 [SMILES: Cc1ccc(NC(=O)C2CCOCC2)nc1Br] | 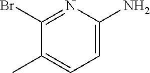 | 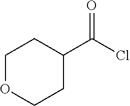 | ¹H NMR (300 MHz, CDCl₃) δ 8.10 (d, J = 8.2 Hz, 1H), 7.99 (s, 1H), 7.55 (d, J = 8.2 Hz, 1H), 4.08 (ddd, J = 11.4, 3.7, 2.5 Hz, 2H), 3.54-3.40 (m, 2H), 2.59- 2.43 (m, 1H), 2.36 (s, 3H), 2.02- 1.78 (m, 4H). MS (ES+) m/z |
| | | | 299.0/301.0 (M + H)⁺ |
| N-(6-bromo-5-methylpyridin-2- | | | |
| yl)tetrahydro-2H-pyran-4- | | | |
| carboxamide | | | |
| 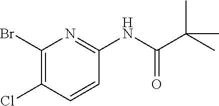 | 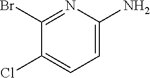 | 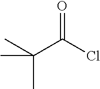 | ¹H NMR (300 MHz, CDCl₃) δ 8.21 (d, J = 8.7 Hz, 1H), 8.06 (br, 1H), 7.71 (dd, J = 8.7, 0.5 Hz, 1H), 1.31 (s, 9H). MS (ES+) m/z 291.2/293.2 (M + H)⁺ |
| N-(6-bromo-5-chloropyridin-2- | | | |
| yl)pivalamide | | | |
| 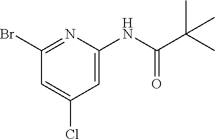 | 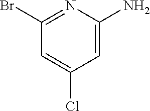 | 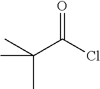 | ¹H NMR (300 MHz, CDCl₃) δ 8.32 (d, J = 1.6 Hz, 1H), 8.07 (br, 1H), 7.24 (d, J = 1.5 Hz, 1H), 1.31 (s, 9H). MS (ES+) m/z 291.2/293.2 (M + H)⁺ |
| N-(6-bromo-4-chloropyridin-2- | | | |
| yl)pivalamide | | | |
| 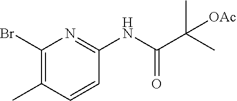 [SMILES: CC(=O)OC(C)(C)C(=O)Nc1ccc(C)c(Br)n1] | 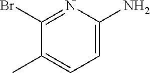 | 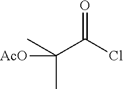 | ¹H NMR (300 MHz, CDCl₃) 8.32 (br, 1H), 8.11 (d, J = 8.2 Hz, 1H), 7.54 (d, J = 8.2 Hz, 1H), 2.35 (s, 3H), 2.15 (s, 3H), 1.69 (s, 6H). MS (ES+) m/z 315.2/317.2 (M + H)⁺ |
| 1-((6-bromo-5-methylpyridin-2- | | | |
| yl)amino)-2-methyl-1- | | | |
| oxopropan-2-yl acetate | | | |

### N-(6-Bromo-5-methylpyridin-2-yl)-2-methylpropane-2-sulfonamide

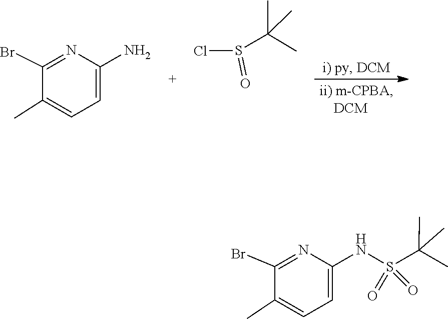

A mixture of 6-bromo-5-methylpyridin-2-amine (300 mg, 1.60 mmol, 1.00 eq), pyridine (0.26 mL, 3.21 mmol, 2.00 eq), t-butylsulfinyl chloride (0.40 mL, 3.21 mmol, 2.00 eq) and DCM (9 mL) was stirred at rt for 3 h before it was diluted DCM (6 mL) and washed with 0.5 M HCl (15 mL) and sat. NaHCO₃ (15 mL), dried over MgSO₄ and filtered. The solvent was removed under reduced pressure. DCM (15 mL) was added to the crude, followed by 3-chloroperoxybenzoic acid (70%, 593 mg, 2.41 mmol, 1.50 eq) and the mixture was stirred at rt for a further 1 h before it was washed with 0.5 M HCl (15 mL), sat. NaHCO₃ (15 mL) and sat. Na₂S203 (15 mL), dried over MgSO₄ and filtered. The solvent was removed under reduced pressure and the crude was purified by RP chromatography (MeCN/H2O 5>95%) to afford N-(6-bromo-5-methyl-2-pyridyl)-2-methyl-propane-2-sulfonamide (190 mg, 0.618 mmol, 39%) as beige solids. ¹H NMR (300 MHz, CDCl₃) δ 7.50 (br, 1H), 7.43 (d, J=8.2 Hz, 1H), 7.37 (d, J=8.2 Hz, 1H), 2.27 (s, 3H), 1.40 (s, 9H). MS (ES+) m/z 307.0/309.0 (M+H)⁺.

### 1-(6-Bromo-5-methylpyridin-2-yl)-3-(tert-butyl)urea

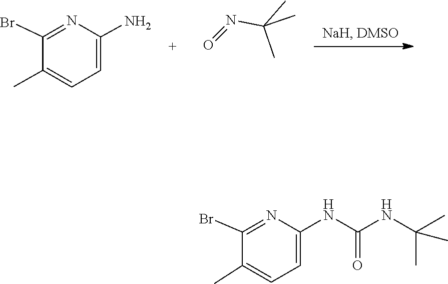

A mixture of tert-butyl isocyanate (103 mg, 1.04 mmol, 1.30 eq), 6-bromo-5-methylpyridin-2-amine (150 mg, 0.802 mmol, 1.00 eq), sodium hydride (60% in mineral oil, 42 mg, 1.04 mmol, 1.30 eq) and DMSO (4 mL) was stirred at 70° C. for 1 h. After cooling to rt, the mixture was neutralised with 2 M HCl (15 mL) and purified by RP chromatography (MeCN/H2O 5>95%) to afford 1-(6-bromo-5-methyl-2-pyridyl)-3-tert-butyl-urea (150 mg, 0.524 mmol, 65%) as white solids. ¹H NMR (300 MHz, CDCl₃) δ 8.99-8.79 (m, 2H), 7.38 (d, J=8.2 Hz, 1H), 6.74 (d, J=8.1 Hz, 1H), 2.29 (s, 3H), 1.46 (s, 9H). MS (ES+) m/z 286.0/288.0 (M+H)⁺.

### tert-Butyl(6-bromo-5-methylpyridin-2-yl)(tert-butoxycarbonyl)carbamate and tert-butyl(6-bromo-5-methylpyridin-2-yl)carbamate

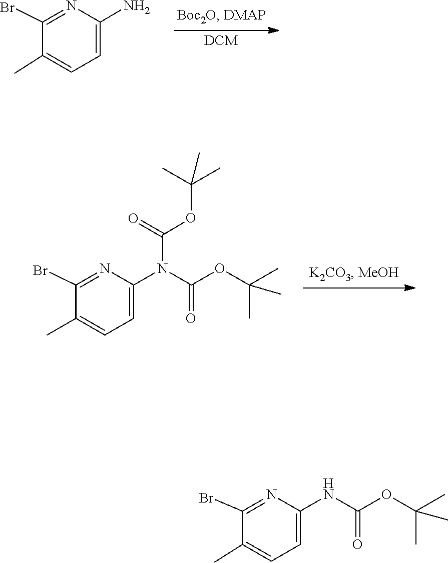

A mixture of 6-bromo-5-methylpyridin-2-amine (1000 mg, 5.35 mmol, 1.00 eq), di-tert-butyl dicarbonate (2.6 mL, 11.2 mmol, 2.10 eq), 4-dimethylaminopyridine (~5 mg) and THF (20 mL) was stirred at rt for 16 h before it was diluted with EtOAc (60 mL), washed with H₂O (50 mL), dried over MgSO₄ and filtered. The solvent was removed under reduced pressure. The crude was purified by RP chromatography (MeCN/H2O 5>95%) to afford tert-butyl N-(6-bromo-5-methyl-2-pyridyl)-N-tert-butoxycarbonyl-carbamate (315 mg, 0.813 mmol, 15%) as white crystalline solids. ¹H NMR (300 MHz, CDCl₃) δ 7.49 (dd, J=7.9, 0.8 Hz, 1H), 7.05 (d, J=7.8 Hz, 1H), 2.31 (s, 3H), 1.37 (s, 18H). MS (ES+) m/z 387.1/389.1 (M+H)⁺.

A mixture of tert-butyl N-(6-bromo-5-methyl-2-pyridyl)-N-tert-butoxycarbonyl-carbamate
(800 mg, 2.07 mmol, 1.00 eq), potassium carbonate (434 mg, 3.10 mmol, 1.50 eq) and methanol
(5 mL) was stirred at 65° C. for 1 h before it was diluted with DCM (10 mL), filtered and washed with DCM. The solvent was removed under reduced pressure and the crude was purified by RP chromatography (MeCN/H2O 5>95%) to afford tert-butyl N-(6-bromo-5-methyl-2-pyridyl)carbamate
(525 mg, 1.83 mmol, 89%) as white solids. ¹H NMR (300 MHz, CDCl₃) δ 7.78 (d, J=8.2 Hz, 1H), 7.47 (d, J=8.2 Hz, 1H), 7.24 (br, 1H), 2.31 (s, 3H), 1.50 (s, 9H). MS (ES-) m/z 285.0/287.0 (M−H)⁻.

### tert-Butyl(6-bromo-3-methylpyridin-2-yl)(tert-butoxycarbonyl)carbamate

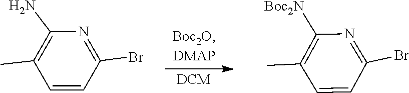

A mixture of 6-bromo-3-methylpyridin-2-amine (500 mg, 2.67 mmol, 1.00 eq), di-tert-butyl dicarbonate (1.5 mL, 6.68 mmol, 2.50 eq). 4-dimethylaminopyridine (16 mg, 0.134 mmol, 0.0500 eq) and DCM (15 mL) was stirred at 45° C. for 5 h. After cooling to rt, 2 M NaOH was added and the mixture was vigorously stirred for 5 min. It was diluted with EtOAc (50 mL), washed with H₂O (50 mL), 1 M HCl (50 mL) and sat. NaHCO₃ (50 m), dried over MgSO₄ and filtered. The solvent was removed under reduced pressure. The crude was purified by RP chromatography (MeCN/H2O 5>95%) to afford tert-butyl(6-bromo-3-methylpyridin-2-yl)(tert-butoxycarbonyl)carbamate (447 mg, 1.15 mmol, 43%) as white crystalline solids. ¹H NMR (300 MHz, CDCl₃) δ 7.45 (m, 1H), 7.37 (d, J=7.9 Hz, 1H), 2.19 (s, 3H), 1.41 (s, 18H). MS (ES+) m/z 387.2/389.2 (M+H)⁺.

### di-tert-Butyl(5-bromo-4,6-dimethylpyrimidin-2-yl)iminodicarbonate

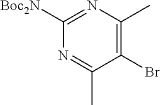

Prepared similarly from 5-bromo-4,6-dimethylpyrimidin-2-amine (600 mg, 2.97 mmol, 1.00 eq), di-tert-butyl dicarbonate (2.0 mL, 8.91 mmol, 3.00 eq), triethylamine (1.7 mL, 11.9 mmol, 4.00 eq) and DCM (8 mL) at 45° C. for 20 h to afford the title compound after RP chromatography (MeCN/H2O 5>95%) as white solids (666 mg, 1.66 mmol, 56%). ¹H NMR (300 MHz, Methanol-d4/chloroform -d) δ 2.64 (s, 6H), 1.42 (s, 18H). MS (ES+) m/z 402.5/404.5 (M+H)⁺.

### N-(6-Bromo-5-methylpyridin-2-yl)-N-ethylpivalamide

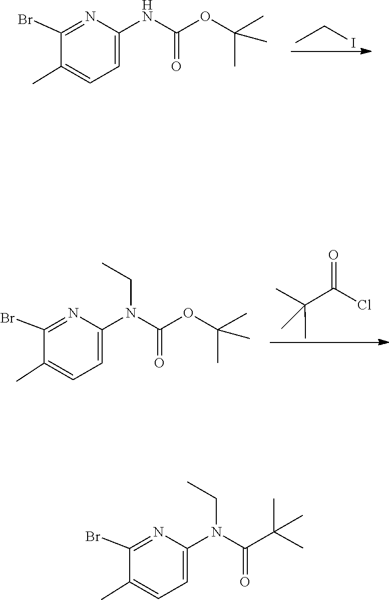

A mixture of tert-butyl N-(6-bromo-5-methyl-2-pyridyl)carbamate (200 mg, 0.696 mmol, 1.00 eq), iodoethane (0.11 mL, 1.39 mmol, 2.00 eq), potassium carbonate (195 mg, 1.39 mmol, 2.00 eq) and DMSO (2.4 mL) was stirred at rt for 48 h before the mixture was directly purified by reverse phase chromatography (MeCN/H20 5>95%) to afford tert-butyl N-(6-bromo-5-methyl-2-pyridyl)-N-ethyl-carbamate (180 mg, 0.571 mmol, 82%). ¹H NMR (300 MHz, CDCl₃) δ 7.50 (d, J=8.2 Hz, 1H), 7.40 (d, J=8.1 Hz, 1H), 3.92 (q, J=7.0 Hz, 2H), 2.29 (s, 3H), 1.49 (s, 9H), 1.19 (t, J=7.0 Hz, 3H).MS (ES+) m/z 259.2/261.2 (M-tBu+2H)+

A mixture of tert-butyl N-(6-bromo-5-methyl-2-pyridyl)-N-ethyl-carbamate (180 mg, 0.571 mmol, 1.00 eq), DCM (2 mL) and hydrochloric acid (4.0 M in dioxane, 2.0 mL) was stirred at rt for 3 h before it was basified with 1 M NaOH(15 mL) and extracted with EtOAc (3*15 mL). The combined organic phases were dried over MgSO₄, filtered and the solvent was removed under reduced pressure. DCM (3 mL) was added, followed by trimethylacetyl chloride (0.21 mL, 1.71 mmol, 3.00 eq) and pyridine (0.18 mL, 2.28 mmol, 4.00 eq) and the mixture was stirred at 40° C. for 16 h. After cooling to rt, the mixture was diluted with EtOAc (15 mL), washed with 0.5 M HCl (10 m), sat. NaHCO₃ (10 mL), dried over MgSO₄ and filtered. The solvent was removed under reduced pressure and the crude was purified by RP chromatography (MeCN/H20 5>95%) to afford N-(6-bromo-5-methyl-2-pyridyl)-N-ethyl-2,2-dimethyl-propanamide (84 mg, 0.281 mmol, 49%) as white crystals. ¹H NMR (300 MHz, CDCl₃) δ 7.61-7.50 (m, 1H), 7.06 (d, J=7.8 Hz, 1H), 3.74 (q, J=7.1 Hz, 2H), 2.42 (s, 3H), 1.11 (t, J=7.1 Hz, 3H), 1.06 (s, 9H).MS (ES+) m/z 299.0/301.0 (M+H)+

### Syntheses from Unprotected Phenol. Pd Coupling of 2-bromo-N-(3-hydroxy-2,6-disubstituted-phenyl)thiazole-5-carboxamide with substituted amines

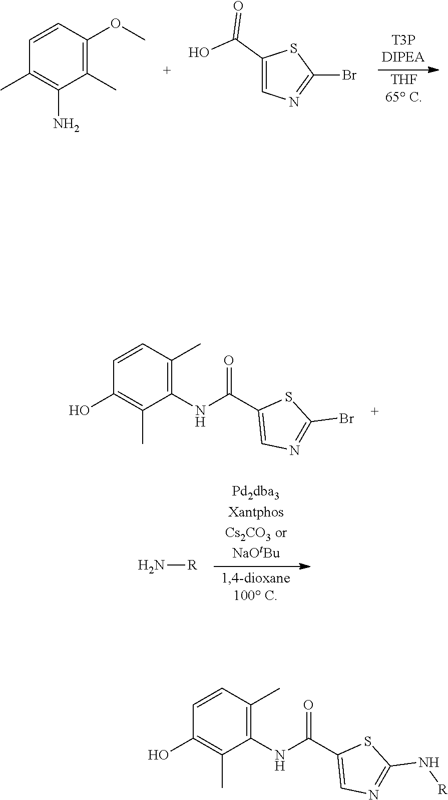

### 2-Bromo-N-(3-hydroxy-2,6-dimethyl-phenyl)thiazole-5-carboxamide

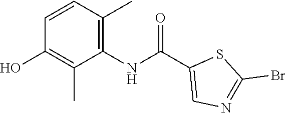 [SMILES: Cc1ccc(O)c(C)c1NC(=O)c1cnc(Br)s1]

To a stirring solution of 2-bromo-1,3-thiazole-5-carboxylic acid (300 mg, 1.44 mmol, 1.0 eq) and 3-amino-2,4-dimethyl-phenol (237 mg, 1.73 mmol, 1.2 eq) in THF (10 mL) was added DIPEA
(0.50 mL, 2.88 mmol, 2.0 eq) and T3P (50% in EtOAc, 1.3 mL, 2.16 mmol, 1.5 eq). The reaction mixture was heated to 65° C. and left to stir overnight. The mixture was allowed to cool to RT, water and EtOAc added, and the layers separated. The aqueous layer was extracted further with EtOAc, organics collected, passed through a phase separator and the solvents removed in vacuo. The crude material was chromatographed (SiO₂) eluting with 0-100% EtOAc: PE to afford the title compound (288 mg, 0.880 mmol, 61%) as a thick colourless oil. MS (ES+) m/z 327.1/329.1 (M+H), Br isotope pattern. ¹H NMR, (300 MHz, DMSO-d₆) δ 10.04 (s, 1H), 9.25 (s, 1H), 8.42 (s, 1H), 6.91 (d, J=8.2 Hz, 1H), 6.72 (d, J=8.2 Hz, 1H), 2.06 (s, 3H), 1.98 (s, 3H).

General Method C: Palladium catalysed coupling of 2-bromo-N-(3-hydroxy-2,6-dimethyl-phenyl)thiazole-5-carboxamide with amines in anhydrous solvents

To a mixture of 2-bromo-N-(3-hydroxy-2,6-dimethyl-phenyl)thiazole-5-carboxamide (1 eq), substituted amino pyrazole (1.2-2 eq) and Cs₂CO₃ or sodium tert-butoxide (2-5.5 eq) in 1,4-dioxane (0.02-0.1 M) was flushed with nitrogen before the addition of Pd₂dba₃ (0.05-0.1 eq) and XantPhos (0.1 eq). The mixture was heated to 100° C. for 18 h. The progress of the reaction was monitored by consumption of starting material via UPLC. The mixture was cooled and concentrated in vacuo. The crude material was purified by either normal phase chromatography (SiO₂) using a gradient of EtOAc: PE and/or MeOH: DCM, reverse phase chromatography (C18) using a gradient of MeCN: H₂O and/or by preparative HPLC-MS using a gradient of high or low pH aq. MeCN.

### N-(3-Hydroxy-2,6-dimethyl-phenyl)-2-[[1-(3-hydroxypropyl)pyrazol-3-yl]amino]thiazole-5-carboxamide (BAA-002)

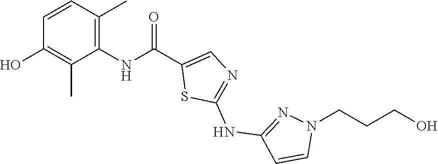 [SMILES: Cc1ccc(O)c(C)c1NC(=O)c1cnc(Nc2ccn(CCCO)n2)s1]

Prepared as described in Method C from 2-bromo-N-(3-hydroxy-2,6-dimethyl-phenyl)thiazole-5-carboxamide (300 mg, 0.917 mmol, 1.0 eq), 3-(3-aminopyrazol-1-yl)propan-1-ol
(194 mg, 1.38 mmol, 1.5 eq), Cs₂CO₃ (751 mg, 2.29 mmol, 2.5 eq), Pd₂dba₃ (84 mg, 0.0917 mmol, 0.1 eq) and XantPhos (53 mg, 0.0917 mmol, 0.1 eq) in 1,4-dioxane (40 mL) to return the title compound (190 mg, 0.490 mmol, 53%) as an orange oil after normal phase chromatography (SiO₂) eluting with 0-30% MeOH: DCM and subsequent reverse phase chromatography (C18) using 5-95% MeCN: H2O as eluent. MS (ES+) m/z 388.4 (M+H). ¹H NMR (300 MHz, Methanol-d4) δ 8.05 (s, 1H), 7.55 (d, J=2.4 Hz, 1H), 6.93 (d, J=8.2 Hz, 1H), 6.70 (d, J=8.2 Hz, 1H), 6.03 (d, J=2.3 Hz, 1H), 4.21 (t, J=6.8 Hz, 2H), 3.59 (t, J=6.2 Hz, 2H), 2.18 (s, 3H), 2.14-2.04 (m, 5H).

The following example compounds were prepared similarly using Method C using the appropriately substituted amine.

| # | Structure & Name | Amine | Analytical Data |
|---|---|---|---|
| BAA- 003 | 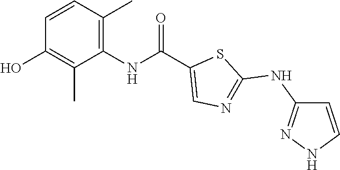 [SMILES: Cc1ccc(O)c(C)c1NC(=O)c1cnc(Nc2cc[nH]n2)s1] | 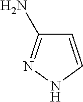 | MS (ES+) m/z 330.1 (M + H) ¹H NMR, (300 MHz, DMSO-d₆) δ 9.80 (s, 1H), 9.26 (br s, 1H), 8.28 (s, 1H), 8.17 (d, J = 2.8 Hz, 1H), 6.90 (d, J = 8.2 Hz, 1H), 6.71 (d, J = 8.2 Hz, 1H), 5.93 (d, J = 2.8 Hz, 1H), 5.74 (s, 2H), 2.08 (s, 3H), 2.00 (s, 3H). |
| | N-(3-hydroxy-2,6-dimethyl- | | |
| | phenyl)-2-(1H-pyrazol-3- | | |
| | ylamino)thiazole-5- | | |
| | carboxamide | | |
| BAA- 004 | 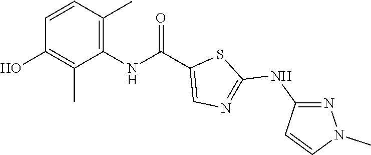 [SMILES: Cc1ccc(O)c(C)c1NC(=O)c1cnc(Nc2ccn(C)n2)s1] | 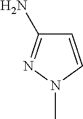 | MS (ES+) m/z 344.3 (M + H). ¹H NMR (300 MHz, Methanol-d₄) δ 8.02 (s, 1H), 7.48 (d, J = 2.3 Hz, 1H), 6.91 (d, J = 8.2 Hz, 1H), 6.68 (d, J = 8.2 Hz, 1H), 6.03 (d, J = 2.3 Hz, 1H), 3.84 (s, 3H), 2.16 (s, 3H), 2.11 (s, 3H). |
| | N-(3-hydroxy-2,6-dimethyl- | | |
| | phenyl)-2-[(1-methylpyrazol-3- | | |
| | yl)amino]thiazole-5- | | |
| | carboxamide | | |
| BAA- 005 | 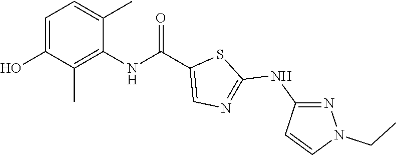 [SMILES: CCn1ccc(Nc2ncc(C(=O)Nc3c(C)ccc(O)c3C)s2)n1] | 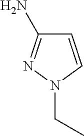 | MS (ES+) m/z 358.3 (M + H). ¹H NMR (300 MHz, Methanol-d₄) δ− 8.03 (s, 1H), 7.52 (d, J = 2.3 Hz, 1H), 6.91 (d, J = 8.3 Hz, 1H), 6.68 (d, J = 8.2 Hz, 1H), 6.01 (d, J = 2.4 Hz, 1H), 4.12 (q, J = 7.2 Hz, 2H), 2.16 (s, 3H), 2.12 (s, 3H), 1.47 (t, J = 7.3 Hz, 3H). |
| | 2-[(1-ethylpyrazol-3-yl)amino]- | | |
| | N-(3-hydroxy-2,6-dimethyl- | | |
| | phenyl)thiazole-5-carboxamide | | |
| BAA- 006 | 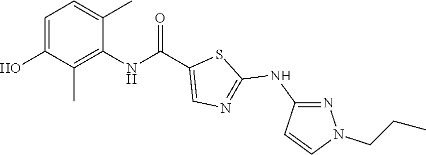 [SMILES: CCCn1ccc(Nc2ncc(C(=O)Nc3c(C)ccc(O)c3C)s2)n1] | 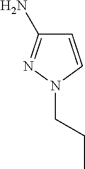 | MS (ES+) m/z 372.3 (M + H). ¹H NMR (300 MHz, Methanol-d₄) δ 8.03 (s, 1H), 7.51 (d, J = 2.3 Hz, 1H), 6.91 (d, J = 8.2 Hz, 1H), 6.68 (d, J = 8.2 Hz, 1H), 6.01 (d, J = 2.3 Hz, 1H), 4.04 (t, J = 6.9 Hz, 2H), 2.16 (s, 3H), 2.12 (s, 3H), 1.91 (q, J = 7.1 Hz, 2H), 0.92 (t, J = 7.4 Hz, 3H). |
| | N-(3-hydroxy-2,6-dimethyl- | | |
| | phenyl)-2-[(1-propylpyrazol-3- | | |
| | yl) amino]thiazole-5- | | |
| | carboxamide | | |
| BAA- 007 | 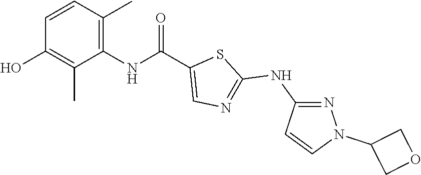 [SMILES: Cc1ccc(O)c(C)c1NC(=O)c1cnc(Nc2ccn(C3COC3)n2)s1] | 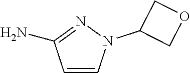 | MS (ES+) m/z 386.3 (M + H). ¹H NMR (300 MHz, Methanol-d₄) δ 8.06 (s, 1H), 7.62 (d, J = 2.4 Hz, 1H), 6.92 (d, J = 8.3 Hz, 1H), 6.69 (d, J = 8.2 Hz, 1H), 6.05 (d, J = 2.4 Hz, 1H), 5.51 (tt, J = 7.5, 6.4 Hz, 1H), 5.18 (t, J = 6.5 Hz, 2H), 5.04- 4.97 (m, 2H), 2.17 (s, 3H), 2.12 (s, 3H). |
| | N-(3-hydroxy-2,6-dimethyl- | | |
| | phenyl)-2-[[1-(oxetan-3- | | |
| | yl)pyrazol-3-yl]amino]thiazole- | | |
| | 5-carboxamide | | |
| BAA- 008 | 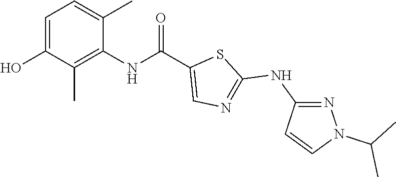 [SMILES: Cc1ccc(O)c(C)c1NC(=O)c1cnc(Nc2ccn(C(C)C)n2)s1] | 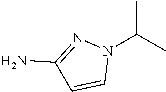 | MS (ES+) m/z 372.3 (M + H). ¹H NMR (300 MHz, Methanol-d₄) δ 8.03 (s, 1H), 7.54 (d, J = 2.4 Hz, 1H), 6.91 (d, J = 8.2 Hz, 1H), 6.68 (d, J = 8.2 Hz, 1H), 5.98 (d, J = 2.4 Hz, 1H), 4.44 (p, J = 6.6 Hz, 1H), 2.17 (s, 3H), 2.12 (s, 3H), 1.51 (d, J = 6.6 Hz, 6H). |
| | N-(3-hydroxy-2,6-dimethyl- | | |
| | phenyl)-2-[(1-isopropylpyrazol- | | |
| | 3-yl)amino]thiazole-5- | | |
| | carboxamide | | |
| BAA- 009 | 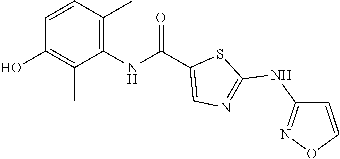 [SMILES: Cc1ccc(O)c(C)c1NC(=O)c1cnc(Nc2ccon2)s1] | 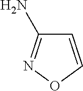 | MS (ES+) m/z 331.2 (M + H). ¹H NMR (300 MHz, Methanol-d₄) δ 8.52 (d, J = 1.8 Hz, 1H), 8.09 (s, 1H), 6.92 (d, J = 8.2 Hz, 1H), 6.69 (d, J = 8.2 Hz, 1H), 6.42 (d, J = 1.8 Hz, 1H), 2.16 (s, 3H), 2.12 (s, 3H). |
| | N-(3-hydroxy-2,6-dimethyl- | | |
| | phenyl)-2-(isoxazol-3- | | |
| | ylamino)thiazole-5- | | |
| | carboxamide | | |
| BAA- 010 | 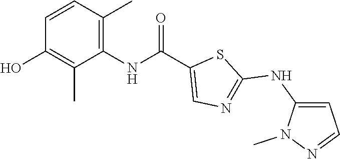 [SMILES: Cc1ccc(O)c(C)c1NC(=O)c1cnc(Nc2ccnn2C)s1] | 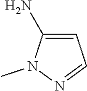 | MS (ES+) m/z 344.3 (M + H). ¹H NMR (300 MHz, Methanol-d₄) δ 7.95 (s, 1H), 7.46 (d, J = 2.1 Hz, 1H), 6.91 (d, J = 8.3 Hz, 1H), 6.68 (d, J = 8.2 Hz, 1H), 6.35 (d, J = 2.0 Hz, 1H), 3.78 (s, 3H), 2.15 (s, 3H), 2.10 (s, 3H). |
| | N-(3-hydroxy-2,6-dimethyl- | | |
| | phenyl)-2-[(2-methylpyrazol-3- | | |
| | yl) amino]thiazole-5- | | |
| | carboxamide | | |
| BAA- 011 | 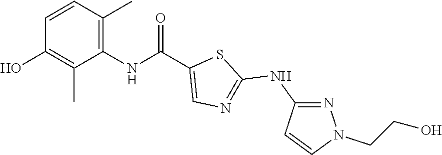 [SMILES: Cc1ccc(O)c(C)c1NC(=O)c1cnc(Nc2ccn(CCO)n2)s1] | 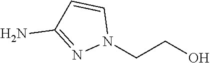 | MS (ES+) m/z 374.3 (M + H). ¹H NMR (300 MHz, Methanol-d₄) δ 8.03 (s, 1H), 7.55 (d, J = 2.4 Hz, 1H), 6.91 (d, J = 8.2 Hz, 1H), 6.68 (d, J = 8.2 Hz, 1H), 6.02 (d, J = 2.4 Hz, 1H), 4.17 (t, J = 5.4 Hz, 2H), 3.94 (t, J = 5.4 Hz, 2H), 2.16 (s, 3H), 2.11 (s, 3H). |
| | N-(3-hydroxy-2,6-dimethyl- | | |
| | phenyl)-2-[1-(2- | | |
| | hydroxyethyl)pyrazol-3- | | |
| | yl]amino]thiazole-5- | | |
| | carboxamide | | |
| BAA- 012 | 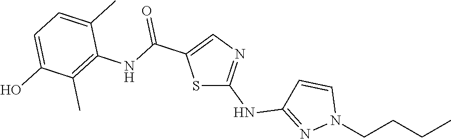 [SMILES: CCCCn1ccc(Nc2ncc(C(=O)Nc3c(C)ccc(O)c3C)s2)n1] | 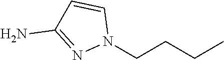 | MS (ES+) m/z 386.3 (M + H). ¹H NMR (300 MHz, Methanol-d₄) δ 8.03 (s, 1H), 7.51 (d, J = 2.3 Hz, 1H), 6.91 (d, J = 8.2 Hz, 1H), 6.68 (d, J = 8.2 Hz, 1H), 6.00 (d, J = 2.3 Hz, 1H), 4.08 (t, J = 6.9 Hz, 2H), 2.16 (s, 3H), 2.12 (s, 3H), 1.87 (p, J = 7.1 Hz, 2H), 1.33 (dt, J = 14.6, 7.4 Hz, 2H), 0.96 (t, J = 7.4 Hz, 3H). |
| | 2-[(1-butylpyrazol-3-yl)amino]- | | |
| | N-(3-hydroxy-2,6-dimethyl- | | |
| | phenyl)thiazole-5-carboxamide | | |
| BAA- 013 | 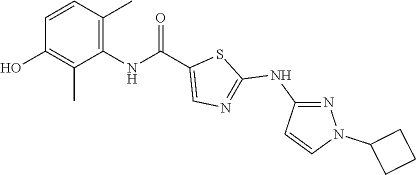 [SMILES: Cc1ccc(O)c(C)c1NC(=O)c1cnc(Nc2ccn(C3CCC3)n2)s1] | 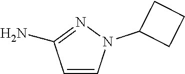 | MS (ES+) m/z 384.3 (M + H). ¹H NMR (300 MHz, Methanol-d₄) δ 8.05 (s, 1H), 7.54 (d, J = 2.4 Hz, 1H), 6.92 (d, J = 8.2 Hz, 1H), 6.69 (d, J = 8.2 Hz, 1H), 5.98 (d, J = 2.4 Hz, 1H), 4.79-4.68 (m, 1H), 2.65 (pd, J = 9.3, 2.7 Hz, 2H), 2.47-2.36 (m, 2H), 2.17 (s, 3H), 2.12 (s, 3H), 1.94-1.78 (m, 2H). |
| | 2-[(1-cyclobutylpyrazol-3- | | |
| | yl)amino]-N-(3-hydroxy-2,6- | | |
| | dimethyl-phenyl)thiazole-5- | | |
| | carboxamide | | |
| BAA- 014 | 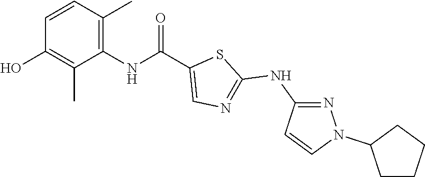 [SMILES: Cc1ccc(O)c(C)c1NC(=O)c1cnc(Nc2ccn(C3CCCC3)n2)s1] | 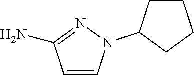 [SMILES: Nc1ccn(C2CCCC2)n1] | MS (ES+) m/z 398.3 (M + H). ¹H NMR (300 MHz, Methanol-d₄) δ 8.04 (s, 1H), 7.53 (d, J = 2.4 Hz, 1H), 6.91 (d, J = 8.2 Hz, 1H), 6.68 (d, J = 8.2 Hz, 1H), 5.96 (d, J = 2.4 Hz, 1H), 4.63 (p, J = 6.8 Hz, 1H), 2.17 (s, 3H), 2.14-2.06 (m, 7H), 1.98-1.87 (m, 2H), 1.71 (ttt, J = 8.0, 5.6, 2.2 Hz, 2H). |
| | 2-[(1-cyclopentylpyrazol-3- | | |
| | yl)amino]-N-(3-hydroxy-2,6- | | |
| | dimethyl-phenyl)thiazole-5- | | |
| | carboxamide | | |
| BAA- 015 | 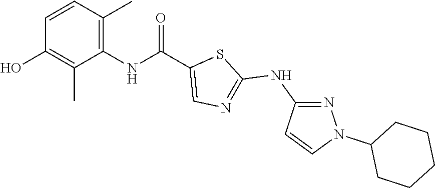 [SMILES: Cc1ccc(O)c(C)c1NC(=O)c1cnc(Nc2ccn(C3CCCCC3)n2)s1] | 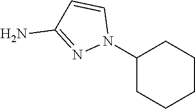 [SMILES: Nc1ccn(C2CCCCC2)n1] | MS (ES+) m/z 412.3 (M + H). ¹H NMR (300 MHz, DMSO-d₆) δ 11.00 (s, 1H), 9.47 (s, 1H), 9.15 (s, 1H), 8.09 (s, 1H), 7.66 (s, 1H), 6.88 (d, J = 8.2 Hz, 1H), 6.68 (d, J = 8.2 Hz, 1H), 5.92 (s, 1H), 4.04 (tt, J = 11.0, 3.7 Hz, 1H), 2.03 (d, J = 24.3 Hz, 8H), 1.87-1.60 (m, 5H), 1.48- 1.12 (m, 3H). |
| | 2-[(1-cyclohexylpyrazol-3- | | |
| | yl)amino]-N-(3-hydroxy-2,6- | | |
| | dimethyl-phenyl)thiazole-5- | | |
| | carboxamide | | |
| BAA- 016 | 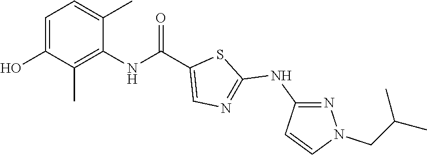 [SMILES: Cc1ccc(O)c(C)c1NC(=O)c1cnc(Nc2ccn(CC(C)C)n2)s1] | 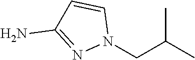 | MS (ES+) m/z 386.0 (M + H). ¹H NMR (300 MHz, DMSO-d₆) δ 11.00 (s, 1H), 9.47 (s, 1H), 9.15 (s, 1H), 8.09 (s, 1H), 7.63 (d, J = 2.3 Hz, 1H), 6.88 (d, J = 8.2 Hz, 1H), 6.68 (d, J = 8.2 Hz, 1H), 5.93 (d, J = 2.3 Hz, 1H), 3.84 (d, J = 7.1 Hz, 2H), 2.16 (dt, J = 13.5, 6.7 Hz, 1H), 2.07 (s, 3H), 1.98 (s, 3H), 0.85 (d, J = 6.7 Hz, 6H). |
| | N-(3-hydroxy-2,6-dimethyl- | | |
| | phenyl)-2-[(1-isobutylpyrazol-3- | | |
| | yl)amino]thiazole-5- | | |
| | carboxamide | | |
| BAA- 017 | 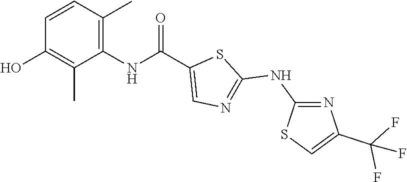 [SMILES: Cc1ccc(O)c(C)c1NC(=O)c1cnc(Nc2nc(C(F)(F)F)cs2)s1] | 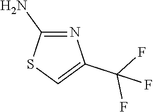 | MS (ES+) m/z 415.3 (M + H). ¹H NMR (300 MHz, DMSO-d₆) δ 12.86 (s, 1H), 9.72 (s, 1H), 9.20 (s, 1H), 8.22 (s, 1H), 7.89 (s, 1H), 6.90 (d, J = 8.2 Hz, 1H), 6.70 (d, J = 8.2 Hz, 1H), 2.07 (s, 3H), 1.99 (s, 3H). ¹⁹F NMR (282 MHz, DMSO-d⁶) δ −63.34. |
| | N-(3-hydroxy-2,6-dimethyl- | | |
| | phenyl)-2-[4- | | |
| | (trifluoromethyl)thiazol-2- | | |
| | yl]amino]thiazole-5- | | |
| | carboxamide | | |
| BAA- 018 | 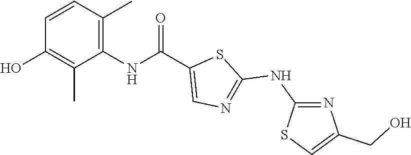 [SMILES: Cc1ccc(O)c(C)c1NC(=O)c1cnc(Nc2nc(CO)cs2)s1] | 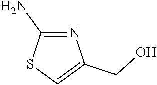 | MS (ES+) m/z 377.3 (M + H). ¹H NMR (300 MHz, Methanol-d₄) δ 8.10 (s, 1H), 6.92 (d, J = 8.2 Hz, 1H), 6.84 (s, 1H), 6.69 (d, J = 8.2 Hz, 1H), 4.60 (d, J = 1.0 Hz, 2H), 2.16 (s, 3H), 2.11 (s, 3H). |
| | N-(3-hydroxy-2,6-dimethyl- | | |
| | phenyl)-2-[4- | | |
| | (hydroxymethyl)thiazol-2- | | |
| | yl]amino]thiazole-5- | | |
| | carboxamide | | |
| BAA- 019 | 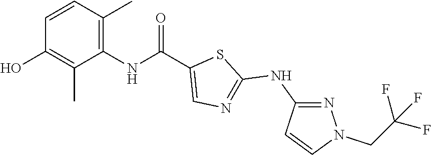 [SMILES: Cc1ccc(O)c(C)c1NC(=O)c1cnc(Nc2ccn(CC(F)(F)F)n2)s1] | 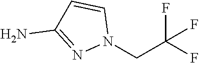 [SMILES: Nc1ccn(CC(F)(F)F)n1] | MS (ES+) m/z 411.9 (M + H). ¹H NMR (300 MHz, Methanol-d₄) δ 8.05 (s, 1H), 7.66 (d, J = 2.5 Hz, 1H), 6.91 (d, J = 8.2 Hz, 1H), 6.69 (d, J = 8.2 Hz, 1H), 6.15 (d, J = 2.5 Hz, 1H), 4.86 (q, J = 8.7 Hz, 2H), 2.16 (s, 3H), 2.12 (s, 3H). |
| | N-(3-hydroxy-2,6-dimethyl- | | |
| | phenyl)-2-[[1-(2,2,2- | | |
| | trifluoroethyl)pyrazol-3- | | |
| | yl]amino]thiazole-5- | | |
| | carboxamide | | |
| BAA- 020 | 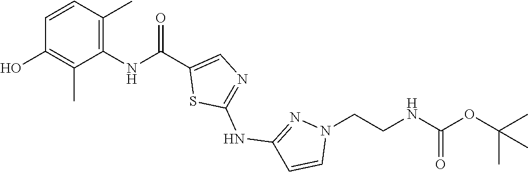 [SMILES: Cc1ccc(O)c(C)c1NC(=O)c1cnc(Nc2ccn(CCNC(=O)OC(C)(C)C)n2)s1] | 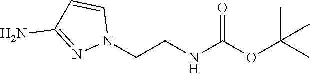 | MS (ES+) m/z 473.0 (M + H). ¹H NMR (300 MHz, Methanol-d₄) δ 8.03 (s, 1H), 7.49 (d, J = 2.4 Hz, 1H), 6.91 (d, J = 8.2 Hz, 1H), 6.68 (d, J = 8.2 Hz, 1H), 6.02 (d, J = 2.4 Hz, 1H), 4.15 (t, J = 6.0 Hz, 2H), 3.48 (t, J = 6.0 Hz, 2H), 2.16 (s, 3H), 2.12 (s, 3H), 1.40 (s, 9H). |
| | tert-butyl N-[2-[3-[5-[(3- | | |
| | hydroxy-2,6-dimethyl- | | |
| | phenyl)carbamoyl]thiazol-2- | | |
| | yl]amino]pyrazol-1- | | |
| | ylethyl]carbamate | | |
| BAA- 021 | 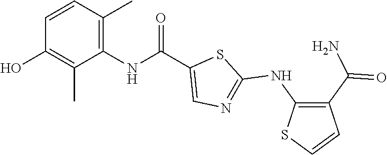 [SMILES: Cc1ccc(O)c(C)c1NC(=O)c1cnc(Nc2sccc2C(N)=O)s1] | 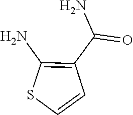 | MS (ES+) m/z 388.9 (M + H). ¹H NMR (300 MHz, Methanol-d₄) δ 8.10 (s, 1H), 7.31 (d, J = 5.9 Hz, 1H), 6.92 (d, J = 8.2 Hz, 1H), 6.85 (d, J = 5.8 Hz, 1H), 6.69 (d, J = 8.2 Hz, 1H), 2.16 (s, 3H), 2.11 (s, 3H). |
| | 2-[(3-carbamoyl-2- | | |
| | thienyl)amino]-N-(3-hydroxy- | | |
| | 2,6-dimethyl-phenyl)thiazole-5- | | |
| | carboxamide | | |
| BAA- 022 | 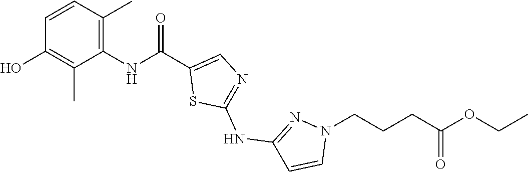 [SMILES: CCOC(=O)CCCn1ccc(Nc2ncc(C(=O)Nc3c(C)ccc(O)c3C)s2)n1] | 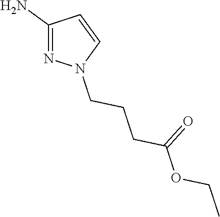 | MS (ES+) m/z 443.9 (M + H). ¹H NMR (300 MHz, Methanol-d₄) δ 8.05 (s, 1H), 7.53 (d, J = 2.4 Hz, 1H), 7.01-6.87 (m, 1H), 6.70 (d, J = 8.2 Hz, 1H), 6.03 (d, J = 2.4 Hz, 1H), 4.23-4.01 (m, 4H), 2.35 (td, J = 7.0, 1.1 Hz, 2H), 2.26-2.07 (m, 8H), 1.24 (t, J = 7.1 Hz, 3H). |
| | ethyl 4-[3-[5-[(3-hydroxy-2,6- | | |
| | dimethyl- | | |
| | phenyl)carbamoyl]thiazol-2- | | |
| | yl]amino]pyrazol-1-yl]butanoate | | |
| BAA- 023 | 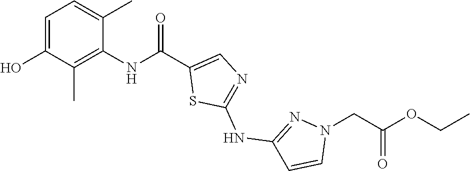 [SMILES: CCOC(=O)Cn1ccc(Nc2ncc(C(=O)Nc3c(C)ccc(O)c3C)s2)n1] | 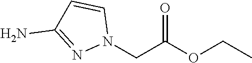 | MS (ES+) m/z 415.9 (M + H). ¹H NMR (300 MHz, Methanol-d₄) δ 8.05 (s, 1H), 7.60 (d, J = 2.5 Hz, 1H), 6.98-6.87 (m, 1H), 6.70 (d, J = 8.2 Hz, 1H), 6.12 (d, J = 2.4 Hz, 1H), 4.95 (s, 2H), 4.24 (q, J = 7.1 Hz, 2H), 2.18 (s, 3H), 2.13 (s, 3H), 1.29 (t, J = 7.1 Hz, 3H). |
| | ethyl 2-[3-[5-[(3-hydroxy-2,6- | | |
| | dimethyl- | | |
| | phenyl)carbamoyl]thiazol-2- | | |
| | yl]amino]pyrazol-1-yl]acetate | | |
| BAA- 024 | 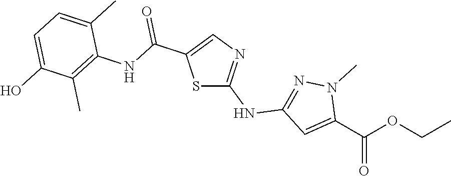 [SMILES: CCOC(=O)c1cc(Nc2ncc(C(=O)Nc3c(C)ccc(O)c3C)s2)nn1C] | 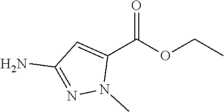 | MS (ES+) m/z 415.9 (M + H). ¹H NMR (300 MHz, Methanol-d₄) δ 8.04 (s, 1H), 6.92 (d, J = 8.2 Hz, 1H), 6.69 (d, J = 8.2 Hz, 1H), 6.64 (s, 1H), 4.36 (q, J = 7.1 Hz, 2H), 4.12 (s, 3H), 2.16 (s, 3H), 2.12 (s, 3H), 1.38 (t, J = 7.1 Hz, 3H). |
| | ethyl 5-[5-[(3-hydroxy-2,6- | | |
| | dimethyl- | | |
| | phenyl)carbamoyl]thiazol-2- | | |
| | yl]amino]-2-methyl-pyrazole-3- | | |
| | carboxylate | | |
| BAA- 025 | 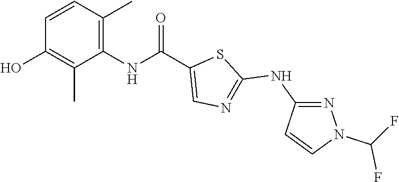 [SMILES: Cc1ccc(O)c(C)c1NC(=O)c1cnc(Nc2ccn(C(F)F)n2)s1] | 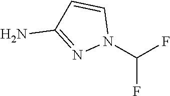 | MS (ES+) m/z 379.9 (M + H). ¹H NMR (300 MHz, Methanol-d₄) δ 8.07 (s, 1H), 7.97 (d, J = 2.8 Hz, 1H), 7.40 (t, J = 60.0 Hz, 1H), 6.92 (d, J = 8.2 Hz, 1H), 6.72-6.65 (m, 1H), 6.30 (d, J = 2.8 Hz, 1H), 2.17 (s, 3H), 2.12 (s, 3H). ¹⁹F NMR (282 MHz, Methanol-d₄) δ −95.96, −96.17. |
| | 2-[[1-(difluoromethyl)pyrazol-3- | | |
| | yl]amino]-N-(3-hydroxy-2,6- | | |
| | dimethyl-phenyl)thiazole-5- | | |
| | carboxamide | | |
| BAA- 026 | 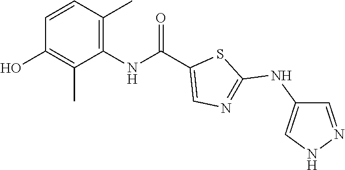 [SMILES: Cc1ccc(O)c(C)c1NC(=O)c1cnc(Nc2cn[nH]c2)s1] | 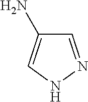 | MS (ES+) m/z 330.3 (M + H). ¹H NMR (300 MHz, Methanol-d₄) δ 8.21 (s, 1H), 7.85 (d, J = 0.9 Hz, 1H), 7.49 (d, J = 0.9 Hz, 1H), 6.93 (d, J = 8.3 Hz, 1H), 6.70 (d, J = 8.2 Hz, 1H), 2.17 (s, 3H), 2.11 (s, 3H). |
| | N-(3-hydroxy-2,6-dimethyl- | | |
| | phenyl)-2-(1H-pyrazol-4- | | |
| | ylamino)thiazole-5- | | |
| | carboxamide | | |
| BAA- 027 | 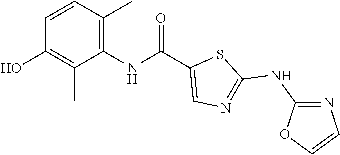 [SMILES: Cc1ccc(O)c(C)c1NC(=O)c1cnc(Nc2ncco2)s1] | 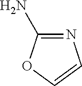 | MS (ES+) m/z 330.9 (M + H). ¹H NMR (300 MHz, Methanol-d₄) δ 8.03 (s, 1H), 7.53 (d, J = 0.9 Hz, 1H), 7.08 (d, J = 0.9 Hz, 1H), 6.92 (d, J = 8.2 Hz, 1H), 6.69 (d, J = 8.2 Hz, 1H), 2.16 (s, 3H), 2.11 (s, 3H). |
| | N-(3-hydroxy-2,6-dimethyl- | | |
| | phenyl)-2-(oxazol-2- | | |
| | ylamino)thiazole-5- | | |
| | carboxamide | | |
| BAA- 028 | 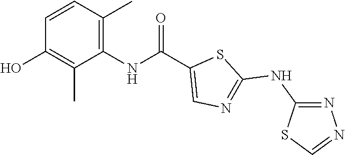 [SMILES: Cc1ccc(O)c(C)c1NC(=O)c1cnc(Nc2nncs2)s1] | 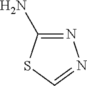 | MS (ES+) m/z 347.8 (M + H). ¹H NMR (300 MHz, Methanol-d₄) δ 8.88 (s, 1H), 8.12 (s, 1H), 6.92 (d, J = 8.2 Hz, 1H), 6.69 (d, J = 8.2 Hz, 1H), 2.16 (s, 3H), 2.11 (s, 3H). |
| | N-(3-hydroxy-2,6-dimethyl- | | |
| | phenyl)-2-(1,3,4-thiadiazol-2- | | |
| | ylamino)thiazole-5- | | |
| | carboxamide | | |
| BAA- 029 | 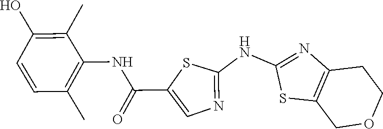 [SMILES: Cc1ccc(O)c(C)c1NC(=O)c1cnc(Nc2nc3c(s2)COCC3)s1] | 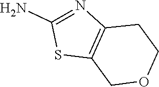 | MS (ES+) m/z 402.9 (M + H). ¹H NMR (300 MHz, DMSO-d₆) δ 12.42 (s, 1H), 9.63 (s, 1H), 9.21 (s, 1H), 8.17 (s, 1H), 6.89 (d, J = 8.2 Hz, 1H), 6.69 (d, J = 8.2 Hz, 1H), 4.64 (s, 2H), 3.92 (t, J = 5.4 Hz, 2H), 2.68 (s, 2H), 2.06 (s, 3H), 1.98 (s, 3H). |
| | 2-(6,7-dihydro-4H-pyrano[4,3- | | |
| | d]thiazol-2-ylamino)-N-(3- | | |
| | hydroxy-2,6-dimethyl- | | |
| | phenyl)thiazole-5-carboxamide | | |
| BAA- 101 | 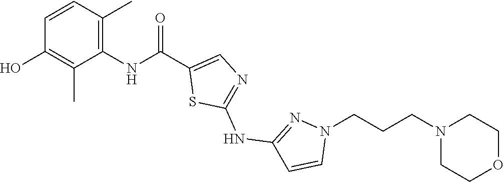 [SMILES: Cc1ccc(O)c(C)c1NC(=O)c1cnc(Nc2ccn(CCCN3CCOCC3)n2)s1] | 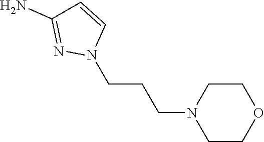 [SMILES: Nc1ccn(CCCN2CCOCC2)n1] | MS (ES+) m/z 457.3 (M + H). ¹H NMR (300 MHz, Methanol-d₄) δ 7.99 (s, 1H), 7.52 (d, J = 2.1 Hz, 1H), 6.91 (d, J = 8.2 Hz, 1H), 6.68 (d, J = 8.2 Hz, 1H), 6.37 (d, J = 2.1 Hz, 1H), 4.18 (t, J = 6.4 Hz, 2H), 3.86-3.73 (m, 4H), 2.44 (s, 4H), 2.27 (t, J = 6.8 Hz, 2H), 2.14 (s, 3H), 2.10 (s, 3H), 2.07-2.01 (m, 2H). |
| | N-(3-hydroxy-2,6-dimethyl- | | |
| | phenyl)-2-[[1-(3- | | |
| | morpholinopropyl)pyrazol-3- | | |
| | ylamino]thiazole-5- | | |
| | carboxamide | | |
| BAA- 030 | 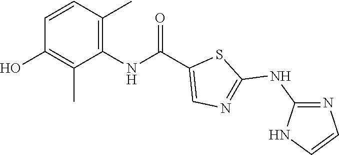 [SMILES: Cc1ccc(O)c(C)c1NC(=O)c1cnc(Nc2ncc[nH]2)s1] | 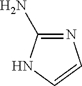 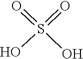 | MS (ES+) m/z 329.9 (M + H). ¹H NMR (300 MHz, Methanol-d₄) δ− 8.30 (s, 1H), 7.08 (d, J = 2.0 Hz, 1H), 6.94 (d, J = 8.2 Hz, 1H), 6.71 (d, J = 8.2 Hz, 1H), 6.68 (d, J = 2.0 Hz, 1H), 2.17 (s, 3H), 2.12 (s, 3H). |
| | N-(3-hydroxy-2,6-dimethyl- | | |
| | phenyl)-2-(1H-imidazol-2- | | |
| | ylamino)thiazole-5- | | |
| | carboxamide | | |
| BAA- 031 | 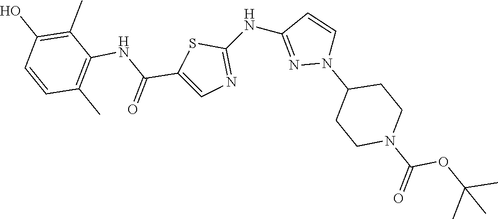 [SMILES: Cc1ccc(O)c(C)c1NC(=O)c1cnc(Nc2ccn(C3CCN(C(=O)OC(C)(C)C)CC3)n2)s1] | 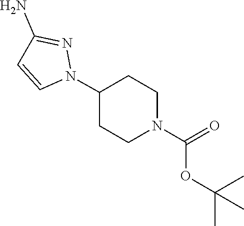 | MS (ES+) m/z 513.4 (M + H). ¹H NMR (300 MHz, Methanol-d₄) δ 7.96 (s, 1H), 7.53 (d, J = 2.1 Hz, 1H), 6.92 (d, J = 8.3 Hz, 1H), 6.70 (d, J = 8.2 Hz, 1H), 6.34 (d, J = 2.1 Hz, 1H), 4.52 (td, J = 6.9, 3.4 Hz, 1H), 4.24 (d, J = 13.3 Hz, 2H), 2.91 (s, 2H), 2.16 (s, 3H), 2.11 (s, 3H), 2.09-1.85 (m, 4H), 1.50 (s, 9H). |
| | tert-butyl 4-[3-[5-[(3-hydroxy- | | |
| | 2,6-dimethyl- | | |
| | phenyl)carbamoyl]thiazol-2- | | |
| | yl]amino]pyrazol-1- | | |
| | yl]piperidine-1-carboxylate | | |
| BAA- 032 | 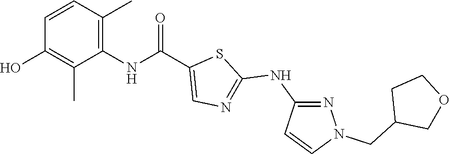 [SMILES: Cc1ccc(O)c(C)c1NC(=O)c1cnc(Nc2ccn(CC3CCOC3)n2)s1] | 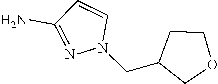 [SMILES: Nc1ccn(CC2CCOC2)n1] | MS (ES+) m/z 414.0 (M + H). ¹H NMR (300 MHz, Methanol-d₄) δ 8.05 (s, 1H), 7.55 (d, J = 2.4 Hz, 1H), 6.91 (d, J = 8.2 Hz, 1H), 6.68 (d, J = 8.2 Hz, 1H), 6.00 (d, J = 2.4 Hz, 1H), 4.17-3.99 (m, 2H), 3.92- 3.60 (m, 4H), 2.89 (dp, J = 13.4, 7.3 Hz, 1H), 2.16 (s, 3H), 2.13-1.99 (m, 4H), 1.80-1.66 (m, 1H). |
| | N-(3-hydroxy-2,6-dimethyl- | | |
| | phenyl)-2-[[1-(tetrahydrofuran- | | |
| | 3-ylmethyl)pyrazol-3- | | |
| | yl]amino]thiazole-5- | | |
| | carboxamide | | |
| BAA- 033 | 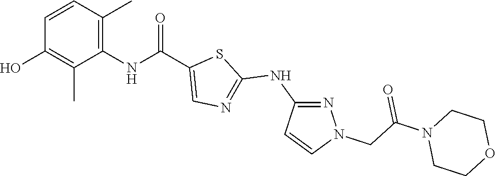 [SMILES: Cc1ccc(O)c(C)c1NC(=O)c1cnc(Nc2ccn(CC(=O)N3CCOCC3)n2)s1] | 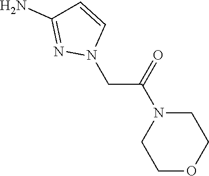 [SMILES: Nc1ccn(CC(=O)N2CCOCC2)n1] | MS (ES+) m/z 457.0 (M + H). ¹H NMR (300 MHz, Methanol-d₄) δ 8.03 (s, 1H), 7.56 (d, J = 2.4 Hz, 1H), 6.91 (d, J = 8.3 Hz, 1H), 6.68 (d, J = 8.2 Hz, 1H), 6.08 (d, J = 2.4 Hz, 1H), 5.08 (s, 2H), 3.69 (dq, J = 10.7, 6.7, 5.0 Hz, 6H), 3.63-3.57 (m, 2H), 2.16 (s, 3H), 2.11 (s, 3H). |
| | N-(3-hydroxy-2,6-dimethyl- | | |
| | phenyl)-2-[1-(2-morpholino-2- | | |
| | oxo-ethyl)pyrazol-3- | | |
| | yl]amino]thiazole-5- | | |
| | carboxamide | | |
| BAA- 034 | 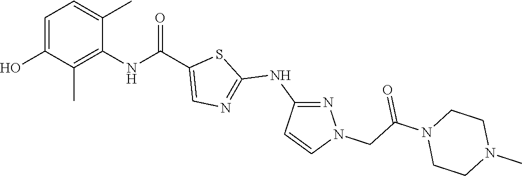 [SMILES: Cc1ccc(O)c(C)c1NC(=O)c1cnc(Nc2ccn(CC(=O)N3CCN(C)CC3)n2)s1] | 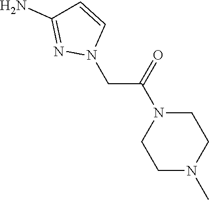 | MS (ES+) m/z 470.4 (M + H). ¹H NMR (300 MHz, Methanol-d₄) δ 8.03 (s, 1H), 7.55 (d, J = 2.4 Hz, 1H), 6.91 (d, J = 8.3 Hz, 1H), 6.68 (d, J = 8.2 Hz, 1H), 6.08 (d, J = 2.4 Hz, 1H), 5.07 (s, 2H), 3.65 (dt, J = 17.1, 5.0 Hz, 4H), 2.47 (dt, J = 18.6, 5.1 Hz, 4H), 2.30 (s, 3H), 2.16 (s, 3H), 2.11 (s, 3H). |
| | N-(3-hydroxy-2,6-dimethyl- | | |
| | phenyl)-2-[1-[2-(4- | | |
| | methylpiperazin-1-yl)-2-oxo- | | |
| | ethyl]pyrazol-3- | | |
| | yl]amino]thiazole-5- | | |
| | carboxamide | | |
| BAA- 035 | 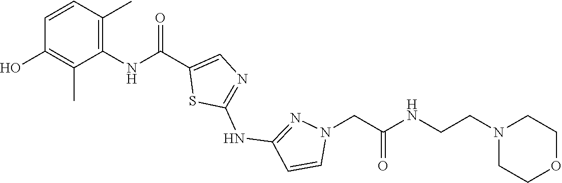 [SMILES: Cc1ccc(O)c(C)c1NC(=O)c1cnc(Nc2ccn(CC(=O)NCCN3CCOCC3)n2)s1] | 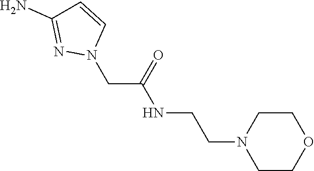 [SMILES: Nc1ccn(CC(=O)NCCN2CCOCC2)n1] | MS (ES+) m/z 500.4 (M + H). ¹H NMR (300 MHz, Methanol-d₄) δ 8.06 (s, 1H), 7.63 (d, J = 2.4 Hz, 1H), 6.91 (d, J = 8.2 Hz, 1H), 6.68 (d, J = 8.2 Hz, 1H), 6.09 (d, J = 2.4 Hz, 1H), 4.80 (s, 2H), 3.59-3.48 (m, 4H), 3.35 (t, J = 6.3 Hz, 2H), 2.46 (t, J = 6.3 Hz, 2H), 2.42-2.31 (m, 4H), 2.16 (s, 3H), 2.11 (s, 3H). |
| | N-(3-hydroxy-2,6-dimethyl- | | |
| | phenyl)-2-[1-[2-(2- | | |
| | morpholinoethylamino)-2-oxo- | | |
| | ethyl]pyrazol-3- | | |
| | yl]amino]thiazole-5- | | |
| | carboxamide | | |
| BAA- 036 | 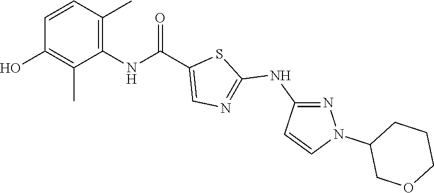 [SMILES: Cc1ccc(O)c(C)c1NC(=O)c1cnc(Nc2ccn(C3CCCOC3)n2)s1] | 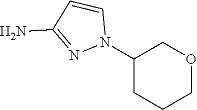 [SMILES: Nc1ccn(C2CCCOC2)n1] | MS (ES+) m/z 414.0 (M). ¹H NMR (300 MHz, Methanol-d₄) δ 7.94 (s, 1H), 7.52 (d, J = 2.0 Hz, 1H), 6.91 (d, J = 8.2 Hz, 1H), 6.68 (d, J = 8.2 Hz, 1H), 6.40-6.22 (m, 1H), 4.56-4.36 (m, 1H), 3.94 (dd, J = 10.6, 4.1 Hz, 2H), 3.68 (t, J = 10.7 Hz, 1H), 3.51-3.39 (m, 1H), 2.12 (d, J = 15.1 Hz, 8H), 1.82 (dq, J = 11.1, 7.5, 5.4 Hz, 2H). |
| | N-(3-hydroxy-2,6-dimethyl- | | |
| | phenyl)-2-[(1-tetrahydropyran- | | |
| | 3-ylpyrazol-3-yl)amino]thiazole- | | |
| | 5-carboxamide | | |
| BAA- 037 | 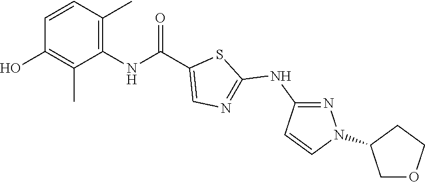 [SMILES: Cc1ccc(O)c(C)c1NC(=O)c1cnc(Nc2ccn([C@@H]3CCOC3)n2)s1] | 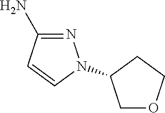 [SMILES: Nc1ccn([C@@H]2CCOC2)n1] | MS (ES+) m/z 400.3 (M + H). ¹H NMR (300 MHz, Methanol-d₄) δ 7.94 (s, 1H), 7.52 (d, J = 2.0 Hz, 1H), 6.91 (d, J = 8.2 Hz, 1H), 6.68 (d, J = 8.2 Hz, 1H), 6.30 (d, J = 2.1 Hz, 1H), 5.15 (tt, J = 8.7, 4.7 Hz, 1H), 4.20-4.03 (m, 2H), 3.98- 3.86 (m, 2H), 2.44-2.27 (m, 2H), 2.14 (s, 3H), 2.09 (s, 3H). |
| | N-(3-hydroxy-2,6-dimethyl- | | |
| | phenyl)-2-[1-[(3R)- | | |
| | tetrahydrofuran-3-yl]pyrazol-3- | | |
| | yl]amino]thiazole-5- | | |
| | carboxamide | | |
| BAA- 038 | 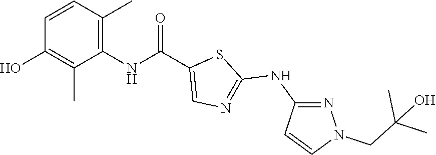 [SMILES: Cc1ccc(O)c(C)c1NC(=O)c1cnc(Nc2ccn(CC(C)(C)O)n2)s1] | 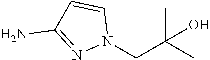 | MS (ES+) m/z 402.0 (M + H). ¹H NMR (300 MHz, Methanol-d₄) δ 8.05 (s, 1H), 7.54 (d, J = 2.4 Hz, 1H), 6.91 (d, J = 8.2 Hz, 1H), 6.68 (d, J = 8.2 Hz, 1H), 6.04 (d, J = 2.4 Hz, 1H), 4.05 (s, 2H), 2.16 (s, 3H), 2.11 (s, 3H), 1.21 (s, 6H). |
| | N-(3-hydroxy-2,6-dimethyl- | | |
| | phenyl)-2-[1-(2-hydroxy-2- | | |
| | methyl-propyl)pyrazol-3- | | |
| | yl]amino]thiazole-5- | | |
| | carboxamide | | |
| BAA- 039 | 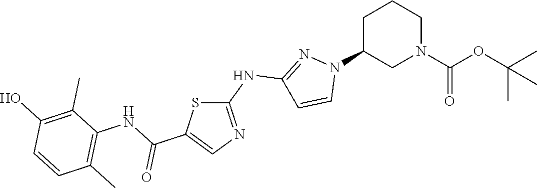 [SMILES: Cc1ccc(O)c(C)c1NC(=O)c1cnc(Nc2ccn([C@H]3CCCN(C(=O)OC(C)(C)C)C3)n2)s1] | 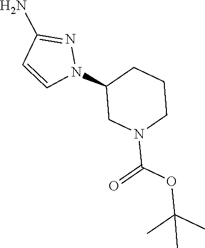 | MS (ES+) m/z 513.4 (M + H). ¹H NMR (300 MHz, Methanol-d₄) 7.96 (s, 1H), 7.56 (d, J = 2.1 Hz, 1H), 6.93 (dt, J = 8.2, 0.7 Hz, 1H), 6.70 (d, J = 8.2 Hz, 1H), 6.34 (d, J = 2.1 Hz, 1H), 4.35 (tt, J = 10.4, 4.7 Hz, 1H), 4.10 (d, J = 13.6 Hz, 2H), 3.21 (s, 1H), 2.91-2.69 (m, 1H), 2.16 (s, 3H), 2.11 (s, 5H), 1.89 (d, J = 13.7 Hz, 1H), 1.71-1.55 (m, 1H), 1.44 (s, 9H). |
| | tert-butyl (3S)-3-[3-[5-[(3- | | |
| | hydroxy-2,6-dimethyl- | | |
| | phenyl)carbamoyl]thiazol-2- | | |
| | yl]amino]pyrazol-1- | | |
| | yl]piperidine-1-carboxylate | | |
| BAA- 040 | 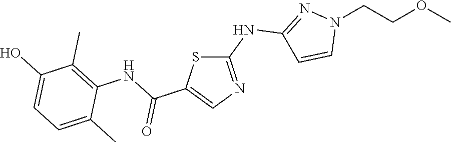 [SMILES: COCCn1ccc(Nc2ncc(C(=O)Nc3c(C)ccc(O)c3C)s2)n1] | 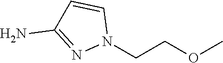 | MS (ES+) m/z 388.1 (M + H). ¹H NMR (300 MHz, DMSO-d₆) δ 11.04 (s, 1H), 9.49 (s, 1H), 9.18 (s, 1H), 8.09 (s, 1H), 7.63 (d, J = 2.3 Hz, 1H), 6.88 (d, J = 8.2 Hz, 1H), 6.68 (d, J = 8.2 Hz, 1H), 5.93 (d, J = 2.3 Hz, 1H), 4.19 (t, J = 5.2 Hz, 2H), 3.70 (t, J = 5.2 Hz, 2H), 3.22 (s, 3H), 2.06 (s, 3H), 1.98 (s, 3H). |
| | N-(3-hydroxy-2,6-dimethyl- | | |
| | phenyl)-2-[1-(2- | | |
| | methoxyethyl)pyrazol-3- | | |
| | yl]amino]thiazole-5- | | |
| | carboxamide | | |
| BAA- 041 | 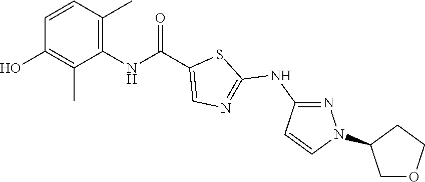 [SMILES: Cc1ccc(O)c(C)c1NC(=O)c1cnc(Nc2ccn([C@H]3CCOC3)n2)s1] | 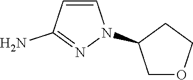 [SMILES: Nc1ccn([C@H]2CCOC2)n1] | MS (ES+) m/z 400.4 (M + H). ¹H NMR (300 MHz, Methanol-d₄) δ 7.96 (s, 1H), 7.54 (s, 1H), 6.92 (d, J = 8.2 Hz, 1H), 6.70 (d, J = 8.2 Hz, 1H), 6.32 (s, 1H), 5.17 (ddt, J = 8.7, 7.1, 4.7 Hz, 1H), 4.21-4.03 (m, 2H), 4.00-3.88 (m, 2H), 2.48- 2.27 (m, 2H), 2.16 (s, 3H), 2.11 (s, 3H). |
| | N-(3-hydroxy-2,6-dimethyl- | | |
| | phenyl)-2-[1-[(3S)- | | |
| | tetrahydrofuran-3-yl]pyrazol-3- | | |
| | yl]amino]thiazole-5- | | |
| | carboxamide | | |
| BAA- 042 | 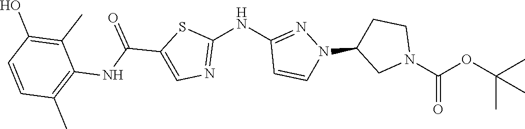 [SMILES: Cc1ccc(O)c(C)c1NC(=O)c1cnc(Nc2ccn([C@H]3CCN(C(=O)OC(C)(C)C)C3)n2)s1] | 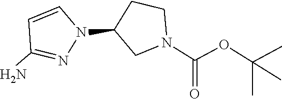 | MS (ES+) m/z 499.4 (M + H). ¹H NMR (300 MHz, Methanol-d₄) δ 7.94 (s, 1H), 7.52 (d, J = 2.0 Hz, 1H), 6.91 (d, J = 8.2 Hz, 1H), 6.68 (d, J = 8.2 Hz, 1H), 6.31 (s, 1H), 5.10 (p, J = 6.6 Hz, 1H), 3.81-3.54 (m, 3H), 3.47 (hept, J = 7.3 Hz, 1H), 2.34 (td, J = 11.2, 10.2, 5.0 Hz, 2H), 2.14 (s, 3H), 2.09 (s, 3H), 1.47 (d, |
| | | | J = 6.1 Hz, 9H). |
| | tert-butyl (3S)-3-[3-[5-[(3- | | |
| | hydroxy-2,6-dimethyl- | | |
| | phenyl)carbamoyl]thiazol-2- | | |
| | yl]amino]pyrazol-1- | | |
| | yl]pyrrolidine-1-carboxylate | | |

Additional example compounds were prepared similarly using Method C with the appropriate amine.

| # | Structure & Name | Analytical Data |
|---|---|---|
| BAA-243 | 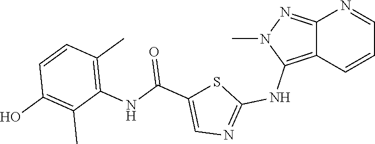 [SMILES: Cc1ccc(O)c(C)c1NC(=O)c1cnc(Nc2c3cccnc3nn2C)s1] | ¹H NMR (300 MHz, DMSO-d₆) δ 12.06 (s, 1H), 9.59 (s, 1H), 9.19 (s, 1H), 8.58 (dd, J = 4.5, 1.6 Hz, 1H), 8.53 (d, J = 8.2 Hz, 1H), 8.23 (s, 1H), 7.20 (dd, J = 8.1, 4.5 Hz, 1H), 6.90 (d, J = 8.2 Hz, 1H), 6.70 (d, J = 8.2 Hz, 1H), 4.01 (s, 3H), 2.10 (s, 3H), 2.02 (s, 3H). MS (ES+) m/z 395.3 (M + H)⁺ |
| | N-(3-hydroxy-2,6-dimethylphenyl)-2- | |
| | ((2-methyl-2H-pyrazolo[3,4-b]pyridin- | |
| | 3-yl)amino)thiazole-5-carboxamide | |
| BAA-244 | 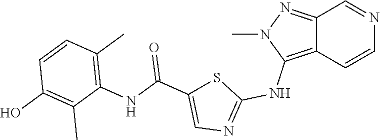 [SMILES: Cc1ccc(O)c(C)c1NC(=O)c1cnc(Nc2c3ccncc3nn2C)s1] | ¹H NMR (300 MHz, Methanol- d4/chloroform-d) δ 8.96 (s, 1H), 8.20 (d, J = 5.7 Hz, 1H), 8.14 (s, 1H), 7.93 (d, J = 5.7 Hz, 1H), 6.92 (d, J = 8.2 Hz, 1H), 6.69 (d, J = 8.2 Hz, 1H), 4.14 (s, 3H), 2.18 (s, 3H), 2.13 (s, 3H). MS (ES+) m/z 395.3 (M + H)⁺ |
| | N-(3-hydroxy-2,6-dimethylphenyl)-2- | |
| | ((2-methyl-2H-pyrazolo[3,4-c]pyridin- | |
| | 3-yl)amino)thiazole-5-carboxamide | |
| BAA-248 | 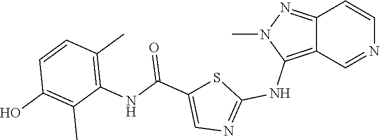 [SMILES: Cc1ccc(O)c(C)c1NC(=O)c1cnc(Nc2c3cnccc3nn2C)s1] | ¹H NMR (300 MHz, DMSO-d₆) δ 12.19 (s, 1H), 9.60 (s, 1H), 9.33 (s, 1H), 9.18 (s, 1H), 8.37 (d, J = 6.1 Hz, 1H), 8.23 (s, 1H), 7.56 (dd, J= 6.1, 1.2 Hz, 1H), 6.90 (d, J = 8.2 Hz, 1H), 6.69 (d, J = 8.2 Hz, 1H), 3.99 (s, 3H), 2.09 (s, 3H), 2.01 (s, 3H). MS (ES+) m/z 395.3 (M + H)⁺ |
| | N-(3-hydroxy-2,6-dimethylphenyl)-2- | |
| | ((2-methyl-2H-pyrazolo[4,3-c]pyridin- | |
| | 3-yl)amino)thiazole-5-carboxamide | |
| BAA-267 | 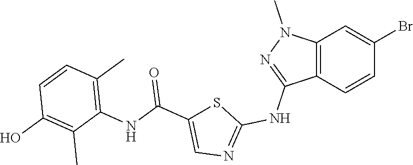 [SMILES: Cc1ccc(O)c(C)c1NC(=O)c1cnc(Nc2nn(C)c3cc(Br)ccc23)s1] | ¹H NMR (300 MHz, Methanol- d4/chloroform-d) δ 8.08 (s, 1H), 7.77 (dd, J = 8.7, 0.6 Hz, 1H), 7.56 (m, 1H), 7.19 (dd, J = 8.6, 1.5 Hz, 1H), 6.89 (d, J = 8.2 Hz, 1H), 6.68 (d, J = 8.2 Hz, 1H), 3.94 (s, 3H), 2.16 (s, 3H), 2.13 (s, 3H). MS (ES+) m/z 472.2/474.2 (M + H)⁺ |
| | 2-((6-bromo-1-methyl-1H-indazol-3- | |
| | yl)amino)-N-(3-hydroxy-2,6- | |
| | dimethylphenyl)thiazole-5- | |
| | carboxamide | |
| BAA-268 | 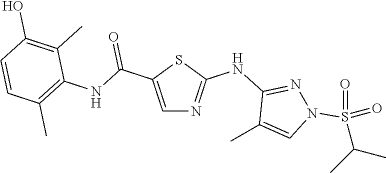 [SMILES: Cc1cn(S(=O)(=O)C(C)C)nc1Nc1ncc(C(=O)Nc2c(C)ccc(O)c2C)s1] | ¹H NMR (300 MHz, Methanol-d4) δ 8.14 (s, 1H), 7.92 (d, J = 1.1 Hz, 1H), 6.94 (d, J = 8.2 Hz, 1H), 6.71 (d, J = 8.2 Hz, 1H), 3.89-3.77 (m, 1H), 2.19 (s, 3H), 2.16 (d, J = 1.1 Hz, 3H), 2.14 (s, 3H), 1.33 (d, J = 6.9 Hz, 6H). MS (ES-) m/z 448.3 [M − H]⁻ |
| | N-(3-hydroxy-2,6-dimethylphenyl)-2- | |
| | ((1-(isopropylsulfonyl)-4-methyl-1H- | |
| | pyrazol-3-yl) amino) thiazole-5- | |
| | carboxamide | |
| BAA-269 | 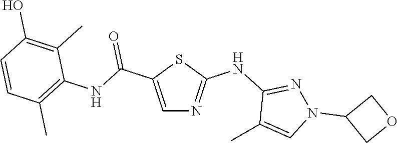 [SMILES: Cc1cn(C2COC2)nc1Nc1ncc(C(=O)Nc2c(C)ccc(O)c2C)s1] | ¹H NMR (300 MHz, MeOD) δ 8.08 (s, 1H), 7.47 (d, J = 0.9 Hz, 1H), 6.94 (d, J = 8.3 Hz, 1H), 6.70 (d, J = 8.2 Hz, 1H), 5.46 (tt, J = 7.5, 6.3 Hz, 1H), 5.17 (t, J = 6.4 Hz, 2H), 5.07-4.95 (m, 2H), 2.19 (s, 3H), 2.14 (s, 3H), 2.09 (d, J = 0.8 Hz, 3H). MS (ES+) m/z 400.3 [M + H]⁺ |
| | N-(3-hydroxy-2,6-dimethylphenyl)-2- | |
| | ((4-methyl-1-(oxetan-3-yl)-1H- | |
| | pyrazol-3-yl)amino) thiazole-5- | |
| | carboxamide | |
| BAA-270 | 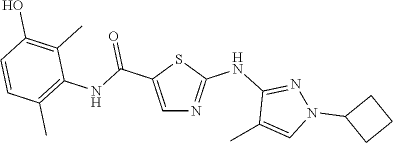 [SMILES: Cc1cn(C2CCC2)nc1Nc1ncc(C(=O)Nc2c(C)ccc(O)c2C)s1] | ¹H NMR (300 MHz, MeOD) δ 8.06 (s, 1H), 7.40 (d, J = 0.9 Hz, 1H), 6.93 (d, J = 8.0 Hz, 1H), 6.70 (d, J = 8.2 Hz, 1H), 4.76-4.63 (m, 1H), 2.70-2.54 (m, 2H), 2.47-2.34 (m, 2H), 2.18 (s, 3H), 2.14 (s, 3H), 2.06 (d, J = 0.8 Hz, 3H), 1.97-1.74 (m, 2H). MS (ES+) m/z 398.1 [M + H]⁺ |
| | 2-((1-cyclobutyl-4-methyl-1H- | |
| | pyrazol-3-yl)amino)-N-(3-hydroxy- | |
| | 2,6-dimethylphenyl)thiazole-5- | |
| | carboxamide | |
| BAA-271 | 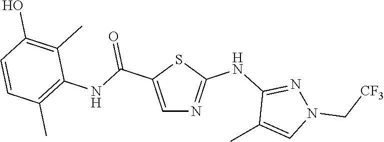 [SMILES: Cc1cn(CC(F)(F)F)nc1Nc1ncc(C(=O)Nc2c(C)ccc(O)c2C)s1] | ¹H NMR (300 MHz, MeOD) δ 8.07 (s, 1H), 7.49 (s, 1H), 6.93 (d, J = 8.2 Hz, 1H), 6.70 (d, J = 8.2 Hz, 1H), 4.82 (q, J = 8.7 Hz, 2H), 2.18 (s, 3H), 2.13 (s, 3H), 2.11 (d, J = 0.9 Hz, 3H). ¹⁹F NMR (282 MHz, MeOD) δ −73.38. MS (ES+) m/z 426.1 [M + H]⁺ |
| | N-(3-hydroxy-2,6-dimethylphenyl)-2- | |
| | ((4-methyl-1-(2,2,2-trifluoroethyl)-1H- | |
| | pyrazol-3-yl) amino) thiazole-5- | |
| | carboxamide | |
| BAA-272 | 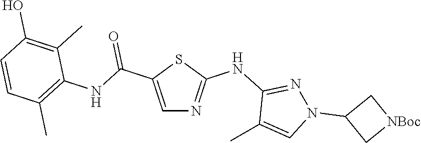 [SMILES: Cc1cn(C2CN(C(=O)OC(C)(C)C)C2)nc1Nc1ncc(C(=O)Nc2c(C)ccc(O)c2C)s1] | ¹H NMR (300 MHz, MeOD) δ 8.06 (s, 1H), 7.47 (d, J = 0.9 Hz, 1H), 6.93 (d, J = 8.3 Hz, 1H), 6.70 (d, J = 8.2 Hz, 1H), 5.08 (p, J = 6.6 Hz, 1H), 4.36 (d, J = 7.4 Hz, 4H), 2.18 (s, 3H), 2.13 (s, 3H), 2.08 (d, J = 0.8 Hz, 3H), 1.48 (s, 9H). MS (ES-) m/z 497.1 [M − H]⁻ |
| | tert-butyl 3-(3-((5-((3-hydroxy-2,6- | |
| | dimethylphenyl) carbamoyl)thiazol-2- | |
| | yl)amino)-4-methyl-1H-pyrazol-1- | |
| | yl)azetidine-1-carboxylate | |

General Method D: Palladium Catalysed Coupling of 2-Bromo-N-(3-Hydroxy-2,6-Dimethyl-Phenyl)Thiazole-5-Carboxamide with Substituted Amines in Aqueous Solvent Mixtures

To a mixture of 2-bromo-N-(3-hydroxy-2,6-dimethyl-phenyl)thiazole-5-carboxamide (1 eq), 5 substituted amino pyrazole (1-3 eq) and Cs₂CO₃ (1.7-3 eq) in 1,4-dioxane: H2O (4:1 or 9:1, 0.06 -0.08 M) was flushed with nitrogen before the addition of Pd₂dba₃ (0.1-0.15 eq) and XantPhos
(0.2-0.25 eq). The mixture was heated to 90-100° C. for 4-18 h. The reaction was monitored by the consumption of starting material via UPLC. The reaction mixture was then cooled and either, filtered through a pad of dicalite, washed with EtOAc/MeOH and the filtrate concentrated in vacuo then partitioned between EtOAc: water or, directly partitioned between EtOAc and water. The 10 organic phase was washed with brine, dried (MgSO₄), filtered and concentrated in vacuo. The crude material was purified by either normal phase chromatography (SiO₂) using a gradient of EtOAc: PE and/or MeOH: DCM and/or MeOH: EtOAc, reverse phase chromatography (C18) using a gradient of MeCN: H₂O and/or by preparative HPLC-MS using a gradient of high or low pH aq. MeCN.

### tert-Butyl(3R)-3-[3-[[5-[(3-hydroxy-2.6-dimethyl-phenyl) carbamoyl]thiazol-2-yl]amino]pyrazol-1-yl]pyrrolidine-1-carboxylate (BAA-043)

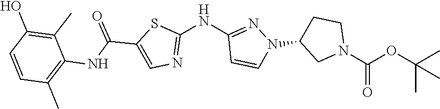 [SMILES: Cc1ccc(O)c(C)c1NC(=O)c1cnc(Nc2ccn([C@@H]3CCN(C(=O)OC(C)(C)C)C3)n2)s1]

Prepared as described in Method D from 2-bromo-N-(3-hydroxy-2,6-dimethyl-phenyl)thiazole-5-carboxamide (130 mg, 0.396 mmol, 1.0 eq), tert-butyl(3R)-3-(3-aminopyrazol-1-yl) pyrrolidine-1-carboxylate (100 mg, 0.396 mmol, 1.0 eq), Pd₂dba₃ (36 mg, 0.040 mmol, 0.1 eq), XantPhos (46 mg, 0.079 mmol, 0.2 eq) and Cs₂CO₃ (220 mg, 0.674 mmol, 1.7 eq) in 1,4-dioxane: H₂O (4:1, 5 mL) to return the title compound (49 mg, 0.098 mmol, 25%) as a pale yellow solid after normal phase chromatography (SiO₂) eluting with 0-20% MeOH: DCM then again with normal phase chromatography (SiO₂) eluting with 60-100% EtOAc: PE then 0-10% MeOH: EtOAc. MS (ES+) m/z 499.3 (M+H). ¹H NMR (300 MHz, Methanol-d4) δ 7.96 (s, 1H), 7.54 (d, J=2.1 Hz, 1H), 6.93 (d, J=8.2 Hz, 1H), 6.70 (d, J=8.2 Hz, 1H), 6.33 (s, 1H), 5.16-5.06 (m, 1H), 3.84-3.56 (m, 3H), 3.56-3.39 (m, 1H), 2.44-2.29 (m, 2H), 2.16 (s, 3H), 2.11 (s, 3H), 1.49 (d, J=6.0 Hz, 9H).

The following example compounds were prepared similarly using Method D using the appropriately substituted amine.

| # | Structure & Name | Amine | Analytical Data |
|---|---|---|---|
| BAA- 044 | 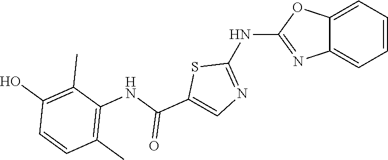 [SMILES: Cc1ccc(O)c(C)c1NC(=O)c1cnc(Nc2nc3ccccc3o2)s1] | 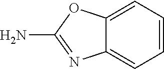 | MS (ES+) m/z 380.8 (M + H). ¹H NMR (300 MHz, Methanol- d₄) δ 8.11 (s, 1H), 7.54-7.47 (m, 1H), 7.47-7.40 (m, 1H), 7.31-7.17 (m, 2H), 6.93 (d, J = 8.3 Hz, 1H), 6.70 (d, J = 8.2 Hz, 1H), 2.17 (s, 3H), 2.12 (s, 3H). |
| | 2-(1,3-benzoxazol-2-ylamino)-N-(3- | | |
| | hydroxy-2,6-dimethyl-phenyl)thiazole-5- | | |
| | carboxamide | | |
| BAA- 045 | 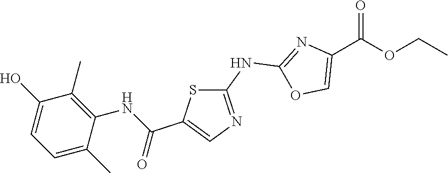 [SMILES: CCOC(=O)c1coc(Nc2ncc(C(=O)Nc3c(C)ccc(O)c3C)s2)n1] | 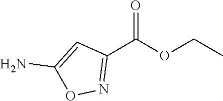 | MS (ES+) m/z 402.9 (M + H). ¹H NMR (300 MHz, Chloroform- d:Methanol-d₄) δ 8.01 (s, 1H), 7.92 (s, 1H), 6.89 (d, J = 8.2 Hz, 1H), 6.68 (d, J = 8.2 Hz, 1H), 4.35 (q, J = 7.1 Hz, 2H), 2.16 (s, 3H), 2.10 (s, 3H), 1.36 (t, J = 7.1 Hz, 3H). |
| | ethyl 2-[[5-[(3-hydroxy-2,6-dimethyl- | | |
| | phenyl)carbamoyl]thiazol-2- | | |
| | yl]aminoJoxazole-4-carboxylate | | |
| BAA- 046 | 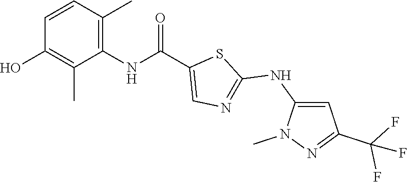 [SMILES: Cc1ccc(O)c(C)c1NC(=O)c1cnc(Nc2cc(C(F)(F)F)nn2C)s1] | 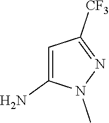 | MS (ES+) m/z 412.3 (M + H). ¹H NMR (300 MHz, DMSO-d₆) δ 11.21-10.81 (br s, 1H), 9.61 (s, 1H), 9.19 (s, 1H), 8.08 (s, 1H), 6.89 (d, J = 8.2 Hz, 1H), 6.86-6.47 (m, 2H), 3.80 (s, 3H), 2.07 (s, 3H), 1.98 (s, 3H). ¹⁹F NMR (282 MHz, DMSO) δ −60.94. |
| | N-(3-hydroxy-2,6-dimethyl-phenyl)-2- | | |
| | [[2-methyl-5-(trifluoromethyl)pyrazol-3- | | |
| | yl]amino]thiazole-5-carboxamide | | |
| BAA- 047 | 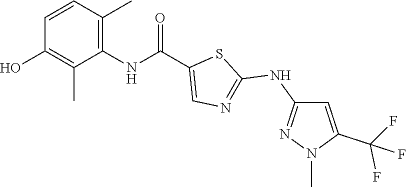 [SMILES: Cc1ccc(O)c(C)c1NC(=O)c1cnc(Nc2cc(C(F)(F)F)n(C)n2)s1] | 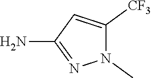 | MS (ES+) m/z 412.3 (M + H). ¹H NMR (300 MHz, DMSO-d₆) δ 11.27 (s, 1H), 9.57 (s, 1H), 9.17 (s, 1H), 8.12 (s, 1H), 6.89 (d, J = 8.2 Hz, 1H), 6.69 (d, J = 8.2 Hz, 1H), 6.60 (s, 1H), 3.92 (s, 3H), 2.07 (s, 3H), 1.99 (s, 3H). ¹⁹F NMR (282 MHz, DMSO-d₆) δ −59.39. |
| | N-(3-hydroxy-2,6-dimethyl-phenyl)-2- | | |
| | [1-methyl-5-(trifluoro methyl) pyrazol-3- | | |
| | yl]amino] thiazole-5-carboxamide | | |
| BAA- 048 | 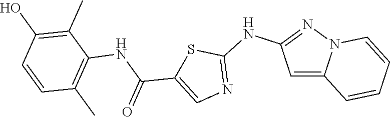 [SMILES: Cc1ccc(O)c(C)c1NC(=O)c1cnc(Nc2cc3ccccn3n2)s1] | 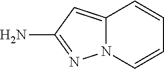 | MS (ES+) m/z 380 (M + H+). ¹H NMR (300 MHz, Methanol- d₄) δ 8.45 (dq, J = 7.0, 1.0 Hz, 1H), 8.09 (s, 1H), 7.48 (dt, J = 8.9, 1.2 Hz, 1H), 7.19 (ddd, J = 8.9, 6.8, 1.1 Hz, 1H), 6.96-6.90 (m, 1H), 6.76 (td, J = |
| | | | 6.9, 1.4 Hz, 1H), 6.69 |
| | N-(3-hydroxy-2,6-dimethyl-phenyl)-2- | | (d, J = 8.2 Hz, 1H), 6.29 |
| | (pyrazolo[1,5-a]pyridin-2- | | (d, J = 0.8 Hz, 1H), 2.21- |
| | ylamino)thiazole-5-carboxamide | | 2.16 (m, 3H), 2.13 |
| | | | (s, 3H). |
| BAA- 049 | 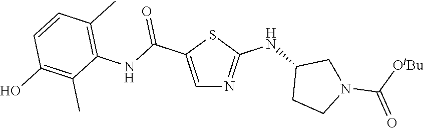 [SMILES: Cc1ccc(O)c(C)c1NC(=O)c1cnc(N[C@H]2CCN(C(=O)OC(C)(C)C)C2)s1] | 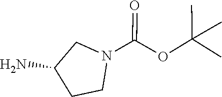 | MS (ES+) m/z 433.3 (M + H). ¹H NMR (300 MHz, Methanol- d₄) δ 7.89 (s, 1H), 6.90 (d, 1H, J = 8.2 Hz), 6.67 (d, 1H, J = 8.2 Hz), 4.34 (p, 1H, J = 5.5 Hz), 3.65 (dt, 1H, J = 10.8, 5.2 Hz), 3.50-3.44 (m, |
| | | | 2H), 3.39-3.35 (m, 1H), |
| | tert-butyl (3S)-3-[5-[(3-hydroxy-2,6- | | 2.30-2.19 (m, 1H), 2.14 |
| | dimethyl-phenyl)carbamoyl]thiazol-2- | | (s, 3H), 2.09 (s, 3H), |
| | ylamino]pyrrolidine-1-carboxylate | | 2.05-1.98 (m, 1H), |
| | | | 1.47 (s, 9H) ppm. |
| BAA- 050 | 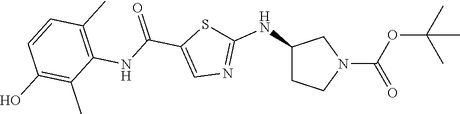 [SMILES: Cc1ccc(O)c(C)c1NC(=O)c1cnc(N[C@@H]2CCN(C(=O)OC(C)(C)C)C2)s1] | 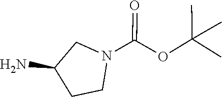 | MS (ES+) m/z 433.3 {M + H). ¹H NMR (300 MHz, Methanol-d₄) δ 7.89 (s, 1H), 6.90 (d, 1H, J = 8.2 Hz), 6.67 (d, 1H, J = 8.2 Hz), 4.34 (p, 1H, J = 5.6 Hz), 3.65 (dt, 1H, J = 10.7, |
| | | | 5.3 Hz), 3.49-3.44 (m, |
| | tert-butyl (3R)-3-[5-[(3-hydroxy-2,6- | | 2H), 3.39-3.35 (m, 1H), |
| | dimethyl-phenyl)carbamoyl]thiazol-2- | | 2.30-2.19 (m, 1H), 2.14 |
| | yl]amino]pyrrolidine-1-carboxylate | | (s, 3H), 2.09 (s, 3H), , |
| | | | 2.05-1.97 (m, 1H) |
| | | | 1.47 (s, 9H) ppm. |
| BAA- 051 | 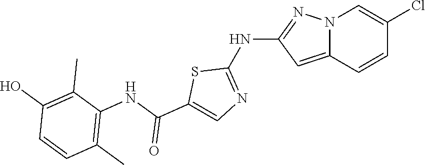 [SMILES: Cc1ccc(O)c(C)c1NC(=O)c1cnc(Nc2cc3ccc(Cl)cn3n2)s1] | 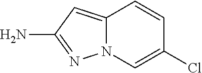 [SMILES: Nc1cc2ccc(Cl)cn2n1] | MS (ES+) m/z 414/416 (M + H), Cl isotope pattern. ¹H NMR (300 MHz, Methanol-d₄) δ 8.56 (s, 1H), 8.08 (t, J = 1.9 Hz, 1H), 7.77 (s, 1H), 7.45 (d, J = 9.6 Hz, 1H), 7.17 (d, J = 9.4 Hz, 1H), 6.91 (d, J = 8.1 Hz, 1H), 6.69 |
| | | | (dd, J = 7.9, 1.9 Hz, |
| | 2-[(6-chloropyrazolo[1,5-a]pyridin-2- | | 1H), 6.36 (s, 1H), 2.18 |
| | yl)amino]-N-(3-hydroxy-2,6-dimethyl- | | (d, J = 2.0 Hz, 3H), 2.13 |
| | phenyl)thiazole-5-carboxamide | | (d, J = 1.9 Hz, 3H). |

Additional example compounds were prepared similarly using Method D with an appropriate amine.

| # | Structure & Name | Analytical Data |
|---|---|---|
| BAA-359 | 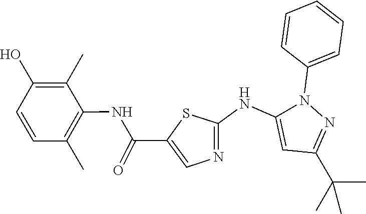 [SMILES: Cc1ccc(O)c(C)c1NC(=O)c1cnc(Nc2cc(C(C)(C)C)nn2-c2ccccc2)s1] | MS (ES+) m/z 462 (M + H). ¹H NMR (300 MHz, Methanol-d4) δ 7.91 (s, 1H), 7.58-7.45 (m, 4H), 7.45- 7.34 (m, 1H), 6.93-6.88 (m, 1H), 6.68 (d, J = 8.2 Hz, 1H), 6.46 (s, 1H), 2.13 (s, 3H), 2.09 (s, 3H), 1.37 (s, 9H). |
| | 2-((3-(tert-butyl)-1-phenyl-1H-pyrazol-5- | |
| | yl)amino)-N-(3-hydroxy-2,6- | |
| | dimethylphenyl)thiazole-5-carboxamide | |
| BAA-361 | 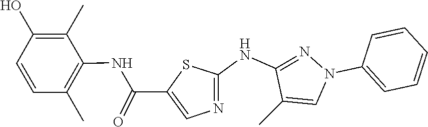 [SMILES: Cc1cn(-c2ccccc2)nc1Nc1ncc(C(=O)Nc2c(C)ccc(O)c2C)s1] | MS (ES+) m/z 420 (M + H). ¹H NMR (300 MHz, Methanol-d4) δ 8.10 (s, 1H), 7.91 (s, 1H), 7.79-7.71 (m, 2H), 7.47-7.39 (m, 2H), 7.24- 7.14 (m, 1H), 6.92 (d, J = 8.2 Hz, 1H), 6.71 (d, J = 8.2 Hz, 1H), 2.20 (s, 3H), 2.18 (d, J = 0.8 Hz, 3H), 2.16 (s, 3H). |
| | N-(3-hydroxy-2,6-dimethylphenyl)-2-((4- | |
| | methyl-1-phenyl-1H-pyrazol-3- | |
| | yl)amino)thiazole-5-carboxamide | |
| BAA-365 | 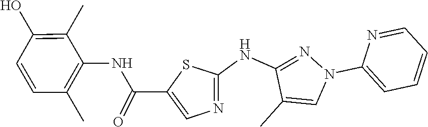 [SMILES: Cc1cn(-c2ccccn2)nc1Nc1ncc(C(=O)Nc2c(C)ccc(O)c2C)s1] | MS (ES+) m/z 421 (M + H). ¹H NMR (300 MHz, Methanol-d4) δ 8.31 (d, J = 4.7 Hz, 1H), 8.27 (s, 1H), 7.97 (d, J = 8.3 Hz, 1H), 7.85 (dd, J = 8.5, 6.7 Hz, 1H), 7.65 (t, J = 4.2 Hz, 1H), 7.15 (dd, J = 7.3, 5.0 Hz, 1H), 6.91 (d, J = 8.2 Hz, 1H), 6.69 (d, J = 8.2 Hz, 1H), 2.18 (s, 6H), 2.14 (s, 3H). |
| | N-(3-hydroxy-2,6-dimethylphenyl)-2-((4- | |
| | methyl-1-(pyridin-2-yl)-1H-pyrazol-3- | |
| | yl)amino)thiazole-5-carboxamide | |
| BAA-366 | 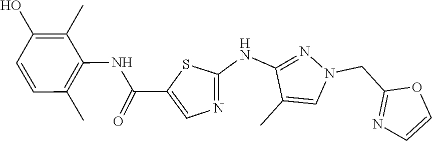 [SMILES: Cc1cn(Cc2ncco2)nc1Nc1ncc(C(=O)Nc2c(C)ccc(O)c2C)s1] | MS (ES+) m/z 425 (M + H). ¹H NMR (300 MHz, MeOD) δ 8.01 (s, 1H), 7.90 (d, J = 0.9 Hz, 1H), 7.49 (d, J = 0.9 Hz, 1H), 7.16 (d, J = 0.9 Hz, 1H), 6.91 (d, J = 8.2 Hz, 1H), 6.68 (d, J = 8.2 Hz, 1H), 5.39 (s, 2H), 2.15 (s, 3H), 2.11 (s, 3H), 2.07 (d, J = 0.9 Hz, 3H). |
| | N-(3-hydroxy-2,6-dimethylphenyl)-2-((4- | |
| | methyl-1-(oxazol-2-ylmethyl)-1H-pyrazol- | |
| | 3-yl)amino) thiazole-5-carboxamide | |
| BAA-367 | 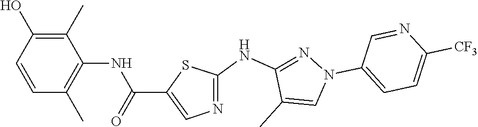 [SMILES: Cc1cn(-c2ccc(C(F)(F)F)nc2)nc1Nc1ncc(C(=O)Nc2c(C)ccc(O)c2C)s1] | MS (ES+) m/z 489 (M + H). ¹H NMR (300 MHz, MeOD) δ 8.99 (d, J = 2.5 Hz, 1H), 8.31-8.21 (m, 1H), 7.92 (s, 1H), 7.79 (dd, J = 16.8, 7.8 Hz, 2H), 6.89 (q, J = 7.0 Hz, 1H), 6.68 (d, J = 8.2 Hz, 1H), 2.18- 2.11 (m, 9H). 19F NMR (282 MHz, MeOD) δ −-67.62. |
| | N-(3-hydroxy-2,6-dimethylphenyl)-2-((4- | |
| | methyl-1-(6-(trifluoromethyl)pyridin-3-yl)- | |
| | 1H-pyrazol-3-yl)amino)thiazole-5- | |
| | carboxamide | |
| BAA-368 | 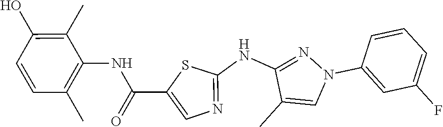 [SMILES: Cc1cn(-c2cccc(F)c2)nc1Nc1ncc(C(=O)Nc2c(C)ccc(O)c2C)s1] | MS (ES+) m/z 438 (M + H). ¹H NMR (300 MHz, MeOD) δ 7.99 (s, 1H), 7.73 (d, J = 1.1 Hz, 1H), 7.50- 7.27 (m, 3H), 6.92-6.75 (m, 2H), 6.67 (d, J = 8.2 Hz, 1H), 2.15 (d, J = 2.7 Hz, 3H), 2.13 (s, 3H), 2.12 (s, 3H). |
| | 2-((1-(3-fluorophenyl)-4-methyl-1H- | |
| | pyrazol-3-yl)amino)-N-(3-hydroxy-2,6- | |
| | dimethylphenyl)thiazole-5-carboxamide | |
| BAA-369 | 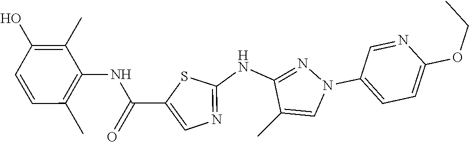 [SMILES: CCOc1ccc(-n2cc(C)c(Nc3ncc(C(=O)Nc4c(C)ccc(O)c4C)s3)n2)cn1] | MS (ES+) m/z 465 (M + H). ¹H NMR (300 MHz, MeOD) δ 8.52 (s, 1H), 8.18-7.99 (m, 2H), 7.95 (s, 1H), 6.92 (d, J = 8.3 Hz, 1H), 6.84 (d, J = 8.9 Hz, 1H), 6.69 (d, J = 8.2 Hz, 1H), 4.32 (q, J = 7.0 Hz, 2H), 2.21-2.15 (m, 6H), 2.13 (s, 3H), 1.37 (t, J = 7.0 Hz, 3H). |
| | 2-((1-(6-ethoxypyridin-3-yl)-4-methyl-1H- | |
| | pyrazol-3-yl)amino)-N-(3-hydroxy-2,6- | |
| | dimethylphenyl)thiazole-5-carboxamide | |
| BAA-370 | 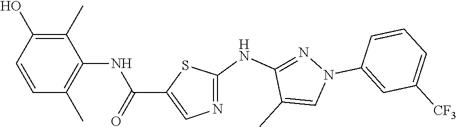 [SMILES: Cc1cn(-c2cccc(C(F)(F)F)c2)nc1Nc1ncc(C(=O)Nc2c(C)ccc(O)c2C)s1] | MS (ES+) m/z 489 (M + H). ¹H NMR (300 MHz, MeOD) δ 8.07 (t, J = 1.2 Hz, 1H), 8.00 (d, J = 8.3 Hz, 1H), 7.91 (d, J = 13.9 Hz, 2H), 7.56 (d, J = 7.9 Hz, 1H), 7.42 (d, J = 7.9 Hz, 1H), 6.90 (d, J = 8.2 Hz, 1H), 6.68 (d, J = 8.2 Hz, 1H), 2.17 (s, 6H), 2.13 (s, 3H). ¹⁹F NMR (282 MHz, MeOD) δ 63.51. |
| | N-(3-hydroxy-2, 6-dimethylphenyl)-2-((4- | |
| | methyl-1-(3-(trifluoromethyl)phenyl)-1H- | |
| | pyrazol-3-yl)amino)thiazole-5- | |
| | carboxamide | |
| BAA-371 | 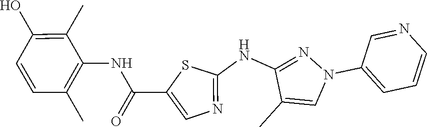 [SMILES: Cc1cn(-c2cccnc2)nc1Nc1ncc(C(=O)Nc2c(C)ccc(O)c2C)s1] | MS (ES+) m/z 421 (M + H). ¹H NMR (300 MHz, MeOD) δ 9.03 (dd, J = 2.7, 0.8 Hz, 1H), 8.37 (dd, J = 4.8, 1.4 Hz, 1H), 8.30-8.23 (m, 1H), 8.16 (d, J = 1.0 Hz, 1H), 8.12 (s, 1H), 7.52 (ddd, J = 8.4, 4.8, 0.8 Hz, 1H), 6.92 (d, J = 8.2 Hz, 1H), 6.70 (d, J = 8.2 Hz, 1H), 2.20 |
| | | (d, J = 0.9 Hz, 3H), 2.19-2.18 (m, |
| | N-(3-hydroxy-2, 6-dimethylphenyl)-2-((4- | 3H), 2.14 (s, 3H). |
| | methyl-1-(pyridin-3-yl)-1H-pyrazol-3- | |
| | yl)amino)thiazole-5-carboxamide | |
| BAA-372 | 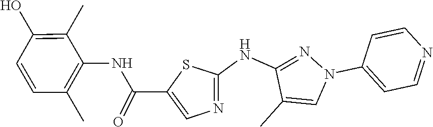 [SMILES: Cc1cn(-c2ccncc2)nc1Nc1ncc(C(=O)Nc2c(C)ccc(O)c2C)s1] | MS (ES+) m/z 421 (M + H). ¹H NMR (300 MHz, MeOD) δ 8.55-8.47 (m, 2H), 8.21 (s, 1H), 8.14 (s, 1H), 7.86-7.77 (m, 2H), 6.93 (d, J = 8.3 Hz, 1H), 6.70 (d, J = 8.2 Hz, 1H), 2.20 (s, 3H), 2.19 (s, 3H), 2.15 (s, 3H). |
| | N-(3-hydroxy-2,6-dimethylphenyl)-2-((4- | |
| | methyl-1-(pyridin-4-yl)-1H-pyrazol-3- | |
| | yl)amino)thiazole-5-carboxamide | |
| BAA-373 | 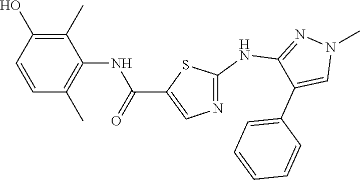 [SMILES: Cc1ccc(O)c(C)c1NC(=O)c1cnc(Nc2nn(C)cc2-c2ccccc2)s1] | MS (ES+) m/z 420 (M + H). ¹H NMR (300 MHz, MeOD) δ 7.97 (s, 1H), 7.78 (s, 1H), 7.55-7.45 (m, 2H), 7.38 (ddd, J = 7.7, 6.9, 1.2 Hz, 2H), 7.32-7.20 (m, 1H), 6.99- 6.86 (m, 1H), 6.68 (d, J = 8.2 Hz, 1H), 3.91 (s, 3H), 2.15-2.14 (m, 3H), 2.10 (s, 3H). |
| | N-(3-hydroxy-2,6-dimethylphenyl)-2-((1- | |
| | methyl-4-phenyl-1H-pyrazol-3- | |
| | yl)amino)thiazole-5-carboxamide | |
| BAA-382 | 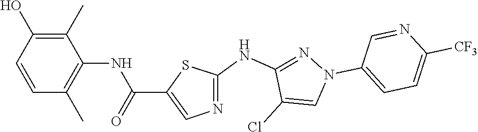 [SMILES: Cc1ccc(O)c(C)c1NC(=O)c1cnc(Nc2nn(-c3ccc(C(F)(F)F)nc3)cc2Cl)s1] | MS (ES+) m/z 509, 511 (M + H). 1H NMR (300 MHz, MeOD) δ 9.19 (t, J = 3.1 Hz, 1H), 8.61 (d, J = 4.7 Hz, 1H), 8.41 (dt, J = 8.6, 2.9 Hz, 1H), 8.13 (s, 1H), 7.88 (d, J = 8.7 Hz, 1H), 6.91 (d, J = 8.3 Hz, 1H), 6.69 (d, J = 8.2 Hz, 1H), 2.18 (s, 3H), 2.14 (s, 3H). 19F NMR (282 MHz, MeOD) δ 68.51. |
| | 2-((4-chloro-1-(6-(trifluoromethyl) pyridin- | |
| | 3-yl)-1H-pyrazol-3-yl)amino)-N-(3- | |
| | hydroxy-2,6-dimethylphenyl) thiazole-5- | |
| | carboxamide | |
| BAA-383 | 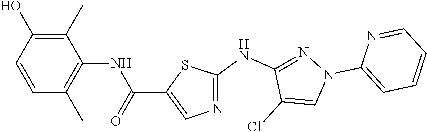 [SMILES: Cc1ccc(O)c(C)c1NC(=O)c1cnc(Nc2nn(-c3ccccn3)cc2Cl)s1] | MS (ES+) m/z 441, 443 (M + H). 1H NMR (300 MHz, MeOD) δ 8.58 (s, 1H), 8.13 (d, J = 2.0 Hz, 1H), 8.06- 7.96 (m, 1H), 7.92 (d, J = 8.0 Hz, 1H), 7.72 (s, 1H), 7.23 (s, 1H), 6.91 (d, J = 8.2 Hz, 1H), 6.72- 6.67 (m, 1H), 2.16 (s, 3H), 2.14 (s, 3H). |
| | 2-((4-chloro-1-(pyridin-2-yl)-1H-pyrazol-3- | |
| | yl)amino)-N-(3-hydroxy-2,6- | |
| | dimethylphenyl) thiazole-5-carboxamide | |
| BAA-384 | 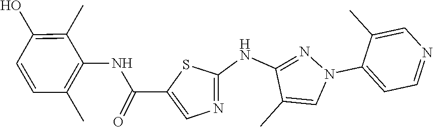 [SMILES: Cc1cnccc1-n1cc(C)c(Nc2ncc(C(=O)Nc3c(C)ccc(O)c3C)s2)n1] | MS (ES+) m/z 435 (M + H). ¹H NMR (300 MHz, MeOD) δ 8.47 (s, 1H), 8.42 (d, J = 5.5 Hz, 1H), 8.12 (s, 1H), 7.98 (d, J = 1.1 Hz, 1H), 7.63 (d, J = 5.5 Hz, 1H), 6.92 (d, J = 8.3 Hz, 1H), 6.69 (d, J = 8.2 Hz, 1H), 2.62 (s, 3H), 2.20 (s, 3H), 2.18- 2.16 (m, 3H), 2.12 (s, 3H). |
| | N-(3-hydroxy-2,6-dimethylphenyl)-2-((4- | |
| | methyl-1-(3-methylpyridin-4-yl)-1H- | |
| | pyrazol-3-yl)amino)thiazole-5- | |
| | carboxamide | |
| BAA-385 | 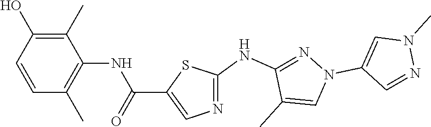 [SMILES: Cc1cn(-c2cnn(C)c2)nc1Nc1ncc(C(=O)Nc2c(C)ccc(O)c2C)s1] | MS (ES+) m/z 424 (M + H). ¹H NMR (300 MHz, MeOD) δ 8.07 (s, 1H), 7.95 (d, J = 0.9 Hz, 1H), 7.77 (d, J = 0.8 Hz, 1H), 7.72 (d, J = 0.9 Hz, 1H), 6.92 (d, J = 8.3 Hz, 1H), 6.69 (d, J = 8.3 Hz, 1H), 3.91 (s, 3H), 2.18 (dd, J = 1.4, 0.8 Hz, 3H), 2.15-2.11 (m, 6H). |
| | 2-((1′′,4-dimethyl-1′H-[1,4′-bipyrazol]-3- | |
| | yl)amino)-N-(3-hydroxy-2,6- | |
| | dimethylphenyl)thiazole-5-carboxamide | |
| BAA-386 | 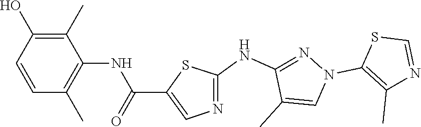 [SMILES: Cc1cn(-c2scnc2C)nc1Nc1ncc(C(=O)Nc2c(C)ccc(O)c2C)s1] | MS (ES+) m/z 441 (M + H). ¹H NMR (300 MHz, MeOD) δ 8.76 (s, 1H), 8.09 (s, 1H), 7.73 (q, J = 0.9 Hz, 1H), 6.91 (dt, J = 8.2, 0.7 Hz, 1H), 6.68 (d, J = 8.2 Hz, 1H), 2.19- 2.13 (m, 9H), 2.11 (s, 3H). |
| | N-(3-hydroxy-2,6-dimethylphenyl)-2-((4- | |
| | methyl-1-(4-methylthiazol-5-yl)-1H- | |
| | pyrazol-3-yl)amino)thiazole-5- | |
| | carboxamide | |
| BAA-387 | 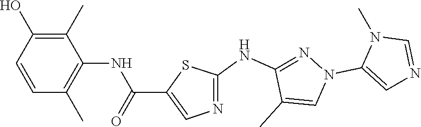 [SMILES: Cc1cn(-c2cncn2C)nc1Nc1ncc(C(=O)Nc2c(C)ccc(O)c2C)s1] | MS (ES+) m/z 424 (M + H). ¹H NMR (300 MHz, MeOD) δ 8.08 (s, 1H), 7.66 (dd, J = 1.2, 0.6 Hz, 1H), 7.62 (d, J = 1.0 Hz, 1H), 7.04 (d, J = 1.2 Hz, 1H), 6.95-6.87 (m, 1H), 6.68 (d, J = 8.2 Hz, 1H), 3.76-3.70 (m, 3H), 2.16 (dd, J = 2.2, 1.1 Hz, 6H), 2.10 (s, 3H). |
| | N-(3-hydroxy-2, 6-dimethylphenyl)-2-((4- | |
| | methyl-1-(1-methyl-1H-imidazol-5-yl)-1H- | |
| | pyrazol-3-yl)amino)thiazole-5- | |
| | carboxamide | |
| BAA-389 | 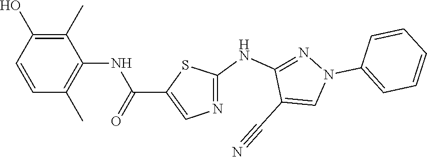 | MS (ES+) m/z 424 (M + H). ¹H NMR (300 MHz, MeOD) δ 8.08 (s, 1H), 7.66 (dd, J = 1.2, 0.6 Hz, 1H), 7.62 (d, J = 1.0 Hz, 1H), 7.04 (d, J = 1.2 Hz, 1H), 6.95-6.87 (m, 1H), 6.68 (d, J = 8.2 Hz, 1H), 3.76-3.70 (m, 3H), 2.16 (dd, J = 2.2, 1.1 Hz, 6H), 2.10 (s, 3H). |
| | 2-((4-cyano-1-phenyl-1H-pyrazol-3- | |
| | yl)amino)-N-(3-hydroxy-2,6- | |
| | dimethylphenyl)thiazole-5-carboxamide | |
| BAA-391 | 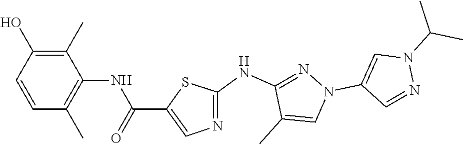 [SMILES: Cc1cn(-c2cnn(C(C)C)c2)nc1Nc1ncc(C(=O)Nc2c(C)ccc(O)c2C)s1] | MS (ES+) m/z 452 (M + H). 1H NMR (300 MHz, MeOD) δ 8.07 (s, 1H), 8.00 (s, 1H), 7.78 (s, 1H), 7.72 (d, J = 1.5 Hz, 1H), 6.92 (d, J = 8.3 Hz, 1H), 6.69 (d, J = 8.2 Hz, 1H), 4.53 (hept, J = 6.7 Hz, 1H), 2.17 (s, 3H), 2.13 (d, J = 2.2 Hz, 6H), 1.53 (d, J = 1.1 Hz, 3H), 1.50 (d, J = 1.1 Hz, 3H). |
| | N-(3-hydroxy-2, 6-dimethylphenyl)-2-((1′- | |
| | isopropyl-4-methyl-1′H-[1,4′-bipyrazol]-3- | |
| | yl)amino)thiazole-5-carboxamide | |
| BAA-392 | 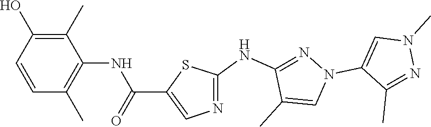 [SMILES: Cc1cn(-c2cn(C)nc2C)nc1Nc1ncc(C(=O)Nc2c(C)ccc(O)c2C)s1] | MS (ES+) m/z 438 (M + H). ¹H NMR (300 MHz, MeOD) δ 8.06 (s, 1H), 7.81 (s, 1H), 7.57 (d, J = 0.9 Hz, 1H), 6.95-6.88 (m, 1H), 6.68 (d, J = 8.2 Hz, 1H), 3.84 (s, 3H), 2.37 (s, 3H), 2.16 (d, J = 1.3 Hz, 3H), 2.14 (d, J = 0.9 Hz, 3H), 2.11 (s, 3H). |
| | N-(3-hydroxy-2,6-dimethylphenyl)-2- | |
| | ((1′, 3′,4-trimethyl-1′H-[1,4′-bipyrazol]-3- | |
| | yl)amino)thiazole-5-carboxamide | |
| BAA-393 | 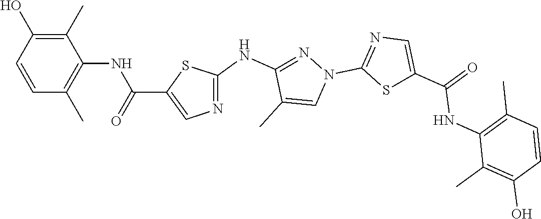 [SMILES: Cc1cn(-c2ncc(C(=O)Nc3c(C)ccc(O)c3C)s2)nc1Nc1ncc(C(=O)Nc2c(C)ccc(O)c2C)s1] | MS (ES+) m/z 590 (M + H). ¹H NMR (300 MHz, DMSO) δ 11.56 (s, 1H), 9.87 (s, 1H), 9.53 (s, 1H), 9.20 (s, 1H), 9.15 (s, 1H), 8.32 (s, 1H), 8.30 (s, 1H), 8.21 (s, 1H), 6.88 (dd, J = 8.2, 3.3 Hz, 2H), 6.69 (dd, J = 8.2, 5.0 Hz, 2H), 2.17- 2.12 (m, 3H), 2.08 (d, J = 1.6 Hz, 6H), 1.99 (s, 6H). |
| | N-(3-hydroxy-2,6-dimethylphenyl)-2-((1- | |
| | (5-((3-hydroxy-2,6-dimethylphenyl) | |
| | carbamoyl)thiazol-2-yl)-4-methyl-1H- | |
| | pyrazol-3-yl)amino)thiazole-5- | |
| | carboxamide | |
| BAA-394 | 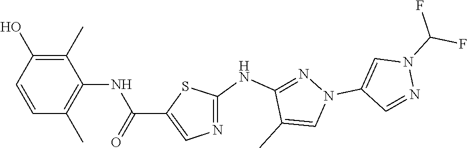 [SMILES: Cc1cn(-c2cnn(C(F)F)c2)nc1Nc1ncc(C(=O)Nc2c(C)ccc(O)c2C)s1] | MS (ES+) m/z 460 (M + H). 1H NMR (300 MHz, MeOD) δ 8.36 (d, J = 0.7 Hz, 1H), 8.13-8.04 (m, 2H), 7.83 (q, J = 0.9 Hz, 1H), 7.48 (t, J = 59.5 Hz, 1H), 6.92 (d, J = 8.3 Hz, 1H), 6.69 (d, J = 8.2 Hz, 1H), 2.18 (s, 3H), 2.15 (d, J = 0.9 Hz, 3H), 2.13 (s, 3H). ¹⁹F NMR (282 MHz, MeOD) δ 96.15. |
| | 2-((1′-(difluoromethyl)-4-methyl-1′H-[1,4′- | |
| | bipyrazol]-3-yl)amino)-N-(3-hydroxy-2,6- | |
| | dimethylphenyl)thiazole-5-carboxamide | |
| BAA-395 | 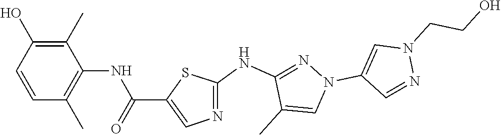 [SMILES: Cc1cn(-c2cnn(CCO)c2)nc1Nc1ncc(C(=O)Nc2c(C)ccc(O)c2C)s1] | MS (ES+) m/z 454 (M + H). ¹H NMR (300 MHz, MeOD) ? 6.52 (s, 1H), 6.46 (d, J = 0.8 Hz, 1H), 6.27 (d, J = 0.8 Hz, 1H), 6.18 (d, J = 0.9 Hz, 1H), 5.37 (d, J = 8.2 Hz, 1H), 5.14 (d, J = 8.2 Hz, 1H), 2.69 (t, J = 5.3 Hz, 2H), 2.36 (dd, J = 5.8, 4.9 Hz, 2H), 0.63 (s, 3H), 0.59 |
| | | (s, 3H), 0.58 (s, 3H). |
| | N-(3-hydroxy-2,6-dimethylphenyl)-2-((1′- | |
| | (2-hydroxyethyl)-4-methyl-1′H-[1,4′- | |
| | bipyrazol]-3-yl)amino)thiazole-5- | |
| | carboxamide | |
| BAA-398 | 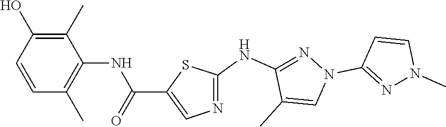 [SMILES: Cc1cn(-c2ccn(C)n2)nc1Nc1ncc(C(=O)Nc2c(C)ccc(O)c2C)s1] | MS (ES+) m/z 424 (M + H). ¹H NMR (300 MHz, MeOD) δ 8.05 (s, 1H), 7.82 (s, 1H), 7.48 (s, 1H), 6.90 (d, J = 8.3 Hz, 1H), 6.68 (d, J = 8.2 Hz, 1H), 6.49 (d, J = 2.3 Hz, 1H), 3.86 (d, J = 1.1 Hz, 3H), 2.16 (d, J = 1.0 Hz, 3H), 2.14 (s, 3H), 2.12 (s, 3H). |
| | 2-((1′,4-dimethyl-1′H-[1,3′-bipyrazol]-3- | |
| | yl)amino)-N-(3-hydroxy-2,6- | |
| | dimethylphenyl)thiazole-5-carboxamide | |
| BAA-231 | 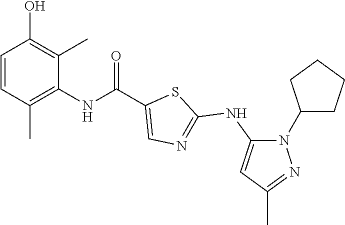 [SMILES: Cc1cc(Nc2ncc(C(=O)Nc3c(C)ccc(O)c3C)s2)n(C2CCCC2)n1] | 1H NMR (300 MHz, Methanol-d4) δ 7.95 (s, 1H), 6.92 (d, J = 8.2 Hz, 1H), 6.70 (d, J = 8.2 Hz, 1H), 6.14 (s, 1H), 4.73 (p, J = 7.7 Hz, 1H), 2.26 (s, 3H), 2.16 (s, 3H), 2.11 (s, 3H), 2.09-1.87 (m, 6H), 1.69 (qq, J = 8.6, 5.2, 4.4 Hz, 2H). MS (ES+) m/z 412.4 (M + H). |
| | 2-((1-cyclopentyl-3-methyl-1H-pyrazol-5- | |
| | yl)amino)-N-(3-hydroxy-2,6- | |
| | dimethylphenyl)thiazole-5-carboxamide | |
| BAA-232 | 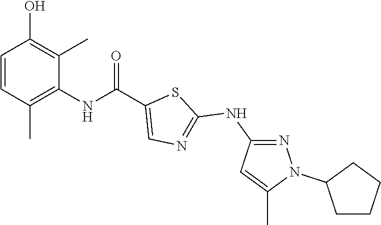 [SMILES: Cc1ccc(O)c(C)c1NC(=O)c1cnc(Nc2cc(C)n(C3CCCC3)n2)s1] | 1H NMR (300 MHz, Methanol-d4) δ 8.03 (s, 1H), 6.91 (d, J = 8.2 Hz, 1H), 6.68 (d, J = 8.2 Hz, 1H), 5.75 (s, 1H), 4.68-4.58 (m, 1H), 2.29 (s, 3H), 2.17 (s, 3H), 2.12 (s, 3H), 2.10-1.95 (m, 6H), 1.71 (d, J = 6.4 Hz, 2H). MS (ES+) m/z 412.4 (M + H). |
| | 2-((1-cyclopentyl-5-methyl-1H-pyrazol-3- | |
| | yl)amino)-N-(3-hydroxy-2,6- | |
| | dimethylphenyl)thiazole-5-carboxamide | |
| BAA-233 | 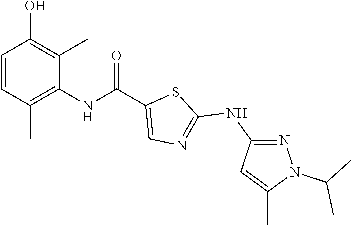 [SMILES: Cc1ccc(O)c(C)c1NC(=O)c1cnc(Nc2cc(C)n(C(C)C)n2)s1] | 1H NMR (300 MHz, Methanol-d4) δ 8.03 (s, 1H), 6.91 (d, J = 8.2 Hz, 1H), 6.68 (d, J = 8.2 Hz, 1H), 5.75 (s, 1H), 4.47 (p, J = 6.6 Hz, 1H), 2.28 (s, 3H), 2.17 (s, 3H), 2.12 (s, 3H), 1.46 (d, J = 6.6 Hz, 6H). MS (ES+) m/z 386.0 (M + H). |
| | N-(3-hydroxy-2, 6-dimethylphenyl)-2-((1- | |
| | isopropyl-5-methyl-1H-pyrazol-3- | |
| | yl)amino)thiazole-5-carboxamide | |
| BAA-234 | 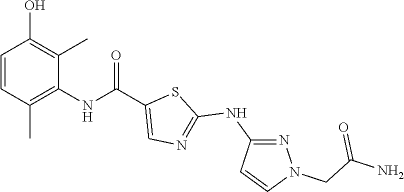 [SMILES: Cc1ccc(O)c(C)c1NC(=O)c1cnc(Nc2ccn(CC(N)=O)n2)s1] | 1H NMR (300 MHz, Methanol-d4) δ 8.02 (s, 1H), 7.76 (s, 1H), 7.57 (s, 1H), 7.36 (s, 1H), 6.91 (d, J = 8.3 Hz, 1H), 6.68 (d, J = 8.2 Hz, 1H), 6.09 (d, J = 1.8 Hz, 1H), 2.16 (s, 3H), 2.11 (s, 3H). MS (ES+) m/z 388.3 (M + H). |
| | 2-((1-(2-amino-2-oxoethyl)-1H-pyrazol-3- | |
| | yl)amino)-N-(3-hydroxy-2,6- | |
| | dimethylphenyl)thiazole-5-carboxamide | |
| BAA-235 | 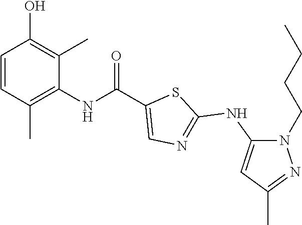 [SMILES: CCCCn1nc(C)cc1Nc1ncc(C(=O)Nc2c(C)ccc(O)c2C)s1] | 1H NMR (300 MHz, Methanol-d4) δ 7.94 (s, 1H), 6.91 (d, J = 8.2 Hz, 1H), 6.68 (d, J = 8.2 Hz, 1H), 6.15 (s, 1H), 4.02 (t, J = 7.1 Hz, 2H), 2.23 (s, 3H), 2.14 (s, 3H), 2.10 (s, 3H), 1.75 (p, J = 7.4 Hz, 2H), 1.30 (dq, J = 14.6, 7.3 Hz, 2H), 0.92 (t, J = 7.4 Hz, 3H). MS (ES+) m/z 400.9 (M + H). |
| | 2-((1-butyl-3-methyl-1H-pyrazol-5- | |
| | yl)amino)-N-(3-hydroxy-2,6- | |
| | dimethylphenyl) thiazole-5-carboxamide | |
| BAA-236 | 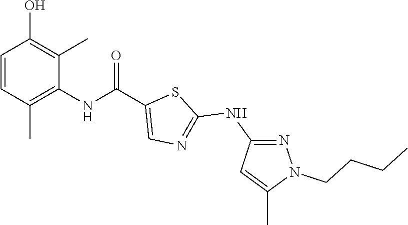 [SMILES: CCCCn1nc(Nc2ncc(C(=O)Nc3c(C)ccc(O)c3C)s2)cc1C] | 1H NMR (300 MHz, Methanol-d4) δ 8.02 (s, 1H), 6.91 (d, J = 8.2 Hz, 1H), 6.68 (d, J = 8.2 Hz, 1H), 5.82 (s, 1H), 4.00 (t, J = 7.0 Hz, 2H), 2.28 (s, 3H), 2.16 (s, 3H), 2.11 (s, 3H), 1.83 (p, J = 7.3 Hz, 2H), 1.41- 1.28 (m, 2H), 0.97 (t, J = 7.4 Hz, 3H). MS (ES+) m/z 400.0 (M + H). |
| | 2-((1-butyl-5-methyl-1H-pyrazol-3- | |
| | yl)amino)-N-(3-hydroxy-2,6- | |
| | dimethylphenyl)thiazole-5-carboxamide | |
| BAA-237 | 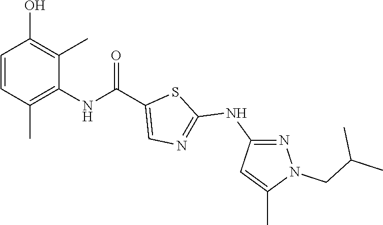 [SMILES: Cc1ccc(O)c(C)c1NC(=O)c1cnc(Nc2cc(C)n(CC(C)C)n2)s1] | 1H NMR (300 MHz, Methanol-d4) δ 8.02 (s, 1H), 6.91 (d, J = 8.2 Hz, 1H), 6.68 (d, J = 8.2 Hz, 1H), 5.81 (s, 1H), 3.79 (d, J = 7.4 Hz, 2H), 2.36-2.22 (m, 4H), 2.16 (s, 3H), 2.11 (s, 3H), 0.93 (d, J = 6.7 Hz, 6H). MS (ES+) m/z 400.0 (M + H). |
| | N-(3-hydroxy-2, 6-dimethylphenyl)-2-((1- | |
| | isobutyl-5-methyl-1H-pyrazol-3- | |
| | yl)amino)thiazole-5-carboxamide | |
| BAA-238 | 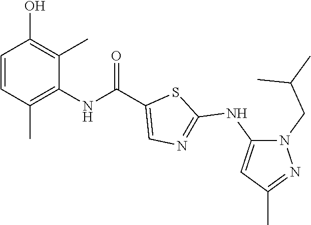 [SMILES: Cc1cc(Nc2ncc(C(=O)Nc3c(C)ccc(O)c3C)s2)n(CC(C)C)n1] | 1H NMR (300 MHz, Methanol-d4) δ 7.94 (s, 1H), 6.91 (d, J = 8.2 Hz, 1H), 6.68 (d, J = 8.2 Hz, 1H), 6.17 (s, 1H), 3.83 (d, J = 7.5 Hz, 2H), 2.24 (s, 3H), 2.14 (s, 3H), 2.10 (s, 3H), 0.88 (d, J = 6.7 Hz, 6H). MS (ES+) m/z 400.0 (M + H). |
| | N-(3-hydroxy-2, 6-dimethylphenyl)-2-((1- | |
| | isobutyl-3-methyl-1H-pyrazol-5- | |
| | yl)amino)thiazole-5-carboxamide | |
| BAA-239 | 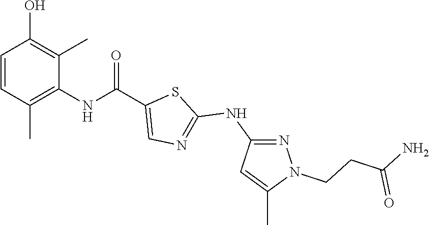 [SMILES: Cc1ccc(O)c(C)c1NC(=O)c1cnc(Nc2cc(C)n(CCC(N)=O)n2)s1] | 1H NMR (300 MHz, Methanol-d4) δ 8.03 (s, 1H), 6.92 (d, J = 8.2 Hz, 1H), 6.69 (d, J = 8.2 Hz, 1H), 5.80- 5.73 (m, 1H), 4.25 (t, J = 6.6 Hz, 2H), 2.84 (t, J = 6.6 Hz, 2H), 2.34- 2.29 (m, 3H), 2.17 (s, 3H), 2.12 (s, 3H). MS (ES+) m/z 415.0 (M + H). |
| | 2-((1-(3-amino-3-oxopropyl)-5-methyl-1H- | |
| | pyrazol-3-yl)amino)-N-(3-hydroxy-2,6- | |
| | dimethylphenyl)thiazole-5-carboxamide | |
| BAA-240 | 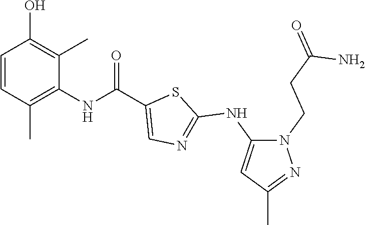 [SMILES: Cc1cc(Nc2ncc(C(=O)Nc3c(C)ccc(O)c3C)s2)n(CCC(N)=O)n1] | 1H NMR (300 MHz, Methanol-d4) δ 7.96 (s, 1H), 6.91 (d, J = 8.2 Hz, 1H), 6.69 (d, J = 8.2 Hz, 1H), 6.17 (s, 1H), 4.28 (t, J = 6.6 Hz, 2H), 2.75 (t, J = 6.6 Hz, 2H), 2.23 (s, 3H), 2.15 (s, 3H), 2.10 (s, 3H). MS (ES+) m/z 415.0 (M + H). |
| | 2-((1-(3-amino-3-oxopropyl)-3-methyl-1H- | |
| | pyrazol-5-yl)amino)-N-(3-hydroxy-2,6- | |
| | dimethylphenyl) thiazole-5-carboxamide | |
| BAA-241 | 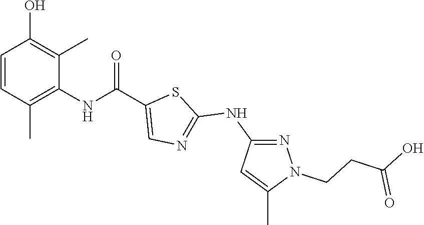 [SMILES: Cc1ccc(O)c(C)c1NC(=O)c1cnc(Nc2cc(C)n(CCC(=O)O)n2)s1] | 1H NMR (300 MHz, Methanol-d4) δ 8.01 (s, 1H), 6.91 (d, J = 8.4 Hz, 1H), 6.68 (d, J = 8.1 Hz, 1H), 5.79 (s, 1H), 4.24 (t, J = 7.1 Hz, 2H), 2.88-2.83 (m, 2H), 2.32 (s, 3H), 2.16 (s, 3H), 2.11 (s, 3H). MS (ES+) m/z 416.3 (M + H). |
| | 3-(3-((5-((3-hydroxy-2,6- | |
| | dimethylphenyl)carbamoyl)thiazol-2- | |
| | yl)amino)-5-methyl-1H-pyrazol-1- | |
| | yl)propanoic acid | |
| BAA-252 | 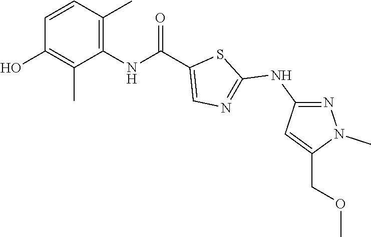 [SMILES: COCc1cc(Nc2ncc(C(=O)Nc3c(C)ccc(O)c3C)s2)nn1C] | ¹H NMR (300 MHz, DMSO-d₆) δ 10.99 (s, 1H), 9.49 (s, 1H), 9.16 (s, 1H), 8.09 (s, 1H), 6.89 (d, J = 8.2 Hz, 1H), 6.69 (d, J = 8.2 Hz, 1H), 6.03 (s, 1H), 4.45 (s, 2H), 3.74 (s, 3H), 3.29 (s, 3H), 2.08 (s, 3H), 2.00 (s, 3H). MS (ES+) m/z 388.0 (M + H)⁺. |
| | N-(3-hydroxy-2,6-dimethylphenyl)-2-((5- | |
| | (methoxymethyl)-1-methyl-1H-pyrazol-3- | |
| | yl)amino)thiazole-5-carboxamide | |
| BAA-253 | 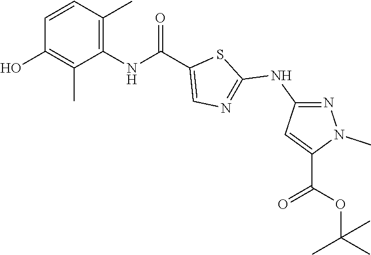 [SMILES: Cc1ccc(O)c(C)c1NC(=O)c1cnc(Nc2cc(C(=O)OC(C)(C)C)n(C)n2)s1] | ¹H NMR (300 MHz, DMSO-d₆) δ 11.14 (s, 1H), 9.54 (s, 1H), 9.17 (s, 1H), 8.11 (s, 1H), 6.90 (d, J = 8.2 Hz, 1H), 6.70 (d, J = 8.2 Hz, 1H), 6.54 (s, 1H), 4.03 (s, 3H), 2.08 (s, 3H), 2.00 (s, 3H), 1.56 (s, 9H). MS (ES+) m/z 444.0 (M + H)⁺. |
| | tert-butyl 3-((5-((3-hydroxy-2,6- | |
| | dimethylphenyl)carbamoyl)thiazol-2- | |
| | yl)amino)-1-methyl-1H-pyrazole-5- | |
| | carboxylate | |
| BAA-254 | 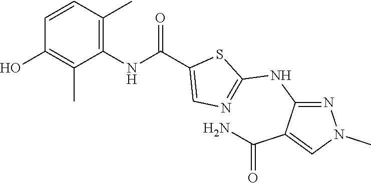 [SMILES: Cc1ccc(O)c(C)c1NC(=O)c1cnc(Nc2nn(C)cc2C(N)=O)s1] | ¹H NMR (300 MHz, DMSO-d₆) δ 10.47 (s, 1H), 9.61 (s, 1H), 9.19 (s, 1H), 8.18 (s, 1H), 8.16 (s, 1H), 7.83 (s, 1H), 7.36 (s, 1H), 6.90 (d, J = 8.2 Hz, 1H), 6.70 (d, J = 8.2 Hz, 1H), 3.87 (s, 3H), 2.08 (s, 3H), 2.00 (s, 3H). MS (ES+) m/z 387.0 (M + H)⁺. |
| | 2-((4-carbamoyl-1-methyl-1H-pyrazol-3- | |
| | yl)amino)-N-(3-hydroxy-2,6- | |
| | dimethylphenyl)thiazole-5-carboxamide | |
| BAA-255 | 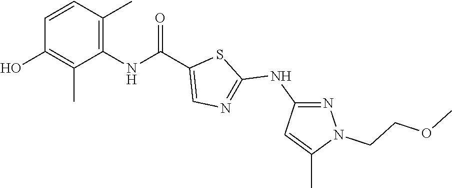 [SMILES: COCCn1nc(Nc2ncc(C(=O)Nc3c(C)ccc(O)c3C)s2)cc1C] | ¹H NMR (300 MHz, DMSO-d₆) δ 10.95 (s, 1H), 9.48 (s, 1H), 9.16 (s, 1H), 8.10 (s, 1H), 6.90 (d, J = 8.2 Hz, 1H), 6.70 (d, J = 8.1 Hz, 1H), 5.78 (s, 1H), 4. 12 (t, J = 5.3 Hz, 2H), 3.69 (t, J = 5.3 Hz, 2H), 3.23 (s, 3H), 2.25 (s, 3H), 2.08 (s, 3H), 2.00 (s, 3H). MS (ES+) m/z 402.5 (M + H)⁺. |
| | N-(3-hydroxy-2, 6-dimethylphenyl)-2-((1- | |
| | (2-methoxyethyl)-5-methyl-1H-pyrazol-3- | |
| | yl)amino)thiazole-5-carboxamide | |
| BAA-256 | 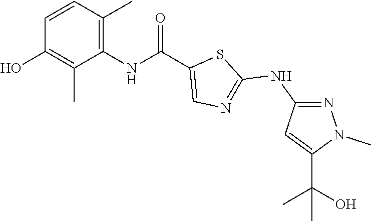 [SMILES: Cc1ccc(O)c(C)c1NC(=O)c1cnc(Nc2cc(C(C)(C)O)n(C)n2)s1] | ¹H NMR (300 MHz, DMSO-d₆) δ 10.93 (s, 1H), 9.48 (s, 1H), 9.18 (s, 1H), 8.09 (s, 1H), 6.89 (d, J = 8.2 Hz, 1H), 6.69 (d, J = 8.2 Hz, 1H), 5.80 (s, 1H), 5.35 (s, 1H), 3.92 (s, 3H), 2.08 (s, 3H), 2.00 (s, 3H), 1.50 (s, 6H). MS (ES+) m/z 402.4 (M + H)⁺. |
| | N-(3-hydroxy-2,6-dimethylphenyl)-2-((5- | |
| | (2-hydroxypropan-2-yl)-1-methyl-1H- | |
| | pyrazol-3-yl)amino)thiazole-5- | |
| | carboxamide | |

### 2-((1-(5-Chloropyridin-2-yl)-4-methyl-1H-pyrazol-3-yl)amino)-N-(3-hydroxy-2,6-dimethylphenyl)thiazole-5-carboxamide( ) BAA-376, 2-((1-(5-chloro-2-fluoropyridin-3-yl)-4-methyl-1H-pyrazol-3-yl)amino)-N-(3-hydroxy-2,6-dimethylphenyl)thiazole-5-carboxamide( ) and 2-((1-(4-bromo-6-chloropyridin-3-yl)-4-methyl-1H-pyrazol-3-yl)amino)-N-(3-hydroxy-2,6-dimethylphenyl)thiazole-5-carboxamide( ) BAA-377

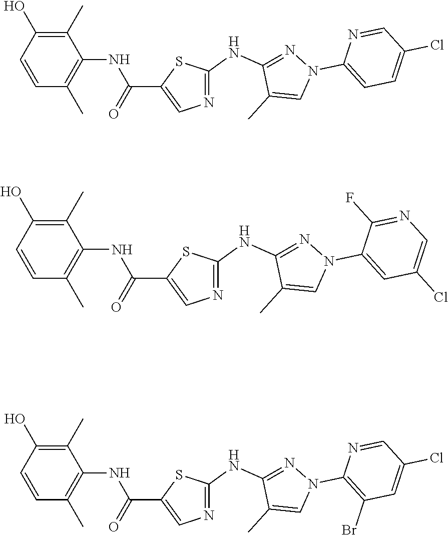 [SMILES: Cc1cn(-c2ccc(Cl)cn2)nc1Nc1ncc(C(=O)Nc2c(C)ccc(O)c2C)s1 ; Cc1cn(-c2cc(Cl)cnc2F)nc1Nc1ncc(C(=O)Nc2c(C)ccc(O)c2C)s1 ; Cc1cn(-c2ncc(Cl)cc2Br)nc1Nc1ncc(C(=O)Nc2c(C)ccc(O)c2C)s1]

Step 1: Using method T, a mixture of 4-methyl-3-nitro-1H-pyrazole (200 mg, 1.57 mmol, 1 eq), copper (I) iodide (60 mg, 0.315 mmol, 0.2 eq) and cesium carbonate (567 mg, 1.73 mmol, 1.1 eq) in DMA (5 mL) was briefly degassed. 3-bromo-5-chloro-2-fluoropyridine (331 mg, 1.57 mmol, 1 eq) was added and the reaction heated to 120° C. for 2 h under microwave irradiation. Ethyl acetate (5 mL) and water (5 mL) were added. The phases were separated and the aqueous phase extracted with ethyl acetate (2×5 mL). Combined organic phases were washed with NH₄Cl (5 mL, 10% w/w aqueous solution), brine (20 mL), dried over MgSO₄, filtered and concentrated in vacuo. Column chromatography (dichloromethane:methanol) gave an inseparable mixture of 5-chloro-2-fluoro-3-(4-methyl-3-nitro-pyrazol-1-yl)pyridine and 3-bromo-5-chloro-2-(4-methyl-3-nitro-pyrazol-1-yl)pyridine (420 mg). This mixture was taken forward without further purification.

Step 2: This mixture was hydrogenated for 16 h in ethanol (16 ml) over palladium on carbon
(173 mg, 1.62 mmol, 0.2 eq) under an atmosphere of hydrogen gas. The reaction mixture was filtered through celite (ethanol) and concentrated in vacuo to give an inseparable mixture of 1-(3-bromo-5-chloro-2-pyridyl)-4-methyl-pyrazol-3-amine, 1-(5-chloro-2-fluoro-3-pyridyl)-4-methyl-pyrazol-3-amine and 1-(5-chloro-2-pyridyl)-4-methyl-pyrazol-3-amine (295 mg) which was used without further purification.

Step 3: Using method D, 2-bromo-N-(3-hydroxy-2,6-dimethyl-phenyl)thiazole-5-carboxamide (200 mg, 0.611 mmol, 1 eq), tris(dibenzylideneacetone) dipalladium (0)(84 mg, 0.0917 mmol, 0.15 eq), cesium carbonate (421 mg, 1.28 mmol, 2.1 eq) and a mixture of 1-(5-chloro-2-pyridyl)-4-methyl-pyrazol-3-amine, 1-(5-chloro-2-fluoro-3-pyridyl)-4-methyl-pyrazol-3-amine and 1-(3-bromo-5-chloro-2-pyridyl)-4-methyl-pyrazol-3-amine (295 mg) in 1,4-dioxane (4 mL) and water
(1 mL) was degassed under nitrogen. 4,5-bis(diphenylphospheno)-9,9-dimethylxanthene (88 mg, 0.153 mmol, 0.25 eq) was added and the reaction heated to 90° C. for 18 h. The reaction was partitioned between EtOAc and water. The organic phase was concentrated in vacuo. Column chromatography (CH2Cl2: methanol) followed by preparative HPLC(water:acetonitrile) gave 2-[1-(5-chloro-2-pyridyl)-4-methyl-pyrazol-3-yl]amino]-N-(3-hydroxy-2,6-dimethyl-phenyl)thiazole-5-carboxamide (0.80 mg, 0.00176 mmol, 0.29%). MS (ES+) m/z 455,457 (M+H). ¹H NMR (300 MHz, MeOD) δ 8.43 (s, 1H), 8.38-8.28 (m, 2H), 8.17-8.03 (m, 1H), 8.00-7.87 (m, 1H), 6.93 (d, J=8.3 Hz, 1H), 6.70 (d, J=8.2 Hz, 1H), 2.19 (d, J=1.2 Hz, 6H), 2.14 (s, 3H). Additionally, 2-[1-(5-chloro-2-fluoro-3-pyridyl)-4-methyl-pyrazol-3-yl]amino]-N-(3-hydroxy-2,6-dimethyl-phenyl)thiazole-5-carboxamide (2.4 mg, 0.00507 mmol, 0.83%). MS (ES+) m/z 473, 475 (M+H). ¹H NMR (300 MHz, MeOD) δ 8.29-8.19 (m, 2H), 8.10 (s, 1H), 7.97-7.88 (m, 1H), 6.92 (d, J=8.2 Hz, 1H), 6.69 (d, J=8.2 Hz, 1H), 2.21-2.16 (m, 6H), 2.12 (s, 3H). ¹⁹F NMR (282 MHz, MeOD) δ 124.97. Additionally, 2-[[1-(3-bromo-5-chloro-2-pyridyl)-4-methyl-pyrazol-3-yl]amino]-N-(3-hydroxy-2,6-dimethyl-phenyl)thiazole-5-carboxamide (3.8 mg, 0.00712 mmol, 1.2%). MS (ES+) m/z 533, 535 (M+H). ¹H NMR (300 MHz, MeOD) δ 8.34 (s, 1H), 8.18-8.12 (m, 1H), 8.05 (s, 1H), 7.99 (s, 1H), 6.89 (d, J=8.2 Hz, 1H), 6.68 (d, J=8.2 Hz, 1H), 2.20-2.14 (m, 6H), 2.12 (s, 3H).

### 2-(6-((5-((3-Hydroxy-2,6-dimethylphenyl)carbamoyl)thiazol-2-yl)amino)pyridin-2-yl) acetic acid (BAA-610)

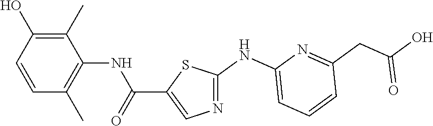 [SMILES: Cc1ccc(O)c(C)c1NC(=O)c1cnc(Nc2cccc(CC(=O)O)n2)s1]

2-Bromo-N-(3-hydroxy-2,6-dimethyl-phenyl)thiazole-5-carboxamide (350 mg, 1.07 mmol, 1 eq), tris(dibenzylideneacetone) dipalladium (0)(147 mg, 0.160 mmol, 0.15 eq), cesium carbonate
(736 mg, 2.25 mmol, 2.1 eq) and ethyl 2-(6-aminopyridin-2-yl)acetate (231 mg, 1.28 mmol, 1.2 eq) in 1,4-dioxane (8 mL) and water (2 mL) was degassed under nitrogen. 4,5-bis(diphenylphospheno)-9,9-dimethylxanthene (155 mg, 0.267 mmol, 0.250 eq) was added and the reaction degassed again, then heated to 90° C. for 18 h. The reaction was partitioned between EtOAc and water. The aqueous phase was acidified to pH=3 with 2 M HCl and extracted with ethyl acetate (2×10 mL). The organic phase was concentrated in vacuo. Column chromatography (CH2Cl2: methanol) gave 2-[6-[5-[(3-hydroxy-2,6-dimethyl-phenyl) carbamoyl]thiazol-2-yl]amino]-2-pyridyl]acetic acid (264 mg, 0.663 mmol, 62%). MS (ES+) m/z 399 (M+H).
   General Method M: Buchwald Coupling of Bromothiazole with Substituted Amines

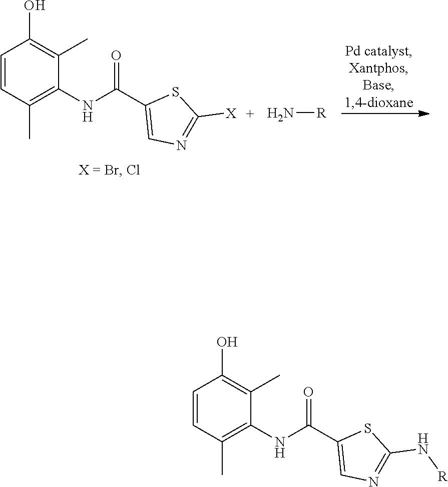

A mixture of bromothiazole (1.0 eq), substituted amine (1.0-1.5 eq), cesium carbonate or potassium carbonate (1.1-5.0 eq), Pd₂dba₃ or Pd(OAc) 2 (10%), Xantphos (20%) and 1,4-dioxane or 1,4-dioxane:water (4:1)(0.1-0.3 M) was degassed with nitrogen and stirred at reflux for 6-24 h. After cooling to rt, the mixture was diluted H₂O and extracted with EOAc (3×). The combined organic phases were dried over MgSO₄, filtered and the solvent was removed under reduced pressure. The crude may be purified by chromatography or other methods if required.

### Representative Example

### N-(3-Hydroxy-2,6-dimethylphenyl)-2-((4-(methylsulfonyl)phenyl)amino)thiazole-5-carboxamide ( ) BAA-275

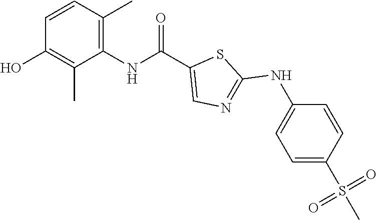 [SMILES: Cc1ccc(O)c(C)c1NC(=O)c1cnc(Nc2ccc(S(C)(=O)=O)cc2)s1]

A mixture of 4-(methylsulfonyl) aniline (45 mg, 0.263 mmol, 1.00 eq), 2-bromo-N-(3-hydroxy-2,6-dimethyl-phenyl)thiazole-5-carboxamide (86 mg, 0.263 mmol, 1.00 eq), cesium carbonate (172 mg, 0.526 mmol, 2.00 eq), tris(dibenzylideneacetone) dipalladium (0)(24 mg, 0.0263 mmol, 0.100 eq), 4,5-bis(diphenylphospheno)-9,9-dimethylxanthene (30 mg, 0.0526 mmol, 0.200 eq) and 1,4-dioxane (2.5 mL) was degassed with nitrogen and stirred at 95° C. for 4 h. After cooling to rt, the mixture was diluted with 0.5 M NaOH(10 mL) and washed with DCM (2×10 mL). The pH of the aqueous phase was adjusted to ~7 and extracted with EtOAc (3×30 mL). The combined organic phases were dried over MgSO4, filtered and the solvent was removed under reduced pressure. The crude was purified by prep HPLC(low pH) to afford N-(3-hydroxy-2,6-dimethyl-phenyl)-2-(4-methylsulfonylanilino)thiazole-5-carboxamide (78 mg, 0.186 mmol, 71%) as an off white solid. ¹H NMR (300 MHz, Methanol-d4/chloroform -d) δ 8.42-8.38 (m, 1H), 8.06 (s, 1H), 7.85-7.76 (m, 1H), 7.57-7.51 (m, 2H), 6.89 (d, J=8.5 Hz, 1H), 6.69 (d, J=8.2 Hz, 1H), 3.13 (s, 3H), 2.15 (s, 3H), 2.12 (s, 3H). MS (ES+) m/z 418.2 (M+H)⁺.

The following example compounds were prepared similarly using Method M using the appropriately substituted amine.

| # | Structure & Name | Analytical Data |
|---|---|---|
| BAA- 390 | 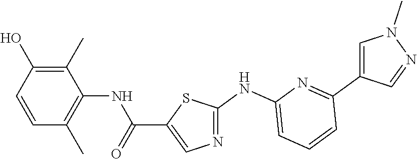 [SMILES: Cc1ccc(O)c(C)c1NC(=O)c1cnc(Nc2cccc(-c3cnn(C)c3)n2)s1] N-(3-hydroxy-2,6-dimethylphenyl)-2- ((6-(1-methyl-1H-pyrazol-4-yl)pyridin- 2-yl)amino)thiazole-5-carboxamide | MS (ES+) m/z 421 (M + H). ¹H NMR (300 MHz, DMSO) δ 11.66 (s, 1H), 9.58 (s, 1H), 9.16 (d, J = 0.7 Hz, 1H), 8.32 (s, 1H), 8.24 (d, J = 1.0 Hz, 1H), 8.14 (d, J = 0.8 Hz, 1H), 7.73 (t, J = 7.9 Hz, 1H), 7.31-7.22 (m, 1H), 6.90 (dd, J = 8.2, 1.2 Hz, 2H), 6.75- 6.65 (m, 1H), 3.90 (s, 3H), 2.10 (s, 3H), 2.02 (s, 3H). |
| BAA- 396 | 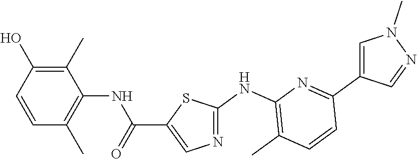 [SMILES: Cc1ccc(-c2cnn(C)c2)nc1Nc1ncc(C(=O)Nc2c(C)ccc(O)c2C)s1] N-(3-hydroxy-2,6-dimethylphenyl)-2- ((3-methyl-6-(1-methyl-1H-pyrazol-4- yl)pyridin-2-yl)amino)thiazole-5- carboxamide | MS (ES+) m/z 435 (M + H). ¹H NMR (300 MHz, MeOD) δ 8.30 (s, 1H), 8.21 (s, 1H), 8.08 (d, J = 0.7 Hz, 1H), 7.55 (dd, J = 7.6, 0.9 Hz, 1H), 7.22 (d, J = 7.6 Hz, 1H), 6.98-6.89 (m, 1H), 6.71 (d, J = 8.2 Hz, 1H), 3.96 (s, 3H), 2.37 (s, 3H), 2.23-2.18 (m, 3H), 2.16 (s, 3H). |
| BAA- 397 | 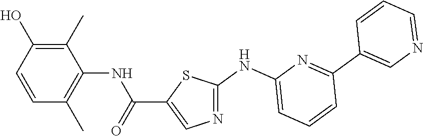 [SMILES: Cc1ccc(O)c(C)c1NC(=O)c1cnc(Nc2cccc(-c3cccnc3)n2)s1] 2-([2,3′-bipyridin]-6-ylamino)-N-(3- hydroxy-2,6-dimethylphenyl)thiazole- 5-carboxamide | MS (ES+) m/z 418 (M + H). |
| BAA- 399 | 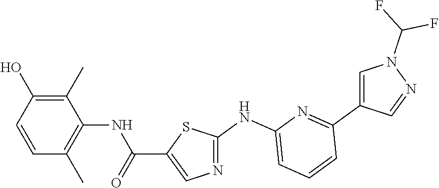 [SMILES: Cc1ccc(O)c(C)c1NC(=O)c1cnc(Nc2cccc(-c3cnn(C(F)F)c3)n2)s1] 2-((6-(1-(difluoromethyl)-1H-pyrazol-4- yl)pyridin-2-yl)amino)-N-(3-hydroxy- 2,6-dimethylphenyl)thiazole-5- carboxamide | MS (ES+) m/z 457 (M + H). ¹H NMR (300 MHz, MeOD) δ 8.68 (s, 1H), 8.28 (s, 1H), 8.13 (d, J = 1.7 Hz, 1H), 7.74- 7.62 (m, 1H), 7.45 (t, J = 57.7 Hz, 1H), 7.29- 7.20 (m, 1H), 6.90 (d, J = 7.9 Hz, 2H), 6.69 (d, J = 8.2 Hz, 1H), 2.18 (s, 3H), 2.14 (s, 3H). ¹⁹F NMR (282 MHz, MeOD) δ 95.31 (d, J = 8.9 Hz). |
| BAA- 400 | 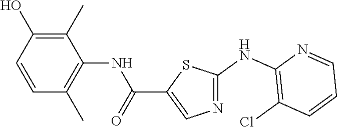 [SMILES: Cc1ccc(O)c(C)c1NC(=O)c1cnc(Nc2ncccc2Cl)s1] 2-((3-chloropyridin-2-yl)amino)-N-(3- hydroxy-2,6-dimethylphenyl)thiazole- 5-carboxamide | MS (ES+) m/z 375, 377 (M + H). ¹H NMR (300 MHz, MeOD) δ 8.30 (d, J = 4.8 Hz, 1H), 8.15 (d, J = 1.8 Hz, 1H), 7.77 (d, J = 7.8 Hz, 1H), 6.99 (dd, J = 7.9, 4.9 Hz, 1H), 6.90 (d, J = 8.2 Hz, 1H), 6.69 (d, J = 8.2 Hz, 1H), 2.17 (s, 3H), 2.13 (s, 3H). |
| BAA- 401 | 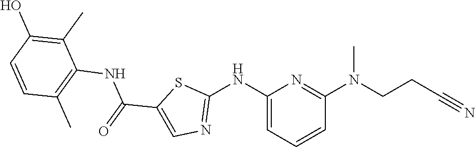 [SMILES: Cc1ccc(O)c(C)c1NC(=O)c1cnc(Nc2cccc(N(C)CCC#N)n2)s1] 2-((6-((2- cyanoethyl)(methyl)amino)pyridin-2- yl)amino)-N-(3-hydroxy-2,6- dimethylphenyl)thiazole-5- carboxamide | MS (ES+) m/z 423 (M + H). ¹H NMR (300 MHz, MeOD) δ 8.10 (d, J = 1.4 Hz, 1H), 7.49 (dd, J = 8.5, 7.2 Hz, 1H), 6.90 (d, J = 8.4 Hz, 1H), 6.68 (d, J = 8.2 Hz, 1H), 6.31 (dd, J = 7.7, 1.3 Hz, 1H), 6.19 (d, J = 8.3 Hz, 1H), 4.11 (t, J = 6.0 Hz, 2H), 3.20 (d, J = 1.4 Hz, 3H), 2.85 (t, J = 6.1 Hz, 2H), 2.16 (s, 3H), 2.12 (s, 3H). |
| BAA- 402 | 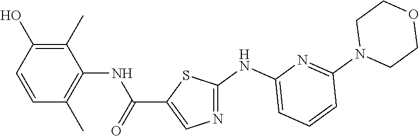 [SMILES: Cc1ccc(O)c(C)c1NC(=O)c1cnc(Nc2cccc(N3CCOCC3)n2)s1] N-(3-hydroxy-2,6-dimethylphenyl)-2- ((4-morpholinopyridin-2- yl) amino)thiazole-5-carboxamide | MS (ES+) m/z 426 (M + H). ¹H NMR (300 MHz, MeOD) δ 8.12 (s, 1H), 8.06 (d, J = 6.2 Hz, 1H), 6.99-6.90 (m, 1H), 6.71 (d, J = 8.2 Hz, 1H), 6.60 (dd, J = 6.2, 2.3 Hz, 1H), 6.43 (d, J = 2.2 Hz, 1H), 3.89-3.79 (m, 4H), 3.40-3.34 (m, 4H), 2.19 (s, 3H), 2.14 (s, 3H). |
| BAA- 403 | 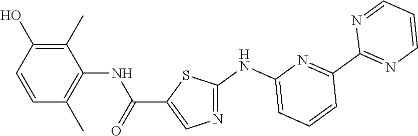 [SMILES: Cc1ccc(O)c(C)c1NC(=O)c1cnc(Nc2cccc(-c3ncccn3)n2)s1] N-(3-hydroxy-2,6-dimethylphenyl)-2- ((6-(pyrimidin-2-yl)pyridin-2- yl)amino)thiazole-5-carboxamide | MS (ES+) m/z 419 (M + H). ¹H NMR (300 MHz, MeOD) δ 9.55 (s, 2H), 9.18 (s, 1H), 8.17 (s, 1H), 7.86 (t, J = 7.9 Hz, 1H), 7.58 (s, 1H), 7.14 (d, J = 8.2 Hz, 1H), 6.91 (d, J = 8.6 Hz, 1H), 6.69 (d, J = 8.2 Hz, 1H), 2.20-2.15 (m, 6H). |
| BAA- 404 | 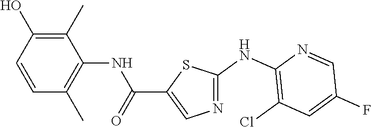 [SMILES: Cc1ccc(O)c(C)c1NC(=O)c1cnc(Nc2ncc(F)cc2Cl)s1] 2-((3-chloro-5-fluoropyridin-2- yl)amino)-N-(3-hydroxy-2,6- dimethylphenyl)thiazole-5- carboxamide | MS (ES+) m/z 393, 395 (M + H). ¹H NMR (300 MHz, MeOD) δ 8.23 (d, J = 2.5 Hz, 1H), 8.14 (s, 1H), 7.75-7.66 (m, 1H), 6.90 (d, J = 8.2 Hz, 1H), 6.68 (d, J = 8.2 Hz, 1H), 2.16 (s, 3H), 2.12 (s, 3H). ¹⁹F NMR (282 MHz, MeOD) δ 135.01. |
| BAA- 405 | 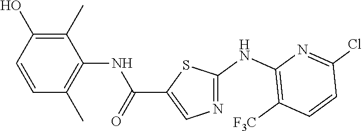 [SMILES: Cc1ccc(O)c(C)c1NC(=O)c1cnc(Nc2nc(Cl)ccc2C(F)(F)F)s1] 2-((6-chloro-3-(trifluoromethyl)pyridin- 2-yl)amino)-N-(3-hydroxy-2,6- dimethylphenyl)thiazole-5- carboxamide | MS (ES+) m/z 443, 445 (M + H). ¹H NMR (300 MHz, MeOD) δ 8.28-7.86 (m, 2H), 7.09 (s, 1H), 6.97-6.88 (m, 1H), 6.70 (d, J = 8.2 Hz, 1H), 2.17 (d, J = 0.7 Hz, 3H), 2.12 (s, 3H). ¹⁹F NMR (282 MHz, MeOD) δ 63.51. |
| BAA- 406 | 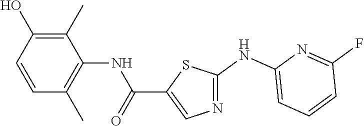 [SMILES: Cc1ccc(O)c(C)c1NC(=O)c1cnc(Nc2cccc(F)n2)s1] 2-((6-fluoropyridin-2-yl)amino)-N-(3- hydroxy-2,6-dimethylphenyl)thiazole- 5-carboxamide | MS (ES+) m/z 359 (M + H). ¹H NMR (300 MHz, MeOD) δ 8.13 (s, 1H), 7.77 (q, J = 8.0 Hz, 1H), 6.90 (dd, J = 8.1, 2.2 Hz, 2H), 6.68 (d, J = 8.2 Hz, 1H), 6.54 (dd, J = 7.9, 2.3 Hz, 1H), 2.16 (s, 3H), 2.12 (s, 3H). ¹⁹F NMR (282 MHz, MeOD) δ 71.41 (t, J = 4.4 Hz). |
| BAA- 407 | 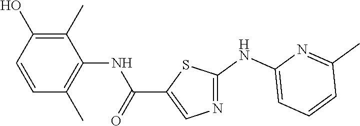 [SMILES: Cc1cccc(Nc2ncc(C(=O)Nc3c(C)ccc(O)c3C)s2)n1] N-(3-hydroxy-2,6-dimethylphenyl)-2- ((6-methylpyridin-2-yl)amino)thiazole- 5-carboxamide | MS (ES+) m/z 355 (M + H). ¹H NMR (300 MHz, MeOD) δ 8.11 (d, J = 1.5 Hz, 1H), 7.55 (t, J = 7.8 Hz, 1H), 6.90 (d, J = 8.3 Hz, 1H), 6.80 (dd, J = 7.7, 3.2 Hz, 2H), 6.68 (d, J = 8.2 Hz, 1H), 2.53 (s, 3H), 2.17 (s, 3H), 2.13 (s, 3H). |
| BAA- 408 | 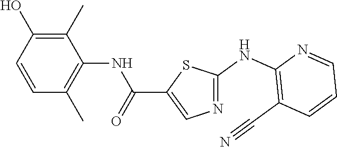 [SMILES: Cc1ccc(O)c(C)c1NC(=O)c1cnc(Nc2ncccc2C#N)s1] 2-((3-cyanopyridin-2-yl)amino)-N-(3- hydroxy-2,6-dimethylphenyl)thiazole- 5-carboxamide | MS (ES+) m/z 366 (M + H). ¹H NMR (300 MHz, MeOD) δ 8.83 (s, 1H), 8.73 (d, J = 4.6 Hz, 1H), 8.68 (dd, J = 8.0, 1.8 Hz, 1H), 7.43 (dd, J = 8.1, 4.7 Hz, 1H), 6.94 (d, J = 8.2 Hz, 1H), 6.71 (d, J = 8.2 Hz, 1H), 2.18 (s, 3H), 2.13 (s, 3H). |
| BAA- 409 | 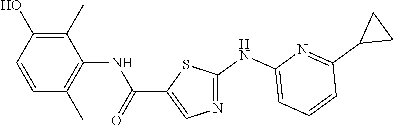 [SMILES: Cc1ccc(O)c(C)c1NC(=O)c1cnc(Nc2cccc(C3CC3)n2)s1] 2-((6-cyclopropylpyridin-2-yl)amino)- N-(3-hydroxy-2,6- dimethylphenyl)thiazole-5- carboxamide | MS (ES+) m/z 381 (M + H). ¹H NMR (300 MHz, MeOD) δ 7.98 (d, J = 7.2 Hz, 1H), 7.45 (s, 1H), 7.35 (s, 1H), 6.88 (d, J = 8.5 Hz, 1H), 6.81 (d, J = 7.3 Hz, 1H), 6.67 (d, J = 8.5 Hz, 2H), 2.15 (s, 3H), 2.11 (t, J = 3.1 Hz, 3H), 1.98 (s, 1H), 1.30 (s, 2H), 0.94 (d, J = 7.7 Hz, 2H). |
| BAA- 410 | 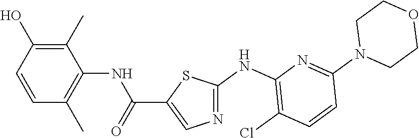 [SMILES: Cc1ccc(O)c(C)c1NC(=O)c1cnc(Nc2nc(N3CCOCC3)ccc2Cl)s1] 2-((3-chloro-6-morpholinopyridin-2- yl)amino)-N-(3-hydroxy-2,6- dimethylphenyl)thiazole-5- carboxamide | MS (ES+) m/z 460, 462 (M + H). ¹H NMR (300 MHz, MeOD) δ 8.17 (s, 1H), 7.57 (d, J = 8.8 Hz, 1H), 6.92 (dt, J = 8.2, 0.7 Hz, 1H), 6.69 (d, J = 8.2 Hz, 1H), 6.43 (d, J = 8.8 Hz, 1H), 3.82 (dd, J = 5.8, 4.0 Hz, 4H), 3.62 (dd, J = 5.8, 4.0 Hz, 4H), 2.17 (d, J = 0.6 Hz, 3H), 2.12 (s, 3H). |
| BAA- 411 | 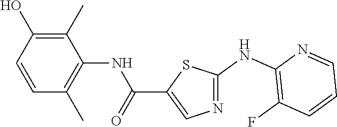 [SMILES: Cc1ccc(O)c(C)c1NC(=O)c1cnc(Nc2ncccc2F)s1] 2-((3-fluoropyridin-2-yl)amino)-N-(3- hydroxy-2,6-dimethylphenyl)thiazole- 5-carboxamide | MS (ES+) m/z 359 (M + H). ¹H NMR (300 MHz, MeOD) δ 8.18 (d, J = 7.5 Hz, 2H), 7.59-7.46 (m, 1H), 7.00 (dt, J = 8.3, 4.1 Hz, 1H), 6.91 (d, J = 8.2 Hz, 1H), 6.69 (d, J = 8.2 Hz, 1H), 2.17 (s, 3H), 2.13 (s, 3H). ¹⁹F NMR (282 MHz, MeOD) δ 138.38. |
| BAA- 412 | 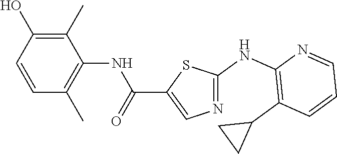 [SMILES: Cc1ccc(O)c(C)c1NC(=O)c1cnc(Nc2ncccc2C2CC2)s1] 2-((3-cyclopropylpyridin-2-yl)amino)- N-(3-hydroxy-2,6- dimethylphenyl)thiazole-5- carboxamide | MS (ES+) m/z 381 (M + H). ¹H NMR (300 MHz, MeOD) δ 8.24 (ddd, J = 5.0, 1.7, 0.7 Hz, 1H), 8.18 (s, 1H), 7.54 (ddd, J = 7.5, 1.7, 0.9 Hz, 1H), 6.99-6.90 (m, 2H), 6.69 (d, J = 8.2 Hz, 1H), 2.20-2.16 (m, 3H), 2.13 (s, 3H), 1.96 (td, J = 8.6, 4.4 Hz, 1H), 1.17-1.06 (m, 2H), 0.76-0.66 (m, 2H). |
| BAA- 413 | 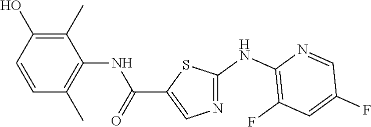 [SMILES: Cc1ccc(O)c(C)c1NC(=O)c1cnc(Nc2ncc(F)cc2F)s1] 2-((3,5-difluoropyridin-2-yl)amino)-N- (3-hydroxy-2,6- dimethylphenyl)thiazole-5- carboxamide | MS (ES+) m/z 375 (M + H). ¹H NMR (300 MHz, MeOD) δ 8.20 (d, J = 2.4 Hz, 1H), 8.17 (s, 1H), 7.67 (ddd, J = 10.5, 8.1, 2.5 Hz, 1H), 6.92 (dt, J = 8.2, 0.7 Hz, 1H), 6.69 (d, J = 8.2 Hz, 1H), 2.17 (d, J = 0.7 Hz, 3H), 2.12 (s, 3H). ¹⁹F NMR (282 MHz, MeOD) δ 133.67, 135.79 (d, J = 1.9 Hz). |
| BAA- 414 | 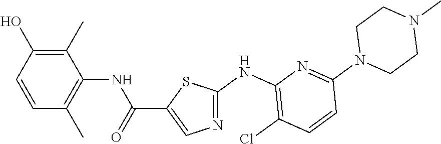 [SMILES: Cc1ccc(O)c(C)c1NC(=O)c1cnc(Nc2nc(N3CCN(C)CC3)ccc2Cl)s1] 2-((3-chloro-6-(4-methylpiperazin-1- yl)pyridin-2-yl)amino)-N-(3-hydroxy- 2,6-dimethylphenyl)thiazole-5- carboxamide | MS (ES+) m/z 473, 471 (M + H). ¹H NMR (300 MHz, MeOD) δ 8.18 (s, 1H), 7.62-7.53 (m, 1H), 6.93 (d, J = 8.3 Hz, 1H), 6.70 (d, J = 8.3 Hz, 1H), 6.46 (d, J = 8.9 Hz, 1H), 3.71 (t, J = 5.2 Hz, 4H), 2.60 (t, J = 5.1 Hz, 4H), 2.34 (s, 3H), 2.18 (s, 3H), 2.13 (s, 3H). |
| BAA- 415 | 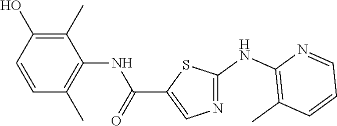 [SMILES: Cc1cccnc1Nc1ncc(C(=O)Nc2c(C)ccc(O)c2C)s1] N-(3-hydroxy-2,6-dimethylphenyl)-2- ((3-methylpyridin-2-yl)amino)thiazole- 5-carboxamide | MS (ES+) m/z 355 (M + H). ¹H NMR (300 MHz, MeOD) δ 8.24 (ddd, J = 5.0, 1.8, 0.8 Hz, 1H), 8.17 (s, 1H), 7.58 (ddd, J = 7.4, 1.8, 0.9 Hz, 1H), 6.98-6.88 (m, 2H), 6.69 (d, J = 8.2 Hz, 1H), 2.39 (s, 3H), 2.18 (d, J = 0.7 Hz, 3H), 2.13 (s, 3H). |
| BAA- 416 | 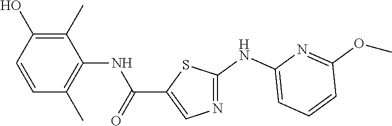 [SMILES: COc1cccc(Nc2ncc(C(=O)Nc3c(C)ccc(O)c3C)s2)n1] N-(3-hydroxy-2,6-dimethylphenyl)-2- ((6-methoxypyridin-2- yl)amino)thiazole-5-carboxamide | MS (ES+) m/z 371 (M + H). ¹H NMR (300 MHz, MeOD) δ 8.10 (d, J = 1.5 Hz, 1H), 7.55 (t, J = 7.9 Hz, 1H), 6.90 (d, J = 8.2 Hz, 1H), 6.68 (d, J = 8.2 Hz, 1H), 6.54 (d, J = 7.8 Hz, 1H), 6.35 (d, J = 8.0 Hz, 1H), 4.10 (s, 3H), 2.16 (s, 3H), 2.12 (s, 3H). |
| BAA- 417 | 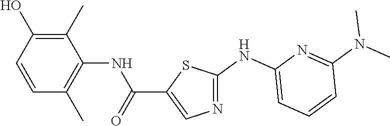 [SMILES: Cc1ccc(O)c(C)c1NC(=O)c1cnc(Nc2cccc(N(C)C)n2)s1] 2-((6-(dimethylamino)pyridin-2- yl)amino)-N-(3-hydroxy-2,6- dimethylphenyl)thiazole-5- carboxamide | MS (ES+) m/z 382 (M + H). ¹H NMR (300 MHz, MeOD) δ 8.08 (s, 1H), 7.41 (t, J = 8.0 Hz, 1H), 6.94-6.87 (m, 1H), 6.68 (d, J = 8.3 Hz, 1H), 6.20 (dd, J = 7.9, 2.4 Hz, 1H), 6.12 (d, J = 8.6 Hz, 1H), 3.18 (d, J = 2.9 Hz, 6H), 2.16 (d, J = 2.6 Hz, 3H), 2.12 (d, J = 2.6 Hz, 3H). |
| BAA- 418 | 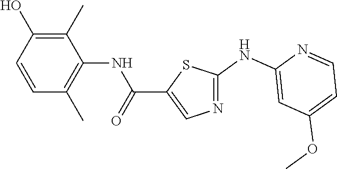 [SMILES: COc1ccnc(Nc2ncc(C(=O)Nc3c(C)ccc(O)c3C)s2)c1] N-(3-hydroxy-2,6-dimethylphenyl)-2- ((4-methoxypyridin-2- yl)amino)thiazole-5-carboxamide | MS (ES+) m/z 371 (M + H). ¹H NMR (300 MHz, MeOD) δ 8.17 (dd, J = 5.8, 0.6 Hz, 1H), 8.12 (s, 1H), 6.96-6.88 (m, 1H), 6.69 (d, J = 8.2 Hz, 1H), 6.63-6.55 (m, 2H), 3.88 (s, 3H), 2.19-2.16 (m, 3H), 2.12 (s, 3H). |
| BAA- 419 | 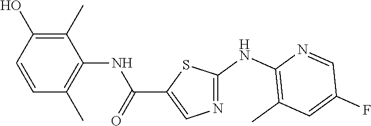 [SMILES: Cc1cc(F)cnc1Nc1ncc(C(=O)Nc2c(C)ccc(O)c2C)s1] 2-((5-fluoro-3-methylpyridin-2- yl)amino)-N-(3-hydroxy-2,6- dimethylphenyl)thiazole-5- carboxamide | MS (ES+) m/z 373 (M + H). ¹H NMR (300 MHz, MeOD) δ 8.16 (s, 1H), 8.14 (dd, J = 2.8, 0.8 Hz, 1H), 7.54-7.44 (m, 1H), 6.92 (d, J = 8.2 Hz, 1H), 6.69 (d, J = 8.2 Hz, 1H), 2.41 (t, J = 0.8 Hz, 3H), 2.18-2.16 (m, 3H), 2.12 (s, 3H). ¹⁹F NMR (282 MHz, MeOD) δ 139.50. |
| BAA- 420 | 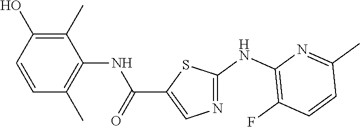 [SMILES: Cc1ccc(F)c(Nc2ncc(C(=O)Nc3c(C)ccc(O)c3C)s2)n1] 2-((3-fluoro-6-methylpyridin-2- yl)amino)-N-(3-hydroxy-2,6- dimethylphenyl)thiazole-5- carboxamide | MS (ES+) m/z 373 (M + H). ¹H NMR (300 MHz, MeOD) δ 8.18 (s, 1H), 7.45 (dd, J = 10.8, 8.1 Hz, 1H), 6.93 (dt, J = 8.3, 0.7 Hz, 1H), 6.86 (ddd, J = 8.1, 3.0, 0.7 Hz, 1H), 6.69 (d, J = 8.2 Hz, 1H), 2.54 (t, J = 0.8 Hz, 3H), 2.18 (d, J = 0.6 Hz, 3H), 2.13 (s, 3H). ¹⁹F NMR (282 MHz, MeOD) δ 144.52. |
| BAA- 421 | 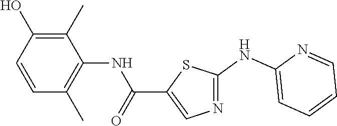 [SMILES: Cc1ccc(O)c(C)c1NC(=O)c1cnc(Nc2ccccn2)s1] N-(3-hydroxy-2,6-dimethylphenyl)-2- (pyridin-2-ylamino)thiazole-5- carboxamide | MS (ES+) m/z 339 (M + H). ¹H NMR (300 MHz, MeOD) δ 8.37 (ddd, J = 5.1, 1.9, 0.9 Hz, 1H), 8.14 (s, 1H), 7.73 (ddd, J = 8.4, 7.3, 1.9 Hz, 1H), 7.06 (dt, J = 8.3, 0.9 Hz, 1H), 6.99 (ddd, J = 7.3, 5.1, 1.0 Hz, 1H), 6.92 (dt, J = 8.2, 0.7 Hz, 1H), 6.69 (d, J = 8.2 Hz, 1H), 2.20-2.16 (m, 3H), 2.12 (s, 3H). |
| BAA- 422 | 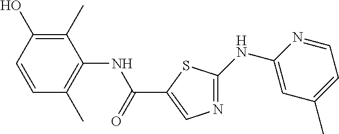 [SMILES: Cc1ccnc(Nc2ncc(C(=O)Nc3c(C)ccc(O)c3C)s2)c1] N-(3-hydroxy-2,6-dimethylphenyl)-2- ((4-methylpyridin-2-yl)amino)thiazole- 5-carboxamide | MS (ES+) m/z 355 (M + H). ¹H NMR (300 MHz, MeOD) δ 8.22 (dd, J = 5.2, 0.8 Hz, 1H), 8.13 (s, 1H), 6.95-6.80 (m, 3H), 6.69 (d, J = 8.2 Hz, 1H), 2.36 (d, J = 0.8 Hz, 3H), 2.17 (d, J = 0.7 Hz, 3H), 2.12 (s, 3H). |
| BAA- 423 | 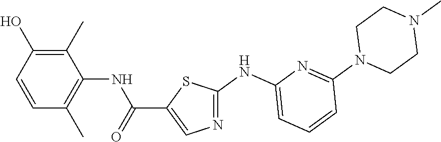 [SMILES: Cc1ccc(O)c(C)c1NC(=O)c1cnc(Nc2cccc(N3CCN(C)CC3)n2)s1] N-(3-hydroxy-2,6-dimethylphenyl)-2- ((6-(4-methylpiperazin-1-yl)pyridin-2- yl)amino)thiazole-5-carboxamide | MS (ES+) m/z 439 (M + H). ¹H NMR (300 MHz, MeOD) δ 8.13 (s, 1H), 7.56-7.44 (m, 1H), 6.92 (d, J = 8.3 Hz, 1H), 6.69 (d, J = 8.2 Hz, 1H), 6.36 (t, J = 8.2 Hz, 2H), 3.71 (t, J = 5.1 Hz, 4H), 2.61 (t, J = 5.1 Hz, 4H), 2.35 (s, 3H), 2.17 (s, 3H), 2.13 (s, 3H). |
| BAA- 424 | 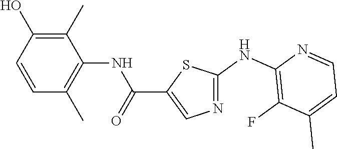 [SMILES: Cc1ccnc(Nc2ncc(C(=O)Nc3c(C)ccc(O)c3C)s2)c1F] 2-((3-fluoro-4-methylpyridin-2- yl)amino)-N-(3-hydroxy-2,6- dimethylphenyl)thiazole-5- carboxamide | MS (ES+) m/z 373 (M + H). ¹H NMR (300 MHz, MeOD) δ 8.17 (s, 1H), 8.04 (d, J = 5.0 Hz, 1H), 6.95-6.87 (m, 2H), 6.69 (d, J = 8.2 Hz, 1H), 2.35 (d, J = 2.0 Hz, 3H), 2.19-2.15 (m, 3H), 2.12 (s, 3H). ¹⁹F NMR (282 MHz, MeOD) δ 145.39 |
| BAA- 425 | 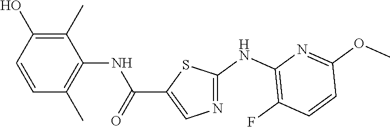 [SMILES: COc1ccc(F)c(Nc2ncc(C(=O)Nc3c(C)ccc(O)c3C)s2)n1] 2-((3-fluoro-6-methoxypyridin-2- yl)amino)-N-(3-hydroxy-2,6- dimethylphenyl)thiazole-5- carboxamide | MS (ES+) m/z 389 (M + H). ¹H NMR (300 MHz, MeOD) δ 6.60 (s, 1H), 5.95 (dd, J = 10.0, 8.7 Hz, 1H), 5.35 (d, J = 8.3 Hz, 1H), 5.12 (d, J = 8.2 Hz, 1H), 4.80 (dd, J = 8.7, 2.4 Hz, 1H), 2.53 (s, 3H), 0.60 (s, 3H), 0.55 (s, 3H). ¹⁹F NMR (282 MHz, MeOD) δ 152.14. |
| BAA- 429 | 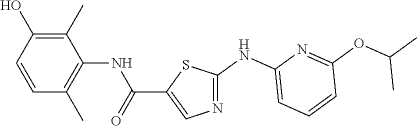 [SMILES: Cc1ccc(O)c(C)c1NC(=O)c1cnc(Nc2cccc(OC(C)C)n2)s1] N-(3-hydroxy-2,6-dimethylphenyl)-2- ((6-isopropoxypyridin-2- yl)amino)thiazole-5-carboxamide | MS (ES+) m/z 399 (M + H). ¹H NMR (300 MHz, MeOD) δ 8.14 (s, 1H), 7.58 (t, J = 8.0 Hz, 1H), 7.01-6.88 (m, 1H), 6.69 (d, J = 8.2 Hz, 1H), 6.55 (dd, J = 7.8, 0.7 Hz, 1H), 6.32 (dd, J = 8.0, 0.7 Hz, 1H), 5.58 (h, J = 6.1 Hz, 1H), 2.18 (s, 3H), 2.13 (s, 3H), 1.39 (d, J = 6.1 Hz, 6H). |
| BAA- 430 | 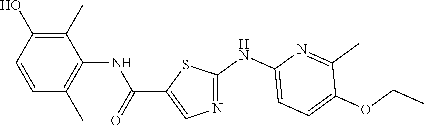 [SMILES: CCOc1ccc(Nc2ncc(C(=O)Nc3c(C)ccc(O)c3C)s2)nc1C] 2-((5-ethoxy-6-methylpyridin-2- yl)amino)-N-(3-hydroxy-2,6- dimethylphenyl)thiazole-5- carboxamide | MS (ES+) m/z 399 (M + H). ¹H NMR (300 MHz, MeOD) δ 8.11 (s, 1H), 7.36 (d, J = 8.7 Hz, 1H), 6.92 (d, J = 8.3 Hz, 1H), 6.90-6.83 (m, 1H), 6.69 (d, J = 8.2 Hz, 1H), 4.06 (q, J = 7.0 Hz, 2H), 2.48 (s, 3H), 2.18 (s, 3H), 2.13 (s, 3H), 1.42 (t, J = 7.0 Hz, 3H). |
| BAA- 431 | 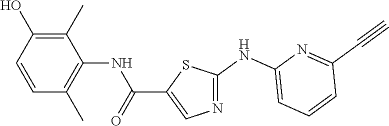 [SMILES: C#Cc1cccc(Nc2ncc(C(=O)Nc3c(C)ccc(O)c3C)s2)n1] 2-((6-ethynylpyridin-2-yl)amino)-N-(3- hydroxy-2,6-dimethylphenyl)thiazole- 5-carboxamide | MS (ES+) m/z 365 (M + H). ¹H NMR (300 MHz, MeOD) δ 8.54 (s, 1H), 7.51 (dd, J = 8.5, 7.3 Hz, 1H), 6.98 (dd, J = 7.3, 0.8 Hz, 1H), 6.96-6.90 (m, 1H), 6.71 (d, J = 8.2 Hz, 1H), 6.67 (dd, J = 8.5, 0.8 Hz, 1H), 2.20-2.14 (m, 3H), 2.12 (s, 3H). |
| BAA- 432 | 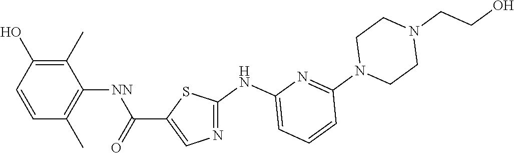 N-(3-hydroxy-2,6-dimethylphenyl)-2- ((6-(4-(2-hydroxyethyl)piperazin-1- yl)pyridin-2-yl)amino)thiazole-5- carboxamide | MS (ES+) m/z 469 (M + H). ¹H NMR (300 MHz, MeOD) δ 8.12 (s, 1H), 7.54-7.44 (m, 1H), 6.94-6.88 (m, 1H), 6.69 (d, J = 8.2 Hz, 1H), 6.35 (dd, J = 9.5, 8.0 Hz, 2H), 3.71 (q, J = 5.6 Hz, 6H), 2.71-2.63 (m, 4H), 2.58 (t, J = 6.0 Hz, 2H), 2.18 (s, 3H), 2.13 (s, 3H). |
| BAA- 433 | 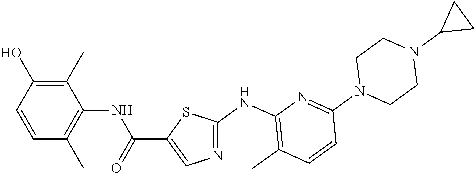 [SMILES: Cc1ccc(N2CCN(C3CC3)CC2)nc1Nc1ncc(C(=O)Nc2c(C)ccc(O)c2C)s1] 2-((6-(4-cyclopropylpiperazin-1-yl)-3- methylpyridin-2-yl)amino)-N-(3- hydroxy-2,6-dimethylphenyl)thiazole- 5-carboxamide | MS (ES+) m/z 479 (M + H). ¹H NMR (300 MHz, MeOD) δ 8.15 (s, 1H), 7.37 (dd, J = 8.3, 0.8 Hz, 1H), 6.97-6.88 (m, 1H), 6.69 (d, J = 8.2 Hz, 1H), 6.36 (d, J = 8.3 Hz, 1H), 3.61 (t, J = 5.1 Hz, 4H), 2.78 (t, J = 5.2 Hz, 4H), 2.26-2.23 (m, 3H), 2.18 (s, 3H), 2.13 (s, 3H), 1.71 (dq, J = 6.6, 3.5 Hz, 1H), 0.58- 0.41 (m, 4H). |
| BAA- 276 | 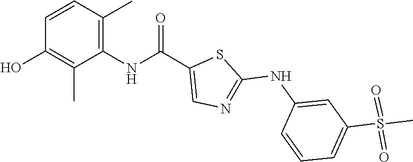 [SMILES: Cc1ccc(O)c(C)c1NC(=O)c1cnc(Nc2cccc(S(C)(=O)=O)c2)s1] N-(3-hydroxy-2,6-dimethylphenyl)-2- ((3- (methylsulfonyl)phenyl)amino)thiazole- 5-carboxamide | ¹H NMR (300 MHz, Methanol-d4/chloroform- d) δ 8.07 (s, 1H), 7.88-7.78 (m, 4H), 6.89 (d, J = 8.2 Hz, 1H), 6.68 (d, J = 8.2 Hz, 1H), 3.07 (s, 3H), 2.15 (s, 3H), 2.12 (s, 3H). MS (ES+) m/z 418.3 (M + H)⁺ |
| BAA- 277 | 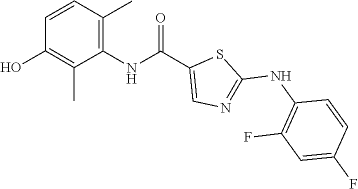 [SMILES: Cc1ccc(O)c(C)c1NC(=O)c1cnc(Nc2ccc(F)cc2F)s1] 2-((2,4-difluorophenyl)amino)-N-(3- hydroxy-2,6-dimethylphenyl)thiazole- 5-carboxamide | ¹H NMR (300 MHz, Methanol-d4/chloroform- d) δ 8.09 (s, 1H), 7.96 (s, 1H), 7.07-6.87 (m, 3H), 6.68 (d, J = 8.2 Hz, 1H), 2.15 (s, 3H), 2.10 (s, 3H). ¹⁹F NMR (282 MHz, Methanol-d4/chloroform-d) δ −116.91, −123.64. MS (ES+) m/z 376.3 (M + H)⁺ |
| BAA- 278 | 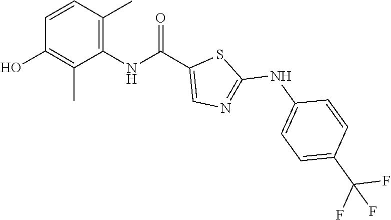 [SMILES: Cc1ccc(O)c(C)c1NC(=O)c1cnc(Nc2ccc(C(F)(F)F)cc2)s1] N-(3-hydroxy-2,6-dimethylphenyl)-2- ((4- (trifluoromethyl)phenyl)amino)thiazole- 5-carboxamide | ¹H NMR (300 MHz, Methanol-d4/chloroform- d) δ 8.07 (s, 1H), 7.82 (d, J = 8.5 Hz, 2H), 7.61 (d, J = 8.5 Hz, 2H), 6.91 (d, J = 8.5 Hz, 1H), 6.69 (d, J = 8.2 Hz, 1H), 2.16 (s, 3H), 2.12 (s, 3H). ¹⁹F NMR (282 MHz, Methanol- d4/chloroform-d) δ −63.24. MS (ES+) m/z 408.2 (M + H)⁺ |
| BAA- 279 | 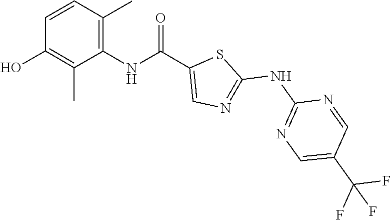 [SMILES: Cc1ccc(O)c(C)c1NC(=O)c1cnc(Nc2ncc(C(F)(F)F)cn2)s1] N-(3-hydroxy-2,6-dimethylphenyl)-2- ((5-(trifluoromethyl)pyrimidin-2- yl)amino)thiazole-5-carboxamide | ¹H NMR (300 MHz, Methanol-d4/chloroform- d) δ 8.85 (s, 2H), 8.21 (s, 1H), 6.90 (d, J = 8.2 Hz, 1H), 6.69 (d, J = 8.2 Hz, 1H), 2.16 (s, 3H), 2.13 (s, 3H). ¹⁹F NMR (282 MHz, Methanol-d4/chloroform-d) δ −62.18. MS (ES+) m/z 410.3 (M + H)⁺ |
| BAA- 280 | 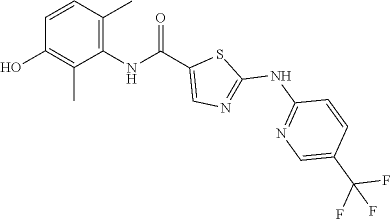 [SMILES: Cc1ccc(O)c(C)c1NC(=O)c1cnc(Nc2ccc(C(F)(F)F)cn2)s1] N-(3-hydroxy-2,6-dimethylphenyl)-2- ((5-(trifluoromethyl)pyridin-2- yl)amino)thiazole-5-carboxamide | ¹H NMR (300 MHz, Methanol-d4/chloroform- d) δ 8.62 (s, 1H), 8.15 (s, 1H), 7.88 (dd, J = 8.9, 2.4 Hz, 1H), 7.13 (d, J = 8.7 Hz, 1H), 6.90 (d, J = 8.2 Hz, 1H), 6.68 (d, J = 8.2 Hz, 1H), 2.16 (s, 3H), 2.12 (s, 3H). ¹⁹F NMR (282 MHz, Methanol-d4/chloroform-d) δ −62.50. MS (ES+) m/z 409.3 (M + H)⁺ |
| BAA- 281 | 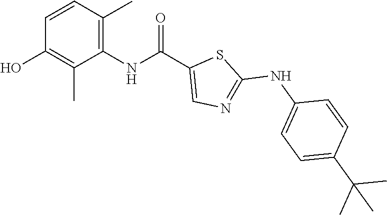 [SMILES: Cc1ccc(O)c(C)c1NC(=O)c1cnc(Nc2ccc(C(C)(C)C)cc2)s1] 2-((4-(tert-butyl)phenyl)amino)-N-(3- hydroxy-2,6-dimethylphenyl)thiazole- 5-carboxamide | ¹H NMR (300 MHz, Methanol-d4/chloroform- d) δ 7.96 (s, 1H), 7.44-7.34 (m, 4H), 6.89 (d, J = 8.2 Hz, 1H), 6.67 (d, J = 8.2 Hz, 1H), 2.15 (s, 3H), 2.11 (s, 3H), 1.31 (s, 9H). MS (ES+) m/z 396.3 (M + H)⁺ |
| BAA- 282 | 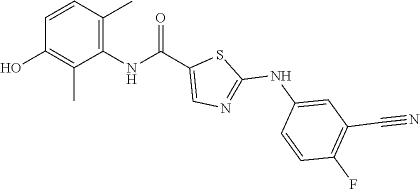 [SMILES: Cc1ccc(O)c(C)c1NC(=O)c1cnc(Nc2ccc(F)c(C#N)c2)s1] 2-((3-cyano-4-fluorophenyl)amino)-N- (3-hydroxy-2,6- dimethylphenyl)thiazole-5- carboxamide | ¹H NMR (300 MHz, Methanol-d4/chloroform- d) δ 8.20 (dd, J = 5.5, 2.8 Hz, 1H), 8.03 (s, 1H), 7.76 (ddd, J = 9.2, 4.5, 2.8 Hz, 1H), 7.24 (t, J = 8.9 Hz, 1H), 6.89 (d, J = 8.2 Hz, 1H), 6.68 (d, J = 8.2 Hz, 1H), 2.15 (s, 3H), 2.11 (s, 3H). MS (ES+) m/z 383.2 (M + H)⁺ |
| BAA- 283 | 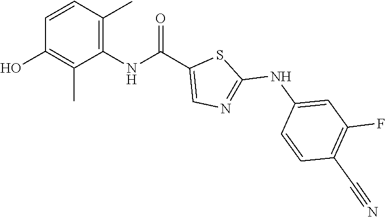 [SMILES: Cc1ccc(O)c(C)c1NC(=O)c1cnc(Nc2ccc(C#N)c(F)c2)s1] 2-((4-cyano-3-fluorophenyl)amino)-N- (3-hydroxy-2,6- dimethylphenyl)thiazole-5- carboxamide | ¹H NMR (300 MHz, Methanol-d4/chloroform- d) δ 8.08 (s, 1H), 7.93 (dd, J = 12.2, 2.1 Hz, 1H), 7.53 (dd, J = 8.7, 7.3 Hz, 1H), 7.29 (dd, J = 8.6, 2.1 Hz, 1H), 6.89 (d, J = 8.0 Hz, 1H), 6.67 (d, J = 8.2 Hz, 1H), 2.15 (s, 3H), 2.12 (s, 3H). MS (ES+) m/z 383.2 (M + H)⁺ |
| BAA- 284 | 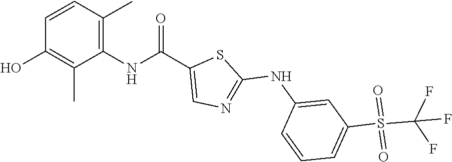 [SMILES: Cc1ccc(O)c(C)c1NC(=O)c1cnc(Nc2cccc(S(=O)(=O)C(F)(F)F)c2)s1] N-(3-hydroxy-2,6-dimethylphenyl)-2- ((3- ((trifluoromethyl)sulfonyl)phenyl)amino) thiazole-5-carboxamide | 1H NMR (300 MHz, Methanol-d4/chloroform- d) δ 8.61 (br, 1H), 8.07 (s, 1H), 8.03-7.94 (m, 1H), 7.68-7.59 (m, 2H), 6.89 (d, J = 8.0 Hz, 1H), 6.68 (d, J = 8.2 Hz, 1H), 2.16 (s, 3H), 2.12 (s, 3H). MS (ES+) m/z 472.2 (M + H)⁺ |
| BAA- 285 | 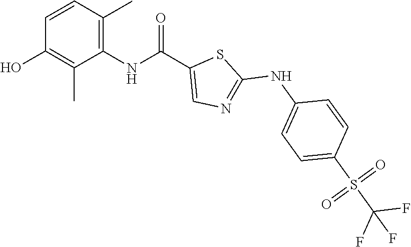 [SMILES: Cc1ccc(O)c(C)c1NC(=O)c1cnc(Nc2ccc(S(=O)(=O)C(F)(F)F)cc2)s1] N-(3-hydroxy-2,6-dimethylphenyl)-2- ((4. ((trifluoromethyl)sulfonyl)phenyl)amino) thiazole-5-carboxamide | ¹H NMR (300 MHz, Methanol-d4/chloroform- d) δ 8.13 (s, 1H), 8.08-7.95 (m, 4H), 6.92 (d, J = 8.2 Hz, 1H), 6.69 (d, J = 8.2 Hz, 1H), 2.16 (s, 3H), 2.12 (s, 3H). ¹⁹F NMR (282 MHz, Methanol-d4/chloroform-d) δ −80.83. MS (ES+) m/z 472.2 (M + H)⁺ |
| BAA- 286 | 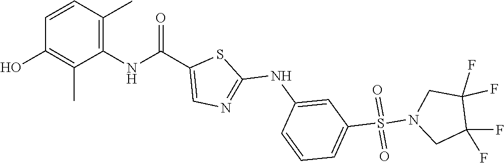 [SMILES: Cc1ccc(O)c(C)c1NC(=O)c1cnc(Nc2cccc(S(=O)(=O)N3CC(F)(F)C(F)(F)C3)c2)s1] N-(3-hydroxy-2,6-dimethylphenyl)-2- ((3-((3,3,4,4-tetrafluoropyrrolidin-1- yl)sulfonyl)phenyl)amino)thiazole-5- carboxamide | ¹H NMR (300 MHz, Methanol-d4/chloroform- d) δ 8.35 (s, 1H), 8.04 (s, 1H), 7.78 (d, J = 7.9 Hz, 1H), 7.55 (t, J = 8.0 Hz, 1H), 7.44 (d, J = 8.5 Hz, 1H), 6.89 (d, J = 8.2 Hz, 1H), 6.68 (d, J = 8.2 Hz, 1H), 3.88-3.70 (m, 4H), 2.15 (s, 3H), 2.12 (s, 3H). ¹⁹F NMR (282 MHz, Methanol-d4/chloroform-d) δ −123.01- −123.22 (m). MS (ES+) m/z 545.2 (M + H)⁺ |
| BAA- 259 | 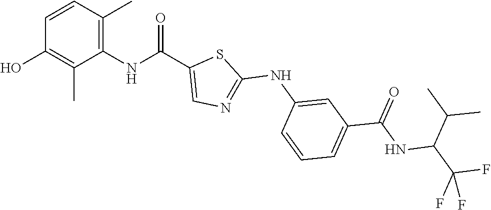 [SMILES: Cc1ccc(O)c(C)c1NC(=O)c1cnc(Nc2cccc(C(=O)NC(C(C)C)C(F)(F)F)c2)s1] N-(3-hydroxy-2,6-dimethylphenyl)-2- ((3-((1, 1, 1-trifluoro-3-methylbutan-2- yl)carbamoyl)phenyl)amino)thiazole-5- carboxamide | ¹H NMR (300 MHz, Methanol-d4/chloroform- d) δ 8.08 (t, J = 1.9 Hz, 1H), 8.04 (s, 1H), 7.66 (ddd, J = 7.8, 2.4, 1.3 Hz, 1H), 7.53- 7.29 (m, 2H), 6.89 (d, J = 8.2 Hz, 1H), 6.68 (d, J = 8.2 Hz, 1H), 4.70-4.55 (m, 1H), 2.23 (h, J = 6.8 Hz, 1H), 2.16 (s, 3H), 2.12 (s, 3H), 1.08 (app. t, J = 7.3 Hz, 6H). ¹⁹F NMR (proton decoupled, 282 MHz, MeOD) δ −72.32. MS (ES+) m/z 507.3 (M + H)⁺ |
| BAA- 261 | 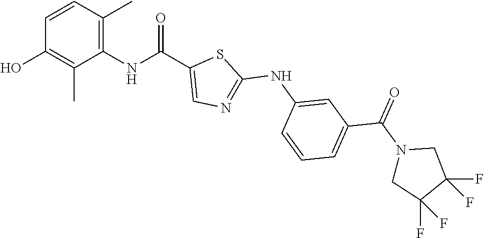 [SMILES: Cc1ccc(O)c(C)c1NC(=O)c1cnc(Nc2cccc(C(=O)N3CC(F)(F)C(F)(F)C3)c2)s1] N-(3-hydroxy-2,6-dimethylphenyl)-2- ((3-(3,3,4,4-tetrafluoropyrrolidine-1- carbonyl)phenyl)amino)thiazole-5- carboxamide | H NMR (300 MHz, MeOD) δ 8.03 (s, 1H), 7.99 (br, 1H), 7.65 (d, J = 8.3 Hz, 1H), 7.45 (t, J = 7.9 Hz, 1H), 7.21 (d, J = 7.7 Hz, 1H), 6.90 (d, J = 8.2 Hz, 1H), 6.68 (d, J = 8.2 Hz, 1H), 4.18 (t, J = 13.4 Hz, 4H), 2.16 (s, 3H), 2.11 (s, 3H).¹⁹F NMR (proton decoupled, 282 MHz, MeOD) δ −124.16-−124.82 (m). MS (ES+) m/z 509.3 (M + H)⁺ |
| BAA- 287 | 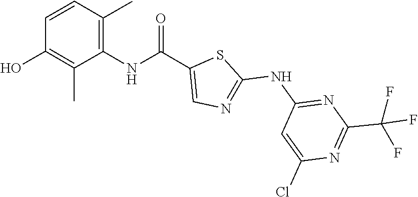 [SMILES: Cc1ccc(O)c(C)c1NC(=O)c1cnc(Nc2cc(Cl)nc(C(F)(F)F)n2)s1] 2-((6-chloro-2- (trifluoromethyl)pyrimidin-4-yl)amino)- N-(3-hydroxy-2,6- dimethylphenyl)thiazole-5- carboxamide | ¹H NMR (300 MHz, Methanol-d4/chloroform- d) δ 8.23 (s, 1H), 7.28 (s, 1H), 6.91 (d, J = 8.2 Hz, 1H), 6.69 (d, J = 8.2 Hz, 1H), 2.16 (d, J = 4.1 Hz, 3H), 2.12 (s, 3H). ¹⁹F NMR (proton decoupled, 282 MHz, Methanol- d4/chloroform-d) δ −72.30. MS (ES+) m/z 444.4 (M + H)⁺ |
| BAA- 288 | 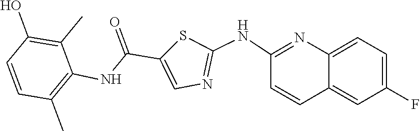 [SMILES: Cc1ccc(O)c(C)c1NC(=O)c1cnc(Nc2ccc3cc(F)ccc3n2)s1] 2-((6-fluoroquinolin-2-yl)amino)-N-(3- hydroxy-2,6-dimethylphenyl)thiazole- 5-carboxamide | ¹H NMR (300 MHz, Methanol-d4/chloroform- d) δ 8.19 (s, 1H), 8.11 (d, J = 9.0 Hz, 1H), 8.02 (m, 1H), 7.56-7.40 (m, 2H), 7.23 (m, 1H), 6.90 (d, J = 8.3 Hz, 1H), 6.69 (d, J = 8.2 Hz, 1H), 2.17 (s, 3H), 2.14 (s, 3H). MS (ES+) m/z 409.3 (M + H)⁺ |
| BAA- 289 | 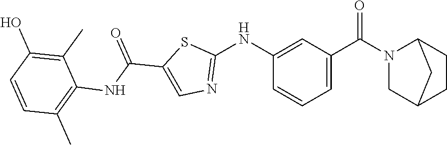 [SMILES: Cc1ccc(O)c(C)c1NC(=O)c1cnc(Nc2cccc(C(=O)N3CC4CCC3C4)c2)s1] 2-((3-(2-azabicyclo[2.2.1]heptane-2- carbonyl)phenyl)amino)-N-(3-hydroxy- 2,6-dimethylphenyl)thiazole-5- carboxamide | ¹H NMR (300 MHz, Methanol-d4/chloroform- d) for a mixture of isomers δ 8.02 (s, 1H), 7.93 (br, 0.7H), 7.82 (br, 0.3H), 7.58 (m, 1H), 7.46-7.34 (m, 1H), 7.18-7.08 (m, 1H), 6.96-6.87 (m, 1H), 6.77-6.64 (m, 1H), 4.66 (br, 0.3H), 4.24 (br, 0.7H), 3.54 (m, 1H), 3.18 (d, J = 11.2 Hz, 0.7H), 3.09 (d, J = 9.6 Hz, 0.3H), 2.69 (br, 0.7H), 2.59 (br, 0.3H), 2.20-2.09 (m, 6H), 1.96-1.44 (m, 6H). MS (ES+) m/z 463.3 (M + H)⁺ |
| BAA- 290 | 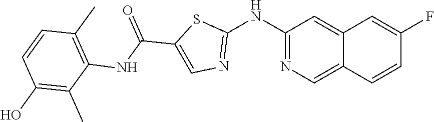 [SMILES: Cc1ccc(O)c(C)c1NC(=O)c1cnc(Nc2cc3cc(F)ccc3cn2)s1] 2-((6-fluoroisoquinolin-3-yl)amino)-N- (3-hydroxy-2,6- dimethylphenyl)thiazole-5- carboxamide | ¹H NMR (300 MHz, Methanol-d4/chloroform- d) δ 9.12 (s, 1H), 8.13 (s, 1H), 8.04 (dd, J = 9.1, 5.6 Hz, 1H), 7.47-7.34 (m, 2H), 7.23 (td, J = 8.8, 2.4 Hz, 1H), 6.91 (d, J = 8.2 Hz, 1H), 6.69 (d, J = 8.2 Hz, 1H), 2.18 (s, 3H), 2.14 (s, 3H). ¹⁹F NMR (282 MHz, Methanol- d4/chloroform-d) δ −108.36. MS (ES+) m/z 409.7 (M + H)⁺ |
| BAA- 291 | 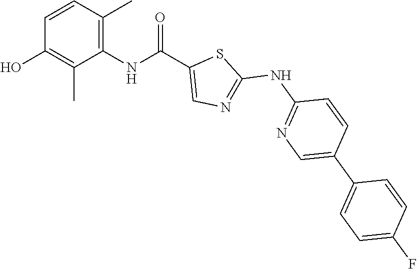 [SMILES: Cc1ccc(O)c(C)c1NC(=O)c1cnc(Nc2ccc(-c3ccc(F)cc3)cn2)s1] 2-((5-(4-fluorophenyl)pyridin-2- yl)amino)-N-(3-hydroxy-2,6- dimethylphenyl)thiazole-5- carboxamide | ¹H NMR (300 MHz, Methanol-d4/chloroform- d) δ 8.55 (dd, J = 2.4, 0.8 Hz, 1H), 8.12 (s, 1H), 7.87 (dd, J = 8.6, 2.4 Hz, 1H), 7.58- 7.50 (m, 2H), 7.24-7.06 (m, 3H), 6.90 (d, J = 8.3 Hz, 1H), 6.68 (d, J = 8.2 Hz, 1H), 2.17 (s, 3H), 2.13 (s, 3H). ¹⁹F NMR (282 MHz, Methanol-d4/chloroform-d) δ −116.14- −116.30 (m). MS (ES+) m/z 435.7 (M + H)⁺ |
| BAA- 292 | 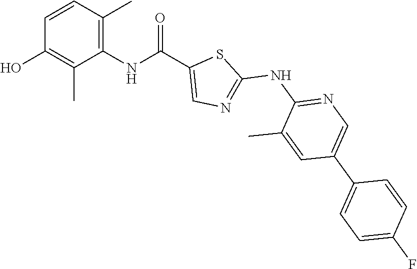 [SMILES: Cc1cc(-c2ccc(F)cc2)cnc1Nc1ncc(C(=O)Nc2c(C)ccc(O)c2C)s1] 2-((5-(4-fluorophenyl)-3-methylpyridin- 2-yl)amino)-N-(3-hydroxy-2,6- dimethylphenyl)thiazole-5- carboxamide | ¹H NMR (300 MHz, Methanol-d4/chloroform- d) δ 8.45 (d, J = 2.3 Hz, 1H), 8.16 (s, 1H), 7.76 (dd, J = 2.3, 1.0 Hz, 1H), 7.62-7.53 (m, 2H), 7.22-7.09 (m, 2H), 6.91 (d, J = 8.2 Hz, 1H), 6.69 (d, J = 8.2 Hz, 1H), 2.45 (s, 3H), 2.18 (s, 3H), 2.13 (s, 3H). ¹⁹F NMR (282 MHz, Methanol-d4/chloroform-d) δ −116.66- −116.80 (m). MS (ES+) m/z 449.6 (M + H)⁺ |
| BAA- 293 | 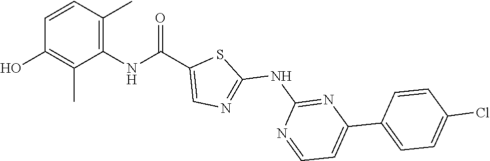 [SMILES: Cc1ccc(O)c(C)c1NC(=O)c1cnc(Nc2nccc(-c3ccc(Cl)cc3)n2)s1] 2-((4-(4-chlorophenyl)pyrimidin-2- yl)amino)-N-(3-hydroxy-2,6- dimethylphenyl)thiazole-5- carboxamide | ¹H NMR (300 MHz, Methanol-d4/chloroform- d) δ 8.67 (d, J = 5.3 Hz, 1H), 8.31-8.24 (m, 2H), 8.21 (s, 1H), 7.58-7.50 (m, 3H), 6.92 (d, J = 8.3 Hz, 1H), 6.70 (d, J = 8.2 Hz, 1H), 2.19 (s, 3H), 2.14 (s, 3H). MS (ES+) m/z 452.2/454.2 (M + H)⁺ |
| BAA- 294 | 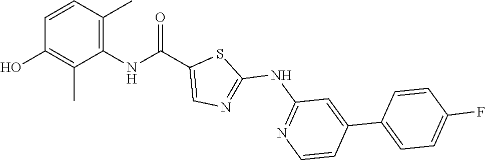 [SMILES: Cc1ccc(O)c(C)c1NC(=O)c1cnc(Nc2cc(-c3ccc(F)cc3)ccn2)s1] 2-((4-(4-fluorophenyl)pyridin-2- yl)amino)-N-(3-hydroxy-2,6- dimethylphenyl)thiazole-5- carboxamide | ¹H NMR (300 MHz, Methanol-d4/chloroform- d) δ 8.38 (d, J = 5.4 Hz, 1H), 8.13 (s, 1H), 7.76-7.63 (m, 2H), 7.27-7.14 (m, 4H), 6.90 (d, J = 8.2 Hz, 1H), 6.69 (d, J = 8.2 Hz, 1H), 2.17 (s, 3H), 2.13 (s, 3H). ¹⁹F NMR (282 MHz, Methanol-d4/chloroform-d) δ −113.88. MS (ES+) m/z 435.7 (M + H)⁺ |
| BAA- 295 | 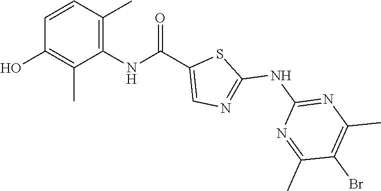 [SMILES: Cc1ccc(O)c(C)c1NC(=O)c1cnc(Nc2nc(C)c(Br)c(C)n2)s1] 2-((5-bromo-4,6-dimethylpyrimidin-2- yl)amino)-N-(3-hydroxy-2,6- dimethylphenyl)thiazole-5- carboxamide | ¹H NMR (300 MHz, Methanol-d4/chloroform- d) δ 8.15 (s, 1H), 6.92 (d, J = 8.1 Hz, 1H), 6.71 (d, J = 8.2 Hz, 1H), 2.63 (s, 6H), 2.18 (s, 3H), 2.15 (s, 3H). MS (ES+) m/z 448.5/450.5 (M + H)⁺ |
| BAA- 296 | 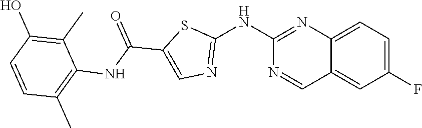 [SMILES: Cc1ccc(O)c(C)c1NC(=O)c1cnc(Nc2ncc3cc(F)ccc3n2)s1] 2-((6-fluoroquinazolin-2-yl)amino)-N- (3-hydroxy-2,6- dimethylphenyl)thiazole-5- carboxamide | ¹H NMR (300 MHz, DMSO) δ 12.32 (s, 1H), 9.65 (s, 1H), 9.49 (s, 1H), 9.19 (s, 1H), 8.30 (s, 1H), 7.97-7.79 (m, 3H), 6.90 (d, J = 8.2 Hz, 1H), 6.70 (d, J = 8.2 Hz, 1H), 2.10 (s, 3H), 2.02 (s, 3H). ¹⁹F NMR (282 MHz, DMSO) δ −114.89 (td, J = 8.4, 5.1 Hz). MS (ES+) m/z 410.5 (M + H)⁺ |
| BAA- 297 | 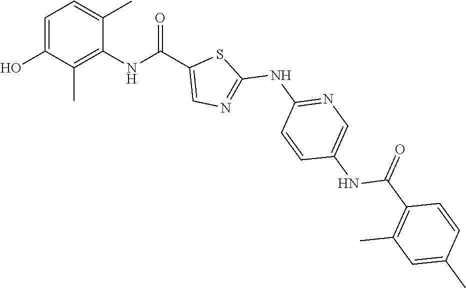 [SMILES: Cc1ccc(C(=O)Nc2ccc(Nc3ncc(C(=O)Nc4c(C)ccc(O)c4C)s3)nc2)c(C)c1] 2-((5-(2,4-dimethylbenzamido)pyridin- 2-yl)amino)-N-(3-hydroxy-2,6- dimethylphenyl)thiazole-5- carboxamide | ¹H NMR (300 MHz, Methanol-d4/chloroform- d) δ 8.54 (d, J = 2.5 Hz, 1H), 8.17-8.06 (m, 2H), 7.37 (d, J = 7.6 Hz, 1H), 7.11-6.98 (m, 3H), 6.89 (d, J = 8.3 Hz, 1H), 6.68 (d, J = 8.2 Hz, 1H), 2.42 (s, 3H), 2.32 (s, 3H), 2.16 (s, 3H), 2.12 (s, 3H). MS (ES+) m/z 488.5 (M + H)⁺ |
| BAA- 298 | 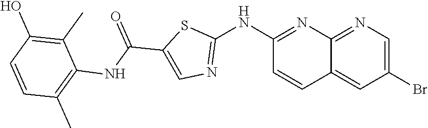 [SMILES: Cc1ccc(O)c(C)c1NC(=O)c1cnc(Nc2ccc3cc(Br)cnc3n2)s1] 2-((6-bromo-1,8-naphthyridin-2- yl)amino)-N-(3-hydroxy-2,6- dimethylphenyl)thiazole-5- carboxamide | ¹H NMR (300 MHz, DMSO) δ 9.54 (s, 1H), 9.38-9.01 (br, 1H), 8.86 (s, 1H), 8.53 (s, 1H), 8.27 (s, 1H), 8.16 (m, 1H), 7.29 (m, 1H), 7.21-6.79 (br, 1H), 6.89 (d, J = 8.3 Hz, 1H), 6.69 (d, J = 8.2 Hz, 1H), 2.09 (s, 3H), 2.01 (s, 3H). MS (ES+) m/z 470.4/472.4 (M + H)⁺ |
| BAA- 299 | 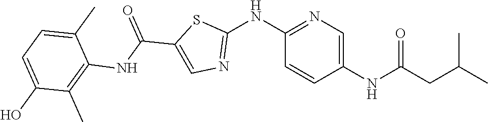 [SMILES: Cc1ccc(O)c(C)c1NC(=O)c1cnc(Nc2ccc(NC(=O)CC(C)C)cn2)s1] N-(3-hydroxy-2,6-dimethylphenyl)-2- ((5-(3-methylbutanamido)pyridin-2- yl)amino)thiazole-5-carboxamide | ¹H NMR (300 MHz, Methanol-d4/chloroform- d) δ 8.48 (dd, J = 2.6, 0.8 Hz, 1H), 8.13 (s, 1H), 7.99 (dd, J = 8.9, 2.6 Hz, 1H), 7.01 (dd, J = 8.9, 0.8 Hz, 1H), 6.90 (d, J = 8.2 Hz, 1H), 6.68 (d, J = 8.2 Hz, 1H), 2.27-2.17 (m, 3H), 2.17 (s, 3H), 2.13 (s, 3H), 1.01 (s, 3H), 0.99 (s, 3H). MS (ES+) m/z 440.5 (M + H)⁺ |
| BAA- 300 | 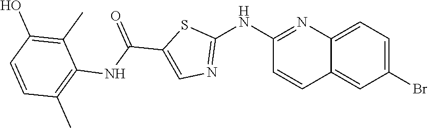 [SMILES: Cc1ccc(O)c(C)c1NC(=O)c1cnc(Nc2ccc3cc(Br)ccc3n2)s1] 2-((6-bromoquinolin-2-yl)amino)-N-(3- hydroxy-2,6-dimethylphenyl)thiazole- 5-carboxamide | ¹H NMR (300 MHz, DMSO) δ 9.63 (s, 1H), 9.25 (s, 1H), 8.29 (s, 1H), 8.26 (d, J = 9.0 Hz, 1H), 8.15 (d, J = 1.9 Hz, 1H), 7.88-7.79 (m, 2H), 7.44-7.29 (m, 2H), 6.91 (d, J = 8.2 Hz, 1H), 6.71 (d, J = 8.1 Hz, 1H), 2.11 (s, 3H), 2.02 (s, 3H). MS (ES+) m/z 469.4/471.4 (M + H)⁺ |
| BAA- 301 | 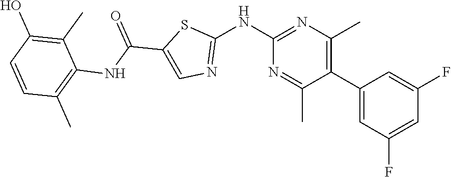 [SMILES: Cc1ccc(O)c(C)c1NC(=O)c1cnc(Nc2nc(C)c(-c3cc(F)cc(F)c3)c(C)n2)s1] 2-((5-(3,5-difluorophenyl)-4,6- dimethylpyrimidin-2-yl)amino)-N-(3- hydroxy-2,6-dimethylphenyl)thiazole- 5-carboxamide | ¹H NMR (300 MHz, Methanol-d4/chloroform- d) δ 8.11 (s, 1H), 6.94-6.84 (m, 2H), 6.78- 6.65 (m, 3H), 2.26 (s, 6H), 2.15 (s, 3H), 2.12 (s, 3H). ¹⁹F NMR (282 MHz, Methanol- d4/chloroform-d) δ −108.82-−109.00 (m). MS (ES+) m/z 482.3 (M + H)⁺ |
| BAA- 302 | 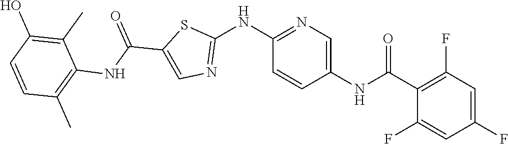 [SMILES: Cc1ccc(O)c(C)c1NC(=O)c1cnc(Nc2ccc(NC(=O)c3c(F)cc(F)cc3F)cn2)s1] N-(3-hydroxy-2,6-dimethylphenyl)-2- ((5-(2,4,6-trifluorobenzamido)pyridin- 2-yl)amino)thiazole-5-carboxamide | ¹H NMR (300 MHz, DMSO) δ 11.64 (br, 1H), 10.95 (s, 1H), 9.56 (s, 1H), 9.20 (br, 1H), 8.64 (d, J = 2.6 Hz, 1H), 8.22 (s, 1H), 8.09 (dd, J = 8.9, 2.6 Hz, 1H), 7.49-7.36 (m, 2H), 7.17 (d, J = 8.9 Hz, 1H), 6.90 (d, J = 8.2 Hz, 1H), 6.70 (d, J = 8.2 Hz, 1H), 2.09 (s, 3H), 2.01 (s, 3H). ¹⁹F NMR (282 MHz, DMSO) δ −104.90 (tt, J = 9.4, 7.4 Hz), −110.50-−110.62 (m, 2F). MS (ES+) m/z 514.3 (M + H)⁺ |
| BAA- 303 | 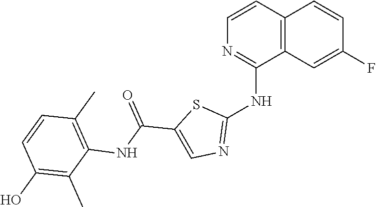 [SMILES: Cc1ccc(O)c(C)c1NC(=O)c1cnc(Nc2nccc3ccc(F)cc23)s1] 2-((7-fluoroisoquinolin-1-yl)amino)-N- (3-hydroxy-2,6- dimethylphenyl)thiazole-5- carboxamide | ¹H NMR (300 MHz, Methanol-d4/chloroform- d) δ 8.38-8.02 (m, 3H), 7.88 (br, 1H), 7.52 (br, 1H), 7.33 (br, 1H), 6.92 (d, J = 8.2 Hz, 1H), 6.71 (d, J = 8.2 Hz, 1H), 2.19 (s, 3H), 2.16 (s, 3H). ¹⁹F NMR (282 MHz, Methanol- d4/chloroform-d) δ −111.29-−112.36 (m). MS (ES+) m/z 409.3 (M + H)⁺ |
| BAA- 304 | 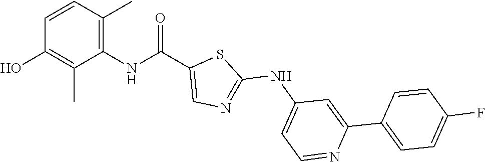 [SMILES: Cc1ccc(O)c(C)c1NC(=O)c1cnc(Nc2ccnc(-c3ccc(F)cc3)c2)s1] 2-((2-(4-fluorophenyl)pyridin-4- yl)amino)-N-(3-hydroxy-2,6- dimethylphenyl)thiazole-5- carboxamide | ¹H NMR (300 MHz, Methanol-d4/chloroform- d) δ 8.39 (d, J = 5.8 Hz, 1H), 8.10 (s, 1H), 8.04 (d, J = 2.1 Hz, 1H), 7.92-7.82 (m, 2H), 7.51 (dd, J = 5.8, 2.2 Hz, 1H), 7.23-7.12 (m, 2H), 6.90 (d, J = 8.3 Hz, 1H), 6.69 (d, J = 8.2 Hz, 1H), 2.16 (s, 3H), 2.12 (s, 3H). ¹⁹F NMR (282 MHz, Methanol-d4/chloroform-d) δ −113.78 (tt, J = 8.7, 5.4 Hz). MS (ES+) m/z 435.3 (M + H)⁺ |
| BAA- 305 | 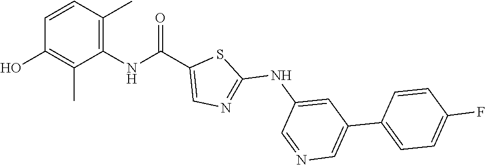 [SMILES: Cc1ccc(O)c(C)c1NC(=O)c1cnc(Nc2cncc(-c3ccc(F)cc3)c2)s1] 2-((5-(4-fluorophenyl)pyridin-3- yl)amino)-N-(3-hydroxy-2,6- dimethylphenyl)thiazole-5- carboxamide | ¹H NMR (300 MHz, Methanol-d4/chloroform- d) δ 8.62 (d, J = 2.5 Hz, 1H), 8.44 (t, J = 2.2 Hz, 1H), 8.35 (d, J = 2.0 Hz, 1H), 8.01 (s, 1H), 7.65-7.55 (m, 2H), 7.22-7.13 (m, 2H), 6.88 (d, J = 8.2 Hz, 1H), 6.68 (d, J = 8.2 Hz, 1H), 2.15 (s, 3H), 2.11 (s, 3H). ¹⁹F NMR (282 MHz, Methanol-d4/chloroform-d) δ −114.44 (tt, J = 8.6, 5.3 Hz). MS (ES+) m/z 435.3 (M + H)⁺ |
| BAA- 306 | 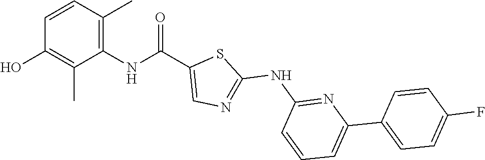 [SMILES: Cc1ccc(O)c(C)c1NC(=O)c1cnc(Nc2cccc(-c3ccc(F)cc3)n2)s1] 2-((6-(4-fluorophenyl)pyridin-2- yl)amino)-N-(3-hydroxy-2,6- dimethylphenyl)thiazole-5- carboxamide | ¹H NMR (300 MHz, Methanol-d4/chloroform- d) δ 8.27-8.09 (m, 3H), 7.80-7.70 (m, 1H), 7.40 (d, J = 7.6 Hz, 1H), 7.23-7.13 (m, 2H), 6.97 (d, J = 8.2 Hz, 1H), 6.90 (d, J = 8.2 Hz, 1H), 6.69 (d, J = 8.2 Hz, 1H), 2.18 (s, 3H), 2.14 (s, 3H). ¹⁹F NMR (282 MHz, Methanol- d4/chloroform-d) δ −114.09 (tt, J = 8.7, 5.4 Hz). MS (ES+) m/z 435.5 (M + H)⁺ |
| BAA- 307 | 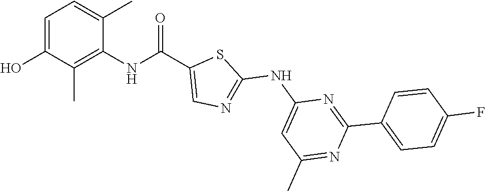 [SMILES: Cc1cc(Nc2ncc(C(=O)Nc3c(C)ccc(O)c3C)s2)nc(-c2ccc(F)cc2)n1] 2-((2-(4-fluorophenyl)-6- methylpyrimidin-4-yl)amino)-N-(3- hydroxy-2,6-dimethylphenyl)thiazole- 5-carboxamide | ¹H NMR (300 MHz, DMSO) δ 12.10 (s, 1H), 9.70 (s, 1H), 9.19 (s, 1H), 8.60-8.50 (m, 2H), 8.31 (s, 1H), 7.36 (t, J = 8.9 Hz, 2H), 6.91 (d, J = 8.2 Hz, 1H), 6.87 (s, 1H), 6.70 (d, J = 8.2 Hz, 1H), 2.48 (s, 3H), 2.10 (s, 3H), 2.02 (s, 3H). ¹⁹F NMR (282 MHz, DMSO) δ −110.12 (tt, J = 8.6, 5.5 Hz). MS (ES+) m/z 450.3 (M + H)⁺ |
| BAA- 308 | 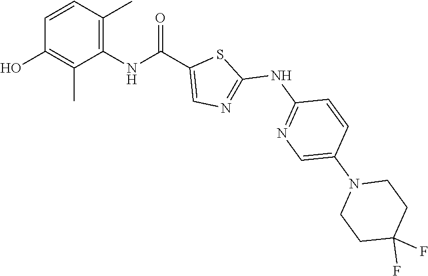 [SMILES: Cc1ccc(O)c(C)c1NC(=O)c1cnc(Nc2ccc(N3CCC(F)(F)CC3)cn2)s1] 2-((5-(4,4-difluoropiperidin-1- yl)pyridin-2-yl)amino)-N-(3-hydroxy- 2,6-dimethylphenyl)thiazole-5- carboxamide | ¹H NMR (300 MHz, Methanol-d4/chloroform- d) δ 8.11 (s, 1H), 8.06 (d, J = 2.8 Hz, 1H), 7.44 (dd, J = 9.0, 2.9 Hz, 1H), 6.98 (d, J = 9.0 Hz, 1H), 6.89 (d, J = 8.3 Hz, 1H), 6.68 (d, J = 8.2 Hz, 1H), 3.30-3.21 (m, 4H), 2.16 (s, 3H), 2.18-2.02 (m, 7H). ¹⁹F NMR (282 MHz, Methanol-d4/chloroform-d) δ −99.09 (p, J = 13.7 Hz). MS (ES+) m/z 460.3 (M + H)⁺ |
| BAA- 309 | 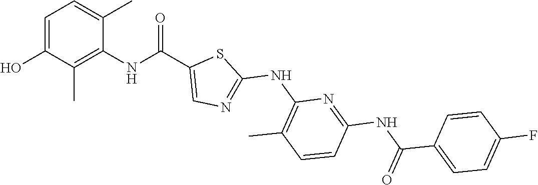 [SMILES: Cc1ccc(NC(=O)c2ccc(F)cc2)nc1Nc1ncc(C(=O)Nc2c(C)ccc(O)c2C)s1] 2-((6-(4-fluorobenzamido)-3- methylpyridin-2-yl)amino)-N-(3- hydroxy-2,6-dimethylphenyl)thiazole- 5-carboxamide | ¹H NMR (300 MHz, Methanol-d4/chloroform- d) δ 8.15 (s, 1H), 8.05-7.96 (m, 2H), 7.77 (d, J = 8.0 Hz, 1H), 7.58 (dd, J = 8.1, 0.9 Hz, 1H), 7.25-7.14 (m, 2H), 6.90 (d, J = 8.2 Hz, 1H), 6.69 (d, J = 8.2 Hz, 1H), 2.35 (s, 3H), 2.16 (s, 3H), 2.13 (s, 3H). ¹⁹F NMR (282 MHz, Methanol-d4/chloroform-d) δ −108.14 (tt, J = 8.5, 5.2 Hz). MS (ES+) m/z 492.3 (M + H)⁺ |
| BAA- 310 | 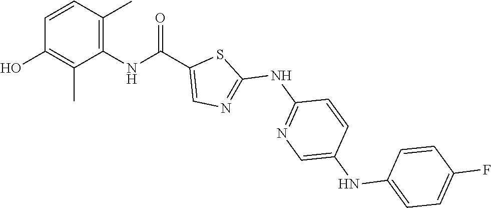 [SMILES: Cc1ccc(O)c(C)c1NC(=O)c1cnc(Nc2ccc(Nc3ccc(F)cc3)cn2)s1] 2-((5-((4-fluorophenyl)amino)pyridin-2- yl)amino)-N-(3-hydroxy-2,6- dimethylphenyl)thiazole-5- carboxamide | ¹H NMR (300 MHz, Methanol-d4/chloroform- d) δ 8.12 (d, J = 2.7 Hz, 1H), 8.08 (s, 1H), 7.44 (dd, J = 8.8, 2.8 Hz, 1H), 7.00-6.85 (m, 6H), 6.67 (d, J = 8.2 Hz, 1H), 2.15 (s, 3H), 2.12 (s, 3H). ¹⁹F NMR (282 MHz, Methanol- d4/chloroform-d) δ −124.60 (tt, J = 8.0, 5.0 Hz). MS (ES+) m/z 450.3 (M + H)⁺ |
| BAA- 311 | 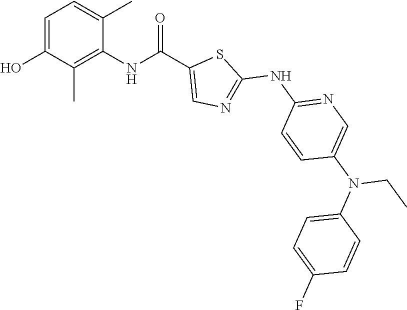 [SMILES: CCN(c1ccc(F)cc1)c1ccc(Nc2ncc(C(=O)Nc3c(C)ccc(O)c3C)s2)nc1] 2-((5-(ethyl(4- fluorophenyl)amino)pyridin-2- yl)amino)-N-(3-hydroxy-2,6- dimethylphenyl)thiazole-5- carboxamide | ¹H NMR (300 MHz, Methanol-d4/chloroform- d) δ 8.10 (s, 1H), 8.06 (dd, J = 2.8, 0.7 Hz, 1H), 7.33 (dd, J = 8.9, 2.8 Hz, 1H), 7.04- 6.90 (m, 6H), 6.74-6.64 (m, 1H), 3.73 (q, J = 7.1 Hz, 2H), 2.18 (s, 3H), 2.14 (s, 3H), 1.22 (t, J = 7.1 Hz, 3H). ¹⁹F NMR (282 MHz, Methanol-d4/chloroform-d) δ −123.05 (tt, J = 8.0, 5.0 Hz). MS (ES+) m/z 478.3 (M + H)⁺ |
| BAA- 312 | 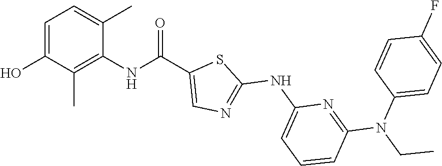 [SMILES: CCN(c1ccc(F)cc1)c1cccc(Nc2ncc(C(=O)Nc3c(C)ccc(O)c3C)s2)n1] 2-((6-(ethyl(4- fluorophenyl)amino)pyridin-2- yl)amino)-N-(3-hydroxy-2,6- dimethylphenyl)thiazole-5- carboxamide | ¹H NMR (300 MHz, DMSO) δ 11.36 (br, 1H), 9.52 (s, 1H), 9.18 (br, 1H), 8.20 (s, 1H), 7.42- 7.23 (m, 5H), 6.89 (d, J = 8.2 Hz, 1H), 6.69 (d, J = 8.2 Hz, 1H), 6.34 (d, J = 7.8 Hz, 1H), 5.75 (d, J = 9.1 Hz, 1H), 4.17-4.00 (m, 3H), 2.09 (s, 3H), 2.01 (s, 3H), 1.19 (t, J = 7.0 Hz, 2H). ¹⁹F NMR (282 MHz, DMSO) δ −115.55 (tt, J = 8.4, 5.3 Hz). MS (ES+) m/z 478.3 (M + H)⁺ |
| BAA- 313 | 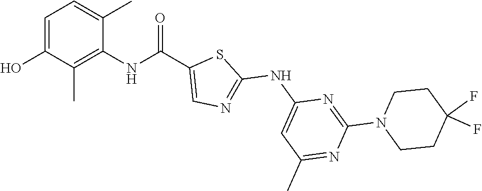 [SMILES: Cc1cc(Nc2ncc(C(=O)Nc3c(C)ccc(O)c3C)s2)nc(N2CCC(F)(F)CC2)n1] 2-((2-(4,4-difluoropiperidin-1-yl)-6- methylpyrimidin-4-yl)amino)-N-(3- hydroxy-2,6-dimethylphenyl)thiazole- 5-carboxamide | ¹H NMR (300 MHz, DMSO) δ 11.72 (s, 1H), 9.62 (s, 1H), 9.19 (s, 1H), 8.24 (s, 1H), 6.89 (d, J = 8.2 Hz, 1H), 6.69 (d, J = 8.2 Hz, 1H), 6.21 (s, 1H), 3.97 (t, J = 5.8 Hz, 4H), 2.23 (s, 3H), 2.07 (s, 3H), 2.00 (s, 3H), 2.12-1.92 (m, 4H). ¹⁹F NMR (282 MHz, DMSO) δ −94.73 (p, J = 14.2 Hz). MS (ES−) 473.4 m/z (M − H)⁻ |
| BAA- 314 | 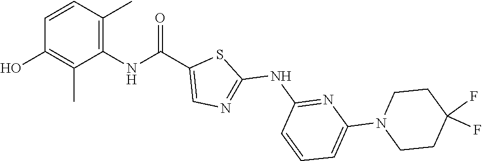 [SMILES: Cc1ccc(O)c(C)c1NC(=O)c1cnc(Nc2cccc(N3CCC(F)(F)CC3)n2)s1] 2-((6-(4,4-difluoropiperidin-1- yl)pyridin-2-yl)amino)-N-(3-hydroxy- 2, 6-dimethylphenyl)thiazole-5- carboxamide | ¹H NMR (300 MHz, Methanol-d4/chloroform- d) δ 8.11 (s, 1H), 7.48 (t, J = 8.0 Hz, 1H), 6.90 (d, J = 8.0 Hz, 1H), 6.68 (d, J = 8.2 Hz, 1H), 6.36 (dd, J = 15.6, 8.0 Hz, 2H), 3.90- 3.79 (m, 4H), 2.17 (s, 3H), 2.13 (s, 3H), 2.11- 1.92 (m, 4H). ¹⁹F NMR (282 MHz, Methanol-d4/chloroform-d) δ −97.36 (p, J = 13.9 Hz). MS (ES+) m/z 460.3 (M + H)⁺ |
| BAA- 315 | 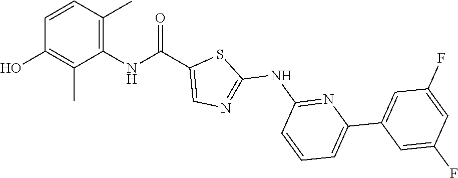 [SMILES: Cc1ccc(O)c(C)c1NC(=O)c1cnc(Nc2cccc(-c3cc(F)cc(F)c3)n2)s1] 2-((6-(3,5-difluorophenyl)pyridin-2- yl)amino)-N-(3-hydroxy-2,6- dimethylphenyl)thiazole-5- carboxamide | ¹H NMR (300 MHz, DMSO) δ 11.85 (s, 1H), 9.63 (s, 1H), 9.20 (s, 1H), 8.29 (s, 1H), 8.09- 7.86 (m, 3H), 7.71 (d, J = 7.6 Hz, 1H), 7.36 (m, 1H), 7.17 (d, J = 8.2 Hz, 1H), 6.91 (d, J = 8.2 Hz, 1H), 6.70 (d, J = 8.2 Hz, 1H), 2.10 (s, 3H), 2.02 (s, 3H). ¹⁹F NMR (282 MHz, DMSO) δ −109.05 (t, J = 8.8 Hz). MS (ES+) 453.9 m/z (M + H)⁺ |
| BAA- 316 | 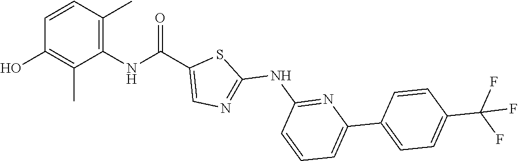 [SMILES: Cc1ccc(O)c(C)c1NC(=O)c1cnc(Nc2cccc(-c3ccc(C(F)(F)F)cc3)n2)s1] N-(3-hydroxy-2,6-dimethylphenyl)-2- ((6-(4-(trifluoromethyl)phenyl)pyridin- 2-yl)amino)thiazole-5-carboxamide | ¹H NMR (300 MHz, DMSO) δ 11.85 (br, 1H), 9.62 (s, 1H), 9.20 (s, 1H), 8.40 (d, J = 8.2 Hz, 2H), 8.28 (s, 1H), 7.99-7.83 (m, 3H), 7.67 (d, J = 7.5 Hz, 1H), 7.18 (d, J = 8.3 Hz, 1H), 6.91 (d, J = 8.2 Hz, 1H), 6.71 (d, J = 8.2 Hz, 1H), 2.10 (s, 3H), 2.02 (s, 3H). ¹⁹F NMR (282 MHz, DMSO) 0-61.07. MS (ES−) 483.4 m/z (M − H)⁻ |
| BAA- 317 | 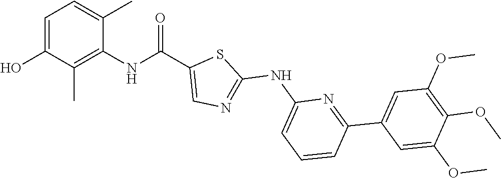 [SMILES: COc1cc(-c2cccc(Nc3ncc(C(=O)Nc4c(C)ccc(O)c4C)s3)n2)cc(OC)c1OC] N-(3-hydroxy-2,6-dimethylphenyl)-2- ((6-(3,4,5-trimethoxyphenyl)pyridin-2- yl)amino)thiazole-5-carboxamide | ¹H NMR (300 MHz, DMSO) δ 11.78 (s, 1H), 9.61 (s, 1H), 9.21 (s, 1H), 8.28 (s, 1H), 7.85 (t, J = 7.9 Hz, 1H), 7.67 (d, J = 7.6 Hz, 1H), 7.60 (s, 2H), 7.06 (d, J = 8.1 Hz, 1H), 6.91 (d, J = 8.2 Hz, 1H), 6.71 (d, J = 8.2 Hz, 1H), 3.93 (s, 6H), 3.72 (s, 3H), 2.09 (s, 3H), 2.01 (s, 3H). MS (ES−) 505.4 m/z (M − H)⁻ |
| BAA- 318 | 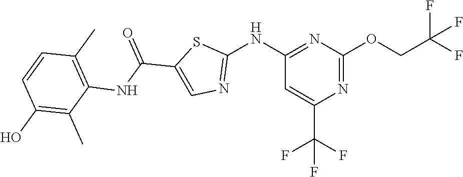 [SMILES: Cc1ccc(O)c(C)c1NC(=O)c1cnc(Nc2cc(C(F)(F)F)nc(OCC(F)(F)F)n2)s1] N-(3-hydroxy-2,6-dimethylphenyl)-2- ((2-(2,2,2-trifluoroethoxy)-6- (trifluoromethyl)pyrimidin-4- yl)amino)thiazole-5-carboxamide | ¹H NMR (300 MHz, Methanol-d4/chloroform- d) δ 8.22 (s, 1H), 7.05 (s, 1H), 6.86 (d, J = 8.3 Hz, 1H), 6.65 (d, J = 8.2 Hz, 1H), 4.97 (q, J = 8.3 Hz, 2H), 2.15 (s, 3H), 2.11 (s, 3H). ¹⁹F NMR (282 MHz, Methanol-d4/chloroform-d) δ −71.36 (3F), −74.54 (3F, t, J = 8.4 Hz). MS (ES+) 508.3 m/z (M + H)⁺ |
| BAA- 319 | 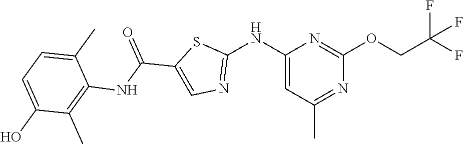 [SMILES: Cc1cc(Nc2ncc(C(=O)Nc3c(C)ccc(O)c3C)s2)nc(OCC(F)(F)F)n1] N-(3-hydroxy-2,6-dimethylphenyl)-2- ((6-methyl-2-(2,2,2- trifluoroethoxy)pyrimidin-4- yl)amino)thiazole-5-carboxamide | ¹H NMR (300 MHz, Methanol-d4/chloroform- d) δ 8.17 (s, 1H), 6.89 (d, J = 8.2 Hz, 1H), 6.68 (d, J = 8.2 Hz, 1H), 6.58 (d, J = 0.7 Hz, 1H), 4.98 (q, J = 8.4 Hz, 2H), 2.39 (s, 3H), 2.16 (s, 3H), 2.12 (s, 3H). ¹⁹F NMR (282 MHz, Methanol-d4/chloroform-d) δ −74.73 (t, J = 8.5 Hz). MS (ES+) 454.3 m/z (M + H)⁺ |
| BAA- 320 | 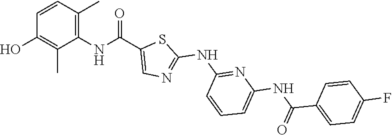 [SMILES: Cc1ccc(O)c(C)c1NC(=O)c1cnc(Nc2cccc(NC(=O)c3ccc(F)cc3)n2)s1] 2-((6-(4-fluorobenzamido)pyridin-2- yl)amino)-N-(3-hydroxy-2,6- dimethylphenyl)thiazole-5- carboxamide | ¹H NMR (300 MHz, Methanol-d4/chloroform- d) δ 8.15 (s, 1H), 8.09-7.97 (m, 2H), 7.86 (d, J = 7.9 Hz, 1H), 7.74 (t, J = 8.0 Hz, 1H), 7.20 (t, J = 8.6 Hz, 2H), 6.92 (d, J = 8.2 Hz, 1H), 6.84 (d, J = 8.0 Hz, 1H), 6.71 (d, J = 8.2 Hz, 1H), 2.19 (s, 3H), 2.15 (s, 3H). ¹⁹F NMR (282 MHz, Methanol-d4/chloroform-d) δ −107.78-−107.96 (m). MS (ES+) 478.3 m/z (M + H)⁺ |
| BAA- 321 | 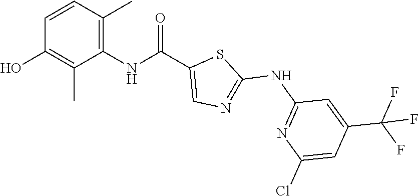 [SMILES: Cc1ccc(O)c(C)c1NC(=O)c1cnc(Nc2cc(C(F)(F)F)cc(Cl)n2)s1] 2-((6-chloro-4-(trifluoromethyl)pyridin- 2-yl)amino)-N-(3-hydroxy-2,6- dimethylphenyl)thiazole-5- carboxamide | ¹H NMR (300 MHz, Methanol-d4/chloroform- d) δ 8.12 (s, 1H), 7.18 (s, 1H), 7.11 (s, 1H), 6.88 (d, J = 8.3 Hz, 1H), 6.67 (d, J = 8.3 Hz, 1H), 2.15 (s, 3H), 2.11 (s, 3H). ¹⁹F NMR (282 MHz, Methanol-d4/chloroform-d) δ −65.46. MS (ES+) m/z 443.2 (M + H)⁺ |
| BAA- 322 | 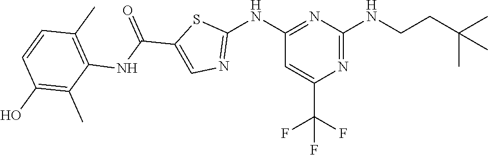 [SMILES: Cc1ccc(O)c(C)c1NC(=O)c1cnc(Nc2cc(C(F)(F)F)nc(NCCC(C)(C)C)n2)s1] 2-((2-((3,3-dimethylbutyl)amino)-6- (trifluoromethyl)pyrimidin-4-yl)amino)- N-(3-hydroxy-2,6- dimethylphenyl)thiazole-5- carboxamide | ¹H NMR (300 MHz, Methanol-d4/chloroform-d) δ 8.15 (s, 1H), 6.89 (d, J = 8.3 Hz, 1H), 6.68 (d, J = 8.2 Hz, 1H), 6.47 (s, 1H), 3.75-3.37 (m, 2H), 2.16 (s, 3H), 2.12 (s, 3H), 1.63- 1.46 (m, 2H), 0.97 (s, 9H). ¹⁹F NMR (282 MHz, Methanol-d4/chloroform-d) δ −72.03. MS (ES+) m/z 509.4 (M + H)⁺ |
| BAA- 323 | 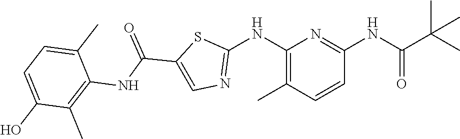 [SMILES: Cc1ccc(NC(=O)C(C)(C)C)nc1Nc1ncc(C(=O)Nc2c(C)ccc(O)c2C)s1] N-(3-hydroxy-2,6-dimethylphenyl)-2- ((3-methyl-6-pivalamidopyridin-2- yl)amino)thiazole-5-carboxamide | ¹H NMR (300 MHz, Methanol-d4/chloroform- d) δ 8.16 (s, 1H), 7.63 (d, J = 8.0 Hz, 1H), 7.52 (d, J = 7.9 Hz, 1H), 6.90 (d, J = 8.3 Hz, 1H), 6.69 (d, J = 8.2 Hz, 1H), 2.32 (s, 3H), 2.17 (s, 3H), 2.14 (s, 3H), 1.33 (s, 9H). MS (ES−) m/z 452.3 (M − H)⁻ |
| BAA- 324 | 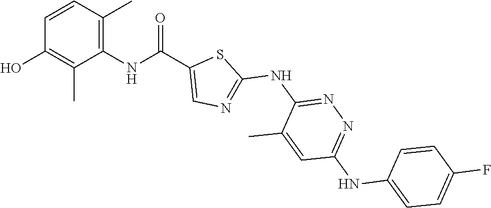 [SMILES: Cc1cc(Nc2ccc(F)cc2)nnc1Nc1ncc(C(=O)Nc2c(C)ccc(O)c2C)s1] 2-((6-((4-fluorophenyl)amino)-4- methylpyridazin-3-yl)amino)-N-(3- hydroxy-2,6-dimethylphenyl)thiazole- 5-carboxamide | ¹H NMR (300 MHz, Methanol-d4/chloroform- d) δ 8.12 (s, 1H), 7.53-7.42 (m, 2H), 7.04- 6.95 (m, 3H), 6.89 (d, J = 8.3 Hz, 1H), 6.68 (d, J = 8.2 Hz, 1H), 2.35 (s, 3H), 2.16 (s, 3H), 2.12 (s, 3H). ¹⁹F NMR (282 MHz, Methanol- d4/chloroform-d) δ −121.52-−121.75 (m). MS (ES−) m/z 463.3 (M − H)⁻ |
| BAA- 325 | 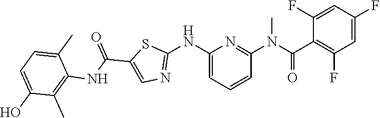 [SMILES: Cc1ccc(O)c(C)c1NC(=O)c1cnc(Nc2cccc(N(C)C(=O)c3c(F)cc(F)cc3F)n2)s1] N-(3-hydroxy-2,6-dimethylphenyl)-2- ((6-(2,4,6-trifluoro-N- methylbenzamido)pyridin-2- yl)amino)thiazole-5-carboxamide | ¹H NMR (300 MHz, Methanol-d4/chloroform- d) δ 8.14 (s, 1H), 7.60-7.40 (m, 1H), 6.95- 6.83 (m, 2H), 6.81-6.46 (m, 4H), 3.63 (s, 3H), 2.17 (s, 3H), 2.13 (s, 3H). ¹⁹F NMR (282 MHz, Methanol-d4/chloroform-d) δ −104.95 (p, J = 8.1 Hz), −109.11 (2F, br). MS (ES−) 526.3 m/z (M − H)⁻ |
| BAA- 326 | 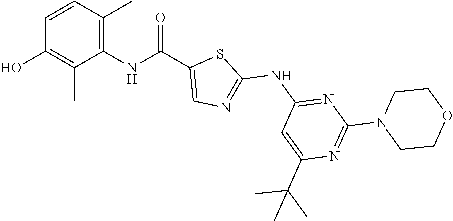 N-(3-hydroxy-2,6-dimethylphenyl)-2- ((2-morpholino-6- (trifluoromethyl)pyrimidin-4- yl)amino)thiazole-5-carboxamide | ¹H NMR (300 MHz, DMSO) δ 12.23 (br, 1H), 9.71 (s, 1H), 9.21 (s, 1H), 8.30 (s, 1H), 6.90 (d, J = 8.2 Hz, 1H), 6.69 (d, J = 8.2 Hz, 1H), 6.64 (s, 1H), 3.90-3.79 (m, 4H), 3.74-3.65 (m, 4H), 2.07 (s, 3H), 1.99 (s, 3H). ¹⁹F NMR (282 MHz, DMSO) 0-69.88. MS (ES+) m/z 495.2 (M + H)⁺ |
| BAA- 327 | 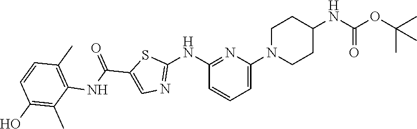 [SMILES: Cc1ccc(O)c(C)c1NC(=O)c1cnc(Nc2cccc(N3CCC(NC(=O)OC(C)(C)C)CC3)n2)s1] tert-butyl(1-(6-((5-((3-hydroxy-2,6- dimethylphenyl)carbamoyl)thiazol-2- yl)amino)pyridin-2-yl)piperidin-4- yl)carbamate | ¹H NMR (300 MHz, Methanol-d4/chloroform- d) for a pair of isomers δ 8.10 (s, 1H), 7.44 (m, 1H), 6.91 (d, J = 8.2 Hz, 1H), 6.70 (d, J = 8.2 Hz, 1H), 6.33-6.07 (m, 2H), 4.41-4.28 (m, 2H), 3.60 (m, 1H), 3.17-3.03 (m, 2H), 2.18 (s, 3H), 2.15 (s, 3H), 2.05-1.89 (m, 2H), 1.55-1.39 (m, 11H). MS (ES+) 539.3 m/z (M + H)⁺ |
| -BAA- 609 | 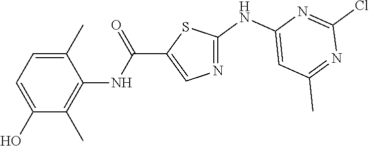 [SMILES: Cc1cc(Nc2ncc(C(=O)Nc3c(C)ccc(O)c3C)s2)nc(Cl)n1] 2-((2-chloro-6-methylpyrimidin-4- yl)amino)-N-(3-hydroxy-2,6- dimethylphenyl)thiazole-5- carboxamide | ¹H NMR (300 MHz, Methanol-d4/chloroform- d) δ 8.16 (s, 1H), 6.89 (d, J = 8.3 Hz, 1H), 6.79 (s, 1H), 6.68 (d, J = 8.2 Hz, 1H), 2.43 (s, 3H), 2.15 (s, 3H), 2.12 (s, 3H). MS (ES+) 390.1 m/z (M + H)⁺ |
| BAA- 434 | 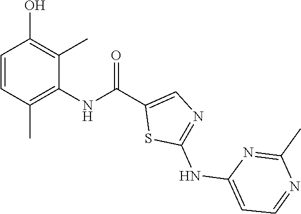 [SMILES: Cc1nccc(Nc2ncc(C(=O)Nc3c(C)ccc(O)c3C)s2)n1] N-(3-hydroxy-2,6-dimethylphenyl)-2- ((2-methylpyrimidin-4- yl)amino)thiazole-5-carboxamide | MS (ES+) 356.0 (M + H); ¹H NMR (DMSO-d6, 300 MHz) δ 11.98 (s, 1H), 9.64 (s, 1H), 9.18 (s, 1H), 8.40 (d, 1H, J = 5.8 Hz), 8.27 (s, 1H), 6.91-6.88 (m, 2H), 6.70 (d, 1H, J = 8.2 Hz), 2.59 (s, 3H), 2.08 (s, 3H), 2.00 (s, 3H) ppm |
| BAA- 435 | 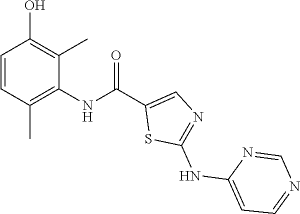 [SMILES: Cc1ccc(O)c(C)c1NC(=O)c1cnc(Nc2ccncn2)s1] N-(3-hydroxy-2,6-dimethylphenyl)-2- (pyrimidin-4-ylamino)thiazole-5- carboxamide | MS (ES+) 342.3 (M + H); ¹H NMR (DMSO-d6, 300 MHz) δ 12.07 (s, 1H), 9.67 (s, 1H), 9.18 (s, 1H), 8.91 (d, 1H, J = 1.0 Hz), 8.51 (d, 1H, J = 5.9 Hz), 8.27 (s, 1H), 7.11 (dd, 1H, J = 5.8, 1.2 Hz), 6.89 (d, 1H, J = 8.2 Hz), 6.69 (d, 1H, J = 8.2 Hz), 2.08 (s, 3H), 2.00 (s, 3H) ppm |
| BAA- 436 | 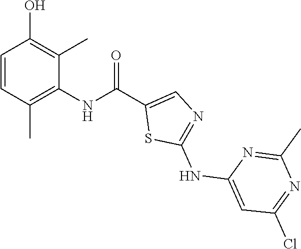 [SMILES: Cc1nc(Cl)cc(Nc2ncc(C(=O)Nc3c(C)ccc(O)c3C)s2)n1] 2-((6-chloro-2-methylpyrimidin-4- yl)amino)-N-(3-hydroxy-2,6- dimethylphenyl)thiazole-5- carboxamide | MS (ES+) 390.2 (M + H); ¹H NMR (DMSO-d6, 300 MHz) δ 12.18 (s, 1H), 9.69 (s, 1H), 9.19 (s, 1H), 8.28 (s, 1H), 6.94 (s, 1H), 6.90 (d, 1H, J = 8.2 Hz), 6.70 (d, 1H, J = 8.2 Hz), 2.59 (s, 3H), 2.08 (s, 3H), 2.00 (s, 3H) ppm |
| BAA- 437 | 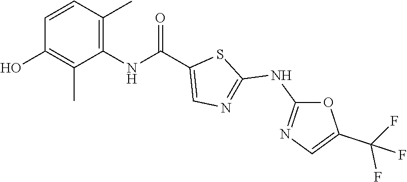 [SMILES: Cc1ccc(O)c(C)c1NC(=O)c1cnc(Nc2ncc(C(F)(F)F)o2)s1] N-(3- hydroxy-2,6-dimethylphenyl)-2-((5- (trifluoromethyl)oxazol-2- yl)amino)thiazole-5-carboxamide | ¹H NMR (300 MHz, DMSO) δ 12.98 (s, 1H), 9.68 (s, 1H), 9.21 (s, 1H), 8.43 (d, J = 2.0 Hz, 1H), 8.17 (s, 1H), 6.91 (d, J = 8.2 Hz, 1H), 6.71 (d, J = 8.2 Hz, 1H), 2.08 (s, 3H), 2.00 (s, 3H); ¹⁹F NMR (282 MHz, DMSO) δ −63.07; MS (ES+) m/z 399.2 (M + H)⁺. |
| BAA- 438 | 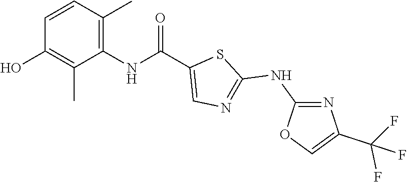 [SMILES: Cc1ccc(O)c(C)c1NC(=O)c1cnc(Nc2nc(C(F)(F)F)co2)s1] N-(3- hydroxy-2,6-dimethylphenyl)-2-((4- (trifluoro methyl)oxazol-2-yl)amino) thiazole-5-carboxamide | ¹H NMR (300 MHz, MeOD) δ 8.02 (q, J = 1.7 Hz, 1H), 8.01 (s, 1H), 6.92 (d, J = 8.9 Hz, 1H), 6.69 (d, J = 8.2 Hz, 1H), 2.15 (d, J = 0.6 Hz, 3H), 2.10 (s, 3H); MS (ES+) m/z 399.2 (M + H)⁺ |
| BAA- 439 | 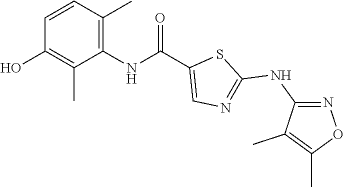 [SMILES: Cc1ccc(O)c(C)c1NC(=O)c1cnc(Nc2noc(C)c2C)s1] 2-((4,5- dimethylisoxazol-3-yl)amino)-N-(3- hydroxy-2,6-dimethylphenyl)thiazole- 5-carboxamide | ¹H NMR (300 MHz, MeOD) δ 8.09 (s, 1H), 6.92 (d, J = 8.3 Hz, 1H), 6.69 (d, J = 8.3 Hz, 1H), 2.32 (d, J = 0.8 Hz, 3H), 2.16 (s, 3H), 2.11 (s, 3H), 2.00 (d, J = 0.8 Hz, 3H); MS (ES+) m/z 359.2. |
| BAA- 440 | 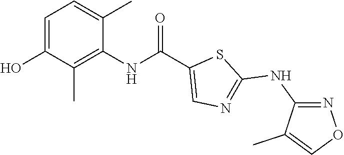 [SMILES: Cc1conc1Nc1ncc(C(=O)Nc2c(C)ccc(O)c2C)s1] N-(3- hydroxy-2,6-dimethylphenyl)-2-((4- methylisoxazol-3-yl)amino)thiazole-5- carboxamide | ¹H NMR (300 MHz, MeOD) δ 8.28 (d, J = 1.1 Hz, 1H), 8.10 (s, 1H), 6.92 (d, J = 8.2 Hz, 1H), 6.69 (d, J = 8.2 Hz, 1H), 2.66 (s, 1H), 2.16 (s, 3H), 2.12 (s, 3H), 2.08 (d, J = 1.1 Hz, 3H); MS (ES+) m/z 345.5 (M + H)⁺. |
| BAA- 441 | 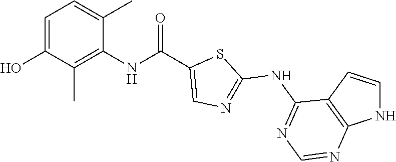 [SMILES: Cc1ccc(O)c(C)c1NC(=O)c1cnc(Nc2ncnc3[nH]ccc23)s1] 2-((7H- pyrrolo[2,3-d]pyrimidin-4-yl)amino)-N- (3-hydroxy-2,6- dimethylphenyl)thiazole-5- carboxamide | ¹H NMR (300 MHz, MeOD) δ 8.53 (s, 1H), 8.22 (s, 1H), 7.33 (d, J = 3.6 Hz, 1H), 6.97- 6.90 (m, 1H), 6.88 (d, J = 3.6 Hz, 1H), 6.70 (d, J = 8.3 Hz, 1H), 2.19 (s, 3H), 2.14 (s, 3H); MS (ES+) m/z 381.3 (M + H)⁺, |
| BAA- 442 | 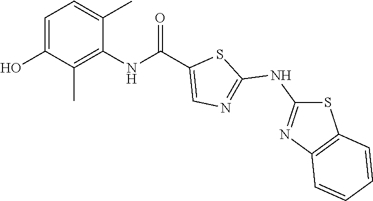 [SMILES: Cc1ccc(O)c(C)c1NC(=O)c1cnc(Nc2nc3ccccc3s2)s1] 2- (benzo[d]thiazol-2-ylamino)-N-(3- hydroxy-2,6-dimethyl phenyl)thiazole- 5-carboxamide | ¹H NMR (300 MHz, MeOD) δ 8.16 (s, 1H), 7.77 (d, J = 7.6 Hz, 1H), 7.66 (s, 1H), 7.47- 7.35 (m, 1H), 7.33-7.19 (m, 1H), 6.93 (d, J = 8.2 Hz, 1H), 6.70 (d, J = 8.2 Hz, 1H), 2.18 (s, 3H), 2.13 (s, 3H); MS (ES+) m/z 397.2 (M + H)⁺ |
| BAA- 443 | 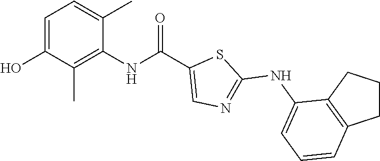 [SMILES: Cc1ccc(O)c(C)c1NC(=O)c1cnc(Nc2cccc3c2CCC3)s1] 2-((2,3-dihydro-1H-inden-4-yl)amino)- N-(3-hydroxy-2,6- dimethylphenyl)thiazole-5- carboxamide | ¹H NMR (300 MHz, MeOD) δ 7.95 (s, 1H), 7.49 (d, J = 8.8 Hz, 1H), 7.17 (t, J = 7.7 Hz, 1H), 7.07 (dd, J = 7.7, 1.0 Hz, 1H), 6.90 (d, J = 8.2 Hz, 1H), 6.68 (d, J = 8.2 Hz, 1H), 2.94 (m, 4H), 2.15 (s, 3H), 2.11 (m 2H), 2.10 (s, 3H); MS (ES+) m/z 380.3 (M + H)⁺. |
| BAA- 444 | 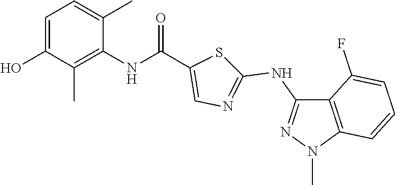 [SMILES: Cc1ccc(O)c(C)c1NC(=O)c1cnc(Nc2nn(C)c3cccc(F)c23)s1] 2-((4- fluoro-1-methyl-1H-indazol-3- yl)amino)-N-(3-hydroxy-2,6- dimethylphenyl)thiazole-5- carboxamide | ¹H NMR (300 MHz, MeOD) δ 8.10 (s, 1H), 7.39 (m, 1H), 7.29 (s, 1H), 6.91 (m, 1H), 6.84- 6.74 (m, 2H), 6.70 (s, 1H), 6.67 (s, 1H), 4.01 (s, 4H), 2.17 (s, 3H), 2.12 (s, 3H); ¹⁹F NMR (282 MHz, MeOD) δ −122.07; MS (ES+) m/z 412.6 (M + H)⁺. |
| BAA- 445 | 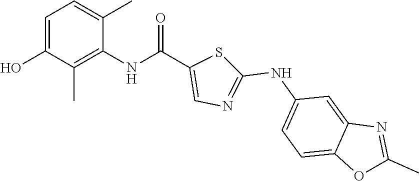 [SMILES: Cc1nc2cc(Nc3ncc(C(=O)Nc4c(C)ccc(O)c4C)s3)ccc2o1] N-(3- hydroxy-2,6-dimethylphenyl)-2-((2- methylbenzo[d]oxazol-5- yl)amino)thiazole-5-carboxamide | ¹H NMR (300 MHz, MeOD) δ 8.06 (d, J = 1.5 Hz, 1H), 8.01 (s, 1H), 7.53 (dd, J = 8.8, 0.7 Hz, 1H), 7.44 (dd, J = 8.8, 2.2 Hz, 1H), 6.91 (d, J = 8.2 Hz, 1H), 6.68 (d, J = 8.2 Hz, 1H), 2.63 (s, 3H), 2.16 (s, 3H), 2.11 (s, 3H); MS (ES+) m/z 395.3 (M + H)⁺. |
| BAA- 446 | 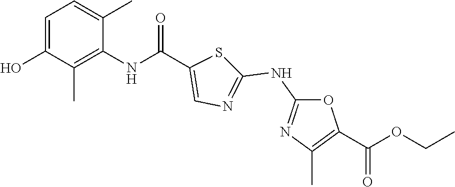 [SMILES: CCOC(=O)c1oc(Nc2ncc(C(=O)Nc3c(C)ccc(O)c3C)s2)nc1C] ethyl 2-((5-((3-hydroxy-2,6- dimethylphenyl)carbamoyl)thiazol-2- yl)amino)-4-methyloxazole-5- carboxylate | ¹H NMR (300 MHz, MeOD) δ 8.04 (s, 1H), 7.24 (s, 1H), 6.93 (d, J = 8.2 Hz, 1H), 6.70 (d, J = 8.2 Hz, 1H), 4.34 (q, J = 7.1 Hz, 2H), 2.46 (s, 3H), 2.17 (s, 3H), 2.12 (s, 3H), 1.38 (t, J = 7.1 Hz, 3H); MS (ES+) m/z 417.6 (M + H)⁺ |
| BAA- 447 | 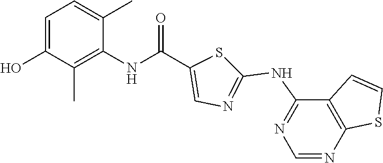 [SMILES: Cc1ccc(O)c(C)c1NC(=O)c1cnc(Nc2ncnc3sccc23)s1] N-(3- hydroxy-2,6-dimethylphenyl)-2- (thieno[2,3-d]pyrimidin-4- ylamino)thiazole-5-carboxamide | ¹H NMR (300 MHz, MeOD) δ 8.78 (s, 1H), 8.26 (s, 1H), 7.86 (d, J = 6.0 Hz, 1H), 7.72 (d, J = 6.0 Hz, 1H), 6.98-6.90 (m, 1H), 6.71 (d, J = 8.3 Hz, 1H), 2.19 (d, J = 0.6 Hz, 3H), 2.14 (s, 3H); MS (ES+) m/z 398.2 (M + H)⁺. |
| BAA- 448 | 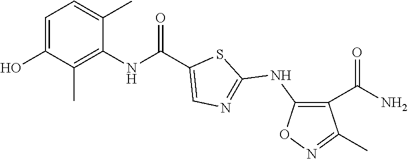 [SMILES: Cc1ccc(O)c(C)c1NC(=O)c1cnc(Nc2onc(C)c2C(N)=O)s1] 5-((5- ((3-hydroxy-2,6- dimethylphenyl)carbamoyl)thiazol-2- yl)amino)-3-methylisoxazole-4- carboxamide | 1H NMR (300 MHz, MeOD) δ 8.04 (s, 1H), 6.93 (d, J = 8.3 Hz, 1H), 6.70 (d, J = 8.0 Hz, 1H), 2.68 (s, 3H), 2.40 (s, 2H), 2.18 (s, 2H), 2.13 (s, 3H); MS (ES+) m/z 388.3 (M + H)⁺. |
| BAA- 449 | 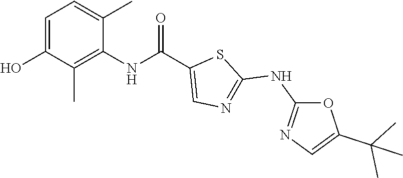 [SMILES: Cc1ccc(O)c(C)c1NC(=O)c1cnc(Nc2ncc(C(C)(C)C)o2)s1] 2-((5- (tert-butyl)oxazol-2-yl)amino)-N-(3- hydroxy-2,6-dimethylphenyl)thiazole- 5-carboxamide | ¹H NMR (300 MHz, MeOD) δ 8.09 (s, 1H), 6.92 (d, J = 8.3 Hz, 1H), 6.70 (d, J = 8.2 Hz, 1H), 6.05 (s, 1H), 2.17 (s, 3H), 2.12 (s, 3H), 1.38 (s, 9H); MS (ES+) m/z 387.3 (M + H)⁺. |
| BAA- 450 | 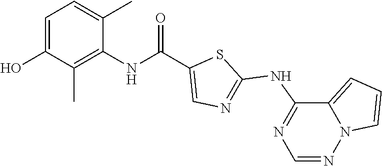 [SMILES: Cc1ccc(O)c(C)c1NC(=O)c1cnc(Nc2ncnn3cccc23)s1] N-(3- hydroxy-2,6-dimethylphenyl)-2- (pyrrolo[2, 1-f][1,2,4]triazin-4- ylamino)thiazole-5-carboxamide | ¹H NMR (300 MHz, DMSO) δ 12.75 (s, 1H), 9.73 (s, 1H), 9.19 (s, 1H), 8.35 (s, 1H), 8.29 (s, 1H), 7.94 (s, 1H), 7.43 (s, 1H), 6.96-6.80 (m, 2H), 6.70 (d, J = 8.2 Hz, 1H), 2.09 (s, 3H), 2.00 (s, 3H); MS (ES+) m/z 381.3 (M + H)⁺. |
| BAA- 451 | 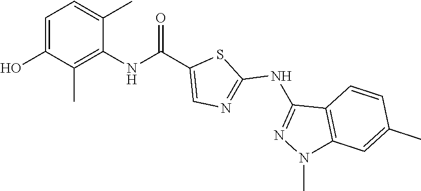 [SMILES: Cc1ccc2c(Nc3ncc(C(=O)Nc4c(C)ccc(O)c4C)s3)nn(C)c2c1] 2-((1,6- dimethyl-1H-indazol-3-yl)amino)-N-(3- hydroxy-2,6-dimethylphenyl)thiazole- 5-carboxamide | 1H NMR (300 MHz, DMSO) δ 11.80 (s, 1H), 9.54 (s, 1H), 9.17 (s, 1H), 8.19 (s, 1H), 7.96 (d, J = 8.3 Hz, 1H), 7.33 (s, 1H), 6.91 (dd, J = 13.6, 8.2 Hz, 2H), 6.69 (d, J = 8.2 Hz, 1H), 3.93 (s, 3H), 2.45 (s, 3H), 2.08 (s, 3H), 2.00 (s, 3H); MS (ES+) m/z 408.3 (M + H)⁺. |
| BAA- 452 | 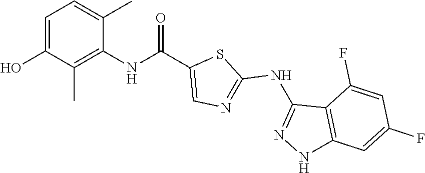 [SMILES: Cc1ccc(O)c(C)c1NC(=O)c1cnc(Nc2n[nH]c3cc(F)cc(F)c23)s1] 2- ((4,6-difluoro-1H-indazol-3-yl)amino)- N-(3-hydroxy-2,6- dimethylphenyl)thiazole-5- carboxamide | ¹H NMR (300 MHz, DMSO) δ 9.86 (s, 1H), 9.25 (s, 1H), 8.40 (s, 1H), 7.97 (dd, J = 9.2, 2.2 Hz, 1H), 7.21 (td, J = 10.1, 2.0 Hz, 1H), 6.92 (d, J = 8.2 Hz, 1H), 6.72 (d, J = 8.2 Hz, 1H), 2.56 (s, 1H), 2.10 (s, 3H), 2.02 (s, 3H); ¹⁹F NMR (282 MHz, DMSO) δ −106.54, −114.14;); MS (ES+) m/z 416.3 (M + H)⁺. |
| BAA- 453 | 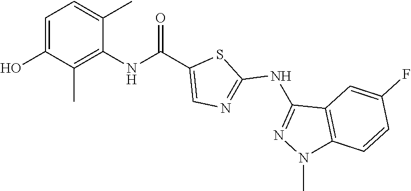 [SMILES: Cc1ccc(O)c(C)c1NC(=O)c1cnc(Nc2nn(C)c3ccc(F)cc23)s1] 2-((5- fluoro-1-methyl-1H-indazol-3- yl)amino)-N-(3-hydroxy-2,6- dimethylphenyl)thiazole-5- carboxamide | ¹H NMR (300 MHz, DMSO) δ 9.56 (s, 1H), 9.18 (s, 1H), 8.20 (s, 1H), 7.86 (d, J = 8.3 Hz, 1H), 7.70 (s, 1H), 7.60 (s, 1H), 7.39-7.32 (m, 1H), 6.89 (d, J = 7.9 Hz, 1H), 6.68 (d, J = 8.2 Hz, 1H), 3.98 (s, 3H), 2.08 (s, 3H), 1.99 (s, 3H); ¹⁹F NMR (282 MHz, MeOD) δ −123.92; MS (ES+) m/z 412.8 (M + H)⁺. |
| BAA- 455 | 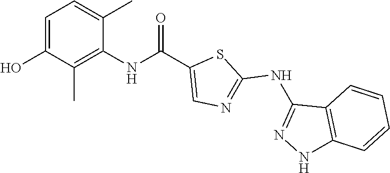 [SMILES: Cc1ccc(O)c(C)c1NC(=O)c1cnc(Nc2n[nH]c3ccccc23)s1] 2-((1H- indazol-3-yl)amino)-N-(3-hydroxy-2,6- dimethylphenyl)thiazole-5- carboxamide | ¹H NMR (300 MHz, DMSO) δ 9.76 (s, 1H), 9.22 (s, 1H), 8.37 (d, J = 6.6 Hz, 2H), 7.93 (d, J = 7.9 Hz, 1H), 7.66-7.54 (m, 1H), 7.35- 7.28 (m, 1H), 6.90 (d, J = 8.2 Hz, 1H), 6.70 (d, J = 8.2 Hz, 2H), 2.09 (s, 3H), 2.01 (s, 3H); MS (ES+) m/z 380.3 (M + H)⁺. |
| BAA- 456 | 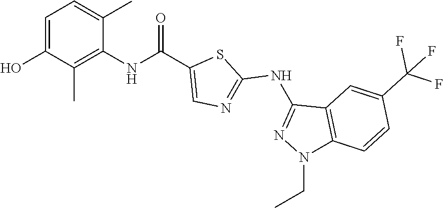 [SMILES: CCn1nc(Nc2ncc(C(=O)Nc3c(C)ccc(O)c3C)s2)c2cc(C(F)(F)F)ccc21] 2-((1- ethyl-5-(trifluoromethyl)-1H-indazol-3- yl)amino)-N-(3-hydroxy-2,6- dimethylphenyl)thiazole-5- carboxamide | ¹H NMR (300 MHz, DMSO) δ 12.09 (s, 1H), 9.62 (s, 1H), 9.22 (s, 1H), 8.66 (s, 1H), 8.25 (s, 1H), 7.84 (d, J = 9.0 Hz, 1H), 7.72 (d, J = 10.8 Hz, 1H), 6.91 (d, J = 8.2 Hz, 1H), 6.71 (d, J = 8.2 Hz, 1H), 4.45 (q, J = 7.0 Hz, 2H), 2.10 (s, 3H), 2.02 (s, 3H), 1.45 (t, J = 7.2 Hz, 3H); ¹⁹F NMR (282 MHz, DMSO) δ −59.13; MS (ES+) m/z 476.3 (M + H)⁺. |
| BAA- 457 | 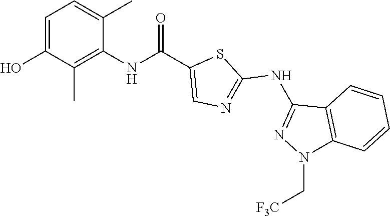 [SMILES: Cc1ccc(O)c(C)c1NC(=O)c1cnc(Nc2nn(CC(F)(F)F)c3ccccc23)s1] N-(3- hydroxy-2,6-dimethylphenyl)-2-((1- (2,2,2-trifluoroethyl)-1H-indazol-3- yl)amino)thiazole-5-carboxamide | ¹H NMR (300 MHz, MeOD) δ 8.15 (s, 1H), 8.00 (d, J = 8.2 Hz, 1H), 7.60-7.49 (m, 2H), 7.28-7.21 (m, 1H), 6.93 (d, J = 8.2 Hz, 1H), 6.70 (d, J = 8.2 Hz, 1H), 5.13 (q, J = 8.8 Hz, 2H), 2.19 (s, 3H), 2.13 (s, 3H); ¹⁹F NMR (282 MHz, MeOD) δ −71.94; MS (ES+) m/z 462.2 (M + H)⁺ |
| BAA- 458 | 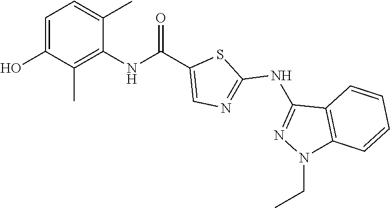 [SMILES: CCn1nc(Nc2ncc(C(=O)Nc3c(C)ccc(O)c3C)s2)c2ccccc21] 2-((1- ethyl-1H-indazol-3-yl)amino)-N-(3- hydroxy-2,6-dimethylphenyl)thiazole- 5-carboxamide | ¹H NMR (300 MHz, MeOD) δ 8.14 (s, 1H), 7.96-7.89 (m, 1H), 7.50-7.40 (m, 2H), 7.14 (ddd, J = 7.7, 6.4, 1.4 Hz, 1H), 6.93 (d, J = 8.2 Hz, 1H), 6.70 (d, J = 8.3 Hz, 1H), 4.40 (q, J = 7.2 Hz, 2H), 2.19 (s, 3H), 2.14 (s, 3H), 1.51 (t, J = 7.2 Hz, 3H); MS (ES+) m/z 408.3 (M + H)⁺. |
| BAA- 459 | 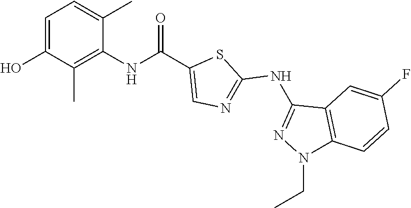 [SMILES: CCn1nc(Nc2ncc(C(=O)Nc3c(C)ccc(O)c3C)s2)c2cc(F)ccc21] 2-((1- ethyl-5-fluoro-1H-indazol-3-yl)amino)- N-(3-hydroxy-2,6- dimethylphenyl)thiazole-5- carboxamide | ¹H NMR (300 MHz, MeOD) δ 8.15 (s, 1H), 7.64 (dd, J = 9.0, 3.2 Hz, 1H), 7.53 (dd, J = 9.2, 4.1 Hz, 1H), 7.28 (td, J = 9.1, 2.4 Hz, 1H), 6.95 (d, J = 8.3 Hz, 1H), 6.72 (d, J = 8.2 Hz, 1H), 4.41 (q, J = 7.2 Hz, 2H), 2.20 (s, 3H), 2.15 (s, 3H), 1.53 (t, J = 7.2 Hz, 3H); ¹⁹F NMR (282 MHz, MeOD) δ −125.84; MS (ES+) m/z 426.3 (M + H)⁺. |
| BAA- 460 | 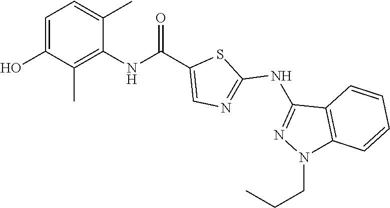 [SMILES: CCCn1nc(Nc2ncc(C(=O)Nc3c(C)ccc(O)c3C)s2)c2ccccc21] N-(3- hydroxy-2,6-dimethylphenyl)-2-((1- propyl-1H-indazol-3-yl)amino)thiazole- 5-carboxamide | ¹H NMR (300 MHz, DMSO) δ 11.88 (s, 1H), 9.55 (s, 1H), 9.18 (s, 1H), 8.21 (s, 1H), 8.10 (d, J = 8.0 Hz, 1H), 7.59 (d, J = 7.7 Hz, 1H), 7.48-7.36 (m, 1H), 7.16-7.04 (m, 1H), 6.89 (d, J = 8.1 Hz, 1H), 6.69 (d, J = 8.2 Hz, 1H), 4.29 (t, J = 6.8 Hz, 2H), 2.08 (s, 3H), 2.00 (s, 3H), 1.88 (q, J = 7.1 Hz, 2H), 0.85 (t, J = 7.4 Hz, 3H); MS (ES+) m/z 422.4 (M + H)⁺. |
| BAA- 461 | 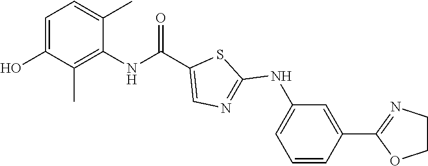 [SMILES: Cc1ccc(O)c(C)c1NC(=O)c1cnc(Nc2cccc(C3=NCCO3)c2)s1] 2-((3- (4,5-dihydrooxazol-2- yl)phenyl)amino)-N-(3-hydroxy-2,6- dimethylphenyl)thiazole-5- carboxamide | ¹H NMR (300 MHz, MeOD) δ 8.22 (t, J = 1.8 Hz, 1H), 8.04 (s, 1H), 7.80 (ddd, J = 8.2, 2.4, 1.0 Hz, 1H), 7.64-7.58 (m, 1H), 7.44 (t, J = 8.2 Hz, 1H), 6.92 (d, J = 8.2 Hz, 1H), 6.69 (d, J = 8.2 Hz, 1H), 4.60-4.47 (m, 2H), 4.05 (m, 2H), 2.17 (s, 3H), 2.12 (s, 3H). MS (ES+) m/z 409.3 (M + H)⁺. |
| Interm ediate- | 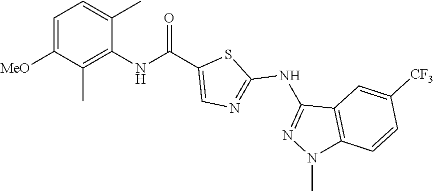 [SMILES: COc1ccc(C)c(NC(=O)c2cnc(Nc3nn(C)c4ccc(C(F)(F)F)cc34)s2)c1C] N-(3-methoxy-2,6-dimethylphenyl)-2- ((1-methyl-5-(trifluoromethyl)-1H- indazol-3-yl)amino)thiazole-5- carboxamide | ¹H NMR (300 MHz, DMSO) δ 12.08 (s, 1H), 9.68 (s, 1H), 8.65 (s, 1H), 8.25 (s, 1H), 7.79 (d, J = 8.9 Hz, 1H), 7.74-7.69 (m, 1H), 7.49- 7.34 (m, 1H), 7.09 (d, J = 8.4 Hz, 1H), 6.86 (d, J = 8.5 Hz, 1H), 4.05 (s, 3H), 3.79 (s, 3H), 2.14 (s, 3H), 2.04 (s, 3H); ¹⁹F NMR (282 MHz, DMSO) δ 58.95. MS (ES+) m/z 476.3 (M + H)⁺. |
| BAA- 257 | 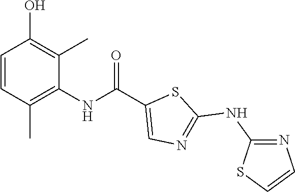 [SMILES: Cc1ccc(O)c(C)c1NC(=O)c1cnc(Nc2nccs2)s1] N-(3-hydroxy-2,6-dimethylphenyl)-2- (thiazol-2-ylamino)thiazole-5- carboxamide | ¹H NMR (300 MHz, DMSO-d6) δ 12.48 (s, 1H), 9.61 (s, 1H), 9.17 (s, 1H), 8.17 (s, 1H), 7.42 (d, J = 3.8 Hz, 1H), 7.10 (s, 1H), 6.89 (d, J = 8.2 Hz, 1H), 6.69 (d, J = 8.2 Hz, 1H), 2.07 (s, 3H), 1.99 (s, 3H). MS (ES+) m/z 347.2 (M + H). |
| BAA- 258 | 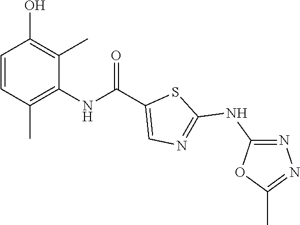 [SMILES: Cc1nnc(Nc2ncc(C(=O)Nc3c(C)ccc(O)c3C)s2)o1] N-(3-hydroxy-2,6-dimethyl phenyl)-2- ((5-methyl-1,3,4-oxadiazol-2- yl)amino)thiazole-5-carboxamide | ¹H NMR (300 MHz, DMSO-d6) δ 12.90 (s, 1H), 9.63 (s, 1H), 9.19 (s, 1H), 8.13 (s, 1H), 6.89 (d, J = 8.2 Hz, 1H), 6.69 (d, J = 8.2 Hz, 1H), 2.40 (s, 3H), 2.06 (s, 3H), 1.98 (s, 3H). MS (ES+) m/z 346.4 (M + H). |
| BAA- 262 | 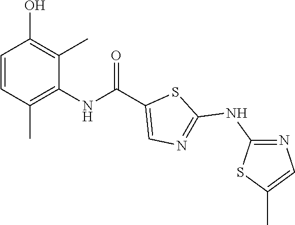 [SMILES: Cc1cnc(Nc2ncc(C(=O)Nc3c(C)ccc(O)c3C)s2)s1] N-(3-hydroxy-2,6-dimethyl phenyl)-2- ((5-methylthiazol-2-yl)amino)thiazole- 5-carboxamide | ¹H NMR (300 MHz, DMSO-d6) δ 12.27 (s, 1H), 9.58 (s, 1H), 9.16 (s, 1H), 8.15 (s, 1H), 7.09 (d, J = 1.5 Hz, 1H), 6.88 (d, J = 8.2 Hz, 1H), 6.68 (d, J = 8.2 Hz, 1H), 2.32 (d, J = 1.3 Hz, 3H), 2.06 (s, 3H), 1.98 (s, 3H). MS (ES+) m/z 361.2 (M + H). |
| BAA- 263 | 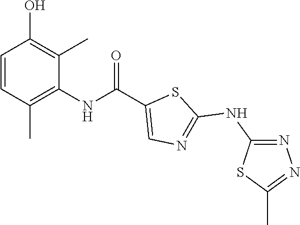 [SMILES: Cc1nnc(Nc2ncc(C(=O)Nc3c(C)ccc(O)c3C)s2)s1] N-(3-hydroxy-2,6-dimethyl phenyl)-2- ((5-methyl-1,3,4-thiadiazol-2- yl)amino)thiazole-5-carboxamide | ¹H NMR (300 MHz, MeOD) δ 8.10 (s, 1H), 6.92 (dt, J = 8.3, 0.7 Hz, 1H), 6.69 (d, J = 8.2 Hz, 1H), 2.63 (s, 3H), 2.17-2.14 (m, 3H), 2.11 (s, 3H). MS (ES+) m/z 362.4 (M + H). |
| BAA- 264 | 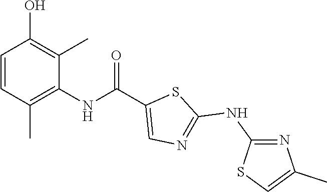 [SMILES: Cc1csc(Nc2ncc(C(=O)Nc3c(C)ccc(O)c3C)s2)n1] N-(3-hydroxy-2,6-dimethyl phenyl)-2- ((4-methylthiazol-2-yl)amino)thiazole- 5-carboxamide | ¹H NMR (300 MHz, DMSO-d6) δ 12.39 (s, 1H), 9.60 (s, 1H), 9.17 (s, 1H), 8.17 (s, 1H), 6.89 (d, J = 8.2 Hz, 1H), 6.69 (d, J = 8.2 Hz, 1H), 6.62 (s, 1H), 2.29-2.19 (m, 3H), 2.07 (s, 3H), 1.99 (s, 3H). MS (ES+) m/z 361.2 (M + H). |
| BAA- 265 | 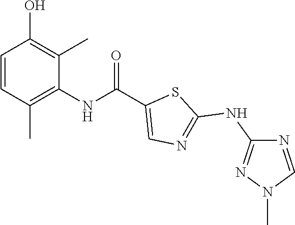 [SMILES: Cc1ccc(O)c(C)c1NC(=O)c1cnc(Nc2ncn(C)n2)s1] N-(3-hydroxy-2,6-dimethyl phenyl)-2- ((1-methyl-1H-1,2,4-triazol-3- yl)amino)thiazole-5-carboxamide | ¹H NMR (300 MHz, DMSO-d6) δ 11.64 (s, 1H), 9.53 (s, 1H), 9.15 (s, 1H), 8.36 (d, J = 0.8 Hz, 1H), 8.12 (s, 1H), 6.88 (d, J = 8.2 Hz, 1H), 6.68 (d, J = 8.2 Hz, 1H), 3.91-3.74 (m, 3H), 2.06 (s, 3H), 1.98 (s, 3H). MS (ES+) m/z 345.2 (M + H). |
| BAA- 266 | 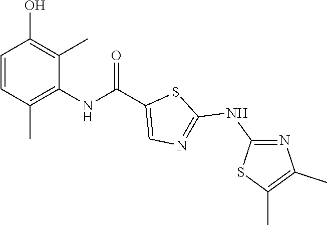 [SMILES: Cc1ccc(O)c(C)c1NC(=O)c1cnc(Nc2nc(C)c(C)s2)s1] 2-((4,5-dimethylthiazol-2-yl)amino)-N- (3-hydroxy-2,6- dimethylphenyl)thiazole-5- carboxamide | 1H NMR (300 MHz, DMSO-d6) δ 12.18 (s, 1H), 9.57 (s, 1H), 9.16 (s, 1H), 8.15 (s, 1H), 6.88 (d, J = 8.2 Hz, 1H), 6.68 (d, J = 8.2 Hz, 1H), 2.20 (s, 3H), 2.14 (s, 3H), 2.06 (s, 3H), 1.98 (s, 3H). MS (ES+) m/z 375.2 (M + H). |
| BAA- 505 | 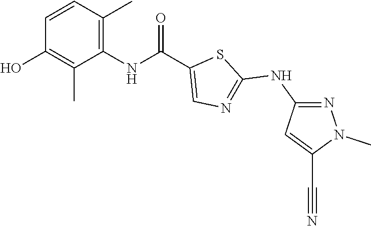 [SMILES: Cc1ccc(O)c(C)c1NC(=O)c1cnc(Nc2cc(C#N)n(C)n2)s1] 2-((5-cyano-1-methyl-1H-pyrazol-3- yl)amino)-N-(3-hydroxy-2,6- dimethylphenyl)thiazole-5- carboxamide | ¹H NMR (400 MHz, DMSO-d6) δ 11.29 (s, 1H), 9.54 (s, 1H), 9.14 (s, 1H), 8.08 (s, 1H), 6.84 (d, J = 8.2 Hz, 1H), 6.81 (s, 1H), 6.64 (d, J = 8.2 Hz, 1H), 3.92 (s, 3H), 2.02 (s, 3H), 1.94 (s, 3H). MS (ES+) m/z 369.2 (M + H)⁺. |
| BAA- 496 | 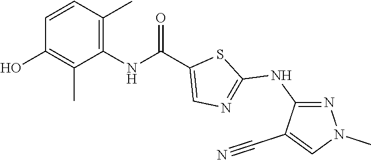 [SMILES: Cc1ccc(O)c(C)c1NC(=O)c1cnc(Nc2nn(C)cc2C#N)s1] 2-((4-cyano-1-methyl-1H-pyrazol-3- yl)amino)-N-(3-hydroxy-2,6- dimethylphenyl)thiazole-5- carboxamide | ¹H NMR (400 MHz, DMSO-d6) δ 9.49 (s, 1H), 9.17 (s, 1H), 8.34 (s, 1H), 8.04 (s, 1H), 6.84 (d, J = 8.2 Hz, 1H), 6.64 (d, J = 8.1 Hz, 1H), 3.80 (s, 3H), 2.02 (s, 3H), 1.94 (s, 3H). MS (ES+) m/z 369.2 (M + H)⁺. |
| BAA- 573 | 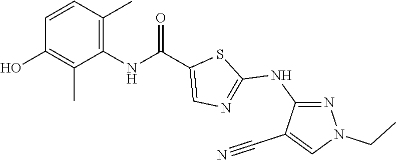 [SMILES: CCn1cc(C#N)c(Nc2ncc(C(=O)Nc3c(C)ccc(O)c3C)s2)n1] 2-((4-cyano-1-ethyl-1H-pyrazol-3- yl)amino)-N-(3-hydroxy-2,6- dimethylphenyl)thiazole-5- carboxamide | ¹H NMR (400 MHz, DMSO-d6) δ 10.92 (br s, 1H), 10.39 (br s, 1H), 10.27 (s, 1H), 9.33 (s, 1H), 9.01 (s, 1H), 8.84 (br s, 1H), 6.93 (d, J = 8.2 Hz, 1H), 6.74 (d, J = 8.2 Hz, 1H), 4.48 (br q, J = 8.5 Hz, 2H), 2.09 (s, 3H), 2.01 (s, 3H), 1.50 (t, J = 7.3 Hz, 3H). MS (ES+) m/z 381.2 (M + H)⁺ |
| BAA- 467 | 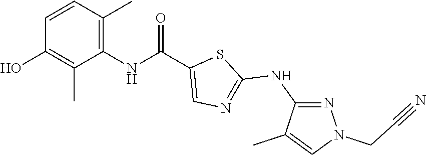 [SMILES: Cc1cn(CC#N)nc1Nc1ncc(C(=O)Nc2c(C)ccc(O)c2C)s1] 2-((1-(cyanomethyl)-4-methyl-1H- pyrazol-3-yl)amino)-N-(3-hydroxy-2,6- dimethylphenyl)thiazole-5- carboxamide | ¹H NMR (400 MHz, DMSO-d6) δ 10.90 (s, 1H), 9.51 (s, 1H), 9.17 (s, 1H), 8.13 (s, 1H), 7.58 (d, J = 1.0 Hz, 1H), 6.88 (d, J = 8.2 Hz, 1H), 6.68 (d, J = 8.2 Hz, 1H), 5.36 (s, 2H), 2.07 (s, 3H), 2.01 (s, 3H), 1.99 (s, 3H). MS (ES+) m/z 383.1 (M + H)⁺. |
| BAA- 518 | 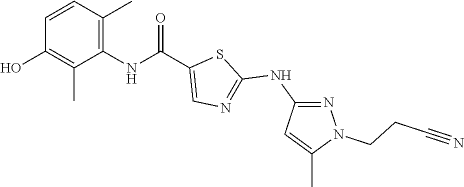 [SMILES: Cc1ccc(O)c(C)c1NC(=O)c1cnc(Nc2cc(C)n(CCC#N)n2)s1] 2-((1-(2-cyanoethyl)-5-methyl-1H- pyrazol-3-yl)amino)-N-(3-hydroxy-2,6- dimethylphenyl)thiazole-5- carboxamide | ¹H NMR (400 MHz, DMSO-d6) δ 10.98 (s, 1H), 9.46 (s, 1H), 9.19 (s, 1H), 8.09 (s, 1H), 6.88 (d, J = 8.2 Hz, 1H), 6.68 (d, J = 8.1 Hz, 1H), 5.82 (s, 1H), 4.25 (t, J = 6.4 Hz, 2H), 3.01 (t, J = 6.3 Hz, 2H), 2.29 (s, 3H), 2.06 (s, 3H), 1.98 (s, 3H). MS (ES+) m/z 397.4 (M + H)⁺. |
| BAA- 596 | 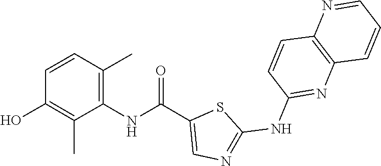 [SMILES: Cc1ccc(O)c(C)c1NC(=O)c1cnc(Nc2ccc3ncccc3n2)s1] 2-((1,5-naphthyridin-2-yl)amino)-N-(3- hydroxy-2,6-dimethylphenyl)thiazole- 5-carboxamide | ¹H NMR (400 MHz, DMSO-d6) δ 12.19 (s, 1H), 9.64 (s, 1H), 9.20 (s, 1H), 8.79 (dd, J = 4.2, 1.6 Hz, 1H), 8.32 (d, J = 10.8 Hz, 2H), 8.27 (dd, J = 8.5, 1.6 Hz, 1H), 7.71 (dd, J = 8.5, 4.2 Hz, 1H), 7.53 (d, J = 9.1 Hz, 1H), 6.91 (d, J = 8.2 Hz, 1H), 6.70 (d, J = 8.2 Hz, 1H), 2.10 (s, 3H), 2.02 (s, 3H). MS (ES+) m/z 392.2 (M + H)⁺. |
| BAA- 597 | 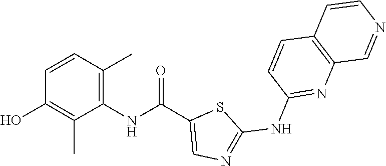 [SMILES: Cc1ccc(O)c(C)c1NC(=O)c1cnc(Nc2ccc3ccncc3n2)s1] 2-((1,7-naphthyridin-2-yl)amino)-N-(3- hydroxy-2,6-dimethylphenyl)thiazole- 5-carboxamide | ¹H NMR (400 MHz, DMSO-d6) δ 12.24 (s, 1H), 9.65 (s, 1H), 9.22 (d, J = 9.3 Hz, 2H), 8.49 (d, J = 5.4 Hz, 1H), 8.37-8.29 (m, 2H), 7.81 (d, J = 5.4 Hz, 1H), 7.52 (d, J = 8.9 Hz, 1H), 6.91 (d, J = 8.2 Hz, 1H), 6.71 (d, J = 8.2 Hz, 1H), 2.10 (s, 3H), 2.02 (s, 3H). MS (ES+) m/z 392.2 (M + H)⁺. |
| BAA- 602 | 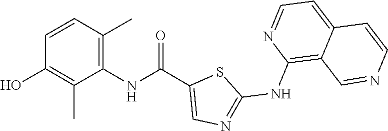 [SMILES: Cc1ccc(O)c(C)c1NC(=O)c1cnc(Nc2nccc3ccncc23)s1] 2-((2,7-naphthyridin-1-yl)amino)-N-(3- hydroxy-2,6-dimethylphenyl)thiazole- 5-carboxamide | ¹H NMR (400 MHz, DMSO-d6) δ 11.05 (s, 1H), 9.69 (s, 1H), 9.18 (s, 1H), 9.04 (d, J = 3.8 Hz, 1H), 8.45 (d, J = 8.3 Hz, 1H), 8.34 (d, J = 6.7 Hz, 2H), 7.89 (dd, J = 8.4, 4.3 Hz, 1H), 7.51 (d, J = 5.8 Hz, 1H), 6.90 (d, J = 8.3 Hz, 1H), 6.70 (d, J = 8.2 Hz, 1H), 2.10 (s, 3H), 2.02 (s, 3H). MS (ES+) m/z 392.2 (M + H)⁺. |
| BAA- 603 | 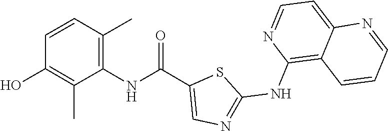 [SMILES: Cc1ccc(O)c(C)c1NC(=O)c1cnc(Nc2nccc3ncccc23)s1] 2-((1,6-naphthyridin-5-yl)amino)-N-(3- hydroxy-2,6-dimethylphenyl)thiazole- 5-carboxamide | ¹H NMR (400 MHz, DMSO-d6) δ 10.45 (s, 1H), 9.82 (s, 1H), 9.21 (s, 1H), 8.85 (d, J = 8.2 Hz, 1H), 8.82 (dd, J = 4.7, 1.5 Hz, 1H), 8.80-8.74 (m, 1H), 8.47 (s, 1H), 7.55 (dd, J = 8.1, 4.7 Hz, 1H), 6.90 (d, J = 8.2 Hz, 1H), 6.71 (d, J = 8.2 Hz, 1H), 6.64 (d, J = 8.2 Hz, 1H), 2.09 (s, 3H), 2.07 (s, 3H), 2.01 (s, 3H). MS (ES+) m/z 392.2 (M + H)⁺. |
| BAA- 604 | 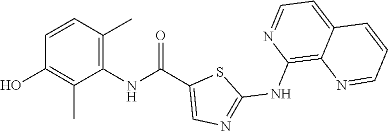 [SMILES: Cc1ccc(O)c(C)c1NC(=O)c1cnc(Nc2nccc3cccnc23)s1] 2-((1,7-naphthyridin-8-yl)amino)-N-(3- hydroxy-2,6-dimethylphenyl)thiazole- 5-carboxamide | ¹H NMR (400 MHz, DMSO-d6) δ 9.82 (s, 1H), 9.62 (s, 1H), 9.21 (s, 1H), 8.72 (dd, J = 11.2, 6.6 Hz, 2H), 8.46 (s, 1H), 7.55 (d, J = 5.2 Hz, 1H), 6.90 (d, J = 8.1 Hz, 1H), 6.70 (d, J = 8.2 Hz, 1H), 6.60 (d, J = 8.0 Hz, 1H), 2.09 (s, 3H), 2.07 (s, 3H), 2.01 (s, 3H). MS (ES+) m/z 392.1 (M + H)⁺. |

### N-(3-Hydroxy-2,6-dimethylphenyl)-2-((6-methoxypyrazolo[1,5-a]pyridin-2-yl)amino)thiazole-5-carboxamide( ) BAA-242

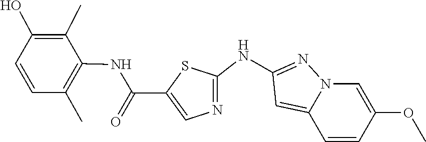 [SMILES: COc1ccc2cc(Nc3ncc(C(=O)Nc4c(C)ccc(O)c4C)s3)nn2c1]

Step 1:6-methoxypyrazolo[1,5-a]pyridin-2-amine was prepared according to Nishigaya, Y et al, Tetrahedron Letters 2014, 55 (43), 5963-5966.

Step 2:2-bromo-N-(3-hydroxy-2,6-dimethyl-phenyl)thiazole-5-carboxamide (100 mg, 0.306 mmol, 1 eq), tris(dibenzylideneacetone) dipalladium (0)(28 mg, 0.0306 mmol, 0.1 eq),cesium carbonate (200 mg, 0.611 mmol, 2.00 eq) and 6-methoxypyrazolo[1,5-a]pyridin-2-amine (100 mg, 0.611 mmol, 2 eq) in 1,4-dioxane (4 mL) and water (1 mL) was degassed under nitrogen. 4,5-bis(diphenylphospheno)-9,9-dimethylxanthene (35 mg, 0.0611 mmol, 0.2 eq) was added and the reaction degassed again, then heated to 90° C. for 18 h. The reaction was partitioned between EtOAc and water. The organic phase was washed with brine, dried (MgSO₄) and concentrated in vacuo. Column chromatography (CH2Cl2: methanol) followed by reverse phase column chromatography (water:acetonitrile) gave N-(3-hydroxy-2,6-dimethyl-phenyl)-2-[(6-methoxypyrazolo[1,5-a]pyridin-2-yl)amino]thiazole-5-carboxamide (25 mg, 0.0611 mmol, 20%). MS (ES+) m/z 410 (M+H). ¹H NMR (300 MHz, Methanol-d4) δ 8.07 (d, J=9.6 Hz, 2H), 7.75 (d, J=23.0 Hz, 1H), 7.35 (d, J=9.5 Hz, 1H), 6.98 (dd, J=9.5, 2.3 Hz, 1H), 6.91 (d, J=8.4 Hz, 1H), 6.69 (d, J=8.2 Hz, 1H), 6.24 (d, J=2.4 Hz, 1H), 3.84 (d, J=2.5 Hz, 3H), 2.17 (s, 3H), 2.13 (s, 3H).

### N-(3-Hydroxy-2,6-dimethylphenyl)-2-((6-hydroxypyrazolo[1,5-a]pyridin-2-yl)amino)thiazole-5-carboxamide( ) BAA-343

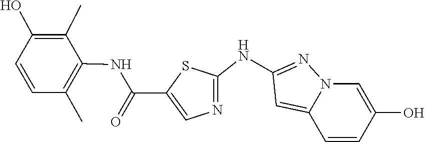 [SMILES: Cc1ccc(O)c(C)c1NC(=O)c1cnc(Nc2cc3ccc(O)cn3n2)s1]

To N-(3-hydroxy-2,6-dimethyl-phenyl)-2-[(6-methoxypyrazolo[1,5-a]pyridin-2-yl)amino]thiazole-5-carboxamide (21 mg, 0.0513 mmol, 1.00 eq) in DCM (4 mL) at 0° C. was added dropwise boron tribromide solution (1M in DCM, 0.28 mL, 0.282 mmol, 5.5 eq). The reaction was allowed to warm to rt over 2 h and stirred at rt for a further 48 h. The reaction was concentrated in vacuo, then quenched by the addition of NaHCO₃ (sat. aqueous)(7 mL). EtOAc (5 mL) and water (5 mL) were added. The phases were separated and the aqueous phase extracted with EtOAc (2×5 mL). Combined organic phases were washed with brine (20 mL), dried over MgSO4, filtered and concentrated in vacuo. Column chromatography (dichloromethane:methanol) gave N-(3-hydroxy-2,6-dimethyl-phenyl)-2-[(6-hydroxypyrazolo[1,5-a]pyridin-2-yl)amino]thiazole-5-carboxamide (5.4 mg, 0.0137 mmol, 27%). MS (ES+) m/z 396 (M+H). ¹H NMR (300 MHz, Methanol-d4) δ 8.06 (s, 1H), 8.01 (dt, J=1.9, 0.9 Hz, 1H), 7.36 (dd, J=9.5, 0.9 Hz, 1H), 6.97 (dd, J=9.5, 2.1 Hz, 1H), 6.92 (d, J=8.3 Hz, 1H), 6.69 (d, J=8.2 Hz, 1H), 6.23 (d, J=0.9 Hz, 1H), 2.17 (s, 3H), 2.12 (s, 3H).

### N-(3-Hydroxy-2.6-dimethyl-phenyl)-2-[[5-[[4-(2-hydroxyethyl) piperazin-4-ium-1-yl]methyl]-1-methyl-pyrazol-3-yl]amino]thiazole-5-carboxamide (BAA-052)

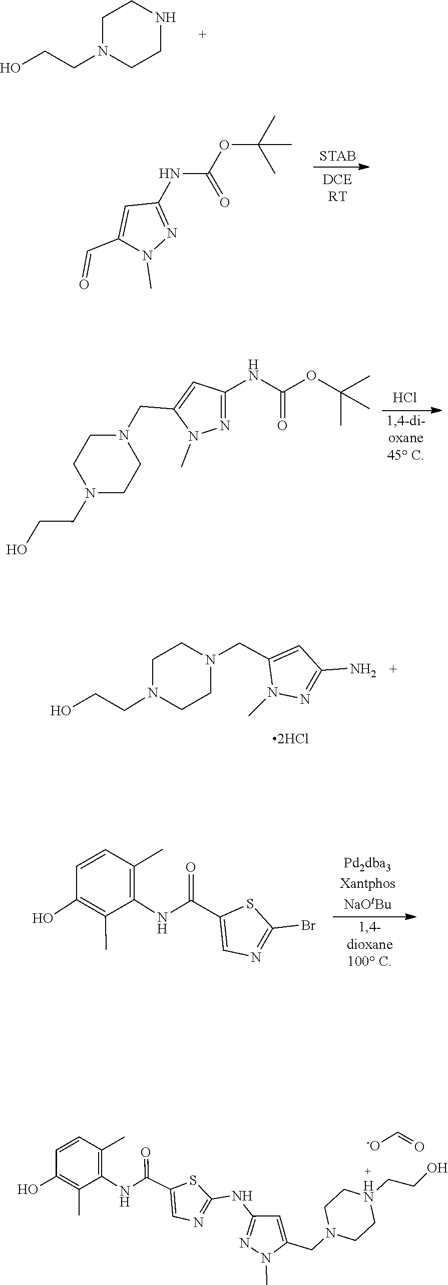

Step 1: To tert-butyl N-(5-formyl-1-methyl-pyrazol-3-yl)carbamate (15 mg, 0.0666 mmol, 1.0 eq), prepared according to International Patent Publication WO 2019/068772 A1 in DCE (2 mL) was added 1-(2-hydroxyethyl) piperazine (0.0090 mL, 0.0733 mmol, 1.1 eq) followed by sodium triacetoxyborohydride (23 mg, 0.107 mmol, 1.6 eq) and the reaction stirred at RT for 1 h, then at 45° C. for 3 h. The reaction was quenched by the addition of aq. Na₂CO₃, brought to pH 10 with aq. NaOH then extracted with EtOAc. The combined organic phases were washed with aq. NH₄Cl
(10% w/w, 10 mL), brine (20 mL), dried (MgSO₄), filtered and concentrated in vacuo to afford tert-butyl N-[5-[4-(2-hydroxyethyl) piperazin-1-yl]methyl]-1-methyl-pyrazol-3-yl]carbamate (22 mg, 0.0648 mmol, 97%). MS (ES+) m/z 340 (M+H). ¹H NMR (300 MHz, Methanol-d4) δ 6.23 (s, 1H), 3.73 (s, 3H), 3.68 (t, J=6.0 Hz, 2H), 3.51 (s, 2H), 2.56 (t, J=6.0 Hz, 10H), 1.50 (s, 9H).

Step 2: To tert-butyl N-[5-[4-(2-hydroxyethyl) piperazin-1-yl]methyl]-1-methyl-pyrazol-3-yl]carbamate (22 mg, 0.0648 mmol, 1.0 eq) was added hydrochloric acid (4M in 1,4-dioxane, 5.0 mL, 20.0 mmol, 309 eq) and the reaction stirred for 3 h at rt, then 4 h at 45° C. The reaction was concentrated in vacuo to give 2-(4-((3-amino-1-methyl-1H-pyrazol-5-yl)methyl) piperazin-1-yl) ethan-1-ol dihydrochloride (20 mg, 0.0641 mmol, 99%) which was used directly in the next step.

Step 3: Prepared as described in Method C from 2-bromo-N-(3-hydroxy-2,6-dimethyl-phenyl)thiazole-5-carboxamide (60 mg, 0.183 mmol, 1.0 eq), 2-(4-((3-amino-1-methyl-1H-pyrazol-5-yl)methyl) piperazin-1-yl) ethan-1-ol dihydrochloride (57 mg, 0.183 mmol, 1.0 eq), sodium tert-butoxide (97 mg, 1.01 mmol, 5.5 eq), Pd₂dba₃ (17 mg, 0.0183 mmol, 0.1 eq) and XantPhos (11 mg, 0.0183 mmol, 0.1 eq) in 1,4-dioxane (4 mL) to return the title compound (BAA-052)(1.2 mg, 0.00196 mmol, 1.1%) as the mono-formate salt (79-85% purity based on LCMS and ¹H NMR) after normal phase chromatography (SiO₂) eluting with 0-100% EtOAc: PE, 10-80% MeOH(containing 10% w/v ammonium hydroxide): DCM and subsequent preparative HPLC(pH). MS (ES+) m/z 486 (M+H).¹H NMR (300 MHz, Methanol-d4) δ 8.02 (s, 1H), 6.96-6.87 (m, 1H), 6.68 (d, J=8.2 Hz, 1H), 6.02 (s, 1H), 3.83 (s, 3H), 3.71 (t, J=5.9 Hz, 2H), 3.58 (s, 2H), 2.80-2.52 (m, 10H), 2.16 (s, 3H), 2.11 (s, 3H).

### General Method E: N-Boc Deprotection:

To a solution or suspension of N-boc protected intermediate (1.0 eq) in Et₂O/MeOH or 1,4-dioxane (0.02-0.1 M) was added HCl (2M Et₂O or 4M in 1,4-dioxane, 10-233 eq) dropwise at 0° C. or RT then and the reaction was stirred at RT for 2-18 h. Reaction monitoring by consumption of starting material via UPLC. Additional HCl (4M in 1,4-dioxane) added if required. The mixture was then concentrated in vacuo, the residue triturated with acetone or DCM/Et₂O, filtered, washed with Et₂O or acetone and dried at 50° C. in vacuo. No further purification was necessary.

### 2-[[1-(2-Aminoethyl)pyrazol-3-yl]amino]-N-(3-hydroxy-2,6-dimethyl-phenyl)thiazole-5-carboxamide (BAA-053)

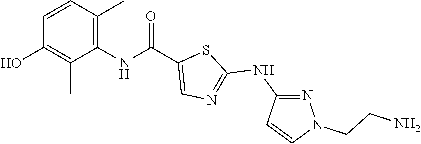 [SMILES: Cc1ccc(O)c(C)c1NC(=O)c1cnc(Nc2ccn(CCN)n2)s1]

To a suspension of tert-butyl N-[2-[3-[5-[(3-hydroxy-2,6-dimethyl-phenyl) carbamoyl]thiazol-2-yl]amino]pyrazol-1-yl]ethyl]carbamate (BAA-020)(10 mg, 0.0212 mmol, 1.0 eq) in Et₂O (5 mL) was added HCl (2M in Et₂O, 0.11 mL, 0.212 mmol, 10 eq). The mixture was stirred for 18 h at RT. The mixture was concentrated in vacuo and the residue was washed with acetone (3×5 mL). The resulting precipitate was dried in vacuo at 50° C. to afford the title compound (BAA-053)(HCl salt; 5.0 mg, 0.0122 mmol, 58%) as a white solid. MS (ES+) m/z 372.9 (M+H). ¹H NMR (300 MHz, Methanol-d4) δ 8.32 (s, 1H), 7.80 (d, J=2.5 Hz, 1H), 6.94 (d, J=8.3 Hz, 1H), 6.72 (d, J=8.2 Hz, 1H), 6.24 (d, J=2.5 Hz, 1H), 4.51 (t, J=5.8 Hz, 2H), 3.52 (t, J=5.7 Hz, 2H), 2.17 (s, 3H), 2.11 (s, 3H).

The following example compounds were prepared similarly using Method E:

| # | Structure & Name | Analytical Data |
|---|---|---|
| BAA-054 | 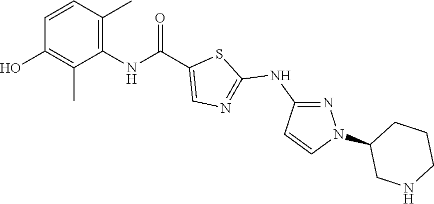 [SMILES: Cc1ccc(O)c(C)c1NC(=O)c1cnc(Nc2ccn([C@H]3CCCNC3)n2)s1] | MS (ES+) m/z 413.4 (M + H). ¹H NMR (300 MHz, Methanol-d₄) δ 7.94 (s, 1H), 7.59 (d, J = 2.1 Hz, 1H), 6.91 (d, J = 8.2 Hz, 1H), 6.69 (d, J = 8.2 Hz, 1H), 6.30 (s, 1H), 3.78-3.55 (m, 5H), 2.16-1.97 (m, 9H), 1.88- 1.77 (m, 1H). |
| | N-(3-hydroxy-2,6-dimethyl-phenyl)- | |
| | 2-[1-[(3S)-3-piperidyl]pyrazol-3- | |
| | yllamino]thiazole-5-carboxamide | |
| | (hydrochloride) | |
| BAA-055 | 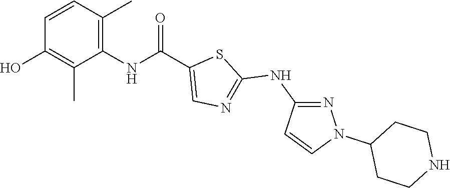 [SMILES: Cc1ccc(O)c(C)c1NC(=O)c1cnc(Nc2ccn(C3CCNCC3)n2)s1] | MS (ES+) m/z 413.0 (M + H). ¹H NMR (300 MHz, DMSO-d₆) δ 9.67 (s, 1H), 9.17 (s, 1H), 8.90 (s, 1H), 8.12 (s, 1H), 7.48 (d, J = 1.8 Hz, 1H), 6.88 (d, J = 8.2 Hz, 1H), 6.71 (d, J = 8.2 Hz, 1H), 6.31 (s, 1H), 4.73-4.53 (m, 1H), 4.04 (br s, 2H), 3.40 (d, J = 11.9 Hz, 2H), 3.04 (q, J = 11.1 Hz, 2H), 2.39-2.10 (m, 2H), 2.01 (d, J = 24.8 Hz, 8H). |
| | N-(3-hydroxy-2,6-dimethyl-phenyl)- | |
| | 2-[[1-(4-piperidyl)pyrazol-3- | |
| | yllamino]thiazole-5-carboxamide | |
| | (hydrochloride) | |
| BAA-056 | 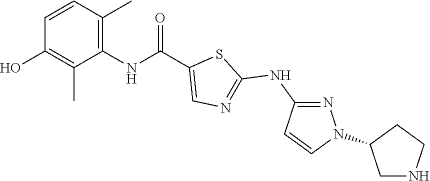 [SMILES: Cc1ccc(O)c(C)c1NC(=O)c1cnc(Nc2ccn([C@@H]3CCNC3)n2)s1] | MS (ES+) m/z 399.3 (M + H). ¹H NMR (DMSO-d₆, 300 MHz) δ 9.77 (br s, 1H), 9.71 (s, 1H), 9.32 (br s, 1H), 8.16 (s, 1H), 7.53 (d, 1H, J = 2.0 Hz), 6.87 (d, 1H, J = 8.2 Hz), 6.69 (d, 1H, J = 8.2 Hz), 6.27 (s, 1H), 5.26 (tt, 1H, J = 7.4, 4.1 Hz), 3.65-3.58 (m, 1H), 3.50-3.30 (m, 4H), 2.41-2.28 (m, 1H), 2.19-2.10 (m, 1H), 2.05 (s, 3H), 1.96 (s, 3H) ppm. |
| | N-(3-hydroxy-2,6-dimethyl-phenyl)- | |
| | 2-[[1-[(3R)-pyrrolidin-3-yl]pyrazol-3- | |
| | yllamino]thiazole-5-carboxamide | |
| | (hydrochloride) | |
| BAA-057 | 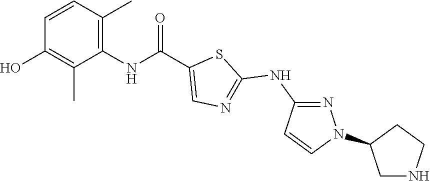 [SMILES: Cc1ccc(O)c(C)c1NC(=O)c1cnc(Nc2ccn([C@H]3CCNC3)n2)s1] | MS (ES+) m/z 399.4 (M + H). ¹H NMR (300 MHz, DMSO-d₆) δ 9.73- 9.05 (m, 3H), 8.12 (s, 1H), 7.54 (d, J = 1.9 Hz, 1H), 6.89 (d, J = 8.2 Hz, 1H), 6.70 (d, J = 8.2 Hz, 1H), 6.28 (s, 1H), 5.27 (dt, J = 10.9, 5.3 Hz, 1H), 3.65- 3.27 (m, 5H), 2.44-2.09 (m, 2H), 2.06 (s, 3H), 1.98 (s, 3H). |
| | N-(3-hydroxy-2, 6-dimethyl-phenyl)- | |
| | 2-[1-[(3S)-pyrrolidin-3-yl]pyrazol-3- | |
| | yllamino]thiazole-5-carboxamide | |
| | (hydrochloride) | |
| BAA-260 | 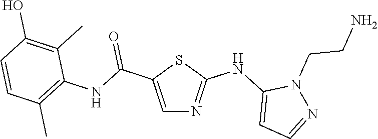 [SMILES: Cc1ccc(O)c(C)c1NC(=O)c1cnc(Nc2ccnn2CCN)s1] | ¹H NMR (300 MHz, MeOD) δ 7.97 (s, 1H), 7.69 (d, J = 2.2 Hz, 1H), 6.92 (d, J = 8.3 Hz, 1H), 6.69 (d, J = 8.3 Hz, 1H), 6.36 (d, J = 2.3 Hz, 1H), 4.44 (t, J = 5.7 Hz, 2H), 3.44 (t, J = 5.6 Hz, 2H), 2.14 (s, 3H), 2.09 (s, 3H). MS: (ES+) m/z 373.3 (M + H)⁺. |
| | 2-((1-(2-aminoethyl)-1H-pyrazol-5- | |
| | yl)amino)-N-(3-hydroxy-2,6- | |
| | dimethylphenyl)thiazole-5- | |
| | carboxamide | |
| BAA-274 | 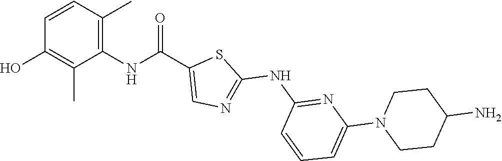 [SMILES: Cc1ccc(O)c(C)c1NC(=O)c1cnc(Nc2cccc(N3CCC(N)CC3)n2)s1] | ¹H NMR (300 MHz, Methanol- d4/chloroform-d) δ 8.43 (s, 1H), 7.72 (t, J = 7.9 Hz, 1H), 6.94 (d, J = 8.2 Hz, 1H), 6.80-6.65 (m, 2H), 6.60 (d, J = 7.6 Hz, 1H), 4.53-4.42 (m, 2H), 3.46 (m, 1H), 3.26-3.12 (m, 2H), 2.20- 2.08 (m, 8H), 1.76-1.65 (m, 2H). MS (ES+) m/z 439.2 (M + H)⁺ |
| | 2-((6-(4-aminopiperidin-1-yl)pyridin- | |
| | 2-yl)amino)-N-(3-hydroxy-2,6- | |
| | dimethylphenyl)thiazole-5- | |
| | carboxamide | |
| BAA-273 | 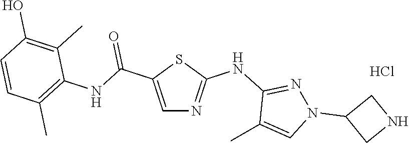 [SMILES: Cc1cn(C2CNC2)nc1Nc1ncc(C(=O)Nc2c(C)ccc(O)c2C)s1 ; Cl] | ¹H NMR (300 MHz, MeOD) δ 8.35 (s, 1H), 7.58 (s, 1H), 6.96 (d, J = 8.2 Hz, 1H), 6.74 (d, J = 8.2 Hz, 1H), 5.46- 5.38 (m, 1H), 4.76-4.65 (m, 2H), 4.59-4.46 (m, 2H), 2.19 (s, 3H), 2.15 (s, 3H), 2.14 (s, 3H); MS (ES+) m/z 399.1 (M + H)⁺ |
| | 2-((1-(azetidin-3-yl)-4-methyl-1H- | |
| | pyrazol-3-yl)amino)-N-(3-hydroxy- | |
| | 2,6-dimethylphenyl)thiazole-5- | |
| | carboxamide hydrochloride | |

### N-(3-Hydroxy-2,6-dimethylphenyl)-2-((6-(piperazin-1-yl)pyrimidin-4-yl)amino)thiazole-5-carboxamide( ) BAA-575

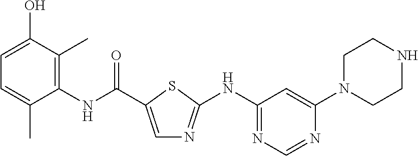 [SMILES: Cc1ccc(O)c(C)c1NC(=O)c1cnc(Nc2cc(N3CCNCC3)ncn2)s1]

To a solution of tert-butyl 4-(6-aminopyrimidin-4-yl) piperazine-1-carboxylate (138 mg, 0.494 mmol) and 2-bromo-N-[3-[tert-butyl(dimethyl) silyl]oxy-2,6-dimethyl-phenyl]thiazole-5-carboxamide
(104 mg, 0.235 mmol) in 1,4-Dioxane (2.35 mL) was added palladium (II)acetate (5.3 mg, 0.0235 mmol), 4,5-bis(diphenylphosphino)-9,9-dimethylxanthene (Xantphos)(27 mg, 0.0470 mmol) and potassium carbonate 325 mesh (88 mg, 0.635 mmol) then the reaction was sparged with nitrogen for 5 mins, brought to 80° C. and stirred for 16 h. 4 M Hydrogen chloride solution in 1-4, dioxane 10
(0.59 mL, 2.35 mmol) and water (1 mL) were added and the reaction mixture was stirred for further 48 h. The mixture was precipitated in water, filtered, washed with i-hexane and purified by prep HPLC to provide the title compound (3.2 mg, 0.00699 mmol, 2.98% yield) as a white solid. MS (ES+) m/z=426.2 [M+H]+. ¹H NMR (400 MHz, METHANOL-D4) δ 8.54-8.43 (m, 3H), 8.13 (s, 1H), 6.95-6.89 (m, 1H), 6.69 (d, J=8.2 Hz, 1H), 6.29 (d, J=1.1 Hz, 1H), 3.87 (t, J=5.3 Hz, 4H), 3.29-3.22 (m, 4H), 2.17 (d, J=0.7 Hz, 3H), 2.12 (s, 3H).

### Synthesis of Carboxylic Acids by Ester Hydrolysis

### 4-[3-[[5-[(3-Hydroxy-2,6-dimethyl-phenyl) carbamoyl]thiazol-2-yl]amino]pyrazol-1-yl]butanoic acid (BAA-058)

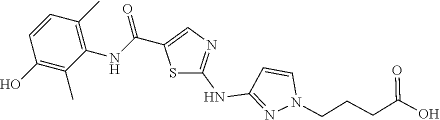 [SMILES: Cc1ccc(O)c(C)c1NC(=O)c1cnc(Nc2ccn(CCCC(=O)O)n2)s1]

To a solution of aq. NaOH(2M in water, 0.11 mL, 0.211 mmol, 7.2 eq) in EtOH(0.2 mL) was added ethyl 4-[3-[5-[(3-hydroxy-2,6-dimethyl-phenyl) carbamoyl]thiazol-2-yl]amino]pyrazol-1-yl]butanoate (13 mg, 0.0293 mmol, 1.0 eq) at RT. The mixture was stirred for 2 h then concentrated in vacuo. The mixture was diluted with water (1 mL) and acidified to pH 4 with aq. HCl (2M). The crude product was extracted with EtOAc (2×1 mL), dried (MgSO₄), filtered and chromatographed (C18) eluting with 0-100% MeCN: H₂O to afford the title compound (3.4 mg, 0.00818 mmol, 28%) as a white solid. MS (ES+) m/z 416.3 (M+H). ¹H NMR (300 MHz, Methanol-d4) δ 8.05 (s, 1H), 7.54 (d, J=2.3 Hz, 1H), 7.00-6.87 (m, 1H), 6.70 (d, J=8.2 Hz, 1H), 6.04 (d, J=2.3 Hz, 1H), 4.17 (t, J=6.6 Hz, 2H), 2.40-2.26 (m, 2H), 2.24-2.16 (m, 5H), 2.14 (s, 3H).

### 2-[3-[[5-[(3-Hydroxy-2,6-dimethyl-phenyl) carbamoyl]thiazol-2-yl]amino]pyrazol-1-yl]acetic acid (BAA-059)

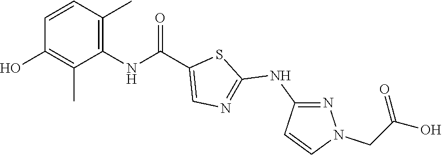 [SMILES: Cc1ccc(O)c(C)c1NC(=O)c1cnc(Nc2ccn(CC(=O)O)n2)s1]

To a stirred solution of ethyl 2-[3-[5-[(3-hydroxy-2,6-dimethyl-phenyl) carbamoyl]thiazol-2-yl]amino]pyrazol-1-yl]acetate (BAA-023)(100 mg, 0.241 mmol, 1.0 eq) in EtOH(2.5 mL) was added aq. NaOH(2M, 2.5 mL, 5.00 mmol, 21 eq) dropwise. The mixture was stirred at RT for 3 h then concentrated in vacuo. The mixture was dissolved in water (10 mL) and adjusted to pH 4 with aq. HCl (2M). The mixture was extracted with EtOAc (2×20 mL), the organics combined, washed with brine (20 mL), filtered through a phase separator and the solvent removed in vacuo to afford the title compound (78 mg, 0.202 mmol, 84%) as a beige solid. MS (ES+) m/z 388.3 (M+H). 1H NMR (300 MHz, Methanol-d4) δ 8.03 (s, 1H), 7.58 (d, J=2.4 Hz, 1H), 6.91 (d, J=8.2 Hz, 1H), 6.68 (d, J=8.2 Hz, 1H), 6.09 (d, J=2.4 Hz, 1H), 4.89 (s, 2H), 2.16 (s, 3H), 2.11 (s, 3H).

### Alternative Route to BAA-043

### tert-Butyl(3R)-3-[3-[[5-[(3-hydroxy-2,6-dimethyl-phenyl) carbamoyl]thiazol-2-yl]amino]pyrazol-1-yl]pyrrolidine-1-carboxylate (BAA-043)

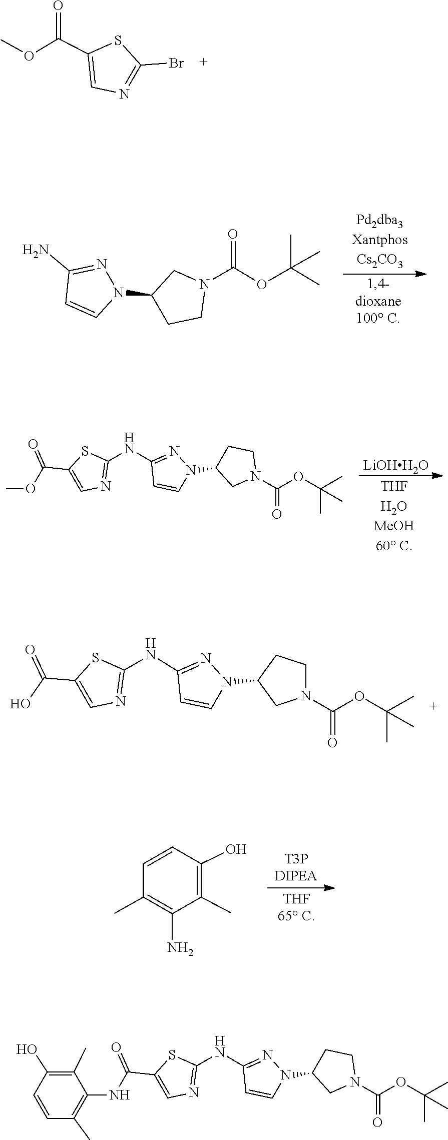

Step 1: Prepared as described in Method D from methyl 2-bromothiazole-5-carboxylate
(200 mg, 0.90 mmol, 1.0 eq), tert-butyl(3R)-3-(3-aminopyrazol-1-yl) pyrrolidine-1-carboxylate (568 mg, 2.25 mmol, 2.5 eq), Pd₂dba₃ (124 mg, 0.135 mmol, 0.15 eq), XantPhos (156 mg, 0.270 mmol, 0.3 eq) and Cs₂CO₃ (738 mg, 2.25 mmol, 2.5 eq) in 1,4-dioxane: H2O (5:1, 18 mL) at 95° C. for 18 h to afford methyl 2-[1-[(3R)-1-tert-butoxycarbonylpyrrolidin-3-yl]pyrazol-3-yl]amino]thiazole-5-carboxylate (116 mg, 0.295 mmol, 33%) as a yellow solid after normal phase chromatography (SiO₂) eluting with 15-100% EtOAc: PE. MS (ES+) m/z 394.3 (M+H). ¹H NMR (300 MHz, Chloroform-d) δ 9.77 (br s, 1H), 7.77 (s, 1H), 7.54 (d, 1H, J=1.7 Hz), 6.23 (d, 1H, J=2.0 Hz), 4.93 (p, 1H, J=6.9 Hz), 3.82 (s, 3H), 3.78-3.57 (m, 3H), 3.47-3.39 (m, 1H), 2.49-2.38 (m, 1H), 2.25-2.14 (m, 1H), 1.44 (s, 9H) ppm.

Step 2: Lithium hydroxide monohydrate (249 mg, 5.79 mmol, 6 eq) was added to a solution of methyl 2-[1-[(3R)-1-tert-butoxycarbonylpyrrolidin-3-yl]pyrazol-3-yl]amino]thiazole-5-carboxylate
(380 mg, 0.97 mmol) in THF: H2O: MeOH(3:2:0.5, 11.0 mL) and the reaction mixture was heated to 60° C., with stirring overnight. The mixture was cooled to RT, acidified with aq. HCl (1M, to PH~4) then separated between DCM (with few drops of MeOH) and brine and the combined organic extracts were dried (MgSO₄), filtered and concentrated to give the crude product, which was purified using normal phase chromatography (SiO₂) eluting with 0-25% MeOH: DCM to afford 2-[[1-[(3R)-1-tert-butoxycarbonylpyrrolidin-3-yl]pyrazol-3-yl]amino]thiazole-5-carboxylic acid (218 mg, 0.575 mmol, 59%) as a beige solid. MS (ES+) m/z 380.3 (M+H). ¹H NMR (300 MHz, Methanol-d4) δ 7.76 (s, 1H), 7.51 (d, 1H, J=2.0 Hz), 6.26 (d, 1H, J=2.0 Hz), 5.09 (p, 1H, J=6.8 Hz), 3.77-3.42 (m, 4H), 2.38-2.28 (m, 2H), 1.47 (d, 9H, J=6.3 Hz) ppm.

Step 3: To a stirring solution of 2-[1-[(3R)-1-tert-butoxycarbonylpyrrolidin-3-yl]pyrazol-3-yl]amino]thiazole-5-carboxylic acid (192 mg, 0.51 mmol, 1.0 eq), 3-amino-2,4-dimethyl-phenol (76 mg, 0.56 mmol, 1.1 eq) and DIPEA (0.22 mL, 1.27 mmol, 2.5 eq) in anhydrous THF (5 mL) was added T3P (50% in EtOAc, 0.42 mL, 0.71 mmol, 1.4 eq) dropwise. The reaction mixture was heated to 65° C. and left to stir overnight. The mixture was allowed to cool to RT, water and EtOAc added, and the layers separated. The organics were washed with brine, dried (MgSO₄), filtered and concentrated in vacuo. The crude material was chromatographed (SiO₂) eluting with 60-100% EtOAc: PE to afford the title compound (115 mg, 46% yield) as a yellow solid. MS (ES+) m/z 499.0 (M+H).

### Nucleophilic Substitution of 2-bromo-N-(3-hydroxy-2.6-dimethyl-phenyl)thiazole-5-carboxamide with amines

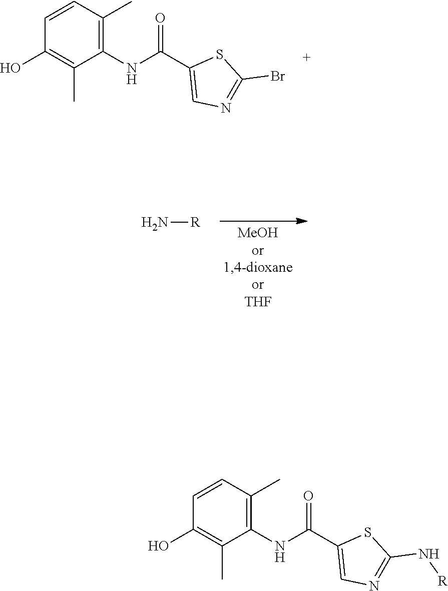

General Method F: Nucleophilic Substitution of 2-Bromo-N-(3-Hydroxy-2,6-Dimethyl-Phenyl)Thiazole-5-Carboxamide with Amines

To a solution of 2-bromo-N-(3-hydroxy-2,6-dimethyl-phenyl)thiazole-5-carboxamide (1 eq) in MeOH, THF or 1,4-dioxane (0.02-0.07M) was added amine (2-10 eq). DIPEA (2 eq) added if amine was hydrochloride salt. The reaction mixture was stirred for 18 h-2 days at RT, 50° C. or 100° C. The reaction was monitored by consumption of starting material via UPLC. When required, additional amine was added to push reaction to completion. The mixture was concentrated in vacuo or partitioned between Et₂O and water, the organics washed with sat. aq. NH₄Cl and concentrated in vacuo. The crude material was purified by either normal phase chromatography (SiO₂) using a gradient of MeOH: DCM (optionally containing 1% aq. NH₃), reverse phase chromatography (C18) using a gradient of MeCN: H₂O and/or by preparative HPLC-MS using a gradient of high or low pH aq. MeCN.

### N-(3-Hydroxy-2,6-dimethyl-phenyl)-2-(methylamino)thiazole-5-carboxamide (BAA-221)

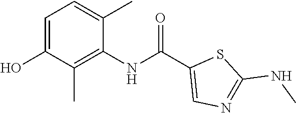 [SMILES: CNc1ncc(C(=O)Nc2c(C)ccc(O)c2C)s1]

Prepared as described in Method F from 2-bromo-N-(3-hydroxy-2,6-dimethyl-phenyl)thiazole-5-carboxamide (168 mg, 0.513 mmol, 1.0 eq) and methylamine (2M in THF, 2.6 mL, 5.13 mmol, 10.0 eq) in MeOH(25 mL) at RT to afford the title compound (47 mg, 0.169 mmol, 33%) as a beige powder after normal phase chromatography (SiO₂) using 0-20% MeOH: DCM as eluent. MS (ES+) m/z 278.2 (M+H). ¹H NMR (300 MHz, DMSO-d₆) δ 9.35 (s, 1H), 9.12 (s, 1H), 8.07 (q, J=4.7 Hz, 1H), 7.92 (s, 1H), 6.86 (d, J=8.2 Hz, 1H), 6.66 (d, J=8.2 Hz, 1H), 2.84 (d, J=4.7 Hz, 3H), 2.04 (s, 3H), 1.96 (s, 3H).

The following example compounds were prepared similarly using Method F using the appropriately substituted amine.

| # | Structure & Name | Amine | Analytical Data |
|---|---|---|---|
| BAA- 060 | 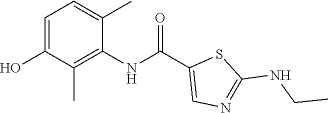 [SMILES: CCNc1ncc(C(=O)Nc2c(C)ccc(O)c2C)s1] | 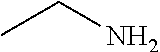 | MS (ES+) m/z 292.1 (M + H). ¹H NMR (300 MHz, Methanol-d₄) δ 7.87 (s, 1H), 6.90 (d, J = 8.2 Hz, 1H), 6.67 (d, J = 8.2 Hz, 1H), 3.44-3.33 (m, 2H), |
| | | | 2.14 (s, 3H), 2.09 (s, |
| | 2-(ethylamino)-N-(3-hydroxy-2,6-dimethyl- | | 3H), 1.27 (t, J = 7.2 Hz, |
| | phenyl)thiazole-5-carboxamide | | 3H). |
| BAA- 061 | 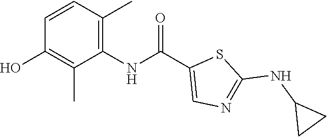 [SMILES: Cc1ccc(O)c(C)c1NC(=O)c1cnc(NC2CC2)s1] | 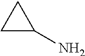 | MS (ES+) m/z 304.1 (M + H). ¹H NMR (300 MHz, Methanol-d₄) δ 7.92 (s, 1H), 6.90 (d, J = 8.2 Hz, 1H), 6.68 (d, J = 8.2 Hz, 1H), 2.63 (tt, J = 6.8, 3.6 Hz, 1H), 2.15 (s, 3H), 2.10 (s, 3H), 0.85 (td, J = |
| | | | 7.2, 5.0 Hz, 2H), 0.68- |
| | 2-(cyclopropylamino)-N-(3-hydroxy-2,6- | | 0.63 (m, 2H). |
| | dimethyl-phenyl)thiazole-5-carboxamide | | |
| BAA- 062 | 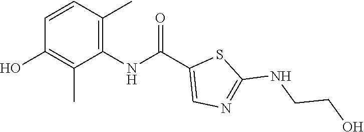 [SMILES: Cc1ccc(O)c(C)c1NC(=O)c1cnc(NCCO)s1] | 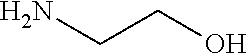 | MS (ES+) m/z 308.3 (M + H). ¹H NMR (300 MHz, DMSO-d₆) δ 9.35 (s, 1H), 9.13 (s, 1H), 8.16 (t, J = 5.6 Hz, 1H), 7.90 (s, 1H), 6.87 (d, J = 8.2 Hz, 1H), 6.67 (d, J = 8.2 Hz, 1H), |
| | | | 4.79 (t, J = 5.4 Hz, 1H), |
| | N-(3-hydroxy-2,6-dimethyl-phenyl)-2-(2- | | 3.56 (q, J = 5.7 Hz, 2H), |
| | hydroxyethylamino)thiazole-5-carboxamide | | 3.39-3.27 (m, 1H), 3.18 |
| | | | (d, J = 5.2 Hz, 1H), 2.05 |
| | | | (s, 3H), 1.97 (s, 3H). |
| BAA- 063 | 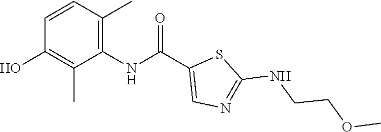 [SMILES: COCCNc1ncc(C(=O)Nc2c(C)ccc(O)c2C)s1] | 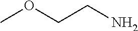 | MS (ES+) m/z 322.0 (M + H). ¹H NMR (300 MHz, Methanol-d₄) δ 7.86 (s, 1H), 6.90 (d, J = 8.2 Hz, 1H), 6.67 (d, J = 8.2 Hz, 1H), 3.60 (dd, J = 7.9, 3.1 Hz, 2H), 3.52 (dd, J = |
| | | | 7.9, 3.1 Hz, 2H), 3.39 (s, |
| | N-(3-hydroxy-2,6-dimethyl-phenyl)-2-(2- | | 3H), 2.14 (s, 3H), 2.09 |
| | methoxyethylamino)thiazole-5- | | (s, 3H). |
| | carboxamide | | |
| BAA- 064 | 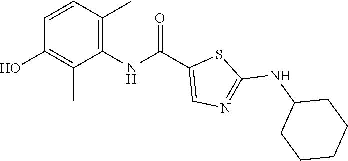 [SMILES: Cc1ccc(O)c(C)c1NC(=O)c1cnc(NC2CCCCC2)s1] | 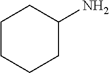 | MS (ES+) m/z 346.3 (M + H). ¹H NMR (300 MHz, DMSO-d₆) δ 9.33 (s, 1H), 9.13 (s, 1H), 8.10 (d, J = 7.6 Hz, 1H), 7.88 (s, 1H), 6.87 (d, J = 8.2 Hz, 1H), 6.67 (d, J = 8.2 Hz, 1H), 3.50 (s, 1H), 2.05 (s, 3H), 1.97 (s, 5H), 1.72 |
| | | | (d, J = 14.8 Hz, 2H), 1.58 |
| | 2-(cyclohexylamino)-N-(3-hydroxy-2,6- | | (d, J = 12.2 Hz, 1H), 1.41- |
| | dimethyl-phenyl)thiazole-5-carboxamide | | 1.16 (m, 5H). |
| BAA- 065 | 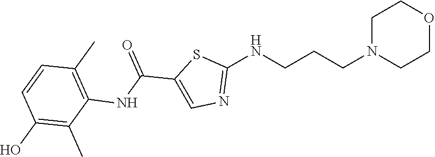 [SMILES: Cc1ccc(O)c(C)c1NC(=O)c1cnc(NCCCN2CCOCC2)s1] | 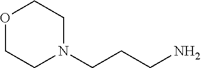 | MS (ES+) m/z 391 (M + H). ¹H NMR (300 MHz, Methanol-d₄) δ 8.26 (s, 1H), 7.79 (s, 1H), 7.50- 7.30 (m, 2H), 6.87 (d, J = 8.3 Hz, 1H), 6.66 (d, J = 8.2 Hz, 1H), 3.74 (t, J = 4.7 Hz, 4H), 3.31 (dq, J = 3.3, 1.5 Hz, 2H), 2.53 (t, |
| | | | J = 6.9 Hz, 6H), 2.12 (s, 3H), 2.08 (s, 3H), 1.85 (p, J = 6.9 Hz, 2H). |
| | N-(3-hydroxy-2,6-dimethyl-phenyl)-2-(3- | | |
| | morpholinopropylamino)thiazole-5- | | |
| | carboxamide | | |
| BAA- 066 | 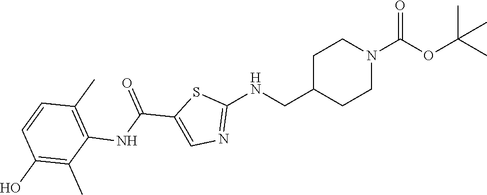 [SMILES: Cc1ccc(O)c(C)c1NC(=O)c1cnc(NCC2CCN(C(=O)OC(C)(C)C)CC2)s1] | 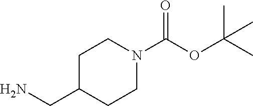 | MS (ES+) m/z 461 (M + H). 1H NMR (300 MHz, Chloroform-d) o 7.82 (d, J = 1.5 Hz, 1H), 7.57 (d, J = 5.4 Hz, 1H), 6.87 (d, J = 8.2 Hz, 1H), 6.66 (d, J = 8.2 Hz, 1H), 4.09 (d, J = 13.5 Hz, 2H), 3.21 (d, J = 6.6 Hz, 2H), 2.84- 2.52 (m, 2H), 2.27-2.02 (m, 9H), 1.76 (d, J = 13.9 |
| | | | Hz, 2H), 1.43 (d, J = 1.4 |
| | tert-butyl 4-[[5-[(3-hydroxy-2,6-dimethyl- | | Hz, 9H), 1.28-0.98 (m, |
| | phenyl)carbamoyl]thiazol-2- | | 2H). |
| | yl]amino]methyl]piperidine-1-carboxylate | | |
| BAA- 067 | 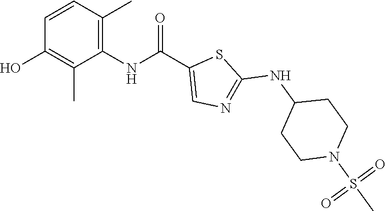 [SMILES: Cc1ccc(O)c(C)c1NC(=O)c1cnc(NC2CCN(S(C)(=O)=O)CC2)s1] | 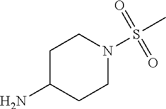 | MS (ES+) m/z 425 (M + H). ¹H NMR (300 MHz, Methanol-d₄) δ 8.54 (s, 1H), 7.88 (s, 1H), 6.90 (d, J = 8.3 Hz, 1H), 6.67 (d, J = 8.2 Hz, 1H), 3.88- 3.61 (m, 2H), 2.97 (t, J = 11.1 Hz, 2H), 2.86 (s, 3H), 2.26-1.99 (m, 8H), 1.78-1.53 (m, 2H). |
| | N-(3-hydroxy-2,6-dimethyl-phenyl)-2-[(1- | | |
| | methylsulfonyl-4-piperidyl)amino]thiazole- | | |
| | 5-carboxamide | | |
| BAA- 068 | 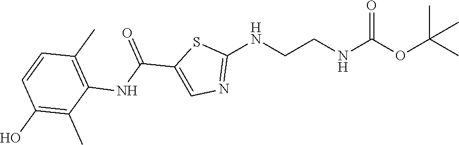 [SMILES: Cc1ccc(O)c(C)c1NC(=O)c1cnc(NCCNC(=O)OC(C)(C)C)s1] | 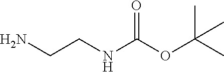 | MS (ES+) m/z 407 (M + H). 1H NMR (300 MHz, Methanol-d4) 0 7.85 (s, 1H), 6.89 (d, J = 8.2 Hz, 1H), 6.67 (d, J = 8.2 Hz, 1H), 3.42 (t, J = 6.0 Hz, 2H), 3.30-3.26 (m, 2H), 2.13 (s, 3H), 2.09 (s, 3H), 1.43 (s, 9H). |
| | tert-butyl N-[2-[5-[(3-hydroxy-2,6-dimethyl- | | |
| | phenyl)carbamoyl]thiazol-2- | | |
| | yllamino]ethyl]carbamate | | |
| BAA- 069 | 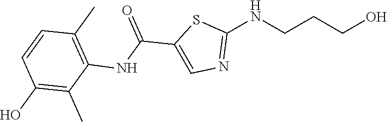 [SMILES: Cc1ccc(O)c(C)c1NC(=O)c1cnc(NCCCO)s1] | 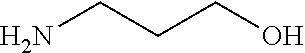 | MS (ES+) m/z 322 (M + H). ¹H NMR (300 MHz, Methanol-d₄) δ 8.40 (br s, 1H), 7.87 (s, 1H), 6.90 (d, J = 8.2 Hz, 1H), 6.68 (d, J = 8.2 Hz, 1H), 3.67 (t, J = 6.2 Hz, 2H), 3.45 |
| | | | (t, J = 6.9 Hz, 2H), 2.15 |
| | N-(3-hydroxy-2,6-dimethyl-phenyl)-2-(3- | | (s, 3H), 2.10 (s, 3H), |
| | hydroxypropylamino)thiazole-5- | | 1.87 (p, J = 6.6 Hz, 2H). |
| | carboxamide | | |
| BAA- 070 | 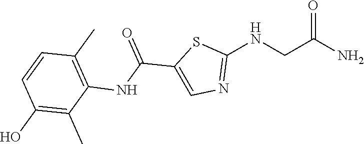 [SMILES: Cc1ccc(O)c(C)c1NC(=O)c1cnc(NCC(N)=O)s1] | 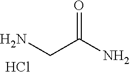 | MS (ES+) m/z 321 (M + H). ¹H NMR (300 MHz, Methanol-d₄) δ 8.54 (s, 1H), 7.88 (s, 1H), 6.90 (d, J = 8.2 Hz, 1H), 6.67 (d, J = 8.2 Hz, 1H), 4.04 (s, 2H), 2.14 (s, 3H), 2.09 (s, 3H). |
| | 2-[(2-amino-2-oxo-ethyl)amino]-N-(3- | | |
| | hydroxy-2,6-dimethyl-phenyl)thiazole-5- | | |
| | carboxamide | | |
| BAA- 071 | 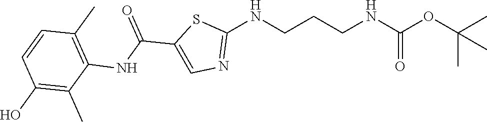 [SMILES: Cc1ccc(O)c(C)c1NC(=O)c1cnc(NCCCNC(=O)OC(C)(C)C)s1] | 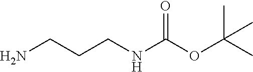 | MS (ES+) m/z 421 (M + H). ¹H NMR (300 MHz, Methanol-d₄) δ 7.87 (s, 1H), 6.90 (dt, J = 8.2, 0.7 Hz, 1H), 6.67 (d, J = 8.2 Hz, 1H), 3.37 (t, J = 6.8 Hz, 2H), 3.15 (t, J = 6.7 |
| | | | Hz, 2H), 2.14 (s, 3H), |
| | tert-butyl N-[3-[5-[(3-hydroxy-2,6-dimethyl- | | 2.09 (s, 3H), 1.81 (p, J = |
| | phenyl)carbamoyl]thiazol-2- | | 6.8 Hz, 2H), 1.44 (s, 9H). |
| | yllamino]propyl]carbamate | | |
| BAA- 072 | 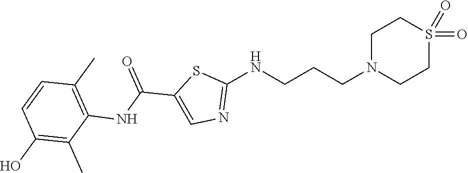 [SMILES: Cc1ccc(O)c(C)c1NC(=O)c1cnc(NCCCN2CCS(=O)(=O)CC2)s1] | 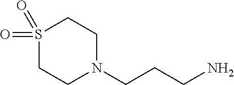 [SMILES: NCCCN1CCS(=O)(=O)CC1] | MS (ES+) m/z 439 (M + H). ¹H NMR (300 MHz, Methanol-d₄) δ 7.88 (s, 1H), 6.90 (dt, J = 8.3, 0.7 Hz, 1H), 6.67 (d, J = 8.2 Hz, 1H), 4.64 (s, 2H), 3.42 (t, J = 6.7 Hz, 2H), 3.12 (dd, J = 7.3, 3.1 Hz, 4H), 3.00 (dd, J = 7.1, 3.3 Hz, 4H), 2.63 (t, J = |
| | | | 6.9 Hz, 2H), 2.18-2.11 |
| | 2-[3-(1, 1-dioxo-1,4-thiazinan-4- | | (m, 3H), 2.09 (s, 3H), |
| | yl)propylamino]-N-(3-hydroxy-2,6-dimethyl- | | 1.85 (p, J = 6.8 Hz, 2H). |
| | phenyl)thiazole-5-carboxamide | | |
| BAA- 073 | 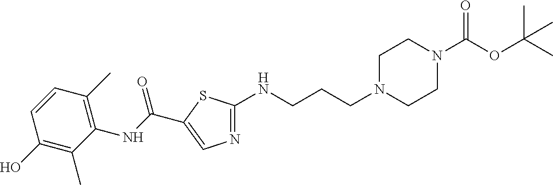 [SMILES: Cc1ccc(O)c(C)c1NC(=O)c1cnc(NCCCN2CCN(C(=O)OC(C)(C)C)CC2)s1] | 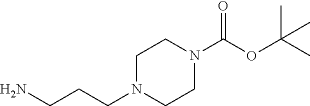 | MS (ES+) m/z 490 (M + H). ¹H NMR (300 MHz, Methanol-d₄) δ 8.41 (s, 1H), 7.88 (s, 1H), 6.90 (d, J = 8.3 Hz, 1H), 6.67 (d, J = 8.2 Hz, 1H), 3.43 (dd, J = 15.5, 8.8 Hz, 6H), 2.56 (dd, J = 13.7, 6.5 Hz, 6H), 2.14 (s, 3H), 2.09 (s, 3H), 1.90 (p, J = |
| | | | 7.0 Hz, 2H), 1.46 (s, 9H). |
| | tert-butyl 4-[3-[[5-[(3-hydroxy-2,6-dimethyl- | | |
| | phenyl)carbamoyl]thiazol-2- | | |
| | yl]amino]propyl]piperazine-1-carboxylate | | |
| BAA- 074 | 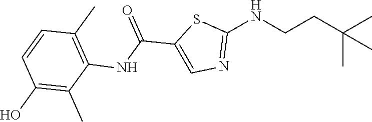 [SMILES: Cc1ccc(O)c(C)c1NC(=O)c1cnc(NCCC(C)(C)C)s1] | 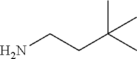 | MS (ES+) m/z 348 (M + H) ¹H NMR (300 MHz, Methanol-d₄) δ 7.87 (s, 1H), 6.94-6.86 (m, 1H), 6.67 (d, J = 8.2 Hz, 1H), 3.42-3.33 (m, 2H), 2.14 (d, J = 0.6 Hz, 3H), 2.09 |
| | | | (s, 3H), 1.69-1.51 (m, |
| | 2-(3,3-dimethylbutylamino)-N-(3-hydroxy- | | 2H), 0.99 (s, 9H). |
| | 2,6-dimethyl-phenyl)thiazole-5- | | |
| | carboxamide | | |
| BAA- 075 | 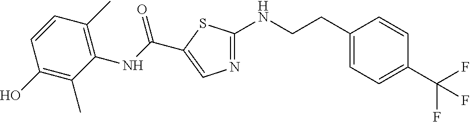 [SMILES: Cc1ccc(O)c(C)c1NC(=O)c1cnc(NCCc2ccc(C(F)(F)F)cc2)s1] | 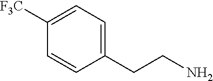 [SMILES: NCCc1ccc(C(F)(F)F)cc1] | MS (ES+) m/z 436 (M + H). ¹H NMR (300 MHz, Methanol-d₄) δ 7.88 (s, 1H), 7.66-7.56 (m, 2H), 7.52-7.43 (m, 2H), 6.90 (dt, J = 8.2, 0.7 Hz, 1H), 6.67 (d, J = 8.2 Hz, 1H), |
| | | | 3.64 (dd, J = 7.5, 6.6 Hz, |
| | N-(3-hydroxy-2,6-dimethyl-phenyl)-2-[2-[4- | | 2H), 3.05 (t, J = 7.1 Hz, |
| | (trifluoromethyl)phenyl]ethylamino]thiazole- | | 2H), 2.15-2.12 (m, 3H), |
| | 5-carboxamide | | 2.09 (s, 3H). |
| BAA- 076 | 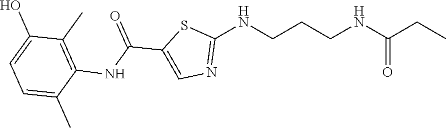 [SMILES: CCC(=O)NCCCNc1ncc(C(=O)Nc2c(C)ccc(O)c2C)s1] | 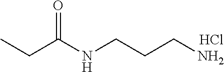 | MS (ES+) m/z 377 (M + H). ¹H NMR (300 MHz, Methanol-d₄) δ 8.04 (s, 1H), 7.87 (s, 1H), 6.90 (dt, J = 8.3, 0.7 Hz, 1H), 6.67 (d, J = 8.2 Hz, 1H), 3.44-3.23 (m, 4H), 2.28- |
| | | | 2.18 (m, 2H), 2.15- |
| | N-(3-hydroxy-2,6-dimethyl-phenyl)-2-[3- | | 2.12 (m, 3H), 2.09 (s, |
| | (propanoylamino)propylamino]thiazole-5- | | 3H), 1.84 (p, J = 6.8 Hz, |
| | carboxamide | | 2H), 1.14 (t, J = 7.6 Hz, |
| | | | 3H). |
| BAA- 077 | 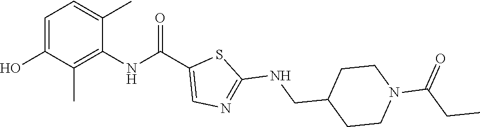 [SMILES: CCC(=O)N1CCC(CNc2ncc(C(=O)Nc3c(C)ccc(O)c3C)s2)CC1] | 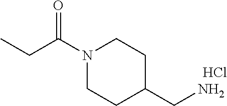 | MS (ES+) m/z 417 (M + H) 1H NMR (300 MHz, Methanol-d4) 0 7.87 (s, 1H), 6.90 (d, J = 8.2 Hz, 1H), 6.68 (d, J = 8.2 Hz, 1H), 3.37-3.23 (m, 4H), 2.65 (d, J = 9.2 Hz, 2H), |
| | | | 2.42 (q, J = 7.5 Hz, 2H), |
| | N-(3-hydroxy-2,6-dimethyl-phenyl)-2-[(1- | | 2.14 (s, 3H), 2.09 (s, |
| | propanoyl-4- | | 3H), 2.07-1.76 (m, 2H), |
| | piperidyl) methylamino]thiazole-5- | | 1.33-1.15 (m, 3H), 1.11 |
| | carboxamide | | (t, J = 7.5 Hz, 3H). |
| BAA- 078 | 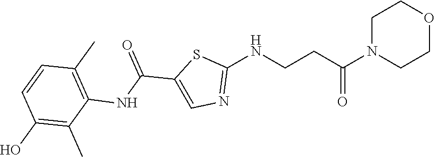 [SMILES: Cc1ccc(O)c(C)c1NC(=O)c1cnc(NCCC(=O)N2CCOCC2)s1] | 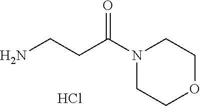 [SMILES: NCCC(=O)N1CCOCC1 ; Cl] | MS (ES+) m/z 405 (M + H) ¹H NMR (300 MHz, Methanol-d4) δ 7.87 (s, 1H), 6.90 (dt, J = 8.2, 0.7 Hz, 1H), 6.67 (d, J = 8.2 Hz, 1H), 3.70-3.62 (m, 6H), 3.62-3.50 (m, 4H), 2.76 (t, J = 6.5 Hz, 2H), 2.14 (d, J = 0.6 Hz, 3H), |
| | | | 2.09 (s, 3H). |
| | N-(3-hydroxy-2,6-dimethyl-phenyl)-2-[(3- | | |
| | morpholino-3-oxo-propyl)amino]thiazole-5- | | |
| | carboxamide | | |

Additional example compounds were prepared similarly using Method F with the appropriate amine.

| # | Structure & Name | Analytical Data |
|---|---|---|
| BAA-344 | 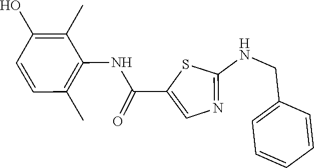 [SMILES: Cc1ccc(O)c(C)c1NC(=O)c1cnc(NCc2ccccc2)s1] | MS (ES+) m/z 354 (M + H). ¹H NMR (300 MHz, Methanol-d4) δ 7.88 (s, 1H), 7.45-7.20 (m, 5H), 6.89 (d, J = 8.2 Hz, 1H), 6.67 (d, J = 8.2 Hz, 1H), 4.54 (s, 2H), 2.13 (s, 3H), 2.09 (s, 3H) |
| | 2-(benzylamino)-N-(3-hydroxy-2,6- | |
| | dimethylphenyl) thiazole-5-carboxamide | |
| BAA-345 | 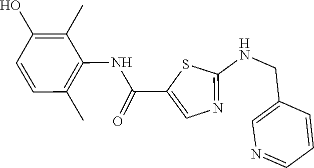 [SMILES: Cc1ccc(O)c(C)c1NC(=O)c1cnc(NCc2cccnc2)s1] | MS (ES+) m/z 355 (M + H). ¹H NMR (300 MHz, Methanol-d4) δ 8.58 (s, 1H), 8.47 (d, J = 4.8 Hz, 1H), 7.92- 7.82 (m, 2H), 7.44 (dd, J = 7.9, 4.9 Hz, 1H), 6.90 (d, J = 8.3 Hz, 1H), 6.67 (d, J = 8.2 Hz, 1H), 4.62 (s, 2H), 2.13 (s, 3H), 2.08 (s, 3H). |
| | N-(3-hydroxy-2,6-dimethylphenyl)-2- | |
| | ((pyridin-3-ylmethyl)amino)thiazole-5- | |
| | carboxamide | |
| BAA-346 | 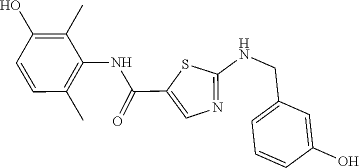 [SMILES: Cc1ccc(O)c(C)c1NC(=O)c1cnc(NCc2cccc(O)c2)s1] | MS (ES+) m/z 370 (M + H). ¹H NMR (300 MHz, Methanol-d4) δ 7.87 (s, 1H), 7.16 (ddd, J = 8.0, 7.3, 0.7 Hz, 1H), 6.89 (dt, J = 8.3, 0.8 Hz, 1H), 6.87-6.79 (m, 2H), 6.73- 6.64 (m, 2H), 4.47 (s, 2H), 2.13 (s, 3H), 2.09 (s, 3H). |
| | N-(3-hydroxy-2,6-dimethylphenyl)-2-((3- | |
| | hydroxybenzyl)amino)thiazole-5- | |
| | carboxamide | |
| BAA-347 | 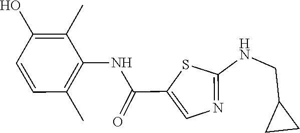 [SMILES: Cc1ccc(O)c(C)c1NC(=O)c1cnc(NCC2CC2)s1] | MS (ES+) m/z 318 (M + H). ¹H NMR (300 MHz, Methanol-d4) δ 7.86 (s, 1H), 6.90 (d, J = 8.2 Hz, 1H), 6.67 (d, J = 8.2 Hz, 1H), 3.21 (d, J = 7.0 Hz, 2H), 2.14 (d, J = 0.6 Hz, 3H), 2.09 (s, 3H), 1.20-1.07 (m, 1H), 0.62-0.54 (m, 2H), 0.29 (dt, J = 6.2, 4.6 Hz, 2H). |
| | 2-(cyclopropylamino)-N-(3-hydroxy-2,6- | |
| | dimethyl-phenyl)thiazole-5-carboxamide | |
| BAA-348 | 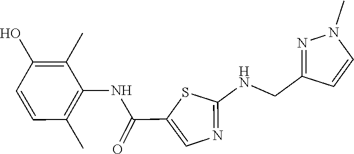 [SMILES: Cc1ccc(O)c(C)c1NC(=O)c1cnc(NCc2ccn(C)n2)s1] | MS (ES+) m/z 358 (M + H). ¹H NMR (300 MHz, Methanol-d4) δ 7.88 (s, 1H), 7.52 (d, J = 2.3 Hz, 1H), 6.94- 6.86 (m, 1H), 6.67 (d, J = 8.2 Hz, 1H), 6.27 (d, J = 2.3 Hz, 1H), 4.51 (s, 2H), 3.86 (s, 3H), 2.15-2.12 (m, 3H), 2.09 (s, 3H). |
| | N-(3-hydroxy-2,6-dimethylphenyl)-2-(((1- | |
| | methyl-1H-pyrazol-3- | |
| | yl)methyl)amino) thiazole-5-carboxamide | |
| BAA-349 | 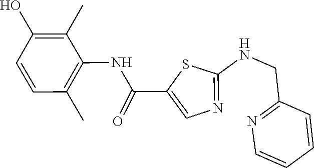 [SMILES: Cc1ccc(O)c(C)c1NC(=O)c1cnc(NCc2ccccn2)s1] | MS (ES+) m/z 355 (M + H). ¹H NMR (300 MHz, Methanol-d4) δ 8.52 (ddd, J = 5.0, 1.8, 0.9 Hz, 1H), 7.90-7.78 (m, 2H), 7.47 (dt, J = 7.9, 1.0 Hz, 1H), 7.33 (ddd, J = 7.6, 4.9, 1.2 Hz, 1H), 6.89 (dt, J = 8.2, 0.7 Hz, 1H), 6.67 (d, J = 8.3 Hz, 1H), 4.68 (s, 2H), 2.13 (s, 3H), 2.08 (s, 3H). |
| | N-(3-hydroxy-2,6-dimethylphenyl)-2- | |
| | ((pyridin-2-ylmethyl)amino) thiazole-5- | |
| | carboxamide | |
| BAA-350 | 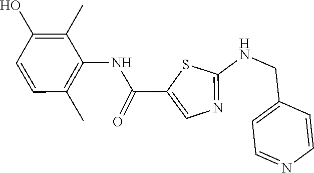 [SMILES: Cc1ccc(O)c(C)c1NC(=O)c1cnc(NCc2ccncc2)s1] | MS (ES+) m/z 355 (M + H). ¹H NMR (300 MHz, Methanol-d4) δ 8.50 (d, J = 5.1 Hz, 2H), 7.86 (s, 1H), 7.49-7.40 (m, 2H), 6.93-6.85 (m, 1H), 6.67 (d, J = 8.2 Hz, 1H), 4.65 (s, 2H), 2.13 (s, 3H), 2.08 (s, 3H). |
| | N-(3-hydroxy-2,6-dimethylphenyl)-2- | |
| | ((pyridin-4-ylmethyl)amino) thiazole-5- | |
| | carboxamide | |
| BAA-351 | 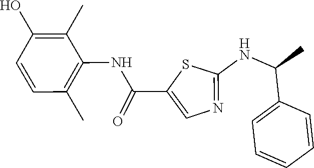 [SMILES: Cc1ccc(O)c(C)c1NC(=O)c1cnc(N[C@@H](C)c2ccccc2)s1] | MS (ES+) m/z 368 (M + H). ¹H NMR (300 MHz, Methanol-d4) δ 7.84 (s, 1H), 7.46-7.20 (m, 5H), 6.95- 6.85 (m, 1H), 6.68 (d, J = 8.2 Hz, 1H), 4.81 (t, J = 7.0 Hz, 1H), 2.91- 2.83 (m, 1H), 2.13 (d, J = 0.6 Hz, 3H), 2.08 (s, 3H), 1.58 (d, J = 6.9 Hz, 3H). |
| | (S)-N-(3-hydroxy-2,6-dimethylphenyl)-2- | |
| | ((1-phenylethyl) amino) thiazole-5- | |
| | carboxamide | |
| BAA-352 | 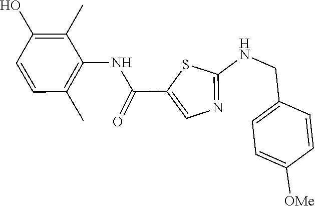 [SMILES: COc1ccc(CNc2ncc(C(=O)Nc3c(C)ccc(O)c3C)s2)cc1] | MS (ES+) m/z 384 (M + H). ¹H NMR (300 MHz, Methanol-d4) δ 7.87 (s, 1H), 7.35-7.26 (m, 2H), 6.95- 6.84 (m, 3H), 6.67 (d, J = 8.2 Hz, 1H), 4.46 (s, 2H), 3.78 (s, 3H), 2.13 (s, 3H), 2.09 (s, 3H). |
| | N-(3-hydroxy-2,6-dimethylphenyl)-2-((4- | |
| | methoxybenzyl)amino)thiazole-5- | |
| | carboxamide | |
| BAA-353 | 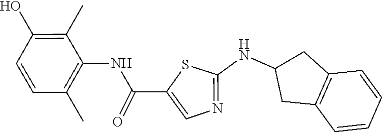 [SMILES: Cc1ccc(O)c(C)c1NC(=O)c1cnc(NC2Cc3ccccc3C2)s1] | MS (ES+) m/z 380 (M + H). ¹H NMR (300 MHz, Methanol-d4) δ 7.90 (s, 1H), 7.29-7.19 (m, 2H), 7.20- 7.08 (m, 2H), 6.90 (d, J = 8.3 Hz, 1H), 6.68 (d, J = 8.2 Hz, 1H), 4.62- 4.47 (m, 2H), 3.45-3.36 (m, 2H), 2.96 (dd, J = 15.9, 5.1 Hz, 2H), 2.15 (d, J = 2.5 Hz, 3H), 2.10 (s, 3H), 0.97-0.78 (m, 1H). |
| | 2-((2,3-dihydro-1H-inden-2-yl)amino)-N- | |
| | (3-hydroxy-2,6-dimethylphenyl)thiazole-5- | |
| | carboxamide | |
| BAA-354 | 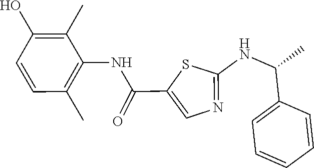 [SMILES: Cc1ccc(O)c(C)c1NC(=O)c1cnc(N[C@H](C)c2ccccc2)s1] | MS (ES+) m/z 368 (M + H). ¹H NMR (300 MHz, Methanol-d4) δ 7.82 (s, 1H), 7.42-7.29 (m, 4H), 7.29- 7.20 (m, 1H), 6.88 (d, J = 8.2 Hz, 1H), 6.66 (d, J = 8.2 Hz, 1H), 4.90- 4.80 (m, 1H), 2.11 (s, 3H), 2.06 (s, 3H), 1.56 (d, J = 6.9 Hz, 3H). |
| | (R)-N-(3-hydroxy-2,6-dimethylphenyl)-2- | |
| | ((1-phenylethyl) amino) thiazole-5- | |
| | carboxamide | |
| BAA-355 | 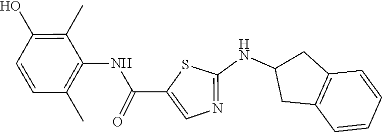 | MS (ES+) m/z 384 (M + H). ¹H NMR (300 MHz, Methanol-d4) δ 7.86 (s, 1H), 7.34-7.23 (m, 2H), 6.99 (d, J = 7.9 Hz, 1H), 6.96-6.86 (m, 2H), 6.67 (d, J = 8.2 Hz, 1H), 4.52 (s, 2H), 3.88 (s, 3H), 2.16-2.11 (m, 3H), 2.09 (s, 3H). |
| | N-(3-hydroxy-2,6-dimethylphenyl)-2-((2- | |
| | methoxybenzyl)amino) thiazole-5- | |
| | carboxamide | |
| BAA-356 | 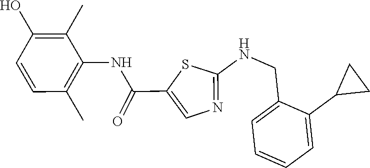 [SMILES: Cc1ccc(O)c(C)c1NC(=O)c1cnc(NCc2ccccc2C2CC2)s1] | MS (ES+) m/z 394 (M + H). ¹H NMR (300 MHz, Methanol-d4) δ 7.89 (s, 1H), 7.33 (dd, J = 7.3, 1.8 Hz, 1H), 7.26-7.12 (m, 2H), 7.11-7.05 (m, 1H), 6.93-6.86 (m, 1H), 6.67 (d, J = 8.2 Hz, 1H), 4.73 (s, 2H), 2.14 (s, 3H), 2.09 (s, 3H), 2.00 (tt, J = 8.5, 5.3 Hz, 1H), 1.03- 0.89 (m, 2H), 0.75-0.62 (m, 2H). |
| | 2-((2-cyclopropylbenzyl)amino)-N-(3- | |
| | hydroxy-2,6-dimethylphenyl)thiazole-5- | |
| | carboxamide | |
| BAA-357 | 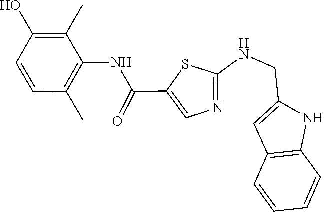 [SMILES: Cc1ccc(O)c(C)c1NC(=O)c1cnc(NCc2cc3ccccc3[nH]2)s1] | MS (ES+) m/z 393 (M + H). ¹H NMR (300 MHz, Methanol-d4) δ 7.91 (s, 1H), 7.47 (dt, J = 7.8, 1.1 Hz, 1H), 7.33 (dq, J = 8.1, 1.0 Hz, 1H), 7.06 (ddd, J = 8.1, 7.0, 1.3 Hz, 1H), 6.97 (ddd, J = 8.1, 7.1, 1.1 Hz, 1H), 6.90 (d, J = 8.3 Hz, 1H), 6.67 (d, J = 8.2 Hz, 1H), 6.41 (d, J = 1.0 Hz, 1H), 4.69 (d, J = 0.9 Hz, 2H), 2.14 (s, 3H), 2.09 (s, 3H). |
| | 2-(((1H-indol-2-yl)methyl)amino)-N-(3- | |
| | hydroxy-2, 6-dimethylphenyl)thiazole-5- | |
| | carboxamide | |
| BAA-358 | 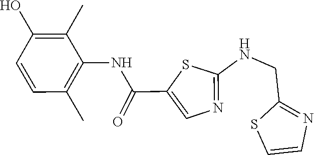 [SMILES: Cc1ccc(O)c(C)c1NC(=O)c1cnc(NCc2nccs2)s1] | MS (ES+) m/z 361 (M + H). ¹H NMR (300 MHz, Methanol-d4) δ 7.88 (s, 1H), 7.75 (d, J = 3.3 Hz, 1H), 7.54 (d, J = 3.3 Hz, 1H), 6.90 (dt, J = 8.2, 0.7 Hz, 1H), 6.67 (d, J = 8.2 Hz, 1H), 4.91 (s, 2H), 2.16-2.11 (m, 3H), 2.09 (s, 3H). |
| | N-(3-hydroxy-2,6-dimethylphenyl)-2- | |
| | ((thiazol-2-ylmethyl)amino)thiazole-5- | |
| | carboxamide | |
| BAA-360 | 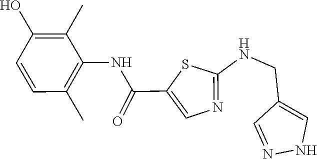 [SMILES: Cc1ccc(O)c(C)c1NC(=O)c1cnc(NCc2cn[nH]c2)s1] | MS (ES+) m/z 344 (M + H). ¹H NMR (300 MHz, Methanol-d4) δ 7.89 (s, 1H), 7.64 (s, 2H), 6.92-6.84 (m, 1H), 6.67 (d, J = 8.2 Hz, 1H), 4.45 (s, 2H), 2.15-2.12 (m, 3H), 2.09 (s, 3H). |
| | 2-(((1H-pyrazol-4-yl)methyl)amino)-N-(3- | |
| | hydroxy-2,6-dimethylphenyl) thiazole-5- | |
| | carboxamide | |
| BAA-362 | 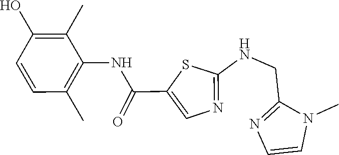 [SMILES: Cc1ccc(O)c(C)c1NC(=O)c1cnc(NCc2nccn2C)s1] | MS (ES+) m/z 358 (M + H). ¹H NMR (300 MHz, Methanol-d4) δ 7.92 (s, 1H), 7.09 (s, 1H), 6.92 (d, J = 6.0 Hz, 2H), 6.69 (d, J = 8.2 Hz, 1H), 4.68 (s, 2H), 3.76 (s, 3H), 2.16 (s, 3H), 2.11 (s, 3H). |
| | N-(3-hydroxy-2,6-dimethylphenyl)-2-(((1- | |
| | methyl-1H-imidazol-2-yl)methyl)amino) | |
| | thiazole-5-carboxamide | |
| BAA-363 | 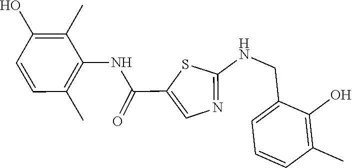 [SMILES: Cc1cccc(CNc2ncc(C(=O)Nc3c(C)ccc(O)c3C)s2)c1O] | MS (ES+) m/z 384 (M + H). ¹H NMR (300 MHz, Methanol-d4) δ 7.90 (s, 1H), 7.08 (t, J = 6.8 Hz, 2H), 6.92 (d, J = 8.2 Hz, 1H), 6.78 (t, J = 7.5 Hz, 1H), 6.69 (d, J = 8.2 Hz, 1H), 4.51 (s, 2H), 2.25 (s, 3H), 2.16 (s, 3H), 2.11 (s, 3H). |
| | N-(3-hydroxy-2,6-dimethylphenyl)-2-((2- | |
| | hydroxy-3-methylbenzyl)amino)thiazole- | |
| | 5-carboxamide | |
| BAA-364 | 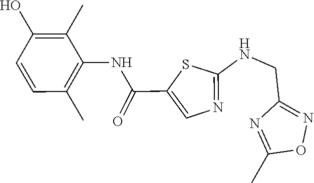 [SMILES: Cc1nc(CNc2ncc(C(=O)Nc3c(C)ccc(O)c3C)s2)no1] | MS (ES+) m/z 360 (M + H). ¹H NMR (300 MHz, Methanol-d4) δ 7.86 (s, 1H), 6.90 (d, J = 8.2 Hz, 1H), 6.67 (d, J = 8.2 Hz, 1H), 4.67 (s, 2H), 2.59 (s, 3H), 2.17-2.11 (m, 3H), 2.09 (s, 3H). |
| | N-(3-hydroxy-2,6-dimethylphenyl)-2-(((5- | |
| | methyl-1,2,4-oxadiazol-3-yl)methyl) | |
| | amino) thiazole-5-carboxamide | |

### N-(3-Hydroxy-2,6-dimethyl-phenyl)-2-(4-piperidylmethylamino)thiazole-5-carboxamide (BAA-079)

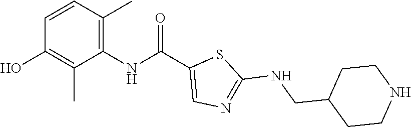 [SMILES: Cc1ccc(O)c(C)c1NC(=O)c1cnc(NCC2CCNCC2)s1]

Prepared as described in Method E from tert-butyl 4-[5-[(3-hydroxy-2,6-dimethyl-phenyl) carbamoyl]thiazol-2-yl]amino]methyl]piperidine-1-carboxylate (BAA-066)(15 mg, 0.0326 mmol, 1.0 eq) in HCl (4M in 1,4-dioxane, 3 mL, 12 mmol, 367 eq) for 4 h to afford the title compound (HCl salt; 13 mg, 0.0328 mmol, 100%). MS (ES+) m/z 361 (M+H). ¹H NMR (300 MHz, Methanol-d4) δ 8.11 (s, 1H), 6.93 (d, J=8.3 Hz, 1H), 6.71 (d, J=8.2 Hz, 1H), 3.47 (dd, J=14.0, 6.1 Hz, 3H), 3.05 (t, J=12.7 Hz, 2H), 2.12 (d, J=16.4 Hz, 8H), 1.66-1.24 (m, 4H).

5. Synthesis of Pyrazole-Pyrrolidine Analogues. Derivatisation of BAA-056 Amide Coupling of BAA-056 with Acid Chlorides

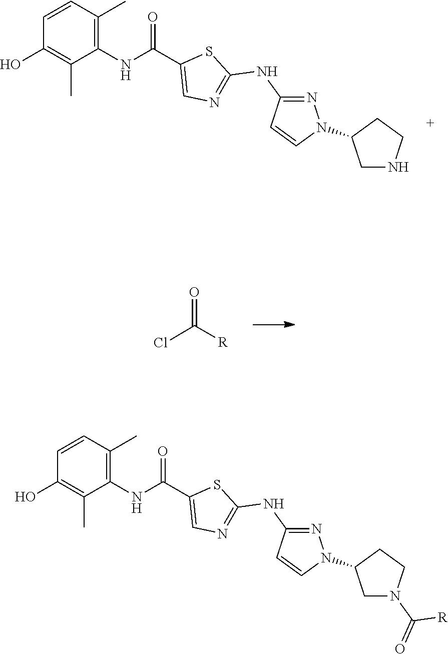

General Method G: Amide Coupling of BAA-056 with Acid Chlorides

DIPEA (2.5-4.0 eq) was added to a solution of N-(3-hydroxy-2,6-dimethyl-phenyl)-2-[[1-[(3R)-pyrrolidin-3-yl]pyrazol-3-yl]amino]thiazole-5-carboxamide hydrochloride (BAA-056)(1.0 eq) and acid chloride (1.0-1.3 eq) in anhydrous DCM (0.03-0.08 M) at 0° C. or RT under nitrogen. The mixture was stirred at RT or 40° C. for 18 h. The reaction was monitored by consumption of starting material via UPLC. The crude material was purified by normal phase chromatography (SiO₂) using a gradient of MeOH: DCM or MeOH/EtOAc.

### 2-[[1-[(3R)-1-(2.2-Dimethylpropanoyl)pyrrolidin-3-yl]pyrazol-3-yl]amino]-N-(3-hydroxy-2,6-dimethyl-phenyl)thiazole-5-carboxamide (BAA-080)

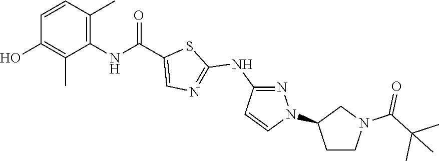 [SMILES: Cc1ccc(O)c(C)c1NC(=O)c1cnc(Nc2ccn([C@@H]3CCN(C(=O)C(C)(C)C)C3)n2)s1]

Prepared as described in Method G from N-(3-hydroxy-2,6-dimethyl-phenyl)-2-[1-[(3R)-pyrrolidin-3-yl]pyrazol-3-yl]amino]thiazole-5-carboxamide hydrochloride (BAA-056)(15 mg, 0.035 mmol, 1.0 eq), trimethylacetyl chloride (5 μL, 0.041 mmol, 1.2 eq) and DIPEA (15 μL, 0.086 mmol) in anhydrous DCM (0.6 mL) to afford the title compound (5 mg, 0.010 mmol, 30%) as a white solid after normal phase chromatography (SiO₂) eluting with 0-20% MeOH: DCM. MS (ES+) m/z 483.3 (M+H). ¹H NMR (Methanol-d₄, 300 MHz) δ 7.94 (s, 1H), 7.52 (d, 1H, J=2.0 Hz), 6.91 (d, 1H, J=8.2 Hz), 6.68 (d, 1H, J=8.2 Hz), 6.31 (s, 1H), 5.13-5.05 (m, 1H), 4.12-3.77 (m, 4H), 2.43-2.29 (m, 2H), 2.14 (s, 3H), 2.09 (s, 3H), 1.26 (s, 9H) ppm.

The following example compounds were prepared similarly using Method G and the appropriate acid chloride:

| # | Structure & Name | Analytical Data |
|---|---|---|
| BAA- 081 | 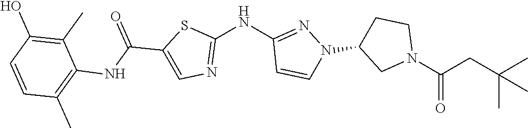 [SMILES: Cc1ccc(O)c(C)c1NC(=O)c1cnc(Nc2ccn([C@@H]3CCN(C(=O)CC(C)(C)C)C3)n2)s1] | MS (ES+) m/z 497.3 (M + H). ¹H NMR (Methanol-d₄, 300 MHz) δ 7.94 (d, 1H, J = 1.3 Hz), 7.51 (dd, 1H, J = 3.4, 2.1 Hz), 6.91 (d, 1H, J = 8.2 Hz), 6.68 (d, 1H, J = 8.2 Hz), 6.30 (s, 1H), 5.17-5.10 (m, 1H), 4.02- 3.83 (m, 2H), 3.77-3.51 (m, 2H), 2.45-2.34 (m, 2H), 2.29-2.24 (m, 2H), 2.14 (s, 3H), 2.09 (s, 3H), 1.06 (d, 9H, J = 12.1 Hz) ppm. |
| | 2-[[1-[(3R)-1-(3,3- | |
| | dimethylbutanoyl) pyrrolidin-3-yl]pyrazol- | |
| | 3-yl]amino]-N-(3-hydroxy-2,6-dimethyl- | |
| | phenyl)thiazole-5-carboxamide | |
| BAA- 082 | 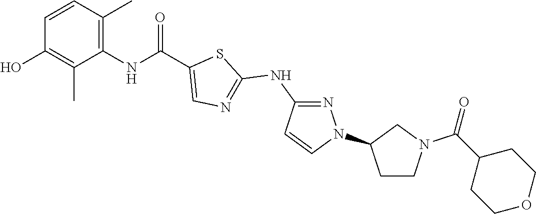 [SMILES: Cc1ccc(O)c(C)c1NC(=O)c1cnc(Nc2ccn([C@@H]3CCN(C(=O)C4CCOCC4)C3)n2)s1] | MS (ES+) m/z 511.3 (M + H). ¹H NMR (Methanol-d₄, 300 MHz) δ 7.94 (d, 1H, J = 2.3 Hz), 7.52 (t, 1H, J = 2.4 Hz), 6.91 (d, 1H, J = 8.3 Hz), 6.68 (d, 1H, J = 8.2 Hz), 6.30 (s, 1H), 5.16 (dt, 1H, J = 20.3, 6,1 Hz), 4.04-3.71 (m, 6H), 3.55-3.43 (m, 2H), 2.89- 2.70 (m, 1H), 2.40 (dq, 2H, J = 27.5, 7.1 Hz), 2.14 (s, 3H), 2.09 (s, 3H), 1.81-1.63 (m, 4H) ppm. |
| | N-(3-hydroxy-2,6-dimethyl-phenyl)-2- | |
| | [1-[(3R)-1-(tetrahydropyran-4- | |
| | carbonyl)pyrrolidin-3-yl]pyrazol-3- | |
| | yl]amino]thiazole-5-carboxamide | |
| BAA- 083 | 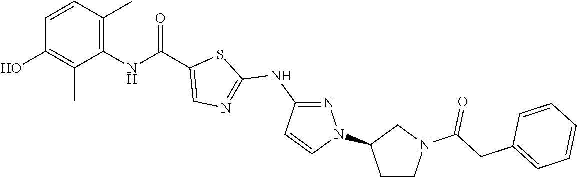 [SMILES: Cc1ccc(O)c(C)c1NC(=O)c1cnc(Nc2ccn([C@@H]3CCN(C(=O)Cc4ccccc4)C3)n2)s1] | MS (ES+) m/z 517.3 (M + H). ¹H NMR (Methanol-d₄, 300 MHz) δ 7.93 (s, 1H), 7.52 (d, 1H, J = 2.0 Hz), 7.32-7.22 (m, 5H), 6.91 (d, 1H, J = 8.2 Hz), 6.68 (d, 1H, J = 8.2 Hz), 6.29 (dd, 1H, J = 3.1, 2,1 Hz), 5.14 (dp, 1H, J = 12.7, 6,1 Hz), 3.99-3.80 (m, 3H), 3.77-3.68 (m, 2H), 3.62-3.53 (m, 1H), 2.44- 2.31 (m, 2H), 2.14 (d, 3H, J = 1.5 Hz), 2.09 (d, 3H, J = 1.7 Hz) ppm. |
| | N-(3-hydroxy-2,6-dimethyl-phenyl)-2- | |
| | [ 1-[(3R)-1-(2-phenylacetyl)pyrrolidin-3- | |
| | yl]pyrazol-3-yl]amino]thiazole-5- | |
| | carboxamide | |
| BAA- 084 | 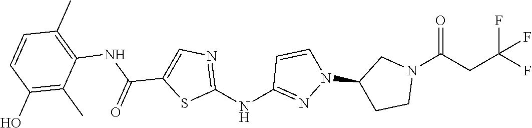 [SMILES: Cc1ccc(O)c(C)c1NC(=O)c1cnc(Nc2ccn([C@@H]3CCN(C(=O)CC(F)(F)F)C3)n2)s1] | MS (ES+) m/z 509.3 (M + H). ¹H NMR (Methanol-d₄, 300 MHz) δ 7.94 (d, 1H, J = 1.1 Hz), 7.53 (t, 1H, J = 2.0 Hz), 6.91 (d, 1H, J = 8.2 Hz), 6.68 (d, 1H, J = 8.2 Hz), 6.30 (s, 1H), 5.24-5.15 (m, 1H), 4.02-3.58 (m, 4H), 3.52-3.37 (m, 2H), 2.48-2.31 (m, 2H), 2.14 (s, 3H), 2.09 (s, 3H) ppm. ¹⁹F NMR (Methanol-d₄, 282 MHz) δ −64.13 ppm. |
| | N-(3-hydroxy-2,6-dimethyl-phenyl)-2- | |
| | [1-[(3R)-1-(3,3,3- | |
| | trifluoropropanoyl) pyrrolidin-3- | |
| | yl]pyrazol-3-yl]amino]thiazole-5- | |
| | carboxamide | |
| BAA- 085 | 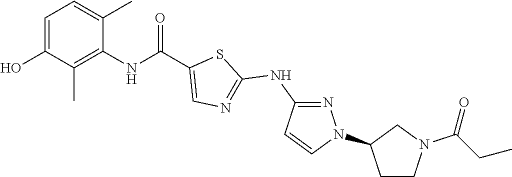 [SMILES: CCC(=O)N1CC[C@@H](n2ccc(Nc3ncc(C(=O)Nc4c(C)ccc(O)c4C)s3)n2)C1] | MS (ES+) m/z 455.3 (M + H). ¹H NMR (Methanol-d₄, 300 MHz) δ 7.94 (d, 1H, J = 1.3 Hz), 7.52 (t, 1H, J = 2.3 Hz), 6.91 (d, 1H, J = 8.3 Hz), 6.68 (d, 1H, J = 8.2 Hz), 6.31 (s, 1H), 5.16 (dt, 1H, J = 19.5, 6.1 Hz), 3.98-3.51 (m, 4H), 2.47-2.31 (m, 4H), 2.14 (s, 3H), 2.09 (s, 3H), 1.13 (td, 3H, J = 7.5, 6.0 Hz) ppm. |
| | N-(3-hydroxy-2,6-dimethyl-phenyl)-2- | |
| | [ 1-[(3R)-1-propanoylpyrrolidin-3- | |
| | yl]pyrazol-3-yl]amino]thiazole-5- | |
| | carboxamide | |
| BAA- 086 | 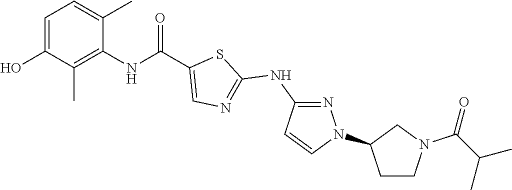 [SMILES: Cc1ccc(O)c(C)c1NC(=O)c1cnc(Nc2ccn([C@@H]3CCN(C(=O)C(C)C)C3)n2)s1] | MS (ES+) m/z 469.3 (M + H). ¹H NMR (Methanol-d₄, 300 MHz) δ 7.94 (d, 1H, J = 1.6 Hz), 7.53-7.51 (m, 1H), 6.91 (d, 1H, J = 8.2 Hz), 6.68 (d, 1H, J = 8.2 Hz), 6.31 (s, 1H), 5.16 (dt, 1H, J = 19.6, 6.0 Hz), 4.04- 3.50 (m, 4H), 2.78 (dp, 1H, J = 28.2, 6.8 Hz), 2.40 (dq, 2H, J = 28.5, 7.2 Hz), 2.14 (s, 3H), 2.09 (s, 3H), 1.15-1.09 (m, 6H) ppm. |
| | N-(3-hydroxy-2,6-dimethyl-phenyl)-2- | |
| | [1-[(3R)-1-(2- | |
| | methylpropanoyl) pyrrolidin-3-yl]pyrazol- | |
| | 3-yl]amino]thiazole-5-carboxamide | |
| BAA- 087 | 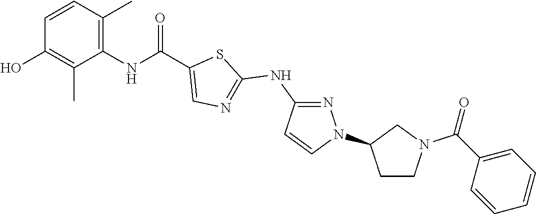 [SMILES: Cc1ccc(O)c(C)c1NC(=O)c1cnc(Nc2ccn([C@@H]3CCN(C(=O)c4ccccc4)C3)n2)s1] | MS (ES+) m/z 503.3 (M + H). ¹H NMR (Methanol-d₄, 300 MHz) δ 7.91 (d, 1H, J = 19.8 Hz), 7.59-7.42 (m, 6H), 6.91 (d, 1H, J = 8.2 Hz), 6.68 (d, 1H, J = 8.2 Hz), 6.30 (dd, 1H, J = 17.7, 1.6 Hz), 5.16 (dp, 1H, J = 47.7, 5.5 Hz), 4.11-3.57 (m, 4H), 2.47 (dt, 1H, J = 14.1, 6.4 Hz), 2.35 (q, 1H, J = 7.2 Hz), 2.14 (d, 3H, J = 3.8 Hz), 2.09 (d, 3H, J = 3.9 Hz) ppm. |
| | 2-[1-[(3R)-1-benzoylpyrrolidin-3- | |
| | yl]pyrazol-3-yl]amino]-N-(3-hydroxy-2,6- | |
| | dimethyl-phenyl)thiazole-5-carboxamide | |
| BAA- 088 | 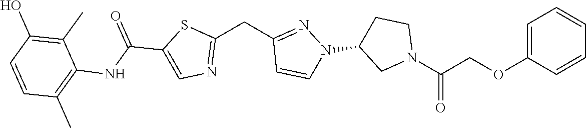 [SMILES: Cc1ccc(O)c(C)c1NC(=O)c1cnc(Cc2ccn([C@@H]3CCN(C(=O)COc4ccccc4)C3)n2)s1] | MS (ES+) m/z 533.3 (M + H). ¹H NMR (Methanol-d₄, 300 MHz) δ 7.94 (s, 1H), 7.54 (t, 1H, J = 1.8 Hz), 7.31-7.24 (m, 2H), 7.00-6.90 (m, 4H), 6.68 (d, 1H, J = 8.2 Hz), 6.31 (s, 1H), 5.19 (dt, 1H, J = 18.9, 5.6 Hz), 4.75 (d, 2H, J = 16.7 Hz), 4.05-3.71 (m, 4H), 2.40 (dq, 2H, J = 34.6, 7.1 Hz), 2.14 (s, 3H), 2.09 (s, 3H) ppm. |
| | N-(3-hydroxy-2,6-dimethyl-phenyl)-2- | |
| | [1-[(3R)-1-(2-phenoxyacetyl)pyrrolidin- | |
| | 3-yl]pyrazol-3-yl]amino]thiazole-5- | |
| | carboxamide | |
| BAA- 089 | 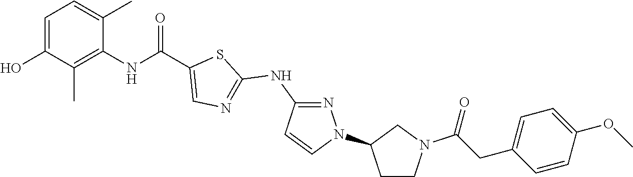 [SMILES: COc1ccc(CC(=O)N2CC[C@@H](n3ccc(Nc4ncc(C(=O)Nc5c(C)ccc(O)c5C)s4)n3)C2)cc1] | MS (ES+) m/z 547.3 (M + H). ¹H NMR (Methanol-d₄, 300 MHz) δ 7.93 (d, 1H, J = 1.4 Hz), 7.52 (d, 1H, J = 2.0 Hz), 7.22-7.13 (m, 2H), 6.92-6.83 (m, 3H), 6.68 (d, 1H, J = 8.2 Hz), 6.30 (dd, 1H, J = 3.2, 2.1 Hz), 5.19-5.07 (m, 1H), 3.94-3.70 (m, 7H), 3.65 (d, 2H, J = 15.9 Hz), 2.44-2.30 (m, 2H), 2.14 (d, 3H, J = 1.4 Hz), 2.09 (d, 3H, J = 1.6 Hz) ppm. |
| | N-(3-hydroxy-2,6-dimethyl-phenyl)-2- | |
| | [ 1-[(3R)-1-[2-(4- | |
| | methoxyphenyl)acetyl]pyrrolidin-3- | |
| | yl]pyrazol-3-yl]amino]thiazole-5- | |
| | carboxamide | |
| BAA- 090 | 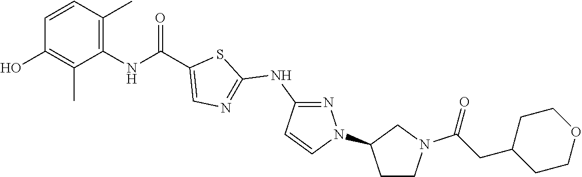 [SMILES: Cc1ccc(O)c(C)c1NC(=O)c1cnc(Nc2ccn([C@@H]3CCN(C(=O)CC4CCOCC4)C3)n2)s1] | MS (ES+) m/z 525.3 (M + H). ¹H NMR (Methanol-d₄, 300 MHz) δ 7.94 (d, 1H, J = 2.1 Hz), 7.53-7.51 (m, 1H), 6.91 (d, 1H, J = 8.2 Hz), 6.68 (d, 1H, J = 8.2 Hz), 6.31 (s, 1H), 5.22-5.08 (m, 1H), 3.96-3.65 (m, 6H), 3.48-3.37 (m, 2H), 2.46-2.18 (m, 5H), 2.14 (s, 3H), 2.09 (s, 3H), 1.75-1.64 (m, 2H), 1.39- 1.29 (m, 2H) ppm. |
| | N-(3-hydroxy-2,6-dimethyl-phenyl)-2- | |
| | [1-[(3R)-1-(2-tetrahydropyran-4- | |
| | ylacetyl)pyrrolidin-3-yl]pyrazol-3- | |
| | yl]amino]thiazole-5-carboxamide | |
| BAA- 091 | 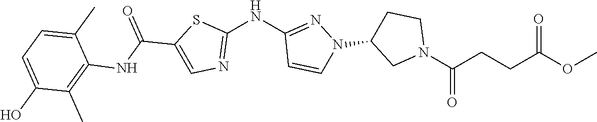 [SMILES: COC(=O)CCC(=O)N1CC[C@@H](n2ccc(Nc3ncc(C(=O)Nc4c(C)ccc(O)c4C)s3)n2)C1] | MS (ES+) m/z 513.3 (M + H). ¹H NMR (Methanol-d₄, 300 MHz) δ 7.94 (d, 1H, J = 2.5 Hz), 7.53 (dd, 1H, J = 3.2, 2.1 Hz), 6.91 (d, 1H, J = 8.7 Hz), 6.68 (d, 1H, J = 8.2 Hz), 6.31 (t, 1H, J = 2.1 Hz), 5.17 (dp, 1H, J = 23.3, 6.3 Hz), 4.04-3.69 (m, 4H), 3.67 (d, 3H, J = 2.2 Hz), 2.69-2.62 (m, 4H), 2.40 (dq, 2H, J = 31.0, 7.2 Hz), 2.14 (s, 3H), 2.09 (s, 3H) ppm. |
| | methyl 4-[(3R)-3-[3-[5-[(3-hydroxy-2,6- | |
| | dimethyl-phenyl)carbamoyl]thiazol-2- | |
| | yl]amino]pyrazol-1-yl]pyrrolidin-1-yl]-4- | |
| | oxo-butanoate | |
| BAA- 092 | 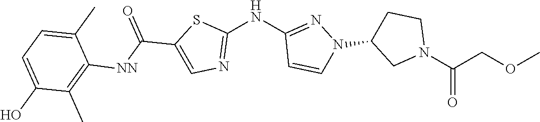 | MS (ES+) m/z 471.3 (M + H). ¹H NMR (Methanol-d₄, 300 MHz) δ 7.94 (d, 1H, J = 1.1 Hz), 7.52 (t, 1H, J = 1.8 Hz), 6.91 (d, 1H, J = 8.2 Hz), 6.68 (d, 1H, J = 8.2 Hz), 6.31 (s, 1H), 5.22-5.12 (m, 1H), 4.12 (d, 2H, J = 18.5 Hz), 3.94-3.76 (m, 3H), 3.67-3.58 (m, 1H), 3.42 (d, 3H, J = 9.9 Hz), 2.38 (dq, 2H, J = 27.9, 7.2 Hz), 2.14 (s, 3H), 2.09 (s, 3H) ppm. |
| | N-(3-hydroxy-2,6-dimethyl-phenyl)-2- | |
| | [1-[(3R)-1-(2-methoxyacetyl)pyrrolidin- | |
| | 3-yl]pyrazol-3-yl]amino]thiazole-5- | |
| | carboxamide | |
| BAA- 093 | 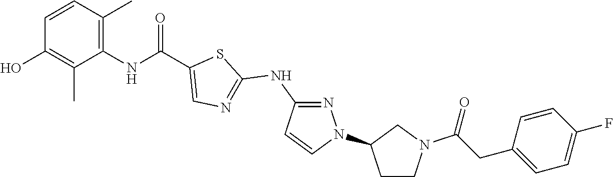 [SMILES: Cc1ccc(O)c(C)c1NC(=O)c1cnc(Nc2ccn([C@@H]3CCN(C(=O)Cc4ccc(F)cc4)C3)n2)s1] | MS (ES+) m/z 535.9 (M + H). ¹H NMR (Methanol-d₄, 300 MHz) δ 7.93 (d, 1H, J = 1.6 Hz), 7.52 (dd, 1H, J = 1.9, 1.2 Hz), 7.33-7.23 (m, 2H), 7.08-6.99 (m, 2H), 6.91 (d, 1H, J = 8.3 Hz), 6.68 (d, 1H, J = 8.2 Hz), 6.30 (t, 1H, J = 2.1 Hz), 5.22-5.09 (m, 1H), 4.01- 3.69 (m, 6H), 2.46-2.32 (m, 2H), 2.14 (s, 3H), 2.09 (s, 3H) ppm. ¹⁹F NMR (Methanol-d₄, 282 MHz) δ −118.5 ppm. |
| | 2-[1-[(3R)-1-[2-(4- | |
| | fluorophenyl)acetyl]pyrrolidin-3- | |
| | yl]pyrazol-3-yl]amino]-N-(3-hydroxy-2,6- | |
| | dimethyl-phenyl)thiazole-5-carboxamide | |
| BAA- 094 | 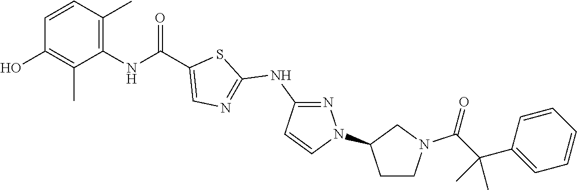 [SMILES: Cc1ccc(O)c(C)c1NC(=O)c1cnc(Nc2ccn([C@@H]3CCN(C(=O)C(C)(C)c4ccccc4)C3)n2)s1] | MS (ES+) m/z 545.4 (M + H). ¹H NMR (Methanol-d₄, 300 MHz) δ 7.92 (s, 1H), 7.46 (dd, 1H, J = 16.6, 1.9 Hz), 7.37 (t, 1H, J = 7.7 Hz), 7.26 (dd, 2H, J = 16.2, 7.6 Hz), 7.17-7.13 (m, 2H), 6.91 (dd, 1H, J = 8.2, 4.5 Hz), 6.68 (dd, 1H, J = 8.2, 3.4 Hz), 6.24 (dd, 1H, J = 22.8, 1.8 Hz), 4.85 (dt, 1H, J = 75.0, 5.8 Hz), 3.96-3.56 (m, 2H), 3.26-3.18 (m, 0.5H), 3.11 (d, 1H, J = 5.8 Hz), 2.98-2.90 (m, 0.5H), 2.40-2.20 (m, 1H), 2.15 (d, 3H, J = 9.3 Hz), 2.10 (d, 3H, J = 9.2 Hz), 2.04-1.96 (m, 1H), 1.55 (d, 3H, J = 10.0 Hz), 1.47 (d, |
| | | 3H, J = 7.9 Hz) ppm. |
| | N-(3-hydroxy-2,6-dimethyl-phenyl)-2- | |
| | [1-[(3R)-1-(2-methyl-2-phenyl- | |
| | propanoyl) pyrrolidin-3-yl]pyrazol-3- | |
| | yl]amino]thiazole-5-carboxamide | |
| BAA- 095 | 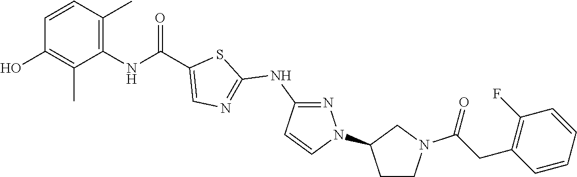 [SMILES: Cc1ccc(O)c(C)c1NC(=O)c1cnc(Nc2ccn([C@@H]3CCN(C(=O)Cc4ccccc4F)C3)n2)s1] | MS (ES+) m/z 535.3 (M + H). ¹H NMR (Methanol-d₄, 300 MHz) δ 7.94 (d, 1H, J = 1.7 Hz), 7.53 (t, 1H, J = 2.2 Hz), 7.34- 7.24 (m, 2H), 7.17-7.03 (m, 2H), 6.91 (d, 1H, J = 8.2 Hz), 6.68 (d, 1H, J = 8.2 Hz), 6.31 (d, 1H, J = 2.0 Hz), 5.25-5.11 (m, 1H), 4.07-3.78 (m, 4H), 3.75-3.55 (m, 2H), 2.41 (dq, 2H, J = 27.2, 7.3 Hz), 2.14 (s, 3H), 2.09 (s, 3H) ppm ¹⁹F NMR (Methanol-d₄, 282 MHz) δ −119.26 ppm. |
| | 2-[1-[(3R)-1-[2-(2- | |
| | fluorophenyl)acetyl]pyrrolidin-3- | |
| | yl]pyrazol-3-yl]amino]-N-(3-hydroxy-2,6- | |
| | dimethyl-phenyl)thiazole-5-carboxamide | |
| BAA- 096 | 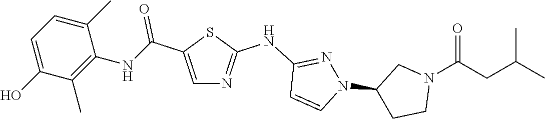 [SMILES: Cc1ccc(O)c(C)c1NC(=O)c1cnc(Nc2ccn([C@@H]3CCN(C(=O)CC(C)C)C3)n2)s1] | MS (ES+) m/z 483.3 (M + H). ¹H NMR (Methanol-d₄, 300 MHz) δ 7.94 (d, 1H, J = 1.6 Hz), 7.52 (dd, 1H, J = 3.2, 2.1 Hz), 6.91 (d, 1H, J = 8.3 Hz), 6.68 (d, 1H, J = 8.2 Hz), 6.30 (s, 1H), 5.19-5.10 (m, 1H), 4.00- 3.51 (m, 4H), 2.46-2.16 (m, 5H), 2.14 (s, 3H), 2.09 (s, 3H), 1.01-0.95 (m, 6H) ppm. |
| | N-(3-hydroxy-2,6-dimethyl-phenyl)-2- | |
| | [[1-[(3R)-1-(3-methylbutanoyl)pyrrolidin- | |
| | 3-yl]pyrazol-3-yl]amino]thiazole-5- | |
| | carboxamide | |
| BAA- 097 | 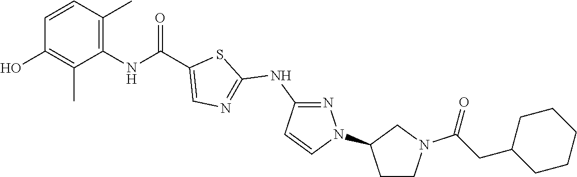 [SMILES: Cc1ccc(O)c(C)c1NC(=O)c1cnc(Nc2ccn([C@@H]3CCN(C(=O)CC4CCCCC4)C3)n2)s1] | MS (ES+) m/z 523.4 (M + H). ¹H NMR (Methanol-d₄, 300 MHz) δ 7.94 (d, 1H, J = 1.9 Hz), 7.52 (dd, 1H, J = 2.9, 2.2 Hz), 6.91 (d, 1H, J = 8.3 Hz), 6.68 (d, 1H, J = 8.2 Hz), 6.31 (s, 1H), 5.21-5.08 (m, 1H), 3.99- 3.50 (m, 4H), 2.46-2.32 (m, 2H), 2.27-2.18 (m, 2H), 2.14 (s, 3H), 2.09 (s, 3H), 1.81-1.68 (m, 5H), 1.38-0.94 (m, 6H) ppm. |
| | 2-[1-[(3R)-1-(2- | |
| | cyclohexylacetyl) pyrrolidin-3-yl]pyrazol- | |
| | 3-yl]amino]-N-(3-hydroxy-2,6-dimethyl- | |
| | phenyl)thiazole-5-carboxamide | |
| BAA- 098 | 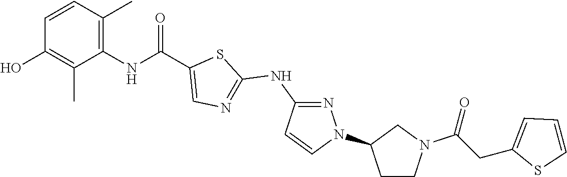 [SMILES: Cc1ccc(O)c(C)c1NC(=O)c1cnc(Nc2ccn([C@@H]3CCN(C(=O)Cc4cccs4)C3)n2)s1] | MS (ES+) m/z 523.2 (M + H). ¹H NMR (Methanol-d₄, 300 MHz) δ 7.93 (d, 1H, J = 1.4 Hz), 7.52 (t, 1H, J = 1.9 Hz), 7.30- 7.25 (m, 1H), 6.97-6.90 (m, 3H), 6.68 (d, 1H, J = 8.2 Hz), 6.30 (dd, 1H, J = 1.9, 1.2 Hz), 5.22-5.10 (m, 1H), 4.05-3.85 (m, 4H), 3.82- 3.54 (m, 2H), 2.47-2.32 (m, 2H), 2.14 (s, 3H), 2.09 (s, 3H) ppm. |
| | N-(3-hydroxy-2,6-dimethyl-phenyl)-2- | |
| | [ 1-[(3R)-1-[2-(2- | |
| | thienyl)acetyl]pyrrolidin-3-yl]pyrazol-3- | |
| | yl]amino]thiazole-5-carboxamide | |

Amide Coupling of BAA-056 with Carboxylic Acids

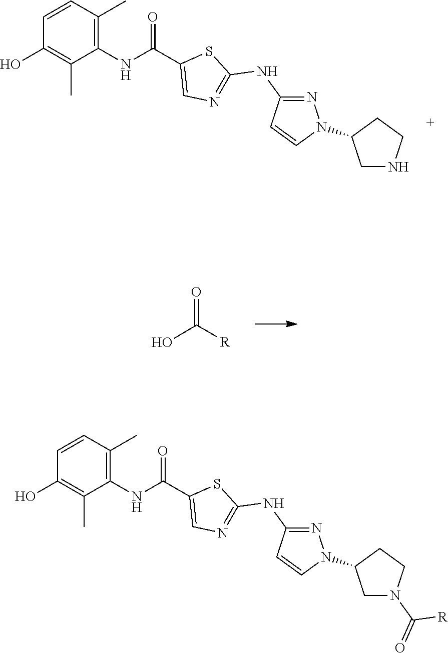

General Method H: Amide Coupling of BAA-056 with Carboxylic Acids

Thionyl chloride (1.2 eq) was added to a suspension of N-(3-hydroxy-2,6-dimethyl-phenyl)-2-[1-[(3R)-pyrrolidin-3-yl]pyrazol-3-yl]amino]thiazole-5-carboxamide hydrochloride (BAA-056)(1.0 eq), carboxylic acid (1.0-1.2 eq) and NEt₃ (4.0-6.0 eq) in anhydrous DCM (0.05-0.07 M) containing anhydrous DMF (1 drop) at RT under nitrogen. The mixture was stirred at RT for 18 h. Reaction monitoring by consumption of starting material via UPLC. The crude material was purified by normal phase chromatography (SiO₂) using a gradient of MeOH: DCM or MeOH/EtOAc.

### 2-[[1-[(3R)-1-(2,2-Difluoro-2-phenyl-acetyl)pyrrolidin-3-yl]pyrazol-3-yl]amino]-N-(3-hydroxy-2,6-dimethyl-phenyl)thiazole-5-carboxamide (BAA-099)

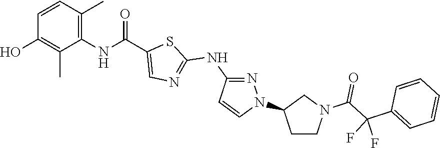 [SMILES: Cc1ccc(O)c(C)c1NC(=O)c1cnc(Nc2ccn([C@@H]3CCN(C(=O)C(F)(F)c4ccccc4)C3)n2)s1]

Prepared as described in Method H from N-(3-hydroxy-2,6-dimethyl-phenyl)-2-[1-[(3R)-pyrrolidin-3-yl]pyrazol-3-yl]amino]thiazole-5-carboxamide hydrochloride (BAA-056)(35 mg, 0.081 mmol, 1.0 eq), α,α-difluoro-phenyl-acetic acid (14 mg, 0.081 mmol, 1.0 eq), NEt₃ (50 μL, 0.36 mmol, 4.5 eq) and anhydrous DMF (1 drop) in anhydrous DCM (1.2 mL) to afford the title compound (14 mg, 0.025 mmol, 31%) as an off-white solid after normal phase chromatography eluting with 0-30% MeOH: DCM. MS (ES+) m/z 553.3 (M+H). ¹H NMR (Methanol-d₄, 300 MHz) δ 7.92 (d, 1H, J=6.7 Hz), 7.64-7.61 (m, 1H), 7.57-7.42 (m, 5H), 6.91 (dd, 1H, J=8.2, 1.8 Hz), 6.68 (dd, 1H, J=8.2, 1.4 Hz), 6.25 (d, 1H, J=20.7 Hz), 5.13-5.04 (m, 1H), 4.04-3.64 (m, 4H), 2.40-2.28 (m, 2H), 2.14 (d, 3H, J=2.9 Hz), 2.09 (d, 3H, J=3.0 Hz) ppm. ¹⁹F NMR (Methanol-d₄, 282 MHz) δ-98.4, -98.5 ppm.

The following example compounds were prepared similarly using Method H and the appropriate carboxylic acid:

| # | Structure & Name | Analytical Data |
|---|---|---|
| — | 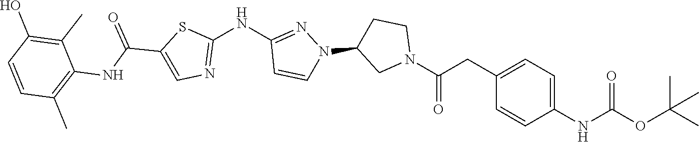 [SMILES: Cc1ccc(O)c(C)c1NC(=O)c1cnc(Nc2ccn([C@H]3CCN(C(=O)Cc4ccc(NC(=O)OC(C)(C)C)cc4)C3)n2)s1] | MS (ES+) m/z 632.3 (M + H). ¹H NMR (Methanol-d₄, 300 MHz) δ 7.93 (d, 1H, J = 1.0 Hz), 7.52 (d, 1H, J = 2.0 Hz), 7.35 (t, 2H, J = 8.7 Hz), 7.16 (dd, 2H, J = 14.8, 8.6 Hz), 6.91 (d, 1H, J = 8.6 Hz), 6.68 (d, 1H, J = 8.2 Hz), 6.30 (s, 1H), 5.18-5.10 (m, 1H), 3.99-3.53 (m, 6H), 2.44- 2.31 (m, 2H), 2.14 (s, 3H), 2.09 (s, 3H), 1.51 (d, 9H, J = 3.1 Hz) ppm. |
| | tert-butyl N-[4-[2-[(3R)-3-[3-[5-[(3- | |
| | hydroxy-2,6-dimethyl- | |
| | phenyl) carbamoyl]thiazol-2- | |
| | yl]amino]pyrazol-1-yl]pyrrolidin-1- | |
| | yl]-2-oxo-ethyl]phenyl]carbamate | |
| — | 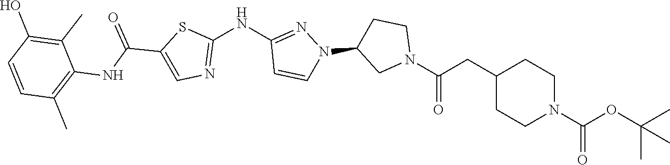 [SMILES: Cc1ccc(O)c(C)c1NC(=O)c1cnc(Nc2ccn([C@H]3CCN(C(=O)CC4CCN(C(=O)OC(C)(C)C)CC4)C3)n2)s1] | MS (ES+) m/z 624.4 (M + H). ¹H NMR (Methanol-d₄, 300 MHz) δ 7.94 (d, 1H, J = 1.4 Hz), 7.52 (t, 1H, J = 2.1 Hz), 6.91 (d, 1H, J = 8.2 Hz), 6.68 (d, 1H, J = 8.2 Hz), 6.31 (s, 1H), 5.20- 5.10 (m, 1H), 4.11-3.51 (m, 7H), 2.83-2.70 (m, 2H), 2.46-2.25 (m, 4H), 2.14 (s, 3H), 2.09 (s, 3H), 1.78- 1.69 (m, 2H), 1.45 (d, 9H, J = 4.6 Hz), 1.19-1.10 (m, 2H) ppm |
| | tert-butyl 4-[2-[(3R)-3-[3-[5-[(3- | |
| | hydroxy-2,6-dimethyl- | |
| | phenyl) carbamoyl]thiazol-2- | |
| | yllamino]pyrazol-1-yl]pyrrolidin-1- | |
| | yl]-2-oxo-ethyl]piperidine-1- | |
| | carboxylate | |
| BAA- 102 | 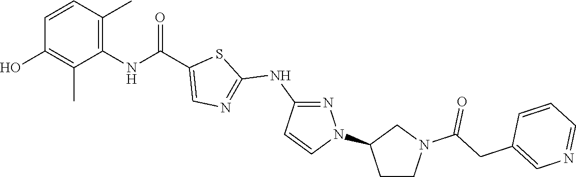 [SMILES: Cc1ccc(O)c(C)c1NC(=O)c1cnc(Nc2ccn([C@@H]3CCN(C(=O)Cc4cccnc4)C3)n2)s1] | MS (ES+) m/z 518.0 (M + H). ¹H NMR (Methanol-d₄, 300 MHz) δ 8.48-8.41 (m, 2H), 7.94 (d, 1H, J = 2.5 Hz), 7.82-7.74 (m, 1H), 7.53 (t, 1H, J = 1.9 Hz), 7.44-7.38 (m, 1H), 6.91 (d, 1H, J = 8.5 Hz), 6.68 (d, 1H, J = 8.2 Hz), 6.31 (s, 1H), 5.25-5.14 (m, 1H), 4.06-3.56 (m, 6H), 2.50- 2.33 (m, 2H), 2.14 (s, 3H), 2.09 (s, 3H) ppm. |
| BAA- 103 | 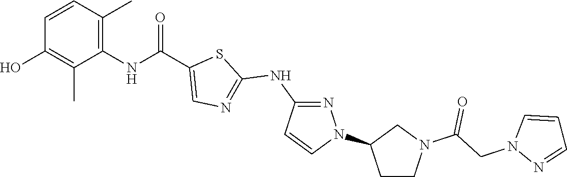 [SMILES: Cc1ccc(O)c(C)c1NC(=O)c1cnc(Nc2ccn([C@@H]3CCN(C(=O)Cn4cccn4)C3)n2)s1] | MS (ES+) m/z 507.0 (M + H). ¹H NMR (Methanol-d₄, 300 MHz) δ 7.94 (d, 1H, J = 4.3 Hz), 7.66 (dd, 1H, J = 5.1, 2.4 Hz), 7.55-7.51 (m, 2H), 6.91 (d, 1H, J = 8.6 Hz), 6.68 (d, 1H, J = 8.2 Hz), 6.35-6.31 (m, 2H), 5.27-5.16 (m, 1H), 5.09 (d, 2H, J = 15.5 Hz), 4.03-3.57 (m, 4H), 2.48 (q, 1H, J = 7.2 Hz), 2.36 (q, 1H, J = 7.2 Hz), 2.14 (s, 3H), 2.09 (s, 3H) ppm. |
| | N-(3-hydroxy-2,6-dimethyl- | |
| | phenyl)-2-[1-[(3R)-1-(2-pyrazol-1- | |
| | ylacetyl)pyrrolidin-3-yl]pyrazol-3- | |
| | yllamino]thiazole-5-carboxamide | |
| BAA- 104 | 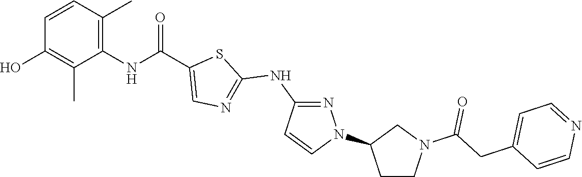 [SMILES: Cc1ccc(O)c(C)c1NC(=O)c1cnc(Nc2ccn([C@@H]3CCN(C(=O)Cc4ccncc4)C3)n2)s1] | MS (ES+) m/z 518.0 (M + H). ¹H NMR (Methanol-d₄, 300 MHz) δ 8.47 (dd, 2H, J = 8.1, 6.2 Hz), 7.94 (s, 1H), 7.53-7.52 (m, 1H), 7.38 (dd, 2H, J = 13.9, 6.1 Hz), 6.91 (d, 1H, J = 8.6 Hz), 6.68 (d, 1H, J = 8.2 Hz), 6.30 (s, 1H), 5.24-5.14 (m, 1H), 4.05-3.56 (m, 6H), 2.49-2.32 (m, 2H), 2.14 (s, 3H), 2.09 (s, 3H) ppm. |
| | N-(3-hydroxy-2,6-dimethyl- | |
| | phenyl)-2-[1-[(3R)-1-[2-(4- | |
| | pyridyl)acetyl]pyrrolidin-3- | |
| | yl]pyrazol-3-ylamino]thiazole-5- | |
| | carboxamide | |
| BAA- 105 | 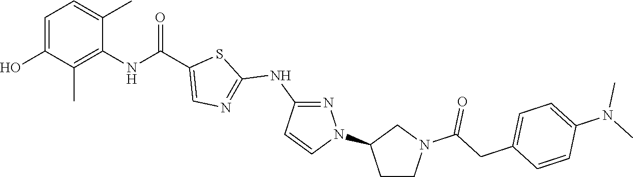 [SMILES: Cc1ccc(O)c(C)c1NC(=O)c1cnc(Nc2ccn([C@@H]3CCN(C(=O)Cc4ccc(N(C)C)cc4)C3)n2)s1] | MS (ES+) m/z 560.0 (M + H). ¹H NMR (Methanol-d₄, 300 MHz) δ 7.93 (s, 1H), 7.51 (d, 1H, J = 1.8 Hz), 7.13 (d, 1H, J = 8.8 Hz), 7.07 (d, 1H, J = 8.8 Hz), 6.91 (d, 1H, J = 8.2 Hz), 6.77-6.67 (m, 3H), 6.29 (s, 1H), 5.17-5.06 (m, 1H), 3.96-3.54 (m, 6H), 2.90 (s, 3H), 2.88 (s, 3H), 2.43- 2.30 (m, 2H), 2.14 (s, 3H), 2.09 (s, 3H) ppm. |
| | 2-[[ 1-[(3R)-1-[2-[4- | |
| | (dimethylamino)phenyl]acetyl]pyrr | |
| | olidin-3-yl]pyrazol-3-yl]amino]-N- | |
| | (3-hydroxy-2,6-dimethyl- | |
| | phenyl) thiazole-5-carboxamide | |

### Boc Deprotections:

### 2-[[1-[(3R)-1-[2-(4-Aminophenyl)acetyl]pyrrolidin-3-yl]pyrazol-3-yl]amino]-N-(3-hydroxy-2,6-dimethyl-phenyl)thiazole-5-carboxamide (BAA-106)

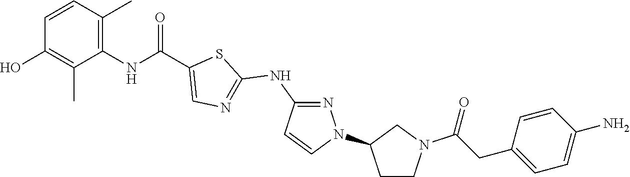 [SMILES: Cc1ccc(O)c(C)c1NC(=O)c1cnc(Nc2ccn([C@@H]3CCN(C(=O)Cc4ccc(N)cc4)C3)n2)s1]

Prepared as described in Method E from tert-butyl N-[4-[2-[(3R)-3-[3-[5-[(3-hydroxy-2,6-dimethyl-phenyl) carbamoyl]thiazol-2-yl]amino]pyrazol-1-yl]pyrrolidin-1-yl]-2-oxo-ethyl]phenyl]carbamate (9 mg, 0.014 mmol, 1.0 eq) in HCl (4 M in 1,4-dioxane, 0.1 mL, 0.4 mmol, 25 eq) at at 0° C. for 3 h to afford the title compound (BAA-106)(HCl salt; 7 mg, 0.012 mmol, 86%) as a pale pink solid. MS (ES+) m/z 532.3 (M+H). ¹H NMR (Methanol-d₄, 300 MHz) δ 7.96 (s, 1H), 7.58 (s, 1H), 7.49-7.31 (m, 4H), 6.91 (d, 1H, J=8.8 Hz), 6.69 (d, 1H, J=8.2 Hz), 6.34 (dd, 1H, J=3.9, 2.1 Hz), 5.26-5.17 (m, 1H), 4.08-3.71 (m, 6H), 2.50-2.35 (m, 2H), 2.14 (s, 3H), 2.09 (s, 3H) ppm.

### N-(3-Hydroxy-2,6-dimethyl-phenyl)-2-[[1-[(3R)-1-[2-(4-piperidyl)acetyl]pyrrolidin-3-yl]pyrazol-3-yl]amino]thiazole-5-carboxamide (BAA-107)

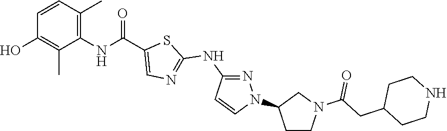 [SMILES: Cc1ccc(O)c(C)c1NC(=O)c1cnc(Nc2ccn([C@@H]3CCN(C(=O)CC4CCNCC4)C3)n2)s1]

Prepared as described in Method E from tert-butyl N-[4-[2-[(3R)-3-[3-[5-[(3-hydroxy-2,6-dimethyl-phenyl) carbamoyl]thiazol-2-yl]amino]pyrazol-1-yl]pyrrolidin-1-yl]-2-oxo-ethyl]phenyl]carbamate to afford the title compound (BAA-107)(HCl salt; 7 mg, 0.013 mmol, 97%) as an off-white solid. MS (ES+) m/z 524.3 (M+H). ¹H NMR (Methanol-d₄, 300 MHz) δ 8.05 (d, 1H, J=1.2 Hz), 7.81 (d, 1H, J=1.1 Hz), 6.92 (d, 1H, J=8.3 Hz), 6.70 (d, 1H, J=8.2 Hz), 6.47 (d, 1H, J=2.5 Hz), 5.40-5.25 (m, 1H), 4.07-3.53 (m, 5H), 3.41-3.35 (m, 2H), 3.07-2.96 (m, 2H), 2.51-2.36 (m, 4H), 2.15 (s, 3H), 2.09 (s, 3H), 2.05-1.98 (m, 2H), 1.57-1.44 (m, 2H) ppm.

Urea Formation from BAA-056 with Isocyanates

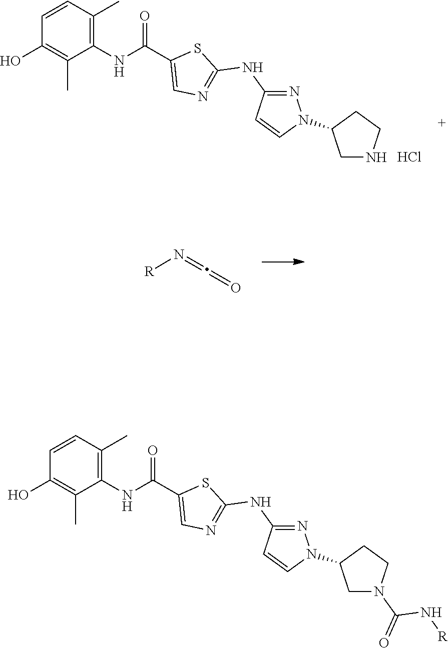

### 2-[[1-[(3R)-1-(Benzylcarbamoyl)pyrrolidin-3-yl]pyrazol-3-yl]amino]-N-(3-hydroxy-2,6-dimethyl-phenyl)thiazole-5-carboxamide (BAA-108)

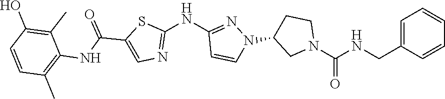 [SMILES: Cc1ccc(O)c(C)c1NC(=O)c1cnc(Nc2ccn([C@@H]3CCN(C(=O)NCc4ccccc4)C3)n2)s1]

DIPEA (36 UL, 0.21 mmol, 3.0 eq) was added to a solution of N-(3-hydroxy-2,6-dimethyl-phenyl)-2-[1-[(3R)-pyrrolidin-3-yl]pyrazol-3-yl]amino]thiazole-5-carboxamide hydrochloride (BAA-056)(30 mg, 0.069 mmol, 1.0 eq) and benzyl isocyanate (11 μL, 0.083 mmol, 1.2 eq) in anhydrous DCM (1.2 mL) and the mixture stirred at RT, under nitrogen, for 18 h, before purifying the reaction mixture directly using normal phase chromatography (SiO₂) eluting with 0-30% MeOH: DCM then again with 5-20% MeOH: DCM to afford the title compound (4 mg, 0.019 mmol, 11%) as a white solid. MS (ES+) m/z 532.3 (M+H). ¹H NMR (Methanol-d₄, 300 MHz) δ 7.93 (s, 1H), 7.53 (d, 1H, J=2.0 Hz), 7.35-7.18 (m, 5H), 6.91 (d, 1H, J=8.2 Hz), 6.68 (d, 1H, J=8.2 Hz), 6.31 (s, 1H), 5.16-5.12 (m, 1H), 4.37 (s, 2H), 3.86-3.65 (m, 3H), 3.55-3.46 (m, 1H), 2.48-2.34 (m, 2H), 2.14 (s, 3H), 2.09 (s, 3H) ppm.

### 2-[[1-[(3R)-1-(Ethylcarbamoyl)pyrrolidin-3-yl]pyrazol-3-yl]amino]-N-(3-hydroxy-2,6-dimethyl-phenyl)thiazole-5-carboxamide (BAA-109)

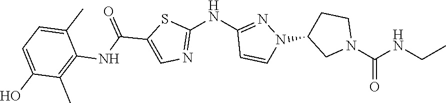 [SMILES: CCNC(=O)N1CC[C@@H](n2ccc(Nc3ncc(C(=O)Nc4c(C)ccc(O)c4C)s3)n2)C1]

Ethyl isocyanate (6 μL, 0.076 mmol, 1.1 eq) was added to a solution of N-(3-hydroxy-2,6-dimethyl-phenyl)-2-[1-[(3R)-pyrrolidin-3-yl]pyrazol-3-yl]amino]thiazole-5-carboxamide hydrochloride (BAA-056)(30 mg, 0.069 mmol, 1.0 eq) and DIPEA (42 μL, 0.24 mmol, 3.5 eq) in anhydrous MeCN (1.2 mL) and the mixture warmed to 40° C., under nitrogen, for 18 h, before purifying the reaction mixture directly using normal phase chromatography (SiO₂) eluting with 0-50% MeOH: DCM followed by a second purification with 0-50% MeOH: EtOAC to afford the title compound (5.5 mg, 0.011 mmol, 17%) as an off-white solid. MS (ES+) m/z 470.3 (M+H). ¹H NMR (Methanol-d₄, 300 MHz) δ 7.94 (s, 1H), 7.52 (d, 1H, J=2.0 Hz), 6.91 (d, 1H, J=8.2 Hz), 6.68 (d, 1H, J=8.2 Hz), 6.31 (s, 1H), 5.16-5.08 (m, 1H), 3.81-3.59 (m, 3H), 3.50-3.41 (m, 1H), 3.20 (q, 2H, J=7.2 Hz), 2.49-2.30 (m, 2H), 2.14 (s, 3H), 2.09 (s, 3H), 1.12 (t, 3H, J=7.2 Hz) ppm.

### 2-[[1-[(3R)-1-(Cyclopropylcarbamoyl)pyrrolidin-3-yl]pyrazol-3-yl]amino]-N-(3-hydroxy-2.6-dimethyl-phenyl)thiazole-5-carboxamide (BAA-110)

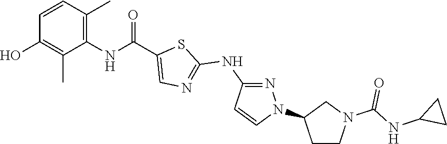 [SMILES: Cc1ccc(O)c(C)c1NC(=O)c1cnc(Nc2ccn([C@@H]3CCN(C(=O)NC4CC4)C3)n2)s1]

Prepared similarly using cyclopropyl isocyanate (6.5 L, 0.081 mmol, 1.0 eq) and DIPEA
(56 μL, 0.32 mmol, 4.0 eq) to afford the title compound (11 mg, 0.0228 mmol, 28%) as an off-white solid after normal phase chromatography (SiO₂) eluting with 0-35% MeOH: DCM then again with 0 -50% MeOH: EtOAC. MS (ES+) m/z 482.0 (M+H). ¹H NMR (Methanol-d₄, 300 MHz) δ 7.94 (s, 1H), 7.51 (d, 1H, J=2.1 Hz), 6.91 (d, 1H, J=8.3 Hz), 6.68 (d, 1H, J=8.2 Hz), 6.30 (s, 1H), 5.11 (p, 1H, J=6.3 Hz), 3.76 (dd, 1H, J=10.6, 7.3 Hz), 3.70-3.58 (m, 2H), 3.47-3.39 (m, 1H), 2.54 (tt, 1H, J=7.0, 3.7 Hz), 2.44-2.30 (m, 2H), 2.14 (s, 3H), 2.09 (s, 3H), 0.69-0.63 (m, 2H), 0.51-0.46 (m, 2H) ppm.
   Urea Formation from BAA-056 with Carbamoyl Chlorides

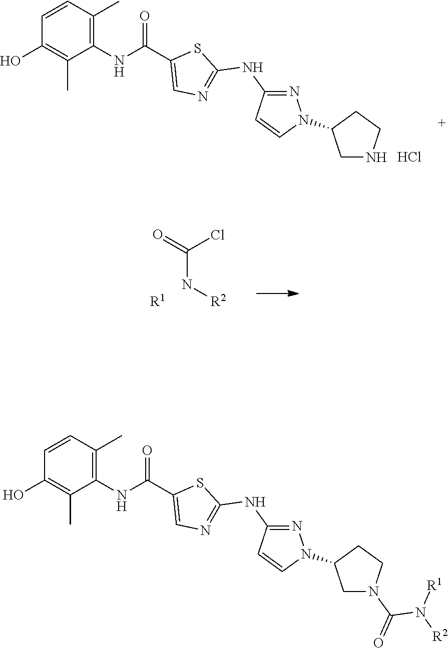

### 2-[[1-[(3R)-1-[Ethyl(methyl) carbamoyl]pyrrolidin-3-yl]pyrazol-3-yl]amino]-N-(3-hydroxy-2,6-dimethyl-phenyl)thiazole-5-carboxamide (BAA-111)

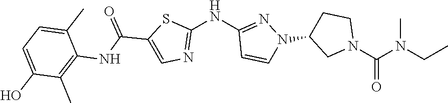 [SMILES: CCN(C)C(=O)N1CC[C@@H](n2ccc(Nc3ncc(C(=O)Nc4c(C)ccc(O)c4C)s3)n2)C1]

N-Ethyl-N-methylcarbamoyl Chloride (8.5 L, 0.081 mmol) was added to a solution of N-(3-hydroxy-2,6-dimethyl-phenyl)-2-[1-[(3R)-pyrrolidin-3-yl]pyrazol-3-yl]amino]thiazole-5-carboxamide hydrochloride (BAA-056)(35 mg, 0.081 mmol) and DIPEA (56 UL, 0.32 mmol) in anhydrous acetonitrile (1.0 mL) and the reaction mixture was warmed to 40° C., with stirring under nitrogen overnight, before purifying the reaction mixture directly using flash column chromatography over silica, eluting with 0-25% methanol/DCM, then again, eluting with 0-40% methanol/ethyl acetate to give the product (22 mg, 57% yield) as a white solid. MS (ES+) m/z 484.1 (M+H). ¹H NMR (Methanol-d₄, 300 MHz) δ 7.94 (s, 1H), 7.54 (d, 1H, J=2.1 Hz), 6.91 (d, 1H, J=8.2 Hz), 6.68 (d, 1H, J=8.2 Hz), 6.32 (d, 1H, J=2.0 Hz), 5.06 (p, 1H, J=6.6 Hz), 3.81 (dd, 1H, J=10.7, 7.1 Hz), 3.72-3.64 (m, 2H), 3.59-3.51 (m, 1H), 3.25 (q, 2H, J=7.1 Hz), 2.85 (s, 3H), 2.44-2.24 (m, 2H), 2.14 (s, 3H), 2.09 (s, 3H), 1.15 (t, 3H, J=7.1 Hz).

### 2-[[1-[(3R)-1-(Diethylcarbamoyl)pyrrolidin-3-yl]pyrazol-3-yl]amino]-N-(3-hydroxy-2,6-dimethyl-phenyl)thiazole-5-carboxamide (BAA-112)

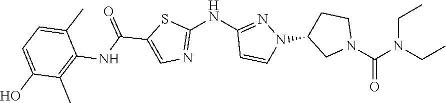 [SMILES: CCN(CC)C(=O)N1CC[C@@H](n2ccc(Nc3ncc(C(=O)Nc4c(C)ccc(O)c4C)s3)n2)C1]

Prepared similarly using diethylcarbamoyl chloride (10 μL, 0.081 mmol) to give the product
(24 mg, 60% yield) as an off-white solid after flash column chromatography over silica, eluting with 0-25% methanol/DCM, followed by a second purification eluting with 0-40% methanol/ethyl acetate. MS (ES+) m/z 498.1 (M+H). ¹H NMR (Methanol-d₄, 300 MHz) δ 7.94 (s, 1H), 7.53 (d, 1H, J=2.0 Hz), 6.91 (d, 1H, J=8.2 Hz), 6.68 (d, 1H, J=8.2 Hz), 6.32 (d, 1H, J=1.8 Hz), 5.05 (p, 1H, J=6.5 Hz), 3.80 (dd, 1H, J=10.7, 7.1 Hz), 3.72-3.63 (m, 2H), 3.58-3.50 (m, 1H), 3.25 (q, 4H, J=7.2 Hz), 2.45-2.24 (m, 2H), 2.14 (s, 3H), 2.09 (s, 3H), 1.14 (t, 6H, J=7.1 Hz) ppm.

### Alkylation of BAA-056

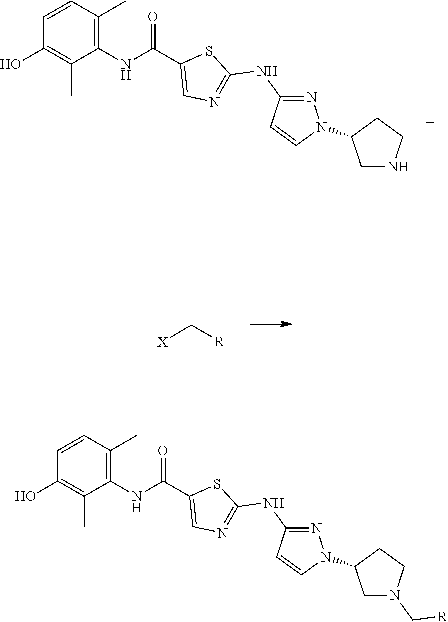

### 2-[[1-[(3R)-1-Benzylpyrrolidin-3-yl]pyrazol-3-yl]amino]-N-(3-hydroxy-2,6-dimethyl-phenyl)thiazole-5-carboxamide (BAA-113)

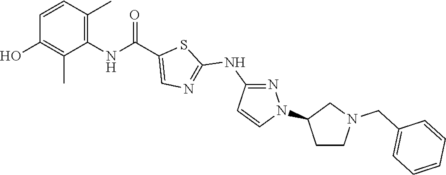 [SMILES: Cc1ccc(O)c(C)c1NC(=O)c1cnc(Nc2ccn([C@@H]3CCN(Cc4ccccc4)C3)n2)s1]

Benzyl bromide (10 μL, 0.085 mmol, 1.05 eq) was added to a stirred solution of N-(3-hydroxy-2,6-dimethyl-phenyl)-2-[1-[(3R)-pyrrolidin-3-yl]pyrazol-3-yl]amino]thiazole-5-carboxamide hydrochloride (BAA-056)(35 mg, 0.081 mmol, 1.0 eq) and DIPEA (42 μL, 0.24 mmol, 3.0 eq) in anhydrous MeCN (1.5 mL) and the mixture warmed to 40° C., under nitrogen, for 18 h, before purifying the reaction mixture directly using normal phase chromatography (SiO₂) eluting 0-30% MeOH: DCM, then again with 60-100% EtOAc: PE followed by 0-20% MeOH: EtOAc to afford the title compound (11 mg, 0.023 mmol, 28%) as an off-white solid. MS (ES+) m/z 489.0 (M+H). ¹H NMR (Methanol-d₄, 300 MHz) δ 8.03 (s, 1H), 7.48-7.45 (m, 1H), 7.44 (d, 1H, J=2.1 Hz), 7.38-7.25 (m, 4H), 6.92 (d, 1H, J=8.2 Hz), 6.69 (d, 1H, J=8.2 Hz), 6.35 (d, 1H, J=2.1 Hz), 5.16-5.07 (m, 1H), 3.99 (d, 1H, J=12.5 Hz), 3.73 (d, 1H, J=12.5 Hz), 3.22 (td, 1H, J=8.9, 4.7 Hz), 2.98-2.89 (m, 2H), 2.70 (q, 1H, J=8.4 Hz), 2.56-2.44 (m, 1H), 2.16 (s, 3H), 2.11 (s, 3H), 2.12-2.07 (m, 1H) ppm.

### N-(3-Hydroxy-2,6-dimethyl-phenyl)-2-[[1-[(3R)-1-propylpyrrolidin-3-yl]pyrazol-3-yl]amino]thiazole-5-carboxamide (BAA-114)

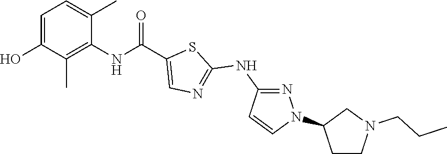 [SMILES: CCCN1CC[C@@H](n2ccc(Nc3ncc(C(=O)Nc4c(C)ccc(O)c4C)s3)n2)C1]

Prepared similarly using 1-bromopropane (8 μL, 0.089 mmol, 1.1 eq) and DIPEA (3.5 eq) to afford the title compound (9 mg, 0.020 mmol, 25%) as an off-white solid after normal phase chromatography (SiO₂) eluting with 0-30% MeOH: DCM, followed by a second purification with 0-40% MeOH: EtOAc. MS (ES+) m/z 441.1 (M+H). ¹H NMR (Methanol-d₄, 300 MHz) δ 8.01 (s, 1H), 7.50 (d, 1H, J=2.1 Hz), 6.93 (d, 1H, J=8.2 Hz), 6.71 (d, 1H, J=8.2 Hz), 6.37 (d, 1H, J=2.1 Hz), 5.28-5.20 (m, 1H), 3.50-3.38 (m, 2H), 3.24-3.18 (m, 1H), 2.99-2.79 (m, 3H), 2.64-2.51 (m, 1H), 2.25-2.20 (m, 1H), 2.17 (s, 3H), 2.12 (s, 3H), 1.82-1.69 (m, 2H), 1.03 (t, 3H, J=7.4 Hz) ppm.

### N-(3-Hydroxy-2,6-dimethyl-phenyl)-2-[[1-[(3R)-1-(2-phenylethyl)pyrrolidin-3-yl]pyrazol-3-yl]amino thiazole-5-carboxamide (BAA-115)

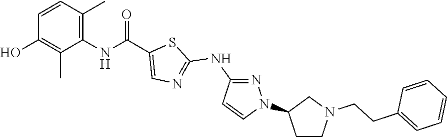 [SMILES: Cc1ccc(O)c(C)c1NC(=O)c1cnc(Nc2ccn([C@@H]3CCN(CCc4ccccc4)C3)n2)s1]

Prepared similarly using (2-bromoethyl)benzene (12 μL, 0.089 mmol, 1.1 eq) and DIPEA
(49 μL, 0.28 mmol, 3.5 eq) to afford the title compound (6 mg, 0.012 mmol, 15%) as an off-white solid after normal phase chromatography (SiO₂) eluting with 0-30% MeOH: DCM, then again with 5-50% MeOH: EtOAc. MS (ES+) m/z 504.0 (M+H). ¹H NMR (Methanol-d₄, 300 MHz) δ 8.02 (s, 1H), 7.45 (d, 1H, J=2.1 Hz), 7.28-7.26 (m, 4H), 7.22-7.15 (m, 1H), 6.92 (d, 1H, J=8.2 Hz), 6.69 (d, 1H, J=8.2 Hz), 6.38 (d, 1H, J=2.1 Hz), 5.20-5.12 (m, 1H), 3.39-3.34 (m, 1H), 3.29-3.24 (m, 2H), 3.08-2.90 (m, 5H), 2.78-2.69 (m, 1H), 2.60-2.47 (m, 1H), 2.16 (s, 3H), 2.11 (s, 3H) ppm.

### N-(3-Hydroxy-2,6-dimethyl-phenyl)-2-[[1-[(3R)-1-methylpyrrolidin-3-yl]pyrazol-3-yl]amino]thiazole-5-carboxamide (BAA-116)

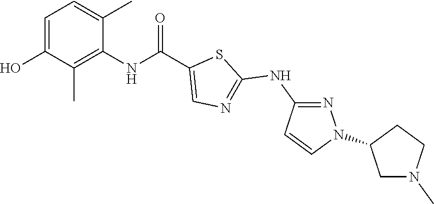 [SMILES: Cc1ccc(O)c(C)c1NC(=O)c1cnc(Nc2ccn([C@@H]3CCN(C)C3)n2)s1]

Prepared similarly using iodomethane (7 μL, 0.12 mmol, 1.2 eq) and K₂CO₃ (48 mg, 0.34 mmol, 3.0 eq) at RT to afford the title compound (5 mg, 0.012 mmol, 6%) as a white solid after normal phase chromatography (SiO₂) eluting with 0-40% MeOH: DCM then again with 0-50% MeOH: EtOAc. MS (ES+) m/z 413.1 (M+H). ¹H NMR (Methanol-d₄, 300 MHz) δ 8.00 (s, 1H), 7.45 (d, 1H, J=2.1 Hz), 6.91 (d, 1H, J=8.2 Hz), 6.69 (d, 1H, J=8.2 Hz), 6.37 (d, 1H, J=2.1 Hz), 5.18-5.10 (m, 1H), 3.20 (td, 1H, J=9.0, 4.8 Hz), 3.10 (dd, 1H, J=10.8, 3.7 Hz), 2.96 (dd, 1H, J=10.7, 7.4 Hz), 2.70-2.62 (m, 1H), 2.57 (s, 3H), 2.55-2.46 (m, 1H), 2.18-2.13 (m, 1H), 2.15 (s, 3H), 2.10 (s, 3H) ppm.

### Syntheses Using Methoxy Protected Phenol

### Pd coupling of 2-bromo-N-(3-methoxy-2,6-dimethyl-phenyl)thiazole-5-carboxamide with substituted amino pyrazoles

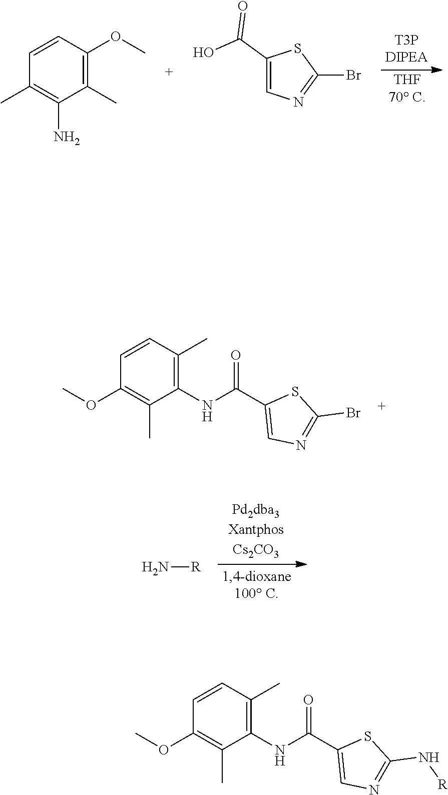

### 2-Bromo-N-(3-methoxy-2,6-dimethyl-phenyl)thiazole-5-carboxamide

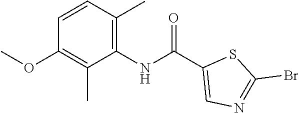 [SMILES: COc1ccc(C)c(NC(=O)c2cnc(Br)s2)c1C]

To a mixture of 2-bromo-1,3-thiazole-5-carboxylic acid (1.0 g, 4.81 mmol, 1.0 eq) and 3-methoxy-2,6-dimethyl-aniline (800 mg, 5.29 mmol, 1.1 eq), (prepared as reported in Can. J. Chem. 90:75-84 (2012) in THF (40 mL) under nitrogen, was added DIPEA (1.7 mL, 9.61 mmol, 2.0 eq) and T3P (50% in EtOAc, 4.2 mL, 7.21 mmol, 1.5 eq) and the reaction was heated at 70° C. for 18 h. Then, the crude was concentrated and partitioned between EtOAc and water. The aqueous phase was extracted with EtOAc (x 2) and the combined organic layers were dried (MgSO₄), filtered and concentrated in vacuo. The crude material was chromatographed (SiO₂) eluting with 0-100% EtOAc: PE to afford the title compound (1.45 g, 4.25 mmol, 88%) as a white solid. MS (ES-) m/z 339.0/341.0 (M−H), Br-isotope pattern. ¹H NMR (300 MHz, DMSO-d₆) δ 10.15 (s, 1H), 8.44 (s, 1H), 7.10 (d, J=8.4 Hz, 1H), 6.88 (d, J=8.4 Hz, 1H), 3.79 (s, 3H), 2.11 (s, 3H), 2.01 (s, 3H).

General Method I: Pd Coupling of 2-Bromo-N-(3-Methoxy-2,6-Dimethyl-Phenyl)Thiazole-5-Carboxamide and Other 2-Bromo-N-(3-Methoxy-2,6-Disubstituted-Phenyl)Thiazole-5-Carboxamide with Substituted Amino Pyrazoles:

To a mixture of 2-bromo-N-(3-methoxy-2,6-dimethyl-phenyl)thiazole-5-carboxamide (or other 2-bromo-N-(3-methoxy-2,6-disubstituted-phenyl)thiazole-5-carboxamide)(1.0 eq) and amino pyrazole (2.5-3.0 eq) dry dioxane or in 1,4-dioxane and water (9:1, 0.03-0.09 M), evacuated and purged with nitrogen, was added Cs₂CO₃ (3.0 eq) or K₂CO₃ (4 eq), Pd₂dba₃ (0.15 eq) or Pd(OAc) 2
(0.1 eq) and XantPhos (0.25 eq) and heated to 100° C. for 18 h. The reaction was monitored by consumption of starting material via UPLC. The mixture was filtered through a pad of dicalite, washed with EtOAc: MeOH and the filtrate concentrated in vacuo. The residue was partitioned between EtOAc and water, the aqueous phase extracted with EtOAc (x 3), the combined organics dried (MgSO₄), filtered and concentrated in vacuo. The crude material was purified by either normal phase chromatography (SiO₂) using a gradient of EtOAc: PE.

### 2-[[1-[2-(Dimethylamino)-2-oxo-ethyl]pyrazol-3-yl]amino]-N-(3-methoxy-2,6-dimethyl-phenyl)thiazole-5-carboxamide

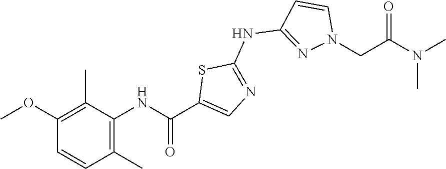

Prepared as described from 2-bromo-N-(3-methoxy-2,6-dimethyl-phenyl)thiazole-5-carboxamide (60 mg, 0.176 mmol, 1.0 eq), 2-(3-aminopyrazol-1-yl)-N,N-dimethyl-acetamide (89 mg, 0.528 mmol, 3.0 eq), Cs₂CO₃ (173 mg, 0.528 mmol, 3.0 eq), Pd₂dba₃ (24 mg, 0.0264 mmol, 0.15 eq) and XantPhos (25 mg, 0.0440 mmol, 0.25 eq) in 1,4-dioxane (4.5 mL) and water (0.5 mL) to afford the title compound (40 mg, 0.0933 mmol, 53%) as a white solid after normal phase chromatography (SiO₂) eluting with 0-20% MeOH: DCM. MS (ES+) m/z 428.9 (M+H). ¹H NMR
(300 MHz, DMSO-d₆) δ 11.07 (s, 1H), 9.59 (s, 1H), 8.11 (s, 1H), 7.58 (d, J=2.4 Hz, 1H), 7.07 (d, J=8.4 Hz, 1H), 6.85 (d, J=8.4 Hz, 1H), 5.98 (d, J=2.3 Hz, 1H), 5.04 (s, 2H), 3.78 (s, 3H), 3.05 (s, 3H), 2.85 (s, 3H), 2.12 (s, 3H), 2.02 (s, 3H).

The following example compounds were prepared similarly using Method I using the appropriately substituted amine.

| Structure & Name | Amine | Analytical Data |
|---|---|---|
| 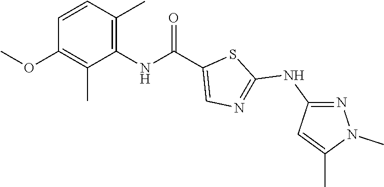 | 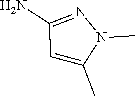 | MS (ES+) m/z 372.0 (M + H). ¹H NMR (300 MHz, DMSO-d₆) δ 10.94 (s, 1H), 9.56 (s, 1H), 8.09 (s, 1H), 7.07 (d, J = 8.4 Hz, 1H), 6.84 (d, J = 8.4 Hz, 1H), 5.78 (d, J = 0.8 Hz, 1H), 3.78 (s, 3H), 3.67 (s, 3H), |
| | | 2.23 (s, 3H), 2.12 |
| 2-[(1,5-dimethylpyrazol-3- | | (s, 3H), 2.02 (s, |
| yl)amino]-N-(3-methoxy-2,6- | | 3H). |
| dimethyl-phenyl)thiazole-5- | | |
| carboxamide | | |
| 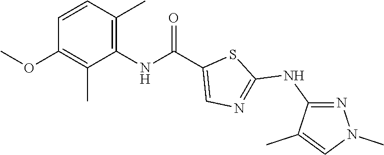 | 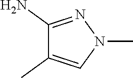 | MS (ES+) m/z 372.0 (M + H). ¹H NMR (300 MHz, DMSO-d₆) δ 10.69 (s, 1H), 9.54 (s, 1H), 8.11 (s, 1H), 7.43 (d, J = 0.9 Hz, 1H), 7.07 (d, J = 8.4 Hz, 1H), 6.84 (d, J = 8.4 |
| | | Hz, 1H), 3.78 (s, |
| 2-[(1,4-dimethylpyrazol-3- | | 3H), 3.74 (s, 3H), |
| yl)amino]-N-(3-methoxy-2,6- | | 2.12 (s, 3H), 2.02 |
| dimethyl-phenyl)thiazole-5- | | (s, 3H), 1.99 (s, 3H). |
| carboxamide | | |
| 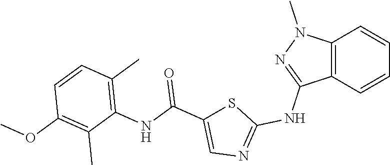 [SMILES: COc1ccc(C)c(NC(=O)c2cnc(Nc3nn(C)c4ccccc34)s2)c1C] | 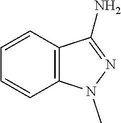 | MS (ES+) m/z 408.3 (M + H). ¹H NMR (300 MHz, DMSO-d₆) δ 11.88 (s, 1H), 9.63 (s, 1H), 8.23 (s, 1H), 8.11 (d, J = 8.2 Hz, 1H), 7.56 (d, J = 8.6 Hz, 1H), 7.44 (ddd, J = 8.4, 6.8, 1.1 Hz, |
| | | 1H), 7.16-7.04 (m, |
| N-(3-methoxy-2,6-dimethyl- | | 2H), 6.86 (d, J = 8.4 |
| phenyl)-2-[(1-methylindazol-3- | | Hz, 1H), 3.99 |
| yl)amino]thiazole-5-carboxamide | | (s, 3H), 3.79 (s, 3H), |
| | | 2.15 (s, 3H), |
| | | 2.05 (s, 3H). |
| 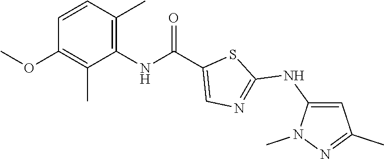 | 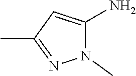 | MS (ES+) m/z 372.3 (M + H). ¹H NMR (300 MHz, DMSO-d₆) δ 10.66-10.26 (brs, 1H), 9.62 (s, 1H), 8.05 (s, 1H), 7.07 (d, J = 8.4 Hz, 1H), 6.85 (d, J = 8.4 Hz, 1H), 6.23-6.03 (brs, 1H), |
| | | 3.78 (s, 3H), |
| 2-[(2,5-dimethylpyrazol-3- | | 3.63 (s, 3H), 2.12 |
| yl)amino]-N-(3-methoxy-2,6- | | (s, 3H), 2.11 (s, |
| dimethyl-phenyl)thiazole-5- | | 3H), 2.01 (s, 3H). |
| carboxamide | | |
| 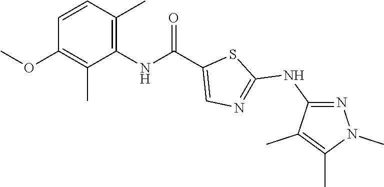 | 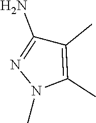 | MS (ES+) m/z 386.3 (M + H). ¹H NMR (300 MHz, DMSO-d₆) δ 10.57 (s, 1H), 9.51 (s, 1H), 8.09 (s, 1H), 7.06 (d, J = 8.3 Hz, 1H), 6.84 (d, J = 8.4 Hz, 1H), 3.78 (s, 3H), 3.66 (s, 3H), 2.15 (s, 3H), 2.12 (s, 3H), 2.02 (s, 3H), 1.92 (s, 3H). |
| N-(3-methoxy-2,6-dimethyl- | | |
| phenyl)-2-[(1,4,5- | | |
| trimethylpyrazol-3- | | |
| yl)amino]thiazole-5-carboxamide | | |
| 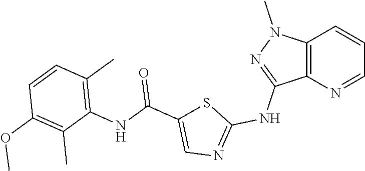 [SMILES: COc1ccc(C)c(NC(=O)c2cnc(Nc3nn(C)c4cccnc34)s2)c1C] | 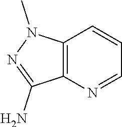 | Used in the subsequent reaction without isolation |
| N-(3-methoxy-2,6- | | |
| dimethylphenyl)-2-((1-methyl- | | |
| 1H-pyrazolo[4,3-b]pyridin-3- | | |
| yl)amino)thiazole-5- | | |
| carboxamide | | |
| 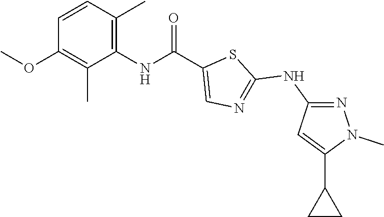 | 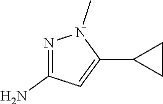 | MS (ES+) m/z 398.3 (M + H). ¹H NMR (300 MHz, DMSO-d6) δ 10.42 (s, 1H), 9.62 (s, 1H), 8.05 (s, 1H), 7.07 (d, J = 8.4 Hz, 1H), 6.85 (d, J = 8.4 Hz, 1H), 6.08 (s, 1H), 3.78 (s, 3H), 3.61 (s, 3H), 2.11 (s, 3H), 2.01 (s, 3H), 1.87-1.74 (m, |
| | | 1H), 0.88-0.76 |
| 2-((5-cyclopropyl-1-methyl-1H- | | (m, 2H), 0.69- |
| pyrazol-3-yl)amino)-N-(3- | | 0.58 (m, 2H). |
| methoxy-2,6- | | |
| dimethylphenyl) thiazole-5- | | |
| carboxamide | | |
| 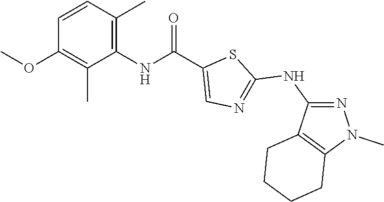 | 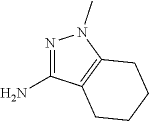 | MS (ES+) m/z 412.3 (M + H). ¹H NMR (300 MHz, DMSO-d6) δ 10.01 (s, 1H), 9.59 (s, 1H), 8.00 (s, 1H), 7.06 (d, J = 8.4 Hz, 1H), 6.84 (d, J = 8.5 Hz, 1H), 3.78 (s, 3H), 3.59 (s, 3H), 2.36- 2.24 (m, 2H), 2.10 (s, 3H), 2.00 (s, 3H), 1.79-1.56 (m, 4H). |
| N-(3-methoxy-2,6- | | |
| dimethylphenyl)-2-((1-methyl- | | |
| 4,5,6,7-tetrahydro-1H-indazol-3- | | |
| yl)amino)thiazole-5- | | |
| carboxamide | | |
| 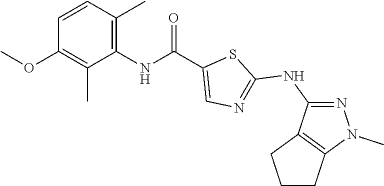 | 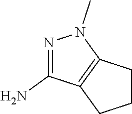 | MS (ES+) m/z 398.3 (M + H). |
| N-(3-methoxy-2,6- | | |
| dimethylphenyl)-2-((1-methyl- | | |
| 1,4,5,6- | | |
| tetrahydrocyclopenta[c]pyrazol- | | |
| 3-yl)amino)thiazole-5- | | |
| carboxamide | | |
| 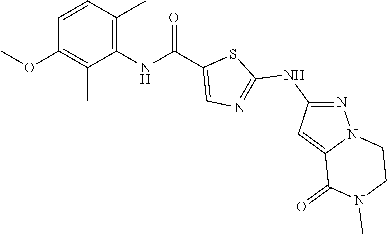 [SMILES: COc1ccc(C)c(NC(=O)c2cnc(Nc3cc4n(n3)CCN(C)C4=O)s2)c1C] | 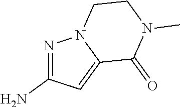 | MS (ES+) m/z 427.1 (M + H). |
| N-(3-methoxy-2,6- | | |
| dimethylphenyl)-2-((5-methyl-4- | | |
| oxo-4,5,6,7- | | |
| tetrahydropyrazolo[1,5- | | |
| a]pyrazin-2-yl)amino)thiazole-5- | | |
| carboxamide | | |
| 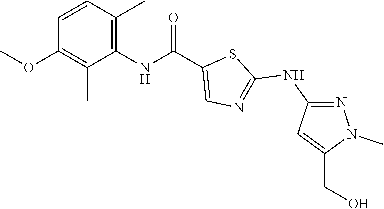 | 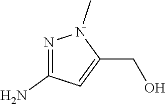 | MS (ES+) m/z 388.0 (M + H). |
| 2-((5-(hydroxymethyl)-1-methyl- | | |
| 1H-pyrazol-3-yl)amino)-N-(3- | | |
| methoxy-2,6- | | |
| dimethylphenyl)thiazole-5- | | |
| carboxamide | | |
| 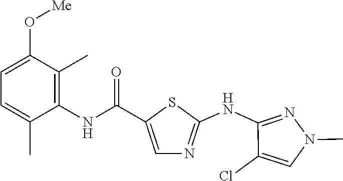 [SMILES: COc1ccc(C)c(NC(=O)c2cnc(Nc3nn(C)cc3Cl)s2)c1C] | 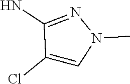 | MS (ES+) m/z = 392.1, 394.1 [M + H]+ (mono Cl pattern). ¹H NMR (400 MHz, DMSO- d6) δ 10.91 (s, 1H), 9.57 (s, 1H), 8.09 (s, 1H), 7.92 (s, 1H), 7.06 (d, J = 8.4 Hz, 1H), 6.84 (d, J = 8.4 Hz, 1H), 3.79 (s, |
| | | 3H), 3.77 (s, 3H), |
| 2-((4-chloro-1-methyl-1H- | | 2.11 (s, 3H), 2.01 |
| pyrazol-3-yl)amino)-N-(3- | | (s, 3H). |
| methoxy-2,6- | | |
| dimethylphenyl)thiazole-5- | | |
| carboxamide | | |
| 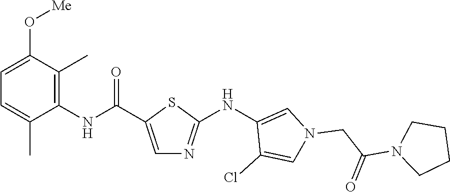 [SMILES: COc1ccc(C)c(NC(=O)c2cnc(Nc3cn(CC(=O)N4CCCC4)cc3Cl)s2)c1C] | 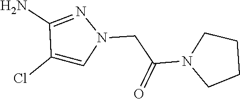 [SMILES: Nc1nn(CC(=O)N2CCCC2)cc1Cl] | MS (ES+) m/z = 489.2, 491.1 [M + H]+ (mono Cl pattern). ¹H NMR (400 MHz, DMSO-d6) δ 8.63 (s, 1H), 7.85 (s, 1H), 7.54 (s, 1H), 6.99 (d, J = 8.3 Hz, 1H), 6.76 (d, J = 8.4 Hz, 1H), 4.76 (s, 2H), 3.81-3.72 (m, 5H), 3.56 (t, J = 6.8 Hz, 2H), |
| | | 2.10 (s, 3H), 1.99 |
| 2-((4-chloro-1-(2-oxo-2- | | (s, 3H), 1.94- |
| (pyrrolidin-1-yl)ethyl)-1H- | | 1.84 (m, 2H), 1.83- |
| pyrazol-3-yl)amino)-N-(3- | | 1.70 (m, 2H). |
| methoxy-2,6- | | |
| dimethylphenyl)thiazole-5- | | |
| carboxamide | | |
| 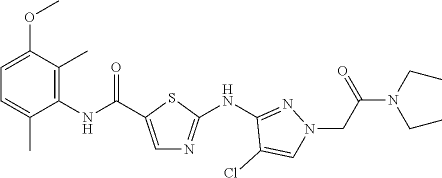 [SMILES: CCN(CC)C(=O)Cn1cc(Cl)c(Nc2ncc(C(=O)Nc3c(C)ccc(OC)c3C)s2)n1] | 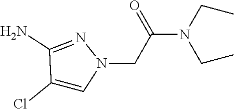 | MS (ES+) m/z = 491.1/493.0 [M + H]+ mono Cl pattern. ¹H NMR (400 MHz, CHLOROFORM-D) δ 7.89 (s, 1H), 7.53 (d, J = 15.2 Hz, 1H), 7.16- 7.00 (m, 2H), 6.75 (d, J = 8.5 Hz, 1H), 4.85 (s, 2H), 3.81 (s, 3H), 3.43 |
| | | (dq, J = 21.0, 7.1 |
| 2-((4-chloro-1-(2-(diethylamino)- | | Hz, 4H), 2.22 (s, |
| 2-oxoethyl)-1H-pyrazol-3- | | 3H), 2.15 (s, 3H), |
| yl)amino)-N-(3-methoxy-2,6- | | 1.26 (t, J = 7.2 Hz, |
| dimethylphenyl)thiazole-5- | | 3H), 1.13 (q, J = 7.0 |
| carboxamide | | Hz, 3H). |
| 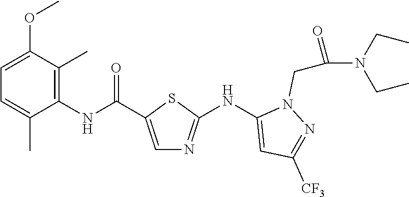 | 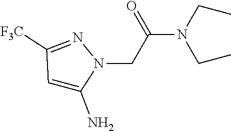 | MS (ES+) m/z = 511.2 [M + H]+. ¹H NMR (400 MHz, DMSO-D6) δ 9.15 (s, 1H), 8.02 (s, 1H), 6.85 (d, J = 8.4 Hz, 1H), 6.65 (d, J = 8.2 Hz, 1H), 5.07 (s, 2H), 3.35 (q, J = 7.0 Hz, 2H), 3.25 (q, J = 7.2 Hz, 2H), 2.02 (s, 3H), 1.94 (s, 3H), |
| | | 1.14 (t, J = 6.8 |
| 2-((1-(2-(diethylamino)-2- | | Hz, 3H), 1.00 (t, J = 7.0 |
| oxoethyl)-3-(trifluoromethyl)-1H- | | Hz, 3H). |
| pyrazol-5-yl) amino)-N-(3- | | |
| methoxy-2,6-dimethylphenyl) | | |
| thiazole-5-carboxamide | | |
| 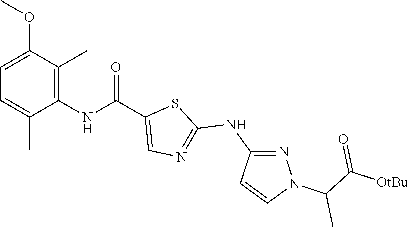 [SMILES: COc1ccc(C)c(NC(=O)c2cnc(Nc3ccn(C(C)C(=O)OC(C)(C)C)n3)s2)c1C] | 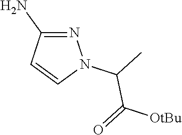 | MS (ES−) m/z = 470.3 [M − H]− |
| tert-butyl 2-(3-((5-((3-methoxy- | | |
| 2,6-dimethylphenyl) | | |
| carbamoyl)thiazol-2-yl)amino)- | | |
| 1H-pyrazol-1-yl)propanoate | | |
| 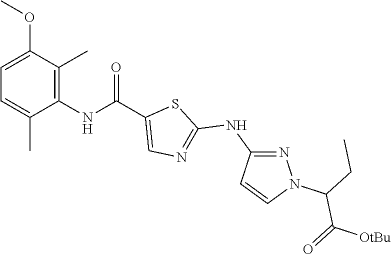 [SMILES: CCC(C(=O)OC(C)(C)C)n1ccc(Nc2ncc(C(=O)Nc3c(C)ccc(OC)c3C)s2)n1] | 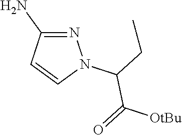 | MS (ES+) m/z = 486.2 [M + H]+ |
| tert-butyl 2-(3-((5-((3-methoxy- | | |
| 2,6-dimethylphenyl)carbamoyl) | | |
| thiazol-2-yl)amino)-1H-pyrazol- | | |
| 1-yl)butanoate | | |
| 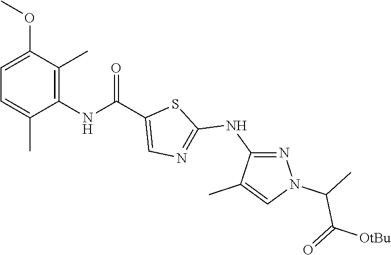 [SMILES: COc1ccc(C)c(NC(=O)c2cnc(Nc3nn(C(C)C(=O)OC(C)(C)C)cc3C)s2)c1C] | 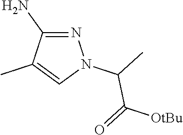 | MS (ES+) m/z = 486.4 [M + H]+; ¹H NMR (400 MHz, DMSO-D6) δ 10.74 (s, 1H), 9.53 (s, 1H), 8.11 (s, 1H), 7.54 (d, J = 1.0 Hz, 1H), 7.06 (d, J = 8.3 Hz, 1H), 6.84 (d, J = 8.4 Hz, 1H), 4.92 (q, J = 7.2 Hz, 1H), 3.77 (s, 3H), 2.11 (s, 3H), 2.01 (d, J = 1.9 Hz, 6H), 1.61 (d, J = 7.2 Hz, 3H), 1.37 (s, 9H). |
| tert-butyl 2-(3-((5-((3-methoxy- | | |
| 2,6-dimethylphenyl)carbamoyl) | | |
| thiazol-2-yl)amino)-4-methyl-1H- | | |
| pyrazol-1-yl)propanoate | | |
| 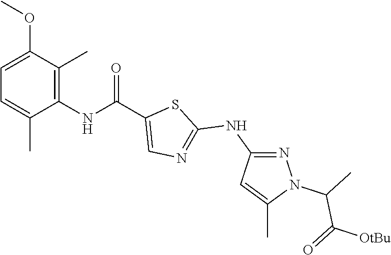 [SMILES: COc1ccc(C)c(NC(=O)c2cnc(Nc3cc(C)n(C(C)C(=O)OC(C)(C)C)n3)s2)c1C] | 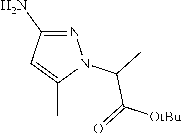 | MS (ES+) m/z = 486.2 [M + H]+ |
| tert-butyl 2-(3-((5-((3-methoxy- | | |
| 2,6-dimethylphenyl)carbamoyl) | | |
| thiazol-2-yl)amino)-5-methyl-1H- | | |
| pyrazol-1-yl)propanoate | | |
| 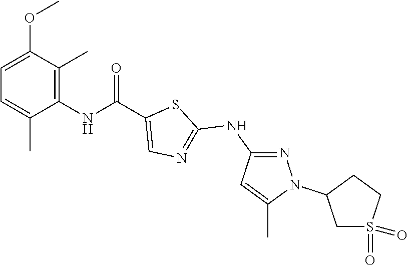 [SMILES: COc1ccc(C)c(NC(=O)c2cnc(Nc3cc(C)n(C4CCS(=O)(=O)C4)n3)s2)c1C] | 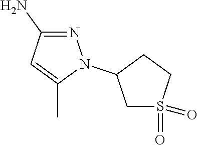 | MS (ES+) m/z = 476.2 [M + H]+ |
| 2-((1-(1,1-dioxidotetrahydro | | |
| thiophen-3-yl)-5-methyl-1H- | | |
| pyrazol-3-yl)amino)-N-(3- | | |
| methoxy-2,6-dimethylphenyl) | | |
| thiazole-5-carboxamide | | |
| 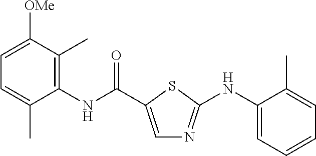 [SMILES: COc1ccc(C)c(NC(=O)c2cnc(Nc3ccccc3C)s2)c1C] | 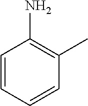 | MS (ES+) m/z = 368.1 [M + H]+; ¹H NMR (400 MHz, DMSO-d6) δ 9.41(s, 1H), 8.29 (s, 1H), 7.96 (s, 1H), 7.56 (d, J = 6.5 Hz, 1H), 7.49 (s, 1H), 7.23 (d, J = 7.6 Hz, 1H), 7.18 (d, J = 7.6 Hz, 1H), 7.07 (d, J = 9.7 |
| | | Hz, 1H), 6.85 (d, |
| N-(3-methoxy-2,6-dimethyl | | J = 8.5 Hz, 1H), |
| phenyl)-2-(o-tolylamino)thiazole- | | 3.77 (s, 3H), 2.23 |
| 5-carboxamide | | (s, 3H), 2.11 (s, |
| | | 3H), 2.01 (s, 3H). |
| 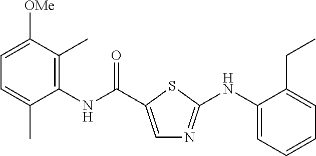 [SMILES: CCc1ccccc1Nc1ncc(C(=O)Nc2c(C)ccc(OC)c2C)s1] | 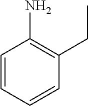 | MS (ES+) m/z = 382.2 [M + H]+; ¹H NMR (400 MHz, DMSO-d6) δ 9.51 (s, 1H), 8.29 (s, 1H), 7.99 (s, 1H), 7.60 (s, 1H), 7.57- 7.46 (m, 1H), 7.29 (d, J = 7.5 Hz, 1H), 7.23 (t, J = 7.3 Hz, 1H), 7.05 |
| | | (d, J = 8.6 Hz, 1H), |
| 2-((2-ethylphenyl)amino)-N-(3- | | 6.83 (d, J = 8.7 |
| methoxy-2,6-dimethylphenyl) | | Hz, 1H), 3.77 |
| thiazole-5-carboxamide | | (s, 3H), 2.65 (q, J = |
| | | 7.4 Hz, 2H), 2.09 |
| | | (s, 3H), 1.99 (s, 3H), 1.13 |
| | | (t, J = 7.5 Hz, 3H). |
| 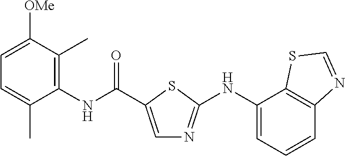 [SMILES: COc1ccc(C)c(NC(=O)c2cnc(Nc3cccc4ncsc34)s2)c1C] | 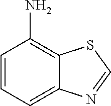 | MS (ES+) m/z = 411.1 [M + H]+. ¹H NMR (400 MHz, DMSO-d6) δ 9.16 (s, 1H), 8.86 (s, 1H), 7.88 (s, 1H), 7.78 (s, 1H), 7.45- 7.21 (m, 2H), 7.02 (d, J = 8.3 Hz, 1H), 6.79 (d, J = |
| | | 8.4 Hz, 1H), 3.76 |
| 2-(benzo[d]thiazol-7-ylamino)-N- | | (s, 3H), 2.11 (s, |
| (3-methoxy-2,6-dimethylphenyl) | | 3H), 2.00 (s, 3H). |
| thiazole-5-carboxamide | | |
| 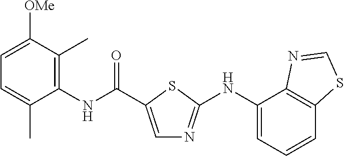 [SMILES: COc1ccc(C)c(NC(=O)c2cnc(Nc3cccc4scnc34)s2)c1C] | 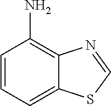 | MS (ES+) m/z = 411.1 [M + H]+. ¹H NMR (400 MHz, DMSO-d6) δ 11.01 (s, 1H), 9.70 (s, 1H), 9.41 (s, 1H), 8.56 (d, J = 7.9 Hz, 1H), 8.16 (s, 1H), 7.78 (d, J = 7.9 Hz, 1H), 7.48 (t, |
| | | J = 8.0 Hz, 1H), |
| 2-(benzo[d]thiazol-4-ylamino)-N- | | 7.07 (d, J = 8.4 Hz, |
| (3-methoxy-2,6-dimethylphenyl) | | 1H), 6.85 (d, J = |
| thiazole-5-carboxamide | | 8.4 Hz, 1H), 3.78 |
| | | (s, 3H), 2.13 (s, 3H), |
| | | 2.03 (s, 3H). |
| 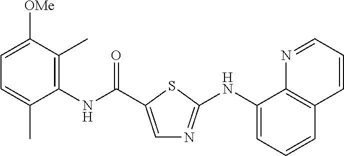 [SMILES: COc1ccc(C)c(NC(=O)c2cnc(Nc3cccc4cccnc34)s2)c1C] | 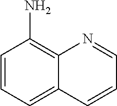 [SMILES: Nc1cccc2cccnc12] | MS (ES+) m/z = 405.2 [M + H]+. ¹H NMR (400 MHz, DMSO-d6) δ 8.95 (s, 1H), 8.82 (d, J = 5.5 Hz, 1H), 8.41 (d, J = 8.1 Hz, 1H), 8.20 (s, 1H), 7.72-7.54 (m, 3H), 7.08 (d, J = 8.4 Hz, 1H), |
| | | 6.86 (d, J = 8.3 Hz, |
| N-(3-methoxy-2,6-dimethyl | | 1H), 3.78 (s, 3H), 2.13 |
| phenyl)-2-(quinolin-8-ylamino) | | (s, 3H), 2.03 (s, 3H). |
| thiazole-5-carboxamide | | |
| 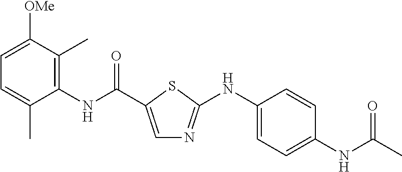 [SMILES: COc1ccc(C)c(NC(=O)c2cnc(Nc3ccc(NC(C)=O)cc3)s2)c1C] | 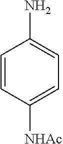 [SMILES: CC(=O)Nc1ccc(N)cc1] | MS (ES+) m/z = 411.2 [M + H]+. ¹H NMR (400 MHz, DMSO-d6) δ 10.49 (s, 1H), 9.87 (s, 1H), 9.60 (s, 1H), 8.06 (s, 1H), 7.52 (s, 4H), 7.06 (d, J = 8.3 Hz, 1H), 6.84 (d, J = 8.4 Hz, 1H), 3.78 (s, 3H), 2.11 (s, 3H), 2.02 (s, 3H), 2.01 (s, 3H). |
| 2-((4-acetamidophenyl)amino)- | | |
| N-(3-methoxy-2,6-dimethyl | | |
| phenyl)thiazole-5-carboxamide | | |
| 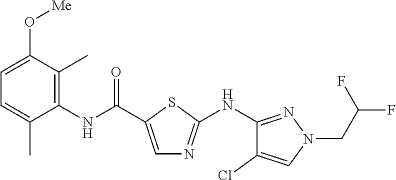 [SMILES: COc1ccc(C)c(NC(=O)c2cnc(Nc3nn(CC(F)F)cc3Cl)s2)c1C] | 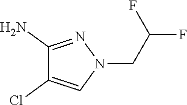 [SMILES: Nc1nn(CC(F)F)cc1Cl] | MS (ES+) m/z = 442.2/444.2 (mono Cl pattern) [M + H]+, ¹H NMR (400 MHz, DMSO-d6) δ 9.83 (s, 1H), 8.06 (s, 1H), 7.07 (d, J = 8.3 Hz, 1H), 6.85 (d, J = 8.2 Hz, 1H), 6.47-6.14 (m, 1H), 4.52-4.34 (m, 1H), 4.18- |
| | | 4.08 (m, 1H), 4.08 |
| 2-((4-chloro-1-(2,2- | | (s, 3H), 2.10 (s, |
| difluoroethyl)-1H-pyrazol-3- | | 3H), 2.00 (s, 3H). |
| yl)amino)-N-(3-methoxy-2,6- | | ¹⁹F NMR (376 |
| dimethylphenyl)thiazole-5- | | MHz, DMSO-d6) δ |
| carboxamide | | −122.39. |
| 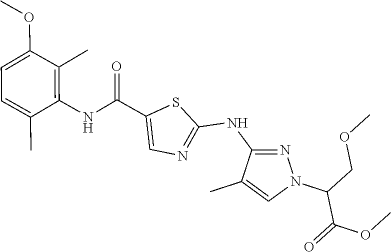 | 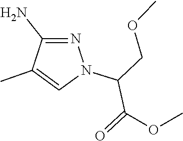 | MS (ES+) m/z = 474.2, [M + H]+; ¹H NMR (400 MHz, CDCl3) δ 8.44 (br s, 1H), 7.88 (s, 1H), 7.41 (s, 1H), 7.36 (br s, 1H), 7.12- 7.01 (m, 1H), 6.75 (d, J = 8.4 Hz, 1H), 5.07 (br m, 1H), 3.95 (dd, J = 10.2, 3.8 Hz, 1H), 3.82 (s, 3H), 3.78 (s, 3H), 3.38 (s, 3H), 2.22 (s, 3H), 2.15 (s, 3H). |
| methyl 3-methoxy-2-(3-((5-((3- | | |
| methoxy-2,6-dimethylphenyl) | | |
| carbamoyl)thiazol-2-yl)amino)-4- | | |
| methyl-1H-pyrazol-1- | | |
| yl)propanoate | | |
| 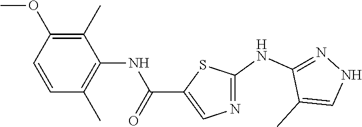 [SMILES: COc1ccc(C)c(NC(=O)c2cnc(Nc3n[nH]cc3C)s2)c1C] | 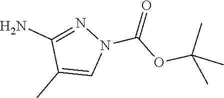 | MS (ES+) m/z 358 (M + H). ¹H NMR (300 MHz, MeOD) δ 8.01 (d, J = 11.3 Hz, 1H), 7.41 (s, 1H), 7.06 (d, J = 8.4 Hz, 1H), 6.82 (d, J = 8.4 |
| | | Hz, 1H), 3.81 (s, |
| N-(3-methoxy-2,6- | | 3H), 2.18 (s, 3H), |
| dimethylphenyl)-2-((4-methyl- | | 2.10 (s, 3H), 2.06 (s, 3H). |
| 1H-pyrazol-3-yl)amino)thiazole- | | |
| 5-carboxamide | | |
| 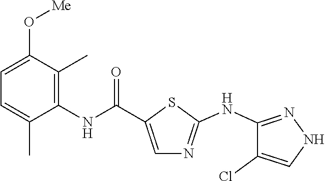 [SMILES: COc1ccc(C)c(NC(=O)c2cnc(Nc3n[nH]cc3Cl)s2)c1C] | 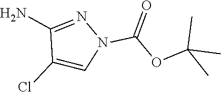 | MS (ES+) m/z = 378.1, 380.1 [M + H]+ (mono Cl pattern); ¹H NMR (400 MHz, DMSO- d6) δ 12.82 (s, 1H), 10.87 (s, 1H), 9.56 (s, 1H), 8.09 (s, 1H), 7.98 (s, 1H), 7.06 (d, J = 8.4 Hz, 1H), 6.84 (d, |
| | | J = 8.4 Hz, 1H), |
| 2-((4-chloro-1H-pyrazol-3- | | 3.77 (s, 3H), 2.11 |
| yl)amino)-N-(3-methoxy-2,6- | | (s, 3H), 2.01 (s, 3H). |
| dimethylphenyl)thiazole-5- | | |
| carboxamide | | |

### Ethyl 3-[[5-[(3-methoxy-2,6-dimethyl-phenyl) carbamoyl]thiazol-2-yl]amino]-1-methyl-pyrazole-4-carboxylate

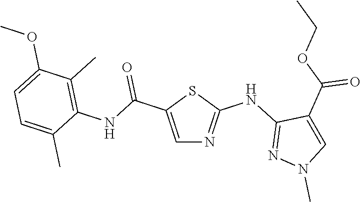 [SMILES: CCOC(=O)c1cn(C)nc1Nc1ncc(C(=O)Nc2c(C)ccc(OC)c2C)s1]

A mixture of 2-bromo-N-(3-methoxy-2,6-dimethyl-phenyl)thiazole-5-carboxamide (100 mg, 0.293 mmol, 1.0 eq), ethyl 3-amino-1-methyl-1H-pyrazole-4-carboxylate (59 mg, 0.352 mmol, 1.2 eq), tris(dibenzylideneacetone) dipalladium (0)(40 mg, 0.0440 mmol, 0.15 eq), rac-BINAP (37 mg, 0.0586 mmol, 0.2 eq) and sodium tert-butoxide (42 mg, 0.440 mmol, 1.5 eq) in PhMee (2.9 mL) was heated to 130° C. for 15 min in the MW. The mixture was cooled, sat. aq. Na₂CO₃ (10 mL) added followed by EtOAc (10 mL). The phases were separated, the aqueous phase was washed with EtOAc (10 mL), the extracts combined, washed with brine (20 mL), dried (MgSO₄), filtered and concentrated in vacuo. The resulting residue was chromatographed (SiO₂) eluting with 0-100% EtOAc: PE then subsequent reverse phase chromatography (C18) eluting with 10-95% MeCN: H₂O to afford the title compound (21 mg, 0.0496 mmol, 17%) as a white powder. MS (ES+) m/z 430.2 (M+H). ¹H NMR (300 MHz, DMSO-d₆) δ 9.72 (s, 1H), 9.56 (s, 1H), 8.32 (s, 1H), 8.23-8.17 (m, 1H), 7.08 (d, J=8.4 Hz, 1H), 6.86 (d, J=8.4 Hz, 1H), 4.29 (q, J=7.1 Hz, 2h), 3.87 (s, 3H), 3.79 (s, 3H), 2.13 (s, 3H), 2.03 (s, 3H), 1.31 (t, J=7.1 Hz, 3H).

General Method J: Pd Coupling of 2-Bromo-N-(3-Methoxy-2,6-Dimethyl-Phenyl)Thiazole-5-Carboxamide with Substituted Amino Pyrazoles and In-Situ Ester Hydrolysis:

To a mixture of 2-bromo-N-(3-methoxy-2,6-dimethyl-phenyl)thiazole-5-carboxamide (1.0 eq)amino pyrazole (2.5-3.0 eq) in 1,4-dioxane and water (9:1, 0.09-0.14 M), evacuated and purged with nitrogen, was added Cs₂CO₃ (3.0 eq), Pd₂dba₃ (0.15 eq) and XantPhos (0.25 eq) and heated to 100° C. for 18 h. The mixture was filtered through a pad of dicalite, washed with EtOAc: MeOH and the filtrate was concentrated in vacuo. The residue was treated with sat. aq. Na₂CO₃ to pH 10 and the aqueous phase washed with DCM (x 2) and with EtOAc (x 2). The aqueous phase was acidified with aq. HCl to pH 2, extracted with EtOAc (x 4) and the combined organic layers dried (MgSO₄), filtered and concentrated in vacuo. No further purification was required. Optionally, the crude may be purified by chromatography or other methods if required.

### 2-[3-[[5-[(3-Methoxy-2,6-dimethyl-phenyl) carbamoyl]thiazol-2-yl]amino]pyrazol-1-yl]acetic acid

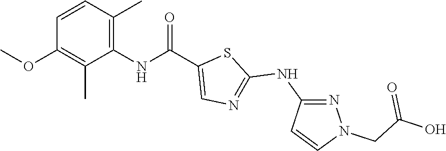 [SMILES: COc1ccc(C)c(NC(=O)c2cnc(Nc3ccn(CC(=O)O)n3)s2)c1C]

Prepared as described in Method J from 2-bromo-N-(3-methoxy-2,6-dimethyl-phenyl)thiazole-5-carboxamide (1.2 g, 3.52 mmol, 1.0 eq), ethyl 2-(3-aminopyrazol-1-yl)acetate
(1.79 g, 10.6 mmol, 3.0 eq), Cs₂CO₃ (3.50 g, 10.6 mmol, 3.0 eq), Pd₂dba₃ (483 mg, 0.528 mmol, 0.15 eq) and XantPhos (509 mg, 0.879 mmol, 0.25 eq) in 1,4-dioxane (36 mL) and water (4 mL) to afford the title compound (1.1 g, 2.74 mmol, 78%) as a light brown solid. MS (ES-) m/z 400.0 (M−H). ¹H NMR (300 MHz, DMSO-d₆) δ 13.16-12.76 (br s, 1H), 11.21-11.01 (br s, 1H), 9.59 (s, 1H), 8.11 (s, 1H), 7.67 (d, J=2.4 Hz, 1H), 7.07 (d, J=8.4 Hz, 1H), 6.85 (d, J=8.4 Hz, 1H), 6.01 (d, J=2.4 Hz, 1H), 4.89 (s, 2H), 3.78 (s, 3H), 2.12 (s, 3H), 2.02 (s, 3H).

The following example compounds were prepared similarly using Method J using the appropriately substituted amine.

| Structure & Name | Amine | Analytical Data |
|---|---|---|
| 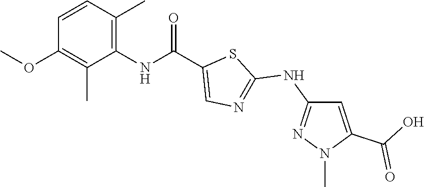 [SMILES: COc1ccc(C)c(NC(=O)c2cnc(Nc3cc(C(=O)O)n(C)n3)s2)c1C] | 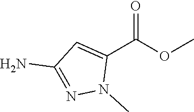 | MS (ES+) m/z 401.9 (M + H). ¹H NMR (300 MHz, DMSO-d₆) δ 13.70-13.30 (br s, 1H), 11.30-11.10 (br s, 1H), 9.64 (s, 1H), 8.13 (s, 1H), 7.07 (d, J = 8.4 Hz, 1H), 6.85 (d, J = 8.4 Hz, 1H), 6.58 (s, 1H), 4.04 (s, 3H), 3.78 (s, 3H), 2.12 (s, 3H), 2.02 (s, 3H). |
| 5-[[5-[(3-methoxy-2,6-dimethyl- | | |
| phenyl)carbamoyl]thiazol-2-yl]amino]- | | |
| 2-methyl-pyrazole-3-carboxylic acid | | |
| 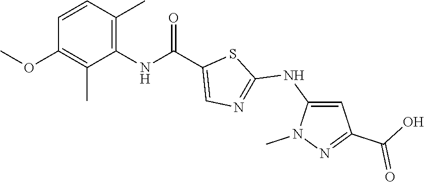 [SMILES: COc1ccc(C)c(NC(=O)c2cnc(Nc3cc(C(=O)O)nn3C)s2)c1C] | 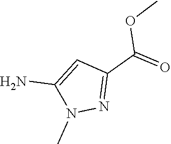 | MS (ES+) m/z 401.9 (M + H). ¹H NMR (300 MHz, DMSO-d₆) δ 12.57-11.97 (br s, 2H), 9.69 (s, 1H), 8.09 (s, 1H), 7.08 (d, J = 8.4 Hz, 1H), 6.86 (d, J = 8.4 Hz, 1H), 6.68 (s, 1H), 3.80 (s, 3H), 3.78 (s, 3H), 2.11 (s, 3H), 2.01 (s, 3H). |
| 5-[[5-[(3-methoxy-2,6-dimethyl- | | |
| phenyl)carbamoyl]thiazol-2-yl]amino]- | | |
| 1-methyl-pyrazole-3-carboxylic acid | | |
| 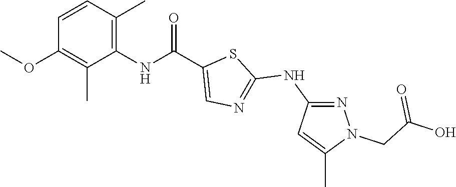 [SMILES: COc1ccc(C)c(NC(=O)c2cnc(Nc3cc(C)n(CC(=O)O)n3)s2)c1C] | 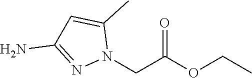 | MS (ES+) m/z 416.3 (M + H). ¹H NMR (300 MHz, DMSO-d₆) δ 13.15-12.95 (brs, 1H), 11.05-10.95 (brs, 1H), 9.57 (s, 1H), 8.10 (s, 1H), 7.07 (d, J = 8.4 Hz, 1H), 6.84 (d, J = 8.4 Hz, 1H), 5.84 (d, J = 0.9 Hz, 1H), 4.82 (s, 2H), 3.78 (s, 3H), 2.20 (s, 3H), 2.12 (s, 3H), 2.02 (s, 3H). |
| 2-[3-[[5-[(3-methoxy-2,6-dimethyl- | | |
| phenyl)carbamoyl]thiazol-2-yl]amino]- | | |
| 5-methyl-pyrazol-1-yl]acetic acid | | |
| 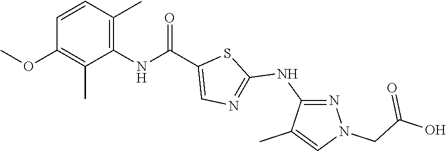 [SMILES: COc1ccc(C)c(NC(=O)c2cnc(Nc3nn(CC(=O)O)cc3C)s2)c1C] | 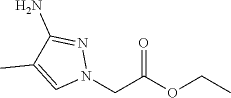 | MS (ES+) m/z 416.0 (M + H). ¹H NMR (300 MHz, DMSO-d₆) δ 13.16-12.76 (brs, 1H), 10.97-10.57 (brs, 1H), 9.56 (s, 1H), 8.12 (s, 1H), 7.47 (s, 1H), 7.07 (d, J = 8.4 Hz, 1H), 6.84 (d, J = 8.4 Hz, |
| | | 1H), 4.83 (s, 2H), |
| 2-[3-[[5-[(3-methoxy-2,6-dimethyl- | | 3.78 (s, 3H), 2.12 |
| phenyl)carbamoyl]thiazol-2-yl]amino]- | | (s, 3H), 2.02 (s, 6H). |
| 4-methyl-pyrazol-1-yl]acetic acid | | |

### 2-(3-((5-((3-Methoxy-2,6-dimethylphenyl)carbamoyl)thiazol-2-yl)amino)-1H-pyrazol-1-yl)propanoic acid

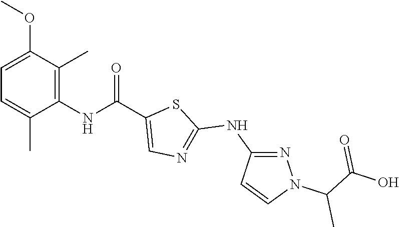 [SMILES: COc1ccc(C)c(NC(=O)c2cnc(Nc3ccn(C(C)C(=O)O)n3)s2)c1C]

To a solution of tert-butyl 2-[3-[5-[(3-methoxy-2,6-dimethyl-phenyl) carbamoyl]thiazol-2-yl]amino]pyrazol-1-yl]propanoate (2.85 g, 3.93 mmol) in DCM (6 mL) was added trifluoroacetic acid

(3.0 mL, 39.3 mmol) and the reaction was stirred for 2.5 h, then concentrated under reduced pressure and taken up in saturated aqueous NaHCO₃ (100 mL). The mixture was washed with MTBE (3×30 mL), acidified with conc. HCl then extracted with EtOAc (2×50 mL). The combined organic layers were dried over sodium sulfate, filtered then concentrated under reduced pressure to afford the title compound (1.95 g, 2.82 mmol, 71.7% yield) as a clear brown oil. MS (ES+) m/z=416.3 [M+H]+. ¹H NMR (400 MHz, DMSO-D6) δ 12.12 (s, 1H), 9.57 (s, 1H), 8.11 (s, 1H), 7.06 (d, J=8.4 Hz, 1H), 6.84 (d, J=8.4 Hz, 1H), 6.01 (d, J=2.4 Hz, 1H), 5.08 (dq, J=23.7, 7.2 Hz, 1H), 3.77 (s, 3H), 2.11 (s, 3H), 2.01 (s, 3H), 1.91 (s, 3H).

### 2-(3-((5-((3-Methoxy-2,6-dimethylphenyl)carbamoyl)thiazol-2-yl)amino)-1H-pyrazol-1-yl) butanoic acid

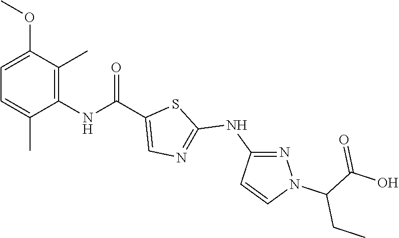 [SMILES: CCC(C(=O)O)n1ccc(Nc2ncc(C(=O)Nc3c(C)ccc(OC)c3C)s2)n1]

To a solution of tert-butyl 2-[3-[5-[(3-methoxy-2,6-dimethyl-phenyl) carbamoyl]thiazol-2-yl]amino]pyrazol-1-yl]butanoate (3.67 g, 4.31 mmol) in DCM (6.6 mL) was added trifluoroacetic acid (3.3 mL, 43.1 mmol) and the reaction was stirred for 2.5 h then concentrated under reduced pressure and taken up in saturated aqueous NaHCO₃ (100 mL). The mixture was washed with MTBE (3×30 mL), acidified with conc. HCl then extracted with EtOAc (2×50 mL). The combined organic layers were dried over sodium sulfate, filtered then concentrated under reduced pressure to afford the title compound (970 mg, 2.19 mmol, 50.85% yield) as a beige solid. MS (ES+) m/z=430.1 [M+H]⁺, ¹H NMR (400 MHz, DMSO-D6) δ 11.20 (s, 1H), 9.61 (s, 1H), 8.14 (s, 1H), 7.74 (d, J=2.4 Hz, 1H), 7.06 (d, J=8.4 Hz, 1H), 6.84 (d, J=8.4 Hz, 1H), 6.02 (d, J=2.4 Hz, 1H), 4.83 (dd, J=9.2, 6.0 Hz, 1H), 3.77 (s, 3H), 2.18-2.05 (m, 5H), 2.01 (s, 3H), 0.81 (t, J=7.3 Hz, 3H).

### 2-(3-((5-((3-Methoxy-2,6-dimethylphenyl)carbamoyl)thiazol-2-yl)amino)-4-methyl-1H-pyrazol-1-yl)propanoic acid

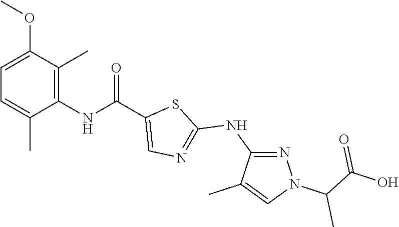 [SMILES: COc1ccc(C)c(NC(=O)c2cnc(Nc3nn(C(C)C(=O)O)cc3C)s2)c1C]

To a solution of tert-butyl 2-[3-[5-[(3-methoxy-2,6-dimethyl-phenyl) carbamoyl]thiazol-2-yl]amino]-4-methyl-pyrazol-1-yl]propanoate (1.79 g, 2.92 mmol) in DCM (20 mL) was added trifluoroacetic acid (10 mL, 131 mmol) and the reaction was stirred for 2.5 h, concentrated under reduced pressure then taken up in saturated aqueous NaHCO₃ (30 mL). The mixture was washed with DCM, acidified with conc. HCl then filtered under reduced pressure. The residue was dissolved in 4 M HCl in 1,4-dioxane (10 mL) and stirred for 30 min, the solution was then concentrated under reduced pressure to afford the title compound (1.01 g, 2.30 mmol, 78.96% yield) as an off-white solid. MS (ES-) m/z=428.2 [M-H]−. ¹H NMR (400 MHz, DMSO-D6) δ 10.85 (s, 1H), 9.57 (s, 1H), 8.13 (s, 1H), 7.55 (d, J=1.0 Hz, 1H), 7.06 (d, J=8.4 Hz, 1H), 6.84 (d, J=8.4 Hz, 1H), 4.98 (q, J=7.2 Hz, 1H), 3.77 (s, 3H), 2.11 (s, 3H), 2.01 (d, J=1.4 Hz, 6H), 1.64 (d, J=7.2 Hz, 3H).

### 2-(3-((5-((3-Methoxy-2,6-dimethylphenyl)carbamoyl)thiazol-2-yl)amino)-5-methyl-1H-pyrazol-1-yl)propanoic acid

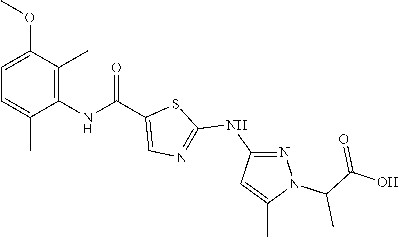 [SMILES: COc1ccc(C)c(NC(=O)c2cnc(Nc3cc(C)n(C(C)C(=O)O)n3)s2)c1C]

To a solution of tert-butyl 2-[3-[5-[(3-methoxy-2,6-dimethyl-phenyl) carbamoyl]thiazol-2-yl]amino]-5-methyl-pyrazol-1-yl]propanoate (2.73 g, 2.59 mmol) in DCM (4 mL) was added trifluoroacetic acid (2.0 mL, 25.9 mmol) and the reaction was stirred for 2.5 h, then concentrated under reduced pressure and taken up in saturated aqueous NaHCO₃ (100 mL). The mixture was washed with MTBE (3×30 mL), acidified with conc. HCl then extracted with EtOAc (2×50 mL). The combined organic layers were dried over sodium sulfate, filtered then concentrated under reduced pressure to afford the title compound (1.49 g, 2.60 mmol, 100.61% yield) as an off-white solid.MS (ES+) m/z=430.4 [M+H]⁺, ¹H NMR (400 MHz, DMSO-D6) δ 12.03 (s, 1H), 9.55 (s, 1H), 8.10 (s, 1H), 7.06 (d, J=8.4 Hz, 1H), 6.84 (d, J=8.4 Hz, 1H), 5.81 (s, 1H), 5.16-4.97 (m, 1H), 3.77 (s, 3H), 2.22 (s, 3H), 2.11 (s, 3H), 1.99 (t, J=0.6 Hz, 3H), 1.65 (d, J=7.0 Hz, 3H).

### 3-Methoxy-2-(3-((5-((3-methoxy-2,6-dimethylphenyl)carbamoyl)thiazol-2-yl)amino)-4-methyl-1H-pyrazol-1-yl)propanoic acid

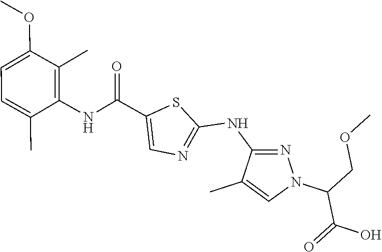

Methyl 3-methoxy-2-[3-[5-[(3-methoxy-2,6-dimethyl-phenyl) carbamoyl]thiazol-2-yl]amino]-4-methyl-pyrazol-1-yl]propanoate (1.00 eq, 150 mg, 0.253 mmol) was dissolved in a mixture of 1,4-Dioxane (10 mL) and H₂O (1.0 mL), and caesium carbonate (1.45 eq, 120 mg, 0.368 mmol) was added. The resulting mixture was heated to 100° C. and left to stir for 18 h, then was concentrated under reduced pressure and dried in a vacuum oven at 40° C. to afford caesium 3-methoxy-2-[3-[[5-[(3-methoxy-2,6-dimethyl-phenyl) carbamoyl]thiazol-2-yl]amino]-4-methyl-pyrazol-1-yl]propanoate (230 mg, 0.253 mmol, 99.75% yield) as a brown solid without further purification. MS (ES+) m/z=460.3, [M+H]+. ¹H NMR (400 MHz, DMSO-d₆) δ 7.89 (s, 1H), 7.28 (s, 1H), 7.01 (d, J=8.4 Hz, 1H), 6.78 (d, J=8.3 Hz, 1H), 4.44 (dd, J=8.6, 4.1 Hz, 1H), 3.89-3.81 (m, 2H), 3.76 (s, 3H), 2.11 (s, 3H), 2.03 (s, 3H), 1.94 (s, 3H), 1.51 (s, 3H).

### Amide Formation with 5-[[5-[(3-methoxy-2,6-dimethyl-phenyl) carbamoyl]thiazol-2-yl]amino]-2-methyl-pyrazole-3-carboxylic acid

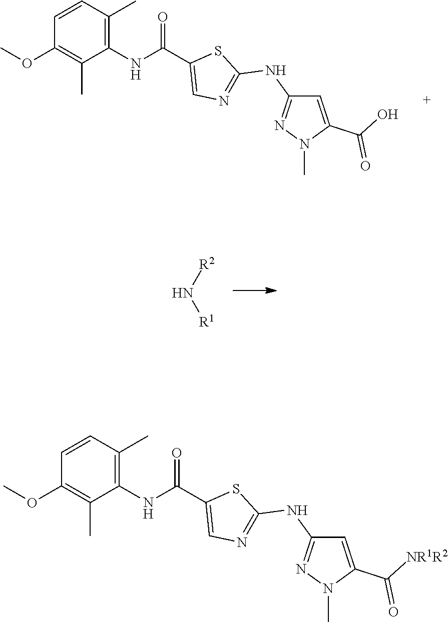

### N-(3-Methoxy-2,6-dimethyl-phenyl)-2-[[1-methyl-5-(methylcarbamoyl)pyrazol-3-yl]amino]thiazole-5-carboxamide

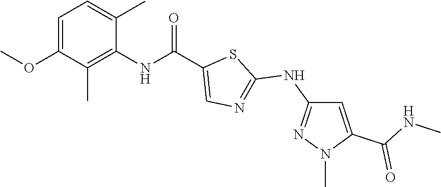

A mixture of 5-[5-[(3-methoxy-2,6-dimethyl-phenyl) carbamoyl]thiazol-2-yl]amino]-2-methyl-pyrazole-3-carboxylic acid (40 mg, 0.0996 mmol, 1.0 eq), methylamine hydrochloride (13 mg, 0.199 mmol, 2.0 eq), HATU (57 mg, 0.149 mmol, 1.5 eq) and DIPEA (0.069 mL, 0.399 mmol, 4.0 eq) in anhydrous DMF (1 mL) under nitrogen was stirred at RT for 18 h. The mixture was partitioned between EtOAc and water. The aqueous phase was extracted with EtOAc (x 2), and the combined organic layers were washed with brine, dried (MgSO₄), filtered and concentrated under reduced pressure. The crude material was chromatographed (SiO₂) eluting with 0-20% MeOH: DCM to afford the title compound (30 mg, 0.0724 mmol, 73%) as a light beige solid. MS (ES+) m/z 414.9 (M+H).¹H NMR (300 MHz, DMSO-d₆) δ 11.22 (s, 1H), 9.62 (s, 1H), 8.56 (q, J=4.4 Hz, 1H), 8.12 (s, 1H), 7.07 (d, J=8.4 Hz, 1H), 6.85 (d, J=8.4 Hz, 1H), 6.60 (s, 1H), 4.01 (s, 3H), 3.78 (s, 3H), 2.74 (d, J=4.5 Hz, 3H), 2.12 (s, 3H), 2.02 (s, 3H).

### 2-[[5-(Dimethylcarbamoyl)-1-methyl-pyrazol-3-yl]amino]-N-(3-methoxy-2,6-dimethyl-phenyl)thiazole-5-carboxamide

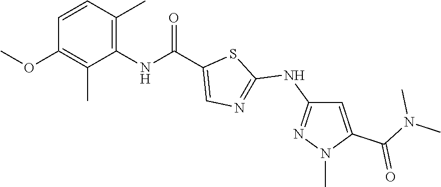

Prepared similarly from 5-[[5-[(3-methoxy-2,6-dimethyl-phenyl) carbamoyl]thiazol-2-yl]amino]-2-methyl-pyrazole-3-carboxylic acid (40 mg, 0.0996 mmol, 1.0 eq) with dimethylamine hydrochloride (16 mg, 0.199 mmol, 2.0 eq) to afford the title compound (25 mg, 0.0583 mmol, 59%) as a light beige solid after normal phase chromatography (SiO₂) eluting with 0-20% MeOH: DCM. MS (ES+) m/z 428.9 (M+H). ¹H NMR (300 MHz, DMSO-d₆) δ 11.15 (s, 1H), 9.62 (s, 1H), 8.12 (s, 1H), 7.07 (d, J=8.4 Hz, 1H), 6.85 (d, J=8.4 Hz, 1H), 6.19 (s, 1H), 3.78 (s, 6H), 3.05 (s, 3H), 3.00 (s, 3H), 2.12 (s, 3H), 2.02 (s, 3H).

De-Methylation with BBr₃

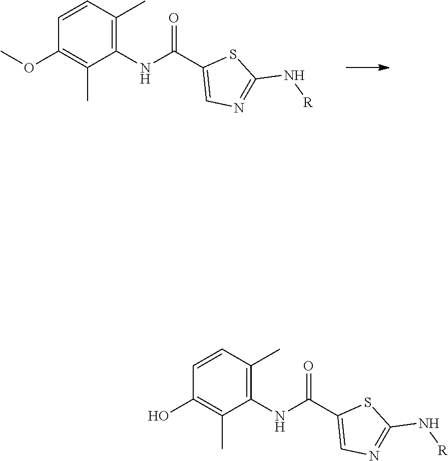

General Method K: Methoxy Deprotection with BBr₃

To a suspension of methoxy analogue (1 eq) in DCM (0.01-0.08 M) was added BBr₃ (1M in DCM, 3.0-4.1 eq) at 0° C. The reaction was monitored by consumption of starting material via UPLC. Additional BBr₃ added to push the reaction to completion. Water added to the mixture and stirred vigorously for 10 min. aq. or solid NaHCO₃added to pH 7-8 and the phases separated. The aqueous phase was extracted with EtOAc (x 3-4) and the combined organic layers were dried (MgSO₄), filtered and concentrated under reduced pressure. The crude material was purified by normal phase chromatography (SiO₂) using a gradient of MeOH: DCM or by preparative HPLC-MS using a gradient of high or low pH aq. MeCN.

Where the product was a carboxylic acid, either the product was extracted directly or the aqueous phase after washing with EtOAc was acidified to pH 1-2 with aq. HCl (2M) then re-extracted with EtOAc (x 3) and the combined organic layers were dried (MgSO₄), filtered and concentrated under reduced pressure. No further purification was required.

### 2-[[1-[2-(Dimethylamino)-2-oxo-ethyl]pyrazol-3-yl]amino]-N-(3-hydroxy-2,6-dimethyl-phenyl)thiazole-5-carboxamide (BAA-117)

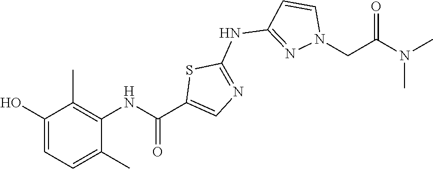 [SMILES: Cc1ccc(O)c(C)c1NC(=O)c1cnc(Nc2ccn(CC(=O)N(C)C)n2)s1]

Prepared as described in Method K from 2-[1-[2-(dimethylamino)-2-oxo-ethyl]pyrazol-3-yl]amino]-N-(3-methoxy-2,6-dimethyl-phenyl)thiazole-5-carboxamide (35 mg, 0.0817 mmol, 1.0 eq) and BBr₃ (1M in DCM, 0.25 mL, 0.245 mmol, 3.0 eq) at 0° C. in DCM (5 mL) to afford the title compound (20 mg, 0.0483 mmol, 59%) as a white solid after normal phase chromatography (SiO₂) eluting with 0-20% MeOH: DCM. MS (ES+) m/z 414.9 (M+H). ¹H NMR (300 MHz, DMSO-d₆) δ 11.05 (s, 1H), 9.51 (s, 1H), 9.19 (s, 1H), 8.10 (s, 1H), 7.58 (d, J=2.3 Hz, 1H), 6.89 (d, J=8.2 Hz, 1H), 6.68 (d, J=8.2 Hz, 1H), 5.98 (d, J=2.4 Hz, 1H), 5.04 (s, 2H), 3.05 (s, 3H), 2.85 (s, 3H), 2.07 (s, 3H), 1.99 (s, 3H). LCMS, High pH: RT=0.76, MS (ES-) m/z 413.0 (M−H).

The following example compounds were prepared similarly using Method K:

| # | Structure & Name | Analytical Data |
|---|---|---|
| BAA-059 | 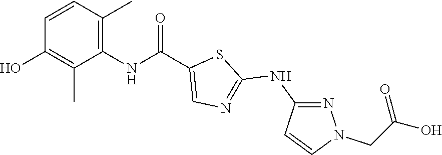 [SMILES: Cc1ccc(O)c(C)c1NC(=O)c1cnc(Nc2ccn(CC(=O)O)n2)s1] | MS (ES−) m/z 386.0 (M − H). ¹H NMR (300 MHz, DMSO-d₆) δ 13.19-12.79 (br s, 1H), 11.20- 11.00 (br s, 1H), 9.52 (s, 1H), 9.24- 9.14 (br s, 1H), 8.10 (s, 1H), 7.67 (d, J = 2.4 Hz, 1H), 6.89 (d, J = 8.2 Hz, 1H), 6.68 (d, J = 8.2 Hz, 1H), 6.01 (d, J = 2.4 Hz, 1H), 4.89 (s, 2H), 2.07 (s, 3H), 1.99 (s, 3H). |
| | 2-[3-[[5-[(3-hydroxy-2,6-dimethyl- | |
| | phenyl)carbamoyl]thiazol-2- | |
| | yl]amino]pyrazol-1-yl]acetic acid | |
| BAA-118 | 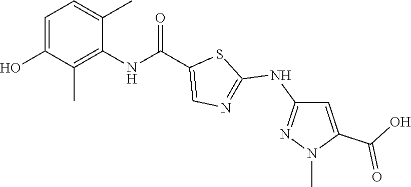 [SMILES: Cc1ccc(O)c(C)c1NC(=O)c1cnc(Nc2cc(C(=O)O)n(C)n2)s1] | MS (ES+) m/z 387.9 (M + H). ¹H NMR (300 MHz, DMSO-d₆) δ 13.72-13.32 (br s, 1H), 11.27- 11.07 (br s, 1H), 9.56 (s, 1H), 9.20 (s, 1H), 8.11 (s, 1H), 6.88 (s, 1H), 6.69 (d, J = 8.2 Hz, 1H), 6.58 (s, 1H), 4.04 (s, 3H), 2.07 (s, 3H), 1.99 (s, 3H). |
| | 5-[[5-[(3-hydroxy-2,6-dimethyl- | |
| | phenyl)carbamoyl]thiazol-2-yl]amino]- | |
| | 2-methyl-pyrazole-3-carboxylic acid | |
| BAA-119 | 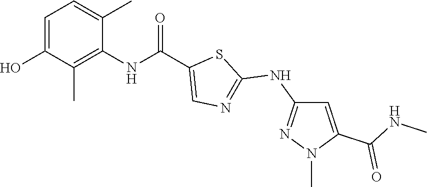 [SMILES: CNC(=O)c1cc(Nc2ncc(C(=O)Nc3c(C)ccc(O)c3C)s2)nn1C] | MS (ES+) m/z 400.9 (M + H). ¹H NMR (300 MHz, DMSO-d₆) δ 11.21 (s, 1H), 9.54 (s, 1H), 9.21 (s, 1H), 8.56 (q, J = 4.1 Hz, 1H), 8.11 (s, 1H), 6.89 (d, J = 8.2 Hz, 1H), 6.69 (d, J = 8.2 Hz, 1H), 6.61 (s, 1H), 4.01 (s, 3H), 2.74 (d, J = 4.5 Hz, 3H), 2.07 (s, 3H), 1.99 (s, 3H). |
| | N-(3-hydroxy-2,6-dimethyl-phenyl)-2- | |
| | [[1-methyl-5- | |
| | (methylcarbamoyl)pyrazol-3- | |
| | yl]amino]thiazole-5-carboxamide | |
| BAA-120 | 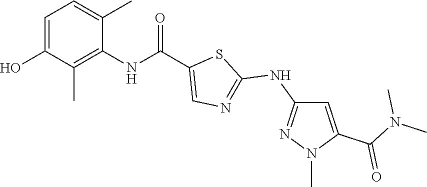 [SMILES: Cc1ccc(O)c(C)c1NC(=O)c1cnc(Nc2cc(C(=O)N(C)C)n(C)n2)s1] | MS (ES+) m/z 414.9 (M + H). ¹H NMR (300 MHz, DMSO-d₆) δ 11.13 (s, 1H), 9.54 (s, 1H), 9.20 (s, 1H), 8.11 (s, 1H), 6.89 (d, J = 8.2 Hz, 1H), 6.69 (d, J = 8.2 Hz, 1H), 6.19 (s, 1H), 3.78 (s, 3H), 3.05 (s, 3H), 3.00 (s, 3H), 2.07 (s, 3H), 1.99 (s, 3H). |
| | 2-[[5-(dimethylcarbamoyl)-1-methyl- | |
| | pyrazol-3-yl]amino]-N-(3-hydroxy-2,6- | |
| | dimethyl-phenyl)thiazole-5- | |
| | carboxamide | |
| BAA-121 | 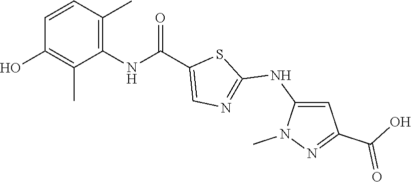 [SMILES: Cc1ccc(O)c(C)c1NC(=O)c1cnc(Nc2cc(C(=O)O)nn2C)s1] | MS (ES+) m/z 387.9 (M + H). ¹H NMR (300 MHz, DMSO-d₆) δ 12.71-12.11 (br s, 2H), 9.62 (s, 1H), 9.23 (s, 1H), 8.08 (s, 1H), 6.89 (d, J = 8.2 Hz, 1H), 6.69 (d, J = 8.2 Hz, 2H), 3.80 (s, 3H), 2.06 (s, 3H), 1.98 (s, 3H). |
| | 5-[[5-[(3-hydroxy-2,6-dimethyl- | |
| | phenyl)carbamoyl]thiazol-2-yl]amino]- | |
| | 1-methyl-pyrazole-3-carboxylic acid | |
| BAA-122 | 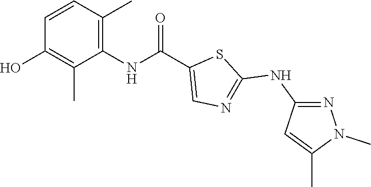 [SMILES: Cc1ccc(O)c(C)c1NC(=O)c1cnc(Nc2cc(C)n(C)n2)s1] | MS (ES+) m/z 358.2 (M + H). ¹H NMR (300 MHz, DMSO-d₆) δ 10.92 (s, 1H), 9.48 (s, 1H), 9.18 (s, 1H), 8.08 (s, 1H), 6.88 (d, J = 8.2 Hz, 1H), 6.68 (d, J = 8.2 Hz, 1H), 5.78 (d, J = 0.8 Hz, 1H), 3.67 (s, 3H), 2.23 (s, 3H), 2.07 (s, 3H), 1.99 (s, 3H). |
| | 2-[(1,5-dimethylpyrazol-3-yl)amino]-N- | |
| | (3-hydroxy-2,6-dimethyl- | |
| | phenyl)thiazole-5-carboxamide | |
| BAA-123 | 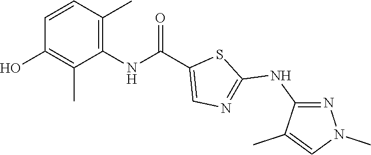 [SMILES: Cc1cn(C)nc1Nc1ncc(C(=O)Nc2c(C)ccc(O)c2C)s1] | MS (ES+) m/z 357.9 (M + H). ¹H NMR (300 MHz, DMSO-d₆) δ 10.67 (s, 1H), 9.47 (s, 1H), 9.18 (s, 1H), 8.09 (s, 1H), 7.43 (d, J = 0.9 Hz, 1H), 6.88 (d, J = 8.2 Hz, 1H), 6.68 (d, J = 8.2 Hz, 1H), 3.74 (s, 3H), 2.07 (s, 3H), 1.99 (s, 6H). |
| | 2-[(1,4-dimethylpyrazol-3-yl)amino]-N- | |
| | (3-hydroxy-2,6-dimethyl- | |
| | phenyl)thiazole-5-carboxamide | |
| BAA-124 | 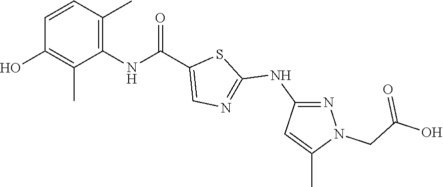 [SMILES: Cc1ccc(O)c(C)c1NC(=O)c1cnc(Nc2cc(C)n(CC(=O)O)n2)s1] | MS (ES+) m/z 402.3 (M + H). ¹H NMR (300 MHz, DMSO-d₆) δ 13.27-12.87 (brs, 1H), 11.10- 10.90 (brs, 1H), 9.50 (s, 1H), 9.28- 9.08 (brs, 1H), 8.09 (s, 1H), 6.88 (d, J = 8.2 Hz, 1H), 6.68 (d, J = 8.2 Hz, 1H), 5.84 (s, 1H), 4.82 (s, 2H), 2.20 (s, 3H), 2.07 (s, 3H), 1.98 (s, 3H). |
| | 2-[3-[[5-[(3-hydroxy-2,6-dimethyl- | |
| | phenyl)carbamoyl]thiazol-2-yl]amino]- | |
| | 5-methyl-pyrazol-1-yl]acetic acid | |
| BAA-125 | 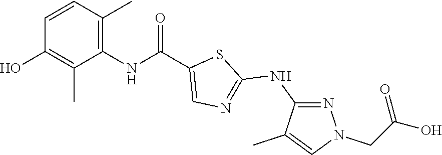 [SMILES: Cc1cn(CC(=O)O)nc1Nc1ncc(C(=O)Nc2c(C)ccc(O)c2C)s1] | MS (ES+) m/z 401.9 (M + H). ¹H NMR (300 MHz, DMSO-d₆) δ 13.12-12.72 (brs, 1H), 10.98- 10.58 (brs, 1H), 9.49 (s, 1H), 9.28- 9.08 (brs, 1H), 8.11 (s, 1H), 7.48 (d, J = 1.0 Hz, 1H), 6.88 (d, J = 8.2 Hz, 1H), 6.68 (d, J = 8.2 Hz, 1H), 4.83 (s, 2H), 2.07 (s, 3H), 2.01 (s, 3H), 1.99 (s, 3H). |
| | 2-[3-[[5-[(3-hydroxy-2,6-dimethyl- | |
| | phenyl)carbamoyl]thiazol-2-yl]amino]- | |
| | 4-methyl-pyrazol-1-yl]acetic acid | |
| BAA-126 | 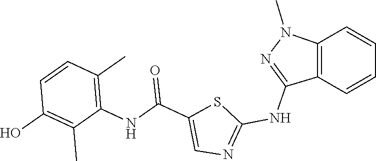 [SMILES: Cc1ccc(O)c(C)c1NC(=O)c1cnc(Nc2nn(C)c3ccccc23)s1] | MS (ES+) m/z 394.3 (M + H). ¹H NMR (300 MHz, DMSO-d₆) δ 11.86 (s, 1H), 9.56 (s, 1H), 9.17 (s, 1H), 8.21 (s, 1H), 8.11 (d, J = 8.2 Hz, 1H), 7.56 (d, J = 8.6 Hz, 1H), 7.50-7.39 (m, 1H), 7.17-7.06 (m, 1H), 6.90 (d, J = 8.2 Hz, 1H), 6.70 (d, J = 8.2 Hz, 1H), 3.99 (s, 3H), 2.10 (s, 3H), 2.02 (s, 3H). |
| | N-(3-hydroxy-2,6-dimethyl-phenyl)-2- | |
| | [(1-methylindazol-3-yl)amino]thiazole- | |
| | 5-carboxamide | |
| BAA-127 | 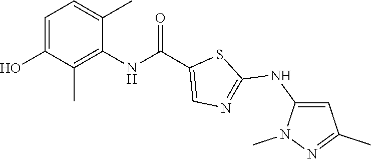 [SMILES: Cc1cc(Nc2ncc(C(=O)Nc3c(C)ccc(O)c3C)s2)n(C)n1] | MS (ES+) m/z 358.3 (M + H). ¹H NMR (300 MHz, DMSO-d₆) δ 10.66-10.26 (brs, 1H), 9.55 (s, 1H), 9.18 (s, 1H), 8.04 (s, 1H), 6.88 (d, J = 8.2 Hz, 1H), 6.69 (d, J = 8.2 Hz, 1H), 6.21-6.01 (brs, 1H), 3.63 (s, 3H), 2.12 (s, 3H), 2.06 (s, 3H), 1.98 (s, 3H). |
| | 2-[(2,5-dimethylpyrazol-3-yl)amino]-N- | |
| | (3-hydroxy-2,6-dimethyl- | |
| | phenyl)thiazole-5-carboxamide | |
| BAA-128 | 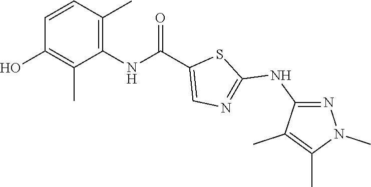 [SMILES: Cc1ccc(O)c(C)c1NC(=O)c1cnc(Nc2nn(C)c(C)c2C)s1] | MS (ES+) m/z 372.2 (M + H). ¹H NMR (300 MHz, DMSO-d₆) δ 10.56 (s, 1H), 9.43 (s, 1H), 9.15 (s, 1H), 8.08 (s, 1H), 6.88 (d, J = 8.2 Hz, 1H), 6.68 (d, J = 8.1 Hz, 1H), 3.66 (s, 3H), 2.15 (s, 3H), 2.07 (s, 3H), 1.99 (s, 3H), 1.92 (s, 3H). |
| | N-(3-hydroxy-2,6-dimethyl-phenyl)-2- | |
| | [(1,4,5-trimethylpyrazol-3- | |
| | yl)amino]thiazole-5-carboxamide | |
| BAA-129 | 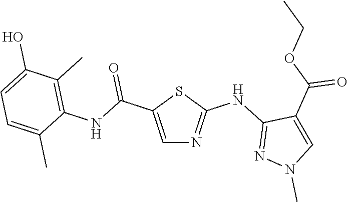 [SMILES: CCOC(=O)c1cn(C)nc1Nc1ncc(C(=O)Nc2c(C)ccc(O)c2C)s1] | MS (ES+) m/z 416.1 (M + H). ¹H NMR (300 MHz, DMSO-d₆) δ 9.64 (s, 1H), 9.53 (s, 1H), 9.18 (s, 1H), 8.32 (s, 1H), 8.19 (s, 1H), 6.89 (d, J = 8.2 Hz, 1H), 6.69 (d, J = 8.2 Hz, 1H), 4.29 (q, J = 7.1 Hz, 2H), 3.87 (s, 3H), 2.08 (s, 3H), 2.00 (s, 3H), 1.31 (t, J = 7.1 Hz, 3H). |
| | ethyl 3-[[5-[(3-hydroxy-2,6-dimethyl- | |
| | phenyl)carbamoyl]thiazol-2-yl]amino]- | |
| | 1-methyl-pyrazole-4-carboxylate | |
| BAA-210 | 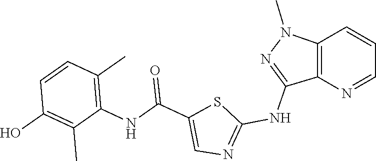 [SMILES: Cc1ccc(O)c(C)c1NC(=O)c1cnc(Nc2nn(C)c3cccnc23)s1] | ¹H NMR (300 MHz, DMSO-d₆) δ 9.58 (s, 1H), 9.17 (br, 1H), 8.50 (m, 1H), 8.20 (s, 1H), 8.09 (m, 1H), 7.47 (dd, J = 8.6, 4.3 Hz, 1H), 6.89 (d, J = 8.2 Hz, 1H), 6.69 (d, J = 8.2 Hz, 1H), 4.02 (s, 3H), 2.09 (s, 3H), 2.01 (s, 3H). [1H hidden under water] MS (ES+) m/z 395.3 (M + H)⁺ |
| | N-(3-hydroxy-2,6-dimethylphenyl)-2- | |
| | ((1-methyl-1H-pyrazolo[4,3-b]pyridin- | |
| | 3-yl)amino)thiazole-5-carboxamide | |
| BAA-454 | 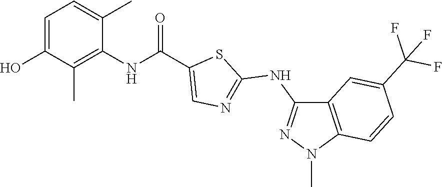 [SMILES: Cc1ccc(O)c(C)c1NC(=O)c1cnc(Nc2nn(C)c3ccc(C(F)(F)F)cc23)s1] | ¹H NMR (300 MHz, DMSO) δ 12.07 (s, 1H), 9.62 (s, 1H), 9.20 (s, 1H), 8.65 (s, 1H), 8.24 (s, 1H), 7.79 (d, J = 9.0 Hz, 1H), 7.71 (dd, J = 9.1, 1.7 Hz, 1H), 6.89 (d, J = 8.2 Hz, 1H), 6.69 (d, J = 8.2 Hz, 1H), 4.05 (s, 3H), 2.08 (s, 3H), 2.00 (s, 3H); ¹⁹F NMR (282 MHz, DMSO) δ 58.69. MS (ES+) m/z 462.3 (M + H)⁺. |
| | N-(3-hydroxy-2,6-dimethylphenyl)-2- | |
| | ((1-methyl-5-(trifluoromethyl)-1H- | |
| | indazol-3-yl)amino)thiazole-5- | |
| | carboxamide | |
| BAA-245 | 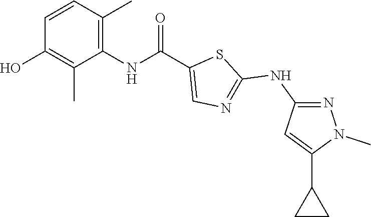 [SMILES: Cc1ccc(O)c(C)c1NC(=O)c1cnc(Nc2cc(C3CC3)n(C)n2)s1] | ¹H NMR (300 MHz, DMSO-d₆) δ 10.42 (s, 1H), 9.54 (s, 1H), 9.17 (s, 1H), 8.03 (s, 1H), 6.89 (d, J = 8.2 Hz, 1H), 6.69 (d, J = 8.2 Hz, 1H), 6.04 (s, 1H), 3.61 (s, 3H), 2.06 (s, 3H), 1.98 (s, 3H), 1.81 (tt, J = 8.4, 5.1 Hz, 1H), 0.87-0.76 (m, 2H), 0.69-0.58 (m, 2H). MS (ES⁺) m/z 384.1 (M + H)⁺. |
| | 2-((5-cyclopropyl-1-methyl-1H- | |
| | pyrazol-3-yl)amino)-N-(3-hydroxy-2,6- | |
| | dimethylphenyl)thiazole-5- | |
| | carboxamide | |
| BAA-246 | 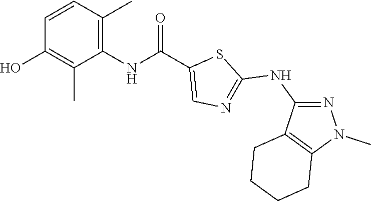 [SMILES: Cc1ccc(O)c(C)c1NC(=O)c1cnc(Nc2nn(C)c3c2CCCC3)s1] | ¹H NMR (300 MHz, DMSO-d₆) δ 10.00 (s, 1H), 9.50 (s, 1H), 9.16 (s, 1H), 7.98 (s, 1H), 6.87 (d, J = 8.3 Hz, 1H), 6.68 (d, J = 8.2 Hz, 1H), 3.58 (s, 3H), 2.34-2.26 (m, 2H), 2.05 (s, 3H), 1.97 (s, 3H), 1.79- 1.59 (m, 4H). MS (ES⁺) m/z 398.1 (M + H)⁺. |
| | N-(3-hydroxy-2,6-dimethylphenyl)-2- | |
| | ((1-methyl-4,5,6,7-tetrahydro-1H- | |
| | indazol-3-yl)amino)thiazole-5- | |
| | carboxamide | |
| BAA-247 | 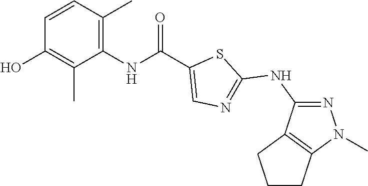 [SMILES: Cc1ccc(O)c(C)c1NC(=O)c1cnc(Nc2nn(C)c3c2CCC3)s1] | ¹H NMR (300 MHz, DMSO-d₆) δ 10.30 (s, 1H), 9.51 (s, 1H), 9.16 (s, 1H), 8.01 (s, 1H), 6.88 (d, J = 8.2 Hz, 1H), 6.68 (d, J = 8.2 Hz, 1H), 3.62 (s, 3H), 2.63-2.53 (m, 4H), 2.32 (q, J = 7.3 Hz, 2H), 2.05 (s, 3H), 1.97 (s, 3H). MS (ES⁺) m/z 384.1 (M + H)⁺. |
| | N-(3-hydroxy-2,6-dimethylphenyl)-2- | |
| | ((1-methyl-1,4,5,6- | |
| | tetrahydrocyclopenta[c]pyrazol-3- | |
| | yl)amino)thiazole-5-carboxamide | |
| BAA-250 | 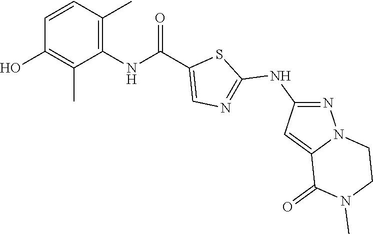 [SMILES: Cc1ccc(O)c(C)c1NC(=O)c1cnc(Nc2cc3n(n2)CCN(C)C3=O)s1] | ¹H NMR (300 MHz, DMSO-d₆) δ 11.14 (s, 1H), 9.55 (s, 1H), 9.18 (s, 1H), 8.12 (s, 1H), 6.89 (d, J = 8.2 Hz, 1H), 6.70 (d, J = 8.2 Hz, 1H), 6.46 (s, 1H), 4.38-4.27 (m, 2H), 3.85-3.75 (m, 2H), 3.02 (s, 3H), 2.08 (s, 3H), 2.00 (s, 3H). MS (ES⁺) m/z 413.0 (M + H)⁺. |
| | N-(3-hydroxy-2,6-dimethylphenyl)-2- | |
| | ((5-methyl-4-oxo-4,5,6,7- | |
| | tetrahydropyrazolo[1,5-a]pyrazin-2- | |
| | yl)amino)thiazole-5-carboxamide | |
| BAA-251 | 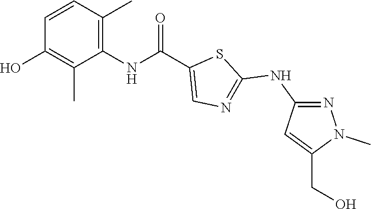 [SMILES: Cc1ccc(O)c(C)c1NC(=O)c1cnc(Nc2cc(CO)n(C)n2)s1] | ¹H NMR (300 MHz, DMSO-d₆) δ 10.95 (s, 1H), 9.48 (s, 1H), 9.16 (s, 1H), 8.09 (s, 1H), 6.89 (d, J = 8.2 Hz, 1H), 6.69 (d, J = 8.2 Hz, 1H), 5.94 (s, 1H), 5.30 (t, J = 5.6 Hz, 1H), 4.48 (d, J = 5.6 Hz, 2H), 3.74 (s, 3H), 2.08 (s, 3H), 2.00 (s, 3H).MS (ES⁺) m/z 374.0 (M + H)⁺. |
| | N-(3-hydroxy-2,6-dimethylphenyl)-2- | |
| | ((5-(hydroxymethyl)-1-methyl-1H- | |
| | pyrazol-3-yl)amino)thiazole-5- | |
| | carboxamide | |
| BAA-483 | 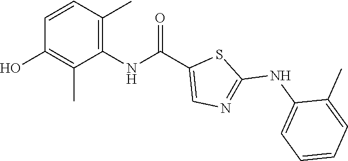 [SMILES: Cc1ccccc1Nc1ncc(C(=O)Nc2c(C)ccc(O)c2C)s1] | ¹H NMR (400 MHz, DMSO-d₆) δ 9.48 (s, 1H), 9.15 (s, 1H), 8.00 (s, 1H), 7.70 (d, J = 7.2 Hz, 1H), 7.26 (d, J = 6.9 Hz, 1H), 7.22 (t, J = 7.6 Hz, 1H), 7.10 (td, J = 7.3, 1.3 Hz, 1H), 6.87 (d, J = 8.1 Hz, 1H), 6.67 (d, J = 8.2 Hz, 1H), 2.26 (s, 3H), 2.05 (s, 3H), 1.97 (s, 3H). MS (ES⁺) m/z 354.1 (M + H)⁺. |
| | N-(3-hydroxy-2,6-dimethylphenyl)-2- | |
| | (o-tolylamino)thiazole-5-carboxamide | |
| BAA-484 | 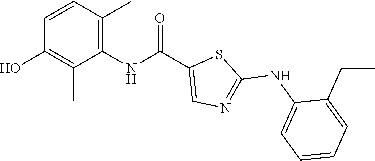 [SMILES: CCc1ccccc1Nc1ncc(C(=O)Nc2c(C)ccc(O)c2C)s1] | ¹H NMR (400 MHz, DMSO-d₆) δ 9.76 (s, 1H), 9.46 (s, 1H), 9.15 (s, 1H), 7.98 (s, 1H), 7.63 (d, J = 8.4 Hz, 1H), 7.29 (dd, J = 7.4, 1.7 Hz, 1H), 7.24 (td, J = 7.7, 1.8 Hz, 1H), 7.17 (td, J = 7.4, 1.5 Hz, 1H), 6.86 (d, J = 8.2 Hz, 1H), 6.67 (d, J = 8.1 Hz, 1H), 2.65 (q, J = 7.5 Hz, 2H), 2.05 (s, 3H), 1.96 (s, 3H), 1.13 (t, J = 7.5 Hz, 3H). MS (ES⁺) m/z 368.1 (M + H)⁺. |
| | 2-((2-ethylphenyl)amino)-N-(3- | |
| | hydroxy-2,6-dimethylphenyl)thiazole- | |
| | 5-carboxamide | |
| BAA-485 | 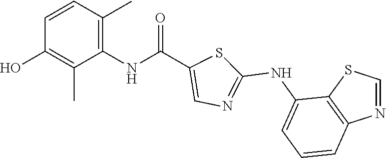 [SMILES: Cc1ccc(O)c(C)c1NC(=O)c1cnc(Nc2cccc3ncsc23)s1] | ¹H NMR (400 MHz, DMSO-d₆) δ 10.66 (s, 1H), 9.61 (s, 1H), 9.39 (s, 1H), 9.18 (s, 1H), 8.16-8.03 (m, 2H), 7.86 (d, J = 8.0 Hz, 1H), 7.57 (t, J = 8.0 Hz, 1H), 6.89 (d, J = 8.2 Hz, 1H), 6.69 (d, J = 8.2 Hz, 1H), 2.07 (s, 3H), 1.99 (s, 3H). MS (ES⁺) m/z 397.1 (M + H)⁺. |
| | 2-(benzo[d]thiazol-7-ylamino)-N-(3- | |
| | hydroxy-2,6-dimethylphenyl)thiazole- | |
| | 5-carboxamide | |
| BAA-486 | 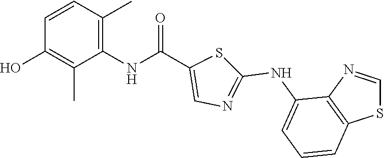 [SMILES: Cc1ccc(O)c(C)c1NC(=O)c1cnc(Nc2cccc3scnc23)s1] | ¹H NMR (400 MHz, DMSO-d₆) δ 11.01 (s, 1H), 9.63 (s, 1H), 9.41 (s, 1H), 9.18 (s, 1H), 8.57 (d, J = 7.9 Hz, 1H), 8.15 (s, 1H), 7.78 (d, J = 7.2 Hz, 1H), 7.48 (t, J = 8.0 Hz, 1H), 6.89 (d, J = 8.3 Hz, 1H), 6.69 (d, J = 8.2 Hz, 1H), 2.08 (s, 3H), 2.00 (s, 3H). MS (ES⁺) m/z 397.1 (M + H)⁺. |
| | 2-(benzo[d]thiazol-4-ylamino)-N-(3- | |
| | hydroxy-2,6-dimethylphenyl)thiazole- | |
| | 5-carboxamide | |
| BAA-487 | 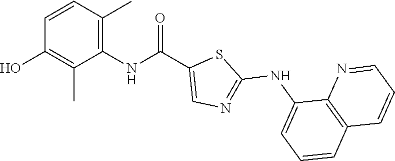 [SMILES: Cc1ccc(O)c(C)c1NC(=O)c1cnc(Nc2cccc3cccnc23)s1] | ¹H NMR (400 MHz, DMSO-d₆) δ 11.01 (s, 1H), 9.62 (s, 1H), 9.15 (s, 1H), 8.92 (dd, J = 4.2, 1.7 Hz, 1H), 8.80 (dd, J = 6.7, 2.3 Hz, 1H), 8.38 (dd, J = 8.3, 1.7 Hz, 1H), 8.16 (s, 1H), 7.69-7.52 (m, 3H), 6.86 (d, J = 8.1 Hz, 1H), 6.66 (d, J = 8.2 Hz, 1H), 2.05 (s, 3H), 1.97 (s, 3H). MS (ES⁺) m/z 391.2 (M + H)⁺. |
| | N-(3-hydroxy-2,6-dimethylphenyl)-2- | |
| | (quinolin-8-ylamino) thiazole-5- | |
| | carboxamide | |
| BAA-388 | 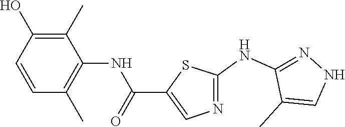 [SMILES: Cc1c[nH]nc1Nc1ncc(C(=O)Nc2c(C)ccc(O)c2C)s1] | MS (ES+) m/z 344 (M + H). ¹H NMR (300 MHz, MeOD) δ 8.00 (s, 1H), 7.41 (s, 1H), 6.91 (d, J = 8.2 Hz, 1H), 6.68 (d, J = 8.2 Hz, 1H), 2.15 (d, J = 0.8 Hz, 3H), 2.10 (s, 3H), 2.09-2.05 (m, 3H). |
| | N-(3-hydroxy-2,6-dimethylphenyl)-2- | |
| | ((4-methyl-1H-pyrazol-3- | |
| | yl)amino)thiazole-5-carboxamide | |
| BAA-541 | 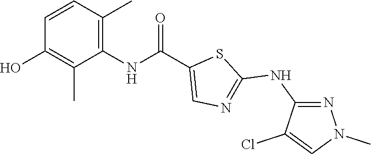 [SMILES: Cc1ccc(O)c(C)c1NC(=O)c1cnc(Nc2nn(C)cc2Cl)s1] | ¹H NMR (400 MHz, DMSO-d₆) δ 10.94 (s, 1H), 9.12 (s, 2H), 7.99 (s, 1H), 7.79 (s, 1H), 6.85 (d, J = 8.2 Hz, 1H), 6.66 (d, J = 8.1 Hz, 1H), 3.75 (s, 3H), 2.06 (s, 3H), 1.97 (s, 3H). MS (ES⁺) m/z 378.1/ 380.0 (M + H)⁺. |
| | 2-((4-chloro-1-methyl-1H-pyrazol-3- | |
| | yl)amino)-N-(3-hydroxy-2,6- | |
| | dimethylphenyl)thiazole-5- | |
| | carboxamide | |
| BAA-546 | 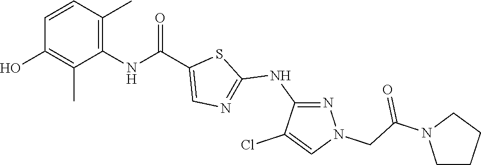 [SMILES: Cc1ccc(O)c(C)c1NC(=O)c1cnc(Nc2nn(CC(=O)N3CCCC3)cc2Cl)s1] | ¹H NMR (400 MHz, DMSO-d₆) δ 10.97 (s, 1H), 9.50 (s, 1H), 9.17 (s, 1H), 8.08 (s, 1H), 7.87 (s, 1H), 6.87 (d, J = 8.3 Hz, 1H), 6.67 (d, J = 8.2 Hz, 1H), 4.95 (s, 2H), 3.51 (t, J = 6.8 Hz, 2H), 3.30 (m, 2H), 2.06 (s, 3H), 1.97 (s, 3H), 1.91 (p, J = 6.8 Hz, 2H), 1.78 (p, J = 6.7 Hz, 2H). MS (ES⁺) m/z 475.1/477.1 (M + H)⁺. |
| | 2-((4-chloro-1-(2-oxo-2-(pyrrolidin-1- | |
| | yl)ethyl)-1H-pyrazol-3-yl)amino)-N-(3- | |
| | hydroxy-2,6-dimethylphenyl)thiazole- | |
| | 5-carboxamide | |
| BAA-560 | 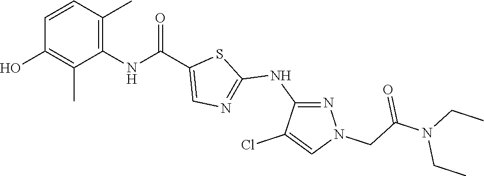 [SMILES: CCN(CC)C(=O)Cn1cc(Cl)c(Nc2ncc(C(=O)Nc3c(C)ccc(O)c3C)s2)n1] | ¹H NMR (400 MHz, DMSO-d₆) δ 10.96 (s, 1H), 9.47 (s, 1H), 9.14 (s, 1H), 8.05 (s, 1H), 7.86 (s, 1H), 6.84 (d, J = 8.2 Hz, 1H), 6.64 (d, J = 8.2 Hz, 1H), 4.98 (s, 2H), 3.35 (q, J = 7.1 Hz, 2H), 3.24 (q, J = 7.1 Hz, 2H), 2.02 (s, 3H), 1.94 (s, 3H), 1.13 (t, J = 7.1 Hz, 3H), 0.99 (t, J = 7.1 Hz, 3H). MS (ES⁺) m/z 477.2/479.2 (M + H)⁺. |
| | 2-((4-chloro-1-(2-(diethylamino)-2- | |
| | oxoethyl)-1H-pyrazol-3-yl)amino)-N- | |
| | (3-hydroxy-2,6-dimethylphenyl) | |
| | thiazole-5-carboxamide | |
| BAA-561 | 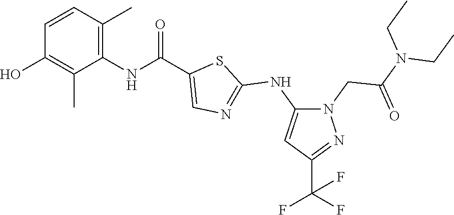 [SMILES: CCN(CC)C(=O)Cn1nc(C(F)(F)F)cc1Nc1ncc(C(=O)Nc2c(C)ccc(O)c2C)s1] | ¹H NMR (400 MHz, DMSO-d₆) δ 9.15 (s, 1H), 8.02 (s, 1H), 6.85 (d, J = 8.4 Hz, 1H), 6.65 (d, J = 8.2 Hz, 1H), 5.07 (s, 2H), 3.35 (q, J = 7.0 Hz, 2H), 3.25 (q, J = 7.2 Hz, 2H), 2.02 (s, 3H), 1.94 (s, 3H), 1.14 (t, J = 6.8 Hz, 3H), 1.00 (t, J = 7.0 Hz, 3H); MS (ES⁺) m/z 511.2 (M + H)⁺. |
| | 2-((1-(2-(diethylamino)-2-oxoethyl)-3- | |
| | (trifluoromethyl)-1H-pyrazol-5- | |
| | yl)amino)-N-(3-hydroxy-2,6- | |
| | dimethylphenyl)thiazole-5- | |
| | carboxamide | |
| BAA-569 | 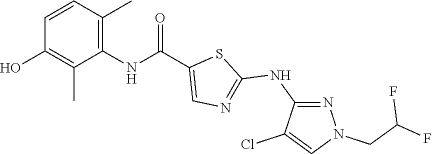 [SMILES: Cc1ccc(O)c(C)c1NC(=O)c1cnc(Nc2nn(CC(F)F)cc2Cl)s1] | ¹H NMR (400 MHz, DMSO-d₆) δ 9.47 (s, 1H), 9.16 (s, 1H), 8.08 (s, 1H), 7.99 (s, 1H), 6.87 (d, J = 8.2 Hz, 1H), 6.68 (d, J = 8.2 Hz, 1H), 6.35 (t, J = 54.8 Hz, 1H), 4.56 (t, J = 13.3 Hz, 2H), 2.06 (s, 3H), 1.98 (s, 3H). MS (ES⁺) m/z 428.2/430.1 (M + H)⁺. |
| | 2-((4-chloro-1-(2,2-difluoroethyl)-1H- | |
| | pyrazol-3-yl)amino)-N-(3-hydroxy-2,6- | |
| | dimethylphenyl)thiazole-5- | |
| | carboxamide | |
| BAA-566 | 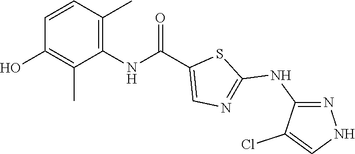 [SMILES: Cc1ccc(O)c(C)c1NC(=O)c1cnc(Nc2n[nH]cc2Cl)s1] | ¹H NMR (400 MHz, DMSO-d₆) δ 12.81 (s, 1H), 10.83 (s, 1H), 9.48 (s, 1H), 9.15 (s, 1H), 8.07 (s, 1H), 7.97 (s, 1H), 6.87 (d, J = 8.0 Hz, 1H), 6.67 (d, J = 8.4 Hz, 1H), 2.06 (s, 3H), 1.98 (s, 3H). MS (ES⁺) m/z 364.3/366.3 (M + H)⁺. |
| | 2-((4-chloro-1H-pyrazol-3-yl)amino)- | |
| | N-(3-hydroxy-2,6- | |
| | dimethylphenyl)thiazole-5- | |
| | carboxamide | |

### 2-((4-Bromo-1-(1,1-dioxidotetrahydrothiophen-3-yl)-5-methyl-1H-pyrazol-3-yl)amino)-N-(3-bromo-5-hydroxy-2.6-dimethylphenyl)thiazole-5-carboxamide( ) BAA-557

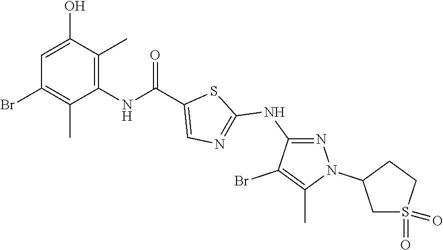 [SMILES: Cc1c(O)cc(Br)c(C)c1NC(=O)c1cnc(Nc2nn(C3CCS(=O)(=O)C3)c(C)c2Br)s1]

To a suspension of 2-[1-(1,1-dioxothiolan-3-yl)-5-methyl-pyrazol-3-yl]amino]-N-(3-methoxy-2,6-dimethyl-phenyl)thiazole-5-carboxamide (413 mg, 0.816 mmol) in DCM (50 mL) was added boron tribromide (6.5 mL, 6.53 mmol) and the resulting mixture was stirred at ambient temperature, under nitrogen for 16 h. The reaction was quenched with MeOH(20 mL), concentrated under reduced pressure, taken up in MeOH(20 mL), filtered through a syringe filter and concentrated under reduced pressure. The residue was triturated in MeCN then purified by preparative HPLC to provide the title compound (113 mg, 0.179 mmol, 21.88% yield) as a white solid.MS (ES-) m/z=618.0 [M-H]−. ¹H NMR (400 MHz, DMSO-D6) o 11.14 (s, 1H), 9.58 (s, 1H), 8.91 (s, 1H), 8.12 (s, 1H), 7.28 (s, 1H), 5.39-5.19 (m, 1H), 3.74 (dd, J=13.5, 8.3 Hz, 1H), 3.57 (dt, J=12.8, 6.3 Hz, 1H), 3.32-3.22 (m, 2H), 2.63-2.52 (m, 2H), 2.30 (s, 3H), 2.08 (s, 6H).

### N-(3-Bromo-5-hydroxy-2,6-dimethyl-phenyl)-2-[[1-(1,1-dioxothiolan-3-yl)-5-methyl-pyrazol-3-yl]amino]thiazole-5-carboxamide( ) BAA-563 2-[[1-(1, 1-Dioxothiolan-3-yl)-5-methyl-pyrazol-3-yl]amino]-N-(3-hydroxy-2,6-dimethyl-phenyl)thiazole-5-carboxamide( ) BAA-564

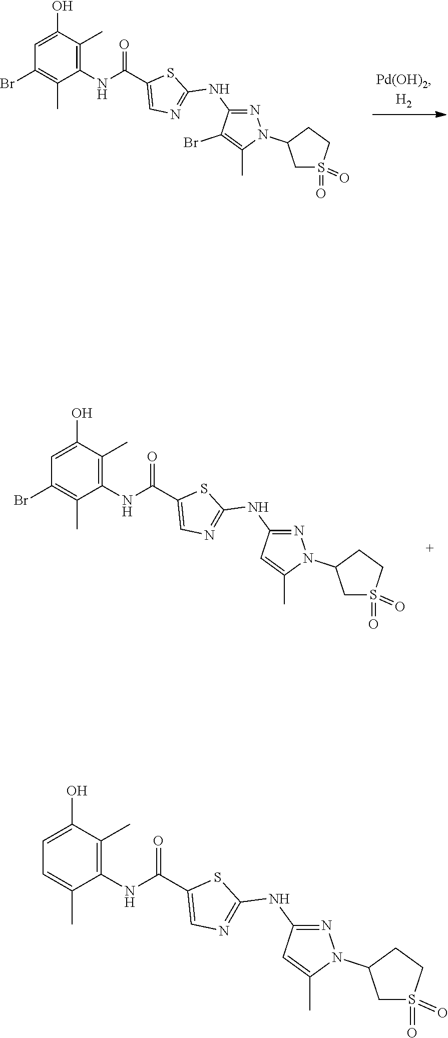 [SMILES: Cc1c(O)cc(Br)c(C)c1NC(=O)c1cnc(Nc2nn(C3CCS(=O)(=O)C3)c(C)c2Br)s1 ; Cc1c(O)cc(Br)c(C)c1NC(=O)c1cnc(Nc2cc(C)n(C3CCS(=O)(=O)C3)n2)s1 ; Cc1ccc(O)c(C)c1NC(=O)c1cnc(Nc2cc(C)n(C3CCS(=O)(=O)C3)n2)s1]

To a solution of 2-[4-bromo-1-(1,1-dioxothiolan-3-yl)-5-methyl-pyrazol-3-yl]amino]-N-(3-bromo-5-hydroxy-2,6-dimethyl-phenyl)thiazole-5-carboxamide (100 mg, 0.158 mmol) in MeCN (7.5 mL) was added Palladium hydroxide on carbon (Pd(OH) 2/C, 20% wt)(43 mg, 0.0614 mmol) in Water (3 mL) and the mixture degassed with nitrogen twice, then hydrogen twice. The reaction was stirred at rt under 1 atm of H2 for 3 days, then it was filtered through a short pad of Celite and the filtrate concentrated in vacuo. The crude product was purified by HPLC to afford 2-[1-(1,1-dioxothiolan-3-yl)-5-methyl-pyrazol-3-yl]amino]-N-(3-hydroxy-2,6-dimethyl-phenyl)thiazole-5-carboxamide (3.2 mg, 0.00626 mmol, 3.96% yield) and N-(3-bromo-5-hydroxy-2,6-dimethyl-phenyl)-2-[1-(1,1-dioxothiolan-3-yl)-5-methyl-pyrazol-3-yl]amino]thiazole-5-carboxamide (3.7 mg, 0.00618 mmol, 3.90% yield) as white solids.

BAA-563 analysis: MS (ES+) m/z=540.0/542.0 [M+H]⁺; ¹H NMR (400 MHz, DMSO-D6) δ 11.12 (s, 1H), 9.57 (s, 1H), 8.89 (s, 1H), 8.12 (s, 1H), 7.28 (s, 1H), 5.83 (s, 1H), 5.17 (t, J=7.9 Hz, 1H), 3.72 (dd, J=13.4, 8.3 Hz, 1H), 3.57 (dt, J=12.7, 6.3 Hz, 1H), 3.31-3.22 (m, 2H), 2.56 (d, J=7.9 Hz, 2H), 2.29 (s, 3H), 2.09 (s, 6H).

BAA-564 analysis: MS (ES+) m/z=462.2 [M+H]⁺; ¹H NMR (400 MHz, METHANOL-D4) § 8.04 (s, 1H), 6.92 (dt, J=8.3, 0.7 Hz, 1H), 6.69 (d, J=8.2 Hz, 1H), 5.84 (d, J=0.8 Hz, 1H), 3.73-3.42 (m, 4H), 3.29-3.19 (m, 1H), 2.86-2.56 (m, 2H), 2.34 (d, J=0.7 Hz, 3H), 2.17 (d, J=0.7 Hz, 3H), 2.12 (s, 3H).

### 2-((4-Acetamidophenyl)amino)-N-(3-hydroxy-2,6-dimethylphenyl)thiazole-5-carboxamide( ) BAA-513

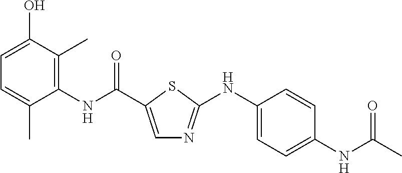 [SMILES: CC(=O)Nc1ccc(Nc2ncc(C(=O)Nc3c(C)ccc(O)c3C)s2)cc1]

Boron tribromide 1M in DCM (1.0 mL, 1.00 mmol) was added dropwise to a suspension of 2-(4-acetamidoanilino)-N-(3-methoxy-2,6-dimethyl-phenyl)thiazole-5-carboxamide (60 mg, 0.146 mmol) in DCM (3 mL) under inert atmosphere (N2). The reaction was stirred at rt overnight then it was quenched by careful addition of MeOH(2 mL), and the solvent was evaporated. The residue was purified by column chromatography over C18 (23 g cartridge) eluting with a gradient of MeCN
(0.1% NH₃)(5% to 95%; v/v) in water (0.1% NH₃) to afford 2-(4-acetamidoanilino)-N-(4-bromo-3-hydroxy-2,6-dimethyl-phenyl)thiazole-5-carboxamide (60 mg, 0.110 mmol, 75.36% yield) as a brown solid.MS (ES+) m/z=475.1, 477.0 (mono Br pattern). ¹H NMR (400 MHz, DMSO-d₆) δ 10.51 (s, 1H), 9.89 (s, 1H), 9.64 (s, 1H), 8.88 (s, 1H), 8.07 (s, 1H), 7.54 (s, 4H), 7.28 (s, 1H), 2.08 (s, 6H), 2.02 (s, 3H).

The product from the first step was dissolved in MeCN (5 mL) and the mixture was degassed by performing vacuum/N2 cycles twice. Palladium hydroxide on carbon (Pd(OH) 2/C, 20% wt)(30 mg, 0.0427 mmol) was suspended in water (2 mL) and added to the reaction mixture, which was stirred at rt under 1 atm of H2 for 16 h. The reaction mixture was filtered through a short pad of Celite, the filtrate was concentrated in vacuo, and the crude was purified by column chromatography over C18 (23 g cartridge) eluting with a gradient of MeCN (0.1% NH₃)(5% to 95%; v/v) in water (0.1% NH₃) and liophilised to afford the title compound (18 mg, 0.0448 mmol, 40.65% yield) as a white solid. MS (ES+) m/z=397.2 [M+H]⁺, ¹H NMR (400 MHz, DMSO-d₆) o 10.47 (s, 1H), 9.88 (s, 1H), 9.54 (s, 1H), 9.17 (s, 1H), 8.06 (s, 1H), 7.53 (s, 4H), 6.88 (d, J=8.2 Hz, 1H), 6.68 (d, J=8.1 Hz, 1H), 2.06 (s, 3H), 2.02 (s, 3H), 1.98 (s, 3H).

### 2-((1-(2-Bromoethyl)-4-chloro-1H-pyrazol-3-yl)amino)-N-(3-hydroxy-2,6-dimethylphenyl)thiazole-5-carboxamide( ) BAA-574

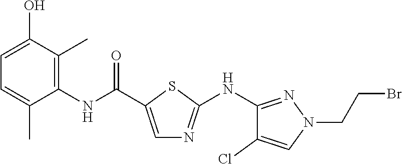 [SMILES: Cc1ccc(O)c(C)c1NC(=O)c1cnc(Nc2nn(CCBr)cc2Cl)s1]

4-Chloro-1-(2-fluoroethyl)pyrazol-3-amine (140 mg, 0.856 mmol) and 2-bromo-N-(3-methoxy-2,6-dimethyl-phenyl)thiazole-5-carboxamide (150 mg, 0.440 mmol) were dissolved in 1,4-dioxane (3 mL). Xantphos (50 mg, 0.0864 mmol) and potassium carbonate 325 mesh (180 mg, 1.30 mmol) were added, the mixture was degassed with nitrogen for 5 minutes and palladium (II) acetate (10 mg, 0.0445 mmol) was subsequently added. The reaction was stirred at 70° C. overnight in a sealed vial, then the mixture was filtered through celite and rinsed with MeOH. The solution was concentrated and the crude material was purified by column chromatography over C18 (23 g cartridge) eluting with a gradient of MeCN (0.1% NH₃)(5% to 80%; v/v) in water (0.1% NH₃), affording 2-[4-chloro-1-(2-fluoroethyl)pyrazol-3-yl]amino]-N-(3-methoxy-2,6-dimethyl-phenyl)thiazole-5-carboxamide (97 mg, 0.199 mmol, 45.24% yield) as a pale yellow solid. MS (ES+) m/z=424.2/426.1 (mono CI pattern) [M+H]⁺, ¹H NMR (400 MHz, DMSO-d₆) δ 9.58 (s, 1H), 8.10 (s, 1H), 8.01 (s, 1H), 7.06 (d, J=8.5 Hz, 1H), 6.84 (d, J=8.5 Hz, 1H), 4.78 (dt, J=47.2, 4.6 Hz, 2H), 4.37 (dt, J=27.5, 4.6 Hz, 2H), 3.77 (s, 3H), 2.11 (s, 3H), 2.01 (s, 3H).

The methoxy product from the first step (90 mg, 0.212 mmol) was suspended in DCM (3.5 mL) and boron tribromide (1.3 mL, 1.30 mmol) was added under inert atmosphere (N2). The reaction was stirred at rt for 3h, quenched by addition of MeOH(15 mL), stirred overnight. MS analysis suggested that the fluorine has been exchanged with a bromine. The solvent was evaporated in vacuo, the crude solid was purified by prep HPLC to afford 2-[1-(2-bromoethyl)-4-chloro-pyrazol-3-yl]amino]-N-(3-hydroxy-2,6-dimethyl-phenyl)thiazole-5-carboxamide (15 mg, 0.0327 mmol, 15.41% yield) as a white solid. MS (ES+) m/z=470.2/472.2/474.2 (mono Cl and mono Br pattern) [M+H]⁺, ¹H NMR (400 MHz, DMSO-d₆) δ 11.00 (s, 1H), 9.51 (s, 1H), 9.17 (s, 1H), 8.09 (s, 1H), 8.04 (s, 1H), 6.88 (d, J=8.2 Hz, 1H), 6.68 (d, J=8.2 Hz, 1H), 4.45 (t, J=5.8 Hz, 2H), 3.88 (t, J=5.8 Hz, 2H), 2.06 (s, 3H), 1.98 (s, 3H).

### Synthesis Using TBDMS-Protected Phenol

### 3-[tert-Butyl(dimethyl) silyl]oxy-2,6-dimethyl-aniline

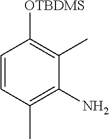 [SMILES: Cc1ccc(O[Si](C)(C)C(C)(C)C)c(C)c1N]

tert-Butyldimethylchlorosilane (2.00 eq, 17.32 g, 115 mmol) was added to a mixture of 3-amino-2,4-dimethyl-phenol (1.00 eq, 7.88 g, 57.4 mmol) and Imidazole (4.03 eq, 15.75 g, 231 mmol) in anhydrous THF (200 mL) at r.t. The reaction mixture was stirred at r.t. for 3.5 h. The reaction mixture was concentrated under reduced pressure and the residue taken up in a mixture of EtOAc (250 mL) and H₂O (250 mL). The layers were separated and the organic layer was washed with H₂O (2×200 mL) and brine (200 mL), before being dried over Na₂SO₄, filtered and the filtrates concentrated under reduced pressure to afford a brown oil which was purified by column chromatography over silica (330 g cartridge) eluting with a gradient of EtOAc (0% to 20%; v/v) in iso-Hexane [Note 4] to afford the desired product 3-[tert-butyl(dimethyl) silyl]oxy-2,6-dimethyl-aniline (15.80 g, 41.5 mmol, 72.20% yield) as a yellow oil. MS (ES+) m/z=252.3 [M+H]+. ¹H NMR (400 MHz, CDCl₃) δ 6.77 (dt, J=8.0, 0.8 Hz, 1H), 6.24 (d, J=8.1 Hz, 1H), 3.57 (br s, 2H), 2.12 (d, J=0.8 Hz, 3H), 2.06 (s, 3H), 1.02 (s, 9H), 0.20 (s, 6H).

### 2-Bromo-N-[3-[tert-butyl(dimethyl) silyl]oxy-2,6-dimethyl-phenyl]thiazole-5-carboxamide

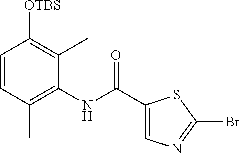 [SMILES: Cc1ccc(O[Si](C)(C)C(C)(C)C)c(C)c1NC(=O)c1cnc(Br)s1]

3-[tert-Butyl(dimethyl) silyl]oxy-2,6-dimethyl-aniline (1.00 eq, 15.80 g, 41.5 mmol) was partially dissolved in MeCN (250 mL) at r.t., to which 2-bromothiazole-5-carboxylic acid (1.50 eq, 12.94 g, 62.2 mmol), DIPEA (2.00 eq, 14 mL, 82.9 mmol) and 1-propanephosphonic anhydride, 50 wt % in EtOAc (1.50 eq, 37 mL, 62.2 mmol) were added sequentially. The reaction mixture was heated at r.t for 3.5 h. Additional DIPEA (1.02 eq, 7.3 mL, 42.1 mmol) and 1-propanephosphonic anhydride (0.306 eq, 7.3 mL, 12.7 mmol) were added and the reaction mixture was heated to 65° C. for 18 h. The reaction mixture was diluted with H₂O (200 mL) and the volatiles removed under reduced pressure before being poured into a mixture of EtOAc (300 mL) and H₂O (150 mL). The layers were separated and the aqueous layer was extracted with EtOAc (2×100 mL). The combined organic layers were washed with H₂O (200 mL) and brine (200 mL), before being dried over Na₂SO₄, filtered and the filtrates concentrated under reduced pressure. The crude material was purified by column chromatography over silica (330 g cartridge) eluting with a gradient of EtOAc (0% to 20%; v/v) in iso-Hexane, to afford the title compound (13.48 g, 30.5 mmol, 73.63% yield) as a pale-yellow foamy solid. ES (MS+) m/z=443.0/441.0 [M+H]+ and 441.1/439.1 [M-H]−, mono-Br splitting pattern. ¹H NMR (400 MHz, CDCl₃) δ 8.05 (s, 1H), 7.23 (br s, 1H), 6.96 (d, J=8.3 Hz, 1H), 6.72 (d, J=8.3 Hz, 1H), 2.18 (s, 3H), 2.12 (s, 3H), 1.01 (s, 9H), 0.20 (s, 6H).

General Method X: Palladium Catalysed Coupling of 2-Bromo-N-(3-Hydroxy-2,6-Dimethyl-Phenyl)Thiazole-5-Carboxamide with Amines and Silyl Deprotection

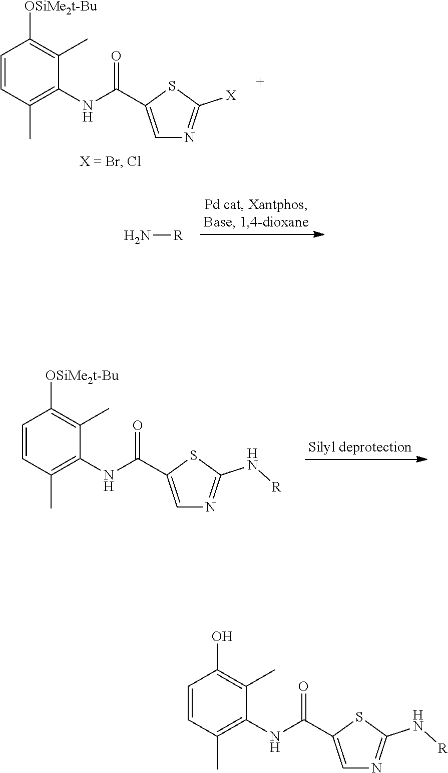

A mixture of bromothiazole (1.0 eq), substituted amine (2-4 eq), cesium carbonate or potassium carbonate (2-3 eq), Pd(OAc) 2 (0.1 eq), Xantphos (0.2) and 1,4-dioxane was degassed with nitrogen and stirred at reflux for 6-24 h. After cooling to rt, the mixture was filtered through celite and evaporated under reduced pressure. The crude was purified by chromatography over silica.

The silylated intermediate was dissolved in DCM and concentrated HCl was added. The mixture was stirred at 40° C. for 18-72 h, concentrated under reduced pressure, and the crude purified by reverse column chromatography using a MeCN (0.1% formic acid): water (0.1% formic acid) gradient to provide the desired phenol product.

### 2-((5-Bromo-1-methyl-1H-pyrazol-3-yl)amino)-N-(3-hydroxy-2,6-dimethylphenyl)thiazole-5-carboxamide( ) BAA-494

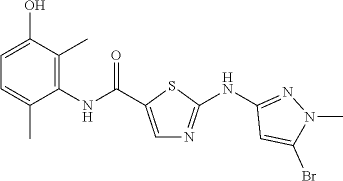 [SMILES: Cc1ccc(O)c(C)c1NC(=O)c1cnc(Nc2cc(Br)n(C)n2)s1]

Prepared as described in Method X from 2-bromo-N-[3-[tert-butyl(dimethyl) silyl]oxy-2,6-dimethyl-phenyl]thiazole-5-carboxamide (1.00 eq, 150 mg, 0.340 mmol), 5-bromo-1-methyl-1H-pyrazol-3-amine (3.00 eq, 179 mg, 1.02 mmol), palladium (II)acetate (0.100 eq, 7.6 mg, 0.0340 mmol), 4,5-Bis(diphenylphosphino)-9,9-dimethylxanthene (Xantphos)(0.200 eq, 39 mg, 0.0680 mmol) and potassium carbonate 325 mesh (2.50 eq, 117 mg, 0.849 mmol) in anhydrous 1,4-dioxane (3.0 mL), to afford 2-[(5-bromo-1-methyl-pyrazol-3-yl)amino]-N-[3-[tert-butyl(dimethyl) silyl]oxy-2,6-dimethyl-phenyl]thiazole-5-carboxamide (128 mg, 0.215 mmol, 63.19% yield) after purification by column chromatography over silica (40 g cartridge) eluting with a gradient of EtOAc (0% to 100%; v/v) in iso-Hexane. MS (ES+) m/z=538.0/536.1 [M+H]+, mono-Br splitting pattern. ¹H NMR (400 MHz, CDCl₃) δ 10.13 (br s, 1H), 7.92 (s, 1H), 7.01 (s, 1H), 6.95 (d, J=8.4 Hz, 1H), 6.70 (d, J=8.2 Hz, 1H), 6.05 (s, 1H), 3.85 (s, 3H), 2.22 (s, 3H), 2.15 (s, 3H), 1.00 (s, 9H), 0.21 (s, 6H).

This silylated intermediate (1.00 eq, 125 mg, 0.233 mmol) was dissolved in DCM (2.0 mL) at r.t., to which conc. hydrogen chloride solution (151 eq, 3.0 mL, 35.1 mmol) was added. The reaction mixture was heated at 40° C. for 18 h, then it was concentrated under reduced pressure. The crude material was purified by column chromatography over C18 (12 g cartridge) eluting with a gradient of MeCN (0.1% formic acid)(5% to 95%; v/v) in water (0.1% formic acid) to afford the desired product 2-[(5-bromo-1-methyl-pyrazol-3-yl)amino]-N-(3-hydroxy-2,6-dimethyl-phenyl)thiazole-5-carboxamide (28 mg, 0.0630 mmol, 27.04% yield) as a white solid. MS (ES+) m/z=422.2/424.4 [M+H]+, mono-Br splitting pattern. ¹H NMR (400 MHz, DMSO-d₆) δ 11.12 (s, 1H), 9.53 (s, 1H), 9.17 (s, 1H), 8.10 (s, 1H), 6.88 (d, J=8.2 Hz, 1H), 6.68 (d, J=8.2 Hz, 1H), 6.17 (s, 1H), 3.77 (s, 3H), 2.06 (s, 3H), 1.98 (s, 3H).

The following example compounds were prepared similarly using Method X with appropriately substituted aromatic and heteroaromatic amines.

| # | Structure & Name | Analytical Data |
|---|---|---|
| BAA-470 | 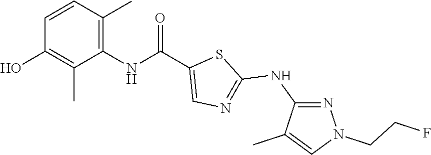 [SMILES: Cc1cn(CCF)nc1Nc1ncc(C(=O)Nc2c(C)ccc(O)c2C)s1] | ¹H NMR (400 MHz, DMSO-d₆) δ 10.73 (s, 1H), 9.46 (s, 1H), 9.16 (s, 1H), 8.10 (s, 1H), 7.50 (s, 1H), 6.88 (d, J = 8.2 Hz, 1H), 6.67 (d, J = 8.2 Hz, 1H), 4.81 (t, J = 4.7 Hz, 1H), 4.69 (t, J = 4.7 Hz, 1H), 4.34 (t, J = 4.7 Hz, 1H), 4.27 (t, J = 4.7 Hz, 1H), 2.06 (s, 3H), 2.00 (s, 3H), 1.98 (s, 3H). MS (ES⁺) m/z 390.1 (M + H)⁺. |
| | 2-((1-(2-fluoroethyl)-4-methyl-1H- | |
| | pyrazol-3-yl)amino)-N-(3-hydroxy-2,6- | |
| | dimethylphenyl)thiazole-5- | |
| | carboxamide | |
| BAA-473 | 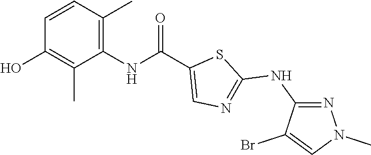 [SMILES: Cc1ccc(O)c(C)c1NC(=O)c1cnc(Nc2nn(C)cc2Br)s1] | ¹H NMR (400 MHz, DMSO-d₆) δ 9.49 (s, 1H), 9.17 (s, 1H), 8.06 (s, 1H), 7.91 (s, 1H), 6.87 (d, J = 8.2 Hz, 1H), 6.67 (d, J = 8.2 Hz, 1H), 3.81 (s, 3H), 2.06 (s, 3H), 1.98 (s, 3H). MS (ES⁺) m/z 422.2/424.2 (M + H)⁺. |
| | 2-((4-bromo-1-methyl-1H-pyrazol-3- | |
| | yl)amino)-N-(3-hydroxy-2,6- | |
| | dimethylphenyl)thiazole-5- | |
| | carboxamide | |
| BAA-477 | 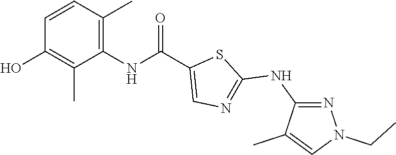 [SMILES: CCn1cc(C)c(Nc2ncc(C(=O)Nc3c(C)ccc(O)c3C)s2)n1] | ¹H NMR (400 MHz, DMSO-d₆) δ 10.64 (br s, 1H), 9.44 (s, 1H), 9.16 (s, 1H), 8.09 (s, 1H), 7.46 (d, J = 1.0 Hz, 1H), 6.87 (d, J = 8.2 Hz, 1H), 6.68 (d, J = 8.2 Hz, 1H), 4.01 (q, J = 7.2 Hz, 2H), 2.06 (s, 3H), 1.99-1.97 (m, 6H), 1.35 (t, J = 7.2 Hz, 3H). MS (ES⁺) m/z 372.3 (M + H)⁺. |
| | 2-((1-ethyl-4-methyl-1H-pyrazol-3- | |
| | yl)amino)-N-(3-hydroxy-2,6- | |
| | dimethylphenyl)thiazole-5- | |
| | carboxamide | |
| BAA-478 | 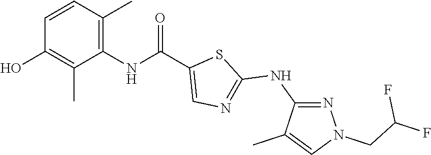 [SMILES: Cc1cn(CC(F)F)nc1Nc1ncc(C(=O)Nc2c(C)ccc(O)c2C)s1] | ¹H NMR (400 MHz, DMSO-d₆) δ 10.77 (s, 1H), 9.47 (s, 1H), 9.17 (s, 1H), 8.11 (s, 1H), 7.52 (d, J = 1.0 Hz, 1H), 6.88 (d, J = 8.2 Hz, 1H), 6.68 (d, J = 8.2 Hz, 1H), 6.31 (tt, J = 55.1, 3.8 Hz, 1H), 4.50 (td, J = 14.9, 3.8 Hz, 2H), 2.07 (s, 3H), 2.01 (d, J = 0.8 Hz, 3H), 1.98 (s, 3H). MS (ES⁺) m/z 408.3 (M + H)⁺. |
| | 2-((1-(2,2-difluoroethyl)-4-methyl-1H- | |
| | pyrazol-3-yl)amino)-N-(3-hydroxy-2,6- | |
| | dimethylphenyl)thiazole-5- | |
| | carboxamide | |
| BAA-490 | 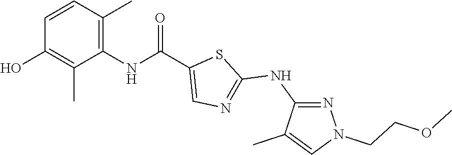 [SMILES: COCCn1cc(C)c(Nc2ncc(C(=O)Nc3c(C)ccc(O)c3C)s2)n1] | ¹H NMR (400 MHz, DMSO-d₆) δ 10.58 (br s, 1H), 9.45 (s, 1H), 9.17 (s, 1H), 8.09 (s, 1H), 7.44 (d, J = 0.9 Hz, 1H), 6.87 (d, J = 8.2 Hz, 1H), 6.68 (d, J = 8.2 Hz, 1H), 4.13 (t, J = 5.3 Hz, 2H), 3.67 (t, J = 5.3 Hz, 2H), 3.22 (s, 3H), 2.06 (s, 3H), 1.98 (s, 6H). MS (ES⁺) m/z 402.3 (M + H)⁺. |
| | N-(3-hydroxy-2,6-dimethylphenyl)-2- | |
| | ((1-(2-methoxyethyl)-4-methyl-1H- | |
| | pyazol-3-yl)amino)thiazole-5- | |
| | carboxamide | |
| BAA-495 | 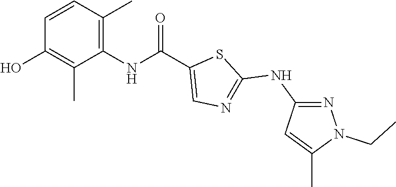 [SMILES: CCn1nc(Nc2ncc(C(=O)Nc3c(C)ccc(O)c3C)s2)cc1C] | ¹H NMR (400 MHz, DMSO-d₆) δ 10.92 (s, 1H), 9.46 (s, 1H), 9.16 (s, 1H), 8.07 (s, 1H), 6.88 (d, J = 8.2 Hz, 1H), 6.68 (d, J = 8.2 Hz, 1H), 5.76 (d, J = 0.8 Hz, 1H), 3.98 (q, J = 7.2 Hz, 2H), 2.23 (d, J = 0.7 Hz, 3H), 2.06 (s, 3H), 1.98 (s, 3H), 1.31 (t, J = 7.2 Hz, 3H). MS (ES⁺) m/z 372.3 (M + H)⁺. |
| | 2-((1-ethyl-5-methyl-1H-pyrazol-3- | |
| | yl)amino)-N-(3-hydroxy-2,6- | |
| | dimethylphenyl)thiazole-5- | |
| | carboxamide | |
| BAA-499 | 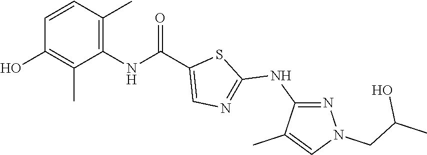 [SMILES: Cc1cn(CC(C)O)nc1Nc1ncc(C(=O)Nc2c(C)ccc(O)c2C)s1] | ¹H NMR (400 MHz, DMSO-d₆) δ 8.09 (s, 1H), 7.41 (d, J = 1.0 Hz, 1H), 6.87 (d, J = 8.2 Hz, 1H), 6.67 (d, J = 8.2 Hz, 1H), 3.97 (q, J = 6.1 Hz, 1H), 3.87 (dd, J = 6.0, 1.2 Hz, 2H), 2.05 (s, 3H), 1.98- 1.95 (m, 6H), 1.03 (d, J = 6.2 Hz, 3H). MS (ES⁺) m/z 402.4 (M + H)⁺. |
| | N-(3-hydroxy-2,6-dimethylphenyl)-2- | |
| | ((1-(2-hydroxypropyl)-4-methyl-1H- | |
| | pyrazol-3-yl)amino)thiazole-5- | |
| | carboxamide | |
| BAA-501 | 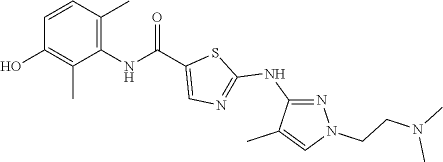 [SMILES: Cc1cn(CCN(C)C)nc1Nc1ncc(C(=O)Nc2c(C)ccc(O)c2C)s1] | ¹H NMR (400 MHz, DMSO-d₆) δ 10.88 (s, 1H), 9.74 (s, 1H), 9.51 (s, 1H), 9.19 (s, 1H), 8.15 (s, 1H), 7.58 (s, 1H), 6.88 (d, J = 8.2 Hz, 1H), 6.68 (d, J = 8.2 Hz, 1H), 4.43 (t, J = 6.1 Hz, 2H), 3.53 (q, J = 5.9 Hz, 2H), 2.79 (d, J = 4.8 Hz, 6H), 2.06 (s, 3H), 2.02 (s, 3H), 1.98 (s, 3H). MS (ES⁺) m/z 415.3 (M + H)⁺. |
| | 2-((1-(2-(dimethylamino)ethyl)-4- | |
| | methyl-1H-pyrazol-3-yl)amino)-N-(3- | |
| | hydroxy-2,6-dimethylphenyl)thiazole- | |
| | 5-carboxamide | |
| BAA-503 | 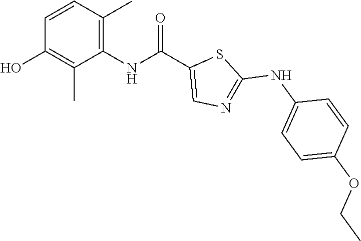 [SMILES: CCOc1ccc(Nc2ncc(C(=O)Nc3c(C)ccc(O)c3C)s2)cc1] | ¹H NMR (400 MHz, DMSO-d₆) δ 10.36 (s, 1H), 9.52 (s, 1H), 9.17 (s, 1H), 8.04 (s, 1H), 7.51 (d, J = 9.0 Hz, 2H), 6.92 (d, J = 9.1 Hz, 2H), 6.88 (d, J = 8.2 Hz, 1H), 6.68 (d, J = 8.2 Hz, 1H), 3.99 (q, J = 7.0 Hz, 2H), 2.06 (s, 3H), 1.98 (s, 3H), 1.32 (t, J = 7.0 Hz, 3H). MS (ES⁺) m/z 384.2 (M + H)⁺. |
| | 2-((4-ethoxyphenyl)amino)-N-(3- | |
| | hydroxy-2,6-dimethylphenyl)thiazole- | |
| | 5-carboxamide | |
| BAA-515 | 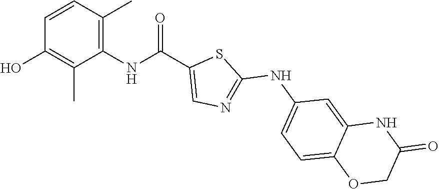 [SMILES: Cc1ccc(O)c(C)c1NC(=O)c1cnc(Nc2ccc3c(c2)NC(=O)CO3)s1] | ¹H NMR (400 MHz, DMSO-d₆) δ 10.61 (s, 2H), 9.38 (s, 1H), 9.12 (s, 1H), 8.00 (s, 1H), 7.26 (s, 1H), 7.01 (s, 1H), 6.87 (dd, J = 8.4, 3.5 Hz, 2H), 6.67 (d, J = 8.1 Hz, 1H), 4.50 (s, 2H), 2.06 (s, 3H), 1.98 (s, 3H). MS (ES⁺) m/z 411.1 (M + H)⁺. |
| | N-(3-hydroxy-2,6-dimethylphenyl)-2- | |
| | ((3-oxo-3,4-dihydro-2H- | |
| | benzo[b][1,4]oxazin-6- | |
| | yl)amino)thiazole-5-carboxamide | |
| BAA-520 | 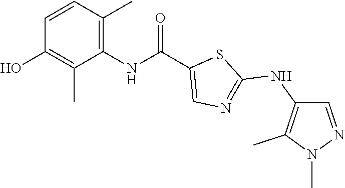 [SMILES: Cc1ccc(O)c(C)c1NC(=O)c1cnc(Nc2cnn(C)c2C)s1] | ¹H NMR (400 MHz, DMSO-d₆) δ 9.52 (s, 1H), 9.40 (s, 1H), 9.22 (s, 1H), 7.95 (s, 1H), 7.51 (s, 1H), 6.85 (d, J = 8.2 Hz, 1H), 6.67 (d, J = 8.2 Hz, 1H), 3.73 (s, 3H), 2.17 (s, 3H), 2.04 (s, 3H), 1.95 (s, 3H). MS (ES⁺) m/z 358.3 (M + H)⁺. |
| | 2-((1,5-dimethyl-1H-pyrazol-4- | |
| | yl)amino)-N-(3-hydroxy-2,6- | |
| | dimethylphenyl)thiazole-5- | |
| | carboxamide | |
| BAA-521 | 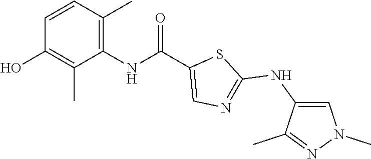 [SMILES: Cc1ccc(O)c(C)c1NC(=O)c1cnc(Nc2cn(C)nc2C)s1] | ¹H NMR (400 MHz, DMSO-d₆) δ 9.19 (s, 1H), 7.91 (s, 1H), 7.80 (s, 1H), 6.85 (d, J = 8.2 Hz, 1H), 6.66 (d, J = 8.2 Hz, 1H), 3.73 (s, 3H), 2.08 (s, 3H), 2.04 (s, 3H), 1.96 (s, 3H). MS (ES⁺) m/z 358.3 (M + H)⁺. |
| | 2-((1,3-dimethyl-1H-pyrazol-4- | |
| | yl)amino)-N-(3-hydroxy-2,6- | |
| | dimethylphenyl)thiazole-5- | |
| | carboxamide | |
| BAA-525 | 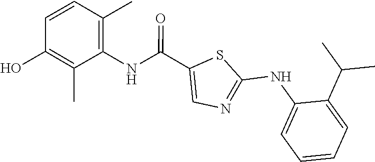 [SMILES: Cc1ccc(O)c(C)c1NC(=O)c1cnc(Nc2ccccc2C(C)C)s1] | ¹H NMR (400 MHz, DMSO-d₆) δ 9.83 (s, 1H), 9.44 (s, 1H), 9.16 (s, 1H), 7.97 (s, 1H), 7.54-7.46 (m, 1H), 7.43-7.34 (m, 1H), 7.31-7.19 (m, 2H), 6.86 (d, J = 8.2 Hz, 1H), 6.66 (d, J = 8.2 Hz, 1H), 3.24 (p, J = 6.8 Hz, 1H), 2.04 (s, 3H), 1.96 (s, 3H), 1.16 (d, J = 6.8 Hz, 6H). MS (ES⁺) m/z 382.4 (M + H)⁺. |
| | N-(3-hydroxy-2,6-dimethylphenyl)-2- | |
| | ((2-isopropylphenyl)amino)thiazole-5- | |
| | carboxamide | |
| BAA-526 | 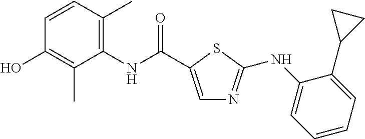 [SMILES: Cc1ccc(O)c(C)c1NC(=O)c1cnc(Nc2ccccc2C2CC2)s1] | ¹H NMR (400 MHz, DMSO-d₆) δ 9.91 (s, 1H), 9.48 (s, 1H), 9.15 (s, 1H), 8.00 (s, 1H), 7.65 (d, J = 7.9 Hz, 1H), 7.20 (td, J = 7.6, 1.6 Hz, 1H), 7.12 (td, J = 7.6, 1.3 Hz, 1H), 6.99 (dd, J = 7.8, 1.6 Hz, 1H), 6.87 (d, J = 8.2 Hz, 1H), 6.67 (d, J = 8.2 Hz, 1H), 2.08-2.00 (m, 4H), 1.97 (s, 3H), 0.99-0.89 (m, 2H), 0.68-0.59 (m, 2H). MS (ES⁺) m/z 380.3 (M + H)⁺. |
| | 2-((2-cyclopropylphenyl)amino)-N-(3- | |
| | hydroxy-2,6-dimethylphenyl)thiazole- | |
| | 5-carboxamide | |
| BAA-527 | 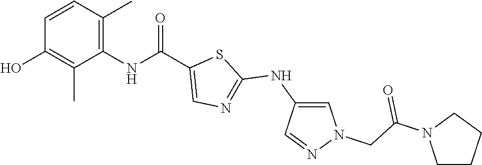 [SMILES: Cc1ccc(O)c(C)c1NC(=O)c1cnc(Nc2cnn(CC(=O)N3CCCC3)c2)s1] | ¹H NMR (400 MHz, DMSO-d₆) δ 10.35 (s, 1H), 9.51 (s, 1H), 8.03 (s, 1H), 7.93 (d, J = 0.8 Hz, 1H), 7.46 (d, J = 0.8 Hz, 1H), 6.87 (d, J = 8.2 Hz, 1H), 6.68 (d, J = 8.2 Hz, 1H), 5.00 (s, 2H), 3.46 (t, J = 6.8 Hz, 2H), 3.31 (t, J = 6.9 Hz, 2H), 2.06 (s, 3H), 1.97 (s, 3H), 1.91 (p, J = 6.8 Hz, 2H), 1.78 (p, J = 6.7 Hz, 2H). MS (ES⁺) m/z 441.4 (M + H)⁺. |
| | N-(3-hydroxy-2,6-dimethylphenyl)-2- | |
| | ((1-(2-oxo-2-(pyrrolidin-1-yl)ethyl)-1H- | |
| | pyrazol-4-yl)amino)thiazole-5- | |
| | carboxamide | |
| BAA-528 | 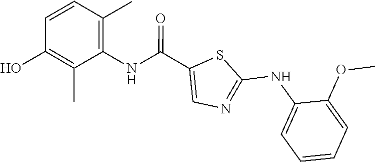 [SMILES: COc1ccccc1Nc1ncc(C(=O)Nc2c(C)ccc(O)c2C)s1] | ¹H NMR (400 MHz, DMSO-d₆) δ 9.88 (s, 1H), 9.53 (s, 1H), 9.17 (s, 1H), 8.23 (d, J = 7.3 Hz, 1H), 8.04 (s, 1H), 7.11-6.99 (m, 2H), 6.95 (ddd, J = 8.4, 6.3, 2.6 Hz, 1H), 6.88 (d, J = 8.2 Hz, 1H), 6.68 (d, J = 8.2 Hz, 1H), 3.86 (s, 3H), 2.06 (s, 3H), 1.98 (s, 3H). MS (ES⁺) m/z 370.3 (M + H)⁺. |
| | N-(3-hydroxy-2,6-dimethylphenyl)-2- | |
| | ((2-methoxyphenyl)amino)thiazole-5- | |
| | carboxamide. TFA salt | |
| BAA-529 | 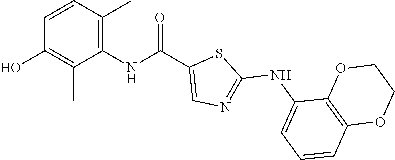 [SMILES: Cc1ccc(O)c(C)c1NC(=O)c1cnc(Nc2cccc3c2OCCO3)s1] | ¹H NMR (400 MHz, DMSO-d₆) δ 9.90 (s, 1H), 9.53 (s, 1H), 9.16 (s, 1H), 8.04 (s, 1H), 7.77 (d, J = 6.9 Hz, 1H), 6.88 (d, J = 8.2 Hz, 1H), 6.80 (t, J = 8.2 Hz, 1H), 6.68 (d, J = 8.2 Hz, 1H), 6.59 (dd, J = 8.2, 1.5 Hz, 1H), 4.38-4.21 (m, 4H), 2.06 (s, 3H), 1.98 (s, 3H). MS (ES⁺) m/z 398.3 (M + H)⁺. |
| | 2-((2,3-dihydrobenzo[b][1,4]dioxin-5- | |
| | yl)amino)-N-(3-hydroxy-2,6- | |
| | dimethylphenyl)thiazole-5- | |
| | carboxamide. TFA salt | |
| BAA-530 | 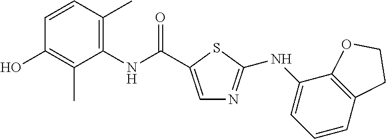 [SMILES: Cc1ccc(O)c(C)c1NC(=O)c1cnc(Nc2cccc3c2OCC3)s1] | ¹H NMR (400 MHz, DMSO-d₆) δ 10.03 (s, 1H), 9.51 (s, 1H), 9.16 (s, 1H), 8.02 (s, 1H), 7.85 (d, J = 8.0 Hz, 1H), 6.98 (d, J = 6.3 Hz, 1H), 6.87 (d, J = 8.2 Hz, 1H), 6.83 (dd, J = 8.1, 7.3 Hz, 1H), 6.67 (d, J = 8.2 Hz, 1H), 4.61 (t, J = 8.7 Hz, 2H), 3.24 (t, J = 8.7 Hz, 2H), 2.05 (s, 3H), 1.97 (s, 3H). MS (ES⁺) m/z 382.3 (M + H)⁺; |
| | 2-((2,3-dihydrobenzofuran-7- | |
| | yl)amino)-N-(3-hydroxy-2,6- | |
| | dimethylphenyl)thiazole-5- | |
| | carboxamide. TFA salt | |
| BAA-531 | 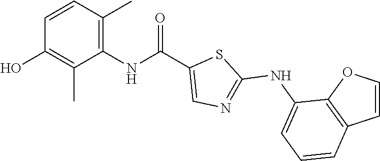 [SMILES: Cc1ccc(O)c(C)c1NC(=O)c1cnc(Nc2cccc3ccoc23)s1] | ¹H NMR (400 MHz, DMSO-d₆) δ 10.69 (s, 1H), 9.58 (s, 1H), 9.18 (s, 1H), 8.12- 8.03 (m, 3H), 7.37 (dd, J = 7.8, 1.1 Hz, 1H), 7.24 (t, J = 7.8 Hz, 1H), 7.01 (d, J = 2.1 Hz, 1H), 6.88 (d, J = 8.2 Hz, 1H), 6.68 (d, J = 8.2 Hz, 1H), 2.07 (s, 3H), 1.99 (s, 3H). MS (ES⁺) m/z 380.3 (M + H)⁺. |
| | 2-(benzofuran-7-ylamino)-N-(3- | |
| | hydroxy-2,6-dimethylphenyl)thiazole- | |
| | 5-carboxamide | |
| BAA-536 | 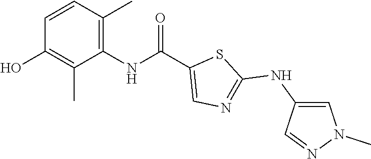 [SMILES: Cc1ccc(O)c(C)c1NC(=O)c1cnc(Nc2cnn(C)c2)s1] | ¹H NMR (400 MHz, DMSO-d₆) δ 10.25 (s, 1H), 9.47 (s, 1H), 9.16 (s, 1H), 8.01 (s, 1H), 7.93 (d, J = 0.8 Hz, 1H), 7.44 (d, J = 0.8 Hz, 1H), 6.87 (d, J = 8.2 Hz, 1H), 6.68 (d, J = 8.2 Hz, 1H), 3.82 (s, 3H), 2.05 (s, 3H), 1.97 (s, 3H). MS (ES⁺) m/z 344.3 (M + H)⁺; |
| | N-(3-hydroxy-2,6-dimethylphenyl)-2- | |
| | ((1-methyl-1H-pyrazol-4- | |
| | yl)amino)thiazole-5-carboxamide | |
| | hydrochloride | |
| BAA-537 | 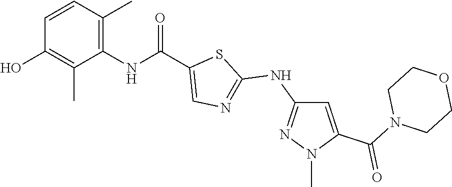 [SMILES: Cc1ccc(O)c(C)c1NC(=O)c1cnc(Nc2cc(C(=O)N3CCOCC3)n(C)n2)s1] | ¹H NMR (400 MHz, DMSO-d₆) δ 11.06 (br s, 1H), 9.52 (s, 1H), 9.20 (s, 1H), 8.10 (s, 1H), 6.88 (d, J = 8.2 Hz, 1H), 6.68 (d, J = 8.1 Hz, 1H), 6.17 (s, 1H), 3.79 (s, 3H), 3.68-3.49 (m, 8H), 2.06 (s, 3H), 1.98 (s, 3H). MS (ES⁺) m/z 457.3 (M + H)⁺. |
| | N-(3-hydroxy-2,6-dimethylphenyl)-2- | |
| | ((1-methyl-5-(morpholine-4-carbonyl)- | |
| | 1H-pyrazol-3-yl)amino)thiazole-5- | |
| | carboxamide | |
| BAA-539 | 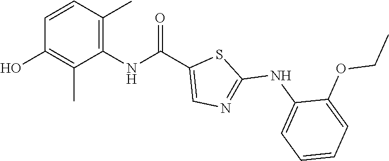 [SMILES: CCOc1ccccc1Nc1ncc(C(=O)Nc2c(C)ccc(O)c2C)s1] | ¹H NMR (400 MHz, DMSO-d₆) δ 9.74 (s, 1H), 9.53 (s, 1H), 9.17 (s, 1H), 8.18 (d, J = 7.6 Hz, 1H), 8.05 (s, 1H), 7.09-6.97 (m, 2H), 6.94 (ddd, J = 8.8, 6.5, 2.2 Hz, 1H), 6.88 (d, J = 8.2 Hz, 1H), 6.68 (d, J = 8.2 Hz, 1H), 4.13 (q, J = 7.0 Hz, 2H), 2.06 (s, 3H), 1.98 (s, 3H), 1.38 (t, J = 7.0 Hz, 3H). MS (ES⁺) m/z 384.3 (M + H)⁺; |
| | 2-((2-ethoxyphenyl)amino)-N-(3- | |
| | hydroxy-2,6-dimethylphenyl)thiazole- | |
| | 5-carboxamide. TFA salt | |
| BAA-540 | 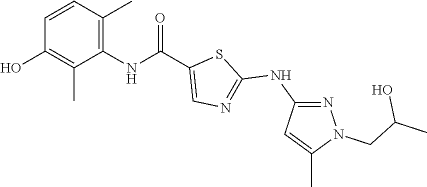 [SMILES: Cc1ccc(O)c(C)c1NC(=O)c1cnc(Nc2cc(C)n(CC(C)O)n2)s1] | ¹H NMR (400 MHz, DMSO-d₆) δ 11.07 (s, 1H), 9.51 (s, 1H), 9.16 (s, 1H), 8.11 (s, 1H), 6.88 (d, J = 8.2 Hz, 1H), 6.68 (d, J = 8.2 Hz, 1H), 5.77 (s, 1H), 4.02 (h, J = 5.9 Hz, 1H), 3.84 (qd, J = 13.8, 6.0 Hz, 2H), 2.24 (s, 3H), 2.06 (s, 3H), 1.98 (s, 3H), 1.08 (d, J = 6.3 Hz, 3H). MS (ES⁺) m/z 402.4 (M + H)⁺. |
| | N-(3-hydroxy-2,6-dimethylphenyl)-2- | |
| | ((1-(2-hydroxypropyl)-5-methyl-1H- | |
| | pyrazol-3-yl)amino)thiazole-5- | |
| | carboxamide | |
| BAA-543 | 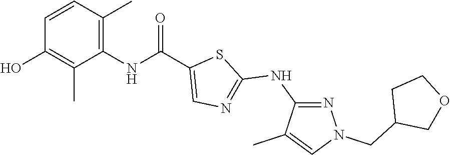 [SMILES: Cc1cn(CC2CCOC2)nc1Nc1ncc(C(=O)Nc2c(C)ccc(O)c2C)s1] | ¹H NMR (400 MHz, DMSO-d₆) δ 10.64 (s, 1H), 9.45 (s, 1H), 9.18 (s, 1H), 8.10 (s, 1H), 7.50 (d, J = 1.0 Hz, 1H), 6.87 (d, J = 8.2 Hz, 1H), 6.68 (d, J = 8.2 Hz, 1H), 4.05-3.90 (m, 2H), 3.79-3.56 (m, 3H), 3.50 (dd, J = 8.7, 5.5 Hz, 1H), 2.76- 2.63 (m, 1H), 2.06 (s, 3H), 2.01-1.96 (m, 6H), 1.96-1.86 (m, 1H), 1.60 (dq, J = 13.2, 6.9 Hz, 1H). MS (ES⁺) m/z 428.2 (M + H)⁺. |
| | N-(3-hydroxy-2,6-dimethylphenyl)-2- | |
| | ((4-methyl-1-(tetrahydrofuran-3- | |
| | yl)methyl)-1H-pyrazol-3- | |
| | yl)amino)thiazole-5-carboxamide | |
| BAA-544 | 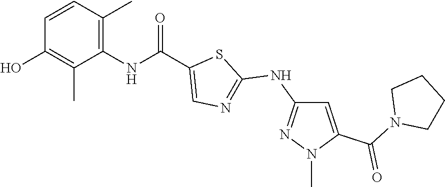 [SMILES: Cc1ccc(O)c(C)c1NC(=O)c1cnc(Nc2cc(C(=O)N3CCCC3)n(C)n2)s1] | ¹H NMR (400 MHz, DMSO-d₆) δ 11.03 (s, 1H), 9.52 (s, 1H), 9.18 (s, 1H), 8.09 (s, 1H), 6.88 (d, J = 8.2 Hz, 1H), 6.68 (d, J = 8.2 Hz, 1H), 6.31 (s, 1H), 3.86 (s, 3H), 3.53 (q, J = 5.3 Hz, 2H), 3.50-3.43 (m, 2H), 2.06 (s, 3H), 1.98 (s, 3H), 1.91- 1.83 (m, 4H). MS (ES⁺) m/z 441.2 (M + H)⁺. |
| | N-(3-hydroxy-2,6-dimethylphenyl)-2- | |
| | ((1-methyl-5-(pyrrolidine-1-carbonyl)- | |
| | 1H-pyrazol-3-yl)amino)thiazole-5- | |
| | carboxamide | |
| BAA-545 | 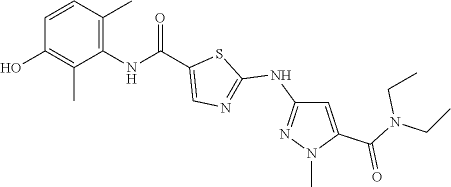 [SMILES: CCN(CC)C(=O)c1cc(Nc2ncc(C(=O)Nc3c(C)ccc(O)c3C)s2)nn1C] | ¹H NMR (400 MHz, DMSO-d₆) δ 10.87 (s, 1H), 9.37 (s, 1H), 8.97 (s, 1H), 8.04 (s, 1H), 6.84 (d, J = 8.1 Hz, 1H), 6.65 (d, J = 8.1 Hz, 1H), 6.14 (s, 1H), 3.70 (s, 3H), 3.37 (s, 4H), 2.04 (s, 3H), 1.97 (s, 3H), 1.10 (t, J = 7.0 Hz, 6H). MS (ES⁺) m/z 443.2 (M + H)⁺. |
| | 2-((5-(diethylcarbamoyl)-1-methyl-1H- | |
| | pyrazol-3-yl)amino)-N-(3-hydroxy-2,6- | |
| | dimethylphenyl)thiazole-5- | |
| | carboxamide | |
| BAA-548 | 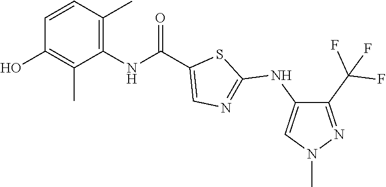 [SMILES: Cc1ccc(O)c(C)c1NC(=O)c1cnc(Nc2cn(C)nc2C(F)(F)F)s1] | ¹H NMR (400 MHz, DMSO-d₆) δ 9.97 (s, 1H), 9.50 (s, 1H), 9.17 (s, 1H), 8.34 (s, 1H), 8.00 (s, 1H), 6.87 (d, J = 8.2 Hz, 1H), 6.67 (d, J = 8.1 Hz, 1H), 3.92 (s, 3H), 2.05 (s, 3H), 1.97 (s, 3H). MS (ES⁺) m/z 412.2 (M + H)⁺. |
| | N-(3-hydroxy-2,6-dimethylphenyl)-2- | |
| | ((1-methyl-3-(trifluoromethyl)-1H- | |
| | pyrazol-4-yl)amino)thiazole-5- | |
| | carboxamide | |
| BAA-552 | 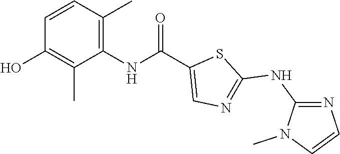 [SMILES: Cc1ccc(O)c(C)c1NC(=O)c1cnc(Nc2nccn2C)s1] | ¹H NMR (400 MHz, DMSO-d₆) δ 9.16 (s, 1H), 8.83 (s, 1H), 7.96 (s, 1H), 6.92- 6.84 (m, 2H), 6.81 (s, 1H), 6.68 (d, J = 8.2 Hz, 1H), 3.47 (s, 3H), 2.08 (s, 3H), 2.01 (s, 3H); MS (ES⁺) m/z 344.3 (M + H)⁺. |
| | N-(3-hydroxy-2,6-dimethylphenyl)-2- | |
| | ((1-methyl-1H-imidazol-2- | |
| | yl)amino)thiazole-5-carboxamide | |
| BAA-556 | 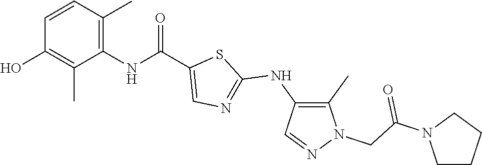 [SMILES: Cc1ccc(O)c(C)c1NC(=O)c1cnc(Nc2cnn(CC(=O)N3CCCC3)c2C)s1] | ¹H NMR (400 MHz, DMSO-d₆ + D₂O) δ 8.38 (s, 1H), 7.93 (s, 1H), 7.53 (s, 1H), 6.86 (d, J = 8.3 Hz, 1H), 6.65 (d, J = 8.2 Hz, 1H), 4.94 (s, 2H), 3.48 (t, J = 6.7 Hz, 2H), 3.29 (d, J = 13.7 Hz, 2H), 2.08 (s, 3H), 2.01 (s, 3H), 1.93 (s, 3H), 1.89 (q, J = 6.7 Hz, 2H), 1.78 (q, J = 6.7 Hz, 2H). MS (ES⁺) m/z 455.1 (M + H)⁺. |
| | N-(3-hydroxy-2,6-dimethylphenyl)-2- | |
| | ((5-methyl-1-(2-oxo-2-(pyrrolidin-1- | |
| | yl)ethyl)-1H-pyrazol-4- | |
| | yl)amino)thiazole-5-carboxamide | |
| BAA-558 | 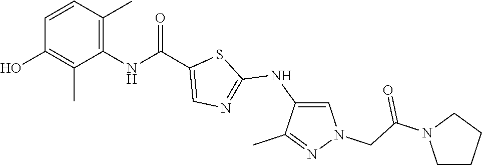 [SMILES: Cc1ccc(O)c(C)c1NC(=O)c1cnc(Nc2cn(CC(=O)N3CCCC3)nc2C)s1] | ¹H NMR (400 MHz, DMSO-d₆) δ 9.74 (s, 1H), 9.43 (s, 1H), 9.16 (s, 1H), 7.99 (s, 1H), 7.93 (s, 1H), 6.87 (d, J = 8.2 Hz, 1H), 6.67 (d, J = 8.2 Hz, 1H), 4.90 (s, 2H), 3.45 (t, J = 6.8 Hz, 2H), 3.29 (d, J = 6.9 Hz, 2H), 2.12 (s, 3H), 2.05 (s, 3H), 1.97 (s, 3H), 1.93-1.86 (m, 2H), 1.81- 1.73 (m, 2H). MS (ES⁺) m/z 453.2 (M − H)⁺. |
| | N-(3-hydroxy-2,6-dimethylphenyl)-2- | |
| | ((3-methyl-1-(2-oxo-2-(pyrrolidin-1- | |
| | yl)ethyl)-1H-pyrazol-4- | |
| | yl)amino)thiazole-5-carboxamide | |
| BAA-559 | 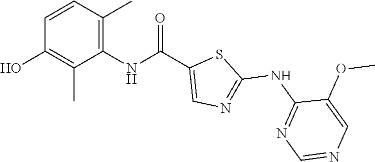 [SMILES: COc1cncnc1Nc1ncc(C(=O)Nc2c(C)ccc(O)c2C)s1] | ¹H NMR (400 MHz, DMSO-d₆) δ 11.41 (s, 1H), 9.68 (s, 1H), 9.19 (s, 1H), 8.56 (s, 1H), 8.30 (s, 1H), 8.28 (s, 1H), 6.89 (d, J = 8.2 Hz, 1H), 6.69 (d, J = 8.2 Hz, 1H), 3.96 (s, 3H), 2.08 (s, 3H), 1.99 (s, 3H). MS (ES⁺) m/z 372.1 (M + H)⁺. |
| | N-(3-hydroxy-2,6-dimethylphenyl)-2- | |
| | ((5-methoxypyrimidin-4-yl)amino) | |
| | thiazole-5-carboxamide | |
| BAA-562 | 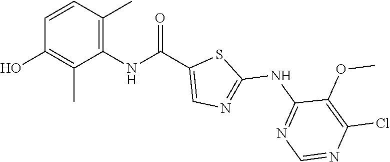 [SMILES: COc1c(Cl)ncnc1Nc1ncc(C(=O)Nc2c(C)ccc(O)c2C)s1] | ¹H NMR (400 MHz, DMSO-d₆) δ 12.24 (s, 1H), 9.70 (s, 1H), 9.19 (s, 1H), 8.54 (s, 1H), 8.31 (s, 1H), 6.89 (d, J = 8.2 Hz, 1H), 6.69 (d, J = 8.2 Hz, 1H), 3.88 (s, 3H), 2.07 (s, 3H), 1.99 (s, 3H). MS (ES⁺) m/z 406.2/408.1 (M + H)⁺. |
| | 2-((6-chloro-5-methoxypyrimidin-4- | |
| | yl)amino)-N-(3-hydroxy-2,6- | |
| | dimethylphenyl)thiazole-5- | |
| | carboxamide | |
| BAA-567 | 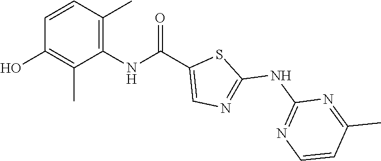 [SMILES: Cc1ccnc(Nc2ncc(C(=O)Nc3c(C)ccc(O)c3C)s2)n1] | ¹H NMR (400 MHz, DMSO-d₆) δ 11.96 (s, 1H), 9.60 (s, 1H), 9.17 (s, 1H), 8.53 (d, J = 5.0 Hz, 1H), 8.24 (s, 1H), 7.05- 6.95 (m, 1H), 6.89 (d, J = 8.2 Hz, 1H), 6.69 (d, J = 8.2 Hz, 1H), 2.47 (s, 3H), 2.07 (s, 3H), 1.99 (s, 3H). MS (ES⁺) m/z 356.3 (M + H)⁺. |
| | N-(3-hydroxy-2,6-dimethylphenyl)-2- | |
| | ((4-methylpyrimidin-2-yl)amino) | |
| | thiazole-5-carboxamide | |
| BAA-568 | 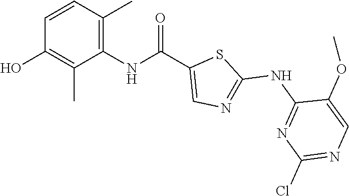 [SMILES: COc1cnc(Cl)nc1Nc1ncc(C(=O)Nc2c(C)ccc(O)c2C)s1] | ¹H NMR (400 MHz, DMSO-d₆) δ 9.69 (s, 1H), 9.20 (s, 1H), 8.30 (s, 1H), 8.12 (s, 1H), 6.89 (d, J = 8.2 Hz, 1H), 6.69 (d, J = 8.2 Hz, 1H), 3.92 (s, 3H), 2.08 (s, 3H), 2.00 (s, 3H). MS (ES⁺) m/z 406.1/408.1 (M + H)⁺. |
| | 2-((2-chloro-5-methoxypyrimidin-4- | |
| | yl)amino)-N-(3-hydroxy-2,6- | |
| | dimethylphenyl)thiazole-5- | |
| | carboxamide | |
| BAA-570 | 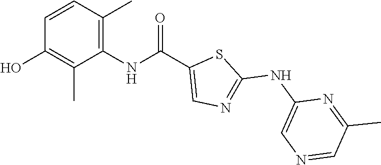 [SMILES: Cc1cncc(Nc2ncc(C(=O)Nc3c(C)ccc(O)c3C)s2)n1] | ¹H NMR (400 MHz, DMSO-d₆) δ 11.98 (s, 1H), 9.62 (s, 1H), 9.19 (s, 1H), 8.37- 8.22 (m, 2H), 8.09 (s, 1H), 6.89 (d, J = 8.2 Hz, 1H), 6.69 (d, J = 8.2 Hz, 1H), 2.08 (s, 3H), 2.00 (s, 3H). MS (ES⁺) m/z 356.1 (M + H)⁺. |
| | N-(3-hydroxy-2,6-dimethylphenyl)-2- | |
| | ((6-methylpyrazin-2-yl)amino)thiazole- | |
| | 5-carboxamide | |
| BAA-571 | 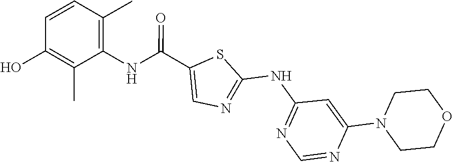 [SMILES: Cc1ccc(O)c(C)c1NC(=O)c1cnc(Nc2cc(N3CCOCC3)ncn2)s1] | ¹H NMR (400 MHz, DMSO-d₆) δ 11.55 (s, 1H), 9.55 (s, 1H), 9.17 (s, 1H), 8.41 (d, J = 0.9 Hz, 1H), 8.17 (s, 1H), 6.88 (d, J = 8.2 Hz, 1H), 6.68 (d, J = 8.2 Hz, 1H), 6.20 (s, 1H), 3.74-3.64 (m, 4H), 3.55- 3.45 (m, 4H), 2.06 (s, 3H), 1.98 (s, 3H). MS (ES⁺) m/z 427.4 (M + H)⁺. |
| | N-(3-hydroxy-2,6-dimethylphenyl)-2- | |
| | ((6-morpholinopyrimidin-4-yl)amino) | |
| | thiazole-5-carboxamide | |
| BAA-572 | 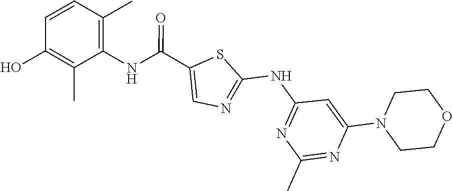 [SMILES: Cc1nc(Nc2ncc(C(=O)Nc3c(C)ccc(O)c3C)s2)cc(N2CCOCC2)n1] | ¹H NMR (400 MHz, DMSO-d₆) δ 11.44 (s, 1H), 9.56 (s, 1H), 9.18 (s, 1H), 8.18 (s, 1H), 6.89 (d, J = 8.2 Hz, 1H), 6.69 (d, J = 8.2 Hz, 1H), 6.04 (s, 1H), 3.72-3.61 (m, 4H), 3.52-3.45 (m, 4H), 2.41 (s, 3H), 2.07 (s, 3H), 1.99 (s, 3H). MS (ES⁺) m/z 441.3 (M + H)⁺. |
| | N-(3-hydroxy-2,6-dimethylphenyl)-2- | |
| | ((2-methyl-6-morpholinopyrimidin-4- | |
| | yl)amino)thiazole-5-carboxamide | |
| BAA-580 | 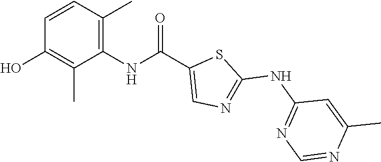 [SMILES: Cc1cc(Nc2ncc(C(=O)Nc3c(C)ccc(O)c3C)s2)ncn1] | ¹H NMR (400 MHz, MeOD) δ 8.74 (d, J = 1.1 Hz, 1H), 8.16 (s, 1H), 6.94-6.86 (m, 2H), 6.67 (d, J = 8.2 Hz, 1H), 2.44 (d, J = 0.7 Hz, 3H), 2.14 (d, J = 0.7 Hz, 3H), 2.09 (s, 3H). MS (ES⁺) m/z 356.1 (M + H)⁺. |
| | N-(3-hydroxy-2,6-dimethylphenyl)-2- | |
| | ((6-methylpyrimidin-4-yl)amino) | |
| | thiazole-5-carboxamide | |
| BAA-581 | 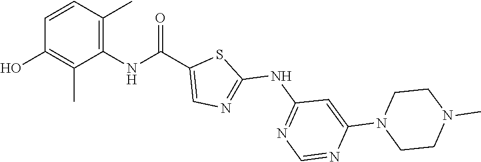 [SMILES: Cc1ccc(O)c(C)c1NC(=O)c1cnc(Nc2cc(N3CCN(C)CC3)ncn2)s1] | ¹H NMR (400 MHz, MeOD) δ 8.36 (d, J = 0.9 Hz, 1H), 8.09 (s, 1H), 6.89 (d, J = 8.2 Hz, 1H), 6.66 (d, J = 8.3 Hz, 1H), 6.19 (s, 1H), 3.64 (t, J = 5.2 Hz, 4H), 2.63 (s, 24H), 2.51 (t, J = 5.2 Hz, 4H), 2.33 (s, 3H), 2.14 (s, 3H), 2.09 (s, 3H). MS (ES⁺) m/z 440.3 (M + H)⁺. |
| | N-(3-hydroxy-2,6-dimethylphenyl)-2- | |
| | ((6-(4-methylpiperazin-1-yl)pyrimidin- | |
| | 4-yl)amino)thiazole-5-carboxamide | |
| BAA-582 | 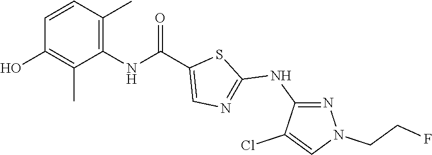 [SMILES: Cc1ccc(O)c(C)c1NC(=O)c1cnc(Nc2nn(CCF)cc2Cl)s1] | ¹H NMR (400 MHz, DMSO-d₆) δ 9.51 (s, 1H), 9.17 (s, 1H), 8.09 (s, 1H), 8.02 (s, 1H), 6.88 (d, J = 8.2 Hz, 1H), 6.68 (d, J = 8.1 Hz, 1H), 4.84 (t, J = 4.6 Hz, 1H), 4.72 (t, J = 4.6 Hz, 1H), 4.41 (t, J = 4.6 Hz, 1H), 4.34 (t, J = 4.6 Hz, 1H), 2.06 (s, 3H), 1.98 (s, 3H). MS (ES⁺) m/z 410.2/412.1 (M + H)⁺. |
| | 2-((4-chloro-1-(2-fluoroethyl)-1H- | |
| | pyrazol-3-yl)amino)-N-(3-hydroxy-2,6- | |
| | dimethylphenyl)thiazole-5- | |
| | carboxamide | |
| BAA-585 | 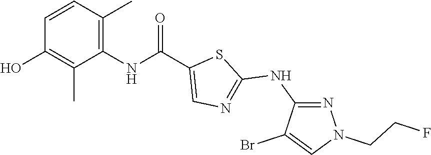 [SMILES: Cc1ccc(O)c(C)c1NC(=O)c1cnc(Nc2nn(CCF)cc2Br)s1] | ¹H NMR (400 MHz, DMSO-d₆) δ 9.50 (s, 1H), 9.18 (s, 1H), 8.08 (s, 1H), 8.00 (s, 1H), 6.88 (d, J = 8.2 Hz, 1H), 6.68 (d, J = 8.2 Hz, 1H), 4.78 (dt, J = 47.2, 4.6 Hz, 2H), 4.39 (dt, J = 27.7, 4.7 Hz, 2H), 2.06 (s, 3H), 1.98 (s, 3H). MS (ES⁺) m/z 454.1/456.1 (M + H)⁺. |
| | 2-((4-bromo-1-(2-fluoroethyl)-1H- | |
| | pyrazol-3-yl)amino)-N-(3-hydroxy-2,6- | |
| | dimethylphenyl)thiazole-5- | |
| | carboxamide | |
| BAA-586 | 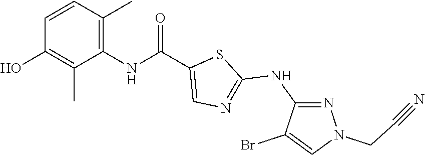 [SMILES: Cc1ccc(O)c(C)c1NC(=O)c1cnc(Nc2nn(CC#N)cc2Br)s1] | ¹H NMR (400 MHz, DMSO-d₆) δ 9.53 (s, 1H), 9.18 (s, 1H), 8.10 (s, 1H), 8.04 (s, 1H), 6.88 (d, J = 8.2 Hz, 1H), 6.68 (d, J = 8.1 Hz, 1H), 5.43 (s, 2H), 2.06 (s, 3H), 1.98 (s, 3H). MS (ES⁺) m/z 447.1/449.1 (M + H)⁺. |
| | 2-((4-bromo-1-(cyanomethyl)-1H- | |
| | pyrazol-3-yl)amino)-N-(3-hydroxy-2,6- | |
| | dimethylphenyl)thiazole-5- | |
| | carboxamide | |
| BAA-587 | 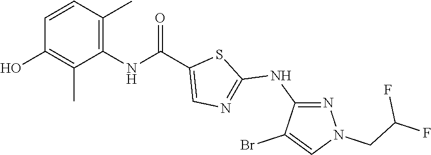 [SMILES: Cc1ccc(O)c(C)c1NC(=O)c1cnc(Nc2nn(CC(F)F)cc2Br)s1] | ¹H NMR (400 MHz, DMSO-d₆) δ 9.44 (s, 1H), 9.17 (s, 1H), 8.07 (s, 1H), 7.98 (s, 1H), 6.87 (d, J = 8.2 Hz, 1H), 6.68 (d, J = 8.2 Hz, 1H), 6.36 (t, J = 54.9 Hz, 1H), 4.57 (t, J = 13.2 Hz, 2H), 2.06 (s, 3H), 1.98 (s, 3H). MS (ES⁺) m/z 472.0/474.0 (M + H)⁺. |
| | 2-((4-bromo-1-(2,2-difluoroethyl)-1H- | |
| | pyrazol-3-yl)amino)-N-(3-hydroxy-2,6- | |
| | dimethylphenyl)thiazole-5- | |
| | carboxamide | |
| BAA-588 | 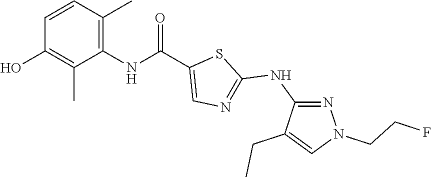 [SMILES: CCc1cn(CCF)nc1Nc1ncc(C(=O)Nc2c(C)ccc(O)c2C)s1] | ¹H NMR (400 MHz, DMSO-d₆) δ 10.70 (s, 1H), 9.46 (s, 1H), 9.16 (s, 1H), 8.10 (s, 1H), 7.53 (s, 1H), 6.87 (d, J = 8.2 Hz, 1H), 6.67 (d, J = 8.2 Hz, 1H), 4.76 (dt, J = 47.3, 4.8 Hz, 2H), 4.31 (dt, J = 27.8, 4.8 Hz, 2H), 2.45 (q, J = 7.6 Hz, 2H), 2.06 (s, 3H), 1.98 (s, 3H), 1.11 (t, J = 7.4 Hz, 3H). MS (ES⁺) m/z 404.4 (M + H)⁺. |
| | 2-((4-ethyl-1-(2-fluoroethyl)-1H- | |
| | pyrazol-3-yl)amino)-N-(3-hydroxy-2,6- | |
| | dimethylphenyl)thiazole-5- | |
| | carboxamide | |
| BAA-589 | 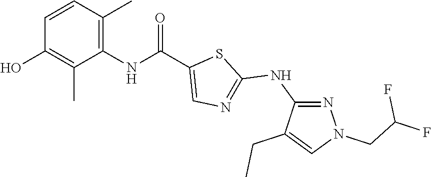 [SMILES: CCc1cn(CC(F)F)nc1Nc1ncc(C(=O)Nc2c(C)ccc(O)c2C)s1] | ¹H NMR (400 MHz, DMSO-d₆) δ 10.76 (s, 1H), 9.47 (s, 1H), 9.17 (s, 1H), 8.10 (s, 1H), 7.55 (s, 1H), 6.88 (d, J = 8.2 Hz, 1H), 6.68 (d, J = 8.1 Hz, 1H), 6.32 (tt, J = 54.9, 3.8 Hz, 1H), 4.50 (td, J = 15.1, 4.0 Hz, 2H), 2.45 (q, J = 7.3 Hz, 2H), 2.06 (s, 3H), 1.98 (s, 3H), 1.11 (t, J = 7.5 Hz, 3H). MS (ES⁺) m/z 422.2 (M + H)⁺. |
| | 2-((1-(2,2-difluoroethyl)-4-ethyl-1H- | |
| | pyrazol-3-yl)amino)-N-(3-hydroxy-2,6- | |
| | dimethylphenyl)thiazole-5- | |
| | carboxamide | |
| BAA-590 | 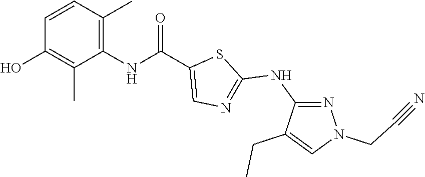 [SMILES: CCc1cn(CC#N)nc1Nc1ncc(C(=O)Nc2c(C)ccc(O)c2C)s1] | ¹H NMR (400 MHz, DMSO-d₆) δ 10.88 (s, 1H), 9.51 (s, 1H), 9.17 (s, 1H), 8.13 (s, 1H), 7.60 (s, 1H), 6.88 (d, J = 8.6 Hz, 1H), 6.68 (d, J = 8.2 Hz, 1H), 5.37 (s, 2H), 2.46 (q, J = 7.4 Hz, 2H), 2.07 (s, 3H), 1.99 (s, 3H), 1.11 (t, J = 7.5 Hz, 3H). MS (ES⁺) m/z 397.2 (M + H)⁺. |
| | 2-((1-(cyanomethyl)-4-ethyl-1H- | |
| | pyrazol-3-yl)amino)-N-(3-hydroxy-2,6- | |
| | dimethylphenyl)thiazole-5- | |
| | carboxamide. TFA salt | |
| BAA-601 | 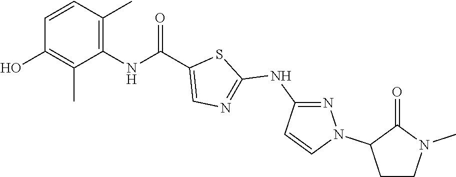 [SMILES: Cc1ccc(O)c(C)c1NC(=O)c1cnc(Nc2ccn(C3CCN(C)C3=O)n2)s1] | ¹H NMR (400 MHz, MeOD) δ 8.02 (s, 1H), 7.61 (d, J = 2.4 Hz, 1H), 6.92-6.85 (m, 1H), 6.66 (d, J = 8.2 Hz, 1H), 5.98 (d, J = 2.4 Hz, 1H), 4.97 (t, J = 7.7 Hz, 1H), 3.84-3.74 (m, 1H), 3.56-3.46 (m, 1H), 2.93 (d, J = 0.6 Hz, 3H), 2.62-2.51 (m, 2H), 2.16-2.11 (m, 3H), 2.09 (s, 3H). MS (ES⁺) m/z 427.4 (M + H)⁺. |
| | N-(3-hydroxy-2,6-dimethylphenyl)-2- | |
| | ((1-(1-methyl-2-oxopyrrolidin-3-yl)-1H- | |
| | pyrazol-3-yl)amino)thiazole-5- | |
| | carboxamide | |
| | 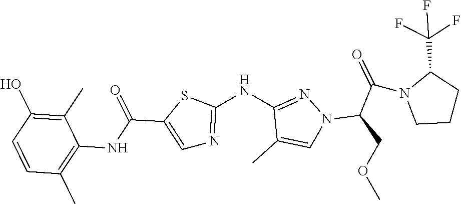 [SMILES: COC[C@H](C(=O)N1CCC[C@H]1C(F)(F)F)n1cc(C)c(Nc2ncc(C(=O)Nc3c(C)ccc(O)c3C)s2)n1] | ¹H NMR (400 MHz, DMSO-d₆) δ 10.78 (s, 1H), 9.48 (s, 1H), 9.17 (s, 1H), 8.11 (s, 1H), 7.57 (d, J = 1.0 Hz, 1H), 6.88 (d, J = 8.2 Hz, 1H), 6.68 (d, J = 8.2 Hz, 1H), 5.38 (dd, J = 8.1, 5.3 Hz, 1H), 4.77 (p, J = 8.0 Hz, 1H), 3.94 (dd, J = 10.4, 8.1 Hz, 1H), 3.86-3.71 (m, 3H), 3.30 (s, 3H), 2.16-1.89 (m, 13H). MS (ES⁺) m/z 567.3 (M + H)⁺. |
| | N-(3-hydroxy-2,6-dimethylphenyl)-2- | |
| | ((1-((R)-3-methoxy-1-oxo-1-((S)-2- | |
| | (trifluoromethyl)pyrrolidin-1-yl)propan- | |
| | 2-yl)-4-methyl-1H-pyrazol-3-yl)amino) | |
| | thiazole-5-carboxamide | |
| | 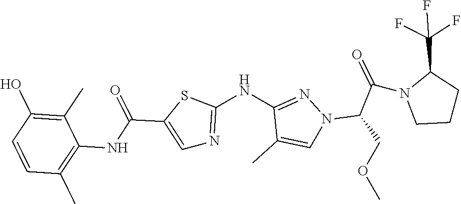 [SMILES: COC[C@@H](C(=O)N1CCC[C@@H]1C(F)(F)F)n1cc(C)c(Nc2ncc(C(=O)Nc3c(C)ccc(O)c3C)s2)n1] | ¹H NMR (400 MHz, DMSO-d₆) δ 10.77 (s, 1H), 9.48 (s, 1H), 9.17 (s, 1H), 8.11 (s, 1H), 7.57 (d, J = 1.0 Hz, 1H), 6.88 (d, J = 8.2 Hz, 1H), 6.68 (d, J = 8.2 Hz, 1H), 5.38 (dd, J = 8.1, 5.3 Hz, 1H), 4.77 (p, J = 8.0 Hz, 1H), 3.94 (dd, J = 10.4, 8.1 Hz, 1H), 3.88-3.68 (m, 3H), 3.30 (s, 3H), 2.20-1.83 (m, 13H). MS (ES⁺) m/z 567.2 (M + H)⁺. |
| | N-(3-hydroxy-2,6-dimethylphenyl)-2- | |
| | ((1-((S)-3-methoxy-1-oxo-1-((R)-2- | |
| | (trifluoromethyl)pyrrolidin-1-yl)propan- | |
| | 2-yl)-4-methyl-1H-pyrazol-3- | |
| | yl)amino)thiazole-5-carboxamide | |

Buchwald Coupling of Aminothiazoles with Aryl Halides
General Method N: Buchwald Coupling of Aminothiazoles with Substituted Aryl Halides

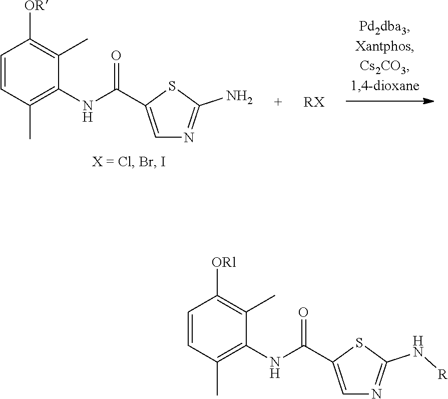

A mixture of aminothiazole (1.0 eq), substituted amine (1.0-1.5 eq), cesium carbonate (1.1-2.0 eq), Pd₂dba₃ (10%), Xantphos (20%) and 1,4-dioxane (0.1-0.3 M) was degassed with nitrogen and stirred at reflux for 6-24 h. After cooling to rt, the mixture was diluted H₂O and extracted with EOAc (3×). The combined organic phases were dried over MgSO₄, filtered and the solvent was removed under reduced pressure. The crude may be purified by chromatography or other methods if required.

### Representative Example

### N-(3-Hydroxy-2,6-dimethylphenyl)-2-((3-methyl-6-(4-(trifluoromethyl)benzamido)pyridin-2-yl)amino)thiazole-5-carboxamide( ) BAA-328

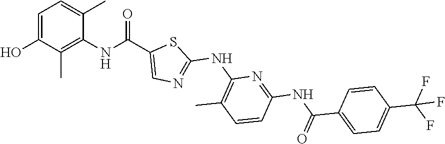 [SMILES: Cc1ccc(NC(=O)c2ccc(C(F)(F)F)cc2)nc1Nc1ncc(C(=O)Nc2c(C)ccc(O)c2C)s1]

A mixture of N-(6-bromo-5-methyl-2-pyridyl)-4-(trifluoromethyl)benzamide (120 mg, 0.334 mmol, 1.10 eq), 2-amino-N-(3-hydroxy-2,6-dimethyl-phenyl)thiazole-5-carboxamide (80 mg, 0.304 mmol, 1.00 eq), tris(dibenzylideneacetone) dipalladium (0)(28 mg, 0.0304 mmol, 0.100 eq), 4,5-bis(diphenylphospheno)-9,9-dimethylxanthene (35 mg, 0.0608 mmol, 0.200 eq), cesium carbonate
(110 mg, 0.334 mmol, 1.10 eq) and 1,4-dioxane (2.5 mL) was degassed with nitrogen and stirred at 95° C. for 16 h. After cooling to rt, the mixture was diluted with H₂O (10 mL) and extracted with EtOAc (3×10 mL). The combined organic phases was dried over MgSO₄ and filtered. The solvent was removed under reduced pressure. The crude was purified by RP chromatography (MeCN/H₂O 5>95%) and then triturated with DCM to afford N-(3-hydroxy-2,6-dimethyl-phenyl)-2-[3-methyl-6-[[4-(trifluoromethyl)benzoyl]amino]-2-pyridyl]amino]thiazole-5-carboxamide (26 mg, 0.0480 mmol, 16%) as white solids. ¹H NMR (300 MHz, Methanol-d4/chloroform -d) δ 8.23-8.02 (m, 3H), 7.90-7.70 (m, 3H), 7.68-7.50 (m, 1H), 6.89 (d, J=8.2 Hz, 1H), 6.68 (d, J=8.3 Hz, 1H), 2.36 (s, 3H), 2.16 (s, 3H), 2.12 (s, 3H). ¹⁹F NMR (282 MHz, Methanol-d4/chloroform -d) δ-63.79. MS (ES-) m/z 540.3 (M−H)⁻.

The following example compounds were prepared similarly using Method N using the appropriately substituted halides.

| # | Structure and Name | Substrate | Analytical data |
|---|---|---|---|
| BAA- 329 | 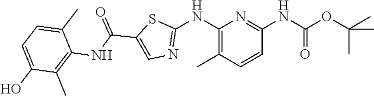 [SMILES: Cc1ccc(NC(=O)OC(C)(C)C)nc1Nc1ncc(C(=O)Nc2c(C)ccc(O)c2C)s1] | 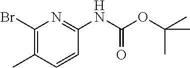 | ¹H NMR (300 MHz, Methanol- d4/chloroform-d) δ 8.14 (s, 1H), 7.50-7.39 (m, 2H), 6.89 (d, J = 8.3 Hz, 1H), 6.68 (d, J = 8.2 Hz, 1H), 2.28 (s, 3H), 2.17 (s, 3H), 2.13 (s, 3H), 1.52 (s, 9H). MS (ES+) m/z 470.2 (M + H)⁺ |
| | tert-butyl (6-((5-((3-hydroxy- | | |
| | 2,6-dimethylphenyl) | | |
| | carbamoyl)thiazol-2- | | |
| | yl)amino)-5-methylpyridin-2- | | |
| | yl)carbamate | | |

General Method Q: Buchwald Coupling of Aminothiazoles with Aryl Halides and Subsequent HCl Salt Formation

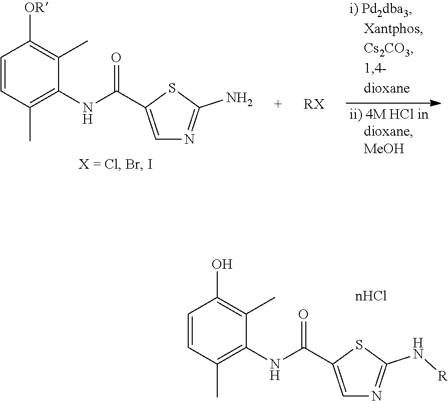

A mixture of bromothiazole (1.0 eq), substituted amine (1.0-1.5 eq), cesium carbonate (1.1-2.0 eq), Pd₂dba₃ (10%), Xantphos (20%) and 1,4-dioxane (0.1-0.3 M) was degassed with nitrogen and stirred at reflux for 6-24 h. After cooling to rt, the mixture was diluted H₂O and extracted with EOAc (3×). The combined organic phases were dried over MgSO₄, filtered and the solvent was removed under reduced pressure. The crude may be purified by chromatography or other methods if required to afford the product as the free base. This was dissolved in MeOH(0.1-0.3 M) and treated with an equal volume of 4 M HCl in dioxane for 1 min before the solvent was removed under reduced pressure to afford the desired hydrochloride.

### Representative Example

N-(3-Hydroxy-2.6-dimethylphenyl)-2-((3-methoxy-6-pivalamidopyridin-2-yl)amino)thiazole-5-carboxamide hydrochloride( ) BAA-330

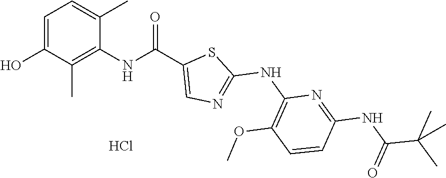 [SMILES: COc1ccc(NC(=O)C(C)(C)C)nc1Nc1ncc(C(=O)Nc2c(C)ccc(O)c2C)s1 ; Cl]

A mixture of N-(6-bromo-5-methoxy-2-pyridyl)-2,2-dimethyl-propanamide (87 mg, 0.304 mmol, 1.00 eq), 2-amino-N-(3-hydroxy-2,6-dimethyl-phenyl)thiazole-5-carboxamide (80 mg, 0.304 mmol, 1.00 eq), tris(dibenzylideneacetone) dipalladium (0)(28 mg, 0.0304 mmol, 0.100 eq), 4,5-bis(diphenylphospheno)-9,9-dimethylxanthene (35 mg, 0.0608 mmol, 0.200 eq), cesium carbonate
(129 mg, 0.395 mmol, 1.30 eq) and 1,4-dioxane (2.5 mL) was degassed with nitrogen and stirred at 95° C. for 16 h. After cooling to rt, the mixture was diluted with H₂O (10 mL) and extracted with EtOAc (3×10 mL). The combined organic phases was dried over MgSO₄ and filtered. The solvent was removed under reduced pressure. The crude was purified by RP chromatography (MeCN/H2O 5>95%) and triturated with DCM to afford the desired compound as the free base. This was dissolved in MeOH(1.0 mL) and treated with 4 M HCl in dioxane (1.0 mL) for 1 min before the solvent was removed under reduced pressure to afford N-(3-hydroxy-2,6-dimethylphenyl)-2-((3-methoxy-6-pivalamidopyridin-2-yl)amino)thiazole-5-carboxamide hydrochloride (63 mg, 0.125 mmol, 41%) as beige solids. ¹H NMR (300 MHz, Methanol-d4/chloroform -d) δ 8.51 (s, 1H), 7.64-7.53 (m, 2H), 6.88 (d, J=8.3 Hz, 1H), 6.69 (d, J=8.2 Hz, 1H), 4.00 (s, 3H), 2.15 (s, 3H), 2.10 (s, 3H), 1.35 (s, 9H). MS (ES+) m/z 470.2 (M+H)⁺.

The following example compounds were prepared similarly using Method Q using the appropriately substituted amine.

| # | Structure and Name | Substrate | Analytical data |
|---|---|---|---|
| BAA-331 | 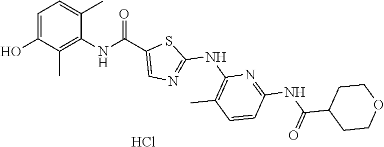 [SMILES: Cc1ccc(NC(=O)C2CCOCC2)nc1Nc1ncc(C(=O)Nc2c(C)ccc(O)c2C)s1 ; Cl] | 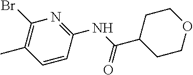 | ¹H NMR (300 MHz, Methanol- d4/chloroform-d) δ 8.44 (s, 1H), 7.73 (d, J = 8.2 Hz, 1H), 7.22 (br, 1H), 6.93 (d, J = 8.1 Hz, 1H), 6.72 (d, J = 8.2 Hz, 1H), 4.08-3.98 (m, 2H), 3.58-3.44 (m, 2H), 2.86-2.71 (m, 1H), 2.39 (s, 3H), 2.17 (s, 3H), 2.12 (s, 3H), 2.00-1.78 (m, 4H). MS (ES+) m/z 482.2 (M + H)⁺ |
| | N-(3-hydroxy-2,6- | | |
| | dimethylphenyl)-2-((3-methyl-6- | | |
| | (tetrahydro-2H-pyran-4- | | |
| | carboxamido)pyridin-2- | | |
| | yl)amino)thiazole-5-carboxamide | | |
| | hydrochloride | | |
| BAA-332 | 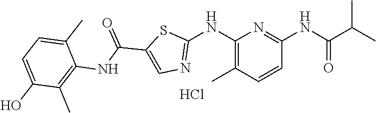 [SMILES: Cc1ccc(NC(=O)C(C)C)nc1Nc1ncc(C(=O)Nc2c(C)ccc(O)c2C)s1 ; Cl] N-(3-hydroxy-2,6-dimethylphenyl)-2-((6- isobutyramido-3-methylpyridin-2- yl)amino)thiazole-5-carboxamide hydrochloride | 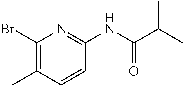 | ¹H NMR (300 MHz, Methanol- d4/chloroform-d) δ 8.50 (s, 1H), 7.75 (d, J = 8.3 Hz, 1H), 7.36 (br, 1H), 6.91 (d, J = 8.2 Hz, 1H), 6.71 (d, J = 8.2 Hz, 1H), 2.84 (h, J = 6.8 Hz, 1H), 2.42 (s, 3H), 2.17 (s, 3H), 2.13 (s, 3H), 1.26 (d, J = 6.8 Hz, 6H). MS (ES+) m/z 440.2 (M + H)⁺ |
| BAA-333 | 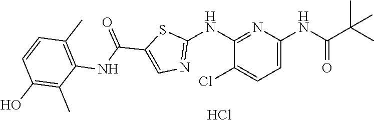 [SMILES: Cc1ccc(O)c(C)c1NC(=O)c1cnc(Nc2nc(NC(=O)C(C)(C)C)ccc2Cl)s1 ; Cl] | 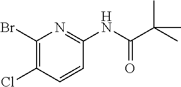 | ¹H NMR (300 MHz, Methanol- d4/chloroform-d) δ 8.34 (s, 1H), 7.78 (d, J = 8.6 Hz, 1H), 7.65 (d, J = 8.8 Hz, 1H), 6.86 (d, J = 8.2 Hz, 1H), 6.68 (d, J = 8.2 Hz, 1H), 2.15 (s, 3H), 2.10 (s, 3H), 1.32 (s, 9H). MS (ES+) m/z 474.1 (M + H)⁺ |
| | 2-((3-chloro-6-pivalamidopyridin- | | |
| | 2-yl)amino)-N-(3-hydroxy-2,6- | | |
| | dimethylphenyl)thiazole-5- | | |
| | carboxamide hydrochloride | | |
| BAA-334 | 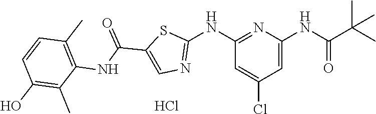 [SMILES: Cc1ccc(O)c(C)c1NC(=O)c1cnc(Nc2cc(Cl)cc(NC(=O)C(C)(C)C)n2)s1 ; Cl] | 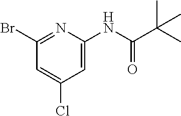 | ¹H NMR (300 MHz, Methanol- d4/chloroform-d) δ 8.52 (s, 1H), 7.82 (d, J = 1.4 Hz, 1H), 6.94 (d, J = 1.5 Hz, 1H), 6.89 (d, J = 8.3 Hz, 1H), 6.70 (d, J = 8.2 Hz, 1H), 2.17 (s, 3H), 2.12 (s, 3H), 1.38 (s, 9H). MS (ES+) m/z 474.1 (M + H)⁺ |
| | 2-((4-chloro-6-pivalamidopyridin- | | |
| | 2-yl)amino)-N-(3-hydroxy-2,6- | | |
| | dimethylphenyl)thiazole-5- | | |
| | carboxamide hydrochloride | | |
| BAA-335 | 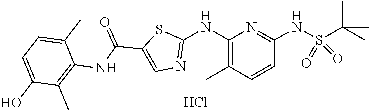 [SMILES: Cc1ccc(NS(=O)(=O)C(C)(C)C)nc1Nc1ncc(C(=O)Nc2c(C)ccc(O)c2C)s1 ; Cl] 2-((6-((1,1- dimethylethyl)sulfonamido)-3- methylpyridin-2-yl)amino)-N-(3- hydroxy-2,6-dimethylphenyl)thiazole-5- carboxamide hydrochloride | 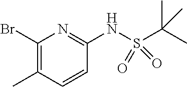 | ¹H NMR (300 MHz, Methanol- d4/chloroform-d) δ 8.45 (s, 1H), 7.67 (dd, J = 8.2, 0.8 Hz, 1H), 6.99 (d, J = 8.0 Hz, 1H), 6.90 (d, J = 8.3 Hz, 1H), 6.70 (d, J = 8.2 Hz, 1H), 2.39 (s, 3H), 2.15 (s, 3H), 2.11 (s, 3H), 1.42 (s, 9H). MS (ES+) m/z 490.2 (M + H)⁺ |
| BAA-336 | 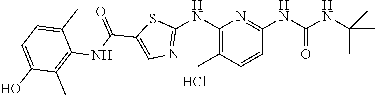 [SMILES: Cc1ccc(NC(=O)NC(C)(C)C)nc1Nc1ncc(C(=O)Nc2c(C)ccc(O)c2C)s1 ; Cl] 2-((6-(3-(tert-butyl)ureido)-3- methylpyridin-2-yl)amino)-N-(3- hydroxy-2,6-dimethylphenyl)thiazole-5- carboxamide hydrochloride | 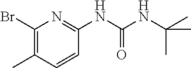 | ¹H NMR (300 MHz, Methanol- d4/chloroform-d) δ 8.41 (s, 1H), 7.72 (d, J = 8.4 Hz 1H), 7.16-6.92 (m, 1H), 6.88 (d, J = 8.3 Hz, 1H), 6.68 (d, J = 8.1 Hz, 1H), 2.38 (s, 3H), 2.16 (s, 3H), 2.11 (s, 3H), 1.40 (s, 9H). MS (ES+) m/z 469.2 (M + H)⁺ |
| BAA-234 | 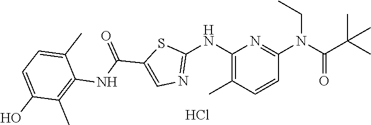 [SMILES: CCN(C(=O)C(C)(C)C)c1ccc(C)c(Nc2ncc(C(=O)Nc3c(C)ccc(O)c3C)s2)n1 ; Cl] 2-((6-(N-ethylpivalamido)-3- methylpyridin-2-yl)amino)-N-(3- hydroxy-2,6-dimethylphenyl) thiazole-5-carboxamide | 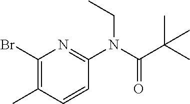 | ¹H NMR (300 MHz, MeOD) δ 8.16 (s, 1H), 7.62 (dd, J = 7.6, 0.9 Hz, 1H), 6.89 (d, J = 8.2 Hz, 1H), 6.83 (d, J = 7.5 Hz, 1H), 6.68 (d, J = 8.2 Hz, 1H), 3.79 (q, J = 7.1 Hz, 2H), 2.42 (s, 3H), 2.16 (s, 3H), 2.12 (s, 3H), 1.15 (t, J = 7.1 Hz, 3H), 1.03 (s, 9H). MS (ES+) m/z 482.2 (M + H)+ |

### 2-((2-((3,3-Dimethylbutyl)amino)-6-methylpyrimidin-4-yl)amino)-N-(3-hydroxy-2,6-dimethylphenyl)thiazole-5-carboxamide( ) BAA-338

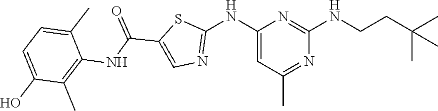 [SMILES: Cc1cc(Nc2ncc(C(=O)Nc3c(C)ccc(O)c3C)s2)nc(NCCC(C)(C)C)n1]

A mixture 2-[(2-chloro-6-methyl-pyrimidin-4-yl)amino]-N-(3-hydroxy-2,6-dimethyl-phenyl)thiazole-5-carboxamide (50 mg, 0.128 mmol, 1.00 eq), 3,3-dimethylbutylamine (0.052 mL, 0.385 mmol, 3.00 eq) and tert-butanol (1.4 mL) was stirred at 90° C. for 24 h. After cooling to rt, the mixture was diluted with H₂O (10 mL) and extracted with EtOAc (3×10 mL). The combined organic phases were dried over MgSO₄, filtered and the solvent was removed under reduced pressure. The crude was purified by RP chromatography (MeCN/H2O 5>95%) to afford 2-[2-(3,3-dimethylbutylamino)-6-methyl-pyrimidin-4-yl]amino]-N-(3-hydroxy-2,6-dimethyl-phenyl)thiazole-5-carboxamide (20 mg, 0.0440 mmol, 34%) as white solids. ¹H NMR (300 MHz,) δ 11.47 (br, 1H), 9.55 (s, 1H), 9.20 (s, 1H), 8.20 (s, 1H), 6.99 (br, 1H), 6.88 (d, J=8.2 Hz, 1H), 6.68 (d, J=8.2 Hz, 1H), 6.04 (s, 1H), 3.50-3.24 (m, 2H), 2.15 (s, 3H), 2.06 (s, 3H), 1.98 (s, 3H), 1.53-1.41 (m, 2H), 0.91 (s, 9H). MS (ES+) m/z 455.2 (M+H)⁺.

### N-(3-Hydroxy-2.6-dimethylphenyl)-2-((6-pivalamidopyridin-2-yl)amino)thiazole-5-carboxamide ( ) BAA-339

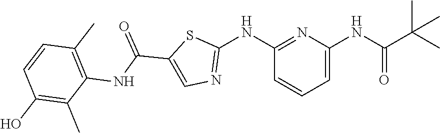 [SMILES: Cc1ccc(O)c(C)c1NC(=O)c1cnc(Nc2cccc(NC(=O)C(C)(C)C)n2)s1]

A mixture of 2-[(6-amino-2-pyridyl)amino]-N-(3-hydroxy-2,6-dimethyl-phenyl)thiazole-5-carboxamide (99 mg, 0.279 mmol, 1.00 eq), trimethylacetyl chloride (0.038 mL, 0.306 mmol, 1.10 eq), 1,4-dioxane (1.2 mL) and acetonitrile (1.2 mL) was stirred at 80° C. for 3 h. After cooling to rt, the solvent was removed under reduced pressure. The crude was purified by RP chromatography (MeCN/H2O 5>95%) to afford 2-[6-(2,2-dimethylpropanoylamino)-2-pyridyl]amino]-N-(3-hydroxy-2,6-dimethyl-phenyl)thiazole-5-carboxamide (71 mg, 0.162 mmol, 58%) as beige solids. ¹H NMR
(300 MHz, Methanol-d4/chloroform -d) δ 8.11 (s, 1H), 7.76-7.51 (m, 2H), 6.90 (d, J=8.2 Hz, 1H), 6.77 (d, J=7.5 Hz, 1H), 6.68 (d, J=8.2 Hz, 1H), 2.17 (s, 3H), 2.13 (s, 3H), 1.32 (s, 9H). MS (ES+) m/z 440.3 (M+H)⁺.

### 2-((6-((tert-Butylsulfinyl)amino)-3-methylpyridin-2-yl)amino)-N-(3-hydroxy-2,6-dimethylphenyl)thiazole-5-carboxamide( ) BAA-340

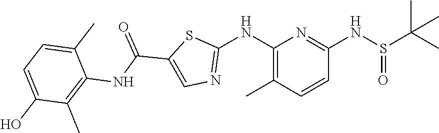 [SMILES: Cc1ccc(NS(=O)C(C)(C)C)nc1Nc1ncc(C(=O)Nc2c(C)ccc(O)c2C)s1]

A mixture of 2-[(6-amino-3-methyl-2-pyridyl)amino]-N-(3-hydroxy-2,6-dimethyl-phenyl)thiazole-5-carboxamide (42 mg, 0.114 mmol, 1.00 eq), t-butylsulfinyl chloride (0.028 mL, 0.227 mmol, 2.00 eq), pyridine (0.046 mL, 0.568 mmol, 5.00 eq), 1,4-dioxane (0.8 mL) and acetonitrile (0.8 mL) was stirred at rt for 5 h before it was diluted with EtOAc (10 mL) and washed with 1 M HCl (10 mL) and sat. NaHCO₃ (10 mL), dried over MgSO₄, filtered and the solvent was removed under reduced pressure. The crude was purified by prep HPLC(low pH) to afford 2-[[6-(tert-butylsulfinylamino)-3-methyl-2-pyridyl]amino]-N-(3-hydroxy-2,6-dimethyl-phenyl)thiazole-5-carboxamide (8.0 mg, 0.0169 mmol, 15%) as beige solids. ¹H NMR (300 MHz, Methanol-d4/chloroform -d) δ 8.10 (s, 1H), 7.40 (d, J=7.9 Hz, 1H), 6.89 (d, J=8.2 Hz, 1H), 6.68 (d, J=8.2 Hz, 1H), 6.50 (d, J=7.9 Hz, 1H), 2.28 (s, 3H), 2.17 (s, 3H), 2.13 (s, 3H), 1.35 (s, 9H). MS (ES+) m/z 474.3 (M+H)⁺.

### 2-((6-Acrylamido-3-methylpyridin-2-yl)amino)-N-(3-hydroxy-2,6-dimethylphenyl)thiazole-5-carboxamide( ) BAA-341

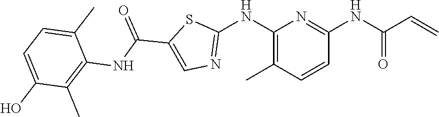 [SMILES: C=CC(=O)Nc1ccc(C)c(Nc2ncc(C(=O)Nc3c(C)ccc(O)c3C)s2)n1]

A mixture of 2-[(6-amino-3-methyl-2-pyridyl)amino]-N-(3-hydroxy-2,6-dimethyl-phenyl)thiazole-5-carboxamide (40 mg, 0.108 mmol, 1.00 eq), acryloyl chloride (0.013 mL, 0.162 mmol, 1.50 eq), pyridine (0.013 mL, 0.162 mmol, 1.50 eq), 1,4-dioxane (0.8 mL) and acetonitrile
(0.8 mL) was stirred at 65° C. for 3 h. After cooling to rt, the mixture was diluted with EtOAc (15 mL), washed with 1 M HCl (2×10 mL) and sat. NaHCO₃ (10 mL), dried over MgSO₄, filtered and the solvent was removed under reduced pressure. The crude was purified by prep HPLC(low pH) to afford N-(3-hydroxy-2,6-dimethyl-phenyl)-2-[[3-methyl-6-(prop-2-enoylamino)-2-pyridyl]amino]thiazole-5-carboxamide (1.2 mg, 0.00283 mmol, 2.6%) as white solids. ¹H NMR (300 MHz, Acetone) δ 9.92 (br, 1H), 8.87 (br, 1H), 8.21 (br, 2H), 7.98 (d, J=8.0 Hz, 1H), 7.65 (d, J=8.1 Hz, 1H), 6.93 (d, J=8.2 Hz, 1H), 6.77 (d, J=8.1 Hz, 1H), 6.69 (dd, J=16.9, 10.1 Hz, 1H), 6.42 (dd, J=16.8, 2.2 Hz, 1H), 5.75 (dd, J=10.1, 2.2 Hz, 1H), 2.46 (s, 3H), 2.19 (s, 3H), 2.16 (s, 3H); [1 H exchanged]. MS (ES+) m/z 424.2 (M+H)⁺.

### N-(3-Hydroxy-2,6-dimethylphenyl)-2-((6-(2-hydroxy-2-methylpropanamido)-3-methylpyridin-2-yl)amino)thiazole-5-carboxamide hydrochloride ( ) BAA-342

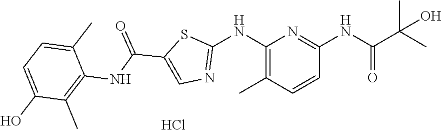 [SMILES: Cc1ccc(NC(=O)C(C)(C)O)nc1Nc1ncc(C(=O)Nc2c(C)ccc(O)c2C)s1 ; Cl]

A mixture of [2-[(6-bromo-5-methyl-2-pyridyl)amino]-1,1-dimethyl-2-oxo-ethyl]acetate (124 mg, 0.395 mmol, 1.30 eq), 2-amino-N-(3-hydroxy-2,6-dimethyl-phenyl)thiazole-5-carboxamide (80 mg, 0.304 mmol, 1.00 eq), tris(dibenzylideneacetone) dipalladium (0)(28 mg, 0.0304 mmol, 0.100 eq), 4,5-bis(diphenylphospheno)-9,9-dimethylxanthene (35 mg, 0.0608 mmol, 0.200 eq), cesium carbonate (110 mg, 0.334 mmol, 1.10 eq) and 1,4-dioxane (2.5 mL) was degassed with nitrogen and stirred at 95° C. for 16 h. After cooling to rt, sodium hydroxide (2 M in H₂O, 2.5 mL) was added and the mixture was stirred at rt for a further 3 h, followed by neutralisation with 2 M HCl and extraction with EtOAc (3×10 mL). The combined organic phases was dried over MgSO₄ and filtered. The solvent was removed under reduced pressure. The crude was purified by RP chromatography (MeCN/H2O 5>95%) and then triturated with DCM to afford the desired compound as a free base. This was dissolved in MeOH(1 mL) and treated with 4 M HCl in dioxane (1 mL) for 1 minute before the solvent was removed under reduced pressure to afford N-(3-hydroxy-2,6-dimethylphenyl)-2-((6-(2-hydroxy-2-methylpropanamido)-3-methylpyridin-2-yl)amino)thiazole-5-carboxamide hydrochloride (45 mg, 0.0915 mmol, 30%) as pink solids. ¹H NMR (300 MHz, Methanol-d4/chloroform -d) δ 8.49 (s, 1H), 7.71 (d, J=8.2 Hz, 1H), 7.51 (br, 1H), 6.86 (d, J=8.3 Hz, 1H), 6.68 (d, J=8.2 Hz, 1H), 2.42 (s, 3H), 2.14 (s, 3H), 2.10 (s, 3H), 1.49 (s, 6H). MS (ES+) m/z 456.2 (M+H)⁺.

### Phenols with NH-Unsubstituted Pyrazole Distal Group

### 2-((4-Bromo-1H-pyrazol-3-yl)amino)-N-(3-hydroxy-2,6-dimethylphenyl)thiazole-5-carboxamide( ) BAA-579

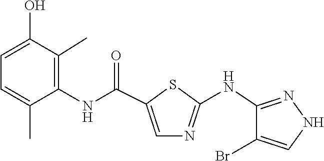 [SMILES: Cc1ccc(O)c(C)c1NC(=O)c1cnc(Nc2n[nH]cc2Br)s1]

tert-Butyl 3-amino-4-bromo-pyrazole-1-carboxylate (140 mg, 0.534 mmol) and 2-bromo-N-[3-[tert-butyl(dimethyl) silyl]oxy-2,6-dimethyl-phenyl]thiazole-5-carboxamide (120 mg, 0.272 mmol) were dissolved in 1,4-dioxane (3 mL). Xantphos (32 mg, 0.0553 mmol) and potassium carbonate 325 mesh (120 mg, 0.868 mmol) were added. The mixture was degassed with nitrogen for 5 minutes, palladium (II)acetate (6.0 mg, 0.0267 mmol) was added, the mixture was degassed with nitrogen for 5 more minutes, then it was heated in a sealed vial at 70° C. for 18 h. The reaction mixture was filtered through celite and evaporated affording crude tert-butyl 4-bromo-3-[5-[3-[tert-butyl(dimethyl) silyl]oxy-2,6-dimethyl-phenyl]carbamoyl]thiazol-2-yl]amino]pyrazole-1-carboxylate
(370 mg, 0.0832 mmol, 30.61% yield) as a brown oil, which was used in the next step without further purification. MS (ES+) m/z=622.3/624.2 (mono Br pattern) [M+H]+.

The product from the first step was (370 mg, 0.0832 mmol) was dissolved in hydrogen chloride 4N in dioxane (1.3 mL, 5.00 mmol). The reaction was stirred at rt overnight, then quenched with sat. aq. NaHCO₃ to neutral, then the solvent was removed in vacuo. The resulting solid was suspended in iPrOH and filtered on celite. The crude product was purified by prep HPLC to afford the title compound (6.3 mg, 0.0153 mmol, 18.36% yield) as a white solid.MS (ES+) m/z=408.1, 410.1 (mono Br pattern) [M+H]+. ¹H NMR (400 MHz, DMSO-d₆) δ 12.91 (s, 1H), 10.71 (s, 1H), 9.48 (s, 1H), 9.16 (s, 1H), 8.05 (s, 1H), 7.97 (s, 1H), 6.87 (d, J=8.1 Hz, 1H), 6.67 (d, J=8.1 Hz, 1H), 2.06 (s, 3H), 1.97 (s, 3H).

### 2-((4-Ethyl-1H-pyrazol-3-yl)amino)-N-(3-hydroxy-2,6-dimethylphenyl)thiazole-5-carboxamide( ) BAA-594

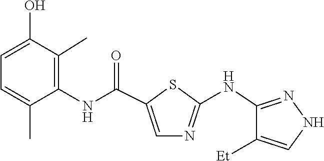 [SMILES: CCc1c[nH]nc1Nc1ncc(C(=O)Nc2c(C)ccc(O)c2C)s1]

4-Ethyl-1-[(4-methoxyphenyl)methyl]pyrazol-3-amine (90 mg, 0.389 mmol) and 2-bromo-N-[3-[tert-butyl(dimethyl) silyl]oxy-2,6-dimethyl-phenyl]thiazole-5-carboxamide (115 mg, 0.260 mmol) were dissolved in 1,4-dioxane (2.5 mL), Xantphos (30 mg, 0.0518 mmol) and potassium carbonate 325 mesh (110 mg, 0.796 mmol) were added, the mixture was degassed with nitrogen for 5 minutes and palladium (II)acetate (6.0 mg, 0.0267 mmol) was added. The mixture was degassed with nitrogen for 5 more minutes, then it was stirred at 100° C. for 16 h in a sealed vial. The mixture was filtered through celite, rinsed with MeOH, the solvent was evaporated in vacuo, and the residue was dissolved in HCl 4N in dioxane (5 mL). The reaction was stirred at rt for 3 days. The solvent was evaporated in vacuo, to afford 2-[4-ethyl-1-[(4-methoxyphenyl)methyl]pyrazol-3-yl]amino]-N-(3-hydroxy-2,6-dimethyl-phenyl)thiazole-5-carboxamide (160 mg, 0.208 mmol, 79.74% yield) as an orange solid. MS (ES+) m/z=478.4 [M+H]+. ¹H NMR (400 MHz, DMSO-d₆) δ 9.63 (s, 1H), 8.19 (s, 1H), 7.58 (s, 1H), 7.29-7.22 (m, 2H), 6.93-6.85 (m, 3H), 6.69 (d, J=8.2 Hz, 1H), 5.14 (s, 2H), 3.71 (s, 3H), 2.44 (q, J=7.6 Hz, 2H), 2.06 (s, 3H), 1.98 (s, 3H), 1.10 (t, J=7.5 Hz, 3H).

This product (100 mg, 0.209 mmol) was dissolved in trifluoroacetic acid (2.5 mL, 32.6 mmol), and was stirred at 80° C. for 18 h in a sealed vial. The solvent was evaporated in vacuo, and the residue was purified by prep HPLC to afford the title compound (26 mg, 0.0739 mmol, 35.27% yield) as a white powder.MS (ES+) m/z=358.3 [M+H]⁺; ¹H NMR (400 MHz, DMSO-d₆) δ 12.22 (s, 1H), 10.62 (s, 1H), 9.43 (s, 1H), 9.15 (s, 1H), 8.08 (s, 1H), 7.50 (s, 1H), 6.87 (d, J=8.1 Hz, 1H), 6.67 (d, J=8.1 Hz, 1H), 2.45 (q, J=7.4 Hz, 2H), 2.06 (s, 3H), 1.98 (s, 3H), 1.11 (t, J=7.5 Hz, 3H).

### 2-((4-Fluoro-1H-pyrazol-3-yl)amino)-N-(3-hydroxy-2,6-dimethylphenyl)thiazole-5-carboxamide( ) BAA-593

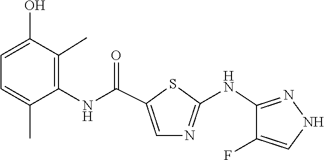 [SMILES: Cc1ccc(O)c(C)c1NC(=O)c1cnc(Nc2n[nH]cc2F)s1]

4-Fluoro-1-[(4-methoxyphenyl)methyl]pyrazol-3-amine (200 mg, 0.904 mmol) and 2-bromo-N-[3-[tert-butyl(dimethyl) silyl]oxy-2,6-dimethyl-phenyl]thiazole-5-carboxamide (200 mg, 0.453 mmol) were dissolved in 1,4-dioxane (5 mL), Xantphos (55 mg, 0.0951 mmol) potassium carbonate 325 mesh (190 mg, 1.37 mmol) and palladium (II)acetate (11 mg, 0.0490 mmol) were added. The mixture was degassed with nitrogen for 5 more minutes, and was stirred at 80° C. for 3 h and 110° C. for 16 h. The mixture was filtered through celite, rinsed with MeOH, the solvent evaporated and the residue was dissolved in HCl 4N in dioxane (5 mL). The reaction was stirred at rt overnight, the solvent was evaporated, the residue was dissolved in chloroform/isopropanol 7:3 (50 mL) and extracted with aq. NaOH 1M (3×50 mL). The combined aqueous extracts were acidified to neutrality with sat. aq. NH₄Cl (approx 50 mL) and extracted with chloroform/isopropanol 7:3 (3×100 mL). The combined organics were dried over Na₂SO₄, filtered and concentrated, to afford 2-[4-fluoro-1-[(4-methoxyphenyl)methyl]pyrazol-3-yl]amino]-N-(3-hydroxy-2,6-dimethyl-phenyl)thiazole-5-carboxamide (199 mg, 0.260 mmol, 57.28% yield) as a brown solid.MS (ES+) m/z=468.2 [M+H]⁺, ¹H NMR (400 MHz, DMSO-d₆) δ9.20 (s, 1H), 8.76 (s, 1H), 7.96 (s, 1H), 7.08 (s, 1H), 6.87 (d, J=8.2 Hz, 1H), 6.84 (d, J=8.8 Hz, 2H), 6.70 (d, J=4.8 Hz, 1H), 6.67 (d, J=8.1 Hz, 2H), 5.09 (s, 2H), 3.70 (s, 3H), 2.07 (s, 3H), 1.99 (s, 3H).

The product from the first step (199 mg, 0.260 mmol) was dissolved in trifluoroacetic acid
(2.5 mL, 32.6 mmol) and was stirred at 80° C. overnight in a sealed vial. The solvent was evaporated and the residue purification by prep HPLC to afford the title compound (14 mg, 0.0400 mmol, 15.42% yield) as a white solid.MS (ES+) m/z=348.3 [M+H]⁺; ¹H NMR (400 MHz, DMSO-d6) δ 12.41 (s, 1H), 10.99 (s, 1H), 9.48 (s, 1H), 9.16 (s, 1H), 8.06 (s, 1H), 7.88 (s, 1H), 6.87 (d, J=8.2 Hz, 1H), 6.67 (d, J=8.2 Hz, 1H), 2.06 (s, 3H), 1.97 (s, 3H). ¹⁹F NMR (376 MHz, DMSO-d₆) δ-184.74.

### NH-modifications on phenols with unsubstituted pyrazole

### 2-((1-(3-Fluorobenzyl)-4-methyl-1H-pyrazol-5-yl)amino)-N-(3-hydroxy-2,6-dimethylphenyl)thiazole-5-carboxamide( ) BAA-375

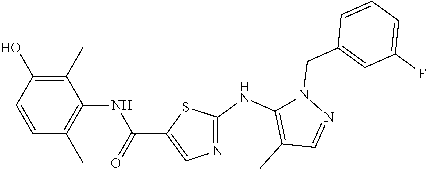 [SMILES: Cc1ccc(O)c(C)c1NC(=O)c1cnc(Nc2c(C)cnn2Cc2cccc(F)c2)s1]

To N-(3-methoxy-2,6-dimethyl-phenyl)-2-[(4-methyl-1H-pyrazol-3-yl)amino]thiazole-5-carboxamide (38 mg, 0.106 mmol, 1 eq) and cesium carbonate (43 mg, 0.133 mmol, 1.25 eq) in CH₂Cl₂ (5 mL) was added dropwise 3-fluorobenzyl bromide (0.014 mL, 0.117 mmol, 1.1 eq) and the reaction heated to 40° C. for 14 h. The reaction was quenched by the addition of sat. aq. NaHCO₃ solution (1 mL). EtOAc (5 mL) and water (5 mL) were added. The phases were separated and the aqueous phase extracted with EtOAc (2×5 mL). Combined organic phases were washed with brine (20 mL), dried over MgSO₄, filtered and concentrated in vacuo. CH₂Cl₂ (5 mL) was added and the mixture cooled to 0° C. Boron tribromide solution (1 M in CH₂Cl2, 0.53 mL, 0.532 mmol, 5 eq) was added dropwise and the reaction allowed to warm to rt for 1 h. The reaction was quenched by the addition of sat. aq. NaHCO₃ solution (1 mL). EtOAc (5 mL) and water (5 mL) were added. The phases were separated and the aqueous phase extracted with EtOAc (2×5 mL). Combined organic phases were washed with NH₄Cl (5 mL, 10% w/w aqueous solution), brine (20 mL), dried over MgSO₄, filtered and concentrated in vacuo. Column chromatography (dichloromethane:methanol) gave 2-[1-[(3-fluorophenyl)methyl]-4-methyl-pyrazol-5-yl]amino]-N-(3-hydroxy-2,6-dimethyl-phenyl)thiazole-5-carboxamide (2.0 mg, 0.00443 mmol, 4.2%). MS (ES+) m/z 452 (M+H). ¹H NMR (300 MHz, MeOD) δ 8.00 (s, 1H), 7.51 (d, J=0.9 Hz, 1H), 7.31 (td, J=7.9, 5.8 Hz, 1H), 7.23-7.08 (m, 2H), 7.04-6.95 (m, 1H), 6.89 (d, J=8.2 Hz, 1H), 6.67 (d, J=8.2 Hz, 1H), 5.14 (s, 2H), 2.13 (s, 3H), 2.08 (s, 3H), 1.79 (d, J=0.8 Hz, 3H). ¹⁹F NMR (282 MHz, MeOD) δ-115.33.

### N-(3-Hydroxy-2,6-dimethylphenyl)-2-((4-methyl-1-(phenylsulfonyl)-1H-pyrazol-3-yl)amino)thiazole-5-carboxamide( ) BAA-379

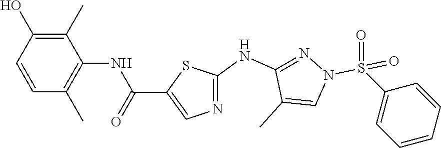 [SMILES: Cc1cn(S(=O)(=O)c2ccccc2)nc1Nc1ncc(C(=O)Nc2c(C)ccc(O)c2C)s1]

To N-(3-methoxy-2,6-dimethyl-phenyl)-2-[(4-methyl-1H-pyrazol-3-yl)amino]thiazole-5-carboxamide (44 mg, 0.123 mmol, 1 eq) and triethylamine (0.021 mL, 0.148 mmol, 1.2 eq) in DCM
(4 mL) at 0° C. was added dropwise benzenesulfonyl chloride (0.017 mL, 0.135 mmol, 1.1 eq) and the reaction stirred at rt for 6 h. CH₂Cl₂ (5 mL) and water (5 mL) were added. The phases were separated and the aqueous phase extracted with CH₂Cl₂ (2×5 mL). Combined organic phases were washed with brine (20 mL), dried over MgSO₄, filtered and concentrated in vacuo. The residue was dissolved in CH₂Cl₂ (6 mL) and boron tribromide solution (0.12 mL, 1.23 mmol, 10 eq) was added in one portion at rt. Upon completion, the CH₂Cl₂ was removed in vacuo, then quenched by the addition of sat. aq. NaHCO₃ solution (5 mL). Ethyl acetate (5 mL) was added. The phases were separated and the aqueous phase extracted with ethyl acetate (2×5 mL). Combined organic phases were washed with brine (20 mL), dried over MgSO₄, filtered and concentrated in vacuo. Column chromatography (CH₂Cl₂: methanol) followed by trituration with CH₂Cl2 gave 2-[1-(benzenesulfonyl)-4-methyl-pyrazol-3-yl]amino]-N-(3-hydroxy-2,6-dimethyl-phenyl)thiazole-5-carboxamide (6.0 mg, 0.0124 mmol, 10%). MS (ES+) m/z 484 (M+H). ¹H NMR
(300 MHz, MeOD) δ 8.09 (s, 1H), 8.06-8.01 (m, 2H), 7.96 (q, J=1.0 Hz, 1H), 7.71-7.62 (m, 1H), 7.58 (ddt, J=8.3, 6.6, 1.5 Hz, 2H), 6.94 (dt, J=8.3, 0.7 Hz, 1H), 6.70 (d, J=8.2 Hz, 1H), 2.24-2.17 (m, 3H), 2.14 (s, 3H), 2.07 (d, J=1.1 Hz, 3H).

### N-(3-hydroxy-2,6-dimethylphenyl)-2-((4-methyl-1-(5-(trifluoromethyl)pyridin-3-yl)-1H-pyrazol-3-yl)amino)thiazole-5-carboxamide( ) BAA-380

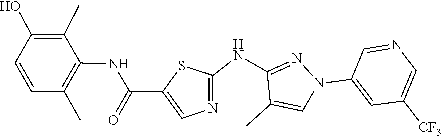 [SMILES: Cc1cn(-c2cncc(C(F)(F)F)c2)nc1Nc1ncc(C(=O)Nc2c(C)ccc(O)c2C)s1]

A mixture of cesium carbonate (49 mg, 0.151 mmol, 1.1 eq), N-(3-methoxy-2,6-dimethyl-phenyl)-2-[(4-methyl-1H-pyrazol-3-yl)amino]thiazole-5-carboxamide (49 mg, 0.137 mmol, 1 eq) and copper (I) iodide (5.2 mg, 0.0274 mmol, 0.2 eq) in DMA (3 mL) was briefly degassed. 3-bromo-5-(trifluoromethyl)pyridine (31 mg, 0.137 mmol, 1 eq) was added and the reaction heated to 120° C. for 2 h under microwave irradiation. Ethyl acetate (5 mL) and water (5 mL) were added. The phases were separated and the aqueous phase extracted with ethyl acetate (2×5 mL). Combined organic phases were washed with NH₄Cl (5 mL, 10% w/w aqueous solution), brine (20 mL), dried over MgSO₄, filtered and concentrated in vacuo. CH₂Cl₂ (6 mL) was added and the mixture cooled to 0° C. Boron tribromide solution (1M in DCM, 0.69 mL, 0.685 mmol, 5 eq) was added dropwise and the reaction stirred for 30 min at 0° C. then 1 h at rt, then concentrated in vacuo. EtOAc (5 mL) and water (5 mL) were added. The phases were separated and the aqueous phase extracted with EtOAc (2×5 mL). Combined organic phases were washed with brine (20 mL), dried over MgSO₄, filtered and concentrated in vacuo. Repeated column chromatography (dichloromethane:methanol) gave N-(3-hydroxy-2,6-dimethyl-phenyl)-2-[4-methyl-1-[5-(trifluoromethyl)-3-pyridyl]pyrazol-3-yl]amino]thiazole-5-carboxamide (1.0 mg, 0.00205 mmol, 1.5%). MS (ES+) m/z 489 (M+H). 1H NMR (300 MHz, MeOD) δ 9.35 (d, J=2.5 Hz, 1H), 8.69 (s, 1H), 8.44 (s, 1H), 8.29 (d, J=1.1 Hz, 1H), 8.13 (s, 1H), 6.93 (d, J=8.2 Hz, 1H), 6.70 (d, J=8.2 Hz, 1H), 2.21 (d, J=1.0 Hz, 3H), 2.18 (d, J=2.1 Hz, 3H), 2.14 (s, 3H). ¹⁹F NMR (282 MHz, MeOD) δ 63.97.

### Phenol Amides and Esters

Amide Formation from Unprotected Phenol BAA-118

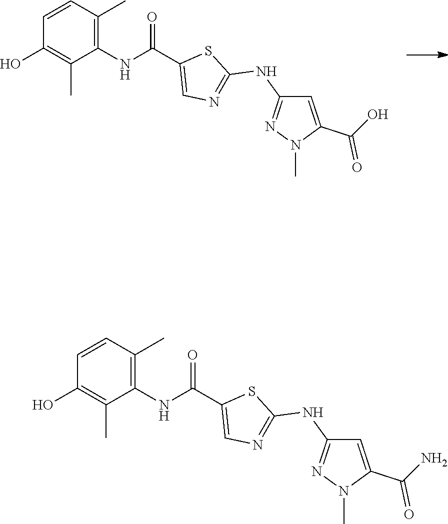 [SMILES: Cc1ccc(O)c(C)c1NC(=O)c1cnc(Nc2cc(C(=O)O)n(C)n2)s1 ; Cc1ccc(O)c(C)c1NC(=O)c1cnc(Nc2cc(C(N)=O)n(C)n2)s1]

### 2-[(5-Carbamoyl-1-methyl-pyrazol-3-yl)amino]-N-(3-hydroxy-2,6-dimethyl-phenyl)thiazole-5-carboxamide (BAA-130)

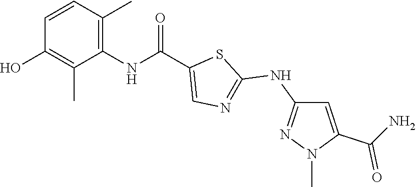 [SMILES: Cc1ccc(O)c(C)c1NC(=O)c1cnc(Nc2cc(C(N)=O)n(C)n2)s1]

A mixture of 5-[5-[(3-hydroxy-2,6-dimethyl-phenyl) carbamoyl]thiazol-2-yl]amino]-2-methyl-pyrazole-3-carboxylic acid (BAA-118)(30 mg, 0.0774 mmol, 1.0 eq), ammonium chloride (21 mg, 0.387 mmol, 5.0 eq), HATU (35 mg, 0.0929 mmol, 1.2 eq) and DIPEA (0.067 mL, 0.387 mmol, 5.0 eq) in DMF (1.5 mL) was stirred at RT for 18 h. Additional ammonium chloride (21 mg, 0.387 mmol, 5.0 eq), HATU (18 mg, 0.0465 mmol, 0.6 eq) and DIPEA (0.067 mL, 0.387 mmol, 5.0 eq) were added and the mixture was stirred at RT for a further 3 h. The mixture was partitioned between EtOAc and water. The aqueous phase was extracted with EtOAc (x 2) and the combined organic layers were washed with brine, dried (MgSO₄), filtered and concentrated under reduced pressure. The crude material was chromatographed (SiO₂) eluting with 0-20% MeOH: DCM to afford the title compound (15 mg, 0.0388 mmol, 50%) as a white solid. MS (ES+) m/z 386.9 (M+H). ¹H NMR (300 MHz, DMSO-d₆) δ 11.19 (s, 1H), 9.54 (s, 1H), 9.20 (s, 1H), 8.11 (s, 1H), 8.06 (s, 1H), 7.55 (s, 1H), 6.89 (d, J=8.2 Hz, 1H), 6.69 (d, J=8.2 Hz, 1H), 6.65 (s, 1H), 4.01 (s, 3H), 2.07 (s, 3H), 1.99 (s, 3H).

Amide Formation from BAA-121

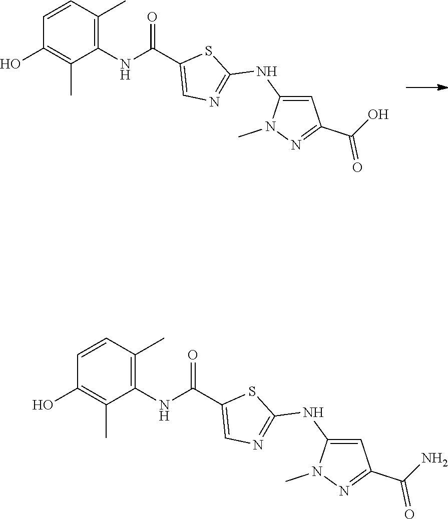 [SMILES: Cc1ccc(O)c(C)c1NC(=O)c1cnc(Nc2cc(C(=O)O)nn2C)s1 ; Cc1ccc(O)c(C)c1NC(=O)c1cnc(Nc2cc(C(N)=O)nn2C)s1]

### 2-[(5-Carbamoyl-2-methyl-pyrazol-3-yl)amino]-N-(3-hydroxy-2,6-dimethyl-phenyl)thiazole-5-carboxamide (BAA-131)

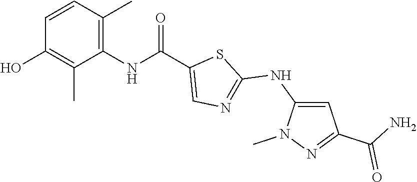 [SMILES: Cc1ccc(O)c(C)c1NC(=O)c1cnc(Nc2cc(C(N)=O)nn2C)s1]

A mixture of 5-[5-[(3-hydroxy-2,6-dimethyl-phenyl) carbamoyl]thiazol-2-yl]amino]-1-methyl-pyrazole-3-carboxylic acid (BAA-121)(15 mg, 0.0387 mmol, 1.0 eq), ammonium chloride (10 mg, 0.194 mmol, 5.0 eq), HATU (22 mg, 0.0581 mmol, 1.5 eq) and DIPEA (0.034 mL, 0.194 mmol, 5.0 eq) in anhydrous DMF (1 mL) under nitrogen was stirred at RT for 18 h. The mixture was partitioned between EtOAc and water. The aqueous phase was extracted with EtOAc (x 3) and the combined organic layers were washed with brine, dried (MgSO₄), filtered and concentrated under reduced pressure. The crude material was chromatographed (SiO₂) eluting with 0-20% MeOH: DCM to afford the title compound BAA-131 (8.0 mg, 0.0207 mmol, 53%) as a white solid. MS (ES+) m/z 386.9 (M+H). ¹H NMR (300 MHz, Methanol-d4) δ 7.97 (s, 1H), 6.93 (d, J=8.2 Hz, 1H), 6.80 (s, 1H), 6.70 (d, J=8.2 Hz, 1H), 3.85 (s, 3H), 2.17 (s, 3H), 2.12 (s, 3H).

Amide Formation from BAA-059 and Other Phenol Pyrazolyl Acetic Acids with Amines

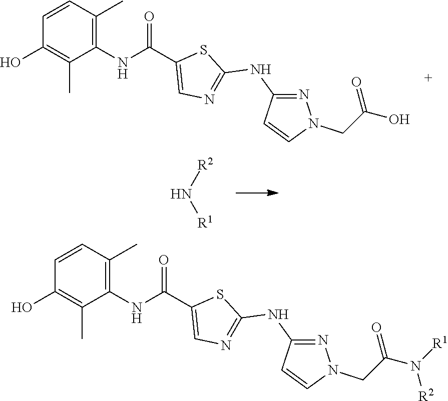

General Method L: Amide Formation Between 2-[3-[[5-[(3-hydroxy-2,6-dimethyl-phenyl) carbamoyl]thiazol-2-yl]amino]pyrazol-1-yl]acetic acid (BAA-059) and amines A stirred mixture of 2-[3-[5-[(3-hydroxy-2,6-dimethyl-phenyl) carbamoyl]thiazol-2-yl]amino]pyrazol-1-yl]acetic acid (BAA-059)(1.0 eq), amine (1.1-5.0 eq), DIPEA (3.0-5.0 eq) and HATU (1.1-1.5 eq) in anhydrous DMF (0.06-0.2 M) was stirred, under nitrogen, at RT. The reaction was monitored by consumption of starting material via UPLC. Additional amine (2.0 eq) and DIPEA (3.0 eq) added if the reaction had stalled. The mixture was concentrated in vacuo, partitioned between EtOAc and water, the organic phase was washed with brine, dried (MgSO₄), filtered, and concentrated in vacuo. The crude material was purified by either normal phase chromatography (SiO₂) using a gradient of EtOAc: PE and/or MeOH: DCM, MeOH(+10% aq. NH3): DCM and/or MeOH: EtOAc, and/or by preparative HPLC-MS using a gradient of high or low pH aq. MeCN.

Where examples are isolated as HCl salts, the free base material was suspended in 1,4-dioxane and HCl (4M in 1,4-dioxane, 20-25 eq) added dropwise at 0° C. then stirred for 1 h. The mixture was concentrated in vacuo, triturated with Et₂O or DCM/Et₂O, filtered, washed with Et₂O and dried in vacuo at 50° C.

### N-(3-Hydroxy-2,6-dimethyl-phenyl)-2-[[1-[2-(4-hydroxy-1-piperidyl)-2-oxo-ethyl]pyrazol-3-yl]amino]thiazole-5-carboxamide (BAA-132)

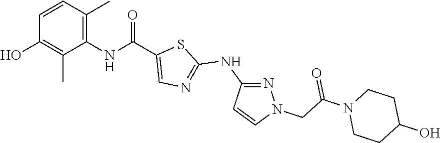 [SMILES: Cc1ccc(O)c(C)c1NC(=O)c1cnc(Nc2ccn(CC(=O)N3CCC(O)CC3)n2)s1]

Prepared as described in Method L from 2-[3-[5-[(3-hydroxy-2,6-dimethyl-phenyl) carbamoyl]thiazol-2-yl]amino]pyrazol-1-yl]acetic acid (BAA-059)(40 mg, 0.103 mmol, 1.0 eq), 4-hydroxypiperidine (13 mg, 0.124 mmol, 1.2 eq), HATU (47 mg, 0.124 mmol, 1.2 eq) and DIPEA (0.054 mL, 0.310 mmol, 3.0 eq) in anhydrous DMF (1 mL) to afford the title compound (25 mg, 0.0531 mmol, 51%) as a white solid after normal phase chromatography (SiO₂) eluting with 0-30% MeOH: DCM. MS (ES-) m/z 469.0 (M−H). ¹H NMR (300 MHz, DMSO-d₆) δ 11.04 (s, 1H), 9.51 (s, 1H), 9.19 (s, 1H), 8.10 (s, 1H), 7.60 (d, J=2.4 Hz, 1H), 6.88 (d, J=8.2 Hz, 1H), 6.68 (d, J=8.2 Hz, 1H), 5.98 (d, J=2.4 Hz, 1H), 5.05 (s, 2H), 4.78 (d, J=4.1 Hz, 1H), 3.91-3.81 (m, 1H), 3.78-3.65 (m, 2H), 3.29-3.17 (m, 1H), 3.13-2.99 (m, 1H), 2.07 (s, 3H), 1.99 (s, 3H), 1.82-1.65 (m, 2H), 1.45-1.21 (m, 2H).

The following example compounds were prepared similarly using Method L using the appropriately substituted amine:

| # | Structure & Name | Analytical Data |
|---|---|---|
| BAA- 133 | 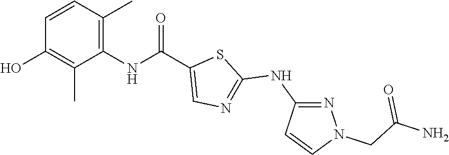 [SMILES: Cc1ccc(O)c(C)c1NC(=O)c1cnc(Nc2ccn(CC(N)=O)n2)s1] | MS (ES+) m/z 386.9 (M + H). ¹H NMR (300 MHz, DMSO-d₆) δ 11.08 (s, 1H), 9.52 (s, 1H), 9.19 (s, 1H), 8.10 (s, 1H), 7.64 (d, J = 2.4 Hz, 1H), 7.48 (s, 1H), 7.29 (s, 1H), 6.89 (d, J = 8.2 Hz, 1H), 6.68 (d, J = 8.2 Hz, 1H), 5.98 (d, J = 2.4 Hz, 1H), 4.70 (s, 2H), 2.07 (s, 3H), 1.99 (s, 3H). |
| | 2-[[1-(2-amino-2-oxo-ethyl) pyrazol-3- | |
| | yl]amino]-N-(3-hydroxy-2,6-dimethyl- | |
| | phenyl) thiazole-5-carboxamide | |
| BAA- 134 | 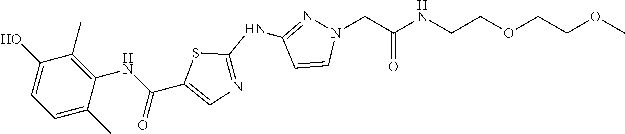 [SMILES: COCCOCCNC(=O)Cn1ccc(Nc2ncc(C(=O)Nc3c(C)ccc(O)c3C)s2)n1] | MS (ES+) m/z 489.4 (M + H). ¹H NMR (300 MHz, Methanol-d₄) δ 8.05 (s, 1H), 7.61 (d, J = 2.4 Hz, 1H), 6.91 (dt, J = 8.2, 0.7 Hz, 1H), 6.68 (d, J = 8.2 Hz, 1H), 6.08 (d, J = 2.4 Hz, 1H), 4.80 (s, 2H), 3.61-3.49 (m, 4H), 3.49-3.33 (m, 4H), 3.30 (s, 4H), 2.16 (s, 3H), 2.11 (s, 3H). |
| | N-(3-hydroxy-2,6-dimethyl-phenyl)-2- | |
| | [[1-[2-[2-(2- | |
| | methoxyethoxy)ethylamino]-2-oxo- | |
| | ethyl]pyrazol-3-yl]amino]thiazole-5- | |
| | carboxamide | |
| BAA- 135 | 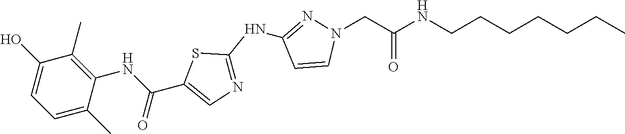 [SMILES: CCCCCCCNC(=O)Cn1ccc(Nc2ncc(C(=O)Nc3c(C)ccc(O)c3C)s2)n1] | MS (ES+) m/z 485.4 (M + H). ¹H NMR (300 MHz, DMSO-d₆) δ 11.06 (s, 1H), 9.49 (s, 1H), 9.18 (s, 1H), 8.09 (s, 1H), 8.04 (t, J = 5.6 Hz, 1H), 7.64 (d, J = 2.4 Hz, 1H), 6.87 (d, J = 8.2 Hz, 1H), 6.67 (d, J = 8.2 Hz, 1H), 5.97 (d, J = 2.3 Hz, 1H), 4.69 (s, 2H), 3.06 (dt, J = 7.4, 4.1 Hz, 2H), 2.06 (s, 3H), 1.98 (s, 3H), 1.40 (t, J = 6.5 Hz, 2H), 1.27-1.16 (m, 9H), 0.85-0.78 (m, 3H). |
| | 2-[1-[2-(heptylamino)-2-oxo- | |
| | ethyl]pyrazol-3-yl]amino]-N-(3- | |
| | hydroxy-2,6-dimethyl-phenyl)thiazole- | |
| | 5-carboxamide | |
| BAA- 136 | 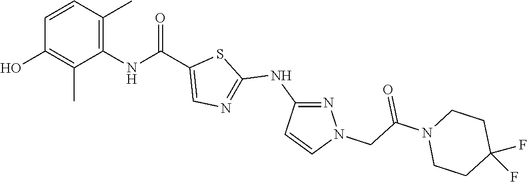 [SMILES: Cc1ccc(O)c(C)c1NC(=O)c1cnc(Nc2ccn(CC(=O)N3CCC(F)(F)CC3)n2)s1] | MS (ES+) m/z 490.9 (M + H). ¹H NMR (300 MHz, Methanol-d₄) δ 8.06 (s, 1H), 7.57 (d, J = 2.4 Hz, 1H), 6.93 (d, J = 8.2 Hz, 1H), 6.70 (d, J = 8.2 Hz, 1H), 6.11 (d, J = 2.4 Hz, 1H), 5.14 (s, 2H), 3.74 (t, J = 5.9 Hz, 4H), 2.18 (s, 3H), 2.13 (s, 3H), 2.11-1.93 (m, 4H). ¹⁹F NMR (282 MHz, Methanol-d₄) δ −99.38. |
| | 2-[[ 1-[2-(4,4-difluoro-1-piperidyl)-2- | |
| | oxo-ethyl]pyrazol-3-yl]amino]-N-(3- | |
| | hydroxy-2,6-dimethyl-phenyl)thiazole- | |
| | 5-carboxamide | |
| BAA- 137 | 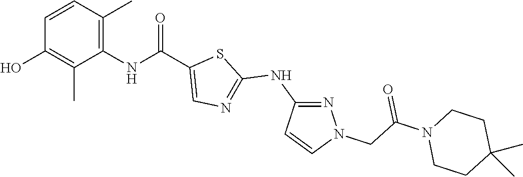 [SMILES: Cc1ccc(O)c(C)c1NC(=O)c1cnc(Nc2ccn(CC(=O)N3CCC(C)(C)CC3)n2)s1] | MS (ES+) m/z 483.0 (M + H). ¹H NMR (300 MHz, Methanol-d₄) δ 8.06 (s, 1H), 7.56 (d, J = 2.4 Hz, 1H), 6.93 (d, J = 8.2 Hz, 1H), 6.70 (d, J = 8.2 Hz, 1H), 6.09 (d, J = 2.4 Hz, 1H), 5.06 (s, 2H), 3.62-3.54 (m, 4H), 2.17 (s, 3H), 2.13 (s, 3H), 1.45-1.34 (m, 4H), 1.00 (s, 6H). |
| | 2-[1-[2-(4,4-dimethyl-1-piperidyl)-2- | |
| | oxo-ethyl]pyrazol-3-yl]amino]-N-(3- | |
| | hydroxy-2,6-dimethyl-phenyl)thiazole- | |
| | 5-carboxamide | |
| BAA- 138 | 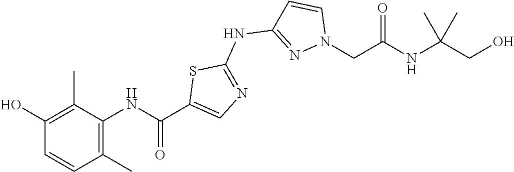 [SMILES: Cc1ccc(O)c(C)c1NC(=O)c1cnc(Nc2ccn(CC(=O)NC(C)(C)CO)n2)s1] | MS (ES+) m/z 459.0 (M + H). ¹H NMR (300 MHz, Methanol-d₄) δ 8.07 (s, 1H), 7.60 (d, J = 2.4 Hz, 1H), 6.93 (d, J = 8.2 Hz, 1H), 6.70 (d, J = 8.2 Hz, 1H), 6.10 (d, J = 2.4 Hz, 1H), 4.75 (s, 2H), 3.59 (s, 2H), 2.18 (s, 3H), 2.13 (s, 3H), 1.31 (s, 6H). |
| | 2-[1-[2-[(2-hydroxy-1, 1-dimethyl- | |
| | ethyl)amino]-2-oxo-ethyl]pyrazol-3- | |
| | ylJamino]-N-(3-hydroxy-2,6-dimethyl- | |
| | phenyl)thiazole-5-carboxamide | |
| BAA- 139 | 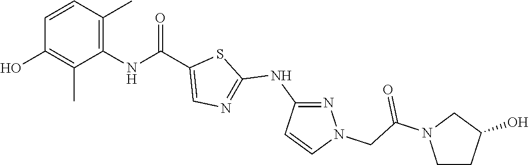 [SMILES: Cc1ccc(O)c(C)c1NC(=O)c1cnc(Nc2ccn(CC(=O)N3CC[C@@H](O)C3)n2)s1] | MS (ES+) m/z 457.0 (M + H). ¹H NMR (300 MHz, Methanol-d₄) δ 8.05 (s, 1H), 7.58 (d, J = 2.4 Hz, 1H), 6.93 (d, J = 8.2 Hz, 1H), 6.70 (d, J = 8.2 Hz, 1H), 6.09 (dd, J = 2.4, 0.9 Hz, 1H), 5.05-4.93 (m, 2H), 4.57-4.40 (m, 1H), 3.92-3.47 (m, 4H), 2.18 (s, 3H), 2.13 (s, 3H), 2.12-1.87 (m, 2H). |
| | N-(3-hydroxy-2,6-dimethyl-phenyl)-2- | |
| | [[1-[2-[(3R)-3-hydroxypyrrolidin-1-yl]- | |
| | 2-oxo-ethyl]pyrazol-3- | |
| | yl]amino]thiazole-5-carboxamide | |
| BAA- 140 | 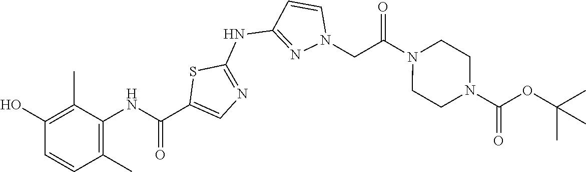 [SMILES: Cc1ccc(O)c(C)c1NC(=O)c1cnc(Nc2ccn(CC(=O)N3CCN(C(=O)OC(C)(C)C)CC3)n2)s1] | MS (ES+) m/z 556.0 (M + H). ¹H NMR (300 MHz, DMSO-d₆) δ 11.06 (s, 1H), 9.51 (s, 1H), 9.19 (s, 1H), 8.10 (s, 1H), 7.59 (d, J = 2.4 Hz, 1H), 6.88 (d, J = 8.2 Hz, 1H), 6.68 (d, J = 8.1 Hz, 1H), 5.99 (d, J = 2.4 Hz, 1H), 5.09 (s, 2H), 3.57-3.41 (m, 4H), 2.07 (s, 3H), 1.98 (s, 3H), 1.41 (s, 9H). ¹H NMR (300 MHz, DMSO-d₆ + D₂O) δ 8.05 (d, J = 2.2 Hz, 1H), 7.56 (d, J = 2.4 Hz, 1H), 6.88 (d, J = 8.3 Hz, 1H), 6.66 (d, J = 8.2 Hz, 1H), 6.03 (d, J = 2.3 Hz, 1H), 5.03 (s, 2H), 3.61- |
| | | 3.14 (m, 8H), 2.03 (s, 3H), 1.94 (s, 3H), 1.35 (s, |
| | tert-butyl 4-[2-[3-[5-[(3-hydroxy-2,6- | 9H). |
| | dimethyl-phenyl)carbamoyl]thiazol-2- | |
| | ylamino]pyrazol-1- | |
| | yl]acetyl]piperazine-1-carboxylate | |
| BAA- 141 | 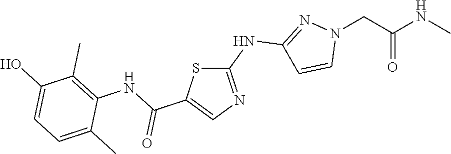 [SMILES: CNC(=O)Cn1ccc(Nc2ncc(C(=O)Nc3c(C)ccc(O)c3C)s2)n1] | MS (ES+) m/z 400.9 (M + H). ¹H NMR (300 MHz, DMSO-d₆) δ 11.08 (s, 1H), 9.52 (s, 1H), 9.19 (s, 1H), 8.10 (s, 1H), 8.00 (q, J = 4.5 Hz, 1H), 7.65 (d, J = 2.4 Hz, 1H), 6.89 (d, J = 8.2 Hz, 1H), 6.68 (d, J = 8.2 Hz, 1H), 6.00 (d, J = 2.4 Hz, 1H), 4.70 (s, 2H), 2.63 (d, J = 4.6 Hz, 3H), 2.07 (s, 3H), 1.99 (s, 3H). |
| | N-(3-hydroxy-2,6-dimethyl-phenyl)-2- | |
| | [[1-[2-(methylamino)-2-oxo- | |
| | ethyl]pyrazol-3-yl]amino]thiazole-5- | |
| | carboxamide | |
| BAA- 142 | 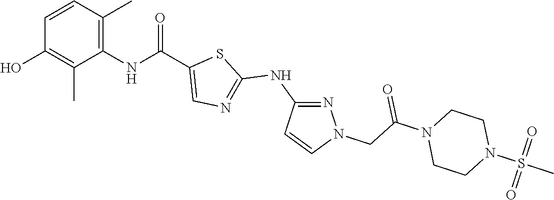 [SMILES: Cc1ccc(O)c(C)c1NC(=O)c1cnc(Nc2ccn(CC(=O)N3CCN(S(C)(=O)=O)CC3)n2)s1] | MS (ES+) m/z 533.9 (M + H). ¹H NMR (300 MHz, DMSO-d₆) δ 11.07 (s, 1H), 9.52 (s, 1H), 9.19 (s, 1H), 8.10 (s, 1H), 7.59 (d, J = 2.4 Hz, 1H), 6.88 (d, J = 8.2 Hz, 1H), 6.68 (d, J = 8.2 Hz, 1H), 5.99 (d, J = 2.4 Hz, 1H), 5.11 (s, 2H), 3.72-3.52 (m, 4H), 3.24-3.08 (m, 4H), 2.89 (s, 3H), 2.07 (s, 3H), 1.98 (s, 3H). |
| | N-(3-hydroxy-2,6-dimethyl-phenyl)-2- | |
| | [1-[2-(4-methylsulfonylpiperazin-1- | |
| | yl)-2-oxo-ethyl]pyrazol-3- | |
| | yl]amino]thiazole-5-carboxamide | |
| BAA- 143 | 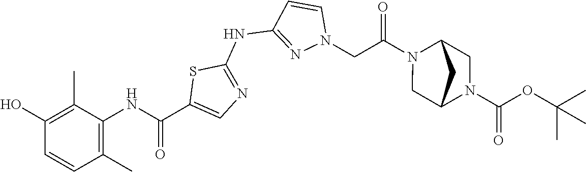 [SMILES: Cc1ccc(O)c(C)c1NC(=O)c1cnc(Nc2ccn(CC(=O)N3C[C@@H]4C[C@H]3CN4C(=O)OC(C)(C)C)n2)s1] | MS (ES+) m/z 567.9 (M + H). ¹H NMR (300 MHz, DMSO-d₆) δ 11.06 (d, J = 5.2 Hz, 1H), 9.51 (s, 1H), 9.19 (s, 1H), 8.10 (s, 1H), 7.72-7.56 (m, 1H), 6.89 (d, J = 8.2 Hz, 1H), 6.68 (d, J = 8.2 Hz, 1H), 6.05-5.94 (m, 1H), 5.20-4.66 (m, 3H), 4.53-4.28 (m, 1H), 3.66-3.09 (m, 4H), 2.07 (s, 3H), 1.99 (s, 3H), 1.95-1.74 (m, 2H), 1.50-1.28 (m, 9H). ¹H NMR (300 MHz, DMSO-d₆ + D₂O) δ 8.04 (s, 1H), 7.72-7.50 (m, 1H), 6.89 (d, J = 8.3 Hz, 1H), 6.67 (d, J = 8.2 Hz, 1H), 6.10-5.94 (m, |
| | | 1H), 5.11-4.61 (m, 3H), 4.46-4.33 (m, 1H), |
| | tert-butyl (1S,4S)-5-[2-[3-[5-[(3- | 3.62-3.04 (m, 4H), 2.03 (s, 3H), 1.95 (s, 3H), |
| | hydroxy-2,6-dimethyl- | 1.91-1.72 (m, 2H), 1.41-1.18 (m, 9H). |
| | phenyl)carbamoyl]thiazol-2- | |
| | yl]amino]pyrazol-1-yl]acetyl]-2,5- | |
| | diazabicyclo[2.2. 1 ]heptane-2- | |
| | carboxylate | |
| BAA- 144 | 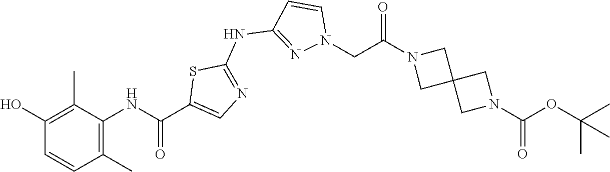 [SMILES: Cc1ccc(O)c(C)c1NC(=O)c1cnc(Nc2ccn(CC(=O)N3CC4(C3)CN(C(=O)OC(C)(C)C)C4)n2)s1] | MS (ES+) m/z 567.9 (M + H). ¹H NMR (300 MHz, DMSO-d₆) δ 11.08 (s, 1H), 9.52 (s, 1H), 9.19 (s, 1H), 8.11 (s, 1H), 7.61 (d, J = 2.4 Hz, 1H), 6.89 (d, J = 8.2 Hz, 1H), 6.69 (d, J = 8.2 Hz, 1H), 5.99 (d, J = 2.3 Hz, 1H), 4.75 (s, 2H), 4.28 (s, 2H), 4.05 (s, 2H), 3.98 (s, 4H), 2.07 (s, 3H), 1.99 (s, 3H), 1.36 (s, 9H). |
| | tert-butyl 6-[2-[3-[[5-[(3-hydroxy-2,6- | |
| | dimethyl-phenyl)carbamoyl]thiazol-2- | |
| | yl]amino]pyrazol-1-yl]acetyl]-2,6- | |
| | diazaspiro[3.3]heptane-2-carboxylate | |
| BAA- 145 | 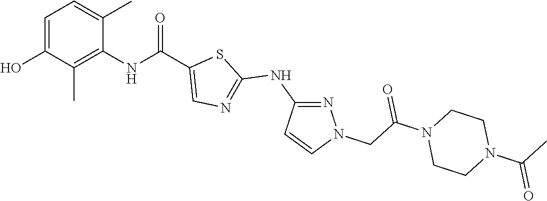 [SMILES: CC(=O)N1CCN(C(=O)Cn2ccc(Nc3ncc(C(=O)Nc4c(C)ccc(O)c4C)s3)n2)CC1] | MS (ES+) m/z 498.0 (M + H). ¹H NMR (300 MHz, DMSO-d₆) δ 11.06 (s, 1H), 9.52 (s, 1H), 9.19 (s, 1H), 8.10 (s, 1H), 7.60 (d, J = 2.3 Hz, 1H), 6.88 (d, J = 8.2 Hz, 1H), 6.68 (d, J = 8.2 Hz, 1H), 5.99 (d, J = 2.3 Hz, 1H), 5.11 (d, J = 3.6 Hz, 2H), 3.62-3.41 (m, 8H), 2.07 (s, 3H), 2.03 (d, J = 3.9 Hz, 3H), 1.98 (s, 3H). |
| | 2-[ 1-[2-(4-acetylpiperazin-1-yl)-2- | |
| | oxo-ethyl]pyrazol-3-yl]amino]-N-(3- | |
| | hydroxy-2,6-dimethyl-phenyl)thiazole- | |
| | 5-carboxamide | |
| BAA- 146 | 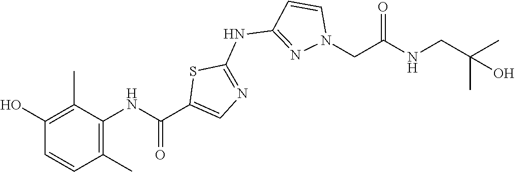 [SMILES: Cc1ccc(O)c(C)c1NC(=O)c1cnc(Nc2ccn(CC(=O)NCC(C)(C)O)n2)s1] | MS (ES+) m/z 459.0 (M + H). ¹H NMR (300 MHz, DMSO-d₆) δ 11.07 (s, 1H), 9.51 (s, 1H), 9.19 (s, 1H), 8.10 (s, 1H), 7.90 (t, J = 5.9 Hz, 1H), 7.67 (d, J = 2.3 Hz, 1H), 6.89 (d, J = 8.2 Hz, 1H), 6.69 (d, J = 8.2 Hz, 1H), 5.99 (d, J = 2.3 Hz, 1H), 4.79 (s, 2H), 4.49 (s, 1H), 3.06 (d, J = 5.9 Hz, 2H), 2.07 (s, 3H), 1.99 (s, 3H), 1.05 (s, 6H). |
| | N-(3-hydroxy-2,6-dimethyl-phenyl)-2- | |
| | [1-[2-[(2-hydroxy-2-methyl- | |
| | propyl)amino]-2-oxo-ethyl]pyrazol-3- | |
| | yl]amino]thiazole-5-carboxamide | |
| BAA- 147 | 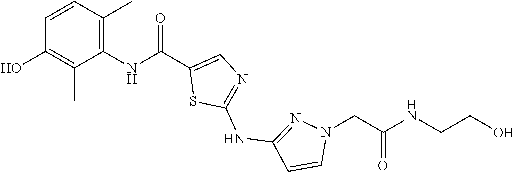 [SMILES: Cc1ccc(O)c(C)c1NC(=O)c1cnc(Nc2ccn(CC(=O)NCCO)n2)s1] | MS (ES+) m/z 430.9 (M + H). ¹H NMR (300 MHz, Methanol-d₄) δ 8.06 (s, 1H), 7.63 (d, J = 2.4 Hz, 1H), 6.93 (d, J = 8.2 Hz, 1H), 6.70 (d, J = 8.2 Hz, 1H), 6.11 (d, J = 2.4 Hz, 1H), 4.82 (s, 2H), 3.64 (t, J = 5.7 Hz, 2H), 3.37 (t, J = 5.7 Hz, 2H), 2.18 (s, 3H), 2.13 (s, 3H). |
| | N-(3-hydroxy-2,6-dimethyl-phenyl)-2- | |
| | [[1-[2-(2-hydroxyethylamino)-2-oxo- | |
| | ethyl]pyrazol-3-yl]amino]thiazole-5- | |
| | carboxamide | |
| BAA- 148 | 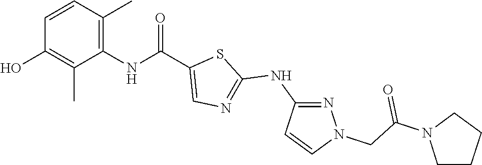 [SMILES: Cc1ccc(O)c(C)c1NC(=O)c1cnc(Nc2ccn(CC(=O)N3CCCC3)n2)s1] | MS (ES+) m/z 441.0 (M + H). ¹H NMR (300 MHz, DMSO-d₆) δ 11.06 (s, 1H), 9.51 (s, 1H), 9.19 (s, 1H), 8.10 (s, 1H), 7.60 (d, J = 2.4 Hz, 1H), 6.88 (d, J = 8.2 Hz, 1H), 6.68 (d, J = 8.2 Hz, 1H), 5.98 (d, J = 2.4 Hz, 1H), 4.94 (s, 2H), 3.53 (t, J = 6.7 Hz, 2H), 3.32 (t, J = 6.8 Hz, 2H), 2.07 (s, 3H), 1.99 (s, 3H), 1.92 (p, J = 6.5 Hz, 2H), 1.78 (p, J = 6.4 Hz, 2H). |
| | N-(3-hydroxy-2,6-dimethyl-phenyl)-2- | |
| | [[1-(2-oxo-2-pyrrolidin-1-yl- | |
| | ethyl)pyrazol-3-yl]amino]thiazole-5- | |
| | carboxamide | |
| BAA- 149 | 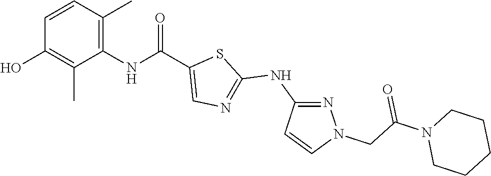 [SMILES: Cc1ccc(O)c(C)c1NC(=O)c1cnc(Nc2ccn(CC(=O)N3CCCCC3)n2)s1] | MS (ES+) m/z 455.0 (M + H). ¹H NMR (300 MHz, DMSO-d₆) δ 11.04 (s, 1H), 9.51 (s, 1H), 9.19 (s, 1H), 8.10 (s, 1H), 7.60 (d, J = 2.3 Hz, 1H), 6.88 (d, J = 8.2 Hz, 1H), 6.68 (d, J = 8.2 Hz, 1H), 5.98 (d, J = 2.4 Hz, 1H), 5.04 (s, 2H), 3.53-3.37 (m, 4H), 2.07 (s, 3H), 1.99 (s, 3H), 1.64-1.50 (m, 4H), 1.49-1.41 (m, 2H). |
| | N-(3-hydroxy-2,6-dimethyl-phenyl)-2- | |
| | [[1-[2-oxo-2-(1-piperidyl)ethyl]pyrazol- | |
| | 3-yl]amino]thiazole-5-carboxamide | |
| BAA- 150 | 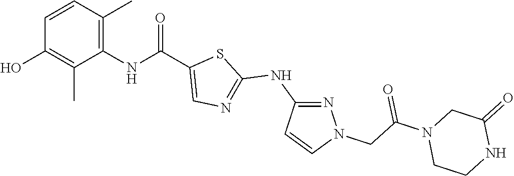 [SMILES: Cc1ccc(O)c(C)c1NC(=O)c1cnc(Nc2ccn(CC(=O)N3CCNC(=O)C3)n2)s1] | MS (ES+) m/z 469.9 (M + H). ¹H NMR (300 MHz, Methanol-d₄) δ 8.05 (d, J = 4.6 Hz, 1H), 7.59 (t, J = 2.7 Hz, 1H), 6.93 (d, J = 8.3 Hz, 1H), 6.70 (d, J = 8.2 Hz, 1H), 6.10 (d, J = 2.4 Hz, 1H), 5.12 (d, J = 12.7 Hz, 2H), 4.36 (s, 1H), 4.18 (s, 1H), 3.93 (t, J = 5.4 Hz, 1H), 3.82 (t, J = 5.4 Hz, 1H), 3.45 (t, J = 5.4 Hz, 1H), 3.40-3.35 (m, 1H), 2.18 (s, 3H), 2.13 (s, 3H). |
| | N-(3-hydroxy-2,6-dimethyl-phenyl)-2- | |
| | [1-[2-oxo-2-(3-oxopiperazin-1- | |
| | yl)ethyl]pyrazol-3-yl]amino]thiazole-5- | |
| | carboxamide | |
| BAA- 151 | 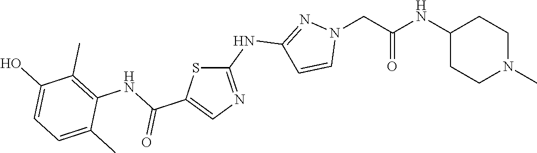 [SMILES: Cc1ccc(O)c(C)c1NC(=O)c1cnc(Nc2ccn(CC(=O)NC3CCN(C)CC3)n2)s1] | MS (ES+) m/z 484.0 (M + H). ¹H NMR (300 MHz, DMSO-d₆) δ 11.42-11.02 (br s, 1H), 10.39-10.19 (br s, 1H), 9.59 (s, 1H), 9.40-9.00 (br s, 1H), 8.47 (d, J = 7.4 Hz, 1H), 8.15 (s, 1H), 7.66 (d, J = 2.3 Hz, 1H), 6.88 (d, J = 8.2 Hz, 1H), 6.70 (d, J = 8.2 Hz, 1H), 6.00 (d, J = 2.4 Hz, 1H), 4.71 (s, 2H), 3.94- 3.63 (m, 1H), 3.42-3.27 (m, 2H), 3.02 (q, J = 11.7 Hz, 2H), 2.80-2.62 (m, 3H), 2.07 (s, 3H), 2.03-1.90 (m, 5H), 1.90-1.64 (m, 2H). |
| | | 1H NMR (300 MHz, DMSO-d6 + D2O) 8.06 (s, |
| | N-(3-hydroxy-2,6-dimethyl-phenyl)-2- | 1H), 7.60 (d, J = 2.5 Hz, 1H), 6.89 (d, J = 8.3 |
| | [1-[2-[(1-methyl-4-piperidyl)amino]-2- | Hz, 1H), 6.67 (d, J = 8.2 Hz, 1H), 6.01 (d, J = |
| | oxo-ethyl]pyrazol-3-yl]amino]thiazole- | 2.4 Hz, 1H), 4.68 (s, 2H), 3.83-3.67 (m, 1H), |
| | 5-carboxamide (hydrochloride) | 3.42-3.21 (m, 2H), 3.13-2.89 (m, 2H), 2.69 |
| | | (s, 3H), 2.07-1.85 (m, 8H), 1.77-1.46 (m, |
| | | 2H). |
| BAA- 152 | 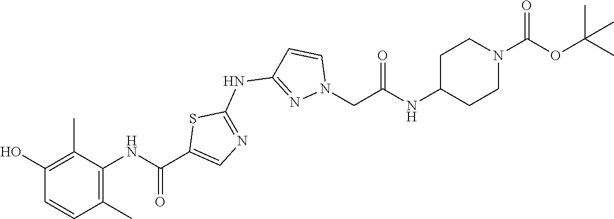 [SMILES: Cc1ccc(O)c(C)c1NC(=O)c1cnc(Nc2ccn(CC(=O)NC3CCN(C(=O)OC(C)(C)C)CC3)n2)s1] | MS (ES+) m/z 570.0 (M + H). ¹H NMR (300 MHz, Methanol-d₄) δ 8.06 (s, 1H), 7.61 (d, J = 2.4 Hz, 1H), 6.93 (d, J = 8.2 Hz, 1H), 6.70 (d, J = 8.2 Hz, 1H), 6.08 (d, J = 2.4 Hz, 1H), 4.77 (s, 2H), 4.06-3.95 (m, 2H), 3.95 -3.83 (m, 1H), 2.98-2.83 (m, 2H), 2.18 (s, 3H), 2.13 (s, 3H), 1.93-1.84 (m, 2H), 1.49- 1.39 (m, 11H). |
| | tert-butyl 4-[2-[3-[5-[(3-hydroxy-2,6- | |
| | dimethyl-phenyl)carbamoyl]thiazol-2- | |
| | yl]amino]pyrazol-1- | |
| | yllacetyl]amino]piperidine-1- | |
| | carboxylate | |
| BAA- 153 | 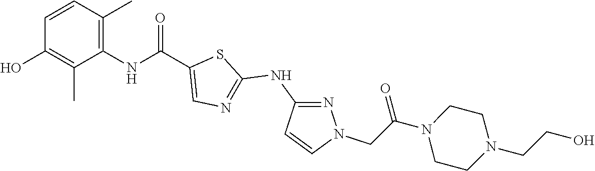 [SMILES: Cc1ccc(O)c(C)c1NC(=O)c1cnc(Nc2ccn(CC(=O)N3CCN(CCO)CC3)n2)s1] | MS (ES+) m/z 499.9 (M + H). ¹H NMR (300 MHz, Methanol-d₄) δ 8.05 (s, 1H), 7.58 (d, J = 2.4 Hz, 1H), 6.93 (d, J = 8.3 Hz, 1H), 6.70 (d, J = 8.2 Hz, 1H), 6.09 (d, J = 2.4 Hz, 1H), 5.09 (s, 2H), 3.75-3.62 (m, 6H), 2.65- 2.52 (m, 6H), 2.18 (s, 3H), 2.13 (s, 3H). |
| | N-(3-hydroxy-2,6-dimethyl-phenyl)-2- | |
| | [[1-[2-[4-(2-hydroxyethyl) piperazin-1- | |
| | yl]-2-oxo-ethyl]pyrazol-3- | |
| | ylamino]thiazole-5-carboxamide | |
| BAA- 154 | 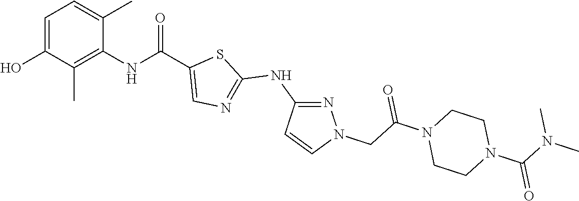 [SMILES: Cc1ccc(O)c(C)c1NC(=O)c1cnc(Nc2ccn(CC(=O)N3CCN(C(=O)N(C)C)CC3)n2)s1] | MS (ES+) m/z 526.9 (M + H). ¹H NMR (300 MHz, Methanol-d₄) δ 8.06 (s, 1H), 7.58 (d, J = 2.4 Hz, 1H), 6.93 (d, J = 8.2 Hz, 1H), 6.70 (d, J = 8.2 Hz, 1H), 6.10 (d, J = 2.4 Hz, 1H), 5.10 (s, 2H), 3.74-3.60 (m, 4H), 3.32- 3.22 (m, 4H), 2.86 (s, 6H), 2.17 (s, 3H), 2.13 (s, 3H). |
| | 2-[ 1-[2-[4- | |
| | (dimethylcarbamoyl)piperazin-1-yl]-2- | |
| | oxo-ethyl]pyrazol-3-yl]amino]-N-(3- | |
| | hydroxy-2,6-dimethyl-phenyl)thiazole- | |
| | 5-carboxamide | |
| BAA- 155 | 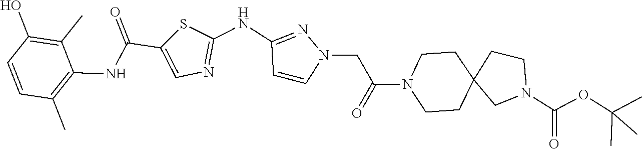 [SMILES: Cc1ccc(O)c(C)c1NC(=O)c1cnc(Nc2ccn(CC(=O)N3CCC4(CC3)CCN(C(=O)OC(C)(C)C)C4)n2)s1] | MS (ES+) m/z 609.9 (M + H). ¹H NMR (300 MHz, Methanol-d₄) δ 8.06 (s, 1H), 7.57 (d, J = 2.4 Hz, 1H), 6.93 (d, J = 8.2 Hz, 1H), 6.70 (d, J = 8.2 Hz, 1H), 6.09 (d, J = 2.4 Hz, 1H), 5.16-5.01 (m, 2H), 3.75-3.49 (m, 4H), 3.44-3.36 (m, 2H), 3.22 (s, 2H), 2.18 (s, 3H), 2.13 (s, 3H), 1.82 (t, J = 7.2 Hz, 2H), 1.63 (t, J = 5.6 Hz, 2H), 1.57 (t, J = 5.8 Hz, 2H), 1.47 (s, 9H). |
| | tert-butyl 8-[2-[3-[5-[(3-hydroxy-2,6- | |
| | dimethyl-phenyl)carbamoyl]thiazol-2- | |
| | ylamino]pyrazol-1-yl]acetyl]-2,8- | |
| | diazaspiro[4.5]decane-2-carboxylate | |
| BAA- 156 | 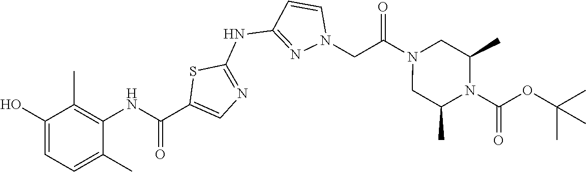 [SMILES: Cc1ccc(O)c(C)c1NC(=O)c1cnc(Nc2ccn(CC(=O)N3C[C@@H](C)N(C(=O)OC(C)(C)C)[C@@H](C)C3)n2)s1] | MS (ES+) m/z 583.9 (M + H). ¹H NMR (300 MHz, Methanol-d₄) δ 8.05 (s, 1H), 7.63 (d, J = 2.4 Hz, 1H), 6.93 (d, J = 8.2 Hz, 1H), 6.70 (d, J = 8.2 Hz, 1H), 6.11 (d, J = 2.4 Hz, 1H), 5.15 (s, 2H), 4.34 (d, J = 13.3 Hz, 1H), 4.31-4.19 (m, 2H), 3.96 (d, J = 13.9 Hz, 1H), 3.46 (dd, J = 13.9, 4.6 Hz, 1H), 2.96 (dd, J = 13.2, 4.7 Hz, 1H), 2.18 (s, 3H), 2.13 (s, 3H), 1.49 (s, 9H), 1.31 (d, J = 6.9 Hz, 3H), 1.21 (d, J = 6.9 Hz, 3H). |
| | tert-butyl (2S,6R)-4-[2-[3-[5-[(3- | |
| | hydroxy-2,6-dimethyl- | |
| | phenyl)carbamoyl]thiazol-2- | |
| | yl]amino]pyrazol-1-yl]acetyl]-2,6- | |
| | dimethyl-piperazine-1-carboxylate | |
| BAA- 157 | 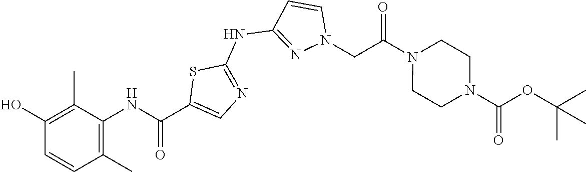 | MS (ES+) m/z 583.9 (M + H). ¹H NMR (300 MHz, Methanol-d₄) δ 8.08 (s, 1H), 7.62 (d, J = 2.4 Hz, 1H), 6.93 (d, J = 8.3 Hz, 1H), 6.70 (d, J = 8.2 Hz, 1H), 6.08 (d, J = 2.4 Hz, 1H), 4.79 (s, 2H), 4.09-3.95 (m, 2H), 3.13 (d, J = 6.5 Hz, 2H), 2.81-2.59 (m, 2H), 2.18 (s, 3H), 2.13 (s, 3H), 1.77-1.67 (m, 3H), 1.44 (s, 9H), 1.13-0.96 (m, 2H). |
| | tert-butyl 4-[[[2-[3-[5-[(3-hydroxy-2,6- | |
| | dimethyl-phenyl)carbamoyl]thiazol-2- | |
| | yl]amino]pyrazol-1- | |
| | yl]acetyl]amino]methyl]piperidine-1- | |
| | carboxylate | |
| BAA- 158 | 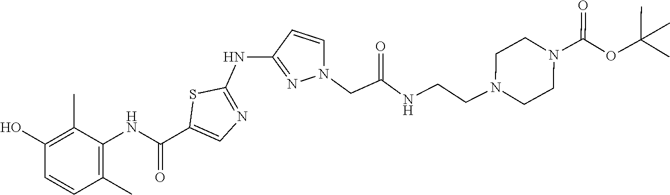 [SMILES: Cc1ccc(O)c(C)c1NC(=O)c1cnc(Nc2ccn(CC(=O)NCCN3CCN(C(=O)OC(C)(C)C)CC3)n2)s1] | MS (ES+) m/z 599.0 (M + H). ¹H NMR (300 MHz, Methanol-d₄) δ 8.09 (s, 1H), 7.66 (d, J = 2.4 Hz, 1H), 6.93 (d, J = 8.2 Hz, 1H), 6.70 (d, J = 8.2 Hz, 1H), 6.12 (d, J = 2.4 Hz, 1H), 4.82 (s, 2H), 3.41-3.34 (m, 2H), 3.31- 3.21 (m, 4H), 2.49 (t, J = 6.2 Hz, 2H), 2.39- 2.29 (m, 4H), 2.18 (s, 3H), 2.13 (s, 3H), 1.45 (s, 9H). |
| | tert-butyl 4-[2-[[2-[3-[[5-[(3-hydroxy- | |
| | 2,6-dimethyl- | |
| | phenyl)carbamoyl]thiazol-2- | |
| | ylJamino]pyrazol-1- | |
| | yl]acetyl]amino]ethyl]piperazine-1- | |
| | carboxylate | |
| BAA- 159 | 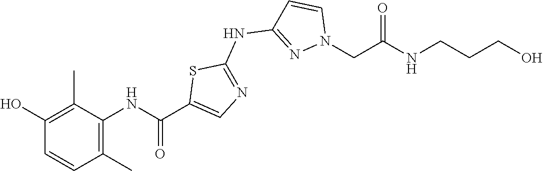 [SMILES: Cc1ccc(O)c(C)c1NC(=O)c1cnc(Nc2ccn(CC(=O)NCCCO)n2)s1] | MS (ES+) m/z 444.9 (M + H). ¹H NMR (300 MHz, Methanol-d₄) δ 8.06 (s, 1H), 7.62 (d, J = 2.4 Hz, 1H), 6.93 (d, J = 8.3 Hz, 1H), 6.70 (d, J = 8.2 Hz, 1H), 6.09 (d, J = 2.4 Hz, 1H), 4.79 (s, 2H), 3.61 (t, J = 6.3 Hz, 2H), 3.38-3.34 (m, 2H), 2.18 (s, 3H), 2.13 (s, 3H), 1.75 (p, J = 6.6 Hz, 2H). |
| | N-(3-hydroxy-2,6-dimethyl-phenyl)-2- | |
| | [[1-[2-(3-hydroxypropylamino)-2-oxo- | |
| | ethyl]pyrazol-3-yl]amino]thiazole-5- | |
| | carboxamide | |
| BAA- 160 | 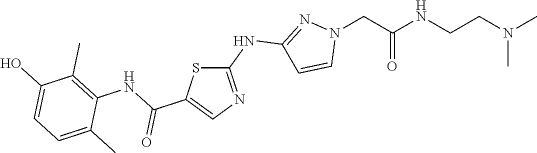 [SMILES: Cc1ccc(O)c(C)c1NC(=O)c1cnc(Nc2ccn(CC(=O)NCCN(C)C)n2)s1] | MS (ES+) m/z 458.0 (M + H). ¹H NMR (300 MHz, DMSO-d₆) δ 11.44-11.04 (br s, 1H), 10.24-10.04 (br s, 1H), 9.60 (s, 1H), 9.42-9.02 (br s, 1H), 8.45 (t, J = 5.7 Hz, 1H), 8.16 (s, 1H), 7.70 (d, J = 2.4 Hz, 1H), 6.88 (d, J = 8.2 Hz, 1H), 6.70 (d, J = 8.2 Hz, 1H), 6.01 (d, J = 2.4 Hz, 1H), 4.79 (s, 2H), 3.47 (q, J = 6.1 Hz, 2H), 3.15 (q, J = 6.0 Hz, 2H), 2.77 (d, J = 4.9 Hz, 6H), 2.07 (s, 3H), 1.98 (s, 3H). ¹H NMR (300 MHz, DMSO-d₆ + D₂O) δ 8.34 (t, |
| | | J = 5.8 Hz, 1H), 8.07 (s, 1H), 7.62 (d, J = 2.4 |
| | 2-[ 1-[2-[2- | Hz, 1H), 6.89 (d, J = 8.3 Hz, 1H), 6.67 (d, J = |
| | (dimethylamino)ethylamino]-2-oxo- | 8.2 Hz, 1H), 6.04 (d, J = 2.4 Hz, 1H), 4.75 (s, |
| | ethyl]pyrazol-3-yl]amino]-N-(3- | 2H), 3.51-3.40 (m, 2H), 3.13 (t, J = 6.0 Hz, |
| | hydroxy-2,6-dimethyl-phenyl)thiazole- | 2H), 2.76 (s, 6H), 2.03 (s, 3H), 1.94 (s, 3H). |
| | 5-carboxamide (hydrochloride) | |
| BAA- 161 | 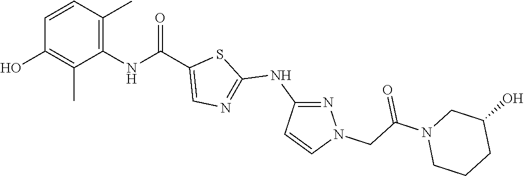 [SMILES: Cc1ccc(O)c(C)c1NC(=O)c1cnc(Nc2ccn(CC(=O)N3CCC[C@@H](O)C3)n2)s1] | MS (ES+) m/z 470.9 (M + H). ¹H NMR (300 MHz, Methanol-d₄) δ 8.05 (d, J = 0.8 Hz, 1H), 7.56 (dd, J = 2.4, 0.9 Hz, 1H), 6.93 (d, J = 8.2 Hz, 1H), 6.70 (d, J = 8.2 Hz, 1H), 6.09 (dd, J = 2.4, 1.2 Hz, 1H), 5.21-4.97 (m, 2H), 4.10 (dd, J = 12.7, 3.9 Hz, 0.5H), 3.88- 3.35 (m, 4H), 3.00 (dd, J = 12.7, 8.4 Hz, 0.5H), 2.17 (s, 3H), 2.13 (s, 3H), 2.04-1.75 (m, 2H), 1.70-1.41 (m, 2H). |
| | N-(3-hydroxy-2,6-dimethyl-phenyl)-2- | |
| | [1-[2-[(3R)-3-hydroxy-1-piperidyl]-2- | |
| | oxo-ethyl]pyrazol-3-yl]amino]thiazole- | |
| | 5-carboxamide | |
| BAA- 162 | 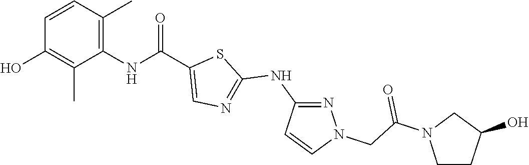 [SMILES: Cc1ccc(O)c(C)c1NC(=O)c1cnc(Nc2ccn(CC(=O)N3CC[C@H](O)C3)n2)s1] | MS (ES+) m/z 456.9 (M + H). ¹H NMR (300 MHz, Methanol-d₄) δ 8.05 (s, 1H), 7.59 (d, J = 2.4 Hz, 1H), 6.93 (d, J = 8.3 Hz, 1H), 6.70 (d, J = 8.2 Hz, 1H), 6.09 (dd, J = 2.4, 0.9 Hz, 1H), 5.13-4.94 (m, 2H), 4.57-4.41 (m, 1H), 3.94-3.51 (m, 4H), 2.18 (s, 3H), 2.13 (s, 3H), 2.12-1.88 (m, 2H). |
| | N-(3-hydroxy-2,6-dimethyl-phenyl)-2- | |
| | [1-[2-[(3S)-3-hydroxypyrrolidin-1-yl]- | |
| | 2-oxo-ethyl]pyrazol-3- | |
| | ylamino]thiazole-5-carboxamide | |
| BAA- 163 | 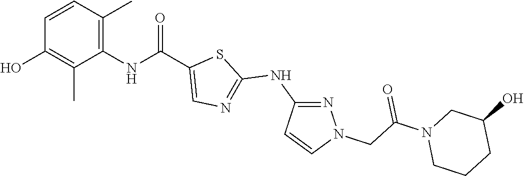 [SMILES: Cc1ccc(O)c(C)c1NC(=O)c1cnc(Nc2ccn(CC(=O)N3CCC[C@H](O)C3)n2)s1] | MS (ES+) m/z 470.9 (M + H). ¹H NMR (300 MHz, Methanol-d₄) δ 8.05 (d, J = 0.9 Hz, 1H), 7.56 (dd, J = 2.4, 0.8 Hz, 1H), 6.93 (d, J = 8.2 Hz, 1H), 6.70 (d, J = 8.2 Hz, 1H), 6.09 (dd, J = 2.5, 1.1 Hz, 1H), 5.23-4.96 (m, 2H), 4.11 (dd, J = 12.7, 3.9 Hz, 0.5H), 3.89- 3.34 (m, 4H), 3.00 (dd, J = 12.7, 8.4 Hz, 0.5H), 2.18 (s, 3H), 2.13 (s, 3H), 2.04-1.74 (m, 2H), 1.71-1.41 (m, 2H). |
| | N-(3-hydroxy-2,6-dimethyl-phenyl)-2- | |
| | [1-[2-[(3S)-3-hydroxy-1-piperidyl]-2- | |
| | oxo-ethyl]pyrazol-3-yl]amino]thiazole- | |
| | 5-carboxamide | |
| BAA- 164 | 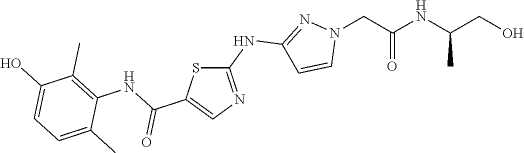 [SMILES: Cc1ccc(O)c(C)c1NC(=O)c1cnc(Nc2ccn(CC(=O)N[C@H](C)CO)n2)s1] | MS (ES+) m/z 444.9 (M + H). ¹H NMR (300 MHz, Methanol-d₄) δ 8.06 (s, 1H), 7.62 (d, J = 2.4 Hz, 1H), 6.93 (d, J = 8.3 Hz, 1H), 6.70 (d, J = 8.2 Hz, 1H), 6.10 (d, J = 2.4 Hz, 1H), 4.80 (s, 2H), 4.06-3.94 (m, 1H), 3.52 (d, J = 5.4 Hz, 2H), 2.18 (s, 3H), 2.13 (s, 3H), 1.18 (d, J = 6.8 Hz, 3H). |
| | N-(3-hydroxy-2,6-dimethyl-phenyl)-2- | |
| | [1-[2-[(1R)-2-hydroxy-1-methyl- | |
| | ethyl]amino]-2-oxo-ethyl]pyrazol-3- | |
| | yl]amino]thiazole-5-carboxamide | |
| BAA- 165 | 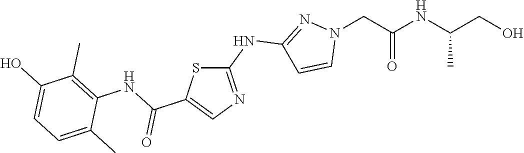 [SMILES: Cc1ccc(O)c(C)c1NC(=O)c1cnc(Nc2ccn(CC(=O)N[C@@H](C)CO)n2)s1] | MS (ES+) m/z 444.9 (M + H). ¹H NMR (300 MHz, Methanol-d₄) δ 8.06 (s, 1H), 7.62 (d, J = 2.4 Hz, 1H), 6.93 (d, J = 8.3 Hz, 1H), 6.70 (d, J = 8.2 Hz, 1H), 6.10 (d, J = 2.4 Hz, 1H), 4.80 (s, 2H), 4.06-3.94 (m, 1H), 3.52 (d, J = 5.4 Hz, 2H), 2.18 (s, 3H), 2.13 (s, 3H), 1.18 (d, J = 6.8 Hz, 3H). |
| | N-(3-hydroxy-2,6-dimethyl-phenyl)-2- | |
| | [1-[2-[[(1S)-2-hydroxy-1-methyl- | |
| | ethyl]amino]-2-oxo-ethyl]pyrazol-3- | |
| | yl]amino]thiazole-5-carboxamide | |
| BAA- 166 | 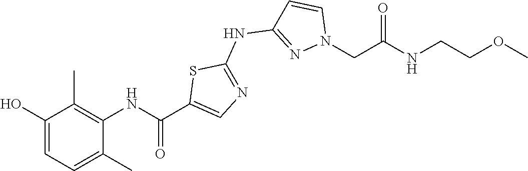 [SMILES: COCCNC(=O)Cn1ccc(Nc2ncc(C(=O)Nc3c(C)ccc(O)c3C)s2)n1] | MS (ES+) m/z 444.9 (M + H). ¹H NMR (300 MHz, Methanol-d₄) δ 8.06 (s, 1H), 7.63 (d, J = 2.4 Hz, 1H), 6.93 (d, J = 8.2 Hz, 1H), 6.70 (d, J = 8.2 Hz, 1H), 6.10 (d, J = 2.4 Hz, 1H), 4.82 (s, 2H), 3.50-3.39 (m, 4H), 3.32 (s, 3H), 2.18 (s, 3H), 2.13 (s, 3H). |
| | N-(3-hydroxy-2,6-dimethyl-phenyl)-2- | |
| | [[1-[2-(2-methoxyethylamino)-2-oxo- | |
| | ethyl]pyrazol-3-yl]amino]thiazole-5- | |
| | carboxamide | |
| BAA- 167 | 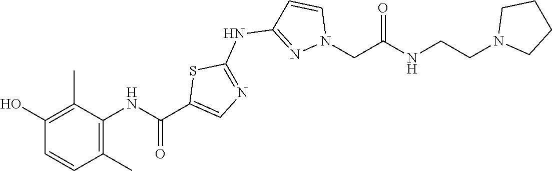 [SMILES: Cc1ccc(O)c(C)c1NC(=O)c1cnc(Nc2ccn(CC(=O)NCCN3CCCC3)n2)s1] | MS (ES+) m/z 484.0 (M + H). ¹H NMR (300 MHz, DMSO-d₆) δ 11.47-11.07 (br s, 1H), 10.57-10.37 (br s, 1H), 9.61 (s, 1H), 9.35-8.95 (br s, 1H), 8.46 (t, J = 5.7 Hz, 1H), 8.17 (s, 1H), 7.71 (d, J = 2.4 Hz, 1H), 6.88 (d, J = 8.2 Hz, 1H), 6.70 (d, J = 8.1 Hz, 1H), 6.00 (d, J = 2.3 Hz, 1H), 4.80 (s, 2H), 3.61- 3.51 (m, 2H), 3.47 (q, J = 6.2 Hz, 2H), 3.22 (q, J = 6.1 Hz, 2H), 3.05-2.89 (m, 2H), 2.07 (s, 3H), 1.98 (s, 3H), 1.97-1.78 (m, 4H). ¹H NMR (300 MHz, DMSO-d₆ + D₂O) δ 8.37 (t, |
| | | J = 5.8 Hz, 1H), 8.09 (s, 1H), 7.65 (d, J = 2.4 |
| | N-(3-hydroxy-2,6-dimethyl-phenyl)-2- | Hz, 1H), 6.89 (d, J = 8.3 Hz, 1H), 6.67 (d, J = |
| | [1-[2-oxo-2-(2-pyrrolidin-1- | 8.2 Hz, 1H), 6.03 (d, J = 2.4 Hz, 1H), 4.75 (s, |
| | ylethylamino)ethyl]pyrazol-3- | 2H), 3.57-3.48 (m, 2H), 3.48-3.40 (m, 2H), |
| | yl]amino]thiazole-5-carboxamide | 3.21 (t, J = 6.1 Hz, 2H), 3.04-2.90 (m, 2H), |
| | (hydrochloride) | 2.04 (s, 3H), 2.00-1.71 (m, 7H). |
| BAA- 168 | 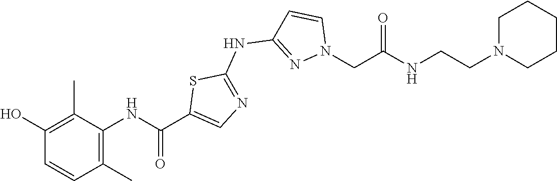 [SMILES: Cc1ccc(O)c(C)c1NC(=O)c1cnc(Nc2ccn(CC(=O)NCCN3CCCCC3)n2)s1] | MS (ES+) m/z 498.0 (M + H). ¹H NMR (300 MHz, DMSO-d₆) δ 11.53-11.13 (br s, 1H), 10.24-10.04 (br s, 1H), 9.63 (s, 1H), 9.35-8.95 (br s, 1H), 8.51 (t, J = 5.7 Hz, 1H), 8.18 (s, 1H), 7.71 (d, J = 2.4 Hz, 1H), 6.88 (d, J = 8.2 Hz, 1H), 6.70 (d, J = 8.2 Hz, 1H), 6.00 (d, J = 2.3 Hz, 1H), 4.78 (s, 2H), 3.51 (q, J = 6.1 Hz, 2H), 3.46-3.38 (m, 2H), 3.11 (q, J = 6.2 Hz, 2H), 2.95-2.76 (m, 2H), 2.07 (s, 3H), 1.98 (s, 3H), 1.81-1.60 (m, 5H), 1.38-1.24 (m, 1H). |
| | | ¹H NMR (300 MHz, DMSO-d₆ + D₂O) δ 8.09 (s, |
| | N-(3-hydroxy-2,6-dimethyl-phenyl)-2- | 1H), 7.64 (d, J = 2.4 Hz, 1H), 6.89 (d, J = 8.3 |
| | [[1-[2-oxo-2-[2-(1- | Hz, 1H), 6.67 (d, J = 8.2 Hz, 1H), 6.03 (d, J = |
| | piperidyl)ethylamino]ethyl]pyrazol-3- | 2.4 Hz, 1H), 4.74 (s, 2H), 3.51-3.35 (m, 4H), |
| | yl]amino]thiazole-5-carboxamide | 3.10 (t, J = 6.3 Hz, 2H), 2.83 (td, J = 12.2, 3.1 |
| | (hydrochloride) | Hz, 2H), 2.03 (s, 3H), 1.95 (s, 3H), 1.79-1.54 |
| | | (m, 5H), 1.37-1.24 (m, 1H). |
| BAA- 169 | 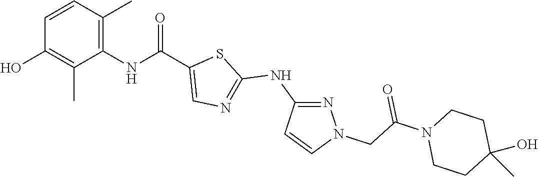 [SMILES: Cc1ccc(O)c(C)c1NC(=O)c1cnc(Nc2ccn(CC(=O)N3CCC(C)(O)CC3)n2)s1] | MS (ES+) m/z 485.0 (M + H). ¹H NMR (300 MHz, DMSO-d₆) δ 11.04 (s, 1H), 9.51 (s, 1H), 9.19 (s, 1H), 8.10 (s, 1H), 7.59 (d, J = 2.4 Hz, 1H), 6.88 (d, J = 8.2 Hz, 1H), 6.68 (d, J = 8.2 Hz, 1H), 5.98 (d, J = 2.3 Hz, 1H), 5.17-4.91 (m, 2H), 4.55-4.15 (br s, 1H), 3.98- 3.85 (m, 1H), 3.69-3.57 (m, 1H), 3.12-3.00 (m, 1H), 2.07 (s, 3H), 1.98 (s, 3H), 1.56-1.27 (m, 4H), 1.13 (s, 3H). ¹H NMR (300 MHz, DMSO-d₆ + D₂O) δ 8.04 (s, 1H), 7.55 (d, J = 2.4 Hz, 1H), 6.88 (d, J = 8.3 |
| | | Hz, 1H), 6.66 (d, J = 8.2 Hz, 1H), 6.02 (d, J = |
| | N-(3-hydroxy-2,6-dimethyl-phenyl)-2- | 2.4 Hz, 1H), 5.11-4.87 (m, 2H), 3.91-3.80 |
| | [[1-[2-(4-hydroxy-4-methyl-1- | (m, 1H), 3.62-3.51 (m, 1H), 3.44-3.31 (m, |
| | piperidyl)-2-oxo-ethyl]pyrazol-3- | 1H), 3.13-3.01 (m, 1H), 2.03 (s, 3H), 1.94 (s, |
| | yl]amino]thiazole-5-carboxamide | 3H), 1.53-1.30 (m, 4H), 1.10 (s, 3H). |
| BAA- 170 | 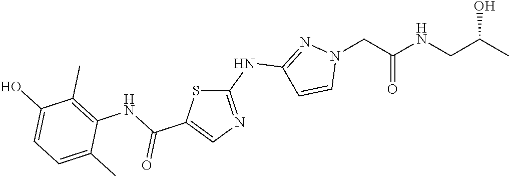 [SMILES: Cc1ccc(O)c(C)c1NC(=O)c1cnc(Nc2ccn(CC(=O)NC[C@@H](C)O)n2)s1] | MS (ES+) m/z 444.9 (M + H). ¹H NMR (300 MHz, Methanol-d₄) δ 8.06 (s, 1H), 7.64 (d, J = 2.4 Hz, 1H), 6.93 (d, J = 8.3 Hz, 1H), 6.70 (d, J = 8.2 Hz, 1H), 6.11 (d, J = 2.4 Hz, 1H), 4.83 (s, 2H), 3.93-3.80 (m, 1H), 3.31- 3.11 (m, 2H), 2.18 (s, 3H), 2.13 (s, 3H), 1.15 (d, J = 6.3 Hz, 3H). |
| | N-(3-hydroxy-2,6-dimethyl-phenyl)-2- | |
| | [[1-[2-[[(2R)-2-hydroxypropyl]amino]- | |
| | 2-oxo-ethyl]pyrazol-3- | |
| | yl]amino]thiazole-5-carboxamide | |
| BAA- 171 | 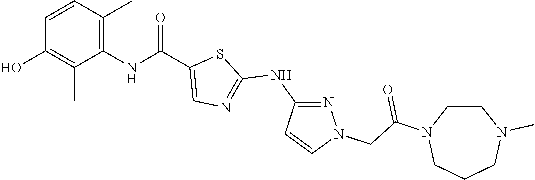 [SMILES: Cc1ccc(O)c(C)c1NC(=O)c1cnc(Nc2ccn(CC(=O)N3CCCN(C)CC3)n2)s1] | MS (ES+) m/z 483.9 (M + H). ¹H NMR (300 MHz, DMSO-d₆) δ 11.43-11.03 (br s, 1H), 10.82-10.62 (br s, 1H), 9.60 (s, 1H), 9.41-9.01 (br s, 1H), 8.16 (s, 1H), 7.62 (t, J = 2.3 Hz, 1H), 6.88 (d, J = 8.2 Hz, 1H), 6.70 (d, J = 8.2 Hz, 1H), 6.01 (t, J = 1.7 Hz, 1H), 5.23-5.01 (m, 2H), 4.10-3.95 (m, 1H), 3.84- 3.58 (m, 2H), 3.58-3.40 (m, 3H), 3.35-3.21 (m, 1H), 3.14-2.93 (m, 1H), 2.88-2.72 (m, 3H), 2.39-2.08 (m, 2H), 2.07 (s, 3H), 1.98 (s, 3H) |
| | | ¹H NMR (300 MHz, DMSO-d₆ + D₂O) δ 8.09 (s, |
| | N-(3-hydroxy-2,6-dimethyl-phenyl)-2- | 1H), 7.58 (d, J = 2.4 Hz, 1H), 6.88 (d, J = 8.2 |
| | [1-[2-(4-methyl-1,4-diazepan-1-yl)-2- | Hz, 1H), 6.67 (d, J = 8.2 Hz, 1H), 6.04 (d, J = |
| | oxo-ethyl]pyrazol-3-yl]amino]thiazole- | 2.4 Hz, 1H), 5.20-4.90 (m, 2H), 3.70-3.38 |
| | 5-carboxamide (hydrochloride) | (m, 5H), 3.32-3.17 (m, 1H), 3.03 (s, 1H), 2.80 |
| | | (d, J = 13.5 Hz, 3H), 2.13 (s, 2H), 2.04 (s, 3H), |
| | | 1.95 (s, 3H). |
| BAA- 172 | 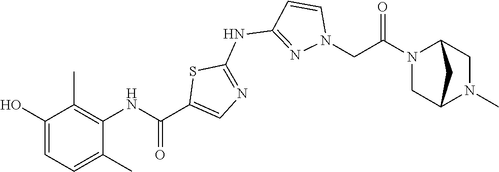 [SMILES: Cc1ccc(O)c(C)c1NC(=O)c1cnc(Nc2ccn(CC(=O)N3C[C@@H]4C[C@H]3CN4C)n2)s1] | MS (ES+) m/z 481.9 (M + H). ¹H NMR (300 MHz, DMSO-d₆) δ 11.76-10.44 (m, 2H), 9.62 (s, 1H), 9.18 (s, 1H), 8.17 (d, J = 1.5 Hz, 1H), 7.68-7.59 (m, 1H), 6.88 (d, J = 8.2 Hz, 1H), 6.70 (d, J = 8.1 Hz, 1H), 6.11- 5.90 (m, 1H), 5.20-4.89 (m, 3H), 4.48-4.29 (m, 1H), 4.12-3.42 (m, 3H), 3.17-2.93 (m, 1H), 2.92-2.77 (m, 3H), 2.44-2.19 (m, 1H), 2.21-2.02 (m, 4H), 1.98 (s, 3H). ¹H NMR (300 MHz, DMSO-d₆ + D₂O) δ 8.11 (s, 1H), 7.72-7.50 (m, 1H), 6.88 (d, J = 8.3 Hz, |
| | | 1H), 6.68 (d, J = 8.2 Hz, 1H), 6.09-5.95 (m, |
| | N-(3-hydroxy-2,6-dimethyl-phenyl)-2- | 1H), 5.17-4.69 (m, 3H), 4.48-4.18 (m, 1H), |
| | [1-[2-[(1S,4S)-5-methyl-2,5- | 3.74-3.42 (m, 3H), 3.21-3.01 (m, 1H), 2.94- |
| | diazabicyclo[2.2. 1 ]heptan-2-yl]-2-oxo- | 2.78 (m, 3H), 2.39-2.07 (m, 2H), 2.04 (s, 3H), |
| | ethyl]pyrazol-3-yl]amino]thiazole-5- | 1.96 (s, 3H). |
| | carboxamide (hydrochloride) | |
| BAA- 173 | 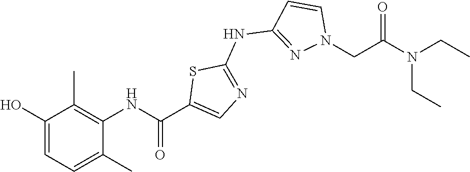 [SMILES: CCN(CC)C(=O)Cn1ccc(Nc2ncc(C(=O)Nc3c(C)ccc(O)c3C)s2)n1] | MS (ES+) m/z 442.9 (M + H). ¹H NMR (300 MHz, Methanol-d₄) δ 8.05 (s, 1H), 7.58 (d, J = 2.4 Hz, 1H), 6.93 (d, J = 8.2 Hz, 1H), 6.70 (d, J = 8.2 Hz, 1H), 6.09 (d, J = 2.4 Hz, 1H), 5.05 (s, 2H), 3.56 (q, J = 7.1 Hz, 2H), 3.43 (q, J = 7.1 Hz, 2H), 2.18 (s, 3H), 2.13 (s, 3H), 1.29 (t, J = 7.1 Hz, 3H), 1.16 (t, J = 7.1 Hz, 3H) |
| | 2-[[1-[2-(diethylamino)-2-oxo- | |
| | ethyl]pyrazol-3-yl]amino]-N-(3- | |
| | hydroxy-2,6-dimethyl-phenyl)thiazole- | |
| | 5-carboxamide | |
| BAA- 174 | 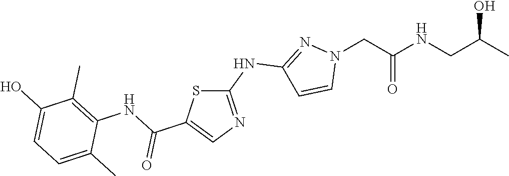 [SMILES: Cc1ccc(O)c(C)c1NC(=O)c1cnc(Nc2ccn(CC(=O)NC[C@H](C)O)n2)s1] | MS (ES+) m/z 444.9 (M + H). ¹H NMR (300 MHz, Methanol-d₄) δ 8.06 (s, 1H), 7.64 (d, J = 2.4 Hz, 1H), 6.93 (d, J = 8.3 Hz, 1H), 6.70 (d, J = 8.2 Hz, 1H), 6.11 (d, J = 2.4 Hz, 1H), 4.83 (s, 2H), 3.92-3.80 (m, 1H), 3.31- 3.12 (m, 2H), 2.18 (s, 3H), 2.13 (s, 3H), 1.15 (d, J = 6.3 Hz, 3H). |
| | N-(3-hydroxy-2,6-dimethyl-phenyl)-2- | |
| | [1-[2-[(2S)-2-hydroxypropyl]amino]- | |
| | 2-oxo-ethyl]pyrazol-3- | |
| | yl]amino]thiazole-5-carboxamide | |
| BAA- 175 | 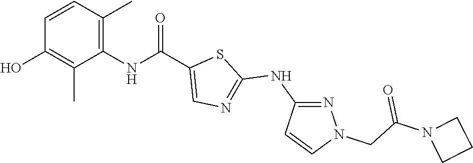 [SMILES: Cc1ccc(O)c(C)c1NC(=O)c1cnc(Nc2ccn(CC(=O)N3CCC3)n2)s1] | MS (ES+) m/z 426.9 (M + H). ¹H NMR (300 MHz, DMSO-d⁶) δ 11.09 (s, 1H), 9.52 (s, 1H), 9.19 (s, 1H), 8.11 (s, 1H), 7.63 (d, J = 2.4 Hz, 1H), 6.89 (d, J = 8.2 Hz, 1H), 6.69 (d, J = 8.2 Hz, 1H), 5.99 (d, J = 2.3 Hz, 1H), 4.75 (s, 2H), 4.17 (t, J = 7.6 Hz, 2H), 3.90 (t, J = 7.7 Hz, 2H), 2.23 (p, J = 7.6 Hz, 2H), 2.07 (s, 3H), 1.99 (s, 3H). ¹H NMR (300 MHz, DMSO-d₆ + D₂O) δ 8.08 (s, 1H), 7.61 (d, J = 2.4 Hz, 1H), 6.89 (d, J = 8.2 |
| | | Hz, 1H), 6.68 (d, J = 8.2 Hz, 1H), 6.00 (d, J = |
| | 2-[[1-[2-(azetidin-1-yl)-2-oxo- | 2.4 Hz, 1H), 4.71 (s, 2H), 4.16 (t, J = 7.7 Hz, |
| | ethyl]pyrazol-3-yl]amino]-N-(3- | 2H), 3.89 (t, J = 7.8 Hz, 2H), 2.22 (p, J = 7.6 |
| | hydroxy-2,6-dimethyl-phenyl)thiazole- | Hz, 2H), 2.05 (s, 3H), 1.97 (s, 3H). |
| | 5-carboxamide | |
| BAA- 176 | 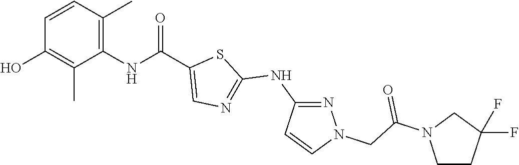 [SMILES: Cc1ccc(O)c(C)c1NC(=O)c1cnc(Nc2ccn(CC(=O)N3CCC(F)(F)C3)n2)s1] | MS (ES+) m/z 476.9 (M + H). ¹H NMR (300 MHz, Methanol-d₄) δ 8.06 (d, J = 1.8 Hz, 1H), 7.58 (t, J = 2.4 Hz, 1H), 6.93 (d, J = 8.3 Hz, 1H), 6.70 (d, J = 8.2 Hz, 1H), 6.10 (dd, J = 3.9, 2.4 Hz, 1H), 5.01 (d, J = 11.6 Hz, 2H), 4.10-3.93 (m, 2H), 3.81 (t, J = 12.9 Hz, 1H), 3.72 (t, J = 7.6 Hz, 1H), 2.64-2.51 (m, 1H), 2.50-2.36 (m, 1H), 2.18 (s, 3H), 2.13 (s, 3H). ¹⁹F NMR (282 MHz, Methanol-d₄) δ −103.05, |
| | | −104.11. |
| | 2-[1-[2-(3,3-difluoropyrrolidin-1-yl)-2- | |
| | oxo-ethyl]pyrazol-3-yl]amino]-N-(3- | |
| | hydroxy-2,6-dimethyl-phenyl)thiazole- | |
| | 5-carboxamide | |
| BAA- 177 | 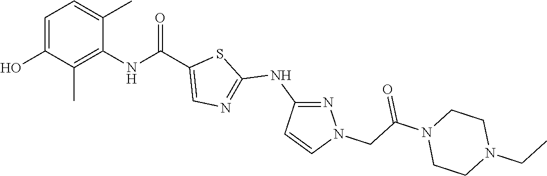 [SMILES: CCN1CCN(C(=O)Cn2ccc(Nc3ncc(C(=O)Nc4c(C)ccc(O)c4C)s3)n2)CC1] | MS (ES+) m/z 484.0 (M + H). ¹H NMR (300 MHz, DMSO-d₆) δ 11.04 (s, 1H), 9.51 (s, 1H), 9.19 (s, 1H), 8.10 (s, 1H), 7.60 (d, J = 2.4 Hz, 1H), 6.88 (d, J = 8.2 Hz, 1H), 6.68 (d, J = 8.2 Hz, 1H), 5.98 (d, J = 2.3 Hz, 1H), 5.06 (s, 2H), 3.56-3.40 (m, 4H), 2.44-2.28 (m, 6H), 2.07 (s, 3H), 1.98 (s, 3H), 1.00 (t, J = 7.1 Hz, 3H). ¹H NMR (300 MHz, DMSO-d₆ + D₂O) δ 8.06 (s, 1H), 7.57 (d, J = 2.4 Hz, 1H), 6.88 (d, J = 8.3 Hz, 1H), 6.67 (d, J = 8.2 Hz, 1H), 6.01 (d, J = |
| | | 2.4 Hz, 1H), 5.01 (s, 2H), 3.55-3.39 (m, 4H), |
| | 2-[1-[2-(4-ethylpiperazin-1-yl)-2-oxo- | 2.42-2.25 (m, 6H), 2.04 (s, 3H), 1.95 (s, 3H), |
| | ethyl]pyrazol-3-yl]amino]-N-(3- | 0.96 (t, J = 7.2 Hz, 3H). |
| | hydroxy-2,6-dimethyl-phenyl)thiazole- | |
| | 5-carboxamide | |
| BAA- 178 | 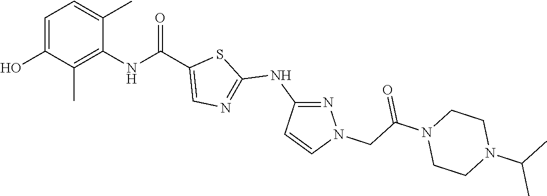 [SMILES: Cc1ccc(O)c(C)c1NC(=O)c1cnc(Nc2ccn(CC(=O)N3CCN(C(C)C)CC3)n2)s1] | MS (ES+) m/z 498.0 (M + H). ¹H NMR (300 MHz, DMSO-d₆) δ 11.04 (s, 1H), 9.50 (s, 1H), 9.18 (s, 1H), 8.10 (s, 1H), 7.60 (d, J = 2.4 Hz, 1H), 6.88 (d, J = 8.2 Hz, 1H), 6.68 (d, J = 8.2 Hz, 1H), 5.98 (d, J = 2.3 Hz, 1H), 5.05 (s, 2H), 3.55-3.39 (m, 4H), 2.74-2.63 (m, 1H), 2.49-2.34 (m, 4H), 2.07 (s, 3H), 1.98 (s, 3H), 0.96 (d, J = 6.5 Hz, 6H). ¹H NMR (300 MHz, DMSO-d₆ + D₂O) δ 8.05 (s, 1H), 7.57 (d, J = 2.4 Hz, 1H), 6.88 (d, J = 8.3 Hz, 1H), 6.67 (d, J = 8.2 Hz, 1H), 6.01 (d, J = 2.4 Hz, 1H), 4.99 (s, 2H), 3.55-3.39 (m, 4H), |
| | | 2.60 (p, J = 6.5 Hz, 1H), 2.48-2.34 (m, 4H), |
| | N-(3-hydroxy-2,6-dimethyl-phenyl)-2- | 2.03 (s, 3H), 1.95 (s, 3H), 0.92 (d, J = 6.4 Hz, |
| | [ 1-[2-(4-isopropylpiperazin-1-yl)-2- | 6H). |
| | oxo-ethyl]pyrazol-3-yl]amino]thiazole- | |
| | 5-carboxamide | |
| BAA- 179 | 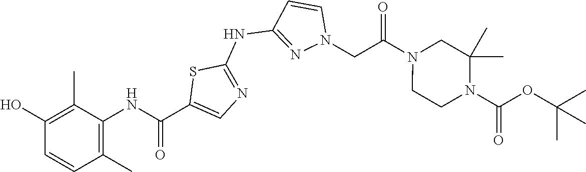 [SMILES: Cc1ccc(O)c(C)c1NC(=O)c1cnc(Nc2ccn(CC(=O)N3CCN(C(=O)OC(C)(C)C)C(C)(C)C3)n2)s1] | MS (ES+) m/z 583.9 (M + H). ¹H NMR (300 MHz, Methanol-d₄) δ 8.05 (d, J = 1.8 Hz, 1H), 7.59 (dd, J = 3.1, 2.5 Hz, 1H), 6.93 (d, J = 8.2 Hz, 1H), 6.70 (d, J = 8.2 Hz, 1H), 6.10 (dd, J = 2.5, 1.7 Hz, 1H), 5.07 (d, J = 6.2 Hz, 2H), 3.81 (s, 2H), 3.78-3.72 (m, 1H), 3.70 (s, 1H), 3.65 (s, 1H), 3.57-3.50 (m, 1H), 2.18 (s, 3H), 2.13 (s, 3H), 1.48 (d, J = 3.1 Hz, 9H), 1.45 (s, 3H), 1.41 (s, 3H). |
| | tert-butyl 4-[2-[3-[[5-[(3-hydroxy-2,6- | |
| | dimethyl-phenyl)carbamoyl]thiazol-2- | |
| | yl]amino]pyrazol-1-yl]acetyl]-2,2- | |
| | dimethyl-piperazine-1-carboxylate | |
| BAA- 180 | 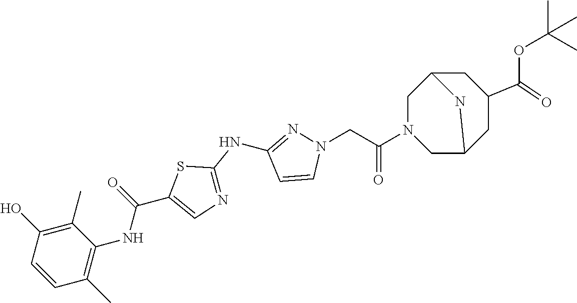 | MS (ES+) m/z 581.9 (M + H). ¹H NMR (300 MHz, Methanol-d₄) δ 8.05 (s, 1H), 7.58 (d, J = 2.4 Hz, 1H), 6.93 (d, J = 8.2 Hz, 1H), 6.70 (d, J = 8.2 Hz, 1H), 6.11 (d, J = 2.4 Hz, 1H), 5.21 (d, J = 16.3 Hz, 1H), 4.97 (s, 1H), 4.27 (s, 2H), 4.23-4.16 (m, 1H), 3.93-3.76 (m, 1H), 3.41 (dd, J = 12.6, 2.0 Hz, 1H), 2.94 (d, J = 13.0 Hz, 1H), 2.18 (s, 3H), 2.13 (s, 3H), 1.98-1.87 (m, 2H), 1.84-1.61 (m, 2H), 1.48 (s, 9H). |
| | tert-butyl 3-[2-[3-[5-[(3-hydroxy-2,6- | |
| | dimethyl-phenyl)carbamoyl]thiazol-2- | |
| | yl]amino]pyrazol-1-yl]acetyl]-3,8- | |
| | diazabicyclo[3.2. 1]octane-8- | |
| | carboxylate | |
| BAA- 181 | 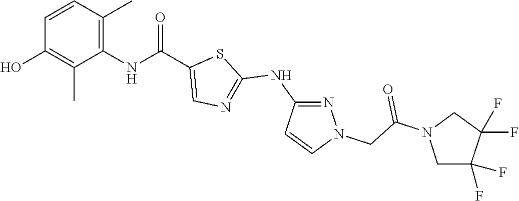 [SMILES: Cc1ccc(O)c(C)c1NC(=O)c1cnc(Nc2ccn(CC(=O)N3CC(F)(F)C(F)(F)C3)n2)s1] | MS (ES+) m/z 512.9 (M + H). ¹H NMR (300 MHz, Methanol-d₄) δ 8.06 (s, 1H), 7.59 (d, J = 2.5 Hz, 1H), 6.93 (d, J = 8.2 Hz, 1H), 6.70 (d, J = 8.2 Hz, 1H), 6.12 (d, J = 2.5 Hz, 1H), 5.07 (s, 2H), 4.35 (t, J = 13.4 Hz, 2H), 4.05 (t, J = 13.8 Hz, 2H), 2.18 (s, 3H), 2.13 (s, 3H). ¹⁹F NMR (282 MHz, Methanol-d₄) δ −124.96, −124.98, −125.00, −126.02, −126.04, −126.06. |
| | N-(3-hydroxy-2,6-dimethyl-phenyl)-2- | |
| | [[1-[2-oxo-2-(3,3,4,4- | |
| | tetrafluoropyrrolidin-1- | |
| | yl)ethyl]pyrazol-3-yl]amino]thiazole-5- | |
| | carboxamide | |
| BAA- 182 | 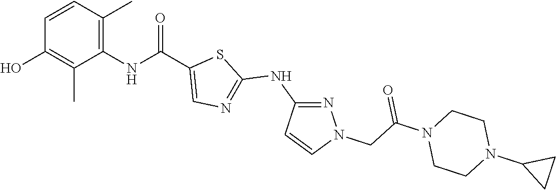 [SMILES: Cc1ccc(O)c(C)c1NC(=O)c1cnc(Nc2ccn(CC(=O)N3CCN(C4CC4)CC3)n2)s1] | MS (ES+) m/z 496.0 (M + H). ¹H NMR (300 MHz, DMSO-d₆) δ 11.05 (s, 1H), 9.51 (s, 1H), 9.19 (s, 1H), 8.10 (s, 1H), 7.60 (d, J = 2.4 Hz, 1H), 6.88 (d, J = 8.2 Hz, 1H), 6.68 (d, J = 8.2 Hz, 1H), 5.98 (d, J = 2.3 Hz, 1H), 5.07 (s, 2H), 3.59-3.39 (m, 4H), 2.57 (s, 4H), 2.07 (s, 3H), 1.99 (s, 3H), 1.64 (s, 1H), 0.43 (s, 2H), 0.34 (s, 2H). ¹H NMR (300 MHz, DMSO-d₆ + D₂O) δ 8.07 (s, 1H), 7.57 (d, J = 2.4 Hz, 1H), 6.88 (d, J = 8.3 Hz, 1H), 6.67 (d, J = 8.2 Hz, 1H), 6.01 (d, J = 2.4 Hz, 1H), 5.03 (s, 2H), 3.52-3.37 (m, 4H), |
| | | 2.58 (s, 4H), 2.04 (s, 3H), 1.96 (s, 3H), 1.67 (s, |
| | 2-[[1-[2-(4-cyclopropylpiperazin-1-yl)- | 1H), 0.46-0.39 (m, 2H), 0.35 (s, 2H). |
| | 2-oxo-ethyl]pyrazol-3-yl]amino]-N-(3- | |
| | hydroxy-2,6-dimethyl-phenyl)thiazole- | |
| | 5-carboxamide | |
| BAA- 183 | 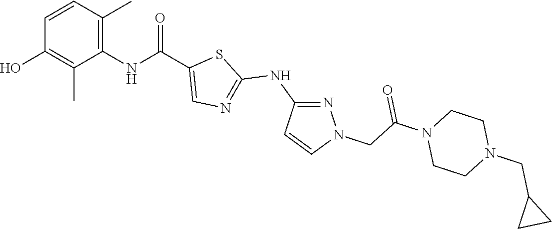 [SMILES: Cc1ccc(O)c(C)c1NC(=O)c1cnc(Nc2ccn(CC(=O)N3CCN(CC4CC4)CC3)n2)s1] | MS (ES+) m/z 510.0 (M + H). ¹H NMR (300 MHz, DMSO-d₆) δ 11.05 (s, 1H), 9.51 (s, 1H), 9.19 (s, 1H), 8.10 (s, 1H), 7.60 (d, J = 2.3 Hz, 1H), 6.88 (d, J = 8.2 Hz, 1H), 6.68 (d, J = 8.2 Hz, 1H), 5.98 (d, J = 2.3 Hz, 1H), 5.06 (s, 2H), 3.70-3.39 (m, 4H), 2.44 (s, 4H), 2.21 (s, 2H), 2.07 (s, 3H), 1.99 (s, 3H), 0.84 (s, 1H), 0.47 (d, J = 7.8 Hz, 2H), 0.08 (s, 2H). ¹H NMR (300 MHz, DMSO-d₆ + D₂O) δ 8.06 (s, 1H), 7.57 (d, J = 2.4 Hz, 1H), 6.88 (d, J = 8.3 Hz, 1H), 6.67 (d, J = 8.2 Hz, 1H), 6.01 (d, J = 2.4 Hz, 1H), 5.01 (s, 2H), 3.62-3.42 (m, 4H), 2.61-2.42 (m, 4H), 2.23 (d, J = 6.7 Hz, 2H), 2.04 (s, 3H), 1.95 (s, 3H), 0.88-0.73 (m, 1H), |
| | | 0.50-0.37 (m, 2H), 0.06 (t, J = 5.1 Hz, 2H). |
| | 2-[ 1-[2-[4- | |
| | (cyclopropylmethyl)piperazin-1-yl]-2- | |
| | oxo-ethyl]pyrazol-3-yl]amino]-N-(3- | |
| | hydroxy-2,6-dimethyl-phenyl)thiazole- | |
| | 5-carboxamide | |
| BAA- 184 | 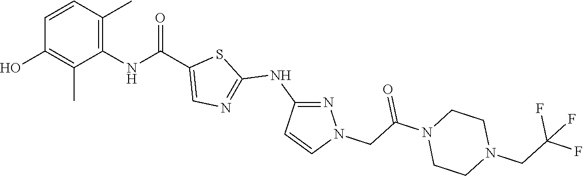 [SMILES: Cc1ccc(O)c(C)c1NC(=O)c1cnc(Nc2ccn(CC(=O)N3CCN(CC(F)(F)F)CC3)n2)s1] | MS (ES+) m/z 538.0 (M + H). ¹H NMR (300 MHz, DMSO-d₆) δ 11.05 (s, 1H), 9.51 (s, 1H), 9.18 (s, 1H), 8.10 (s, 1H), 7.60 (d, J = 2.4 Hz, 1H), 6.88 (d, J = 8.2 Hz, 1H), 6.68 (d, J = 8.2 Hz, 1H), 5.98 (d, J = 2.4 Hz, 1H), 5.07 (s, 2H), 3.58-3.43 (m, 4H), 3.23 (q, J = 10.4 Hz, 2H), 2.71-2.56 (m, 4H), 2.07 (s, 3H), 1.98 (s, 3H). ¹H NMR (300 MHz, DMSO-d₆ + D₂O) δ 8.06 (s, 1H), 7.57 (d, J = 2.4 Hz, 1H), 6.88 (d, J = 8.3 Hz, 1H), 6.67 (d, J = 8.2 Hz, 1H), 6.01 (d, J = |
| | | 2.4 Hz, 1H), 5.02 (s, 2H), 3.55-3.40 (m, 4H), |
| | N-(3-hydroxy-2,6-dimethyl-phenyl)-2- | 3.17 (q, J = 10.1 Hz, 2H), 2.70-2.55 (m, 4H), |
| | [1-[2-oxo-2-[4-(2,2,2- | 2.04 (s, 3H), 1.96 (s, 3H). |
| | trifluoroethyl)piperazin-1- | ¹⁹F NMR (282 MHz, DMSO-d₆) δ −67.82. |
| | yl]ethyl]pyrazol-3-yl]amino]thiazole-5- | |
| | carboxamide | |
| BAA- 185 | 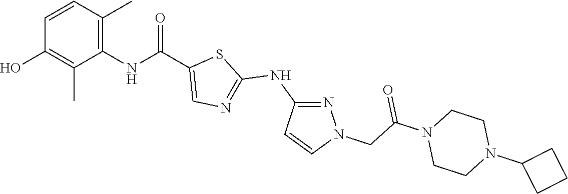 [SMILES: Cc1ccc(O)c(C)c1NC(=O)c1cnc(Nc2ccn(CC(=O)N3CCN(C4CCC4)CC3)n2)s1] | MS (ES+) m/z 510.0 (M + H). ¹H NMR (300 MHz, DMSO-d₆) δ 11.05 (s, 1H), 9.51 (s, 1H), 9.19 (s, 1H), 8.10 (s, 1H), 7.59 (d, J = 2.4 Hz, 1H), 6.88 (d, J = 8.2 Hz, 1H), 6.68 (d, J = 8.2 Hz, 1H), 5.98 (d, J = 2.4 Hz, 1H), 5.06 (s, 2H), 3.70-3.37 (m, 4H), 2.78-2.63 (m, 1H), 2.43-2.10 (m, 4H), 2.07 (s, 3H), 2.04- 1.90 (m, 5H), 1.89-1.54 (m, 4H). ¹H NMR (300 MHz, DMSO-d₆ + D₂O) δ 8.05 (s, 1H), 7.56 (d, J = 2.4 Hz, 1H), 6.88 (d, J = 8.3 Hz, 1H), 6.67 (d, J = 8.2 Hz, 1H), 6.01 (d, J = 2.4 Hz, 1H), 5.01 (s, 2H), 3.62-3.40 (m, 4H), |
| | | 2.80 (s, 1H), 2.46-2.24 (m, 4H), 2.03 (s, 3H), |
| | 2-[1-[2-(4-cyclobutylpiperazin-1-yl)-2- | 1.95 (s, 5H), 1.87-1.71 (m, 2H), 1.66-1.53 |
| | oxo-ethyl]pyrazol-3-yl]amino]-N-(3- | (m, 2H). |
| | hydroxy-2,6-dimethyl-phenyl)thiazole- | |
| | 5-carboxamide | |
| BAA- 186 | 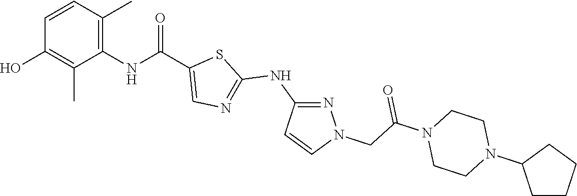 [SMILES: Cc1ccc(O)c(C)c1NC(=O)c1cnc(Nc2ccn(CC(=O)N3CCN(C4CCCC4)CC3)n2)s1] | MS (ES+) m/z 524.0 (M + H). ¹H NMR (300 MHz, DMSO-d₆) δ 11.05 (s, 1H), 9.51 (s, 1H), 9.19 (s, 1H), 8.10 (s, 1H), 7.60 (d, J = 2.4 Hz, 1H), 6.88 (d, J = 8.2 Hz, 1H), 6.68 (d, J = 8.2 Hz, 1H), 5.98 (d, J = 2.3 Hz, 1H), 5.06 (s, 2H), 3.67-3.39 (m, 4H), 2.49-2.30 (m, 5H), 2.07 (s, 3H), 1.99 (s, 3H), 1.86-1.72 (m, 2H), 1.67-1.55 (m, 2H), 1.54-1.44 (m, 2H), 1.41-1.26 (m, 2H). ¹H NMR (300 MHz, DMSO-d₆ + D₂O) δ 8.05 (s, 1H), 7.57 (d, J = 2.4 Hz, 1H), 6.88 (d, J = 8.3 Hz, 1H), 6.66 (d, J = 8.2 Hz, 1H), 6.01 (d, J = |
| | | 2.3 Hz, 1H), 5.00 (s, 2H), 3.61-3.41 (m, 4H), |
| | 2-[ 1-[2-(4-cyclopentylpiperazin-1-yl)- | 2.44 (s, 5H), 2.03 (s, 3H), 1.95 (s, 3H), 1.82- |
| | 2-oxo-ethyl]pyrazol-3-yl]amino]-N-(3- | 1.70 (m, 2H), 1.61-1.51 (m, 2H), 1.48-1.39 |
| | hydroxy-2,6-dimethyl-phenyl)thiazole- | (m, 2H), 1.37-1.26 (m, 2H). |
| | 5-carboxamide | |
| BAA- 187 | 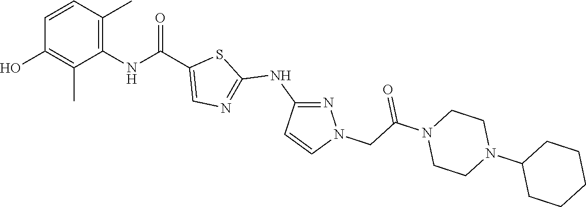 [SMILES: Cc1ccc(O)c(C)c1NC(=O)c1cnc(Nc2ccn(CC(=O)N3CCN(C4CCCCC4)CC3)n2)s1] | MS (ES+) m/z 538.0 (M + H). ¹H NMR (300 MHz, DMSO-d₆) δ 11.05 (s, 1H), 9.51 (s, 1H), 9.19 (s, 1H), 8.10 (s, 1H), 7.60 (d, J = 2.4 Hz, 1H), 6.88 (d, J = 8.2 Hz, 1H), 6.68 (d, J = 8.2 Hz, 1H), 5.98 (d, J = 2.3 Hz, 1H), 5.05 (s, 2H), 3.67-3.39 (m, 4H), 2.58-2.41 (m, 4H), 2.35-2.20 (m, 1H), 2.07 (s, 3H), 1.99 (s, 3H), 1.73 (s, 4H), 1.56 (d, J = 11.7 Hz, 1H), 1.29-1.04 (m, 5H). ¹H NMR (300 MHz, DMSO-d₆ + D₂O) δ 8.06 (s, 1H), 7.57 (d, J = 2.4 Hz, 1H), 6.88 (d, J = 8.3 Hz, 1H), 6.67 (d, J = 8.2 Hz, 1H), 6.01 (d, J = 2.4 Hz, 1H), 4.99 (s, 2H), 3.56-3.39 (m, 4H), |
| | | 2.55 (s, 4H), 2.34-2.22 (m, 1H), 2.03 (s, 3H), |
| | 2-[1-[2-(4-cyclohexylpiperazin-1-yl)- | 1.95 (s, 3H), 1.76-1.63 (m, 4H), 1.51 (d, J = |
| | 2-oxo-ethyl]pyrazol-3-yl]amino]-N-(3- | 11.5 Hz, 1H), 1.21-0.99 (m, 5H). |
| | hydroxy-2,6-dimethyl-phenyl)thiazole- | |
| | 5-carboxamide | |
| BAA- 188 | 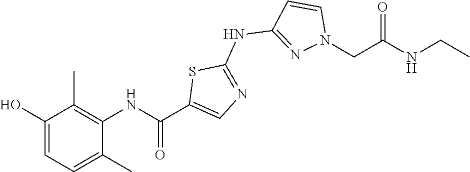 [SMILES: CCNC(=O)Cn1ccc(Nc2ncc(C(=O)Nc3c(C)ccc(O)c3C)s2)n1] | MS (ES+) m/z 414.9 (M + H). ¹H NMR (300 MHz, DMSO-d₆) δ 11.07 (s, 1H), 9.52 (s, 1H), 9.19 (s, 1H), 8.16-8.05 (m, 2H), 7.65 (d, J = 2.3 Hz, 1H), 6.88 (d, J = 8.2 Hz, 1H), 6.68 (d, J = 8.2 Hz, 1H), 5.99 (d, J = 2.3 Hz, 1H), 4.69 (s, 2H), 3.11 (qd, J = 7.2, 5.4 Hz, 2H), 2.07 (s, 3H), 1.99 (s, 3H), 1.04 (t, J = 7.2 Hz, 3H). |
| | 2-[1-[2-(ethylamino)-2-oxo- | |
| | ethyl]pyrazol-3-yl]amino]-N-(3- | |
| | hydroxy-2,6-dimethyl-phenyl)thiazole- | |
| | 5-carboxamide | |
| BAA- 189 | 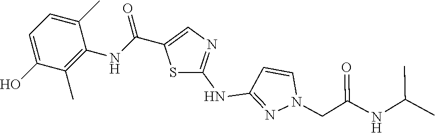 [SMILES: Cc1ccc(O)c(C)c1NC(=O)c1cnc(Nc2ccn(CC(=O)NC(C)C)n2)s1] | MS (ES+) m/z 428.9 (M + H). ¹H NMR (300 MHz, DMSO-d₆) δ 11.07 (s, 1H), 9.52 (s, 1H), 9.19 (s, 1H), 8.10 (s, 1H), 8.06 (d, J = 7.6 Hz, 1H), 7.64 (d, J = 2.3 Hz, 1H), 6.88 (d, J = 8.2 Hz, 1H), 6.68 (d, J = 8.2 Hz, 1H), 5.98 (d, J = 2.3 Hz, 1H), 4.67 (s, 2H), 3.93- 3.75 (m, J = 6.7 Hz, 1H), 2.07 (s, 3H), 1.99 (s, 3H), 1.08 (d, J = 6.6 Hz, 6H). |
| | N-(3-hydroxy-2,6-dimethyl-phenyl)-2- | |
| | [[1-[2-(isopropylamino)-2-oxo- | |
| | ethyl]pyrazol-3-yl]amino]thiazole-5- | |
| | carboxamide | |
| BAA- 190 | 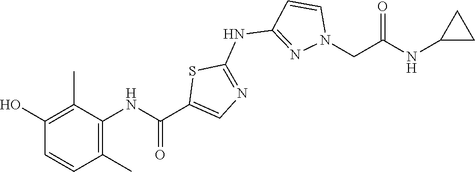 [SMILES: Cc1ccc(O)c(C)c1NC(=O)c1cnc(Nc2ccn(CC(=O)NC3CC3)n2)s1] | MS (ES+) m/z 427.0 (M + H). ¹H NMR (300 MHz, DMSO-d₆) δ 11.06 (s, 1H), 9.52 (s, 1H), 9.19 (s, 1H), 8.28 (d, J = 4.2 Hz, 1H), 8.10 (s, 1H), 7.64 (d, J = 2.4 Hz, 1H), 6.89 (d, J = 8.2 Hz, 1H), 6.68 (d, J = 8.2 Hz, 1H), 5.98 (d, J = 2.3 Hz, 1H), 4.65 (s, 2H), 2.71- 2.60 (m, 1H), 0.68-0.58 (m, 2H), 0.49-0.38 (m, 2H). |
| | 2-[1-[2-(cyclopropylamino)-2-oxo- | |
| | ethyl]pyrazol-3-yl]amino]-N-(3- | |
| | hydroxy-2,6-dimethyl-phenyl)thiazole- | |
| | 5-carboxamide | |
| BAA- 191 | 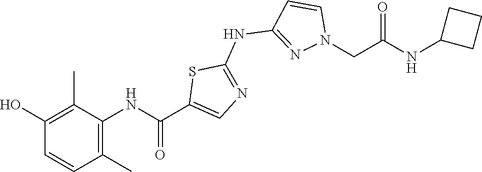 [SMILES: Cc1ccc(O)c(C)c1NC(=O)c1cnc(Nc2ccn(CC(=O)NC3CCC3)n2)s1] | MS (ES+) m/z 441.0 (M + H). ¹H NMR (300 MHz, DMSO-d₆) δ 11.06 (s, 1H), 9.51 (s, 1H), 9.18 (s, 1H), 8.43 (d, J = 7.7 Hz, 1H), 8.10 (s, 1H), 7.63 (d, J = 2.3 Hz, 1H), 6.88 (d, J = 8.2 Hz, 1H), 6.68 (d, J = 8.2 Hz, 1H), 5.98 (d, J = 2.3 Hz, 1H), 4.67 (s, 2H), 4.19 (h, J = 8.1 Hz, 1H), 2.23-2.10 (m, 2H), 2.07 (s, 3H), 1.98 (s, 3H), 1.96-1.84 (m, 2H), 1.70- 1.55 (m, 2H). |
| | 2-[1-[2-(cyclobutylamino)-2-oxo- | |
| | ethyl]pyrazol-3-yl]amino]-N-(3- | |
| | hydroxy-2,6-dimethyl-phenyl)thiazole- | |
| | 5-carboxamide | |
| BAA- 192 | 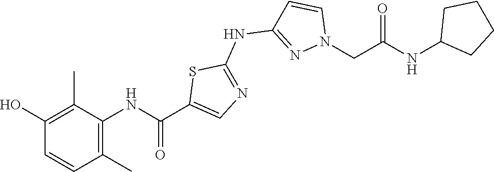 [SMILES: Cc1ccc(O)c(C)c1NC(=O)c1cnc(Nc2ccn(CC(=O)NC3CCCC3)n2)s1] | MS (ES+) m/z 455.1 (M + H). ¹H NMR (300 MHz, DMSO-d₆) δ 11.06 (s, 1H), 9.51 (s, 1H), 9.19 (s, 1H), 8.14 (d, J = 7.2 Hz, 1H), 8.10 (s, 1H), 7.64 (d, J = 2.3 Hz, 1H), 6.88 (d, J = 8.2 Hz, 1H), 6.68 (d, J = 8.2 Hz, 1H), 5.98 (d, J = 2.3 Hz, 1H), 4.68 (s, 2H), 4.00 (h, J = 6.8 Hz, 1H), 2.07 (s, 3H), 1.99 (s, 3H), 1.89- 1.74 (m, 2H), 1.70-1.58 (m, 2H), 1.55-1.35 (m, 4H). |
| | 2-[1-[2-(cyclopentylamino)-2-oxo- | |
| | ethyl]pyrazol-3-yl]amino]-N-(3- | |
| | hydroxy-2,6-dimethyl-phenyl)thiazole- | |
| | 5-carboxamide | |
| BAA- 193 | 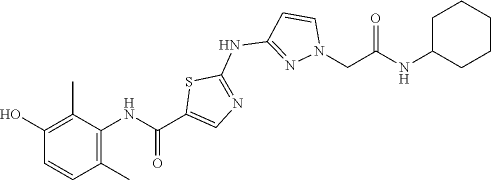 [SMILES: Cc1ccc(O)c(C)c1NC(=O)c1cnc(Nc2ccn(CC(=O)NC3CCCCC3)n2)s1] | MS (ES+) m/z 469.1 (M + H). ¹H NMR (300 MHz, DMSO-d₆) δ 11.06 (s, 1H), 9.51 (s, 1H), 9.19 (s, 1H), 8.10 (s, 1H), 8.04 (d, J = 7.8 Hz, 1H), 7.64 (d, J = 2.3 Hz, 1H), 6.88 (d, J = 8.2 Hz, 1H), 6.68 (d, J = 8.2 Hz, 1H), 5.98 (d, J = 2.3 Hz, 1H), 4.69 (s, 2H), 3.62- 3.46 (m, 1H), 2.07 (s, 3H), 1.99 (s, 3H), 1.81- 1.61 (m, 4H), 1.53 (d, J = 12.0 Hz, 1H), 1.33- 1.09 (m, 5H). |
| | 2-[1-[2-(cyclohexylamino)-2-oxo- | |
| | ethyl]pyrazol-3-yl]amino]-N-(3- | |
| | hydroxy-2,6-dimethyl-phenyl)thiazole- | |
| | 5-carboxamide | |
| BAA- 194 | 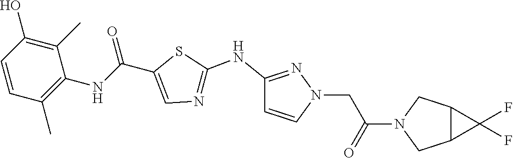 [SMILES: Cc1ccc(O)c(C)c1NC(=O)c1cnc(Nc2ccn(CC(=O)N3CC4C(C3)C4(F)F)n2)s1] | MS (ES+) m/z 489.0 (M + H). ¹H NMR (300 MHz, DMSO-d₆) δ 11.05 (s, 1H), 9.51 (s, 1H), 9.19 (s, 1H), 8.10 (s, 1H), 7.59 (d, J = 2.4 Hz, 1H), 6.88 (d, J = 8.2 Hz, 1H), 6.68 (d, J = 8.2 Hz, 1H), 5.99 (d, J = 2.4 Hz, 1H), 5.05-4.88 (m, 2H), 3.92 (s, 2H), 3.78-3.59 (m, 2H), 2.76-2.55 (m, 2H), 2.07 (s, 3H), 1.98 (s, 3H). ¹⁹F NMR (282 MHz, DMSO) δ −128.68, −129.24, −153.60, −54.16. |
| | 2-[1-[2-(6,6-difluoro-3- | |
| | azabicyclo[3.1.0]hexan-3-yl)-2-oxo- | |
| | ethyl]pyrazol-3-yl]amino]-N-(3- | |
| | hydroxy-2,6-dimethyl-phenyl)thiazole- | |
| | 5-carboxamide | |
| BAA- 195 | 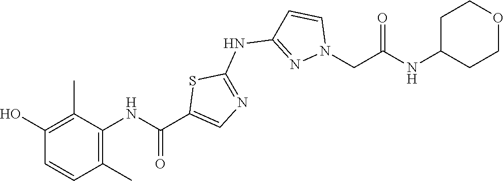 [SMILES: Cc1ccc(O)c(C)c1NC(=O)c1cnc(Nc2ccn(CC(=O)NC3CCOCC3)n2)s1] | MS (ES+) m/z 471.0 (M + H). ¹H NMR (300 MHz, DMSO-d₆) δ 11.06 (s, 1H), 9.51 (s, 1H), 9.19 (s, 1H), 8.21 (d, J = 7.6 Hz, 1H), 8.10 (s, 1H), 7.65 (d, J = 2.3 Hz, 1H), 6.88 (d, J = 8.2 Hz, 1H), 6.68 (d, J = 8.2 Hz, 1H), 5.98 (d, J = 2.3 Hz, 1H), 4.70 (s, 2H), 3.87- 3.69 (m, 3H), 2.07 (s, 3H), 1.98 (s, 3H), 1.81- 1.65 (m, 2H), 1.50-1.31 (m, 2H). ¹H NMR (300 MHz, DMSO-d₆ + D₂O) δ 8.05 (s, 1H), 7.61 (d, J = 2.4 Hz, 1H), 6.88 (d, J = 8.2 Hz, 1H), 6.67 (d, J = 8.2 Hz, 1H), 6.01 (d, J = |
| | | 2.4 Hz, 1H), 4.67 (s, 2H), 3.85-3.70 (m, 3H), |
| | N-(3-hydroxy-2,6-dimethyl-phenyl)-2- | 3.32 (td, J = 11.5, 2.3 Hz, 2H), 2.03 (s, 3H), |
| | [[1-[2-oxo-2-(tetrahydropyran-4- | 1.95 (s, 3H), 1.78-1.63 (m, 2H), 1.50-1.32 |
| | ylamino)ethyl]pyrazol-3- | (m, 2H). |
| | ylamino]thiazole-5-carboxamide | |
| BAA- 196 | 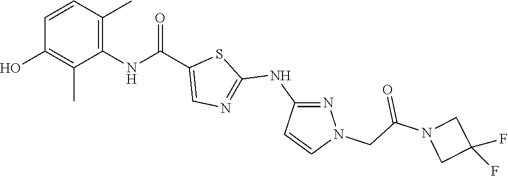 [SMILES: Cc1ccc(O)c(C)c1NC(=O)c1cnc(Nc2ccn(CC(=O)N3CC(F)(F)C3)n2)s1] | MS (ES+) m/z 463.0 (M + H). ¹H NMR (300 MHz, DMSO-d₆) δ 11.10 (s, 1H), 9.52 (s, 1H), 9.19 (s, 1H), 8.10 (s, 1H), 7.63 (d, J = 2.4 Hz, 1H), 6.89 (d, J = 8.2 Hz, 1H), 6.68 (d, J = 8.2 Hz, 1H), 6.02 (d, J = 2.4 Hz, 1H), 4.93 (s, 2H), 4.62 (t, J = 12.5 Hz, 2H), 4.37 (t, J = 12.6 Hz, 2H), 2.07 (s, 3H), 1.99 (s, 3H). ¹⁹F NMR (282 MHz, DMSO) δ −99.55. |
| | 2-[1-[2-(3, 3-difluoroazetidin-1-yl)-2- | |
| | oxo-ethyl]pyrazol-3-yl]amino]-N-(3- | |
| | hydroxy-2,6-dimethyl-phenyl)thiazole- | |
| | 5-carboxamide | |
| BAA- 197 | 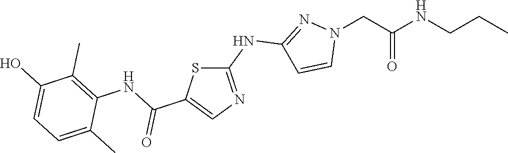 [SMILES: CCCNC(=O)Cn1ccc(Nc2ncc(C(=O)Nc3c(C)ccc(O)c3C)s2)n1] | MS (ES+) m/z 429.3 (M + H). ¹H NMR (300 MHz, DMSO-d₆) δ 11.07 (s, 1H), 9.51 (s, 1H), 9.19 (s, 1H), 8.17-8.02 (m, 2H), 7.65 (d, J = 2.3 Hz, 1H), 6.88 (d, J = 8.2 Hz, 1H), 6.68 (d, J = 8.2 Hz, 1H), 5.99 (d, J = 2.3 Hz, 1H), 4.71 (s, 2H), 3.05 (q, J = 6.8 Hz, 2H), 2.07 (s, 3H), 1.99 (s, 3H), 1.43 (h, J = 7.3 Hz, 2H), 0.85 (t, J = 7.4 Hz, 3H). |
| | N-(3-hydroxy-2,6-dimethyl-phenyl)-2- | |
| | [1-[2-oxo-2- | |
| | (propylamino)ethyl]pyrazol-3- | |
| | yl]amino]thiazole-5-carboxamide | |
| BAA- 198 | 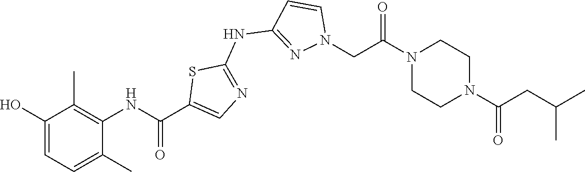 [SMILES: Cc1ccc(O)c(C)c1NC(=O)c1cnc(Nc2ccn(CC(=O)N3CCN(C(=O)CC(C)C)CC3)n2)s1] | MS (ES+) m/z 540.4 (M + H). ¹H NMR (300 MHz, DMSO-d₆) δ 11.06 (s, 1H), 9.51 (s, 1H), 9.19 (s, 1H), 8.10 (s, 1H), 7.60 (d, J = 2.3 Hz, 1H), 6.88 (d, J = 8.2 Hz, 1H), 6.68 (d, J = 8.2 Hz, 1H), 5.99 (d, J = 2.3 Hz, 1H), 5.10 (s, 2H), 3.62-3.42 (m, 8H), 2.27-2.17 (m, 2H), 2.07 (s, 3H), 2.03-1.93 (m, 4H), 0.89 (d, J = 6.6 Hz, 6H). |
| | N-(3-hydroxy-2,6-dimethyl-phenyl)-2- | |
| | [[1-[2-[4-(3-methylbutanoyl) piperazin- | |
| | 1-yl]-2-oxo-ethyl]pyrazol-3- | |
| | yl]amino]thiazole-5-carboxamide | |
| BAA- 199 | 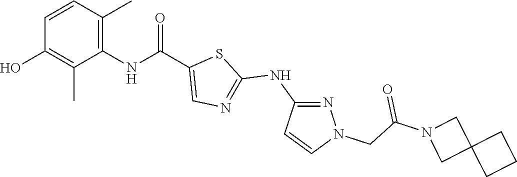 [SMILES: Cc1ccc(O)c(C)c1NC(=O)c1cnc(Nc2ccn(CC(=O)N3CC4(CCC4)C3)n2)s1] | MS (ES+) m/z 467.3 (M + H). ¹H NMR (300 MHz, DMSO-d₆) δ 11.07 (s, 1H), 9.51 (s, 1H), 9.19 (s, 1H), 8.10 (s, 1H), 7.62 (d, J = 2.4 Hz, 1H), 6.89 (d, J = 8.2 Hz, 1H), 6.68 (d, J = 8.2 Hz, 1H), 5.98 (d, J = 2.3 Hz, 1H), 4.74 (s, 2H), 4.14 (s, 2H), 3.87 (s, 2H), 2.18- 2.08 (m, 4H), 2.07 (s, 3H), 1.99 (s, 3H), 1.82- 1.69 (m, 2H). |
| | 2-[1-[2-(2-azaspiro[3.3]heptan-2-yl)- | |
| | 2-oxo-ethyl]pyrazol-3-yl]amino]-N-(3- | |
| | hydroxy-2,6-dimethyl-phenyl)thiazole- | |
| | 5-carboxamide | |

### 2-[[1-[2-(2,6-Diazaspiro[3.3]heptan-2-yl)-2-oxo-ethyl]pyrazol-3-yl]amino]-N-(3-hydroxy-2,6-dimethyl-phenyl)thiazole-5-carboxamide 2,2,2-trifluoroacetate (BAA-200)

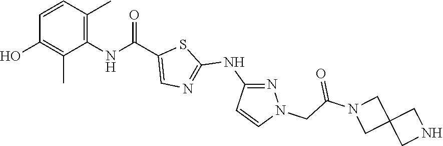 [SMILES: Cc1ccc(O)c(C)c1NC(=O)c1cnc(Nc2ccn(CC(=O)N3CC4(CNC4)C3)n2)s1]

To a solution of tert-butyl 6-[2-[3-[5-[(3-hydroxy-2,6-dimethyl-phenyl) carbamoyl]thiazol-2-yl]amino]pyrazol-1-yl]acetyl]-2,6-diazaspiro[3.3]heptane-2-carboxylate (10 mg, 0.0176 mmol, 1.0 eq) in DCM (2 mL) was added TFA (0.050 mL, 0.649 mmol, 37 eq) at 0° C. and stirred at RT for 4 h. The mixture was concentrated in vacuo, the residue was triturated with Et₂O, filtered, washed with Et₂O and dried in vacuo at 50° C. to afford the title compound (BAA-200)(9.0 mg, 0.0155 mmol, 88%) as a white solid. MS (ES+) m/z 467.9 (M+H). ¹H NMR (300 MHz, DMSO-d₆) δ 11.23-10 11.03 (br s, 1H), 9.55 (s, 1H), 9.32-9.12 (br s, 1H), 8.59-8.39 (br s, 2H), 8.13 (s, 1H), 7.63 (d, J=2.4 Hz, 1H), 6.89 (d, J=8.2 Hz, 1H), 6.69 (d, J=8.1 Hz, 1H), 5.97 (d, J=2.3 Hz, 1H), 4.75 (s, 2H), 4.34 (s, 2H), 4.23-3.98 (m, 6H), 2.07 (s, 3H), 1.99 (s, 3H). ¹H NMR (300 MHz, DMSO-d₆+D2O) δ 8.07 (s, 1H), 7.58 (s, 1H), 6.89 (d, J=8.2 Hz, 1H), 6.67 (d, J=8.2 Hz, 1H), 6.01 (d, J=2.3 Hz, 1H), 4.70 (s, 2H), 4.35 (s, 2H), 4.12 (s, 6H), 2.03 (s, 3H), 1.95 (s, 3H).

Amide Formation from BAA-124 and Other Phenol 5-Substituted Pyrazolyl Acetic Acids with Amines

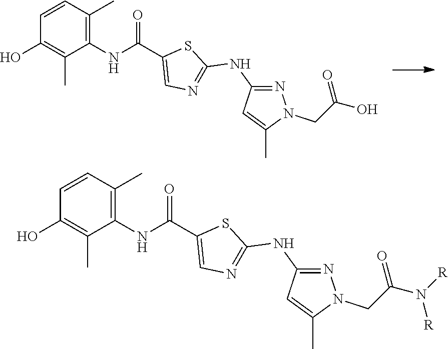

The following example compounds were prepared as described in Method L from BAA-124:

| # | Structure & Name | Analytical Data |
|---|---|---|
| BAA- 201 | 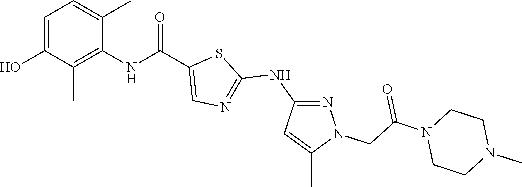 [SMILES: Cc1ccc(O)c(C)c1NC(=O)c1cnc(Nc2cc(C)n(CC(=O)N3CCN(C)CC3)n2)s1] | MS (ES+) m/z 484.1 (M + H). ¹H NMR (300 MHz, DMSO-d₆) δ 10.95 (s, 1H), 9.49 (s, 1H), 9.19 (s, 1H), 8.09 (s, 1H), 6.88 (d, J = 8.2 Hz, 1H), 6.68 (d, J = 8.2 Hz, 1H), 5.81 (s, 1H), 5.00 (s, 2H), 3.56-3.43 (m, 4H), 2.38-2.24 (m, 4H), 2.19 (s, 3H), 2.14 (s, 3H), 2.07 (s, 3H), 1.98 (s, 3H). |
| | N-(3-hydroxy-2,6-dimethyl-phenyl)-2- | |
| | [5-methyl-1-[2-(4-methylpiperazin-1- | |
| | yl)-2-oxo-ethyl]pyrazol-3- | |
| | yl]amino]thiazole-5-carboxamide | |
| BAA- 202 | 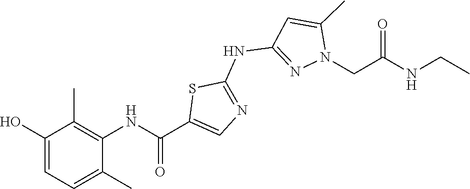 [SMILES: CCNC(=O)Cn1nc(Nc2ncc(C(=O)Nc3c(C)ccc(O)c3C)s2)cc1C] | MS (ES+) m/z 429.0 (M + H). ¹H NMR (300 MHz, DMSO-d₆) δ 10.97 (s, 1H), 9.50 (s, 1H), 9.19 (s, 1H), 8.16-8.04 (m, 2H), 6.88 (d, J = 8.2 Hz, 1H), 6.68 (d, J = 8.2 Hz, 1H), 5.82 (d, J = 0.8 Hz, 1H), 4.63 (s, 2H), 3.16-3.06 (m, 2H), 2.21 (s, 3H), 2.07 (s, 3H), 1.98 (s, 3H), 1.04 (t, J = 7.2 Hz, 3H). |
| | 2-[[1-[2-(ethylamino)-2-oxo-ethyl]-5- | |
| | methyl-pyrazol-3-yl]amino]-N-(3- | |
| | hydroxy-2,6-dimethyl-phenyl)thiazole- | |
| | 5-carboxamide | |
| BAA- 203 | 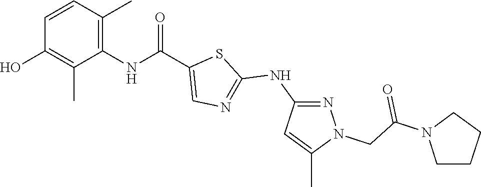 [SMILES: Cc1ccc(O)c(C)c1NC(=O)c1cnc(Nc2cc(C)n(CC(=O)N3CCCC3)n2)s1] | MS (ES+) m/z 455.0 (M + H). ¹H NMR (300 MHz, DMSO-d₆) δ 10.96 (s, 1H), 9.50 (s, 1H), 9.19 (s, 1H), 8.09 (s, 1H), 6.88 (d, J = 8.2 Hz, 1H), 6.68 (d, J = 8.2 Hz, 1H), 5.80 (s, 1H), 4.88 (s, 2H), 3.55 (t, J = 6.7 Hz, 2H), 2.16 (s, 3H), 2.06 (s, 3H), 1.98 (s, 3H), 1.96-1.87 (m, 2H), 1.79 (p, J = 6.3 Hz, 2H). |
| | N-(3-hydroxy-2,6-dimethyl-phenyl)-2- | |
| | [5-methyl-1-(2-oxo-2-pyrrolidin-1-yl- | |
| | ethyl)pyrazol-3-yl]amino]thiazole-5- | |
| | carboxamide | |
| BAA- 204 | 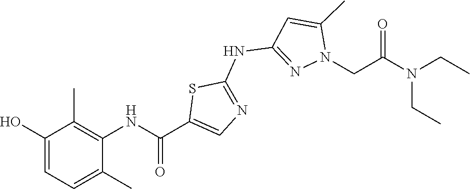 [SMILES: CCN(CC)C(=O)Cn1nc(Nc2ncc(C(=O)Nc3c(C)ccc(O)c3C)s2)cc1C] | MS (ES+) m/z 457.1 (M + H). ¹H NMR (300 MHz, DMSO-d₆) δ 10.94 (s, 1H), 9.49 (s, 1H), 9.19 (s, 1H), 8.09 (s, 1H), 6.88 (d, J = 8.2 Hz, 1H), 6.68 (d, J = 8.2 Hz, 1H), 5.82 (s, 1H), 4.94 (s, 2H), 3.47-3.38 (m, 2H), 3.32-3.25 (m, 2H), 2.15 (s, 3H), 2.06 (s, 3H), 1.98 (s, 3H), 1.17 (t, J = 7.0 Hz, 3H), 1.03 (t, J = 7.0 Hz, 3H). |
| | 2-[[1-[2-(diethylamino)-2-oxo-ethyl]-5- | |
| | methyl-pyrazol-3-yl]amino]-N-(3- | |
| | hydroxy-2,6-dimethyl-phenyl)thiazole- | |
| | 5-carboxamide | |

Amide Formation from BAA-125 and Other Phenol 4-Substituted Pyrazolyl Acetic Acids with Amines

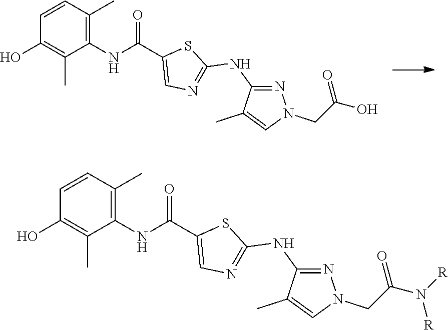

The following example compounds were prepared as described in Method L from BAA-125:

| # | Structure & Name | Analytical Data |
|---|---|---|
| BAA-205 | 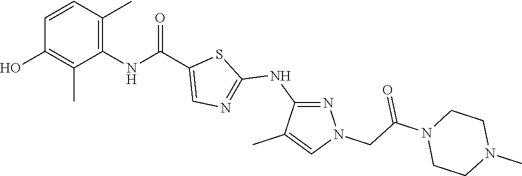 [SMILES: Cc1cn(CC(=O)N2CCN(C)CC2)nc1Nc1ncc(C(=O)Nc2c(C)ccc(O)c2C)s1] | MS (ES+) m/z 484.0 (M + H). ¹H NMR (300 MHz, DMSO-d₆) δ 10.71 (s, 1H), 9.48 (s, 1H), 9.18 (s, 1H), 8.10 (s, 1H), 7.40 (d, J = 1.0 Hz, 1H), 6.88 (d, J = 8.2 Hz, 1H), 6.68 (d, J = 8.1 Hz, 1H), 4.99 (s, 2H), 3.48 (dt, J = 19.7, 5.0 Hz, 4H), 2.31 (dt, J = 20.5, 5.1 Hz, 4H), 2.18 (s, 3H), 2.07 (s, 3H), 2.01 (s, 3H), 1.99 (s, 3H). |
| | N-(3-hydroxy-2,6-dimethyl-phenyl)-2- | |
| | [[4-methyl-1-[2-(4-methylpiperazin-1- | |
| | yl)-2-oxo-ethyl]pyrazol-3- | |
| | yl]amino]thiazole-5-carboxamide | |
| BAA-206 | 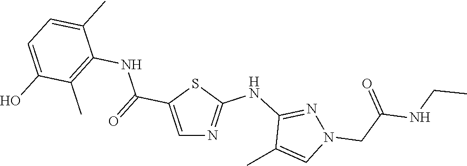 [SMILES: CCNC(=O)Cn1cc(C)c(Nc2ncc(C(=O)Nc3c(C)ccc(O)c3C)s2)n1] | MS (ES+) m/z 429.1 (M + H). ¹H NMR (300 MHz, DMSO-d₆) δ 10.73 (s, 1H), 9.49 (s, 1H), 9.19 (s, 1H), 8.11 (s, 1H), 8.06 (t, J = 5.5 Hz, 1H), 7.46 (d, J = 1.0 Hz, 1H), 6.88 (d, J = 8.2 Hz, 1H), 6.68 (d, J = 8.2 Hz, 1H), 4.63 (s, 2H), 3.18-3.03 (m, 2H), 2.07 (s, 3H), 2.01 (s, 3H), 1.98 (s, 3H), 1.03 (t, J = 7.2 Hz, 3H). |
| | 2-[[1-[2-(ethylamino)-2-oxo-ethyl]-4- | |
| | methyl-pyrazol-3-yl]amino]-N-(3- | |
| | hydroxy-2,6-dimethyl-phenyl)thiazole- | |
| | 5-carboxamide | |
| BAA-207 | 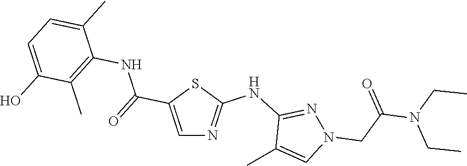 [SMILES: CCN(CC)C(=O)Cn1cc(C)c(Nc2ncc(C(=O)Nc3c(C)ccc(O)c3C)s2)n1] | MS (ES+) m/z 457.1 (M + H). ¹H NMR (300 MHz, DMSO-d₆) δ 10.69 (s, 1H), 9.47 (s, 1H), 9.18 (s, 1H), 8.10 (s, 1H), 7.43 (d, J = 0.9 Hz, 1H), 6.88 (d, J = 8.2 Hz, 1H), 6.68 (d, J = 8.2 Hz, 1H), 4.94 (s, 2H), 3.41 (q, J = 7.2 Hz, 2H), 3.28 (q, J = 7.1 Hz, 2H), 2.07 (s, 3H), 2.01 (s, 3H), 1.99 (s, 3H), 1.16 (t, J = 7.1 Hz, 3H), 1.03 (t, J = 7.0 Hz, 3H). |
| | 2-[[1-[2-(diethylamino)-2-oxo-ethyl]-4- | |
| | methyl-pyrazol-3-yl]amino]-N-(3- | |
| | hydroxy-2,6-dimethyl-phenyl)thiazole- | |
| | 5-carboxamide | |
| BAA-208 | 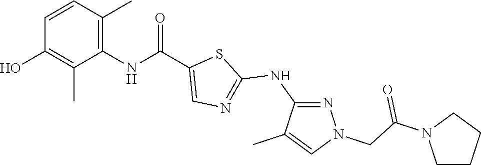 [SMILES: Cc1cn(CC(=O)N2CCCC2)nc1Nc1ncc(C(=O)Nc2c(C)ccc(O)c2C)s1] | MS (ES+) m/z 455.0 (M + H). ¹H NMR (300 MHz, DMSO-d₆) δ 10.72 (s, 1H), 9.48 (s, 1H), 9.19 (s, 1H), 8.11 (s, 1H), 7.41 (d, J = 0.9 Hz, 1H), 6.88 (d, J = 8.2 Hz, 1H), 6.68 (d, J = 8.2 Hz, 1H), 4.88 (s, 2H), 3.53 (t, J = 6.7 Hz, 2H), 3.34-3.27 (m, 2H), 2.07 (s, 3H), 2.02 (s, 3H), 1.99 (s, 3H), 1.96-1.87 (m, 2H), 1.78 (p, J = 6.4 Hz, 2H). |
| | N-(3-hydroxy-2,6-dimethyl-phenyl)-2- | |
| | [[4-methyl-1-(2-oxo-2-pyrrolidin-1-yl- | |
| | ethyl)pyrazol-3-yl]amino]thiazole-5- | |
| | carboxamide | |
| BAA-374 | 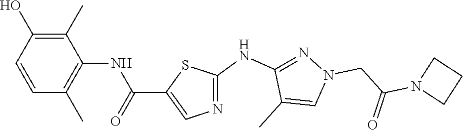 [SMILES: Cc1cn(CC(=O)N2CCC2)nc1Nc1ncc(C(=O)Nc2c(C)ccc(O)c2C)s1] | MS (ES+) m/z 441 (M + H). ¹H NMR (300 MHz, MeOD) δ 8.03 (s, 1H), 7.39 (d, J = 0.9 Hz, 1H), 6.91 (d, J = 8.2 Hz, 1H), 6.68 (d, J = 8.2 Hz, 1H), 4.70 (s, 2H), 4.36-4.24 (m, 2H), 4.07 (t, J = 7.8 Hz, 2H), 2.35 (p, J = 7.9 Hz, 2H), 2.16 (d, J = 1.4 Hz, 3H), 2.11 (s, 3H), 2.07 (d, J = 0.8 Hz, 3H). |
| | 2-((1-(2-(azetidin-1-yl)-2-oxoethyl)-4- | |
| | methyl-1H-pyrazol-3-yl)amino)-N-(3- | |
| | hydroxy-2,6-dimethylphenyl)thiazole-5- | |
| | carboxamide | |
| BAA-381 | 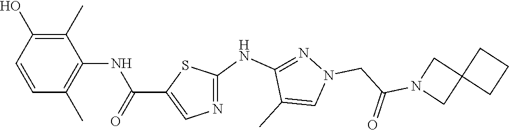 [SMILES: Cc1cn(CC(=O)N2CC3(CCC3)C2)nc1Nc1ncc(C(=O)Nc2c(C)ccc(O)c2C)s1] | MS (ES+) m/z 481 (M + H). ¹H NMR (300 MHz, MeOD) δ 8.02 (s, 1H), 7.38 (d, J = 0.9 Hz, 1H), 6.91 (d, J = 8.3 Hz, 1H), 6.88 (d, J = 8.2 Hz, 1H), 4.69 (s, 2H), 4.21 (s, 2H), 3.99 (s, 2H), 2.25-2.16 (m, 4H), 2.16 (d, J = 0.7 Hz, 3H), 2.11 (s, 3H), 2.07 (d, J = 0.8 Hz, 3H), 1.82 (p, J = 7.4 Hz, 2H). |
| | N-(3-hydroxy-2,6-dimethylphenyl)-2- | |
| | ((4-methyl-1-(2-oxo-2-(2- | |
| | azaspiro[3.3]heptan-2-yl)ethyl)-1H- | |
| | pyrazol-3-yl)amino)thiazole-5- | |
| | carboxamide | |
| BAA-595 | 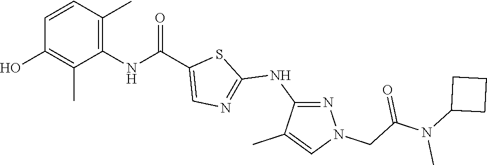 [SMILES: Cc1cn(CC(=O)N(C)C2CCC2)nc1Nc1ncc(C(=O)Nc2c(C)ccc(O)c2C)s1] | ¹H NMR (400 MHz, DMSO-d₆) δ 8.35 (s, 1H), 8.02 (s, 1H), 7.35 (s, 1H), 6.91 (d, J = 8.2 Hz, 1H), 6.68 (d, J = 8.2 Hz, 1H), 4.98 (d, J = 15.0 Hz, 2H), 4.65-4.54 (m, 0.5H), 3.09 (s, 1.4H), 2.95 (s, 1.6H), 2.38- 2.18 (m, 2H), 2.15 (s, 3H), 2.10 (s, 3H), 2.07 (s, 3H), 1.78-1.65 (m, 2H). MS (ES⁺) m/z 469.4 (M + H)⁺. |
| | 2-((1-(2-(cyclobutyl(methyl)amino)-2- | |
| | oxoethyl)-4-methyl-1H-pyrazol-3- | |
| | yl)amino)-N-(3-hydroxy-2,6- | |
| | dimethylphenyl)thiazole-5-carboxamide | |

### 2-((6-(2-(Cyclopropyl(ethyl)amino)-2-oxoethyl)pyridin-2-yl)amino)-N-(3-hydroxy-2,6-dimethylphenyl)thiazole-5-carboxamide( ) BAA-426

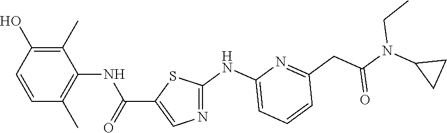 [SMILES: CCN(C(=O)Cc1cccc(Nc2ncc(C(=O)Nc3c(C)ccc(O)c3C)s2)n1)C1CC1]

To a mixture of 2-(6-((5-((3-hydroxy-2,6-dimethylphenyl)carbamoyl)thiazol-2-yl)amino)pyridin-2-yl) acetic acid (60 mg, 0.151 mmol, 1.0 eq), N-ethylcyclopropanamine hydrochloride (22 mg, 0.181 mmol, 1.2 eq) and 2-(7-aza-1H-benzotriazole-1-yl)-1,1,3,3-tetramethyluronium hexafluorophosphate (HATU)(69 mg, 0.181 mmol, 1.2 eq) in DMF (2 mL) at rt was added N, N-diisopropylethylamine (0.13 mL, 0.753 mmol, 5 eq) and the reaction stirred for 16 h at rt. Ethyl acetate (5 mL) and water (5 mL) were added. The phases were separated and the aqueous phase extracted with ethyl acetate (2×5 mL). Combined organic phases were washed with brine (20 mL), dried over MgSO₄, filtered and concentrated in vacuo. Column chromatography (dichloromethane:methanol) gave 2-[6-[2-[cyclopropyl(ethyl)amino]-2-oxo-ethyl]-2-pyridyl]amino]-N-(3-hydroxy-2,6-dimethyl-phenyl)thiazole-5-carboxamide (5.5 mg, 0.0118 mmol, 7.8%). MS (ES+) m/z 466 (M+H). ¹H NMR (300 MHz, MeOD) δ 8.13 (s, 1H), 7.74-7.63 (m, 1H), 7.00-6.88 (m, 3H), 6.69 (d, J=8.2 Hz, 1H), 4.19 (s, 2H), 3.55 (q, J=7.2 Hz, 2H), 2.96 (t, J=4.7 Hz, 1H), 2.17 (s, 3H), 2.12 (s, 3H), 1.14 (t, J=7.1 Hz, 3H), 0.98 (d, J=7.8 Hz, 4H).

### 2-((6-(2-(Dicyclopropylamino)-2-oxoethyl)pyridin-2-yl)amino)-N-(3-hydroxy-2,6-dimethylphenyl)thiazole-5-carboxamide( ) BAA-427

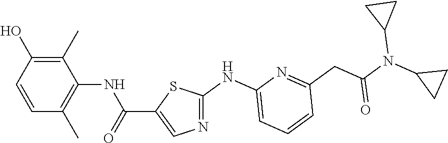 [SMILES: Cc1ccc(O)c(C)c1NC(=O)c1cnc(Nc2cccc(CC(=O)N(C3CC3)C3CC3)n2)s1]

To a mixture of 2-(6-((5-((3-hydroxy-2,6-dimethylphenyl)carbamoyl)thiazol-2-yl)amino)pyridin-2-yl) acetic acid (60 mg, 0.151 mmol, 1 eq) and 2-(7-aza-1H-benzotriazole-1-yl)-1, 1,3,3-tetramethyluronium hexafluorophosphate (HATU)(69 mg, 0.181 mmol, 1.20 eq) in DMF (2 mL) at rt was added 1,8-diazabicyclo[5.4.0]undec-7-ene (0.068 mL, 0.452 mmol, 3 eq) and the reaction stirred for 16 h at rt. Ethyl acetate (5 mL) and water (5 mL) were added. The phases were separated and the aqueous phase extracted with ethyl acetate (2×5 mL). Combined organic phases were washed with brine (20 mL), dried over MgSO₄, filtered and concentrated in vacuo. Column chromatography (dichloromethane:methanol) followed by reverse phase column chromatography (water:acetonitrile) gave 2-[[6-[2-(dicyclopropylamino)-2-oxo-ethyl]-2-pyridyl]amino]-N-(3-hydroxy-2,6-dimethyl-phenyl)thiazole-5-carboxamide (2.7 mg, 0.00565 mmol, 3.8%). MS (ES+) m/z 478 (M+H).¹H NMR (300 MHz, MeOD) δ 8.14 (s, 1H), 7.74-7.63 (m, 1H), 6.94 (t, J=7.7 Hz, 3H), 6.70 (d, J=8.2 Hz, 1H), 4.14 (s, 2H), 2.98-2.59 (m, 2H), 2.19-2.16 (m, 3H), 2.13 (s, 3H), 1.08-0.87 (m, 4H), 0.85-0.64 (m, 4H).

### 2-((6-(2-(Cyclopropyl(methyl)amino)-2-oxoethyl)pyridin-2-yl)amino)-N-(3-hydroxy-2,6-dimethylphenyl)thiazole-5-carboxamide( ) BAA-428

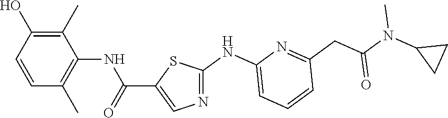 [SMILES: Cc1ccc(O)c(C)c1NC(=O)c1cnc(Nc2cccc(CC(=O)N(C)C3CC3)n2)s1]

To a mixture of 2-[6-[5-[(3-hydroxy-2,6-dimethyl-phenyl) carbamoyl]thiazol-2-yl]amino]-2-pyridyl]acetic acid (42 mg, 0.105 mmol, 1 eq) and 2-(7-aza-1H-benzotriazole-1-yl)-1,1,3,3-tetramethyluronium hexafluorophosphate (HATU)(48 mg, 0.126 mmol, 1.2 eq) in DMF (2 mL) at rt was added 1,8-diazabicyclo[5.4.0]undec-7-ene (0.047 mL, 0.316 mmol, 3.00 eq) and the reaction stirred for 16 h at rt. Ethyl acetate (5 mL) and water (5 mL) were added. The phases were separated and the aqueous phase extracted with ethyl acetate (2×5 mL). Combined organic phases were washed with brine (20 mL), dried over MgSO₄, filtered and concentrated in vacuo. Column chromatography (dichloromethane:methanol) followed by reverse phase column chromatography (water:acetonitrile) gave 2-[6-[2-[cyclopropyl(methyl)amino]-2-oxo-ethyl]-2-pyridyl]amino]-N-(3-hydroxy-2,6-dimethyl-phenyl)thiazole-5-carboxamide (2.8 mg, 0.00620 mmol, 5.9%). MS (ES+) m/z 452 (M+H). ¹H NMR (300 MHz, MeOD) δ 8.13 (s, 1H), 7.74-7.63 (m, 1H), 7.00-6.88 (m, 3H), 6.69 (d, J=8.2 Hz, 1H), 4.21 (s, 2H), 3.08 (dd, J=3.3, 1.7 Hz, 1H), 3.06 (s, 3H), 2.18 (s, 3H), 2.13 (s, 3H), 1.08-0.97 (m, 2H), 0.98-0.86 (m, 2H).

### Boc Deprotections

The following compounds were prepared similarly using Method E:

| # | Name & Structure | Analytical Data |
|---|---|---|
| BAA- 222 | 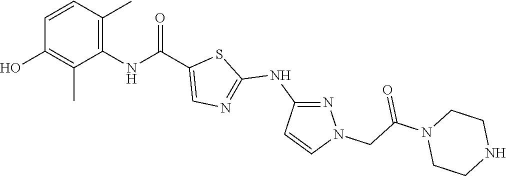 [SMILES: Cc1ccc(O)c(C)c1NC(=O)c1cnc(Nc2ccn(CC(=O)N3CCNCC3)n2)s1] N-(3-hydroxy-2,6-dimethylphenyl)-2-((1- (2-oxo-2-(piperazin-1-yl)ethyl)-1H- pyrazol-3-yl)amino)thiazole-5- carboxamide | MS (ES+) m/z 455.9 (M + H). ¹H NMR (300 MHz, DMSO-d₆) δ 11.47- 11.07 (br s, 1H), 9.61 (s, 1H), 9.36-9.16 (br s, 3H), 8.16 (s, 1H), 7.58 (d, J = 2.4 Hz, 1H), 6.88 (d, J = 8.2 Hz, 1H), 6.70 (d, J = 8.2 Hz, 1H), 6.01 (d, J = 2.4 Hz, 1H), 5.16 (s, 2H), 3.80-3.64 (m, 4H), 3.22-3.02 (m, 4H), 2.07 (s, 3H), 1.98 (s, 3H). ¹H NMR (300 MHz, DMSO-d₆ + D₂O) δ 8.08 (s, 1H), 7.57 (d, J = 2.4 Hz, 1H), 6.88 (d, J = 8.3 Hz, 1H), 6.67 (d, J = 8.2 Hz, 1H), 6.03 (d, J = 2.4 Hz, 1H), 5.10 (s, 2H), 3.77-3.61 (m, 4H), 3.19-3.04 (m, 4H), 2.03 (s, 3H), 1.95 (s, 3H). |
| BAA- 223 | 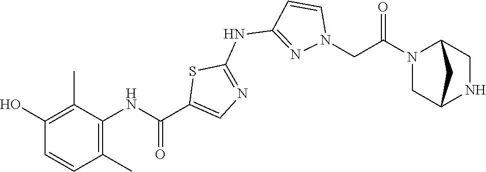 [SMILES: Cc1ccc(O)c(C)c1NC(=O)c1cnc(Nc2ccn(CC(=O)N3C[C@@H]4C[C@H]3CN4)n2)s1] 2-((1-(2-((1R,4R)-2,5- diazabicyclo[2.2.1]heptan-2-yl)-2- oxoethyl)-1H-pyrazol-3-yl)amino)-N-(3- hydroxy-2,6-dimethylphenyl)thiazole-5- carboxamide | MS (ES+) m/z 467.9 (M + H). ¹H NMR (300 MHz, DMSO-d₆) δ 11.43- 11.03 (br s, 1H), 9.61 (s, 1H), 9.67-9.47 (br s, 1H), 9.16-8.96 (br s, 2H), 8.16 (s, 1H), 7.64 (dd, J = 4.1, 2.4 Hz, 1H), 6.88 (d, J = 8.2 Hz, 1H), 6.70 (d, J = 8.2 Hz, 1H), 6.01 (d, J = 2.3 Hz, 1H), 5.21-4.73 (m, 3H), 4.44 (d, J = 26.8 Hz, 2H), 3.89-3.17 (m, 4H), 2.07 (s, 3H), 1.99 (s, 3H), 1.98- 1.79 (m, 2H). ¹H NMR (300 MHz, DMSO-d₆ + D₂O) δ 8.07 (s, 1H), 7.59 (dd, J = 8.2, 2.4 Hz, 1H), 6.89 (d, J = 8.3 Hz, 1H), 6.67 (d, J = 8.2 Hz, 1H), 6.04 (d, J = 2.4 Hz, 1H), 5.18- 4.66 (m, 3H), 4.41 (d, J = 21.0 Hz, 1H), 3.70 (s, 1H), 3.44 (s, 1H), 3.38-3.19 (m, 2H), 2.17-1.78 (m, 8H). |
| BAA- 224 | 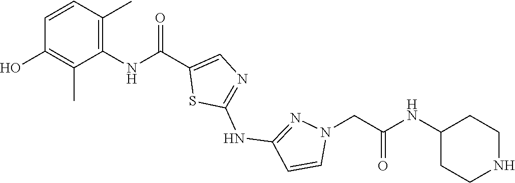 [SMILES: Cc1ccc(O)c(C)c1NC(=O)c1cnc(Nc2ccn(CC(=O)NC3CCNCC3)n2)s1] N-(3-hydroxy-2,6-dimethylphenyl)-2-((1- (2-oxo-2-(piperidin-4-ylamino)ethyl)-1H- pyrazol-3-yl)amino)thiazole-5- carboxamide | MS (ES+) m/z 469.9 (M + H). ¹H NMR (300 MHz, DMSO-d₆) δ 11.41- 11.01 (br s, 1H), 9.60 (s, 1H), 9.39-8.99 (br s, 1H), 8.88-8.68 (br s, 2H), 8.48 (d, J = 7.5 Hz, 1H), 8.16 (s, 1H), 7.66 (d, J = 2.4 Hz, 1H), 6.88 (d, J = 8.2 Hz, 1H), 6.70 (d, J = 8.2 Hz, 1H), 5.99 (d, J = 2.3 Hz, 1H), 4.73 (s, 2H), 3.94-3.79 (m, 1H), 3.31-3.18 (m, 2H), 3.05-2.92 (m, 2H), 2.07 (s, 3H), 1.99 (s, 3H), 1.96-1.88 (m, 2H), 1.74-1.55 (m, 2H). ¹H NMR (300 MHz, DMSO-d₆ + D₂O) δ 8.45 (d, J = 7.2 Hz, 1H), 8.06 (s, 1H), 7.61 (s, 1H), 6.88 (d, J = 8.3 Hz, 1H), 6.66 (d, J = 8.3 Hz, 1H), 6.01 (d, J = 2.4 Hz, 1H), 4.69 (s, 2H), 3.87-3.73 (m, 1H), 3.29- 3.16 (m, 2H), 3.01-2.87 (m, 2H), 2.03 (s, 3H), 1.94 (s, 5H), 1.70-1.50 (m, 2H). |
| BAA- 225 | 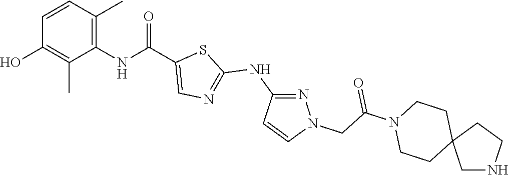 [SMILES: Cc1ccc(O)c(C)c1NC(=O)c1cnc(Nc2ccn(CC(=O)N3CCC4(CCNC4)CC3)n2)s1] N-(3-hydroxy-2,6-dimethylphenyl)-2-((1- (2-oxo-2-(2,8-diazaspiro[4.5]decan-8- yl)ethyl)-1H-pyrazol-3-yl)amino)thiazole- 5-carboxamide | MS (ES+) m/z 510.0 (M + H). ¹H NMR (300 MHz, DMSO-d₆) δ 11.25- 11.05 (br s, 1H), 9.57 (s, 1H), 9.27-9.07 (br s, 3H), 8.14 (s, 1H), 7.60 (d, J = 2.3 Hz, 1H), 6.88 (d, J = 8.2 Hz, 1H), 6.69 (d, J = 8.2 Hz, 1H), 5.98 (d, J = 2.4 Hz, 1H), 5.07 (s, 2H), 3.55-3.40 (m, 4H), 3.28-3.19 (m, 2H), 3.01 (s, 2H), 2.07 (s, 3H), 1.98 (s, 3H), 1.82 (t, J = 7.4 Hz, 2H), 1.66-1.56 (m, 2H), 1.57-1.46 (m, 2H). ¹H NMR (300 MHz, DMSO-d₆ + D₂O) δ 8.06 (s, 1H), 7.56 (d, J = 2.4 Hz, 1H), 6.88 (d, J = 8.3 Hz, 1H), 6.67 (d, J = 8.2 Hz, 1H), 6.01 (d, J = 2.4 Hz, 1H), 5.01 (s, 2H), 3.58-3.34 (m, 4H), 3.22 (t, J = 7.4 Hz, 2H), 3.00 (s, 2H), 2.03 (s, 3H), 1.95 (s, 3H), 1.83 (t, J = 7.4 Hz, 2H), 1.65-1.53 (m, 2H), 1.54-1.43 (m, 2H). |
| BAA- 226 | 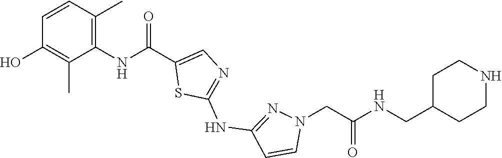 [SMILES: Cc1ccc(O)c(C)c1NC(=O)c1cnc(Nc2ccn(CC(=O)NCC3CCNCC3)n2)s1] N-(3-hydroxy-2,6-dimethylphenyl)-2-((1- (2-oxo-2-((piperidin-4- ylmethyl)amino)ethyl-1H-pyrazol-3- yl)amino)thiazole-5-carboxamide | MS (ES+) m/z (M + H). ¹H NMR (300 MHz, DMSO-d₆) δ 11.50- 11.10 (br s, 1H), 9.62 (s, 1H), 9.43-9.03 (br s, 1H), 8.83 (d, J = 11.3 Hz, 1H), 8.52 (d, J = 11.0 Hz, 1H), 8.29 (t, J = 5.9 Hz, 1H), 8.18 (s, 1H), 7.68 (d, J = 2.3 Hz, 1H), 6.88 (d, J = 8.2 Hz, 1H), 6.70 (d, J = 8.2 Hz, 1H), 5.98 (d, J = 2.3 Hz, 1H), 4.73 (s, 2H), 3.21 (d, J = 12.1 Hz, 2H), 3.01 (t, J = 6.4 Hz, 2H), 2.78 (q, J = 11.6 Hz, 2H), 2.07 (s, 3H), 1.99 (s, 3H), 1.86-1.75 (m, 2H), 1.75-1.64 (m, 1H), 1.38-1.18 (m, 2H). ¹H NMR (300 MHz, DMSO-d₆ + D₂O) δ 8.26 (t, J = 6.1 Hz, 1H), 8.07 (s, 1H), 7.61 (d, J = 2.4 Hz, 1H), 6.89 (d, J = 8.3 Hz, 1H), 6.67 (d, J = 8.2 Hz, 1H), 6.00 (d, J = |
| | | 2.3 Hz, 1H), 4.69 (s, 2H), 3.19 (d, J = 12.5 |
| | | Hz, 2H), 3.01 (d, J = 6.3 Hz, 2H), 2.74 (td, |
| | | J = 12.7, 2.9 Hz, 2H), 2.03 (s, 3H), 1.95 (s, |
| | | 3H), 1.85-1.75 (m, 2H), 1.74-1.63 (m, |
| | | 1H), 1.32-1.15 (m, 2H). |
| BAA- 227 | 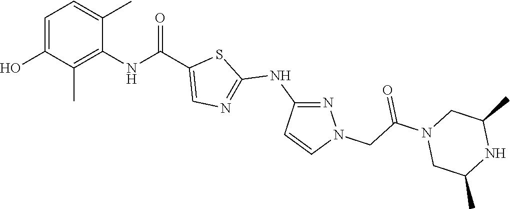 [SMILES: Cc1ccc(O)c(C)c1NC(=O)c1cnc(Nc2ccn(CC(=O)N3C[C@@H](C)N[C@@H](C)C3)n2)s1] | MS (ES+) m/z 484.0 (M + H). ¹H NMR (300 MHz, Methanol-d₄) δ 8.28 (s, 1H), 7.74 (s, 1H), 6.96 (d, J = 8.2 Hz, 1H), 6.73 (d, J = 8.2 Hz, 1H), 6.22 (s, 1H), 5.48- 5.15 (m, 2H), 4.69 (d, J = 13.2 Hz, 1H), 4.32 (d, J = 13.8 Hz, 1H), 3.63-3.48 (m, 1H), 2.83-2.69 (m, 1H), 2.18 (s, 3H), 2.12 (s, 3H), 1.45-1.35 (m, 6H). |
| | 2-((1-(2-((3R,5S)-3,5-dimethylpiperazin-1- | |
| | yl)-2-oxoethyl)-1H-pyrazol-3-yl)amino)-N- | |
| | (3-hydroxy-2,6-dimethylphenyl)thiazole-5- | |
| | carboxamide | |
| BAA- 228 | 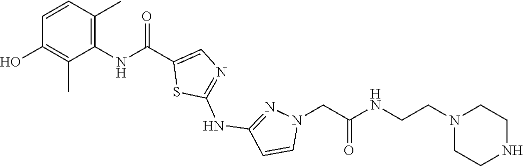 [SMILES: Cc1ccc(O)c(C)c1NC(=O)c1cnc(Nc2ccn(CC(=O)NCCN3CCNCC3)n2)s1] N-(3-hydroxy-2,6-dimethylphenyl)-2-((1- (2-oxo-2-((2-(piperazin-1- yl)ethyl)amino)ethyl)-1H-pyrazol-3- yl)amino)thiazole-5-carboxamide | MS (ES+) m/z 499.0 (M + H). ¹H NMR (300 MHz, DMSO-d₆) δ 11.77- 11.37 (br s, 1H), 11.30-11.10 (br s, 1H), 9.59 (s, 1H), 9.55 (s, 2H), 9.42-9.02 (br s, 1H), 8.42 (t, J = 5.6 Hz, 1H), 8.15 (s, 1H), 7.71 (d, J = 2.4 Hz, 1H), 6.88 (d, J = 8.2 Hz, 1H), 6.70 (d, J = 8.2 Hz, 1H), 6.01 (d, J = 2.4 Hz, 1H), 4.81 (s, 2H), 3.81-3.61 (br s, 2H), 3.58-3.43 (m, 6H), 3.35-3.12 (m, 4H), 2.07 (s, 3H), 1.98 (s, 3H). ¹H NMR (300 MHz, DMSO-d₆ + D₂O) δ 8.09 (s, 1H), 7.65 (d, J = 2.4 Hz, 1H), 6.89 (d, J = 8.3 Hz, 1H), 6.67 (d, J = 8.2 Hz, 1H), 6.03 (d, J = 2.4 Hz, 1H), 4.77 (s, 2H), 3.48 (t, J = 6.1 Hz, 2H), 3.45-3.27 (m, 8H), 3.20 (t, J = 6.1 Hz, 2H), 2.04 (s, 3H), 1.95 (s, 3H). |
| BAA- 229 | 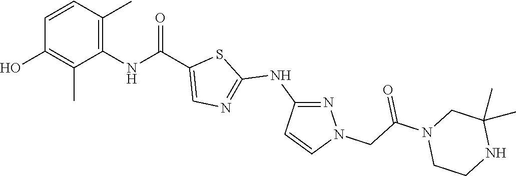 [SMILES: Cc1ccc(O)c(C)c1NC(=O)c1cnc(Nc2ccn(CC(=O)N3CCNC(C)(C)C3)n2)s1] 2-((1-(2-(3,3-dimethylpiperazin-1-yl)-2- oxoethyl)-1H-pyrazol-3-yl)amino)-N-(3- hydroxy-2,6-dimethylphenyl)thiazole-5- carboxamide | MS (ES+) m/z 484.1 (M + H). ¹H NMR (300 MHz, DMSO-d₆) δ 11.42- 11.02 (br s, 1H), 9.59 (s, 1H), 9.45 (s, 2H), 9.35-8.95 (br s, 1H), 8.15 (s, 1H), 7.63 (dd, J = 11.3, 2.4 Hz, 1H), 6.88 (d, J = 8.2 Hz, 1H), 6.70 (d, J = 8.2 Hz, 1H), 6.02 (dd, J = 4.7, 2.3 Hz, 1H), 5.22 (s, 1H), 5.06 (s, 1H), 3.78-3.62 (m, 3H), 3.51 (s, 1H), 3.25-3.08 (m, 2H), 2.07 (s, 3H), 1.98 (s, 3H), 1.37 (s, 3H), 1.29 (s, 3H). ¹H NMR (300 MHz, DMSO-d₆ + D₂O) δ 8.09 (s, 1H), 7.61 (dd, J = 6.3, 2.4 Hz, 1H), 6.88 (d, J = 8.2 Hz, 1H), 6.67 (d, J = 8.2 Hz, 1H), 6.03 (dd, J = 5.2, 2.3 Hz, 1H), 5.16 (s, 1H), 5.02 (s, 1H), 3.75-3.57 (m, 3H), 3.48 (s, 1H), 3.25-3.07 (m, 2H), 2.04 (s, 3H), 1.95 (s, 3H), 1.33 (s, 3H), 1.25 (s, 3H). |
| BAA- 230 | 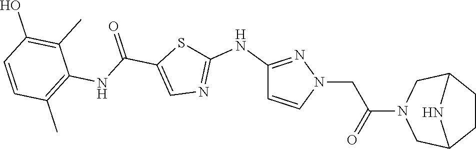 [SMILES: Cc1ccc(O)c(C)c1NC(=O)c1cnc(Nc2ccn(CC(=O)N3CC4CCC(C3)N4)n2)s1] 2-((1-(2-(3,8-diazabicyclo[3.2.1]octan-3- yl)-2-oxoethyl)-1H-pyrazol-3-yl)amino)-N- (3-hydroxy-2,6-dimethylphenyl)thiazole-5- carboxamide | MS (ES+) m/z 482.0 (M + H). ¹H NMR (300 MHz, DMSO-d₆) δ 11.42-11.02 (br s, 1H), 9.60 (s, 1H), 9.57-8.93 (m, 3H), 8.15 (s, 1H), 7.64 (d, J = 2.4 Hz, 1H), 6.88 (d, J = 8.2 Hz, 1H), 6.70 (d, J = 8.2 Hz, 1H), 6.02 (d, J = 2.3 Hz, 1H), 5.28 (d, J = 16.7 Hz, 1H), 5.00 (d, J = 16.5 Hz, 1H), 4.13-4.01 (m, 3H), 3.90 (d, J = 13.7 Hz, 1H), 3.62 (d, J = 13.5 Hz, 1H), 3.12 (d, J = 13.8 Hz, 1H), 2.07 (s, 3H), 1.98 (s, 3H), 1.98-1.86 (m, 3H), 1.70-1.59 (m, 1H). ¹H NMR (300 MHz, DMSO-d₆ + D₂O) δ 8.08 (s, 1H), 7.61 (d, J = 2.4 Hz, 1H), 6.88 (d, J = 8.3 Hz, 1H), 6.67 (d, J = 8.2 Hz, 1H), 6.03 (d, J = 2.4 Hz, 1H), 5.21 (d, J = 16.6 Hz, 1H), 4.95 (d, J = |
| | | 16.5 Hz, 1H), 4.14 (d, J = 14.1 Hz, |
| | | 1H), 4.02 (s, 2H), 3.54 (d, J = 13.6 |
| | | Hz, 1H), 3.04 (d, J = 14.0 Hz, 1H), |
| | | 2.04 (s, 3H), 1.95 (s, 6H), 1.71-1.59 (m, 1H). |

Ester Formation from BAA-059

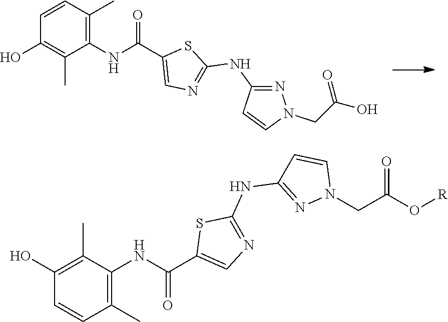

### Isopropyl 2-[3-[[5-[(3-hydroxy-2,6-dimethyl-phenyl) carbamoyl]thiazol-2-yl]amino]pyrazol-1-yl]acetate (BAA-209)

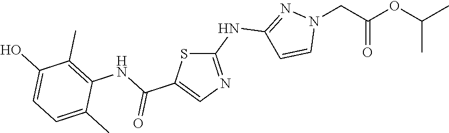 [SMILES: Cc1ccc(O)c(C)c1NC(=O)c1cnc(Nc2ccn(CC(=O)OC(C)C)n2)s1]

To a solution of 2-[3-[5-[(3-hydroxy-2,6-dimethyl-phenyl) carbamoyl]thiazol-2-yl]amino]pyrazol-1-yl]acetic acid (BAA-059)(35 mg, 0.0903 mmol, 1.0 eq) in IPA (3 mL), was added sulfuric acid (95% in water, 0.00051 mL, 0.00903 mmol, 0.1 eq) and the reaction mixture was stirred at RT for 18 h. The mixture was concentrated in vacuo and partitioned between EtOAc and water. The aqueous phase was extracted with EtOAc (x 3), and the combined organic layers dried (MgSO₄), filtered and concentrated under reduced pressure. The crude material was chromatographed (SiO₂) eluting with 0-20% MeOH: DCM to afford the title compound (15 mg, 0.0349 mmol, 39%) as a white solid. MS (ES+) m/z 429.9 (M+H). ¹H NMR (300 MHz, DMSO-d₆) δ 11.09 (s, 1H), 9.52 (s, 1H), 9.19 (s, 1H), 8.10 (s, 1H), 7.69 (d, J=2.4 Hz, 1H), 6.88 (d, J=8.2 Hz, 1H), 6.68 (d, J=8.2 Hz, 1H), 6.03 (d, J=2.4 Hz, 1H), 5.05-4.86 (m, 3H), 2.07 (s, 3H), 1.98 (s, 3H), 1.22 (d, J=6.3 Hz, 6H).

General Method U. Synthesis of Phenol-Amides from Unsubstituted or Substituted 2-(3-((5-((3-Methoxy-2,6-Dimethylphenyl)Carbamoyl)Thiazol-2-Yl)Amino)-1H-Pyrazol-1-Yl) Acetic Acid Coupling with Amines and Methoxy Deprotection

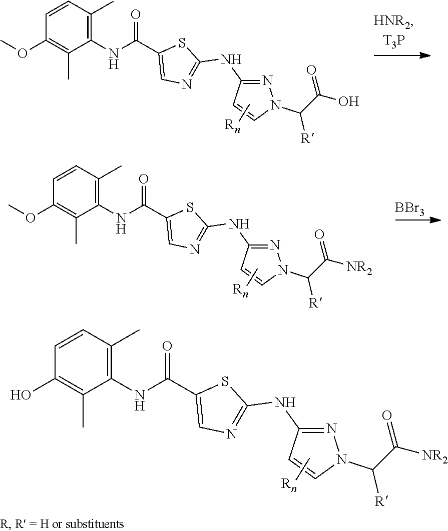

To a solution of the carboxylic acid (1 eq), amine (2 eq) and DIPEA (3 eq) in THF was added 1-propanephosphonic anhydride (2 eq), the reaction was heated to 50° C. and stirred for 24-96 h. The reaction was cooled to room temperature and diluted with saturated aqueous NaHCO₃. The suspension was filtered, and washed with water (10 mL). The residue was taken up in DCM, filtered and concentrated under reduced pressure to afford the desired amide.

The methoxy protected intermediate was then suspended in DCM and BBr₃ (8-9 eq) were added. The resulting mixture was stirred at rt, under nitrogen for 16 h. The reaction was quenched by addition of MeOH(5 mL) then concentrated under reduced pressure. The crude was purified by reverse phase chromatography with a gradient of MeCN/water to afford the desired phenol.

### N-(3-Hydroxy-2.6-dimethylphenyl)-2-((1-(1-oxo-1-(pyrrolidin-1-yl)propan-2-yl)-1H-pyrazol-3-yl)amino)thiazole-5-carboxamide( ) BAA-506

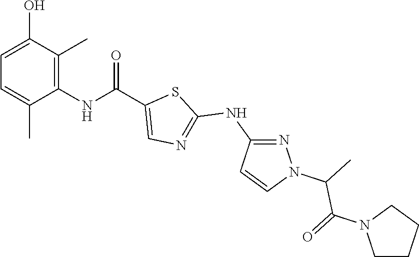 [SMILES: Cc1ccc(O)c(C)c1NC(=O)c1cnc(Nc2ccn(C(C)C(=O)N3CCCC3)n2)s1]

Prepared as described in method U from 2-[3-[5-[(3-methoxy-2,6-dimethyl-phenyl) carbamoyl]thiazol-2-yl]amino]pyrazol-1-yl]propanoic acid (100 mg, 0.241 mmol), pyrrolidine (0.040 mL, 0.481 mmol), DIPEA (0.13 mL, 0.722 mmol) and 1-propanephosphonic anhydride (0.14 mL, 0.481 mmol) in THF (2.41 mL) to afford the intermediate amide (57 mg, 0.0675 mmol, 28.05% yield) as a beige solid. MS (ES+) m/z=469.3 [M+H]+. This intermediate was treated with BBr₃ (0.54 mL, 0.54 mmol) to afford the title compound (23 mg, 0.0501 mmol, 74.19% yield) as a white solid after HPLC purification (C18, ammonium formate in MeCN/H2O). MS (ES+) m/z=455.4 [M+H]⁺, ¹H NMR (400 MHz, DMSO-D6) δ 11.03 (s, 1H), 9.49 (s, 1H), 9.16 (s, 1H), 8.09 (s, 1H), 7.71 (d, J=2.4 Hz, 1H), 6.88 (d, J=8.2 Hz, 1H), 6.68 (d, J=8.2 Hz, 1H), 6.00 (d, J=2.4 Hz, 1H), 5.24 (q, J=6.8 Hz, 1H), 3.67-3.45 (m, 2H), 3.29 (dd, J=6.9, 2.3 Hz, 2H), 2.06 (s, 3H), 1.98 (s, 3H), 1.95-1.81 (m, J=5.7 Hz, 2H), 1.76 (p, J=6.7 Hz, 2H), 1.55 (d, J=6.9 Hz, 3H).

The following example compounds were prepared similarly using Method U with appropriately substituted carboxylic acids and amines.

| # | Structure & Name | Analytical Data |
|---|---|---|
| BAA- 488 | 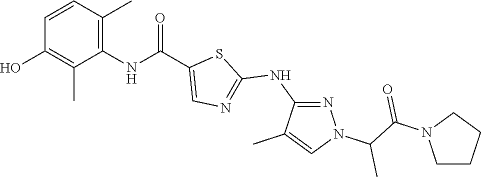 [SMILES: Cc1cn(C(C)C(=O)N2CCCC2)nc1Nc1ncc(C(=O)Nc2c(C)ccc(O)c2C)s1] | ¹H NMR (400 MHz, DMSO-d₆) δ 10.62 (s, 1H), 9.42 (s, 1H), 9.12 (s, 1H), 8.06 (s, 1H), 7.49 (d, J = 1.0 Hz, 1H), 6.84 (d, J = 8.2 Hz, 1H), 6.64 (d, J = 8.2 Hz, 1H), 5.14 (q, J = 6.9 Hz, 1H), 3.62-3.45 (m, 2H), 3.27-3.21 (m, 1H), 2.02 (s, 3H), 1.99-1.92 (m, 6H), 1.90-1.77 (m, 2H), 1.76-1.66 (m, 2H), 1.48 (d, J = 6.8 Hz, 3H). MS (ES⁺) m/z 469.4 (M + H)⁺. |
| | N-(3-hydroxy-2,6-dimethylphenyl)-2- | |
| | ((4-methyl-1-(1-oxo-1-(pyrrolidin-1- | |
| | yl)propan-2-yl)-1H-pyrazol-3- | |
| | yl)amino)thiazole-5-carboxamide | |
| BAA- 489 | 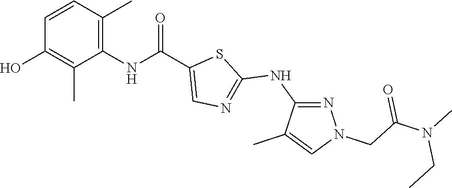 [SMILES: CCN(C)C(=O)Cn1cc(C)c(Nc2ncc(C(=O)Nc3c(C)ccc(O)c3C)s2)n1] | ¹H NMR (400 MHz, DMSO-d₆) δ 9.46 (s, 1H), 9.20 (s, 1H), 8.09 (s, 1H), 7.40 (d, J = 9.5 Hz, 1H), 6.87 (d, J = 8.2 Hz, 1H), 6.67 (d, J = 8.1 Hz, 1H), 4.94 (d, J = 5.8 Hz, 2H), 3.43-3.39 (m, 1H), 3.31-3.28 (m, 1H), 3.00 (s, 2H), 2.81 (s, 1H), 2.06 (s, 3H), 2.00 (s, 3H), 1.98 (s, 3H), 1.14 (t, J = 7.1 Hz, 1H), 1.01 (t, J = 7.1 Hz, 2H). MS (ES⁺) m/z 443.4 (M + H)⁺. |
| | 2-((1-(2-(ethyl(methyl)amino)-2- | |
| | oxoethyl)-4-methyl-1H-pyrazol-3- | |
| | yl)amino)-N-(3-hydroxy-2,6- | |
| | dimethylphenyl)thiazole-5- | |
| | carboxamide | |
| BAA- 491 | 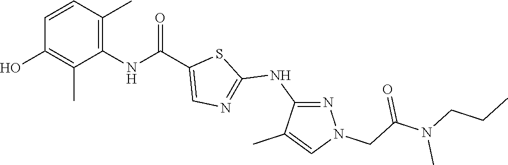 [SMILES: CCCN(C)C(=O)Cn1cc(C)c(Nc2ncc(C(=O)Nc3c(C)ccc(O)c3C)s2)n1] | ¹H NMR (400 MHz, DMSO-d₆) δ 10.63 (s, 1H), 9.45 (s, 1H), 9.17 (s, 1H), 8.09 (s, 1H), 7.40 (dd, J = 12.6, 1.0 Hz, 1H), 6.87 (d, J = 8.2 Hz, 1H), 6.67 (d, J = 8.1 Hz, 1H), 4.95 (d, J = 4.4 Hz, 2H), 3.23 (dd, J = 8.1, 6.6 Hz, 1H), 3.01 (s, 2H), 2.81 (s, 1H), 2.06 (s, 3H), 2.00 (s, 3H), 1.98 (s, 3H), 1.57 (p, J = 7.5 Hz, 1H), 1.49-1.39 (m, 1H), 0.89 (t, J = 7.3 Hz, 1H), 0.80 (t, J = 7.4 Hz, 2H). MS (ES⁺) m/z 457.2 (M + H)⁺. |
| | N-(3-hydroxy-2,6-dimethylphenyl)-2- | |
| | ((4-methyl-1-(2-(methyl(propyl)amino)- | |
| | 2-oxoethyl)-1H-pyrazol-3- | |
| | yl)amino)thiazole-5-carboxamide | |
| BAA- 492 | 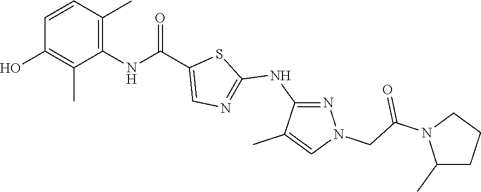 [SMILES: Cc1cn(CC(=O)N2CCCC2C)nc1Nc1ncc(C(=O)Nc2c(C)ccc(O)c2C)s1] | ¹H NMR (400 MHz, DMSO-d₆) δ 9.46 (s, J = 2.1 Hz, 1H), 9.19 (s, 1H), 8.09 (s, 1H), 7.46-7.38 (m, 1H), 6.87 (d, J = 8.2 Hz, 1H), 6.67 (d, J = 8.1 Hz, 1H), 4.98- 4.77 (m, 2H), 4.31-3.90 (m, 1H), 3.58 (dq, J = 9.7, 3.5 Hz, 1H), 3.50 (dt, J = 9.7, 7.5 Hz, 1H), 2.06 (s, 3H), 1.99 (d, J = 10.4 Hz, 6H), 1.95-1.79 (m, 2H), 1.71- 1.46 (m, 1H), 1.20 (d, J = 6.4 Hz, 1H), 1.09 (d, J = 6.3 Hz, 2H). MS (ES⁺) m/z 469.2 (M + H)⁺. |
| | N-(3-hydroxy-2,6-dimethylphenyl)-2- | |
| | ((4-methyl-1-(2-(2-methylpyrrolidin-1- | |
| | yl)-2-oxoethyl)-1H-pyrazol-3- | |
| | yl)amino)thiazole-5-carboxamide | |
| BAA- 493 | 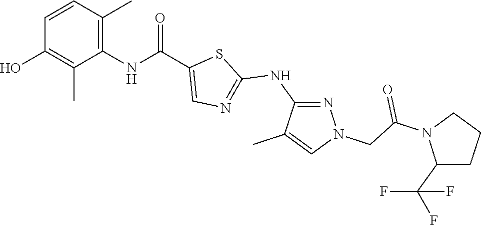 [SMILES: Cc1cn(CC(=O)N2CCCC2C(F)(F)F)nc1Nc1ncc(C(=O)Nc2c(C)ccc(O)c2C)s1] | ¹H NMR (400 MHz, DMSO-d₆) δ 9.46 (s, 1H), 9.18 (s, 1H), 8.10 (s, 1H), 7.47- 7.37 (m, 1H), 6.87 (d, J = 8.2 Hz, 1H), 6.68 (d, J = 8.2 Hz, 1H), 5.22-4.88 (m, 2H), 4.84-4.66 (m, 1H), 3.80-3.70 (m, 1H), 3.69-3.61 (m, 1H), 2.32-1.80 (m, 14H). MS (ES⁺) m/z 523.1 (M + H)⁺. |
| | N-(3-hydroxy-2,6-dimethylphenyl)-2- | |
| | ((4-methyl-1-(2-oxo-2-(2- | |
| | (trifluoromethyl)pyrrolidin-1-yl)ethyl)- | |
| | 1H-pyrazol-3-yl)amino)thiazole-5- | |
| | carboxamide | |
| BAA- 497 | 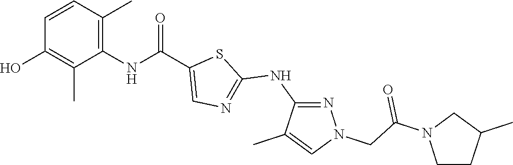 [SMILES: Cc1cn(CC(=O)N2CCC(C)C2)nc1Nc1ncc(C(=O)Nc2c(C)ccc(O)c2C)s1] | ¹H NMR (400 MHz, DMSO-d₆) δ 10.63 (s, 1H), 9.46 (s, 1H), 9.18 (s, 1H), 8.10 (s, 1H), 7.40 (s, 1H), 6.87 (d, J = 8.2 Hz, 1H), 6.68 (d, J = 8.2 Hz, 1H), 4.92-4.76 (m, 2H), 3.79-3.61 (m, 1H), 3.56-3.44 (m, 1H), 3.30-3.19 (m, 1H), 3.17 (s, 0H), 3.10-2.76 (m, 1H), 2.35-2.11 (m, 1H), 2.07-1.85 (m, 10H), 1.62-1.34 (m, 1H), 1.05-0.94 (m, 3H). MS (ES⁺) m/z 469.2 (M + H)⁺. |
| | N-(3-hydroxy-2,6-dimethylphenyl)-2- | |
| | ((4-methyl-1-(2-(3-methylpyrrolidin-1- | |
| | yl)-2-oxoethyl)-1H-pyrazol-3- | |
| | yl)amino)thiazole-5-carboxamide | |
| BAA- 500 | 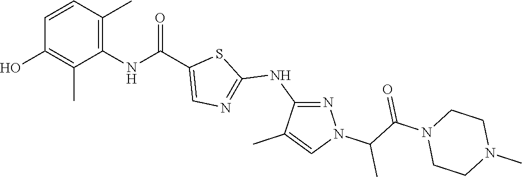 [SMILES: Cc1cn(C(C)C(=O)N2CCN(C)CC2)nc1Nc1ncc(C(=O)Nc2c(C)ccc(O)c2C)s1] | ¹H NMR (400 MHz, DMSO-d₆) δ 9.38 (s, 1H), 9.08 (s, 1H), 8.19 (s, 1H), 8.05 (s, 1H), 7.46 (d, J = 1.0 Hz, 1H), 6.84 (d, J = 8.2 Hz, 1H), 6.64 (d, J = 8.2 Hz, 1H), 5.39 (q, J = 6.9 Hz, 1H), 3.48 (d, J = 23.7 Hz, 3H), 2.09 (s, 6H), 2.03 (d, J = 1.3 Hz, 3H), 1.96 (d, J = 5.2 Hz, 6H), 1.46 (d, J = 6.9 Hz, 3H). MS (ES⁺) m/z 498.3 (M + H)⁺. |
| | N-(3-hydroxy-2,6-dimethylphenyl)-2- | |
| | ((4-methyl-1-(1-(4-methylpiperazin-1- | |
| | yl)-1-oxopropan-2-yl)-1H-pyrazol-3- | |
| | yl)amino)thiazole-5-carboxamide | |
| BAA- 502 | 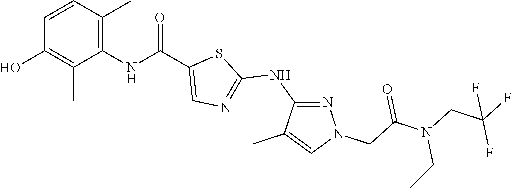 [SMILES: CCN(CC(F)(F)F)C(=O)Cn1cc(C)c(Nc2ncc(C(=O)Nc3c(C)ccc(O)c3C)s2)n1] | ¹H NMR (400 MHz, DMSO-d₆) δ 10.67 (s, 1H), 9.45 (s, 1H), 9.18 (s, 1H), 8.09 (s, 1H), 7.44 (d, J = 2.3 Hz, 1H), 6.87 (d, J = 8.2 Hz, 1H), 6.67 (d, J = 8.2 Hz, 1H), 5.10 (s, 1H), 5.00 (s, 1H), 4.41 (q, J = 9.2 Hz, 1H), 4.16 (q, J = 9.6 Hz, 1H), 3.54 (q, J = 7.1 Hz, 1H), 3.40-3.36 (m, 1H), 2.06 (s, 3H), 2.01 (s, 3H), 1.98 (s, 3H), 1.21 (t, J = 7.1 Hz, 2H), 1.05 (t, J = 7.0 Hz, 1H). MS (ES⁺) m/z 511.2 (M + H)⁺. |
| | 2-((1-(2-(ethyl(2,2,2- | |
| | trifluoroethyl)amino)-2-oxoethyl)-4- | |
| | methyl-1H-pyrazol-3-yl)amino)-N-(3- | |
| | hydroxy-2,6-dimethylphenyl)thiazole- | |
| | 5-carboxamide | |
| BAA- 504 | 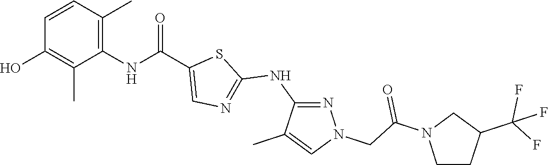 [SMILES: Cc1cn(CC(=O)N2CCC(C(F)(F)F)C2)nc1Nc1ncc(C(=O)Nc2c(C)ccc(O)c2C)s1] | 1H NMR (400 MHz, DMSO-d₆) δ 9.46 (s, 1H), 8.17 (s, 1H), 8.10 (d, J = 1.1 Hz, 1H), 7.40 (dd, J = 6.1, 1.0 Hz, 1H), 6.87 (d, J = 8.2 Hz, 1H), 6.68 (d, J = 8.2 ^{Hz}, 1H), 4.98-4.85 (m, 2H), 3.97-3.20 (m, 5H), 2.32-1.88 (m, 11H). MS (ES⁺) m/z 523.1 (M + H)⁺. |
| | N-(3-hydroxy-2,6-dimethylphenyl)-2- | |
| | ((4-methyl-1-(2-oxo-2-(3- | |
| | (trifluoromethyl)pyrrolidin-1-yl)ethyl)- | |
| | 1H-pyrazol-3-yl)amino)thiazole-5- | |
| | carboxamide | |
| BAA- 507 | 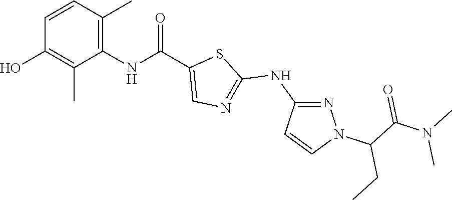 [SMILES: CCC(C(=O)N(C)C)n1ccc(Nc2ncc(C(=O)Nc3c(C)ccc(O)c3C)s2)n1] | ¹H NMR (400 MHz, DMSO-d₆) δ 11.04 (s, 1H), 9.49 (s, 1H), 9.16 (s, 1H), 8.10 (s, 1H), 7.70 (d, J = 2.4 Hz, 1H), 6.88 (d, J = 8.2 Hz, 1H), 6.68 (d, J = 8.2 Hz, 1H), 6.02 (d, J = 2.4 Hz, 1H), 5.24 (t, J = 7.4 Hz, 1H), 3.07 (s, 3H), 2.84 (s, 3H), 2.06 (s, 3H), 2.02-1.93 (m, 5H), 0.82 (t, J = 7.3 Hz, 3H). MS (ES⁺) m/z 443.4 (M + H)⁺. |
| | 2-((1-(1-(dimethylamino)-1-oxobutan- | |
| | 2-yl)-1H-pyrazol-3-yl)amino)-N-(3- | |
| | hydroxy-2,6-dimethylphenyl)thiazole- | |
| | 5-carboxamide | |
| BAA- 508 | 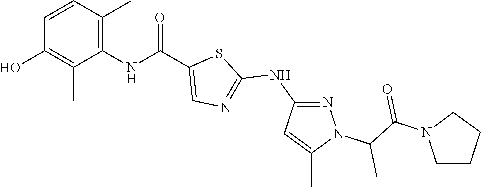 [SMILES: Cc1ccc(O)c(C)c1NC(=O)c1cnc(Nc2cc(C)n(C(C)C(=O)N3CCCC3)n2)s1] | ¹H NMR (400 MHz, DMSO-d₆) δ 10.92 (s, 1H), 9.46 (s, 1H), 9.16 (s, 1H), 8.07 (s, 1H), 6.87 (d, J = 8.2 Hz, 1H), 6.67 (d, J = 8.2 Hz, 1H), 5.85 (d, J = 0.8 Hz, 1H), 5.19 (q, J = 6.8 Hz, 1H), 3.45-3.35 (m, 1H), 2.79-2.63 (m, 1H), 2.27-2.18 (m, 3H), 2.06 (s, 3H), 1.98 (s, 3H), 1.85- 1.59 (m, 4H), 1.48 (d, J = 6.8 Hz, 3H). MS (ES⁺) m/z 469.4 (M + H)⁺. |
| | N-(3-hydroxy-2,6-dimethylphenyl)-2- | |
| | ((5-methyl-1-(1-oxo-1-(pyrrolidin-1- | |
| | yl)propan-2-yl)-1H-pyrazol-3- | |
| | yl)amino)thiazole-5-carboxamide | |
| BAA- 509 | 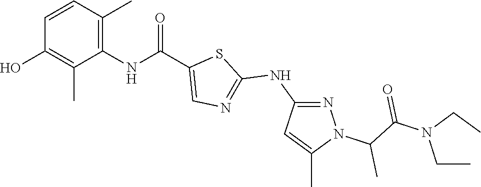 [SMILES: CCN(CC)C(=O)C(C)n1nc(Nc2ncc(C(=O)Nc3c(C)ccc(O)c3C)s2)cc1C] | ¹H NMR (400 MHz, DMSO-d₆) δ 10.92 (s, 1H), 9.46 (s, 1H), 9.16 (s, 1H), 8.07 (s, 1H), 6.87 (d, J = 8.2 Hz, 1H), 6.67 (d, J = 8.2 Hz, 1H), 5.85 (d, J = 0.8 Hz, 1H), 5.19 (q, J = 6.8 Hz, 1H), 3.45-3.35 (m, 1H), 2.79-2.63 (m, 1H), 2.27-2.18 (m, 3H), 2.06 (s, 3H), 1.98 (s, 3H), 1.85- 1.59 (m, 4H), 1.48 (d, J = 6.8 Hz, 3H). MS (ES⁺) m/z 471.4 (M + H)⁺. |
| | 2-((1-(1-(diethylamino)-1-oxopropan- | |
| | 2-yl)-5-methyl-1H-pyrazol-3-yl)amino)- | |
| | N-(3-hydroxy-2,6-dimethylphenyl) | |
| | thiazole-5-carboxamide | |
| BAA- 510 | 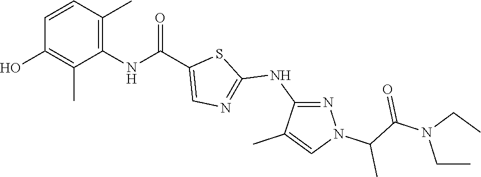 | ¹H NMR (400 MHz, MeOD) δ 8.03 (s, 1H), 6.92 (d, J = 8.2 Hz, 1H), 6.69 (d, J = 8.2 Hz, 1H), 5.87 (d, J = 0.8 Hz, 1H), 5.43 (q, J = 6.7 Hz, 1H), 4.04-3.42 (m, 1H), 2.88 (s, 2H), 2.53 (s, 3H), 2.34 (d, J = 0.7 Hz, 3H), 2.16 (s, 3H), 2.11 (s, 3H), 1.59 (d, J = 6.7 Hz, 3H), 1.29 (s, 2H). MS (ES⁺) m/z 498.4 (M + H)⁺. |
| | 2-((1-(1-(diethylamino)-1-oxopropan- | |
| | 2-yl)-4-methyl-1H-pyrazol-3-yl)amino)- | |
| | N-(3-hydroxy-2,6-dimethylphenyl) | |
| | thiazole-5-carboxamide | |
| BAA- 511 | 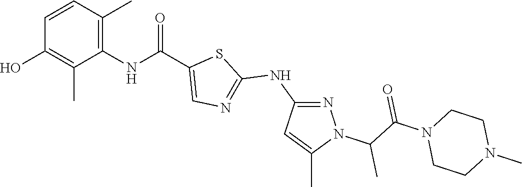 [SMILES: Cc1ccc(O)c(C)c1NC(=O)c1cnc(Nc2cc(C)n(C(C)C(=O)N3CCN(C)CC3)n2)s1] | ¹H NMR (400 MHz, MeOD) δ 8.03 (s, 1H), 6.92 (d, J = 8.2 Hz, 1H), 6.69 (d, J = 8.2 Hz, 1H), 5.87 (d, J = 0.8 Hz, 1H), 5.43 (q, J = 6.7 Hz, 1H), 4.04-3.42 (m, 1H), 2.88 (s, 2H), 2.53 (s, 3H), 2.34 (d, J = 0.7 Hz, 3H), 2.16 (s, 3H), 2.11 (s, 3H), 1.59 (d, J = 6.7 Hz, 3H), 1.29 (s, 2H). MS (ES⁺) m/z 498.4 (M + H)⁺. |
| | N-(3-hydroxy-2,6-dimethylphenyl)-2- | |
| | ((5-methyl-1-(1-(4-methylpiperazin-1- | |
| | yl)-1-oxopropan-2-yl)-1H-pyrazol-3- | |
| | yl)amino)thiazole-5-carboxamide | |
| BAA- 512 | 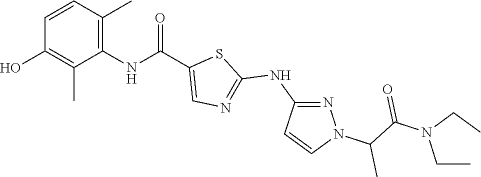 [SMILES: CCN(CC)C(=O)C(C)n1ccc(Nc2ncc(C(=O)Nc3c(C)ccc(O)c3C)s2)n1] | ¹H NMR (400 MHz, DMSO-d₆) δ 11.04 (s, 1H), 9.48 (s, 1H), 9.15 (s, 1H), 8.09 (s, 1H), 7.70 (d, J = 2.4 Hz, 1H), 6.88 (d, J = 8.2 Hz, 1H), 6.68 (d, J = 8.2 Hz, 1H), 6.00 (d, J = 2.4 Hz, 1H), 5.37 (q, J = 6.8 Hz, 1H), 3.54-3.40 (m, 1H), 3.32-3.19 (m, 2H), 2.06 (s, 3H), 1.98 (s, 3H), 1.53 (d, J = 6.8 Hz, 3H), 1.09 (t, J = 7.1 Hz, 3H), 1.01 (t, J = 7.0 Hz, 3H). MS (ES⁺) m/z 457.4 (M + H)⁺. |
| | 2-((1-(1-(diethylamino)-1-oxopropan- | |
| | 2-yl)-1H-pyrazol-3-yl)amino)-N-(3- | |
| | hydroxy-2,6-dimethylphenyl)thiazole- | |
| | 5-carboxamide | |
| BAA- 514 | 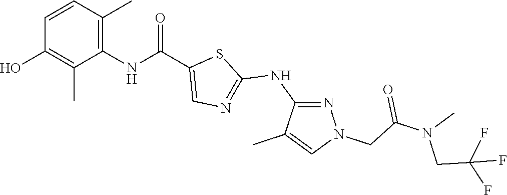 [SMILES: Cc1cn(CC(=O)N(C)CC(F)(F)F)nc1Nc1ncc(C(=O)Nc2c(C)ccc(O)c2C)s1] | ¹H NMR (400 MHz, DMSO-d₆) δ 10.63 (s, 1H), 9.46 (s, 1H), 9.17 (s, 1H), 8.10 (s, 1H), 7.46-7.34 (m, 1H), 6.87 (d, J = 8.2 Hz, 1H), 6.67 (d, J = 8.1 Hz, 1H), 5.19-4.97 (m, 2H), 4.30 (dq, J = 107.4, 9.4 Hz, 2H), 3.19-2.92 (m, 3H), 2.06 (s, 3H), 2.01 (s, 3H), 1.98 (s, 3H). MS (ES⁺) m/z 497.1 (M + H)⁺. |
| | N-(3-hydroxy-2,6-dimethylphenyl)-2- | |
| | ((4-methyl-1-(2-(methyl(2,2,2- | |
| | trifluoroethyl)amino)-2-oxoethyl)-1H- | |
| | pyrazol-3-yl)amino)thiazole-5- | |
| | carboxamide | |
| BAA- 522 | 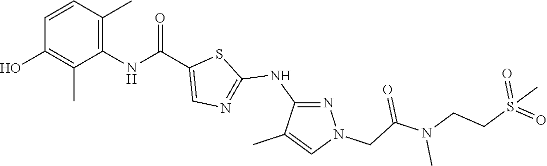 [SMILES: Cc1cn(CC(=O)N(C)CCS(C)(=O)=O)nc1Nc1ncc(C(=O)Nc2c(C)ccc(O)c2C)s1] | ¹H NMR (400 MHz, DMSO-d₆) δ 10.68 (s, 1H), 9.46 (d, J = 2.6 Hz, 1H), 9.16 (s, 1H), 8.10 (s, 1H), 7.37 (dd, J = 3.8, 0.9 Hz, 1H), 6.87 (d, J = 8.2 Hz, 1H), 6.67 (d, J = 8.2 Hz, 1H), 5.10-4.93 (m, 2H), 3.83- 3.66 (m, 2H), 3.62-3.33 (m, 2H), 3.11- 3.05 (m, 3H), 3.01-2.83 (m, 3H), 2.06 (s, 3H), 2.02-1.91 (m, 6H). MS (ES⁺) m/z 521.2 (M + H)⁺. |
| | N-(3-hydroxy-2,6-dimethylphenyl)-2- | |
| | ((4-methyl-1-(2-(methyl(2- | |
| | (methylsulfonyl)ethyl)amino)-2- | |
| | oxoethyl)-1H-pyrazol-3-yl)amino) | |
| | thiazole-5-carboxamide | |
| BAA- 523 | 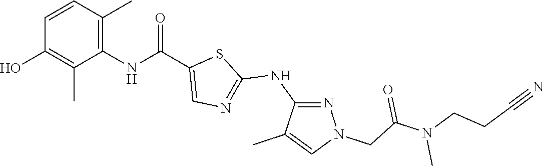 [SMILES: Cc1cn(CC(=O)N(C)CCC#N)nc1Nc1ncc(C(=O)Nc2c(C)ccc(O)c2C)s1] | ¹H NMR (400 MHz, DMSO-d₆) δ 9.45 (s, 1H), 9.24 (s, 1H), 8.09 (s, 1H), 7.43- 7.35 (m, 1H), 6.87 (d, J = 8.2 Hz, 1H), 6.68 (d, J = 8.2 Hz, 1H), 5.11-4.88 (m, 2H), 3.75-3.52 (m, 2H), 3.13-2.83 (m, 3H), 2.92-2.67 (m, 2H), 2.06 (s, 3H), 2.02-1.91 (m, 6H). MS (ES⁺) m/z 468.2 (M + H)⁺. |
| | 2-((1-(2-((2- | |
| | cyanoethyl)(methyl)amino)-2-oxo- | |
| | ethyl)-4-methyl-1H-pyrazol-3-yl) | |
| | amino)-N-(3-hydroxy-2,6-dimethyl | |
| | phenyl)thiazole-5-carboxamide | |
| BAA- 532 | 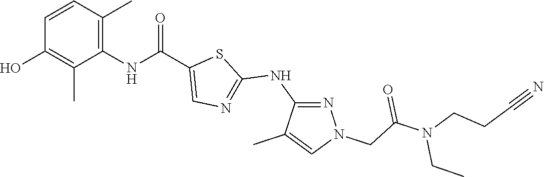 [SMILES: CCN(CCC#N)C(=O)Cn1cc(C)c(Nc2ncc(C(=O)Nc3c(C)ccc(O)c3C)s2)n1] | ¹H NMR (400 MHz, DMSO-d₆) δ 9.46 (s, 1H), 8.38 (s, 1H), 8.09 (s, 1H), 7.44- 7.34 (m, 1H), 6.87 (d, J = 8.2 Hz, 1H), 6.68 (d, J = 8.2 Hz, 1H), 5.06-4.94 (m, 2H), 3.72-3.67 (m, 1H), 3.52-3.45 (m, 2H), 3.35-3.28 (m, 1H), 2.89-2.68 (m, 2H), 2.06 (s, 3H), 2.02-1.99 (m, 3H), 1.98 (s, 3H), 1.19-1.00 (m, 3H). MS (ES⁺) m/z 482.2 (M + H)⁺. |
| | 2-((1-(2-((2-cyanoethyl)(ethyl)amino)- | |
| | 2-oxoethyl)-4-methyl-1H-pyrazol-3- | |
| | yl)amino)-N-(3-hydroxy-2,6- | |
| | dimethylphenyl)thiazole-5- | |
| | carboxamide | |
| BAA- 533 | 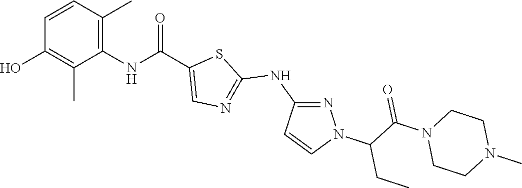 [SMILES: CCC(C(=O)N1CCN(C)CC1)n1ccc(Nc2ncc(C(=O)Nc3c(C)ccc(O)c3C)s2)n1] | ¹H NMR (400 MHz, DMSO-d₆) δ 8.44 (s, 4H), 8.04 (s, 1H), 7.63 (d, J = 2.5 Hz, 1H), 6.92 (d, J = 8.2 Hz, 1H), 6.69 (d, J = 8.2 Hz, 1H), 6.08 (d, J = 2.5 Hz, 1H), 5.31 (t, J = 7.5 Hz, 1H), 3.89 (s, 2H), 3.71 (s, 1H), 3.53-3.41 (m, 0H), 2.65 (d, J = 9.8 Hz, 3H), 2.42 (s, 1H), 2.33 (s, 4H), 2.21-2.07 (m, 8H), 1.31 (d, J = 17.2 Hz, 2H), 0.94 (t, J = 7.3 Hz, 3H). MS (ES⁺) m/z 498.4 (M + H)⁺. |
| | N-(3-hydroxy-2,6-dimethylphenyl)-2- | |
| | ((1-(1-(4-methylpiperazin-1-yl)-1- | |
| | oxobutan-2-yl)-1H-pyrazol-3- | |
| | yl)amino)thiazole-5-carboxamide | |
| BAA- 534 | 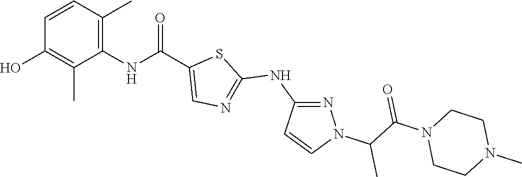 [SMILES: Cc1ccc(O)c(C)c1NC(=O)c1cnc(Nc2ccn(C(C)C(=O)N3CCN(C)CC3)n2)s1] | ¹H NMR (400 MHz, MeOD) δ 8.43 (s, 1H), 8.04 (s, 1H), 7.64 (d, J = 2.5 Hz, 1H), 6.92 (d, J = 8.3 Hz, 1H), 6.69 (d, J = 8.3 Hz, 1H), 6.07 (d, J = 2.5 Hz, 1H), 5.56 (q, J = 6.8 Hz, 1H), 3.87 (s, 2H), 3.70 (s, 1H), 3.59-3.43 (m, 1H), 2.66 (s, 3H), 2.51 (s, 1H), 2.37 (s, 3H), 2.17 (s, 3H), 2.11 (s, 3H), 1.66 (d, J = 6.8 Hz, 3H), 1.31 (d, J = 17.2 Hz, 2H). MS (ES⁺) m/z 484.3 (M + H)⁺. |
| | N-(3-hydroxy-2,6-dimethylphenyl)-2- | |
| | ((1-(1-(4-methylpiperazin-1-yl)-1- | |
| | oxopropan-2-yl)-1H-pyrazol-3- | |
| | yl)amino)thiazole-5-carboxamide | |
| BAA- 555 | 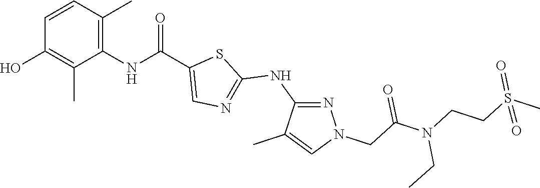 [SMILES: CCN(CCS(C)(=O)=O)C(=O)Cn1cc(C)c(Nc2ncc(C(=O)Nc3c(C)ccc(O)c3C)s2)n1] | ¹H NMR (400 MHz, DMSO-d₆) δ 10.64 (s, 1H), 9.42 (s, 1H), 9.12 (s, 1H), 8.06 (s, 1H), 7.42-7.33 (m, 1H), 6.83 (d, J = 8.2 Hz, 1H), 6.64 (d, J = 8.1 Hz, 1H), 5.07-4.84 (m, 2H), 3.77-3.56 (m, 2H), 3.56-3.37 (m, 2H), 3.04-2.94 (m, 3H), 2.02 (s, 3H), 1.97 (s, 3H), 1.94 (s, 3H), 1.16-0.93 (m, 3H). MS (ES⁺) m/z 535.2 (M + H)⁺. |
| | 2-((1-(2-(ethyl(2-(methylsulfonyl)ethyl) | |
| | amino)-2-oxoethyl)-4-methyl-1H- | |
| | pyrazol-3-yl)amino)-N-(3-hydroxy-2,6- | |
| | dimethylphenyl)thiazole-5- | |
| | carboxamide | |

### 2-((1-(2-(Ethyl(2-hydroxyethyl)amino)-2-oxoethyl)-4-methyl-1H-pyrazol-3-yl)amino)-N-(3-hydroxy-2,6-dimethylphenyl)thiazole-5-carboxamide( ) BAA-535

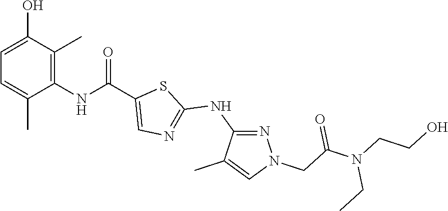 [SMILES: CCN(CCO)C(=O)Cn1cc(C)c(Nc2ncc(C(=O)Nc3c(C)ccc(O)c3C)s2)n1]

1-Propanephosphonic anhydride, 50 wt % in EtOAc (0.48 mL, 0.800 mmol) was added drop-wise to a stirred solution of N-(2-methoxyethyl)ethylamine (66 μL, 0.533 mmol), 2-[3-[5-[(3-methoxy-2,6-dimethyl-phenyl) carbamoyl]thiazol-2-yl]amino]-4-methyl-pyrazol-1-yl]acetic acid (221 mg, 0.533 mmol) and DIPEA (232 μL, 1.33 mmol) in THF (4 mL) at room temperature. The reaction mixture was stirred at 40° C. overnight, was then evaporated, and water (5 mL) added which precipitated a cream solid. The material was sonicated for 10 seconds to further aid precipitation. The solid was then collected by filtration under vacuum, the filter cake was washed with water (20 mL) and iso-hexane (20 mL), taken up in MeCN (5 mL) and evaporated to give 2-[[1-[2-[ethyl(2-methoxyethyl)amino]-2-oxo-ethyl]-4-methyl-pyrazol-3-yl]amino]-N-(3-methoxy-2,6-dimethyl-phenyl)thiazole-5-carboxamide (253 mg, 0.394 mmol, 73.94% yield) as an brown oil.MS (ES+) m/z=501.4 [M+H]+. The material from the first step was suspended in DCM (12 mL), boron tribromide 1M DCM (5.79 eq, 1 mL, 1 mmol) was added and the resulting mixture was stirred for 16 h at ambient temperature, under nitrogen. The reaction was then quenched with MeOH(10 mL), stirred for 10 minutes, evaporated, then water (5 ml) was added and the reaction mixture was evaporated again. The crude material was then purified by prep HPLC to afford the title compound
(6.4 mg, 0.0126 mmol, 2.90% yield) as a yellow solid. MS (ES+) m/z=473.2 [M+H]+. ¹H NMR
(400 MHz, DMSO-D6) o 10.65 (s, 1H), 9.45 (s, 1H), 9.15 (s, 1H), 8.09 (s, 1H), 7.45-7.35 (m, 1H), 6.87 (d, J=8.2 Hz, 1H), 6.67 (d, J=8.2 Hz, 1H), 5.02-4.92 (m, 2H), 5.12-4.60 (m, 1H), 3.59-3.52 (m, 1H), 3.51-3.38 (m, 3H), 3.32-3.25 (m, 2H), 2.06 (s, 3H), 2.00 (s, 3H), 1.98 (s, 3H), 1.17 -0.99 (m, 3H).

### N-(3-Hydroxy-2.6-dimethylphenyl)-2-((1-(2-((2-hydroxyethyl)(methyl)amino)-2-oxoethyl)-4-methyl-1H-pyrazol-3-yl)amino)thiazole-5-carboxamide( ) BAA-524

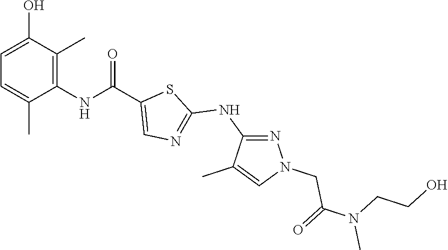 [SMILES: Cc1cn(CC(=O)N(C)CCO)nc1Nc1ncc(C(=O)Nc2c(C)ccc(O)c2C)s1]

1-Propanephosphonic anhydride, 50 wt % in EtOAc (0.22 mL, 0.370 mmol) was added drop-wise to a stirred solution of N-(2-Methoxyethyl)methylamine (27 μL, 0.247 mmol), 2-[3-[5-[(3-methoxy-2,6-dimethyl-phenyl) carbamoyl]thiazol-2-yl]amino]-4-methyl-pyrazol-1-yl]acetic acid (103 mg, 0.247 mmol) and DIPEA (107 μL, 0.617 mmol) in THF (2 mL). The reaction mixture was stirred at 40° C. for 16 h, then evaporated, water (5 mL) was then added which precipitated a cream solid, sonicated for 10 seconds to further aid precipitation. The solid was collected by filtration under vacuum, the filter cake was then washed with water (20 mL), then iso-hexane (20 mL), taken up in MeCN (5 mL) and evaporated to give N-(3-methoxy-2,6-dimethyl-phenyl)-2-[1-[2-[2-methoxyethyl(methyl)amino]-2-oxo-ethyl]-4-methyl-pyrazol-3-yl]amino]thiazole-5-carboxamide
(102 mg, 0.115 mmol, 46.71% yield).MS (ES+) m/z=487.1 [M+H]+. The material from the first step was suspended in DCM (29 mL), boron tribromide 1M DCM (5.79 eq, 2.5 mL, 2.51 mmol) was added and the resulting mixture was stirred for 2 h at ambient temperature, under nitrogen. The reaction was then quenched with MeOH(10 mL), stirred for 10 minutes, evaporated, then water (5 ml) was added and the reaction mixture was evaporated again. The crude material was then purified by prep HPLC to afford the title compound (5.2 mg, 0.0107 mmol, 5.98% yield) as a white solid. MS (ES+) m/z=459.2 [M+H]+. ¹H NMR (400 MHz, DMSO-D6) δ 10.65 (s, 1H), 9.45 (s, 1H), 9.16 (s, 1H), 8.09 (d, J=1.2 Hz, 1H), 7.40-7.34 (m, 1H), 6.87 (d, J=8.2 Hz, 1H), 6.67 (d, J=8.2 Hz, 1H), 5.06-4.93 (m, 2H), 4.99-4.62 (m, 1H), 3.65-3.55 (m, 1H), 3.54-3.35 (m, 3H), 3.11-2.78 (m, 3H), 2.06 (s, 3H), 2.02-1.99 (m, 3H), 1.98 (s, 3H).

### N-(4-bromo-3-hydroxy-2,6-dimethylphenyl)-2-((1-(2-(diethylamino)-2-oxoethyl)-4-methyl-1H-pyrazol-3-yl)amino)thiazole-5-carboxamide( ) BAA-608

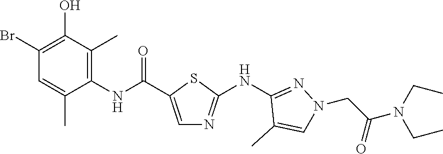 [SMILES: CCN(CC)C(=O)Cn1cc(C)c(Nc2ncc(C(=O)Nc3c(C)cc(Br)c(O)c3C)s2)n1]

Triethylamine (3.75 eq, 0.30 mL, 2.15 mmol), Diethylamine (3.37 eq, 0.20 mL, 1.93 mmol), and HATU (1.15 eq, 250 mg, 0.657 mmol) were added to a solution of 2-[3-[5-[(3-methoxy-2,6-dimethyl-phenyl) carbamoyl]thiazol-2-yl]amino]-4-methyl-pyrazol-1-yl]acetic acid (1.00 eq, 265 mg, 0.574 mmol) in DMF (5 mL). The reaction mixture was stirred at ambient for 18 h, then diluted with ethyl acetate and washed with water and brine. The organic phase were dried over sodium sulfate, filtered and evaporated. The crude material was purified by column chromatography over silica (24 g cartridge) eluting with a gradient of MeOH(0% to 20%; v/v) in DCM [note 3] to afford t2-[1-[2-(diethylamino)-2-oxo-ethyl]-4-methyl-pyrazol-3-yl]amino]-N-(3-methoxy-2,6-dimethyl-phenyl)thiazole-5-carboxamide (350 mg, 0.379 mmol, 66.08% yield) as a brown gum. MS (ES+) m/z=471.2 [M+H]+.

The product from the first step (1.00 eq, 350 mg, 0.379 mmol) was dissolved in DCM (10 mL), and boron tribromide [1M DCM](6.59 eq, 2.5 mL, 2.50 mmol) was added over 2 min. The reaction mixture was stirred overnight. MeOH(10 mL) was added, and the mixture was stirred at rt for 1 hour and evaporated. The crude material was purified by column chromatography over C18 (45 g cartridge) eluting with a gradient of MeCN (0.1% NH₃)(5% to 95%; v/v) in water (0.1% NH₃)N-(4- to afford the title compound (41 mg, 0.0720 mmol, 18.98% yield) as a white solid.MS (ES+) m/z=535.0/537.0 mono Br [M+H]+. ¹H NMR (400 MHz, DMSO-D6) δ 10.66 (s, 1H), 9.50 (s, 1H), 8.84 (s, 1H), 8.06 (s, 1H), 7.38 (d, J=0.9 Hz, 1H), 7.23 (s, 1H), 4.88 (s, 2H), 3.35 (q, J=7.1 Hz, 2H), 3.22 (q, J=7.1 Hz, 2H), 2.03 (s, 6H), 1.96 (d, J=0.8 Hz, 3H), 1.10 (t, J=7.1 Hz, 3H), 0.98 (t, J=7.1 Hz, 3H).

General Method U2. Synthesis of phenol-amides from 3-methoxy-2-[3-[[5-[(3-methoxy-2,6-dimethyl-phenyl) carbamoyl]thiazol-2-yl]amino]-4-methyl-pyrazol-1-yl]propanoic acid coupling with amines and concomitant aromatic and aliphatic methoxy deprotection

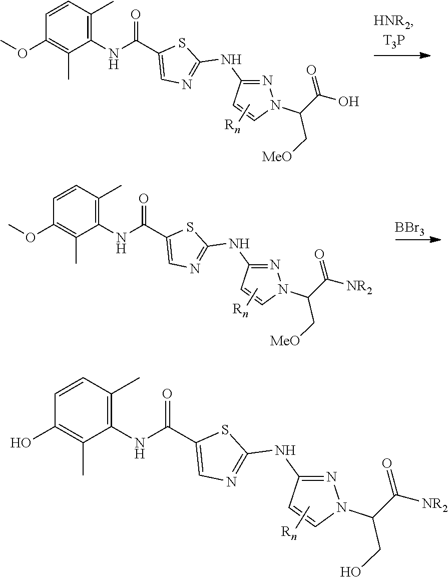

3-Methoxy-2-[3-[5-[(3-methoxy-2,6-dimethyl-phenyl) carbamoyl]thiazol-2-yl]amino]-4-methyl-pyrazol-1-yl]propanoic acid or its caesium salt (1 eq) was suspended in EtOAc or THF, amine (1-5 eq), DIPEA (3-10 eq) and 1-propanephosphonic anhydride, 50 wt % in EtOAc (1.5-4 eq) were added, the reaction mixture was heated to 65° C. for 18 h then was diluted with EtOAc (50 mL) and H₂O (50 mL). The organic layer was washed with water (2×50 mL) and brine (50 mL), before being dried over Na₂SO₄, filtered and the filtrates concentrated under reduced pressure to afford the methoxy-protected amide intermediate. The product from the first step (1 eq) was dissolved in DCM, boron tribromide, 1 M in DCM (6 eq) was added and the reaction mixture was stirred at r.t. for 3-5 h. The reaction mixture was diluted with MeOH(10 mL), concentrated under reduced pressure and the crude purified by prep HPLC to afford the product.

### 2-((1-(3-Hydroxy-1-oxo-1-(pyrrolidin-1-yl)propan-2-yl)-4-methyl-1H-pyrazol-3-yl)amino)-N-(3-hydroxy-2,6-dimethylphenyl)thiazole-5-carboxamide( ) BAA-583

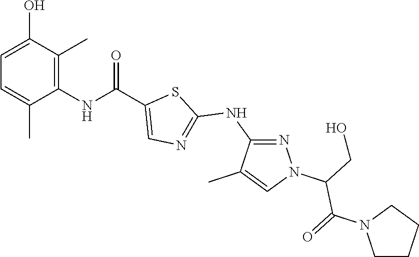 [SMILES: Cc1cn(C(CO)C(=O)N2CCCC2)nc1Nc1ncc(C(=O)Nc2c(C)ccc(O)c2C)s1]

Prepared as described in method U2 from caesium 3-methoxy-2-[3-[5-[(3-methoxy-2,6-dimethyl-phenyl) carbamoyl]thiazol-2-yl]amino]-4-methyl-pyrazol-1-yl]propanoate (1.00 eq, 225 mg, 0.247 mmol), pyrrolidine (5 eq, 101 μL, 1.24 mmol), DIPEA (10.0 eq, 431 μL, 2.47 mmol) and 1-propanephosphonic anhydride, 50 wt % in EtOAc (4.00 eq, 291 μL, 0.989 mmol) in EtOAc (5.0 mL) followed by treatment with boron tribromide, 1 M in DCM (6.01 eq, 750 μL, 0.750 mmol) to afford the title compound (18 mg, 0.0361 mmol, 28.89% yield) as a pale-yellow solid after prep HPLC purification. MS (ES+) m/z=485.4, [M+H]⁺; ¹H NMR (400 MHz, DMSO-d₆) δ 10.54 (br s, 1H), 9.27 (s, 1H), 9.00 (br s, 1H), 7.92 (s, 1H), 7.35 (s, 1H), 6.71 (d, J=8.2 Hz, 1H), 6.51 (d, J=8.2 Hz, 1H), 4.94-4.87 (m, 1H), 3.81-3.72 (m, 1H), 3.72-3.63 (m, 1H), 3.43 (dt, J=10.0, 6.7 Hz, 1H), 3.26 (dt, J=10.0, 6.9 Hz, 1H), 3.15-3.11 (m, 2H), 1.89 (s, 3H), 1.82 (s, 3H), 1.81 (s, 3H), 1.75-1.64 (m, 2H), 1.64-1.50 (m, 2H).

The following example compounds were prepared similarly using Method U2 with appropriately substituted amines.

| # | Structure & Name | Analytical Data |
|---|---|---|
| BAA-576 | 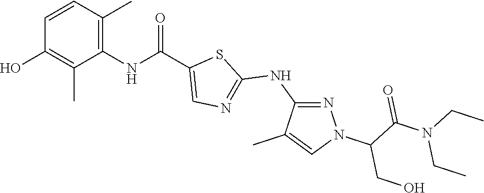 [SMILES: CCN(CC)C(=O)C(CO)n1cc(C)c(Nc2ncc(C(=O)Nc3c(C)ccc(O)c3C)s2)n1] | ¹H NMR (400 MHz, DMSO-d₆) δ 10.73 (s, 1H), 9.46 (s, 1H), 9.17 (s, 1H), 8.10 (s, 1H), 7.52 (s, 1H), 6.87 (d, J = 8.2 Hz, 1H), 6.68 (d, J = 8.2 Hz, 1H), 5.18 (t, J = 6.9 Hz, 1H), 5.07 (t, J = 5.8 Hz, 1H), 3.96- 3.75 (m, 2H), 3.53-3.39 (m, J = 7.3 Hz, 2H), 3.32-3.20 (m, 2H), 2.06 (s, 3H), 2.00 (s, 3H), 1.98 (s, 3H), 1.11 (t, J = 7.1 Hz, 3H), 1.00 (t, J = 7.1 Hz, 3H). MS (ES⁺) m/z 487.2 (M + H)⁺. |
| | 2-((1-(1-(diethylamino)-3-hydroxy-1- | |
| | oxopropan-2-yl)-4-methyl-1H-pyrazol- | |
| | 3-yl)amino)-N-(3-hydroxy-2,6- | |
| | dimethylphenyl)thiazole-5- | |
| | carboxamide | |
| BAA-578 | 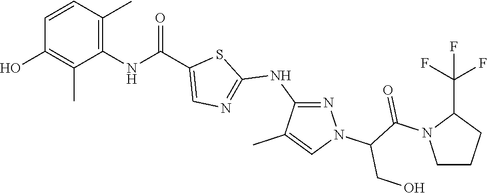 [SMILES: Cc1cn(C(CO)C(=O)N2CCCC2C(F)(F)F)nc1Nc1ncc(C(=O)Nc2c(C)ccc(O)c2C)s1] | ¹H NMR (400 MHz, MeOD) δ 8.01 (d, J = 1.1 Hz, 1H), 7.51 (d, J = 1.1 Hz, 1H), 6.89 (d, J = 8.2 Hz, 1H), 6.66 (d, J = 8.2 Hz, 1H), 5.25 (t, J = 6.5 Hz, 1H), 4.85-4.73 (m, 1H), 4.10-4.04 (m, 2H), 3.86 (d, J = 8.8 Hz, 1H), 3.77 (s, 1H), 2.17- 1.99 (m, 13H). MS (ES⁺) m/z 553.3 (M + H)⁺. |
| | 2-((1-(3-hydroxy-1-oxo-1-(2- | |
| | (trifluoromethyl)pyrrolidin-1-yl)propan- | |
| | 2-yl)-4-methyl-1H-pyrazol-3-yl)amino)- | |
| | N-(3-hydroxy-2,6-dimethylphenyl) | |
| | thiazole-5-carboxamide | |
| BAA-591 | 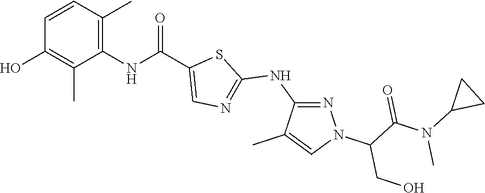 [SMILES: Cc1cn(C(CO)C(=O)N(C)C2CC2)nc1Nc1ncc(C(=O)Nc2c(C)ccc(O)c2C)s1] | ¹H NMR (400 MHz, DMSO-d₆) δ 8.06 (s, 1H), 7.49 (s, 1H), 6.83 (s, J = 8.2 Hz, 1H), 6.63 (d, J = 8.2 Hz, 1H), 5.65 (t, J = 7.0 Hz, 1H), 5.11 (br t, J = 5.2 Hz, 1H), 3.94-3.87 (m, 1H), 3.83-3.75 (m, 1H), 2.78 (s, 3H), 2.68 (p, J = 5.8 Hz, 1H), 2.02 (s, 3H), 1.95 (s, 3H), 1.94 (s, 3H), 0.86 (tt, J = 13.5, 5.7 Hz, 4H). MS (ES⁺) m/z 485.4 (M + H)⁺. |
| | 2-((1-(1-(cyclopropyl(methyl)amino)-3- | |
| | hydroxy-1-oxopropan-2-yl)-4-methyl- | |
| | 1H-pyrazol-3-yl)amino)-N-(3-hydroxy- | |
| | 2,6-dimethylphenyl)thiazole-5- | |
| | carboxamide | |
| BAA-592 | 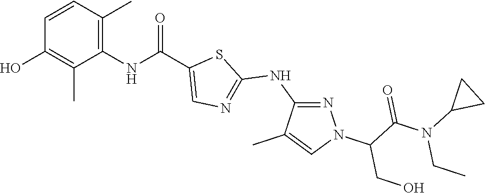 [SMILES: CCN(C(=O)C(CO)n1cc(C)c(Nc2ncc(C(=O)Nc3c(C)ccc(O)c3C)s2)n1)C1CC1] | ¹H NMR (400 MHz, DMSO-d₆) δ 9.11 (s, 1H), 7.98 (br s, 1H), 7.48 (d, J = 1.0 Hz, 1H), 6.87 (d, J = 8.2 Hz, 1H), 6.69 (d, J = 8.2 Hz, 1H), 5.64 (s, 1H), 3.98 (dd, J = 11.0, 6.5 Hz, 1H), 3.89 (dd, J = 10.9, 7.0 Hz, 1H), 3.37 (qd, J = 7.1, 2.2 Hz, 2H), 2.75 (tt, J = 7.0, 4.0 Hz, 1H), 2.09 (d, J = 2.5 Hz, 3H), 2.03 (d, J = 2.3 Hz, 3H), 2.01 (d, J = 0.8 Hz, 3H), 1.05 (t, J = 7.1 Hz, 3H), 0.93 (br d, J = 7.0 Hz, 2H), 0.85 (br s, 2H); MS (ES⁺) m/z 499.4 (M + H)⁺. |
| | 2-((1-(1-(cyclopropyl(ethyl)amino)-3- | |
| | hydroxy-1-oxopropan-2-yl)-4-methyl- | |
| | 1H-pyrazol-3-yl)amino)-N-(3-hydroxy- | |
| | 2,6-dimethylphenyl)thiazole-5- | |
| | carboxamide | |

### 2-(3-((5-((3-Hydroxy-2,6-dimethylphenyl)carbamoyl)thiazol-2-yl)amino)-4-methyl-1H-pyrazol-1-yl)-3-methoxypropanoic acid

### Route to Amides Via Silyl Protected Phenols

### 2-(3-((5-((3-((tert-Butyldimethylsilyl)oxy)-2,6-dimethylphenyl)carbamoyl)thiazol-2-yl)amino)-4-methyl-1H-pyrazol-1-yl) acetic acid

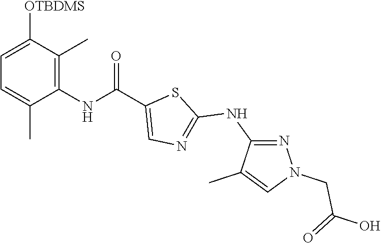 [SMILES: Cc1cn(CC(=O)O)nc1Nc1ncc(C(=O)Nc2c(C)ccc(O[Si](C)(C)C(C)(C)C)c2C)s1]

A solution of 2-bromo-N-[3-[tert-butyl(dimethyl) silyl]oxy-2,6-dimethyl-phenyl]thiazole-5-carboxamide (2.00 g, 4.53 mmol) and ethyl 2-(3-amino-4-methyl-pyrazol-1-yl)acetate (1.74 g, 9.51 mmol) in 1,4-dioxane (53.3 mL) was sparged with nitrogen for 5 minutes. To the vessel was added potassium carbonate 325 mesh (1.69 g, 12.2 mmol), 4,5-bis(diphenylphosphino)-9,9-dimethylxanthene (Xantphos)(524 mg, 0.906 mmol) and palladium (II)acetate (102 mg, 0.453 mmol) and the mixture degassed with nitrogen for 5 more minutes. The reaction then stirred at 80° C. for 16 h. The reaction mixture was then evaporated, taken up in EtOAc (20 mL) and filtered through a pad of celite, eluting with EtOAc (200 mL). The filtrate was then evaporated and the crude material was purified by column chromatography over silica (80 g cartridge) eluting with a gradient of EtOAc (30% to 100%; v/v) in iso-hexane to give RH-0524-031-S3 as a beige solid ethyl 2-[3-[5-[3-[tert-butyl(dimethyl) silyl]oxy-2,6-dimethyl-phenyl]carbamoyl]thiazol-2-yl]amino]-4-methyl-pyrazol-1-yl]acetate (1527 mg, 2.50 mmol, 55.17% yield) MS (ES+) m/z=544.2 [M+H]⁺. 1H NMR (400 MHz, CHLOROFORM-D) δ 8.17 (s, 1H), 7.88 (s, 1H), 7.21 (s, 1H), 7.00 (s, 1H), 6.94 (d, J=8.2 Hz, 1H), 6.69 (d, J=8.3 Hz, 1H), 4.80 (s, 2H), 4.24 (q, J=7.1 Hz, 2H), 2.21 (s, 3H), 2.14 (s, 3H), 2.04 (s, 3H), 1.30 (t, J=7.1 Hz, 3H), 1.00 (s, 9H), 0.20 (s, 6H).

The product from the first step (995 mg, 1.83 mmol) was dissolved in THF (18.3 mL), potassium trimethylsilanolate (915 μL, 1.83 mmol) solution in THF 2M was added and the resulting mixture was stirred at 50° C. for 1 h. The reaction mixture was recharged with potassium trimethylsilanolate (0.37 mL, 0.732 mmol) at 50° C. and left to stir for 2h. Water (100 mL) was added to the reaction mixture, then extracted with DCM (2×100 mL). The combined organic extracts were evaporated, the crude material was then dissolved in THF (18.3 mL), then more potassium trimethylsilanolate (915 μL, 1.83 mmol) was added, the reaction mixture was heated to 50° C. and left to stir for 3h, and for further 16 h at ambient temperature. The reaction mixture was then diluted with brine (100 mL), then extracted with DCM (2×100 mL), the combined organic extracts were then evaporated to give O2-[3-[5-[3-[tert-butyl(dimethyl) silyl]oxy-2,6-dimethyl-phenyl]carbamoyl]thiazol-2-yl]amino]-4-methyl-pyrazol-1-yl]acetic acid (841 mg, 1.29 mmol, 70.41% yield) as an orange solid. MS (ES+) m/z=516.2 [M+H]+. ¹H NMR (400 MHz, DMSO-D6) δ 10.54 (s, 1H), 9.52 (s, 1H), 8.09 (s, 1H), 7.32 (s, 1H), 6.96 (d, J=8.4 Hz, 1H), 6.69 (d, J=8.3 Hz, 1H), 4.28 (s, 2H), 2.10 (s, 3H), 2.02 (s, 3H), 1.98 (s, 3H), 0.98 (s, 9H), 0.19 (s, 6H). 1H exchangeable not observed.

### Methyl 2-(3-((5-((3-((tert-butyldimethylsilyl)oxy)-2.6-dimethylphenyl)carbamoyl)thiazol-2-yl)amino)-4-methyl-1H-pyrazol-1-yl)-3-methoxypropanoate

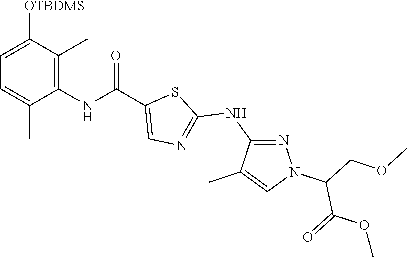 [SMILES: COCC(C(=O)OC)n1cc(C)c(Nc2ncc(C(=O)Nc3c(C)ccc(O[Si](C)(C)C(C)(C)C)c3C)s2)n1]

A solution of 2-bromo-N-[3-[tert-butyl(dimethyl) silyl]oxy-2,6-dimethyl-phenyl]thiazole-5-carboxamide (271 mg, 0.614 mmol) and methyl 2-(3-amino-4-methyl-pyrazol-1-yl)-3-methoxy-propanoate (262 mg, 1.23 mmol) in 1,4-dioxane (7 mL) was sparged with nitrogen for 5 minutes, potassium carbonate 325 mesh (229 mg, 1.66 mmol), Xantphos (71 mg, 0.123 mmol) and palladium (II)acetate (14 mg, 0.0614 mmol) were added and the mixture degassed with nitrogen for 5 more minutes. The reaction was stirred at 80° C. for 16 h, filtered though a plug of celite and evaporated. Th crude was purified by column chromatography over silica (24 g cartridge) eluting with a gradient of EtOAc (20% to 100%; v/v) in iso-hexane to afford the title product (86 mg, 0.109 mmol, 17.82% yield). MS (ES+) m/z=574.2 [M+H]+.

General Method V. Synthesis of Phenol-Amides from Unsubstituted or Substituted 2-(3-((5-((3-((Tert-Butyldimethylsilyl)Oxy)-2,6-Dimethylphenyl)Carbamoyl)Thiazol-2-Yl)Amino)-1H-Pyrazol-1-Yl) Acetic Acid Coupling with Amines Using T3P and Silyl Deprotection with HCl

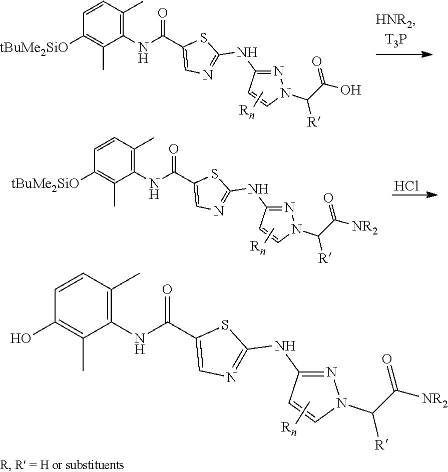

To a solution of the carboxylic acid (1 eq), amine (1 eq) and DIPEA (3 eq) in THF was added 1-propanephosphonic anhydride (1.5 eq). The resulting mixture was heated to 40° C. and left to stir for 16 h, then it was taken-up in methanol (5 mL), and evaporated to give the silylated amide, which was used in the next step without further purification. The intermediate was dissolved in MeOH, and HCl 4M in dioxane was added. The reaction was stirred for 16 h at rt, evaporated and purified by preparative HPLC to provide the desired product.

### N-(3-Hydroxy-2,6-dimethylphenyl)-2-((1-(2-((2-methoxyethyl)(methyl)amino)-2-oxoethyl)-4-methyl-1H-pyrazol-3-yl)amino)thiazole-5-carboxamide( ) BAA-549

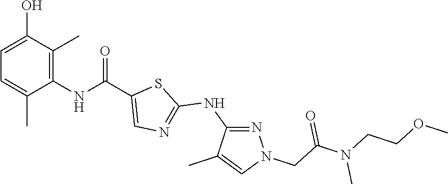 [SMILES: COCCN(C)C(=O)Cn1cc(C)c(Nc2ncc(C(=O)Nc3c(C)ccc(O)c3C)s2)n1]

Prepared as described in method V from 1-propanephosphonic anhydride, 50% wt solution in EtOAc (0.22 mL, 0.372 mmol), 2-[3-[5-[3-[tert-butyl(dimethyl) silyl]oxy-2,6-dimethyl-phenyl]carbamoyl]thiazol-2-yl]amino]-4-methyl-pyrazol-1-yl]acetic acid (160 mg, 0.248 mmol), N-(2-Methoxyethyl)methylamine (27 μL, 0.248 mmol) and DIPEA (0.11 mL, 0.621 mmol) in THF (2.07 mL), followed by deprotection with HCl 4M in dioxane (1 mL, 4 mmol) and purification by preparative HPLC to afford the title compound (2.7 mg, 0.00560 mmol, 3.89% yield). MS (ES+) m/z=473.3 [M+H]+. ¹H NMR (400 MHz, DMSO-D6) δ 10.65 (s, 1H), 9.45 (s, 1H), 9.21 (s, 1H), 8.09 (s, 1H), 7.38 (d, J=1.0 Hz, 1H), 6.87 (d, J=8.2 Hz, 1H), 6.67 (d, J=8.2 Hz, 1H), 4.98 (d, J=3.9 Hz, 2H), 3.56 (t, J=5.0 Hz, 1H), 3.51 (t, J=4.8 Hz, 1H), 3.48-3.41 (m, 2H), 3.31-3.22 (m, 3H), 3.09 -2.82 (m, 3H), 2.06 (s, 3H), 2.00 (s, 3H), 1.98 (s, 3H).

The following example compounds were prepared similarly using Method V with appropriately substituted, silyl-protected, carboxylic acids and amines.

| # | Structure & Name | Analytical Data |
|---|---|---|
| BAA-538 | 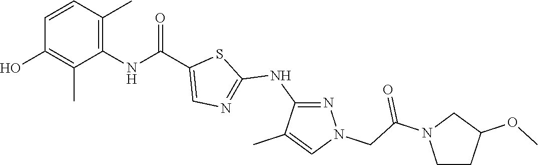 [SMILES: COC1CCN(C(=O)Cn2cc(C)c(Nc3ncc(C(=O)Nc4c(C)ccc(O)c4C)s3)n2)C1] | ¹H NMR (400 MHz, DMSO-d₆) δ 10.64 (s, 1H), 9.46 (s, 1H), 9.20 (s, 1H), 8.09 (s, 1H), 7.41 (s, 1H), 6.87 (d, J = 8.2 Hz, 1H), 6.67 (d, J = 8.1 Hz, 1H), 4.95-4.80 (m, 2H), 4.06-3.87 (m, 1H), 3.73-3.36 (m, 4H), 3.25-3.19 (m, 3H), 2.08-1.95 (m, 10H), 1.93-1.82 (m, 1H). MS (ES⁺) m/z 485.2 (M + H)⁺. |
| | N-(3-hydroxy-2,6-dimethylphenyl)-2- | |
| | ((1-(2-(3-methoxypyrrolidin-1-yl)-2- | |
| | oxoethyl)-4-methyl-1H-pyrazol-3- | |
| | yl)amino)thiazole-5-carboxamide | |
| BAA-542 | 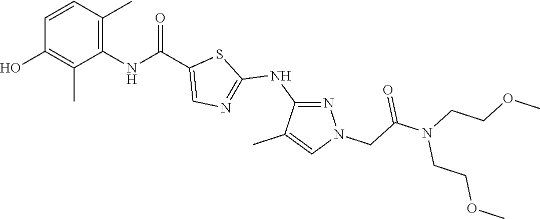 [SMILES: COCCN(CCOC)C(=O)Cn1cc(C)c(Nc2ncc(C(=O)Nc3c(C)ccc(O)c3C)s2)n1] | ¹H NMR (400 MHz, DMSO-d₆) δ 10.66 (s, 1H), 9.45 (s, 1H), 9.16 (s, 1H), 8.09 (s, 1H), 7.39 (s, 1H), 6.87 (d, J = 8.2 Hz, 1H), 6.67 (d, J = 8.2 Hz, 1H), 5.01 (s, 2H), 3.30 (s, 3H), 3.21 (s, 3H), 2.06 (s, 3H), 2.00 (s, 3H), 1.97 (s, 3H). 8H obscured under water signal. MS (ES⁺) m/z 517.2 (M + H)⁺. |
| | 2-((1-(2-(bis(2-methoxyethyl)amino)-2- | |
| | oxoethyl)-4-methyl-1H-pyrazol-3- | |
| | yl)amino)-N-(3-hydroxy-2,6-dimethyl- | |
| | phenyl)thiazole-5-carboxamide | |
| BAA-550 | 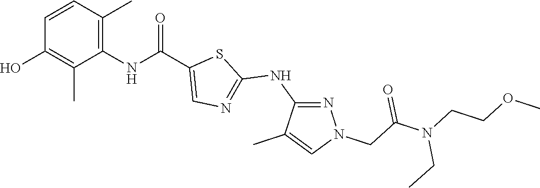 [SMILES: CCN(CCOC)C(=O)Cn1cc(C)c(Nc2ncc(C(=O)Nc3c(C)ccc(O)c3C)s2)n1] | ¹H NMR (400 MHz, DMSO-d₆) δ 9.46 (s, 1H), 9.25 (s, 1H), 8.10 (s, 1H), 7.45- 7.34 (m, 1H), 6.87 (d, J = 8.2 Hz, 1H), 6.68 (d, J = 8.2 Hz, 1H), 5.04-4.94 (m, 2H), 3.61-3.17 (m, 13H), 2.06 (s, 3H), 2.00 (s, 3H), 1.98 (s, 3H), 1.20-0.96 (m, 3H). MS (ES⁺) m/z 487.3 (M + H)⁺. |
| | 2-((1-(2-(ethyl(2-methoxyethyl)amino)- | |
| | 2-oxoethyl)-4-methyl-1H-pyrazol-3- | |
| | yl)amino)-N-(3-hydroxy-2,6- | |
| | dimethylphenyl)thiazole-5- | |
| | carboxamide | |
| BAA-551 | 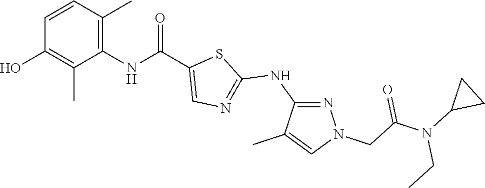 [SMILES: CCN(C(=O)Cn1cc(C)c(Nc2ncc(C(=O)Nc3c(C)ccc(O)c3C)s2)n1)C1CC1] | ¹H NMR (400 MHz, DMSO-d₆) δ 10.45 (s, 1H), 9.34 (s, 1H), 9.00 (s, 1H), 8.07 (s, 1H), 7.41 (d, J = 0.9 Hz, 1H), 6.87 (d, J = 8.2 Hz, 1H), 6.68 (d, J = 8.2 Hz, 1H), 5.05 (s, 2H), 3.33 (q, J = 7.1 Hz, 2H), 2.82 (s, 1H), 2.07 (s, 3H), 2.01 (s, 3H), 2.00 (s, 3H), 1.06 (t, J = 7.1 Hz, 3H), 0.88 (d, J = 15.9 Hz, 4H). MS (ES⁺) m/z 469.3 (M + H)⁺. |
| | 2-((1-(2-(cyclopropyl(ethyl)amino)-2- | |
| | oxoethyl)-4-methyl-1H-pyrazol-3- | |
| | yl)amino)-N-(3-hydroxy-2,6- | |
| | dimethylphenyl)thiazole-5- | |
| | carboxamide | |
| BAA-554 | 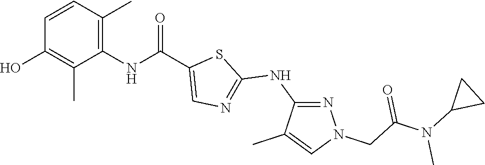 [SMILES: Cc1cn(CC(=O)N(C)C2CC2)nc1Nc1ncc(C(=O)Nc2c(C)ccc(O)c2C)s1] | ¹H NMR (400 MHz, DMSO-d₆) δ 10.57 (s, 1H), 9.45 (s, 1H), 9.17 (s, 1H), 8.09 (s, 1H), 7.41 (s, 1H), 6.87 (d, J = 8.2 Hz, 1H), 6.67 (d, J = 8.2 Hz, 1H), 5.08 (s, 2H), 2.82 (s, 4H), 2.06 (s, 3H), 2.01 (s, 3H), 1.98 (s, 3H), 0.91-0.81 (m, 4H). MS (ES⁺) m/z 455.3 (M + H)⁺. |
| | 2-((1-(2-(cyclopropyl(methyl)amino)-2- | |
| | oxoethyl)-4-methyl-1H-pyrazol-3- | |
| | yl)amino)-N-(3-hydroxy-2,6- | |
| | dimethylphenyl)thiazole-5- | |
| | carboxamide | |

General Method W. Synthesis of Phenol-Amides from Unsubstituted or Substituted 2-(3-((5-((3-((Tert-Butyldimethylsilyl)Oxy)-2,6-Dimethylphenyl)Carbamoyl)Thiazol-2-Yl)Amino)-1H-Pyrazol-1-Yl) Acetic Acid Coupling with Amines Using HATU and Silyl Deprotection with Tetraethylammonium fluoride

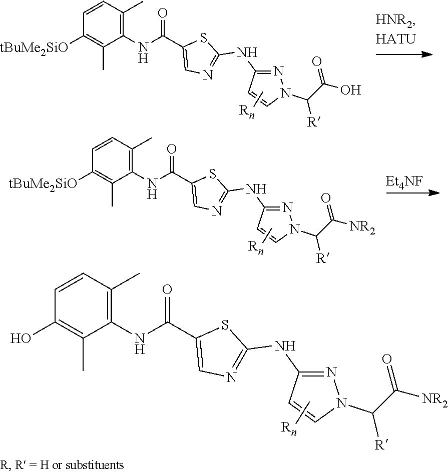

To a solution of the carboxylic acid (1 eq), amine (2 eq) and DIPEA (6-7 eq) in DMF was added HATU (1.2-1.5 eq). The resulting mixture was stirred at room temperature for 3 h, diluted with AcOEt, washed with NaHCO3aq, dried, filtered and evaporated to afford the silylated amide. The intermediate was dissolved in MeOH, and tetraethylammonium fluoride hydrate (7 eq) was added. The reaction was stirred for 3 h at rt, evaporated and purified by preparative HPLC to provide the desired product.

### N-(3-Hydroxy-2,6-dimethyl-phenyl)-2-[[1-[2-[(2R)-2-(methoxymethyl)pyrrolidin-1-yl]-2-oxo-ethyl]-4-methyl-pyrazol-3-yl]amino]thiazole-5-carboxamide( ) BAA-599

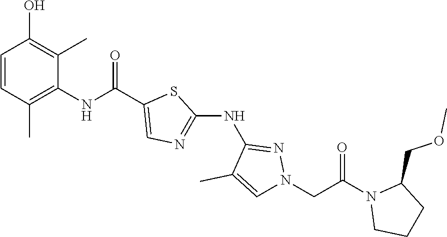 [SMILES: COC[C@H]1CCCN1C(=O)Cn1cc(C)c(Nc2ncc(C(=O)Nc3c(C)ccc(O)c3C)s2)n1]

Prepared as described in method W from R)-(+)-2-(methoxymethyl) pyrrolidine (1.96 eq, 35 mg, 0.304 mmol), 2-[3-[[5-[3-[tert-butyl(dimethyl) silyl]oxy-2,6-dimethyl-phenyl]carbamoyl]thiazol-2-yl]amino]-4-methyl-pyrazol-1-yl]acetic acid (1.00 eq, 80 mg, 0.155 mmol), HATU (1.19 eq, 70 mg, 0.184 mmol) and DIPEA (7.40 eq, 0.20 mL, 1.15 mmol) in DMF (5 mL). In the second step, the intermediate (1.00 eq, 100 mg, 0.147 mmol) in methanol (3 mL) and THF (3 mL) was deprotected with tetraethylammonium fluoride hydrate (6.51 eq, 160 mg, 0.957 mmol) and purified by preparative HPLC to afford the title compound (39 mg, 0.0782 mmol, 53.26% yield) as an off-white solid. MS (ES+) m/z=, m/z=499.4 [M+H]+. ¹H NMR (400 MHz, DMSO-D6) δ 10.65 (s, 1H), 9.42 (s, 1H), 9.12 (s, 1H), 8.05 (s, 1H), 7.36 (d, J=4.1 Hz, 1H), 6.83 (d, J=8.2 Hz, 1H), 6.63 (d, J=8.2 Hz, 1H), 5.00-4.74 (m, 2H), 4.33-3.93 (m, 1H), 3.55-3.42 (m, 1H), 3.35 (td, J=10.1, 3.5 Hz, 1H), 3.28-3.15 (m, 5H), 2.02 (s, 3H), 1.98-1.95 (m, 3H), 1.93 (s, 3H), 1.84 (dd, J=48.7, 9.5 Hz, 4H)

The following example compounds were prepared similarly using Method W with appropriately substituted, silyl-protected, carboxylic acids and amines.

| # | Structure & Name | Analytical Data |
|---|---|---|
| BAA-598 | 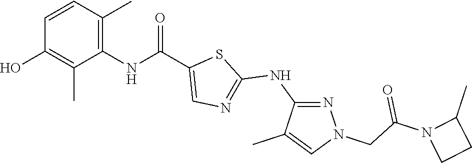 [SMILES: Cc1cn(CC(=O)N2CCC2C)nc1Nc1ncc(C(=O)Nc2c(C)ccc(O)c2C)s1] | ¹H NMR (400 MHz, DMSO-d₆) δ 10.67 (s, 1H), 9.42 (s, 1H), 9.12 (s, 1H), 8.06 (d, J = 1.3 Hz, 1H), 7.38 (dd, J = 2.0, 0.9 Hz, 1H), 6.83 (d, J = 8.2 Hz, 1H), 6.63 (d, J = 8.2 Hz, 1H), 4.78-4.51 (m, 2H), 4.42-3.97 (m, 2H), 3.74 (t, J = 7.8 Hz, 1H), 2.44-2.28 (m, 1H), 2.02 (s, 3H), 1.95 (d, J = 0.8 Hz, 3H), 1.94 (s, 3H), 1.80-1.68 (m, 1H), 1.36 (dd, J = 55.1, 6.3 Hz, 3H); MS (ES⁺) m/z 455.3 (M + H)⁺. |
| | N-(3-hydroxy-2,6-dimethylphenyl)-2- | |
| | ((4-methyl-1-(2-(2-methylazetidin-1- | |
| | yl)-2-oxoethyl)-1H-pyrazol-3- | |
| | yl)amino)thiazole-5-carboxamide | |
| BAA-600 | 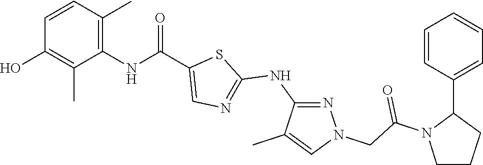 [SMILES: Cc1cn(CC(=O)N2CCCC2c2ccccc2)nc1Nc1ncc(C(=O)Nc2c(C)ccc(O)c2C)s1] | ¹H NMR (400 MHz, MeOD) δ 8.00 (dd, J = 3.4, 0.6 Hz, 1H), 7.43-7.07 (m, 6H), 6.93-6.85 (m, 1H), 6.66 (dd, J = 8.2, 3.3 Hz, 1H), 5.41-5.11 (m, 1H), 4.97 (q, J = 4.9 Hz, 1H), 4.54 (dd, J = 204.7, 15.9 Hz, 1H), 3.99-3.60 (m, 2H), 2.57-2.26 (m, 1H), 2.14 (d, J = 7.4 Hz, 3H), 2.09 (d, J = 7.4 Hz, 3H), 2.05-1.97 (m, 3H), 1.97-1.76 (m, 3H); MS (ES⁺) m/z 531.3 (M + H)⁺. |
| | N-(3-hydroxy-2,6-dimethylphenyl)-2- | |
| | ((4-methyl-1-(2-oxo-2-(2-phenyl | |
| | pyrrolidin-1-yl)ethyl)-1H-pyrazol-3- | |
| | yl)amino)thiazole-5-carboxamide | |
| BAA-605 | 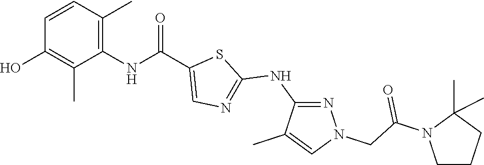 [SMILES: Cc1cn(CC(=O)N2CCCC2(C)C)nc1Nc1ncc(C(=O)Nc2c(C)ccc(O)c2C)s1] | ¹H NMR (400 MHz, DMSO-d₆) δ 10.60 (s, 1H), 9.40 (s, 1H), 9.09 (s, 1H), 8.05 (s, 1H), 7.34 (d, J = 0.9 Hz, 1H), 6.83 (d, J = 8.2 Hz, 1H), 6.63 (d, J = 8.2 Hz, 1H), 4.75 (s, 2H), 3.53 (t, J = 6.7 Hz, 2H), 2.02 (s, 3H), 1.99-1.95 (m, 3H), 1.94 (s, 3H), 1.79 (p, J = 6.9 Hz, 2H), 1.68 (t, J = 6.6 Hz, 2H), 1.31 (s, 6H). MS (ES⁺) m/z 483.2 (M + H)⁺. |
| | 2-((1-(2-(2,2-dimethylpyrrolidin-1-yl)-2- | |
| | oxoethyl)-4-methyl-1H-pyrazol-3- | |
| | yl)amino)-N-(3-hydroxy-2,6- | |
| | dimethylphenyl)thiazole-5- | |
| | carboxamide | |
| BAA-606 | 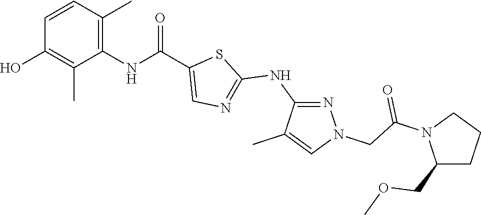 [SMILES: COC[C@@H]1CCCN1C(=O)Cn1cc(C)c(Nc2ncc(C(=O)Nc3c(C)ccc(O)c3C)s2)n1] | ¹H NMR (400 MHz, DMSO-d₆) δ 10.09 (s, 1H), 9.08 (s, 1H), 8.65 (s, 1H), 7.94 (s, 1H), 7.35 (d, J = 0.9 Hz, 1H), 6.83 (dt, J = 8.2, 0.8 Hz, 1H), 6.65 (d, J = 8.2 Hz, 1H), 4.82 (s, 2H), 4.13 (s, 1H), 3.55-3.46 (m, 1H), 3.41 (t, J = 7.1 Hz, 1H), 3.35-3.26 (m, 2H), 2.94 (d, J = 1.2 Hz, 3H), 2.05 (s, 3H), 1.99 (s, 3H), 1.98 (d, J = 0.8 Hz, 3H), 1.96-1.73 (m, 4H); MS (ES⁺) m/z 499.2 (M + H)⁺. |
| | (S)-N-(3-hydroxy-2,6-dimethylphenyl)- | |
| | 2-((1-(2-(2-(methoxymethyl)pyrrolidin- | |
| | 1-yl)-2-oxoethyl)-4-methyl-1H-pyrazol- | |
| | 3-yl)amino)thiazole-5-carboxamide | |
| BAA-607 | 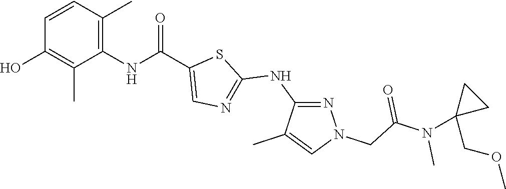 [SMILES: COCC1(N(C)C(=O)Cn2cc(C)c(Nc3ncc(C(=O)Nc4c(C)ccc(O)c4C)s3)n2)CC1] | ¹H NMR (400 MHz, DMSO-d₆) δ 10.09 (s, 1H), 9.06 (s, 1H), 8.65 (s, 1H), 7.94 (s, 1H), 7.33 (d, J = 0.9 Hz, 1H), 6.83 (d, J = 8.2 Hz, 1H), 6.65 (d, J = 8.1 Hz, 1H), 5.02 (s, 2H), 3.42 (s, 2H), 3.30 (s, 3H), 2.91-2.80 (m, 3H), 2.05 (s, 3H), 1.99 (s, 3H), 1.98 (d, J = 0.8 Hz, 3H), 1.04 (d, J = 71.1 Hz, 4H); MS (ES⁺) m/z 499.4 (M + H)⁺. |
| | N-(3-hydroxy-2,6-dimethylphenyl)-2- | |
| | ((1-(2-((1-(methoxymethyl)cyclopropyl) | |
| | (methylamino)-2-oxoethyl)-4-methyl- | |
| | 1H-pyrazol-3-yl)amino)thiazole-5- | |
| | carboxamide | |

### 2-(3-((5-((3-Hydroxy-2,6-dimethylphenyl)carbamoyl)thiazol-2-yl)amino)-4-methyl-1H-pyrazol-1-yl)-3-methoxypropanoic acid

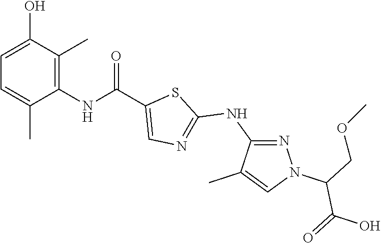 [SMILES: COCC(C(=O)O)n1cc(C)c(Nc2ncc(C(=O)Nc3c(C)ccc(O)c3C)s2)n1]

To a solution of methyl 2-[3-[5-[[3-[tert-butyl(dimethyl) silyl]oxy-2,6-dimethyl-phenyl]carbamoyl]thiazol-2-yl]amino]-4-methyl-pyrazol-1-yl]-3-methoxy-propanoate (86 mg, 0.150 mmol) in THF (1.5 mL) was added potassium trimethylsilanolate (75 μL, 0.150 mmol) 2M solution in THF. The resulting mixture was stirred at 50° C. for 16 h, and at rt for 16 h, recharging with potassium trimethylsilanolate (30 μL, 0.0600 mmol) twice during the duration. The reaction mixture was diluted with water (10 mL), acidified to pH 2 with 1M HCl aq., the aqueous layer extracted with CHCl3/IPA 4:1 (3×20 mL), dried over anhydrous sodium sulfate, filtered and evaporated to give the title product (46 mg, 0.0537 mmol, 35.82% yield) as a yellow solid. MS (ES+) m/z=446.2 [M+H]+.

### N-(3-Hydroxy-2.6-dimethylphenyl)-2-((1-(3-methoxy-1-oxo-1-(pyrrolidin-1-yl)propan-2-yl)-4-methyl-1H-pyrazol-3-yl)amino)thiazole-5-carboxamide( ) BAA-577

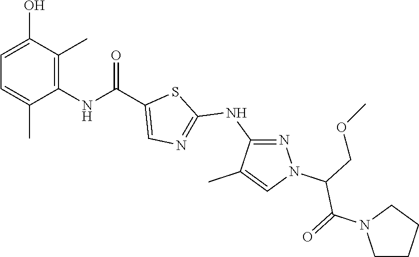 [SMILES: COCC(C(=O)N1CCCC1)n1cc(C)c(Nc2ncc(C(=O)Nc3c(C)ccc(O)c3C)s2)n1]

1-Propanephosphonic anhydride, 50% wt solution in EtOAc (92 μL, 0.155 mmol) was added dropwise (over 1 min) to a solution of 2-[3-[[5-[(3-hydroxy-2,6-dimethyl-phenyl) carbamoyl]thiazol-2-yl]amino]-4-methyl-pyrazol-1-yl]-3-methoxy-propanoic acid (46 mg, 0.103 mmol), pyrrolidine (8.5 μL, 0.103 mmol) and DIPEA (45 μL, 0.258 mmol) in THF (1 mL). The reaction mixture was heated to 40° C. and stirred for 18 h, and further 72 h at rt then the volatiles evaporated. The crude was purified by prep HPLC to afford the title compound (0.77 mg, 0.00147 mmol, 1.42% yield) as an off white solid.MS (ES+) m/z=499.3 [M+H]⁺, ¹H NMR (400 MHz, METHANOL-D4) δ 8.00 (s, 1H), 7.46 (d, J=1.0 Hz, 1H), 6.89 (d, J=8.2 Hz, 1H), 6.66 (d, J=8.2 Hz, 1H), 5.27 (dd, J=7.6, 6.1 Hz, 1H), 3.99-3.86 (m, 2H), 3.63 (dt, J=10.4, 6.7 Hz, 1H), 3.52-3.39 (m, 3H), 3.34 (s, 3H), 2.14 (s, 3H), 2.09 (s, 3H), 2.04 (d, J=0.8 Hz, 3H), 1.93 (q, J=6.3 Hz, 2H), 1.89-1.79 (m, 2H).

### N-(3-Hydroxy-2,6-dimethyl-phenyl)-2-[(1-methylpyrazolo[4,3-b]pyridin-3-yl)amino]thiazole-5-carboxamide (BAA-210)

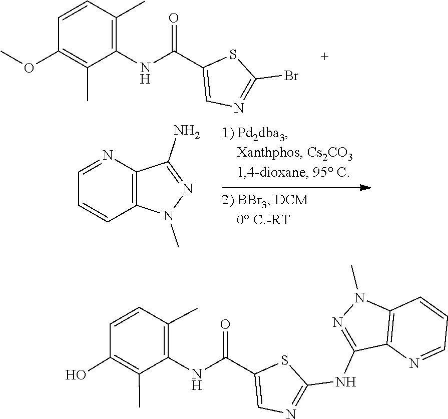

A mixture of 1-methylpyrazolo[4,3-b]pyridin-3-amine (230 mg, 1.55 mmol, 2.5 eq), 2-bromo-N-(3-methoxy-2,6-dimethyl-phenyl)thiazole-5-carboxamide (212 mg, 0.621 mmol, 1.0 eq), Pd₂dba₃
(85 mg, 0.0933 mmol, 0.15 eq), XantPhos (108 mg, 0.187 mmol, 0.3 eq) and Cs₂CO₃ (507 mg, 1.55 mmol, 2.5 eq) in 1,4-dioxane (12 mL) was degassed with nitrogen and stirred at 95° C. for 16 h. After cooling to RT, THF/Et₂O (1:1, 5 mL) was added and the suspension was sonicated for 30 s, filtered and washed with THF/Et₂O. The solids were redissolved in MeOH/DCM, filtered and concentrated in vacuo to afford the coupling product which was immediately dissolved in DCM (3.1 mL). BBr₃ (1M in DCM, 6.2 mL, 6.21 mmol, 10 eq) was added over 5 portions (2 eq each) over 20 h with stirring at RT (0° C. for initial addition). After complete conversion, MeOH(5 mL) was added slowly, and the mixture was stirred for a further 5 minutes before concentrating in vacuo. The crude mixture was purified by reverse phase chromatography (C18) eluting with 20-100% MeOH: H₂O followed by recrystallisation from MeOH/EtOAc to afford the title compound (55 mg, 0.139 mmol, 22%) as orange crystals. MS (ES+) m/z 395 (M+H). ¹H NMR (300 MHz, DMSO-d₆) δ 9.58 (s, 1H), 9.17 (br, 1H), 8.50 (m, 1H), 8.20 (s, 1H), 8.09 (m, 1H), 7.47 (dd, J=8.6, 4.3 Hz, 1H), 6.89 (d, J=8.2 Hz, 1H), 6.69 (d, J=8.2 Hz, 1H), 4.02 (s, 3H), 2.09 (s, 3H), 2.01 (s, 3H).

### Piperidinylmethyl Modifications from Phenol

### Amide Formation from N-(3-hydroxy-2,6-dimethyl-phenyl)-2-(4-piperidylmethylamino)thiazole-5-carboxamide hydrochloride BAA-079

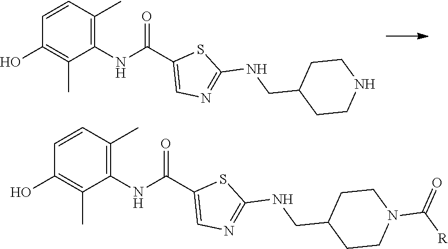

### N-(3-Hydroxy-2.6-dimethyl-phenyl)-2-[[1-(2-pyrrolidin-1-ylacetyl)-4-piperidyl]methylamino]thiazole-5-carboxamide (BAA-211)

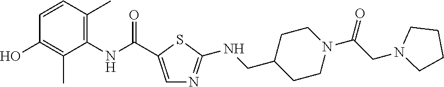 [SMILES: Cc1ccc(O)c(C)c1NC(=O)c1cnc(NCC2CCN(C(=O)CN3CCCC3)CC2)s1]

To methyl 2-morpholinoacetate (108 mg, 0.678 mmol, 1.0 eq) in water (0.1 mL) and THF (1 mL) was added lithium hydroxide monohydrate (31 mg, 0.746 mmol, 1.1 eq) and the reaction mixture was stirred at RT for 4 h, then concentrated in vacuo. This crude material was dissolved in DMF (1 mL) and this solution added to a mixture of N-(3-hydroxy-2,6-dimethyl-phenyl)-2-(4-piperidylmethylamino)thiazole-5-carboxamide hydrochloride (BAA-079)(90 mg, 0.228 mmol, 0.4 eq) and HATU (433 mg, 1.14 mmol, 2.0 eq) in DMF (4 mL), followed by DIPEA (0.40 mL, 2.28 mmol, 4.0 eq). The reaction was stirred at RT for 6 h. EtOAc (15 mL) and water (15 mL) were added. The phases were separated, and the aqueous phase extracted with EtOAc (2×20 mL). The combined organic phases were washed with water (5×10 mL), brine (20 mL), dried (MgSO₄), filtered and concentrated in vacuo to afford the title compound (1.8 mg, 0.00382 mmol, 0.67%) as a colourless powder. MS (ES+) m/z 472.4 (M+H). ¹H NMR (300 MHz, MeOD-d4) δ 8.53 (s, 2H), 7.86 (s, 1H), 6.90 (d, J=8.2 Hz, 1H), 6.67 (d, J=8.2 Hz, 1H), 4.53 (d, J=13.3 Hz, 1H), 4.37-4.19 (m, 2H), 3.80 (d, J=14.0 Hz, 1H), 3.32 (d, J=1.7 Hz, 6H), 3.11 (t, J=12.4 Hz, 1H), 2.80-2.65 (m, 1H), 2.15 (d, J=5.7 Hz, 3H), 2.08 (d, J=4.6 Hz, 6H), 1.89 (d, J=12.5 Hz, 2H), 1.49-1.09 (m, 2H).

### 2-[[1-(3,3-Dimethylbutanoyl)-4-piperidyl]methylamino]-N-(3-hydroxy-2,6-dimethyl-phenyl)thiazole-5-carboxamide (BAA-212)

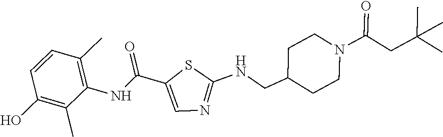 [SMILES: Cc1ccc(O)c(C)c1NC(=O)c1cnc(NCC2CCN(C(=O)CC(C)(C)C)CC2)s1]

To a mixture of N-(3-hydroxy-2,6-dimethyl-phenyl)-2-(4-piperidylmethylamino)thiazole-5-carboxamide hydrochloride (BAA-079)(30 mg, 0.0756 mmol, 1.0 eq) in DCM (4 mL) was added DIPEA (0.029 mL, 0.166 mmol, 2.2 eq) followed by tert-butylacetyl chloride (0.012 mL, 0.0831 mmol, 1.1 eq). The reaction mixture was stirred at RT for 16 h. DCM (5 mL) and water (5 mL) were added. The phases were separated, and the aqueous phase extracted with DCM (2×20 mL). The combined organic phases were washed with brine (10 mL), dried (MgSO₄), filtered and concentrated in vacuo. The crude material was purified by preparative HPLC-MS (high pH) to afford the title compound (7.6 mg, 0.0166 mmol, 22%) MS (ES+) m/z 459 (M+H). ¹H NMR (300 MHz, Methanol-d4) δ 7.86 (s, 1H), 6.90 (d, J=8.1, 0.7 Hz, 1H), 6.67 (d, J=8.2 Hz, 1H), 4.62 (d, J=13.3 Hz, 1H), 4.11 (d, J=13.8 Hz, 1H), 3.27 (d, J=6.7 Hz, 2H), 3.18-3.00 (m, 1H), 2.71-2.55 (m, 2H), 2.46-2.22 (m, 2H), 2.14 (d, J=0.7 Hz, 3H), 2.09 (s, 3H), 2.06-1.74 (m, 1H), 1.33-1.06 (m, 2H), 1.05-1.00 (m, 10H).

### N-(3-Hydroxy-2.6-dimethyl-phenyl)-2-[[1-[2-(4-methoxyphenyl)acetyl]-4-piperidyl]methylamino]thiazole-5-carboxamide (BAA-213)

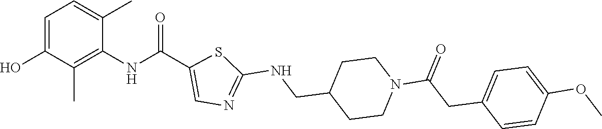 [SMILES: COc1ccc(CC(=O)N2CCC(CNc3ncc(C(=O)Nc4c(C)ccc(O)c4C)s3)CC2)cc1]

To a mixture of N-(3-hydroxy-2,6-dimethyl-phenyl)-2-(4-piperidylmethylamino)thiazole-5-carboxamide hydrochloride (BAA-079)(30 mg, 0.0756 mmol, 1.0 eq) in DCM (4 mL) was added DIPEA (0.029 mL, 0.166 mmol, 2.2 eq) followed by 4-methoxyphenylacetyl chloride (0.016 mL, 0.0831 mmol, 1.1 eq). The reaction mixture was stirred at RT for 16 h. DCM (5 mL) and water (5 mL) were added. The phases were separated, and the aqueous phase extracted with DCM (2×5 mL). The combined organic phases were washed with brine (10 mL), dried (MgSO₄), filtered and concentrated in vacuo. The crude material was purified by preparative HPLC-MS (high pH) to afford the title compound (4.0 mg, 0.00786 mmol, 10%). MS (ES+) m/z 509 (M+H). ¹H NMR (300 MHz, Methanol-d4) δ 7.85 (s, 1H), 7.21-7.11 (m, 2H), 6.94-6.83 (m, 3H), 6.67 (d, J=8.2 Hz, 1H), 4.68-4.51 (m, 1H), 4.04 (d, J=14.0 Hz, 1H), 3.77 (s, 3H), 3.71 (d, J=2.8 Hz, 2H), 3.20 (d, J=6.7 Hz, 2H), 3.12-2.93 (m, 1H), 2.74-2.56 (m, 1H), 2.14 (s, 3H), 2.09 (s, 3H), 2.02-1.60 (m, 3H), 1.21-0.79 (m, 2H).

### N-(3-Hydroxy-2,6-dimethyl-phenyl)-2-[[1-[2-(5-methoxy-2-pyridyl)acetyl]-4-piperidyl]methylamino]thiazole-5-carboxamide (BAA-100)

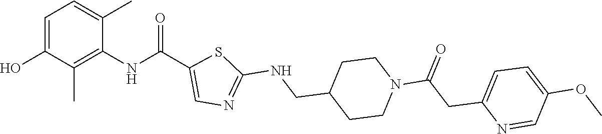 [SMILES: COc1ccc(CC(=O)N2CCC(CNc3ncc(C(=O)Nc4c(C)ccc(O)c4C)s3)CC2)nc1]

To a mixture of N-(3-hydroxy-2,6-dimethyl-phenyl)-2-(4-piperidylmethylamino)thiazole-5-carboxamide; hydrochloride (BAA-079)(42 mg, 0.106 mmol, 1.00 eq), 2-(5-methoxy-2-pyridyl) acetic acid (19 mg, 0.116 mmol, 1.10 eq) and 2-(7-aza-1H-benzotriazole-1-yl)-1,1,3,3-tetramethyluronium hexafluorophosphate (HATU)(44 mg, 0.116 mmol, 1.10 eq) in isopropanol (IPA)(4 mL) was added N,N-diisopropylethylamine (0.059 mL, 0.339 mmol, 3.20 eq). The reaction mixture was stirred at RT for 16 h then concentrated in vacuo. DCM (5 mL) and water (5 mL) were added. The phases were separated and the aqueous phase extracted with DCM (2×5 mL). Combined organic phases were washed with brine (10 mL), dried over MgSO₄, filtered and concentrated in vacuo. Reverse phase column chromatography (water:acetonitrile) gave the title compound (17 mg, 0.0334 mmol, 32%). MS (ES+) m/z 510 (M+H). ¹H NMR (300 MHz, Methanol-d4) δ 8.15 (dd, J=3.0, 0.7 Hz, 1H), 7.86 (s, 1H), 7.38 (dd, J=8.6, 3.0 Hz, 1H), 7.29 (dd, J=8.7, 0.7 Hz, 1H), 6.90 (dt, J=8.2, 0.7 Hz, 1H), 6.67 (d, J=8.2 Hz, 1H), 4.56 (d, J=13.3 Hz, 1H), 4.12 (d, J=11.1 Hz, 1H), 3.89 (d, J=3.2 Hz, 1H), 3.86 (s, 3H), 3.24 (d, J=6.7 Hz, 2H), 3.19-3.01 (m, 1H), 2.72-2.62 (m, 2H), 2.14 (s, 3H), 2.09 (s, 3H), 2.00-1.91 (m 1H), 1.85-1.76 (m, 2H), 1.22-1.01 (m, 2H).

### Piperidinylmethyl Modifications from Phenol

### Alkyation and Acylation with N-(3-methoxy-2,6-dimethyl-phenyl)-2-(4-piperidylmethylamino)thiazole-5-carboxamide and subsequent de-methylation

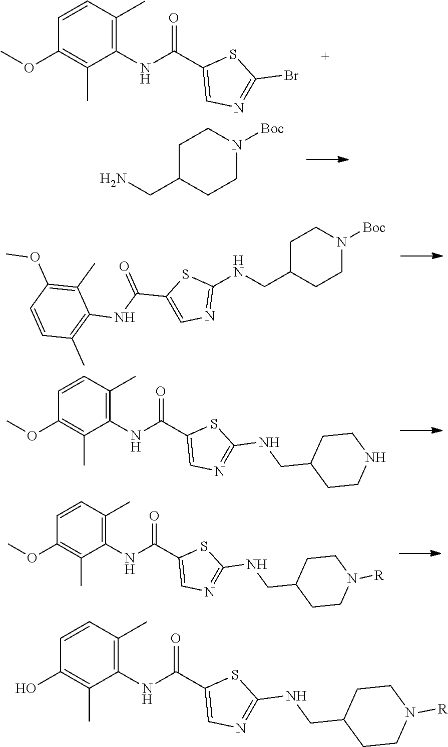

### N-(3-Methoxy-2,6-dimethyl-phenyl)-2-(4-piperidylmethylamino)thiazole-5-carboxamide hydrochloride

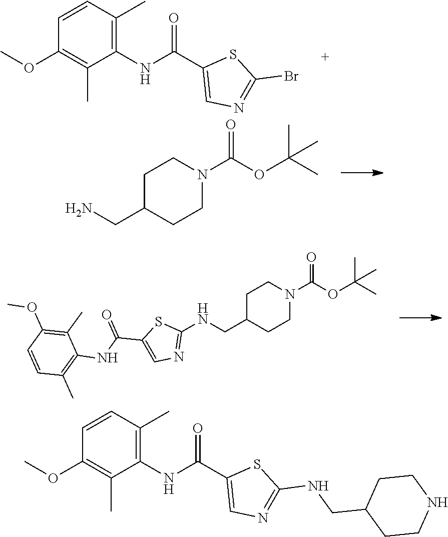

Step 1: To 2-bromo-N-(3-methoxy-2,6-dimethyl-phenyl)thiazole-5-carboxamide (314 mg, 0.920 mmol, 1.0 eq) in THF (6 mL) at RT was added 4-(aminomethyl)-1-N-Boc-piperidine (0.39 mL, 1.84 mmol, 1.0 eq) and the reaction mixture stirred under microwave irradiation at 60° C. for 12 h. Et₂O (15 mL) and water (15 mL) were added. The phases were separated, and the aqueous phase extracted with Et₂O (2×20 mL). The combined organic phases were washed with brine (20 mL), dried (MgSO₄), filtered and concentrated in vacuo. The resulting residue was chromatographed (SiO₂) eluting with 0-50% EtOAc: PE to afford tert-butyl 4-[5-[(3-methoxy-2,6-dimethyl-phenyl) carbamoyl]thiazol-2-yl]amino]methyl]piperidine-1-carboxylate (253 mg, 0.533 mmol, 58%). MS (ES+) m/z 475 (M+H). ¹H NMR (300 MHz, Methanol-d4) δ 7.87 (s, 1H), 7.12-6.97 (m, 1H), 6.83 (d, J=8.4 Hz, 1H), 4.10 (d, J=13.5 Hz, 2H), 3.81 (s, 3H), 3.25 (d, J=6.7 Hz, 2H), 2.77 (s, 2H), 2.17 (d, J=0.7 Hz, 3H), 2.09 (s, 3H), 1.99-1.70 (m, 2H), 1.46 (s, 9H), 1.31-1.01 (m, 3H).

Step 2: Prepared as described in Method E from tert-butyl 4-[[5-[(3-methoxy-2,6-dimethyl-phenyl) carbamoyl]thiazol-2-yl]amino]methyl]piperidine-1-carboxylate (253 mg, 0.533 mmol, 1.0 eq) in HCl (4M in 1,4-dioxane, 4 mL, 16 mmol, 30 eq) for 4 h at RT to afford N-(3-methoxy-2,6-dimethyl-phenyl)-2-(4-piperidylmethylamino)thiazole-5-carboxamide hydrochloride (217 mg, 0.528 mmol, 99%) which was used directly without purification. MS (ES+) m/z 375 (M+H).

### Amide Formations from N-(3-methoxy-2,6-dimethyl-phenyl)-2-(4-piperidylmethylamino)thiazole-5-carboxamide

### 2-[[1-[2-(4-Chlorophenyl)acetyl]-4-piperidyl]methylamino]-N-(3-methoxy-2,6-dimethyl-phenyl)thiazole-5-carboxamide

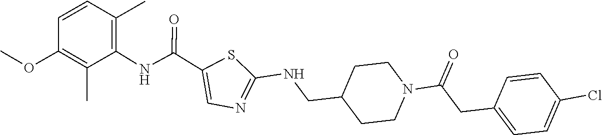 [SMILES: COc1ccc(C)c(NC(=O)c2cnc(NCC3CCN(C(=O)Cc4ccc(Cl)cc4)CC3)s2)c1C]

To N-(3-methoxy-2,6-dimethyl-phenyl)-2-(4-piperidylmethylamino)thiazole-5-carboxamide hydrochloride (60 mg, 0.146 mmol, 1.0 eq) and DIPEA (0.051 mL, 0.292 mmol, 2.0 eq) in DCM (3 mL) at RT was added dropwise p-chlorophenylacetyl chloride (0.021 mL, 0.146 mmol, 1.0 eq) and stirred for 16 h. DCM (5 mL) and water (5 mL) were added. The phases were separated, and the aqueous phase extracted with DCM (2×5 mL). The combined organic phases were washed with aq. NH₄Cl (10% w/w, 10 mL), brine (20 mL), dried (MgSO₄), filtered and concentrated in vacuo. The resulting residue was chromatographed (SiO₂) eluting with 0-20% EtOAc: PE to afford the title compound (37 mg, 0.0702 mmol, 48%) and used directly in the next step. MS (ES+) m/z 526.9/528.9 (M+H), CI isotope pattern.

### 2-[[1-(3-Hydroxy-3-methyl-butanoyl)-4-piperidyl]methylamino]-N-(3-methoxy-2,6-dimethyl-phenyl)thiazole-5-carboxamide

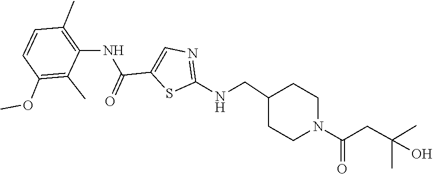 [SMILES: COc1ccc(C)c(NC(=O)c2cnc(NCC3CCN(C(=O)CC(C)(C)O)CC3)s2)c1C]

To a mixture of N-(3-methoxy-2,6-dimethyl-phenyl)-2-(4-piperidylmethylamino)thiazole-5-carboxamide; hydrochloride (60 mg, 0.146 mmol, 1.0 eq), HATU (56 mg, 0.146 mmol, 1.0 eq) and 3-hydroxy-3-methylbutanoic acid (0.016 mL, 0.146 mmol, 1.0 eq) in IPA (3 mL) was added DIPEA
(0.13 mL, 0.730 mmol, 5.0 eq) and the reaction mixture stirred for 16 h at RT before concentrating in vacuo. DCM (5 mL) and water (5 mL) were added. The phases were separated, and the aqueous phase extracted with DCM (2×5 mL). The combined organic phases were washed with aq. NH₄Cl (10% w/w, 10 mL), brine (20 mL), dried (MgSO₄), filtered and concentrated in vacuo. The resulting residue was chromatographed (C18) eluting with 5-95% MeCN: water to afford title compound (42 mg, 0.0876 mmol, 60%) which was used directly in the next step. MS (ES+) m/z 475.3 (M+H),

### N-(3-Methoxy-2,6-dimethyl-phenyl)-2-[[1-(2-morpholinoacetyl)-4-piperidyl]methylamino]thiazole-5-carboxamide

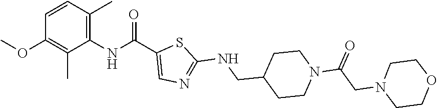 [SMILES: COc1ccc(C)c(NC(=O)c2cnc(NCC3CCN(C(=O)CN4CCOCC4)CC3)s2)c1C]

To N-(3-methoxy-2,6-dimethyl-phenyl)-2-(4-piperidylmethylamino)thiazole-5-carboxamide hydrochloride (110 mg, 0.268 mmol, 1.0 eq) and HATU (251 mg, 0.661 mmol, 2.5 eq) in DMF (4 mL) was added a solution of (2-morpholinoacetyl)oxylithium (prepared as described in International Patent Publication WO 2006/138350 A2)(100 mg, 0.662 mmol, 2.5 eq) in DMF (1 mL), followed by DIPEA (0.33 mL, 1.87 mmol, 7.00 eq). The mixture was stirred at RT for 6 h. EtOAc (15 mL) and water (15 mL) were added. The phases were separated, and the aqueous phase extracted with EtOAc (2×20 mL). The combined organic phases were washed with water (5×10 mL), brine (20 mL), dried (MgSO₄), filtered and concentrated in vacuo to afford title compound (20 mg, 0.0399 mmol, 15%) which was used directly in the next step. MS (ES+) m/z 502.3 (M+H).

### Alkylation of N-(3-methoxy-2,6-dimethyl-phenyl)-2-(4-piperidylmethylamino)thiazole-5-carboxamide 2-[[1-[2-(Dimethylamino)-2-oxo-ethyl]-4-piperidyl]methylamino]-N-(3-methoxy-2,6-dimethyl-phenyl)thiazole-5-carboxamide

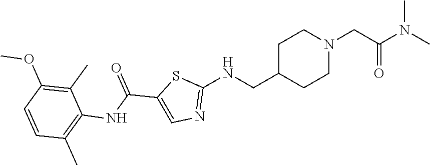

To N-(3-methoxy-2,6-dimethyl-phenyl)-2-(4-piperidylmethylamino)thiazole-5-carboxamide hydrochloride (60 mg, 0.146 mmol, 1.0 eq) and DIPEA (0.051 mL, 0.292 mmol, 2.0 eq) in DCM (2 mL) at RT was added dropwise 2-bromo-N,N-dimethylacetamide (0.017 mL, 0.153 mmol, 1.05 eq) and the reaction mixture was stirred for 16 h. DCM (5 mL) and water (5 mL) were added. The phases were separated, and the aqueous phase extracted with DCM (2×5 mL). The combined organic phases were washed with aq. NH₄Cl (10% w/w, 10 mL), brine (20 mL), dried (MgSO₄), filtered and concentrated in vacuo. The resulting residue was chromatographed (C18) eluting with 5-95% MeCN: water to afford the title compound (31 mg, 0.0679 mmol, 46%) which was used directly in the next step. MS (ES+) m/z 460.3 (M+H).

Methoxy Deprotection with BBr₃

The following example compounds were prepared similarly using Method K from their methoxy precursors. Purification was achieved by preparative HPLC-MS using a gradient of high or low pH aq. MeCN.

| # | Structure & Name | Analytical Data |
|---|---|---|
| BAA-214 | 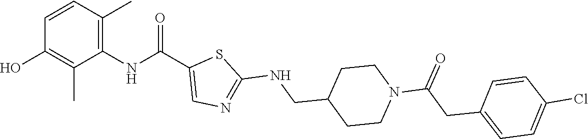 [SMILES: Cc1ccc(O)c(C)c1NC(=O)c1cnc(NCC2CCN(C(=O)Cc3ccc(Cl)cc3)CC2)s1] 2-[[1-[2-(4-chlorophenyl)acetyl]-4- piperidyl]methylamino]-N-(3-hydroxy-2,6- dimethyl-phenyl)thiazole-5-carboxamide | MS (ES+) m/z 513/515 (M + H), Cl isotope pattern. ¹H NMR (300 MHz, Methanol- d₄) δ 7.85 (s, 1H), 7.37-7.29 (m, 2H), 7.28-7.18 (m, 2H), 6.90 (dt, J = 8.3, 0.7 Hz, 1H), 6.67 (d, J = 8.2 Hz, 1H), 4.57 (d, J = 13.4 Hz, 1H), 4.03 (d, J = 13.9 Hz, 1H), 3.77 (s, 2H), 3.23 (d, J = 6.7 Hz, 2H), 3.16-2.96 (m, 1H), 2.77-2.57 (m, 1H), 2.14 (d, J = 0.6 Hz, 3H), 2.09 (s, 3H), 2.03-1.65 (m, 3H), 1.23- 0.87 (m, 2H). |
| BAA-215 | 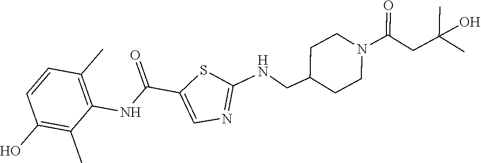 [SMILES: Cc1ccc(O)c(C)c1NC(=O)c1cnc(NCC2CCN(C(=O)CC(C)(C)O)CC2)s1] | MS (ES+) m/z 461 (M + H). ¹H NMR (300 MHz, Methanol- d₄) δ 7.87 (s, 1H), 6.90 (dt, J = 8.3, 0.7 Hz, 1H), 6.67 (d, J = 8.2 Hz, 1H), 4.61 (d, J = 13.3 Hz, 1H), 4.11 (d, J = 13.7 Hz, 1H), 3.27 (d, J = 6.8 Hz, 2H), 3.21-2.99 (m, 1H), 2.74-2.49 (m, 3H), 2.14 (s, 3H), 2.09 (s, 3H), 2.07-1.73 (m, 3H), 1.28 (s, 8H). |
| | N-(3-hydroxy-2,6-dimethyl-phenyl)-2-[[1- | |
| | (3-hydroxy-3-methyl-butanoyl)-4- | |
| | piperidyl]methylamino]thiazole-5- | |
| | carboxamide | |
| BAA-216 | 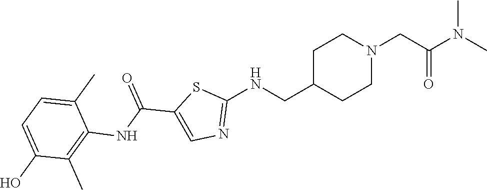 [SMILES: Cc1ccc(O)c(C)c1NC(=O)c1cnc(NCC2CCN(CC(=O)N(C)C)CC2)s1] | MS (ES+) m/z 446 (M + H). ¹H NMR (300 MHz, Methanol- d₄) δ 8.50 (s, 2H), 7.87 (s, 1H), 6.90 (dt, J = 8.1, 0.7 Hz, 1H), 6.68 (d, J = 8.2 Hz, 1H), 3.87 (s, 2H), 3.48-3.35 (m, 2H), 3.03 (s, 3H), 2.98 (s, 3H), 2.77 (t, J = 12.0 Hz, 2H), 2.14 (s, 3H), 2.09 (s, 3H), 2.05-1.80 (m, 3H), 1.56 (q, J = 13.0 Hz, 2H). |
| | 2-[[1-[2-(dimethylamino)-2-oxo-ethyl]-4- | |
| | piperidyl]methylamino]-N-(3-hydroxy-2,6- | |
| | dimethyl-phenyl)thiazole-5-carboxamide | |
| BAA-217 | 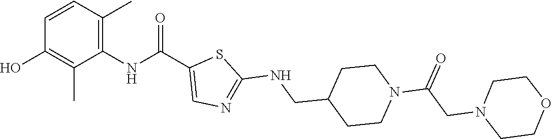 [SMILES: Cc1ccc(O)c(C)c1NC(=O)c1cnc(NCC2CCN(C(=O)CN3CCOCC3)CC2)s1] N-(3-hydroxy-2,6-dimethyl-phenyl)-2-[[1- (2-morpholinoacetyl)-4- piperidyl]methylamino]thiazole-5-carboxamide | MS (ES+) m/z 488 (M + H). ¹H NMR (300 MHz, Methanol- d₄) δ 7.89 (s, 1H), 6.92 (d, J = 8.2 Hz, 1H), 6.69 (d, J = 8.2 Hz, 1H), 4.54 (d, J = 13.3 Hz, 1H), 4.17 (d, J = 13.7 Hz, 1H), 3.72 (t, J = 4.7 Hz, 4H), 3.33 (dt, J = 3.3, 1.6 Hz, 5H), 3.19 (d, J = 14.0 Hz, 1H), 3.13-2.60 (m, 1H), 2.54 (t, J = 4.7 Hz, 4H), 2.16 (s, 3H), 2.11 (s, 3H), 1.88 (t, J = 12.5 Hz, 2H), 1.46-0.98 (m, 2H). |

### Pd Coupling of 2-chloro-N-(3-hydroxy-2,6-dimethyl-phenyl)thiazole-5-carboxamide with amines

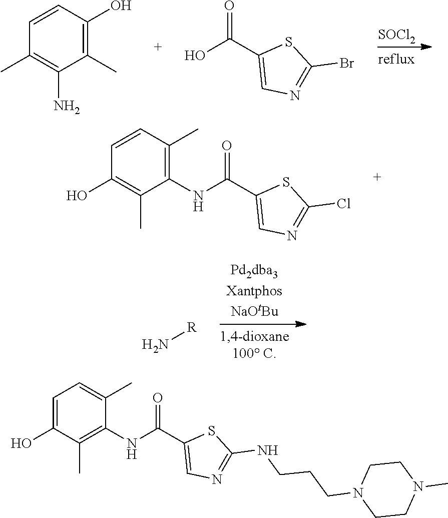

### N-(3-Hydroxy-2,6-dimethyl-phenyl)-2-[3-(4-methylpiperazin-1-yl)propylamino]thiazole-5-carboxamide (BAA-218)

Step 1:2-bromo-1,3-thiazole-5-carboxylic acid (650 mg, 3.12 mmol, 1.0 eq) was dissolved in SOCl₂ (10 mL, 3.12 mmol, 1.0 eq) at reflux for 1 h. After cooling to RT, the mixture was concentrated in vacuo, using toluene as a co-evaporant (x 3). The crude material was dissolved in DCM (7 mL), cooled to 0° C., pyridine (0.53 mL, 6.56 mmol, 2.1 eq) followed by 3-amino-2,4-dimethyl-phenol (480 mg, 3.50 mmol, 1.12 eq) was added in one portion. The reaction mixture was allowed to warm to RT slowly overnight. The reaction was quenched by the addition of water. The aqueous phase was extracted with DCM (2×20 mL). The combined organic phases were washed with aq. NH₄Cl (2×10 mL), brine (15 mL), dried (MgSO₄), filtered and concentrated in vacuo. The resulting residue was chromatographed (SiO₂) eluting with 0-80% EtOAc: PE to afford 2-chloro-N-(3-hydroxy-2,6-dimethyl-phenyl)thiazole-5-carboxamide (486 mg, 1.72 mmol, 55%). MS (ES+) m/z 283/285 (M+H), CI isotope pattern. ¹H NMR (300 MHz, Methanol-d4) δ 8.29 (s, 1H), 7.04-6.83 (m, 1H), 6.71 (d, J=8.2 Hz, 1H), 2.21-2.05 (m, 6H).

Step 2: Prepared as described in Method C from 2-chloro-N-(3-hydroxy-2,6-dimethyl-phenyl)thiazole-5-carboxamide (108 mg, 0.382 mmol, 1.0 eq), 3-(4-methyl-piperazin-1-yl)-propylamine (0.13 mL, 0.764 mmol, 2.0 eq), sodium tert-butoxide (147 mg, 1.53 mmol, 4.0 eq), Pd₂dba₃ (35 mg, 0.0382 mmol, 0.10 eq) and XantPhos (22 mg, 0.0382 mmol, 0.10 eq) in 1,4-dioxane (4 mL) to return the title compound (2.4 mg, 0.00595 mmol, 1.6%) after preparative HPLC-MS (high pH). MS (ES+) m/z 404.3 (M+H). ¹H NMR (300 MHz, Methanol-d4) δ 9.19 (s, 1H), 8.57 (s, 1H), 7.87 (s, 1H), 7.00-6.86 (m, 1H), 6.69 (t, J=9.3 Hz, 1H), 3.40 (t, J=6.7 Hz, 2H), 2.52 (td, J=6.9, 4.1 Hz, 10H), 2.39 (s, 3H), 2.14 (d, J=0.7 Hz, 3H), 2.09 (s, 3H), 1.86 (dq, J=14.1, 6.8 Hz, 2H).

### Modification of the Thiazole Scaffold

### Amino Thiazole. Synthesis of Example BAA-219

### 4-Amino-N-(3-hydroxy-2,6-dimethyl-phenyl)-2-[(1-methylpyrazol-3-yl)amino]thiazole-5-carboxamide (BAA-219)

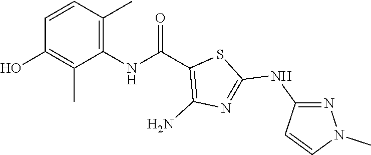 [SMILES: Cc1ccc(O)c(C)c1NC(=O)c1sc(Nc2ccn(C)n2)nc1N]

Step 1: To a solution of 3-methoxy-2,6-dimethyl-aniline (150 mg, 0.992 mmol, 1.0 eq) and NEt₃ (0.17 mL, 1.19 mmol, 1.2 eq) in anhydrous DCM (1 mL) at 0° C. was added bromoacetyl bromide (0.10 mL, 1.19 mmol, 1.2 eq) dropwise. The mixture was stirred for 0.25 h, warmed to RT and stirred for a further 0.75 h. The reaction mixture was diluted with DCM (5 mL), water (5 mL) added and the phases separated. The aqueous phase was washed with DCM (5 mL), and the organics combined. The organics were washed with brine (10 mL), dried (MgSO₄), filtered and concentrated in vacuo. The resulting residue was chromatographed (SiO₂) eluting with 0-50% EtOAc: PE to afford 2-bromo-N-(3-methoxy-2,6-dimethyl-phenyl)acetamide (201 mg, 0.739 mmol, 74%) as an off white powder. MS (ES+) m/z 272.1/274.1 (M+H), Br isotope pattern. ¹H NMR (300 MHz, Chloroform-d) δ 7.74 (s, 1H), 7.05 (d, J=8.4 Hz, 1H), 6.76 (d, J=8.4 Hz, 1H), 4.09 (s, 2H), 3.81 (s, 3H), 2.18 (s, 3H), 2.11 (s, 3H).

Step 2: To a mixture of 3-isothiocyanato-1-methyl-1H-pyrazole (54 mg, 0.386 mmol, 1.05 eq) and cyanamide (16 mg, 0.386 mmol, 1.05 eq) in THF (1.5 mL) was added potassium tert-butoxide (95 mg, 0.845 mmol, 2.3 eq) under nitrogen and stirred for 0.25 h. 2-bromo-N-(3-methoxy-2,6-dimethyl-phenyl)acetamide (100 mg, 0.367 mmol, 1.0 eq) was added and the mixture stirred for 2 h. The reaction mixture was concentrated in vacuo, water (5 mL) added, stirred for 0.5 h and filtered. The solid was washed with water (2×10 mL). The aqueous phase was acidified with aq. HCl (2M) and extracted with EtOAc (3×10 mL). The extracts were dried (MgSO₄), filtered and concentrated in vacuo. The resulting solid was chromatographed (SiO₂) eluting with 50-100% EtOAc: PE followed by triturated with Et₂O, to afford 4-amino-N-(3-methoxy-2,6-dimethyl-phenyl)-2-[(1-methylpyrazol-3-yl)amino]thiazole-5-carboxamide (17 mg, 0.0456 mmol, 12%) as an orange powder. MS (ES+) m/z 373.2 (M+H). ¹H NMR (300 MHz, DMSO-d₆) δ 10.82 (s, 1H), 8.46 (s, 1H), 7.61 (d, J=2.3 Hz, 1H), 7.01 (d, J=8.4 Hz, 1H), 6.87-6.66 (m, 3H), 6.01 (d, J=2.2 Hz, 1H), 3.76 (s, 6H), 2.09 (s, 3H), 1.99 (s, 3H).

Step 3: To a suspension of 4-amino-N-(3-methoxy-2,6-dimethyl-phenyl)-2-[(1-methylpyrazol-3-yl)amino]thiazole-5-carboxamide (14 mg, 0.0376 mmol, 1.0 eq) in DCM (2 mL) was added BBr₃ (1M in DCM, 0.038 mL, 0.0376 mmol, 1.0 eq) and the mixture stirred for 1.5 h. Water (5 mL) was added and the mixture stirred for 0.5 h. MeOH added until full dissolution then the reaction mixture was extracted with EtOAc (3×10 mL). The mixture was concentrated in vacuo followed by chromatography purification (SiO₂) eluting with 0-5% MeOH(+10% aq. NH₃): DCM to afford the title compound (8.0 mg, 0.0223 mmol, 59%) as a light orange powder. MS (ES+) m/z 358.9 (M+H). ¹H NMR (300 MHz, DMSO-d₆) δ 10.79 (s, 1H), 9.05 (s, 1H), 8.37 (s, 1H), 7.60 (d, J=2.2 Hz, 1H), 6.83 (d, J=8.2 Hz, 1H), 6.74 (s, 2H), 6.63 (d, J=8.1 Hz, 1H), 6.02 (d, J=2.3 Hz, 1H), 3.76 (s, 3H), 2.04 (s, 3H), 1.96 (s, 3H).

### Oxazole Scaffold Analogue

### N-(3-Hydroxy-2,6-dimethylphenyl)-2-((1-methyl-1H-pyrazol-3-yl)amino) oxazole-5-carboxamide ( ) BAA-462

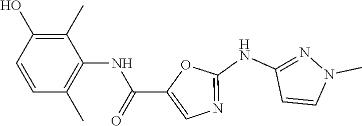 [SMILES: Cc1ccc(O)c(C)c1NC(=O)c1cnc(Nc2ccn(C)n2)o1]

Step 1: Ethyl 2-((1-methyl-1H-pyrazol-3-yl)amino) oxazole-5-carboxylate

A mixture of ethyl 2-bromooxazole-5-carboxylate (250 mg, 1.14 mmol), 1-methyl-1H-pyrazol-3-ylamine (132 mg, 1.36 mmol), cesium carbonate (745 mg, 2.27 mmol), tris(dibenzylideneacetone) dipalladium (0)(104 mg, 0.114 mmol) and 4,5-bis(diphenylphospheno)-9,9-dimethylxanthene (131 mg, 0.227 mmol) in 1,4-dioxane (11 mL) was degassed under nitrogen then stirred at 100° C. overnight, cooled down and evaporated to dryness. The residue was suspended in aq. NaHCO₃ (20 mL) and extracted with EtOAc (3×20 mL) and the combined organics washed with brine (20 mL), dried over Na₂SO₄, filtered and evaporated. This residue was purified by flash chromatography (gradient of MeOH 0-15% in DCM) to give a residue used in the next step without further purification. MS (ES+) m/z 237.2 (M+H).

Step 2:2-((1-Methyl-1H-pyrazol-3-yl)amino) oxazole-5-carboxylic acid

A suspension of ethyl 2-((1-methyl-1H-pyrazol-3-yl)amino) oxazole-5-carboxylate (365 mg, 1.55 mmol) in methanol (5 mL) and water (2.5 mL) was reacted with sodium hydroxide (95 mg, 2.32 mmol) at rt for 2 hrs then concentrated under reduced pressure and diluted with H₂O (15 mL). This aq. phase was washed with DCM (15 mL) and Et₂O (15 mL) then acidified with aq. HCl 2N
(10 mL) and extracted with EtOAc (6×25 mL). The combined organics were dried over anhydrous MgSO₄, filtered and evaporated to dryness to give a residue used in the next step without further purification. MS (ES+) m/z 209.1 (M+H).
   Step 3: N-(3-Methoxy-2,6-dimethylphenyl)-2-((1-methyl-1H-pyrazol-3-yl)amino) oxazole-5-carboxamide

A solution of 2-((1-methyl-1H-pyrazol-3-yl)amino) oxazole-5-carboxylic acid (158 mg, 0.759 mmol) and 3-methoxy-2,6-dimethyl-aniline (126 mg, 0.835 mmol) in THF (5 mL) and N,N-diisopropylethylamine (0.26 mL, 1.52 mmol) was reacted with 1-propanephosphonic anhydride 50% in ethyl acetate (0.67 mL, 1.14 mmol) at 70° C. for 20 hrs, then cooled down, concentrated under reduced pressure and diluted with H₂O. This aq. phase was extracted with EtOAc and the combined organic layers were washed with brine, dried over Na₂SO₄, filtered and evaporated to dryness. This residue was purified by flash chromatography (gradient of MeOH 0-10% in DCM) to give a residue used in the next step without further purification. MS (ES+) m/z 342.3 (M+H).

Step 4: N-(3-Hydroxy-2,6-dimethylphenyl)-2-((1-methyl-1H-pyrazol-3-yl)amino) oxazole-5-carboxamide

A solution of N-(3-methoxy-2,6-dimethylphenyl)-2-((1-methyl-1H-pyrazol-3-yl)amino) oxazole-5-carboxamide (68 mg, 0.199 mmol) in DCM (2 mL) was reacted with boron tribromide solution (1M in DCM, 0.60 mL, 0.597 mmol) at rt for 3 hrs, then quenched with aq. NaHCO₃ (10 mL), stirred at rt for 15 min and extracted with EtOAc (3×10 mL). The combined organics were washed with brine (10 mL), dried over Na₂SO₄, filtered and evaporated to dryness. The residue was purified by preparative HPLC(low pH) to give N-(3-hydroxy-2,6-dimethyl-phenyl)-2-[(1-methylpyrazol-3-yl)amino]oxazole-5-carboxamide (14 mg, 0.0367 mmol, 18%) as a formic acid salt. MS (ES+) m/z 328.3 (M+H); 1H-NMR (300 MHz, MeOD) δ 8.46 (s, 1H), 7.68 (s, 1H), 7.51 (d, J=2.3 Hz, 1H), 6.95 (dt, J=8.2, 0.7 Hz, 1H), 6.72 (d, J=8.2 Hz, 1H), 6.43 (d, J=2.4 Hz, 1H), 3.84 (s, 3H), 2.18 (s, 3H), 2.13 (s, 3H).

### Thiadiazole Scaffold Analogue

### N-(3-Hydroxy-2,6-dimethylphenyl)-5-((1-methyl-1H-pyrazol-3-yl)amino)-1,3,4-thiadiazole-2-carboxamide( ) BAA-463

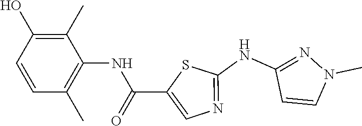 [SMILES: Cc1ccc(O)c(C)c1NC(=O)c1cnc(Nc2ccn(C)n2)s1]

Step 1: Ethyl 5-((1-methyl-1H-pyrazol-3-yl)amino)-1,3,4-thiadiazole-2-carboxylate.

A mixture of ethyl 5-bromo-1,3,4-thiadiazole-2-carboxylate (500 mg, 2.11 mmol), 1-methyl-1H-pyrazol-3-ylamine (0.25 g, 2.53 mmol), cesium carbonate (1.38 g, 4.22 mmol), tris(dibenzylideneacetone) dipalladium (0)(0.12 mL, 0.211 mmol) and 4,5-bis(diphenylphospheno)-9,9-dimethylxanthene (244 mg, 0.422 mmol) in 1,4-dioxane (21 mL) was degassed under nitrogen then stirred at 100° C. overnight. After cooling down to rt, the mixture was filtered through a dicalite pad and evaporated to dryness. The resulting residue was purified by flash chromatography (gradient of MeOH 0-10% in DCM) to give a residue used in the next step without further purification. MS (ES+) m/z 254.5 (M+H).

Step 2:5-((1-Methyl-1H-pyrazol-3-yl)amino)-1,3,4-thiadiazole-2-carboxylic acid

A solution of ethyl 5-((1-methyl-1H-pyrazol-3-yl)amino)-1,3,4-thiadiazole-2-carboxylate (335 mg, 1.32 mmol) in methanol (5 mL) and water (2.5 mL) was reacted with sodium hydroxide (81 mg, 1.98 mmol) at rt for 2 hrs then filtered, concentrated under reduced pressure and made acidic by addition of aqueous HCl 2N. The resulting precipitate was filtered, washed with water and ether to give a residue used in the next step without further purification. MS (ES+) m/z 226.5 (M+H).

Step 3: N-(3-Methoxy-2,6-dimethylphenyl)-5-((1-methyl-1H-pyrazol-3-yl)amino)-1,3,4-thiadiazole-2-carboxamide

To a solution of 3-methoxy-2,6-dimethyl-aniline (129 mg, 0.855 mmol) and 5-[(1-methylpyrazol-3-yl)amino]-1,3,4-thiadiazole-2-carboxylic acid (175 mg, 0.777 mmol) in THF (5 mL) were added N, N-diisopropylethylamine (0.27 mL, 1.55 mmol) and 1-propanephosphonic anhydride (50% in ethyl acetate, 0.69 mL, 1.17 mmol). The mixture was stirred at 65° C. for 20 hrs then cooled down to rt and evaporated to dryness. The residue was then suspended in H₂O (10 mL) and extracted with EtOAc (3×10 mL). The combined organics were washed with brine, dried over Na₂SO₄, filtered, and evaporated. The mixture was purified by flash chromatography (gradient of MeOH 0-10% in DCM) to give a residue used in the next step without further purification. MS (ES+) m/z 359.3 (M+H).

Step 4: N-(3-Hydroxy-2,6-dimethylphenyl)-5-((1-methyl-1H-pyrazol-3-yl)amino)-1,3,4-thiadiazole-2-carboxamide

A solution of N-(3-methoxy-2,6-dimethylphenyl)-5-((1-methyl-1H-pyrazol-3-yl)amino)-1,3,4-thiadiazole-2-carboxamide (155 mg, 0.432 mmol) in DCM (4.3 mL) was reacted with boron tribromide solution (1M in DCM, 1.1 mL, 1.08 mmol) at rt for 3 hrs, then quenched with aq. NaHCO₃ (10 mL), stirred at rt for 15 min and extracted with EtOAc (3×10 mL). The combined organics were washed with brine (20 mL), dried over Na₂SO₄, filtered and evaporated to dryness. The residue was purified by flash chromatography (gradient of MeOH 0-15% in DCM) followed by preparative HPLC(low pH) to give N-(3-hydroxy-2,6-dimethylphenyl)-5-((1-methyl-1H-pyrazol-3-yl)amino)-1,3,4-thiadiazole-2-carboxamide (5.1 mg, 0.0148 mmol, 3.4%) as a white powder. MS (ES+) m/z 345.3 (M+H); 1H-NMR (300 MHz, MeOD) δ 7.53 (d, J=2.3 Hz, 1H), 7.00-6.91 (m, 1H), 6.73 (d, J=8.2 Hz, 1H), 6.04 (d, J=2.3 Hz, 1H), 3.87 (s, 3H), 2.20 (s, 3H), 2.15 (s, 3H).

### 5-((1-(2.2-Difluoroethyl)-4-methyl-1H-pyrazol-3-yl)amino)-1,3,4-thiadiazole-2-carboxylic acid

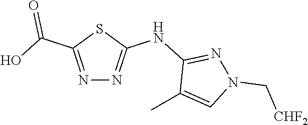 [SMILES: Cc1cn(CC(F)F)nc1Nc1nnc(C(=O)O)s1]

A mixture of ethyl 5-chloro-1,3,4-thiadiazole-2-carboxylate (50 mg, 0.260 mmol), 1-(2,2-difluoroethyl)-4-methyl-1H-pyrazol-3-amine (50 mg, 0.310 mmol) and 4-toluenesulfonic acid monohydrate (50 mg, 0.261 mmol) in IPA (0.6 mL) was heated at 80° C. for 18 h. Aq. NaHCO₃ (2 mL) was added, the reaction mixture was filtered, the filtrate was extracted with EtOAc (2×5 mL), the organic layers were washed with water (2×10 mL) then with brine (5 mL), combined, dried (Na₂SO₄) and evaporated. The crude material was purified by column chromatography over silica
(12 g cartridge) eluting with a gradient of EtOAc (30% to 80%; v/v) in isohexane to give the title compound (46 mg, 0.145 mmol, 55.85% yield) as an off-white solid. MS (ES+) m/z=332.1 [M+H]⁺; ¹H NMR (400 MHz, DMSO-d₆) δ 11.56 (s, 1H), 7.57 (s, 1H), 6.31 (tt, J=54.9, 3.7 Hz, 1H), 4.52 (td, J=15.1, 3.7 Hz, 2H), 4.37 (q, J=7.1 Hz, 2H), 2.03 (d, J=1.0 Hz, 3H), 1.37-1.29 (m, 3H). ¹⁹F NMR (376 MHz, DMSO-d₆) 0-122.52 (dt, J=54.9, 15.1 Hz).

A solution of ethyl 5-[1-(2,2-difluoroethyl)-4-methyl-pyrazol-3-yl]amino]-1,3,4-thiadiazole-2-carboxylate (54 mg, 0.170 mmol) in 1M NaOH aq. (0.25 mL, 0.250 mmol) and THF (0.8 mL) was stirred at 20° C. for 18 h. 1M HCl (0.4 mL) was added and the precipitate was collected, washed with water (1 mL), EtOAc then DCM, and dried (vacuum oven) to give 5-[1-(2,2-difluoroethyl)-4-methyl-pyrazol-3-yl]amino]-1,3,4-thiadiazole-2-carboxylic acid (24 mg, 0.0830 mmol, 48.75% yield) as an off-white solid. MS (ES+) m/z=290.2 [M+H]⁺, ¹H NMR (400 MHz, DMSO-d₆) δ 10.68 (s, 1H), 8.84 (s, 1H), 7.51 (s, 1H), 6.29 (tt, J=55.1, 3.9 Hz, 1H), 4.46 (td, J=14.9, 3.8 Hz, 2H), 1.99 (d, J=0.8 Hz, 3H).

### 5-((1.4-Dimethyl-1H-pyrazol-3-yl)amino)-1,3,4-thiadiazole-2-carboxylic acid

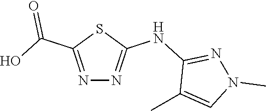 [SMILES: Cc1cn(C)nc1Nc1nnc(C(=O)O)s1]

A mixture of ethyl 5-chloro-1,3,4-thiadiazole-2-carboxylate (97 mg, 0.504 mmol), 1,4-dimethyl-1H-pyrazol-3-amine (69 mg, 0.621 mmol) and 4-toluenesulfonic acid monohydrate (93 mg, 0.489 mmol) in Ethanol (2.5 mL) was heated at 75° C. for 22h. Aq. NaHCO₃ (2 mL) and EtOAc
(2 mL) were added, the reaction mixture was filtered, the filtrate mixture was extracted with EtOAc
(2×5 mL). The organic layers were washed with brine (5 mL), combined, dried (Na₂SO₄) and evaporated. The crude material was purified by column chromatography over silica (4 g cartridge) eluting with a gradient of EtOAc (10% to 100%; v/v) in isohexane to give ethyl 5-[(1,4-dimethylpyrazol-3-yl)amino]-1,3,4-thiadiazole-2-carboxylate (16 mg, 12% yield) as an off-white solid. MS (ES+) m/z=268.2 [M+H]+. ¹H NMR (400 MHz, DMSO-d₆) δ 11.46 (s, 1H), 7.47 (s, 1H), 4.36 (q, J=7.1 Hz, 2H), 3.75 (s, 3H), 2.00 (d, J=0.8 Hz, 3H), 1.33 (t, J=7.1 Hz, 3H).

Ethyl 5-[(1,4-dimethylpyrazol-3-yl)amino]-1,3,4-thiadiazole-2-carboxylate (1.00 eq, 37 mg, 0.138 mmol) was partially dissolved in a mixture of THF (2.0 mL) and H₂O (1.0 mL) at r.t., NaOH
(2.71 eq, 15 mg, 0.375 mmol) was added, and the reaction mixture was stirred at r.t. for 18 h. and at 40° C. with additional NaOH, 1 M aq. (2.17 eq, 300 μL, 0.300 mmol for 1 h before being concentrated and dried in a vacuum oven at 40° C. to afford the title compound (sodium salt)(62 mg, 0.142 mmol, 102.88% yield) as a sticky yellow solid. ¹H NMR (400 MHz, MeOD) δ 7.19 (d, J=0.9 Hz, 1H), 3.73 (s, 3H), 1.99 (d, J=0.8 Hz, 3H).

### 5-((1-Cyclobutyl-1H-pyrazol-3-yl)amino)-1,3.4-thiadiazole-2-carboxylic acid

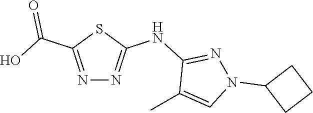 [SMILES: Cc1cn(C2CCC2)nc1Nc1nnc(C(=O)O)s1]

A mixture of ethyl 5-chloro-1,3,4-thiadiazole-2-carboxylate (98 mg, 0.509 mmol), 1-cyclobutylpyrazol-3-amine (83 mg, 0.608 mmol) and 4-toluenesulfonic acid monohydrate (97 mg, 0.510 mmol) in ethanol (2.5 mL) was heated at 75° C. for 18 h, aq. NaHCO₃ (2 mL) and EtOAc (2 mL) were added. A small amount of solid was collected and dried to give ethyl 5-[(1-cyclobutylpyrazol-3-yl)amino]-1,3,4-thiadiazole-2-carboxylate (35 mg, 23.5% yield) as a white solid. The filtrate mixture was extracted with EtOAc (2×5 mL). The organic layers were washed with brine (3 mL), combined, dried (Na₂SO₄) and evaporated. All the crude material (solid and mother liquors) was purified by column chromatography over silica (12 g cartridge) eluting with a gradient of EtOAc (30% to 90%; v/v) in isohexane to give ethyl 5-[(1-cyclobutylpyrazol-3-yl)amino]-1,3,4-thiadiazole-2-carboxylate (44 mg, 0.124 mmol, 24.47% yield) as an off-white solid. MS (ES+) m/z=294.2 [M+H]⁺; ¹H NMR (400 MHz, DMSO-d₆) δ 11.78 (s, 1H), 7.78 (dd, J=2.4, 1.0 Hz, 1H), 5.95 (d, J=2.3 Hz, 1H), 4.85-4.72 (m, 1H), 4.38 (q, J=7.1 Hz, 2H), 2.49-2.31 (m, 4H), 1.88-1.68 (m, 2H), 1.33 (t, J=7.1 Hz, 3H).

A solution of ethyl 5-[(1-cyclobutylpyrazol-3-yl)amino]-1,3,4-thiadiazole-2-carboxylate (76 mg, 0.259 mmol) in NaOH, 1M aq (0.50 mL, 0.500 mmol) and THF (1 mL) was stirred at rt for 40 h and at 40° C. for 6 h, with addition of additional NaOH 1M aq. The reaction mixture was concentrated under reduced pressure and dried in a vacuum oven overnight at 40° C. to afford sodium 5-[(1-cyclobutylpyrazol-3-yl)amino]-1,3,4-thiadiazole-2-carboxylate (112 mg, 0.253 mmol, 97.82% yield) as a bright yellow solid, which was used without further purification. MS (ES+) m/z=266.1 [M+H]⁺; ¹H NMR (400 MHz, MeOD) δ 8.55 (s, 1H), 7.49 (d, J=2.4 Hz, 1H), 5.93 (d, J=2.4 Hz, 1H), 4.71 (dtd, J=8.9, 8.0, 6.9 Hz, 1H), 2.70-2.57 (m, 2H), 2.45-2.35 (m, 2H), 1.91-1.85 (m, 2H).

### General Method Y¹: Amide Formation Between Methoxyaniline and 2-Pyrazoleamine Thiadiazole Carboxylic Acid and Methoxy Deprotection

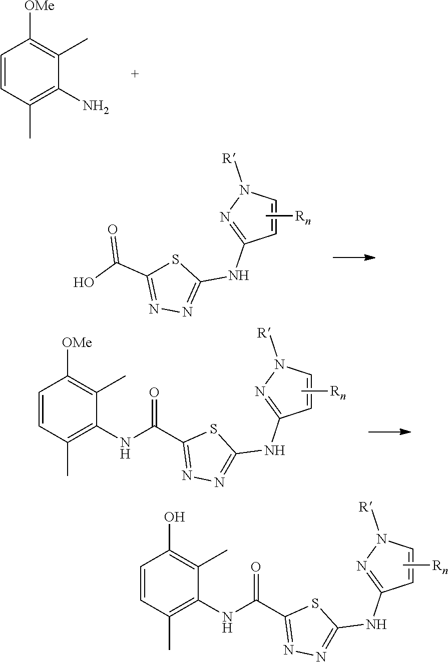

Thionyl chloride (15 eq) was added to a suspension of carboxylic acid (1 eq) and DMF (0.05 mL) in DCM. The reaction mixture was stirred at r.t. for 2.5 h before being concentrated under reduced pressure and azeotroped with toluene (5.0 mL). A mixture of methoxyaniline (1.2 eq) and caesium carbonate (2 eq) in 1,4-dioxane and MeCN under N₂ was heated at 90° C. for 5-10 minutes. The solution of acid chloride in dry 1,4-dioxane and MeCN was added dropwise at 90° C. and the reaction mixture was stirred for 2 h at 90° C. and for 16 h at rt. The reaction mixture was diluted with EtOAc and washed with water, brine, dried over sodium sulfate filtered and evaporated. The methoxy product was suspended in DCM, BBr₃ (9-12 eq) is added and the reaction mixture was stirred for 3 h, then quenched with MeOH, evaporated and the crude material was purified by reverse phase chromatography to afford the phenol product.

### 5-((1-(2,2-Difluoroethyl)-4-methyl-1H-pyrazol-3-yl)amino)-N-(3-hydroxy-2,6-dimethylphenyl)-1,3,4-thiadiazole-2-carboxamide( ) BAA-464

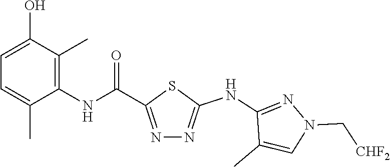 [SMILES: Cc1cn(CC(F)F)nc1Nc1nnc(C(=O)Nc2c(C)ccc(O)c2C)s1]

Prepared as described in method Y¹ from methoxyaniline (1.2 eq), caesium carbonate (2 eq) and 5-[1-(2,2-difluoroethyl)-4-methyl-pyrazol-3-yl]amino]-1,3,4-thiadiazole-2-carboxylic acid
(1.00 eq, 31 mg, 0.0864 mmol) to afford the methoxy intermediate 5-[1-(2,2-difluoroethyl)-4-methyl-pyrazol-3-yl]amino]-N-(3-methoxy-2,6-dimethyl-phenyl)-1,3,4-thiadiazole-2-carboxamide
(117 mg, 0.0831 mmol, 96.13% yield). MS (ES+) m/z=423.4, [M+H]⁺; ¹H NMR (400 MHz, CDCl₃) δ 9.85 (br s, 1H), 8.46 (s, 1H), 7.22 (d, J=1.0 Hz, 1H), 7.08 (d, J=8.4 Hz, 1H), 6.79 (d, J=8.4 Hz, 1H), 6.10 (dt, J=55.6, 4.2 Hz, 1H), 4.34 (td, J=13.3, 4.3 Hz, 2H), 3.83 (s, 3H), 2.25 (s, 2H), 2.18 (s, 3H), 2.17-2.16 (m, 3H). ¹⁹F NMR (376 MHz, CDCl₃) δ-122.69 (dt, J=55.7, 13.4 Hz). The intermediate was deprotected with BBr_{3 1} M in DCM (12.0 eq, 1.0 mL, 1.00 mmol) to afford to afford the title compound (14 mg, 0.0335 mmol, 40.36% yield) as a white solid after purification by prep HPLC. MS (ES+) m/z=409.3, [M+H]+ and 431.3 [M+Na]+; ¹H NMR (400 MHz, DMSO-d₆) δ 11.37 (s, 1H), 10.22 (s, 1H), 9.20 (s, 1H), 7.56 (d, J=1.0 Hz, 1H), 6.88 (d, J=8.2 Hz, 1H), 6.69 (d, J=8.2 Hz, 1H), 6.31 (tt, J=55.0, 3.8 Hz, 1H), 4.51 (td, J=15.0, 3.8 Hz, 2H), 2.07 (s, 3H), 2.03 (d, J=0.8 Hz, 3H), 1.98 (s, 3H). ¹⁹F NMR (376 MHz, DMSO-d₆) δ-122.53 (dt, J=55.0, 14.9 Hz).

The following example compounds were prepared similarly using Method Y¹ with appropriate carboxylic acids.

| # | Structure & Name | Analytical Data |
|---|---|---|
| BAA-465 | 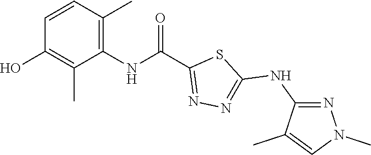 [SMILES: Cc1cn(C)nc1Nc1nnc(C(=O)Nc2c(C)ccc(O)c2C)s1] | ¹H NMR (400 MHz, DMSO-d₆) δ 11.27 (br s, 1H), 10.19 (s, 1H), 9.24 (s, 1H), 7.46 (s, 1H), 6.88 (d, J = 8.2 Hz, 1H), 6.69 (d, J = 8.2 Hz, 1H), 3.74 (s, 3H), 2.07 (s, 3H), 2.01 (s, 3H), 1.98 (s, 3H). MS (ES⁺) m/z 359.3 (M + H)⁺. |
| | 5-((1,4-dimethyl-1H-pyrazol-3- | |
| | yl)amino)-N-(3-hydroxy-2,6- | |
| | dimethylphenyl)-1,3,4-thiadiazole-2- | |
| | carboxamide | |
| BAA-466 | 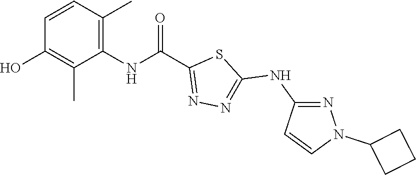 [SMILES: Cc1ccc(O)c(C)c1NC(=O)c1nnc(Nc2ccn(C3CCC3)n2)s1] | ¹H NMR (400 MHz, DMSO-d₆) δ 11.62 (s, 1H), 10.20 (s, 1H), 9.25 (s, 1H), 7.76 (d, J = 2.3 Hz, 1H), 6.88 (d, J = 8.2 Hz, 1H), 6.70 (d, J = 8.2 Hz, 1H), 5.93 (d, J = 2.4 Hz, 1H), 4.78 (p, J = 8.3 Hz, 1H), 2.48-2.42 (m, 2H), 2.39-2.30 (m, 2H), 2.07 (s, 3H), 1.99 (s, 3H), 1.88-1.67 (m, 2H). MS (ES⁺) m/z 385.3 (M + H)⁺. |
| | 5-((1-cyclobutyl-1H-pyrazol-3- | |
| | yl)amino)-N-(3-hydroxy-2,6- | |
| | dimethylphenyl)-1,3,4-thiadiazole-2- | |
| | carboxamide | |

Thionyl chloride (100 μL, 1.37 mmol) was added to a suspension of 5-[1-(2,2-difluoroethyl)-4-methyl-pyrazol-3-yl]amino]-1,3,4-thiadiazole-2-carboxylic acid (1.00 eq, 31 mg, 0.0864 mmol) and DMF (0.0500 mL) in DCM (1 mL). The reaction mixture was stirred at r.t. for 2.5 h before being concentrated under reduced pressure and azeotroped with toluene (5.0 mL). The residue was dissolved in 1,4-dioxane (1 mL) and MeCN (1 mL) and added to the warmed amine mixture below. In a separate flask, caesium carbonate (3.19 eq, 90 mg, 0.276 mmol) was added to 3-methoxy-2,6-dimethylaniline (1.15 eq, 15 mg, 0.0992 mmol) in 1,4-dioxane (1 mL) and MeCN (1 mL). The partial suspension was heated to 90° C. for 5 minutes before the addition of the acid chloride suspension.

The reaction mixture was heated at 90° C. for 2.5 h and stirred at r.t. overnight. The reaction mixture was diluted with EtOAc (25 mL) and H₂O (25 mL), the layers were separated, the organic layer was washed with brine (25 mL), dried over Na₂SO₄, filtered and the filtrates concentrated to afford 5-[1-(2,2-difluoroethyl)-4-methyl-pyrazol-3-yl]amino]-N-(3-methoxy-2,6-dimethyl-phenyl)-1,3,4-thiadiazole-2-carboxamide (117 mg, 0.0831 mmol, 96.13% yield) as a yellow solid/oily residue, which was used without further purification. MS (ES+) m/z=423.4, [M+H]⁺; ¹H NMR (400 MHz, CDCl₃) δ 9.85 (br s, 1H), 8.46 (s, 1H), 7.22 (d, J=1.0 Hz, 1H), 7.08 (d, J=8.4 Hz, 1H), 6.79 (d, J=8.4 Hz, 1H), 6.10 (dt, J=55.6, 4.2 Hz, 1H), 4.34 (td, J=13.3, 4.3 Hz, 2H), 3.83 (s, 3H), 2.25 (s, 2H), 2.18 (s, 3H), 2.17-2.16 (m, 3H). ¹⁹F NMR (376 MHz, CDCl₃) δ-122.69 (dt, J=55.7, 13.4 Hz).

5-[1-(2,2-difluoroethyl)-4-methyl-pyrazol-3-yl]amino]-N-(3-methoxy-2,6-dimethyl-phenyl)-1,3,4-thiadiazole-2-carboxamide (1.00 eq, 117 mg, 0.0831 mmol) was dissolved in DCM (2.0 mL) at r.t., boron tribromide, 1 M in DCM (12.0 eq, 1.0 mL, 1.00 mmol) was added, and the reaction mixture was stirred at r.t. for 18 h, then it was quenched with MeOH(10 mL) and concentrated under reduced pressure. The crude residue was purified by prep HPLC to afford the title compound
(14 mg, 0.0335 mmol, 40.36% yield) as a white solid.MS (ES+) m/z=409.3, [M+H]+ and 431.3 [M+Na]+; ¹H NMR (400 MHz, DMSO-d₆) δ 11.37 (s, 1H), 10.22 (s, 1H), 9.20 (s, 1H), 7.56 (d, J=1.0 Hz, 1H), 6.88 (d, J=8.2 Hz, 1H), 6.69 (d, J=8.2 Hz, 1H), 6.31 (tt, J=55.0, 3.8 Hz, 1H), 4.51 (td, J=15.0, 3.8 Hz, 2H), 2.07 (s, 3H), 2.03 (d, J=0.8 Hz, 3H), 1.98 (s, 3H). ¹⁹F NMR (376 MHz, DMSO-d₆) δ-122.53 (dt, J=55.0, 14.9 Hz).

### Synthesis of Example BAA-220

### N-(3-hydroxy-2,6-dimethyl-phenyl)-2-[[6-[4-(2-hydroxyethyl) piperazin-1-yl]-2-methyl-pyrimidin-4-yl]amino]thiazole-5-carboxamide (BAA-220)

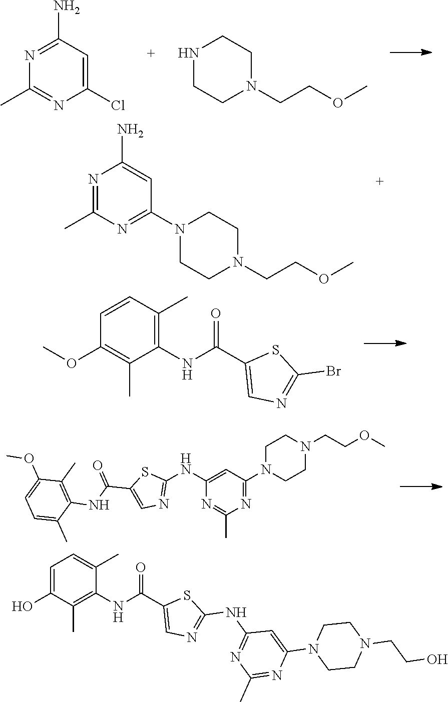

Step 1: To a solution of 4-amino-6-chloro-2-methylpyrimidine (400 mg, 2.79 mmol, 1.0 eq) in MeCN (3.2 mL) and DMF (0.8 mL) were added 1-(2-methoxyethyl) piperazine (0.46 mL, 3.06 mmol, 1.1 eq) and K₂CO₃ (0.57 mL, 3.06 mmol, 1.1 eq). The reaction mixture was irradiated under MW (200W) in a sealed tube for 1.5 h at 120° C. After cooling to RT, the mixture was diluted with water (16 mL) and extracted with EtOAc (3×20 mL). The combined organic layers were washed with brine (20 mL), dried (MgSO₄), filtered and evaporated to dryness. The resulting residue was purified by reverse phase chromatography (C18) eluting with 5-95% MeCN: H₂O to afford 6-[4-(2-methoxyethyl) piperazin-1-yl]-2-methyl-pyrimidin-4-amine (395 mg, 1.57 mmol, 56%) as a yellow powder. MS (ES+) m/z 252.0 (M+H). ¹H NMR (300 MHz, DMSO-d₆) δ 6.11 (s, 2H), 5.42 (s, 1H), 3.45 (t, J=5.8 Hz, 2H), 3.41-3.34 (m, 2H), 3.24 (s, 3H), 2.52-2.40 (m, 8H), 2.15 (s, 3H) ppm.

Step 2: Prepared using Method I from 2-bromo-N-(3-methoxy-2,6-dimethyl-phenyl)thiazole-5-carboxamide (265 mg, 0.777 mmol, 1.0 eq), 6-[4-(2-methoxyethyl) piperazin-1-yl]-2-methyl-pyrimidin-4-amine (390 mg, 1.55 mmol, 2.0 eq), Pd₂dba₃ (107 mg, 0.116 mmol, 0.15 eq), XantPhos
(135 mg, 0.233 mmol, 0.3 eq) and Cs₂CO₃ (764 mg, 2.33 mmol, 3.00 eq) in 1,4-dioxane (14 mL) and water (1.6 mL) to afford N-(3-methoxy-2,6-dimethyl-phenyl)-2-[6-[4-(2-methoxyethyl) piperazin-1-yl]-2-methyl-pyrimidin-4-yl]amino]thiazole-5-carboxamide (51.5 mg, 0.079 mmol, 10%) after normal phase chromatography (SiO₂) eluting with 0-20% MeOH: DCM. The residue was used directly in the next step without further purification. MS (ES+) 512.3 (M+H).

Step 3: Prepared using Method K from N-(3-methoxy-2,6-dimethyl-phenyl)-2-[6-[4-(2-methoxyethyl) piperazin-1-yl]-2-methyl-pyrimidin-4-yl]amino]thiazole-5-carboxamide (51 mg, 0.0997 mmol, 1.0 eq) and BBr₃ (1M in DCM, 0.25 mL, 0.249 mmol, 2.5 eq) at RT for 2 h. Additional BBr₃
(0.1 mL, 0.0997 mmol, 1.0 eq) added and stirred for a further 1 h. The mixture was quenched with H₂O (1 mL), stirred at RT for 2 h then evaporated to dryness and purified by preparative HPLC-MS to afford the title compound (4.4 mg, 0.00910 mmol, 9.1%) as a white powder. MS (ES+) 484.3 (M+H). ¹H NMR (300 MHz, Methanol-d4) δ 8.46 (s, 1H), 8.14 (s, 1H), 6.94 (d, J=8.2 Hz, 1H), 6.71 (d, J=8.2 Hz, 1H), 6.05 (s, 1H), 3.85-3.70 (m, 6H), 2.92-2.77 (m, 6H), 2.50 (s, 3H), 2.18 (s, 3H), 2.14 (s, 3H).

### Phenol Substituents Modifications

### Phenol Building Blocks

### 3-[tert-Butyl(dimethyl) silyl]oxy-6-chloro-2-methyl-aniline

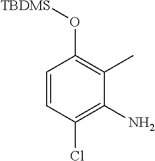

Tert-Butyldimethylchlorosilane (3122 mg, 20.7 mmol) was added into the solution of imidazole (2350 mg, 34.5 mmol) and 3-amino-4-chloro-2-methyl-phenol (1360 mg, 8.63 mmol) in dry THF (80 mL) under N₂ at rt for 18 h. The reaction mixture was diluted with water (100 mL) and EtOAc (100 mL). The organic layer was washed with water (2×100 mL), dried with Na₂SO₄, filtered off and concentrated in vacuo to provide 3-[tert-butyl(dimethyl) silyl]oxy-6-chloro-2-methyl-aniline (2607 mg, 7.67 mmol, 88.90% yield) as a brown oil. MS (ES+) m/z=272.1/274.1 [M+H]+. ¹H NMR (400 MHz, DMSO-D6) δ 6.93 (dd, J=8.7, 0.6 Hz, 1H), 6.14-6.09 (m, 1H), 4.98 (s, 2H), 1.98 (d, J=0.5 Hz, 3H), 0.97 (s, 9H), 0.16 (s, 6H).

### 2-Bromo-3-[tert-butyl(dimethyl) silyl]oxy-6-methyl-aniline

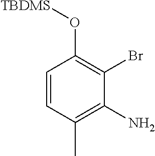

Tetrahydroxy diboron (2.57 eq, 0.84 g, 9.37 mmol) was added portion wise to a stirred solution of 2-bromo-4-methyl-3-nitro-phenol (1.00 eq, 0.85 g, 3.64 mmol) and 4,4′-dipyridyl (0.0334 eq, 19 mg, 0.122 mmol) in DMF (10 mL). After 2h, further tetrahydroxy diboron (0.93 g) was added and the stirring continued for 1 h. The reaction was quenched with sat. aq. NH₄Cl (50 mL) and extracted with EtOAc (100 mL). The organic phase was washed with NH₄Cl (50 mL), brine, dried filtered and evaporated. The crude material was purified by column chromatography over C18 (140 g cartridge) eluting with a gradient of MeCN (0.1% NH₃)(5% to 95%; v/v) in water (0.1% NH₃) to afford 3-amino-2-bromo-4-methyl-phenol (430 mg, 1.70 mmol, 46.7%) as a brown solid. MS (ES-) m/z=200/202 [M-H]− mono Br pattern, ¹H NMR (400 MHz, DMSO-D6) δ 9.57 (s, 1H), 6.71 (dd, J=8.0, 0.9 Hz, 1H), 6.12 (d, J=8.0 Hz, 1H), 4.84 (s, 2H), 2.03 (d, J=0.7 Hz, 3H).

tert-Butyldimethylchlorosilane (2.43 eq, 760 mg, 5.04 mmol) was added to a mixture of 3-amino-2-bromo-4-methyl-phenol (1.00 eq, 420 mg, 2.08 mmol) and imidazole (4.03 eq, 570 mg, 8.37 mmol) in THF (20 mL). After 2 h the reaction mixture was diluted with EtOAc, washed with water (x2) and evaporated. The crude material was purified by column chromatography over silica
(12 g cartridge) eluting with a gradient of EtOAc (0% to 30%; v/v) in isohexane [note 3], to afford the title product (430 mg, 1.35 mmol, 64.74% yield) as a colourless oil. MS (ES+) m/z=316.0/317.9 [M+H]+. ¹H NMR (400 MHz, CHLOROFORM-D) δ 6.83 (dt, J=8.1, 0.8 Hz, 1H), 6.25 (d, J=8.1 Hz, 1H), 4.10 (s, 2H), 2.15 (d, J=0.7 Hz, 3H), 1.04 (d, J=0.6 Hz, 9H), 0.23 (d, J=0.6 Hz, 6H).

### 6-Bromo-3-[tert-butyl(dimethyl) silyl]oxy-2-methyl-aniline

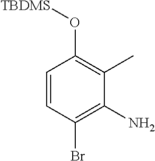

Imidazole (1159 mg, 17.0 mmol) and tert-butyldimethylchlorosilane (1540 mg, 10.2 mmol) were added sequentially into the solution of 3-amino-4-bromo-2-methyl-phenol (1000 mg, 4.26 mmol) in dry THF (39.4 mL) at rt under N₂ and stirred for 18 h. The reaction mixture was quenched with the addition of water (100 mL) and EtOAc (100 mL). The water layer was extracted with EtOAc (3×75 mL) and the combined organic layer was dried with Na₂SO₄, filtered off and dried in vacuo to provide the title compound (1650 mg, 4.23 mmol, 99.27% yield) as a brown oil. MS (ES+) m/z=316.0/317.9 [M+H]+. ¹H NMR (400 MHz, DMSO-D6) δ 7.08 (dd, J=8.7, 0.6 Hz, 1H), 6.09 (d, J=8.7 Hz, 1H), 4.94 (s, 2H), 2.00 (s, 3H), 0.97 (s, 9H), 0.16 (s, 6H).

### 2-Ethyl-3-methoxy-6-methylaniline

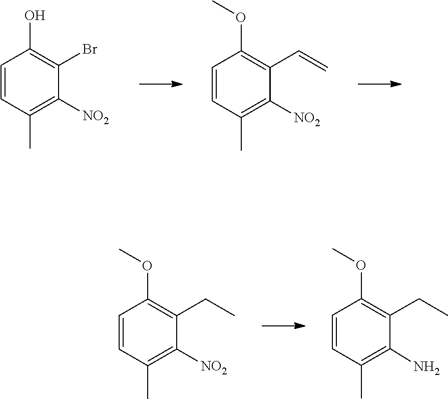

Potassium carbonate 325 mesh (2.10 eq, 2.00 g, 14.5 mmol) was added to a solution of 2-bromo-4-methyl-3-nitro-phenol (1.00 eq, 1.60 g, 6.90 mmol) in MeCN (15 mL) and water (5 mL), followed by potassium vinyltrifluoroborate (1.08 eq, 1.00 g, 7.47 mmol) and 1,1′-bis(diphenylphosphino) ferrocenepalladium (II) dichloride (0.0565 eq, 285 mg, 0.390 mmol). The reaction mixture was heated at 80° C. for 24 h, cool to rt and iodomethane (3.03 eq, 1.3 mL, 20.9 mmol) added. After stirring at rt for 48 h further iodomethane (1.0 mL) and potassium carbonate 325 mesh (1.00 g) added, and the stirring continued for for 3 days. The reaction mixture was diluted with water and EtOAc and filtered. The phases were separated and the organic phase washed with NaHCO₃aq, brine, dried over sodium sulfate, filtered, evaporated and purified by column chromatography over silica (80 g cartridge) eluting with a gradient of EtOAc (0% to 50%; v/v) in isohexane to afford 1-methoxy-4-methyl-3-nitro-2-vinyl-benzene (340 mg, 1.76 mmol, 25.52% yield). ¹H NMR (400 MHz, CHLOROFORM-D) § 7.16-7.09 (m, 1H), 6.89 (d, J=8.5 Hz, 1H), 6.57 (dd, J=17.8, 11.8 Hz, 1H), 5.74 (dd, J=17.9, 1.4 Hz, 1H), 5.49 (dd, J=11.8, 1.4 Hz, 1H), 3.86 (s, 3H), 2.22 (d, J=0.7 Hz, 3H).

A slurry of palladium on activated carbon (0.340 eq, 60 mg, 0.564 mmol) in MeCN (2 mL) was added under nitrogen to a solution of 1-methoxy-4-methyl-3-nitro-2-vinyl-benzene (1.00 eq, 320 mg, 1.66 mmol) in MeCN (20 mL). The reaction mixture was stirred under a hydrogen atmosphere for 24 h, the catalyst was filtered off through celite, washed through with MeCN and the filtrate evaporated to afford 2-ethyl-1-methoxy-4-methyl-3-nitro-benzene (300 mg, 1.54 mmol, 92.78% yield) which was used in the next step without further purification.

Tetrahydroxy diboron (5.88 eq, 0.81 g, 9.04 mmol) was added portion-wise to a solution of 2-ethyl-1-methoxy-4-methyl-3-nitro-benzene (1.00 eq, 300 mg, 1.54 mmol) and 4,4′-dipyridyl
(0.125 eq, 30 mg, 0.192 mmol) in DMF 7 mL) with external cold water bath, under nitrogen. After 1 h the reaction mixture was quenched with NaHCO₃aq and extracted into EtOAc (×2). The combined organic phases were washed with brine (x2), then dried over sodium sulfate, filtered and evaporated. The crude material was purified by column chromatography over silica (24 g cartridge) eluting with a gradient of EtOAc (0% to 95%; v/v) in isohexane to afford 2-ethyl-3-methoxy-6-methyl-aniline (205 mg, 1.24 mmol, 80.73% yield) as colourless oil. MS (ES+) m/z=166 [M+H]+, ¹H NMR (400 MHz, CHLOROFORM-D) δ 6.87 (dq, J=8.3, 0.8 Hz, 1H), 6.30 (d, J=8.2 Hz, 1H), 3.78 (s, 3H), 3.63 (s, 2H), 2.60 (q, J=7.6 Hz, 2H), 2.13 (d, J=0.8 Hz, 3H), 1.12 (t, J=7.6 Hz, 3H).

### N-(6-Bromo-3-hydroxy-2-methyl-phenyl)-2-chloro-thiazole-5-carboxamide

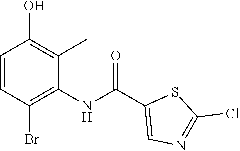 [SMILES: Cc1c(O)ccc(Br)c1NC(=O)c1cnc(Cl)s1]

Thionyl chloride (0.25 mL, 3.43 mmol) was added to a suspension of 2-bromothiazole-5-carboxylic acid (1.00 eq, 180 mg, 0.865 mmol) and DMF (0.05 mL) in DCM (10 mL). The reaction mixture was stirred at ambient temperature for 2.5 hours, evaporated, redissolved in 1,4-dioxane (3 mL) and MeCN (3 mL) and added to the warmed amine mixture below.

In a separate flask caesium carbonate (3.19 eq, 900 mg, 2.76 mmol) was added to 6-bromo-3-[tert-butyl(dimethyl) silyl]oxy-2-methyl-aniline (0.932 eq, 315 mg, 0.807 mmol) in 1,4-dioxane (3 mL) and MeCN (3 mL) and heated to 90° C. for 5 minutes before addition of acid chloride suspension above. The reaction mixture was stirred at 90° C. for 2.5 h and at ambient temp overnight. Hydrogen chloride (6.93 eq, 0.50 mL, 6.00 mmol) [cHCl] was added and the mixture stirred at ambient for 24 h. The reaction mixture was dilute with MeCN, the solids were filtered off, the filtrate evaporated and the crude material was purified by column chromatography over C18 (23 g cartridge) eluting with a gradient of MeCN (0.1% formic acid)(5% to 95%; v/v) in water (0.1% formic acid) to afford the N-(6-bromo-3-hydroxy-2-methyl-phenyl)-2-chloro-thiazole-5-carboxamide (145 mg, 0.388 mmol, 44.83% yield) as a white solid. MS (ES+) m/z=344.9/346.9/348.9 [M-H]− mono Cl mono Br pattern. ¹H NMR (400 MHz, DMSO-D6) δ 10.37 (s, 1H), 9.87 (s, 1H), 8.45 (s, 1H), 7.34 (dd, J=8.8, 0.7 Hz, 1H), 6.79 (d, J=8.7 Hz, 1H), 2.03 (s, 3H).

### 2-Bromo-N-(2-bromo-3-((tert-butyldimethylsilyl)oxy)-6-methylphenyl)thiazole-5-carboxamide

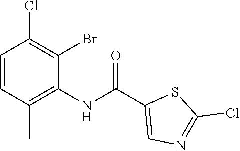 [SMILES: Cc1ccc(Cl)c(Br)c1NC(=O)c1cnc(Cl)s1]

Thionyl chloride (0.38 mL, 5.19 mmol) was added to a suspension of 2-bromothiazole-5-carboxylic acid (1.00 eq, 270 mg, 1.30 mmol) and DMF (0.1000 mL) in DCM (3 mL) and the suspension was stirred at ambient temperature for 2 hours then evaporated. The residue was dissolved in 1,4-Dioxane (1 mL) and MeCN (1 mL) and added to the warmed amine mixture below. In a separate flask caesium carbonate (3.00 eq, 1269 mg, 3.89 mmol) was added to 2-bromo-3-[tert-butyl(dimethyl) silyl]oxy-6-methyl-aniline (411 mg, 1.30 mmol) in 1,4-dioxane (2 mL) and MeCN
(2 mL) and heated at 90° C. for 5 minutes. The above solution of acid chloride was added and the mixture was heated at 90° C. for 2.5 h then stirred at ambient temperature overnight. Aq. 1M HCl (5 mL) then aq. NaHCO₃ (2 mL) were added and mixture was extracted with EtOAc (3×10 mL). The organic layers were washed with brine (5 mL), combined, dried (Na₂SO₄) and evaporated to give a brown oil. This residue was dissolved in MeOH(5 mL), conc. HCl (0.10 mL, 1.20 mmol) was added and the mixture was stood for 3 days. Aq. NaHCO₃ (2 mL) and brine (2 mL) were added and mixture was extracted with EtOAc (4×10 mL). The organic layers were washed with brine (5 mL), combined, dried (Na₂SO₄), evaporated and the crude material was purified by column chromatography over silica (40 g cartridge) eluting with a gradient of EtOAc (20% to 100%; v/v) in isohexane to afford (2-bromo-3-hydroxy-6-methyl-phenyl)-2-chloro-thiazole-5-carboxamide (227 mg, 0.366 mmol, 28.27% yield) as an orange gum. MS (ES+) m/z=344.9/346.9/348.9 [M-H]−, Br/Cl splitting pattern.

### 2-Bromo-N-(3-methoxy-2,4,6-trimethylphenyl)thiazole-5-carboxamide

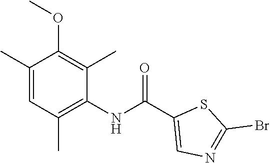 [SMILES: COc1c(C)cc(C)c(NC(=O)c2cnc(Br)s2)c1C]

DIPEA (1.0 mL, 5.81 mmol) and 1-Propanephosphonic anhydride (2.8 mL, 4.84 mmol) were added into the mixture of 3-methoxy-2,4,6-trimethyl-aniline (400 mg, 1.94 mmol) and 2-bromothiazole-5-carboxylic acid (604 mg, 2.90 mmol) in dry MeCN (19.4 mL) at rt under N₂ and the reaction mixture was stirred for 18 h, then diluted with water (50 mL) and EtOAc (50 mL). The water layer was washed with EtOAc (3×50 mL). The combined organic layer was washed with brine (30 mL), dried with Na₂SO₄, filtered off and concentrated in vacuo to provide 2-bromo-N-(3-methoxy-2,4,6-trimethyl-phenyl)thiazole-5-carboxamide (400 mg, 0.991 mmol, 51.17% yield) as a brown solid. MS (ES+) m/z=355.0/357.0 [M+H]+, ¹H NMR (400 MHz, DMSO-D6) δ 10.07 (s, 1H), 8.42 (s, 1H), 6.97 (s, 1H), 3.62 (s, 3H), 2.21 (s, 3H), 2.09 (s, 3H), 2.07 (s, 3H).

### 2-Bromo-N-(4-chloro-3-methoxy-2,6-dimethyl-phenyl)thiazole-5-carboxamide

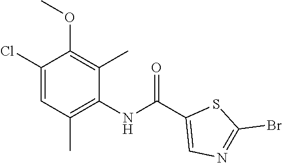 [SMILES: COc1c(Cl)cc(C)c(NC(=O)c2cnc(Br)s2)c1C]

N-Chlorosuccinimide (1.13 eq, 185 mg, 1.39 mmol) was added to a solution of 2-bromo-N-(3-methoxy-2,6-dimethyl-phenyl)thiazole-5-carboxamide (1.00 eq, 420 mg, 1.23 mmol) in MeCN
(10 mL), the reaction mixture was heated to 80° C. for 2.5h, cool and evaporated. The crude material was purified by column chromatography over C18 (90 g cartridge) eluting with a gradient of MeCN (0.1% NH₃)(5% to 95%; v/v) in water (0.1% NH₃) [note 3] to afford the desired product 2-bromo-N-(4-chloro-3-methoxy-2,6-dimethyl-phenyl)thiazole-5-carboxamide (230 mg, 0.588 mmol, 47.75% yield) as a beige solid.MS (ES-) m/z=373.0/375.0/377.0 [M-H]−. ¹H NMR (400 MHz, DMSO-D6) δ 10.18 (d, J=8.0 Hz, 1H), 8.40 (d, J=5.7 Hz, 1H), 7.27 (q, J=0.9 Hz, 1H), 3.69 (s, 3H), 2.10 (dd, J=2.6, 1.6 Hz, 6H).

### 2-Bromo-N-(2-chloro-6-fluoro-3-methoxyphenyl)thiazole-5-carboxamide

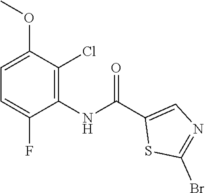 [SMILES: COc1ccc(F)c(NC(=O)c2cnc(Br)s2)c1Cl]

1-Propanephosphonic anhydride [50% EtOAc](1.50 eq, 1.0 mL, 1.71 mmol) and DIPEA
(4.03 eq, 0.80 mL, 4.59 mmol) were added to a solution of 2-bromothiazole-5-carboxylic acid (1.01 eq, 240 mg, 1.15 mmol) and 2-chloro-6-fluoro-3-methoxyaniline (1.00 eq, 200 mg, 1.14 mmol) in THF (10 mL) which was heated at 65° C. for 24 h. The reaction mixture was then diluted with EtOAc, washed with water, NaHCO₃aq, then brine, dried over sodium sulfate, filtered and evaporated. The crude material was purified by column chromatography over silica (12 g cartridge) eluting with a gradient of EtOAc (5% to 80%; v/v) in heptane to afford the title compound (140 mg, 0.314 mmol, 27.57% yield) as an orange gum. MS (ES-) m/z=363.0/365.0/367.0 [M-H]− mono Br mono Cl. ¹H NMR (400 MHz, CHLOROFORM-D) 8 8.08 (s, 1H), 7.94 (s, 1H), 7.10 (t, J=9.1 Hz, 1H), 6.89 (dd, J=9.3, 4.4 Hz, 1H), 3.91 (s, 3H). ¹⁹F NMR (376 MHz, CHLOROFORM-D) δ-124.25,-126.86 (dd, J=8.4, 4.7 Hz).

### 2-Bromo-N-(6-chloro-2-fluoro-3-methoxy-phenyl)thiazole-5-carboxamide

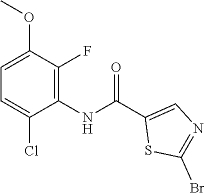 [SMILES: COc1ccc(Cl)c(NC(=O)c2cnc(Br)s2)c1F]

1-Propanephosphonic anhydride [50% EtOAc](1.50 eq, 1.0 mL, 1.71 mmol) and DIPEA
(4.03 eq, 0.80 mL, 4.59 mmol) were added to a solution of 2-bromothiazole-5-carboxylic acid (1.14 eq, 270 mg, 1.30 mmol) and 6-chloro-2-fluoro-3-methoxyaniline (1.00 eq, 200 mg, 1.14 mmol) in THF (10 mL), which was heated at 65° C. for 24 h. The reaction mixture was then diluted with EtOAc, washed with water, NaHCO₃aq, then brine, dried over sodium sulfate, filtered and evaporated. The crude material was purified by column chromatography over silica (12 g cartridge) eluting with a gradient of EtOAc (5% to 80%; v/v) in heptane to afford the title compound (170 mg, 0.405 mmol, 35.52% yield) as an orange gum. MS (ES+) m/z=363.0/365.0/367.0 [M-H]− mono Br mono CI pattern. ¹H NMR (400 MHz, CHLOROFORM-D) § 8.06 (s, 1H), 7.93 (s, 1H), 7.19 (dd, J=9.0, 2.2 Hz, 1H), 6.90 (t, J=8.7 Hz, 1H), 3.89 (s, 3H). ¹⁹F NMR (376 MHz, CHLOROFORM-D) 5-134.38 (d, J=8.4 Hz), -135.98 (dd, J=8.4, 2.1 Hz).

### 2-Bromo-N-(2-bromo-3-methoxy-6-methylphenyl)thiazole-5-carboxamide

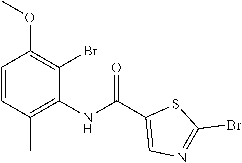 [SMILES: COc1ccc(C)c(NC(=O)c2cnc(Br)s2)c1Br]

To a solution of 2-bromo-3-methoxy-6-methyl-aniline (400 mg, 1.85 mmol), 2-bromothiazole-5-carboxylic acid (385 mg, 1.85 mmol) and DIPEA (0.97 mL, 5.55 mmol) in THF
(18.5 mL) was added 1-propanephosphonic anhydride (0.80 mL, 2.78 mmol) then the reaction was heated to 50° C. and stirred for 4 days. The reaction mixture was cooled to room temperature, concentrated under reduced pressure, the residue was precipitated in water, filtered and washed successively with water and hexane to afford the title compound (81 mg, 0.199 mmol, 10.78% yield) as a beige solid.MS (ES-) m/z=403.0/405.0/407.0 [M-H]−. ¹H NMR (400 MHz, DMSO-D6) o 10.39 (s, 1H), 8.45 (s, 1H), 7.29 (dd, J=8.5, 0.8 Hz, 1H), 7.04 (d, J=8.5 Hz, 1H), 3.85 (s, 3H), 2.16 (s, 3H).

### 2-Bromo-N-(6-chloro-2-fluoro-3-hydroxy-phenyl)thiazole-5-carboxamide

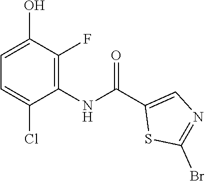 [SMILES: O=C(Nc1c(Cl)ccc(O)c1F)c1cnc(Br)s1]

Prepared as described in Method K from 2-bromo-N-(6-chloro-2-fluoro-3-methoxy-phenyl)thiazole-5-carboxamide (1.00 eq, 170 mg, 0.405 mmol) and BBr_{3 1}M DCM (4.94 eq, 2.0 mL, 2.00 mmol) in DCM (10 mL) to afford the title compound (80 mg, 0.180 mmol, 44.44% yield) as a white solid. MS (ES-) m/z=348.9/350.9/352.9 [M-H]-mono CI mono Br pattern. ¹H NMR (400 MHz, DMSO-D6) δ 10.52 (s, 1H), 10.37 (s, 1H), 8.45 (s, 1H), 7.22 (dd, J=8.9, 1.9 Hz, 1H), 6.98 (t, J=8.9 Hz, 1H).

### N-(6-Chloro-2-fluoro-3-hydroxy-phenyl)-2-[[1-(2-oxo-2-pyrrolidin-1-yl-ethyl)pyrazol-3-yl]amino]thiazole-5-carboxamide( ) BAA-481

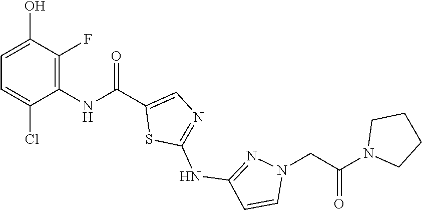 [SMILES: O=C(Nc1c(Cl)ccc(O)c1F)c1cnc(Nc2ccn(CC(=O)N3CCCC3)n2)s1]

Prepared as described in Method M from 2-(3-aminopyrazol-1-yl)-1-pyrrolidin-1-yl-ethanone (2.29 eq, 80 mg, 0.412 mmol), 2-bromo-N-(6-chloro-2-fluoro-3-hydroxy-phenyl)thiazole-5-carboxamide (1.00 eq, 80 mg, 0.180 mmol), potassium carbonate 325 mesh (3.22 eq, 80 mg, 0.579 mmol), Xantphos)(0.240 eq, 25 mg, 0.0432 mmol) and Palladium (II)acetate (0.100 eq, 4.0 mg, 0.0180 mmol) in 1,4-Dioxane (10 mL) to afford N-(6-chloro-2-fluoro-3-hydroxy-phenyl)-2-[1-(2-oxo-2-pyrrolidin-1-yl-ethyl)pyrazol-3-yl]amino]thiazole-5-carboxamide (15 mg, 0.0323 mmol, 17.95% yield) as a white solid after purification by column chromatography over C18 (23 g cartridge) eluting with a gradient of MeCN (0.1% formic acid)(5% to 95%; v/v) in water (0.1% formic acid). ¹H NMR (400 MHz, DMSO-d₆) δ 10.93 (s, 1H), 10.03 (s, 1H), 9.71 (s, 1H), 8.10 (s, 1H), 7.60 (d, J=2.3 Hz, 1H), 7.16 (dd, J=8.9, 1.9 Hz, 1H), 6.94 (t, J=8.9 Hz, 1H), 6.03 (d, J=2.3 Hz, 1H), 4.91 (s, 2H), 3.54 (t, J=6.8 Hz, 2H), 3.34 (t, J=6.9 Hz, 2H), 1.93 (p, J=6.8 Hz, 2H), 1.80 (p, J=6.8 Hz, 2H). MS (ES+) m/z 465.2/467.2 (M+H)⁺.

The following example compounds were prepared similarly using Method K from appropriately substituted 2-bromo-N-(3-methoxy-phenyl)thiazole-5-carboxamide followed by Method M with appropriate amines.

| # | Structure & Name | Analytical Data |
|---|---|---|
| BAA-482 | 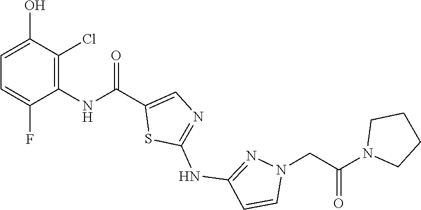 [SMILES: O=C(Nc1c(F)ccc(O)c1Cl)c1cnc(Nc2ccn(CC(=O)N3CCCC3)n2)s1] | ¹H NMR (400 MHz, DMSO-d₆) δ 10.91 (s, 1H), 9.98 (s, 1H), 9.69 (s, 1H), 8.10 (s, 1H), 7.60 (d, J = 2.4 Hz, 1H), 7.09 (t, J = 9.2 Hz, 1H), 6.93 (dd, J = 9.1, 4.9 Hz, 1H), 6.03 (d, J = 2.3 Hz, 1H), 4.91 (s, 2H), 3.54 (t, J = 6.8 Hz, 2H), 3.34 (t, J = 6.9 Hz, 2H), 1.93 (p, J = 6.8 Hz, 2H), 1.80 (p, J = 6.8 Hz, 2H). MS (ES⁺) m/z 465.2/467.2 (M + H)⁺. |
| | N-(2-chloro-6-fluoro-3-hydroxyphenyl)-2-((1-(2-oxo-2-(pyrrolidin- | |
| | 1-yl)ethyl)-1H-pyrazol-3-yl)amino) thiazole-5-carboxamide | |

### N-(6-Bromo-3-hydroxy-2-methyl-phenyl)-2-[[1-(2-oxo-2-pyrrolidin-1-yl-ethyl)pyrazol-3-yl]amino]thiazole-5-carboxamide( ) BAA-519

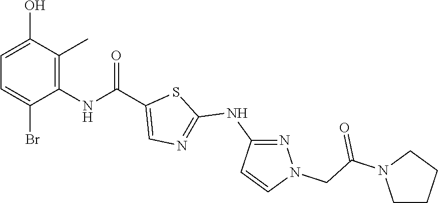 [SMILES: Cc1c(O)ccc(Br)c1NC(=O)c1cnc(Nc2ccn(CC(=O)N3CCCC3)n2)s1]

Prepared as described in method M from 2-(3-aminopyrazol-1-yl)-1-pyrrolidin-1-yl-ethanone (2.47 eq, 200 mg, 1.03 mmol), N-(6-bromo-3-hydroxy-2-methyl-phenyl)-2-chloro-thiazole-5-carboxamide (1 eq, 145 mg, 0.417 mmol), Xantphos (0.311 eq, 75 mg, 0.130 mmol), and Palladium (II)acetate (0.214 eq, 20 mg, 0.0891 mmol) and potassium carbonate 325 mesh
(4.34 eq, 250 mg, 1.81 mmol) in 1,4-dioxane (10 mL) to afford the title compound (2.4 mg, 0.00427 mmol, 1% yield) after purification by column chromatography over C18 (23 g cartridge), gradient of MeCN (0.1% formic acid)(5% to 95%; v/v) in water (0.1% formic acid), and preparative HPLC. MS (ES+) m/z=505.2/507.2 [M+H]+, mono Br pattern. ¹H NMR (400 MHz, DMSO-D6) δ 9.76 (s, 1H), 8.45 (s, 2H), 8.12 (s, 1H), 7.60 (d, J=2.3 Hz, 1H), 7.30 (d, J=8.7 Hz, 1H), 6.76 (d, J=8.7 Hz, 1H), 5.98 (d, J=2.3 Hz, 1H), 4.93 (s, 2H), 2.02 (s, 3H), 1.90 (q, J=6.7 Hz, 4H), 1.78 (p, J=6.6 Hz, 4H).

### N-(2-Bromo-3-hydroxy-6-methylphenyl)-2-((1-(cyanomethyl)-4-methyl-1H-pyrazol-3-yl)amino)thiazole-5-carboxamide( ) BAA-584

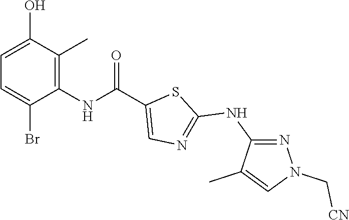 [SMILES: Cc1cn(CC#N)nc1Nc1ncc(C(=O)Nc2c(Br)ccc(O)c2C)s1]

Prepared as described in method M from potassium carbonate 325 mesh (74 mg, 0.539 mmol), palladium (II)acetate (4.1 mg, 0.0180 mmol), Xantphos (21 mg, 0.0361 mmol), 2-(3-amino-4-methyl-pyrazol-1-yl)acetonitrile (96 mg, 0.353 mmol) and 2-(3-amino-4-methyl-pyrazol-1-yl)acetonitrile (96 mg, 0.353 mmol) in 1,4-Dioxane (1 mL) to afford the title compound (17 mg, 0.0365 mmol, 20.25% yield) as a white lyophilised solid after purification by prep HPLC. MS (ES+) m/z=447.2/449.2 [M+H]+ mono-bromo splitting pattern. ¹H NMR (400 MHz, DMSO-d₆) δ 10.93 (s, 1H), 10.06 (s, 1H), 9.74 (s, 1H), 8.16 (s, 1H), 7.58 (s, 1H), 7.08 (d, J=8.3 Hz, 1H), 6.84 (d, J=8.3 Hz, 1H), 5.36 (s, 2H), 2.12 (s, 3H), 2.01 (s, 3H).

### N-(3-Methoxy-2,4,6-trimethylphenyl)-2-((1-methyl-1H-pyrazol-3-yl)amino)thiazole-5-carboxamide

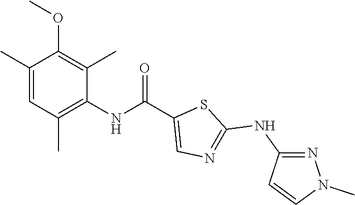

Prepared as described in Method I from potassium carbonate 325 mesh (240 mg, 1.73 mmol), Palladium (II)acetate (11 mg, 0.0495 mmol), Xantphos (57 mg, 0.0991 mmol), 1-methyl-1H-pyrazol-3-ylamine (120 mg, 1.24 mmol) and 2-bromo-N-(3-methoxy-2,4,6-trimethyl-phenyl)thiazole-5-carboxamide (200 mg, 0.495 mmol) in dry 1,4-Dioxane (10 mL) to afford the title compound (199 mg, 0.0364 mmol, 7.3% yield) as a colourless oil after purification by column chromatography over silica (40 g cartridge) eluting with a gradient of MeOH(0% to 10%; v/v) in DCM. MS (ES+) m/z=372.2 [M+H]+. ¹H NMR (400 MHz, DMSO-D6) o 11.01 (s, 1H), 9.51 (s, 1H), 8.08 (s, 1H), 7.61 (d, J=2.3 Hz, 1H), 6.94 (s, 1H), 5.94 (d, J=2.3 Hz, 1H), 3.78 (s, 3H), 3.62 (s, 3H), 2.21 (s, 3H), 2.10 (s, 3H), 2.08 (s, 3H).

### N-(3-Hydroxy-2,4,6-trimethylphenyl)-2-((1-methyl-1H-pyrazol-3-yl)amino)thiazole-5-carboxamide ( ) BAA-517

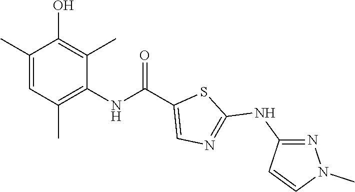

Prepared as described in Method K from N-(3-methoxy-2,4,6-trimethyl-phenyl)-2-[(1-methylpyrazol-3-yl)amino]thiazole-5-carboxamide (1.00 eq, 199 mg, 0.509 mmol) and BBr₃ (3.93 eq, 2.0 mL, 2.00 mmol) in DCM (15 mL) to afford the title compound (15 mg, 0.0420 mmol, 8.25% yield) as a white solid after HPLC purification. MS (ES+) m/z=358.1 [M+H]+. ¹H NMR (400 MHz, DMSO-D6) δ 10.98 (s, 1H), 9.45 (s, 1H), 8.07 (s, 1H), 8.06 (s, 1H), 7.61 (d, J=2.3 Hz, 1H), 6.81 (s, 1H), 5.93 (d, J=2.3 Hz, 1H), 3.78 (s, 3H), 2.15 (s, 3H), 2.05 (s, 3H), 2.02 (s, 3H).

The following example compounds were prepared similarly using Method I from appropriately substituted 2-bromo-N-(3-methoxy-phenyl)thiazole-5-carboxamide and amines followed by Method K for methoxy deprotection to phenols.

| # | Structure & Name | Analytical Data |
|---|---|---|
| BAA-516 | 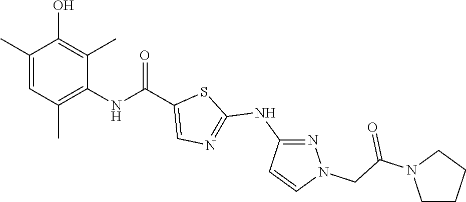 [SMILES: Cc1cc(C)c(NC(=O)c2cnc(Nc3ccn(CC(=O)N4CCCC4)n3)s2)c(C)c1O] | ¹H NMR (400 MHZ, DMSO-d₆) δ 11.01 (s, 1H), 9.46 (s, 1H), 8.08 (s, 1H), 8.06 (s, 1H), 7.59 (d, J = 2.3 Hz, 1H), 6.80 (s, 1H), 5.97 (d, J = 2.3 Hz, 1H), 4.93 (s, 2H), 3.52 (t, J = 6.8 Hz, 2H), 3.30 (t, J = 6.8 Hz, 2H), 2.14 (s, 3H), 2.04 (s, 3H), 2.02 (s, 3H), 1.91 (p, J = 6.8 Hz, 2H), 1.78 (p, J = 6.8 Hz, 2H). MS (ES⁺) m/z 455.2 (M + H)⁺. |
| | N-(3-hydroxy-2,4,6-trimethylphenyl)-2-((1-(2-oxo-2-(pyrrolidin-1-yl)ethyl)-1H- | |
| | pyrazol-3-yl)amino)thiazole-5-carboxamide | |
| BAA-547 | 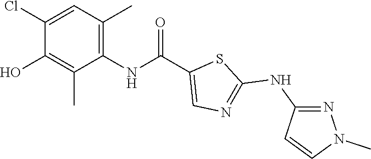 [SMILES: Cc1cc(Cl)c(O)c(C)c1NC(=O)c1cnc(Nc2ccn(C)n2)s1] | ¹H NMR (400 MHZ, DMSO-d₆) δ 11.03 (s, 1H), 9.57 (s, 1H), 8.99 (s, 1H), 8.09 (s, 1H), 7.61 (d, J = 2.2 Hz, 1H), 7.12 (s, 1H), 5.93 (d, J = 2.3 Hz, 1H), 3.79 (s, 3H), 2.13-2.01 (m, 6H). MS (ES⁺) m/z 378.2 (M + H)⁺. |
| | N-(4-chloro-3-hydroxy-2,6-dimethylphenyl)-2-((1-methyl-1H- | |
| | pyrazol-3-yl)amino)thiazole-5-carboxamide | |
| BAA-553 | 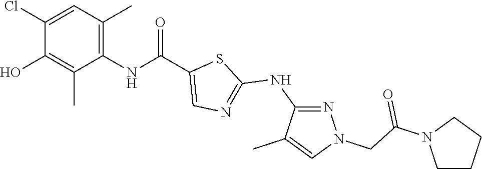 [SMILES: Cc1cn(CC(=O)N2CCCC2)nc1Nc1ncc(C(=O)Nc2c(C)cc(Cl)c(O)c2C)s1] | ¹H NMR (400 MHZ, DMSO-d₆) δ 10.72 (s, 1H), 9.55 (s, 1H), 9.00 (s, 1H), 8.10 (s, 1H), 7.40 (d, J = 1.0 Hz, 1H), 7.12 (s, 1H), 4.86 (s, 2H), 3.51 (t, J = 6.8 Hz, 2H), 3.29 (d, J = 6.9 Hz, 2H), 2.11-2.04 (m, 6H), 2.00 (d, J = 0.8 Hz, 3H), 1.96-1.85 (m, 2H), 1.83-1.71 (m, 2H). MS (ES⁺) m/z 489.3 (M + H)⁺. |
| | N-(4-chloro-3-hydroxy-2,6-dimethylphenyl)-2-((4-methyl-1-(2-oxo-2- | |
| | (pyrrolidin-1-yl)ethyl)-1H-pyrazol-3-yl)amino) thiazole-5-carboxamide | |
| BAA-565 | 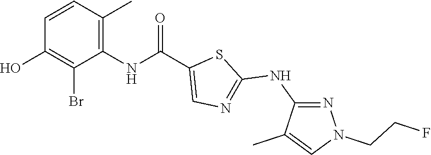 [SMILES: Cc1cn(CCF)nc1Nc1ncc(C(=O)Nc2c(C)ccc(O)c2Br)s1] | ¹H NMR (400 MHZ, DMSO-d₆) δ 10.76 (s, 1H), 10.07 (s, 1H), 9.67 (s, 1H), 8.13 (s, 1H), 7.51 (s, 1H), 7.07 (d, J = 8.4 Hz, 1H), 6.83 (d, J = 8.3 Hz, 1H), 4.75 (dt, J = 47.4, 4.7 Hz, 2H), 4.31 (dt, J= 27.7, 4.7 Hz, 2H), 2.11 (s, 3H), 2.00 (d, J = 0.8 Hz, 3H). MS (ES⁺) m/z 452.0/454.0 (M + H)⁺. |
| | N-(2-bromo-3-hydroxy-6-methylphenyl)-2-((1-(2-fluoroethyl)-4- | |
| | methyl-1H-pyrazol-3-yl) amino) thiazole-5-carboxamide | |

### Amide Formation Between Aminophenol or Protected Aminophenol and 2-Pyrazoleamine Thiazole Carboxylic Acid Derivative

### 2-((1-Methyl-1H-pyrazol-3-yl)amino)thiazole-5-carbonyl chloride

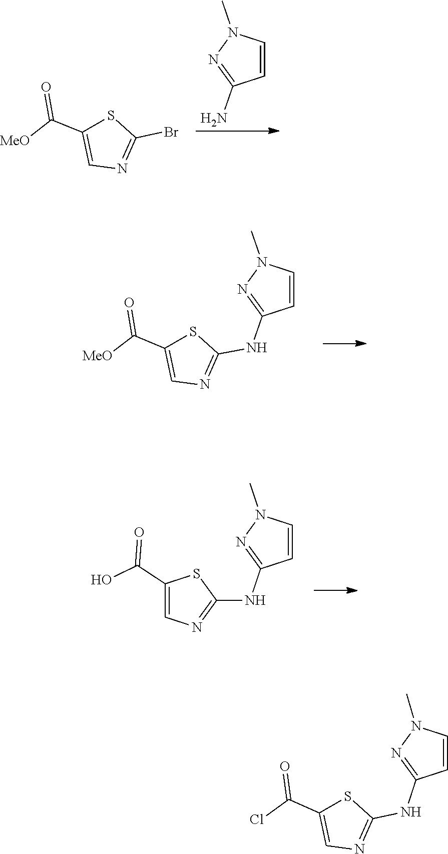

A mixture of methyl 2-bromothiazole-5-carboxylate (2.00 g, 9.01 mmol, 1.00 eq), tris(dibenzylideneacetone) dipalladium (0)(825 mg, 0.901 mmol, 0.100 eq), 4,5-bis(diphenylphospheno)-9,9-dimethylxanthene (1042 mg, 1.80 mmol, 0.200 eq), cesium carbonate
(7382 mg, 22.5 mmol, 2.50 eq), 1-methyl-1H-pyrazol-3-ylamine (2187 mg, 22.5 mmol, 2.50 eq) and 1,4-dioxane (45 mL) was degassed with nitrogen and stirred at 95° C. for 16 h. The mixture was cooled to rt. Sodium hydroxide (3693 mg, 90.1 mmol, 10.0 eq) and H₂O (45 mL) were added and the mixture was stirred at 60° C. for 2 h. After cooling to rt, the mixture was diluted with H₂O (200 mL), washed with DCM (3×150 mL). The aqueous phase was acidified to pH 1. The precipitates were filtered and dried under vacuum. The crude was dissolved in MeOH/DCM (1:1), filtered through a pad of MgSO₄, washed with more MeOH/DCM and the solvent was removed under reduced pressure to afford 2-((1-methyl-1H-pyrazol-3-yl)amino)thiazole-5-carboxylic acid (730 mg, 3.26 mmol, 36%) as light yellow solids. ¹H NMR (300 MHz, DMSO-d₆) δ 7.85 (s, 1H), 7.63 (d, J=2.3 Hz, 1H), 5.96 (d, J=2.3 Hz, 1H), 5.76 (s, 1H), 3.79 (s, 3H). MS (ES+) m/z 225.2 (M+H)⁺. Thionyl chloride (0.49 mL, 6.78 mmol, 3.00 eq) was added to a mixture of 2-[(1-methylpyrazol-3-yl)amino]thiazole-5-carboxylic acid (507 mg, 2.26 mmol, 1.00 eq), DMF (0.1 mL) and DCM (16 mL) and the mixture was stirred at rt for 3 h. The solvent was removed under reduced pressure to afford 2-[(1-methylpyrazol-3-yl)amino]thiazole-5-carbonyl chloride (693 mg, 2.86 mmol, 126%) as orange solids and was used immediately in the subsequent transformation.

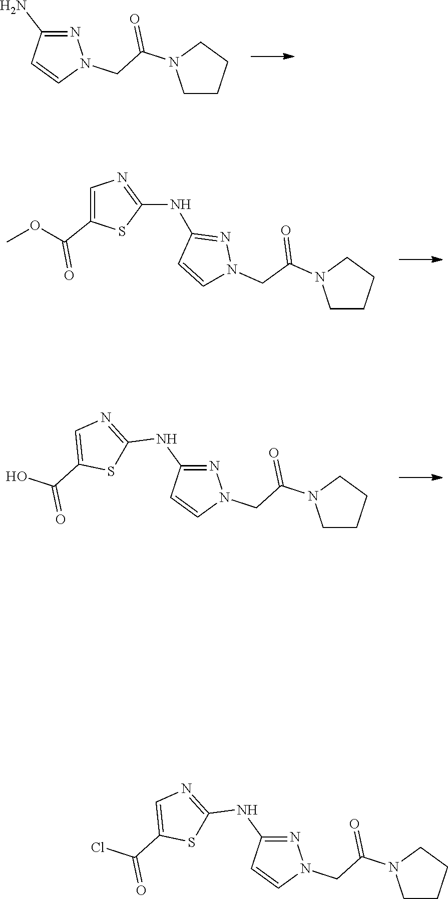 [SMILES: Nc1ccn(CC(=O)N2CCCC2)n1 ; COC(=O)c1cnc(Nc2ccn(CC(=O)N3CCCC3)n2)s1 ; O=C(O)c1cnc(Nc2ccn(CC(=O)N3CCCC3)n2)s1 ; O=C(Cl)c1cnc(Nc2ccn(CC(=O)N3CCCC3)n2)s1]

Step 1. 4,5-Bis(diphenylphosphino)-9,9-dimethylxanthene (Xantphos)(515 mg, 0.864 mmol) and tris(dibenzylideneacetone) dipalladium (0)(Pd2(dba)3)(400 mg, 0.437 mmol) were added into the solution of caesium carbonate (5.20 g, 16.0 mmol), methyl 2-bromothiazole-5-carboxylate (2.65 g, 11.9 mmol) and 2-(3-aminopyrazol-1-yl)-1-pyrrolidin-1-yl-ethanone (2.50 g, 12.9 mmol) in dry 1,4-dioxane (75 mL) under N₂ and the reaction mixture was heated in a sealed tube at 80° C. for 18 h, cooled down to rt and concentrated in vacuo. The crude was dissolved in DCM, the precipitate formed was recovered by filtration and dried to provide methyl 2-[1-(2-oxo-2-pyrrolidin-1-yl-ethyl)pyrazol-3-yl]amino]thiazole-5-carboxylate (2228 mg, 6.18 mmol, 51.77% yield) as a brown solid. MS (ES+) m/z=336.0 [M+H]+, ¹H NMR (400 MHz, DMSO-D6) δ 7.95 (s, 1H), 7.61 (d, J=2.3 Hz, 1H), 5.96 (d, J=2.3 Hz, 1H), 4.95 (s, 2H), 3.76 (s, 3H), 3.55 (t, J=6.9 Hz, 2H), 3.31 (d, J=6.9 Hz, 2H), 1.98-1.89 (m, 2H), 1.85-1.74 (m, 2H).

Step 2. Lithium hydroxide, monohydrate (531 mg, 7.08 mmol) was added into the solution of methyl 2-[1-(2-oxo-2-pyrrolidin-1-yl-ethyl)pyrazol-3-yl]amino]thiazole-5-carboxylate (2128 mg, 5.90 mmol) in THF (30 mL) and Water (30 mL) at rt and stirred at 65° C. for 1.5 h. The reaction was cooled down to rt and THF was evaporated. The remining water layer was extracted with EtOAc (3× 30 mL) and adjusted at pH=5-6 with 1N HCl solution. The solution was left standing overnight. A ppt appeared which was filtered off and dried to provide 2-[1-(2-oxo-2-pyrrolidin-1-yl-ethyl)pyrazol-3-yl]amino]thiazole-5-carboxylic acid (276 mg, 0.678 mmol, 11.50% yield) as a white solid. MS (ES+) m/z=322.3 [M+H]+. ¹H NMR (400 MHz, DMSO-D6) δ 11.24 (s, 1H), 7.86 (d, J=1.0 Hz, 1H), 7.61 (d, J=2.3 Hz, 1H), 5.97 (d, J=2.3 Hz, 1H), 4.95 (s, 2H), 3.53 (t, J=6.8 Hz, 2H), 3.32 (t, J=6.9 Hz, 2H), 1.96-1.87 (m, 2H), 1.85-1.74 (m, 2H).

Step 3. Thionyl chloride (6.60 eq, 0.15 mL, 2.06 mmol) and N, N-dimethylformamide, (2.08 eq, 50 μL, 0.646 mmol) were added to a suspension of 2-[1-(2-oxo-2-pyrrolidin-1-yl-ethyl)pyrazol-3-yl]amino]thiazole-5-carboxylic acid (1.00 eq, 100 mg, 0.311 mmol) in DCM (10 mL). After 3 h the volatiles are evaporated to afford 2-((1-(2-oxo-2-(pyrrolidin-1-yl)ethyl)-1H-pyrazol-3-yl)amino)thiazole-5-carbonyl chloride that is used in the next step without purification.

### General Method Y²: Amide Formation Between Silylated Aminophenol and 2-Pyrazole-Amine Thiazole Carboxylic Acid Derivative

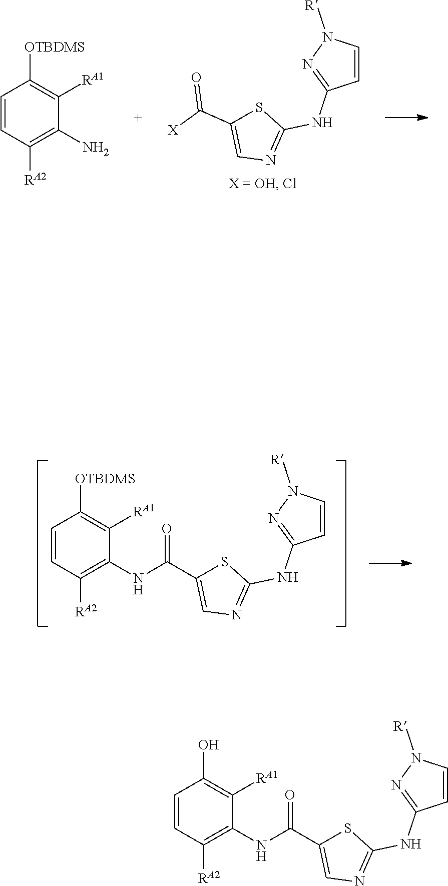

A mixture of silylated aminophenol (1 eq) and caesium carbonate (2 eq) in dry 1,4-dioxane and MeCN under N₂ is heated at 90° C. for 5-10 minutes. A solution of acid chloride in in dry 1,4-dioxane and MeCN is added dropwise at 90° C. and the reaction mixture is stirred for 18 h, then cooled down and concentrated hydrogen chloride solution (6 eq) is added and the reaction mixture is stirred at rt for 2 h. The reaction mixture is concentrated to dryness and the crude material purified by reverse phase chromatography reverse phase chromatography (C₁₈) using a gradient of MeCN: H₂O (0.1 formic acid) and/or by preparative HPLC-MS using a gradient of high or low pH aq. MeCN to afford the phenol product.

### N-(6-Chloro-3-hydroxy-2-methyl-phenyl)-2-[(1-methylpyrazol-3-yl)amino]thiazole-5-carboxamide( ) BAA-474

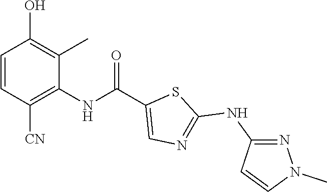 [SMILES: Cc1c(O)ccc(C#N)c1NC(=O)c1cnc(Nc2ccn(C)n2)s1]

Prepared as described in Method Y² from caesium carbonate (144 mg, 0.441 mmol 3-[tert-butyl(dimethyl) silyl]oxy-6-chloro-2-methyl-aniline (75 mg, 0.221 mmol) in dry 1,4-dioxane (1 mL) and MeCN (1 mL) and 2-[(1-methylpyrazol-3-yl)amino]thiazole-5-carbonyl chloride; hydrochloride
(100 mg, 0.279 mmol) in dry MeCN (0.5 mL) and 1,4-dioxane (0.5 mL) to afford the title compound
(7.0 mg, 0.0189 mmol, 8.54% yield) as a white solid after chromatography over C18 (23 g cartridge) eluting with a gradient of MeCN (0.1% formic acid)(5% to 95%; v/v) in water (0.1% formic acid) and repurification by prep HPLC eluting with a gradient of MeCN (5% to 44.5%; v/v) in NH₄COO-(0.1% formic acid). MS (ES+) m/z=364.0/366.0 [M+H]+. ¹H NMR (400 MHz, DMSO-D6) δ 9.73 (s, 2H), 8.11 (s, 1H), 7.61 (d, J=2.3 Hz, 1H), 7.16 (d, J=8.7 Hz, 1H), 6.80 (d, J=8.7 Hz, 1H), 5.94 (d, J=2.3 Hz, 1H), 3.79 (s, 3H), 2.01 (s, 3H).

The following example compounds were prepared similarly using Method Y² with appropriate silylated aminophenols.

| # | Structure & Name | Analytical Data |
|---|---|---|
| BAA-468 | 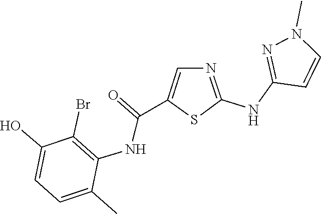 [SMILES: Cc1ccc(O)c(Br)c1NC(=O)c1cnc(Nc2ccn(C)n2)s1] | MS (ES+) m/z 408.1/410.2 [M+H]+ mono Br pattern. ¹H NMR, (400 MHZ, DMSO- D6) δ 11.04 (s, 1H), 10.05 (s, 1H), 9.70 (s, 1H), 8.11 (s, 1H), 7.61 (d, J = 2.3 Hz, 1H), 7.07 (d, J = 8.4 Hz, 1H), 6.84 (d, J = 8.3 Hz, 1H), 5.94 (d, J = 2.3 Hz, 1H), 3.79 (s, 3H), 2.12 (s, 3H) |
| | N-(2-bromo-3-hydroxy-6-methyl-phenyl)-2-[(1- | |
| | methylpyrazol-3-yl)amino]thiazole-5-carboxamide | |
| BAA-498 | 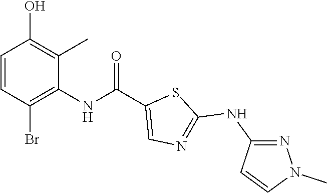 [SMILES: Cc1c(O)ccc(Br)c1NC(=O)c1cnc(Nc2ccn(C)n2)s1] | MS (ES+) m/z = 408.1/410.1 [M+H]+ mono Br pattern. ¹H NMR (400 MHZ, DMSO-D6) δ 11.04 (s, 1H), 9.75 (d, J = 6.3 Hz, 2H), 8.11 (s, 1H), 7.61 (d, J = 2.3 Hz, 1H), 7.31 (d, J = 8.6 Hz, 1H), 6.75 (d, J = 8.7 Hz, 1H), 5.94 (d, J = 2.3 Hz, 1H), 3.79 (s, 3H), 2.03 (s, 3H). |
| | N-(6-bromo-3-hydroxy-2-methylphenyl)-2-((1-methyl-1H- | |
| | pyrazol-3-yl)amino)thiazole-5-carboxamide | |
| BAA-476 | 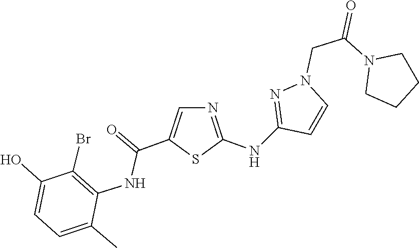 [SMILES: Cc1ccc(O)c(Br)c1NC(=O)c1cnc(Nc2ccn(CC(=O)N3CCCC3)n2)s1] | MS (ES+) m/z = 505.1 /507.2 [M+H]+ mono Br pattern. ¹H NMR (400 MHZ, DMSO-D6) δ 11.05 (s, 1H), 10.09 (s, 1H), 9.71 (s, 1H), 8.12 (s, 1H), 7.60 (d, J = 2.3 Hz, 1H), 7.07 (d, J = 8.4 Hz, 1H), 6.84 (d, J = 8.3 Hz, 1H), 5.97 (d, J = 2.3 Hz, 1H), 4.93 (s, 2H), 3.52 (t, J = 6.8 Hz, 2H), 3.30 (t, J = 6.9 Hz, 2H), 2.11 (s, 3H), 1.91 (p, J = 6.8 Hz, 2H), 1.78 (p, J = 6.8 Hz, 2H). |
| | N-(2-bromo-3-hydroxy-6-methyl-phenyl)-2-[1-(2-oxo- | |
| | 2-pyrrolidin-1-yl-ethyl)pyrazol-3-yl]amino]thiazole-5-carboxamide | |

### General Method Y³: Amide Formation Between Amino-Methoxy-Aryl and 2-Pyrazole-Amine Thiazole Carboxylic Acid Derivative and Deprotection of Methoxy to Phenol

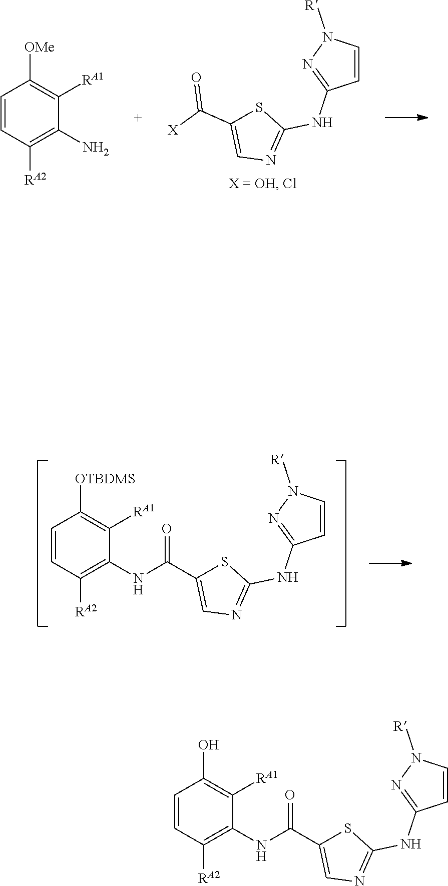

A mixture of methoxyaniline (1.2 eq) and caesium carbonate (2 eq) in 1,4-dioxane and MeCN under N₂ is heated at 90° C. for 5-10 minutes. A solution of acid chloride in in dry 1,4-dioxane and MeCN is added dropwise at 90° C. and the reaction mixture is stirred for 2 h at 90° C. and for 16 h at rt. The reaction mixture is diluted with EtOAc and washed with water, brine, dried over sodium sulfate filtered and evaporated. The crude material is purified by reverse phase chromatography reverse phase chromatography (C18) using a gradient of MeCN: H₂O (0.1 formic acid) and/or by preparative HPLC-MS using a gradient of high or low pH aq. MeCN. The methoxy product is suspended in DCM, BBr₃ (9 eq) is added and the reaction mixture is stirred for 3 h, then is quenched with MeOH, evaporated and the crude material is purified by reverse phase chromatography to afford the phenol product.

### N-(2-Ethyl-3-hydroxy-6-methylphenyl)-2-((1-methyl-1H-pyrazol-3-yl)amino)thiazole-5-carboxamide( ) BAA-469

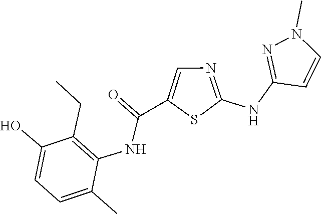 [SMILES: CCc1c(O)ccc(C)c1NC(=O)c1cnc(Nc2ccn(C)n2)s1]

Prepared as described in Method Y³ from 2-ethyl-3-methoxy-6-methyl-aniline (1.2 eq, 100 mg, 0.605 mmol) and caesium carbonate (2.03 eq, 330 mg, 1.01 mmol) and 2-((1-methyl-1H-pyrazol-3-yl)amino)thiazole-5-carbonyl chloride (1 eq) in 1,4-dioxane (2 mL) and MeCN (2 mL), followed by BBr₃ (8.84 eq, 2.0 mL, 2.00 mmol) to afford the title compound (35 mg, 0.0960 mmol, 42.4% yield) after purification by column chromatography over C18 (23 g cartridge) eluting with a gradient of MeCN (0.1% formic acid)(5% to 95%; v/v) in water (0.1% formic acid). MS (ES+) m/z=358.3 [M+H]+. ¹H NMR (400 MHz, DMSO-D6) o 11.00 (s, 1H), 9.45 (s, 1H), 9.13 (s, 1H), 8.08 (s, 1H), 7.61 (d, J=2.2 Hz, 1H), 6.88 (d, J=8.3 Hz, 1H), 6.68 (d, J=8.1 Hz, 1H), 5.93 (d, J=2.3 Hz, 1H), 3.78 (s, 3H), 2.57-2.50 (m, 2H), 2.05 (s, 3H), 0.99 (t, J=7.4 Hz, 3H).

The following example compounds were prepared similarly using Method Y³ with appropriate methoxyanilines.

| # | Structure & Name | Analytical Data |
|---|---|---|
| BAA-249 | 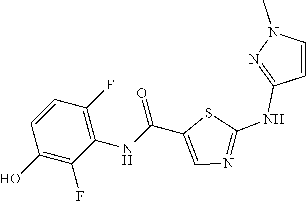 [SMILES: Cn1ccc(Nc2ncc(C(=O)Nc3c(F)ccc(O)c3F)s2)n1] | ¹H NMR (300 MHZ, Methanol- d₄/chloroform-d) δ 8.01 (s, 1H), 7.40 (d, J = 2.3 Hz, 1H), 6.87-6.78 (m, 2H), 6.02 (d, J = 2.3 Hz, 1H), 3.83 (s, 3H). 19F NMR (282 MHZ, Methanol-d₄/ chloroform-d) −131.40 (d, J = 1.5 Hz), −141.28 (d, J = 1.5 Hz). MS (ES+) m/z 352.2 (M + H)⁺ |
| | N-(2,6-difluoro-3-hydroxyphenyl)-2-((1-methyl-1H- | |
| | pyrazol-3-yl) amino)thiazole-5-carboxamide | |
| BAA-475 | 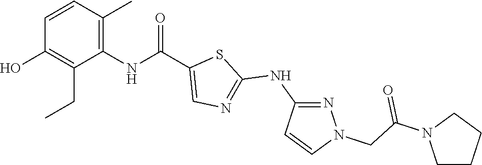 [SMILES: CCc1c(O)ccc(C)c1NC(=O)c1cnc(Nc2ccn(CC(=O)N3CCCC3)n2)s1] | ¹H NMR (400 MHZ, DMSO-d₆) δ 11.03 (s, 1H), 9.46 (s, 1H), 9.13 (s, 1H), 8.09 (s, 1H), 7.59 (d, J = 2.4 Hz, 1H), 6.88 (d, J = 8.2 Hz, 1H), 6.68 (d, J = 8.2 Hz, 1H), 5.97 (d, J = 2.3 Hz, 1H), 4.93 (s, 2H), 3.52 (t, J = 6.7 Hz, 2H), 3.31 (t, J = 6.9 Hz, 2H), 2.04 (s, 3H), 1.91 (p, J = 6.7 Hz, 2H), 1.78 (p, J = 6.8 Hz, 2H), 0.99 (t, J = 7.4 Hz, 3H). 2H of methylene of Et under DMSO peak at 2.50 ppm. MS (ES⁺) m/z 455.3 (M + H)⁺. |
| | N-(2-ethyl-3-hydroxy-6-methylphenyl)-2-((1-(2-oxo-2-(pyrrolidin- | |
| | 1-yl)ethyl)-1H-pyrazol-3-yl)amino) thiazole-5-carboxamide | |

### N-(2-Chloro-6-fluoro-3-methoxyphenyl)-2-((1-methyl-1H-pyrazol-3-yl)amino)thiazole-5-carboxamide

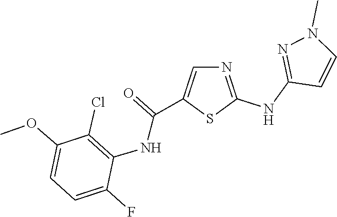 [SMILES: COc1ccc(F)c(NC(=O)c2cnc(Nc3ccn(C)n3)s2)c1Cl]

Thionyl chloride (3.57 eq, 100 μL, 1.37 mmol) and N, N-dimethylformamide, (0.337 eq, 10 μL, 0.129 mmol) were added to a suspension of 2-[(1-methylpyrazol-3-yl)amino]thiazole-5-carboxylic acid; hydrochloride (1.00 eq, 100 mg, 0.384 mmol) in DCM (10 mL). After 2 hours the volatiles are evaporated. In a separate flask, a mixture of 2-chloro-6-fluoro-3-methoxyaniline (1.48 eq, 100 mg, 0.570 mmol) and caesium carbonate (2.00 eq, 250 mg, 0.767 mmol) in 1,4-Dioxane (2 mL) and MeCN (2 mL) was warmed at 90° C., for 10 min. The acid chloride formed above was added. The heating was continued at 90° C. for 2 h, then the reaction mixture was stirred at ambient temperature overnight and evaporated. The crude material was purified by column chromatography over C18 (23 g cartridge) eluting with a gradient of MeCN (0.1% formic acid)(5% to 95%; v/v) in water (0.1% formic acid) [note 5] to afford the title compound (63 mg, 0.165 mmol, 43.02% yield) as an off-white solid. MS (ES+) m/z=382.0/384.0 [M+H]+ mono CI pattern. ¹H NMR
(400 MHz, DMSO-D6) δ 11.12 (s, 1H), 9.94 (s, 1H), 8.14 (d, J=7.4 Hz, 1H), 7.62 (d, J=2.3 Hz, 1H), 7.30 (t, J=9.2 Hz, 1H), 7.14 (dd, J=9.4, 4.6 Hz, 1H), 5.94 (d, J=2.3 Hz, 1H), 3.87 (s, 3H), 3.79 (s, 3H). ¹⁹F NMR (376 MHz, DMSO-D6) δ-126.33 (dd, J=9.2, 4.6 Hz).

### 2-((4-Bromo-1-methyl-1H-pyrazol-3-yl)amino)-N-(2-chloro-6-fluoro-3-hydroxyphenyl)thiazole-5-carboxamide( ) BAA-472

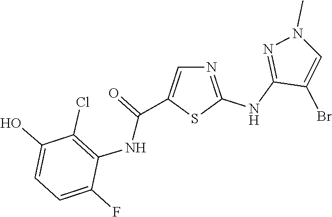 [SMILES: Cn1cc(Br)c(Nc2ncc(C(=O)Nc3c(F)ccc(O)c3Cl)s2)n1]

BBr_{3 1}M DCM (3.03 eq, 0.5 mL, 0.5 mmol) was added to a suspension of N-(2-chloro-6-fluoro-3-methoxy-phenyl)-2-[(1-methylpyrazol-3-yl)amino]thiazole-5-carboxamide (1 eq, 63 mg, 0.165 mmol) in DCM (10 mL) under nitrogen. The reaction mixture was stirred for 4 h, then further BBr_{3 1}M DCM (1.5 mL) was added, and the stirring was continued for 16 h at rt. The reaction was quenched by addition of MeOH(5 mL) and water (1 mL). The solvent was evaporated, and the crude material was purified by column chromatography over C18 (23 g cartridge) eluting with a gradient of MeCN (0.1% formic acid)(5% to 95%; v/v) in water (0.1% formic acid). The product was triturated in TBME, filtrated and the solid dried to afford the title compound (50 mg, 0.104 mmol, 63.09% yield) as a white solid.MS (ES+) m/z=446.0/448/0/450.0 [M+H]+ mono Br/mono CI pattern. ¹H NMR (400 MHz, DMSO-D6) δ 10.82 (s, 1H), 10.25 (s, 1H), 9.88 (s, 1H), 8.11 (s, 1H), 7.92 (s, 1H), 7.13 (t, J=9.2 Hz, 1H), 6.93 (dd, J=9.1, 4.9 Hz, 1H), 3.81 (s, 3H). ¹⁹F NMR (376 MHz, DMSO-D6) δ-128.17 (dd, J=9.1, 4.8 Hz).

### N-(2-Chloro-6-fluoro-3-hydroxyphenyl)-2-((1-methyl-1H-pyrazol-3-yl)amino)thiazole-5-carboxamide ( ) BAA-480

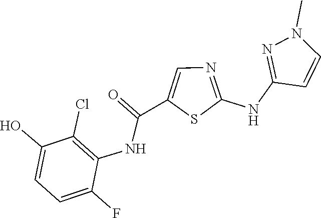 [SMILES: Cn1ccc(Nc2ncc(C(=O)Nc3c(F)ccc(O)c3Cl)s2)n1]

Palladium hydroxide on carbon (Pd(OH) 2/C, 20% wt)(0.454 eq, 40 mg, 0.0570 mmol) as a slurry in MeCN (2 mL) was added under nitrogen to a solution of 2-[(4-bromo-1-methyl-pyrazol-3-yl)amino]-N-(2-chloro-6-fluoro-3-hydroxy-phenyl)thiazole-5-carboxamide (1.00 eq, 56 mg, 0.125 mmol) in MeCN (5 mL) and water (5 mL). The reaction mixture was stirred under a hydrogen atmosphere for 4 hours, then the catalyst was filtered off over celite and the filtrate evaporated. The resultant solid was triturated in TBME and collected by filtration. The crude material was purified by column chromatography over C18 (23 g cartridge) eluting with a gradient of MeCN
(0.1% formic acid)(5% to 70%; v/v) in water (0.1% formic acid) to afford the title compound (26 mg, 0.0693 mmol, 55.3% yield) as a white solid. MS (ES+) m/z=368.1/370.1 [M+H]+ mono Cl pattern. ¹H NMR (400 MHz, DMSO-D6) o 11.10 (s, 1H), 10.23 (s, 1H), 9.85 (s, 1H), 8.12 (s, 1H), 7.62 (d, J=2.2 Hz, 1H), 7.12 (t, J=9.1 Hz, 1H), 6.93 (dd, J=9.1, 4.9 Hz, 1H), 5.94 (d, J=2.2 Hz, 1H), 3.79 (s, 3H). ¹⁹F NMR (376 MHz, DMSO-D6) δ-128.13 (dd, J=9.2, 4.9 Hz).

### N-(6-Chloro-2-fluoro-3-methoxyphenyl)-2-((1-methyl-1H-pyrazol-3-yl)amino)thiazole-5-carboxamide

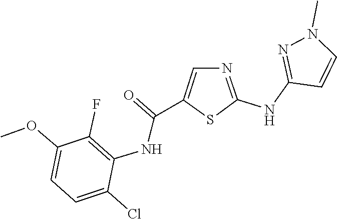 [SMILES: COc1ccc(Cl)c(NC(=O)c2cnc(Nc3ccn(C)n3)s2)c1F]

Thionyl chloride (3.57 eq, 0.10 mL, 1.37 mmol) and N, N-dimethylformamide, (0.337 eq, 10 uL, 0.129 mmol) were added to a suspension of 2-[(1-methylpyrazol-3-yl)amino]thiazole-5-carboxylic acid; hydrochloride (1.00 eq, 100 mg, 0.384 mmol) in DCM (10 mL). After 2 hours the volatiles were evaporated. In a separate flask, a mixture of 6-chloro-2-fluoro-3-methoxyaniline
(1.26 eq, 85 mg, 0.484 mmol) and caesium carbonate (2.00 eq, 250 mg, 0.767 mmol) in 1,4-dioxane (2 mL) and MeCN (2 mL) was warmed at 90° C., for 10 min. The acid chloride formed above was added, and the heating at 90° C. was continued for 2 h, then the reaction mixture was stirred at ambient temperature overnight and evaporated. The crude material was purified by column chromatography over C18 (23 g cartridge) eluting with a gradient of MeCN (0.1% formic acid)(5% to 95%; v/v) in water (0.1% formic acid) to afford the title compound (77 mg, 0.182 mmol, 47.32% yield) as an off-white solid. MS (ES+) m/z=382.0/384.0 [M+H]+ mono CI pattern. ¹H NMR
(400 MHz, DMSO-D6) δ 11.13 (s, 1H), 9.95 (s, 1H), 8.13 (s, 1H), 7.62 (d, J=2.2 Hz, 1H), 7.35 (dd, J=9.1, 2.0 Hz, 1H), 7.18 (t, J=8.9 Hz, 1H), 5.94 (d, J=2.3 Hz, 1H), 3.88 (s, 3H), 3.79 (s, 3H).

### 2-((4-Bromo-1-methyl-1H-pyrazol-3-yl)amino)-N-(6-chloro-2-fluoro-3-hydroxyphenyl)thiazole-5-carboxamide( ) BAA-471

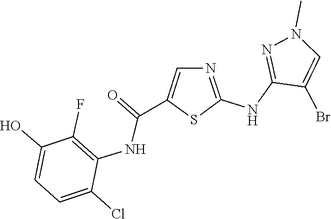 [SMILES: Cn1cc(Br)c(Nc2ncc(C(=O)Nc3c(Cl)ccc(O)c3F)s2)n1]

BBr_{3 1}M DCM (2.87 eq, 0.50 mL, 0.500 mmol) was added to a suspension of N-(6-chloro-2-fluoro-3-methoxy-phenyl)-2-[(1-methylpyrazol-3-yl)amino]thiazole-5-carboxamide (1 eq, 74 mg, 0.174 mmol) in DCM (10 mL) under nitrogen. The reaction mixture was stirred for 4 h, then further BBr_{3 1}M DCM (1.5 mL) was added, and the stirring was continued for 16 h at rt. The reaction was quenched by addition of MeOH(5 mL) and water (1 mL). The solvent was evaporated, and the crude material was purified by column chromatography over C18 (23 g cartridge) eluting with a gradient of MeCN (0.1% formic acid)(5% to 95%; v/v) in water (0.1% formic acid). The product was triturated in TBME, filtrated and the solid dried to afford the title compound (57 mg, 0.121 mmol, 69.50% yield) as a white solid. MS (ES+) m/z=446.0/448/0/450.0 [M+H]+ mono Br/mono CI pattern. ¹H NMR (400 MHz, DMSO-D6) δ 10.79 (s, 1H), 10.27 (s, 1H), 9.90 (s, 1H), 8.12 (s, 1H), 7.93 (s, 1H), 7.18 (dd, J=8.9, 1.8 Hz, 1H), 6.94 (t, J=8.9 Hz, 1H), 3.81 (s, 3H). ¹⁹F NMR (376 MHz, DMSO-D6) δ-137.52 (d, J=8.8 Hz).

### N-(6-Chloro-2-fluoro-3-hydroxyphenyl)-2-((1-methyl-1H-pyrazol-3-yl)amino)thiazole-5-carboxamide( ) BAA-479

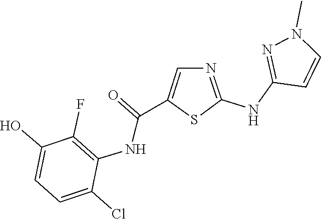 [SMILES: Cn1ccc(Nc2ncc(C(=O)Nc3c(Cl)ccc(O)c3F)s2)n1]

Boron tribromide 1M DCM (6.43 eq, 1.0 mL, 1.00 mmol) was added to a suspension of N-(6-chloro-2-fluoro-3-methoxy-phenyl)-2-[(1-methylpyrazol-3-yl)amino]thiazole-5-carboxamide (1.00 eq, 66 mg, 0.156 mmol) in DCM (10 mL) under nitrogen and the reaction mixture was stirred for 18 h, then quenched with MeOH(5 mL) and evaporated. The crude material was purified by column chromatography over C18 (23 g cartridge) eluting with a gradient of MeCN (0.1% formic acid)(5% to 95%; v/v) in water (0.1% formic acid) to afford the title compound (34 mg, 0.0924 mmol, 59.42% yield) as a white solid. MS (ES+) m/z=368.1/370.1 [M+H]+ mono CI pattern. ¹H NMR (400 MHz, DMSO-D6) δ 11.11 (s, 1H), 10.27 (s, 1H), 9.87 (s, 1H), 8.12 (s, 1H), 7.62 (d, J=2.2 Hz, 1H), 7.18 (dd, J=8.9, 1.9 Hz, 1H), 6.94 (t, J=8.9 Hz, 1H), 5.94 (d, J=2.3 Hz, 1H), 3.79 (s, 3H). ¹⁹F NMR
(376 MHz, DMSO-D6) δ-137.50 (d, J=9.1 Hz).

### Synthesis of Reference Compounds

### 2-Amino-N-(5-hydroxy-2-methyl-phenyl)thiazole-5-carboxamide (REF-001)

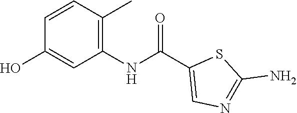 [SMILES: Cc1ccc(O)cc1NC(=O)c1cnc(N)s1]

Step 1. To a stirring solution of 2-N-Boc-Amino-thiazole-5-carboxylic acid (100 mg, 0.409 mmol, 1.00 eq) and 3-amino-4-methylphenol (60 mg, 0.491 mmol, 1.20 eq) in THF (5 mL) was added N, N-diisopropylethylamine (0.14 mL, 0.819 mmol, 2.00 eq) and 1-propanephosphonic anhydride 50% in Ethyl Acetate (0.36 mL, 0.614 mmol, 1.50 eq). The reaction mixture was heated to 65° C. and left to stir overnight. The reaction mixture was cooled to room temperature, water and EtOAc added and the layers separated. The aqueous layer was extracted twice more with EtOAc, the organics combined, passed through a phase separator and the solvents removed in vacuo to afford the crude material as an orange solid, which was purified by flash column chromatography
(0-100% EtOAc in pet. ether) to afford tert-butyl N-[5-[(5-hydroxy-2-methyl-phenyl) carbamoyl]thiazol-2-yl]carbamate (115 mg, 0.329 mmol, 80%) as an off-white solid. MS (ES+) m/z 350.0 (M+H). ¹H NMR (300 MHz, DMSO-d₆) δ 11.79 (s, 1H), 9.65 (s, 1H), 9.25 (s, 1H), 8.16 (s, 1H), 7.03 (d, J=8.2 Hz, 1H), 6.79 (d, J=2.5 Hz, 1H), 6.57 (dd, J=8.2, 2.6 Hz, 1H), 2.11 (s, 3H), 1.51 (s, 9H).

Step 2. To a stirring suspension of tert-butyl N-[5-[(5-hydroxy-2-methyl-phenyl) carbamoyl]thiazol-2-yl]carbamate (50%, 115 mg, 0.165 mmol, 1.00 eq) in methanol (2 mL) was added hydrochloric acid (4M in 1,4-dioxane, 0.62 mL, 2.47 mmol, 15.0 eq). The yellow reaction mixture was warmed to 40° C. and left to stir overnight. The solvents were removed in vacuo to afford the crude product, which was purified by preparative LCMS (high pH), fractions containing product were collected and loaded onto a pre-quilibrated SCX column (washing with MeOH and eluting with 7NH₃/MeOH) to afford the title compound (7.7 mg, 19%) as a white solid. MS (ES+) m/z 249.9 (M+H). ¹H NMR (300 MHz, DMSO-d₆) δ 9.31 (br s, 1H), 9.21 (s, 1H), 7.81 (s, 1H), 7.56 (br s, 2H), 7.00 (d, J=8.2 Hz, 1H), 6.76 (d, J=2.6 Hz, 1H), 6.54 (dd, J=8.2, 2.6 Hz, 1H), 2.09 (s, 3H).

### 2-Amino-N-(3-hydroxy-2-methyl-phenyl)thiazole-5-carboxamide (REF-002)

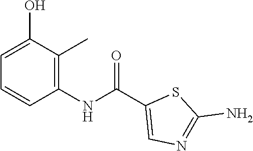 [SMILES: Cc1c(O)cccc1NC(=O)c1cnc(N)s1]

Step 1. To a stirring solution of 2-N-Boc-Amino-thiazole-5-carboxylic acid (100 mg, 0.409 mmol, 1.00 eq) and 3-amino-2-methylphenol (60 mg, 0.491 mmol, 1.20 eq) in THF (5 mL) was added N, N-diisopropylethylamine (0.14 mL, 0.819 mmol, 2.00 eq) and 1-propanephosphonic anhydride 50% in Ethyl Acetate (0.36 mL, 0.614 mmol, 1.50 eq). The reaction mixture was heated to 65° C. and left to stir over the weekend. The reaction mixture was cooled to room temperature, water and EtOAc added and the layers separated. The aqueous layer was extracted twice more with EtOAc, the organics combined, passed through a phase separator and the solvents removed in vacuo to afford the crude material as a yellow solid. The crude material was taken up in MeOH and the resulting solids collected by filtration, washing with small amounts of MeOH to afford the desired product tert-butyl N-[5-[(3-hydroxy-2-methyl-phenyl) carbamoyl]thiazol-2-yl]carbamate (48 mg, 0.136 mmol, 33%) as an off-white solid. MS (ES+) m/z 349.9 (M+H). ¹H NMR (300 MHz, DMSO-d₆) δ 11.77 (br s, 1H), 9.77 (s, 1H), 8.16 (s, 1H), 7.03-6.94 (m, 1H), 6.79-6.68 (m, 2H), 2.01 (s, 3H), 1.51 (s, 9H).

Step 2. To a stirring suspension of tert-butyl N-[5-[(3-hydroxy-2-methyl-phenyl) carbamoyl]thiazol-2-yl]carbamate (50%, 48 mg, 0.0680 mmol, 1.00 eq) in methanol (2 mL) was added hydrochloric acid (4M in 1,4-dioxane, 0.25 mL, 1.02 mmol, 15.0 eq). The yellow reaction mixture was warmed to 40° C. and left to stir for 4 days. The solvents were removed in vacuo to afford the crude product which was purified by preparative LCMS (high pH), fractions containing product were collected and loaded onto a pre-quilibrated SCX column (washing with MeOH and eluting with 7NH₃/MeOH) to afford the title compound (3.4 mg, 20%) as an off-white solid. MS (ES+) m/z 249.9 (M+H). ¹H NMR (300 MHz, DMSO-d₆) δ 9.43 (br s, 1H), 9.35 (s, 1H), 7.81 (s, 1H), 7.54 (br s, 2H), 7.00-6.92 (m, 1H), 6.75-6.66 (m, 2H), 1.99 (s, 3H).

### N-(2,6-Dimethylphenyl)-2-[(1-methylpyrazol-3-yl)amino]thiazole-5-carboxamide (REF-003)

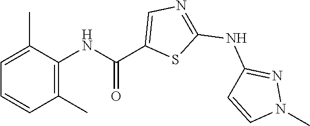 [SMILES: Cc1cccc(C)c1NC(=O)c1cnc(Nc2ccn(C)n2)s1]

Step 1. To a stirring solution of 2-bromo-1,3-thiazole-5-carboxylic acid (500 mg, 2.40 mmol, 1.00 eq) and 2,6-Dimethylaniline (0.36 mL, 2.88 mmol, 1.20 eq) in THF (15 mL) was added N,N-diisopropylethylamine (0.84 mL, 4.81 mmol, 2.00 eq) and 1-propane phosphonic anhydride 50% in Ethyl Acetate (2.1 mL, 3.61 mmol, 1.50 eq). The reaction mixture was heated to 65° C. and left to stir overnight. The mixture was cooled to room temperature, water and EtOAc added and the layers separated. The aqueous layer was extracted twice more with EtOAc. The organic fractions were collected, passed through a phase separator and the solvents removed in vacuo to afford the crude product as an orange oil. The crude material was purified by flash column chromatography
(0-100% EtOAc in Pet. Ether) to afford 2-bromo-N-(2,6-dimethylphenyl)thiazole-5-carboxamide
(652 mg, 2.10 mmol, 87%) as an off-white solid. MS (ES+) m/z 310.9, 312.8 (M+H)—Br isotope pattern. ¹H NMR (300 MHz, DMSO-d₆) δ 10.10 (s, 1H), 8.44 (s, 1H), 7.19-7.10 (m, 3H), 2.19 (s, 6H).

Step 2. A stirring mixture of 2-bromo-N-(2,6-dimethylphenyl)thiazole-5-carboxamide (75 mg, 0.241 mmol, 1.00 eq), 1-methyl-1H-pyrazol-3-ylamine (0.021 mL, 0.289 mmol, 1.20 eq) and cesium carbonate (158 mg, 0.482 mmol, 2.00 eq) in 1,4-dioxane (4 mL) was flushed with nitrogen before the addition of tris(dibenzylideneacetone) dipalladium (0)(11 mg, 0.0121 mmol, 0.0500 eq) and 4,5-bis(diphenylphospheno)-9,9-dimethylxanthene (14 mg, 0.0241 mmol, 0.1000 eq). The reaction mixture was heated to 100° C. and left to stir overnight, then cooled to room temperature and the solvents removed in vacuo to afford crude material which was purified by preparative LCMS (high pH) to afford the title product (8.6 mg, 11%) as a pale pink solid. MS (ES+) m/z 328.0 (M+H). ¹H NMR (300 MHz, DMSO-d₆) δ 11.02 (s, 1H), 9.54 (s, 1H), 8.10 (s, 1H), 7.62 (d, J=2.2 Hz, 1H), 7.15-7.04 (m, 3H), 5.95 (d, J=2.3 Hz, 1H), 3.80 (s, 3H), 2.19 (s, 6H).

### N-(3-Methoxy-2,6-dimethyl-phenyl)-2-[(1-methylpyrazol-3-yl)amino]thiazole-5-carboxamide (REF-004)

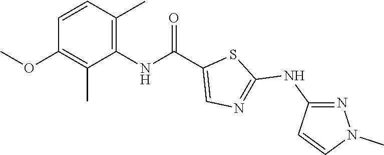

Argon was bubbled through the suspension of 2-bromo-N-(3-methoxy-2,6-dimethyl-phenyl)thiazole-5-carboxamide (150 mg, 0.440 mmol, 1.00 eq), sodium tert-butoxide (148 mg, 1.54 mmol, 3.50 eq) and 1-methyl-1H-pyrazol-3-ylamine (64 mg, 0.659 mmol, 1.50 eq) in 1,4-dioxane
(25 mL) for 10 minutes, followed by the addition of 4,5-bis(diphenyl phospheno)-9,9-dimethylxanthene (25 mg, 0.0440 mmol, 0.100 eq) and tris(dibenzylidene acetone) dipalladium (0)
(40 mg, 0.0440 mmol, 0.100 eq). The reaction mixture was stirred overnight at 100° C. After completion the reaction mixture was concentrated under reduced pressure and purified by column chromatography (DCM: MeOH 0 to 30%). Fractions containing the product were concentrated under reduced pressure and purified by the reverse phase to yield the title compound (10 mg, 6.4%) as a white solid. MS (ES+) m/z 358.3 (M+H). ¹H NMR (300 MHz, Methanol-d4) δ 8.02 (s, 1H), 7.48 (d, J=2.3 Hz, 1H), 7.07 (d, J=8.4 Hz, 1H), 6.84 (d, J=8.4 Hz, 1H), 6.03 (d, J=2.3 Hz, 1H), 3.84 (s, 3H), 3.82 (s, 3H), 2.20 (s, 3H), 2.12 (s, 3H).

### Biological Methods and Data

### Biological Example 1: PKMYT1 Protein Production

Human PKMYT1 75-362 was cloned as a TEV cleavable 6xHis fusion in bacterial cells. After purification on a Ni++HiTrap Chelating HP column, the His-tag was cleaved using TEV protease. Cleaved PKMYT1 was further purified on a Superdex 200 16/600 sizing column. The fractions containing PKMYT1 were pooled and concentrated using an ultrafiltration centrifugal protein concentrator. The purified protein was aliquoted, flash frozen in liquid nitrogen, and stored at −80° C.

### Biological Example 2: ADP-Glo Enzymatic Assays

PKMYT1 biochemical assay: Inhibition of PKMYT1 was assessed using the ADP-Glo™ Max Detection System from Promega. This assay detects the production of ADP from ATP by PKMYT1 via a two-step process ultimately involving the conversion of ADP to ATP with the latter converted to light in a coupled reaction with luciferase/luciferin. Inhibition of PKMYT1 by small molecule inhibitors results in a retardation of luminescence above background. The intrinsic ATPase activity of PKMYT1 was assessed according to the following protocol. Compounds were dosed into Corning white low volume 384 well assay plates using an Echo acoustic dispenser to generate 10-point curves spanning a 3-fold dilution series. DMSO (no inhibitor) control wells were prepared to benchmark the maximum and minimum signals in the presence and absence of PKMYT1, respectively. The PKMYT1 kinase domain was purified in-house, as described above, and added to the wells at a final concentration of 15 nM in a buffer of 50 mM HEPES (pH 7.5), 10 mM MgCl₂, 1 mM EGTA, 0.01% Brij-35, 1% DMSO. 500 UM ATP was added to start the reaction with the final assay volume being 4 μl. The plate was then incubated for 60 minutes at 26° C. before the addition of 4 μl of ADP-Glo Reagent to stop the reaction and deplete the unconsumed ATP. Following a further 60 minute incubation the assay signal was developed by the addition of 8 μl ADP-Glo Max Detection Reagent. After 60 minutes the luminescence signal was read on a PHERAStar FS microplate reader (BMG Labtech).

Selectivity versus WEE1 assay: Selectivity was established using the same format as outlined for PKMYT1. After dosing of compounds via the Echo acoustic dispenser—as described above—WEE1 (Thermo Fisher PR7373A) was added to each well at a final concentration of 15 nM in a buffer of 50 mM HEPES (pH 7.5), 10 mM MgCl₂, 1 mM EGTA, 0.01% Brij-35, 1% DMSO. 30 PM ATP was added to start the reaction with the final assay volume being 4 μl. The plate was then incubated for 60 minutes at 26° C. before the addition of 4 μl of ADP-Glo Reagent. Following a further 60 minute incubation the assay signal was developed by the addition of 8 μl ADP-Glo Detection Reagent. After 60 minutes the luminescence signal was read on a PHERAStar FS microplate reader (BMG Labtech).

Data analysis: Data was captured and analysed within the Dotmatics Studies software module where dose response curves were generated and IC50 values determined. Acceptance and verification of data followed strict in-house business rules which are summarised as follows. All assay plates should demonstrate Z′ values >0.5 and reference compounds run on each plate should fall within 2-fold of the geometric mean of historical IC50 values. Technical and biological replicates of each compound are routinely tested and replicate IC50 values must fall within 3-fold of each other with dose response curves showing ‘goodness of fit’ r2 values >0.9. Failure to meet any of these defined criteria result in rejection of data.

The data for the primary biochemical assay (PKMYT1 IC50) and selectivity assay (WEE1 IC50) are summarised in the following Biological Data Table 1.

The PKMYT1 IC50 and WEE1 IC50 values are banded as follows:
A: <50 nM; B: 50 nM-<1 μM; C: 1 μM-11 μM; D: >11 μM.

The PKMYT1 vs WEE1 selectivity is banded by fold difference in selectivity for PKMYT1 over WEE1 as follows:
V: 3-<10 fold; W: 10-<100 fold; X: 100-<1000 fold; Y: 1000-5000 fold; Z: >5000 fold.

Where either the PKMYT1 IC50 value was below the lowest concentration limit of the assay or the WEE1 IC50 value was above the top concentration limit of the assay, the PKMYT1 vs WEE1 selectivity is given as “>N”, where N is the selectivity band. The selectivity value for these compounds falls at least in this band but could be higher.

TABLE 1
Biological Data

| | | | PKMYT1 |
|---|---|---|---|
| | PKMYT1 | WEE1 | vs. WEE1 |
| | IC50 | IC50 | selectivity |
| # | (μM) | (μM) | (fold) |
| BAA-001 | B | D | X |
| BAA-002 | A | C | W |
| BAA-003 | C | D | W |
| BAA-004 | B | C | X |
| BAA-005 | A | C | X |
| BAA-011 | A | C | W |
| BAA-012 | B | C | W |
| BAA-013 | A | C | W |
| BAA-014 | A | C | W |
| BAA-015 | A | C | X |
| BAA-016 | A | C | W |
| BAA-017 | B | D | >X |
| BAA-018 | A | D | X |
| BAA-019 | B | D | X |
| BAA-020 | C | nd | nd |
| BAA-021 | B | D | >X |
| BAA-022 | B | C | W |
| BAA-023 | A | C | X |
| BAA-024 | A | C | X |
| BAA-025 | B | D | X |
| BAA-026 | C | nd | nd |
| BAA-027 | B | D | X |
| BAA-028 | A | D | X |
| BAA-029 | B | D | X |
| BAA-030 | C | D | >W |
| BAA-031 | B | D | >X |
| BAA-032 | B | C | W |
| BAA-033 | A | D | X |
| BAA-034 | A | C | X |
| BAA-035 | B | D | X |
| BAA-036 | A | D | Z |
| BAA-037 | B | D | >X |
| BAA-038 | A | C | X |
| BAA-039 | C | nd | nd |
| BAA-040 | B | D | X |
| BAA-041 | B | nd | nd |
| BAA-042 | B | D | >X |
| BAA-006 | B | D | X |
| BAA-007 | A | B | W |
| BAA-008 | B | C | X |
| BAA-009 | A | D | X |
| BAA-010 | B | D | Y |
| BAA-043 | B | D | >Y |
| BAA-044 | B | D | >X |
| BAA-045 | B | nd | nd |
| BAA-046 | B | D | >X |
| BAA-047 | B | D | >X |
| BAA-048 | B | D | Y |
| BAA-049 | C | nd | nd |
| BAA-050 | C | nd | nd |
| BAA-051 | B | D | >X |
| BAA-052 | C | nd | nd |
| BAA-053 | B | C | W |
| BAA-054 | C | D | W |
| BAA-055 | B | D | >X |
| BAA-056 | B | D | X |
| BAA-057 | C | D | >W |
| BAA-058 | A | D | X |
| BAA-059 | A | D | X |
| BAA-060 | B | D | >X |
| BAA-061 | B | D | X |
| BAA-062 | C | D | >W |
| BAA-063 | C | D | >W |
| BAA-064 | C | nd | nd |
| BAA-065 | B | D | >X |
| BAA-066 | B | D | >X |
| BAA-067 | C | D | >W |
| BAA-068 | B | D | >X |
| BAA-069 | C | nd | nd |
| BAA-070 | C | nd | nd |
| BAA-071 | B | D | >X |
| BAA-072 | B | D | >X |
| BAA-073 | B | D | >X |
| BAA-074 | C | nd | nd |
| BAA-075 | C | nd | nd |
| BAA-076 | C | D | >W |
| BAA-077 | B | D | >X |
| BAA-078 | B | D | >X |
| BAA-079 | C | D | >W |
| BAA-080 | B | D | >X |
| BAA-081 | B | D | >X |
| BAA-082 | B | D | >X |
| BAA-083 | B | D | >Y |
| BAA-084 | B | D | >Y |
| BAA-085 | A | D | >Y |
| BAA-086 | B | D | >Y |
| BAA-087 | B | D | >Y |
| BAA-088 | B | D | >X |
| BAA-089 | B | D | >X |
| BAA-090 | B | D | >X |
| BAA-091 | B | D | >X |
| BAA-092 | B | D | >X |
| BAA-093 | B | D | >X |
| BAA-094 | B | D | >X |
| BAA-095 | B | D | >Y |
| BAA-096 | B | D | >Y |
| BAA-097 | B | D | >X |
| BAA-098 | B | D | >X |
| BAA-099 | B | D | >X |
| BAA-100 | B | D | >X |
| BAA-101 | B | D | >X |
| BAA-102 | B | D | >Y |
| BAA-103 | B | D | >X |
| BAA-104 | B | D | >X |
| BAA-105 | B | D | >Y |
| BAA-106 | A | D | >Y |
| BAA-107 | B | D | >X |
| BAA-108 | A | D | >Y |
| BAA-109 | B | D | >X |
| BAA-110 | B | D | >X |
| BAA-111 | B | D | >Y |
| BAA-112 | B | D | >Y |
| BAA-113 | B | D | >X |
| BAA-114 | B | D | >X |
| BAA-115 | B | D | >X |
| BAA-116 | B | C | W |
| BAA-117 | A | D | Y |
| BAA-118 | A | D | X |
| BAA-119 | B | D | X |
| BAA-120 | B | C | X |
| BAA-121 | B | D | X |
| BAA-122 | B | C | X |
| BAA-123 | A | C | X |
| BAA-124 | A | D | X |
| BAA-125 | A | D | Y |
| BAA-126 | A | C | X |
| BAA-127 | A | D | Y |
| BAA-128 | B | C | X |
| BAA-129 | B | D | >Y |
| BAA-130 | A | C | X |
| BAA-131 | A | D | Y |
| BAA-132 | A | D | Y |
| BAA-133 | A | C | X |
| BAA-134 | A | D | Y |
| BAA-135 | A | C | X |
| BAA-136 | A | D | Y |
| BAA-137 | A | D | Y |
| BAA-138 | B | D | X |
| BAA-139 | A | D | Y |
| BAA-140 | A | D | Y |
| BAA-141 | A | D | X |
| BAA-142 | A | D | X |
| BAA-143 | A | D | Y |
| BAA-144 | A | C | X |
| BAA-145 | A | D | Y |
| BAA-146 | A | D | Y |
| BAA-147 | A | D | Y |
| BAA-148 | A | D | Y |
| BAA-149 | A | D | Y |
| BAA-150 | A | D | X |
| BAA-151 | B | D | X |
| BAA-152 | A | C | X |
| BAA-153 | A | D | Y |
| BAA-154 | A | D | Y |
| BAA-155 | A | D | X |
| BAA-156 | A | D | Y |
| BAA-157 | B | D | X |
| BAA-158 | B | D | X |
| BAA-159 | A | D | Y |
| BAA-160 | B | D | X |
| BAA-161 | A | D | Y |
| BAA-162 | A | D | Y |
| BAA-163 | A | D | Y |
| BAA-164 | A | D | Y |
| BAA-165 | A | D | Y |
| BAA-166 | A | D | Y |
| BAA-167 | B | D | X |
| BAA-168 | B | D | W |
| BAA-169 | A | D | Y |
| BAA-170 | A | D | Y |
| BAA-171 | A | D | X |
| BAA-172 | A | D | X |
| BAA-173 | A | D | Y |
| BAA-174 | B | nd | nd |
| BAA-175 | A | D | Y |
| BAA-176 | A | D | Y |
| BAA-177 | A | D | X |
| BAA-178 | B | D | X |
| BAA-179 | A | C | X |
| BAA-180 | A | D | Y |
| BAA-181 | A | D | Y |
| BAA-182 | A | D | Y |
| BAA-183 | A | D | Y |
| BAA-184 | B | D | >X |
| BAA-185 | B | D | >Y |
| BAA-186 | A | D | Y |
| BAA-187 | A | D | Y |
| BAA-188 | A | D | Y |
| BAA-189 | B | D | Y |
| BAA-190 | A | D | Y |
| BAA-191 | A | D | Y |
| BAA-192 | A | D | Y |
| BAA-193 | A | D | Y |
| BAA-194 | A | D | Y |
| BAA-195 | A | D | Y |
| BAA-196 | A | D | Y |
| BAA-197 | A | D | Y |
| BAA-198 | A | D | Y |
| BAA-199 | A | C | Y |
| BAA-200 | A | D | X |
| BAA-201 | A | D | X |
| BAA-202 | A | D | X |
| BAA-203 | A | D | Y |
| BAA-204 | A | D | Z |
| BAA-205 | A | D | Y |
| BAA-206 | A | C | Y |
| BAA-207 | A | C | X |
| BAA-208 | A | C | X |
| BAA-209 | A | D | X |
| BAA-210 | A | D | Z |
| BAA-211 | C | nd | nd |
| BAA-212 | B | D | >X |
| BAA-213 | B | D | >X |
| BAA-214 | B | D | >X |
| BAA-215 | B | D | >X |
| BAA-216 | C | nd | nd |
| BAA-217 | B | nd | nd |
| BAA-218 | C | D | W |
| BAA-219 | C | C | V |
| BAA-220 | A | B | W |
| BAA-221 | B | D | X |
| BAA-222 | A | D | X |
| BAA-223 | A | D | X |
| BAA-224 | A | D | X |
| BAA-225 | A | D | X |
| BAA-226 | A | D | X |
| BAA-227 | A | D | X |
| BAA-228 | A | D | X |
| BAA-229 | A | D | Y |
| BAA-230 | A | D | X |
| BAA-231 | B | D | Y |
| BAA-232 | A | C | W |
| BAA-233 | A | B | W |
| BAA-234 | A | C | X |
| BAA-235 | A | D | Y |
| BAA-236 | B | D | W |
| BAA-237 | A | C | W |
| BAA-238 | A | D | Y |
| BAA-239 | B | C | W |
| BAA-240 | B | D | X |
| BAA-241 | B | D | X |
| BAA-242 | B | D | Y |
| BAA-243 | A | C | X |
| BAA-244 | A | C | X |
| BAA-245 | A | D | Y |
| BAA-246 | C | D | W |
| BAA-247 | B | D | Y |
| BAA-248 | A | C | X |
| BAA-249 | B | D | X |
| BAA-250 | A | D | X |
| BAA-251 | A | C | X |
| BAA-252 | B | C | X |
| BAA-253 | B | D | X |
| BAA-254 | B | D | Y |
| BAA-255 | B | D | X |
| BAA-256 | A | C | W |
| BAA-257 | A | D | Y |
| BAA-258 | C | D | W |
| BAA-259 | B | D | X |
| BAA-260 | B | D | W |
| BAA-261 | B | D | X |
| BAA-262 | A | D | X |
| BAA-263 | B | D | X |
| BAA-264 | A | D | X |
| BAA-265 | A | D | X |
| BAA-266 | B | D | X |
| BAA-267 | B | D | X |
| BAA-268 | A | D | Y |
| BAA-269 | A | B | W |
| BAA-270 | A | C | X |
| BAA-271 | A | D | X |
| BAA-272 | A | B | W |
| BAA-273 | A | B | W |
| BAA-274 | A | C | W |
| BAA-275 | A | D | X |
| BAA-276 | A | D | Y |
| BAA-277 | B | D | X |
| BAA-278 | C | D | W |
| BAA-279 | A | D | Y |
| BAA-280 | B | D | Y |
| BAA-281 | B | D | X |
| BAA-282 | C | D | W |
| BAA-283 | C | D | W |
| BAA-284 | C | D | W |
| BAA-285 | C | D | W |
| BAA-286 | C | D | W |
| BAA-287 | A | D | Y |
| BAA-288 | B | D | X |
| BAA-289 | A | C | X |
| BAA-290 | B | D | X |
| BAA-291 | C | | |
| BAA-292 | C | | |
| BAA-293 | A | D | Y |
| BAA-294 | B | D | X |
| BAA-295 | B | D | Y |
| BAA-296 | A | D | Y |
| BAA-297 | B | D | Y |
| BAA-298 | B | D | Y |
| BAA-299 | A | C | X |
| BAA-300 | B | D | X |
| BAA-301 | B | D | X |
| BAA-302 | B | D | X |
| BAA-303 | A | D | Y |
| BAA-304 | B | D | X |
| BAA-305 | C | D | W |
| BAA-306 | B | D | X |
| BAA-307 | B | D | Y |
| BAA-308 | B | D | X |
| BAA-309 | B | D | Y |
| BAA-310 | B | D | X |
| BAA-311 | C | | |
| BAA-312 | C | | |
| BAA-313 | B | D | X |
| BAA-314 | B | C | W |
| BAA-315 | A | D | Y |
| BAA-316 | B | D | Y |
| BAA-317 | A | D | Y |
| BAA-318 | B | D | Y |
| BAA-319 | A | D | Y |
| BAA-320 | A | D | Y |
| BAA-321 | B | D | X |
| BAA-322 | B | D | X |
| BAA-323 | A | D | Y |
| BAA-324 | B | D | X |
| BAA-325 | B | D | Y |
| BAA-326 | B | D | X |
| BAA-327 | B | D | X |
| BAA-328 | B | D | X |
| BAA-329 | B | D | Y |
| BAA-330 | A | D | Y |
| BAA-331 | A | D | Y |
| BAA-332 | A | D | X |
| BAA-333 | B | D | Y |
| BAA-334 | B | D | X |
| BAA-335 | A | D | Y |
| BAA-336 | B | D | X |
| BAA-337 | A | C | X |
| BAA-338 | A | C | X |
| BAA-339 | A | D | Y |
| BAA-340 | A | B | V |
| BAA-341 | A | C | X |
| BAA-342 | A | D | X |
| BAA-343 | B | C | W |
| BAA-344 | B | D | Y |
| BAA-345 | B | D | Y |
| BAA-346 | A | D | Y |
| BAA-347 | B | D | X |
| BAA-348 | B | D | X |
| BAA-349 | B | D | X |
| BAA-350 | B | D | Y |
| BAA-351 | C | D | W |
| BAA-352 | B | D | X |
| BAA-353 | C | D | W |
| BAA-354 | C | D | W |
| BAA-355 | B | D | X |
| BAA-356 | C | D | W |
| BAA-357 | B | D | X |
| BAA-358 | B | D | X |
| BAA-359 | C | D | W |
| BAA-360 | A | D | Y |
| BAA-361 | A | D | X |
| BAA-362 | C | D | W |
| BAA-363 | B | D | X |
| BAA-364 | C | D | W |
| BAA-365 | A | D | X |
| BAA-366 | A | C | X |
| BAA-367 | B | D | X |
| BAA-368 | A | D | Y |
| BAA-369 | B | D | X |
| BAA-370 | A | D | X |
| BAA-371 | A | C | X |
| BAA-372 | A | B | W |
| BAA-373 | B | D | X |
| BAA-374 | A | C | X |
| BAA-375 | C | D | W |
| BAA-376 | B | D | X |
| BAA-377 | B | C | W |
| BAA-378 | B | C | W |
| BAA-379 | A | D | X |
| BAA-380 | B | C | W |
| BAA-381 | A | C | X |
| BAA-382 | B | D | X |
| BAA-383 | A | D | Y |
| BAA-384 | A | C | X |
| BAA-385 | A | C | X |
| BAA-386 | A | C | X |
| BAA-387 | A | C | X |
| BAA-388 | A | C | X |
| BAA-389 | A | D | Y |
| BAA-390 | A | C | X |
| BAA-391 | A | C | X |
| BAA-392 | A | C | X |
| BAA-393 | B | D | X |
| BAA-394 | A | C | X |
| BAA-395 | A | C | X |
| BAA-396 | A | C | X |
| BAA-397 | A | C | W |
| BAA-398 | A | C | X |
| BAA-399 | A | C | X |
| BAA-400 | A | C | X |
| BAA-401 | A | C | W |
| BAA-402 | A | B | W |
| BAA-403 | A | B | W |
| BAA-404 | A | D | Y |
| BAA-405 | B | D | X |
| BAA-406 | A | C | X |
| BAA-407 | A | C | X |
| BAA-408 | B | D | X |
| BAA-409 | A | C | X |
| BAA-410 | A | C | X |
| BAA-411 | A | D | Y |
| BAA-412 | A | D | Z |
| BAA-413 | A | D | Z |
| BAA-414 | A | C | X |
| BAA-415 | A | D | X |
| BAA-416 | A | C | X |
| BAA-417 | A | D | X |
| BAA-418 | A | C | X |
| BAA-419 | A | D | Y |
| BAA-420 | A | D | Y |
| BAA-421 | A | D | X |
| BAA-422 | A | C | X |
| BAA-423 | A | C | W |
| BAA-424 | A | D | Y |
| BAA-425 | A | D | Y |
| BAA-426 | A | D | Z |
| BAA-427 | A | D | Z |
| BAA-428 | A | | |
| BAA-429 | A | D | X |
| BAA-430 | A | D | Y |
| BAA-431 | B | D | X |
| BAA-432 | A | C | X |
| BAA-433 | A | | |
| BAA-434 | A | D | Y |
| BAA-435 | A | D | Y |
| BAA-436 | A | D | Z |
| BAA-437 | C | D | W |
| BAA-438 | C | D | W |
| BAA-439 | A | C | X |
| BAA-440 | A | C | X |
| BAA-441 | A | C | W |
| BAA-442 | B | D | X |
| BAA-443 | B | D | X |
| BAA-444 | A | D | X |
| BAA-445 | B | C | X |
| BAA-446 | B | D | X |
| BAA-447 | A | C | X |
| BAA-448 | A | D | Y |
| BAA-449 | B | D | W |
| BAA-450 | A | C | X |
| BAA-451 | A | C | X |
| BAA-452 | B | D | X |
| BAA-453 | B | D | X |
| BAA-454 | B | D | X |
| BAA-455 | B | D | X |
| BAA-456 | B | D | Y |
| BAA-457 | A | C | X |
| BAA-458 | A | C | X |
| BAA-459 | B | D | X |
| BAA-460 | B | C | X |
| BAA-461 | A | C | X |
| BAA-462 | B | D | X |
| BAA-463 | B | D | X |
| BAA-464 | A | D | Y |
| BAA-465 | A | D | Y |
| BAA-466 | B | D | Y |
| BAA-467 | A | B | W |
| BAA-468 | A | C | X |
| BAA-469 | B | D | X |
| BAA-470 | A | C | X |
| BAA-471 | B | D | X |
| BAA-472 | A | D | X |
| BAA-473 | A | C | X |
| BAA-474 | B | D | X |
| BAA-475 | A | D | Y |
| BAA-476 | A | C | X |
| BAA-477 | A | C | X |
| BAA-478 | A | C | X |
| BAA-479 | B | D | W |
| BAA-480 | B | D | X |
| BAA-481 | A | D | Y |
| BAA-482 | A | D | Y |
| BAA-483 | B | D | X |
| BAA-484 | B | D | X |
| BAA-485 | A | D | X |
| BAA-486 | B | D | X |
| BAA-487 | B | D | X |
| BAA-488 | A | C | X |
| BAA-489 | A | C | X |
| BAA-490 | A | C | X |
| BAA-491 | A | C | X |
| BAA-492 | A | C | X |
| BAA-493 | A | C | X |
| BAA-494 | B | D | X |
| BAA-495 | B | C | W |
| BAA-496 | A | D | Y |
| BAA-497 | A | C | X |
| BAA-498 | B | C | W |
| BAA-499 | A | C | X |
| BAA-500 | A | D | X |
| BAA-501 | B | D | W |
| BAA-502 | A | C | X |
| BAA-503 | B | C | W |
| BAA-504 | A | C | X |
| BAA-505 | B | C | W |
| BAA-506 | A | C | X |
| BAA-507 | A | D | X |
| BAA-508 | B | D | W |
| BAA-509 | B | D | X |
| BAA-510 | A | C | X |
| BAA-511 | B | D | W |
| BAA-512 | A | D | Y |
| BAA-513 | A | C | X |
| BAA-514 | A | C | X |
| BAA-515 | A | C | X |
| BAA-516 | B | D | X |
| BAA-517 | C | | |
| BAA-518 | A | C | W |
| BAA-519 | A | D | X |
| BAA-520 | B | D | X |
| BAA-521 | B | D | X |
| BAA-522 | A | C | X |
| BAA-523 | A | C | X |
| BAA-524 | A | D | Y |
| BAA-525 | C | | |
| BAA-526 | B | D | X |
| BAA-527 | B | D | W |
| BAA-528 | B | D | X |
| BAA-529 | B | D | X |
| BAA-530 | B | D | X |
| BAA-531 | B | D | Y |
| BAA-532 | A | C | X |
| BAA-533 | A | D | Y |
| BAA-534 | A | D | Y |
| BAA-535 | A | C | X |
| BAA-536 | B | C | X |
| BAA-537 | B | C | W |
| BAA-538 | A | C | X |
| BAA-539 | B | D | X |
| BAA-540 | A | C | X |
| BAA-541 | A | C | X |
| BAA-542 | A | D | X |
| BAA-543 | A | C | W |
| BAA-544 | B | C | W |
| BAA-545 | B | C | W |
| BAA-546 | A | C | X |
| BAA-547 | C | | |
| BAA-548 | B | D | X |
| BAA-549 | A | D | X |
| BAA-550 | A | C | X |
| BAA-551 | A | C | X |
| BAA-552 | B | D | X |
| BAA-553 | B | D | X |
| BAA-554 | A | C | X |
| BAA-555 | A | C | Y |
| BAA-556 | B | D | X |
| BAA-557 | C | | |
| BAA-558 | B | D | Y |
| BAA-559 | A | C | X |
| BAA-560 | A | C | X |
| BAA-561 | C | | |
| BAA-562 | A | D | Y |
| BAA-563 | B | C | W |
| BAA-564 | B | C | W |
| BAA-565 | A | C | X |
| BAA-566 | A | C | X |
| BAA-567 | A | D | Y |
| BAA-568 | A | D | X |
| BAA-569 | A | C | X |
| BAA-570 | A | D | X |
| BAA-571 | A | B | W |
| BAA-572 | A | B | W |
| BAA-573 | A | D | Y |
| BAA-574 | A | C | W |
| BAA-575 | A | B | W |
| BAA-576 | A | C | X |
| BAA-577 | A | D | X |
| BAA-578 | A | C | X |
| BAA-579 | A | C | X |
| BAA-580 | A | C | X |
| BAA-581 | A | B | W |
| BAA-582 | A | C | X |
| BAA-583 | A | C | X |
| BAA-584 | A | B | W |
| BAA-585 | A | C | X |
| BAA-586 | A | B | W |
| BAA-587 | A | C | X |
| BAA-588 | A | C | X |
| BAA-589 | A | C | X |
| BAA-590 | A | B | W |
| BAA-591 | A | C | X |
| BAA-592 | A | C | X |
| BAA-593 | A | C | X |
| BAA-594 | A | C | X |
| BAA-595 | A | C | X |
| BAA-596 | A | D | Y |
| BAA-597 | A | D | X |
| BAA-598 | A | C | X |
| BAA-599 | A | C | X |
| BAA-600 | A | C | X |
| BAA-601 | A | D | X |
| BAA-602 | B | D | X |
| BAA-603 | B | D | X |
| BAA-604 | A | D | Y |
| BAA-605 | A | C | X |
| BAA-606 | A | C | X |
| BAA-607 | A | C | X |
| BAA-608 | B | D | X |

### Biological Example 3: Nuclear Count Cell Proliferation Assay of Isogenic h-TERT-RPE1 TP53−/−Parental and CCNE1 Overexpressing Cell Lines

h-TERT-RPE1 TP53−/− and CCNE1 high expressing clones (clone cell lines 7 and 15) were treated with test compounds dissolved in DMSO at serially diluted concentrations alongside a DMSO only control for around 6 population doublings time. Cells were then fixed with 4% Paraformaldehyde and the cell nuclei were stained with DAPI (Sigma D9542). Images covering each well were acquired by an Opera Phenix® High-Content Screening System (PerkinElmer). Image analysis was performed using Harmony® high-content analysis software (PerkinElmer). Data analysis was performed using Microsoft Excel® and GI50 estimates generated with GraphPad Prism v9. Percent nuclear count was compared to vehicle control wells and used to measure inhibition of proliferation.

The compounds in Biological Data Table 2 show an increase in cell proliferation inhibition of at least 3-fold in a CCNE1 overexpressing clone compared to parent cell line expressing endogenous CCNE1 levels. These PKMYT1 inhibitors show CCNE1 amplification dependent increased activity in isogenic cell line pairs.

The RPE cell line GI50 values are banded as follows:
A: <0.5 MM; B: 0.5 M-<3 μM; C: 3 μM-10 μM; D: >10 μM.

The RPE wild type (Wt) to clone cell line (CL7 or CL15) GI50 selectivity ratio is banded by fold difference as follows:
X: 3-<10 fold; Y: 10-30 fold; Z: >30 fold.

Where either the RPE CL7 G150 value was below the lowest concentration limit of the assay or the RPE Wt GI50 value was above the top concentration limit of the assay, the selectivity ratio wild type vs. CL7 is given as “>N”, where N is the selectivity band. The selectivity value for these compounds falls at least in this band but could be higher.

Likewise, where either the RPE CL15 GI50 value was below the lowest concentration limit of the assay or the RPE Wt GI50 value was above the top concentration limit of the assay, the selectivity ratio wild type vs. CL15 is given as “>N”, where N is the selectivity band. The selectivity value for these compounds falls at least in this band but could be higher.

Biological Data Table 2

| | RPE CL7 | RPE CL15 | RPE Wt | Selectivity | Selectivity |
|---|---|---|---|---|---|
| | GI50 | GI50 | GI50 | Ratio Wt | Ratio Wt |
| # | (μM) | (μM) | (μM) | vs. CL7 | vs. CL15 |
| BAA-061 | | B | C | | X |
| BAA-071 | B | | D | X | |
| BAA-113 | B | | C | Y | |
| BAA-129 | | B | D | | X |
| BAA-137 | B | | B | X | |
| BAA-148 | | B | B | | X |
| BAA-152 | | B | D | | X |
| BAA-160 | B | | C | X | |
| BAA-173 | A | | B | X | |
| BAA-179 | | A | C | | Y |
| BAA-181 | A | A | B | X | Y |
| BAA-183 | A | | C | X | |
| BAA-190 | B | | C | X | |
| BAA-209 | A | | B | >X | |
| BAA-224 | A | | C | Z | |
| BAA-229 | C | | D | X | |
| BAA-230 | C | | D | X | |

### Biological Example 4: Cancer Cell Lines Proliferation Assays

Cancer cell lines with either normal (KYSE-30) or amplified CCNE1 expression (OVCAR3-ovarian cancer) were evaluated for their sensitivity to PKMYT1 inhibitors in a nuclear count assay as described above for h-TERT-RPE1 TP53−/− and a CCNE1 high expressing clones, taking into account the slower cell cycle division times of these lines.

The following compounds in Biological Data Table 3 inhibit OVCAR3 cell proliferation with a GI50<5 μM. In addition, selected compounds exhibit >3-fold selectivity for the CCNE1-amplified OVCAR3 cells compared to KYSE30 cells expressing normal levels of CCNE1. (Cell proliferation inhibition in cancer cell lines: OVCAR3 expresses high level of CCNE1; control cell line KYSE30 expresses normal levels of CCNE1.)

The cell line GI50 values are banded as follows:
A: <0.5 μM; B: 0.5 μM-<3 μM; C: 3 μM-10 μM; D: >10 μM.

The normal CCNE1 expression cell line (KYSE-30) to CCNE1-amplified cell line (OVCAR3) GI50 selectivity ratio is banded by fold difference as follows:
X: 3-<10 fold; Y: 10-30 fold; Z: >30 fold.

Where either the OVCAR3 G150 value was below the lowest concentration limit of the assay or the KYSE30 G150 value was above the top concentration limit of the assay, the selectivity ratio KYSE30 GI50 vs. OVCAR3 G150 is given as “>N”, where N is the selectivity band. The selectivity value for these compounds falls at least in this band but could be higher.

Biological Data Table 3

| | OVCAR3 | KYSE30 | Selectivity ratio |
|---|---|---|---|
| | GI50 | GI50 | KYSE30 GI50 vs. |
| # | (μM) | (μM) | OVCAR3 GI50 |
| BAA-046 | B | C | X |
| BAA-047 | B | C | X |
| BAA-148 | C | D | X |
| BAA-173 | C | D | >X |
| BAA-179 | A | D | Z |
| BAA-181 | C | D | >X |
| BAA-206 | B | D | >Y |
| BAA-207 | A | C | Z |
| BAA-208 | A | D | Z |

### Biological Example 5: Cancer Cell Lines Proliferation Assays—ATP-Based Cell Viability Endpoint

Cancer cell lines with either normal (KYSE-30) or amplified CCNE1 expression (OVCAR 3 —ovarian cancer) were evaluated for their sensitivity to PKMYT1 inhibitors using Cell Titre Glow assay (Promega G7573) as endpoint to assess cell proliferation. The luminescence signal was read on a PHERAStar FS microplate reader (BMG Labtech). Data analysis was performed using Microsoft Excel® and Gl₅₀ (concentration that achieves 50% cell growth proliferation inhibition) estimates generated with GraphPad Prism v9.

The following compounds in Biological Data Table 2 inhibit OVCAR 3 cell proliferation with a Gl₅₀<0.5 μM. In addition, selected compounds exhibit >3-fold selectivity for the CCNE1-amplified OVCAR 3 cells compared to KYSE30 cells expressing normal levels of CCNE1. (Cell proliferation inhibition in cancer cell lines: OVCAR3 expresses high level of CCNE1; control cell line KYSE30 expresses normal levels of CCNE1.)

The cell line Gl₅₀ values are banded as follows:
A: <0.5 MM; B: 0.5 MM-<5 UM; C: >5 μM.

The normal CCNE1 expression cell line (KYSE-30) to CCNE1-amplified cell line (OVCAR 3) GI 50 selectivity ratio is banded by fold difference as follows:
X: 3-20 fold; Y: >20 fold.

Where either the OVCAR 3 Gl₅₀ value was below the lowest concentration limit of the assay or the KYSE30 G150 value was above the top concentration limit of the assay, the selectivity ratio KYSE30 G150 vs. OVCAR 3 G150 is given as “>N”, where N is the selectivity band. The selectivity value for these compounds falls at least in this band but could be higher.

TABLE 4
Biological Data

| | | | Selectivity |
|---|---|---|---|
| | | | ratio |
| | | | KYSE30 |
| | OVCAR 3 | KYSE30 | Gl₅₀ vs. |
| | Gl₅₀ | Gl₅₀ | OVCAR3 |
| # | (μM) | (μM) | Gl₅₀ |
| BAA-013 | B | C | X |
| BAA-016 | A | B | X |
| BAA-038 | A | C | Y |
| BAA-123 | A | C | Y |
| BAA-126 | A | C | Y |
| BAA-128 | A | C | Y |
| BAA-207 | A | C | Y |
| BAA-208 | A | C | Y |
| BAA-220 | A | C | Y |
| BAA-232 | A | C | Y |
| BAA-233 | A | C | Y |
| BAA-237 | A | B | Y |
| BAA-243 | A | C | X |
| BAA-268 | A | C | Y |
| BAA-269 | A | B | Y |
| BAA-270 | A | A | Y |
| BAA-271 | A | C | Y |
| BAA-274 | A | B | Y |
| BAA-294 | A | C | Y |
| BAA-303 | A | B | X |
| BAA-306 | A | B | X |
| BAA-307 | A | B | Y |
| BAA-308 | A | B | X |
| BAA-309 | A | B | Y |
| BAA-313 | A | B | Y |
| BAA-314 | A | B | Y |
| BAA-315 | A | B | Y |
| BAA-316 | A | B | Y |
| BAA-317 | A | B | Y |
| BAA-319 | A | B | Y |
| BAA-320 | A | B | Y |
| BAA-321 | A | C | Y |
| BAA-322 | A | B | Y |
| BAA-323 | A | B | Y |
| BAA-325 | A | C | X |
| BAA-326 | A | B | Y |
| BAA-327 | A | B | Y |
| BAA-328 | A | C | Y |
| BAA-329 | A | B | X |
| BAA-330 | A | B | Y |
| BAA-331 | A | B | Y |
| BAA-332 | A | B | Y |
| BAA-333 | A | C | Y |
| BAA-334 | A | B | Y |
| BAA-335 | A | B | Y |
| BAA-336 | A | C | Y |
| BAA-337 | A | B | Y |
| BAA-338 | A | A | Y |
| BAA-339 | A | B | Y |
| BAA-340 | A | B | Y |
| BAA-341 | A | B | X |
| BAA-342 | A | C | Y |
| BAA-361 | A | B | Y |
| BAA-365 | A | B | Y |
| BAA-367 | A | C | Y |
| BAA-368 | A | C | Y |
| BAA-369 | A | C | Y |
| BAA-370 | A | B | X |
| BAA-371 | A | C | Y |
| BAA-372 | A | C | Y |
| BAA-376 | A | B | Y |
| BAA-377 | A | B | X |
| BAA-378 | A | B | X |
| BAA-379 | A | C | X |
| BAA-381 | A | C | Y |
| BAA-383 | A | B | X |
| BAA-384 | A | B | Y |
| BAA-385 | A | B | Y |
| BAA-386 | A | C | Y |
| BAA-390 | A | B | Y |
| BAA-391 | A | B | Y |
| BAA-392 | A | C | Y |
| BAA-393 | A | B | X |
| BAA-394 | A | B | Y |
| BAA-396 | A | B | Y |
| BAA-397 | A | B | Y |
| BAA-398 | A | B | Y |
| BAA-399 | A | B | Y |
| BAA-401 | A | B | Y |
| BAA-402 | A | B | Y |
| BAA-403 | A | C | Y |
| BAA-407 | A | C | Y |
| BAA-409 | A | B | Y |
| BAA-410 | A | B | Y |
| BAA-414 | A | B | Y |
| BAA-416 | A | B | Y |
| BAA-417 | A | B | Y |
| BAA-418 | A | B | Y |
| BAA-419 | A | C | Y |
| BAA-420 | A | B | Y |
| BAA-421 | A | B | Y |
| BAA-422 | A | B | Y |
| BAA-423 | A | B | Y |
| BAA-424 | A | C | Y |
| BAA-425 | A | B | Y |
| BAA-426 | A | B | Y |
| BAA-427 | A | C | Y |
| BAA-428 | A | C | Y |
| BAA-429 | A | A | Y |
| BAA-430 | A | C | Y |
| BAA-432 | A | C | Y |
| BAA-441 | A | C | Y |
| BAA-447 | A | C | Y |
| BAA-450 | A | C | X |
| BAA-451 | A | C | Y |
| BAA-453 | A | C | Y |
| BAA-456 | A | B | X |
| BAA-457 | A | B | Y |
| BAA-458 | A | B | Y |
| BAA-459 | A | C | Y |
| BAA-460 | A | B | Y |
| BAA-470 | A | C | Y |
| BAA-477 | A | C | Y |
| BAA-478 | A | C | Y |
| BAA-488 | A | C | Y |
| BAA-489 | A | C | Y |
| BAA-490 | A | C | Y |
| BAA-491 | A | C | Y |
| BAA-492 | A | C | Y |
| BAA-493 | A | C | Y |
| BAA-495 | A | C | Y |
| BAA-497 | A | C | Y |
| BAA-499 | A | C | Y |
| BAA-502 | A | C | Y |
| BAA-504 | A | C | Y |
| BAA-506 | A | C | Y |
| BAA-509 | A | C | Y |
| BAA-510 | A | C | Y |
| BAA-512 | A | C | Y |
| BAA-541 | A | C | Y |
| BAA-543 | A | C | Y |
| BAA-546 | A | C | Y |
| BAA-550 | A | C | Y |
| BAA-551 | A | B | Y |
| BAA-554 | A | C | Y |
| BAA-557 | A | C | Y |
| BAA-560 | A | B | Y |
| BAA-565 | A | C | Y |
| BAA-566 | A | C | Y |
| BAA-569 | A | B | Y |
| BAA-571 | A | B | Y |
| BAA-572 | A | B | Y |
| BAA-574 | A | B | X |
| BAA-578 | A | C | Y |
| BAA-581 | A | C | Y |
| BAA-582 | A | B | Y |
| BAA-583 | A | C | Y |
| BAA-584 | A | C | Y |
| BAA-585 | A | B | Y |
| BAA-586 | A | B | X |
| BAA-587 | A | B | Y |
| BAA-588 | A | B | Y |
| BAA-589 | A | C | Y |
| BAA-590 | A | C | Y |
| BAA-591 | A | C | Y |
| BAA-592 | A | C | Y |
| BAA-594 | A | C | Y |
| BAA-595 | A | C | Y |
| BAA-596 | A | B | Y |
| BAA-597 | A | B | Y |
| BAA-598 | A | B | Y |
| BAA-599 | A | C | Y |
| BAA-600 | A | B | Y |
| BAA-605 | A | C | Y |
| BAA-606 | A | B | Y |
| BAA-607 | A | C | Y |

### Biological Example 6: Inhibition of Cellular PKMYT1 Biomarker CDK1 pT14

To determine compound PKMYT1 IC50 in a cellular assay, OVCAR 3 cells were incubated at 37° C. in 5% CO₂ atmosphere. The cells were maintained in RPMI medium supplemented with 20% foetal bovine serum, 1X non essential amino acids, 1 mM sodium pyruvate, Glutamax and insulin. Cells were seeded into 12-well plates at low density (50,000-200,000 cells per well), grown to confluence (~350-400,000 cells per well) and then treated with a range of concentrations of PKMYT1 inhibitors. The cells were incubated 20 hours and then harvested into 1X Laemmli Sample Buffer (BIORAD) without 2-mercaptoethanol, heated to 95° C. for 5 min, centrifuged and sonicated using a microprobe for 15 sec, 21W to shear the DNA.

The samples were analysed by immunoblot on a Jess Automated Western Blot System (BioTechne). The samples were separated using the 12-230 kDa Separation Module (BioTechne,) and blotted using an anti-CDK1 phospho T14 antibody (Abcam, #ab58509) and an anti-vinculin antibody (Abcam, #126002). Specific binding was detected by fluorescence using the Anti-Rabbit Detection Module (BioTechne, #DM-001), quantified using Compass for Simple Western software, and IC50 values were calculated by regression analysis in GraphPad Prism.

The following compounds in Biological Data Table 5 inhibit CDK1 pT14 in OVCAR 3 cells with an IC₅₀<1 μM. The CDK1 pT14 IC50 values are banded as follows:
A: <100 nM; B: 100 nM-1 μM;

| # | CDK1 pT14 IC₅₀ (μM) |
|---|---|
| BAA-047 | B |
| BAA-126 | A |
| BAA-207 | A |
| BAA-220 | B |
| BAA-231 | B |
| BAA-243 | A |
| BAA-268 | A |
| BAA-269 | A |
| BAA-270 | A |
| BAA-271 | A |
| BAA-303 | A |
| BAA-308 | A |
| BAA-309 | A |
| BAA-313 | A |
| BAA-314 | A |
| BAA-316 | B |
| BAA-317 | A |
| BAA-319 | A |
| BAA-320 | A |
| BAA-321 | B |
| BAA-323 | A |
| BAA-325 | A |
| BAA-326 | A |
| BAA-327 | B |
| BAA-328 | A |
| BAA-331 | A |
| BAA-332 | A |
| BAA-333 | A |
| BAA-334 | A |
| BAA-337 | A |
| BAA-338 | A |
| BAA-340 | B |
| BAA-342 | A |
| BAA-361 | A |
| BAA-365 | A |
| BAA-379 | A |
| BAA-381 | A |
| BAA-384 | A |
| BAA-385 | A |
| BAA-390 | A |
| BAA-391 | A |
| BAA-394 | A |
| BAA-396 | A |
| BAA-397 | A |
| BAA-398 | A |
| BAA-401 | A |
| BAA-402 | A |
| BAA-406 | A |
| BAA-407 | A |
| BAA-409 | A |
| BAA-410 | A |
| BAA-411 | B |
| BAA-412 | B |
| BAA-413 | B |
| BAA-414 | A |
| BAA-415 | A |
| BAA-417 | B |
| BAA-418 | A |
| BAA-419 | B |
| BAA-420 | A |
| BAA-421 | A |
| BAA-422 | A |
| BAA-423 | A |
| BAA-424 | A |
| BAA-425 | A |
| BAA-426 | A |
| BAA-427 | A |
| BAA-428 | A |
| BAA-430 | A |
| BAA-432 | A |
| BAA-441 | A |
| BAA-451 | A |
| BAA-453 | A |
| BAA-454 | A |
| BAA-456 | A |
| BAA-458 | A |
| BAA-459 | A |
| BAA-464 | B |
| BAA-467 | A |
| BAA-470 | A |
| BAA-471 | B |
| BAA-473 | B |
| BAA-478 | A |
| BAA-488 | A |
| BAA-493 | A |
| BAA-504 | B |
| BAA-510 | A |
| BAA-523 | B |
| BAA-543 | A |
| BAA-546 | A |
| BAA-549 | A |
| BAA-550 | A |
| BAA-551 | A |
| BAA-553 | B |
| BAA-554 | A |
| BAA-560 | A |
| BAA-565 | A |
| BAA-566 | A |
| BAA-569 | A |
| BAA-571 | A |
| BAA-572 | A |
| BAA-576 | A |
| BAA-577 | A |
| BAA-578 | A |
| BAA-580 | B |
| BAA-581 | A |
| BAA-582 | A |
| BAA-583 | A |
| BAA-584 | B |
| BAA-585 | A |
| BAA-586 | A |
| BAA-587 | A |
| BAA-588 | A |
| BAA-589 | A |
| BAA-590 | A |
| BAA-591 | A |
| BAA-592 | A |
| BAA-593 | B |
| BAA-594 | A |
| BAA-595 | A |
| BAA-596 | B |
| BAA-597 | B |
| BAA-598 | A |
| BAA-599 | B |
| BAA-600 | A |
| BAA-601 | B |
| BAA-604 | B |
| BAA-605 | A |
| BAA-607 | A |

### Biological Example 7: Comparison Data 1

IC50 was measured for BAA-001, REF-001 and REF-002 as described for PKMYT 1 biochemical ADP-Glo enzymatic assay in Biological Example 2. See Biological Data Table 4, below.

As compared to REF-001, BAA-001 exhibits a 163-fold improvement in PKMYT1 IC50.

As compared to REF-002, BAA-001 exhibits an 82-fold improvement in PKMYT1 IC50.

The enhancement in activity seen when the phenol ring is substituted at both positions ortho to the amide group indicates that this ortho-disubstituted pattern confers surprising and unexpected improvements in PKMYT1 potency as compared to single substitution in either the 2- or 4-position of the phenol ring.

TABLE 4
Biological Data

| | | PKMYT1 |
|---|---|---|
| # | Structure | IC50 μM |
| BAA-001 | 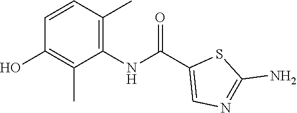 [SMILES: Cc1ccc(O)c(C)c1NC(=O)c1cnc(N)s1] | 0.327 |
| REF-001 | 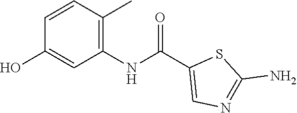 [SMILES: Cc1ccc(O)cc1NC(=O)c1cnc(N)s1] | 53.2 |
| REF-002 | 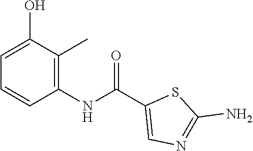 [SMILES: Cc1c(O)cccc1NC(=O)c1cnc(N)s1] | 26.8 |

### Biological Example 8: Comparison Data 2

IC50 was measured for BAA-004, REF-003, and REF-004 as described for PKMYT1 biochemical ADP-Glo enzymatic assay in Biological Example 2. See Biological Data Table 5, below.

As compared to REF-003, BAA-004 exhibits a 987-fold improvement in PKMYT1 IC50.

As compared to REF-004, BAA-004 exhibits a >1800-fold improvement in PKMYT1 IC50.

The enhancement seen when the di- ortho-substituted phenyl ring is hydroxyl substituted (i.e., a phenol) indicates that the hydroxyl substitution (meta to the amide group) confers surprising and unexpected improvements in PKMYT1 potency as compared to compounds lacking the hydroxyl substituent. Additionally, the data indicate that unsubstituted hydroxyl groups at this position lead to surprising and unexpected improvements as compared to substituted hydroxyl groups (e.g., alkyloxy).

TABLE 5
Biological Data

| | | PKMYT1 |
|---|---|---|
| # | Structure | IC50 μM |
| BAA-004 | 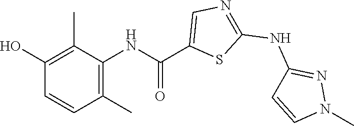 [SMILES: Cc1ccc(O)c(C)c1NC(=O)c1cnc(Nc2ccn(C)n2)s1] | 0.055 |
| REF-003 | 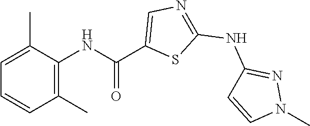 [SMILES: Cc1cccc(C)c1NC(=O)c1cnc(Nc2ccn(C)n2)s1] | 54.3 |
| REF-004 | 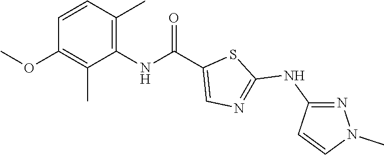 | >100 |

The foregoing has described the principles, preferred embodiments, and modes of operation of the present invention. However, the invention should not be construed as limited to the particular embodiments discussed. Instead, the above-described embodiments should be regarded as illustrative rather than restrictive. It should be appreciated that variations may be made in those embodiments by workers skilled in the art without departing from the scope of the present invention.

### REFERENCES

Publications are cited herein in order to more fully describe the state of the art to which the invention pertains. Full citations for these references are provided below.

Each of these publications is incorporated herein by reference in its entirety into the present disclosure, to the same extent as if each individual reference was specifically and individually indicated to be incorporated by reference.
Booher et al., 1997, ‘Human Myt1 Is a Cell Cycle-regulated Kinase That Inhibits Cdc2 but Not Cdk2 Activity’, J Biol Chem. 29; 272 (35): 22300-6.
Bryan et al., 2006, “304. Identifying cellular genes crucial for the reactivation of Kaposi's sarcoma-associated herpesvirus latency”, Journal of General Virology, 87 (3), 519-529.
Cai et al., 2022, “PKMYT1 regulates the proliferation and epithelial-mesenchymal transition of oral squamous cell carcinoma cells by targeting CCNA2”, Oncology Letters (2022), 23 (2), 63.
Chayka et al., 2015, “Identification and Pharmacological Inactivation of the MYCN Gene Network as a Therapeutic Strategy for Neuroblastic Tumor Cells”, Journal of Biological Chemistry, 290 (4), 2198-2212.
Chen et al., 2020, “Overexpression of PKMYT1 facilitates tumor development and is correlated with poor prognosis in clear cell renal cell carcinoma” Medical Science Monitor, 26, e926755.
Chen et al., 2021, “PKMYT1, exacerbating the progression of clear cell renal cell carcinoma, is implied as a biomarker for the diagnosis and prognosis”, Aging, 13 (24), 25778-25798.
Durocher et al., 2021, “Methods of Using Myt1 Inhibitors”, International (PCT) Patent Application Publication No. WO 2021/195782 A1.
Fassl et al., 2022, ‘CDK4 and CDK6 kinases: From basic science to cancer therapy.’ Science. 375 (6577): eabc1495.
Gallo et al., 2022, “CCNE1 amplification is synthetic lethal with PKMYT1 kinase inhibition”, Nature, 604 (7907), 749-756.
Ghelli Luserna di Rora et al., 2020, “A WEE1 family business: regulation of mitosis, cancer progression, and therapeutic target”, Journal of Hematology & Oncology, 13 (1): 126.
Gorski et al., 2020, ‘CCNE1 Amplification as a Predictive Biomarker of Chemotherapy Resistance in Epithelial Ovarian Cancer.’ Diagnostics (Basel). 5; 10 (5): 279.
He et al., 2021, “LncRNA PKMYT1AR promotes cancer stem cell maintenance in non-small cell lung cancer via activating Wnt signaling pathway”, Molecular Cancer, 20 (1), 156.
Hu et al., 2022, “. Demethylase ALKBH5 suppresses invasion of gastric cancer via PKMYT1 m6A modification”, Molecular Cancer, 21 (1), 34.
Jeong et al., 2018, “109. Protein kinase, membrane-associated tyrosine/threonine 1 is associated with the progression of colorectal cancer”, Oncology Reports, 39 (6), 2829-2836.
Jones et al., 2013, “Increased replication initiation and conflicts with transcription underlie Cyclin E-induced replication stress” Oncogene 32, 3744-3753.
Lewis et al., 2019, “Upregulation of Myt1 Promotes Acquired Resistance of Cancer Cells to Wee1 Inhibition”, Cancer research, 79 (23), 5971-5985.
Liu et al., 1997, ‘The human Myt1 kinase preferentially phosphorylates Cdc2 on threonine 14 and localizes to the endoplasmic reticulum and Golgi complex’, Mol Cell Biol. 17 (2): 571-83.
Liu et al., 2017, “PKMYT1 promoted the growth and motility of hepatocellular carcinoma cells by activating beta-catenin/TCF signaling”, Experimental Cell Research, 358 (2), 209-216.
Liu et al., 2020, “Systematic expression analysis of WEE family kinases reveals the importance of PKMYT1 in breast carcinogenesis”, Cell proliferation, 53 (2), e12741.
Long et al., 2020, “84. PKMYT1 as a potential target to improve the radiosensitivity of lung adenocarcinoma”, Frontiers in Genetics, 11, 00376.
Luo et al., 2022, “c-Myb-mediated inhibition of miR-601 in facilitating malignance of osteosarcoma via augmentation of PKMYT1”, Scientific Reports, 12 (1), 6692.
Shao et al., 2021, “The DNA damage repair-related gene PKMYT1 is a potential biomarker in various malignancies”, Translational Lung Cancer Research, 10 (12), 4600-4616.
Sun et al., 2019, “Overexpression of PKMYT1 indicates the poor prognosis and enhances proliferation and tumorigenesis in non-small cell lung cancer via activation of Notch signal pathway”, European review for medical and pharmacological sciences, 23 (10), 4210-4219.
Szychowski et al., 2022, “Discovery of an Orally Bioavailable and Selective PKMYT1 Inhibitor, RP-6306”, Journal of Medicinal Chemistry, 65 (15), 10251-10284
Toledo et al., 2015, “Genome-wide CRISPR-Cas9 Screens Reveal Loss of Redundancy between PKMYT1 and WEE1 in Glioblastoma Stem-like Cells”, Cell Reports, 13 (11), 2425-2439.
Visconti et al., 2017, “Fighting tubulin-targeting anticancer drug toxicity and resistance”, Endocrine-Related Cancer, 24 (9), T107-T117.
Wang et al., 2020, “PKMYT1 is associated with prostate cancer malignancy and may serve as a therapeutic target”, Gene, 744, 144608.
Xuan et al., 2020, “PKMYT1 aggravates the progression of ovarian cancer by targeting SIRT3”, European review for medical and pharmacological sciences, 24 (10), 5259-5266.
Yost et al., 2021, “Methods and compositions for treating cancer”, PCT Int. Appl. (2021), WO 2021202780.
Zhang et al., 2019, “Overexpressed PKMYT1 promotes tumor progression and associates with poor survival in esophageal squamous cell carcinoma”, Cancer Management and Research, 11, 7813-7824.
Zhang et al., 2020, “PKMYT1 promotes gastric cancer cell proliferation and apoptosis resistance”, OncoTargets and Therapy, 13, 7747-7757.
Zhang et al., 2022, “KDM2B mediates the Wnt/β-catenin pathway through transcriptional activation of PKMYT1 via microRNA-let-7b-5p/EZH2 to affect the development of non-small cell lung cancer”, Experimental Cell Research, 417 (2), 113208.

### Statements

Statement 1. A compound of the following formula:

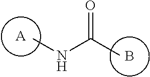

or a pharmaceutically acceptable salt or solvate thereof;
wherein:
—Ring A is:

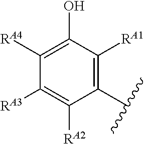

wherein:
—R^{A1} is —R^{A11};
—R^{A11} is —R^{A111}, —F, —Cl, —Br, —I, —CF₃, —CHF₂, —OH, —OR^{A111}, —OCF₃, —NH₂, —NHR^{A111}, —NR^{A111}₂, —CN, —C(═O)R^{A111}, —C(═O)OH, —C(═O)OR^{A111}, —C(═O)NH₂, —C(═O)NHR^{A111}, —C(═O)NR^{A111}₂, or —S(═O)₂R^{A111};
each —R^{A111} is independently linear or branched saturated C₁₋₄alkyl;
—R^{A2} is —R^{A22};
—R^{A22} is —R^{A222}, —F, —Cl, —Br, —I, —CF₃, —CHF₂, —OH, —OR^{A222}, —OCF₃, —NH₂, —NHR^{A222}, —NR^{A222}₂, —CN, —C(═O)R^{A222}, —C(═O)OH, —C(═O)OR^{A222}, —C(═O)NH₂, —C(═O)NHR^{A222}, —C(═O)NR^{A222}₂, or —S(═O)₂R^{A222};
each —R^{A222} is independently linear or branched saturated C₁₋₄alkyl;
—R^{A3} is —H or —R^{A33};
—R^{A33} is —R^{A333}, —F, —Cl, —Br, —I, —CF₃, —OH, —OR^{A333}, or —OCF₃;
each —R^{A333} is independently linear or branched saturated C₁₋₄alkyl;
—R^{A4} is —H or —R^{A44};
—R^{A44} is —R^{A444}, —F, —Cl, —Br, —I, —CF₃, —OH, —OR^{A444}, or —OCF₃;
each -RA444 is independently linear or branched saturated C₁₋₄alkyl;
and:
—Ring B is selected from:

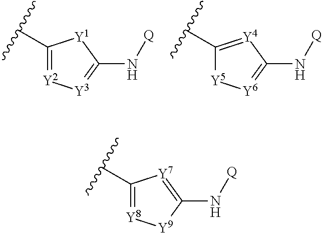

wherein:
Y¹ is S, O, NH, NR^{Y1};
Y² is CH, CR^{Y2}, or N;
Y³ is N, CH, or CR^{Y3};
Y⁴ is N, CH, or CR^{Y4};
Y⁵ is S, O, NH, NR^{Y5};
Y⁶ is N, CH, or CR^{Y6};
Y⁷ is N, CH, or CR^{Y7};
Y⁸ is N, CH, or CR^{Y8};
Y⁹ is S, O, NH, NR^{Y9};
wherein:
each —R^{Y2}, —R^{Y3}, —R^{Y4}, —R^{Y6}, —R^{Y7}, and —R^{Y8} is independently —H, —F, —Cl, —Br, —I, —R^{YY}, —CF₃, —OH, —OR^{YY}, —OCF₃, —NH₂, —NHR^{YY}, or —NR^{YY}₂;
each —R^{YY} is independently linear or branched saturated C₁₋₄alkyl;
and wherein:
each —R^{Y1}, —R^{Y5}, and —R^{Y9} is independently —R^{YYN}, —C(═O)R^{YYN}, —C(═O)OR^{YYN}, —C(═O)NH₂, —C(═O)NHR^{YYN}, —C(═O)NR^{YYN}₂, or —S(═O)₂R^{YYN};
each —R^{YYN} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl,
wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
-Q is -Q¹, -L^{Q1}-Q¹, -Q², -L^{Q2}-Q², -Q³, -L^{Q3}-Q³, -Q⁴, -L^{Q4}-Q⁴, -Q⁵, or —H;
wherein:
Q¹ is C₅₋₁₀heteroaryl;
and is:
optionally substituted on carbon with one or more groups —R^{Q1C}; and
optionally substituted on secondary nitrogen, if present, with one or more groups —R^{Q1N};
-L^{Q1}- is linear or branched saturated C₁₋₄alkylene;
Q² is non-aromatic C₃₋₇heterocyclyl;
and is:
optionally substituted on sulfur, if present, with one or two groups ═O;
optionally substituted on carbon with one or more groups -RQ20; and
optionally substituted on secondary nitrogen, if present, with one or more groups —R^{Q2N};
-L^{Q2}- is linear or branched saturated C₁₋₄alkylene;
Q³ is phenyl or naphthyl;
and is optionally substituted with one or more groups -RQ30;
L23- is linear or branched saturated C₁₋₄alkylene;
Q⁴ is C₃₋₇cycloalkyl;
and is optionally substituted with one or more groups —R^{Q4C};
-L^{Q4}- is linear or branched saturated C₁₋₄alkylene;
Q⁵ is linear or branched saturated C₁₋₆alkyl;
and is optionally substituted with one or more groups —R^{Q5C};
and wherein:
each —R^{Q1C} is independently:
—F, —Cl, —Br, —I,
—R^{Q1CC},
—R^{Q1CX}, —OR^{Q1CX},
—OH, —OR^{Q1CC},
-L^{Q1C}—OH, -L^{Q1C}-OR^{Q1CC},
—NH₂, —NHR^{Q1CC}, —NR^{Q1CC}₂, -RQ¹ CM
-L^{Q1C}—NH₂, -L^{Q1C}-NHR^{Q1CC}, -L^{Q1C}-NR^{Q1CC}₂, -L^{Q1C}-RQ¹ CM
—NHC(═O)R^{Q1CC}, —NHC(═O)OR^{Q1CC},
-L^{Q1C}—NHC(═O)R^{Q1CC}, L^{Q10}—NHC(═O)OR^{Q1CC},
—C(═O)NH₂, —C(═O)NHR^{Q1CC}, —C(═O)NR^{Q1CC}₂, —C(═O)RQ¹ CM
-L^{Q1C}-C(═O)NH₂, L^{Q1C}—C(═O)NHR^{Q1CC}, L^{Q1C}—C(═O)NR^{Q1CG}₂, -L^{Q1C}—C(═O)RQ¹ CM
—C(═O)OH, —C(═O)OR^{Q1CC},
—OC(═O)R^{Q1CC},
—OC(═O)NH₂, —OC(═O)NHR^{Q1CC}, —OC(═O)NR^{Q1CG}₂, —OC(═O)RQ¹ CM
—S(═O)₂R^{Q1CC},
—S(═O) 2NH₂, —S(═O)₂NHR^{Q1CC}, —S(═O)₂NR^{Q1CC}₂, —S(═O)₂NRQ¹ CM
—CN, or —NO2;
and two adjacent-R^{Q1C}, if present, taken together may form —(CH₂)ₙ₁—O—(CH₂)ₘ₁—or
—O—(CH₂)ₚ₁—O—, wherein: n1 is 0, 1, 2, or 3; m1 is 0, 1, 2, or 3; and p1 is 1 or 2; with the proviso that m1+n1 is 2 or 3;
wherein:
each —R^{Q1CC} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂,
—NH(CH₃), and —N(CH₃)₂;
each —R^{Q1CX} is independently linear or branched saturated C₁₋₄haloalkyl;
each -L^{Q1C}- is independently linear or branched saturated C₁₋₄alkylene;
each —R^{Q1CM} is independently non-aromatic C₃₋₁₁heterocyclyl having at least one N ring atom, and is attached via that N ring atom; and is:
optionally substituted on carbon with one or more groups —R^{Q1CMM};
optionally substituted on sulfur, if present, with one or two ═O groups; and
optionally substituted on secondary nitrogen, if present, with a group selected from: R^{Q1CMM}, —C(═O)R^{Q1CMM}, —C(═O)OR^{Q1CMM}, —C(═O)NH₂, —C(═O)NHR^{Q1CMM}; —C(═O)NR^{Q1CMM}₂, and —S(═O)₂R^{Q1CMM}; and
each —R^{Q1CMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected
from: —F, —Cl, —CH₃, —CF₃, —OH, —OCH₃, —NH₂,
—NH(CH₃), and —N(CH₃)₂;
and wherein:
each —ROIN is independently:
—RQ1NC
—R^{Q1NX},
—R^{Q1Nhet}, -LQ1N—R^{Q1Nhet},
-L^{Q1N}-OH, -L^{Q1N}-ORQ1NC
-L^{Q1N}-C(═O)R^{Q1NC},
-L^{Q1N}-C(═O)OH, -L^{Q1N}—C(═O)OR^{Q1NC},
-L^{Q1N}-C(═O)NH₂, -L^{Q1N}-C(═O)NHR^{Q1NK}, -L^{Q1N}—C(═O)NR^{Q1NG}₂, -L^{Q1N}-C(═O)RQ1NP
-L^{Q1N}-NH₂, -L^{Q1N}-NHR^{Q1NC}, -LQ1N—NR^{Q1NC}₂, -L^{Q1N}-RQ1NM
or -LQIN—NHC(═O)OR^{Q1NC};
wherein:
each —R^{Q1NG} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂; each —R^{Q1NX} is independently linear or branched saturated C₁₋₄haloalkyl;
each -L^{Q1N}- is independently linear or branched saturated C₁₋₄alkylene;
each —R^{Q1NM} is independently non-aromatic C₃₋₇heterocyclyl having at least one N ring atom, and is attached via that N ring atom; and is:
optionally substituted on carbon with one or more groups —R^{Q1CMM}; optionally substituted on sulfur, if present, with one or two ═O groups; and optionally substituted on secondary nitrogen, if present, with a group selected from: —R^{Q1NMM}, —C(═O)R^{Q1NMM}, —C(═O)OR^{Q1NMM}, —C(═O)NH₂, —C(═O)NHR^{Q1NMM}, —C(═O)NR^{Q1NMM}₂, and —S(═O)₂R^{Q1NMM}; and
each —R^{Q1NMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;

and wherein:

each —R^{Q1Nhet} is independently non-aromatic C₃₋₇heterocyclyl; and is:
optionally substituted on sulfur, if present, with one or two groups ═O;

optionally substituted on carbon with one or more groups —R^{Q1NHH} or ═O; and

optionally substituted on secondary nitrogen, if present, with a group selected from: —R^{Q1NHH}, —C(═O)R^{Q1NHH}, —C(═O)OR^{Q1NHH}, —C(═O)NH₂, —C(═O)NHR^{Q1NHH}, —C(═O)NR^{Q1NHH}₂, and —S(═O)₂R^{Q1NHH},

wherein:
each —R^{Q1NHH} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
—R^{Q1NJJ} is —R^{J1}, —R^{J2}, -LJ-R^{J2}, —R^{J3}, -L^{J}-R^{J3}, —R^{J4}, -L^{J}-R^{J4}, —R^{J5}, or -L^{J}-R^{J5};
—R^{J1} is linear or branched saturated C₁₋₆alkyl; and is optionally substituted with one or more groups selected from: —F, —OH, —OR^{J}, —O-phenyl, —C(═O)OH, —C(═O)OR^{JJ}, —NH₂, —NHR^{J}, and —NR^{JJ}₂;
each —R^{J2} is independently C₃₋₆cycloalkyl; and is optionally substituted with one or more groups selected from: —F, —R^{J}, —CF₃, —OH, —OR^{J}, —NH₂, —NHR^{J}, and —NR^{J2};
each —R^{J3} is independently non-aromatic C₃₋₇heterocyclyl; and is:
optionally substituted on sulfur, if present, with one or two groups ═O;
optionally substituted on carbon with one or more groups selected from —F, —R^{J}, —CF₃, —OH, —OR^{JJ}, —NH₂, —NHR^{JJ}, and —NR^{JJ}₂; and
optionally substituted on secondary nitrogen, if present, with a group selected from: —R^{J}, —C(═O)R″, —C(═O)OR^{JJ}, and —S(═O)₂RUJ;
each —R^{J4} is phenyl; and is optionally substituted with one or more groups selected from: —F, —Cl, —R^{J}, —CF₃, —OH, —OR^{J}, —NH₂, —NHR^{J}, and —NR^{J2};
each —R^{J5} is independently C5 cheteroaryl; and is:
optionally substituted on carbon with one or more groups selected from —F, —R^{J}, —CF₃, —OH, —OR^{J}, —NH₂, —NHR^{J}, and -NRJ2; and optionally substituted on secondary nitrogen, if present, with a group selected from: —R^{J}, —C(═O)R^{J}, —C(═O)OR^{J}, and —S(═O)₂R^{JJ};
each -L′- is independently linear or branched saturated C₁₋₄alkylene, and is optionally substituted with one or more-F;
each —R^{J} is linear or branched saturated C₁₋₄alkyl;
and wherein:
—R^{Q1NK} is —R^{K1}, —R^{K2}, -L^{K}-R^{K2}, —R^{K3}, -L^{K}-R^{K3}, —R^{K4}, -L^{K}-R^{K4}, —R^{K5}, or -L^{K}-RK5;
—R^{K1} is linear or branched saturated C1.7alkyl; and is optionally substituted with one or more groups selected from: —F, —OH, —OR^{KK}, —OCH₂CH₂OCH₃, —O-phenyl, —C(═O)OH, —C(═O)OR^{KK}, —NH₂, —NHR^{KK}, and —NR^{KK2};
each —R^{K2} is independently C₃₋₆cycloalkyl; and is optionally substituted with one or more groups selected from: —F, —R^{KK}, —CF₃, —OH, —OR^{KK}, —NH₂, —NHR^{KK}, and —NR^{KK2};
each —R^{K3} is independently non-aromatic C₃₋₇heterocyclyl; and is:
optionally substituted on carbon with one or more groups selected from —F, —R^{KK}, —CF₃, —OH, —OR^{KK}, —NH₂, —NHR^{KK}, and —NR^{KK2}; and optionally substituted on secondary nitrogen, if present, with a group selected from: —R^{KK}, —C(═O)R^{KK}, —C(═O)OR^{KK}, and —S(═O)₂R^{KK};
each —R^{K4} is phenyl; and is optionally substituted with one or more groups selected from: —F, —Cl, —R^{KK}, —CF₃, —OH, —OR^{KK}, —NH₂, —NHR^{KK}, and —NR^{KK2};
each —R^{K5} is independently C₅₋₆heteroaryl; and is:
optionally substituted on carbon with one or more groups selected from —F, —R^{KK}, —CF₃, —OH, —OR^{KK}, —NH₂, —NHR^{KK}, and —NR^{KK2}; and optionally substituted on secondary nitrogen, if present, with a group selected from: —R^{KK}, —C(═O)R^{KK}, —C(═O)OR^{KK}, and —S(═O)₂R^{KK};
each -L^{K}- is linear or branched saturated C₁₋₄alkylene, and is optionally substituted with one or more-F;
each —R^{KK} is linear or branched saturated C₁₋₄alkyl;
and wherein:
—R^{Q1NP} is non-aromatic C₃₋₁₁heterocyclyl having at least one N ring atom, and is attached via that N ring atom; and is:

optionally substituted on sulfur, if present, with one or two ═O groups;
optionally substituted on carbon with one or more groups selected from —R^{Q1NPP}, —F, —OH, —OR^{Q1NPP}, and ═O; and
optionally substituted on secondary nitrogen, if present, with a group selected from: —R^{Q1NPP}, —R^{Q1NPPX}, —C(═O)R^{Q1NPP}, —C(═O)OR^{Q1NPP}, —C(═O)NH₂, —C(═O)NHR^{Q1NPP}, —C(═O)NR^{Q1NPP}₂, and —S(═O)₂R^{Q1NPP};
each —R^{Q1NPP} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
—R^{Q1NPPX} is linear or branched saturated C₁₋₄haloalkyl;
and wherein:
each —R92C is independently:
—F,
—R^{Q2CC}, —R^{Q2CX},
—OH, —OR^{Q2CC}, —OR^{Q2CX},
—NH₂, —NHR^{Q2CC}, —NR^{Q2CC}₂, -RQ² CM,
—NHC(═O)R^{Q2CC}, —NHC(═O)OR^{Q2CC},
—C(═O)NH₂, —C(═O)NHR^{Q2CC}, —C(═O)NR^{Q2CC}₂, —C(═O)R^{Q2CM},
—C(═O)OH, —C(═O)OR^{Q2CC},
—OC(═O)R^{Q2CC},
—OC(═O)NH₂, —OC(═O)NHR^{Q2CC}, —OC(═O)NR^{Q2CG}₂, —OC(═O)R^{Q2CM}, or
═O;
wherein:
each —R^{Q2CC} is independently linear or branched saturated C₁₋₄alkyl, C₃₋₆cycloalkyl,
C₃₋₆cycloalkyl-C₁₋₃alkyl, phenyl, C₃₋₇heterocyclyl, phenyl-C₁₋₃alkyl, C₅₋₆heteroaryl, or
C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂,
—NH(CH₃), and —N(CH₃)₂;
each —R^{Q2CX}, is independently linear or branched saturated C₁₋₄haloalkyl;
each -RQ² CM is independently non-aromatic C₃₋₁₁heterocyclyl having at least one N ring atom, and is attached via that N ring atom; and is:
optionally substituted on carbon with one or more groups —R^{Q2CMM};
optionally substituted on sulfur, if present, with one or two ═O groups; and
optionally substituted on secondary nitrogen, if present, with a group selected from: —R^{Q2CMM}, —C(═O)R^{Q2CMM}, —C(═O)OR^{Q2CMM}, —C(═O)NH₂, —C(═O)NHR^{Q2CMM}, —C(═O)NR^{Q2CMM}₂, and —S(═O)₂R^{Q2CMM}; and
each -R92CMM is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
each —R^{Q2N} is independently:
—RO2NC,
—C(═O)R^{Q2NC},
—C(═O-L^{Q2N}—OH, —C(═O)-L32N-ORQ2NC
—C(═O)-L^{Q2N}-NH₂, —C(═O)-L^{Q2N}-NHR^{Q2NC}, —C(═O)-L^{Q2N}-NR^{Q2NC}₂, —C(═O)-L^{Q2N}-R^{Q2NM},
—C(═O)ORQ2NC—-L^{Q2N}-NH₂, -LO2N-NHRQ2NC, -L^{Q2N}-NR^{Q2NC}₂, -LQ2N—R^{Q2NM},
—C(═O)NH₂, —C(═O)NHR^{Q2NC}, —C(═O)NR^{Q2NC}₂, —C(═O)R^{Q2NM},
-L^{Q2N}-C(═O)NH₂, -LO2N—C(═O)NHR^{Q2NC}, -L^{Q2N}-C(═O)NR^{Q2NC}₂, -L^{Q2N}-C(═O)ROZNM, or
—S(═O)₂RQ2NC;
wherein:
each -RQ2NC is independently linear or branched saturated C₁₋₆alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl,
wherein C₁₋₆alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
each -LO2N- is independently linear or branched saturated C₁₋₄alkylene;
each -RO2NM is independently non-aromatic C₃₋₁₁heterocyclyl having at least one N ring
atom, and is attached via that N ring atom; and is:
optionally substituted on carbon with one or more groups -RQ2NMM.
optionally substituted on sulfur, if present, with one or two ═O groups; and
optionally substituted on secondary nitrogen, if present, with a group selected from: —R^{Q2NM}, —C(═O)R^{Q2NM}, —C(═O)OR^{Q2NM}, —C(═O)NH₂, —C(═O)NHRQ2NM —C(═O)NRQ2NMM2, and —S(═O)₂R^{Q2NMM}; and
each —R^{Q2NMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
each —R93C is independently:
—F, —Cl, —Br, —I,
—R^{Q3CC},
—R^{Q3CX} CX—OR^{Q3CX} CX
—OH, —OR^{Q3CC},
-L^{Q3C}—OH, -LO3C—OR^{Q3CX} CC
—NH₂, —NHR^{Q3CC}, —NRO3CC2, -RQ³ CM
-L^{Q3C}-₂, -L^{Q3CC}-NHR^{Q3CXCC}—NR^{Q3CX} CC₂, -L^{Q3C}-Q³ CM,
—NHC(═O)R^{Q3CC}, —NHC(═O)OR^{Q3CC},
-L^{Q3C}-C(═O)RO3CC, -L^{Q3C}—NHC(═O)OR^{Q3CC},
—C(═O)NH₂, —C(═O)NHR^{Q3CC}, —C(═O)NR^{Q3CXCC}₂, —C(═O)RQ³ CM
-L^{Q3C}-C(═O)NH₂, -LQ3C—C(═O)NHR^{Q3CC}, -L^{Q3C}-C(═O)NR^{Q3CXCC}₂, -L^{Q3C}-C(═O)RQ³ CM
—C(═O)OH, —C(═O)OR^{G3CC},
—OC(═O)R^{Q3CC},
—OC(═O)NH₂, —OC(═O)NHR03CC, —OC(═O)NR03CC2, —OC(═O)R03 CM,
—CN, or —NO₂;
and two adjacent-RO3C, if present, taken together may form —(CH₂)ₙ₃—O—(CH₂)ₘ₃—or
—O—(CH₂)ₚ₃—O—, wherein: n3 is 0, 1, 2, or 3; m3 is 0, 1, 2, or 3; and p3 is 1 or 2; with the proviso that m3+n3 is 2 or 3;
wherein:
each —R^{Q3CC} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂,
—NH(CH₃), and —N(CH₃)₂;
each —R^{Q3CX} is independently linear or branched saturated C₁₋₄haloalkyl;
each -L^{Q3C}- is independently linear or branched saturated C₁₋₄alkylene;
each -RQ³ CM is independently non-aromatic C₃₋₁₁heterocyclyl having at least one N ring atom, and is attached via that N ring atom; and is:
optionally substituted on carbon with one or more groups —R^{Q3CCMM}, optionally substituted on sulfur, if present, with one or two ═O groups; and
optionally substituted on secondary nitrogen, if present, with a group selected from: —R^{Q3CCMM}, —C(═O)R^{Q3CCMM}, —C(═O)OR^{Q3CCMM}, —C(═O)NH₂, —C(═O))NHR^{Q3CCMM}, —C(═ONR^{Q3CMM}₂, and —S(═O)₂R^{Q3CCMM}; and
each -R93CMM is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
each —R^{Q4C} is independently:
—F,
—RQ4CC-RQ4CX
—OH, —OR^{Q4CC}, -ORQ4CX
—NH₂, —NHR^{Q4CC}, —NR^{Q4CC}₂, -RQ⁴ CM
—NHC(═O)R^{Q4CC}, —NHC(═O)OR^{Q4CC},
—C(═O)NH₂, —C(═O)NHR^{Q4CC}, —C(═O)NR^{Q4CC}₂, —C(═O)RQ⁴ CM
—C(═O)OH, —C(═O)OR^{Q4CC},
—OC(═O)RQ4CC
—OC(═O)NH₂, —OC(═O)NHR^{Q4CC}, —OC(═O)NRQ4CG2, —OC(═O)RQ⁴ CM, or
═O;
wherein:
each —R^{Q4CC} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
each —R04CX is independently linear or branched saturated C₁₋₄haloalkyl;
each -RQ⁴ CM is independently non-aromatic C₃₋₁₁heterocyclyl having at least one N ring atom, and is attached via that N ring atom; and is:
optionally substituted on carbon with one or more groups -RQ4CMM.
optionally substituted on sulfur, if present, with one or two ═O groups; and
optionally substituted on secondary nitrogen, if present, with a group selected from: —R^{Q4CMM}, —C(═O)R^{Q4CMM}, —C(═O)OR^{Q4CMM}, —C(═O)NH₂, —C(═O)NHRQ4CMM —C(═ONR^{Q4CMM}₂, and —S(═O)₂R^{Q4CMM};
each —R^{Q4CMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
each —R05° C. is independently:
—F, —OH, —OR^{Q5CC}, —OCF₃,
—NH₂, —NHR^{Q5CC}, —NR^{Q5CC}₂, -RQ⁵ CM
—NHC(═O)R^{Q5CC}, —NHC(═O)OR^{Q5CC},
—C(═O)NH₂, —C(═O)NHR^{Q5CC}, —C(═O)NR^{Q5CC}₂, —C(═O)RQ⁵ CM
—C(═O)OH, —C(═O)OR^{Q5CC},
—OC(═O)R^{Q5CC},
—OC(═O)NH₂, —OC(═O)NHR^{Q5CC}, —OC(═O)NR^{Q5CC}₂, or —OC(═O)R^{Q5CM},
wherein:
each —R^{Q5CC} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
each —RO5 CM is independently non-aromatic C₃₋₁₁heterocyclyl having at least one N ring atom, and is attached via that N ring atom; and is:
optionally substituted on carbon with one or more groups —R^{Q5CMM},
optionally substituted on sulfur, if present, with one or two ═O groups; and
optionally substituted on secondary nitrogen, if present, with a group selected from: —R^{Q5CMM}, —C(═O)R^{Q5CMM}, —C(═O)OR^{Q5CMM}, —C(═O)NH₂, —C(═O)NHR^{Q5CMM}, —C(═O)NR^{Q5CMM}₂, and —S(═O)₂R^{Q5CMM};
each —R^{Q5CMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂.

Statement 2. A pharmaceutical composition comprising:
a compound according to statement 1, or
a pharmaceutically acceptable salt or solvate thereof, and
a pharmaceutically acceptable carrier or diluent.

Statement 3. A method of preparing a pharmaceutical composition comprising the step of:
mixing a compound according to statement 1, or
a pharmaceutically acceptable salt or solvate thereof, and
a pharmaceutically acceptable carrier or diluent.

Statement 4. A method of inhibiting PKMYT1 kinase, in vitro or in vivo, comprising contacting the kinase with an effective amount of a compound according to statement 1, or a pharmaceutically acceptable salt or solvate thereof.

Statement 5. A method of inhibiting PKMYT1 kinase function in a cell, in vitro or in vivo, comprising contacting the cell with an effective amount of a compound according to statement 1, or a pharmaceutically acceptable salt or solvate thereof.

Statement 6. A compound according to statement 1, or a pharmaceutically acceptable salt or solvate thereof, for use in a method of treatment of the human or animal body by therapy.

Statement 7. A compound according to statement 1, or a pharmaceutically acceptable salt or solvate thereof, for use in a method of treatment of a disorder of the human or animal body that is ameliorated by the inhibition of PKMYT1 kinase.

Statement 8. Use of a compound according to statement 1, or a pharmaceutically acceptable salt or solvate thereof, in the manufacture of a medicament for the treatment of a disorder of the human or animal body that is ameliorated by the inhibition of PKMYT1 kinase.

Statement 9. A method of treatment of a disorder of the human or animal body that is ameliorated by the inhibition of PKMYT1 kinase, comprising administering to a subject in need of treatment a therapeutically-effective amount of a compound according to statement 1, or a pharmaceutically acceptable salt or solvate thereof.

Statement 10. A compound according to statement 1, or a pharmaceutically acceptable salt or solvate thereof, for use in a method of treatment of a proliferative disorder.

Statement 11. Use of a compound according to statement 1, or a pharmaceutically acceptable salt or solvate thereof, in the manufacture of a medicament for the treatment of a proliferative disorder.

Statement 12. A method of treatment of a proliferative disorder of the human or animal body, comprising administering to a subject in need of treatment a therapeutically-effective amount of a compound according to statement 1, or a pharmaceutically acceptable salt or solvate thereof.

Statement 13. A compound, salt, or solvate for use according to statement 10,
use according to statement 11, or
a method according to statement 12,
wherein the proliferative disorder is characterised by, or further characterised by:
inappropriate activity and/or expression of PKMYT1, CCNE1, FBXW7, or PPP2A or one of its subunits.

Statement 14. A compound, salt, or solvate for use according to statement 10,
use according to statement 11, or
a method according to statement 12,
wherein the proliferative disorder is cancer.

Statement 15. A compound, salt, or solvate for use according to statement 10,
use according to statement 11, or
a method according to statement 12,
wherein the proliferative disorder is: endometrial cancer, uterine cancer, ovarian cancer, breast cancer, gastric cancer, bladder cancer, pancreatic cancer, mesothelioma, kidney cancer, stomach cancer, esophageal cancer, colorectal cancer, glioblastoma, or lung cancer.

1. A compound of the following formula: 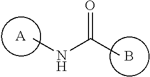
or a pharmaceutically acceptable salt or solvate thereof;
wherein:
—Ring A is: 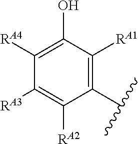
wherein:
—R^{A1} is —R^{A11};
—R^{A11} is —R^{A111}, —F, —Cl, —Br, —I, —CF₃, —CHF₂, —OH, —OR^{A111}, —OCF₃, —NH₂, —NHR^{A111}, —NR^{A111}₂, —CN, —C(═O)R^{A111}, —C(═O)OH, —C(═O)OR^{A111}, —C(═O)NH₂, —C(═O)NHR^{A111}, —C(═O)NR^{A111}₂, or —S(═O)₂R^{A111};
each —R^{A111} is independently linear or branched saturated C₁₋₄alkyl;
—R^{A2} is —R^{A22};
—R^{A22} is —R^{A222}, —F, —Cl, —Br, —I, —CF₃, —CHF₂, —OH, —OR^{A222}, —OCF₃, —NH₂,
—NHR^{A222}, —NR^{A222}₂, —CN, —C(═O)R^{A222}, —C(═O)OH, —C(═O)OR^{A222}, —C(═O)NH₂,
—C(═O)NHR^{A222}, —C(═O)NR^{A222}₂, or —S(═O)₂R^{A222};
each —R^{A222} is independently linear or branched saturated C₁₋₄alkyl;
—R^{A3} is —H or —R^{A33};
—R^{A33} is —R^{A333}, —F, —Cl, —Br, —I, —CF₃, —OH, —OR^{A333}, or —OCF₃;
each —R^{A333} is independently linear or branched saturated C₁₋₄alkyl;
—R^{A4} is —H or —R^{A44};
—R^{A44} is —R^{A444}, —F, —Cl, —Br, —I, —CF₃, —OH, —OR^{A444}, or —OCF₃;
each —R^{A444} is independently linear or branched saturated C₁₋₄alkyl;
and:
—Ring B is selected from: 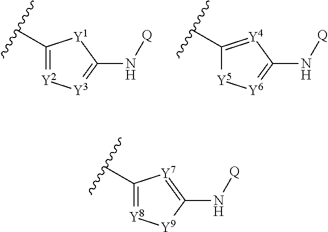
wherein:
Y¹ is S, O, NH, or NR^{Y1};
Y² is CH, CR^{Y2}, or N;
Y³ is N, CH, or CR^{Y3};
Y⁴ is N, CH, or CR^{Y4};
Y⁵ is S, O, NH, or NR^{Y5};
Y⁶ is N, CH, or CR^{Y6};
Y⁷ is N, CH, or CR^{Y7};
Y⁸ is N, CH, or CR^{Y8};
Y⁹ is S, O, NH, or NR^{Y9};
wherein:
each —R^{Y2}, —R^{Y3}, —R^{Y4}, —R^{Y6}, —R^{Y7}, and —R^{Y8} is independently —H, —F, —Cl, —Br, —I, —R^{YY}, —CF₃, —OH, —OR^{YY}, —OCF₃, —NH₂, —NHR^{YY}, or —NR^{YY}₂;
each —R^{YY} is independently linear or branched saturated C₁₋₄alkyl;
and wherein:
each —R^{Y1}, —R^{Y5}, and —R^{Y9} is independently —R^{YYN}, —C(═O)R^{YYN}, —C(═O)OR^{YYN}, —C(═O)NH₂, —C(═O)NHR^{YYN}, —C(═O)NR^{YYN}₂, or —S(═O)₂R^{YYN};
each —R^{YYN} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl,
wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂,
—NH(CH₃), and —N(CH₃)₂;
and wherein:
-Q is -Q¹, -L^{Q1}-Q¹, -Q², -L^{Q2}-Q², -Q³, -L^{Q3}-Q³, -Q⁴, -L^{Q4}-Q⁴, -Q⁵, or —H;
wherein:
Q¹ is C₅₋₁₀heteroaryl;
and is:
optionally substituted on carbon with one or more groups —R^{Q1C}; and
optionally substituted on secondary nitrogen, if present, with one or more groups —R^{Q1N};
-L^{Q1}- is linear or branched saturated C₁₋₄alkylene;
Q² is C₃₋₁₀heterocyclyl;
and is:
optionally substituted on sulfur, if present, with one or two groups ═O;
optionally substituted on carbon with one or more groups —R^{Q2C}; and
optionally substituted on secondary nitrogen, if present, with one or more groups —R^{Q2N};
-L^{Q2}- is linear or branched saturated C₁₋₄alkylene;
Q³ is phenyl or naphthyl;
and is optionally substituted with one or more groups —R^{Q3C};
-L^{Q3}- is linear or branched saturated C₁₋₄alkylene;
Q⁴ is C₃₋₁₀carbocyclyl;
and is optionally substituted with one or more groups —R^{Q4C};
-L^{Q4}- is linear or branched saturated C₁₋₄alkylene;
Q⁵ is linear or branched saturated C₁₋₆alkyl;
and is optionally substituted with one or more groups —R^{Q5C};
and wherein:
each —R^{Q1C} is independently:
—F, —Cl, —Br, —I,
—R^{Q1CX}, —OR^{Q1CX},
—OH, —OR^{Q1CC},
-L^{Q1C}—OH, -L^{Q1C}-OR^{Q1CC},
—NH₂, —NHR^{Q1CC}, —NR^{Q1CC}₂, —R^{Q1CM},
-L^{Q1C}—NH₂, -L^{Q1C}-NHR^{Q1CC}, -L^{Q1C}—NR^{Q1CC}₂, -L^{Q1C}-R^{Q1CM},
—NHC(═O)R^{Q1CC}, —N(R^{Q1CC})C(═O)R^{Q1CC}, —NHC(═O)OR^{Q1CC},
-L^{Q1C}—NHC(═O)R^{Q1CC}, -L^{Q1C}—NHC(═O)OR^{Q1CC},
—C(═O)NH₂, —C(═O)NHR^{Q1CC}, —C(═O)NR^{Q1CC}₂, —C(═O)R^{Q1CM}, ═O
—NHC(═O)NH₂, —NHC(═O)NHR^{Q1CC}, —NHC(═O)NR^{Q1CC}₂, —NHC(═O)R^{Q1CM},
-L^{Q1C}—C(═O)NH₂, L^{Q1C}—C(═O)NHR^{Q1CC}, -L^{Q1C}—C(═O)NR^{Q1CC}₂, L^{Q1C}—C(═O)RQ¹ CM
—C(═O)OH, —C(═O)OR^{Q1CC},
—OC(═O)R^{Q1CC},
—OC(═O)NH₂, —OC(═O)NHR^{Q1CC}, —OC(═O)NR^{Q1CG}₂, —OC(═O)RQ¹ CM
—S(═O)₂R^{Q1CC}, —S(═O)₂R^{Q1CX},
—S(═O) 2NH₂, —S(═O)₂NHR^{Q1CC}, —S(═O)₂NR^{Q1CC}₂, —S(═O)₂RQ¹ CM
—NHS(═O)R^{Q1CC}, —NHS(═O)₂R^{Q1CC},
—CN, —C≡CH, or —NO₂;
and two adjacent-R^{Q1C}, if present, taken together may form —(CH₂)ₙ₁—O—(CH₂)ₘ₁—or
—O—(CH₂)ₚ₁—O—, wherein: n1 is 0, 1, 2, or 3; m1 is 0, 1, 2, or 3; and p1 is 1 or 2; with the proviso that m1+n1 is 2 or 3;
wherein:
each —R^{Q1CC} is independently linear or branched saturated C₁₋₆alkyl, C₂₋₆alkenyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₆alkyl is optionally substituted with —OH, —CN or —OCH₃, and each cycloalkyl, phenyl and heteroaryl is optionally substituted with one or more groups selected
from: —F, —Cl, —C₁₋₄alkyl, —CHF₂, —CF₃, —OH, —OCH₃, —NH₂,
—NH(CH₃), and —N(CH₃)₂;
each —R^{Q1CX} is independently linear or branched saturated C₁₋₄haloalkyl;
each -L^{Q1C}- is independently linear or branched saturated C₁₋₄alkylene;
each —R^{Q1CM} is independently non-aromatic C₃₋₁₁heterocyclyl having at least one N ring atom, and is attached via that N ring atom; and is:
optionally substituted on carbon with one or more groups —R^{Q1CMM}; optionally substituted on sulfur, if present, with one or two ═O groups; and
optionally substituted on secondary nitrogen, if present, with a group selected from: R^{Q1CMM}, —C(═O)R^{Q1CMM}, —C(═O)OR^{Q1CMM}, —C(═O)NH₂, —C(═O)NHR^{Q1CMM}; —C(═O)NR^{Q1CMM}₂, and —S(═O)₂R^{Q1CMM}; and
each —R^{Q1CMM} is independently —F, —NH₂, linear or branched saturated C₁₋₄alkyl, C₁₋₄alkyIOC(═O)NH—, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂,
—NH(CH₃), and —N(CH₃)₂;
and wherein:
each —R^{Q1N} is independently:
—R^{Q1NC}-L^{Q1N}-R^{Q1NC}; —R^{Q1NC},
—R^{Q1Nhet}, -L^{Q1N}-R^{Q1Nhet},
-L^{Q1N}-OH, -L^{Q1N}-OR^{Q1NC},
-L^{Q1N}—C(═O)R^{Q1NC},
—S(═O)₂R^{Q1NC},
-L^{Q1N}-C(═O)OH, -L^{Q1N}—C(═O)OR^{Q1NC},
-L^{Q1N}-C(═O)NH₂, -L^{Q1N}-C(═O)NHR^{Q1NK}, -L^{Q1N}—C(═O)NR^{Q1NC}₂, -L^{Q1N}-C(═O)R^{Q1NP},
-L^{Q1N}-NH₂, -L^{Q1N}-NHR^{Q1NC}, -L^{Q1N}-NR^{Q1NC}₂, -L^{Q1N}-R^{Q1NM};
or -L^{Q1N}—NHC(═O)OR^{Q1NC};
wherein:
each —R^{Q1NC} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl,
wherein each C₁₋₄alkyl is optionally substituted by —F, —OH, -CEN, —SO₂—CH₃, or —OCH₃,
wherein each cycloalkyl, phenyl and heteroaryl is optionally substituted with one or more groups selected from: —F, —Cl, —Br, linear or branched saturated C₁₋₄alkyl, —CHF₂, —CF₃, —OH, —OCH₃, —CH₂—O—CH₃, —OCH₂CH₃, —C(═O)—NH—phenyl, —NH₂, —N H(CH₃), and —N(CH₃)₂; wherein C₁₋₄alkyl and phenyl are independently optionally substituted by —CH₃ or —OH;
each —R^{Q1NX} is independently linear or branched saturated C₁₋₄haloalkyl;
each -L^{Q1N}- is independently linear or branched saturated C₁₋₄alkylene; wherein C₁₋₄alkylene is optionally substituted by —OH or —OMe
each —R^{Q1NM} is independently non-aromatic C₃₋₇heterocyclyl having at least one N ring atom, and is attached via that N ring atom; and is:
optionally substituted on carbon with one or more groups —R^{Q1NMM},
optionally substituted on sulfur, if present, with one or two ═O groups; and
optionally substituted on secondary nitrogen, if present, with a group selected from: —R^{Q1NMM}, —C(═O)R^{Q1NMM}, —C(═O)OR^{Q1NMM}, —C(═O)NH₂, —C(═O)NHR^{Q1NMM}, —C(═O)NR^{Q1NMM}₂, and —S(═O)₂R^{Q1NMM}; and
each —R^{Q1NMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl,
wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
each —R^{Q1Nhet} is independently non-aromatic C₃₋₇heterocyclyl; and is:
optionally substituted on sulfur, if present, with one or two groups ═O;
optionally substituted on carbon with one or more groups —R^{Q1NHH} or ═O; and
optionally substituted on secondary nitrogen, if present, with a group selected from: —R^{Q1NHH}, —C(═O)R^{Q1NJJ}, —C(═O)OR^{Q1NHH}, —C(═O)NH₂, —C(═O)NHR^{Q1NHH}, —C(═O)NR^{Q1NHH}₂, and —S(═O)₂R^{Q1NHH},
wherein:
each —R^{Q1NHH} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
—R^{Q1NJJ} is —R^{J1}, —R^{J2}, -L^{J}-R^{J2}, —R^{J3}, -L^{J}-R^{J3}, —R^{J4}, -L^{J}-R^{J4}, —R^{J5}, or -L^{J}-R^{J5};
—R^{J1} is linear or branched saturated C₁₋₆alkyl; and is optionally substituted with one or more groups selected from: —F, —OH, —OR^{JJ}, —O-phenyl, —C(═O)OH, —C(═O)OR^{JJ}, —NH₂, —NHR^{J}, and —NR^{JJ}₂;
each —R^{J2} is independently C₃₋₆cycloalkyl; and is optionally substituted with one or more groups selected from: —F, —R^{J}, —CF₃, —OH, —OR^{J}, —NH₂, —NHR^{J}, and —NR^{J2};
each —R^{J3} is independently non-aromatic C₃₋₇heterocyclyl; and is:
optionally substituted on sulfur, if present, with one or two groups ═O;
optionally substituted on carbon with one or more groups selected from —F, —R^{J}, —CF₃, —OH, —OR^{J}, —NH₂, —NHR^{J}, and —NR^{JJ}₂; and
optionally substituted on secondary nitrogen, if present, with a group selected from: —R^{JJ}, —C(═O)R^{J}, —C(═O)OR^{JJ}, and —S(═O)₂R^{JJ};
each —R^{J4} is phenyl; and is optionally substituted with one or more groups selected from: —F, —Cl, —R^{J}, —CF₃, —OH, —OR^{JJ}, —NH₂, —NHR^{J}, and —NR^{JJ}₂;
each —R^{J5} is independently C₅₋₆heteroaryl; and is:
optionally substituted on carbon with one or more groups selected from —F, —R^{J}, —CF₃, —OH, —OR^{J}, —NH₂, —NHR^{JJ}, and —NR^{JJ}₂; and
optionally substituted on secondary nitrogen, if present, with a group selected from: —R^{J}, —C(═O)R^{JJ}, —C(═O)OR^{J}, and —S(═O)₂R^{JJ};
each -L^{J}- is independently linear or branched saturated C₁₋₄alkylene, and is optionally substituted with one or more —F;
each —R^{J} is linear or branched saturated C₁₋₄alkyl;
and wherein:
—R^{Q1NK} is —R^{K1}, —R^{K2}, -LKR^{K2}, —R^{K3}, —K—R^{K3}, —R^{K4}, -L^{K}-R^{K4}, —R^{K5}, or -L^{K}-R^{K5};
—R^{K1} is linear or branched saturated C₁₋₇alkyl; and is optionally substituted with one or more groups selected from: —F, —OH, —OR^{KK}, —OCH₂CH₂OCH₃, —O-phenyl, —C(═O)OH, —C(═O)OR^{KK}, —NH₂, —NHR^{KK}, and —NR^{KK}₂;
each —R^{K2} is independently C₃₋₆cycloalkyl; and is optionally substituted with one or more groups selected from: —F, —R^{KK}, —CF₃, —OH, —OR^{KK}, —NH₂, —NHR^{KK}, and —NR^{KK}₂;
each —R^{K3} is independently non-aromatic C₃₋₇heterocyclyl; and is:
optionally substituted on carbon with one or more groups selected from —F, —R^{KK}, —CF₃, —OH, —OR^{KK}, —NH₂, —NHR^{KK}, and —NR^{KK}₂; and
optionally substituted on secondary nitrogen, if present, with a group selected from: —R^{KK}, —C(═O)R^{KK}, —C(═O)OR^{KK}, and —S(═O)₂R^{KK};
each —R^{K4} is phenyl; and is optionally substituted with one or more groups selected from: —F, —Cl, —R^{KK}, —CF₃, —OH, —OR^{KK}, —NH₂, —NHR^{KK}, and —NR^{KK}₂;
each —R^{K5} is independently C₅₋₆heteroaryl; and is:
optionally substituted on carbon with one or more groups selected from —F, —R^{KK}, —CF₃, —OH, —OR^{KK}, —NH₂, —NHR^{KK}, and —NR^{KK}₂; and
optionally substituted on secondary nitrogen, if present, with a group selected from: —R^{KK}, —C(═O)R^{KK}, —C(═O)OR^{KK}, and —S(═O)₂R^{KK};
each -L^{K}- is linear or branched saturated C₁₋₄alkylene, and is optionally substituted with one or more —F;
each —R^{KK} is linear or branched saturated C₁₋₄alkyl;
and wherein:
—R^{Q1NP} is non-aromatic C₃₋₁₁heterocyclyl having at least one N ring atom, and is attached via that N ring atom; and is:
optionally substituted on sulfur, if present, with one or two ═O groups;
optionally substituted on carbon with one or more groups selected from —R^{Q1NPP}, —F, —OH, —OR^{Q1NPP}, and ═O; and
optionally substituted on secondary nitrogen, if present, with a group selected from: —R^{Q1NPP}, —R^{Q1NPPX}, —C(═O)R^{Q1NPP}, —C(═O)OR^{Q1NPP}, —C(═O)NH₂, —C(═O)NHR^{Q1NPP}, —C(═O)NR^{Q1NPP}₂, and —S(═O)₂R^{Q1NPP};
each —R^{Q1NPP} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —F, —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
—R^{Q1NPPX} is linear or branched saturated C₁₋₄haloalkyl;
and wherein:
each —R^{Q2C} is independently:
—F,
—R^{Q2CC}, —R^{Q2CX},
—OH, —OR^{Q2CC}, —OR^{Q2CX},
—NH₂, —NHR^{Q2CC}, —NR^{Q2CC}₂, —R^{Q2CM},
—NHC(═O)R^{Q2CC}, —NHC(═O)OR^{Q2CC},
—C(═O)NH₂, —C(═O)NHR^{Q2CC}, —C(═O)NR^{Q2CC}₂, —C(═O)R^{Q2CM},
—C(═O)OH, —C(═O)OR^{Q2CC},
—OC(═O)R^{Q2CC},
—OC(═O)NH₂, —OC(═O)NHR^{Q2CC}, —OC(═O)NR^{Q2CG}₂, —OC(═O)R^{Q2CM}, or ═O;
wherein:
each —R^{Q2CC} is independently linear or branched saturated C₁₋₄alkyl, C₃₋₆cycloalkyl, C₃₋₆cycloalkyl-C₁₋₃alkyl, phenyl, C₃₋₇heterocyclyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
each —R^{Q2CX} is independently linear or branched saturated C₁₋₄haloalkyl;
each —RO2 CM is independently non-aromatic C₃₋₁₁heterocyclyl having at least one N ring atom, and is attached via that N ring atom; and is:
optionally substituted on carbon with one or more groups —R^{Q2CMM};
optionally substituted on sulfur, if present, with one or two ═O groups; and
optionally substituted on secondary nitrogen, if present, with a group selected from: —R^{Q2CMM}, —C(═O)R^{Q2CMM}, —C(═O)OR^{Q2CMM}, —C(═O)NH₂, —C(═O)NHR^{Q2CMM}, —C(═O)NR^{Q2CMM}₂, and —S(═O)₂R^{Q2CMM}; and
each —R^{Q2CMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl,
wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
each —R92N is independently:
—R^{Q2NC},
═O, —C(═O)R^{Q2NC},
—C(═O)-L^{Q2N}—OH, —C(═O)-L^{Q2N}-OR^{Q2NC},
—C(═O)-L^{Q2N}-NH₂, —C(═O)-L^{Q2N}-NHR^{Q2NC}, —C(═O)-L^{Q2N}-NR^{Q2NC}₂, —C(═O)-L^{Q2N}-R^{Q2NM},
—C(═O)OR^{Q2NC},
L^{Q2N}-NH₂, -L^{Q2N}-NHR^{Q2NC}-L^{Q2N}-NR^{Q2NC}₂, -L^{Q2N}-R^{Q2NM},
—C(═O)NH₂, —C(═O)NHR^{Q2NC}, —C(═O)NR^{Q2NC}₂, —C(═O)R^{Q2NM},
L^{Q2N}-C(═O)NH₂, -L^{Q2N}-C(═O)NHR^{Q2NC}, -L^{Q2N}-C(═O)NR^{Q2NC}₂, -L^{Q2N}-C(═O)R^{Q2NM}, or —S(═O)₂R^{Q2NC};
wherein:
each —R^{Q2NC} is independently linear or branched saturated C₁₋₆alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₆alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
each -L^{Q2N}- is independently linear or branched saturated C₁₋₄alkylene;
each —R^{Q2NM} is independently non-aromatic C₃₋₁₁heterocyclyl having at least one N ring atom, and is attached via that N ring atom; and is:
optionally substituted on carbon with one or more groups —R^{Q2NMM};
optionally substituted on sulfur, if present, with one or two ═O groups; and
optionally substituted on secondary nitrogen, if present, with a group selected from: —R^{Q2NMM}, —C(═O)R^{Q2NMM}, —C(═O)OR^{Q2NMM}, —C(═O)NH₂, —C(═O)NHR^{Q2NMM}, —C(═O)NR^{Q2NMM}₂, and —S(═O)₂R^{Q2NMM}; and
each —R^{Q2NMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
each —R^{Q3C} is independently:
—F, —Cl, —Br, —I,
—R^{Q3CC},
—R^{Q3CX} CX—OR^{Q3CX} CX
—OH, —OR^{Q3CC},
-L^{Q3C}—OH, -L^{Q3C}-OR^{Q3CC},
—NH₂, —NHR^{Q3CC}, —NR^{Q3CX} CC₂, -RQ³ CM
-L^{Q3C}-NH₂, -L^{Q3C}-NHR^{Q3CC}, -L^{Q3C}-NR^{Q3CX} CC₂, -L^{Q3C}-Q³ CM,
—NHC(═O)R^{Q3CC}, —NHC(═O)OR^{Q3CC},
L^{Q3C}-C(═O)R^{Q3CXC}, -L^{Q3C}—NHC(═O)OR^{Q3CC},
—C(═O)NH₂, —C(═O)NHR^{Q3CC}, —C(═O)NR^{Q3CXCC}₂, —C(═O)R^{Q3CM},
-L^{Q3C}-C(═O)NH₂, -L^{Q3C}-C(═O)NHR^{Q3CC}, -L^{Q3C}—C(═O)NR^{Q3CXCC}₂, -L^{Q3C}-C(═O)RQ³ CM
—C(═O)OH, —C(═O)OR^{Q3CC},
—OC(═O)R^{Q3CC},
—OC(═O)NH₂, —OC(═O)NHR^{Q3CC}, —OC(═O)NR^{Q3CXCC}₂, —OC(═O)RQ³ CM,
—S(═O)₂R^{Q3CC}, S(═O)₂R^{Q3CM},
—CN, or —NO₂;
and two adjacent-RO3C, if present, taken together may
form —(CH₂)ₙ₃—O—(CH₂)ₘ₃—, —(CH₂)ₙ₃—NH—(CH₂)ₘ₃—, —(CH₂)_{q3} (C(O))—NH—(CH₂)ᵥ₃—, —NH—(CH₂)_{q3}C(O)(CH₂)ᵥ₃—O—, —NH—(CH₂)ₚ₃—NH—or —O—(CH₂)ₚ₃—O—, wherein: n3 is 0, 1, 2, or 3;
m3 is 0, 1, 2, or 3; p3 is 1 or 2; q3 is 0, 1, 2, or 3; v3 is 0, 1, 2, or 3 with the proviso that
m3+n3 is 2 or 3;
wherein:
each —R^{Q3CC} is independently linear or branched saturated C₁₋₄alkyl, C₃₋₆cycloalkyl, C₃₋₆Cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —F, —OH or —OCH₃, and each cycloalkyl, phenyl and heteroaryl is optionally substituted with one or more groups selected from: —F, —Cl, —CH₃, —CF₃, —OH, —OCH₃, —NH₂,
—NH(CH₃), and —N(CH₃)₂;
each —R^{Q3CX} is independently linear or branched saturated C₁₋₄haloalkyl;
each -L^{Q3C}- is independently linear or branched saturated C₁₋₄alkylene;
each -RQ³ CM is independently non-aromatic C₃₋₁₁heterocyclyl having at least one N ring atom, and is attached via that N ring atom; and is:
optionally substituted on carbon with one or more groups —R^{Q3CCMM}, optionally substituted on sulfur, if present, with one or two ═O groups; and
optionally substituted on secondary nitrogen, if present, with a group selected from: —R^{Q3CCMM}, —C(═O)R^{Q3CCMM}, —C(═O)OR^{Q3CCMM}, —C(═O)NH₂, —C(═O)NHR^{Q3CMM}, —C(═O)NR^{Q3CMM}₂, and —S(═O)₂R^{Q3CCMM}; and
each —R^{Q3CMM} is independently linear or branched saturated-F, C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl,
wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
each —R^{Q4C} is independently:
—F,
—R^{Q4CC}, —R^{Q4CX},
—OH, —OR^{Q4CC}, —OR^{Q4CX},
—NH₂, —NHR^{Q4CC}, —NR^{Q4CC}₂, -RQ⁴ CM
—NHC(═O)R^{Q4CC}, —NHC(═O)OR^{Q4CC},
—C(═O)NH₂, —C(═O)NHR^{Q4CC}, —C(═O)NR^{Q4CC}₂, —C(═O)RQ⁴ CM,
—C(═O)OH, —C(═O)OR^{Q4CC},
—OC(═O)R^{Q4CC},
—OC(═O)NH₂, —OC(═O)NHR^{Q4CC}, —OC(═O)NR^{Q4CC}₂, —OC(═O)RQ⁴ CM, or
═O;
wherein:
each —R^{Q4CC} is independently linear or branched saturated C₁₋₄alkyl, C₃₋₆cycloalkyl, C₃₋₆Cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
each —R^{Q4CX} is independently linear or branched saturated C₁₋₄haloalkyl;
each -RQ⁴ CM is independently non-aromatic C₃₋₁₁heterocyclyl having at least one N ring atom, and is attached via that N ring atom; and is:
optionally substituted on carbon with one or more groups —R^{Q4CMM};
optionally substituted on sulfur, if present, with one or two ═O groups; and
optionally substituted on secondary nitrogen, if present, with a group selected from: -RQ⁴ CM, —C(═O)R^{Q4CMM}, —C(═O)OR^{Q4CM}, —C(═O)NH₂, —C(═O)NHR^{Q4CMM}, —C(═O)NR^{Q4CMM}₂, and —S(═O)₂R^{Q4CMM};
each —R^{Q4CMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl,
wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
and wherein:
each —R^{Q5C} is independently:
—F, —OH, —OR^{Q5CC}, —OCF₃,
—NH₂, —NHR^{Q5CC}, —NR^{Q5CC}₂, -RQ⁵ CM
—NHC(═O)R^{Q5CC}, —NHC(═O)OR^{Q5CC},
—C(═O)NH₂, —C(═O)NHR^{Q5CC}, —C(═O)NR^{Q5CC}₂, —C(═O)R^{Q5CM},
—C(═O)OH, —C(═O)OR^{Q5CC},
—OC(═O)R^{Q5CC},
—OC(═O)NH₂, —OC(═O)NHR^{Q5CC}, —OC(═O)NR^{Q5CC}₂, or —OC(═O)R^{Q5CM},
wherein:
each —R^{Q5CC} is independently linear or branched saturated C₁₋₄alkyl, C₃₋₆cycloalkyl, C₃₋₆cycloalkyl-C₁₋₃alkyl, C₃₋₇heterocyclyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and
each cycloalkyl, phenyl and heteroaryl is optionally substituted with one or more groups selected from: —F, —Cl, —CH₃, —CF₃, —OH, —OCH₃, —NH₂, —NH(CH₃), and —N(CH₃)₂;
each —RO5 CM is independently non-aromatic C₃₋₁₁heterocyclyl having at least one N ring atom, and is attached via that N ring atom; and is:
optionally substituted on carbon with one or more groups —R^{Q5CMM},
optionally substituted on sulfur, if present, with one or two ═O groups; and
optionally substituted on secondary nitrogen, if present, with a group selected from: —R^{Q5CMM}, —C(═O)R^{Q5CMM}, —C(═O)OR^{Q5CMM}, —C(═O)NH₂, —C(═O)NHR^{Q5CMM}, —C(═O)NR^{Q5CMM}₂, and —S(═O)₂R^{Q5CMM};
each —R^{Q5CMM} is independently linear or branched saturated C₁₋₄alkyl, C₃₋₆cycloalkyl, C₃₋₆cycloalkyl-C₁₋₃alkyl, phenyl, phenyl-C₁₋₃alkyl, C₅₋₆heteroaryl, or C₅₋₆heteroaryl-C₁₋₃alkyl, wherein C₁₋₄alkyl is optionally substituted with —OH or —OCH₃, and each cycloalkyl, phenyl and heteroaryl is optionally substituted with one or more groups selected from: —F, —Cl, —CH₃, —CF₃, —OH, —OCH₃, —NH₂,
—NH(CH₃), and —N(CH₃)₂.

2. A compound according to claim 1 or a pharmaceutically acceptable salt or solvate thereof wherein:
—R^{A1} is —R^{A11}; and
—R^{A11} is —R^{A111}, —F, —Cl, or —Br; and
each —R^{A111} is independently linear or branched saturated C₁₋₄alkyl.

3. A compound according to any one of the preceeding claims or a pharmaceutically acceptable salt or solvate thereof wherein:
—R^{A2} is —R^{A22};
—R^{A22} is —R^{A222}, —F, —Cl, or —Br; and
each —R^{A222} is independently linear or branched saturated C₁₋₄alkyl.

4. A compound according to any one of the preceeding claims or a pharmaceutically acceptable salt or solvate thereof wherein:
—R^{A3} is —H or —R^{A33}; and
—R^{A33} is —Br.

5. A compound according to any one of the preceeding claims or a pharmaceutically acceptable salt or solvate thereof wherein:
—R^{A4} is —H or —R^{A44};
—R^{A44} is —R^{A444}, —Cl, or —Br; and
each —R^{A444} is independently linear or branched saturated C₁₋₄alkyl.

6. A compound according to any one of the preceeding claims or a pharmaceutically acceptable salt or solvate thereof wherein:
—Ring B is selected from: 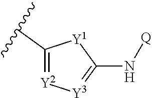
wherein:
Y¹ is S, or O;
Y² is CH, CR^{Y2}, or N;
Y³ is N; and
each —R^{Y2} is independently —NH₂.

7. A compound according to any one of the preceeding claims or a pharmaceutically acceptable salt or solvate thereof wherein:
-Q is -Q¹, or -L^{Q1}-Q¹; wherein:
Q¹ is C₅₋₁₀heteroaryl;
and is:
optionally substituted on carbon with one or more groups —R^{Q1C}; and
optionally substituted on secondary nitrogen, if present, with one or more groups —R^{Q1N}; and
-L^{Q1}- is linear or branched saturated C₁₋₄alkylene;
wherein
each —R^{Q1C} is independently:
—F, —Cl, —Br,
—R^{Q1CC},
—R^{Q1CX}, —OR^{Q1CX},
—OH, —OR^{Q1CC},
—NH₂, —NHR^{Q1CC}, —NR^{Q1CG}₂, -RQ¹ CM
L^{Q1C}-RQ¹ CM
—NHC(═O)R^{Q1CC}, —N(R^{Q1CC})C(═O)R^{Q1CC}, —NHC(═O)OR^{Q1CC},
—C(═O)NH₂, —C(═O)NHR^{Q1CC}, —C(═O)NR^{Q1CC}₂, —C(═O)R^{Q1CM}, ═O,
—NHC(═O)NHR^{Q1CC},
-L^{Q1C}—C(═O)NR^{Q1CC}₂,
—C(═O)OH, —C(═O)OR^{Q1CC},
—OC(═O)NH₂,
—S(═O)₂R^{Q1CX},
—S(═O)₂R^{Q1CM},
—NHS(═O)R^{Q1CC}, —NHS(═O)₂R^{Q1CC},
—CN, or —C≡CH;
wherein:
each —R^{Q1CC} is independently linear or branched saturated C₁₋₆alkyl, C₂₋₆alkenyl, C₃₋₆cycloalkyl, C₃₋₇heterocyclyl, phenyl, or C₅₋₆heteroaryl, wherein C₁₋₆alkyl is optionally substituted with —OH, —CN, or —OCH₃, and each phenyl and heteroaryl is optionally substituted with one or more groups selected from: —F, —Cl, —CH₃, —CHF₂, —CF₃, —OCH₃;
each —R^{Q1CX} is independently linear or branched saturated C₁₋₄haloalkyl;
each -L^{Q1C}- is independently linear or branched saturated C₁₋₄alkylene;
each —R^{Q1CM} is independently non-aromatic C₃₋₁₁heterocyclyl having at least one N ring atom, and is attached via that N ring atom; and is:
optionally substituted on carbon with one or more groups —R^{Q1CMM};
optionally substituted on secondary nitrogen, if present, with a group selected from: —R^{Q1CMM}; and
each —R^{Q1CMM} is independently —F, —NH₂, linear or branched saturated C₁₋₄alkyl, C₁₋₄alkyIOC(═O)NH—, C₃₋₆cycloalkyl, wherein C₁₋₄alkyl is optionally substituted with —OH;
and wherein:
each —R^{Q1N} is independently:
—R^{Q1NC} -L^{Q1N}-R^{Q1NC},
—R^{Q1NX},
—R^{Q1Nhet}-L^{Q1N}-R^{Q1Nhet},
-L^{Q1N}-OH, -L^{Q1N}-OR^{Q1NC},
—S(═O)₂R^{Q1NC},
-L^{Q1N}-C(═O)OH, -L^{Q1N}-C(═O)OR^{Q1NC},
-L^{Q1N}-C(═O)NH₂, -L^{Q1N}-C(═O)NHR^{Q1NK}, -L^{Q1N}-C(═O)NR^{Q1NC}₂, -L^{Q1N}-C(═O)R^{Q1NP},
-L^{Q1N}-NH₂, -L^{Q1N}-NR^{Q1NC};
or -L^{Q1N}—NHC(═O)OR^{Q1NC};
wherein:
each —R^{Q1NC} is independently linear or branched saturated C₁₋₄alkyl, C₃₋₆cycloalkyl, phenyl, or C₅₋₆heteroaryl, wherein each C₁₋₄alkyl is optionally substituted by —F, —OH, —C≡N, —SO₂—CH₃, or —OCH₃, wherein each cycloalkyl, phenyl and heteroaryl is optionally substituted with one or more groups selected from: —F, —Cl, —Br, linear or branched saturated C₁₋₄alkyl, —CHF₂, —CF₃, —CH₂—O—CH₃, —OCH₂CH₃, —C(═O)—NH—phenyl, wherein C₁₋₄alkyl and phenyl are optionally substituted by —CH₃ or —OH;
each —R^{Q1NX} is independently linear or branched saturated C₁₋₄haloalkyl;
each -L^{Q1N}- is independently linear or branched saturated C₁₋₄alkylene wherein C₁₋₄alkylene is optionally substituted by —OH or —OMe;
and wherein:
each —R^{Q1Nhet} is independently non-aromatic C₃₋₇heterocyclyl; and is:
optionally substituted on carbon with one or more or ═O; and
optionally substituted on secondary nitrogen, if present, with a group selected from: —R^{Q1NHH}, —C(═O)R^{Q1NJJ}, —C(═O)OR^{Q1NHH}, —C(═O)NHR^{Q1NHH}, —C(═O)NR^{Q1NHH}₂;
wherein:
each —R^{Q1NHH} is independently linear or branched saturated C₁₋₄alkyl, C₃₋₆cycloalkyl, phenyl-C₁₋₃alkyl;
and wherein:
—R^{Q1NJJ} is —R^{J1}, -L^{J}-R^{J2}, —R^{J3}, -L^{J}-R^{J3}, —R^{J4}, -L^{J}-R^{J4}, or -L^{J}-R^{J5};
—R^{J1} is linear or branched saturated C₁₋₆alkyl; and is optionally substituted with one or more groups selected from: —F, —OH, —OR″, —O-phenyl, —C(═O)OR^{JJ};
each —R^{J2} is independently C₃₋₆cycloalkyl;
each —R^{J3} is independently non-aromatic C₃₋₇heterocyclyl;
each —R^{J4} is phenyl; and is optionally substituted with one or more groups selected from: —F, —OR^{J}, —NH₂, and —NR^{J2};
each —R^{J5} is independently C₅₋₆heteroaryl;
each -L^{J}- is independently linear or branched saturated C₁₋₄alkylene, and is optionally substituted with one or more-F;
each —R^{J} is linear or branched saturated C₁₋₄alkyl;
and wherein:
—R^{Q1NK} is —R^{K1}, —R^{K2}, —R^{K3}, -L^{K}-R^{K3};
—R^{K1} is linear or branched saturated C₁₋₇alkyl; and is optionally substituted with one or more groups selected from: —F, —OH, —OR^{KK}, —OCH₂CH₂OCH₃, and —NR^{KK}₂;
each —R^{K2} is independently C₃₋₆cycloalkyl;
each —R^{K3} is independently non-aromatic C₃₋₇heterocyclyl; and is:
optionally substituted on secondary nitrogen, if present, with a group selected from: —R^{KK}, and —C(═O)OR^{KK};
each -L^{K}- is linear or branched saturated C₁₋₄alkylene,
each —R^{KK} is linear or branched saturated C₁₋₄alkyl;
and wherein:
—R^{Q1NP} is non-aromatic C₃₋₁₁heterocyclyl having at least one N ring atom, and is attached via that N ring atom; and is:
optionally substituted on carbon with one or more groups selected from —R^{Q1NPP}, —F, —OH, —OR^{Q1NPP}, and ═O; and
optionally substituted on secondary nitrogen, if present, with a group selected from: —R^{Q1NPP}, —R^{Q1NPPX}, —C(═O)R^{Q1NPP}, —C(═O)OR^{Q1NPP}, —C(═O)NR^{Q1NPP}₂, and —S(═O)₂R^{Q1NPP};
each —R^{Q1NPP} is independently linear or branched saturated C₁₋₄alkyl, C₃₋₆cycloalkyl, C₃₋₆cycloalkyl-C₁₋₃alkyl, phenyl, wherein C₁₋₄alkyl is optionally substituted with —F, —OH or —OCH₃;
—R^{Q1NPPX} is linear or branched saturated C₁₋₄haloalkyl.

8. A compound according to any one of the claims 1-6 or a pharmaceutically acceptable salt or solvate thereof wherein:
Q is -Q², or -L^{Q2}-Q²; wherein
Q² is C₃₋₁₀heterocyclyl;
and is:
optionally substituted on sulfur, if present, with one or two groups ═O;
optionally substituted on secondary nitrogen, if present, with one or more groups —R^{Q2N}; and
-L^{Q2}- is linear or branched saturated C₁₋₄alkylene;
wherein
each —R^{Q2N} is independently:
—R^{Q2NC},
═O, —C(═O)R^{Q2NC},
—C(═O)-L^{Q2N}-R^{Q2NM},
—C(═O)OR^{Q2NC},
-L^{Q2N}-C(═O)NR^{Q2NC}₂, or
—S(═O)₂R^{Q2NC};
wherein:
each —R^{Q2NC} is independently linear or branched saturated C₁₋₆alkyl, phenyl-C₁₋₃alkyl, or C₅₋₆heteroaryl-C₁₋₃alkyl, wherein C₁₋₆alkyl is optionally substituted with —OH, and each, phenyl and heteroaryl is optionally substituted with one or more groups selected from: —Cl, and —OCH₃;
each -L^{Q2N} is independently linear or branched saturated C₁₋₄alkylene;
each —R^{Q2NM} is independently non-aromatic C₃₋₁₁heterocyclyl having at least one N ring atom, and is attached via that N ring atom.

9. A compound according to any one of the claims 1-6 or a pharmaceutically acceptable salt or solvate thereof wherein:
-Q is -Q³, or -L^{Q3}-Q³;
Q³ is phenyl;
and is optionally substituted with one or more groups —R^{Q3C}; and
-L^{Q3}- is linear or branched saturated C₁₋₄alkylene;
and wherein:
each —R^{Q3C} is independently:
—F,
—R^{Q3CX} CC
—R^{Q3CX} CX,
—OH, —OR^{Q3CC},
—NHC(═O)R^{Q3CX} CC
—C(═O)NHR^{Q3CC}, —C(═O)RQ³ CM,
—S(═O)₂R^{Q3CC}, S(═O)₂RQ³ CM;
and two adjacent-R93C, if present, taken together may form —NH—(CH₂)_{q3}C(O)(CH₂)ᵥ₃—O—, wherein q3 is 0 and v3 is 1;
wherein:
each —R^{Q3CC} is independently linear or branched saturated C₁₋₄alkyl, C₃₋₆cycloalkyl, C₃₋₇heterocyclyl, wherein C₁₋₄alkyl is optionally substituted with —F;
each —R^{Q3CX} is independently linear or branched saturated C₁₋₄haloalkyl;
each -RQ³ CM is independently non-aromatic C₃₋₁₁heterocyclyl having at least one N ring atom, and is attached via that N ring atom; and is:
optionally substituted on carbon with one or more groups —R^{Q3CCMM};
each —R^{Q3CCMM} is independently —F.

10. A compound according to any one of the claims 1-6 or a pharmaceutically acceptable salt or solvate thereof wherein:
Q is -Q⁴, or -L^{Q4}-Q⁴,
Q⁴ is C₃₋₁₀carbocyclyl; and
-L^{Q4}- is linear or branched saturated C₁₋₄alkylene.

11. A compound according to any one of the claims 1-6 or a pharmaceutically acceptable salt or solvate thereof wherein:
-Q is -Q⁵ or —H;
Q⁵ is linear or branched saturated C₁₋₆alkyl;
and is optionally substituted with one or more groups —R^{Q5C};
wherein
each —R^{Q5CC} is independently:
—OH, —OR^{Q5CC},
—NHC(═O)R^{Q5CC}, —NHC(═O)OR^{Q5CC},
—C(═O)NH₂, —C(═O)NHR^{Q5CC}, —C(═O)R^{Q5CM},
wherein:
each —R^{Q5CC} is independently linear or branched saturated C₁₋₄alkyl;
each -RQ⁵ CM is independently non-aromatic C₃₋₁₁heterocyclyl having at least one N ring atom, and is attached via that N ring atom.

12. A pharmaceutical composition comprising:
a compound according to any one of claims 1-11, or
a pharmaceutically acceptable salt or solvate thereof, and
a pharmaceutically acceptable carrier or diluent.

13. A compound according to any one of claims 1-11, or a pharmaceutically acceptable salt or solvate thereof, for use in a method of treatment of the human or animal body by therapy.

14. Use of a compound according to any one of claims 1-11, or a pharmaceutically acceptable salt or solvate thereof, in the manufacture of a medicament for the treatment of a proliferative disorder.

15. A method of treatment of a proliferative disorder of the human or animal body, comprising administering to a subject in need of treatment a therapeutically-effective amount of a compound according to any one of claims 1-11, or a pharmaceutically acceptable salt or solvate thereof.

16. A compound, salt, or solvate for use according to claim 14, or
a method according to claim 15,
wherein the proliferative disorder is cancer.

17. A compound, salt, or solvate for use according to claim 14, or
a method according to claim 15,
wherein the proliferative disorder is: endometrial cancer, uterine cancer, ovarian cancer, breast cancer, gastric cancer, bladder cancer, pancreatic cancer, mesothelioma, kidney cancer, stomach cancer, esophageal cancer, colorectal cancer, glioblastoma, or lung cancer.
